(19) United States
(12) Patent Application Publication
(10) Pub. No.: US 2024/0317721 A1
(43) Pub. Date: Sep. 26, 2024

(54) PYRAZOLYL DERIVATIVES AS INHIBITORS OF THE KRAS MUTANT PROTEIN

(71) Applicant: NOVARTIS AG, Basel (CH)

(72) Inventors: Claudio BOMIO-CONFAGLIA, Basel (CH); Saskia Maria BRACHMANN, Basel (CH); Simona COTESTA, Basel (CH); Marc GERSPACHER, Basel (CH); Catherine LEBLANC, Basel (CH); Fabio LIMA, Basel (CH); Edwige Liliane Jeanne LORTHIOIS, Basel (CH); Rainer MACHAUER, Basel (CH); Robert MAH, Basel (CH); Sophie RACINE, Basel (CH); Pascal RIGOLLIER, Basel (CH); Stefan STUTZ, Basel (CH); Andrea VAUPEL, Basel (CH); Nicolas WARIN, Basel (CH); Rainer WILCKEN, Basel (CH); Frédéric ZECRI, Cambridge, MA (US)

(21) Appl. No.: 18/571,588

(22) PCT Filed: Jun. 22, 2022
(86) PCT No.: PCT/IB2022/055789

§ 371 Date: Dec. 18, 2023

Related U.S. Application Data
(60) Provisional application No. 63/214,009, filed on Jun. 23, 2021.

(51) Int. Cl. C07D 403/14; A61K 31/416; A61K 31/4162; A61K 31/437; A61K 31/438; A61K 31/4439; A61K 31/454; A61K 31/496; A61K 31/4985; A61K 31/499; A61K 31/4995; A61K 31/501; A61K 31/5025; A61K 31/519; A61K 31/5377; A61K 31/5383; A61K 31/5386; A61K 31/541; A61K 31/547; A61K 31/55; A61K 31/551; A61K 31/553; A61K 31/675; C07D 401/14; C07D 405/14; C07D 413/14; C07D 417/14; C07D 471/04; C07D 471/10; C07D 487/04; C07D 487/08; C07D 491/048; C07D 491/107; C07D 495/10; C07D 498/04; C07D 498/08; C07D 498/10; C07D 513/08; C07F 9/6584

(52) U.S. Cl. CPC C07D 403/14; A61K 31/416; A61K 31/4162; A61K 31/437; A61K 31/438; A61K 31/4439; A61K 31/454; A61K 31/496; A61K 31/4985; A61K 31/499; A61K 31/4995; A61K 31/501; A61K 31/5025; A61K 31/519; A61K 31/5377; A61K 31/5383; A61K 31/5386; A61K 31/541; A61K 31/547; A61K 31/55; A61K 31/551; A61K 31/553; A61K 31/675; C07D 401/14; C07D 405/14; C07D 413/14; C07D 417/14; C07D 471/04; C07D 471/10; C07D 487/04; C07D 487/08; C07D 491/048; C07D 491/107; C07D 495/10; C07D 498/04; C07D 498/08; C07D 498/10; C07D 513/08; C07F 9/6584

(57) ABSTRACT

The present invention provides a compound of formula (I), or a pharmaceutically acceptable salt thereof, and the therapeutic uses of said compound. The present invention further provides a pharmaceutical composition comprising said compound.

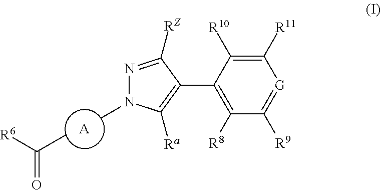

### SEQUENCE LISTING

The instant application contains a Sequence Listing which has been submitted electronically in ASCII format. Said ASCII copy, created on Jun. 3, 2022, is named PAT059119-WO-PCT SQL_ST25, is 7,086 bytes in size is filed herewith and is incorporated herein by reference.

### FIELD OF THE INVENTION

The invention provides pyrazolyl derivative compounds, compositions including said compounds, the use thereof for inhibiting KRAS G12C, HRAS G12C or NRAS G12C, methods of treating or preventing disease, in particular, cancer, using said compounds. The invention also provides such pyrazolyl derivative compounds for use in the treatment of cancer and specific cancers as defined herein.

### BACKGROUND OF THE INVENTION

RAS are small GTPases acting as molecular ON/OFF switches, which adopt an active/inactive state when bound to GTP/GDP, respectively. In response to growth factors, guanine exchange factors exchange GDP for GTP, turning Ras ON. RAS bound to GTP adopts conformations that recruit effector proteins to the plasma membrane, thereby activating signaling cascades causing cell growth, proliferation and survival. These cancer promoting signals are very transient and tightly controlled. They are turned off immediately by the GTPase activity of RAS itself, mainly due to the 100000 fold acceleration by GTPase activating proteins (GAPs) (Bos J L et al., Cell, Volume 129, Issue 5, 1 Jun. 2007, pp 865-877). In contrast, RAS mutants are insensitive to these GAPs, causing the RAS mutants to reside longer in the GTP bound state and shifting the GTP/GDP cycle in accordance to their intrinsic hydrolysis rate towards the ON state.

The three RAS genes constitute the most frequently mutated gene family in cancer, with RAS mutations found in ˜25% of human tumors. Among the 3 paralogs, KRAS mutations are most frequent (85% of all RAS-driven cancers), whereas NRAS and HRAS mutations are less frequently reported (12% and 3%, respectively). The majority of KRAS mutations occurs at the hotspot residues G12, G13 and Q61. KRAS G12C mutations represent about 12% of all KRAS mutations and are prevalent in lung cancer patients (˜13% lung adenoma carcinoma (LUAC)), ˜3-5% colon adenocarcinomas, a smaller fractions of other cancer types and in about 20% of MYH polyposis colorectal adenomas (COSMIC v80 database; A. Aime' et al, Cancer genet. 2015, 208:390-5).

Patients with KRAS G12C positive solid tumors are only poorly treated with current therapies. There are currently no inhibitors of KRAS G12C, HRAS G12C or NRAS G12C approved for therapeutic use.

There thus remains a continued need to develop new options for the treatment of cancer, in particular, cancer tumors expressing G12C mutant Ras, in particular, for the treatment of KRAS, HRAS or NRAS G12C driven cancers.

Irreversible RAS G12C inhibitors have been previously described (for example WO2014152588, WO2017201161, WO2018119183).

### SUMMARY OF THE INVENTION

The compounds described in this invention selectively react with, and inhibit, the G12C mutant KRAS, HRAS or NRAS proteins by forming an irreversible covalent bond with the cysteine at the position 12. This locks the RAS mutant protein in the inactive state. The irreversible binding of these compounds disrupts K-RAS downstream signaling. The compounds described in this invention maybe be used for the treatment of cancer, particularly the treatment of a cancer characterized by a KRAS, HRAS or NRAS G12C mutation.

The invention therefore provides compounds, pharmaceutically acceptable salts thereof, pharmaceutical compositions thereof and combinations thereof, and may be useful or the treatment of cancer, particularly the treatment of cancer characterized by a KRAS HRAS or NRAS G12C mutation.

According to a first aspect of the invention, there is hereby provided a compound of formula (I) or a pharmaceutically acceptable salt thereof:

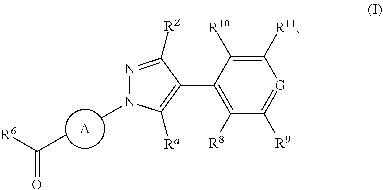

wherein:
Ring A is a 6 to 10 membered spirocyclic-heterocyclylene comprising 1 to 3 heteroatoms independently selected from N, O and S, wherein said 6 to 10 membered spirocyclic-heterocyclylene is substituted with 0 to 3 substituents R¹⁶;
G is N or CR¹²;
R^{Z} is

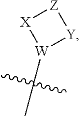

wherein
W is N;
i) X is **—CR²₂—(CR³₂)ₙ—* or **—CR²═CR³—*, Y is **—CR⁴₂—(CR⁵₂)ₘ—*, and Z is selected from the group consisting of S(O)₂, S, S(O), O, P(O)—C₁-C₃alkyl, NR^{1N} and C(R^{1C})₂, where the * of X indicates the point of attachment to Z and the ** of X indicates the point of attachment to W, and where the * of Y indicates point of attachment to Z and the ** of Y indicates point of attachment to W, n is 0, 1 or 2 and m is 0, 1 or 2; or
ii) X is **—CR²²—CR³═*, Y is **—CR⁴₂—(CR⁵₂)ₘ—*, and Z is selected from the group consisting of N and CR^{1C}, where the * of X indicates the point of attachment to Z and the ** of X indicates the point of attachment to W, and where the * of Y indicates point of attachment to Z and the ** of Y indicates point of attachment to W and m is 0, 1 or 2;
R^{1N} is selected from the group consisting of H and -L^{N}-R^{2N}, preferably wherein R^{1N} is -L^{N}-R^{2N} or
an R^{1N} group and one or two R³ groups, in combination with the atoms to which they are mutually attached, form a saturated or unsaturated 5 or 6 membered ring containing one to three heteroatoms (for example one or two heteroatoms) selected from the group consisting of N, O, S and P (preferably selected from the group consisting of N, O and S) (e.g. wherein the 5 or 6 membered ring is a 5-6 membered heterocyclyl or a 5-6 membered heteroaryl), wherein said saturated or unsaturated 5 or 6 membered ring containing one to three heteroatoms is substituted with 0 to 3 substituents R^{x}; or
an R^{1N} group and one or two R⁵ groups, in combination with the atoms to which they are mutually attached, form a saturated or unsaturated 5 or 6 membered ring containing one to three heteroatoms (for example one or two heteroatoms) selected from the group consisting of N, O, S and P (preferably selected from the group consisting of N, O and S) (e.g. wherein the 5 or 6 membered ring is a 5-6 membered heterocyclyl or a 5-6 membered heteroaryl), wherein said saturated or unsaturated 5 or 6 membered ring containing one to three heteroatoms is substituted with 0 to 3 substituents R^{x};
R^{1C}, where present, is at each occurrence independently selected from the group consisting of H and -L^{C}-R^{2C}; and/or
one or two R^{1C} group(s), and one or two R³ groups, in combination with the carbon atoms to which they are mutually attached, form a saturated or unsaturated 5 or 6 membered ring containing zero to three heteroatoms (for example one to three heteroatoms, for example one or two heteroatoms) selected from the group consisting of N, O, S and P (preferably selected from the group consisting of N, O and S) (e.g. wherein the 5 or 6 membered ring is a C₅-C₆cycloalkyl, 6 membered aryl, a 5-6 membered heterocyclyl or a 5-6 membered heteroaryl), wherein said saturated or unsaturated 5 or 6 membered ring containing zero to three heteroatoms is substituted with 0 to 3 substituents R^{x}; or
one or two R^{1C} groups, and one or two R⁵ groups, in combination with the carbon atoms to which they are mutually attached, form a saturated or unsaturated 5 or 6 membered ring containing zero to three heteroatoms (for example one to three heteroatoms, for example one or two heteroatoms) selected from the group consisting of N, O, S and P (preferably selected from the group consisting of N, O and S) (e.g. wherein the 5 or 6 membered ring is a C₅-C₆cycloalkyl, 6 membered aryl, a 5-6 membered heterocyclyl or a 5-6 membered heteroaryl), wherein said saturated or unsaturated 5 or 6 membered ring containing zero to three heteroatoms is substituted with 0 to 3 substituents R^{x}; or
two R^{1C} groups together form oxo; or
two R^{1C} groups together with the carbon atom to which they are mutually attached form a C₄-C₆cycloalkyl or a 4 to 6 membered heterocyclyl comprising 1 to 3 heteroatoms independently selected from N, O, S and P (preferably selected from the group consisting of N, O and S), said C₄-C₆cycloalkyl or 4 to 6 membered heterocyclyl being substituted with 0 to 2 substituents R^{x};
L^{N} is selected from the group consisting of a bond, C═O, C₁-C₆alkylene, SO₂, C(═O)—O*, C(═O)—C₁-C₆alkylene*, C₁-C₆alkylene-C(═O)* and C(═O)—O—C₁-C₆alkylene*, wherein * indicates the point of attachment to R^{2N}, (e.g. L^{N} is a bond);
R^{2N} is selected from the group consisting of:
   i) C₁-C₆alkyl substituted with 0 to 3 substituents R^{x} (e.g. unsubstituted C₁-C₆alkyl),
   ii) 3-10 membered heterocyclyl comprising 1 to 3 heteroatoms independently selected from N, O, S and P (preferably selected from the group consisting of N, O and S) substituted with 0 to 3 (e.g. 0 to 2) substituents R^{x},
   iii) 6 to 10 membered spirocyclic-heterocyclyl comprising 1 to 3 heteroatoms independently selected from N, O, S and P (preferably selected from the group consisting of N, O and S) substituted with 0 to 3 substituents R^{x},
   iv) hydroxyl,
   v) C₁-C₆haloalkyl,
   vi) aryl substituted with 0 to 2 substituents R^{x},
   vii) O—C₁-C₆haloalkyl,
   viii) O—C₁-C₆alkyl,
   ix) 5-6 membered heteroaryl comprising 1 to 3 (e.g. 1 or 2) heteroatoms independently selected from N, O and S substituted with 0 to 2 substituents R^{x},
   x) C₃-C₈cycloalkyl substituted with 0 to 2 substituents R^{x},
   xi) N(C₁-C₆alkyl)₂ or NH(C₁-C₆alkyl) (preferably N(C₁-C₆alkyl)₂),
   xii) CH(C₁-C₆alkylene-O—C₁-C₆alkyl)₂, and
   xiii) CN;
L^{C} is selected from the group consisting of a bond, C═O, C₁-C₆alkylene or O—C₁-C₆alkylene*, wherein * indicates the point of attachment to R^{2C}, (e.g. L^{C} is C₁alkylene);
wherein R^{2C} is at each occurrence independently selected from the group consisting of
   i) C₁-C₆ alkyl substituted by 0 to 3 substitutents R^{x} (e.g. unsubstituted C₁-C₆ alkyl),
   ii) hydroxyl,
   iii) 6 to 10 membered spirocyclic-heterocyclyl comprising 1 to 3 heteroatoms independently selected from N, O, S and P (preferably selected from the group consisting of N, O and S) substituted by 0 to 3 substitutents R^{x},
   iv) 5-6 membered heteroaryl comprising 1 to 3 (e.g. 1 or 2) heteroatoms independently selected from N, O and S substituted by 0 to 2 substitutents R^{x},
   v) 3-10 membered heterocyclyl comprising 1 to 3 heteroatoms independently selected from N, O, S and P, substituted by 0 to 3 (e.g. 0 to 2) substituents R^{x} or wherein the 3-10 membered heterocyclyl is perdeuterated,
   vi) NR^{1A}R^{1B}, and
   vii)

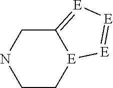

wherein E at each occasion is independently selected from CH and N substituted by 0 to 2 (e.g. 0) substitutents R^{x},
   R^{1A} and R^{1B} are each independently selected from the group consisting of H, C₁-C₆alkyl, C₁-C₆hydroxyalkyl, C₁-C₆haloalkyl, C₁-C₆alkylene-O—C₁-C₆alkyl, C₃-C₈cycloalkyl substituted by 0 to 2 substituents R^{x}, 3-10 membered heterocyclyl comprising 1 to 3 heteroatoms independently selected from N, O, S and P (preferably selected from the group consisting of N, O and S) substituted by 0 to 2 substituents R^{x}, C₁-C₆alkylene-C₃-C₈cycloalkyl substituted by 0 to 2 substituents R^{x}, C₁-C₆alkylene-3-10 membered heterocyclyl comprising 1 to 3 heteroatoms independently selected from N, O, S and P (preferably selected from the group consisting of N, O and S) substituted by 0 to 2 substituents R^{x}, SO₂-3-10 membered heterocyclyl comprising 1 to 3 heteroatoms independently selected from N, O, S and P (preferably selected from the group consisting of N, O and S) substituted by 0 to 2 substituents R^{x}, 6 to 10 membered spirocyclic-heterocyclyl comprising 1 to 3 heteroatoms independently selected from N, O, S and P (preferably selected from the group consisting of N, O and S) substituted by 0 to 2 substituents R^{x}, aryl substituted by 0 to 2 substituents R^{x}, 5-6 membered heteroaryl comprising 1 or 2 heteroatoms independently selected from N, O and S substituted by 0 to 2 substituents R^{x}, C₁-C₆alkylene-aryl substituted by 0 to 2 substituents R^{x}, C₁-C₆alkylene-5-6 membered heteroaryl comprising 1 or 2 heteroatoms independently selected from N, O and S substituted by 0 to 2 substituents R^{x}, C(═O)—C₁-C₆alkyl, C(═O)—C₁-C₆alkylene-O—C₁-C₆alkyl, and C₁-C₆alkylene-C(═O)-3-10 membered heterocyclyl comprising 1 to 3 heteroatoms independently selected from N, O, S and P (preferably selected from the group consisting of N, O and S) substituted by 0 to 2 substituents R^{x};
   R², R³, R⁴ and R⁵ are each independently selected from the group consisting of H, C₁-C₆alkyl (e.g., Me, Et, iPr), C₃-C₈cycloalkyl (preferably C₃-C₆cycloalkyl, more preferably C₃-C₅cycloalkyl, even more preferably C₃-C₄cycloalkyl), halo, C₁-C₆alkylene-O—C₁-C₆alkyl, C(═O)—C₁-C₅alkyl, C₁-C₆haloalkyl (e.g. C₁-C₆fluoroalkyl, e.g. CHF₂), hydroxyl, C₁-C₆hydroxyalkyl, NR^{1P}R^{1Q}, C₁-C₆alkylene-NR^{1P}R^{1Q}, cyano, C₁-C₆cyanoalkyl, C₁-C₆alkylene-O—C₁-C₆haloalkyl, C(═O)—NHC₁-C₅alkyl, C(═O)—N(C₁-C₅alkyl)₂, and C(═O)—O—C₁-C₅alkyl,
wherein R^{1P} and R^{1Q} are each independently selected from the group consisting of H, C(═O)—C₁-C₆alkyl, C₁-C₆alkyl, C₁-C₆alkylene-O—C₁-C₆alkyl and C₁-C₆hydroxyalkyl or wherein R^{1P} and R^{1Q} together with the nitrogen atom to which they are mutually attached form a 4 to 6 membered heterocyclyl comprising 1 or 2 heteroatoms independently selected from N, O, S;
preferably R², R³, R⁴ and R⁵ are each independently selected from the group consisting of H, C₁-C₆alkyl (e.g., Me, Et, iPr), C₃-C₈cycloalkyl (preferably C₃-C₆cycloalkyl, more preferably C₃-C₅cycloalkyl, even more preferably C₃-C₄cycloalkyl), C₁-C₆alkylene-O—C₁-C₆alkyl, C(═O)—C₁-C₅alkyl, C₁-C₆haloalkyl (e.g. C₁-C₆fluoroalkyl, e.g. CHF₂), C₁-C₆hydroxyalkyl, cyano, and C₁-C₆cyanoalkyl;
   and/or
i) an R² group and an R⁴ group in combination form a bridging group;
ii) an R² group and an R⁵ group in combination form a bridging group;
iii) an R³ group and an R⁴ group in combination form a bridging group; or
iv) an R³ group and an R⁵ group in combination form a bridging group;
wherein the bridging group forms a C₄-C₆cycloalkyl, or a 4 to 6 membered heterocyclyl comprising 1 to 3 heteroatoms independently selected from the group consisting of N, O, S and P (preferably selected from the group consisting of N, O and S), wherein the C₄-C₆cycloalkyl or 4 to 6 membered heterocyclyl are each substituted with 0 to 3 substituents R^{x}; or
   i) an R² group and an R³ group in combination with the carbon atoms to which they are mutually attached form a ring; and/or
   ii) an R⁴ group and an R⁵ group in combination with the carbon atoms to which they are mutually attached form a ring;
   wherein the ring is a C₄-C₆cycloalkyl, or a 4 to 6 membered heterocyclyl comprising 1 to 3 heteroatom independently selected from the group consisting of N, O, S and P (preferably selected from the group consisting of N, O and S), wherein the C₄-C₆cycloalkyl or 4 to 6 membered heterocyclyl are each substituted with 0 to 3 substituents R^{x}; and/or
   i) two R² groups in combination form an oxo or in combination with the carbon atom to which they are mutually attached form a ring;
   ii) two R³ groups in combination form an oxo or in combination with the carbon atom to which they are mutually attached form a ring;
   iii) two R⁴ groups in combination form an oxo or in combination with the carbon atom to which they are mutually attached form a ring;
      or
   iv) two R⁵ groups in combination form an oxo or in combination with the carbon atom to which they are mutually attached form a ring;
wherein the ring is a C₃-C₆cycloalkyl or a 3 to 6 membered heterocyclyl comprising 1 or 2 heteroatoms independently selected from the group consisting of N, O, S and P (preferably selected from the group consisting of N, O and S), wherein the C₃-C₆cycloalkyl or 3 to 6 membered heterocyclyl is substituted with 0 to 3 substituents R^{x};
   each R^{x} is independently selected from a) C₁-C₃alkyl (e.g., Me, Et, iPr), b) halo (preferably fluoro), c) C(═O)—C₁-C₃alkyl, d) C(═O)—C₁-C₃hydroxyalkyl, e) cyano, f) hydroxyl, g) amino, h) oxo, i) O—C₁-C₃alkyl, j) C₁-C₃hydroxyalkyl, k) C₁-C₃haloalkyl, l) O—C₁-C₃haloalkyl, m) COOH, n) SO₂—C₁-C₃alkyl, o) C₁-C₃alkylene-O—C₁-C₃alkyl, p) C₃-C₆cycloalkyl substituted by 0 to 2 (preferably 0) substituents selected from the group consisting of CH₃, OH, OMe, F and CN, q) 3 to 6 membered heterocyclyl comprising 1 to 3 heteroatoms (preferably 1 or 2 heteroatoms) independently selected from the group consisting of N, O and S (preferably selected from the group consisting of N and O) substituted by 0 to 2 (preferably 0) substituents selected from the group consisting of CH₃, OH, OMe, F and CN, r) NR^{Xa}R^{Xb}, s) C(═O)—NR^{Xa}R^{Xb}, and t) deuterium;
   wherein R^{Xa} and R^{Xb} are independently selected from the group consisting of H, C(═O)—C₁-C₆alkyl, SO₂—C₁-C₃alkyl, C₂-C₄haloalkyl, C₂-C₄alkylene-O—C₁-C₃alkyl, C₁-C₃alkyl and 3 to 6 membered heterocyclyl comprising 1 to 3 heteroatoms (preferably 1 or 2 heteroatoms) independently selected from the group consisting of N, O and S (preferably selected from the group consisting of N and O) (preferably wherein R^{Xa} and R^{Xb} are independently selected from the group consisting of H and C₁-C₃alkyl);
   R⁶ is CR^{7a}═CR^{7b}₂, C≡CR^{7b}, or CR^{7c}₃;
   R^{7a}, where present, is H or fluoro (preferably R^{7a} where present is H);
   each R^{7b} is independently selected from the group consisting of H, halo (preferably fluoro or chloro) and C(R^{7d})₃ wherein each R^{7d} is independently selected from the group consisting of H, halo (preferably fluoro or chloro), O—C₁-C₆alkyl, C₁-C₆alkyl, hydroxyl, and NR^{7e}R^{7f}, wherein R^{7e} and R^{7f} are each H or C₁-C₆alkyl, or wherein R^{7e} and R^{7f} together with the nitrogen atom to which they are mutually attached form a 3 to 8 membered heterocyclyl comprising 1 to 3 heteroatoms each independently selected from the group consisting of N, O, S and P, with at least one heteroatom being nitrogen, (preferably selected from the group consisting of N, O and S with at least one heteroatom being nitrogen), with the proviso that if one R^{7d} substituent is selected from the group consisting of O—C₁-C₆ alkyl, hydroxyl or NR^{7e}R^{7f} the other two R^{7d} substituents are both H;
   one R⁷⁰ is selected from the group consisting of H, halo (preferably fluoro or chloro) and C₁-C₆alkyl (preferably wherein one R^{7C} is H) and the other two R^{7c} groups in combination with the carbon atom to which they are mutually attached form a 3 membered heterocyclyl comprising 1 heteroatom selected from the group consisting of N and O (preferably wherein the heteroatom is O);
   R⁸ is H, halo (preferably chloro), O—C₁-C₃alkyl (preferably OMe), C₃-C₄cycloalkyl,

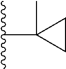 [SMILES: *C1(C)CC1]

or C(R^{8a})₃, wherein each R^{8a} is independently selected from the group consisting of H, C₁-C₃alkyl, and halo (preferably fluoro), preferably wherein each R^{8a} is H (e.g. wherein each R^{8a} is D), and
R⁹ is H, halo (preferably fluoro or chloro), NH₂, hydroxyl, C₃-C₄cycloalkyl or C(R^{9a})₃, wherein each R^{9a} is independently selected from the group consisting of H, C₁-C₃alkyl, and halo (preferably fluoro), preferably wherein each R^{9a} is H (e.g. wherein each R^{9a} is D),
or R⁸ and R⁹ together with the aryl ring to which they are mutually attached form

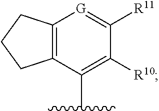

R¹⁰ is selected from the group consisting of H, halo, NH₂, C₁-C₃alkyl (preferably Me), and hydroxyl and R¹¹ is selected from the group consisting of H, halo, NH₂, hydroxyl and C₁-C₃alkyl (preferably wherein R¹¹ is H or hydroxyl); or
R¹⁰ and R¹¹ are joined together to form, in combination with the 6 membered aryl or heteroaryl to which they are mutually attached, a 9 or 10 (preferably 9) membered fused bicyclic aryl or heteroaryl group containing 1 to 3 (preferably 2) heteroatoms independently selected from the group consisting of N, O, and S (preferably wherein the heteroatom(s) are independently selected from the group consisting of N and O, more preferably wherein each heteroatom is N), wherein said fused bicyclic heteroaryl group is substituted with 0 to 3 substituents independently selected from the group consisting of C₁-C₆alkyl (preferably methyl), NH₂, R¹⁴, R¹⁵, R¹⁷, R¹⁸, R¹⁹ and R²⁰;
R¹² is H, halo (preferably fluoro) or methyl, preferably R¹² is H;
R^{a} is H, CN or C(R¹³)₃,
each R¹³ is independently selected from the group consisting of H, deuterium, halo (preferably fluoro), C₁-C₃alkyl and hydroxyl, provided that no more than one R¹³ is hydroxyl, or two R¹³ substituents in combination with the carbon atom to which they are mutually attached form a C₃-C₅cycloalkyl or a 3 to 5 membered heterocyclyl comprising 1 to 3 (preferably 1) heteroatoms each independently selected from the group consisting of N, O, S and P (preferably selected from the group consisting of N, O and S), preferably wherein the two R¹³ substituents in combination with the carbon atom to which they are mutually attached form a C₃cycloalkyl, and the third R₁₃ substituent is H, halo (preferably fluoro), C₁-C₃alkyl or hydroxyl (preferably wherein the third R₁₃ substituent is H),
R¹⁴ is selected from the group consisting of H and C₁-C₃alkyl;
R¹⁵, R¹⁷, R¹⁸, R¹⁹ and R²⁰ are each independently selected from the group consisting of H, halo, C₁-C₃alkyl and NH₂; and
each R¹⁶ group is independently selected from the group consisting of C₁-C₃alkyl, cyano, halo (e.g. fluoro), hydroxyl, O—C₁-C₃alkyl, C₁-C₃haloalkyl (e.g. C₁-C₃fluoroalkyl), C₁-C₃hydroxyalkyl, and C₁-C₃cyanoalkyl.

According to a second aspect of the invention there is hereby provided a pharmaceutical composition comprising a compound or pharmaceutically acceptable salt thereof according to the first aspect of the invention and at least one pharmaceutically acceptable carrier.

According to a third aspect of the invention there is hereby provided a compound or pharmaceutically acceptable salt thereof according to the first aspect of the invention, or a pharmaceutical composition according to the second aspect of the invention for use as a medicament (e.g. in the treatment of cancer).

According to a fourth aspect of the invention there is hereby provided a method of treating cancer, the method comprising administering a therapeutically effective amount of a compound or pharmaceutically acceptable salt thereof according to the first aspect of the invention, or a pharmaceutical composition according to the second aspect of the invention to a patient in need thereof.

According to a fifth aspect of the invention there is hereby provided a use of a compound or pharmaceutically acceptable salt thereof according the first aspect of the invention, or a pharmaceutical composition according to the second aspect of the invention in a method of treating cancer.

According to a sixth aspect of the invention there is hereby provided a use of a compound or pharmaceutically acceptable salt according to the first aspect of the invention, or a pharmaceutical composition according to the second aspect of the invention in the manufacture of a medicament for treating cancer.

According to a seventh aspect of the invention there is hereby provided a combination comprising a compound or pharmaceutically acceptable salt thereof according to the first aspect of the invention, or a pharmaceutical composition according to the second aspect of the invention and one or more therapeutically active agents.

According to an eighth aspect of the invention there is hereby provided a method inhibiting the G12C mutant KRAS, HRAS or NRAS protein in a subject in need thereof, wherein the method comprises administering to the subject a therapeutically effective amount of a compound or pharmaceutically acceptable salt thereof according to the first aspect of the invention, or a pharmaceutical composition according to the second aspect of the invention.

### DETAILED DESCRIPTION OF THE INVENTION

The invention therefore provides the following numbered embodiments. It will be recognized that features specified in each embodiment may be combined with other specified features to provide further embodiments of the present invention.

Embodiment 1. A compound of formula (I), or a pharmaceutically acceptable salt thereof:

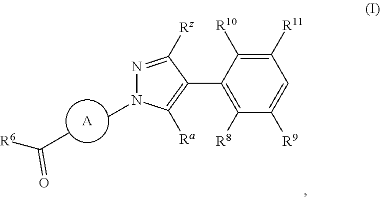

wherein:
Ring A is a 6 to 10 membered spirocyclic-heterocyclylene comprising 1 to 3 heteroatoms independently selected from N, O and S, wherein said 6 to 10 membered spirocyclic-heterocyclylene is substituted with 0 to 3 substituents R¹⁶;
G is N or CR¹²;
R^{Z} is

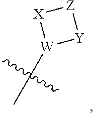

wherein
W is N;
   i) X is **—CR²₂—(CR³₂)ₙ—* or **—CR²═CR³—*, Y is **—CR⁴₂—(CR⁵₂)ₘ—*, and Z is selected from the group consisting of S(O)₂, S, S(O), O, P(O)—C₁-C₃alkyl, NR^{1N} and C(R^{1C})₂, where the * of X indicates the point of attachment to Z and the ** of X indicates the point of attachment to W, and where the * of Y indicates point of attachment to Z and the ** of Y indicates point of attachment to W, n is 0, 1 or 2 and m is 0, 1 or 2; or
   ii) X is **—CR²²—CR³═*, Y is **—CR⁴₂—(CR⁵₂)ₘ—*, and Z is selected from the group consisting of N and CR^{1C}, where the * of X indicates the point of attachment to Z and the ** of X indicates the point of attachment to W, and where the * of Y indicates point of attachment to Z and the ** of Y indicates point of attachment to W, and m is 0, 1 or 2;
R^{1N} is selected from the group consisting of H and -L^{N}_R^{2N}, preferably wherein R^{1N} is -L^{N}-R^{2N}; or
an R^{1N} group and one or two R³ groups, in combination with the atoms to which they are mutually attached, form a saturated or unsaturated 5 or 6 membered ring containing one to three heteroatoms (for example one or two heteroatoms) selected from the group consisting of N, O, S and P (preferably selected from the group consisting of N, O and S) (e.g. wherein the 5 or 6 membered ring is a 5-6 membered heterocyclyl or a 5-6 membered heteroaryl), wherein said saturated or unsaturated 5 or 6 membered ring containing one to three heteroatoms is substituted with 0 to 3 substituents R^{x}; or
an R^{1N} group and one or two R⁵ groups, in combination with the atoms to which they are mutually attached, form a saturated or unsaturated 5 or 6 membered ring containing one to three heteroatoms (for example one or two heteroatoms) selected from the group consisting of N, O, S and P (preferably selected from the group consisting of N, O and S) (e.g. wherein the 5 or 6 membered ring is a 5-6 membered heterocyclyl or a 5-6 membered heteroaryl), wherein said saturated or unsaturated 5 or 6 membered ring containing one to three heteroatoms is substituted with 0 to 3 substituents R^{x};
R^{1C}, where present, is at each occurrence independently selected from the group consisting of H and -L^{C}-R^{2C}; and/or
one or two R^{1C} group(s), and one or two R³ groups, in combination with the carbon atoms to which they are mutually attached, form a saturated or unsaturated 5 or 6 membered ring containing zero to three heteroatoms (for example one to three heteroatoms, for example one or two heteroatoms) selected from the group consisting of N, O, S and P (preferably selected from the group consisting of N, O and S) (e.g. wherein the 5 or 6 membered ring is a C₅-C₆cycloalkyl, 6 membered aryl, a 5-6 membered heterocyclyl or a 5-6 membered heteroaryl), wherein said saturated or unsaturated 5 or 6 membered ring containing zero to three heteroatoms is substituted with 0 to 3 substituents R^{x}; or
one or two R^{1C} groups, and one or two R⁵ groups, in combination with the carbon atoms to which they are mutually attached, form a saturated or unsaturated 5 or 6 membered ring containing zero to three heteroatoms (for example one to three heteroatoms, for example one or two heteroatoms) selected from the group consisting of N, O, S and P (preferably selected from the group consisting of N, O and S) (e.g. wherein the 5 or 6 membered ring is a C₅-C₆cycloalkyl, 6 membered aryl, a 5-6 membered heterocyclyl or a 5-6 membered heteroaryl), wherein said saturated or unsaturated 5 or 6 membered ring containing zero to three heteroatoms is substituted with 0 to 3 substituents R^{x}; or
two R^{1C} groups together form oxo; or
two R^{1C} groups together with the carbon atom to which they are mutually attached form a C₄-C₆cycloalkyl or a 4 to 6 membered heterocyclyl comprising 1 to 3 heteroatoms independently selected from N, O, S and P (preferably selected from the group consisting of N, O and S), said C₄-C₆cycloalkyl or 4 to 6 membered heterocyclyl being substituted with 0 to 2 substituents R^{x};
L^{N} is selected from the group consisting of a bond, C═O, C₁-C₆alkylene, SO₂, C(═O)—O*, C(═O)—C₁-C₆alkylene*, C₁-C₆alkylene-C(═O)* and C(═O)—O—C₁-C₆alkylene*, wherein * indicates the point of attachment to R^{2N}, (e.g. L^{N} is a bond);
R^{2N} is selected from the group consisting of:
   i) C₁-C₆alkyl substituted with 0 to 3 substituents R^{x}(e.g. unsubstituted C₁-C₆alkyl),
   ii) 3-10 membered heterocyclyl comprising 1 to 3 heteroatoms independently selected from N, O, S and P (preferably selected from the group consisting of N, O and S) substituted with 0 to 3 (e.g. 0 to 2) substituents R^{x},
   iii) 6 to 10 membered spirocyclic-heterocyclyl comprising 1 to 3 heteroatoms independently selected from N, O, S and P (preferably selected from the group consisting of N, O and S) substituted with 0 to 3 substituents R^{x},
   iv) hydroxyl,
   v) C₁-C₆haloalkyl,
   vi) aryl substituted with 0 to 2 substituents R^{x},
   vii) O—C₁-C₆haloalkyl,
   viii) O—C₁-C₆alkyl,
   ix) 5-6 membered heteroaryl comprising 1 to 3 (e.g. 1 or 2) heteroatoms independently selected from N, O and S substituted with 0 to 2 substituents R^{x},
   x) C₃-C₈cycloalkyl substituted with 0 to 2 substituents R^{x},
   xi) N(C₁-C₆alkyl)₂ or NH(C₁-C₆alkyl) (preferably N(C₁-C₆alkyl)₂),
   xii) CH(C₁-C₆alkylene-O—C₁-C₆alkyl)₂, and
   xiii) CN;
L^{C} is selected from the group consisting of a bond, C═O, C₁-C₆alkylene or O—C₁-C₆alkylene*, wherein * indicates the point of attachment to R^{2C}, (e.g. L^{C} is C₁alkylene);
   wherein R^{2C} is at each occurrence independently selected from the group consisting of
   i) C₁-C₆ alkyl substituted by 0 to 3 substitutents R^{x} (e.g. unsubstiuted C₁-C₆ alkyl),
   ii) hydroxyl,
   iii) 6 to 10 membered spirocyclic-heterocyclyl comprising 1 to 3 heteroatoms independently selected from N, O, S and P (preferably selected from the group consisting of N, O and S) substituted by 0 to 3 substitutents R^{x},
   iv) 5-6 membered heteroaryl comprising 1 to 3 (e.g. 1 or 2) heteroatoms independently selected from N, O and S substituted by 0 to 2 substitutents R^{x},
   v) 3-10 membered heterocyclyl comprising 1 to 3 heteroatoms independently selected from N, O, S and P (e.g. selected from N, O and S), substituted by 0 to 3 (e.g. 0 to 2) substituents R^{x} or wherein the 3-10 membered heterocyclyl is perdeuterated,
   vi) NR^{1A}R^{1B}, and
   vii)

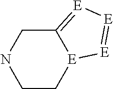

wherein E at each occasion is independently selected from CH and N substituted by 0 to 2 (e.g. 0) substitutents R^{x},
   R^{1A} and R^{1B} are each independently selected from the group consisting of H, C₁-C₆alkyl, C₁-C₆hydroxyalkyl, C₁-C₆haloalkyl, C₁-C₆alkylene-O—C₁-C₆alkyl, C₃-C₈cycloalkyl substituted by 0 to 2 substituents R^{x}, 3-10 membered heterocyclyl comprising 1 to 3 heteroatoms independently selected from N, O, S and P (preferably selected from the group consisting of N, O and S) substituted by 0 to 2 substituents R^{x}, C₁-C₆alkylene-C₃-C₈cycloalkyl substituted by 0 to 2 substituents R^{x}, C₁-C₆alkylene-3-10 membered heterocyclyl comprising 1 to 3 heteroatoms independently selected from N, O, S and P (preferably selected from the group consisting of N, O and S) substituted by 0 to 2 substituents R^{x}, SO₂-3-10 membered heterocyclyl comprising 1 to 3 heteroatoms independently selected from N, O, S and P (preferably selected from the group consisting of N, O and S) substituted by 0 to 2 substituents R^{x}, 6 to 10 membered spirocyclic-heterocyclyl comprising 1 to 3 heteroatoms independently selected from N, O, S and P (preferably selected from the group consisting of N, O and S) substituted by 0 to 2 substituents R^{x}, aryl substituted by 0 to 2 substituents R^{x}, 5-6 membered heteroaryl comprising 1 or 2 heteroatoms independently selected from N, O and S substituted by 0 to 2 substituents R^{x}, C₁-C₆alkylene-aryl substituted by 0 to 2 substituents R^{x}, C₁-C₆alkylene-5-6 membered heteroaryl comprising 1 or 2 heteroatoms independently selected from N, O and S substituted by 0 to 2 substituents R^{x}, C(═O)—C₁-C₆alkyl, C(═O)—C₁-C₆alkylene-O—C₁-C₆alkyl, and C₁-C₆alkylene-C(═O)-3-10 membered heterocyclyl comprising 1 to 3 heteroatoms independently selected from N, O, S and P (preferably selected from the group consisting of N, O and S) substituted by 0 to 2 substituents R^{x};
   R², R³, R⁴ and R⁵ are each independently selected from the group consisting of H, C₁-C₆alkyl (e.g., Me, Et, iPr), C₃-C₈cycloalkyl (preferably C₃-C₆cycloalkyl, more preferably C₃-C₅cycloalkyl, even more preferably C₃-C₄cycloalkyl), halo, C₁-C₆alkylene-O—C₁-C₆alkyl, C(═O)—C₁-C₅alkyl, C₁-C₆haloalkyl (e.g. C₁-C₆fluoroalkyl, e.g. CHF₂), hydroxyl, C₁-C₆hydroxyalkyl, NR^{1P}R^{1Q}, C₁-C₆alkylene-NR^{1P}R^{1Q}, cyano, C₁-C₆cyanoalkyl, C₁-C₆alkylene-O—C₁-C₆haloalkyl, C(═O)—NHC₁-C₅alkyl, C(═O)—N(C₁-C₅alkyl)₂, and C(═O)—O—C₁-C₅alkyl, wherein R^{1P} and R^{1Q} are each independently selected from the group consisting of H, C(═O)—C₁-C₆alkyl, C₁-C₆alkyl, C₁-C₆alkylene-O—C₁-C₆alkyl, C₁-C₆hydroxyalkyl or wherein R^{1P} and R^{1Q} together with the nitrogen atom to which they are mutually attached form a 4 to 6 membered heterocyclyl comprising 1 or 2 heteroatoms independently selected from N, O, S;
preferably R², R³, R⁴ and R⁵ are each independently selected from the group consisting of H, C₁-C₆alkyl (e.g., Me, Et, iPr), C₃-C₈cycloalkyl (preferably C₃-C₆cycloalkyl, more preferably C₃-C₅cycloalkyl, even more preferably C₃-C₄cycloalkyl), C₁-C₆alkylene-O—C₁-C₆alkyl, C(═O)—C₁-C₅alkyl, C₁-C₆haloalkyl (e.g. C₁-C₆fluoroalkyl, e.g. CHF₂), C₁-C₆hydroxyalkyl, cyano, and C₁-C₆cyanoalkyl; and/or
   i) an R² group and an R⁴ group in combination form a bridging group;
   ii) an R² group and an R⁵ group in combination form a bridging group;
   iii) an R³ group and an R⁴ group in combination form a bridging group; or
   iv) an R³ group and an R⁵ group in combination form a bridging group;
wherein the bridging group forms a C₄-C₆cycloalkyl, or a 4 to 6 membered heterocyclyl comprising 1 to 3 heteroatoms independently selected from the group consisting of N, O, S and P (preferably selected from the group consisting of N, O and S), wherein the C₄-C₆cycloalkyl or 4 to 6 membered heterocyclyl are each substituted with 0 to 3 substituents R^{x}; or
   i) an R² group and an R³ group in combination with the carbon atoms to which they are mutually attached form a ring; and/or
   ii) an R⁴ group and an R⁵ group in combination with the carbon atoms to which they are mutually attached form a ring;
wherein the ring is a C₄-C₆cycloalkyl, or a 4 to 6 membered heterocyclyl comprising 1 to 3 heteroatom independently selected from the group consisting of N, O, S and P (preferably selected from the group consisting of N, O and S), wherein the C₄-C₆cycloalkyl or 4 to 6 membered heterocyclyl are each substituted with 0 to 3 substituents R^{x}; and/or
   i) two R² groups in combination form an oxo or in combination with the carbon atom to which they are mutually attached form a ring;
   ii) two R³ groups in combination form an oxo or in combination with the carbon atom to which they are mutually attached form a ring;
   iii) two R⁴ groups in combination form an oxo or in combination with the carbon atom to which they are mutually attached form a ring; or
   iv) two R⁵ groups in combination form an oxo or in combination with the carbon atom to which they are mutually attached form a ring;
   wherein the ring is a C₃-C₆cycloalkyl or a 3 to 6 membered heterocyclyl comprising 1 or 2 heteroatoms independently selected from the group consisting of N, O, S and P (preferably selected from the group consisting of N, O and S), wherein the C₃-C₆cycloalkyl or 3 to 6 membered heterocyclyl is substituted with 0 to 3 substituents R^{x};
   each R^{x} is independently selected from a) C₁-C₃alkyl (e.g., Me, Et, iPr), b) halo (preferably fluoro), c) C(═O)—C₁-C₃alkyl, d) C(═O)—C₁-C₃hydroxyalkyl, e) cyano, f) hydroxyl, g) amino, h) oxo, i) O—C₁-C₃alkyl, j) C₁-C₃hydroxyalkyl, k) C₁-C₃haloalkyl, l) O—C₁-C₃haloalkyl, m) COOH, n) SO₂—C₁-C₃alkyl, o) C₁-C₃alkylene-O—C₁-C₃alkyl, p) C₃-C₆cycloalkyl substituted by 0 to 2 (preferably 0) substituents selected from the group consisting of CH₃, OH, OMe, F and CN, q) 3 to 6 membered heterocyclyl comprising 1 to 3 heteroatoms (preferably 1 or 2 heteroatoms) independently selected from the group consisting of N, O and S (preferably selected from the group consisting of N and O) substituted by 0 to 2 (preferably 0) substituents selected from the group consisting of CH₃, OH, OMe, F and CN, r) NR^{Xa}R^{Xb}, s) C(═O)—NR^{Xa}R^{Xb}, and t) deuterium;
   wherein R^{Xa} and R^{Xb} are independently selected from the group consisting of H, C(═O)—C₁-C₆alkyl, SO₂—C₁-C₃alkyl, C₂-C₄haloalkyl, C₂-C₄alkylene-O—C₁-C₃alkyl, C₁-C₃alkyl and 3 to 6 membered heterocyclyl comprising 1 to 3 heteroatoms (preferably 1 or 2 heteroatoms) independently selected from the group consisting of N, O and S (preferably selected from the group consisting of N and O) (preferably wherein R^{Xa} and R^{Xb} are independently selected from the group consisting of H and C₁-C₃alkyl);
   R⁶ is CR^{7a}═CR^{7b}₂, C≡CR^{7b}, or CR^{7c}₃;
   R^{7a}, where present, is H or fluoro (preferably R^{7a} where present is H);
   each R^{7b} is independently selected from the group consisting of H, halo (preferably fluoro or chloro) and C(R^{7d})₃ wherein each R^{7d} is independently selected from the group consisting of H, halo (preferably fluoro or chloro), O—C₁-C₆alkyl, C₁-C₆alkyl, hydroxyl, and NR^{7e}R^{7f}, wherein R^{7e} and R^{7f} are each H or C₁-C₆alkyl, or wherein R^{7e} and R^{7f} together with the nitrogen atom to which they are mutually attached form a 3 to 8 membered heterocyclyl comprising 1 to 3 heteroatoms each independently selected from the group consisting of N, O, S and P, with at least one heteroatom being nitrogen, (preferably selected from the group consisting of N, O and S with at least one heteroatom being N) with the proviso that if one R^{7d} substituent is selected from the group consisting of O—C₁-C₆ alkyl, hydroxyl or NR^{7e}R^{7f}, the other two R^{7d} substituents are both H;
   one R^{7c} is selected from the group consisting of H, halo (preferably fluoro or chloro) and C₁-C₆alkyl (preferably wherein one R^{7C} is H) and the other two R^{7c} groups in combination with the carbon atom to which they are mutually attached form a 3 membered heterocyclyl comprising 1 heteroatom selected from the group consisting of N and O (preferably wherein the heteroatom is O);
   R⁸ is H, halo (preferably chloro), O—C₁-C₃alkyl (preferably OMe), C₃-C₄cycloalkyl,

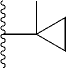 [SMILES: *C1(C)CC1]

or C(Rea)₃, wherein each R^{8a} is independently selected from the group consisting of H, C₁-C₃alkyl, and halo (preferably fluoro), preferably wherein each R^{8a} is H (e.g. wherein each R^{8a} is D), and
R⁹ is H, halo (preferably fluoro or chloro), NH₂, hydroxyl, C₃-C₄cycloalkyl or C(R^{9a})₃, wherein each R^{9a} is independently selected from the group consisting of H, C₁-C₃alkyl, and halo (preferably fluoro), preferably wherein each R^{9a} is H (e.g. wherein each R^{9a} is D), or R⁸ and R⁹ together with the aryl ring to which they are mutually attached form

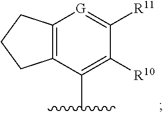

R¹⁰ is selected from the group consisting of H, halo, NH₂, C₁-C₃alkyl (preferably Me), and hydroxyl and R¹¹ is selected from the group consisting of H, halo, NH₂, hydroxyl and C₁-C₃alkyl (preferably wherein R¹¹ is H or hydroxyl), or
R¹⁰ and R¹¹ are joined together to form, in combination with the 6 membered aryl or heteroaryl to which they are mutually attached, a 9 or 10 (preferably 9) membered fused bicyclic aryl or heteroaryl group containing 1 to 3 (preferably 2) heteroatoms independently selected from the group consisting of N, O, and S (preferably wherein the heteroatom(s) are independently selected from the group consisting of N and O, more preferably wherein each heteroatom is N), wherein said fused bicyclic heteroaryl group is substituted with 0 to 3 substituents independently selected from the group consisting of C₁-C₆alkyl (preferably methyl), NH₂, R¹⁴, R¹⁵, R¹⁷, R¹⁸, R¹⁹ and R²⁰;
R¹² is H, halo (preferably fluoro) or methyl, preferably R¹² is H;
R^{a} is H, CN or C(R¹³)₃,
each R¹³ is independently selected from the group consisting of H, deuterium, halo (preferably fluoro), C₁-C₃alkyl and hydroxyl, provided that no more than one R¹³ is hydroxyl,
or two R¹³ substituents in combination with the carbon atom to which they are mutually attached form a C₃-C₅cycloalkyl or a 3 to 5 membered heterocyclyl comprising 1 to 3 (preferably 1) heteroatoms each independently selected from the group consisting of N, O, S and P (preferably selected from the group consisting of N, O and S), preferably wherein the two R¹³ substituents in combination with the carbon atom to which they are mutually attached form a C₃cycloalkyl, and the third R₁₃ substituent is H, halo (preferably fluoro), C₁-C₃alkyl or hydroxyl (preferably wherein the third R₁₃ substituent is H),
R¹⁴ is selected from the group consisting of H and C₁-C₃alkyl;
R¹⁵, R¹⁷, R¹⁸, R¹⁹ and R²⁰ are each independently selected from the group consisting of H, halo, C₁-C₃alkyl and NH₂; and
each R¹⁶ group is independently selected from the group consisting of C₁-C₃alkyl, cyano, halo (e.g. fluoro), hydroxyl, O—C₁-C₃alkyl, C₁-C₃haloalkyl (e.g. C₁-C₃fluoroalkyl), C₁-C₃hydroxyalkyl, and C₁-C₃cyanoalkyl.

Embodiment 1a. A compound according to Embodiment 1 wherein the compound of formula (I) is a compound according to formula (1i)

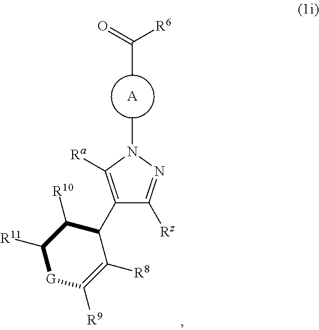

wherein G, Ring A, R^{a}, R^{Z}, R⁶, R⁸, R⁹, R¹⁰ and R¹¹ are as defined in Embodiment 1, or a pharmaceutically acceptable salt thereof.

Embodiment 2. A compound according to Embodiment 1 or Embodiment 1a wherein Ring A is a 6 to 9 membered spirocyclic-heterocyclylene comprising 1 or 2 heteroatoms independently selected from N and O, wherein said 6 to 9 membered spirocyclic-heterocyclylene is substituted with 0 or 1 R¹⁶ substituents, or a pharmaceutically acceptable salt thereof.

Embodiment 3. A compound according to Embodiment 2 wherein Ring A is a 7 to 9 (preferably 7 or 8) membered spirocyclic-heterocyclylene comprising 1 heteroatom which is N, wherein said spirocyclic-heterocyclylene is substituted with 0 to 1 C₁-C₃alkyl (preferably methyl) substituents, or a pharmaceutically acceptable salt thereof.

Embodiment 3a. A compound according to any one of the preceding Embodiments wherein the ring atom in Ring A which is directly attached to the C(═O)R⁶ group is a Nitrogen, or a pharmaceutically acceptable salt thereof.

Embodiment 4. A compound according to Embodiment 1 or Embodiment 1a wherein Ring A is selected from the group consisting of

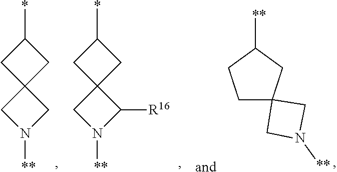

wherein * denotes the point of attachment to the pyrazole ring and ** denotes the point of attachment to C(═O)R⁶, and wherein R¹⁶ is selected from the group consisting of C₁-C₃alkyl (for example Me), C₁-C₃fluoroalkyl, C₁-C₃hydroxyalkyl (for example CH₂OH) and C₁-C₃cyanoalkyl (for example CH₂CN), or a pharmaceutically acceptable salt thereof.

Embodiment 5. A compound according to Embodiment 4, wherein Ring A is

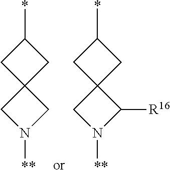

wherein * denotes the point of attachment to the pyrazole ring and ** denotes the point of attachment to —C(═O)R⁶, and wherein R¹⁶ is C₁-C₃alkyl, or a pharmaceutically acceptable salt thereof.

Embodiment 6. A compound according to any one of the preceding Embodiments, wherein R¹⁶ is methyl, or a pharmaceutically acceptable salt thereof.

Embodiment 7. A compound according to any one of the preceding Embodiments, wherein G is CR¹², or a pharmaceutically acceptable salt thereof.

Embodiment 8. A compound according to Embodiment 7 wherein R¹² is H, or a pharmaceutically acceptable salt thereof.

Embodiment 9. A compound according to any one of the preceding Embodiments wherein X is **—CR²₂—(CR³₂)ₙ—*, Y is **—CR⁴₂—(CR⁵₂)ₘ—*, and Z is selected from the group consisting of S(O)₂, S, S(O), O, NR^{1N} and C(R^{1C})₂, where the * of X indicates the point of attachment to Z and the ** of X indicates the point of attachment to W, and where the * of Y indicates point of attachment to Z and the ** of Y indicates point of attachment to W, n is 0, 1 or 2 and m is 0, 1 or 2, or a pharmaceutically acceptable salt thereof.

Embodiment 10. A compound according to Embodiment 9 wherein n is 0 or 1, or a pharmaceutically acceptable salt thereof.

Embodiment 11. A compound according to Embodiment 10 wherein n is 1, or a pharmaceutically acceptable salt thereof.

Embodiment 12. A compound according to any one of the preceding Embodiments wherein m is 1, or a pharmaceutically acceptable salt thereof.

Embodiment 13. A compound according to Embodiment 10 wherein n and m are both 0 or both 1, or a pharmaceutically acceptable salt thereof.

Embodiment 14. A compound according to Embodiment 13 wherein n and m are both 1, or a pharmaceutically acceptable salt thereof.

Embodiment 15. A compound according to any one of Embodiments 8 to 14 wherein Z is NR^{1N} or C(R^{1C})₂, or a pharmaceutically acceptable salt thereof.

Embodiment 16. A compound according to Embodiment 15 wherein Z is NR^{1N} or CHR^{1C}, or a pharmaceutically acceptable salt thereof.

Embodiment 17. A compound according to any one of the preceding Embodiments wherein

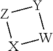

is selected from the group consisting of:

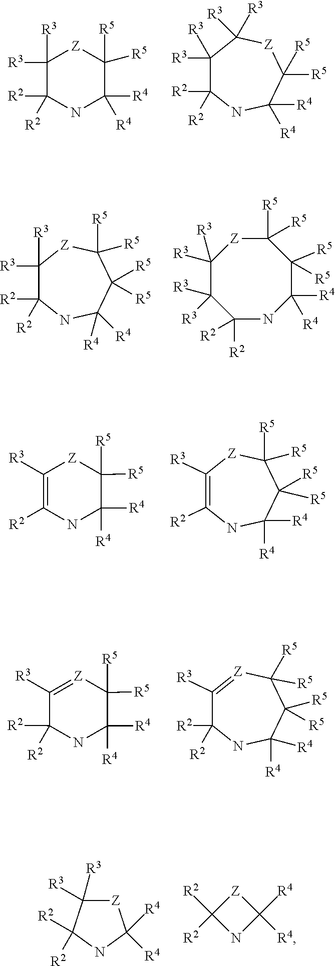

or a pharmaceutically acceptable salt thereof.

Embodiment 18. A compound according to Embodiment 17, wherein

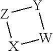

is selected from the group consisting of:

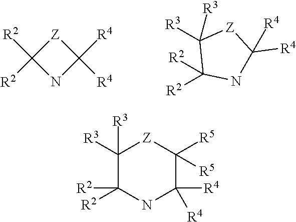

preferably wherein

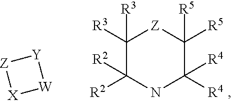

or a pharmaceutically acceptable salt thereof.

Embodiment 19. A compound according to any one of the preceding Embodiments wherein R⁶ is CR^{7a}═C(R^{7b})₂, or a pharmaceutically acceptable salt thereof.

Embodiment 20. A compound according to any one of the preceding Embodiments wherein R^{7a} is H, or a pharmaceutically acceptable salt thereof.

Embodiment 21. A compound according to Embodiment 19 or Embodiment 20 wherein each R^{7b} is independently selected from the group consisting of H, halo (preferably chloro), or a pharmaceutically acceptable salt.

Embodiment 22. A compound according to Embodiment 21 wherein each R^{7b} is H or wherein one R^{7b} is H and one R^{7b} is halo (preferably chloro) or a pharmaceutically acceptable salt thereof.

Embodiment 23. A compound according to Embodiment 22 wherein each R^{7b} is H, or a pharmaceutically acceptable salt thereof.

Embodiment 24. A compound according to any one of the preceding Embodiments wherein R¹⁰ and R¹¹ are joined together, in combination with the 6 membered aryl or heteroaryl to which they are mutually attached, to form a fused bicyclic aryl or heteroaryl group selected from the group consisting of:

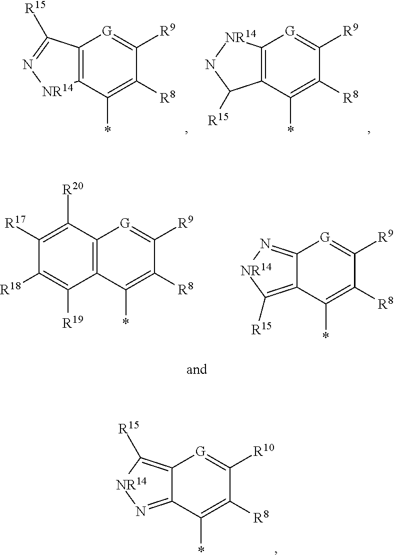

wherein:
G, R⁸, R⁹, R¹⁴, R¹⁵, R¹⁷, R¹⁸, R¹⁹ and R²⁰ are as defined in any one of the preceding Embodiments, and wherein * denotes where the fused bicyclic heteroaryl group is attached to the remainder of the molecule, or a pharmaceutically acceptable salt thereof.

Embodiment 25. A compound according to Embodiment 24 wherein R¹⁰ and R¹¹ are joined together with the 6 membered aryl or heteroaryl to which they are attached to form a fused bicyclic aryl or heteroaryl group selected from the group consisting of

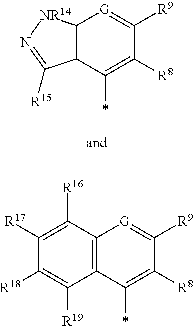

or a pharmaceutically acceptable salt thereof.

Embodiment 26. A compound according to Embodiment 25 wherein R¹⁰ and R¹¹ are joined together with the 6 membered aryl or heteroaryl to which they are attached to form the fused bicyclic heteroaryl group

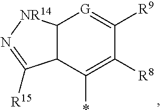

or a pharmaceutically acceptable salt thereof, for example, wherein the compound is of formula (1ii),

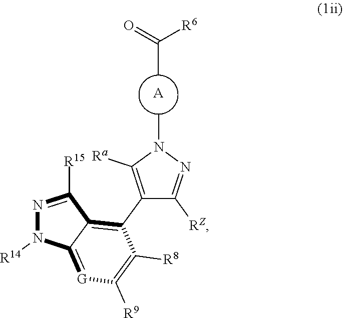

wherein G, Ring A, R^{a}, R^{Z}, R⁶, R⁸, R⁹, R¹⁴ and R¹⁵ are as defined in any one of the preceding Embodiments, or a pharmaceutically acceptable salt thereof.

Embodiment 27. A compound according to any one of Embodiments 24 to 26 wherein R¹⁴, where present, is H, or a pharmaceutically acceptable salt thereof.

Embodiment 28. A compound according to any one of Embodiments 24 to 27 wherein R¹⁵, where present, is H or NH₂, or a pharmaceutically acceptable salt thereof.

Embodiment 29. A compound according to Embodiment 28 wherein R¹⁵, where present, is H, or a pharmaceutically acceptable salt thereof.

Embodiment 30. A compound according to any one of the preceding Embodiments wherein R^{a} is CN or C(R¹³)₃, or a pharmaceutically acceptable salt thereof.

Embodiment 31. A compound according to Embodiment 30 wherein:
i) each R¹³ is independently selected from fluoro, H and deuterium (for example wherein each R¹³ is H, or wherein each R¹³ is deuterium),
ii) wherein one R¹³ is H, and the other two R¹³ groups in combination form C₃ cycloalkyl, or
iii) R^{a} is CN,
or a pharmaceutically acceptable salt thereof.

Embodiment 32. A compound according to Embodiment 31 wherein i) each R¹³ is H, or ii) each R¹³ is deuterium, or a pharmaceutically acceptable salt thereof.

Embodiment 33. A compound according to Embodiment 32 wherein each R¹³ is H, or a pharmaceutically acceptable salt thereof.

Embodiment 34. A compound according to any one of the preceding Embodiments wherein R⁸ is halo (e.g. chloro), methyl, H or OMe, or a pharmaceutically acceptable salt thereof.

Embodiment 35. A compound according to Embodiment 34 wherein R⁸ is chloro or methyl, or a pharmaceutically acceptable salt thereof.

Embodiment 36. A compound according to any one of the preceding Embodiments wherein R⁹ is H, methyl or halo (e.g. chloro or fluoro), or a pharmaceutically acceptable salt thereof.

Embodiment 37. A compound according to Embodiment 36 wherein R⁹ is methyl or chloro, or a pharmaceutically acceptable salt thereof.

Embodiment 38. A compound according to any one of the preceding Embodiments wherein each R² is independently selected from the group consisting of H, C₁-C₃alkyl, C₃-C₄cycloalkyl, C₁-C₃alkylene-O—C₁-C₃alkyl, C₁-C₃haloalkyl and cyano, or
wherein, where present, two R² groups in combination with the carbon atom to which they are mutually attached form a C₃-C₆cycloalkyl or a 3 to 6 membered heterocyclyl comprising 1 or 2 heteroatoms independently selected from N and O, wherein the C₃-C₆cycloalkyl or 3 to 6 membered heterocyclyl is substituted with 0 to 2 substituents (preferably 0 or 1 substituents) R^{x}, or a pharmaceutically acceptable salt thereof.

Embodiment 39. A compound according to Embodiment 38 wherein each R² is independently selected from the group consisting of H, C₁-C₃ alkyl, C₁-C₃haloalkyl and C₁-C₃alkylene-O—C₁-C₃alkyl, or
wherein, where present, two R² groups in combination with the carbon atom to which they are mutually attached form a C₄-C₆cycloalkyl or 4 to 6 membered heterocyclyl comprising 1 or 2 heteroatoms independently selected from the group consisting of N and O, wherein the C₄-C₆cycloalkyl or 4 to 6 membered heterocyclyl is unsubstituted or substituted with C(═O)—CH₃, or a pharmaceutically acceptable salt thereof.

Embodiment 40. A compound according to Embodiment 39 wherein each R² is independently selected from the group consisting of H, C₁-C₃ alkyl, C₁-C₃fluoroalkyl and C₁-C₃alkylene-O—C₁-C₃alkyl or wherein, where present, two R² groups in combination with the carbon atom to which they are mutually attached form a C₄-C₅cycloalkyl or 4 to 6 membered heterocyclyl comprising 1 heteroatom which is N or O, wherein the 4 to 6 membered heterocyclyl is unsubstituted or substituted with C(═O)—CH₃,
for example wherein each R² is independently selected from the group consisting of C₁-C₃ alkyl, C₁-C₃fluoroalkyl or C₁-C₃alkylene-O—C₁-C₃alkyl or wherein, where present, two R² groups in combination with the carbon atom to which they are mutually attached form a C₄-C₅cycloalkyl, or a pharmaceutically acceptable salt thereof.

Embodiment 41. A compound according to any one of the preceding Embodiments wherein each R⁴ is independently selected from H and C₁-C₃alkyl (for example, Me), or a pharmaceutically acceptable salt thereof.

Embodiment 42. A compound according to Embodiment 41 wherein each R⁴ is H, or a pharmaceutically acceptable salt thereof.

Embodiment 43. A compound according to any one of the preceding Embodiments wherein each R³ is independently H, halo (e.g. fluoro) or C₁-C₃alkyl, or a pharmaceutically acceptable salt thereof.

Embodiment 44. A compound according to Embodiment 43 wherein each R³ is H, or a pharmaceutically acceptable salt thereof.

Embodiment 45. A compound according to any one of the preceding Embodiments wherein each R⁵ is independently selected from H and Me (preferably wherein each R⁵ is H), or a pharmaceutically acceptable salt thereof.

Embodiment 46. A compound according to any one of the preceding Embodiments wherein R^{1A} and R^{1B}, where present, are independently selected from the group consisting of: i) C₁-C₆alkyl,
ii) C(═O)—C₁-C₆alkyl,
iii) C(═O)—C₁-C₆alkylene-O—C₁-C₆alkyl,
iv) C₁-C₆hydroxyalkyl,
v) C₁-C₆alkylene-O—C₁-C₆alkyl,
vi) 3-10 membered heterocyclyl comprising 1 to 3 heteroatoms independently selected from N, O and S substituted by 0 to 2 substituents R^{x},
vii) SO₂-3-10 membered heterocyclyl comprising 1 to 3 heteroatoms independently selected from N, O and S said 3-10 membered heterocyclyl comprising 1 to 3 heteroatoms independently selected from N, O and S being substituted by 0 to 2 substituents R^{x}, and
viii) C₁-C₆alkylene-C(═O)-3-10 membered heterocyclyl, said 3-10 membered heterocyclyl comprising 1 to 3 heteroatoms independently selected from N, O and S being substituted by 0 to 2 substituents R^{x}, or a pharmaceutically acceptable salt thereof.

Embodiment 47. A compound according to any one of the preceding Embodiments wherein each R^{x} is independently selected from the group consisting of C₁-C₃alkylene-O—C₁-C₃alkyl, C₁-C₃alkyl (e.g., Me, Et, iPr), halo (preferably fluoro), oxo, hydroxyl, O—C₁-C₃alkyl and 3 to 6 membered heterocyclyl comprising 1 to 3 heteroatoms (preferably 1 or 2 heteroatoms) independently selected from the group consisting of N, O and S and C₁-C₃hydroxyalkyl,
preferably wherein each R^{x} is independently selected from the group consisting of C₁-C₃alkylene-O—C₁-C₃alkyl, C₁-C₃alkyl (e.g., Me, Et, iPr), halo (preferably fluoro), 3 to 6 membered heterocyclyl comprising 1 to 3 heteroatoms (preferably 1 or 2 heteroatoms) independently selected from the group consisting of N, O and S, and oxo,
or a pharmaceutically acceptable salt thereof.

Embodiment 48. A compound according to any one of the preceding Embodiments wherein the compound is a compound of formula (II) or (IIa)

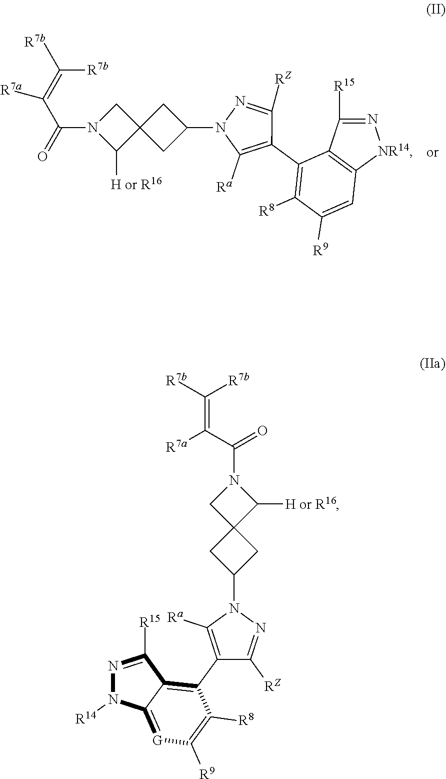

wherein R^{a} is C(R¹³)₃, wherein each R¹³ is independently as defined in any one of the preceding Embodiments, preferably wherein each R¹³ is independently selected from H, deuterium and C₁-C₃alkyl, most preferably wherein each R¹³ is H,
R^{7a}, R^{7b}, R⁸, R⁹, R¹⁴, R¹⁵ and R¹⁶ are as defined in any one of the previous Embodiments, and
R^{Z} is selected from the group consisting of:

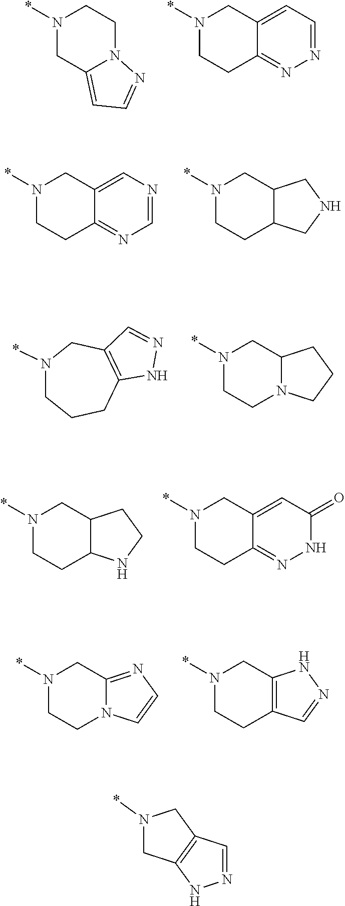

wherein * indicates the point of attachment to the remainder of the molecule,
and wherein any of the above R^{Z} groups are substituted with 0 to 3 (preferably 0 to 2) substituents independently selected from the group consisting of C₁-C₃alkyl, C₁-C₃haloalkyl (preferably C₁-C₃fluoroalkyl), oxo (═O), C(═O)—C₁-C₃alkyl, cyano, and halo (preferably fluoro),
or R^{Z} is selected from

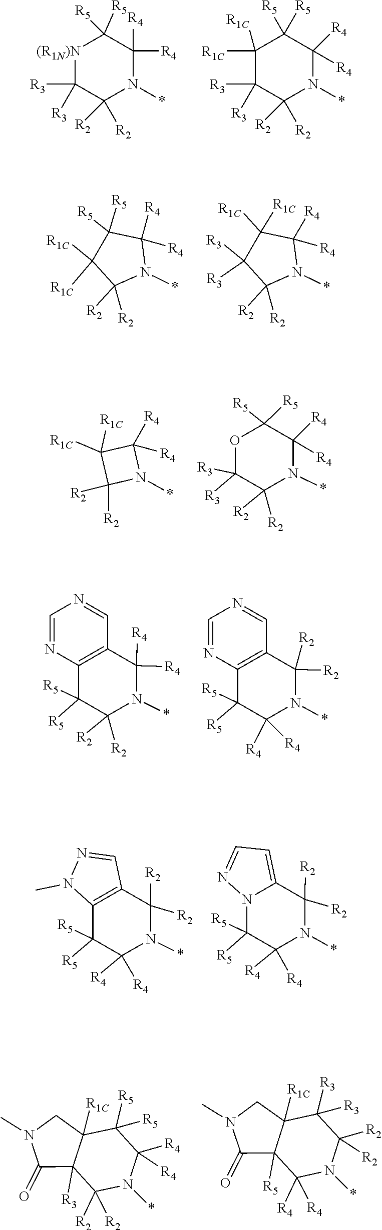

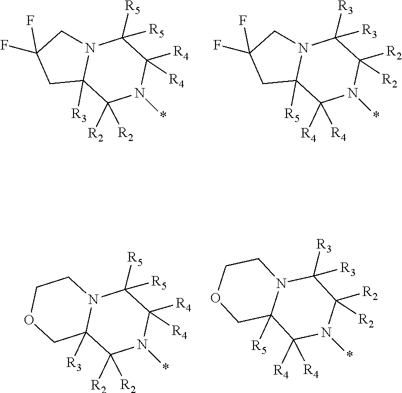

wherein R², R³, R⁴, R⁵, R^{1C} and R^{1N} are as defined in any one of the preceding Embodiments,
or a pharmaceutically acceptable salt thereof.

(The skilled person would naturally understand that oxo (═O) can only be attached to an sp³ ring carbon, and not an sp² ring carbon, given that sp² ring carbons lack the necessary valency.)

Purely by way of an example, when R^{Z} is

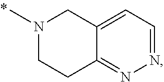

substituted with 0 substituents, formula (II) is

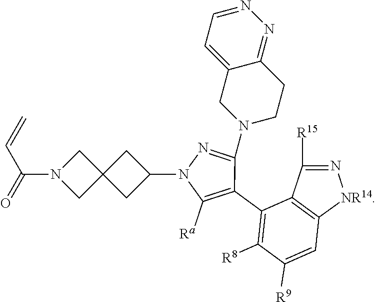

Embodiment 49. A compound according to any one of the preceding Embodiments wherein the compound according to formula (I) is a compound according to formula (III) or (IIIa)

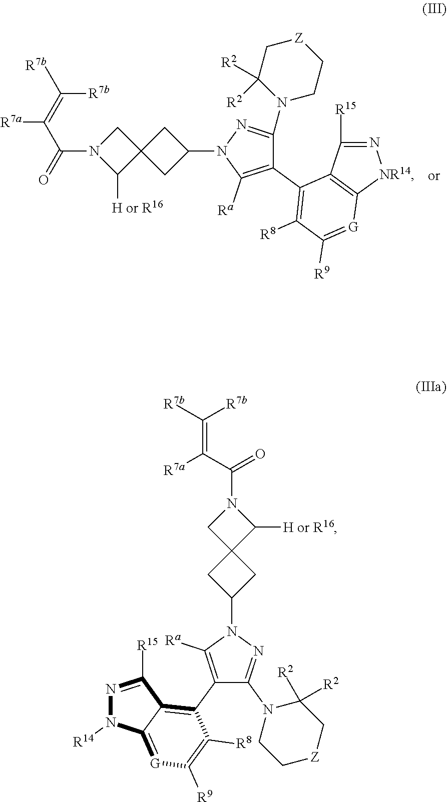

wherein G, R^{a}, R^{7a}, R^{7b}, R⁸, R⁹, R¹⁴, R¹⁵ and R¹⁶ are as defined in any one of the preceding Embodiments,
preferably wherein G is CH, preferably wherein R^{a} is methyl, preferably wherein R^{7a} is H, preferably wherein both R^{7b} are H, preferably wherein R⁸ is Cl or Me, preferably wherein R⁹ is Cl or Me,
preferably wherein R¹⁴ is H, preferably wherein R¹⁵ is H or NH₂, more preferably wherein R¹⁵ is H, preferably wherein the group labelled as “H or R¹⁶” is H,
Z is selected from the group consisting of S, S(O), S(O)₂, NR^{1N} and C(R^{1C})₂, and R^{1N} and R^{1C} are as defined in any one of the preceding Embodiments, and and
wherein each R² is independently selected from the group consisting of C₁-C₃ alkyl, C₁-C₃fluoroalkyl or C₁-C₃alkylene-O—C₁-C₃alkyl or wherein, where present, two R² groups in combination with the carbon atom to which they are mutually attached form a C₄-C₅cycloalkyl or 4 to 6 membered heterocyclyl comprising 1 heteroatom which is N or O, wherein the C₄-C₅cycloalkyl or 4 to 6 membered heterocyclyl is unsubstituted or substituted with C(═O)—CH₃,
preferably wherein each R² is independently selected from the group consisting of C₁-C₂alkyl and C₁-C₂alkylene-O—C₁-C₂alkyl, more preferably each R² is independently selected from the group consisting of C₁-C₂alkyl and C₂alkylene-O—C₁alkyl,
or a pharmaceutically acceptable salt thereof.

Embodiment 50. A compound according to any one of the preceding Embodiments wherein Z is NR^{1N}, and wherein R^{1N} is selected from the group consisting of:
C(═O)—C₁-C₆alkyl,
C₁-C₆hydroxyalkyl,
3-10 membered heterocyclyl comprising 1 to 3 heteroatoms independently selected from the group consisting of N, O and S,
C(═O)—O—C₁-C₆alkylene-O—C₁-C₆alkyl,
C₁-C₆alkylene-3-10 membered heterocyclyl comprising 1 to 3 heteroatoms independently selected from the group consisting of N, O and S,
CH[C₁-C₃alkylene-O—C₁-C₃alkyl]₂,
C₁-C₆alkyl,
5-6 membered heteroaryl comprising 1 to 3 (e.g. 1 or 2) heteroatoms which are independently selected from the group consisting of N, O and S, said 5-6 membered heteroaryl optionally being substituted with C₁-C₃alkyl,
C₁-C₆alkylene-5-6 membered heteroaryl comprising 1 to 3 heteroatoms which are independently selected from the group consisting of N, O and S, said 5-6 membered heteroaryl being optionally substituted with C₁-C₃alkyl,
C₁-C₆alkylene-O—C₁-C₆alkyl,
C₁-C₆haloalkyl,
6-10 membered spirocyclic-heterocyclyl comprising 1 to 3 heteroatoms independently selected from N, O and S,
C(═O)—C₁-C₆alkylene-O—C₁-C₆alkylene,
C(═O)-3-10 membered heterocyclyl comprising 1 to 3 independently heteroatoms independently selected from the group consisting of N, O and S,
C₁-C₆alkylene-aryl,
C₁-C₆alkylene-O—C₁-C₆haloalkyl,
C(═O)—O—C₁-C₆alkyl,
C₁-C₆alkylene-3-10 membered heterocyclyl comprising 1 to 3 heteroatoms independently selected from the group consisting of N, O and S said 3-10 membered heterocyclyl being substituted with oxo,
3-10 membered heterocyclyl comprising 1 to 3 heteroatoms independently selected from the group consisting of N, O and S, said 3-9 membered heterocyclyl being optionally substituted by C₁-C₃alkyl,
C₁-C₆alkylene-C(═O)—O—C₁-C₆alkyl,
SO₂—C₁-C₆alkyl,
C(═O)—C₃-C₈cycloalkyl,
C(═O)—N(C₁-C₆alkyl)₂,
C(═O)—C₁-C₆haloalkyl, and
C₁-C₆alkylene-C₃-C₈cycloalkyl, said C₃-C₈cycloalkyl being substituted with hydroxyl,
or a pharmaceutically acceptable salt thereof.

Embodiment 51. A compound according to any one of Embodiments 1, 1a and 50 wherein R^{1N} is selected from the group consisting of
C(═O)—C₁alkyl,
C₂hydroxyalkyl,
6 membered heterocyclyl comprising 1 heteroatom which is O,
5 membered heterocyclyl comprising 1 heteroatom which is O,
C₄hydroxyalkyl,
4 membered heterocyclyl comprising 1 heteroatom which is O,
C(═O)—C₁alkylene-O—C₁alkyl,
C(═O)—O—C₂alkylene-O—C₁alkyl,
C₁alkylene-7 membered heterocyclyl comprising 2 heteroatoms which are each O,
CH—[(C₂alkylene)-O—(C₁alkyl)]₂,
C₃alkyl,
C₁alkylene-5 membered heteroaryl comprising 3 heteroatoms which are all N, said 5 membered heteroaryl being substituted with C₁alkyl,
5 membered heteroaryl comprising 2 heteroatoms which are both N, said 5 membered heteroaryl being substituted with C₁alkyl,
C₂alkylene-O—C₁alkyl,
7 membered spirocyclic-heterocyclyl comprising 1 heteroatom which is O,
C₂haloalkyl,
C₄hydroxyalkyl,
C(═O)-5 membered heterocyclyl comprising 1 heteroatom which is O,
C₁alkylene-aryl,
C₁alkylene-6 membered heterocyclyl comprising 2 heteroatoms which are both O,
C₂-alkylene-O—C₁haloalkyl,
C(═O)—O—C₁alkyl,
C₁alkylene-5 membered heterocyclyl comprising 1 heteroatom which is O,
4 membered heterocyclyl comprising 1 heteroatom which is O, said 4 membered heterocyclyl being substituted by C₁alkyl,
C₃alkylene-C(═O)—O—C₁alkyl,
6 membered heteroaryl comprising 2 heteroatoms which are both N,
SO₂—C₁alkyl,
C(═O)—C₃cycloalkyl,
C(═O)—N(C₁alkyl)₂,
C(═O)—C₁haloalkyl,
C₁alkylene-C₃cycloalkyl, said C₃cycloalkyl being substituted with hydroxyl,
C₁alkylene-7 membered heterocyclyl comprising 2 heteroatoms which are each O,
C₁alkyl, and
C₂alkylene-6 membered heterocyclyl comprising 2 heteroatoms which are N and O, said 6 membered heterocyclyl being substituted by oxo,
or a pharmaceutically acceptable salt thereof.

Embodiment 52. A compound according to any one of Embodiments 1, 1a, 50 and 51, wherein R^{1N} is selected from the group consisting of C(═O)—CH₃,

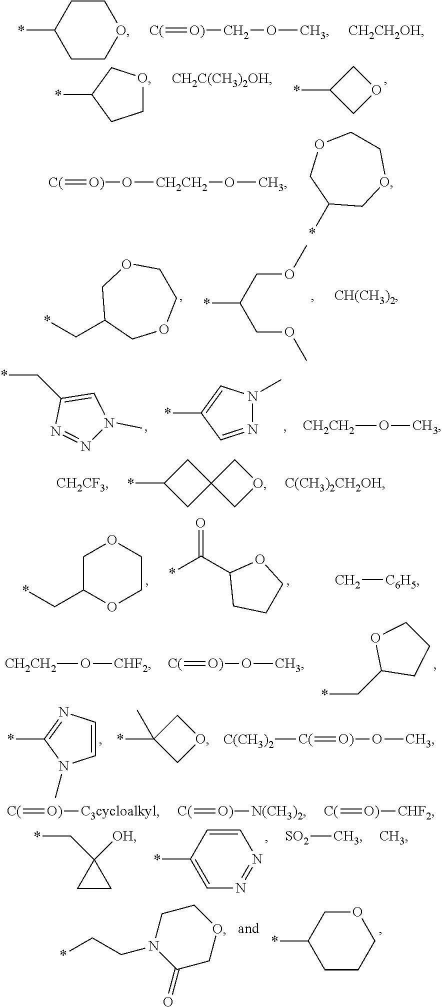

wherein * indicates the point of attachment to the remainder of the molecule, or a pharmaceutically acceptable salt thereof.

By way of example, where R^{1N} is

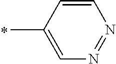

the compound can be expressed by the following formula:

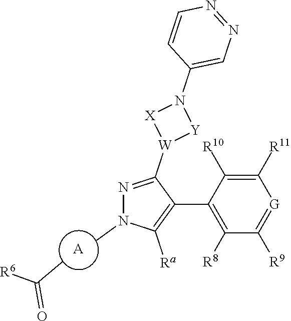

By way of a further example, where R^{1N} is

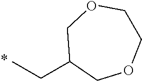

the compound can be expressed by the following formula

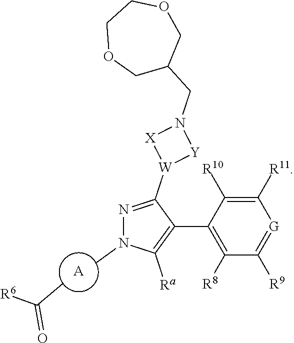

Embodiment 53. A compound according to any one of Embodiments 1 to 49 wherein Z is CHR^{1C}, and wherein R^{1C} is selected from the group consisting of
a) H,
b) N(C₁-C₆alkyl)-C(═O)—C₁-C₆alkyl,
c) N(C₁-C₆alkyl)-C(═O)—C₁-C₆alkylene-O—C₁-C₆alkyl,
d) N(C₁-C₆alkylene-O—C₁-C₆alkyl)₂,
e) C₀-C₆alkylene-N(C₁-C₆alkylene-O—C₁-C₆alkyl)(C₁-C₆hydroxyalkyl),
f) N(C₁-C₆alkyl)-3-10 membered heterocyclyl, said 3-10 membered heterocyclyl comprising 1 to 3 heteroatoms independently selected from the group consisting of N, O, S and P,
g) O—C₁-C₆alkylene-3-10 membered heterocyclyl, said 3-10 membered heterocyclyl comprising 1 to 3 heteroatoms selected from the group consisting of N, O, S and P,
h) C₁-C₆alkylene-N(C₁-C₆alkyl)-C₁-C₆alkylene-C(═O)-3-10 membered heterocyclyl, said 3-10 membered heterocyclyl comprising 1 to 3 heteroatoms selected from the group consisting of N, O, S and P,
i) C₁-C₆alkylene-N(3-10 membered heterocyclyl)₂, wherein said 3-10 membered heterocyclyl groups are the same or different and each comprise 1 to 3 heteroatoms selected from the group consisting of N, O, S and P,
j) C₀-C₆alkylene-6-10 membered spirocyclic-heterocyclyl, (e.g. C₁-C₆alkylene-6-10 membered spirocyclic-heterocyclyl) said 6-10 membered spirocyclic-heterocyclyl comprising 1 to 3 heteroatoms independently selected from N, O, S and P, wherein said 6-10 membered spirocyclic-heterocyclyl is substituted with 0 to 3 (e.g. 1 or 2) substituents selected from the group consisting of: i) oxo, ii) C₁-C₃alkyl, iii) C(═O)—C₁-C₃alkyl, iv) 3-6 membered heterocyclyl comprising 1 to 3 heteroatoms selected from the group consisting of N, O, S and P, and v) SO₂—C₁-C₃alkyl.
k) C₀-C₆alkylene-3-10 membered heterocyclyl, said 3-10 membered heterocyclyl comprising 1 to 3 heteroatoms independently selected from the group consisting of N, O, S and P wherein said 3-10 membered heterocyclyl is substituted with 0 to 3 (e.g. 1 or 2) substituents selected from the group consisting of: i) halo (e.g fluoro), ii) cyano, iii) C₁-C₃alkyl, iv) O—C₁-C₃alkyl, v) C(═O)—C₁-C₃alkyl, vi) hydroxyl, vii) a 3-6 membered heterocyclyl comprising 1 to 3 heteroatoms selected from the group consisting of N, O, S and P, viii) oxo, ix) C₁-C₃hydroxyalkyl, x) C₁-C₃alkylene-O—C₁-C₃alkyl, xi) C(═O)—NH₂, xii) C(═O)—N(C₁-C₃alkyl)₂, xiii) C(═O)—NH(C₁-C₃alkyl), xiv) SO₂—C₁-C₃alkyl, xv) C(═O)C₁-C₆hydroxyalkyl and xvi) deuterium, or wherein said 3-10 membered heterocyclyl (e.g. morpholinyl) is perdeuterated,
l) hydroxyl,
m) C₁-C₆hydroxyalkyl,
n) C₀-C₆alkylene-5-6 membered heteroaryl comprising 1 to 3 (e.g. 1 or 2) heteroatoms which are independently selected from the group consisting of N, O and S, and
o) C(═O)-3-10 membered heterocyclyl, said 3-10 membered heterocyclyl comprising 1 to 3 heteroatoms independently selected from the group consisting of N, O, S and P, wherein said 3-10 membered heterocyclyl is substituted with 0 to 3 substituents independently selected from the group consisting of i) O—C₁-C₃alkyl and ii) C₁-C₃alkyl, and
p) C₀-C₆alkylene-N(C₁-C₃alkyl)-SO₂-3-10 membered heterocyclyl, said 3-10 membered heterocyclyl comprising 1 to 3 heteroatoms independently selected from the group consisting of N, O, S and P, wherein said 3-10 membered heterocyclyl is substituted with 0 to 3 substituents which are each independently selected from C₁-C₃alkyl;
or wherein Z is —C(R^{1C})₂, and wherein:
a) the two R^{1C} groups together with the carbon atom to which they are mutually attached form a 4 to 6 membered heterocyclyl comprising 1 to 3 heteroatoms independently selected from N, O and S, wherein the 4 to 6 membered heterocyclyl is optionally substituted with one or two substituents independently selected from the group consisting of: i) C₁-C₃alkyl, ii) oxo, iii) 4 to 5 membered heterocyclyl comprising 1 to 3 heteroatoms independently selected from N, O and S, or
   iv) C(═O)C₁-C₃alkyl,
b) one R^{1C} is hydroxyl and the other R^{1C} is C₁-C₆alkyl or C₁-C₆alkylene-3-10 membered heterocyclyl, said 3-10 membered heterocyclyl comprising 1 to 3 heteroatoms selected from the group consisting of N, O, S and P, or
c) the two R^{1C} groups together form oxo;
or a pharmaceutically acceptable salt thereof.

Embodiment 54. A compound according to any one of Embodiments 1, 1a and Embodiment 53 wherein Z is CHR^{1C}, and wherein R^{1C} is selected from the group consisting of
H,
N(C₁-C₆alkyl)-C(═O)—C₁-C₆alkyl,
N(C₁-C₆alkyl)-C(═O)—C₁-C₆alkylene-O—C₁-C₆alkyl,
3-10 membered heterocyclyl, said 3-10 membered heterocyclyl comprising 1 to 3 heteroatoms independently selected from the group consisting of N, O, S and P wherein said 3-10 membered heterocyclyl is substituted with SO₂—C₁-C₆alkyl,
3-10 membered heterocyclyl, said 3-10 membered heterocyclyl comprising 1 to 3 heteroatoms independently selected from the group consisting of N, O, S and P wherein said 3-10 membered heterocyclyl is substituted with one or two halo (e.g. fluoro) groups,
3-10 membered heterocyclyl, said 3-10 membered heterocyclyl comprising 1 to 3 heteroatoms independently selected from the group consisting of N, O, S and P wherein said 3-10 membered heterocyclyl is substituted with a C(═O)—C₁-C₆hydroxyalkyl group,
3-10 membered heterocyclyl, said 3-10 membered heterocyclyl comprising 1 to 3 heteroatoms independently selected from N, O, S and P and wherein said 3-10 membered heterocyclyl is substituted with a cyano group and a C₁-C₃ alkyl group,
3-10 membered heterocyclyl wherein said 3-10 membered heterocyclyl comprises 1 to 3 heteroatoms independently selected from N, O, S and P wherein said 3-10 membered heterocyclyl is substituted with a C₁-C₃ alkyl groups,
3-10 membered heterocyclyl, said 3-10 membered heterocyclyl comprising 1 to 3 heteroatoms selected from the group consisting of N, O, S and P,
C(═O)-3-10 membered heterocyclyl, said 3-10 membered heterocyclyl comprising 1 to 3 heteroatoms independently selected from the group consisting of N, O, S and P, wherein said 3-10 membered heterocyclyl is substituted with a O—C₁-C₃alkyl group,
C(═O)-3-10 membered heterocyclyl, said 3-10 membered heterocyclyl comprising 1 to 3 heteroatoms independently selected from the group consisting of N, O, S and P, wherein said 3-10 membered heterocyclyl is substituted with a C₁-C₃alkyl group,
C₁-C₆alkylene-3-10 membered heterocyclyl, said 3-10 membered heterocyclyl comprising 1 to 3 heteroatoms independently selected from the group consisting of N, O, S and P, wherein said 3-10 membered heterocyclyl is substituted with a 0-C₁-C₃alkyl group,
C₁-C₆alkylene-3-10 membered heterocyclyl, said 3-10 membered heterocyclyl comprising 1 to 3 heteroatoms independently selected from the group consisting of N, O, S and P, wherein said 3-10 membered heterocyclyl is substituted with a C(═O)—C₁-C₃alkyl groups,
C₁-C₆alkylene-3-10 membered heterocyclyl (e.g. —CH₂-morpholinyl), said 3-10 membered heterocyclyl comprising 1 to 3 heteroatoms independently selected from the group consisting of N and O, wherein said 3-10 membered heterocyclyl is perdeuterated,
N(C₁-C₆alkylene-O—C₁-C₆alkyl)₂,
C₁-C₆alkylene-N(C₁-C₆alkylene-O—C₁-C₆alkyl)(C₁-C₆hydroxyalkyl),
C₁-C₆alkylene-N(C₁-C₆alkyl)-SO₂-3-10 membered heterocyclyl, said 3-10 membered heterocyclyl comprising 1 to 3 heteroatoms independently selected from the group consisting of N, O, and S, wherein said 3-10 membered heterocyclyl is substituted with a C₁-C₃alkyl group,
3-10 membered heterocyclyl wherein said 3-10 membered heterocyclyl comprises 1 to 3 heteroatoms independently selected from the group consisting of N, O, S and P wherein said 3-10 membered heterocyclyl is substituted by one or two O—C₁-C₃alkyl groups,
N(C₁-C₆alkyl)-3-10 membered heterocyclyl, said 3-10 membered heterocyclyl comprising 1 to 3 heteroatoms independently selected from the group consisting of N, O, S and P,
C₁-C₆alkylene-3-10 membered heterocyclyl, said 3-10 membered heterocyclyl comprising 1 to 3 heteroatoms independently selected from the group consisting of N, O, S and P,
3-10 membered heterocyclyl, said 3-10 membered heterocyclyl comprising 1 to 3 heteroatoms selected from the group consisting of N, O, S and P, wherein said 3-10 membered heterocyclyl is substituted with one hydroxyl group and one C₁-C₃alkyl group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one hydroxyl group and/or one C₁-C₃hydroxyalkyl group,
O—C₁-C₆alkylene-3-10 membered heterocyclyl, said 3-10 membered heterocyclyl comprising 1 to 3 heteroatoms selected from the group consisting of N, O, S and P,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with a 3-6 membered heterocyclyl comprising 1 to 3 heteroatoms selected from the group consisting of N, O, S and P,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with a 3-6 membered heterocyclyl comprising 1 to 3 heteroatoms selected from the group consisting of N, O, S and P, and a C₁-C₃alkylene-O—C₁-C₃alkyl group
C₁-C₆alkylene-N(C₁-C₆alkyl)-C₁-C₆alkylene-C(═O)-3-10 membered heterocyclyl, said 3-10 membered heterocyclyl comprising 1 to 3 heteroatoms selected from the group consisting of N, O, S and P,
C₁-C₆alkylene-N(3-10 membered heterocyclyl)₂, wherein said 3-10 membered heterocyclyl groups are the same or different and each comprise 1 to 3 heteroatoms selected from the group consisting of N, O, S and P,
C₁-C₆alkylene-6-10 membered spirocyclic-heterocyclyl, said 6-10 membered spirocyclic-heterocyclyl comprising 1 to 3 heteroatoms independently selected from N, O, S and P,
C₁-C₆alkylene-6-10 membered spirocyclic-heterocyclyl, said 6-10 membered spirocyclic-heterocyclyl comprising 1 to 3 heteroatoms independently selected from N, O, S and P, wherein said 6-10 membered spirocyclic-heterocyclyl is substituted with a C₁-C₃alkyl group,
C₁-C₆alkylene-6-10 membered spirocyclic-heterocyclyl, said 6-10 membered spirocyclic-heterocyclyl comprising 1 to 3 heteroatoms independently selected from N, O, S and P, wherein said 6-10 membered spirocyclic-heterocyclyl is substituted with an oxo group and one or two C₁-C₃alkyl groups,
C₁-C₆alkylene-6-10 membered spirocyclic-heterocyclyl, said 6-10 membered spirocyclic-heterocyclyl comprising 1 to 3 heteroatoms independently selected from N, O, S and P, wherein said 6-10 membered spirocyclic-heterocyclyl is substituted with C(═O)C₁-C₃alkyl,
C₁-C₆alkylene-6-10 membered spirocyclic-heterocyclyl, said 6-10 membered spirocyclic-heterocyclyl comprising 1 to 3 heteroatoms independently selected from N, O, S and P, wherein said 6-10 membered spirocyclic-heterocyclyl is substituted with 3-6 membered heterocyclyl comprising 1 to 3 heteroatoms selected from the group consisting of N, O, S and P,
C₁-C₆alkylene-6-10 membered spirocyclic-heterocyclyl, said 6-10 membered spirocyclic-heterocyclyl comprising 1 to 3 heteroatoms independently selected from N, O, S and P, wherein said 6-10 membered spirocyclic-heterocyclyl is substituted with C(═O)C₁-C₃alkyl,
C₁-C₆alkylene-6-10 membered spirocyclic-heterocyclyl, said 6-10 membered spirocyclic-heterocyclyl comprising 1 to 3 heteroatoms independently selected from N, O, S and P, wherein said 6-10 membered spirocyclic-heterocyclyl is substituted with S(═O)₂—C₁-C₃alkyl,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one oxo group and one C₁-C₃hydroxyalkyl group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one oxo group and one C₁-C₃alkylene-O—C₁-C₃alkyl group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one oxo group and/or one C₁-C₃alkyl group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one C₁-C₃alkyl group and one C₁-C₃hydroxyalkyl group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one C(═O)—NH₂ group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one C(═O)—N(C₁-C₃alkyl)₂ group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one C(═O)—NH(C₁-C₃alkyl) group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one C(═O)—C₁-C₃alkyl group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one SO₂—C₁-C₃alkyl group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one C₁-C₃hydroxyalkylgroup and one 3-6 membered heterocyclyl comprising 1 to 3 heteroatoms selected from the group consisting of N, O, S and P,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one C₁-C₃alkylgroup and one C₁-C₃hydroxyalkylgroup,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is substituted with one hydroxyl group and one halo (e.g. fluoro) group,
C₁-C₆alkylene-3-10 membered heterocyclyl, said 3-10 membered heterocyclyl comprising 1 to 3 heteroatoms selected from the group consisting of N, O, S and P, wherein said 3-10 membered heterocyclyl is deuterated (e.g. fully deuterated),
hydroxyl,
C₁-C₆hydroxyalkyl, and
5-6 membered heteroaryl comprising 1 or 2 heteroatoms which are independently selected from the group consisting of N, O and S,
C₁-C₆alkylene-5-6 membered heteroaryl comprising 1 or 2 heteroatoms which are independently selected from the group consisting of N, O and S;
or wherein Z is C(R^{1C})₂, and wherein the two R^{1C} groups together with the carbon atom to which they are mutually attached form:
   a) a 4 to 6 membered heterocyclyl comprising 1 to 3 heteroatoms independently selected from N, O and S, wherein the 4 to 6 membered heterocyclyl is substituted with one C₁-C₃alkyl group,
   b) a 4 to 6 membered heterocyclyl comprising 1 to 3 heteroatoms independently selected from N, O and S,
   c) a 4 to 6 membered heterocyclyl comprising 1 to 3 heteroatoms independently selected from N, O and S wherein the 4 to 6 membered heterocyclyl is substituted with one C₁-C₃alkyl group and one oxo group,
   d) a 4 to 6 membered heterocyclyl comprising 1 to 3 heteroatoms independently selected from N, O and S wherein the 4 to 6 membered heterocyclyl is substituted with a 4 to 5 membered heterocyclyl comprising 1 or 2 heteroatoms independently selected from N, O and S, or
   e) a 4 to 6 membered heterocyclyl comprising 1 to 3 heteroatoms independently selected from N, O and S, wherein the 4 to 6 membered heterocyclyl is substituted with one C(═O)C₁-C₃alkyl group;
or wherein Z is CR^{1C}₂, and wherein one R^{1C} is hydroxyl and the other R^{1C} is C₁-C₆alkyl or C₁-C₆alkylene-3-10 membered heterocyclyl said 3-10 membered heterocyclyl comprising 1 to 3 heteroatoms selected from the group consisting of N, O, S and P,
or wherein Z is C(R^{1C})₂, and the two R^{1C} groups together form oxo,
or a pharmaceutically acceptable salt thereof.

Embodiment 55. A compound according to any one of Embodiments 1, 1a 53 and 54 or wherein Z is CHR^{1C} and R^{1C} is selected from the group consisting of H, N(C₁alkyl)-C(═O)—C₁alkyl, N(C₁alkyl)-C(═O)—C₁alkylene-O—C₁alkyl,

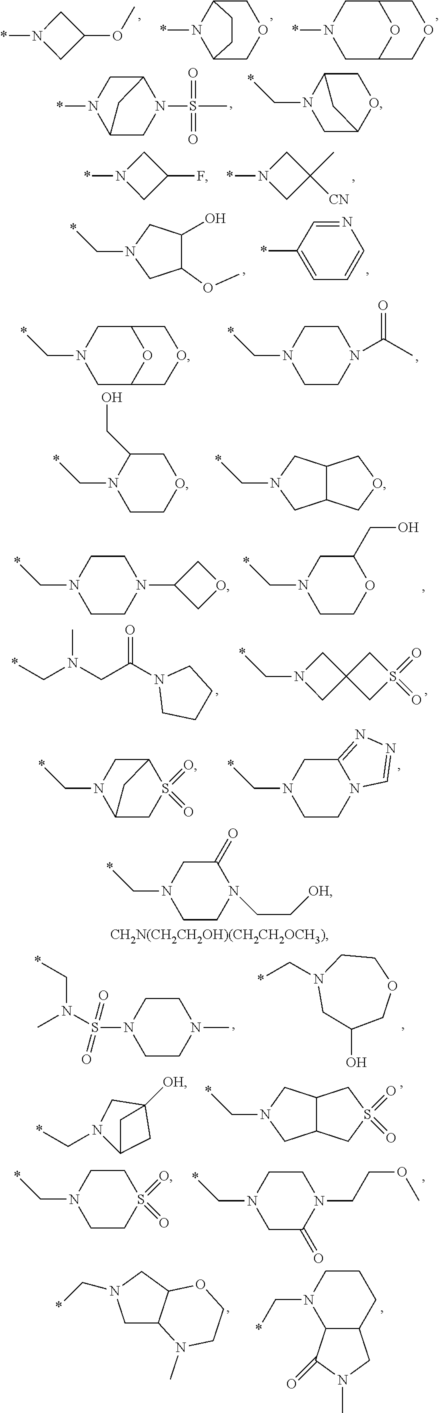

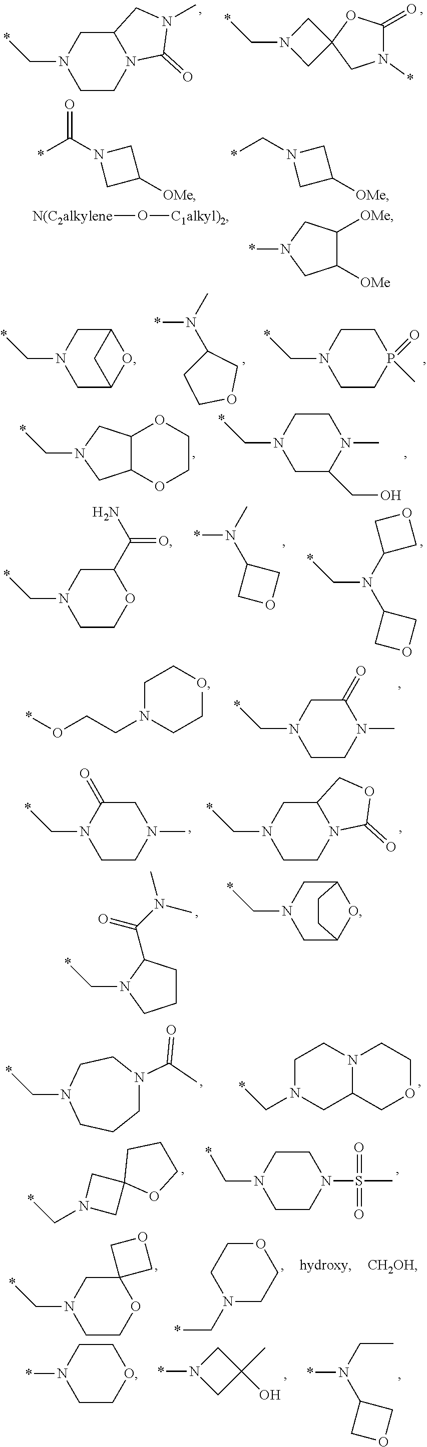

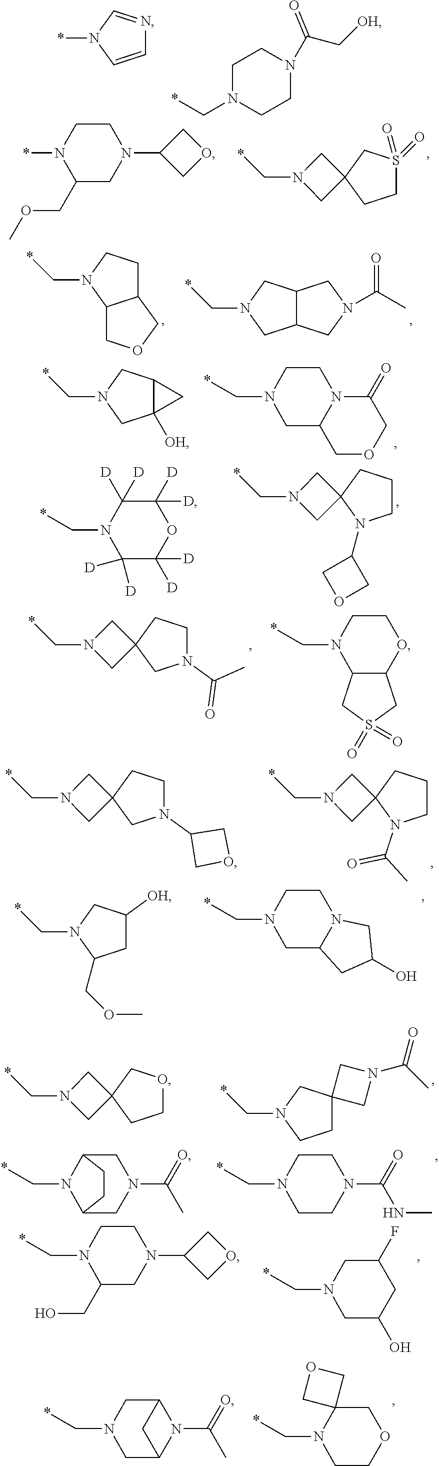

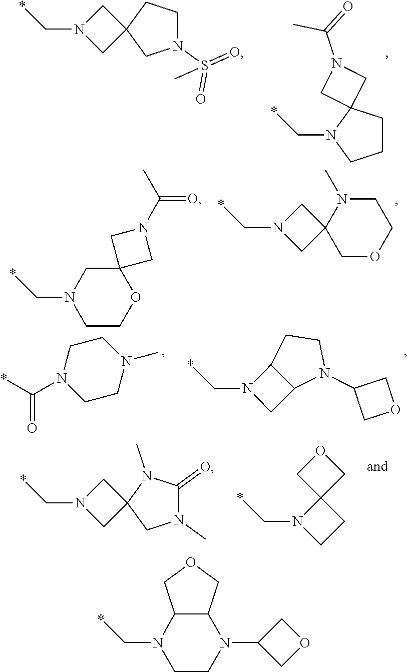

or wherein Z is C(R^{1C})₂, and wherein the two R^{1C} groups together form:

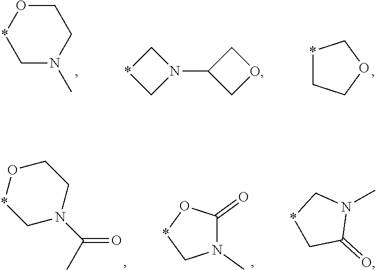

or oxo,
or wherein Z is C(R^{1C})₂, and wherein one R^{1C} is hydroxyl and the other R^{1C} is C₁alkyl or

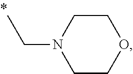

wherein * indicates the point of attachment to the remainder of the molecule, or a pharmaceutically acceptable salt thereof.

Embodiment 56. A compound selected from any of Examples 1-320, or a pharmaceutically acceptable salt thereof.

(It should be understood that Embodiment 56 includes all of “a”, “b”, “c” and “d” exemplary compounds)

Embodiment 56a. A compound of formula (I) according to Embodiment 1, or a pharmaceutically acceptable salt thereof, wherein:
Ring A is a 7 to 10 membered spirocyclic-heterocyclylene comprising 1 N heteroatom, wherein said 7 to 10 membered spirocyclic-heterocyclylene is unsubstituted;
G is CR¹²
R^{Z} is

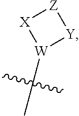

wherein
W is N;
   i) X is **—CR²₂—(CR³₂)ₙ—*, Y is **—CR⁴₂—(CR⁵₂)ₘ—*, and Z is selected from the group consisting of NR^{1N} and C(R^{1C})₂, where the * of X indicates the point of attachment to Z and the ** of X indicates the point of attachment to W, and where the * of Y indicates point of attachment to Z and the ** of Y indicates point of attachment to W, n is 0, 1 or 2 and m is 0, 1 or 2;
R^{1N} is selected from the group consisting of H and -L^{N}-R^{2N}, preferably wherein R^{1N} is -L^{N}-R^{2N};
R^{1C}, where present, is at each occurrence independently selected from the group consisting of H and -L^{C}-R^{2C};
L^{N} is selected from the group consisting of a bond, and C₁-C₆alkylene,
R^{2N} is a 3-10 membered heterocyclyl comprising 1 to 3 heteroatoms independently selected from N, O and S substituted with 0 to 3 (e.g. 0 to 2) substituents R^{x},
L^{C} is selected from the group consisting of a bond, and C₁-C₆alkylene (e.g. L^{C} is C₁alkylene); wherein R^{2C} is a 3-10 membered heterocyclyl comprising 1 to 3 heteroatoms independently selected from N, O and S substituted by 0 to 2 substituents R^{x},
   R², R³, R⁴ and R⁵ are each independently selected from the group consisting of H, C₁-C₆alkyl (e.g., Me, Et, iPr), halo, C₁-C₆alkylene-O—C₁-C₆alkyl; and/or
   i) an R² group and an R⁴ group in combination form a bridging group;
   ii) an R² group and an R⁵ group in combination form a bridging group;
   iii) an R³ group and an R⁴ group in combination form a bridging group; or
   iv) an R³ group and an R⁵ group in combination form a bridging group;
wherein the bridging group forms a C₄-C₆cycloalkyl, or a 4 to 6 membered heterocyclyl comprising 1 to 3 heteroatoms independently selected from the group consisting of N, O, S and P (preferably selected from the group consisting of N, O and S), wherein the C₄-C₆cycloalkyl or 4 to 6 membered heterocyclyl are each substituted with 0 to 3 substituents R^{x};
   each R^{x} is independently selected from a) C₁-C₃alkyl (e.g., Me, Et, iPr), b) halo (preferably fluoro), c) C(═O)—C₁-C₃alkyl, h) oxo, o) C₁-C₃alkylene-O—C₁-C₃alkyl, q) 3 to 6 membered heterocyclyl comprising 1 to 3 heteroatoms (preferably 1 or 2 heteroatoms) independently selected from the group consisting of N, O and S (preferably selected from the group consisting of N and O) substituted by 0 to 2 (preferably 0) substituents selected from the group consisting of CH₃, OH, OMe, F and CN,
   R⁶ is CH═CH₂;
   R⁸ is H, halo (preferably chloro) or C(R^{8a})₃, wherein each R^{8a} is independently selected from the group consisting of H, C₁-C₃alkyl, and halo (preferably fluoro), preferably wherein each R^{8a} is H or wherein each R^{8a} is D, and
   R⁹ is H, halo (preferably fluoro or chloro), or C(R^{9a})₃, wherein each R^{9a} is independently selected from the group consisting of H, C₁-C₃alkyl, and halo (preferably fluoro), preferably wherein each R^{9a} is H or wherein each R^{9a} is D,
   R¹⁰ and R¹¹ are joined together to form, in combination with the 6 membered aryl to which they are mutually attached, a 9 membered fused bicyclic heteroaryl group containing 1 to 3 (preferably 2) heteroatoms independently selected from the group consisting of N, O, and S (preferably wherein the heteroatom(s) are independently selected from the group consisting of N and O, more preferably wherein each heteroatom is N), wherein said fused bicyclic heteroaryl group is substituted with 0 to 3 substituents independently selected from the group consisting of C₁-C₆alkyl (preferably methyl), and NH₂;
   R¹² is H, halo (preferably fluoro) or methyl, preferably R¹² is H;
   R^{a} is H or C(R¹³)₃, and
   each R¹³ is independently selected from the group consisting of H and deuterium.

Embodiment 57. A compound selected from the group consisting of:

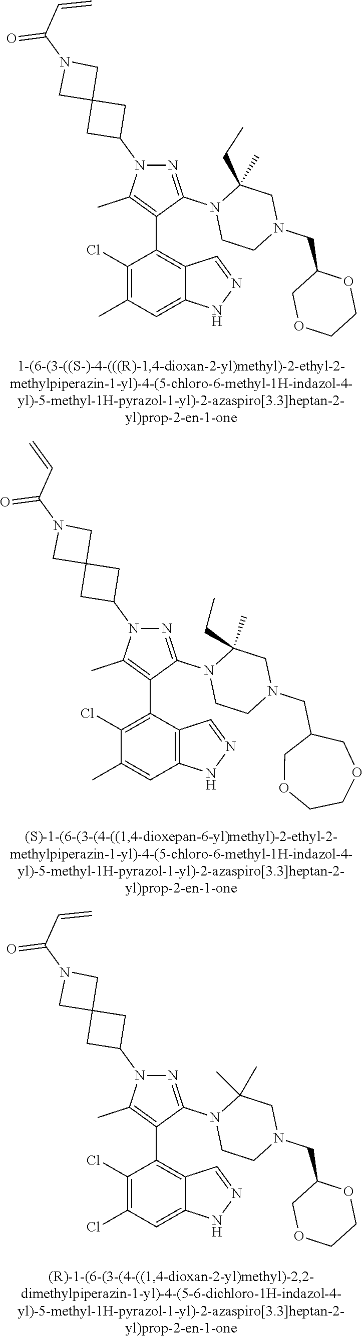

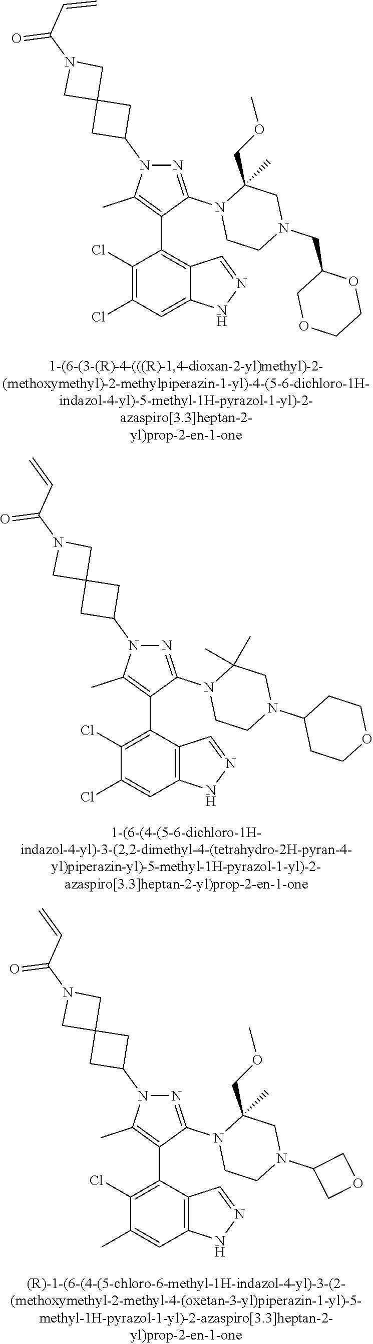

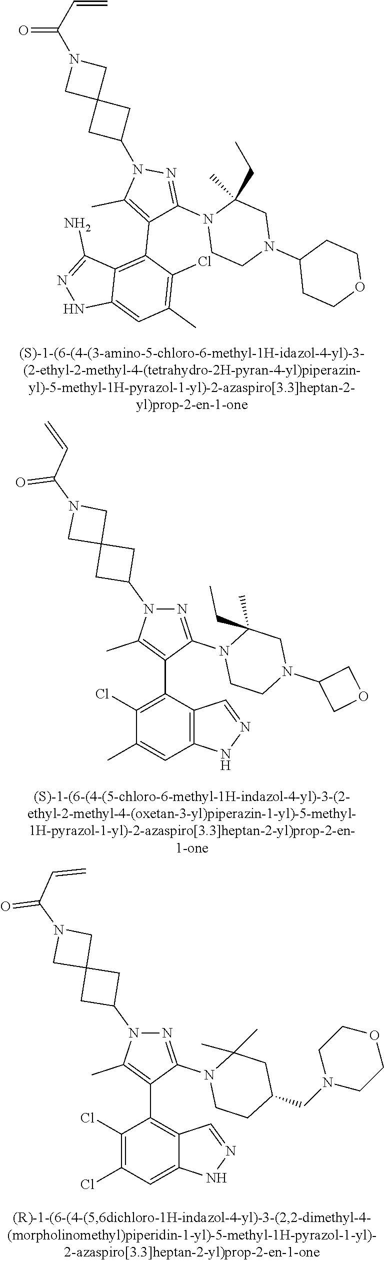

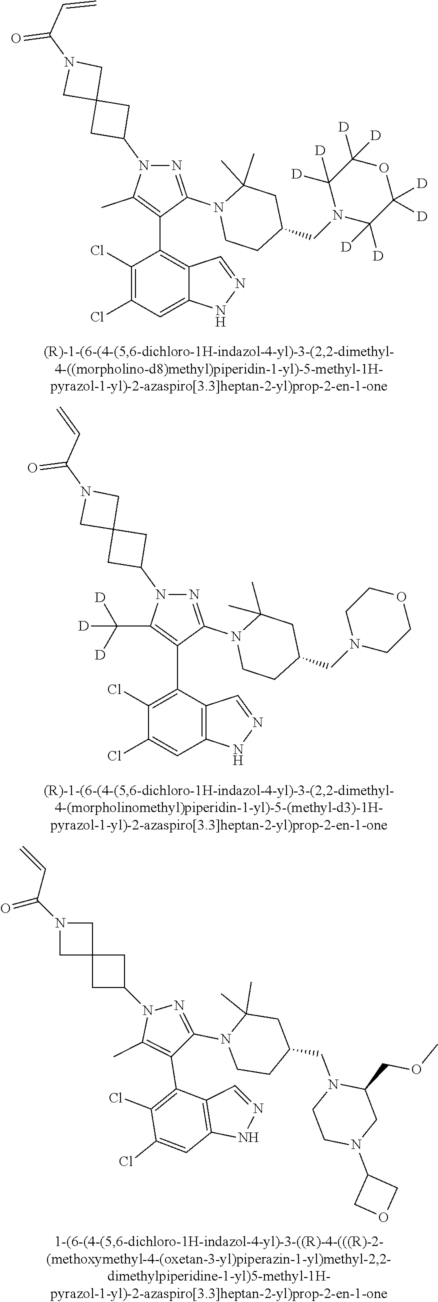 [SMILES: [2H]C1([2H])OC([2H])([2H])C([2H])([2H])N(C[C@@H]2CCN(c3nn(C4CC5(C4)CN(C(=O)C=C)C5)c(C)c3-c3c(Cl)c(Cl)cc4[nH]ncc34)C(C)(C)C2)C1([2H])[2H] ; [2H]C([2H])([2H])c1c(-c2c(Cl)c(Cl)cc3[nH]ncc23)c(N2CC[C@@H](CN3CCOCC3)CC2(C)C)nn1C1CC2(C1)CN(C(=O)C=C)C2 ; C=CC(=O)N1CC2(CC(n3nc(N4CC[C@@H](CN5CCN(C6COC6)C[C@@H]5COC)CC4(C)C)c(-c4c(Cl)c(Cl)cc5[nH]ncc45)c3C)C2)C1]

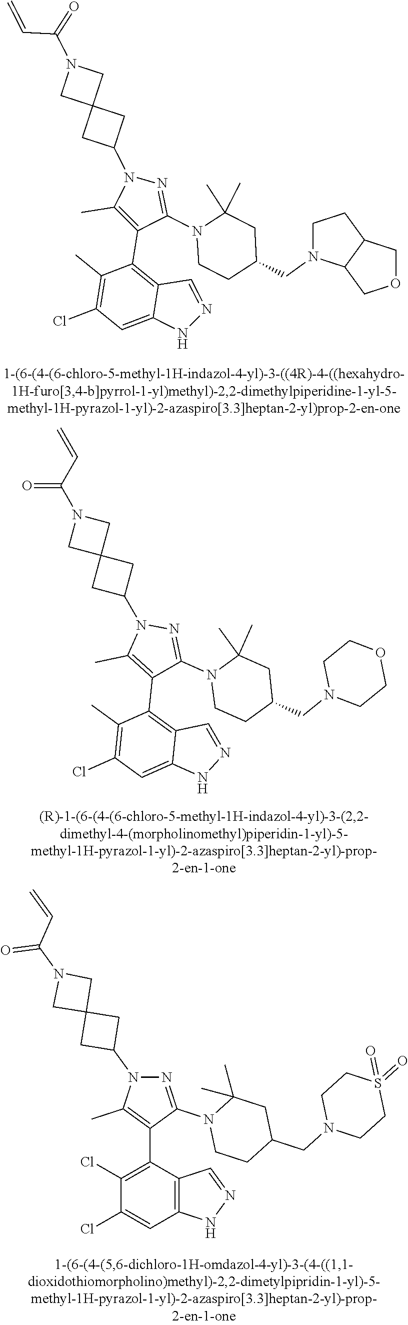 [SMILES: C=CC(=O)N1CC2(CC(n3nc(N4CC[C@@H](CN5CCC6COCC65)CC4(C)C)c(-c4c(C)c(Cl)cc5[nH]ncc45)c3C)C2)C1 ; C=CC(=O)N1CC2(CC(n3nc(N4CC[C@@H](CN5CCOCC5)CC4(C)C)c(-c4c(C)c(Cl)cc5[nH]ncc45)c3C)C2)C1 ; C=CC(=O)N1CC2(CC(n3nc(N4CCC(CN5CCS(=O)(=O)CC5)CC4(C)C)c(-c4c(Cl)c(Cl)cc5[nH]ncc45)c3C)C2)C1]

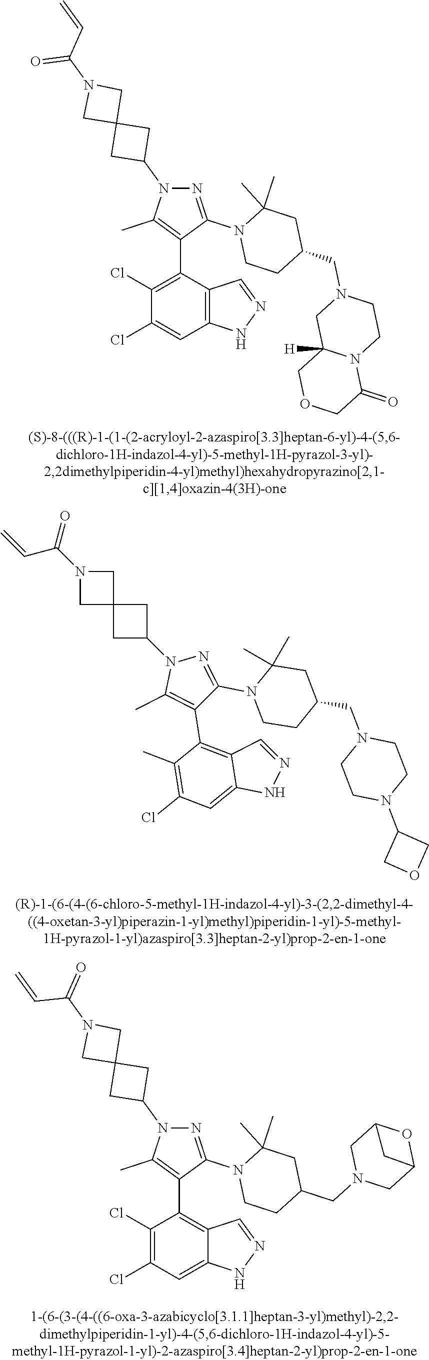 [SMILES: C=CC(=O)N1CC2(CC(n3nc(N4CC[C@@H](CN5CCN6C(=O)COC[C@@H]6C5)CC4(C)C)c(-c4c(Cl)c(Cl)cc5[nH]ncc45)c3C)C2)C1 ; C=CC(=O)N1CC2(CC(n3nc(N4CC[C@@H](CN5CCN(C6COC6)CC5)CC4(C)C)c(-c4c(C)c(Cl)cc5[nH]ncc45)c3C)C2)C1 ; C=CC(=O)N1CC2(CC(n3nc(N4CCC(CN5CC6CC(C5)O6)CC4(C)C)c(-c4c(Cl)c(Cl)cc5[nH]ncc45)c3C)C2)C1]

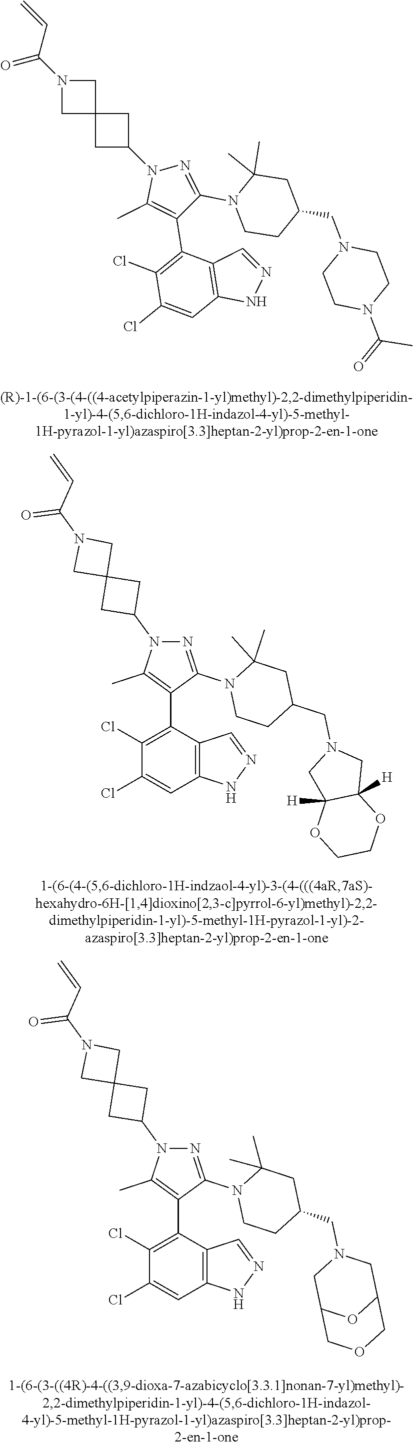 [SMILES: C=CC(=O)N1CC2(CC(n3nc(N4CC[C@@H](CN5CCN(C(C)=O)CC5)CC4(C)C)c(-c4c(Cl)c(Cl)cc5[nH]ncc45)c3C)C2)C1 ; C=CC(=O)N1CC2(CC(n3nc(N4CCC(CN5C[C@@H]6OCCO[C@@H]6C5)CC4(C)C)c(-c4c(Cl)c(Cl)cc5[nH]ncc45)c3C)C2)C1 ; C=CC(=O)N1CC2(CC(n3nc(N4CC[C@@H](CN5CC6COCC(C5)O6)CC4(C)C)c(-c4c(Cl)c(Cl)cc5[nH]ncc45)c3C)C2)C1]

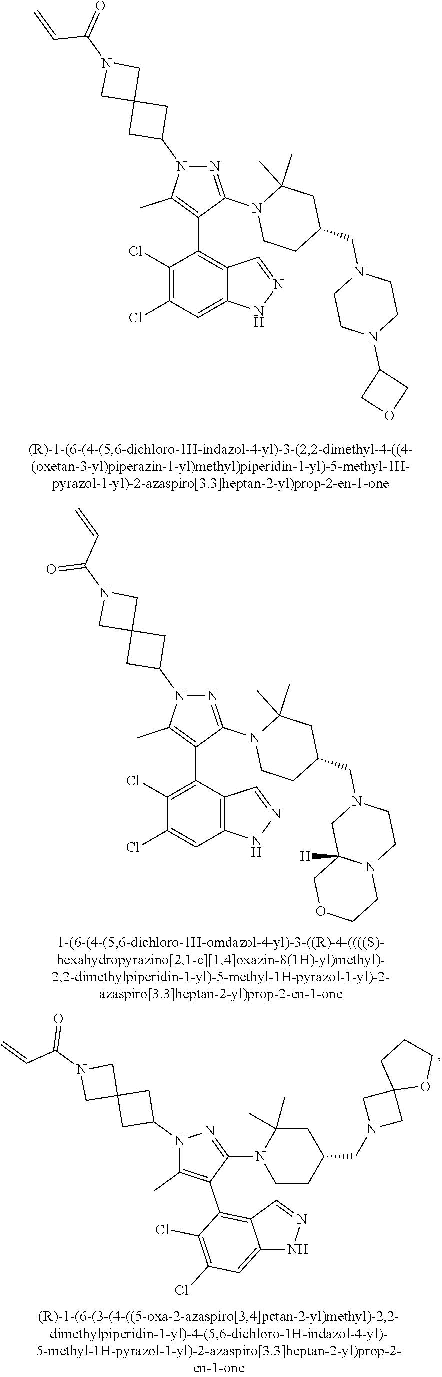 [SMILES: C=CC(=O)N1CC2(CC(n3nc(N4CC[C@@H](CN5CCN(C6COC6)CC5)CC4(C)C)c(-c4c(Cl)c(Cl)cc5[nH]ncc45)c3C)C2)C1 ; C=CC(=O)N1CC2(CC(n3nc(N4CC[C@@H](CN5CCN6CCOC[C@@H]6C5)CC4(C)C)c(-c4c(Cl)c(Cl)cc5[nH]ncc45)c3C)C2)C1 ; C=CC(=O)N1CC2(CC(n3nc(N4CC[C@@H](CN5CC6(CCCO6)C5)CC4(C)C)c(-c4c(Cl)c(Cl)cc5[nH]ncc45)c3C)C2)C1]

or a pharmaceutically acceptable salt thereof.

Embodiment 58. A pharmaceutical composition comprising a compound according to any one of the preceding Embodiments, or a pharmaceutically acceptable salt thereof and at least one pharmaceutically acceptable carrier.

Embodiment 59. A compound or a pharmaceutically acceptable salt thereof according to any one of Embodiments 1 to 57, or a pharmaceutical composition according to Embodiment 58 for use as a medicament.

Embodiment 60. A compound or a pharmaceutically acceptable salt thereof according to any one of Embodiments 1 to 57, or a pharmaceutical composition according to Embodiment 58 for use in the treatment of cancer.

Embodiment 61. A method of treating cancer, the method comprising administering a therapeutically effective amount of a compound or a pharmaceutically acceptable salt thereof according to any one of Embodiments 1 to 57, or a pharmaceutical composition according to Embodiment 58 to a patient in need thereof.

Embodiment 62. Use of a compound or pharmaceutically acceptable salt thereof according to any one of Embodiments 1 to 57, or a pharmaceutical composition according to Embodiment 58 in a method of treating cancer.

Embodiment 63. Use of a compound or pharmaceutically acceptable salt according to any one of Embodiments 1 to 57, or a pharmaceutical composition according to Embodiment 58 in the manufacture of a medicament for treating cancer.

Embodiment 64. The compound for use or composition for use according to Embodiment 60, the method according to Embodiment 61 or use according to Embodiment 62 or Embodiment 63 wherein the cancer is selected from the group consisting of lung cancer (including lung adenocarcinoma and non-small cell lung cancer), colorectal cancer (including colorectal adenocarcinoma), pancreatic cancer (including pancreatic adenocarcinoma), uterine cancer (including uterine endometrial cancer) and rectal cancer (including rectal adenocarcinoma).

Embodiment 65. The compound for use, composition for use, method or use according to Embodiment 64 wherein the cancer is mediated by a KRAS, NRAS or GRAS G12C mutation.

Embodiment 66. A combination comprising a compound or pharmaceutically acceptable salt thereof according to any one of Embodiments 1 to 57, or a pharmaceutical composition according to Embodiment 58 and one or more therapeutically active agents.

Embodiment 67. A method of inhibiting the G12C mutant KRAS, HRAS or NRAS protein in a subject in need thereof, wherein the method comprises administering to the subject a therapeutically effective amount of a compound or pharmaceutically acceptable salt thereof according to any one of Embodiments 1 to 57, or a pharmaceutical composition according to Embodiment 58.

In an embodiment of the invention, the compound of formula (I) or pharmaceutically acceptable salt thereof is a compound of formula (Ia),

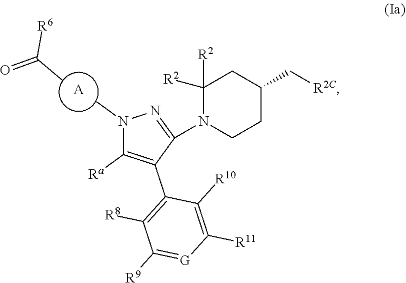

or a pharmaceutically acceptable salt thereof. In this embodiment, G, Ring A, R^{a}, R², R^{2C}, R⁶, R⁸, R⁹, R¹⁰ and R¹¹ are as defined in any one of numbered embodiments 1 to 49 and 53 to 55 above.

In an alternative embodiment of the invention, the compound of formula (I) or pharmaceutically acceptable salt thereof is a compound of formula (Ib),

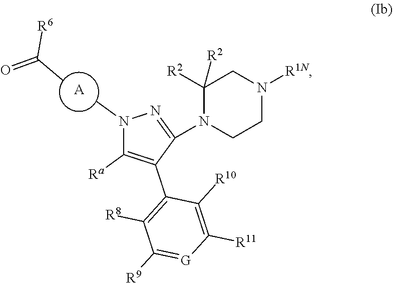

or a pharmaceutically acceptable salt thereof. In this embodiment, G, Ring A, R^{a}, R², R^{1N}, R⁶, R⁸, R⁹, R¹⁰ and R¹¹ are as defined in any one of numbered embodiments 1 to 52 above.

Unless specified otherwise, the term “compounds of the present invention” or “compound of the present invention” refers to compounds of formula (I), subformulae thereof and exemplified compounds, as well as pharmaceutically acceptable salts thereof, including all stereoisomers (including diastereoisomers, enantiomers and atropisomers thereof), tautomers and isotopically labeled compounds (including deuterium substitutions), as well as inherently formed moieties. Where one isomer (enantiomer, diastereomer, atropisomer, or geometric isomer) has higher intrinsic activity as an inhibitor of RAS G12C mutant protein than its opposite isomer, the more active isomer is typically preferred.

The presence of diastereoisomers can be identified by a person of skill in the art with tools such as NMR. Separation of diastereoisomers can be carried out by a person of skill in the art using chromatographic methods, with tools such as HPLC (High Performance Liquid Chromatography), Thin Layer Chromatography, SFC (Supercritical Fluid Chromatography), GC (Gas Chromatography), or recrystallization techniques. Separation of enantiomers can be carried out by a person of skill in the art with tools such as chiral HPLC, chiral SFC, chiral GC.

Compounds of the present invention, in particular, ortho-substituted biaryl compounds may exhibit conformational, rotational isomerism, herein referred to as atropisomerism (Eliel, E. and Wilen, S. (1994) Stereochemistry of Organic Compounds, John Wiley & Sons, Inc., pp. 1142-55). In other words, depending upon the substituents on the bi-aryl ring moiety, such biaryl compounds of the present invention exhibit atropisomerism.

Thus, the compounds of formula (I), and subformulae thereof and their isomeric mixtures (including diastereomeric mixtures, enantiomeric mixtures, atropisomeric mixtures and racemic mixtures), also form part of the invention. Likewise, “diastereomerically enriched”, “atropisomerically enriched” and/or “entantiomerically enriched” mixtures of the compounds of formula (I) [including, for example substantially pure diastereoisomers and/or atropisomers of formula (I)], and subformulae thereof also form part of the invention.

The following definitions also apply unless otherwise provided or apparent from context:

As used herein, the term “halogen” “halo”, “hal”, etc. refers to fluorine, bromine, chlorine or iodine. Halogen-substituted groups and moieties, such as alkyl substituted by halogen (haloalkyl) can be mono-, poly- or per-halogenated. Chloro and fluoro are preferred halo substituents on alkyl or cycloalkyl groups, with fluoro being most preferred, unless otherwise specified. Fluoro, chloro and bromo, are often preferred on aryl or heteroaryl groups unless otherwise specified.

The term “heteroatom” refers to an atom which is neither carbon nor hydrogen. Heteroatoms include (but are not limited to) N, O, S, F, Cl, Br, P, I, Se and Si. Most preferably, heteroatoms present in the compounds of the present invention are selected from the group consisting of N, O, S, F and Cl.

Ring heteroatoms, unless indicated otherwise are preferably selected from the group consisting of N, O, S and P. More preferably, ring heteroatoms in the present invention are selected from N, O and S. Most preferably, ring heteroatoms in the present invention are selected from N and O. Where “heteroatom” or “heteroatoms” is mentioned for a ring, this refers to ring heteroatoms. Where N is a ring heteroatom the N is typically (valency permitting) bonded to hydrogen. Said hydrogen may, however, be replaced by another group (which may be described as a substituent) if the ring is described as substituted. Alternatively, the substituent can be attached to a carbon ring atom. Where S is a ring heteroatom, the S may be in the form of S, SO, or SO₂. Where P is a ring heteroatom, the P is preferably in the form of P(═O)C₁-C₃alkyl, most preferably P(═O)C₁alkyl.

As used herein, the term “heterocyclyl”, “heterocycle”, “heterocyclic” etc. refers to a heterocyclic radical that is saturated or partially unsaturated but not aromatic, and can be a monocyclic or a polycyclic ring, including a fused or bridged bicyclic ring system (e.g.

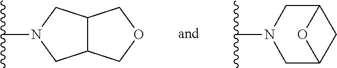 [SMILES: *N1CC2COCC2C1 ; *N1CC2CC(C1)O2]

respectively). A heterocycle or heterocyclyl contains at least one non-carbon atom as a ring member, typically N, O, S or P unless otherwise specified, more typically N, O or S, and yet more typically N or O. Unless otherwise specified, a heterocyclyl group has 3 to 10, preferably 3 to 9, e.g. 4 to 7 ring atoms; wherein one or more, preferably one to four, especially one, two or three ring atoms are heteroatoms independently selected from P, O, S and N (e.g. O, S and N), the remaining ring atoms therefore being carbon.

An unsaturated heterocyclyl can have one or two double bonds, but is not aromatic. Preferably, unless described as unsaturated, the heterocyclyl groups in the compounds of the invention are saturated single rings. Preferably, a heterocyclyl group has one or two heteroatoms as ring atoms, and preferably the heteroatoms are not directly connected to each other. Examples of heterocycles include tetrahydrofuran (THF), dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like.

The term “5-7 membered unsaturated heterocyclyl” refers to a ring radical containing 5 to 7 ring atoms comprising (unless stated otherwise) 1, 2, or 3, heteroatoms individually selected from nitrogen, oxygen and sulfur, and containing one or more C—C double bonds, preferably one C—C double bond. Where the heterocycle contains S or N as heteroatoms, the S may be present as SO or SO₂ groups and the N may be present as the N-oxide, where valency allows. The term includes a 5-, 6- or 7-membered non-aromatic monocyclic ring radical containing one or more C—C double bonds, preferably one C—C double bond, and 1, 2, or 3, heteroatoms individually selected from nitrogen, oxygen and sulfur, preferably one oxygen. Examples of 5-7 membered unsaturated heterocyclyls include, but are not limited to, 6-membered non-aromatic monocyclic radicals containing one oxygen and a C—C double bond such as 3,4-dihydro-2-H-pyranyl, 5,6-dihydro-2H-pyranyl and 2H-pyranyl.

As used herein, the term “carbocyclyl”, “carbocycle”, “carbocyclic” etc. refers to a cyclic radical containing only carbon and hydrogen atoms that is saturated or partially unsaturated but not aromatic, and can be a monocyclic or a polycyclic ring, including a fused or bridged bicyclic ring system. Each ring atom is a carbon atom.

The term “carbocyclyl”, “carbocycle”, “carbocyclic” etc. therefore encompasses cycloalkyls and cycloalkenes, As used herein, the term “cycloalkyl” refers to a saturated carbocyclic ring radical. C₃-C₇ cycloalkyl is any such ring radical containing 3 to 7 carbon atoms such as cyclopropyl, cyclobutyl, cyclopentyl, cyclohexyl and cycloheptyl. As used herein, the term “cycloalkylene” refers to a non-aromatic carbocyclic ring radical containing one double bond. The term “monocyclic cycloalkylene” refers to a non-aromatic monocyclic carbocyclic ring radical containing one double bond. The term includes, but is not limited to “C₅-C₇-cycloalkylene” which is a non-aromatic carbocyclic ring radical containing 5 to 7 carbon atoms and one C—C double bond. Examples of suitable cycloalkylene groups are non-aromatic carbocyclic ring containing 5 to 7 carbon atoms and one or more C—C double bonds such as cyclopentenyl, cyclohexenyl (e.g., cyclohex-1-en-1-yl, cyclohex-2-en-1-yl, cyclohex-3-en-1-yl).

As used herein, the term “spirocyclic-heterocyclyl”, “spirocyclic-heterocyclylene” “spirocyclic-heterocycle”, “spirocyclic-heterocyclic” etc. refers to ring system comprising a first carbocylic or heterocyclic ring comprising from 3 to 8 ring atoms wherein two of the substituents on a carbon ring atom in said first carbocyclic or heterocyclic ring join together to form a second carbocyclic or heterocyclic ring comprising from 3 to 8 ring atoms, with the proviso that at least one of the first and second rings is a heterocyclic ring comprising one or more heteroatoms selected from the group consisting of N, O, S and P (e.g. one or more heteroatoms selected from the group consisting of N, O and S, e.g. one or more heteroatoms selected from N and O). The term 6 to 10 membered spirocyclic-heterocyclyl, as used herein means that the total number of ring atoms in the first carbocyclic or heterocyclic ring and the second carbocyclic or heterocyclic ring is from 6 to 10. For instance, the spirocyclic-heterocyclyl

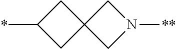

is a 7 membered spirocyclic heterocyclylene, as there are 7 ring atoms present. As will be appreciated by the skilled person, a “spirocyclic-heterocyclyl” is a mono-radical, whereas a “spirocyclic-heterocyclylene” is a di-radical (analogous to alkyl and alkylene).

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. Examples of C₁-C₆-alkyl include, but are not limited to, methyl, ethyl, n-propyl, n-hexane, 1-methylethyl (iso-propyl) and n-butyl. A preferred example is methyl. Preferably each “C₁-C₆alkyl” is a “C₁-C₄alkyl”—i.e. which includes from one to four carbon atoms.

As used herein, the term “aryl” refers to an aromatic hydrocarbon group having 6-14 carbon atoms in the ring portion. Typically, aryl is monocyclic, bicyclic or tricyclic (preferably monocyclic) aryl having 6-14 carbon atoms, often 6-10 carbon atoms, e.g., phenyl or naphthyl. Furthermore, the term “aryl” as used herein, refers to an aromatic substituent which can be a single aromatic ring, or multiple aromatic rings that are fused together. Non-limiting examples include phenyl, naphthyl and 1,2,3,4-tetrahydronaphthyl, provided the tetrahydronaphthyl is connected to the formula being described through a carbon of the aromatic ring of the tetrahydronaphthyl group. Phenyl is generally preferred unless stated otherwise. The term “phenyl” refers to a radical of the formula —C₆H₅. In substituted phenyl, one or more or the hydrogen atoms in —C₆H₅ are replaced with a substituent or with substituents, especially any one described herein.

The term “heteroaryl” is a 5-14 membered, typically 5-10 membered, most typically 5-6 membered monocyclic or bicyclic (preferably monocyclic) aromatic ring radical which, unless otherwise stated, comprises 1, 2, 3 or 4 heteroatoms individually selected from nitrogen, oxygen and sulfur in the ring radical. Typically, the heteroaryl is a 5-10 membered ring system, e.g., a 5-6 membered monocyclic. Typical heteroaryl groups include 2- or 3-thienyl, 2- or 3-furyl, 2- or 3-pyrrolyl, 2-, 4-, or 5-imidazolyl, 1-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-(1,2,4-triazolyl), 4- or 5-(1,2,3-triazolyl), 1- or 2- or 3-tetrazolyl, 2-, 3-, or 4-pyridyl, 3- or 4-pyridazinyl, 2-pyrazinyl, and 2-, 4-, or 5-pyrimidinyl.

A substituted heteroaryl is a heteroaryl group having one or more substituents, typically 1, 2 or 3 substituents, on the heteroaryl ring replacing a hydrogen atom that would be on the unsubstituted heteroaryl.

The term “5-6 membered heteroaryl” is an aromatic monocyclic ring radical which, unless otherwise stated, comprises 1, 2, 3 or 4 heteroatoms individually selected from nitrogen, oxygen and sulfur. The term includes a 5- or 6-membered aromatic ring radical containing 1, 2 or 3 heteroatoms selected from N, O and S as ring members, preferably 1 or 2 heteroatoms independently selected from N and O as ring members. The term includes 6-membered rings in which an aromatic tautomer exists, as for example in the case for the 1H-pyridin-2-one system. Examples of suitable 5-6 membered heteroaryl groups include, but are not limited to, 2- or 3-thienyl, 2- or 3-furyl, 2- or 3-pyrrolyl, 2-, 4-, or 5-imidazolyl, 1-, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-(1,2,4-triazolyl), 4- or 5-(1,2,3-triazolyl), 1- or 2- or 3-tetrazolyl, 2-, 3-, or 4-pyridyl, 3- or 4-pyridazinyl, 2-pyrazinyl, and 2-, 4-, or 5-pyrimidinyl.

As used herein, the term “C₁-C₆hydroxyalkyl” refers to a C₁-C₆alkyl radical as defined herein, wherein one of the hydrogen atoms of the C₁-C₆alkyl radical is replaced by OH. Examples of C₁-C₆hydroxyalkyl include, but are not limited to, hydroxy-methyl, 2-hydroxy-ethyl, 2-hydroxy-propyl, 3-hydroxy-propyl and 5-hydroxy-pentyl.

As used herein, the term “SO₂—C₁-C₆-alkyl” refers to a C₁-C₆alkyl radical as defined herein, which is attached to the rest of the molecule via an S(═O)₂ linker.

As used herein, the term “SO₂-3-10 membered heterocyclyl” refers to a 3-10 membered heterocyclyl as defined herein, which is attached to the rest of the molecule via an S(═O)₂ linker. As used herein, the term “O—C₁-C₆alkyl” refers to C₁-C₆alkyl radical as defined herein, which is attached to the rest of the molecule via an O linker.

As used herein, the term “C₁-C₆alkylene-O—C₁-C₆alkyl” refers to a C₁-C₆alkyl radical as defined herein, wherein one of the hydrogen atoms of said C₁-C₆ alkyl radical has been replaced with —O—C₁-C₆alkylene (attached to the C₁-C₆alkyl radical through the oxygen). An example of “C₁-C₆alkylene-O—C₁-C₆alkyl” is —CH₂—O—CH₃.

As used herein, the term “O—C₁-C₆alkylene-O—C₁-C₆alkyl” refers to a C₁-C₆alkyl radical as defined herein, wherein one of the hydrogen atoms of said C₁-C₆ alkyl radical has been replaced with 0-C₁-C₆alkylene-O.

As used herein the term “C₁-C₆haloalkyl” refers to a C₁-C₆alkyl radical as defined herein, wherein one or more of the hydrogen atoms of said C₁-C₆ alkyl has been replaced with a halogen atom. Preferably said one or more halogen atom(s) are each fluorine atom(s), in which case the “C₁-C₆haloalkyl” is a “C₁-C₆fluoroalkyl”.

As used herein, the term “O—C₁-C₆haloalkyl” refers to C₁-C₆haloalkyl radical as defined herein, which is attached to the rest of the molecule via an O linker.

As used herein the term “C₁-C₆hydroalkyl” refers to a C₁-C₆alkyl radical as defined herein, wherein one or more of the hydrogen atoms of said C₁-C₆ alkyl has been replaced with hydroxyl. As will be appreciated by one of normal skill in the art, only one hydrogen atom on any given carbon atom in said C₁-C₆ alkyl radical can be replaced by hydroxyl.

As used herein, the term “amino” refers to an —NH₂ group.

As used herein, the term “C₁-C₆alkylamino” refers to a C₁-C₆alkyl radical as defined herein, wherein one or more of the hydrogen atoms of said C₁-C₆ alkyl radical has been replaced with an amino group. As will be appreciated by one of normal skill in the art, only one hydrogen atom on any given carbon atom in said C₁-C₆ alkyl radical can be replaced by an amino group.

As used herein the term “C₁-C₆cyanoalkyl” refers to a C₁-C₆alkyl radical as defined herein, wherein all three hydrogen atoms on any given terminal carbon atom of said C₁-C₆ alkyl has been replaced with ≡N.

As used herein the term “C₁-C₆alkylene-C₃-C₈cycloalkyl” refers to a C₃-C₈cycloalkyl as defined herein, wherein one of the hydrogen atoms of said C₃-C₈cycloalkyl has been replaced with a C₁-C₆alkylene as defined herein.

As used herein the term “C₁-C₆alkylene-3-10 membered heterocyclyl comprising 1 to 3 heteroatoms independently selected from N, O, S and P” refers to a 3-10 membered heterocyclyl comprising 1 to 3 heteroatoms independently selected from N, O, S and P as defined herein, wherein one of the hydrogen atoms of said 3-10 membered heterocyclyl comprising 1 to 3 heteroatoms independently selected from N, O, S and P has been replaced with a C₁-C₆alkylene as defined herein.

As used herein, the term “C₁-C₆alkylene-aryl” refers to aryl as defined herein, wherein one of the hydrogen atoms of said aryl has been replaced with a C₁-C₆alkylene as defined herein.

As used herein, the term “C₁-C₆alkylene-5-6 membered heteroaryl comprising 1 or 2 heteroatoms independently selected from N, O and S” refers to a 5-6 membered heteroaryl comprising 1 or 2 heteroatoms independently selected from N, O and S as defined herein, wherein one of the hydrogen atoms of said 5-6 membered heteroaryl comprising 1 or 2 heteroatoms independently selected from N, O and S has been replaced with a C₁-C₆alkylene as defined herein.

As used herein, the term “C(═O)—C₁-C₆alkyl” refers to a C₁-C₆alkyl radical as defined herein, which is attached to the rest of the molecule via a C(═O) linker.

As used herein, the term “C(═O)—O—C₁-C₆alkyl” refers to a “O—C₁-C₆alkyl” radical as defined herein, which is attached to the rest of the molecule via a C(═O) linker.

As used herein, the term “C(═O)—O—C₁-C₆alkylene-O—C₁-C₆alkyl” refers to a “O—C₁-C₆alkylene-O—C₁-C₆alkyl” radical as defined herein, which is attached to the rest of the molecule via a C(═O) linker.

As used herein, the term “N(C₁-C₆alkyl)₂” refers to a radical which is connected to the rest of the molecule via the N atom, wherein the N atom is connected separately to two C₁-C₆ alkyl radicals as defined herein.

As used herein, the term “C(═O)—N(C₁-C₆alkyl)₂” refers to a radical of the formula “N(C₁-C₆alkyl)₂” as defined herein, which is attached to the rest of the molecule via a C(═O) linker.

As used herein, the term “NH(C₁-C₆alkyl)” refers to a radical which is connected to the rest of the molecule via the N atom, wherein the N atom is connected separately to i) a hydrogen atom, and ii) a C₁-C₆ alkyl radical as defined herein.

As used herein, the term “C(═O)NH(C₁-C₆alkyl)” refers to a radical of the formula “NH(C₁-C₆alkyl)” as defined herein, which is attached to the rest of the molecule via a C(═O) linker.

As used herein, the term “C(═O)—C₁-C₆alkylene-O—C₁-C₆alkyl” refers to a radical of the formula “C₁-C₆alkylene-O—C₁-C₆alkyl” as defined herein, which is attached to the rest of the molecule via a C(═O) linker.

As used herein, the term “C(═O)—C₃-C₈cycloalkyl” refers to “cycloalkyl” as defined herein having 3 to 8 ring atoms, which is attached to the rest of the molecule via a C(═O) linker.

As used herein, the term “C(═O)-3-10 membered heterocyclyl comprising 1 to 3 heteroatoms independently selected from N, O, S and P” refers to “heterocyclyl” as defined herein having 3 to 10 ring atoms, wherein from 1 to 3 of the ring atoms are heteroatoms selected from the group consisting of N, O, S and P (preferably 1 to 3 heteroatoms independently selected from N, O and S), which is attached to the rest of the molecule via a C(═O) linker.

As used herein, the term “C(═O)-aryl” refers to “aryl” as defined herein, and which is attached to the rest of the molecule via a C(═O) linker.

As used herein, the term “C(═O)-5-6 membered heteroaryl comprising 1 or 2 heteroatoms independently selected from N, O and S” refers to “5-6 membered heteroaryl” as defined herein, wherein said 5-6 membered heteroaryl contains 1 or 2 ring heteroatoms independently selected from the group consisting of N, O and S, and which is attached to the rest of the molecule via a C(═O) linker.

As used herein, the term “C₁-C₆alkylene-NR^{1A}R^{1B}” refers to a C₁-C₆alkyl radical as defined herein, wherein one of the hydrogen atoms of said C₁-C₆ alkyl radical has been replaced with NR^{1A}R^{1B}.

As used herein, a “NR^{1A}R^{1B}” substituent refers to a radical of the formula “N(R^{1A})(R^{1B})” wherein said radical is attached to the rest of the molecule by the nitrogen atom to which an R^{1A} group and an R^{1B} group are also separately attached, and wherein R^{1A} and R^{1B} may be the same or different, and are as defined herein.

As used herein, the term “C₁-C₆alkylene-[C₁-C₆alkylene-O—C₁-C₆alkyl]₂,” refers to a C₁-C₆alkyl radical as defined herein, wherein two hydrogen atoms of said C₁-C₆ alkyl radical have each independently been replaced with a C₁-C₆alkylene-O—C₁-C₆alkyl group as defined herein.

As used herein, the term “C₁-C₆alkylene-C(═O)—O—C₁-C₆alkyl” refers to a C₁-C₆alkyl radical as defined herein, wherein one of the hydrogen atoms of said C₁-C₆ alkyl radical has been replaced with a C(═O)—O—C₁-C₆alkyl radical as defined herein.

As used herein, the term “C(═O)—C₃-C₈cycloalkyl” refers to a C₃-C₈cycloalkyl as defined herein and which is attached to the rest of the molecule via a C(═O) linker.

As used herein, the term “C(═O)—C₁-C₆haloalkyl” refers to a C₁-C₆haloalkyl as defined herein and which is attached to the rest of the molecule via a C(═O) linker.

As used herein, the term “C(═O)—C₁-C₆hydroxyalkyl” refers to a C₁-C₆hydroxyalkyl as defined herein and which is attached to the rest of the molecule via a C(═O) linker.

As used herein, the term “C₃-C₈hydroxycycloalkyl” refers to a C₃-C₈cycloalkyl as defined herein wherein one or more of the hydrogen atoms of the C₃-C₈cycloalkyl have been replaced by hydroxyl. As will be appreciated by one of normal skill in the art, only one hydrogen atom on any given carbon atom in said C₃-C₈cycloalkyl can be replaced by hydroxyl.

As used herein, the term “C₁-C₆alkylene-C₃-C₈hydroxycycloalkyl” refers to C₁-C₆alkyl as defined herein wherein one of the hydrogen atoms of said C₁-C₆ alkyl radical has been replaced with C₃-C₈hydroxycycloalkyl as defined herein.

As used herein, the term “substituent” refers to a radical group which replaces a hydrogen atom in a given molecule.

As used herein, the term “substituted by one or more substituents” includes substituted by 1, 2, 3, 4, 5, or 6 substituents. Preferably, it includes 1 substituent or 2 or 3 substituents. For the avoidance of doubt, this term also includes instances where 2 or 3 substituents may be present on the same carbon atom where valency allows. When multiple substituents are present, the substituents are selected independently unless otherwise indicated, so where 2 or 3 substituents are present, for example, those substituents may be the same or different.

As used herein, the term “C₁-C₆alkylene” refers to a straight-chain or branched divalent radical of an alkyl group having from 1 to 6 carbon atoms e.g., —CH₂—, —CH₂CH₂—, and —CH₂CH₂CH₂—.

As used herein, the term “O—C₁-C₆alkylene” or, equivalently “C₁-C₆alkylene-O” refers to C₁-C₆alkyl as defined herein wherein one of the hydrogen atoms of said C₁-C₆alkyl has been replaced with O.

As used herein, the term “C(═O)—C₁-C₆alkylene” or, equivalently, “C₁-C₆alkylene-C(═O)” refers to C₁-C₆alkyl as defined herein wherein one of the hydrogen atoms of said C₁-C₆alkyl has been replaced with C(═O).

As used herein, the term “C(═O)—O—C₁-C₆alkylene” or, equivalently, “C₁-C₆alkylene-O—C(═O)” refers to O—C₁-C₆alkylene as defined herein connected to the remainder of the molecule by a C(═O) linking group

As used herein, the term “C₁-C₆alkylene-C(═O)-3-10 membered heterocyclyl” refers to C(═O)-3-10 membered heterocyclyl as defined herein connected to the remainder of the molecule by a C₁-C₆alkylene linking group.

As used herein, the term “bridging group” refers to a mutual, connecting substituent of non-adjacent bridgehead ring atoms in a cyclic structure. For example, in the following structure

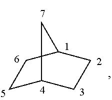

the “bridgehead ring atoms” are represented by 1 and 4, and the methylene group represented by 7 could be said to be the “bridging group”. In this situation, as used herein, the bridging group forms a C₅cycloalkyl (i.e a 5-membered carbocyclic ring formed from atoms 1, 2, 3, 4 and 7 or formed from atoms 1, 4, 5, 6, 7).

As such, one example of a bridging group formed by two substituents on non-neighboring bridgehead ring atoms is methylene. In an alternative example, the “bridging group” is ethylene. In an additional example, the “bridging group” is C═O. In a yet alternative example, it is CH₂—C(═O). Preferably, the “bridging group” is methylene or ethylene.

When a (secondary) ring is formed by two substituents on neighboring (primary) ring atoms, the (secondary) ring size is expressed herein as the total number of (secondary) ring atoms, including the two primary ring atoms. For example, taking the following structure:

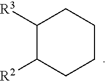

Here, where R² and R³ together form a C₅cycloalkyl, the resulting structure would be

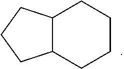

Likewise, when a (secondary) ring is formed by two substituents on the same (primary) ring atom, the (secondary) ring size is expressed herein as the total number of (secondary) ring atoms, including the primary ring atom. For example, taking the following structure:

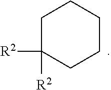

Here, where the two R² groups together form a C₅cycloalkyl, the resulting structure would be

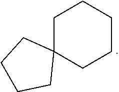

The term “fused bicyclic heteroaryl group” refers to a group comprising two rings joined through the sharing of one covalent bond, wherein the fused group is aromatic in nature (i.e. follows Huckel's rule) and comprises one to three ring heteroatoms selected from the group consisting of N, O and S. The fused bicyclic heteroaryl group is bonded to the remainder of the molecule through a carbon atom. An example of a fused bicyclic heteroaryl group is indole. For instance,

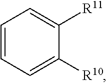

where R¹⁰ and R¹¹ are joined together to form a 9 membered fused bicyclic aryl group, can be, as a (non-limiting) example,

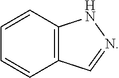

The term “fused bicyclic aryl group” refers to a group comprising two rings joined through the sharing of one covalent bond, wherein the fused group is aromatic in nature (i.e. follows Huckel's rule) and comprises no heteroatoms. The fused bicyclic heteroaryl group is bonded to the remainder of the molecule through a carbon atom. An example of a fused bicyclic aryl group is naphthalene.

As used herein, the term “N(C₁-C₆alkyl)-C(═O)—C₁-C₆alkyl” refers to NH(C₁-C₆alkyl) as defined herein wherein the hydrogen atom directly attached to the N is replaced with a C(═O)—C₁-C₆alkyl group as defined herein.

As used herein, the term “N(C₁-C₆alkyl)-3-10 membered heterocyclyl” refers to NH(C₁-C₆alkyl) as defined herein wherein the hydrogen atom directly attached to the N is replaced with a 3-10 membered heterocyclyl as defined herein.

As used herein, the term “N(C₁-C₆alkyl)-C(═O)—C₁-C₆alkylene-O—C₁-C₆alkyl” refers to NH(C₁-C₆alkyl)₂ as defined herein wherein the hydrogen atom directly attached to the N is replaced with C(═O)—C₁-C₆alkylene-O—C₁-C₆alkyl as defined herein.

As used herein, the term “3-10 membered heterocyclyl-O—C₁-C₆alkyl” refers to 3-10 membered heterocyclyl as used herein wherein a hydrogen atom in said 3-10 membered heterocyclyl has been replaced with O—C₁-C₆alkyl as used herein.

As used herein, the term “N(C₁-C₆alkylene-O—C₁-C₆alkyl)₂” refers to a radical which is connected to the rest of the molecule via the N atom, wherein the N atom is connected separately to two C₁-C₆alkylene-O—C₁-C₆alkyl radicals as defined herein.

As used herein, the term “perdeuterated” means that all hydrogen atoms are replaced with deuterium.

The term “co-crystal” as used herein refers to a single crystalline phase comprising a plurality of different molecular or ionic compounds which are neither solvates nor simple salts.

The term “solvate” as used herein refers to a single crystalline phase (which may comprise a single molecular or ionic compound, or could equally be a co-crystal) containing disordered, partially ordered, or ordered solvent molecules, preferably wherein the solvent molecules are partially ordered or ordered. The solvent molecule can be water, in which case the “solvate” can also be referred to as a “hydrate”.

Depending on the choice of the starting materials and procedures, the compounds can be present in the form of one of the possible stereoisomers or as mixtures thereof, for example as pure optical isomers, or as stereoisomer mixtures, such as racemates, diastereoisomer and/or atropisomer mixtures, depending on the number of asymmetric carbon atoms. The present invention is, unless specified otherwise, meant to include all such possible stereoisomers, including racemic mixtures, diasteriomeric and atropisomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto. When both a basic group and an acid group are present in the same molecule, the compounds of the present invention may also form internal salts, e.g., zwitterionic molecules. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases. Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine. In another aspect, the present invention provides compounds of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

As used herein, the term nitrogen protecting group (PG) in a compound described herein and also as depicted in the Schemes, refers to a group that should protect the functional groups concerned against unwanted secondary reactions, such as acylations, etherifications, esterifications, oxidations, solvolysis and similar reactions. It may be removed under deprotection conditions. Depending on the protecting group employed, the skilled person would know how to remove the protecting group to obtain the free amine NH₂ group by reference to known procedures. These include reference to organic chemistry textbooks and literature procedures such as J. F. W. McOmie, “Protective Groups in Organic Chemistry”, T. W. Greene and P. G. M. Wuts, “Greene's Protective Groups in Organic Synthesis”, and in “Methoden der organischen Chemie” (Methods of Organic Chemistry).

The term “stereoisomer” or “stereoisomers” refer to compounds, which have identical chemical constitution, but differ with regard to the arrangement of the atoms or groups in space.

The term “diastereoisomer” or “diastereomer” refers to stereoisomers not related as mirror images. Diastereoisomers are characterized by differences in physical properties, and by some differences in chemical behaviour. Mixtures of diastereomers may separate under analytical procedures such as chromatography or crystallisation.

The term “enantiomer” refers to one of a pair of molecular entities which are mirror images of each other and non-superimposable.

The term “enantiomeric mixture” refers to an enantiomerically enriched mixture, a composition that comprises a greater proportion or percentage of one of the enantiomers of the compounds of the invention, in relation to the other enantiomer, or a racemate.

The term “diastereomeric mixture” refers to a diastereomerically enriched mixture or a mixture of diastereoisomers of equal proportion.

The term “diastereomerically enriched” refers to a composition that comprises a greater proportion or percentage of one of the diastereomers of the compounds of the invention, in relation to the other diastereoisomer(s).

Where the absolute stereochemistry of the compounds, for instance in the Examples, is specified, the Cahn-Ingold-Prelog system is used for assigning stereochemical descriptors to the examples.

Any formula given herein is also intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen. Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium. Other examples of isotopes that can be incorporated into compounds of the invention include isotopes of hydrogen, carbon, nitrogen, oxygen, phosphorous, fluorine, and chlorine, such as ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. Accordingly, it should be understood that the invention includes compounds that incorporate one or more of any of the aforementioned isotopes, including for example, radioactive isotopes, such as ³H and ¹⁴C, or those into which non-radioactive isotopes, such as ²H (D) and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagent& in place of the non-labeled reagent previously employed. In general, the atoms described in formula (I) are not intended to be limited to particular isotopic forms. As used herein, the term “hydrogen” or “H” is intended to cover both “¹H” and “²H”/“D” (and indeed even “³H”). On the contrary, the term “deuterium” or “D” refers specifically to “²H”. In one aspect the hydrogens in the compound of Formula (I) are present in their normal isotopic abundances. In a another embodiment, some or all the hydrogens are isotopically enriched in deuterium (D).

As used herein, the term “hydrogen” or “H” is intended to cover both “¹H” and “²H”, i.e. “D” (and indeed even “³H”). The term “deuterium” or “D” refers specifically to “²H”. In one aspect the hydrogens in the compound of Formula (I) are present in their normal isotopic abundances. In a another embodiment, some or all the hydrogens are isotopically enriched in deuterium (D).

As used herein, the term “pharmaceutical composition” refers to a compound of the invention, or a pharmaceutically acceptable salt thereof, together with at least one pharmaceutically acceptable carrier, in a form suitable for oral or parenteral administration. As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22^{nd} Ed. Pharmaceutical Press, 2013, pp. 1049-1070).

In one aspect, the present invention provides a pharmaceutical composition comprising a compound of the present invention, or a pharmaceutically acceptable salt thereof, and a pharmaceutically acceptable carrier. In one embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

In an embodiment, the pharmaceutical compositions are capsules comprising the active ingredient only.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs, solutions or solid dispersion. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a pro-longed period of time, and means to secure the device to the skin.

Suitable compositions for topical application, e.g., to the skin and eyes, include aqueous solutions, suspensions, ointments, creams, gels or sprayable formulations, e.g., for delivery by aero-sol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The compounds of formula (I) in free form or in pharmaceutically acceptable salt form, exhibit valuable pharmacological properties, e.g. RAS-mutant inhibiting properties, e.g. as indicated in the in vitro tests as provided in the examples, and are therefore indicated for therapy or for use as research chemicals, e.g. as tool compounds.

Particularly interesting compounds of the invention have good potency in the biological assays described herein, in particular in the covalent competition assay as described herein. In another aspect, they should have a favourable safety profile. In another aspect, they should possess favourable pharmacokinetic properties.

Compounds of the present invention preferably have an IC 50 of less than 0.5 uM, more preferably of less than 0.1 uM.

Having regard to their activity as RAS mutant inhibitors, in particular, KRAS, HRAS or NRAS G12C mutant inhibitors, compounds of the formula (I) in free or pharmaceutically acceptable salt form, are useful in the treatment of conditions which are driven by KRAS, HRAS or NRAS G12C mutations, such as a cancer that is responsive (meaning especially in a therapeutically beneficial way) to inhibition of RAS mutant proteins, in particular, KRAS, HRAS or NRAS G12C mutant proteins, most especially a disease or disorder as mentioned herein below.

The pharmaceutical composition or combination of the present invention may, for example, be in unit dosage of about 1-1000 mg of active ingredient(s) for a subject of about 50-70 kg.

Compounds of the invention may be useful in the treatment of cancer. In particular, the compounds of the invention may be useful in the treatment of an indication which is selected from the group consisting of lung cancer (such as lung adenocarcinoma and non-small cell lung cancer), colorectal cancer (including colorectal adenocarcinoma), pancreatic cancer (including pancreatic adenocarcinoma), uterine cancer (including uterine endometrial cancer), rectal cancer (including rectal adenocarcinoma) and solid tumors.

The compounds of the invention may also be useful in the treatment of solid malignancies characterized by mutations of RAS.

The compounds of the invention may also be useful in the treatment of solid malignancies characterized by mutations of KRAS, in particular G12C mutations in KRAS.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc.

As used herein, the term “subject” refers to primates (e.g., humans, male or female), dogs, rabbits, guinea pigs, pigs, rats and mice. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”, “for example”, “optionally” or “preferably”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Typically, the compounds of formula (I) can be prepared according to the Schemes provided herein. The examples which outline specific synthetic routes, and the generic schemes below provide guidance to the synthetic chemist of ordinary skill in the art, who will readily appreciate that the solvent, concentration, reagent, protecting group, order of synthetic steps, time, temperature, and the like can be modified as necessary.

The compound of the present invention may be administered either simultaneously with, or before or after, one or more other therapeutic agent. The compound of the present invention may be administered separately, by the same or different route of administration, or together in the same pharmaceutical composition as the other agents. A therapeutic agent is, for example, a chemical compound, peptide, antibody, antibody fragment or nucleic acid, which is therapeutically active or enhances the therapeutic activity when administered to a patient in combination with a compound of the present invention. In embodiments of the invention, the other therapeutic agent may be an anti-cancer agent.

In one embodiment, the invention provides a product comprising a compound of the present invention and at least one other therapeutic agent as a combined preparation for simultaneous, separate or sequential use in therapy. In one embodiment, the therapy is the treatment of a disease or condition characterized by a KRAS HRAS or NRAS G12C mutation. Products provided as a combined preparation include a composition comprising the compound of the present invention and the other therapeutic agent(s) together in the same pharmaceutical composition, or the compound of the present invention and the other therapeutic agent(s) in separate form, e.g. in the form of a kit.

In one embodiment, the invention provides a pharmaceutical composition comprising a compound of the present invention and another therapeutic agent(s). Optionally, the pharmaceutical composition may comprise a pharmaceutically acceptable carrier, as described above.

In one embodiment, the invention provides a kit comprising two or more separate pharmaceutical compositions, at least one of which contains a compound of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

The pharmaceutical composition or combination of the present invention may, for example, be in unit dosage of about 1-1000 mg of active ingredient(s) for a subject of about 50-70 kg.

In the combination therapies of the invention, the compound of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

The schemes provided herein are intended to represent single diastereomers/enantiomers as well as their isomeric mixtures. Separation of diastereomers/enantiomers may be performed according to techniques described herein.

The amine protecting group (also referred to herein as nitrogen protecting group) is referred to as “PG” in the Schemes below.

### Schemes

### Preparation of Compounds

Several methods for preparing the compounds of this disclosure are described in the following schemes. Starting materials and intermediates are purchased, made from known procedures, or prepared as otherwise illustrated. In some cases, the order of carrying out the steps of reaction schemes may be varied to facilitate the reaction or to avoid unwanted reactions. The R groups and other variables in the Schemes correspond to those defined in Formula (I).

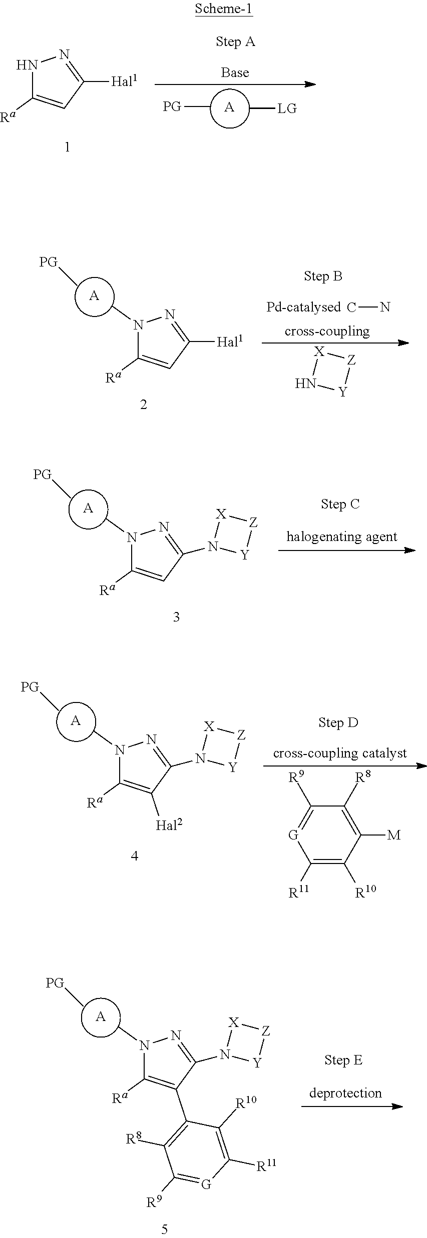

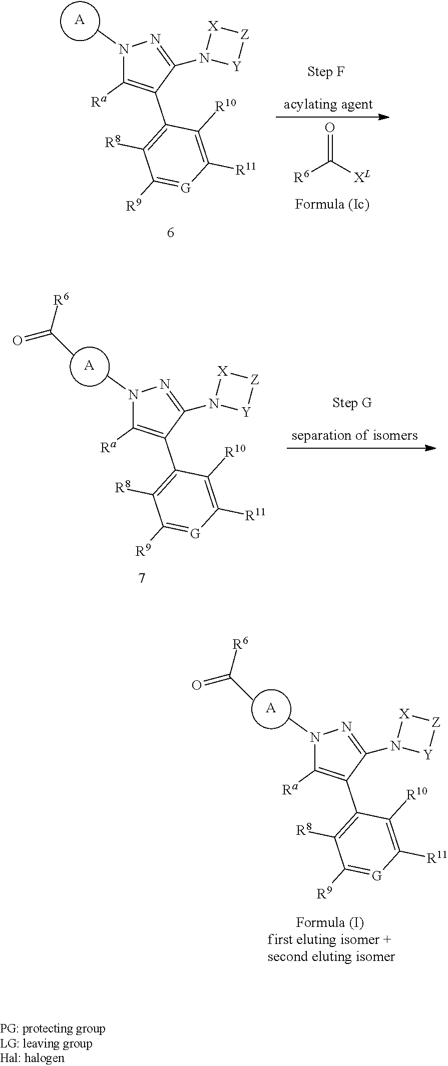

Scheme-1: A compound of Formula (I) as disclosed herein can be synthesised as outlined in Scheme-1. In step A, an appropriate halogenated heteroaromatic (1) such as 3-iodo-5-methyl-1H-pyrazole is alkylated with a suitably functionalized N-protected linker, for example with a tosylate in the presence of a base such as cesium carbonate in a solvent such as DMF to provide the halogenated heteroaromatic compound (2). In step B, compound (2) is reacted with a secondary amine coupling partner in a metal-catalysed carbon-nitrogen cross-coupling reaction in the presence of a palladium catalyst such as tBuXPhos-Pd-G3 or Pd(dba)2 and bis(3,5-bis(trifluoromethyl)phenyl)(2′,6′-bis(dimethylamino)-3,6-dimethoxybiphenyl-2-yl)phosphine (CAS: 1810068-30-4) in a solvent such as 1,4-dioxane (or toluene) with a base such as NaOtBu (or Phosphazene P₂-Et) to provide compound (3). In step C, compound (3) is treated with a halogenating agent such as N-iodosuccinimide or N-bromosuccinimide in a solvent such as THF or CH₃CN. In step D, the heteroaromatic substituent is introduced with a palladium cross-coupling reaction, using a suitably functionalized aryl or heteroaryl system, for example an heteroaryl boronate ester in the presence of a palladium catalyst such as RuPhos-Pd-G3/RuPhos or CataCXium-A-Pd-G3 (CAS: 1651823-59-4) in a solvent such as dioxane (or toluene or CPME) with a base such as K₃PO₄ (or KOH) to provide compound (5). Alternatively, compound (5) can also be prepared by performing Step C (with NBS) before Step B and then proceeding as described above. In step E, the protecting groups (PG) are removed under appropriate conditions. For example, the Boc group of compound (5) is removed using conditions known in the art, with an organic acid such as trifluoroacetic acid in a solvent such as dichloromethane or with a mineral acid such as sulfuric acid in a solvent such as 1,4-dioxane to provide compound (6). The heteroaryl introduced in Step D may also contain a protecting group (for example, THP) which is removed in the same reaction under the aforementioned conditions for cleaving the Boc group. In step F, compound (7) may be made by reaction of compound (6) with a compound of Formula (Ic) where X^{L} is a leaving group, for example halo (such as chloro) in the presence of a suitable base (such as Hunig's base); or where X^{L} is OH and the reaction is carried out under standard amide bond forming conditions for example in the presence of an amide coupling reagent such as HATU and a suitable base such DIPEA. For example, the acrylamide is introduced by treating compound (6) with acrylic acid in presence of a coupling agent such as propylphosphonic anhydride and a base such as DIPEA in a solvent such as methylene chloride to provide compound (7). Alternatively compound (6) can be treated with acryloyl chloride in the presence of a base such as aqueous sodium bicarbonate in a solvent such as THF. In step G, the mixture of atropisomers is separated using SFC or HPLC conditions with the appropriate column and eluent.

Compounds (1), (2), (3), (4), (5), (6) and (7) as shown and described above for Scheme-1 are useful intermediates for preparing compounds of Formula (I). In compounds (1), (2), (3), (4), (5), (6) and (7) of Scheme 1, Ring A, R^{a}, R⁶, R⁸, R⁹, R¹⁰, R¹¹, G, X, Y and Z are defined according to any one of enumerated Embodiments 1 to 55.

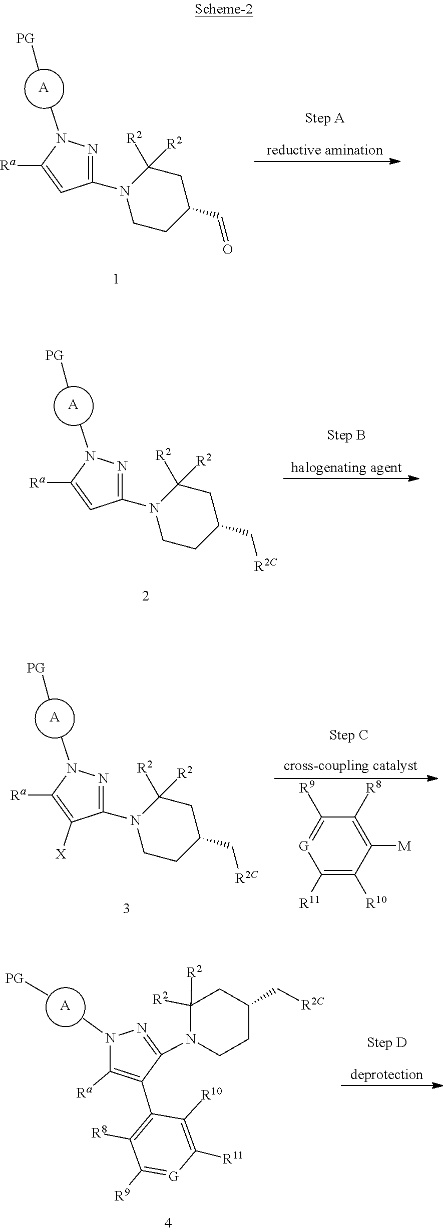

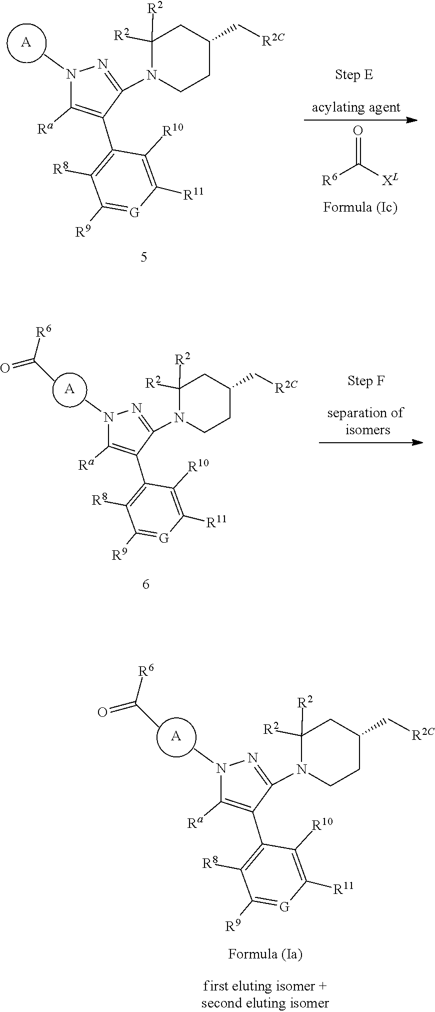

Scheme-2: Scheme-2 provides an alternative method for preparation of compounds of Formula (Ia) as disclosed herein. In step A, the aldehyde (1) is coupled with an amine to provide compound (2). This reductive amination proceeds in the presence of a reducing agent such as sodium triacetoxyborohydride. The remaining steps B-F are analogous to steps C-G in Scheme-1 described above.

Compounds (1), (2), (3), (4), (5) and (6) as shown and described above for Scheme-2 are useful intermediates for preparing compounds of Formula (Ia). In compounds (1), (2), (3), (4), (5) and (6) of Scheme 2, Ring A, R^{a}, R⁶, R⁸, R⁹, R¹⁰, R¹¹, G, X, Y and Z are defined according to any one of enumerated Embodiments 1 to 55.

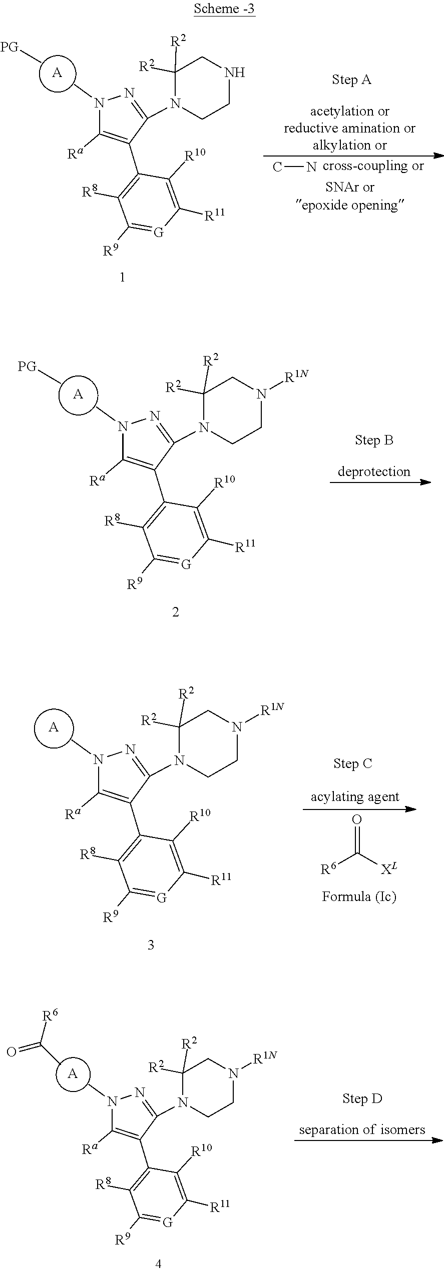

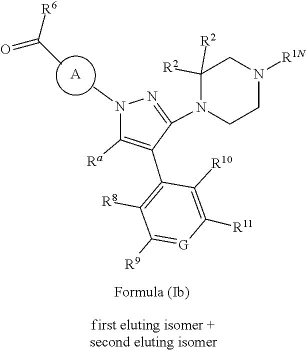

Scheme-3: Scheme-3 provides an alternative method for preparation of a variety of compounds of Formula (Ib) as disclosed herein starting from a common amine compound (1). In one approach, amine (1) is reacted with an activated acid using a coupling reagent such as HATU in presence of base such as DIPEA in an inert solvent such as DMF to form an amide (2). In another approach, amine (1) is coupled to an aldehyde under reductive amination conditions in the presence of a reducing agent such as sodium triacetoxyborohydride. Amine (1) can also be reacted with an alkylating agent such as with (S)-(1,4-dioxan-2-yl)methyl 4-methylbenzenesulfonate in presence of Et₃N in a solvent such as DMF; with an oxirane in presence of LiClO₄ in DMF; with methyl alpha-bromoisobutyrate in presence of a base such as Cs₂CO₃ in a solvent such as DMF; and with 3-((phenylsulfonyl)methylene)oxetane in a solvent such as MeOH. In those cases where the alkylating agent contains a further reactive group such as an ester or a phenylsulfonyl group, an additional reaction (e.g. ester reduction with LiBH₄ in THF or desulfonylation with magnesium) may be performed before proceeding with Step B. In another approach, arylation of amine (1) in a metal-catalysed carbon-nitrogen cross-coupling reaction in the presence of a palladium catalyst such as Pd(dba)₂ and bis(3,5-bis(trifluoromethyl)phenyl)(2′,6′-bis(dimethylamino)-3,6-dimethoxybiphenyl-2-yl)phosphine (CAS: 1810068-30-4) in a solvent such as 1,4-dioxane with a base such as NaOtBu to provide compound (2). Alternatively, amine (1) is reacted with aryl or heteroaryl halide in presence of a base such as Et₃N in a solvent such as EtOH. The remaining steps B-D are analogous to steps E-G in Scheme-1 described above.

Compounds (1), (2), (3) and (4) as shown and described above for Scheme-2 are useful intermediates for preparing compounds of Formula (Ib). In compounds (1), (2), (3) and (4) of Scheme 2, Ring A, R^{a}, R⁶, R⁸, R⁹, R¹⁰, R¹¹, G, X, Y and Z are defined according to any one of enumerated Embodiments 1 to 55.

### Preparation of Compounds

Compounds of the present invention can be prepared as described in the following Examples. Which are intended to illustrate the invention and are not to be construed as being limitations thereof. All starting materials, building blocks, reagents, acids, bases, dehydrating agents, solvents, and catalysts utilized to prepare the compounds of the present invention are either commercially available or can be produced by organic synthesis methods known to one of ordinary skill in the art. Furthermore, the compounds of the present invention can be produced by organic synthesis methods known to one of ordinary skill in the art as shown in the following examples.

The structures of all final products, intermediates and starting materials are confirmed by standard analytical spectroscopic characteristics, e.g., MS, IR, NMR. The absolute stereochemistry of representative examples of the preferred (most active) atropisomers has been determined by analyses of X-ray crystal structures of complexes in which the respective compounds are bound to the KRasG12C mutant. In all other cases where X-ray structures are not available, the stereochemistry has been assigned by analogy, assuming that, for each pair, the atropoisomer exhibiting the highest activity in the covalent competition assay has the same configuration as observed by X-ray. Where the absolute stereochemistry of the compounds in the Examples is specified, the Cahn-Ingold-Prelog system is used for assigning stereochemical descriptors to the examples.

In embodiments of the invention, compounds of formula (I) have an absolute configuration equivalent to the one depicted for Example 121b. As illustrated below, the absolute stereochemistry is assigned according to the Cahn-Ingold-Prelog rule. In this case, Example 121b (most active atropisomer) has the a(R) configuration and Example 121a (less active atropisomer) the a(S) configuration.

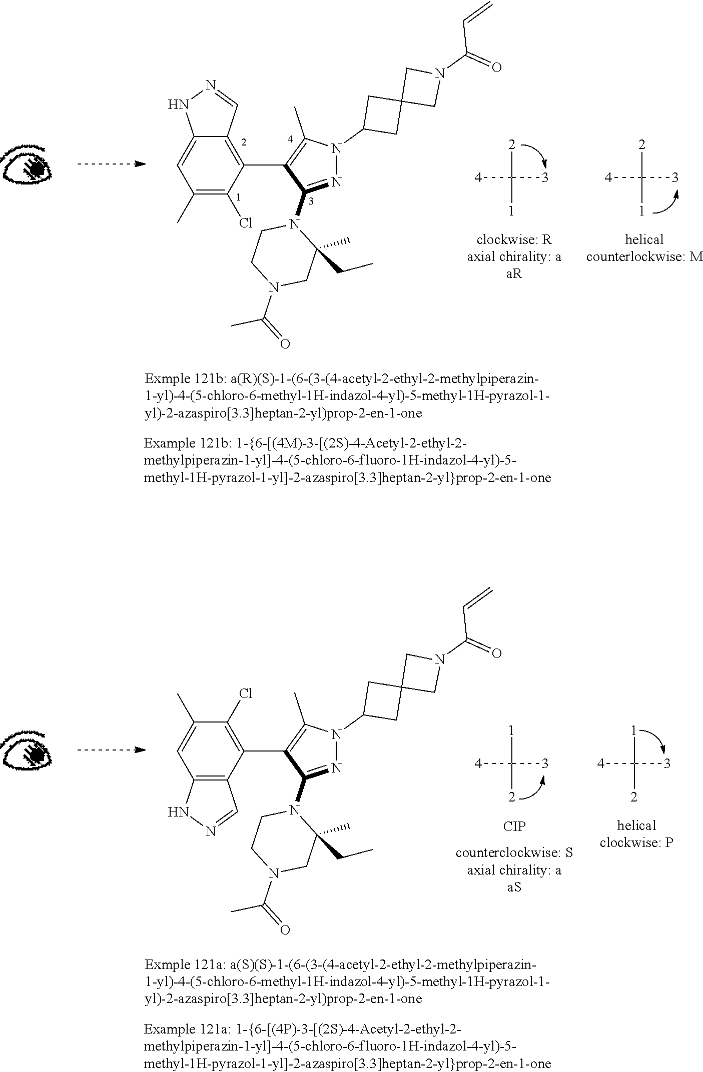

### General Conditions and Instrumentation

Microwave: All microwave reactions were conducted in a Biotage Initiator, irradiating at 0-400 W from a magnetron at 2.45 GHz with Robot Eight/Robot Sixty processing capacity, unless otherwise stated.

Mass spectra were acquired on LC-MS, SFC-MS, or GC-MS systems using electrospray, chemical and electron impact ionization methods with a range of instruments of the following configurations: Waters Acquity UPLC with Waters SQ detector. [M+H]⁺ refers to the protonated molecular ion of the chemical species.

NMR spectra were run with Bruker Ultrashield™400 (400 MHz), Bruker Ultrashield™600 (600 MHz) and Bruker Ascend™400 (400 MHz) spectrometers, both with and without tetramethylsilane as an internal standard. Chemical shifts (□-values) are reported in ppm downfield from tetramethylsilane, spectra splitting pattern are designated as singlet (s), doublet (d), triplet (t), quartet (q), multiplet, unresolved or more overlapping signals (m), broad signal (br). Solvents are given in parentheses. Only signals of protons that are observed and not overlapping with solvent peaks are reported.

Temperatures are given in degrees Celsius. If not mentioned otherwise, all evaporations are performed under reduced pressure, typically between about 15 mm Hg and 100 mm Hg (=20-133 mbar).
Celite: Celite^{R} (the Celite corporation)=filtering aid based on diatomaceous earth
Phase separator: Biotage-Isolute phase separator-(Part number: 120-1908-F for 70 mL and part number: 120-1909-J for 150 mL)
SiliaMetS®Thiol: SiliCYCLE thiol metal scavenger-(R51030B, Particle Size: 40-63 μm)
Si-TMT: TCl-2,4,6-trimercaptotriazine Silica Gel-(S0865) Cas 1226494-16-1

### UPLC-MS and MS Analytical Methods:

UPLC-MS-1a: Acquity HSS T3; particle size: 1.8 μm; column size: 2.1×50 mm; eluent A: H₂O+0.05% HCOOH+3.75 mM ammonium acetate; eluent B: CH₃CN+0.04% HCOOH; gradient: 5 to 98% B in 1.40 min then 98% B for 0.40 min; flow rate: 1 mL/min; column temperature: 60° C.

UPLC-MS-1b: Acquity HSS T3; particle size: 1.8 μm; column size: 2.1×50 mm; eluent A: H₂O+0.05% HCOOH+3.75 mM ammonium acetate; eluent B: CH₃CN+0.04% HCOOH; gradient: 5 to 98% B in 9.40 min then 98% B for 0.40 min; flow rate: 1 mL/min; column temperature: 60° C.

UPLC-MS-1c: Acquity HSS T3; particle size: 1.8 μm; column size: 2.1×50 mm; eluent A: H₂O+0.05% HCOOH+3.75 mM ammonium acetate; eluent B: CH₃CN+0.04% HCOOH; gradient: 5 to 98% B in 9.40 min then 98% B for 0.40 min; flow rate: 0.8 mL/min; column temperature: 50° C.

UPLC-MS-1d: Acquity HSS T3; particle size: 1.8 μm; column size: 2.1×100 mm; eluent A: H₂O+0.05% HCOOH+3.75 mM ammonium acetate; eluent B: CH₃CN+0.04% HCOOH; gradient: 5 to 98% B in 9.40 min then 98% B for 0.40 min; flow rate: 0.8 mL/min; column temperature: 60° C.

UPLC-MS-1e: Acquity HSS T3; particle size: 1.8 μm; column size: 2.1×50 mm; eluent A: H₂O+0.05% HCOOH+3.75 mM ammonium acetate; eluent B: isopropanol+0.05% HCOOH; gradient: 5 to 98% B in 1.7 min then 98% B for 0.10 min; flow rate: 0.6 mL/min; column temperature: 80° C.

UPLC-MS-1f: Acquity HSS T3; particle size: 1.8 μm; column size: 2.1×100 mm; eluent A: H₂O+0.05% HCOOH+3.75 mM ammonium acetate; eluent B: CH₃CN+0.05% HCOOH; gradient: 5 to 60% B in 8.40 min then 98% B for 1 min; flow rate: 0.4 mL/min; column temperature: 80° C.

UPLC-MS-1q: Acquity HSS T3; particle size: 1.8 μm; column size: 2.1×50 mm; eluent A: H₂O+0.05% HCOOH+3.75 mM ammonium acetate; eluent B: isopropanol+0.05% HCOOH; gradient: 5 to 98% B in 1.7 min then 98% B for 0.10 min; flow rate: 0.7 mL/min; column temperature: 80° C.

UPLC-MS-1h: Acquity HSS T3; particle size: 1.8 μm; column size: 2.1×100 mm; eluent A: H₂O+0.05% HCOOH+3.75 mM ammonium acetate; eluent B: CH₃CN+0.04% HCOOH; gradient: 5 to 98% B in 9.40 min then 98% B for 0.40 min; flow rate: 1 mL/min; column temperature: 60° C.

UPLC-MS-2a: Acquity BEH C18; particle size: 1.7 μm; column size: 2.1×50 mm; eluent A: H₂O+4.76% isopropanol+0.05% HCOOH+3.75 mM ammonium acetate; eluent B: isopropanol+0.05% HCOOH; gradient: 1 to 98% B in 1.70 min then 98% B for 0.10 min; flow rate: 0.6 mL/min; column temperature: 80° C.

UPLC-MS-2b: Acquity BEH C18; particle size: 1.7 μm; column size: 2.1×50 mm; eluent A: H₂O+0.05% HCOOH+3.75 mM ammonium acetate; eluent B: isopropanol+0.05% HCOOH; gradient: 5 to 98% B in 1.70 min then 98% B for 0.10 min; flow rate: 0.6 mL/min; column temperature: 80° C.

UPLC-MS-2c: Basic XBridge BEH C18; particle size: 2.5 μm; column size: 2.1×50 mm; eluent A: H₂O+5 mM NH₄OH; eluent B: CH₃CN+5 mM NH₄OH; gradient: 2 to 98% B in 1.40 min then 98% B for 0.60 min; flow rate: 1 mL/min; column temperature: 50° C.

UPLC-MS-2d: Acquity BEH C18; particle size: 1.7 μm; column size: 2.1×100 mm; eluent A: H₂O+0.05% HCOOH+3.75 mM ammonium acetate; eluent B: isopropanol+0.05% HCOOH; gradient: 5 to 60% B in 8.40 min then 60 to 98% B in 1 min; flow rate: 0.4 mL/min; column temperature: 80° C.

UPLC-MS-2e: Acquity BEH C18; particle size: 1.7 μm; column size: 2.1×100 mm; eluent A: H₂O+4.76% isopropanol+0.05% HCOOH+3.75 mM ammonium acetate; eluent B: isopropanol+0.05% HCOOH; gradient: 1 to 60% B in 8.40 min then 60 to 98% B in 1 min; flow rate: 0.4 mL/min; column temperature: 80° C.

UPLC-MS-2f: Acquity BEH C18; particle size: 1.7 μm; column size: 2.1×50 mm; eluent A: H₂O+0.05% HCOOH+3.75 mM ammonium acetate; eluent B: isopropanol+0.05% HCOOH; gradient: 5 to 98% B in 1.70 min then 98% B for 0.10 min; flow rate: 0.7 mL/min; column temperature: 80° C.

UPLC-MS-3: Ascentis Express C18; particle size: 2.7 μm; column size: 2.1×50 mm; eluent A: H₂O+4.76% isopropanol+0.05% HCOOH+3.75 mM ammonium acetate; eluent B: isopropanol+0.05% HCOOH; gradient: 1 to 50% B in 1.40 min, 50 to 98% B in 0.30 min, then 98% for 0.10 min; flow rate: 1 mL/min; column temperature: 80° C.

UPLC-MS-4: CORTECS C18+; particle size: 2.7 μm; column size: 2.1×50 mm; eluent A: H₂O+4.76% isopropanol+0.05% HCOOH+3.75 mM ammonium acetate; eluent B: isopropanol+0.05% HCOOH; gradient: 1 to 50% B in 1.40 min, 50 to 98% B in 0.30 min, then 98% for 0.10 min; flow rate: 1 mL/min; column temperature: 80° C.

UPLC-MS-5: Acquity BEH C18; particle size: 1.7 μm; column size: 2.1×50 mm; eluent A: H₂O+0.10% HCOOH+2.0 mM ammonium acetate; eluent B: CH₃CN+0.10% HCOOH; gradient: 98:2 at 0.01 min up to 0.3 min, 50:50 at 0.6 min, 25:75 at 1.1 min, 0:100 at 2.0 min up to 2.70 min at flow rate: 0.60 mL/min, 98:2 at 2.71 min up to 3.0 min at Flow rate: 0.55 mL/min; column temperature: RT.

UPLC-MS-6: Acquity BEH C18; particle size: 1.7 μm; column size: 2.1×50 mm; eluent A: H₂O+0.10% HCOOH+2.0 mM ammonium acetate; eluent B: CH₃CN+0.10% HCOOH; gradient 50:50 at 0.01 min, 10:90 at 1.0 min, 0:100 at 1.5 min up to 4.50 min, 50:50 at 4.6 min up to 5.0 min; flow rate: 0.40 mL/min; column temperature: RT.

UPLC-MS-7: Acquity BEH C18; particle size: 1.7 μm; column size: 2.1×50 mm; eluent A: H₂O+0.10% HCOOH+2.0 mM ammonium acetate; eluent B: CH₃CN+0.1% HCOOH; gradient: 98:2 at 0.01 min up to 0.5 min, 10:90 at 5.0 min, 5:95 at 6.0 min until 7.0 min, 98:2 at 7.01 min up to 8.0 min; flow rate: 0.45 mL/min; column temperature: RT.

UPLC-MS-8: Acquity BEH C18; particle size: 1.7 μm; column size: 2.1×50 mm; eluent A: H2O+0.1% HCOOH+2.0 mM ammonium acetate/CH₃CN (90:10); eluent B: CH₃CN+0.10% HCOOH; gradient: 95:5 at 0.01 min, 0:100 at 0.40 min, 0:100 at 0.50 min (flow rate: 0.65 mL/min), 0:100 at 1.30 min (flow rate: 0.70 mL/min), 95:5 at 1.31 min up to 1.50 min (flow rate: 0.60 mL/min); column temperature: RT.

UPLC-MS-9: Acquity BEH C18; particle size: 1.7 μm; column size: 2.1×50 mm; eluent A: 2 mM ammonium acetate+0.1% HCOOH in water; eluent B: 0.1% HCOOH in CH₃CN; gradient: 95:5 at 0.01 min (flow rate: 0.55 mL/min), 30:70 at 0.60 min (flow rate: 0.60 mL/min), 10:90 at 0.80 min (flow rate: 0.65 mL/min, 0:100 at 1.10 min up to 1.70 min (flow rate: 0.65 mL/min), 95:5 at 1.71 min up to 2.0 min (flow rate: 0.55 mL/min); column temperature: RT.

UPLC-MS-10: Waters, YMC Triart C18; particle size: 5 μm; column size: 150×4.6 mm; eluent A: 10 mM ammonium acetate; eluent B: CH₃CN; gradient: 90:10 at 0.01 min, 10:90 at 5.0 min, 0:100 at 7.0 min up to 11.0 min, 90:10 at 11.01 min up to 12.0 min; flow rate: 1.0 mL/min; column temperature: RT.

UPLC-MS-11: Waters, YMC Triart C18 particle size: 5 μm; column size: 150×4.6 mm; eluent A: 10 mM ammonium acetate; eluent B: CH₃CN; gradient: 100:0 at 0.01 min, 50:50 at 7.0 min, 0:100 at 9.0 min up to 11.0 min, 100:0 at 11.01 min up to 12.0 min; flow rate: 1.0 mL/min; column temperature: RT.

UPLC-MS-12: Waters, YMC Triart C18; particle size: 5 μm; column size: 150×4.6 mm; eluent A: [water+0.05% TFA]; eluent B: [CH₃CN+0.05% TFA], gradient: 100/0 at 0.01 min, 50/50 at 7 min, 0/100 at 9 min; flow rate: 1.0 mL/min; column temperature: RT.

UPLC-MS-13: X-Bridge C18; particle size: 3.5 μm; 50×4.6 mm; eluent A: 5.0 mM ammonium bicarbonate in water; eluent B: CH₃CN; gradient: 95:5 at 0.01 min, 10:90 at 5.0 min, 5:95 at 5.80 min until 7.20 min, 95:5 at 7.21 min up to 10.0 min; flow rate: 1 mL/min. column temperature: RT.

UPLC-MS-14: Waters, X-bridge C18; particle size: 5.0 μm; column size: 4.6×250 mm; Mobile phase; flow rate: 1 mL/min; Column temperature: RT.

UPLC-MS-15: Waters, X-Bridge C18; particle size: 3.5 um; column size: 50×4.6 mm; eluent: A: 5 mM ammonium bicarbonate in water/B: CH₃CN, gradient: 95/5 at 0.01 min, 15/85 at 2.80 min, 5/95 at 3.50 min the hold for 1.5 min, 95/5 at 5.01 min then hold for 1 min; Flow rate: 1.0 mL/min; Column temperature: RT.

UPLC-MS-16: Waters, X-Bridge C18; particle size: 3.5 μm; column size: 50×4.6 mm; eluent A: 5 mM ammonium bicarbonate in water/B: CH₃CN, gradient: 95/5 at 0.01 min, 10/90 at 3.50 min, 5/95 at 4.50 min hold for 1.5 min, 95/5 at 6.01 min hold for 2 min; Flow rate: 1.0 mL/min; Column temperature: RT.

UPLC-MS-17: Phenomenex, Gemini C6-Phenyl; particule size 3 μm; column size: 100×4.6 mm; eluent: A: [10 mM ammonium bicarbonate+0.1% HCO₂H in Water]; eluent B: MeOH, gradient: 5 to 80% B in 3 min, 80 to 100% in 2 min, 100% for 1 min; flow rate: 1 mL/min, column temperature: RT.

UPLC-MS-18: Waters, X select phenyl hexyl; particle size: 5.0 μm; column size: 4.6×250 mm; Mobile phase; flow rate: 1 mL/min, column temperature: RT.

LCMS-19: Shimadzu LCMS-2020, Kinetex EVO C18; particule size 5 μm; column size: 30×2.1 mm; eluent A: 0.04% TFA in water; eluent B: 0.02% TFA in CH₃CN; gradient: 95:5 at 0.00 min, 5:95 at 0.80 min, 5:95 at 1.20 min, 95:5 at 1.21 min, 95:5 at 1.55 min; flow rate: 1.5 mL/min, column temperature: 50° C.

MS-1: MS flow injection; eluent A: H₂O+4.76% isopropanol+0.05% HCOOH+3.75 mM ammonium acetate; eluent B: isopropanol+0.04% HCOOH; gradient: isocratic 70% B for 0.8 min; flow rate: 0.4 mL/min.

Normal Phase Chromatoqraphy: Normal phase chromatography was run on silica gel using prepacked columns, as detailed below, or using glass columns following standard flash chromatography methodology, unless otherwise stated.
System 1 Teledyne ISCO, CombiFlash Rf
System 2 Biotage Isolera
Column pre-packed RediSep Rf cartridges, or SNAP cartridges
Sample adsorbtion onto Isolute, or on silica gel, or applied as solutions

### Reversed Phase Non-Chiral (RP-HPLC) and Chiral HPLC (C-HPLC):

RP-HPLC-1: Gilson PLC 2020, column: Maisch Reprosil C18 5 μm, 250×30 mm, detection: UV 215 & 254 nM, mobile phase: A: water+0.1% TFA, B: acetonitrile; Gradient: 10 to 95% B in 25 min, flow: 35 mL/min.

RP-HPLC-2: ACCQ prep HP150, column: Waters Xbridge C18 5 μm, 50×100 mm, detection: ELSD and UV 220 nm and 210-450 nm, mobile phase, flow: 100 mL/min).

RP-HPLC-3: Waters, column: X-bridge C18 OBD 5 μm, 100×30 mm, detection UV, mobile phase; flow rate: 40 mL/min.

RP-HPLC-4: Gilson, column: SunFire C18 OBD 5 μM; 100×30 mm; detection UV 254 nM, mobile phase; flow rate: 40 mL/min; column temperature: 25° C.

RP-HPLC-5: Shimadzu LC-20AP; column: X-bridge C18 5 μm; 250×19 mm; detection UV 202 & 220 nM, mobile phase; flow rate: 15 mL/min; column temperature: 40° C.

RP-HPLC-6: Shimadzu LC-20AP; column: X-bridge C18 5 μm; 250×19 mm; detection UV 202 & 220 nM, mobile phase; flow rate: 13 mL/min; column temperature: 40° C.

RP-HPLC-7: Waters HPLC e2695; column: Waters X-Bridge C8 5 μm; 250×4.6 mm; detection: PDA; mobile phase; flow rate: 1 mL/min; column temperature: 25° C. RP-HPLC-8

RP-HPLC-8: Waters HPLC e2695; column: Waters X-Bridge C18 5 μm; 150×4.6 mm; detection: UV; mobile phase: (A: [water+0.1% NH₃]/B: [CH₃CN+0.1% NH₃], gradient: 10 to 90% B in 5 min, 90 to 95% in 6 min with a plateau at 95% for 4 min); flow rate: 1 mL/min; column temperature: 25° C.

RP-HPLC-9: Waters HPLC e2695; column: Waters XBridge C18, 5 μm; 150×4.6 mm; detection: PDA; mobile phase; flow rate: 1.0 mL/min; column temperature: 25° C.

RP-HPLC-10: Agilent HPLC 1260 infinity series; column: YMC Triart C18, 5 μm; 150×4.6 mm; detection: PDA; mobile phase: (A: [water+0.1% HCOOH]/B: [CH₃CN+0.1% HCOOH], gradient: 10 to 90% B in 5 min, 90 to 95% in 6 min with a plateau at 95% for 4 min); flow rate: 1.0 mL/min; column temperature: 25° C.

RP-HPLC-11: Waters, X select phenyl hexyl; particle size: 5.0 μm; column size: 4.6×250 mm, PDA; mobile phase; flow rate; column temperature: RT.

C-HPLC-1: column: Chiralpak IA 5 μm; 250×30 mm, detection UV 240 nM, mobile phase, flow rate: 20 mL/min. column temperature: RT.

C-HPLC-2: column: Chiralpak IA 5 μm, 250×4.6 mm, detection UV 220/254 nM, mobile phase, flow rate: 1 mL/min, column temperature: 25° C.

C-HPLC-3: column: Chiralpak IA 5 μm; 250×30 mm, detection UV 240 nM, mobile phase, flow rate: 15 mL/min. column temperature: RT.

C-HPLC-4: column: Chiralpak IC 5 μm; 250×20 mm; detection UV 270 nM, mobile phase; flow rate: 10 mL/min; column temperature: 25° C.

C-HPLC-5: column: Chiralpak IC-3 3 μm; 100×3 mm; detection UV 270 nM, mobile phase; flow rate: 0.42 mL/min; column temperature: 25° C.

C-HPLC-6: column: Chiralpak IC 5 μm; 250×4.6 mm; detection UV 240 nM, mobile phase; flow rate: 1 mL/min; column temperature: 25° C.

C-HPLC-7: column: Chiralpak IC 5 μm; 250×30 mm; detection UV 270 nM, mobile phase; flow rate: 20 mL/min; column temperature: 25° C.

C-HPLC-8: column: Chiralpak AD 5 μm; 250×30 mm; detection UV 230 nM, mobile phase; flow rate: 20 mL/min; column temperature: 25° C.

C-HPLC-9: column: Chiralpak AD-3 3 μm; 100×3 mm; detection UV 130 or 250 nM, mobile phase; flow rate: 0.42 mL/min; column temperature: 25° C.

C-HPLC-10: column: Chiralpak AD 5 μm; 250×25 mm; detection UV 230 nM, mobile phase; flow rate: 15 mL/min; column temperature: 25° C.

C-HPLC-11: column: Chiralpak IG-3 3 μm; 100×3 mm; mobile phase; flow rate: 0.42 mL/min; column temperature: 25° C.

C-HPLC-12: column: Chiralpak IG 5 μm; 250×20 mm; mobile phase; flow rate: 10 mL/min; column temperature: 25° C.

C-HPLC-13: column: Chiralpak ID 5 μm; 250×25 mm; detection UV 230 nM, mobile phase; flow rate: 15 mL/min; column temperature: RT.

C-HPLC-14: column: Chiralpak ID 5 μm; 250×30 mm; detection UV 230 nM, mobile phase; flow rate: 30 mL/min; column temperature: 30° C.

C-HPLC-15: column: Chiralpak ID 5 μm; 250×25 mm; detection UV 254 nM, mobile phase; flow rate: 10 mL/min; column temperature: RT.

C-HPLC-16: column: Chiralpak ID 5 μm; 250×4.6 mm; detection UV 254 nM, mobile phase; flow rate: 1 mL/min; column temperature: RT.

C-HPLC-17: column: Lux C2 5 μm; 250×30 mm; detection UV 210 nM, mobile phase; flow rate: 42 mL/min; column temperature: 25° C.

C-HPLC-18: column: Lux C2 5 μm; 150×4.6 mm; detection UV 254 nM, mobile phase; flow rate: 1 mL/min; column temperature: 25° C.

C-HPLC-19: column: Chiralpak AS 5 μm; 250×20 mm; detection UV 240 nM, mobile phase; flow rate: 10 mL/min; column temperature: 25° C.

C-HPLC-20: column: Chiralpak AS 3 μm; 100×3 mm; detection UV 240 nM, mobile phase; flow rate: 0.42 mL/min; column temperature: 25° C.

C-HPLC-21: column: Chiralcel OZ 5 μm; 250×20 mm; detection UV 280 nM, mobile phase; flow rate: 10 mL/min; column temperature: RT.

C-HPLC-22: column: Chiralcel OZ-3 3 μm; 100×3 mm; detection UV 254 nM, mobile phase; flow rate: 0.42 mL/min; column temperature: 25° C.

C-HPLC-23: Shimadzu LC-20AP; ChiralpakAD-H, 5 μm; 250×21 mm; detection: UV; mobile phase; flow rate: 20 mL/min; column temperature: 40° C.

C-HPLC-24: Agilent 1260 infinity HPLC; column: Chiralpak AD-H 5 μm; 250×4.6 mm; detection: PDA; mobile phase; flow rate: 1 mL/min; column temperature: 25° C.

C-HPLC-25: Shimadzu LC-20AP; column CHIRACEL OX-H 5 μm; 250×21 mm; detection UV 202 & 220 nM; mobile phase; flow rate; column temperature: 40° C.

C-HPLC-26: Agilent 1260 infinity; column Chiralpak OX-H 5 μm; 250×4.6 mm; detection: PDA; mobile phase; flow rate: 1 mL/min; column temperature: 25° C.

C-HPLC-27: Shimadzu LC-20AP; column Chiralpak IG 5 μm; 250×21 mm; detection UV 202 & 220 nM, mobile phase; flow rate: 22 mL/min; column temperature: 40° C.

C-HPLC-28: Shimadzu LC-20AP with UV detector; column: Chiralpak IG 5 μm; 250×21 mm; mobile phase; flow rate: 12 mL/min; column temperature: 40° C.

C-HPLC-29: Agilent 1260 infinity HPLC with PDA detector; column: Chiralpak IG 5 μm; 250×4.6 mm; mobile phase; flow rate: 1 mL/min; column temperature: 25° C.

C-HPLC-30: Shimadzu LC-20AP; column Chiralpak IBN 5 μm; 250×21 mm; detection; UV, mobile phase; flow rate: 22 mL/min; column temperature: 40° C.

C-HPLC-31: Agilent 1260 infinity HPLC; column Chiralpak IBN 5 μm; 250×4.6 mm; detection: PDA; mobile phase; flow rate: 1 mL/min; column temperature: 25° C.

C-HPLC-32: Shimadzu LC-20AP; column: Chiralpak IC 5 μm; 250×21 mm; detection: PDA; mobile phase; flow rate: 1 mL/min; column temperature: 40° C.

C-HPLC-33: Agilent 1260 infinity HPLC; column: Chiralpak IC 5 μm; 250×4.6 mm; detection: PDA; mobile phase; flow rate: 1 mL/min; column temperature: 25° C.

C-HPLC-34: Shimadzu LC-20AP; column: Chiralpak IH 5 μm; 250×21 mm; detection: UV; mobile phase; flow rate 20 mL/min; column temperature: 40° C.

C-HPLC-35: column: Lux Cellulose-2 5 μm; 250×20 mm; detection: UV; mobile phase; flow rate: 5.5 mL/min; column temperature: RT.

### Chiral SFC Methods (C-SFC):

C-SFC-1: column: Amylose-C NEO 5 μm; 250×30 mm; mobile phase; flow rate: 80 mL/min; column temperature: 40° C.; back pressure: 120 bars.

C-SFC-2: column: Lux Amylose-1 5 μm; 250×30 mm; mobile phase; flow rate: 80 mL/min; column temperature: 40° C.; back pressure: 120 bars.

C-SFC-3: column: Chiralpak AD-H 5 μm; 100×4.6 mm; mobile phase; flow rate: 3 mL/min; column temperature: 40° C.; back pressure: 1800 psi.

C-SFC-4: column: Chiralpak AD-H 5 μm; 250×30 mm; mobile phase; flow rate: 80 mL/min; column temperature: 40° C.; back pressure: 120 bars.

C-SFC-5: column: Chiralpak IB 5 μm; 250×30 mm; mobile phase; flow rate: 80 mL/min; column temperature: 40° C.; back pressure: 120 bars.

C-SFC-6: column: Chiralpak IB 5 μm; 100×4.6 mm; mobile phase; flow rate: 3 mL/min; column temperature: 40° C.; back pressure: 1800 psi.

C-SFC-7: column: Chiralpak IG 5 μm; 250×30 mm; mobile phase; flow rate: 80 mL/min; column temperature: 40° C.; back pressure: 120 bars.

C-SFC-8: column: Chiralpak IG 5 μm; 100×4.6 mm; mobile phase; flow rate: 3 mL/min; column temperature: 40° C.; back pressure: 1800 psi.

C-SFC-9: column: Chiralpak IC 5 μm; 250×30 mm; mobile phase; flow rate: 60 mL/min; column temperature: 40° C.; back pressure: 105 bars.

C-SFC-10: column: Chiralpak IC 5 μm; 100×4.6 mm; mobile phase; flow rate: 3 mL/min; column temperature: 40° C.; back pressure: 1800 psi.

C-SFC-11: column: Lux Amylose-1 5 μm; 100×4.6 mm; mobile phase; flow rate: 3 mL/min; column temperature: 40° C.; back pressure: 1800 psi.

C-SFC-12: column: Chiralpak AY 10 μm; 300×50 mm; mobile phase; flow rate: 200 mL/min; column temperature: 38° C.; back pressure: 100 bars.

C-SFC-13: column: (S,S) Whelk O1 10 μm; 300×50 mm I.D.; mobile phase; flow rate: 200 mL/min; column temperature: 38° C.; back pressure: 100 bars.

C-SFC-14: column: Chiralpak IG 3 μm; 100×4.6 mm I.D.; mobile phase; flow rate: 2.5 mL/min; column temperature: 35° C.; back pressure: 100 bars.

C-SFC-15: column: Lux i-Cellulose-5 5 μm; 250×30 mm; mobile phase; flow rate: 80 mL/min; column temperature: 40° C.; back pressure: 120 bars.

C-SFC-16: column: Chiralpak IG 5 μm; 250×30 mm; mobile phase; flow rate: 70 mL/min; column temperature: 40° C.; back pressure: 120 bars.

C-SFC-17: column: (S,S) Whelk O1 5 μm; 250×4.6 mm I.D.; mobile phase; flow rate: 2.5 mL/min; column temperature: 35° C.; back pressure: 100 bars.

C-SFC-18: column: Lux Cellulose-2 5 μm; 250×30 mm; mobile phase; flow rate: 80 mL/min; column temperature: 40° C.; back pressure: 120 bars.

C-SFC-19: column: Lux Cellulose-2 (OZ) 5 μm; 100×4.6 mm; mobile phase; flow rate: 3 mL/min; column temperature: 40° C.; back pressure: 1800 psi.

C-SFC-20: column: Lux Amylose-1 5 μm; 250×30 mm; mobile phase; flow rate: 90 mL/min; column temperature: 40° C.; back pressure: 105 bars.

C-SFC-21: column: Chiralpak IG 10 μm; 300×50 mm; mobile phase; flow rate: 200 mL/min; column temperature: 38° C.; back pressure: 100 bars.

C-SFC-22: column: Chiralpak IG 3 μm; 100×4.6 mm; mobile phase; flow rate: 2.5 mL/min; column temperature: 35° C.; back pressure: 100 bars.

C-SFC-23: column: Chiralpak AD-H 5 μm; 250×30 mm; mobile phase; flow rate: 90 mL/min; column temperature: 40° C.; back pressure: 140 bars.

C-SFC-24: column: Chiralpak IB 5 μm; 250×30 mm; mobile phase; flow rate: 75 mL/min; column temperature: 40° C.; back pressure: 100 bars.

C-SFC-25: column: Chiralpak AD-H 5 μm; 250×30 mm; mobile phase; flow rate: 80 mL/min; column temperature: 40° C.; back pressure: 130 bars.

C-SFC-26: column: Chiralpak IG 5 μm; 230×30 mm; mobile phase; flow rate: 80 mL/min; column temperature: 40° C.; back pressure: 120 bars.

C-SFC-27: column: Chiralpak IB-N 5 μm; 250×30 mm; mobile phase; flow rate: 90 mL/min; column temperature: 40° C.; back pressure: 120 bars.

C-SFC-28: column: Chiralpak Chiralcel OD 10 μm; 250×30 mm; mobile phase; flow rate: 70 mL/min; column temperature: 38° C.; back pressure: 100 bars.

C-SFC-29: column: Chiralcel OD-3 3 μm; 150×4.6 mm; mobile phase; flow rate: 2.5 mL/min; column temperature: 35° C.; back pressure: 1500 psi.

C-SFC-30: column: Chiralpak AD-H 5 μm; 250×30 mm; mobile phase; flow rate: 90 mL/min; column temperature: 40° C.; back pressure: 105 bars.

C-SFC-31: column: Chiralpak OX 5 μm; 100×4.6 mm; mobile phase; flow rate: 4 mL/min; column temperature: 40° C.; back pressure: 2500 psi.

C-SFC-32: column: Lux Cellulose-2 5 μm; 250×30 mm; mobile phase; flow rate: 90 mL/min; column temperature: 40° C.; back pressure: 105 bars.

C-SFC-33: column: Chiralpak AD-H 5 μm; 250×30 mm; mobile phase; flow rate: 80 mL/min; column temperature: 40° C.; back pressure: 140 bars.

C-SFC-34: column: Lux Amylose-1 5 μm; 250×30 mm; mobile phase; flow rate: 90 mL/min; column temperature: 40° C.; back pressure: 120 bars.

C-SFC-35: column: Chiralpak AD-H 5 μm; 250×30 mm; mobile phase; flow rate: 80 mL/min; column temperature: 40° C.; back pressure: 110 bars.

C-SFC-36: column: Lux Cellulose-2 5 μm; 250×30 mm; mobile phase; flow rate: 100 mL/min; column temperature: 40° C.; back pressure: 105 bars.

C-SFC-37: column: Lux Cellulose-2 5 μm; 100×4.6 mm; mobile phase; flow rate: 3 mL/min; column temperature: 40° C.; back pressure: 124 bars.

C-SFC-38: column: Chiralpak AS 10 μm; 250×30 mm; mobile phase; flow rate: 80 mL/min; column temperature: 38° C.; back pressure: 100 bars.

C-SFC-39: column: Chiralpak AS 3 μm; 150×4.6 mm; mobile phase; flow rate: 2.5 mL/min; column temperature: 35° C.; back pressure: 100 bars.

C-SFC-40: column: Lux Cellulose-2 3 μm; 150×4.6 mm; mobile phase; flow rate: 2.5 mL/min; column temperature: 35° C.; back pressure: 100 bars.

C-SFC-41: column: Chiralpak IG 10 μm; 250×50 mm; mobile phase; flow rate: 250 mL/min; column temperature: 35° C.; back pressure: 100 bars.

C-SFC-42: column: Chiralpak IG-3 3 μm; 50×4.6 mm; mobile phase; flow rate: 3 mL/min; column temperature: 35° C.; back pressure: 100 bars.

C-SFC-43: column: Chiralpak IG 10 μm; 250×30 mm; mobile phase; flow rate: 200 mL/min; column temperature: 35° C.; back pressure: 100 bars.

C-SFC-44: column: Lux Cellulose-2 5 μm; 250×30 mm; mobile phase; flow rate: 80 mL/min; column temperature: 40° C.; back pressure: 110 bars.

C-SFC-45: column: Chiralpak AY 5 μm; 250×30 mm; mobile phase; flow rate: 50 mL/min; column temperature: 38° C.; back pressure: 100 bars.

C-SFC-46: column: Chiralcel OZ 5 μm; 100×4.6 mm; mobile phase; flow rate: 3 mL/min; column temperature: 40° C.; back pressure: 100 bars.

C-SFC-47: Waters PSFC-200; column: CHIRALCEL OX-H 5 μm; 250×21 mm; detection UV, mobile phase; flow rate: 80 mL/min; column temperature: 40° C.; back pressure: 100 bars.

C-SFC-48: Waters SFC investigator; column: Chiralcel OX-H; 5 μm; 250×4.6 mm; detection: PDAmobile phase; flow rate: 4 mL/min; column temperature: 40° C.; back pressure: 100 bars.

C-SFC-49: Waters SFC investigator with PDA detector; column: Chiralpak AD-H 5 μm; 250×4.6 mm; mobile phase; flow rate: 4 mL/min; column temperature: 40° C.; back pressure: 100 bars.

C-SFC-50: Waters SFC 200 with UV detector; column: Chiralpak IG; 5 μm; 250×21 mm; mobile phase; flow rate: 80 mL/min; column temperature: 40° C.; back pressure: 100 bars.

C-SFC-51: Waters SFC investigator with PDA detector; column: Chiralpak IG 5 μm; 250×4.6 mm; mobile phase; flow rate: 4 mL/min; column temperature: 40° C.; back pressure: 100 bars.

C-SFC-52: Waters SFC 200 with UV detector; column: Chiralpak IC 5 μm; 250×21 mm; mobile phase; flow rate: 80 mL/min; column temperature: 40° C.; back pressure: 100 bars.

C-SFC-53: Waters SFC investigator with PDA detector; column: Chiralpak IC 5 μm; 250×4.6 mm; mobile phase; flow rate: 4 mL/min; column temperature: 40° C.; back pressure: 100 bars.

C-SFC-54: Waters SFC 200 with UV detector; column: Chiralpak IB-N 5 μm; 250×21 mm; mobile phase; flow rate: 80 mL/min; column temperature: 40° C.; back pressure: 100 bars.

C-SFC-55: Waters SFC investigator with PDA detector; column: Chiralpak IB-N 5 μm; 250×4.6 mm; mobile phase; flow rate: 4 mL/min; column temperature: 40° C.; back pressure: 100 bars.

### Abbreviations:

Abbreviations used are those conventional in the art.

| Abbreviation | Describtion |
|---|---|
| Ac₂O | acetic anhydride |
| AcOH | acetic acid |
| AIBN | 2,2′-azobis(2-methylpropionitrile) |
| aq. | aqueous |
| Ar | argon |
| B₂Pin₂ | 4,4,4′,4′,5,5,5′,5′-Octamethyl-2,2′-bi(1,3,2-dioxaborolane) |
| BPR | back pressure |
| brine | saturated aqueous sodium chloride |
| n-BuLi | n-butyllithium |
| CataCXium- | (Di(1-adamantyl)-butylphosphine)-2-(2′-amino-1,1′- |
| A-Pd-G3 | biphenyl)palladium(II) methanesulfonate |
| CBz- | N-(Benzyloxycarbonyloxy)succinimide |
| succinimide | |
| CDI | carbonyldiimidazole |
| conc. | concentrated |
| CPME | Cyclopentylmethyl ether |
| DAST | N,N-diethyl-1,1,1-trifluoro-λ⁴-sulfanamine |
| DCC | dicyclohexylmethanediimine |
| DCE | dichloroethane |
| DEA | diethylamine |
| DEAD | diethyl azodicarboxylate |
| DHP | 3,4-dihydropyran |
| DIPEA | N,N-diisopropylethylamine, N-ethyl-N- |
| | isopropylpropan-2-amine |
| DMA | N,N-dimethylacetamide |
| DMAP | N,N-dimethylpyridin-4-amine |
| DME | Dimethoxyethane |
| DMF | N,N-Dimethylformamide |
| DMF-DMA | 1,1-dimethoxy-N,N-dimethylmethanamine |
| DMPU | 1,3-dimethyl-3,4,5,6-tetrahydro-2(1H)-pyrimidinone |
| DMSO | dimethylsulfoxide |
| DMSO-d₆ | hexadeuterodimethyl sulfoxide |
| dppf | 1,1′-bis(diphenylphosphanyl) ferrocene |
| EDC•HCl | 1-ethyl-3-carbodiimide hydrochloride |
| ee | enantiomeric excess |
| ESI-MS | electrospray ionization mass spectroscopy |
| EtOAc | ethyl acetate |
| EtOH | ethanol |
| GBq | gigabecquerel |
| h | Hour (s) |
| HATU | 1-Bis(dimethylamino)methylene-1H-1,2,3-triazolo4,5- |
| | bpyridinium 3-oxid hexafluorophosphate |
| HPLC | high-performance liquid chromatography |
| HOBT | 1-hydroxybenzotriazole |
| IPA | 2-propanol |
| KOAc | potassium acetate |
| KOH | potassium hydroxide |
| Lawesson | 2,4-bis(4-methoxyphenyl)-2,4-dithioxo-1,3,2,4- |
| | dithiadiphosphetane |
| L/mL/□L | litre/millilitre/microlitre |
| LC-MS | liquid chromatography and mass spectroscopy |
| M, N | molar |
| MeMgBr | methylmagnesium bromide |
| MeOH | methanol |
| min | minutes |
| MOM-Cl | chloromethyl methyl ether |
| MP-carbonate | Tetraalkylammonium carbonate, polymer-bound |
| resin | |
| MTBE | methyl tert-butyl ether |
| MW, mw | microwave |
| m/z | mass to charge ratio |
| N₂ | nitrogen |
| NaOtBu | sodium tert-butoxide |
| NBS | N-bromosuccinimide |
| NCS | N-chlorosuccinimide |
| NIS | N-iodosuccinimide |
| NEt₃, Et₃N, | triethylamine |
| TEA | |
| NMO | N-methyl morpholine oxide |
| NMR | nuclear magnetic resonance |
| Pd(PPh₃)₄ | tetrakis(triphenylphosphane)palladium(0) |
| iPrMgCl | Isopropylmagnesium chloride |
| PTSA or pTSA | p-toluenesulfonic acid |
| RM | reaction mixture |
| RP | reversed phase |
| Rt | retention time |
| RT | room temperature |
| RuPhos | 2-dicyclohexylphosphino-2′,6′-diisopropoxybiphenyl |
| RuPhos-Pd-G3 | (2-dicyclohexylphosphino-2′,6′-diisopropoxy-1,1′- |
| | biphenyl)[2-(2′-amino-1,1′-biphenyl)]palladium(II) |
| | methanesulfonate |
| Sat., sat. | saturated |
| SFC | supercritical fluid chromatography |
| TBAF | Tetrabutylammonium fluoride |
| TBDPS- | tert-butyl(chloro)diphenylsilane |
| chloride | |
| tBME, TBME, | tert-butyl methyl ether |
| TBMe | |
| TBq | terabecquerel |
| t-BuOH | tert-butanol |
| tBuXPhos- | tBuXPhos-Pd-G3, [(2-Di-tert-butylphosphino-2′,4′,6′- |
| Pd-G3 | triisopropyl-1,1′-biphenyl)-2-(2′-amino-1,1′-biphenyl)] |
| | palladium(II) methanesulfonate |
| TFA | trifluoroacetic acid |
| TFAA | 2,2,2-trifluoroacetic anhydride |
| THF | tetrahydrofuran |
| TLC | thin-layer chromatography |
| TMS-Chloride | Trimethylsilyl chloride |
| TMSI | trimethylsulfoxonium iodide |
| T₃P | propylphosphonic anhydride |
| TPAP | tetrapropylammonium perruthenate |
| TsCl | tosyl chloride, 4-Methylbenzene-1-sulfonyl chloride |
| UPLC | ultra-performance liquid chromatography |
| XPhos | 2-dicyclohexylphosphino-2′,4′,6′-triisopropylbiphenyl |
| XPhos-Pd-G3 | (2-dicyclohexylphosphino-2′,4′,6′-triisopropyl-1,1′- |
| | biphenyl)[2-(2′-amino-1,1′-biphenyl)]palladium(II) |
| | methanesulfonate |

The asterisk * indicated in the name of a compound indicates relative stereochemistry e.g. (3aS*,7aS*)-5-(1-(2-acryloyl-2-azaspiro[3.3]heptan-6-yl)-4-(5-chloro-6-methyl-1H-indazol-4-yl)-5-methyl-1H-pyrazol-3-yl)-2-methyloctahydro-1H-pyrrolo[3,4-c]pyridin-1-one indicates either (3a5,7a5)-5-(1-(2-acryloyl-2-azaspiro[3.3]heptan-6-yl)-4-(5-chloro-6-methyl-1H-indazol-4-yl)-5-methyl-1H-pyrazol-3-yl)-2-methyloctahydro-1H-pyrrolo[3,4-c]pyridin-1-one or (3aR,7aR)-5-(1-(2-acryloyl-2-azaspiro[3.3]heptan-6-yl)-4-(5-chloro-6-methyl-1H-indazol-4-yl)-5-methyl-1H-pyrazol-3-yl)-2-methyloctahydro-1H-pyrrolo[3,4-c]pyridin-1-one.

### Preparation of Final Compounds

### Method-1 for the Preparation of Example 1a: 1-(6-(3-(4-Acetyl-2,2-dimethylpiperazin-1-yl)-4-(5,6-dimethyl-1H-indazol-4-yl)-5-methyl-1H-pyrazol-1-yl)-2-azaspiro[3.3]heptan-2-yl)prop-2-en-1-one

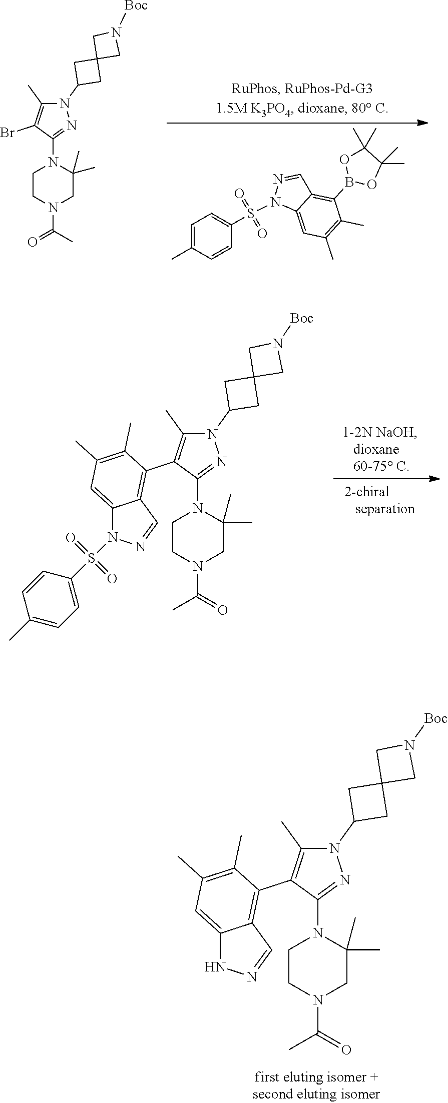

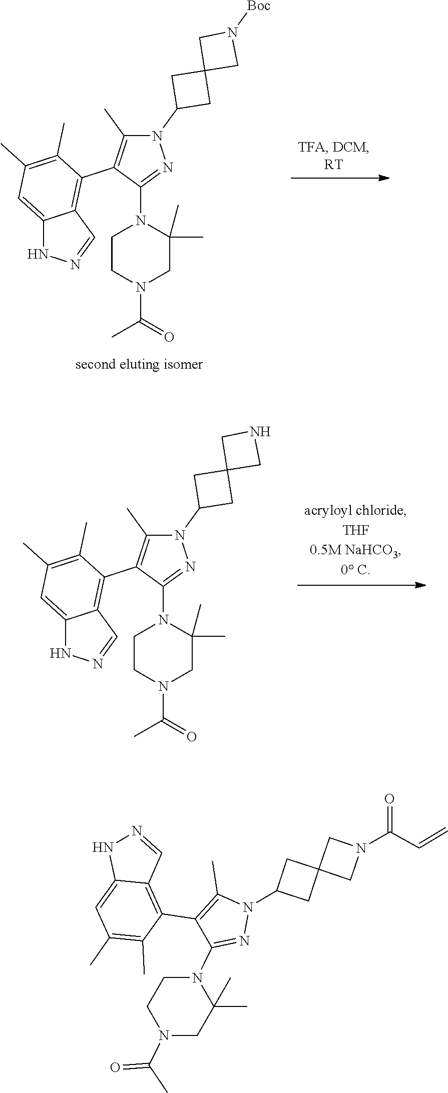 [SMILES: CC(=O)N1CCN(c2nn(C3CC4(C3)CN(C(=O)OC(C)(C)C)C4)c(C)c2-c2c(C)c(C)cc3[nH]ncc23)C(C)(C)C1 ; CC(=O)N1CCN(c2nn(C3CC4(CNC4)C3)c(C)c2-c2c(C)c(C)cc3[nH]ncc23)C(C)(C)C1 ; C=CC(=O)N1CC2(CC(n3nc(N4CCN(C(C)=O)CC4(C)C)c(-c4c(C)c(C)cc5[nH]ncc45)c3C)C2)C1]

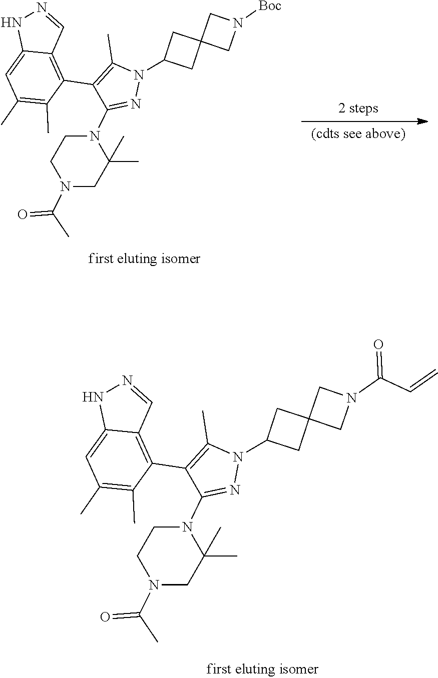 [SMILES: CC(=O)N1CCN(c2nn(C3CC4(C3)CN(C(=O)OC(C)(C)C)C4)c(C)c2-c2c(C)c(C)cc3[nH]ncc23)C(C)(C)C1 ; C=CC(=O)N1CC2(CC(n3nc(N4CCN(C(C)=O)CC4(C)C)c(-c4c(C)c(C)cc5[nH]ncc45)c3C)C2)C1]

### Step 1: Tert-butyl 6-(3-(4-acetyl-2,2-dimethylpiperazin-1-yl)-4-(5,6-dimethyl-1-tosyl-1H-indazol-4-yl)-5-methyl-1H-pyrazol-1-yl)-2-azaspiro[3.3]heptane-2-carboxylate

A mixture of tert-butyl 6-(3-(4-acetyl-2,2-dimethylpiperazin-1-yl)-4-bromo-5-methyl-1H-pyrazol-1-yl)-2-azaspiro[3.3]heptane-2-carboxylate (Intermediate C10, 2.47 g, 4.84 mmol), 5,6-dimethyl-4-(4,4,5,5-tetramethyl-1,3,2-dioxaborolan-2-yl)-1-tosyl-1H-indazole (Intermediate D5, 2.48 g, 5.81 mmol), RuPhos (226 mg, 0.48 mmol) and RuPhos-Pd-G3 (405 mg, 0.48 mmol) in dioxane (50 mL) was treated with K₃PO₄ (2M aq., 9.68 mL, 14.5 mmol) and the mixture was stirred at 80° C. for 1 h. The reaction mixture was poured into a sat. aq. NaHCO₃ solution and extracted with EtOAc (3×). The combined organic layers were dried (MgSO₄) and concentrated. The crude residue was diluted with THF (100 mL), SiliaMetS®Thiol (1.94 mmol) was added and the mixture swirled for 1 h at 40° C. The mixture was filtered, washed through with THF and the filtrate was concentrated. The crude residue was purified by normal phase chromatography (eluent: 8/2 MTBE/iPrOH in heptane 0 to 53%) to give the title compound as a beige foam. UPLC-MS-1a: Rt=1.41 min; MS m/z [M+H]+ 730.6.

### Step 2: Tert-butyl 6-(3-(4-acetyl-2,2-dimethylpiperazin-1-yl)-4-(5,6-dimethyl-1H-indazol-4-yl)-5-methyl-1H-pyrazol-1-yl)-2-azaspiro[3.3]heptane-2-carboxylate

A mixture of tert-butyl 6-(3-(4-acetyl-2,2-dimethylpiperazin-1-yl)-4-(5,6-dimethyl-1-tosyl-1H-indazol-4-yl)-5-methyl-1H-pyrazol-1-yl)-2-azaspiro[3.3]heptane-2-carboxylate (Step 1, 2.59 g, 3.55 mmol), NaOH (2N aq., 8.87 mL, 17.7 mmol) and dioxane (35 mL) was stirred at 60° C. for 4 h and then at 75° C. for 3 h. The reaction mixture was cooled to RT, diluted with water and extracted with EtOAc (2×). The combined organic layers were washed with brine, dried (Na₂SO₄) and concentrated. The isomers were separated by chiral SFC (C-SFC-4; mobile phase: 35:65 IPA/CO₂) to give the title compound as the second eluting peak: UPLC-MS-1a: Rt=1.18 min; MS m/z [M+H]⁺ 576.5; C-SFC-3 (mobile phase: 35:65 IPA/CO₂): Rt=2.58 min. The other isomer was obtained as the first eluting peak: C-SFC-3 (mobile phase: 35:65 IPA/CO₂): Rt=1.01 min.

### Step 3: 1-(4-(4-(5,6-Dimethyl-1H-indazol-4-yl)-5-methyl-1-(2-azaspiro[3.3]heptan-6-yl)-1H-pyrazol-3-yl)-3,3-dimethylpiperazin-1-yl)ethan-1-one

A solution of tert-butyl 6-(3-(4-acetyl-2,2-dimethylpiperazin-1-yl)-4-(5,6-dimethyl-1H-indazol-4-yl)-5-methyl-1H-pyrazol-1-yl)-2-azaspiro[3.3]heptane-2-carboxylate (Step 2 second eluting isomer, 1.14 g, 1.96 mmol) in CH₂Cl₂ (20 mL) was treated with TFA (3.78 mL, 49.0 mmol) and the mixture was stirred at RT for 2 h. The reaction mixture was poured into a mixture of sat. aq. NaHCO₃ and EtOAc, stirred for 15 min, saturated with NaCl and then the mixture was extracted with EtOAc (4×). The combined organic layers were dried (Na₂SO₄) and concentrated to give the title compound as a yellow foam which was used without purification in the next step. UPLC-MS-1a: Rt=0.68 min; MS m/z [M+H]⁺ 476.4.

### Step 4: 1-(6-(3-(4-Acetyl-2,2-dimethylpiperazin-1-yl)-4-(5,6-dimethyl-1H-indazol-4-yl)-5-methyl-1H-pyrazol-1-yl)-2-azaspiro[3.3]heptan-2-yl)prop-2-en-1-one

A solution of 1-(4-(4-(5,6-dimethyl-1H-indazol-4-yl)-5-methyl-1-(2-azaspiro[3.3]heptan-6-yl)-1H-pyrazol-3-yl)-3,3-dimethylpiperazin-1-yl)ethan-1-one (Step 3, 1.96 mmol) in THF (59 mL) was treated with NaHCO₃ (0.5M aq., 11.8 mL, 5.90 mmol) and the mixture was cooled to 0° C. Acryloyl chloride (0.17 mL, 2.06 mmol) was added in 4 portions within 2 min, followed by additional acryloyl chloride (0.03 mL, 0.39 mmol) 15 min later. After 30 min, the reaction mixture was diluted with 1M aq. NaHCO₃ and extracted with EtOAc (2×). The combined organic phases were washed with brine, dried (Na₂SO₄) and concentrated. The residue was dissolved in MeOH (20 mL), stirred at 40° C. for 10 min until disappearance (UPLC) of the side product resulting from reaction of the acryloyl chloride with the indazole NH. The mixture was concentrated and the residue was diluted with 1:1 water/brine and extracted with EtOAc (2×). The combined organic layers were washed with brine, dried (Na₂SO₄) and concentrated. The crude residue was purified by normal phase chromatography (eluent: CH₂Cl₂/MeOH/Et₃N/(200:20/2) in CH₂Cl₂ from 0 to 100%) to give after lyophilization (from t-BuOH) Example 1a as a white solid: ¹H NMR (400 MHz, DMSO-d₆) δ 12.71 (s, 1H), 7.47 (s, 1H), 7.27 (s, 1H), 6.29 (m, 1H), 6.09 (m, 1H), 5.67 (m, 1H), 4.73 (m, 1H), 4.34 (s, 1H), 4.28 (s, 1H), 4.05 (s, 1H), 3.99 (s, 1H), 2.96-3.21 (m, 4H), 2.60-2.83 (m, 6H), 2.38 (s, 3H), 2.10 (s, 3H), 1.92 (s, 6H), 1.00-1.12 (m, 6H); UPLC-MS-1a: Rt=0.93 min; MS m/z [M+H]⁺ 530.4; C-SFC-3 (mobile phase 45:55 (IPA+0.1% NH₃)/CO₂): Rt=1.85 min.

Example 1b: was prepared by a similar method using tert-butyl 6-(3-(4-acetyl-2,2-dimethylpiperazin-1-yl)-4-(5,6-dimethyl-1H-indazol-4-yl)-5-methyl-1H-pyrazol-1-yl)-2-azaspiro[3.3]heptane-2-carboxylate (Step 2 first eluting isomer): C-SFC-3 (mobile phase 45:55 (IPA+0.1% NH₃)/CO₂): Rt=0.72 min.

Method-1a: similar to Method-1 except that the 2 isomers were separated after the last step (Step 4).

The following examples 2a to 5 were prepared using analogous methods to Method-1 from intermediates (in Step 1) described in the intermediates synthesis section or commercially available.

| | | Method, intermediates | |
|---|---|---|---|
| | | and chiral separation | |
| | | conditions used for the | |
| | | separation of the | |
| | | enantiomers Step 2 | |
| | | (Method-1) or Step 4 | |
| | | (Method-1a) and order of | |
| Example | Structure | elution | Characterizing data |
| 2a/2b | 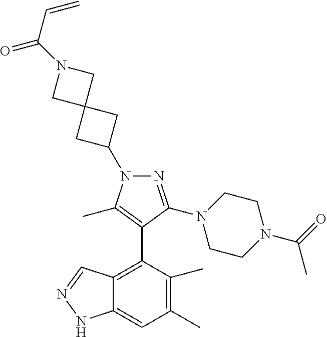 [SMILES: C=CC(=O)N1CC2(CC(n3nc(N4CCN(C(C)=O)CC4)c(-c4c(C)c(C)cc5[nH]ncc45)c3C)C2)C1] | Using Method-1a from Intermediate C12 (Step 1) and D5 and C-HPLC-10 (mobile phase: n- heptane/EtOH 70/30); Example 2a = 1^{st} eluting isomer, Example 2b = 2^{nd} eluting isomer | Example 2b: ¹H NMR (400 MHz, DMSO-d₆) δ 12.77 (s, 1H), 7.52 (s, 1H), 7.29 (s, 1H), 6.39-6.26 (m, 1H), 6.14-6.05 (m, 1H), 5.70- 5.63 (m, 1H), 4.72-4.66 (m, 1H), 4.34 (s, 1H), 4.28 (s, 1H), 4.05 (s, 1H), 3.99 (s, 1H), 3.28-3.15 (m, 3H), 2.86-2.72 (m, 5H), 2.72- 2.65 (m, 4H), 2.39 (s, 3H), 2.10 (s, 3H), 1.89 (s, 3H), 1.87 (s, 3H); UPLC-MS-1d: Rt = 3.96 min; MS m/z [M + H]⁺ 502.5; C-HPLC-9 (mobile phase: heptane/EtOH 70/30): Rt = 6.18 min, Example 2a: C- HPLC-9 (mobile phase: heptane/EtOH 70/30): Rt = 3.30 min. |
| | 1-(6-(3-(4- | | |
| | acetylpiperazin-1-yl)-4- | | |
| | (5,6-dimethyl-1H- | | |
| | indazol-4-yl)-5-methyl- | | |
| | 1H-pyrazol-1-yl)-2- | | |
| | azaspiro[3.3]heptan-2- | | |
| | yl)prop-2-en-1-one | | |
| 3a/3b | 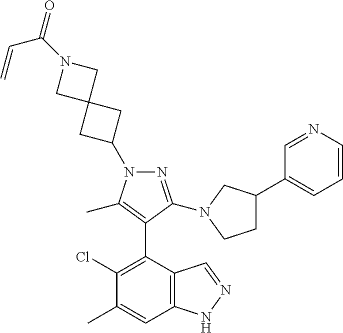 [SMILES: C=CC(=O)N1CC2(CC(n3nc(N4CCC(c5cccnc5)C4)c(-c4c(Cl)c(C)cc5[nH]ncc45)c3C)C2)C1] | Using Method-1 from Intermediate C24a and D4 (Step 1) and C-SFC-2 (mobile phase: CO₂/[IPA + 0.025% NH₃] 65/35) to separate the 2 isomers at Step 2 to give the first eluting isomer: Intermediate 3-Int-1: C- SFC-3 (mobile phase: CO₂/[IPA + 0.025% NH₃] 65/35): Rt = 1.40 min and the second eluting isomer Intermediate 3-Int-2 = 2^{nd} eluting: C-SFC-3 (mobile phase: CO₂/[IPA + 0.025% NH₃] 65/35): Rt = 2.48 min. | Example 3a: prepared from Intermediate 3-Int-2: ¹H NMR (600 MHz, DMSO- d₆) δ 13.09 (s, 1H), 8.45- 8.30 (m, 2H), 7.63 (s, 1H), 7.58-7.51 (m, 1H), 7.46 (s, 1H), 7.29-7.21 (m, 1H), 6.37-6.22 (m, 1H), 6.15- 6.02 (m, 1H), 5.73-5.61 (m, 1H), 4.77-4.62 (m, 1H), 4.34 (s, 1H), 4.28 (s, 1H), 4.05 (s, 1H), 3.99 (s, 1H), 3.44-3.37 (m, 1H), 3.30- 3.23 (m, 1H), 3.13-3.04 (m, 1H), 2.95-2.72 (m, 4H), 2.71-2.63 (m, 2H), 2.48 (s, 3H), 2.14-2.03 (m, 1H), 1.92 (s, 3H), 1.84-1.73 (m, 1H); UPLC-MS-1a: Rt = 0.88 min; MS m/z [M + H]⁺ 542.3/544.3; Example 3b: |
| | | | prepared from |
| | 1-(6-(4-(5-chloro-6- | | Intermediate 3-Int-1: |
| | methyl-1H-indazol-4- | | UPLC-MS-1a: Rt = 0.88 |
| | yl)-5-methyl-3-(3- | | min; MS m/z [M + H]⁺ |
| | (pyridin-3-yl)pyrrolidin- | | 542.3/544.3. |
| | 1-yl)-1H-pyrazol-1-yl)-2- | | |
| | azaspiro[3.3]heptan-2- | | |
| | yl)prop-2-en-1-one | | |
| 4a/4b | 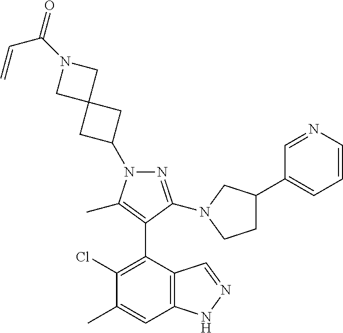 [SMILES: C=CC(=O)N1CC2(CC(n3nc(N4CCC(c5cccnc5)C4)c(-c4c(Cl)c(C)cc5[nH]ncc45)c3C)C2)C1] | Using Method-1 from Intermediate C24b and D4 (Step 1) and C-HPLC-10 (mobile phase: [n- heptane/IPA 80/20] + 0.05%DEA) to separate the 2 isomers at Step 2 to give the first eluting isomer: Intermediate 4-Int-1: C-HPLC-9 (mobile phase: [n- heptane + 0.1%DEA]/[IPA + 0.1%DEA] 80/20) Rt = 4.27 min and the second eluting isomer: Intermediate 4-Int- 2: C-HPLC-9 (mobile phase: [n- heptane + 0.1%DEA]/[IPA + 0.1%DEA] 80/20) Rt = 6.27 min. | Example 4a: prepared from Intermediate 4-Int-2: ¹H NMR (600 MHz, DMSO- d₆). δ 13.11 (s, 1H), 8.47- 8.32 (m, 2H), 7.64 (s, 1H), 7.63-7.59 (m, 1H), 7.46 (s, 1H), 7.31-7.25 (m, 1H), 6.35-6.24 (m, 1H), 6.14- 6.05 (m, 1H), 5.71-5.60 (m, 1H), 4.76-4.62 (m, 1H), 4.34 (s, 1H), 4.28 (s, 1H), 4.05 (s, 1H), 3.99 (s, 1H), 3.30-3.21 (m, 1H), 3.08-2.97 (m, 2H), 2.96- 2.87 (m, 1H), 2.83-2.72 (m, 2H), 2.71-2.62 (m, 3H), 2.46 (s, 3H), 2.16- 2.04 (m, 1H), 1.91 (s, 3H), 1.81-1.68 (m, 1H); UPLC- MS-1a: Rt = 0.88 min; MS m/z [M + H]⁺ 542.3/544.3, |
| | | | Example 4b: prepared |
| | 1-(6-(4-(5-chloro-6- | | from Intermediate 4-Int-1: |
| | methyl-1H-indazol-4- | | UPLC-MS-1a: Rt = 0.88 |
| | yl)-5-methyl-3-(3- | | min; MS m/z [M + H]⁺ |
| | (pyridin-3-yl)pyrrolidin- | | 542.3/544.2. |
| | 1-yl)-1H-pyrazol-1-yl)-2- | | |
| | azaspiro[3.3]heptan-2- | | |
| | yl)prop-2-en-1-one | | |
| 5 | 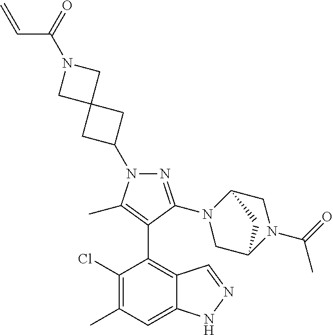 [SMILES: C=CC(=O)N1CC2(CC(n3nc(N4C[C@H]5C[C@@H]4CN5C(C)=O)c(-c4c(Cl)c(C)cc5[nH]ncc45)c3C)C2)C1] | Using Method-1 from Intermediate C20 and D4 without separation of the isomers (Step 1). | Example 5: ¹H NMR (600 MHz, DMSO-d₆) δ Mixture of atropisomers and rotamers; 13.1 (m, 1H), 7.61 (s, 1H), 7.50 (s, 1H), 6.31 (m, 1H), 6.10 (m, 1H), 5.68 (m, 1H), 4.70 (m, 1H), 4.44 (m, 0.5H), 4.34 (s, 1H), 4.33 (m, 0.5H), 4.29 (m, 1H), 4.04 (s, 1H), 3.99 (s, 1H), 3.86 (m, 0.5H), 3.68 (m, 0.5H), 3.37 (m, 0.5H), 3.27 (m, 0.5H), 3.16 (m, 0.5H), 3.03-2.80 (m, 1.5H), 2.79-2.65 (m, 4H), 2.50 (s, 3H), 1.93-1.83 (m, 4.5H), 1.73 (m, 1.5H), 1.62 (m, 2H); UPLC-MS-1g: Rt = 0.78 min; MS m/z [M + H]⁺ 534.2/536.2. |
| | 1-(6-(3-((1R,4R)-5- | | |
| | acetyl-2,5- | | |
| | diazabicyclo[2.2.1] | | |
| | heptan-2-yl)-4-(5-chloro- | | |
| | 6-methyl-1H-indazol-4- | | |
| | yl)-5-methyl-1H- | | |
| | pyrazol-1-yl)-2- | | |
| | azaspiro[3.3]heptan-2- | | |
| | yl)prop-2-en-1-one | | |
| 6 | 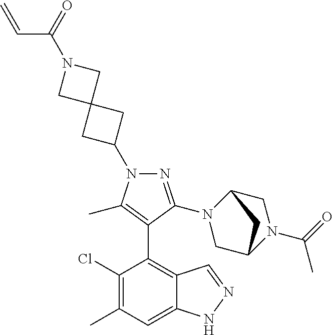 [SMILES: C=CC(=O)N1CC2(CC(n3nc(N4C[C@@H]5C[C@H]4CN5C(C)=O)c(-c4c(Cl)c(C)cc5[nH]ncc45)c3C)C2)C1] | Using Method-1 from Intermediate C21 and D4 without separation of the isomers (Step 1). | Example 6: ¹H NMR (600 MHz, DMSO-d₆) δ Mixture of atropisomers and rotamers; 13.12 (m, 1H), 7.61 (s, 1H), 7.50 (s, 1H), 6.31 (m, 1H), 6.10 (m, 1H), 5.68 (m, 1H), 4.68 (m, 1H), 4.44 (m, 0.5H), 4.34 (s, 1H), 4.33 (m, 0.5H), 4.28 (m, 1H), 4.04 (s, 1H), 3.99 (m, 1H), 3.86 (m, 0.5H), 3.67 (m, 0.5H), 3.27 (m, 1H), 3.16 (m, 1H), 3.04- 2.80 (m, 1.5H), 2.77-2.65 (m, 4.5H), 2.50 (s, 3H), 1.91-1.85 (m, 4.5H), 1.73 (m, 1.5H), 1.69-1.52 (m, 2H); UPLC-MS-1g: Rt = 0.77 min; MS m/z [M + H]⁺ 534.2/536.2. |
| | 1-(6-(3-((1S,4S)-5- | | |
| | acetyl-2,5- | | |
| | diazabicyclo[2.2.1] | | |
| | heptan-2-yl)-4-(5-chloro- | | |
| | 6-methyl-1H-indazol-4- | | |
| | yl)-5-methyl-1H- | | |
| | pyrazol-1-yl)-2- | | |
| | azaspiro[3.3]heptan-2- | | |
| | yl)prop-2-en-1-one | | |
| 7a/7b | 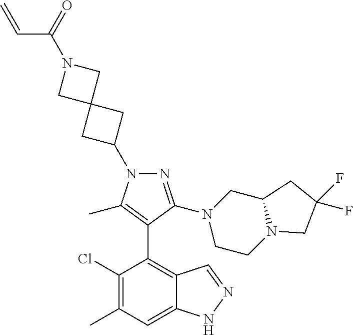 [SMILES: C=CC(=O)N1CC2(CC(n3nc(N4CCN5CC(F)(F)C[C@H]5C4)c(-c4c(Cl)c(C)cc5[nH]ncc45)c3C)C2)C1] | Using Method-1 from Intermediate C22 and D4 (Step 1) and C-SFC-2: (mobile phase: CO₂/[IPA + 0.1%Et₃N] 78/22) to separate the 2 isomers at Step 2 to give the first eluting isomer: Intermediate 7-Int-1 and the seond eluting isomer: Intermediate 7-Int-2. | Example 7a: prepared from Intermediate 7-Int-2: ¹H NMR (600 MHz, DMSO- d₆) δ 13.06 (s, 1H), 7.63 (s, 1H), 7.48 (s, 1H), 6.31 (m, 1H), 6.10 (m, 1H), 5.68 (m, 1H), 4.70 (m, 1H), 4.35 (s, 1H), 4.28 (s, 1H), 4.05 (s, 1H), 3.99 (s, 1H), 3.54 (m, 1H), 3.25 (m, 1H), 2.82- 2.71 (m, 3H), 2.69-2.63 (m, 2H), 2.57-2.46 (m, 3H), 2.49 (s, 3H), 2.44-2.35 (m, 2H), 2.23 (m, 1H), 1.94 (s, 3H), 1.92-1.82 (m, 1H), 1.72 (m, 1H). UPLC-MS-1f: Rt = 4.46 min; MS m/z [M + H]⁺ 556.2/ 558.2. Example 7b: prepared from Intermediate 7-Int-1: UPLC-MS-1f: Rt = 4.52 min; MS m/z [M + H]⁺ 556.2/558.2. |
| | (S)-1-(6-(4-(5-chloro-6- | | |
| | methyl-1H-indazol-4- | | |
| | yl)-3-(7,7- | | |
| | difluorohexahydropyrrolo | | |
| | [1,2-a]pyrazin-2(1H)- | | |
| | yl)-5-methyl-1H- | | |
| | pyrazol-1-yl)-2- | | |
| | azaspiro[3.3]heptan-2- | | |
| | yl)prop-2-en-1-one | | |
| 8a/8b | 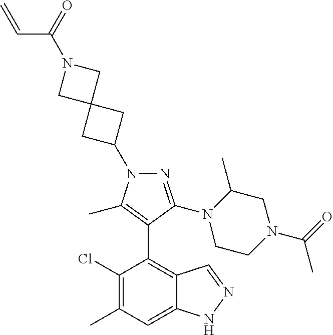 [SMILES: C=CC(=O)N1CC2(CC(n3nc(N4CCN(C(C)=O)CC4C)c(-c4c(Cl)c(C)cc5[nH]ncc45)c3C)C2)C1] | Using Method-1 from Intermediate C23a and D4 (Step 1) and C-SFC-4: (mobile phase: CO₂/[IPA + 0.1%Et₃N] 75/25) to separate the 2 isomers at Step 2 to give the first eluting isomer: Intermediate 8-Int-1 and Intermediate the second eluting isomer 8-Int- 2. | Example 8a: prepared from Intermediate8-Int-2: ¹H NMR (600 MHz, DMSO- d₆) δ Mixture of rotamers; 13.07 (m, 1H), 7.64 (s, 1H), 7.48 (s, 1H), 6.31 (dd, 1H), 6.11 (d, 1H), 5.68 (d, 1H), 4.71 (m, 1H), 4.34 (s, 1H), 4.28 (s, 1H), 4.05 (s, 1H), 3.99 (s, 1H), 3.86 (m, 0.5H), 3.49-3.41 (m, 1H), 3.16 (m, 1H), 3.13-3.06 (m, 1H), 3.02 (m, 1H), 2.91 (m, 1H), 2.83 (m, 1H), 2.79- 2.64 (m, 4.5H), 2.50 (s, 3H), 1.95 (s, 3H), 1.95 (s, 1.5H), 1.85 (s, 1.5H), 0.89- 0.73 (m, 3H); UPLC-MS- 1h: Rt = 4.27 min; MS m/z [M + H]⁺ 536.2/538.2; C- SFC-3 (mobile phase: |
| | | | CO₂/[IPA + 0.025%NH₃] 5 to |
| | 1-(6-(3-(4-Acetyl-2- | | 55%): Rt = 3.97 min. |
| | methylpiperazin-1-yl)-4- | | Example 8b: prepared |
| | (5-chloro-6-methyl-1H- | | from Intermediate 8-Int-1: |
| | indazol-4-yl)-5-methyl- | | C-SFC-3 (mobile phase: |
| | 1H-pyrazol-1-yl)-2- | | CO₂/[IPA + 0.025%NH₃] 5 to |
| | azaspiro[3.3]heptan-2- | | 55%): Rt = 3.57 min. |
| | yl)prop-2-en-1-one | | |
| 9a/9b | 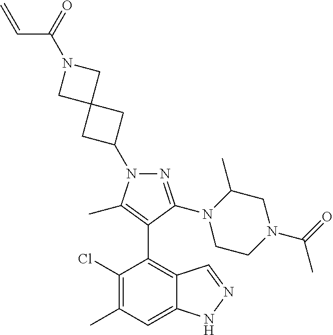 [SMILES: C=CC(=O)N1CC2(CC(n3nc(N4CCN(C(C)=O)CC4C)c(-c4c(Cl)c(C)cc5[nH]ncc45)c3C)C2)C1] | Using Method-1 from Intermediate C23b and D4 (Step 1) and C-SFC-2: (mobile phase: CO₂/IPA 70/30) to separate the 2 isomers at Step 2 to give the first eluting isomer: Intermediate 9-Int-1 and Intermediate the second eluting isomer 9-Int-2. | Example 9a: prepared from Intermediate 9_Int-2: ¹H NMR (600 MHz, DMSO- d₆) δ Mixture of rotamers; 13.1 (s, 1H), 7.63 (s, 1H), 7.49 (s, 1H), 6.31 (dd, 1H), 6.10 (d, 1H), 5.68 (d, 1H), 4.71 (m, 1H), 4.34 (s, 1H), 4.28 (s, 1H), 4.05 (s, 1H), 3.99 (s, 1H), 3.81 (m, 0.5H), 3.56 (m, 0.5H), 3.40 (m, 0.5H), 3.30-3.17 (m, 2H), 3.08-2.88 (m, 2.5H), 2.86-2.73 (m, 2H), 2.72- 2.65 (m, 3H), 2.50 (s, 3H), 1.94 (s, 1.5H), 1.91 (s, 1.5H), 1.90 (s, 1.5H), 1.87 (s, 1.5H), 0.76 (m, 3H); UPLC-MS-1h: Rt = 4.28 min; MS m/z [M + H]⁺ 536.2/ 538.2; C-SFC-3 (mobile |
| | | | phase: |
| | 1-(6-(3-(4-Acetyl-2- | | CO₂/[IPA + 0.025%NH₃] 5 to |
| | methylpiperazin-1-yl)-4- | | 55%): Rt = 4.01 min. |
| | (5-chloro-6-methyl-1H- | | Example 9b: prepared |
| | indazol-4-yl)-5-methyl- | | from Intermediate 9-Int-1: |
| | 1H-pyrazol-1-yl)-2- | | C-SFC-3 (mobile phase: |
| | azaspiro[3.3]heptan-2- | | CO₂/[IPA + 0.025%NH₃] 5 |
| | yl)prop-2-en-1-one | | to 55%): Rt = 3.58 min. |

### Method-2 for the Preparation of Example 10a and 10b: 1-(6-(4-(5-Chloro-6-methyl-1H-indazol-4-yl)-3-(6,7-dihydropyrazolo[1,5-a]pyrazin-5(4H)-yl)-5-methyl-1H-pyrazol-1-yl)-2-azaspiro[3.3]heptan-2-yl)prop-2-en-1-one

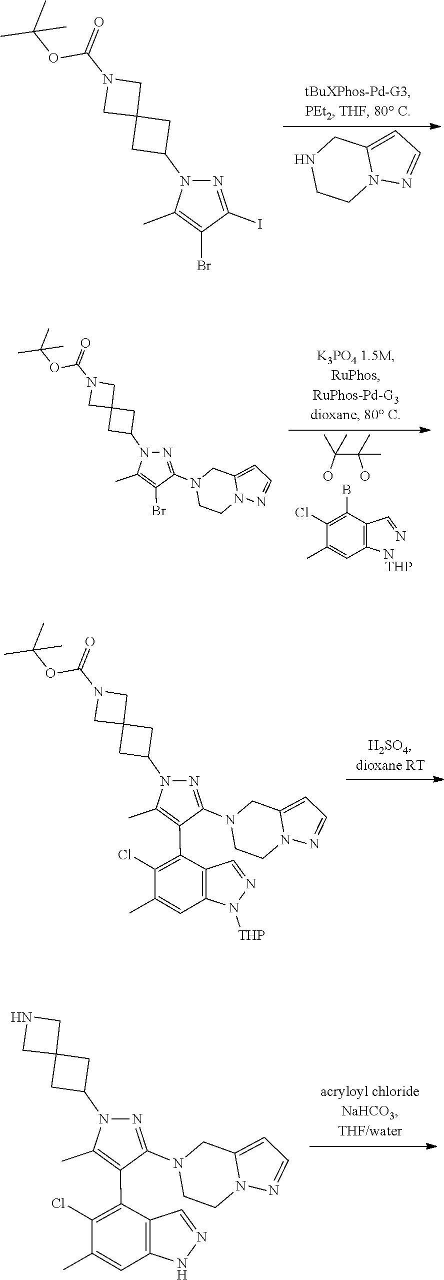

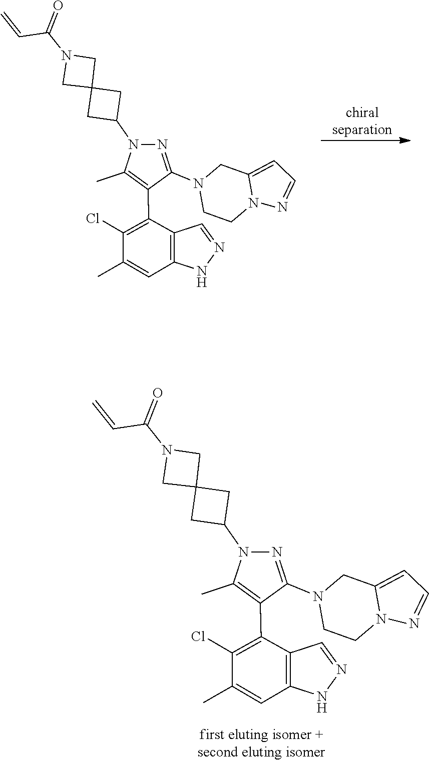 [SMILES: C=CC(=O)N1CC2(CC(n3nc(N4CCn5nccc5C4)c(-c4c(Cl)c(C)cc5[nH]ncc45)c3C)C2)C1 ; C=CC(=O)N1CC2(CC(n3nc(N4CCn5nccc5C4)c(-c4c(Cl)c(C)cc5[nH]ncc45)c3C)C2)C1]

### Step 1: Tert-butyl 6-(4-bromo-3-(6,7-dihydropyrazolo[1,5-a]pyrazin-5(4H)-yl)-5-methyl-1H-pyrazol-1-yl)-2-azaspiro[3.3]heptane-2-carboxylate

In a vial, to a solution of tert-butyl 6-(4-bromo-3-iodo-5-methyl-1H-pyrazol-1-yl)-2-azaspiro[3.3]heptane-2-carboxylate (Intermediate C5, 300 mg, 0.62 mmol), 4,5,6,7-tetrahydropyrazolo[1,5-a]pyrazine dihydrochloride (207 mg, 1.06 mmol), tBuXPhos-Pd-G3 (49.4 mg, 0.062 mmol) in THF (4.5 mL) was added Phosphazene P₂-Et (CAS: 165535-45-5, 0.93 mL, 2.80 mmol). The vial was flushed with argon and stirred at 80° C. for 15 h. The reaction mixture was poured into water and extracted with CH₂Cl₂ (×3). The combined organic extracts were dried (phase separator) and evaporated. The crude residue was purified by normal flash chromatography (eluent: EtOAc in c-hexane 0 to 60%) to give the title compound as a yellow foam. UPLC-MS-1a: Rt=1.22 min; MS m/z [M+H]⁺ 477.3/479.3.

### Step 2: Tert-butyl 6-(4-(5-chloro-6-methyl-1-(tetrahydro-2H-pyran-2-yl)-1H-indazol-4-yl)-3-(6,7-dihydropyrazolo[1,5-a]pyrazin-5(4H)-yl)-5-methyl-1H-pyrazol-1-yl)-2-azaspiro[3.3]heptane-2-carboxylate

In a vial were added tert-butyl 6-(4-bromo-3-(6,7-dihydropyrazolo[1,5-a]pyrazin-5(4H)-yl)-5-methyl-1H-pyrazol-1-yl)-2-azaspiro[3.3]heptane-2-carboxylate (Step 1, 235 mg, 0.49 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 185 mg, 0.49 mmol), RuPhos (11.5 mg, 0.025 mmol) and RuPhos-Pd-G3 (20.6 mg, 0.025 mmol). The vial was flushed with argon, dioxane (3.70 mL) and K₃PO₄ (1.5 M in water, 0.98 mL, 1.48 mmol) were added and the reaction mixture was stirred at 80° C. for 4 h. The reaction mixture was poured into a sat. aq. NaHCO₃ solution and extracted with EtOAc (×3). The combined organic extracts were dried (phase separator) and evaporated. The crude residue was diluted in THF (4 mL), SiliaMetS®Thiol (0.10 mmol) was added and the mixture swirled for 1 h at 40° C. The mixture was filtered, the filtrate was concentrated and the crude residue was purified by normal phase chromatography (eluent: MeOH in CH₂Cl₂ from 0 to 4%) to give the title compound as white foam. UPLC-MS-1a: Rt=1.41 and 1.43 min; MS m/z [M+H]⁺ 647.4/649.4.

### Step 3: 5-(4-(5-Chloro-6-methyl-1H-indazol-4-yl)-5-methyl-1-(2-azaspiro[3.3]heptan-6-yl)-1H-pyrazol-3-yl)-4,5,6,7-tetrahydropyrazolo[1,5-a]pyrazine

To a solution of tert-butyl 6-(4-(5-chloro-6-methyl-1-(tetrahydro-2H-pyran-2-yl)-1H-indazol-4-yl)-3-(6,7-dihydropyrazolo[1,5-a]pyrazin-5(4H)-yl)-5-methyl-1H-pyrazol-1-yl)-2-azaspiro[3.3]heptane-2-carboxylate (Step 2, 143 mg, 0.22 mmol) in dioxane (2 mL) was added H₂SO₄ (0.21 mL, 3.98 mmol) and the reaction mixture was stirred at RT for 16 h. The reaction mixture was diluted with CH₂Cl₂ and poured into water. The layers were separated and the aqueous one was back extracted with CH₂Cl₂. NaOH (1N aq., 13.3 mL) and CH₂Cl₂ were added to the aqueous layer, the layers were separated and the aqueous layer was back extracted with CH₂Cl₂ (×2). The combined organic extracts were dried (phase separator) and concentrated in vacuo to give the title compound as a beige foam. UPLC-MS-1a: Rt=0.68 min; MS m/z [M+H]⁺ 463.3/465.2.

### Step 4: 1-(6-(4-(5-Chloro-6-methyl-1H-indazol-4-yl)-3-(6,7-dihydropyrazolo[1,5-a]pyrazin-5(4H)-yl)-5-methyl-1H-pyrazol-1-yl)-2-azaspiro[3.3]heptan-2-yl)prop-2-en-1-one Example 10a and Example 10b

To an ice-cooled solution of 5-(4-(5-chloro-6-methyl-1H-indazol-4-yl)-5-methyl-1-(2-azaspiro[3.3]heptan-6-yl)-1H-pyrazol-3-yl)-4,5,6,7-tetrahydropyrazolo[1,5-a]pyrazine (Step 3, 65.0 mg, 0.14 mmol) and NaHCO₃ (54.3 mg, 0.65 mmol) in THF (5.20 mL) and water (1.16 mL) was added acryloyl chloride (0.02 mL, 0.26 mmol). The reaction mixture was stirred at 0-5° C. under nitrogen atmosphere for 1.15 h. Acryloyl chloride (5 μL, 0.06 mmol) was added again to complete the reaction. MeOH (0.5 mL) was added at 0-5° C. and the mixture was stirred for 1.5 h until disappearance (UPLC) of the side product resulting from reaction of the acryloyl chloride with the indazole NH. The reaction mixture was poured into a sat. aq. NaHCO₃ solution and extracted with CH₂Cl₂ (×3). The combined organic extracts were dried (phase separator) and evaporated. The crude residue was purified by normal phase chromatography (eluent: MeOH in CH₂Cl₂ 0 to 6%) to give the title compound. The isomers were separated by chiral SFC (C-SFC-5; mobile phase: CO₂/MeOH 63/37) to give after lyophilization (CH₃CN/water) the title compound Example 10b as the second eluting peak (white solid): ¹H NMR (600 MHz, DMSO-d₆) δ 13.1 (s, 1H), 7.63 (s, 1H), 7.53 (s, 1H), 7.32 (s, 1H), 6.31 (m, 1H), 6.10 (m, 1H), 5.98 (s, 1H), 5.72 (m, 1H), 4.73 (m, 1H), 4.34 (s, 1H), 4.27-4.25 (m, 2H), 4.19 (dd, 1H), 4.05 (s, 1H), 4.00 (s, 1H), 3.85 (m, 1H), 3.62 (m, 1H), 3.21-3.12 (m, 2H), 2.78-2.66 (m, 4H), 2.50 (s, 3H), 1.96 (s, 3H). UPLC-MS-1 b: Rt=4.45 min; MS m/z [M+H]⁺ 517.3/519.3. C-SFC-6 (mobile phase: CO₂/MeOH 60/40): Rt=2.40 min. The other isomer Example 10a was obtained as the first eluting peak: C-SFC-6 (mobile phase: CO₂/MeOH 60/40): Rt=1.45 min.

Method-2a: similar to Method-2 except that Step 3 was performed using TFA in CH₂Cl₂ as described in Method-3 Step 4.

The following examples 11a to 12 were prepared using analogous methods to method-2 from intermediates (in Step 1) described in the intermediates synthesis section or commercially available.

| | | Method, | |
|---|---|---|---|
| | | intermediates and | |
| | | chiral separation | |
| | | conditions used | |
| Example | Structure | and order of elution | Characterizing data |
| 11a/11b | 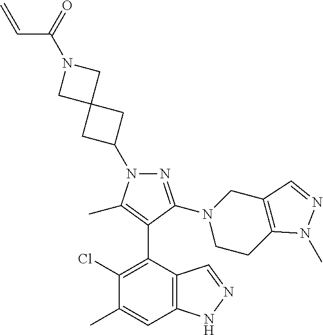 [SMILES: C=CC(=O)N1CC2(CC(n3nc(N4CCc5c(cnn5C)C4)c(-c4c(Cl)c(C)cc5[nH]ncc45)c3C)C2)C1] | Using Method-2 from 1-methyl-4,5,6,7- tetrahydro-1H- pyrazolo[4,3- c]pyridine hydrochloride and C- SFC-7 (mobile phase: CO₂/MeOH 60/40); Example 11a = 1^{st} eluting isomer, Example 11b = 2^{nd} eluting isomer; | Example 11b: ¹H NMR (600 MHz, DMSO-d₆) δ 13.1 (s, 1H), 7.59 (s, 1H), 7.50 (s, 1H), 7.11 (s, 1H), 6.32 (m, 1H), 6.10 (m, 1H), 5.68 (m, 1H), 4.70 (m, 1H), 4.34 (s, 1H), 4.28 (s, 1H), 4.02- 3.98 (m, 2H), 3.91 (m, 1H), 3.55 (s, 3H), 3.01-2.95 (m, 2H), 2.78- 2.73 (m, 2H), 2.69-2.64 (m, 2H), 2.50 (s, 3H), 2.37 (m, 1H), 2.15 (m, 1H), 1.93 (s, 3H). UPLC- MS-1b: Rt = 4.41 min; MS m/z [M + H]⁺ 531.3/533.3; C-SFC-8 (mobile phase: CO₂/MeOH 60/40): Rt = 3.00 min, Example 11a: C-SFC-8 (mobile phase: CO₂/MeOH 60/40): Rt = 2.29 min. |
| | 1-(6-(4-(5-chloro-6- | | |
| | methyl-1H-indazol-4-yl)- | | |
| | 5-methyl-3-(1-methyl- | | |
| | 1,4,6,7-tetrahydro-5H- | | |
| | pyrazolo[4,3-c]pyridin-5- | | |
| | yl)-1H-pyrazol-1-yl)-2- | | |
| | azaspiro[3.3]heptan-2- | | |
| | yl)prop-2-en-1-one | | |
| 12 | 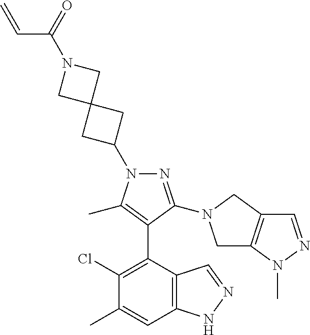 [SMILES: C=CC(=O)N1CC2(CC(n3nc(N4Cc5cnn(C)c5C4)c(-c4c(Cl)c(C)cc5[nH]ncc45)c3C)C2)C1] | Using Method-2a from 1-methyl- 1,4,5,6- tetrahydropyrrolo[3,4- c]pyrazole Intermediate A57 | Example 12: ¹H NMR (400 MHz, DMSO-d₆) δ 13.16 (s, 1H), 7.65 (s, 1H), 7.54 (s, 1H), 7.01 (s, 1H), 6.37 (m, 1H), 6.14 (d, 1H), 5.70 (d, 1H), 4.75 (m, 1H), 4.34 (s, 1H), 4.33 (m, 2H), 4.31 (s, 1H), 4.08 (s, 1H), 4.02 (s, 1H), 3.83 (d, 1H), 3.70 (s, 3H), 3.59 (d, 1H), 2.85-2.75 (m, 2H), 2.69 (m, 2H), 2.50 (s, 3H), 1.93 (s, 3H). UPLC-MS-7: Rt = 4.16 min, MS m/z [M + H]⁺ 517.2/519.2; RP-HPLC-10: Rt = 5.88 min. |
| | 1-(6-(4-(5-chloro-6- | | |
| | methyl-1H-indazol-4-yl)- | | |
| | 5-methyl-3-(1-methyl- | | |
| | 4,6-dihydropyrrolo[3,4- | | |
| | c]pyrazol-5(1H)-yl)-1H- | | |
| | pyrazol-1-yl)-2- | | |
| | azaspiro[3.3]heptan-2- | | |
| | yl)prop-2-en-1-one | | |

### Method-3 for the Preparation of Example 13a and 13b: 1-(6-(3-((2S,6R)-4-Acetyl-2,6-dimethylpiperazin-1-yl)-4-(5-chloro-6-methyl-1H-indazol-4-yl)-5-methyl-1H-pyrazol-1-yl)-2-azaspiro[3.3]heptan-2-yl)prop-2-en-1-one

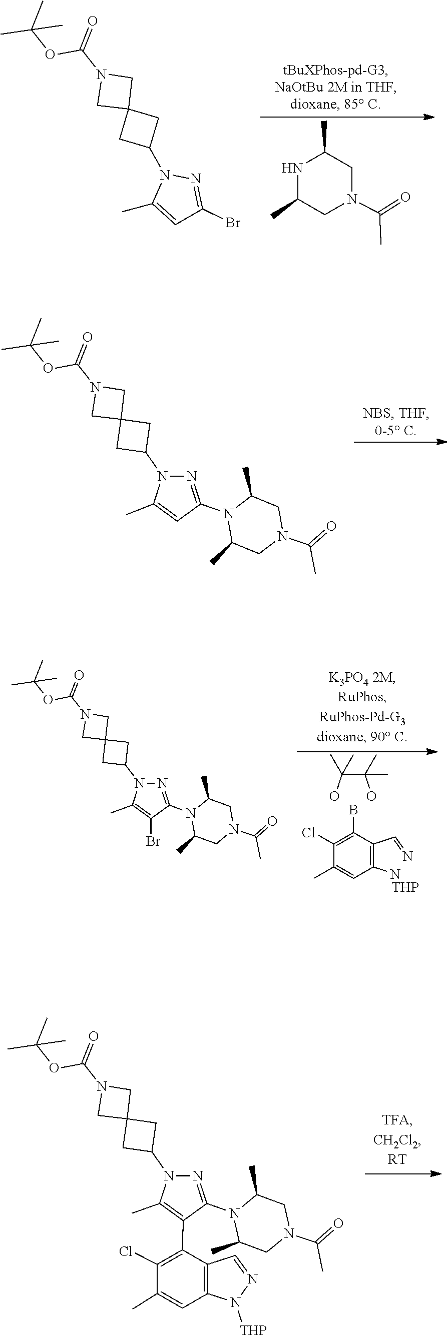

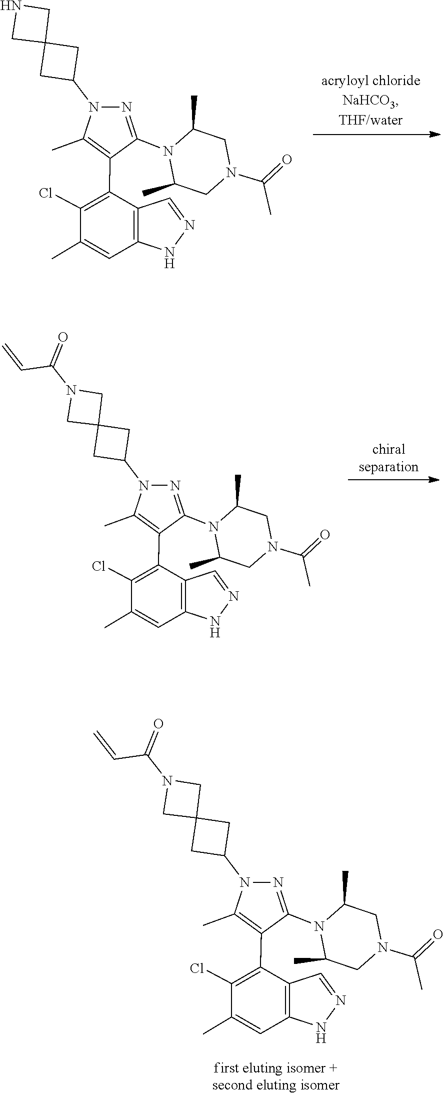 [SMILES: CC(=O)N1C[C@@H](C)N(c2nn(C3CC4(CNC4)C3)c(C)c2-c2c(Cl)c(C)cc3[nH]ncc23)[C@@H](C)C1 ; C=CC(=O)N1CC2(CC(n3nc(N4[C@H](C)CN(C(C)=O)C[C@@H]4C)c(-c4c(Cl)c(C)cc5[nH]ncc45)c3C)C2)C1 ; C=CC(=O)N1CC2(CC(n3nc(N4[C@H](C)CN(C(C)=O)C[C@@H]4C)c(-c4c(Cl)c(C)cc5[nH]ncc45)c3C)C2)C1]

### Step 1: Tert-butyl 6-(3-((2R,6S)-4-acetyl-2,6-dimethylpiperazin-1-yl)-5-methyl-1H-pyrazol-1-yl)-2-azaspiro[3.3]heptane-2-carboxylate

To a solution of tert-butyl 6-(3-bromo-5-methyl-1H-pyrazol-1-yl)-2-azaspiro[3.3]heptane-2-carboxylate (Intermediate C1, 1.40 g, 3.93 mmol) and 1-((3R,5S)-3,5-dimethylpiperazin-1-yl)ethan-1-one (Intermediate A10, 0.74 g, 4.72 mmol) in dioxane (18 mL) under nitrogen atmosphere were added tBuXPhos-Pd-G3 (0.25 g, 0.31 mmol) and NaOtBu (2M in THF, 5.89 mL, 11.8 mmol). The reaction mixture was stirred at 85° C. for 16 h. The RM was quenched by addition a sat. aq. NaHCO₃ solution and extracted with AcOEt (×2). The combined organic extracts were dried (Na₂SO₄), filtered and evaporated. The crude residue was purified by normal phase chromatography (eluent: MeOH in CH₂Cl₂ from 0 to 4%) to give the title compound as a beige foam. UPLC-MS-2b: Rt=1.01 min; MS m/z [M+H]⁺ 432.6.

### Step 2: Tert-butyl 6-(3-((2R,6S)-4-acetyl-2,6-dimethylpiperazin-1-yl)-4-bromo-5-methyl-1H-pyrazol-1-yl)-2-azaspiro[3.3]heptane-2-carboxylate

To a solution of tert-butyl 6-(3-((2R,6S)-4-acetyl-2,6-dimethylpiperazin-1-yl)-5-methyl-1H-pyrazol-1-yl)-2-azaspiro[3.3]heptane-2-carboxylate (Step 1, 234 mg, 0.54 mmol) in THF (5.42 mL) at 0° C. under nitrogen atmosphere was added NBS (101 mg, 0.57 mmol) and the reaction mixture was stirred at 0° C. for 1 h. The RM was poured into water, extracted with CH₂Cl₂ (2×) and the combined organic extracts were washed with a sat. aq. NaHCO₃ solution, dried (Na₂SO₄), filtered and evaporated to give the title compound as a beige foam. UPLC-MS-2b: Rt=1.12 min; MS m/z [M+H]⁺ 510.2/512.2.

### Step 3: Tert-butyl 6-(3-((2R,6S)-4-acetyl-2,6-dimethylpiperazin-1-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate

To tert-butyl 6-(3-((2R,6S)-4-acetyl-2,6-dimethylpiperazin-1-yl)-4-bromo-5-methyl-1H-pyrazol-1-yl)-2-azaspiro[3.3]heptane-2-carboxylate (Step 2, 180 mg, 0.35 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 159 mg, 0.42 mmol), RuPhos (16.5 mg, 0.035 mmol) and RuPhos-Pd-G3 (29.5 mg, 0.035 mmol) in 1,4-dioxane (1.41 mL) were added K₃PO₄ (225 mg, 1.06 mmol) and water (353 μL). The reaction mixture was degassed and stirred at 90° C. for 1 h. The RM was quenched by addition of a sat. aq. NaHCO₃ solution and extracted AcOEt (2×). The combined organic extracts were dried (Na₂SO₄), filtered and evaporated. The crude residue was diluted in THF (5 mL), SiliaMetS®Thiol (0.14 mmol) was added and the mixture was swirled at 40° C. for 1 h. The mixture was filtered, washed with THF and the filtrate was concentrated to dryness under reduced pressure. The residue was purified by normal phase chromatography (eluent: MeOH in CH₂Cl₂ from 0 to 5%) to give the title compound as a light yellow foam. UPLC-MS-2b: Rt=1.21 min; MS m/z [M+H]⁺ 680.3/682.3.

### Step 4: 1-(6-(3-((2R,6S)-4-Acetyl-2,6-dimethylpiperazin-1-yl)-4-(5-chloro-6-methyl-1H-indazol-4-yl)-5-methyl-1H-pyrazol-1-yl)-2-azaspiro[3.3]heptan-2-yl)prop-2-en-1-one

To a solution of tert-butyl 6-(3-((2R,6S)-4-acetyl-2,6-dimethylpiperazin-1-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Step 3, 83 mg, 0.12 mmol) in CH₂Cl₂ (610 μL) was added TFA (282 μL, 3.66 mmol) and the reaction mixture was stirred at RT for 1 h. The reaction mixture was evaporated to dryness, then diluted with dioxane, frozen and lyophilized to give the title compound as a trifluoroacetate salt. UPLC-MS-2b: Rt=0.71 min; MS m/z [M+H]⁺ 496.2/498.2.

### Step 5: 1-(6-(3-((2S,6R)-4-Acetyl-2,6-dimethylpiperazin-1-yl)-4-(5-chloro-6-methyl-1H-indazol-4-yl)-5-methyl-1H-pyrazol-1-yl)-2-azaspiro[3.3]heptan-2-yl)prop-2-en-1-one

To a solution of 1-(6-(3-((2R,6S)-4-acetyl-2,6-dimethylpiperazin-1-yl)-4-(5-chloro-6-methyl-1H-indazol-4-yl)-5-methyl-1H-pyrazol-1-yl)-2-azaspiro[3.3]heptan-2-yl)prop-2-en-1-one trifluoroacetate (Step 4, 0.12 mmol) in THF (4.8 mL) at 0° C. were added NaHCO₃ (0.5M aq., 2.44 mL, 1.22 mmol) and a solution of acryloyl chloride (10.4 μL, 0.13 mmol) diluted in THF (100 L). The reaction mixture was stirred at 0° C. for 1 h, then was diluted with CH₂Cl₂ and quenched with a sat. aq. NaHCO₃ solution. The layers were separated and the organic layer was dried (phase separator) and evaporated to dryness. The crude residue was purified by normal phase chromatography (eluent: MeOH in CH₂Cl₂ from 0 to 10%) to give the title compound. The isomers were separated by chiral SFC (C-SFC-2; mobile phase: CO₂/IPA 68/32) to give after lyophilization (CH₃CN/water) the title compound Example 13b as the second eluting peak (white solid): ¹H NMR (600 MHz, DMSO-d₆) δ 13.1 (s, 1H), 7.58 (s, 1H), 7.49 (s, 1H), 6.31 (m, 1H), 6.10 (m, 1H), 5.67 (m, 1H), 4.76 (m, 1H), 4.35 (s, 1H), 4.25 (s, 1H), 4.05 (s, 1H), 3.99 (m, 1H), 3.70 (m, 1H), 3.45 (m, 1H), 3.10-2.86 (m, 3H), 2.76-2.67 (m, 5H), 2.48 (s, 3H), 1.92-1.90 (m, 6H), 0.90-0.80 (m, 6H). UPLC-MS-2d: Rt=4.00 min; MS m/z [M+H]⁺ 550.2/552.3. C-SFC-3 (mobile phase: CO₂/IPA 68/32): Rt=2.05 min. The other isomer Example 13a was obtained as the first eluting peak: C-SFC-3 (mobile phase: CO₂/IPA 68/32): Rt=1.44 min.

Method-3a: similar to Method-3 except that Step 1 was performed using Phosphazene P₂-Et in THF instead of NaOtBu in dioxane as described in Method-2 Step 1.

Method-3b: similar to Method-3 except that in Step 2 NIS was used instead of NBS to prepare the corresponding 4-iodo-pyrazole.

Method-3c: similar to Method-3 except that in Step 2 NIS in acetonitrile was used instead of NBS in THF to prepare the corresponding 4-iodo-pyrazole.

Method-3d: similar to Method-3 except that Step 4 was performed using H₂SO₄ in dioxane as described in Method-2 Step 3.

Method-3e: similar to Method-3 except that in Step 2 NIS, AIBN (0.1 eq) in acetonitrile was used instead of NBS in THF to prepare the corresponding 4-iodo-pyrazole.

Method-3f: similar to Method-3 except that in Step 2 acetonitrile was used instead of THF.

Method-3g: similar to Method-3 except that in Step 1 THF was used instead of dioxane.

Method-3h: similar to Method-3 except Step 5 was performed using acrylic acid and T₃P in CH₂Cl₂ as described in Method-4 Step 5.

The following examples 14a to 53d were prepared using analogous methods to Method-3 from intermediates (in Step 1, 2, 3 or 4) described in the intermediates synthesis section or commercially available.

In Step 5, If observed the side product resulting from reaction of the acryloyl chloride with the indazole NH was hydrolyzed by addition of MeOH (0.5 mL) at 0-5° C. and stirring until disappearance (UPLC).

When the final separation of isomers is performed by preparative HPLC using acidic conditions the purified fractions were extracted with a sat. aq. NaHCO₃ solution before lyophilization from CH₃CN/H₂O to give the title compound as a free base.

| | | Method, | |
|---|---|---|---|
| | | intermediates (in | |
| | | Step 1, 2, 3 or 4) | |
| | | and chiral | |
| | | separation | |
| | | conditions used | |
| | | and order of | |
| Example | Structure | elution | Characterizing data |
| 14a/14b | 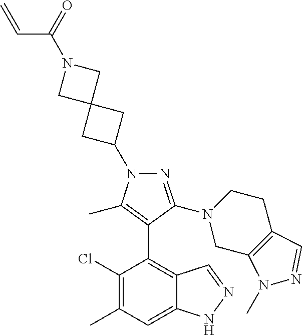 [SMILES: C=CC(=O)N1CC2(CC(n3nc(N4CCc5cnn(C)c5C4)c(-c4c(Cl)c(C)cc5[nH]ncc45)c3C)C2)C1] | Using Method- 3bd from CAS [1228878-69-0] (Step 1) and C- SFC-4 (mobile phase: CO₂/[IPA + 0.025% NH₃] 70/30); Example 14a = 1^{st} eluting isomer, Example 14b = 2^{nd} eluting isomer | Example 14b: ¹H NMR (600 MHz, DMSO-d₆) δ 13.1 (s, 1H), 7.59 (s, 1H), 7.51 (s, 1H), 7.04 (s, 1H), 6.31 (m, 1H), 6.10 (m, 1H), 5.67 (m, 1H), 4.70 (m, 1H), 4.34 (s, 1H), 4.30 (s, 1H), 4.23 (m, 1H), 4.13 (m, 1H), 4.04 (s, 1H), 4.01 (s, 1H), 3.59 (m, 3H), 3.02 (m, 1H), 2.83 (m, 1H), 2.79-2.66 (m, 4H), 2.50 (s, 3H), 2.13 (m, 1H), 1.95 (s, 3H), 1.89 (m, 1H). UPLC-MS-1b: Rt = 4.52 min; MS m/z [M + H]⁺ 531.3/533.3; C-SFC-11 (mobile phase: CO₂//[IPA + 0.1% Et₃N] 70/30): Rt = 2.34 min, Example 14a: C- SFC-11 (mobile phase: CO₂/[IPA + 0.1% Et₃N] 70/30): Rt = 1.72 min. |
| | 1-(6-(4-(5-chloro-6- | | |
| | methyl-1H-indazol-4-yl)- | | |
| | 5-methyl-3-(1-methyl- | | |
| | 1,4,5,7-tetrahydro-6H- | | |
| | pyrazolo[3,4-c]pyridin-6- | | |
| | yl)-1H-pyrazol-1-yl)-2- | | |
| | azaspiro[3.3]heptan-2- | | |
| | yl)prop-2-en-1-one | | |
| 15a/15b | 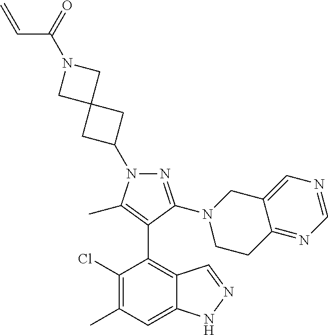 [SMILES: C=CC(=O)N1CC2(CC(n3nc(N4CCc5ncncc5C4)c(-c4c(Cl)c(C)cc5[nH]ncc45)c3C)C2)C1] | Using Method- 3acd from 5,6,7,8- tetrahydropyrido [4,3-d]pyrimidine dihydrochloride (Step 1) and C- SFC-9 (mobile phase: CO₂/MeOH 40/60); Example 15a = 1^{st} eluting isomer, Example 15b: = 2^{nd} eluting isomer | Example 15b: ¹H NMR (600 MHz, DMSO-d₆) δ 13.1 (s, 1H), 8.86 (m, 1H), 8.51 (m, 1H), 7.62 (s, 1H), 7.53 (s, 1H), 6.33 (m, 1H), 6.11 (m, 1H), 5.69 (m, 1H), 4.71 (m, 1H), 4.35-4.21 (m, 4H), 4.05 (s, 1H), 4.00 (s, 1H), 2.99 (m, 2H), 2.78-2.66 (m, 4H), 2.51 (m, 1H), 2.50 (s, 3H), 2.30 (m, 1H), 1.97 (s, 3H). UPLC-MS-2d: Rt = 3.87 min; MS m/z [M + H]⁺ 529.2/531.1; C-SFC-10 (mobile phase: CO₂/MeOH 50/50): Rt = 3.95 min, Example 15a: C-SFC- 10 (mobile phase: CO₂/MeOH 50/50): Rt = 3.26 min. |
| | 1-(6-(4-(5-chloro-6- | | |
| | methyl-1H-indazol-4-yl)- | | |
| | 3-(7,8-dihydropyrido[4,3- | | |
| | d]pyrimidin-6(5H)-yl)-5- | | |
| | methyl-1H-pyrazol-1-yl)- | | |
| | 2-azaspiro[3.3]heptan-2- | | |
| | yl)prop-2-en-1-one | | |
| 16 | 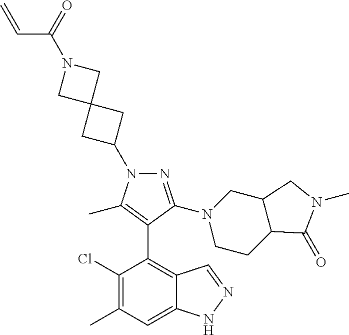 [SMILES: C=CC(=O)N1CC2(CC(n3nc(N4CCC5C(=O)N(C)CC5C4)c(-c4c(Cl)c(C)cc5[nH]ncc45)c3C)C2)C1] | Using Method-3cd from Intermediate C25a (Step 1) | Example 16: ¹H NMR (400 MHz, DMSO-d₆) δ 13.1 (s, 1H), 7.63 (s, 1H), 7.49 (s, 1H), 6.32 (m, 1H), 6.11 (m, 1H), 5.68 (m, 1H), 4.69 (m, 1H), 4.34 (s, 1H), 4.29 (s, 1H), 4.05 (s, 1H), 3.99 (s, 1H), 3.25 (m, 1H), 3.17 (dd, 1H), 3.08 (m, 1H), 2.83-2.62 (m, 8H), 2.50 (s, 3H), 2.45-2.39 (m, 2H), 2.31 (m, 1H), 2.22 (m, 1H), 1.94 (s, 3H), 1.56 (m, 1H), 1.43 (m, 1H). UPLC-MS-2d: Rt = 3.85 min; MS m/z [M − H]⁻ 548.2/550.2. |
| | (3aS*,7aS*)-5-(1-(2- | | |
| | acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 2-methyloctahydro-1H- | | |
| | pyrrolo[3,4-c]pyridin-1- | | |
| | one | | |
| 17 | 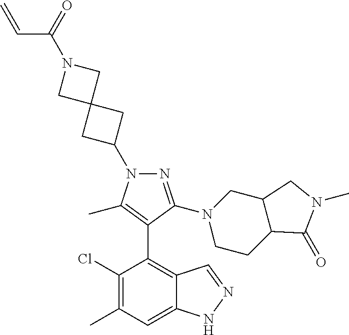 [SMILES: C=CC(=O)N1CC2(CC(n3nc(N4CCC5C(=O)N(C)CC5C4)c(-c4c(Cl)c(C)cc5[nH]ncc45)c3C)C2)C1] | Using Method-3c from Intermediate C25b (Step 1) | Example 17: ¹H NMR (600 MHz, DMSO-d₆) δ 13.1 (s, 1H), 7.63 (s, 1H), 7.49 (s, 1H), 6.31 (m, 1H), 6.11 (m, 1H), 5.68 (m, 1H), 4.69 (m, 1H), 4.34 (s, 1H), 4.29 (s, 1H), 4.06 (s, 1H), 3.99 (s, 1H), 3.25 (m, 1H), 3.18 (dd, 1H), 3.08 (m, 1H), 2.83-2.62 (m, 8H), 2.50 (s, 3H), 2.47-2.38 (m, 2H), 2.31 (m, 1H), 2.22 (m, 1H), 1.94 (s, 3H), 1.56 (m, 1H), 1.42 (m, 1H). UPLC-MS-2d: Rt = 3.85 min; MS m/z [M + H]⁺ 548.2/550.2. |
| | (3aS*,7aS*)-5-(1-(2- | | |
| | acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 2-methyloctahydro-1H- | | |
| | pyrrolo[3,4-c]pyridin-1- | | |
| | one | | |
| 18a/18b | 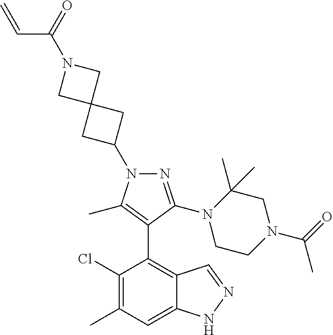 [SMILES: C=CC(=O)N1CC2(CC(n3nc(N4CCN(C(C)=O)CC4(C)C)c(-c4c(Cl)c(C)cc5[nH]ncc45)c3C)C2)C1] | Using Method-3 from Intermediate C10 (Step 3) and C-SFC-2 (mobile phase: CO₂/[IPA + 0.1% Et₃N] 65/35); Example 18a = 1^{st} eluting isomer, Example 18b = 2^{nd} eluting isomer | Example 18b: ¹H NMR (400 MHz, DMSO-d₆) δ 13.0 (s, 1H), 7.61 (s, 1H), 7.48 (s, 1H), 6.33 (m, 1H), 6.11 (m, 1H), 5.68 (m, 1H), 4.77 (m, 1H), 4.35 (s, 1H), 4.29 (s, 1H), 4.07 (s, 1H), 4.00 (s, 1H), 3.15 (m, 1H), 3.07-2.84 (m, 5H), 2.76-2.70 (m, 4H), 2.50 (s, 3H), 2.01 (s, 3H), 1.96 (s, 1.5H), 1.92 (s, 1.5H), 1.04-0.94 (m, 6H). UPLC-MS-2d: Rt = 4.25 min; MS m/z [M + H]⁺ 550.3/552.3; C-SFC- 3 (mobile phase: CO₂/[IPA + 0.1% Et3N] 65/35): Rt = 2.78 min, Example 18a: C-SFC-3 (mobile phase: CO₂/[IPA + 0.1% Et₃N] 65/35): Rt = 1.07 min. |
| | 1-(6-(3-(4-acetyl-2,2- | | |
| | dimethylpiperazin-1-yl)-4- | | |
| | (5-chloro-6-methyl-1H- | | |
| | indazol-4-yl)-5-methyl- | | |
| | 1H-pyrazol-1-yl)-2- | | |
| | azaspiro[3.3]heptan-2- | | |
| | yl)prop-2-en-1-one | | |
| 19a/19b | 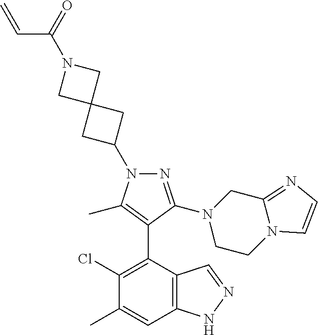 [SMILES: C=CC(=O)N1CC2(CC(n3nc(N4CCn5ccnc5C4)c(-c4c(Cl)c(C)cc5[nH]ncc45)c3C)C2)C1] | Using Method-3 from 5,6,7,8- tetrahydroimidazo [1,2-a]pyrazine (Step 1) and C- HPLC-4 (mobile phase: n- heptane/CH₂Cl₂/ EtOH/Et₃N 60/20/20/0.05%); Example 19a = 1^{st} eluting isomer, Example 19b = 2^{nd} eluting isomer | Example 19a: ¹H NMR (600 MHz, DMSO-d₆) δ δ 13.15 (s, 1H), 7.65 (s, 1H), 7.54 (s, 1H), 6.97 (s, 1H), 6.79 (s, 1H), 6.39- 6.25 (m, 1H), 6.17-6.07 (m, 1H), 5.73-5.64 (m, 1H), 4.79-4.68 (m, 1H), 4.35 (s, 1H), 4.29 (s, 1H), 4.16-4.07 (m, 2H), 4.05 (s, 1H), 4.01 (s, 1H), 3.84-3.74 (m, 1H), 3.66-3.57 (m, 1H), 3.29-3.12 (m, 2H), 2.50 (s, 3H), 2.81-2.65 (m, 4H), 1.96 (s, 3H). UPLC-MS-2f: Rt = 0.66 min; MS m/z [M + H]⁺ 517.2/519.2; C-HPLC-6 (mobile phase: heptane/CH₂Cl₂/EtOH/Eₜ₃N 55/20/25/0.05%): Rt = 1.16 min, Example 19b: C-HPLC-6 (mobile phase: heptane/CH₂Cl₂/EtOH/Et₃N 55/20/25/0.05%): Rt = 2.18 min. |
| | 1-(6-(4-(5-chloro-6- | | |
| | methyl-1H-indazol-4-yl)- | | |
| | 3-(5,6- | | |
| | dihydroimidazo[1,2- | | |
| | a]pyrazin-7(8H)-yl)-5- | | |
| | methyl-1H-pyrazol-1-yl)- | | |
| | 2-azaspiro[3.3]heptan-2- | | |
| | yl)prop-2-en-1-one | | |
| 20a/20b | 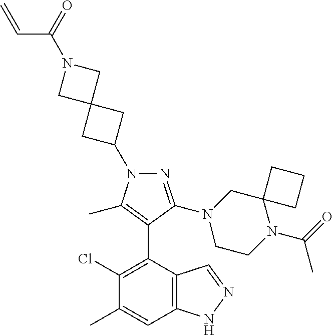 [SMILES: C=CC(=O)N1CC2(CC(n3nc(N4CCN(C(C)=O)C5(CCC5)C4)c(-c4c(Cl)c(C)cc5[nH]ncc45)c3C)C2)C1] | Using Method-3 from Intermediate A17 (Step 1) and C-SFC-36 (mobile phase: CO₂/MeOH 60/40); Example 20a = 1^{st} eluting isomer, Example 20b = 2^{nd} eluting isomer | Example 20a: ¹H NMR (600 MHz, DMSO-d₆) δ 13.1 (s, 1H), 7.63 (s, 1H), 7.52 (s, 1H), 6.38- 6.27 (m, 1H), 6.15-6.07 (m, 1H), 5.72-5.63 (m, 1H), 4.78-4.67 (m, 1H), 4.35 (s, 1H), 4.28 (s, 1H), 4.06 (s, 1H), 3.99 (s, 1H), 3.27- 3.13 (m, 2H), 2.94-2.85 (m, 1H), 2.82-2.71 (m, 3H), 2.70-2.58 (m, 5H), 2.49 (s, 3H), 2.12-2.02 (m, 1H), 1.96 (s, 3H), 1.94-1.90 (m, 1H), 1.88 (s, 3H), 1.57-1.45 (m, 1H), 1.39-1.11 (m, 2H); UPLC-MS-2b: Rt = 0.95 min; MS m/z [M + H]⁺ 562.3/564.3; C-SFC- 37 (mobile phase: CO₂/MeOH 60/40): Rt = 2.65 min, Example 20b: C-SFC-37 (mobile phase: CO₂/MeOH 60/40): Rt = 3.27 min. |
| | 1-(6-(3-(5-acetyl-5,8- | | |
| | diazaspiro[3.5]nonan-8- | | |
| | yl)-4-(5-chloro-6-methyl- | | |
| | 1H-indazol-4-yl)-5- | | |
| | methyl-1H-pyrazol-1-yl)- | | |
| | 2-azaspiro[3.3]heptan-2- | | |
| | yl)prop-2-en-1-one | | |
| 21a/21b | 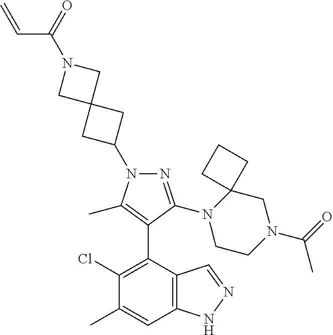 [SMILES: C=CC(=O)N1CC2(CC(n3nc(N4CCN(C(C)=O)CC45CCC5)c(-c4c(Cl)c(C)cc5[nH]ncc45)c3C)C2)C1] | Using Method-3 from Intermediate A18 and C-SFC-2 (mobile phase: CO₂/IPA 65/35); Example 21a = 1^{st} eluting isomer, Example 21b = 2^{nd} eluting isomer | Example 21b: ¹H NMR (600 MHz, DMSO-d₆) 13.0 (s, 1H), 7.62 (s, 1H), 7.47 (s, 1H), 6.39- 6.25 (m, 1H), 6.15-6.03 (m, 1H), 5.72-5.63 (m, 1H), 4.82-4.68 (m, 1H), 4.34 (s, 1H), 4.28 (s, 1H), 4.06 (s, 1H), 3.99 (s, 1H), 3.84- 3.68 (m, 0.5H), 3.55-3.41 (m, 1H), 3.37-3.34 (m, 0.5H), 3.29- 3.18 (m, 1H), 3.08-3.02 (m, 0.5H), 2.93-2.88 (m, 0.5H), 2.79- 2.57 (m, 6H), 2.48 (s, 3H), 2.16- 2.05 (m, 1H), 1.99 (s, 3H), 1.98 (s, 1.5H), 1.93 (s, 1.5H), 1.95- 1.90 (m, 0.5H), 1.75-1.68 (m, 1H), 1.67-1.56 (m, 3.5H). UPLC- MS-2b: Rt = 0.95 min; MS m/z [M + H]⁺ 562.2/ 564.2; C-SFC-3 (mobile phase: CO₂/IPA 65/35): Rt = 2.85 min, Example 21a: C- SFC-3 (mobile phase: CO₂/IPA 65/35): Rt = 1.37 min. |
| | 1-(6-(3-(8-acetyl-5,8- | | |
| | diazaspiro[3.5]nonan-5- | | |
| | yl)-4-(5-chloro-6-methyl- | | |
| | 1H-indazol-4-yl)-5- | | |
| | methyl-1H-pyrazol-1-yl)- | | |
| | 2-azaspiro[3.3]heptan-2- | | |
| | yl)prop-2-en-1-one | | |
| 22a/22b | 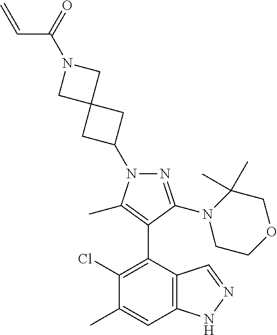 [SMILES: C=CC(=O)N1CC2(CC(n3nc(N4CCOCC4(C)C)c(-c4c(Cl)c(C)cc5[nH]ncc45)c3C)C2)C1] | Using Method-3 from 3,3- dimethyl- morpholine and C-SFC-2 (mobile phase: CO₂/IPA 73/27); Example 22a = 1^{st} eluting isomer, Example 22b = 2^{nd} eluting isomer | Example 22b: ¹H NMR (600 MHz, DMSO-d₆) δ 13.01 (s, 1H), 7.60 (s, 1H), 7.46 (s, 1H), 6.37- 6.25 (m, 1H), 6.15-6.06 (m, 1H), 5.72-5.64 (m, 1H), 4.81-4.71 (m, 1H), 4.35 (s, 1H), 4.30 (s, 1H), 4.06 (s, 1H), 4.01 (s, 1H), 3.32- 3.26 (m, 1H), 3.22-3.14 (m, 1H), 3.09-3.02 (m, 2H), 2.86-2.80 (m, 2H), 2.77-2.69 (m, 4H), 2.48 (s, 3H), 1.99 (s, 3H), 1.08 (m, 3H), 1.01 (m, 3H). UPLC-MS-2b: Rt = 1.02 min; MS m/z [M + H]⁺ 509.2/ 511.2; C-SFC-3 (mobile phase: CO₂/IPA 73/27): Rt = 2.67 min, Example 22a: C-SFC-3 (mobile phase: CO₂/IPA 73/27): Rt = 1.81 min. |
| | 1-(6-(4-(5-chloro-6- | | |
| | methyl-1H-indazol-4-yl)- | | |
| | 3-(3,3- | | |
| | dimethylmorpholino)-5- | | |
| | methyl-1H-pyrazol-1-yl)- | | |
| | 2-azaspiro[3.3]heptan-2- | | |
| | yl)prop-2-en-1-one | | |
| 23a/23b | 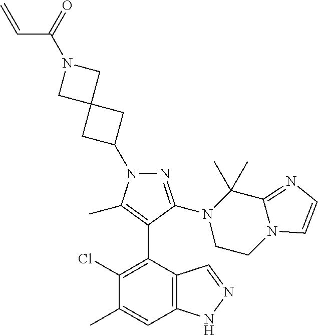 [SMILES: C=CC(=O)N1CC2(CC(n3nc(N4CCn5ccnc5C4(C)C)c(-c4c(Cl)c(C)cc5[nH]ncc45)c3C)C2)C1] | Using Method-3 from 8,8-dimethyl- 5,6,7,8- tetrahydroimidazo [1,2-a]pyrazine A61 (Step 1) and C-SFC-2 (mobile phase: CO₂/[IPA + 0.025% NH₃] 65/35); Example 23a = 1^{st} eluting isomer, Example 23b = 2^{nd} eluting isomer | Example 23b: ¹H NMR (400 MHz, DMSO-d₆) δ 13.04 (s, 1H), 7.64 (s, 1H), 7.47 (s, 1H), 6.83 (m, 1H), 6.72 (m, 1H), 6.38-6.25 (m, 1H), 6.16-6.06 (m, 1H), 5.73- 5.64 (m, 1H), 4.86-4.72 (m, 1H), 4.36 (s, 1H), 4.29 (s, 1H), 4.07 (s, 1H), 4.01 (s, 1H), 3.66-3.55 (m, 1H), 3.54-3.45 (m, 1H), 3.43- 3.36 (m, 2H), 2.82-2.71 (m, 4H), 2.48 (s, 3H), 2.03 (s, 3H), 1.39 (br. s, 3H), 1.32 (br. s, 3H). UPLC-MS-2a: Rt = 0.77 min; MS m/z [M + H]⁺ 545.2/547.2; C-SFC- 3 (mobile phase: CO₂/[IPA + 0.1%Et₃N] 65/35): Rt = 2.77 min, Example 23a: C- SFC-3 (mobile phase: CO₂/[IPA + 0.1%Et₃N] 65/35): Rt = 2.02 min. |
| | 1-(6-(4-(5-chloro-6- | | |
| | methyl-1H-indazol-4-yl)- | | |
| | 3-(8,8-dimethyl-5,6- | | |
| | dihydroimidazo[1,2- | | |
| | a]pyrazin-7(8H)-yl)-5- | | |
| | methyl-1H-pyrazol-1-yl)- | | |
| | 2-azaspiro[3.3]heptan-2- | | |
| | yl)prop-2-en-1-one | | |
| 24a/24b | 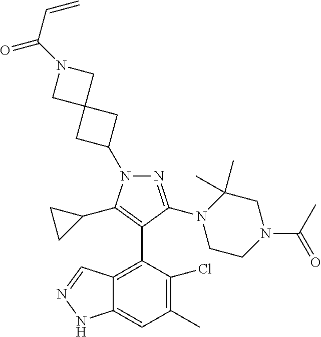 [SMILES: C=CC(=O)N1CC2(CC(n3nc(N4CCN(C(C)=O)CC4(C)C)c(-c4c(Cl)c(C)cc5[nH]ncc45)c3C3CC3)C2)C1] | Using Method-3 from Intermediate C18 (Step2) and C-SFC-1 (mobile phase: CO₂/IPA 67/33); Example 24a = 1^{st} eluting isomer, Example 24b = 2^{nd} eluting isomer | Example 24b: ¹H NMR (400 MHz, DMSO-d₆) (mixture of rotamers) δ 12.99 (s, 1H), 7.59 (s, 1H), 7.47 (s, 1H), 6.41-6.23 (m, 1H), 6.16-6.05 (m, 1H), 5.73- 5.63 (m, 1H), 5.09-4.95 (m, 1H), 4.37 (s, 1H), 4.30 (s, 1H), 4.08 (s, 1H), 4.01 (s, 1H), 3.25-2.95 (m, 4H), 2.95-2.70 (m, 6H), 2.48 (s, 3H), 1.95 (s, 1.5H), 1.91 (s, 1.5H), 1.82-1.70 (m, 1H), 1.00 (s, 1.5H), 0.99 (s, 1.5H), 0.98 (s, 1.5H), 0.96 (s, 1.5H), 0.60-0.52 (m, 2H), 0.13-0.06 (m, 1H), 0.02- 0.01 (m, 1H); UPLC-MS-2b: Rt = 0.97 min; MS m/z [M + H]⁺ 576.3/ 578.3; C-SFC-3 (mobile phase: CO₂/IPA 67/33): Rt = 2.86 min, Example 24a: C-SFC-3 (mobile phase: CO₂/IPA 67/33): Rt = 1.40 min. |
| | 1-(6-(3-(4-acetyl-2,2- | | |
| | dimethylpiperazin-1-yl)-4- | | |
| | (5-chloro-6-methyl-1H- | | |
| | indazol-4-yl)-5- | | |
| | cyclopropyl-1H-pyrazol- | | |
| | 1-yl)-2- | | |
| | azaspiro[3.3]heptan-2- | | |
| | yl)prop-2-en-1-one | | |
| 25a/25b | 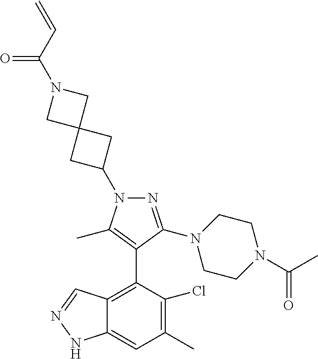 [SMILES: C=CC(=O)N1CC2(CC(n3nc(N4CCN(C(C)=O)CC4)c(-c4c(Cl)c(C)cc5[nH]ncc45)c3C)C2)C1] | Using Method-3 from Intermediate C19 (Step 3) and C-HPLC-25: (mobile phase: [hexane + 0.1% DEA]/[IPA-MeOH (50:50)] 60:40), flow rate 18 mL/min). Example 25a = 1^{st} eluting isomer, Example 25b = 2^{nd} eluting isomer | Example 25a: ¹H NMR (400 MHz, DMSO-d₆) δ 13.12 (s, 1H), 7.67 (s, 1H), 7.51 (s, 1H), 6.33 (m, 1H), 6.13 (m, 1H), 5.70 (m, 1H), 4.75 (m, 1H), 4.35 (s, 1H), 4.29 (s, 1H), 4.06 (s, 1H), 4.00 (s, 1H), 3.27-3.16 (m, 4H), 2.89- 2.68 (m, 8H), 2.50 (s, 3H), 1.94 (s, 3H), 1.90 (s, 3H). UPLC-MS- 5: Rt = 1.47 min; MS m/z [M + H]⁺ 522.9/524.9; C-HPLC-26 (mobile phase: hexane/[IPA- MeOH (50-50)] from 20 to 70%); Rt = 12.2 min, Example 25b: C- HPLC-26 (mobile phase: hexane/[IPA-MeOH (50-50)] from 20 to 70%); Rt = 13.8 min. |
| | 1-(6-(3-(4- | | |
| | acetylpiperazin-1-yl)-4- | | |
| | (5-chloro-6-methyl-1H- | | |
| | indazol-4-yl)-5-methyl- | | |
| | 1H-pyrazol-1-yl)-2- | | |
| | azaspiro[3.3]heptan-2- | | |
| | yl)prop-2-en-1-one | | |
| 26a/26b | 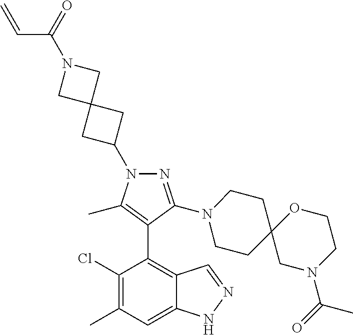 [SMILES: C=CC(=O)N1CC2(CC(n3nc(N4CCC5(CC4)CN(C(C)=O)CCO5)c(-c4c(Cl)c(C)cc5[nH]ncc45)c3C)C2)C1] | Using Method-3e from Intermediate A65 (Step 1) and C-HPLC-30 (mobile phase: hexane/IPA 52:48). Example 26a = 1^{st} eluting isomer, Example 26b = 2^{nd} eluting isomer | Example 26b: ¹H NMR (400 MHz, DMSO-d₆) δ 13.04 (s, 1H), 7.62 (s, 1H), 7.46 (s, 1H), 6.34 (m, 1H), 6.12 (m, 1H), 5.68 (m, 1H), 4.80 (m, 1H), 4.69 (s, 1H), 4.63 (s, 1H), 4.33 (s, 1H), 4.27 (s, 1H), 4.04 (s, 1H), 3.98 (s, 1H), 3.52-3.48 (m, 2H), 3.30 (m, 2H), 3.23-3.18 (m, 2H), 2.82-2.59 (m, 5H), 2.50 (s, 3H), 2.32 (m, 1H), 1.95-1.89 (m, 6H), 1.53-1.23 (m, 4H); UPLC-MS-5: Rt = 1.49 min; MS m/z [M + H]⁺ 592.5/594.5; C- HPLC-31 (mobile phase: hexane/IPA 50:50): Rt = 17.5 min, Example 26a: C-HPLC-31 (mobile phase: hexane/IPA 50:50): Rt = 13.0 min. |
| | 1-(6-(3-(4-acetyl-1-oxa- | | |
| | 4,9- | | |
| | diazaspiro [5.5]undecan- | | |
| | 9-yl)-4-(5-chloro-6- | | |
| | methyl-1H-indazol-4-yl)- | | |
| | 5-methyl-1H-pyrazol-1- | | |
| | yl)-2- | | |
| | azaspiro[3.3]heptan-2- | | |
| | yl)prop-2-en-1-one | | |
| 27a/27b | 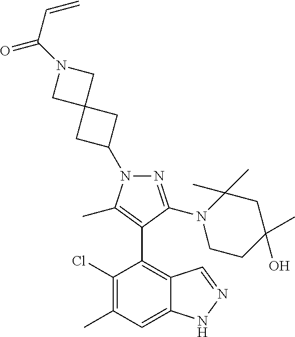 [SMILES: C=CC(=O)N1CC2(CC(n3nc(N4CCC(C)(O)CC4(C)C)c(-c4c(Cl)c(C)cc5[nH]ncc45)c3C)C2)C1] | Using Method-3c starting from Intermediate C108a (Step 2) and RP-HPLC-5: (mobile phase: Water, 5 mM ammonium carbonate, 0.025% NH₃/ CH₃CN; 65/35) Example 27a = 1^{st} eluting isomer, Example 27b = 2^{nd} eluting isomer | Example 27b: ¹H NMR (400 MHz, Methanol-d₄) δ 7.62 (s, 1H), 7.45 (s, 1H), 6.42 (m, 1H), 6.37 (m, 1H), 5.78 (m, 1H), 4.80 (m, 1H), 4.46 (s, 1H), 4.44 (s, 1H), 4.22 (s, 1H), 4.19 (s, 1H), 3.58 (m, 1H), 2.93-2.68 (m, 5H), 2.55 (s, 3H), 2.08 (s, 3H), 1.45- 1.39 (m, 3H), 1.36 (s, 3H), 1.15 (m, 1H), 1.10 (s, 3H), 0.82 (s, 1.5H), 0.80 (s, 1.5H). UPLC-MS- 5: Rt = 1.60 min; MS m/z [M + H]⁺ 537.6/539.5; RP-HPLC-7 (mobile phase: [water + 0.025% NH₃]/[CH₃CN + 0.025%NH₃] 60/40 to 90/10): Rt = 13.5 min, Example 27a: RP-HPLC-7 (mobile phase: [water + 0.025% NH₃]/[CH₃CN + 0.025%NH₃] 60/40 to 90/10): Rt = 11.2 min. |
| | 1-(6-(4-(5-chloro-6- | | |
| | methyl-1H-indazol-4-yl)- | | |
| | 3-(4-hydroxy-2,2,4- | | |
| | trimethylpiperidin-1-yl)-5- | | |
| | methyl-1H-pyrazol-1-yl)- | | |
| | 2-azaspiro[3.3]heptan-2- | | |
| | yl)prop-2-en-1-one | | |
| 28a/28b | 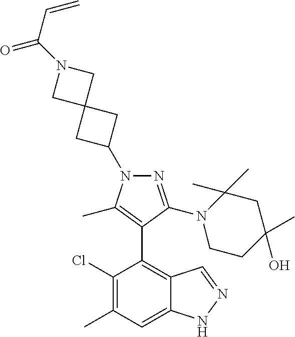 [SMILES: C=CC(=O)N1CC2(CC(n3nc(N4CCC(C)(O)CC4(C)C)c(-c4c(Cl)c(C)cc5[nH]ncc45)c3C)C2)C1] | Using Method-3c from Intermediate C108b (Step 2) and RP-HPLC-5: (mobile phase: Water, 5 mM ammonium carbonate, 0.025% NH₃/ CH₃CN; 65/35); Example 28a = 1^{st} eluting isomer, Example 28b = 2^{nd} eluting isomer | Example 28a: ¹H NMR (400 MHz, Methanol-d₄) δ 7.67 (s, 1H), 7.45 (s, 1H), 6.41 (m, 1H), 6.38 (m, 1H), 5.78 (m, 1H), 4.80 (m, 1H), 4.46 (m, 1H), 4.43 (s, 1H), 4.22 (m, 2H), 4.18 (s, 1H), 3.27 (m, 1H), 2.94-2.68 (m, 5H), 2.57 (s, 3H), 2.05 (s, 3H), 1.50- 1.20 (m, 6H), 1.10-0.95 (m, 7H); UPLC-MS-5: Rt = 1.52 min; MS m/z [M + H]⁺ 537.6/539.6; RP- HPLC-7 (mobile phase: [water + 0.025% NH₃]/ [CH₃CN + 0.025%NH₃] 60/40 to 90/10): Rt = 10.8 min, Example 28b: RP-HPLC-7 (mobile phase: [water + 0.025% NH₃]/ [CH₃CN + 0.025%NH₃] 60/40 to 90/10): Rt = 13.5 min. |
| | 1-(6-(4-(5-chloro-6- | | |
| | methyl-1H-indazol-4-yl)- | | |
| | 3-(4-hydroxy-2,2,4- | | |
| | trimethylpiperidin-1-yl)-5- | | |
| | methyl-1H-pyrazol-1-yl)- | | |
| | 2-azaspiro[3.3]heptan-2- | | |
| | yl)prop-2-en-1-one | | |
| 29a/29b | 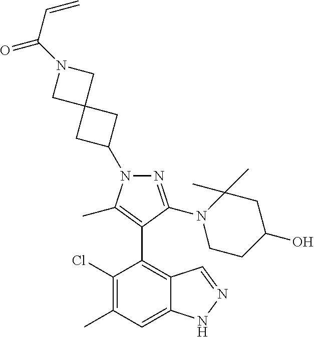 [SMILES: C=CC(=O)N1CC2(CC(n3nc(N4CCC(O)CC4(C)C)c(-c4c(Cl)c(C)cc5[nH]ncc45)c3C)C2)C1] | Using Method-3c from Intermediate C76a (Step 2) and RP-HPLC-6: (mobile phase: Water, 5 mM ammonium carbonate, 0.025% NH₃/ CH₃CN; 80/20 to 55/45 in 30 min); Example 29a = 1^{st} eluting isomer, Example 29b = 2^{nd} eluting isomer | Example 29b: ¹H NMR (400 MHz, Methanol-d₄) δ 7.61 (m, 1H), 7.44 (m, 1H), 6.40-6.36 (m, 1H), 6.34-6.24 (m, 1H), 5.77- 5.74 (m, 1H), 4.85-4.78 (m, 1H), 4.44 (s, 1H), 4.41 (s, 1H), 4.21 (s, 1H), 4.18 (s, 1H), 3.80-3.75 (m, 1H), 3.02-2.81 (m, 6H), 2.54 (s, 3H), 2.08 (s, 3H), 1.79-1.72 (m, 1H), 1.52-1.48 (m, 1H), 1.28- 1.19 (m, 4H), 1.10-0.98 (m, 1H), 0.75 (s, 3H); UPLC-MS-5: Rt = 1.45 min; MS m/z [M + H]⁺ 523.5/ 525.5; RP-HPLC-10: Rt = 5.72 min, Example 29a: RP-HPLC- 10: Rt = 5.41 min. |
| | 1-(6-(4-(5-chloro-6- | | |
| | methyl-1H-indazol-4-yl)- | | |
| | 3-(4-hydroxy-2,2- | | |
| | dimethylpiperidin-1-yl)-5- | | |
| | methyl-1H-pyrazol-1-yl)- | | |
| | 2-azaspiro[3.3]heptan-2- | | |
| | yl)prop-2-en-1-one | | |
| 30a/30b | 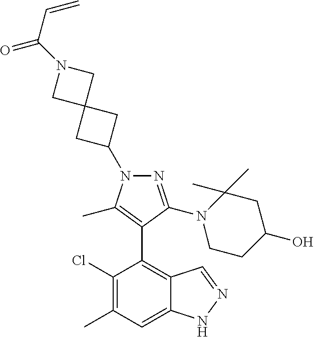 [SMILES: C=CC(=O)N1CC2(CC(n3nc(N4CCC(O)CC4(C)C)c(-c4c(Cl)c(C)cc5[nH]ncc45)c3C)C2)C1] | Using Method-3c from Intermediate C76b (Step 2) and RP-HPLC-6: (mobile phase: Water, 5 mM ammonium carbonate, 0.1% NH₃/CH₃CN; 80/20 to 55/45 in 30 min); Example 30a = 1^{st} eluting isomer, Example 30b = 2^{nd} eluting isomer | Example 30a: ¹H NMR (400 MHz, Methanol-d₄) δ 7.65 (s, 1H), 7.45 (s, 1H), 6.36 (m, 1H), 6.26 (d, 1H), 5.77 (d, 1H), 4.78- 4.71 (m, 1H), 4.45 (s, 1H), 4.41 (s, 1H), 4.21 (s, 1H), 4.17 (s, 1H), 3.73-3.71 (m, 1H), 3.06-3.02 (m, 2H), 2.99-2.81 (m, 4H), 2.56 (s, 3H), 2.03 (s, 3H), 1.65-1.56 (m, 2H), 1.31 (s, 3H), 1.21-1.18 (m, 4H), 0.80-0.92 (m, 1H); UPLC- MS-5: Rt = 1.39 min; MS m/z [M + H]⁺ 523.5/525.5; RP-HPLC- 10: Rt = 5.42 min, Example 30b: RP-HPLC-10: Rt = 5.73 min. |
| | 1-(6-(4-(5-chloro-6- | | |
| | methyl-1H-indazol-4-yl)- | | |
| | 3-(4-hydroxy-2,2- | | |
| | dimethylpiperidin-1-yl)-5- | | |
| | methyl-1H-pyrazol-1-yl)- | | |
| | 2-azaspiro[3.3]heptan-2- | | |
| | yl)prop-2-en-1-one | | |
| 31a/31b | 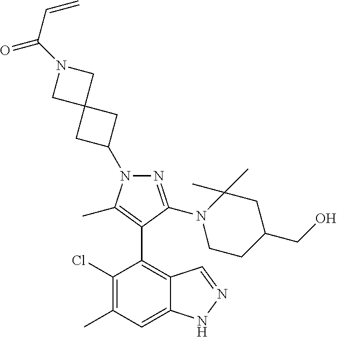 [SMILES: C=CC(=O)N1CC2(CC(n3nc(N4CCC(CO)CC4(C)C)c(-c4c(Cl)c(C)cc5[nH]ncc45)c3C)C2)C1] | Using Method-3c from C114a (Step 2) and RP-HPLC- 5: (mobile phase: Water, 5 mM ammonium carbonate, 0.1% NH₃/CH₃CN; 60/40) Example 31a = 1^{st} eluting isomer, Example 31b = 2^{nd} eluting isomer | Example 31b: ¹H NMR (400 MHz, Methanol-d₄) δ 7.60 (s, 1H), 7.44 (s, 1H), 6.44 (m, 1H), 6.32 (m, 1H), 5.77 (d, 1H), 4.80 (m, 1H), 4.44 (s, 1H), 4.42 (s, 1H), 4.21 (s, 1H), 4.19 (s, 1H), 3.22 (d, 2H), 3.00-2.81 (m, 6H), 2.54 (s, 3H), 2.08 (s, 3H), 1.77 (m, 1H), 1.65 (d, 1H), 1.41-1.28 (m, 2H), 1.19 (s, 3H), 0.9 (m, 1H), 0.73 (s, 3H); UPLC-MS-5: Rt = 1.50 min; MS m/z [M + H]⁺ 537.9/ 539.9; RP-HPLC-9 (mobile phase: [water + 0.025% NH₃]/[CH₃CN + 0.025% NH₃] 60/40): Rt = 8.56 min. Example 31a: RP-HPLC-9 (mobile phase: (mobile phase: [water + 0.025% NH₃]/[CH₃CN + 0.025% NH₃] 60/40): Rt = 6.85 min. |
| | 1-(6-(4-(5-chloro-6- | | |
| | methyl-1H-indazol-4-yl)- | | |
| | 3-(4-(hydroxymethyl)-2,2- | | |
| | dimethylpiperidin-1-yl)-5- | | |
| | methyl-1H-pyrazol-1-yl)- | | |
| | 2-azaspiro[3.3]heptan-2- | | |
| | yl)prop-2-en-1-one | | |
| 32a/32b | 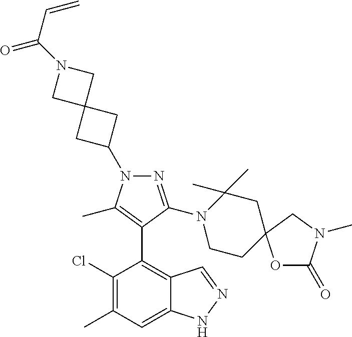 [SMILES: C=CC(=O)N1CC2(CC(n3nc(N4CCC5(CN(C)C(=O)O5)CC4(C)C)c(-c4c(Cl)c(C)cc5[nH]ncc45)c3C)C2)C1] | Using Method-3c from Intermediate C96a (Step 3) RP- HPLC-5 (mobile phase: Water, 5 mM ammonium carbonate, 0.1% NH₃/CH₃CN; 65/35); Example 32a = 1^{st} eluting isomer, Example 32b = 2^{nd} eluting isomer | Example 32b: ¹H NMR (400 MHz, Methanol-d₄) δ 7.62 (s, 1H), 7.45 (s, 1H), 6.41-6.34 (m, 1H), 6.28-6.24 (m, 1H), 5.75 (d, 1H), 4.90 (m, 1H), 4.45 (s, 1H), 4.42 (s, 1H), 4.21 (s, 1H), 4.18 (s, 1H), 3.50 (m, 1H), 3.33-3.23 (m, 2H), 3.04-2.90 (m, 3H), 2.90- 2.80 (m, 5H), 2.55 (s, 3H), 2.04 (s, 3H), 1.68-1.45 (m, 4H), 1.25 (s, 3H), 0.91 (s, 1.5H), 0.89 (s, 1.5H); UPLC-MS-5: Rt = 1.62 min; MS m/z [M + H]⁺ 593/595; RP-HPLC-7 (mobile phase: [water + 0.025% NH₃]/[CH₃CN + 0.025% NH3] 60/40 to 10/90): Rt = 13.2 min. Example 32a: RP- HPLC-7 (mobile phase: A:[water + 0.025% NH₃]/B:[CH₃CN + 0.025% NH₃] 40% B for 15 min then 90%): Rt = 10.9 min. |
| | 8-(1-(2-acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 3,7,7-trimethyl-1-oxa-3,8- | | |
| | diazaspiro[4.5]decan-2- | | |
| | one | | |
| 33a/33b | 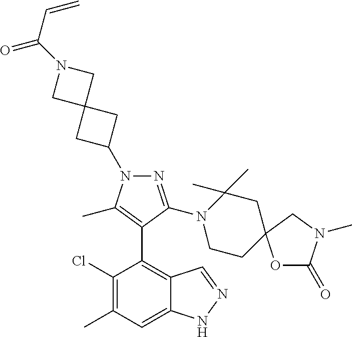 [SMILES: C=CC(=O)N1CC2(CC(n3nc(N4CCC5(CN(C)C(=O)O5)CC4(C)C)c(-c4c(Cl)c(C)cc5[nH]ncc45)c3C)C2)C1] | Using Method-3c from Intermediate C96b (Step 3) RP- HPLC-5 (mobile phase: Water, 5 mM ammonium carbonate, 0.025% NH₃/ CH₃CN; 65/35); Example 33a = 1^{st} eluting isomer, Example 33b = 2^{nd} eluting isomer | Example 33a: ¹H NMR (400 MHz, Methanol-d₄) δ 7.63 (s, 1H), 7.45 (s, 1H), 6.37 (dd, 1H), 6.26 (d, 1H), 5.76 (d, 1H), 4.81 (m, 1H), 4.44 (s, 1H), 4.41 (s, 1H), 4.21 (s, 1H), 4.17 (s, 1H), 3.26 (m, 2H), 2.97-2.91 (m, 4H), 2.89-2.83 (m, 2H), 2.80 (s, 3H), 2.55 (s, 3H), 2.04 (s, 3H), 1.73- 1.35 (m, 4H), 1.32 (s, 1.5H), 1.30 (s, 1.5H), 1.08 (s, 1.5H), 1.06 (s, 1.5H); UPLC-MS-5: Rt = 1.58 min; MS m/z [M + H]⁺ 592.9/ 594.9; RP-HPLC-7 (mobile phase: [water + 0.025% NH₃]/[CH₃CN + 0.025% NH₃] 60/40 to 10/90): Rt = 12.2 min. Example 33b: RP-HPLC-7 (mobile phase: A:[water + 0.025% NH₃]/B:[CH₃CN + 0.025% NH₃] 40% B for 15 min then 90%): Rt = 14.2 min. |
| | 8-(1-(2-acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 3,7,7-trimethyl-1-oxa-3,8- | | |
| | diazaspiro[4.5]decan-2- | | |
| | one | | |
| 34a/34b | 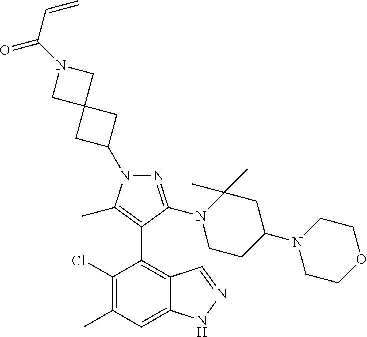 [SMILES: C=CC(=O)N1CC2(CC(n3nc(N4CCC(N5CCOCC5)CC4(C)C)c(-c4c(Cl)c(C)cc5[nH]ncc45)c3C)C2)C1] | Using Method-3 from Intermediate C94a (Step 2) and RP-HPLC-5 (mobile phase: A: [5 mM ammonium bicarbonate + 0.1% NH₃ in water]/B: CH₃CN, gradient: 40% B for 45 min, 100% B for 2 min, 100% to 40% B in 5 min). Example 34a = 1^{st} eluting isomer, Example 34b = 2^{nd} eluting isomer | Example 34b: ¹H NMR (400 MHz, Methanol-d₄) δ 7.60 (s, 1H), 7.44 (s, 1H), 6.41-6.34 (m, 1H), 6.28-6.24 (d, 1H), 5.75 (d, 1H), 4.85 (m, 1H), 4.45 (s, 1H), 4.42 (s, 1H), 4.22 (s, 1H), 4.19 (s, 1H), 3.60 (m, 4H), 3.50 (m, 1H), 3.07 (m, 1H), 2.95-2.82 (m, 4H), 2.54 (s, 3H), 2.49 (m, 5H), 2.08 (s, 3H), 1.86 (m, 1H), 1.60 (d, 1H), 1.20 (s, 3H), 1.19 (m,1H), 0.95 (m, 1H), 0.76 (s, 3H); UPLC- MS-5: Rt = 1.53 min, MS m/z [M + H]⁺ 592.6/594.6; RP-HPLC- 7 (mobile phase: A: [water + 0.1% NH₃]/B: [CH₃CN + 0.1% NH₃], gradient: 40% B for 15 min, 40 to 90% in 0.01 min, then 90% for 5 min): Rt = 8.09 min. Example 34a: RP-HPLC-7 (mobile phase: A: [water + 0.1% NH₃]/B: [CH₃CN + 0.1% NH₃], |
| | | | gradient: 40% B for 15 min, 40 to |
| | 1-(6-(4-(5-chloro-6- | | 90% in 0.01 min, then 90% for 5 |
| | methyl-1H-indazol-4-yl)- | | min): Rt = 7.73 min. |
| | 3-(2,2-dimethyl-4- | | |
| | morpholinopiperidin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 35a/35b | 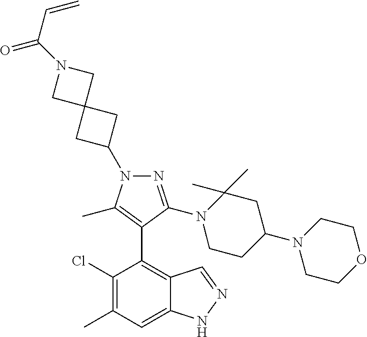 [SMILES: C=CC(=O)N1CC2(CC(n3nc(N4CCC(N5CCOCC5)CC4(C)C)c(-c4c(Cl)c(C)cc5[nH]ncc45)c3C)C2)C1] | Using Method-3 from C94b (Step 2) and RP-HPLC- 5: (mobile phase: A: [5 mM ammonium bicarbonate + 0.1% NH₃ in water]/B: CH₃CN gradient: 35% B for 48 min, 100% B for 2 min then 100 to 35% B in 5 min); Example 35a = 1^{st} eluting isomer, Example 35b = 2^{nd} eluting isomer | Example 35a: ¹H NMR (400 MHz, Methanol-d₄) δ 7.65 (s, 1H), 7.45 (s, 1H), 6.39-6.34 (m, 1H), 6.29-6.24 (d, 1H), 5.78- 5.76 (d, 1H), 4.81 (m, 1H), 4.45 (s, 1H), 4.41 (s, 1H), 4.22 (s, 1H), 4.18 (s, 1H), 3.64 (m, 4H), 3.07 (m, 1H), 2.91-2.81 (m, 5H), 2.55 (s, 3H), 2.47-2.43 (m, 5H), 2.03 (s, 3H), 1.69-1.62 (m, 2H), 1.36 (s, 3H), 1.18 (m, 1H), 1.15 (s, 3H), 0.86 (m, 1H); UPLC-MS-5: Rt = 1.50 min, MS m/z [M + H]⁺ 592.6/ 594.6; RP-HPLC-7 (mobile phase: A: [water + 0.1% NH₃]/B: [CH₃CN + 0.1% NH₃], gradient: 40% B for 15 min, 40 to 90% in 0.01 min, then 90% for 5 min): Rt = 7.73 min. Example 35b: RP-HPLC-7 (mobile phase: A: [water + 0.1% NH₃]/ B: |
| | | | [CH₃CN + 0.1% NH₃], gradient: |
| | 1-(6-(4-(5-chloro-6- | | 40% B for 15 min, 40 to 90% in |
| | methyl-1H-indazol-4-yl)- | | 0.01 min, then 90% for 5 min): |
| | 3-(2,2-dimethyl-4- | | Rt = 8.09 min. |
| | morpholinopiperidin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 36a/36b | 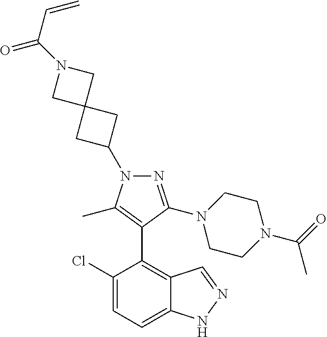 [SMILES: C=CC(=O)N1CC2(CC(n3nc(N4CCN(C(C)=O)CC4)c(-c4c(Cl)ccc5[nH]ncc45)c3C)C2)C1] | Using Method-3d starting from Intermediate C12 and D7 (Step 3) and C-HPLC-8 (mobile phase: n- heptane/IPA/Et₃N 85/15/0.05%); Example 36a = 1^{st} eluting isomer, Example 36b = 2^{nd} eluting isomer | Example 36b: ¹H NMR (400 MHz, DMSO-d₆) δ 13.2 (s, 1H), 7.74 (s, 1H), 7.54 (d, 1H), 7.46 (d, 1H), 6.31 (m, 1H), 6.11 (m, 1H), 5.68 (m, 1H), 4.72 (m, 1H), 4.35 (s, 1H), 4.29 (s, 1H), 4.06 (s, 1H), 4.00 (s, 1H), 3.27 (m, 3H), 3.18 (m, 1H), 2.90-2.81 (m, 2H), 2.78-2.65 (m, 6H), 1.96 (s, 3H), 1.90 (s, 3H); UPLC-MS-2d: Rt = 3.47 min; MS m/z [M + H]⁺ 508.1/510.1; C-HPLC-9 (mobile phase: heptane/IPA 80/20): Rt = 11.7 min, Example 36a: C- HPLC-9 (mobile phase: heptaneCO₂/IPA 80/20): Rt = 9.30 min. |
| | 1-(6-(3-(4- | | |
| | acetylpiperazin-1-yl)-4- | | |
| | (5-chloro-1H-indazol-4- | | |
| | yl)-5-methyl-1H-pyrazol- | | |
| | 1-yl)-2- | | |
| | azaspiro[3.3]heptan-2- | | |
| | yl)prop-2-en-1-one | | |
| 37a/37b | 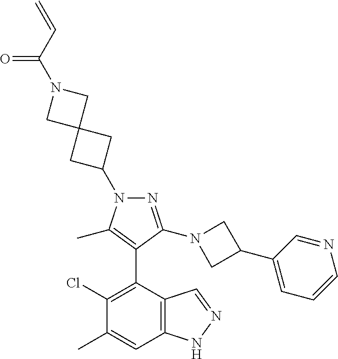 [SMILES: C=CC(=O)N1CC2(CC(n3nc(N4CC(c5cccnc5)C4)c(-c4c(Cl)c(C)cc5[nH]ncc45)c3C)C2)C1] | Using Method-3f from 3-(azetidin-3- yl)pyridine trifluoroacetate salt (Step 1) and C-SFC-4 (mobile phase: 35:65 [MeOH + 0.1% Et₃N]/CO₂); Example 37a = 1^{st} eluting isomer, Example 37b = 2^{nd} eluting isomer | Example 37b: ¹H NMR (600 MHz, DMSO-d₆) δ 13.1 (s, 1H), 8.39 (s, 2H), 7.69 (m, 2H), 7.45 (s, 1H), 7.30 (m, 1H), 6.30 (m, 1H), 6.09 (m, 1H), 5.66 (m, 1H), 4.71 (m, 1H), 4.35 (s, 1H), 4.28 (s, 1H), 4.05 (s, 1H), 3.98 (s, 1H), 3.85 (m, 1H), 3.64-3.75 (m, 2H), 3.42 (m, 1H), 3.26 (m, 1H), 2.76 (m, 2H), 2.66 (m, 2H), 2.46 (s, 3H), 1.97 (s, 3H); UPLC-MS-1a: Rt = 0.83 min, MS m/z [M + H]⁺ 528.6/530.6; C-SFC-3 (mobile phase: 35:65 [MeOH + 0.025% NH₃]/CO₂): Rt = 2.06 min. Example 37a: C-SFC-3 (mobile phase: 35:65 [MeOH + 0.025% NH₃]/CO₂): Rt = 1.21 min. |
| | 1-(6-(4-(5-chloro-6- | | |
| | methyl-1H-indazol-4-yl)- | | |
| | 5-methyl-3-(3-(pyridin-3- | | |
| | yl)azetidin-1-yl)-1H- | | |
| | pyrazol-1-yl)-2- | | |
| | azaspiro[3.3]heptan-2- | | |
| | yl)prop-2-en-1-one | | |
| 38a/38b | 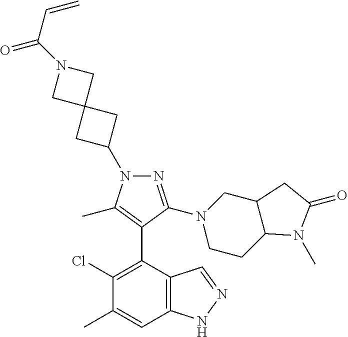 [SMILES: C=CC(=O)N1CC2(CC(n3nc(N4CCC5C(CC(=O)N5C)C4)c(-c4c(Cl)c(C)cc5[nH]ncc45)c3C)C2)C1] | Using Method-3 from Intermediate C26b (Step 3) and C-SFC-38 (mobile phase: CO₂/CH₃OH 60/40); Example 38a = 1^{st} eluting isomer, Example 38b = 2^{nd} eluting isomer | Example 38b: ¹H NMR (400 MHz, DMSO-d₆) δ 13.07 (s, 1H), 7.62 (s, 1H), 7.48 (s, 1H), 6.38- 6.23 (m, 1H), 6.13-6.03 (m, 1H), 5.72-5.61 (m, 1H), 4.76-4.61 (m, 1H), 4.33 (s, 1H), 4.28 (s, 1H), 4.04 (s, 1H), 3.99 (s, 1H), 3.40 (m, 1H), 3.30 (m, 1H), 2.92-2.79 (m, 1H), 2.80-2.61 (m, 5H), 2.56 (s, 3H), 2.48 (s, 3H), 1.95-1.87 (m, 1H), 1.90 (s, 3H), 1.88-1.78 (m, 3H), 1.68-1.54 (m, 1H), 1.49- 1.28 (m, 1H); UPLC-MS-2a: Rt = 0.88 min; MS m/z [M + H]⁺ 548.3/ 550.3; C-SFC-39 (mobile phase: CO₂/[CH₃OH + 0.05% DEA] gradient from 95/5 to 60/40): Rt = 5.49 min, Example 38a: C-SFC- 39 (mobile phase: CO₂/[CH₃OH + 0.05% DEA] gradient from 95/5 to 60/40): Rt = 4.90 min. |
| | 5-(1-(2-acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 1-methyloctahydro-2H- | | |
| | pyrrolo[3,2-c]pyridin-2- | | |
| | one | | |
| 39a/39b | 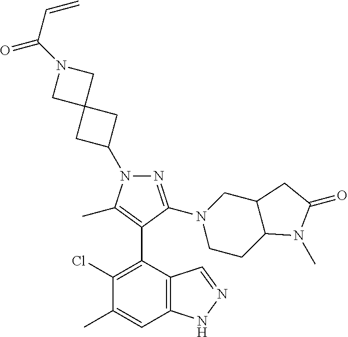 [SMILES: C=CC(=O)N1CC2(CC(n3nc(N4CCC5C(CC(=O)N5C)C4)c(-c4c(Cl)c(C)cc5[nH]ncc45)c3C)C2)C1] | Using Method-3 from Intermediate C26a (Step 3) and C-SFC-2 (mobile phase: CO₂/IPA 62/38); Example 39a = 1^{st} eluting isomer, Example 39b = 2^{nd} eluting isomer | Example 39b: ¹H NMR (600 MHz, DMSO-d₆) δ 13.06 (s, 1H), 7.63 (s, 1H), 7.49 (s, 1H), 6.38- 6.25 (m, 1H), 6.15-6.04 (m, 1H), 5.71-5.63 (m, 1H), 4.79-4.64 (m, 1H), 4.34 (s, 1H), 4.28 (s, 1H), 4.05 (s, 1H), 3.99 (s, 1H), 3.61- 3.52 (m, 1H), 3.15-3.04 (m, 1H), 2.87-2.81 (m, 1H), 2.78-2.71 (m, 2H), 2.69-2.64 (m, 3H), 2.58- 2.53 (m, 1H), 2.53 (s, 3H), 2.48 (s, 3H), 2.10 (dd, 1H), 2.00 (t, 1H), 1.95 (s, 3H), 1.86-1.74 (m, 1H), 1.70-1.64 (m, 1H), 0.95- 0.89 (m, 1H); UPLC-MS-2a: Rt = 0.90 min; MS m/z [M + H]⁺ 548.3/ 550.3; C-SFC-3 (mobile phase: CO₂/IPA 60/40): Rt = 1.84 min), Example 39a: C-SFC-3 (mobile phase: CO₂/IPA 60/40): Rt = 1.10 min. |
| | 5-(1-(2-acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 1-methyloctahydro-2H- | | |
| | pyrrolo[3,2-c]pyridin-2- | | |
| | one | | |
| 40a/40b | 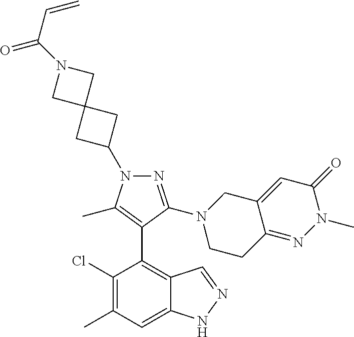 [SMILES: C=CC(=O)N1CC2(CC(n3nc(N4CCc5nn(C)c(=O)cc5C4)c(-c4c(Cl)c(C)cc5[nH]ncc45)c3C)C2)C1] | Using Method- 3bg from 2- methyl-5,6,7,8- tetrahydropyrido [4,3-c]pyridazin- 3(2H)-one (Step 1) and C-SFC-2 (mobile phase: CO₂/MeOH 68/32); Example 40a = 1^{st} eluting isomer, Example 40b = 2^{nd} eluting isomer | Example 40b: ¹H NMR (400 MHz, DMSO-d₆) δ 13.1 (s, 1H), 7.62 (s, 1H), 7.53 (s, 1H), 6.64 (m, 1H), 6.33 (m, 1H), 6.11 (m, 1H), 5.68 (m, 1H), 4.72 (m, 1H), 4.35 (s, 1H), 4.27 (m, 1H), 4.22- 4.11 (m, 2H), 4.05 (s, 1H), 3.99 (m, 1H), 3.55 (s, 3H), 2.98-2.92 (m, 2H), 2.78-2.65 (m, 4H), 2.49 (s, 3H), 2.43-2.37 (m, 1H), 2.23- 2.16 (m, 1H), 1.96 (s, 3H). UPLC- MS-2d: Rt = 3.89 min; MS m/z [M + H]⁺ 559.2/561.2; C-SFC-3 (mobile phase: CO₂/MeOH 70/30): Rt = 2.78 min, Example 40a: C-SFC-3 (mobile phase: CO₂/MeOH 70/30): Rt = 1.71 min. |
| | 6-(1-(2-acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 2-methyl-5,6,7,8- | | |
| | tetrahydropyrido[4,3- | | |
| | c]pyridazin-3(2H)-one | | |
| 41a/41b | 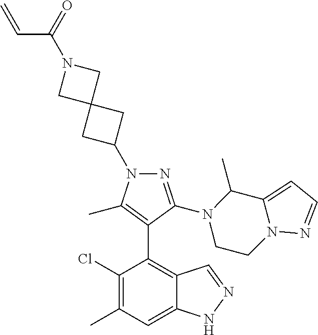 [SMILES: C=CC(=O)N1CC2(CC(n3nc(N4CCn5nccc5C4C)c(-c4c(Cl)c(C)cc5[nH]ncc45)c3C)C2)C1] | Using Method- 3abh from Intermediate A62a (Step 1) and C-SFC-24 (mobile phase: CO₂/IPA: 50/50); Example 41a = 1^{st} eluting isomer, Example 41b = 2^{nd} eluting isomer | Example 41b: ¹H NMR (400 MHz, DMSO-d₆) δ 13.1 (s, 1H), 7.63 (s, 1H), 7.51 (s, 1H), 7.27 (m, 1H), 6.31 (m, 1H), 6.10 (m, 1H), 5.90 (m, 1H), 5.68 (m, 1 H), 4.74 (m, 1H), 4.48 (m, 1H), 4.34 (m, 1H), 4.26 (m, 1H), 4.04 (m, 1H), 4.02-3.95 (m, 2H), 3.82- 3.75 (m, 1H), 3.54-3.45 (m, 1H), 3.25 (m, 1H), 2.75-2.67 (m, 4H), 2.50 (s, 3H), 1.97 (s, 3H), 1.22 (m, 3H); UPLC-MS-2b: Rt = 0.97 min; MS m/z [M + H]⁺ 531.2/ 533.2; C-SFC-6 (mobile phase: CO₂//IPA:50/50): Rt = 1.66 min, Example 41a: C-SFC-6 (mobile phase: CO₂/IPA:50/50): Rt = 1.09 min. |
| | 1-(6-(4-(5-chloro-6- | | |
| | methyl-1H-indazol-4-yl)- | | |
| | 5-methyl-3-(4-methyl-6,7- | | |
| | dihydropyrazolo[1,5- | | |
| | a]pyrazin-5(4H)-yl)-1H- | | |
| | pyrazol-1-yl)-2- | | |
| | azaspiro[3.3]heptan-2- | | |
| | yl)prop-2-en-1-one | | |
| 42a/42b | 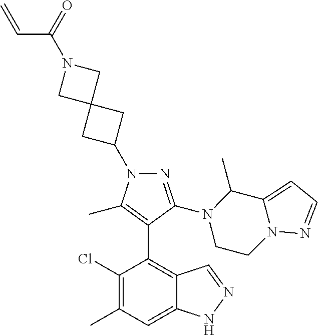 [SMILES: C=CC(=O)N1CC2(CC(n3nc(N4CCn5nccc5C4C)c(-c4c(Cl)c(C)cc5[nH]ncc45)c3C)C2)C1] | Using Method- 3abh from Intermediate A62b (Step 1) and C-SFC-5 (mobile phase: CO₂/IPA: 57/43); Example 42a = 1^{st} eluting isomer, Example 42b = 2^{nd} eluting isomer | Example 42b: ¹H NMR (400 MHz, DMSO-d₆) δ 13.1 (s, 1H), 7.66 (s, 1H), 7.52 (s, 1H), 7.28 (m, 1H), 6.31 (m, 1H), 6.11 (m, 1H), 5.92 (m, 1H), 5.67 (m, 1H), 4.73 (m, 1H), 4.45 (m, 1H), 4.34 (s, 1H), 4.27 (s, 1H), 4.04 (s, 1H), 4 (s, 1H), 3.99-3.94 (m, 1H), 3.84 (m, 1H), 3.62-3.52 (m, 1H), 3.40- 3.34 (m, 1H), 2.79-2.66 (m, 4H), 2.49 (s, 3H), 1.94 (s, 3H), 1.01 (m, 3H); UPLC-MS-2b: Rt = 0.97 min; MS m/z [M + H]⁺ 531.2/ 533.2; C-SFC-6 (mobile phase: CO₂//IPA:57/43): Rt = 2.82 min, Example 42a: C-SFC-6 (mobile phase: CO₂/IPA:57/43): Rt = 1.67 min. |
| | 1-(6-(4-(5-chloro-6- | | |
| | methyl-1H-indazol-4-yl)- | | |
| | 5-methyl-3-(4-methyl-6,7- | | |
| | dihydropyrazolo[1,5- | | |
| | a]pyrazin-5(4H)-yl)-1H- | | |
| | pyrazol-1-yl)-2- | | |
| | azaspiro[3.3]heptan-2- | | |
| | yl)prop-2-en-1-one | | |
| 43a/43b | 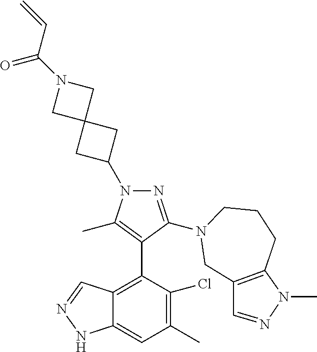 [SMILES: C=CC(=O)N1CC2(CC(n3nc(N4CCCc5c(cnn5C)C4)c(-c4c(Cl)c(C)cc5[nH]ncc45)c3C)C2)C1] | Using Method-3a from (CAS [1505428-23-8]) (Step 1) and C- SFC-2 (mobile phase: CO₂/IPA 65/35); Example 43a = 1^{st} eluting isomer, Example 43b = 2^{nd} eluting isomer | Example 43b: ¹H NMR (600 MHz, DMSO-d₆) δ 13.1 (s, 1H), 7.52 (s, 1H), 7.50 (s, 1H), 6.44 (m, 1H), 6.31 (m, 1H), 6.10 (m, 1H), 5.68 (m, 1H), 4.62 (m, 1H), 4.32 (s, 1H), 4.28 (s, 1H), 4.03 (s, 1H), 4.00 (s, 1H), 3.87 (m, 2H), 3.60 (s, 3H), 3.34 (m, 2H), 2.75- 2.59 (m, 6H), 2.50 (s, 3H), 1.84 (s, 3H), 1.57 (m, 2H); UPLC-MS- 1e: Rt = 0.93 min; MS m/z [M + H]⁺ 545.2/547.2; C-SFC-3 (mobile phase: CO₂/IPA:65/35): Rt = 2.95 min, Example 43a: C-SFC- 3 (mobile phase: CO₂/IPA: 65/35): Rt = 2.00 min. |
| | 1-(6-(4-(5-chloro-6- | | |
| | methyl-1H-indazol-4-yl)- | | |
| | 5-methyl-3-(1-methyl- | | |
| | 4,6,7,8- | | |
| | tetrahydropyrazolo[4,3- | | |
| | c]azepin-5(1H)-yl)-1H- | | |
| | pyrazol-1-yl)-2- | | |
| | azaspiro[3.3]heptan-2- | | |
| | yl)prop-2-en-1-one | | |
| 44a/44b | 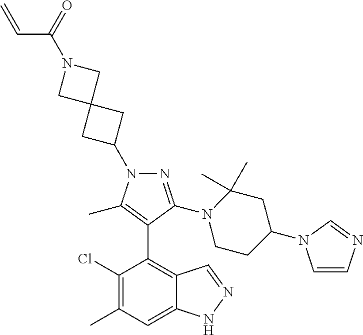 [SMILES: C=CC(=O)N1CC2(CC(n3nc(N4CCC(n5ccnc5)CC4(C)C)c(-c4c(Cl)c(C)cc5[nH]ncc45)c3C)C2)C1] | Using Method-3 from Intermediate C109a (Step 3) and RP-HPLC-11 (mobile phase: A: [5 mM ammonium bicarbonate + 0.1% NH₃ in water/B: CH₃CN, gradient: 25 to 40% B in 58 min, 40% B for 21 min, 40 to 100% B in 2 min then 100 to 25% in 6 min); Example 44a = 1^{st} eluting isomer, Example 44b = 2^{nd} eluting isomer | Example 44b: ¹H NMR (400 MHz, DMSO-d₆) δ 13.01 (s, 1H), 7.59 (s, 2H), 7.46 (s, 1H), 7.05 (s, 1H), 6.84 (s, 1H), 6.35 (m, 1H), 6.12 (d, 1H), 5.69 (d, 1H), 4.81 (m, 1H), 4.35-4.30 (m, 3H), 4.07 (s, 1H), 4.02 (s, 1H), 3.34 (m, 1H), 2.97 (m, 1H), 2.73-2.68 (m, 4H), 2.01 (s, 3H), 1.88 (m, 1H), 1.73 (d, 1H), 1.47-1.36 (m, 2H), 1.23 (s, 3H), 0.82 (s, 1.5H), 0.81 (s, 1.5H); UPLC-MS-14 ((mobile phase: A: [5 mM Ammonium bicarbonate + 0.1 % NH₃ in water]/B: [CH₃CN + 0.1% NH₃]; gradient: 90:10 at 0.01 min, 50:50 at 10.0 min, 30:70 at 15.0 min, 10:90 at 20.0 min): Rt = 14.13 min, MS m/z, [M + H]⁺ 573.3/575.4; RP-HPLC-8: Rt = 5.81 min. Example 44a: RP- HPLC-8: Rt = 5.68 min. |
| | 1-(6-(3-(4-(1H-imidazol- | | |
| | 1-yl)-2,2- | | |
| | dimethylpiperidin-1-yl)-4- | | |
| | (5-chloro-6-methyl-1H- | | |
| | indazol-4-yl)-5-methyl- | | |
| | 1H-pyrazol-1-yl)-2- | | |
| | azaspiro[3.3]heptan-2- | | |
| | yl)prop-2-en-1-one | | |
| 45a/45b | 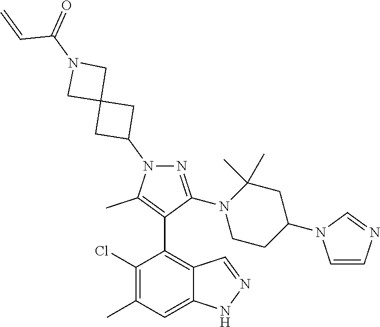 [SMILES: C=CC(=O)N1CC2(CC(n3nc(N4CCC(n5ccnc5)CC4(C)C)c(-c4c(Cl)c(C)cc5[nH]ncc45)c3C)C2)C1] | Using Method-3 from Intermediate C109b (Step 3) and RP-HPLC-11 (mobile phase: A: [5 mM ammonium bicarbonate + 0.1% NH₃ in water/B: CH₃CN, gradient: 25 to 40% B in 58 min, 40% B for 21 min, 40 to 100% B in 2 min then 100 to 25% in 6 min); Example 45a = 1^{st} eluting isomer, Example 45b = 2^{nd} eluting isomer | Example 45a: ¹H NMR (400 MHz, DMSO-d₆) δ 13.04 (s, 1H), 7.66 (s, 1H), 7.57 (s, 1H), 7.48 (s, 1H), 7.04 (s, 1H), 6.85 (s, 1H), 6.34 (m, 1H), 6.14 (d, 1H), 5.71 (d, 1H), 4.78 (m, 1H), 4.36-4.31 (m, 3H), 4.07 (s, 1H), 4.02 (s, 1H), 3.35 (m, 1H), 3.09 (m, 1H), 2.76-2.68 (m, 5H), 2.47 (s, 3H), 2.00 (s, 3H), 1.80 (d, 1H), 1.72 (m, 1H), 1.56 (m, 1H), 1.37 (s, 3H), 1.02 (s, 1.5H), 1.01 (s, 1.5H); UPLC-MS-14 ((mobile phase: A: [5 mM Ammonium bicarbonate + 0.1 % NH₃ in water]/B: [CH₃CN + 0.1% NH₃]; gradient: 90:10 at 0.01 min, 50:50 at 10.0 min, 30:70 at 15.0 min, 10:90 at 20.0 min): Rt = 13.6 min, MS m/z, [M + H]⁺ 573.3/ 575.4; RP-HPLC-8: Rt = 5.67 min. Example 45b: RP-HPLC-8: Rt = 5.82 min |
| | 1-(6-(3-(4-(1H-imidazol- | | |
| | 1-yl)-2,2- | | |
| | dimethylpiperidin-1-yl)-4- | | |
| | (5-chloro-6-methyl-1H- | | |
| | indazol-4-yl)-5-methyl- | | |
| | 1H-pyrazol-1-yl)-2- | | |
| | azaspiro[3.3]heptan-2- | | |
| | yl)prop-2-en-1-one | | |
| 46 | 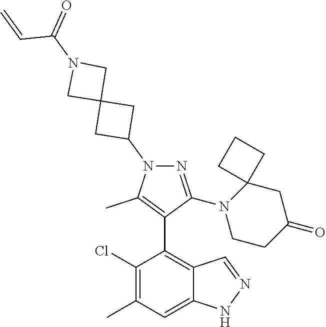 [SMILES: C=CC(=O)N1CC2(CC(n3nc(N4CCC(=O)CC45CCC5)c(-c4c(Cl)c(C)cc5[nH]ncc45)c3C)C2)C1] | Using Method-3 from Intermediate C110a (Step 4). | Example 46: 1H NMR (400 MHz, Methanol-d₄) δ 7.74 (s, 1H), 7.49 (s, 1H), 6.41 (m, 1H), 6.28 (m, 1H), 5.78 (m, 1H), 4.81 (m, 1H), 4.45 (s, 1H), 4.39 (s, 1H), 4.22 (s, 1H), 4.15 (s, 1H), 3.23 (d, 1H), 2.96-2.80 (m, 6H), 2.57 (s, 3H), 2.36 (m, 1H), 2.17-2.12 (m, 2H), 2.05 (s, 3H), 2.00 (m, 1H), 1.90- 1.65 (m, 4H); UPLC-MS-5: Rt = 1.69 min, MS m/z [M + H]⁺ 533.5/ 535.5. |
| | 5-(1-(2-acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 5-azaspiro[3.5]nonan-8- | | |
| | one | | |
| 47 | 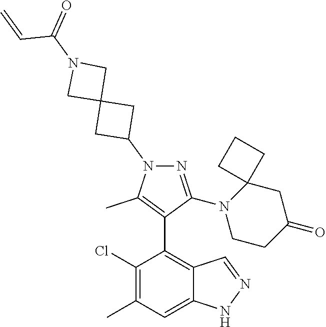 [SMILES: C=CC(=O)N1CC2(CC(n3nc(N4CCC(=O)CC45CCC5)c(-c4c(Cl)c(C)cc5[nH]ncc45)c3C)C2)C1] | Using Method-3 from Intermediate C110b (Step 4) | Example 47: 1H NMR (400 MHz, Methanol-d₄) δ 7.74 (s, 1H), 7.49 (s, 1H), 6.39 (m, 1H), 6.29 (d, 1H), 5.78 (d, 1H), 4.81 (m, 1H), 4.46 (s, 1H), 4.39 (s, 1H), 4.22 (s, 1H), 4.16 (s, 1H), 3.23 (d, 1H), 2.97-2.81 (m, 6H), 2.57 (s, 3H), 2.34 (m,1H), 2.17 (m, 2H), 2.05 (s, 3H), 2.00 (m, 1H), 1.87-1.72 (m, 4H); UPLC-MS-5: Rt = 1.72 min, MS m/z [M + H]⁺ 533.5/ 535.5. |
| | 5-(1-(2-acryloyl-2- | | |
| | azaspiro[3.3]heptan-6- | | |
| | yl)-4-(5-chloro-6-methyl- | | |
| | 1H-indazol-4-yl)-5- | | |
| | methyl-1H-pyrazol-3-yl)- | | |
| | 5-azaspiro[3.5]nonan-8- | | |
| | one | | |
| 48a/48b | 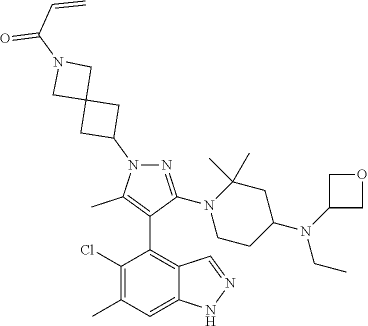 [SMILES: C=CC(=O)N1CC2(CC(n3nc(N4CCC(N(CC)C5COC5)CC4(C)C)c(-c4c(Cl)c(C)cc5[nH]ncc45)c3C)C2)C1] | Using Method-3 from Intermediate C112 (Step 4) and RP-HPLC-5 (mobile phase: A: [5 mM ammonium bicarbonate + 0.1% NH₃ in water]/B: CH₃CN, gradient: 25 to 40% B in 25 min, 40% B for 14 min, 40 to 100% in 2 min, 100 to 25% in 5 min). Example 48a = 1^{st} eluting isomer, Example 48b = 2^{nd} eluting isomer. | Example 48b: ¹H NMR (400 MHz, Methanol-d₄) δ 7.62 (s, 1H), 7.46 (s, 1H), 6.39-6.35 (m, 1H), 6.29 (m, 1H), 5.78-5.75 (m, 1H), 4.81 (m, 1H), 4.62-4.51 (m, 4H), 4.45 (s, 1H), 4.42 (s, 1H), 4.22 (s, 1H), 4.19 (s, 1H), 4.06- 4.29 (m, 1H), 3.04 (m, 1H), 2.93- 2.81 (m, 6H), 2.61 (m, 2H), 2.57 (s, 3H), 2.09 (s, 3H), 1.56 (m, 1H), 1.35 (m, 1H), 1.21 (s, 3H), 1.71 (m, 1H), 1.01-0.94 (m, 4H), 0.76 (s, 1.5H), 0.75 (s, 1.5H). RP-HPLC-8: Rt = 6.10 min; UPLC-MS-7: Rt = 3.90 min, MS m/z [M + H]⁺ 606.4/ 608.4. Example 48a: RP-HPLC-8: Rt = 6.02 min. |
| | 1-(6-(4-(5-chloro-6- | | |
| | methyl-1H-indazol-4-yl)- | | |
| | 3-(4-(ethyl(oxetan-3- | | |
| | yl)amino)-2,2- | | |
| | dimethylpiperidin-1-yl)-5- | | |
| | methyl-1H-pyrazol-1-yl)- | | |
| | 2-azaspiro[3.3]heptan-2- | | |
| | yl)prop-2-en-1-one | | |
| 49a/49b | 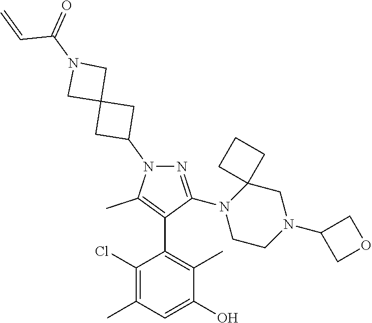 [SMILES: C=CC(=O)N1CC2(CC(n3nc(N4CCN(C5COC5)CC45CCC5)c(-c4c(C)c(O)cc(C)c4Cl)c3C)C2)C1] | Using Method-3 from Intermediate C73 (Step 4) and C-HPLC-34 (mobile phase: hexane/ [IPA/MeOH 50/50], gradient: 20 to 70% in 15 min, 20% for 20 min). Example 49a = 1st eluting isomer, Example 49b = 2nd eluting isomer | Example 49a: 1H NMR (400 MHz, Methanol-d₄) δ 6.69 (s, 1H), 6.40 (m, 1H), 6.28 (m, 1H), 5.78 (m, 1H), 4.78-4.60 (m, 5H), 4.43 (s, 1H), 4.38 (s, 1H), 4.20 (s, 1H), 4.14 (s, 1H), 3.48 (m, 2H), 2.95-2.75 (m, 6H), 2.67-2.49 (m, 2H), 2.34 (m, 1H), 2.30 (s, 3H), 2.28 (m, 1H), 2.00-2.18 (m, 3H), 1.94 (m, 6H), 1.74 (m, 2H); UPLC-MS-7: Rt = 3.93 min, MS m/z [M + H]⁺ 566.2/569.2; C- HPLC-29 (mobile phase: hexane/[IPA/MeOH 50/50] 65:35), Rt = 5.85 min. Example 49b: C-HPLC-29: (mobile phase: hexane/[IPA/MeOH 50/50] 65:35), Rt = 8.26 min. |
| | 1-(6-(4-(2-chloro-5- | | |
| | hydroxy-3,6- | | |
| | dimethylphenyl)-5- | | |
| | methyl-3-(8-(oxetan-3- | | |
| | yl)-5,8- | | |
| | diazaspiro[3.5]nonan-5- | | |
| | yl)-1H-pyrazol-1-yl)-2- | | |
| | azaspiro[3.3]heptan-2- | | |
| | yl)prop-2-en-1-one | | |
| 50a/50b | 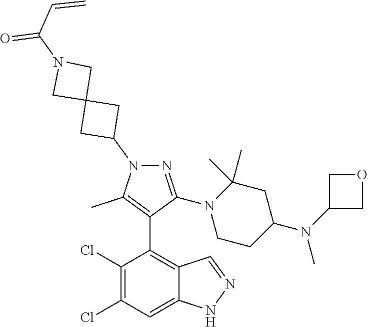 [SMILES: C=CC(=O)N1CC2(CC(n3nc(N4CCC(N(C)C5COC5)CC4(C)C)c(-c4c(Cl)c(Cl)cc5[nH]ncc45)c3C)C2)C1] | Using Method-3c from Intermediate C102a (Step 2) and D6 (Step 3) and RP-HPLC-11 (mobile phase: A: [10 mM ammonium acetate in water]/ B: CH₃CN, gradient: 35 to 40% B in 25 min, 40% B for 3 min, 40 to 100% B in 2 min then 100 to 35% B in 5 min). Example 50a = 1^{st} eluting isomer, Example 50b = 2^{nd} eluting isomer | Example 50b: ¹H NMR (400 MHz, Methanol-d₄) δ 7.75 (s, 1H), 6.68 (s, 1H), 6.37-6.35 (m, 1H), 6.29 (d, 1H), 5.78-5.75 (d, 1H), 4.82 (m, 1H), 4.60-4.58 (m, 4H), 4.45 (s, 1H), 4.41 (s, 1H), 4.22 (s, 1H), 4.19 (s, 1H), 3.93 (m, 1H), 3.04 (m, 1H), 2.91-2.82 (m, 5H), 2.63 (m, 1H), 2.10 (s, 3H), 2.08 (s, 3H), 1.59 (m, 1H), 1.38 (m, 1H), 1.24 (m, 1H), 1.22 (m, 1H), 1.06 (s, 3H), 1.03 (m, 1H), 0.76 (s, 3H); UPLC-MS-18 (mobile phase: A: [5 mM ammonium acetate + 0.1% NH₃ in water]/[CH₃CN + 0.1% NH₃] gradient 90:10 at 0.01 min, 50:50 at 10.0 min, 30:70 at 20.0 min, 0:100 at 25 min), Rt = 14.77 min, MS m/z [M + H]⁺ 612.2/614.2; RP-HPLC-8: Rt = 5.85 min. Example 50a: RP-HPLC-8: Rt = 5.75 min. |
| | 1-(6-(4-(5,6-dichloro-1H- | | |
| | indazol-4-yl)-3-(2,2- | | |
| | dimethyl-4- | | |
| | (methyl(oxetan-3- | | |
| | yl)amino)piperidin-1-yl)- | | |
| | 5-methyl-1H-pyrazol-1- | | |
| | yl)-2- | | |
| | azaspiro[3.3]heptan-2- | | |
| | yl)prop-2-en-1-one | | |
| 51a/51b | 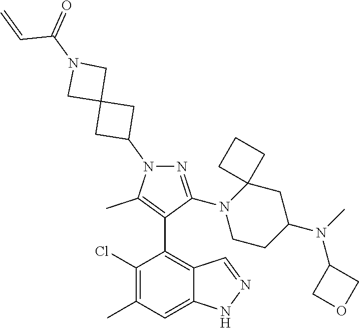 [SMILES: C=CC(=O)N1CC2(CC(n3nc(N4CCC(N(C)C5COC5)CC45CCC5)c(-c4c(Cl)c(C)cc5[nH]ncc45)c3C)C2)C1] | Using Method-3 from Intermediate C101b (Step 3) and RP-HPLC-5 (mobile phase: A: 0.1% NH₃ in water/B: CH₃CN, gradient: 35 to 40% B in 30 min, 40% B for 8 min, 40 to 100% B in 2 min, 100 to 35% in 5 min). Example 51a = 1^{st} eluting isomer, Example 51b = 2^{nd} eluting isomer | Example 51a: ¹H NMR (400 MHz, Methanol-d₄)) δ 7.71 (s, 1H), 7.45 (s, 1H), 6.42 (m, 1H), 6.29 (m, 1H), 5.78 (m, 1H), 4.80 (m, 1H), 4.65 (m, 4H), 4.46 (s, 1H), 4.42 (s, 1H), 4.23 (s, 1H), 4.19 (s, 1H), 4.03 (m, 1H), 3.01 (m, 1H), 2.87-2.84 (m, 5H), 2.61 (m, 1H), 2.56 (s, 3H), 2.28 (m, 1H), 2.19 (s, 1.5H), 2.18 (s, 1.5H), 2.07 (s, 3H), 1.95 (m, 3H), 1.80-1.72 (m, 4H), 1.32 (m, 2H); UPLC-MS-14 (mobile phase: A: [5 mM ammonium bicarbonate + 0.1% NH₃ in water]/B: [CH₃CN + 0.1% NH₃] gradient: 90:10 at 0.01 min, 50:50 at 10.0 min, 20:80 at 20.0 min, 0:100 at 25 min): Rt = 20.6 min, MS m/z [M + H]⁺ = 604.2/ 607.2, RP- HPLC-8: Rt = 5.87 min. Example 51b: RP-HPLC-8: Rt = 6.04 min. |
| | 1-(6-(4-(5-chloro-6- | | |
| | methyl-1H-indazol-4-yl)- | | |
| | 5-methyl-3-(8- | | |
| | (methyl(oxetan-3- | | |
| | yl)amino)-5- | | |
| | azaspiro[3.5]nonan-5-yl)- | | |
| | 1H-pyrazol-1-yl)-2- | | |
| | azaspiro[3.3]heptan-2- | | |
| | yl)prop-2-en-1-one | | |
| 52a/52b | 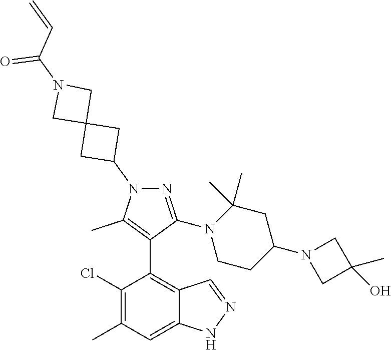 [SMILES: C=CC(=O)N1CC2(CC(n3nc(N4CCC(N5CC(C)(O)C5)CC4(C)C)c(-c4c(Cl)c(C)cc5[nH]ncc45)c3C)C2)C1] | Using Method-3c from Intermediate C104a (Step 2) and RP-HPLC-6 (mobile phase: A: 0.1% NH₃ in water/B: CH₃CN, gradient: 30 to 35% B in 35 min, 35% B for 15 min, 35 to 100% in 2 min, 100 to 30% in 4 min). Example 52a = 1st eluting isomer, Example 52b = 2nd eluting isomer | Example 52b: ¹H NMR (400 MHz, Methanol-d₄) δ 7.59 (s, 1H), 7.43 (s, 1H), 6.40-6.35 (m, 1H), 6.29 (d, 1H), 5.78-5.75 (d, 1H), 4.81 (m, 1H), 4.45 (s, 1H), 4.42 (s, 1H), 4.22 (s, 1H), 4.19 (s, 1H), 3.03-2.81 (m, 9H), 2.53 (s, 3H), 2.43 (m, 2H), 2.08 (s, 3H), 1.69 (m, 1H), 1.40 (m, 1H), 1.30 (s, 3H), 1.27 (m, 1H), 1.21 (s, 3H), 1.01 (m, 1H), 0.77 (s, 3H); RP-HPLC-8: Rt = 5.66 min. UPLC-MS-5: Rt = 1.47 min, MS m/z [M + H]⁺ 592.6/594.6. Example 52a: RP-HPLC-8: Rt = 5.60 min. |
| | 1-(6-(4-(5-chloro-6- | | |
| | methyl-1H-indazol-4-yl)- | | |
| | 3-(4-(3-hydroxy-3- | | |
| | methylazetidin-1-yl)-2,2- | | |
| | dimethylpiperidin-1-yl)-5- | | |
| | methyl-1H-pyrazol-1-yl)- | | |
| | 2-azaspiro[3.3]heptan-2- | | |
| | yl)prop-2-en-1-one | | |
| 53a/53b/ 53c/53d | 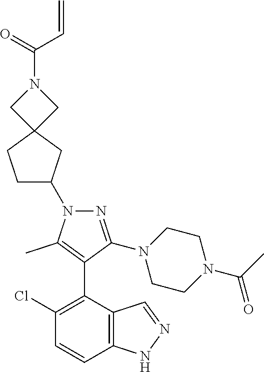 [SMILES: C=CC(=O)N1CC2(CCC(n3nc(N4CCN(C(C)=O)CC4)c(-c4c(Cl)ccc5[nH]ncc45)c3C)C2)C1] | Using Method-3 from Intermediate C13 (Step 2) and C-HPLC-8 (mobile phase: n- heptane/IPA 60/40); Example 53a = 1^{st} eluting isomer, Example 53b = 2^{nd} eluting isomer, Example 53c = 3^{rd} eluting isomer, Example 53d = 4^{th} eluting isomer | Example 53a: UPLC-MS-1d: Rt = 4.01 min; MS m/z [M + H]⁺ 522.5/ 524.5; C-HPLC-9 (mobile phase: n-heptane/IPA: 80/20): Rt = 7.15 min, Example 53d: UPLC-MS-1d: Rt = 4.18 min; MS m/z [M + H]⁺ 522.5/524.5; C- HPLC-9 (mobile phase: n- heptane/IPA: 80/20): Rt = 15.60 min, Example 53b: C-HPLC-9 (mobile phase: n-heptane/IPA: 80/20): Rt = 11.20 min. Example 53c: C-HPLC-9 (mobile phase: n-heptane/IPA: 80/20): Rt = 15.20 min. |
| | 1-(6-(3-(4- | | |
| | acetylpiperazin-1-yl)-4- | | |
| | (5-chloro-1H-indazol-4- | | |
| | yl)-5-methyl-1H-pyrazol- | | |
| | 1-yl)-2- | | |
| | azaspiro[3.4]octan-2- | | |
| | yl)prop-2-en-1-one | | |

### Method-4 for the Preparation of Example 54a and 54b: 1-(6-(4-(5-Chloro-6-methyl-1H-indazol-4-yl)-5-methyl-3-(8-(oxetan-3-yl)-5,8-diazaspiro[3.5]nonan-5-yl)-1H-pyrazol-1-yl)-2-azaspiro[3.3]heptan-2-yl)prop-2-en-1-one

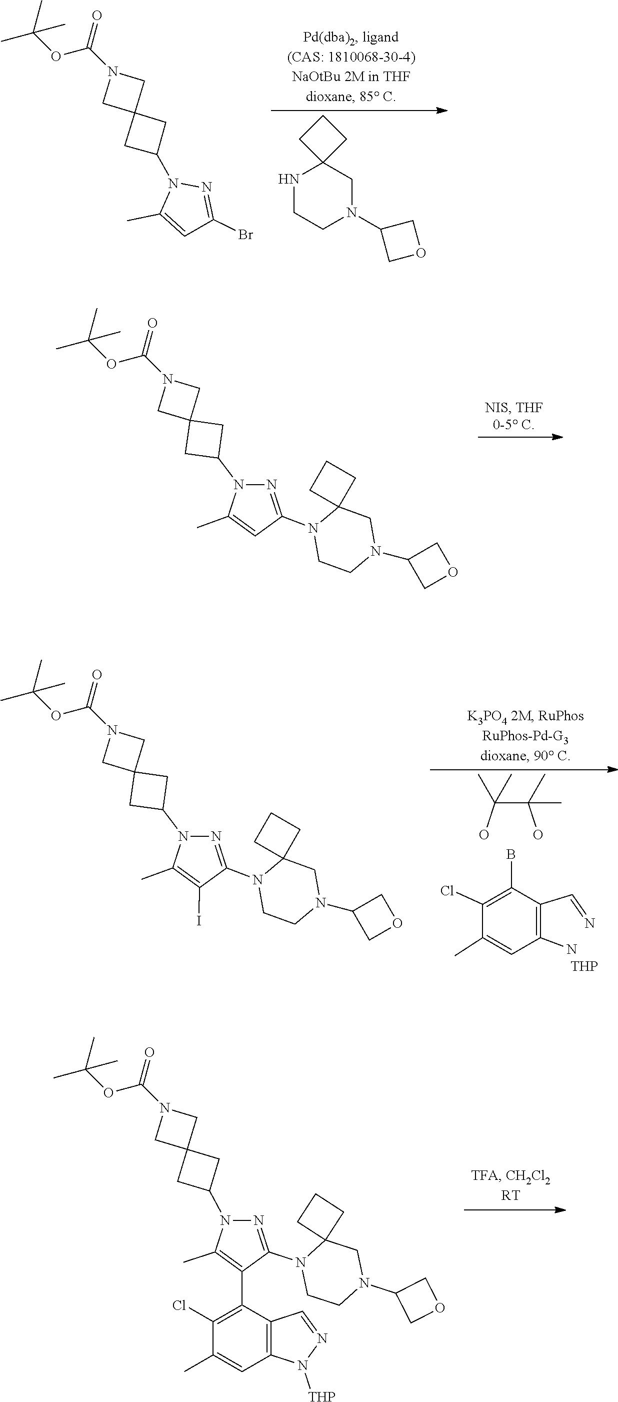

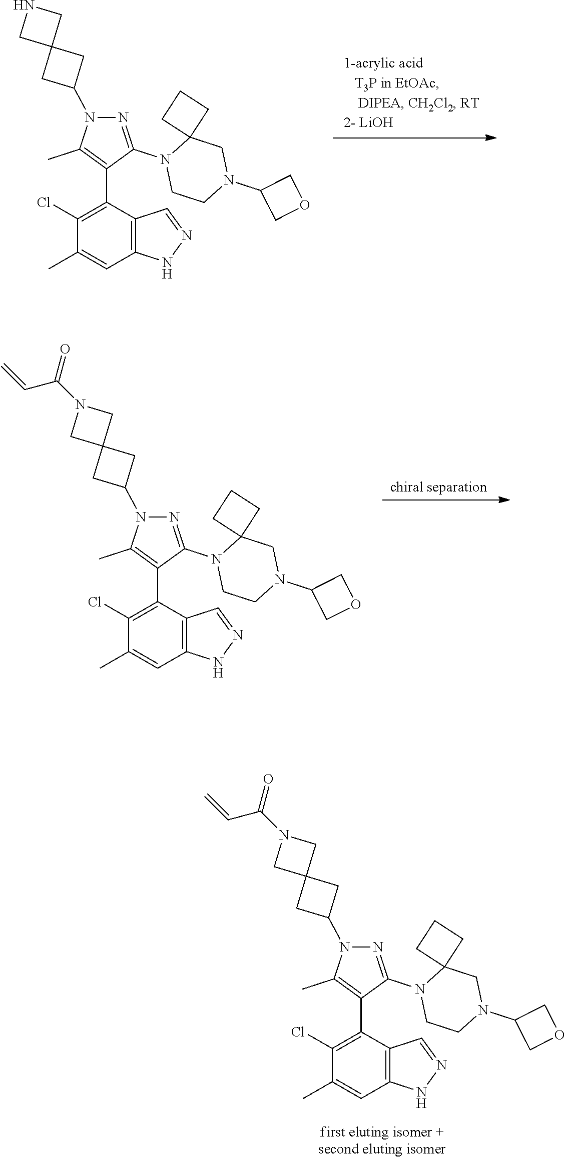 [SMILES: Cc1cc2[nH]ncc2c(-c2c(N3CCN(C4COC4)CC34CCC4)nn(C3CC4(CNC4)C3)c2C)c1Cl ; C=CC(=O)N1CC2(CC(n3nc(N4CCN(C5COC5)CC45CCC5)c(-c4c(Cl)c(C)cc5[nH]ncc45)c3C)C2)C1 ; C=CC(=O)N1CC2(CC(n3nc(N4CCN(C5COC5)CC45CCC5)c(-c4c(Cl)c(C)cc5[nH]ncc45)c3C)C2)C1]

### Step 1: Tert-butyl 6-(5-methyl-3-(8-(oxetan-3-yl)-5,8-diazaspiro[3.5]nonan-5-yl)-1H-pyrazol-1-yl)-2-azaspiro[3.3]heptane-2-carboxylate

In an ace tube, tert-butyl 6-(3-bromo-5-methyl-1H-pyrazol-1-yl)-2-azaspiro[3.3]heptane-2-carboxylate (Intermediate C1, 2.12 g, 5.95 mmol), 8-(oxetan-3-yl)-5,8-diazaspiro[3.5]nonane trifluoroacetate salt (Intermediate A1, 1.88 g, 6.55 mmol), Pd(dba)₂ (0.34 g, 0.59 mmol) and bis(3,5-bis(trifluoromethyl)phenyl)(2′,6′-bis(dimethylamino)-3,6-dimethoxybiphenyl-2-yl)phosphine (CAS: 1810068-30-4, 0.49 g, 0.65 mmol) were suspended in 1,4-dioxane (25 mL). NaOtBu (2M in THF, 7.44 mL, 14.9 mmol) was added, the vial was flushed with N₂ and the reaction mixture was placed in a preheated oil bath at 85° C. and stirred for 40 min. Then the RM was poured into an aq. sat. NaHCO₃ solution and extracted with CH₂Cl₂ (×3). The combined organic layers were dried (phase separator), concentrated under reduced pressure and the crude residue was purified by normal phase chromatography (eluent: MeOH in CH₂Cl₂ 0 to 5%) to give the title compound as a brown foam. UPLC-MS-2a: Rt=0.95 min; MS m/z [M+H]⁺ 458.4.

### Step 2: Tert-butyl 6-(4-iodo-5-methyl-3-(8-(oxetan-3-yl)-5,8-diazaspiro[3.5]nonan-5-yl)-1H-pyrazol-1-yl)-2-azaspiro[3.3]heptane-2-carboxylate

To an ice-cooled solution of tert-butyl 6-(5-methyl-3-(8-(oxetan-3-yl)-5,8-diazaspiro[3.5]nonan-5-yl)-1H-pyrazol-1-yl)-2-azaspiro[3.3]heptane-2-carboxylate (Step 1, 650 mg, 1.33 mmol) in THF (13 mL) was added NIS (315 mg, 1.40 mmol) and the mixture was stirred under N₂ atmosphere at 0° C. After completion (15 min), the reaction mixture was poured into 10% Na₂S₂O₃ aq. solution and extracted with CH₂Cl₂ (×2). The combined organic layers were washed with a sat. aq. NaHCO₃ solution, dried (phase separator) and concentrated to give the title product as a brown foam which was used without further purification in the next step. UPLC-MS-2a: Rt=1.11 min; MS m/z [M+H]⁺ 584.3.

### Step 3: Tert-butyl 6-(4-(5-chloro-6-methyl-1-(tetrahydro-2H-pyran-2-yl)-1H-indazol-4-yl)-5-methyl-3-(8-(oxetan-3-yl)-5,8-diazaspiro[3.5]nonan-5-yl)-1H-pyrazol-1-yl)-2-azaspiro[3.3]heptane-2-carboxylate

To a mixture of tert-butyl 6-(4-iodo-5-methyl-3-(8-(oxetan-3-yl)-5,8-diazaspiro[3.5]nonan-5-yl)-1H-pyrazol-1-yl)-2-azaspiro[3.3]heptane-2-carboxylate (Step 2, 835 mg, 1.33 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 652 mg, 1.73 mmol), RuPhos (62.1 mg, 0.13 mmol) and RuPhos-Pd-G3 (111 mg, 0.13 mmol) in dioxane (5.30 mL) was added K₃PO₄ (2 M in water, 2.00 mL, 4.00 mmol) and the reaction mixture was stirred at 90° C. for 1 h under a nitrogen atmosphere. The reaction mixture was poured into a sat. aq. NaHCO₃ solution and extracted with CH₂Cl₂ (×3). The combined organic extracts were dried (phase separator) and concentrated. The crude residue was diluted with THF (5 mL), SiliaMetS®Thiol (0.53 mmol) was added and the mixture swirled for 1 h at 40° C. The mixture was filtered, the filtrate was concentrated and the crude residue was purified by normal phase chromatography (eluent: EtOAc in c-hexane 0 to 100%) to give the title compound as a beige foam. UPLC-MS-2a: Rt=1.19 min; MS m/z [M+H]⁺ 706.5/708.4.

### Step 4: 5-(4-(5-chloro-6-methyl-1H-indazol-4-yl)-5-methyl-1-(2-azaspiro[3.3]heptan-6-yl)-1H-pyrazol-3-yl)-8-(oxetan-3-yl)-5,8-diazaspiro[3.5]nonane

To a solution of tert-butyl 6-(4-(5-chloro-6-methyl-1-(tetrahydro-2H-pyran-2-yl)-1H-indazol-4-yl)-5-methyl-3-(8-(oxetan-3-yl)-5,8-diazaspiro[3.5]nonan-5-yl)-1H-pyrazol-1-yl)-2-azaspiro[3.3]heptane-2-carboxylate (Step 3, 825 mg, 1.17 mmol) in CH₂Cl₂ (3.8 mL) was added TFA (2.70 mL, 35.0 mmol) and the solution was stirred at RT for 2.5 h. The RM was concentrated, co-evaporated with CH₂Cl₂ (×2) and dried under high vacuum overnight to give the title compound as a trifluoroacetate salt which was used in the next step without further purification. UPLC-MS-2a: Rt=0.65 min; MS m/z [M+H]⁺ 522.3/524.3.

### Step 5: 1-(6-(4-(5-chloro-6-methyl-1H-indazol-4-yl)-5-methyl-3-(8-(oxetan-3-yl)-5,8-diazaspiro[3.5]nonan-5-yl)-1H-pyrazol-1-yl)-2-azaspiro[3.3]heptan-2-yl)prop-2-en-1-one

A mixture of acrylic acid (0.12 mL, 1.75 mmol), propylphosphonic anhydride (50% in EtOAc, 1.17 mL, 1.26 mmol) and DIPEA (3.99 mL, 23.3 mmol) in CH₂Cl₂ (18 mL) was stirred for 20 min and then added under a nitrogen atmosphere to a solution of 5-(4-(5-chloro-6-methyl-1H-indazol-4-yl)-5-methyl-1-(2-azaspiro[3.3]heptan-6-yl)-1H-pyrazol-3-yl)-8-(oxetan-3-yl)-5,8-diazaspiro[3.5]nonane trifluoroacetate (Step 4, 1.16 mmol) in CH₂Cl₂ (9 mL). The reaction mixture was stirred at RT for 20 min. After completion of the reaction, LiOH (2M, 2.91 mL, 5.82 mmol) was added and the mixture was vigorously stirred at RT for 30 min to remove the side product resulting from reaction of the acryloyl chloride with the indazole NH. The reaction mixture was poured into a sat. aq. NaHCO₃ solution and extracted with CH₂Cl₂ (×2). The combined organic extracts were concentrated, dried (phase separator) and the crude residue was purified by normal phase chromatography (eluent: MeOH in CH₂Cl₂ 0 to 6%) to give the title compound as a light yellow foam. The isomers were separated by chiral SFC (C-SFC-1; mobile phase: CO₂/[IPA+0.1% Et₃N] 72/28) to give after lyophilization (CH₃CN/water) the title compound Example 54b as the second eluting peak (white solid): ¹H NMR (600 MHz, DMSO-d₆) δ 13.0 (s, 1H), 7.57 (s, 1H), 7.44 (s, 1H), 6.31 (m, 1H), 6.10 (m, 1H), 5.68 (m, 1H), 4.74 (m, 1H), 4.47 (d, 1H), 4.46 (d, 1H), 4.38-4.33 (m, 3H), 4.29 (m, 1H), 4.06 (s, 1H), 4.00 (m, 1H), 3.27 (m, 1H), 2.80-2.66 (m, 6H), 2.47 (s, 3H), 2.30-2.21 (m, 2H), 2.18-2.08 (m, 2H), 1.99 (s, 3H), 1.98 (m, 1H), 1.90 (m, 1H), 1.83 (m, 1H), 1.65-1.56 (m, 3H). UPLC-MS-2e: Rt=3.49 min; MS m/z [M+H]⁺ 576.4/578.4. C-SFC-3 (mobile phase: CO₂/[IPA+0.025% NH₃]: 72/28): Rt=2.60 min. The other isomer Example 54a was obtained as the first eluting peak: C-SFC-3 (mobile phase: CO₂/[IPA+0.025% NH₃]: 72/28): Rt=1.76 min.

Method-4a: similar to Method-4 except that in Step 2, NBS was used instead of NIS to prepare the corresponding 4-bromo-pyrazole.

Method-4b: similar to Method-4 except that in Step 2, NBS in acetonitrile was used instead of NIS in THF to prepare the corresponding 4-bromo-pyrazole.

Method-4c: similar to Method-4 except that in Step 1, 2-[bis(3,5-trifluoromethylphenylphosphino)-3,6-dimethoxy]-2′,6′-di-i-propoxy-1,1′-biphenyl (CAS: 1810068-31-5) was used as a ligand instead of bis(3,5-bis(trifluoromethyl)phenyl)(2′,6′-bis(dimethylamino)-3,6-dimethoxybiphenyl-2-yl)phosphine (CAS: 1810068-30-4).

Method-4d: similar to Method-4 except Step 5 was performed using HBTU in acetonitrile instead of propylphosphonic anhydride in CH₂Cl₂.

Method-5e: similar to Method-4 except Step 5 was performed using acryloyl chloride and NaHCO₃ as described in Method-8 Step 3.

Method-5f: similar to Method-4 except that in Step 2, acetonitrile was used instead of THF.

Method-5q: similar to Method-4 except that in Step 2 NIS, AIBN (0.1 eq) in acetonitrile was used instead of NIS in THF.

Method-4h: similar to Method-4 except that in Step 3 toluene was used as a solvent instead of dioxane.

Method-4i: similar to Method-4 except that in Step 1 toluene was used as a solvent instead of dioxane.

Method-4j: similar to Method-4 except that in Step 3 EtOH water was used as a solvent instead of dioxane.

Method-4k: similar to Method-4 except that in Step 2, DMF was used instead of THF.

The following examples 55a to 120b were prepared using analogous methods to Method-4 from intermediates (in Step 1, 2 or 3) described in the intermediates synthesis section or commercially available. When the final separation of isomers is performed by preparative HPLC using acidic conditions the purified fractions were extracted with a sat. aq. NaHCO₃ solution before lyophilization from a mixture of CH₃CN/H₂O to give the title compound as a free base.

| | | Method, | |
|---|---|---|---|
| | | intermediates | |
| | | (Step 1, 2 or 3) | |
| | | and chiral | |
| | | separation | |
| | | conditions used | |
| | | and order of | |
| Example | Structure | elution | Characterizing data |
| 55a/55b | 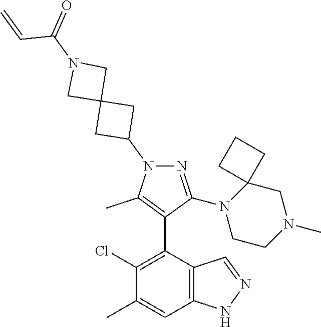 [SMILES: C=CC(=O)N1CC2(CC(n3nc(N4CCN(C)CC45CCC5)c(-c4c(Cl)c(C)cc5[nH]ncc45)c3C)C2)C1] | Using Method-4a from 8-methyl- 5,8- diazaspiro[3.5] nonane and C-SFC- 2 (mobile phase: CO₂/[IPA + 0.1% Et₃N] 75/25); Example 55a = 1^{st} eluting isomer, Example 55b = 2^{nd} eluting isomer | Example 55b: ¹H NMR (600 MHz, DMSO-d₆) δ 13.0 (s, 1H), 7.57 (s, 1H), 7.46 (s, 1H), 6.31 (m, 1H), 6.10 (m, 1H), 5.68 (m, 1H), 4.74 (m, 1H), 4.34 (s, 1H), 4.28 (s, 1H), 4.06 (s, 1H), 4.00 (s, 1H), 2.80-2.64 (m, 6H), 2.47 (s, 3H), 2.34 (m, 1H), 2.22 (m, 2H), 2.06-1.94 (m, 3H), 2.05 (s, 3H), 1.98 (s, 3H), 1.79 (m, 1H), 1.59 (m, 3H). UPLC-MS-2e: Rt = 3.28 min; MS m/z [M + H]⁺ 534.3/526.3; C-SFC-3 (mobile phase: CO₂/[IPA + 0.1% Et₃N] 75/25): Rt = 3.35 min, Example 55a: C-SFC-3 (mobile phase: CO₂/[IPA + 0.1% Et₃N] 75/25): Rt = 2.47 min. |
| | 1-(6-(4-(5-chloro-6-methyl- | | |
| | 1H-indazol-4-yl)-5-methyl- | | |
| | 3-(8-methyl-5,8- | | |
| | diazaspiro[3.5]nonan-5-yl)- | | |
| | 1H-pyrazol-1-yl)-2- | | |
| | azaspiro[3.3]heptan-2- | | |
| | yl)prop-2-en-1-one | | |
| 56a/56b | 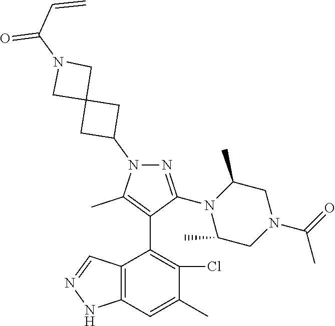 [SMILES: C=CC(=O)N1CC2(CC(n3nc(N4[C@@H](C)CN(C(C)=O)C[C@@H]4C)c(-c4c(Cl)c(C)cc5[nH]ncc45)c3C)C2)C1] | Using Method-4a from Intermediate A11 (Step 1) and C-SFC-2 (mobile phase: CO₂/IPA 68/32); Example 56a = 1^{st} eluting isomer, Example 56b = 2^{nd} eluting isomer | Example 56b: ¹H NMR (400 MHz, DMSO-d₆) δ 13.0 (s, 1H), 7.60 (s, 1H), 7.47 (s, 1H), 6.31 (m, 1H), 6.10 (m, 1H), 5.67 (m, 1H), 4.75 (m, 1H), 4.34 (s, 1H), 4.27 (s, 1H), 4.04 (s, 1H), 4.00 (s, 1H), 3.26 (m, 1H), 3.17-3.06 (m, 3H), 3.02 (m, 1H), 2.93 (m, 1H), 2.77-2.63 (m, 4H), 2.47 (s, 3H), 1.99 (s, 3H), 1.91 (s, 3H), 0.98 (m, 6H). UPLC-MS-2a: Rt = 0.94 min; MS m/z [M + H]⁺ 550.4/552.4; C-SFC-3 (mobile phase: CO₂/MeOH 70/30): Rt = 3.31 min, Example 56a: C- SFC-3 (mobile phase: CO₂/ MeOH 70/30): Rt = 1.98 min. |
| | 1-(6-(3-((2S,6S)-4-acetyl- | | |
| | 2,6-dimethylpiperazin-1- | | |
| | yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 57a/57b | 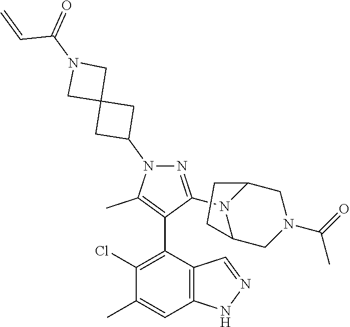 [SMILES: C=CC(=O)N1CC2(CC(n3nc(N4C5CCC4CN(C(C)=O)C5)c(-c4c(Cl)c(C)cc5[nH]ncc45)c3C)C2)C1] | Using Method- 4ae from Intermediate A12 (Step 1) and C- SFC-4 (mobile phase: CO₂/IPA 72/28); Example 57a = 1^{st} eluting isomer, Example 57b = 2^{nd} eluting isomer | Example 57b: ¹H NMR (400 MHz, DMSO-d₆) δ 13.10 (s, 1H), 7.67 (m, 1H), 7.50 (s, 1H), 6.31 (m, 1H), 6.11 (m, 1H), 5.68 (m, 1H), 4.70 (m, 1H), 4.34 (s, 1H), 4.30 (s, 1H), 4.05 (s, 1H), 4.01 (s, 1H), 3.79 (m, 1H), 3.66- 3.46 (m, 3H), 3.26-3.16 (m, 1H), 2.95 (m, 1H), 2.77-2.67 (m, 4H), 2.50 (s, 3H), 1.94 (s, 3H), 1.88 (s, 1.5H), 1.84 (1.5H), 1.66 (m, 2H), 1.50 (m, 1H), 1.33 (m, 1H). UPLC-MS-2e: Rt = 4.05 min; MS m/z [M + H]⁺ 548.2/550.2; C-SFC-3 (mobile phase: CO₂/IPA 70/30): Rt = 2.37 min, Example 57a: C- SFC-3 (mobile phase: CO₂/IPA 70/30): Rt = 1.92 min. |
| | 1-(6-(3-(3-acetyl-3,8- | | |
| | diazabicyclo[3.2.1]octan-8- | | |
| | yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 58a/58b | 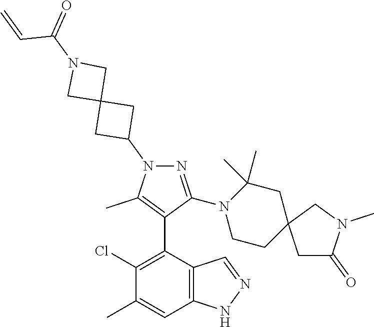 [SMILES: C=CC(=O)N1CC2(CC(n3nc(N4CCC5(CC(=O)N(C)C5)CC4(C)C)c(-c4c(Cl)c(C)cc5[nH]ncc45)c3C)C2)C1] | Using Method-4ci from C95a (Step 3) and C-HPLC- 23 (mobile phase: Hexane/IPA 78/22); Example 58a = 1^{st} eluting isomer, Example 58b = 2^{nd} eluting isomer | Example 58b: ¹H NMR (400 MHz, Methanol-d₄) δ 7.65 (s, 1H), 7.46 (s, 1H), 6.39-6.35 (m, 1H), 6.29-6.25 (d, 1H), 5.79- 5.76 (d, 1H), 4.82 (m, 1H), 4.46 (s, 1H), 4.43 (s, 1H), 4.23 (s, 1H), 4.19 (s, 1H), 3.30-3.16 (m, 3H), 3.10-2.90 (m, 2H), 2.93 (m, 1H), 2.82 (m, 2H), 2.79 (s, 3H), 2.56 (s, 3H), 2.35 (d, 1H), 2.21 (d, 1H), 2.07 (s, 3H), 1.45- 1.35 (m, 3H), 1.26 (m, 1H), 1.15 (s, 3H), 1.03 (s, 1.5H), 1.0 (s, 1.5H). UPLC-MS-5: Rt = 1.58 min, MS m/z [M + H]⁺ 591/593; C-HPLC-24 (mobile phase: Hexane/IPA 75/25), Rt = 15.7 min. Example 58a: C-HPLC-24 (mobile phase: Hexane/IPA 75/25), Rt = 9.63 min. |
| | 8-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-2,7,7- | | |
| | trimethyl-2,8- | | |
| | diazaspiro[4.5]decan-3- | | |
| | one | | |
| 59a/59b | 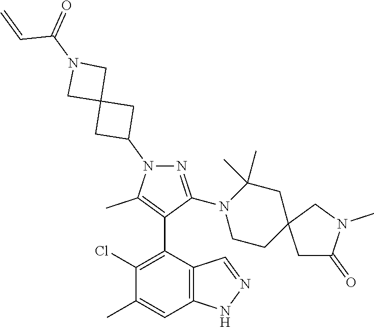 [SMILES: C=CC(=O)N1CC2(CC(n3nc(N4CCC5(CC(=O)N(C)C5)CC4(C)C)c(-c4c(Cl)c(C)cc5[nH]ncc45)c3C)C2)C1] | Using Method-4ci from C95b (Step 3) and C-HPLC- 23 (mobile phase: Hexane/IPA 78:22); Example 59a = 1^{st} eluting isomer, Example 59b = 2^{nd} eluting isomer | Example 59b: ¹H NMR (400 MHz, Methanol-d₄) δ 7.65 (s, 1H), 7.46 (s, 1H), 6.38-6.36 (m, 1H), 6.29-6.26 (d, 1H), 5.79- 5.76 (d, 1H), 4.82 (m, 1H), 4.46 (s, 1H), 4.42 (s, 1H), 4.23 (s, 1H), 4.19 (s, 1H), 3.34-3.21 (m, 3H), 3.18-3.05 (m, 2H), 2.92 (m, 1H), 2.91-2.87 (m, 2H), 2.80 (s, 3H), 2.56 (s, 3H), 2.34 (d, 1H), 2.19 (d, 1H), 2.07 (s, 3H), 1.45 (m, 1H), 1.42-1.31 (m, 3H), 1.15 (s, 1.5H), 1.14 (s, 1.5H), 1.05 (s, 1.5H), 1.03 (s, 1.5H). UPLC-MS-5: Rt = 1.57 min, MS m/z [M + H]⁺ 591/593; C-HPLC-24 (mobile phase: Hexane/IPA 75:25), Rt = 12.7 min. Example 59a: C-HPLC-24 (mobile phase: Hexane/IPA 75/25), Rt = 8.96 min. |
| | 8-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-2,7,7- | | |
| | trimethyl-2,8- | | |
| | diazaspiro[4.5]decan-3- | | |
| | one | | |
| 60 | 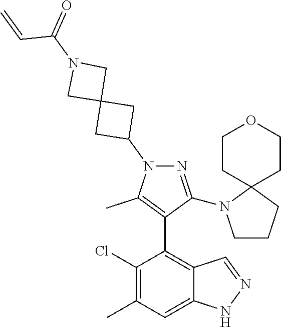 [SMILES: C=CC(=O)N1CC2(CC(n3nc(N4CCCC45CCOCC5)c(-c4c(Cl)c(C)cc5[nH]ncc45)c3C)C2)C1] | Using Method- 4bei from (CAS [51130-63-3]) (Step 1) | Example 60: ¹H NMR (400 MHz, MeOD) δ 7.60 (s, 1H), 7.46 (s, 1H), 6.45 (m, 1H), 6.29 (m, 1H), 5.78 (m, 1H), 4.79 (m, 1H), 4.56 (s, 1H), 4.45 (s, 1H), 4.20 (s, 1H), 4.18 (s, 1H), 3.98 (m, 2H), 3.55 (m, 2H), 3.05- 2.70 (m, 8H), 2.56 (s, 3H), 1.96 (s, 1H), 1.94 (m, 2H), 1.90 (m, 1H), 1.81 (m, 1H), 1.79-1.59 (m, 2H), 1.26-1.10 (m, 2H); UPLC-MS-5: Rt = 1.60 min, MS m/z [M + H]⁺ 535.6/537.6 RP- HPLC-8: Rt = 6.37 min. C-SFC- 49: Mobile phase (CO₂/ MeOH + .1% DEA gradient 5 to 50%), Rt = 4.70 min and 4.99 min. |
| | 1-(6-(4-(5-chloro-6-methyl- | | |
| | 1H-indazol-4-yl)-5-methyl- | | |
| | 3-(8-oxa-1- | | |
| | azaspiro[4.5]decan-1-yl)- | | |
| | 1H-pyrazol-1-yl)-2- | | |
| | azaspiro[3.3]heptan-2- | | |
| | yl)prop-2-en-1-one | | |
| 61a/61b | 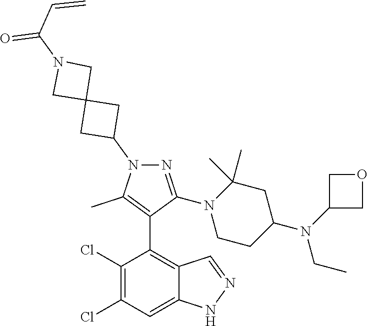 | Using Method- 4efh from Intermediate C107a (Step 2) and D6 (Step 3) and RP-HPLC-5 (mobile phase: A: [5 mM ammonium bicarbonate + 0.1% NH₃ in water]/B: CH₃CN, gradient 42% B for 32 min, 42 to 100% B in 2 min then 100 to 42% in 6 min); Example 61a = 1st eluting isomer, Example 61b = 2nd eluting isomer | Example 61b: ¹H NMR (400 MHz, Methanol-d₄) δ 7.76 (s, 1H), 7.68 (s, 1H), 6.41-6.40 (m, 1H), 6.28 (d, 1H), 5.78-5.75 (d, 1H), 4.80 (m, 1H), 4.52-4.62 (m, 4H), 4.44 (s, 1H), 4.41 (s, 1H), 4.22 (s, 1H), 4.19 (s, 1H), 4.01 (m, 1H), 3.01 (d, 1H), 2.98- 2.81 (m, 6H), 2.66 (m, 2H), 2.10 (s, 3H), 1.56 (m, 1H), 1.36 (m, 2H), 1.17 (s, 3H), 1.17 (m, 1H), 1.01-0.94 (t, 3H), 0.75 (s, 1.5H), 0.74 (s, 1.5H); UPLC-MS-14 (mobile phase: A: [5 mM ammonium bicarbonate + 0.1% NH₃ in water]/B: [CH₃CN + 0.1% NH₃] gradient: 90:10 at 0.01 min, 60:40 at 10.0 min, 30:70 at 20.0 min, 15:85 at 25.0 min): Rt = 18.6 min, MS m/z |
| | | | [M + H]⁺ 625.8/627.8: RP- |
| | 1-(6-(4-(5,6-dichloro-1H- | | HPLC-8: Rt = 6.25 min. |
| | indazol-4-yl)-3-(4- | | Example 61a: RP-HPLC-8: |
| | (ethyl(oxetan-3-yl)amino)- | | Rt = 6.20 min. |
| | 2,2-dimethylpiperidin-1-yl)- | | |
| | 5-methyl-1H-pyrazol-1-yl)- | | |
| | 2-azaspiro[3.3]heptan-2- | | |
| | yl)prop-2-en-1-one | | |
| 62a/62b | 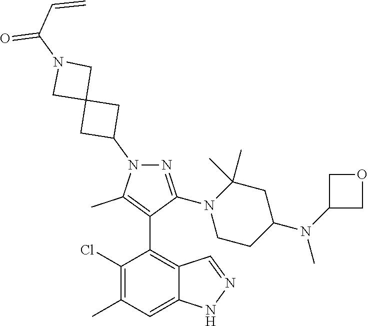 [SMILES: C=CC(=O)N1CC2(CC(n3nc(N4CCC(N(C)C5COC5)CC4(C)C)c(-c4c(Cl)c(C)cc5[nH]ncc45)c3C)C2)C1] | Using Method-4 from Intermediate C113 (Step 4) and C-HPLC-14 (mobile phase: heptane/ [heptane/TBME/ EtOH 50/40/10 + 0.1% Et₃N] 60/40); Example 62a = 1st eluting isomer, Example 62b = 2nd eluting isomer | Example 62b: UPLC-MS-2e: Rt = 3.44 min; MS m/z [M + H]⁺ 592.3/594.3; C-HPLC-16 (mobile phase: heptane/TBME/EtOH 50/40/10 + 0.1% Et3N): Rt = 22.4 min. Example 62a: C-HPLC-16 (mobile phase: heptane/TBME/EtOH 50/40/10 + 0.1% Et₃N): Rt = 19.5 min. |
| | 1-(6-(4-(5-chloro-6-methyl- | | |
| | 1H-indazol-4-yl)-3-(2,2- | | |
| | dimethyl-4-(methyl(oxetan- | | |
| | 3-yl)amino)piperidin-1-yl)- | | |
| | 5-methyl-1H-pyrazol-1-yl)- | | |
| | 2-azaspiro[3.3]heptan-2- | | |
| | yl)prop-2-en-1-one | | |
| 63a/63b | 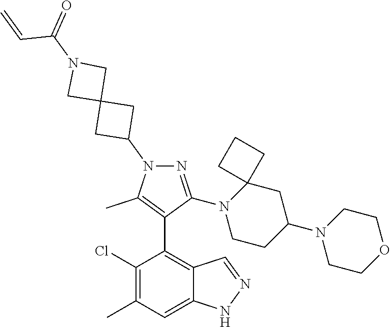 [SMILES: C=CC(=O)N1CC2(CC(n3nc(N4CCC(N5CCOCC5)CC45CCC5)c(-c4c(Cl)c(C)cc5[nH]ncc45)c3C)C2)C1] | Using Method- 4ek from Intermediate C103b (Step 2) and RP-HPLC-5 (mobile phase: A: 0.1% NH₃ in water/B: CH₃CN, gradient: 35 to 40% B in 30 min, 40% B for 17 min, 40 to 100% in 2 min, 100 to 35% in 6 min); Example 63a = 1^{st} eluting isomer, Example 63b = 2^{nd} eluting isomer | Example 63a: 1H NMR (400 MHz, Methanol-d₄) δ 7.72 (s, 1H), 7.45 (s, 1H), 6.38-6.34 (m, 1H), 6.29 (d, 1H), 5.78- 5.75 (d, 1H), 4.80 (m, 1H), 4.45 (s, 1H), 4.42 (s, 1H), 4.22 (s, 1H), 4.18 (s, 1H), 3.69-3.68 (m, 4H), 3.05-3.02 (m, 1H), 2.86- 2.78 (m, 4H), 2.66 (m, 1H), 2.63 (m, 6H), 2.45 (m, 1H), 2.39 (m, 1H), 2.06 (s, 3H), 2.02-1.89 (m, 6H), 1.76-1.70 (m, 2H), 1.53 (m, 1H), 1.31-1.27 (m, 1H); UPLC-MS-14: (mobile phase: A: [5 mM ammonium bicarbonate + 0.1% NH₃ in water]/[CH₃CN + 0.1% NH₃] gradient: 90:10 at 0.01 min, 70:30 at 10.0 min, 40:60 at 20.0 min, 0:100 at 26 min): Rt = 21.3 min, MS m/z [M + H]⁺ 604.2/ |
| | | | 606.2. Example 63b: UPLC- |
| | 1-(6-(4-(5-chloro-6-methyl- | | MS-14: (mobile phase: A: [5 |
| | 1H-indazol-4-yl)-5-methyl- | | mM ammonium bicarbonate + |
| | 3-(8-morpholino-5- | | 0.1% NH₃ in water]/[CH₃CN + |
| | azaspiro[3.5]nonan-5-yl)- | | 0.1% NH₃] gradient: 90:10 at |
| | 1H-pyrazol-1-yl)-2- | | 0.01 min, 70:30 at 10.0 min, |
| | azaspiro[3.3]heptan-2- | | 40:60 at 20.0 min, 0:100 at 26 |
| | yl)prop-2-en-1-one | | min): Rt = 27.3 min, MS m/z |
| | | | [M + H]⁺ 604.2/606.2. |
| 64 | 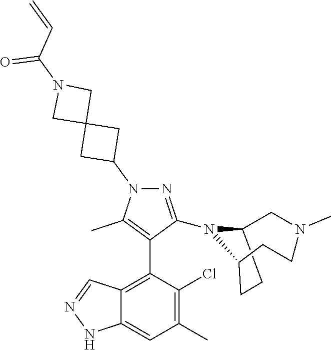 [SMILES: C=CC(=O)N1CC2(CC(n3nc(N4[C@H]5CC[C@@H]4CN(C)CC5)c(-c4c(Cl)c(C)cc5[nH]ncc45)c3C)C2)C1] | Using Method-4 from CAS [2307753-89-3] (Step 1) | Example 65: ¹H NMR (600 MHz, DMSO-d₆) δ 12.94-13.23 (m, 1H), 7.68-7.52 (m, 1H), 7.47 (s, 1H), 6.36-6.25 (m, 1H), 6.09 (m, 1H), 5.67 (m, 1H), 4.75-4.59 (m, 1H), 4.32 (s, 1H), 4.28 (s, 1H), 4.03 (s, 1H), 3.99 (s, 1H), 3.83-3.64 (m, 2H), 2.78-2.63 (m, 4H), 2.47 (s, 3H), 2.13-1.92 (m, 5H), 1.89 (s, 1.5H), 1.87 (s, 1.5H), 1.91-1.68 (m, 2H), 1.66-1.43 (m, 3H), 1.40-1.18 (m, 3H). UPLC-MS- 4: Rt = 0.63 min, MS m/z [M + H]⁺ 534.5/536.4 and Rt = 0.66 min, MS m/z [M + H]+ 534.4/536.5. |
| | 1-(6-(4-(5-chloro-6-methyl- | | |
| | 1H-indazol-4-yl)-5-methyl- | | |
| | 3-((1R,6S)-3-methyl-3,9- | | |
| | diazabicyclo[4.2.1]nonan- | | |
| | 9-yl)-1H-pyrazol-1-yl)-2- | | |
| | azaspiro[3.3]heptan-2- | | |
| | yl)prop-2-en-1-one | | |
| 65a/65b | 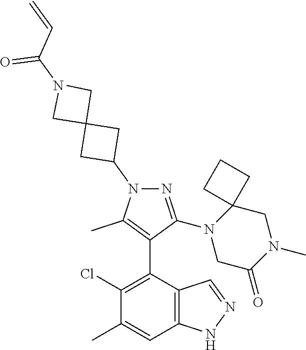 [SMILES: C=CC(=O)N1CC2(CC(n3nc(N4CC(=O)N(C)CC45CCC5)c(-c4c(Cl)c(C)cc5[nH]ncc45)c3C)C2)C1] | Using Method-4 from Intermediate A28 (Step 1) and C-HPLC-4 (mobile phase: [heptane + 0.1% DEA]/ [EtOH + 0.1% DEA] 40:60); Example 65a = 1^{st} eluting isomer, Example 65b = 2^{nd} eluting isomer | Example 65b: ¹H NMR (600 MHz, DMSO-d₆) δ 13.03 (s, 1H), 7.61 (s, 1H), 7.48 (s, 1H), 6.31 (m, 1H), 6.10 (m, 1H), 5.67 (m, 1H), 4.73 (m, 1H), 4.33 (s, 1H), 4.27 (s, 1H), 4.04 (s, 1H), 3.99 (s, 1H), 3.54 (m, 1H), 3.39 (m, 1H), 3.19 (m, 1H), 3.10 (m, 1H), 2.78 (s, 3H), 2.64-2.76 (m, 4H), 2.49 (s, 3H), 2.13 (m, 1H), 1.97 (s, 3H), 1.94 (m, 1H), 1.74- 1.82 (m, 2H), 1.71 (m, 1H), 1.63 (m, 1H). UPLC-MS-2a: Rt = 0.92 min, MS m/z [M + H]⁺ 548.4/ 550.4; C-HPLC-5 (mobile phase: [heptane + 0.1% DEA]/ [EtOH + 0.1% DEA] 40:60): Rt = 9.50 min. Example 65a: C- HPLC-5 (mobile phase: [heptane + 0.1% DEA] [EtOH + 0.1% DEA] 40:60): Rt = 7.90 min. |
| | 5-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-8-methyl-5,8- | | |
| | diazaspiro[3.5]nonan-7- | | |
| | one | | |
| 66a/66b | 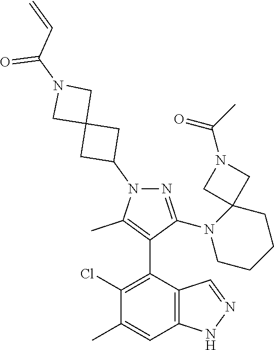 [SMILES: C=CC(=O)N1CC2(CC(n3nc(N4CCCCC45CN(C(C)=O)C5)c(-c4c(Cl)c(C)cc5[nH]ncc45)c3C)C2)C1] | Using Method-4e from Intermediate A29 (Step 1) and C-SFC-1 (mobile phase: [IPA + 0.1% Et₃N]/CO₂ 35:65); Example 66a = 1^{st} eluting isomer, Example 66b = 2^{nd} eluting isomer | Example 66b: ¹H NMR (400 MHz, DMSO-d₆) δ 13.03 (s, 1H), 7.58 (m, 1H), 7.47 (s, 1H), 6.30 (m, 1H), 6.09 (m, 1H), 5.67 (m, 1H), 4.72 (m, 1H), 4.33 (s, 1H), 4.27 (s, 1H), 4.21 (m, 0.5H), 4.05 (s, 1H), 4.04 (m, 0.5H), 3.97 (s, 1H), 3.93 (m, 0.5H), 3.88 (m, 1H), 3.80 (m, 0.5H), 3.55-3.64 (m, 1H), 2.64- 2.77 (m, 4H), 2.48 (s, 3H), 2.45 (m, 1H), 2.04 (m, 1H), 1.95 (s, 3H), 1.80 (s, 1.5H), 1.78 (s, 1.5H), 1.75 (m, 1H), 1.39-1.57 (m, 3H), 0.92-1.14 (m, 2H). UPLC-MS-2a: Rt = 0.99 min, MS m/z [M + H]⁺ 562.3/564.2; C-SFC-3 (mobile phase: [IPA + 0.1% Et₃N]/CO₂ 35:65): Rt = 2.42 min. Example 66a: C- SFC-3 (mobile phase: [IPA + 0.1% Et₃N]/CO₂ 35:65): Rt = 1.41 min. |
| | 1-(6-(3-(2-acetyl-2,5- | | |
| | diazaspiro[3.5]nonan-5-yl)- | | |
| | 4-(5-chloro-6-methyl-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |
| 67a/67b | 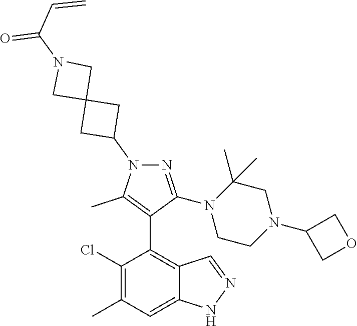 [SMILES: C=CC(=O)N1CC2(CC(n3nc(N4CCN(C5COC5)CC4(C)C)c(-c4c(Cl)c(C)cc5[nH]ncc45)c3C)C2)C1] | Using Method-4 from Intermediate A2 (Step 1) and C-SFC-1 (mobile phase: CO₂/[IPA + 0.1% Et₃N] 74:26); Example 67a = 1^{st} eluting isomer, Example 67b = 2^{nd} eluting isomer | Example 67b: ¹H NMR (600 MHz, DMSO-d₆) δ 12.9 (s, 1H), 7.57 (s, 1H), 7.44 (s, 1H), 6.31 (m, 1H), 6.10 (m, 1H), 5.67 (m, 1H), 4.74 (m, 1H), 4.43 (t, 1H), 4.40 (t, 1H), 4.33 (s, 1H), 4.29 (s, 1H), 4.31-4.26 (m, 2H), 4.04 (s, 1H), 4.01 (s, 1H), 3.12 (m, 1H), 2.90-2.79 (2H), 2.75-2.67 (m, 4H), 2.47 (s, 3H), 1.97 (s, 3H), 1.93 (m, 1H), 1.83-1.74 (m, 3H), 1.16-1.15 (m, 3H), 1.10-1.05 (m, 3H). UPLC-MS- 2e: Rt = 3.43 min; MS m/z [M + H]⁺ 564.3/566.3; C-SFC-3 (mobile phase: CO₂/[IPA + 0.1% NH₃] 74/26): Rt = 3.55 min, Example 67a: C-SFC-3 (mobile phase: CO₂/[IPA + 0.1% NH₃] 74/26): Rt = 2.32 min. |
| | 1-(6-(4-(5-chloro-6-methyl- | | |
| | 1H-indazol-4-yl)-3-(2,2- | | |
| | dimethyl-4-(oxetan-3- | | |
| | yl)piperazin-1-yl)-5-methyl- | | |
| | 1H-pyrazol-1-yl)-2- | | |
| | azaspiro[3.3]heptan-2- | | |
| | yl)prop-2-en-1-one | | |
| 68a/68b | 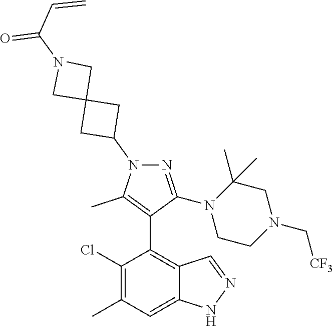 [SMILES: C=CC(=O)N1CC2(CC(n3nc(N4CCN(CC(F)(F)F)CC4(C)C)c(-c4c(Cl)c(C)cc5[nH]ncc45)c3C)C2)C1] | Using Method-4e from Intermediate A45 (Step 1) and C-SFC-4 (mobile phase: CO₂/IPA 82/18); Example 68a = 1^{st} eluting isomer, Example 68b = 2^{nd} eluting isomer | Example 68b: ¹H NMR (600 MHz, DMSO-d₆) δ 12.9 (s, 1H), 7.57 (s, 1H), 7.44 (s, 1H), 6.30 (m, 1H), 6.10 (m, 1H), 5.67 (m, 1H), 4.74 (m, 1H), 4.33 (s, 1H), 4.28 (s, 1H), 4.05 (s, 1H), 4.01 (s, 1H), 3.01-2.95 (m, 2H), 2.89-2.81 (m, 2H), 2.74-2.66 (m, 4H), 2.47 (s, 3H), 2.29 (m, 1H), 2.21-2.14 (m, 3H), 1.97 (s, 3H), 1.12-1.11 (m, 3H), 1.03- 1.02 (m, 3H). UPLC-MS-2e: Rt = 6.16 min; MS m/z [M + H]⁺ 590.3/592.3; C-SFC-3 (mobile phase: CO₂/IPA 82/18): Rt = 3.55 min, Example 68a: C- SFC-3 (mobile phase: CO₂/IPA 82/18): Rt = 3.03 min. |
| | 1-(6-(4-(5-chloro-6-methyl- | | |
| | 1H-indazol-4-yl)-3-(2,2- | | |
| | dimethyl-4-(2,2,2- | | |
| | trifluoroethyl)piperazin-1- | | |
| | yl)-5-methyl-1H-pyrazol-1- | | |
| | yl)-2-azaspiro[3.3]heptan- | | |
| | 2-yl)prop-2-en-1-one | | |
| 69a/69b | 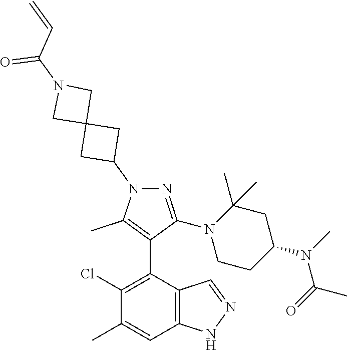 [SMILES: C=CC(=O)N1CC2(CC(n3nc(N4CC[C@@H](N(C)C(C)=O)CC4(C)C)c(-c4c(Cl)c(C)cc5[nH]ncc45)c3C)C2)C1] | Using Method-4 from Intermediate A54 (Step 1) and normal phase chromatography (eluent: MeOH in CH₂Cl₂ 0 to 10%); Example 69a = 1^{st} eluting isomer, Example 69b = 2^{nd} eluting isomer | Example 69b mixture of rotamers: ¹H NMR (600 MHz, DMSO-d₆) δ 13.0 (s, 1H), 7.58 (s, 1H), 7.46 (s, 1H), 6.30 (m, 1H), 6.10 (m, 1H), 5.67 (m, 1H), 4.76 (m, 1H), 4.60 (m, 0.5H), 4.34 (s, 1H), 4.29 (m, 1H), 4.06 (s, 1H), 4.00 (s, 1H), 3.83 (m, 0.5H), 3.30-3.16 (m, 1H), 2.92- 2.81 (m, 1H), 2.78-2.67 (m, 4H), 2.62 (s, 1.5H), 2.50 (s, 1.5H), 2.48 (s, 3H), 2.00 (s, 1.5H), 1.99 (s, 3H), 1.92 (s, 1.5H), 1.45 (m, 1H), 1.34-1.22 (m, 3H), 1.20-1-10 (m, 3H), 0.80-0.67 (m, 3H). UPLC-MS- 2a: Rt = 0.95 min; MS m/z [M + H]⁺ 578.4/580.4, Example 69a: UPLC-MS-2a: Rt = 0.93 min; MS m/z [M + H]⁺ 578.4/ 580.4. |
| | (R)-N-(1-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-2,2- | | |
| | dimethylpiperidin-4-yl)-N- | | |
| | methylacetamide | | |
| 70a/70b | 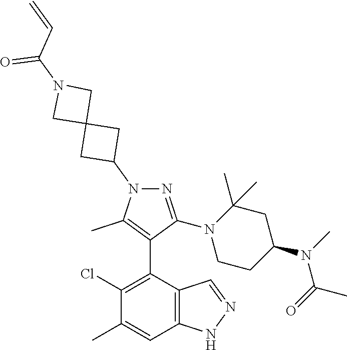 [SMILES: C=CC(=O)N1CC2(CC(n3nc(N4CC[C@H](N(C)C(C)=O)CC4(C)C)c(-c4c(Cl)c(C)cc5[nH]ncc45)c3C)C2)C1] | Using Method-4 from Intermediate A55 (Step 1) and normal phase chromatography (eluent: MeOH/CH₂Cl₂ (90/10) in CH₂Cl₂ 0 to 70%); Example 70a = 1^{st} eluting isomer, Example 70b = 2^{nd} eluting isomer | Example 70a mixture of rotamers: ¹H NMR (600 MHz, DMSO-d₆) δ 13.0 (s, 1H), 7.65 (s, 1H), 7.46 (s, 1H), 6.30 (m, 1H), 6.10 (m, 1H), 5.67 (m, 1H), 4.74 (m, 1H), 4.54 (m, 0.5H), 4.34 (s, 1H), 4.28 (m, 1H), 4.05 (s, 1H), 3.99 (s, 1H), 3.76 (m, 0.5H), 3.06-2.89 (m, 1H), 2.78- 2.61 (m, 5H), 2.56 (s, 1.5H), 2.49 (s, 3H), 2.45 (s, 1.5H), 1.99 (s, 3H), 1.97 (s, 1.5H), 1.90 (s, 1.5H), 1.38-1.28 (m, 4H), 1.23-1.17 (m, 2H), 1.16- 1.08 (m, 3H), 1.07-0.72 (m, 1H). UPLC-MS-2a: Rt = 0.95 min; MS m/z [M + H]⁺ 578.3/ 580.3, Example 70b: UPLC- MS-2a: Rt = 0.98 min; MS m/z [M + H]⁺ 578.3 / 580.3. |
| | (S)-N-(1-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-2,2- | | |
| | dimethylpiperidin-4-yl)-N- | | |
| | methylacetamide | | |
| 71a/71b | 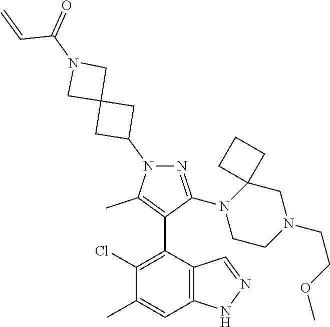 [SMILES: C=CC(=O)N1CC2(CC(n3nc(N4CCN(CCOC)CC45CCC5)c(-c4c(Cl)c(C)cc5[nH]ncc45)c3C)C2)C1] | Using Method-4 from Intermediate A27 (Step 1) and C-SFC-4 (mobile phase: CO₂/[IPA + 0.1% Et₃N] 75/25); Example 71a = 1^{st} eluting isomer, Example 71b = 2^{nd} eluting isomer | Example 71b: ¹H NMR (600 MHz, DMSO-d₆) δ 13.0 (s, 1H), 7.57 (s, 1H), 7.44 (s, 1H), 6.31 (m, 1H), 6.11 (m, 1H), 5.68 (m, 1H), 4.74 (m, 1H), 4.34 (s, 1H), 4.29 (m, 1H), 4.06 (s, 1H), 4.00 (m, 1H), 3.37 (m, 4H), 3.20 (s, 3H), 2.80-2.63 (m, 6H), 2.48 (s, 3H), 2.35 (m, 2H), 2.26-2.20 (m, 1H), 2.17-2.03 (m, 3H), 1.99 (s, 3H), 1.83-1.73 (m, 1H), 1.64-1.53 (m, 3H). UPLC-MS- 2e: Rt = 3.38 min; MS m/z [M + H]⁺ 578.4/580.4; C-SFC-3 (mobile phase: CO₂/[IPA + 0.1% Et₃N] 75/25): Rt = 3.06 min, Example 71a: C-SFC-3 (mobile phase: CO₂/[IPA + 0.1% Et₃N] 75/25): Rt = 2.26 min. |
| | 1-(6-(4-(5-chloro-6-methyl- | | |
| | 1H-indazol-4-yl)-3-(8-(2- | | |
| | methoxyethyl)-5,8- | | |
| | diazaspiro[3.5]nonan-5-yl)- | | |
| | 5-methyl-1H-pyrazol-1-yl)- | | |
| | 2-azaspiro[3.3]heptan-2- | | |
| | yl)prop-2-en-1-one | | |
| 72a/72b | 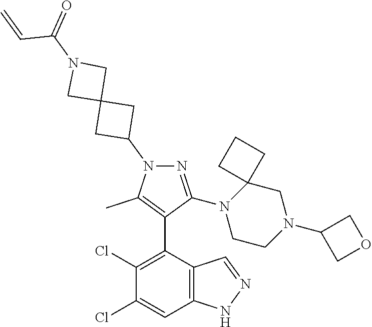 [SMILES: C=CC(=O)N1CC2(CC(n3nc(N4CCN(C5COC5)CC45CCC5)c(-c4c(Cl)c(Cl)cc5[nH]ncc45)c3C)C2)C1] | Using Method-4 from Intermediate A1 (Step 1) and D6 (Step 3) and C-SFC-4 (mobile phase: CO₂/[IPA + 0.1% Et₃N] 72/28); Example 72a = 1^{st} eluting isomer, Example 72b = 2^{nd} eluting isomer | Example 72b: ¹H NMR (600 MHz, DMSO-d₆) δ 13.2 (s, 1H), 7.78 (m, 1H), 7.67 (s, 1H), 6.31 (m, 1H), 6.10 (m, 1H), 5.67 (m, 1H), 4.80-4.72 (m, 1H), 4.48- 4.45 (m, 2H), 4.38-4.32 (m, 3H), 4.28 (m, 1H), 4.05 (s, 1H), 4.00 (m, 1H), 3.30-3.24 (m, 2H), 2.78-2.66 (m, 6H), 2.28- 2.16 (m, 2H), 2.13-2.05 (m, 2H), 2.01 (s, 3H), 1.95-1.89 (m, 1H), 1.83-1.74 (m, 1H), 1.62- 1.54 (m, 3H). UPLC-MS-2a: Rt = 0.86 min; MS m/z [M + H]⁺ 596.3/598.3; C-SFC-3 (mobile phase: CO₂/[IPA + 0.1% Et₃N] 72/28): Rt = 2.86 min, Example 72a: C-SFC-3 (mobile phase: CO₂/[IPA + 0.1% Et₃N] 72/28): Rt = 1.84 min. |
| | 1-(6-(4-(5,6-dichloro-1H- | | |
| | indazol-4-yl)-5-methyl-3- | | |
| | (8-(oxetan-3-yl)-5,8- | | |
| | diazaspiro[3.5]nonan-5-yl)- | | |
| | 1H-pyrazol-1-yl)-2- | | |
| | azaspiro[3.3]heptan-2- | | |
| | yl)prop-2-en-1-one1-yl)-2- | | |
| | azaspiro[3.3]heptan-2- | | |
| | yl)prop-2-en-1-one | | |
| 73a/73b | 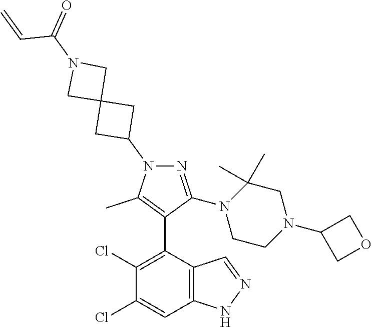 [SMILES: C=CC(=O)N1CC2(CC(n3nc(N4CCN(C5COC5)CC4(C)C)c(-c4c(Cl)c(Cl)cc5[nH]ncc45)c3C)C2)C1] | Using Method-4 from Intermediate A2 (Step 1) and D6 (Step 3) and C-SFC-4 (mobile phase: CO₂/[IPA + 0.1% Et₃N] 72/28); Example 73a = 1^{st} eluting isomer, Example 73b = 2^{nd} eluting isomer | Example 73b: ¹H NMR (600 MHz, DMSO-d₆) δ 13.2 (s, 1H), 7.79 (s, 1H), 7.67 (s, 1H), 6.30 (m, 1H), 6.10 (m, 1H), 5.67 (m, 1H), 4.75 (m, 1H), 4.45-4.40 (m, 2H), 4.35-4.27 (m, 4H), 4.05 (s, 1H), 4.01 (s, 1H), 3.14 (m, 1H), 2.92-2.84 (m, 2H), 2.77-2.67 (m, 4H), 2.00 (s, 3H), 1.98-1.90 (m, 1H), 1.90-1.63 (m, 3H), 1.14-1.09 (m, 3H), 1.09-0.96 (m, 3H). UPLC-MS- 2e: Rt = 3.72 min; MS m/z [M + H]⁺ 584.4/586.3; C-SFC-3 (mobile phase: CO₂/[IPA + 0.1% Et₃N] 72/28): Rt = 2.64 min, Example 73a: C-SFC-3 (mobile phase: CO₂/[IPA + 0.1% Et₃N] 72/28): Rt = 1.64 min. |
| | 1-(6-(4-(5,6-dichloro-1H- | | |
| | indazol-4-yl)-3-(2,2- | | |
| | dimethyl-4-(oxetan-3- | | |
| | yl)piperazin-1-yl)-5-methyl- | | |
| | 1H-pyrazol-1-yl)-2- | | |
| | azaspiro[3.3]heptan-2- | | |
| | yl)prop-2-en-1-one | | |
| 74a/74b | 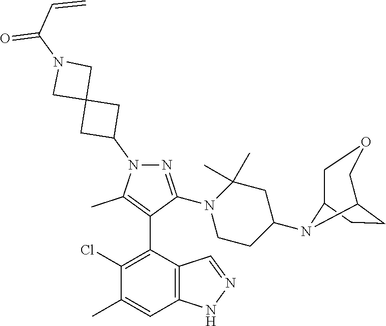 [SMILES: C=CC(=O)N1CC2(CC(n3nc(N4CCC(N5C6CCC5COC6)CC4(C)C)c(-c4c(Cl)c(C)cc5[nH]ncc45)c3C)C2)C1] | Using Method-4ai from Intermediate C97b (Step 2) and C-SFC-1 (mobile phase: CO₂/[IPA + 0.1% Et₃N] 70/30); Example 74a = 1^{st} eluting isomer, Example 74b = 2^{nd} eluting isomer | Example 74b: ¹H NMR (600 MHz, DMSO-d₆) δ 13.0 (s, 1H), 7.54 (s, 1H), 7.44 (s, 1H), 6.30 (m, 1H), 6.10 (m, 1H), 5.67 (m, 1H), 4.74 (m, 1H), 4.33 (s, 1H), 4.27 (m, 1H), 4.05 (s, 1H), 3.99 (s, 1H), 3.43-3.40 (m, 2H), 3.34-3.30 (m, 2H), 3.18-3.10 (m, 3H), 2.89-2.83 (m, 1H), 2.76-2.65 (m, 4H), 2.48 (s, 3H), 2.28-2.22 (m, 1H), 1.99 (s, 3H), 1.73-1.61 (m, 5H), 1.47 (m, 1H), 1.11-1.08 (m, 3H), 0.88- 0.80 (m, 1H), 0.76-0.70 (m, 4H). UPLC-MS-2e: Rt = 3.48 min; MS m/z [M + H]⁺ 618.3/ 620.4; C-SFC-3 (mobile phase: CO₂/[IPA + 0.025% NH₃] 70/30): Rt = 2.44 min, Example 74a: C-SFC-3 (mobile phase: CO₂/[IPA + 0.025% NH₃] 70/30): Rt = 1.73 min. |
| | 1-(6-(3-(4-((1R,5S)-3-oxa- | | |
| | 8-azabicyclo[3.2.1]octan- | | |
| | 8-yl)-2,2-dimethylpiperidin- | | |
| | 1-yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 75a/75b | 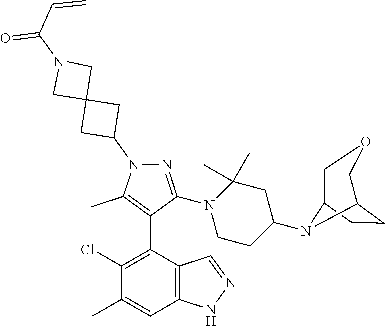 [SMILES: C=CC(=O)N1CC2(CC(n3nc(N4CCC(N5C6CCC5COC6)CC4(C)C)c(-c4c(Cl)c(C)cc5[nH]ncc45)c3C)C2)C1] | Using Method-4ai from Intermediate C97a (Step 2) and C-SFC-33 (mobile phase: CO₂/[IPA + 0.1% Et₃N] 70/30); Example 75a = 1^{st} eluting isomer, Example 75b = 2^{nd} eluting isomer | Example 75b: ¹H NMR (400 MHz, DMSO-d₆) δ 13.0 (s, 1H), 7.60 (s, 1H), 7.45 (s, 1H), 6.30 (m, 1H), 6.09 (m, 1H), 5.66 (m, 1H), 4.72 (m, 1H), 4.33 (s, 1H), 4.27 (s, 1H), 4.05 (s, 1H), 3.99 (s, 1H), 3.45-3.35 (m, 2H), 3.34-3.27 (m, 2H), 3.18-3.05 (m, 2H), 2.85-2.62 (m, 6H), 2.49 (s, 3H), 2.23-2.13 (m, 1H), 1.96-1.95 (m, 3H), 1.68-1.54 (m, 5H), 1.46-1.39 (m, 1H), 1.29 (s, 3H), 1.18-1.15 (m, 3H), 0.91 (m, 1H), 0.48-0.35 (m, 1H). UPLC-MS-2e: Rt = 3.22 min; MS m/z [M + H]⁺ 618.4/ 620.4; C-SFC-3 (mobile phase: CO₂/[IPA + 0.025% NH₃ v/v] 75/25): Rt = 4.31 min, Example 75a: C-SFC-3 (mobile phase: CO₂/[IPA + 0.025% NH₃ v/v] 75/25): Rt = 3.58 min. |
| | 1-(6-(3-(4-((1R,5S)-3-oxa- | | |
| | 8-azabicyclo[3.2.1]octan- | | |
| | 8-yl)-2,2-dimethylpiperidin- | | |
| | 1-yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 76a/76b | 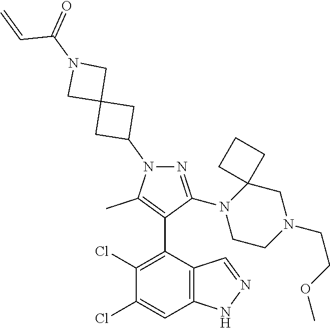 [SMILES: C=CC(=O)N1CC2(CC(n3nc(N4CCN(CCOC)CC45CCC5)c(-c4c(Cl)c(Cl)cc5[nH]ncc45)c3C)C2)C1] | Using Method-4f from Intermediate A27 (Step 1) and and D6 (Step 3) and C-SFC-4 (mobile phase: CO₂/[IPA + 0.1% Et₃N] 75/25); Example 76a = 1^{st} eluting isomer, Example 76b = 2^{nd} eluting isomer | Example 76b: ¹H NMR (400 MHz, DMSO-d₆) δ 13.2 (s, 1H), 7.78 (m, 1H), 7.67 (s, 1H), 6.30 (m, 1H), 6.09 (m, 1H), 5.67 (m, 1H), 4.76 (m, 1H), 4.34 (s, 1H), 4.28 (m, 1H), 4.05 (s, 1H), 4.00 (m, 1H), 3.37 (m, 2H), 3.19 (s, 3H), 2.79-2.64 (m, 6H), 2.44- 2.32 (m, 3H), 2.29-2.03 (m, 5H), 2.01 (s, 3H), 1.80-1.70 (m, 1H), 1.63-1.50 (m, 3H). UPLC- MS-2e: Rt = 3.53 min; MS m/z [M + H]⁺ 598.3/600.4; C-SFC-3 (mobile phase: CO₂/[IPA + 0.025% NH₃] 75/25): Rt = 3.17 min, Example 76a: C-SFC-3 (mobile phase: CO₂/[IPA + 0.025% NH₃] 75/25): Rt = 2.47 min. |
| | 1-(6-(4-(5,6-dichloro-1H- | | |
| | indazol-4-yl)-3-(8-(2- | | |
| | methoxyethyl)-5,8- | | |
| | diazaspiro[3.5]nonan-5-yl)- | | |
| | 5-methyl-1H-pyrazol-1-yl)- | | |
| | 2-azaspiro[3.3]heptan-2- | | |
| | yl)prop-2-en-1-one | | |
| 77 | 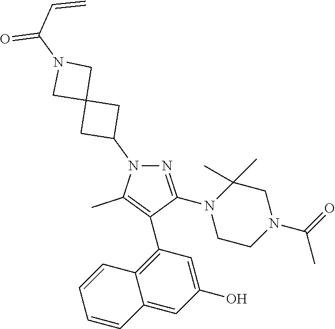 [SMILES: C=CC(=O)N1CC2(CC(n3nc(N4CCN(C(C)=O)CC4(C)C)c(-c4cc(O)cc5ccccc45)c3C)C2)C1] | Using Method- 4ceg from Intermediate A13 (Step 1) and intermediate D14 (Step 3) | Example 77: ¹H NMR (400 MHz, DMSO-d₆) δ 9.67 (s, 1H), 7.69 (d, 1H), 7.53 (d, 1H), 7.36 (t, 1H), 7.19 (t, 1H), 7.08 (m, 1H), 6.95-6.92 (m, 1H), 6.31 (m, 1H), 6.11 (m, 1H), 5.68 (m, 1H), 4.77 (m, 1H), 4.35 (s, 1H), 4.29 (m, 1H), 4.07 (s, 1H), 4.01 (m, 1H), 3.23-2.99 (m, 3H), 2.98-2.89 (m, 1H), 2.82-2.69 (m, 6H), 2.00 (s, 3H), 1.92 (s, 3H), 1.14-1.06 (m, 6H). UPLC- MS-2e: Rt = 4.24 min; MS m/z [M + H]⁺ 528.3. |
| | 1-(6-(3-(4-acetyl-2,2- | | |
| | dimethylpiperazin-1-yl)-4- | | |
| | (3-hydroxynaphthalen-1- | | |
| | yl)-5-methyl-1H-pyrazol-1- | | |
| | yl)-2-azaspiro[3.3]heptan- | | |
| | 2-yl)prop-2-en-1-one | | |
| 78a/78b | 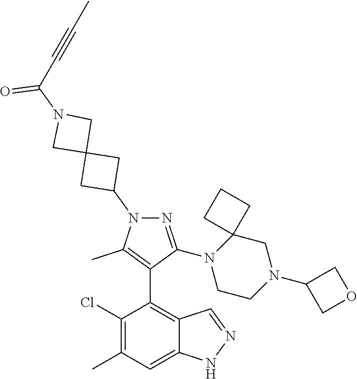 [SMILES: CC#CC(=O)N1CC2(CC(n3nc(N4CCN(C5COC5)CC45CCC5)c(-c4c(Cl)c(C)cc5[nH]ncc45)c3C)C2)C1] | Using Method-4 from Intermediate A1 (Step 1) and 2- butynoic acid (Step 5) and C- SFC-1 (mobile phase: CO₂/[IPA + 0.1% Et₃N] 72/28); Example 78a = 1^{st} eluting isomer, Example 78b = 2^{nd} eluting isomer | Example 78a: ¹H NMR (400 MHz, DMSO-d₆) δ 12.9 (s, 1H), 7.56 (s, 1H), 7.43 (s, 1H), 4.73 (m, 1H), 4.49-4.42 (m, 2H), 4.39-4.31 (m, 2H), 4.25 (s, 1H), 4.20 (m, 1H), 4.01 (s, 1H), 3.96 (m, 1H), 3.30-3.25 (m, 2H), 2.77-2.66 (m, 7H), 2.47 (s, 3H), 2.29-2.20 (m, 2H), 2.20-2.08 (m, 2H), 2.01-2.00 (s, 3H), 1.97 (s, 3H), 1.93-1.88 (m, 1H), 1.86-1.78 (m, 1H), 1.66-1.55 (m, 3H). UPLC-MS-2e: Rt = 3.70 min; MS m/z [M + H]⁺ 588.4/590.4; C-SFC-3 (mobile phase: CO₂/[IPA + 0.025% NH₃] 72/28): Rt = 2.02 min, Example 78b: C-SFC-3 (mobile phase: CO₂/[IPA + 0.025% NH₃] 72/28): Rt = 3.14 min. |
| | 1-(6-(4-(5-chloro-6-methyl- | | |
| | 1H-indazol-4-yl)-5-methyl- | | |
| | 3-(8-(oxetan-3-yl)-5,8- | | |
| | diazaspiro[3.5]nonan-5-yl)- | | |
| | 1H-pyrazol-1-yl)-2- | | |
| | azaspiro[3.3]heptan-2- | | |
| | yl)but-2-yn-1-one | | |
| 79a/79b | 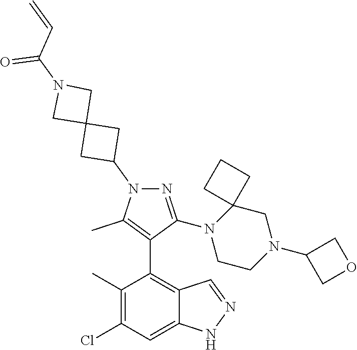 [SMILES: C=CC(=O)N1CC2(CC(n3nc(N4CCN(C5COC5)CC45CCC5)c(-c4c(C)c(Cl)cc5[nH]ncc45)c3C)C2)C1] | Using Method-4 from Intermediate A1 (Step 1) and Intermediate D10 (Step 3) and C- SFC-2 (mobile phase: CO₂/[IPA + 0.1% Et₃N] 70/30); Example 79a = 1^{st} eluting isomer, Example 79b = 2^{nd} eluting isomer | Example 79b: ¹H NMR (600 MHz, DMSO-d₆) δ 13.0 (s, 1H), 7.59 (s, 1H), 7.57 (s, 1H), 6.30 (m, 1H), 6.10 (m, 1H), 5.67 (m, 1H), 4.74 (m, 1H), 4.47 (m, 2H), 4.37-4.32 (m, 3H), 4.28 (m, 1H), 4.05 (m, 1H), 3.99 (m, 1H), 3.32-3.28 (m, 1H), 2.81-2.63 (m, 5H), 2.60-2.56 (m, 1H), 2.32-2.12 (m, 7H), 1.98-1.87 (m, 5H), 1.86-1.78 (m, 1H), 1.75-1.66 (m, 1H), 1.66-1.59 (m, 2H). UPLC-MS-2e: Rt = 3.88 min; MS m/z [M + H]⁺ 576.3/578.3; C-SFC-3 (mobile phase: CO₂/[IPA + 0.025% NH₃] 70/30): Rt = 1.97 min, Example 79a: C-SFC-3 (mobile phase: CO₂/[IPA + 0.025% NH₃] 70/30): Rt = 1.55 min. |
| | 1-(6-(4-(6-chloro-5-methyl- | | |
| | 1H-indazol-4-yl)-5-methyl- | | |
| | 3-(8-(oxetan-3-yl)-5,8- | | |
| | diazaspiro[3.5]nonan-5-yl)- | | |
| | 1H-pyrazol-1-yl)-2- | | |
| | azaspiro[3.3]heptan-2- | | |
| | yl)prop-2-en-1-one | | |
| 80a/80b | 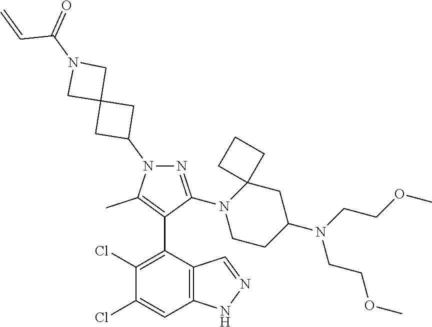 [SMILES: C=CC(=O)N1CC2(CC(n3nc(N4CCC(N(CCOC)CCOC)CC45CCC5)c(-c4c(Cl)c(Cl)cc5[nH]ncc45)c3C)C2)C1] | Using Method-4 from Intermediate C100a (Step 3) and D6 (Step 3) and C-SFC-35 (mobile phase: CO₂/[IPA + 0.1% Et₃N] 70/30); Example 80a = 1^{st} eluting isomer, Example 80b = 2^{nd} eluting isomer | Example 80b: ¹H NMR (600 MHz, DMSO-d₆) δ 13.2 (s, 1H), 7.79 (s, 1H), 7.71 (s, 1H), 6.30 (m, 1H), 6.10 (m, 1H), 5.67 (m, 1H), 4.74 (m, 1H), 4.33 (m, 1H), 4.27 (m, 1H), 4.04 (m, 1H), 3.98 (m, 1H), 3.26 (t, 4H), 3.20 (s, 6H), 2.79-2.64 (m, 4H), 2.58- 2.52 (m, 4H), 2.10 (m, 1H), 2.00 (s, 3H), 1.89-1.54 (m, 8H), 1.27-1.22 (m, 2H), 1.21-1.11 (m, 2H). UPLC-MS-4: Rt = 0.80 min; MS m/z [M + H]⁺ 670.5/ 672.4; C-SFC-3 (mobile phase: CO₂/[IPA + 0.025% NH₃] 65/35): Rt = 1.93 min, Example 80a: C-SFC-3 (mobile phase: CO₂/[IPA + 0.025% NH₃ v/v] 65/35): Rt = 1.11 min. |
| | 1-(6-(3-(8-(bis(2- | | |
| | methoxyethyl)amino)-5- | | |
| | azaspiro[3.5]nonan-5-yl)- | | |
| | 4-(5,6-dichloro-1H-indazol- | | |
| | 4-yl)-5-methyl-1H-pyrazol- | | |
| | 1-yl)-2- | | |
| | azaspiro[3.3]heptan-2- | | |
| | yl)prop-2-en-1-one | | |
| 81a/81b | 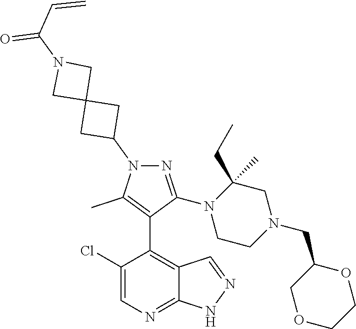 [SMILES: C=CC(=O)N1CC2(CC(n3nc(N4CCN(C[C@@H]5COCCO5)C[C@]4(C)CC)c(-c4c(Cl)cnc5[nH]ncc45)c3C)C2)C1] | Using Method-4 from intermediate C65 and intermediate D13 (Step 3) and C- SFC-7 (mobile phase: CO₂/[MeOH + 0.1% Et₃N 45/55); Example 81a = 1^{st} eluting isomer, Example 81b = 2^{nd} eluting isomer. | Example 81b: ¹H NMR (400 MHz, DMSO-d₆) δ 13.7 (s, 1H), 8.56 (s, 1H), 7.82 (s, 1H), 6.30 (m, 1H), 6.09 (m, 1H), 5.67 (m, 1H), 4.76 (m, 1H), 4.33 (s, 1H), 4.29 (s, 1H), 4.05 (s, 1H), 4.00 (s, 1H), 3.70-3.47 (m, 5H), 3.42-3.36 (m, 1H), 3.17 (m, 1H), 3.10-2.98 (m, 1H), 2.93- 2.83 (m, 1H), 2.78-2.62 (m, 4H), 2.24-2.07 (m, 5H), 2.04 (s, 3H), 1.90-1.73 (m, 2H), 1.61- 1.51 (m, 1H), 0.88-0.76 (m, 3H), 0.63 (m, 3H). UPLC-MS- 2e: Rt = 2.78 min; MS m/z [M + H]⁺ 609.5/611.4; C-SFC-8 (mobile phase: CO₂/[MeOH + 0.025% NH₃] 50/50): Rt = 3.39 min, Example 81a: C-SFC-8 (mobile phase: CO₂/[MeOH + 0.025% NH₃] 50/50): Rt = 1.96 min. |
| | 1-(6-(3-((S)-4-(((R)-1,4- | | |
| | dioxan-2-yl)methyl)-2- | | |
| | ethyl-2-methylpiperazin-1- | | |
| | yl)-4-(5-chloro-1H- | | |
| | pyrazolo[3,4-b]pyridin-4- | | |
| | yl)-5-methyl-1H-pyrazol-1- | | |
| | yl)-2-azaspiro[3.3]heptan- | | |
| | 2-yl)prop-2-en-1-one | | |
| 82a/82b | 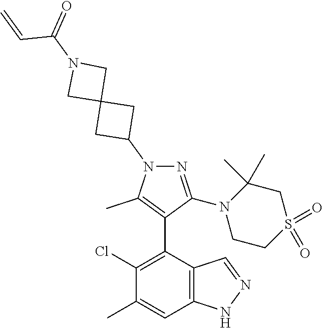 [SMILES: C=CC(=O)N1CC2(CC(n3nc(N4CCS(=O)(=O)CC4(C)C)c(-c4c(Cl)c(C)cc5[nH]ncc45)c3C)C2)C1] | Using Method- 4eh from 3,3- dimethylthio- morpholine 1,1-dioxide (Step 1) and C- SFC-1 (mobile phase: CO₂/IPA 70/30); Example 82a = 1^{st} eluting isomer, Example 82a = 2^{nd} eluting isomer | Example 82b: ¹H NMR (600 MHz, DMSO-d₆) δ 13.1 (s, 1H), 7.60 (s, 1H), 7.51 (s, 1H), 6.32 (dd, 1H), 6.11 (m, 1H), 5.68 (m, 1H), 4.82 (m, 1H), 4.35 (s, 1H), 4.32 (s, 1H), 4.05 (s, 1H), 4.00 (s, 1H), 3.42 (m, 2H), 3.09-2.93 (m, 4H), 2.81-2.75 (m, 2H), 2.72-2.66 (m, 2H), 2.50 (s, 3H), 1.98 (s, 3H), 1.13-1.07 (m, 6H); UPLC-MS-2a: Rt = 0.90 min; MS m/z [M + H]⁺ 557.3/559.3; C-SFC-3 (mobile phase: CO₂/IPA 70/30): Rt = 3.55 min, Example 82a: C-SFC-3 (mobile phase: CO₂/IPA 70/30): Rt = 2.82 min. |
| | 1-(6-(4-(5-chloro-6-methyl- | | |
| | 1H-indazol-4-yl)-3-(3,3- | | |
| | dimethyl-1,1- | | |
| | dioxidothiomorpholino)-5- | | |
| | methyl-1H-pyrazol-1-yl)-2- | | |
| | azaspiro[3.3]heptan-2- | | |
| | yl)prop-2-en-1-one | | |
| 83a/83b | 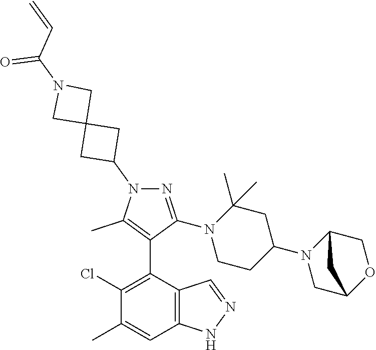 [SMILES: C=CC(=O)N1CC2(CC(n3nc(N4CCC(N5C[C@@H]6C[C@H]5CO6)CC4(C)C)c(-c4c(Cl)c(C)cc5[nH]ncc45)c3C)C2)C1] | Using Method- 4aeh from Intermediate C105a (Step 2) and C-SFC-4 (mobile phase: CO₂/[IPA + 0.025% NH₃]: 72/28): Example 83a = 1^{st} eluting isomer, Example 83b = 2^{nd} eluting isomer | Example 83b: ¹H NMR (400 MHz, DMSO-d₆) δ 12.9 (s, 1H), 7.54 (s, 1H), 7.44 (s, 1H), 6.30 (m, 1H), 6.10 (d, 1H), 5.67 (d, 1H), 4.74 (m, 1H), 4.33 (s, 1H), 4.28 (m, 1H), 4.23 (m, 1H), 4.05 (s, 1H), 4.00 (s, 1H), 3.85 (m, 1H), 3.51 (m, 1H), 3.41 (m, 1H), 3.19 (m, 1H), 2.88-2.65 (m, 6H), 2.48 (s, 3H), 2.14 (m, 1H), 2.00 (s, 3H), 1.62-1.41 (m, 4H), 1.14 (s, 3H), 0.94 (m, 1H), 0.77 (m, 1H), 0.71 (m, 3H); UPLC- MS-4: Rt = 0.74 min; MS m/z [M + H]⁺ 604.4/606.4; C-SFC-3 (mobile phase: CO₂/[IPA + 0.025% NH₃]: 72/28): Rt = 3.33 min, Example 83a: C-SFC-3 (mobile phase: CO₂/[IPA + 0.025% NH₃]: 72/28): Rt = 1.90 min. |
| | 1-(6-(3-(4-((1S,4S)-2-oxa- | | |
| | 5-azabicyclo[2.2.1]heptan- | | |
| | 5-yl)-2,2-dimethylpiperidin- | | |
| | 1-yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 84a/84b | 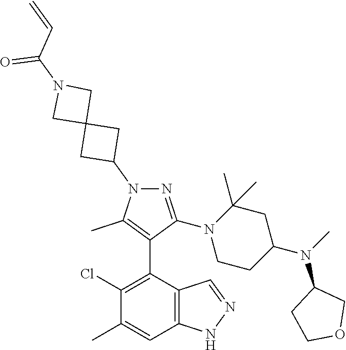 [SMILES: C=CC(=O)N1CC2(CC(n3nc(N4CCC(N(C)[C@@H]5CCOC5)CC4(C)C)c(-c4c(Cl)c(C)cc5[nH]ncc45)c3C)C2)C1] | Using Method- 4aeh from Intermediate C106a (Step 2) and C-SFC-4 (mobile phase: CO₂/[IPA + 0.1% Et₃N]: 72/28): Example 84a = 1^{st} eluting isomer, Example 84b = 2^{nd} eluting isomer | Example 84b: ¹H NMR (400 MHz, DMSO-d₆) δ 12.9 (s, 1H), 7.55 (s, 1H), 7.44 (s, 1H), 6.30 (m, 1H), 6.10 (d, 1H), 5.67 (d, 1H), 4.74 (m, 1H), 4.34 (s, 1H), 4.28 (m, 1H), 4.05 (s, 1H), 3.99 (s, 1H), 3.74 (m, 1H), 3.66 (t, 1H), 3.58 (dd, 1H), 3.38-3.27 (m, 1H), 3.23-3.07 (m, 2H), 2.83 (m, 1H), 2.75-2.61 (m, 5H), 2.48 (s, 3H), 2.00 (s, 3H), 1.95 (s, 3H), 1.93-1.83 (m, 1H), 1.65 (m, 1H), 1.43 (m, 1H), 1.30 (m, 1H), 1.20-1.02 (m, 2H), 1.11 (s, 3H), 0.78 (m, 3H); UPLC-MS-4: Rt = 0.73 min; MS m/z [M + H]⁺ 606.4/608.4; C- SFC-3 (mobile phase: CO₂/[IPA + 0.025% NH₃]: 70/30): Rt = 2.02 min, Example 84a: C-SFC-3 (mobile phase: CO₂/[IPA + 0.025% NH₃]: 70/30): Rt = 1.34 min. |
| | 1-(6-(4-(5-chloro-6-methyl- | | |
| | 1H-indazol-4-yl)-3-(2,2- | | |
| | dimethyl-4-(methyl((R)- | | |
| | tetrahydrofuran-3- | | |
| | yl)amino)piperidin-1-yl)-5- | | |
| | methyl-1H-pyrazol-1-yl)-2- | | |
| | azaspiro[3.3]heptan-2- | | |
| | yl)prop-2-en-1-one | | |
| 85a/85b | 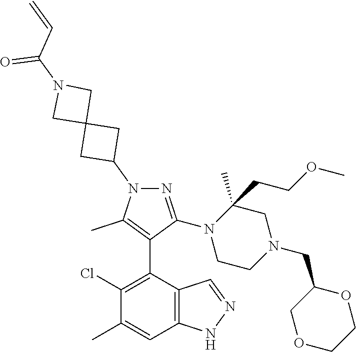 [SMILES: C=CC(=O)N1CC2(CC(n3nc(N4CCN(C[C@@H]5COCCO5)C[C@]4(C)CCOC)c(-c4c(Cl)c(C)cc5[nH]ncc45)c3C)C2)C1] | Using Method-4e from Intermediate C62 (Step 2) and C-HPLC-10 (mobile phase: [n- heptane/IPA] 80/20 + 0.05% Et₃N); Example 85a = 1^{st} eluting isomer, Example 85b = 2^{nd} eluting isomer | Example 85b: ¹H NMR (400 MHz, DMSO-d₆) δ 13.0 (s, 1H), 7.55 (s, 1H), 7.45 (s, 1H), 6.30 (m, 1H), 6.10 (m, 1H), 5.66 (m, 1H), 4.73 (m, 1H), 4.33 (s, 1H), 4.29 (s, 1H), 4.04 (s, 1H), 4.00 (s, 1H), 3.70-3.46 (m, 5H), 3.39 (td, 1H), 3.23 (m, 1H), 3.18- 3.11 (m, 5H), 3.02 (m, 1H), 2.80-2.63 (m, 5H), 2.47 (s, 3H), 2.22-1.83 (m, 8H), 1.96 (s, 3H), 0.90 (m, 3H). UPLC-MS-4: Rt = 0.67 min; MS m/z [M + H]⁺ 652.6/654.6; C-HPLC-9 (mobile phase: [n-heptane/IPA] 80/20 + 0.05% DEA): Rt = 6.94 min, Example 85a: C-HPLC-9 (mobile phase: [n-heptane/IPA] 80/20 + 0.05% DEA): Rt = 4.68 min. |
| | 1-(6-(3-((S)-4-(((R)-1,4- | | |
| | dioxan-2-yl)methyl)-2-(2- | | |
| | methoxyethyl)-2- | | |
| | methylpiperazin-1-yl)-4-(5- | | |
| | chloro-6-methyl-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |
| 86a/86b | 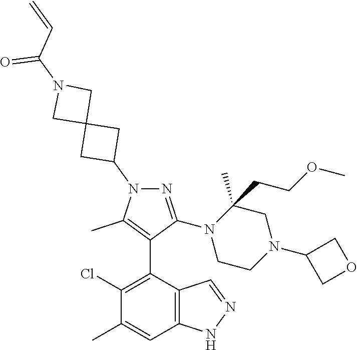 [SMILES: C=CC(=O)N1CC2(CC(n3nc(N4CCN(C5COC5)C[C@]4(C)CCOC)c(-c4c(Cl)c(C)cc5[nH]ncc45)c3C)C2)C1] | Using Method-4 from Intermediate C57 (Step 2) and C-SFC-34 (mobile phase: CO₂/IPA 70/30); Example 86a = 1^{st} eluting isomer, Example 86b = 2^{nd} eluting isomer | Example 86b: ¹H NMR (400 MHz, DMSO-d₆) δ 13.0 (s, 1H), 7.55 (s, 1H), 7.44 (s, 1H), 6.30 (m, 1H), 6.10 (m, 1H), 5.67 (m, 1H), 4.74 (m, 1H), 4.44 (m, 2H), 4.35-4.27 (m, 4H), 4.04 (s, 1H), 4.00 (s, 1H), 3.25 (m, 1H), 3.20- 3.03 (m, 6H), 2.85-2.62 (m, 5H), 2.47 (s, 3H), 2.14-1.94 (m, 4H), 1.98 (s, 3H), 1.87 (m, 1H), 1.66 (m, 1H), 0.92 (m, 3H). UPLC-MS-4: Rt = 0.75 min; MS m/z [M + H]⁺ 608.3/610.3; C- SFC-3 (mobile phase: CO₂/IPA 75/25): Rt = 4.01 min, Example 86a: C-SFC-3 (mobile phase: CO₂/IPA 75/25): Rt = 3.02 min. |
| | (S)-1-(6-(4-(5-chloro-6- | | |
| | methyl-1H-indazol-4-yl)-3- | | |
| | (2-(2-methoxyethyl)-2- | | |
| | methyl-4-(oxetan-3- | | |
| | yl)piperazin-1-yl)-5-methyl- | | |
| | 1H-pyrazol-1-yl)-2- | | |
| | azaspiro[3.3]heptan-2- | | |
| | yl)prop-2-en-1-one | | |
| 87a/87b | 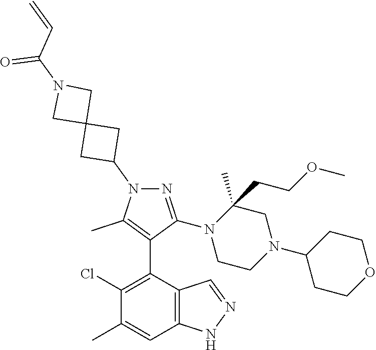 [SMILES: C=CC(=O)N1CC2(CC(n3nc(N4CCN(C5CCOCC5)C[C@]4(C)CCOC)c(-c4c(Cl)c(C)cc5[nH]ncc45)c3C)C2)C1] | Using Method- 4eh from Intermediate C58 (Step 2) and C- SFC-18 (mobile phase: CO₂/[MeOH + 0.1% Et₃N] 50/50); Example 87a = 1^{st} eluting isomer, Example 87b = 2^{nd} eluting isomer | Example 87a: ¹H NMR (400 MHz, DMSO-d₆) δ 12.9 (s, 1H), 7.55 (s, 1H), 7.44 (s, 1H), 6.30 (m, 1H), 6.10 (m, 1H), 5.66 (m, 1H), 4.73 (m, 1H), 4.33 (s, 1H), 4.29 (s, 1H), 4.04 (s, 1H), 4.00 (s, 1H), 3.81 (m, 2H), 3.27-3.10 (m, 7H), 3.03 (m, 1H), 2.81- 2.62 (m, 5H), 2.47 (s, 3H), 2.33- 1.88 (m, 7H), 1.96 (s, 3H), 1.56 (m, 2H), 1.27 (m, 2H), 0.91 (m, 3H). UPLC-MS-4: Rt = 0.65 min; MS m/z [M + H]⁺ 636.6/ 638.6; C-SFC-19 (mobile phase: CO₂/[MeOH + 0.025% NH3] 45/55): Rt = 2.65 min, Example 87b: C-SFC-19 (mobile phase: CO₂/[MeOH + 0.025% NH₃] 45/55): Rt = 4.61 min. |
| | (S)-1-(6-(4-(5-chloro-6- | | |
| | methyl-1H-indazol-4-yl)-3- | | |
| | (2-(2-methoxyethyl)-2- | | |
| | methyl-4-(tetrahydro-2H- | | |
| | pyran-4-yl)piperazin-1-yl)- | | |
| | 5-methyl-1H-pyrazol-1-yl)- | | |
| | 2-azaspiro[3.3]heptan-2- | | |
| | yl)prop-2-en-1-one | | |
| 88a/88b | 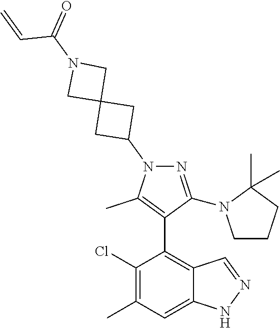 [SMILES: C=CC(=O)N1CC2(CC(n3nc(N4CCCC4(C)C)c(-c4c(Cl)c(C)cc5[nH]ncc45)c3C)C2)C1] | Using Method-4e from 2,2- dimethylpyrrolidine (Step 1) and C- SFC-1 (mobile phase: CO₂/[IPA + 0.1% Et₃N]: 77/23): Example 88a = 1^{st} eluting isomer, Example 88b = 2^{nd} eluting isomer | Example 88b: ¹H NMR (400 MHz, DMSO-d₆) δ 13.02 (s, 1H), 7.55 (s, 1H), 7.44 (s, 1H), 6.43-6.22 (m, 1H), 6.15-6.02 (m, 1H), 5.73-5.59 (m, 1H), 4.75-4.51 (m, 1H), 4.33 (s, 1H), 4.28 (s, 1H), 4.04 (s, 1H), 3.99 (s, 1H), 2.79-2.64 (m, 6H), 2.47 (s, 3H), 1.85 (s, 3H), 1.64-1.44 (m, 4H), 1.39 (s, 3H), 1.33 (s, 3H); UPLC-MS-4: Rt = 1.18 min; MS m/z [M + H]⁺ 493.3/ 495.3; C-SFC-3 (mobile phase: CO₂/[IPA + 0.025% NH₃] 75/25): Rt = 2.38 min, Example 88a: C- SFC-3 (mobile phase: CO₂/[IPA + 0.025% NH₃] 75/25): Rt = 1.80 min. |
| | 1-(6-(4-(5-chloro-6-methyl- | | |
| | 1H-indazol-4-yl)-3-(2,2- | | |
| | dimethylpyrrolidin-1-yl)-5- | | |
| | methyl-1H-pyrazol-1-yl)-2- | | |
| | azaspiro[3.3]heptan-2- | | |
| | yl)prop-2-en-1-one | | |
| 89a/89b | 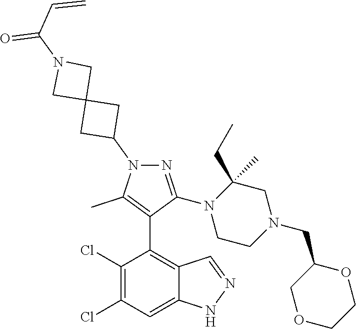 [SMILES: C=CC(=O)N1CC2(CC(n3nc(N4CCN(C[C@@H]5COCCO5)C[C@]4(C)CC)c(-c4c(Cl)c(Cl)cc5[nH]ncc45)c3C)C2)C1] | Using Method-4 from intermediate C65 and D6 (Step 3) (Step 3) and C- SFC-1 (mobile phase: CO₂/[IPA + 0.1% Et₃N] 72/28); Example 89a = 1^{st} eluting isomer, Example 89b = 2^{nd} eluting isomer. | Example 89b: UPLC-MS-2e: Rt = 3.49 min; MS m/z [M + H]⁺ 642.3/644.3/646.3; C-SFC-3 (mobile phase: CO₂/[IPA + 0.1% Et₃N] 72/28): Rt = 2.30 min, Example 89a: C-SFC-3 (mobile phase: CO₂/[IPA + 0.1% Et₃N] 72/28): Rt = 1.53 min. |
| | 1-(6-(3-((S)-4-(((R)-1,4- | | |
| | dioxan-2-yl)methyl)-2- | | |
| | ethyl-2-methylpiperazin-1- | | |
| | yl)-4-(5,6-dichloro-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |
| 90a/90b | 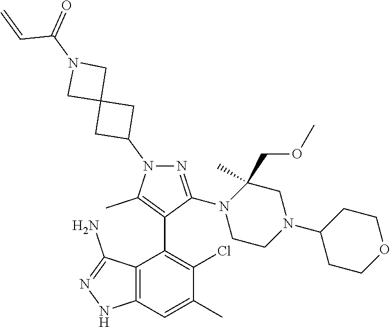 [SMILES: C=CC(=O)N1CC2(CC(n3nc(N4CCN(C5CCOCC5)C[C@]4(C)COC)c(-c4c(Cl)c(C)cc5[nH]nc(N)c45)c3C)C2)C1] | Using Method-4 from Intermediate C59 (Step 1) and C-SFC-34 (mobile phase: CO₂/[IPA + 0.1% Et₃N] 62/38); Example 90a = 1^{st} eluting isomer, Example 90b = 2^{nd} eluting isomer | Example 90a: UPLC-MS-2e: Rt = 2.92 min; MS m/z [M + H]⁺ 637.5/639.5; C-SFC-3 (mobile phase: CO₂/[IPA + 0.1% NH₃] 65/35): Rt = 1.76 min, Example 90b: C-SFC-3 (mobile phase: CO₂/[IPA + 0.1% NH₃] 65/35): Rt = 2.71 min. |
| | (R)-1-(6-(4-(3-amino-5- | | |
| | chloro-6-methyl-1H- | | |
| | indazol-4-yl)-3-(2- | | |
| | (methoxymethyl)-2-methyl- | | |
| | 4-(tetrahydro-2H-pyran-4- | | |
| | yl)piperazin-1-yl)-5-methyl- | | |
| | 1H-pyrazol-1-yl)-2- | | |
| | azaspiro[3.3]heptan-2- | | |
| | yl)prop-2-en-1-one | | |
| 91a/91b | 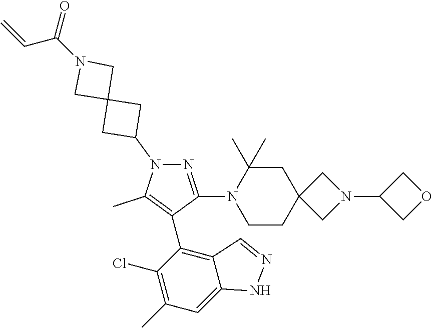 [SMILES: C=CC(=O)N1CC2(CC(n3nc(N4CCC5(CN(C6COC6)C5)CC4(C)C)c(-c4c(Cl)c(C)cc5[nH]ncc45)c3C)C2)C1] | Using Method-4 from Intermediate A67 (Step 1) and C-SFC-1 (mobile phase: CO₂/[IPA + 0.1% Et₃N]: 75/25): Example 91a = 1^{st} eluting isomer, Example 91b = 2^{nd} eluting isomer | Example 91b: ¹H NMR (600 MHz, DMSO-d₆) δ 12.95 (s, 1H), 7.56 (s, 1H), 7.44 (s, 1H), 6.39-6.20 (m, 1H), 6.16-6.03 (m, 1H), 5.73-5.58 (m, 1H), 4.83-4.61 (m, 1H), 4.53-4.44 (m, 2H), 4.33 (s, 1H), 4.31-4.25 (m, 3H), 4.05 (s, 1H), 3.98 (s, 1H), 3.66-3.58 (m, 1H), 3.05- 2.98 (m, 2H), 2.97-2.84 (m, 2H), 2.82-2.77 (m, 2H), 2.74- 2.66 (m, 4H), 2.48 (s, 3H), 1.97 (s, 3H), 1.50-1.38 (m, 2H), 1.30-1.22 (m, 2H), 1.00 (s, 1.5H), 0.99 (s, 1.5H), 0.91 (s, 1.5H), 0.90 (s, 1.5H); UPLC- MS-2a: Rt = 0.79 min; MS m/z [M + H]⁺ 604.6/606.6; C-SFC-3 (mobile phase: CO₂/[IPA + 0.025% NH₃] 75/25): Rt = 3.60 min, Example 91a: C- |
| | | | SFC-3 (mobile phase: |
| | 1-(6-(4-(5-chloro-6-methyl- | | CO₂/[IPA + 0.025% NH₃] 75/25): |
| | 1H-indazol-4-yl)-3-(6,6- | | Rt = 2.74 min. |
| | dimethyl-2-(oxetan-3-yl)- | | |
| | 2,7-diazaspiro[3.5]nonan- | | |
| | 7-yl)-5-methyl-1H-pyrazol- | | |
| | 1-yl)-2- | | |
| | azaspiro[3.3]heptan-2- | | |
| | yl)prop-2-en-1-one | | |
| 92a/92b | 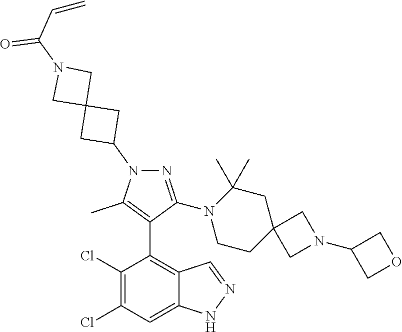 [SMILES: C=CC(=O)N1CC2(CC(n3nc(N4CCC5(CN(C6COC6)C5)CC4(C)C)c(-c4c(Cl)c(Cl)cc5[nH]ncc45)c3C)C2)C1] | Using Method-4 from Intermediate A67 (Step 1) and D6 (Step 3) and C-SFC-4 (mobile phase: CO₂/[IPA + 0.1% Et₃N] 75/25): Example 92a = 1^{st} eluting isomer, Example 92b = 2^{nd} eluting isomer | Example 92b: ¹H NMR (400 MHz, DMSO-d₆) δ 13.24 (s, 1H), 7.79 (s, 1H), 7.67 (s, 1H), 6.41-6.22 (m, 1H), 6.17-5.99 (m, 1H), 5.73-5.61 (m, 1H), 4.85-4.64 (m, 1H), 4.55-4.44 (m, 2H), 4.33 (s, 1H), 4.32-4.26 (m, 3H), 4.05 (s, 1H), 3.99 (s, 1H), 3.70-3.55 (m, 1H), 3.10- 2.88 (m, 4H), 2.85-2.77 (m, 2H), 2.76-2.67 (m, 4H), 2.00 (s, 3H), 1.54-1.43 (m, 2H), 1.37- 1.20 (m, 2H), 0.96 (s, 1.5H), 0.95 (s, 1.5H), 0.88 (m, 3H); UPLC-MS-4: Rt = 0.80 min; MS m/z [M + H]⁺ 624.4/626.4/ 628.4; C-SFC-3 (mobile phase: CO₂/[IPA + 0.025% NH₃] 75/25): Rt = 3.81 min, Example 92a: C- SFC-3 (mobile phase: CO₂/[IPA + 0.025% NH₃] 75/25): Rt = 2.95 min. |
| | 1-(6-(4-(5,6-dichloro-1H- | | |
| | indazol-4-yl)-3-(6,6- | | |
| | dimethyl-2-(oxetan-3-yl)- | | |
| | 2,7-diazaspiro[3.5]nonan- | | |
| | 7-yl)-5-methyl-1H-pyrazol- | | |
| | 1-yl)-2- | | |
| | azaspiro[3.3]heptan-2- | | |
| | yl)prop-2-en-1-one | | |
| 93a/93b | 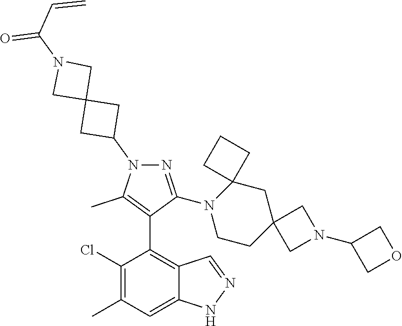 [SMILES: C=CC(=O)N1CC2(CC(n3nc(N4CCC5(CN(C6COC6)C5)CC45CCC5)c(-c4c(Cl)c(C)cc5[nH]ncc45)c3C)C2)C1] | Using Method-4 from Intermediate A68 (Step 1) and C-SFC-4 (mobile phase: CO₂/[IPA + 0.1% Et₃N] 72/28): Example 93a = 1^{st} eluting isomer, Example 93b = 2^{nd} eluting isomer | Example 93b: ¹H NMR (400 MHz, DMSO-d₆) δ 12.97 (s, 1H), 7.60 (s, 1H), 7.44 (s, 1H), 6.32-6.23 (m, 1H), 6.14-6.05 (m, 1H), 5.71-5.62 (m, 1H), 4.75-4.65 (m, 1H), 4.53-4.45 (m, 2H), 4.33-4.27 (m, 3H), 4.25 (s, 1H), 4.04 (s, 1H), 3.97 (s, 1H), 3.68-3.61 (m, 1H), 3.05-2.97 (m, 1H), 2.96-2.90 (m, 2H), 2.88-2.81 (m, 1H), 2.77-2.63 (m, 4H), 2.63-2.56 (m, 2H), 2.48 (s, 3H), 2.18-2.08 (m, 1H), 1.95 (s, 3H), 1.93-1.82 (m, 3H), 1.78-1.61 (m, 3H), 1.60-1.45 (m, 2H), 1.45-1.36 (m, 1H); UPLC-MS-4: Rt = 0.80 min; MS m/z [M + H]⁺ 616.5/ 618.5; C-SFC-3 (mobile phase: CO₂/[IPA + 0.025% NH₃] 72/28): Rt = 3.26 min, Example 93a: C- |
| | | | SFC-3 (mobile phase: |
| | 1-(6-(4-(5-chloro-6-methyl- | | CO₂/[IPA + 0.025% NH₃] 72/28): |
| | 1H-indazol-4-yl)-5-methyl- | | Rt = 2.33 min. |
| | 3-(2-(oxetan-3-yl)-2,10- | | |
| | diazadispiro[3.1.3⁶.3⁴]do- | | |
| | decan-10-yl)-1H-pyrazol-1- | | |
| | yl)-2-azaspiro[3.3]heptan- | | |
| | 2-yl)prop-2-en-1-one | | |
| 94a/94b | 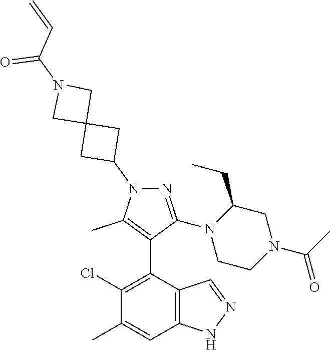 [SMILES: C=CC(=O)N1CC2(CC(n3nc(N4CCN(C(C)=O)C[C@@H]4CC)c(-c4c(Cl)c(C)cc5[nH]ncc45)c3C)C2)C1] | Using Method- 4be from Intermediate A14 (Step 1) and C- SFC-2 (mobile phase: CO₂/IPA 70/30); Example 94a = 1^{st} eluting isomer, Example 94b = 2^{nd} eluting isomer. | Example 94b: ¹H NMR (400 MHz, DMSO-d₆) δ 13.07 (s, 1H), 7.65 (m, 1H), 7.50 (s, 1H), 6.32 (m, 1H), 6.11 (m, 1H), 5.70 (m, 1H), 4.71 (m, 1H), 4.34 (s, 1H), 4.29 (s, 1H), 4.15 (d, 0.5H), 4.06 (s, 1H), 4.00 (s, 1H), 3.83 (d, 0.5H), 3.65 (m, 0.5H), 3.29 (m, 1H), 3.23 (m, 0.5H), 3.15 (m, 0.5H), 2.93 (m, 0.5H), 2.85-2.60 (m, 7H), 2.48 (s, 3H), 1.97 (s, 1.5H), 1.96 (s, 1.5H), 1.95 (s, 1.5H), 1.83 (s, 1.5H), 1.32-1.17 (m, 2H), 0.62- 0.47 (m, 3H). UPLC-MS-2e: Rt = 4.44; MS m/z [M + H]⁺ 550.4/ 552.4; C-SFC-3 (mobile phase: CO₂/IPA 70/30): Rt = 2.62 min, Example 94a: C-SFC-3 (mobile phase: CO₂/IPA 70/30): Rt = 1.67 min. |
| | (S)-1-(6-(3-(4-acetyl-2- | | |
| | ethylpiperazin-1-yl)-4-(5- | | |
| | chloro-6-methyl-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |
| 95a/95b | 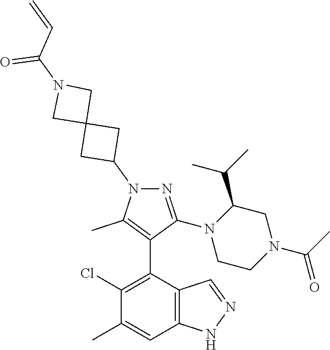 [SMILES: C=CC(=O)N1CC2(CC(n3nc(N4CCN(C(C)=O)C[C@@H]4C(C)C)c(-c4c(Cl)c(C)cc5[nH]ncc45)c3C)C2)C1] | Using Method- 4be from Intermediate A15 (Step 1) and C- SFC-1 (mobile phase: CO₂/[IPA + 0.1% Et₃N] 70/30); Example 95a = 1^{st} eluting isomer, Example 95b = 2^{nd} eluting isomer. | Example 95b: ¹H NMR (400 MHz, DMSO-d₆) δ 13.07 (s, 1H), 7.62 (s, 1H), 7.50 (s, 1H), 6.30 (m, 1H), 6.10 (m, 1H), 5.67 (m, 1H), 4.69 (m, 1H), 4.32 (s, 1H), 4.28 (s, 1H), 4.04 (s, 1H), 3.99 (s, 1H), 3.93 (m, 0.5H), 3.53 (m, 0.5H), 3.36 (m, 0.5H), 3.27-3.13 (m, 1.5H), 2.90 (m, 0.5H), 2.84-2.42 (m, 7.5H), 2.48 (s, 3H), 1.93 (s, 4.5H), 1.85 (s, 1.5H), 1.76 (m, 1H), 0.61 (m, 6H). UPLC-MS-2e: Rt = 4.80; MS m/z [M + H]⁺ 564.6/ 566.5; C-SFC-3 (mobile phase: CO₂/[IPA + 0.025% NH₃] 70/30): Rt = 2.43 min, Example 95a: C-SFC-3 (mobile phase: CO₂/[IPA + 0.025% NH₃] 70/30): Rt = 1.24 min. |
| | (S)-1-(6-(3-(4-acetyl-2- | | |
| | isopropylpiperazin-1-yl)-4- | | |
| | (5-chloro-6-methyl-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |
| 96a/96b | 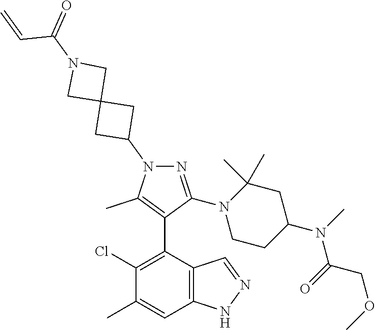 [SMILES: C=CC(=O)N1CC2(CC(n3nc(N4CCC(N(C)C(=O)COC)CC4(C)C)c(-c4c(Cl)c(C)cc5[nH]ncc45)c3C)C2)C1] | Using Method- 4be from Intermediate C82a (Step 2) and normal phase chromatography (eluent: CH₂Cl₂/[CH₂Cl₂ + 10% MeOH] 0 to 70%); Example 96a = 1^{st} eluting isomer, Example 96b = 2^{nd} eluting isomer. | Example 96b: ¹H NMR (400 MHz, DMSO-d₆) δ 12.97 (s, 1H), 7.57 (s, 1H), 7.46 (s, 1H), 6.31 (m, 1H), 6.10 (m, 1H), 5.67 (m, 1H), 4.75 (m, 1H), 4.57 (m, 0.5H), 4.34 (s, 1H), 4.28 (m 1H), 4.06-3.99 (m, 4H), 3.82 (m, 0.5H), 3.28-3.15 (m, 4H), 2.85 (m, 1H), 2.78-2.55 (m, 7H), 2.48 (s, 3H), 2.00 (s, 3H), 1.51-1.11 (m, 7H), 0.79 (m, 3H). UPLC-MS-2a: Rt = 0.94; MS m/z [M + H]⁺ 608.5/610.5, Example 96a: UPLC-MS-2a: Rt = 0.92; MS m/z [M + H]⁺ 608.5/610.5. |
| | N-(1-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-2,2- | | |
| | dimethylpiperidin-4-yl)-2- | | |
| | methoxy-N- | | |
| | methylacetamide | | |
| 97a/97b | 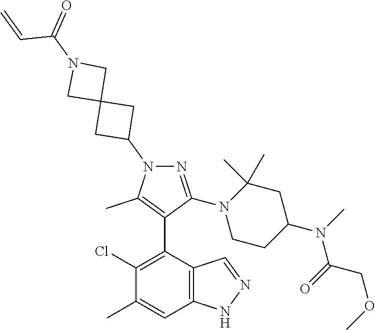 [SMILES: C=CC(=O)N1CC2(CC(n3nc(N4CCC(N(C)C(=O)COC)CC4(C)C)c(-c4c(Cl)c(C)cc5[nH]ncc45)c3C)C2)C1] | Using Method- 4be from Intermediate C82b (Step 2) and reverse phase HPLC (eluent: [H₂O + 5% TFA]/CH₃CN 5 to 60%); Example 97a = 1^{st} eluting isomer, Example 97b = 2^{nd} eluting isomer. | Example 97a: ¹H NMR (400 MHz, DMSO-d₆) δ 12.99 (s, 1H), 7.64 (s, 1H), 7.47 (s, 1H), 6.30 (m, 1H), 6.10 (m, 1H), 5.67 (m, 1H), 4.74 (m, 1H), 4.52 (m, 0.5H), 4.34 (s, 1H), 4.28 (s, 1H), 4.08-3.95 (m, 4H), 3.76 (m, 0.5H), 3.24 (m, 3H), 2.96 (m, 1H), 2.80-2.60 (m, 5H), 2.49 (s, 3H), 1.98 (s, 3H), 1.45 (m, 1H), 1.38-1.29 (m, 5H), 1.27-1.20 (m, 2H), 1.16-0.93 (m, 4H), 0.83 (m, 1H). UPLC- MS-2a: Rt = 0.92; MS m/z [M + H]⁺ 608.4/610.4, Example 97b: UPLC-MS-2a: Rt = 0.94; MS m/z [M + H]⁺ 608.5/610.5. |
| | N-(1-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-2,2- | | |
| | dimethylpiperidin-4-yl)-2- | | |
| | methoxy-N- | | |
| | methylacetamide | | |
| 98a/98b | 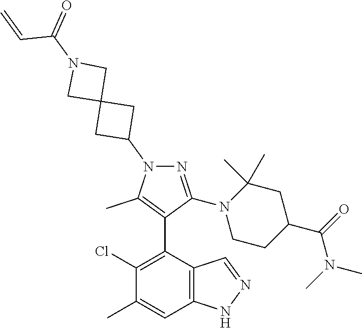 [SMILES: C=CC(=O)N1CC2(CC(n3nc(N4CCC(C(=O)N(C)C)CC4(C)C)c(-c4c(Cl)c(C)cc5[nH]ncc45)c3C)C2)C1] | Using Method-4ef from Intermediate C83a (Step 2) and normal phase chromatography (eluent: CH₂Cl₂/ [CH₂Cl₂ + 10% MeOH] 0 to 40%); Example 98a = 1^{st} eluting isomer, Example 98b = 2^{nd} eluting isomer. | Example 98b: ¹H NMR (400 MHz, DMSO-d₆) δ 12.96 (s, 1H), 7.53 (s, 1H), 7.44 (s, 1H), 6.31 (m, 1H), 6.10 (m, 1H), 5.67 (m, 1H), 4.75 (m, 1H), 4.33 (s, 1H), 4.28 (s, 1H), 4.05 (s, 1H), 4.00 (s, 1H), 3.21 (m, 1H), 2.96 (s, 3H), 2.88-2.77 (m, 2H), 2.75-2.66 (m, 7H), 2.48 (s, 3H), 1.99 (s, 3H), 1.28-1.22 (m, 4H), 1.18 (s, 6H). UPLC-MS-4: Rt; 0.96 min; MS m/z [M + H]⁺ 578.4/580.4; Example 98a: UPLC- MS-4: Rt; 0.94 min; MS m/z [M + H]⁺ 578.4/580.4. |
| | 1-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-N,N,2,2- | | |
| | tetramethylpiperidine-4- | | |
| | carboxamide | | |
| 99a/99b | 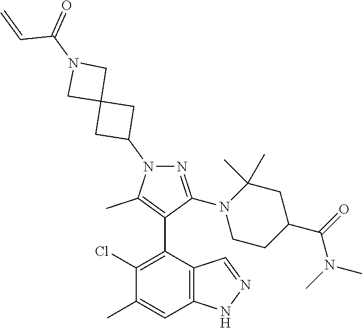 [SMILES: C=CC(=O)N1CC2(CC(n3nc(N4CCC(C(=O)N(C)C)CC4(C)C)c(-c4c(Cl)c(C)cc5[nH]ncc45)c3C)C2)C1] | Using Method-4ef from Intermediate C83b (Step 2) and RP-HPLC-1 (mobile phase: CH₃CN in [H₂O + 5% TFA] 5 to 60%); Example 99a = 1^{st} eluting isomer, Example 99b = 2^{nd} eluting isomer. | Example 99a: ¹H NMR (400 MHz, DMSO-d₆) δ 12.97 (s, 1H), 7.58 (s, 1H), 7.45 (s, 1H), 6.30 (m, 1H), 6.09 (m, 1H), 5.67 (m, 1H), 4.73 (m, 1H), 4.34 (s, 1H), 4.29 (s, 1H), 4.05 (s, 1H), 3.99 (s, 1H), 2.94 (s, 3H), 2.96- 2.88 (m, 1H), 2.81-2.62 (m, 6H), 2.72 (s, 3H), 2.48 (s, 3H), 1.95 (s, 3H), 1.38-1.32 (m, 6H), 1.28-1.16 (m, 2H), 0.94-0.71 (m, 2H). UPLC-MS-4: Rt = 0.95 min; MS m/z [M + H]⁺ 578.3/ 580.3; Example 99b: UPLC- MS-4: Rt = 0.96 min; MS m/z [M + H]⁺ 578.3/580.3. |
| | 1-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-N,N,2,2- | | |
| | tetramethylpiperidine-4- | | |
| | carboxamide | | |
| 100a/ 100b | 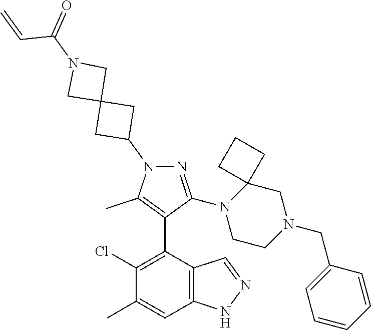 [SMILES: C=CC(=O)N1CC2(CC(n3nc(N4CCN(Cc5ccccc5)CC45CCC5)c(-c4c(Cl)c(C)cc5[nH]ncc45)c3C)C2)C1] | Using Method-4e from Intermediate A31 and C-SFC-4 (mobile phase: CO₂/[IPA + 0.025% NH₃] 70/30); Example 100a = 1^{st} eluting isomer, Example 100b = 2^{nd} eluting isomer | Example 100b: ¹H NMR (400 MHz, DMSO-d₆) δ 12.96 (s, 1H), 7.57 (s, 1H), 7.44 (s, 1H), 7.36-7.18 (m, 5H), 6.31 (m, 1H), 6.10 (m, 1H), 5.67 (m, 1H), 4.74 (m, 1H), 4.34 (s, 1H), 4.28 (s, 1H), 4.05 (s, 1H), 4.00 (s, 1H), 3.39 (d, 1H), 3.34 (d, 1H), 2.81-2.64 (m, 6H), 2.47 (s, 3H), 2.34 (m, 1H), 2.28-2.17 (m, 2H), 2.17-2.02 (m, 3H), 1.98 (s, 3H), 1.79 (m, 1H), 1.63-1.50 (m, 2H), 1.42 (m, 1H). UPLC- MS-2a: Rt = 0.86 min; MS m/z [M + H]⁺ 610.3/612.3; C-SFC-3 (mobile phase: CO₂/[IPA + 0.1% Et₃N] 70/30): Rt = 3.03 min, Example 100a: C-SFC-3 (mobile phase: CO₂/[IPA + 0.1% Et₃N] 70/30): Rt = 1.92 min. |
| | 1-(6-(3-(8-benzyl-5,8- | | |
| | diazaspiro[3.5]nonan-5-yl)- | | |
| | 4-(5-chloro-6-methyl-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |
| 101a/ 101b | 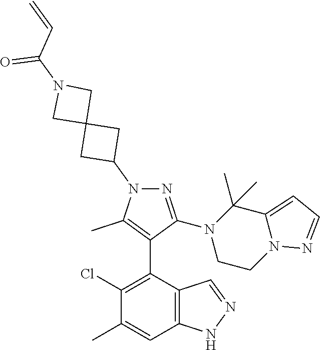 [SMILES: C=CC(=O)N1CC2(CC(n3nc(N4CCn5nccc5C4(C)C)c(-c4c(Cl)c(C)cc5[nH]ncc45)c3C)C2)C1] | Using Method- 4aeh from Intermediate A63 (Step 1) and C- SFC-1 (mobile phase: CO₂/IPA 67/33); Example 101a = 1^{st} eluting isomer, Example 101b = 2^{nd} eluting isomer | Example 101b: ¹H NMR (400 MHz, DMSO-d₆) δ 13.04 (s, 1H), 7.64 (s, 1H), 7.46 (s, 1H), 7.24 (m, 1H), 6.31 (m, 1H), 6.10 (m, 1H), 5.97 (d, 1H), 5.68 (m, 1H), 4.81 (m, 1H), 4.35 (s, 1H), 4.27 (s, 1H), 4.06 (s, 1H), 4.00 (s, 1H), 3.79-3.60 (m, 2H), 3.50-3.39 (m, 2H), 2.80-2.65 (m, 4H), 2.48 (s, 3H), 2.02 (s, 3H), 1.35 (s, 1.5H), 1.34 (s, 1.5H), 1.28 (s, 1.5H), 1.27 (s, 1.5H). UPLC-MS-2a: Rt = 0.97 min; MS m/z [M + H]⁺ 545.3/ 547.3; C-SFC-3 (mobile phase: CO₂/MeOH 65/35): Rt = 2.15 min, Example 101a: C-SFC-3 (mobile phase: CO₂/MeOH 65/35): Rt = 1.58 min. |
| | 1-(6-(4-(5-chloro-6-methyl- | | |
| | 1H-indazol-4-yl)-3-(4,4- | | |
| | dimethyl-6,7- | | |
| | dihydropyrazolo[1,5- | | |
| | a]pyrazin-5(4H)-yl)-5- | | |
| | methyl-1H-pyrazol-1-yl)-2- | | |
| | azaspiro[3.3]heptan-2- | | |
| | yl)prop-2-en-1-one | | |
| 102a/ 102b | 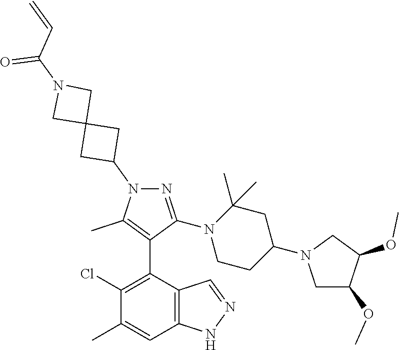 [SMILES: C=CC(=O)N1CC2(CC(n3nc(N4CCC(N5C[C@H](OC)[C@H](OC)C5)CC4(C)C)c(-c4c(Cl)c(C)cc5[nH]ncc45)c3C)C2)C1] | Using Method-4a from Intermediate C85a (Step 2) and C-SFC-4 (mobile phase: CO₂/[IPA + 0.1% Et₃N] 70/30); Example 102a = 1^{st} eluting isomer, Example 102b = 2^{nd} eluting isomer | Example 102b: ¹H NMR (400 MHz, DMSO-d₆) δ 12.9 (s, 1H), 7.54 (s, 1H), 7.44 (s, 1H), 6.30 (m, 1H), 6.10 (m, 1H), 5.67 (m, 1H), 4.74 (m, 1H), 4.33 (s, 1H), 4.28 (s, 1H), 4.05 (s, 1H), 4.00 (s, 1H), 3.66 (m, 2H), 3.23 (s, 3H), 3.22 (s, 3H), 3.13 (m, 1H), 2.86-2.78 (m, 3H), 2.75-2.65 (m, 4H), 2.48 (s, 3H), 2.28-2.24 (m, 3H), 2.00 (s, 3H), 1.62 (m, 1H), 1.45 (m, 1H) 1.12 (s, 3H), 1.00-0.89 (m, 1H), 0.87-0-78 (m, 1H), 0.75 (s, 1.5H), 0.74 (s, 1.5H). UPLC-MS-4: Rt = 0.78 min; MS m/z [M + H]⁺ 636.5/ 637.5; C-SFC-3 (mobile phase: CO₂/[IPA + 0.025% NH₃] 70/30): Rt = 2.86 min, Example 102a: C-SFC-3 (mobile phase: CO₂/[IPA + 0.025% NH₃] 70/30): Rt = 1.53 min. |
| | 1-(6-(4-(5-chloro-6-methyl- | | |
| | 1H-indazol-4-yl)-3-(4- | | |
| | ((3S,4R)-3,4- | | |
| | dimethoxypyrrolidin-1-yl)- | | |
| | 2,2-dimethylpiperidin-1-yl)- | | |
| | 5-methyl-1H-pyrazol-1-yl)- | | |
| | 2-azaspiro[3.3]heptan-2- | | |
| | yl)prop-2-en-1-one | | |
| 103a/ 103b | 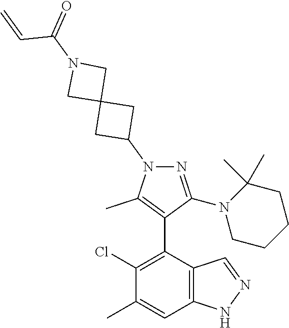 [SMILES: C=CC(=O)N1CC2(CC(n3nc(N4CCCCC4(C)C)c(-c4c(Cl)c(C)cc5[nH]ncc45)c3C)C2)C1] | Using Method-4b from 2,2- dimethylpiperidine (Step 1) and C- SFC-4 (mobile phase: CO₂/[IPA + 0.1% Et₃N] 75/25); Example 103a = 1^{st} eluting isomer, Example 103b = 2^{nd} eluting isomer | Example 103b: ¹H NMR (400 MHz, DMSO-d₆) δ 12.9 (s, 1H), 7.57 (s, 1H), 7.44 (s, 1H), 6.30 (m, 1H), 6.10 (m, 1H), 5.67 (m, 1H), 4.74 (m, 1H), 4.33 (s, 1H), 4.29 (s, 1H), 4.05 (s, 1H), 3.99 (s, 1H), 2.87-2.74 (m, 2H), 2.75-2.64 (m, 4H), 2.48 (s, 3H), 1.97 (s, 3H), 1.41-1.32 (m, 2H), 1.23-1.12 (m, 4H), 1.04 (s, 1.5H), 1.03 (s, 1.5H), 1.02 (s, 1.5H), 1.01 (s, 1.5H). UPLC- MS-2e: Rt = 5.72 min; MS m/z [M + H]⁺ 507.3/509.3; C-SFC-3 (mobile phase: CO₂/[IPA + 0.1% NH₃] 75/25): Rt = 3.80 min, Example 103a: C-SFC-3 (mobile phase: CO₂/[IPA + 0.1% NH₃] 75/25): Rt = 2.24 min. |
| | 1-(6-(4-(5-chloro-6-methyl- | | |
| | 1H-indazol-4-yl)-3-(2,2- | | |
| | dimethylpiperidin-1-yl)-5- | | |
| | methyl-1H-pyrazol-1-yl)-2- | | |
| | azaspiro[3.3]heptan-2- | | |
| | yl)prop-2-en-1-one | | |
| 104a/ 104b | 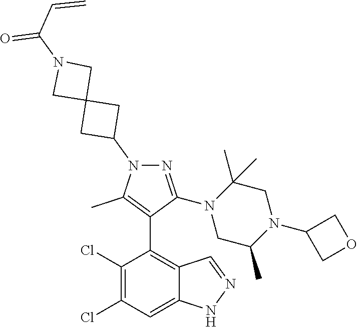 [SMILES: C=CC(=O)N1CC2(CC(n3nc(N4C[C@H](C)N(C5COC5)CC4(C)C)c(-c4c(Cl)c(Cl)cc5[nH]ncc45)c3C)C2)C1] | Using Method-4h from Intermediate C61 (Step 2) and D6 (Step 3) and C-SFC-4 (mobile phase: CO₂/IPA 72/28); Example 104a = 1^{st} eluting isomer, Example 104b = 2^{nd} eluting isomer | Example 104b: ¹H NMR (600 MHz, DMSO-d₆) δ 12.9 (s, 1H), 7.57 (s, 1H), 7.44 (s, 1H), 6.31 (m, 1H), 6.10 (m, 1H), 5.67 (m, 1H), 4.74 (m, 1H), 4.43 (t, 1H), 4.40 (t, 1H), 4.33 (s, 1H), 4.29 (s, 1H), 4.31-4.26 (m, 2H), 4.04 (s, 1H), 4.01 (s, 1H), 3.12 (m, 1H), 2.75-2.67 (m, 4H), 2.47 (s, 3H), 1.97 (s, 3H), 1.93 (m, 1H), 1.83-1.74 (m, 3H), 1.16-1.15 (m, 3H), 1.10-1.05 (m, 3H). UPLC-MS-2e: Rt = 3.70 min; MS m/z [M + H]⁺ 598.4/600.4; C-SFC-3 (mobile phase: CO₂/IPA 72/28): Rt = 3.08 min, Example 104a: C-SFC-3 (mobile phase: CO₂/IPA 72/28): Rt = 1.49 min. |
| | (S)-1-(6-(4-(5,6-dichloro- | | |
| | 1H-indazol-4-yl)-5-methyl- | | |
| | 3-(2,2,5-trimethyl-4- | | |
| | (oxetan-3-yl)piperazin-1- | | |
| | yl)-1H-pyrazol-1-yl)-2- | | |
| | azaspiro[3.3]heptan-2- | | |
| | yl)prop-2-en-1-one | | |
| 105a/ 105b | 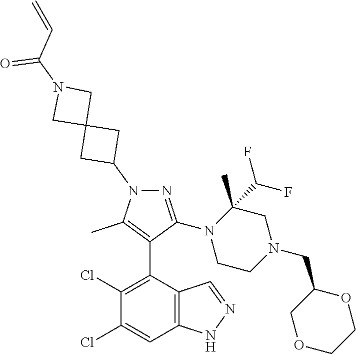 [SMILES: C=CC(=O)N1CC2(CC(n3nc(N4CCN(C[C@@H]5COCCO5)C[C@@]4(C)C(F)F)c(-c4c(Cl)c(Cl)cc5[nH]ncc45)c3C)C2)C1] | Using Method-4h from Intermediate C63 (Step 2) and D6 (Step 3) and C-SFC-4 (mobile phase: CO₂/[IPA + 0.1% Et₃N] 75/25); Example 105a = 1^{st} eluting isomer, Example 105b = 2^{nd} eluting isomer | Example 105b: ¹H NMR (400 MHz, DMSO-d₆) δ 13.29 (s, 1H), 7.82 (s, 1H), 7.69 (s, 1H), 6.63-6.33 (m, 1H), 6.33-6.24 (m, 1H), 6.14-6.02 (m, 1H), 5.74-5.62 (m, 1H), 4.84-4.66 (m, 1H), 4.33 (s, 1H), 4.29 (s, 1H), 4.05 (s, 1H), 4.00 (s, 1H), 3.73-3.44 (m, 6H), 3.43-3.35 (m, 1H), 3.19-3.11 (m, 2H), 2.85-2.64 (m, 6H), 2.36-2.25 (m, 1H), 2.19-2.11 (m, 2H), 1.98 (s, 3H), 1.95-1.89 (m, 1H), 0.99-0.94 (m, 3H). UPLC-MS- 2e: Rt = 3.99 min; MS m/z [M + H]⁺ 664.4/666.4/668.4; C- SFC-3 (mobile phase: CO₂/[IPA + 0.025% NH₃] 75/25): Rt = 2.88 min, Example 105a: C-SFC-3 (mobile phase: CO₂/[IPA + 0.025% NH₃] 75/25): Rt = 1.80 min. |
| | 1-(6-(3-((S)-4-(((R)-1,4- | | |
| | dioxan-2-yl)methyl)-2- | | |
| | (difluoromethyl)-2- | | |
| | methylpiperazin-1-yl)-4- | | |
| | (5,6-dichloro-1H-indazol-4- | | |
| | yl)-5-methyl-1H-pyrazol-1- | | |
| | yl)-2-azaspiro[3.3]heptan- | | |
| | 2-yl)prop-2-en-1-one | | |
| 106a/ 106b | 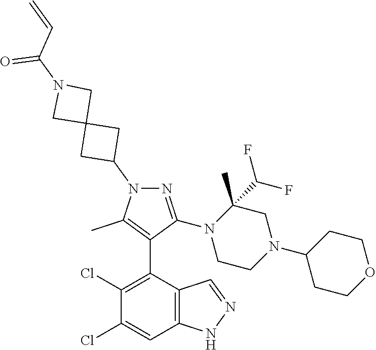 [SMILES: C=CC(=O)N1CC2(CC(n3nc(N4CCN(C5CCOCC5)C[C@@]4(C)C(F)F)c(-c4c(Cl)c(Cl)cc5[nH]ncc45)c3C)C2)C1] | Using Method-4h from Intermediate C60 (Step 2) and D6 (Step 3) and C-SFC-4 (mobile phase: CO₂/[IPA + 0.1% Et₃N] 70/30); Example 106a = 1^{st} eluting isomer, Example 106b = 2^{nd} eluting isomer | Example 106b: ¹H NMR (400 MHz, DMSO-d₆) δ 13.29 (s, 1H), 7.82 (s, 1H), 7.70 (s, 1H), 6.60-6.32 (m, 1H), 6.32-6.24 (m, 1H), 6.14-6.05 (m, 1H), 5.70-5.63 (m, 1H), 4.87-4.65 (m, 1H), 4.33 (s, 1H), 4.29 (s, 1H), 4.05 (s, 1H), 4.00 (s, 1H), 3.87-3.76 (m, 2H), 3.24-3.13 (m, 4H), 2.92-2.80 (m, 1H), 2.78-2.63 (m, 5H), 2.44-2.37 (m, 2H), 2.30-2.19 (m, 1H), 2.16-2.03 (m, 2H), 1.98 (s, 3H), 1.63-1.51 (m, 2H), 0.99-0.92 (m, 3H); UPLC-MS-2e: Rt = 3.58 min; MS m/z [M + H]⁺ 648.4/650.4/652.4; C-SFC-3 (mobile phase: CO₂/[IPA + 0.025% NH₃] 70/30): Rt = 2.24 min, Example 106a: C-SFC-3 (mobile phase: CO₂/[IPA + 0.025% NH₃] 70/30): Rt = 1.25 min. |
| | (S)-1-(6-(4-(5,6-dichloro- | | |
| | 1H-indazol-4-yl)-3-(2- | | |
| | (difluoromethyl)-2-methyl- | | |
| | 4-(tetrahydro-2H-pyran-4- | | |
| | yl)piperazin-1-yl)-5-methyl- | | |
| | 1H-pyrazol-1-yl)-2- | | |
| | azaspiro[3.3]heptan-2- | | |
| | yl)prop-2-en-1-one | | |
| 107a/ 107b | 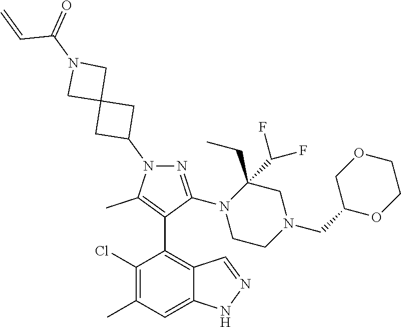 [SMILES: C=CC(=O)N1CC2(CC(n3nc(N4CCN(C[C@@H]5COCCO5)C[C@@]4(CC)C(F)F)c(-c4c(Cl)c(C)cc5[nH]ncc45)c3C)C2)C1] | Using Method-4h from Intermediate C64 (Step 2) and C-HPLC-10 (mobile phase: (n- heptane/IPA 60/40) + 0.05% Et₃N); Example 107a = 1^{st} eluting isomer, Example 107b = 2^{nd} eluting isomer | Example 107b: ¹H NMR (400 MHz, DMSO-d₆) δ 13.05 (s, 1H), 7.59 (s, 1H), 7.48 (s, 1H), 6.56 (m, 1H), 6.30 (m, 1H), 6.09 (m, 1H), 5.67 (m, 1H), 4.73 (m, 1H), 4.33 (s, 1H), 4.28 (s, 1H), 4.05 (s, 1H), 3.99 (s, 1H), 3.75- 3.35 (m, 6H), 3.18-3.09 (m, 2H), 2.85-2.59 (m, 6H), 2.49 (s, 3H), 2.30 (m, 1H), 2.21-2.09 (m, 2H), 1.97 (m, 1H), 1.92 (s, 3H), 1.79 (m, 1H), 1.69 (m, 2H), 0.62 (q, 3H). UPLC-MS-2e: Rt = 3.79 min; MS m/z [M + H]⁺ 658.5/660.4/662.4; C-HPLC- 9 (mobile phase: In- heptane + 0.05% DEA]/IPA 60/40): Rt = 3.91 min, Example 107a: C-HPLC-9 (mobile phase: [n-heptane + 0.05% DEA]/IPA 60/40): Rt = 1.82 min. |
| | 1-(6-(3-((S)-4-(((R)-1,4- | | |
| | dioxan-2-yl)methyl)-2- | | |
| | (difluoromethyl)-2- | | |
| | ethylpiperazin-1-yl)-4-(5- | | |
| | chloro-6-methyl-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |
| 108a/ 108b | 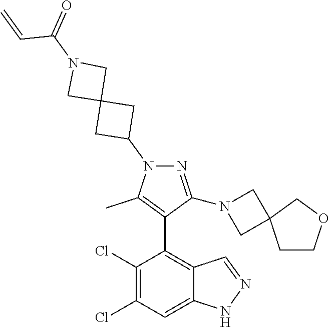 [SMILES: C=CC(=O)N1CC2(CC(n3nc(N4CC5(CCOC5)C4)c(-c4c(Cl)c(Cl)cc5[nH]ncc45)c3C)C2)C1] | Using Method-4f from 6-oxa-2- azaspiro[3.4]octane (Step 1) and D6 (Step 3) and C-SFC-1 (mobile phase: CO₂/[IPA + 0.05% Et₃N] 72/28); Example 108a = 1^{st} eluting isomer, Example 108b = 2^{nd} eluting isomer | Example 108b: ¹H NMR (600 MHz, DMSO-d₆) δ 13.35 (s, 1H), 7.81 (s, 1H), 7.75 (s, 1H), 6.30 (m, 1H), 6.10 (m, 1H), 5.67 (m, 1H), 4.70 (m, 1H), 4.33 (s, 1H), 4.27 (s, 1H), 4.05 (s, 1H), 3.98 (s, 1H), 3.60 (d, 1H), 3.57- 3.50 (m, 3H), 3.42-3.39 (m, 2H), 3.29-3.23 (m, 2H), 2.79- 2.60 (m, 4H), 1.96 (s, 3H), 1.94- 1.84 (m, 2H). UPLC-MS-4: Rt = 0.92 min; MS m/z [M + H]⁺ 527.3/529.3/531.3; C-SFC-3 (mobile phase: CO₂/[IPA + 0.025% NH₃] 70/30): Rt = 1.90 min, Example 108a: C-SFC-3 (mobile phase: CO₂/[IPA + 0.025% NH₃] 70/30): Rt = 1.45 min. |
| | 1-(6-(4-(5,6-dichloro-1H- | | |
| | indazol-4-yl)-5-methyl-3- | | |
| | (6-oxa-2- | | |
| | azaspiro[3.4]octan-2-yl)- | | |
| | 1H-pyrazol-1-yl)-2- | | |
| | azaspiro[3.3]heptan-2- | | |
| | yl)prop-2-en-1-one | | |
| 109a/ 109b | 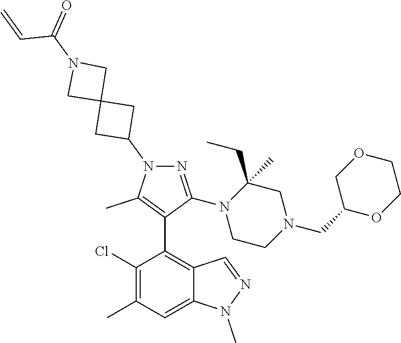 [SMILES: C=CC(=O)N1CC2(CC(n3nc(N4CCN(C[C@@H]5COCCO5)C[C@]4(C)CC)c(-c4c(Cl)c(C)cc5c4cnn5C)c3C)C2)C1] | Using Method-4 from Intermediate C65 (Step 3) and Intermediate D16 (Step 3) and C- SFC-44 (mobile phase: CO₂/[CH₃OH + 0.1% Et₃N] 58/42); Example 109a = 1^{st} eluting isomer, Example 109b = 2^{nd} eluting isomer | Example 109a: ¹H NMR (400 MHz, DMSO-d₆) δ 7.58 (s, 1H), 7.54 (s, 1H), 6.30 (m, 1H), 6.10 (m, 1H), 5.67 (m, 1H), 4.72 (m, 1H), 4.32 (s, 1H), 4.29 (s, 1H), 4.04 (s, 1H), 4.01 (s, 4H), 3.70- 3.46 (m, 5H), 3.39 (m, 1H), 3.16 (t, 1H), 2.98 (m, 1H), 2.80 (m, 1H), 2.76-2.63 (m, 5H), 2.48 (s, 3H), 2.14-2.05 (m, 4H), 1.94 (s, 3H), 1.90 (m, 1H), 1.78 (m, 1H), 1.63 (m, 1H), 0.92 (m, 3H), 0.64 (m, 3H). UPLC-MS-4: Rt = 0.76 min; MS m/z [M + H]⁺ 636.6/ 638.6/541.3; C-SFC-37 (mobile phase: CO₂/[CH₃OH + 0.025% NH₃] 58/42): Rt = 3.44 min, Example 109b: C-SFC-37 (mobile phase: CO₂/[CH₃OH + 0.025% NH₃] 58/42): Rt = 4.38 min. |
| | 1-(6-(3-((S)-4-(((R)-1,4- | | |
| | dioxan-2-yl)methyl)-2- | | |
| | ethyl-2-methylpiperazin-1- | | |
| | yl)-4-(5-chloro-1,6- | | |
| | dimethyl-1H-indazol-4-yl)- | | |
| | 5-methyl-1H-pyrazol-1-yl)- | | |
| | 2-azaspiro[3.3]heptan-2- | | |
| | yl)prop-2-en-1-one | | |
| 110a/ 110b | 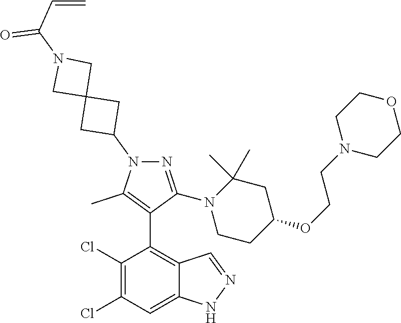 [SMILES: C=CC(=O)N1CC2(CC(n3nc(N4CC[C@@H](OCCN5CCOCC5)CC4(C)C)c(-c4c(Cl)c(Cl)cc5[nH]ncc45)c3C)C2)C1] | Using Method-4 from Intermediate C77 (Step 2) and D6 (Step 3) and C-SFC-38 (mobile phase: CO₂/[CH₃OH + 0.025% NH₃] 65/35); Example 110a = 1^{st} eluting isomer, Example 110b = 2^{nd} eluting isomer | Example 110b: UPLC-MS-2e: Rt = 3.95 min; MS m/z [M + H]⁺ 656.3/658.3/660.3; C-SFC- 39 (mobile phase: CO₂/[IPA + 0.05% Et₃N] 70/30): Rt = 3.42 min, Example 110a: C-SFC-39 (mobile phase: CO₂/[IPA + 0.05% Et₃N] 70/30): Rt = 1.72 min. |
| | (R)-1-(6-(4-(5,6-dichloro- | | |
| | 1H-indazol-4-yl)-3-(2,2- | | |
| | dimethyl-4-(2- | | |
| | morpholinoethoxy)piperi- | | |
| | din-1-yl)-5-methyl-1H- | | |
| | pyrazol-1-yl)-2- | | |
| | azaspiro[3.3]heptan-2- | | |
| | yl)prop-2-en-1-one | | |
| 111 | 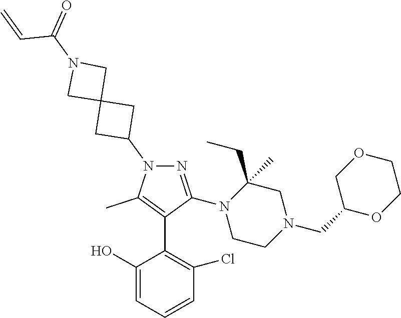 [SMILES: C=CC(=O)N1CC2(CC(n3nc(N4CCN(C[C@@H]5COCCO5)C[C@]4(C)CC)c(-c4c(O)cccc4Cl)c3C)C2)C1] | Using Method-4j from Intermediate C65 (Step 2) and (2-chloro-6- hydroxyphenyl) boronic acid (Step 3) and normal phase chromatography (eluent: MeOH in [CH₂Cl₂/MeOH: 80/20] from 0 to 50%) | Example 111: UPLC-MS-4: Rt = 0.74-0.76 min; MS m/z [M + H]⁺ 584.5/586.5. |
| | 1-(6-(3-((S)-4-(((R)-1,4- | | |
| | dioxan-2-yl)methyl)-2- | | |
| | ethyl-2-methylpiperazin-1- | | |
| | yl)-4-(2-chloro-6- | | |
| | hydroxyphenyl)-5-methyl- | | |
| | 1H-pyrazol-1-yl)-2- | | |
| | azaspiro[3.3]heptan-2- | | |
| | yl)prop-2-en-1-one | | |
| 112a/ 112b | 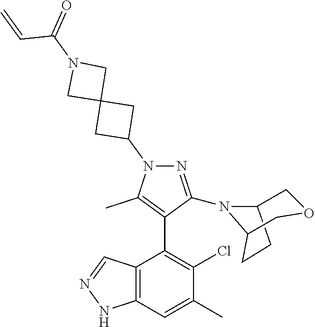 [SMILES: C=CC(=O)N1CC2(CC(n3nc(N4C5CCC4COC5)c(-c4c(Cl)c(C)cc5[nH]ncc45)c3C)C2)C1] | Using Method- 4bc from 3-oxa-8- azabicyclo[3.2.1] octane (CAS [280- 07-9]) (Step 1) and C-SFC-1 (mobile phase: CO₂/[IPA + 0.1% Et₃N] 70/30); Example 112a = 1^{st} eluting isomer, Example 112b = 2^{nd} eluting isomer | Example 112b: UPLC-MS-2e: Rt = 4.57 min; MS m/z [M + H]⁺ 507.4/509.4; C-SFC3 (mobile phase: CO₂/[IPA + 0.1%Et₃N] 70/30): Rt = 2.78 min, Example 112a: C-SFC-3 (mobile phase: CO₂/[IPA + 0.1%Et₃N] 70/30): Rt = 1.57 min. |
| | 1-(6-(3-((1R,5S)-3-oxa-8- | | |
| | azabicyclo[3.2.1]octan-8- | | |
| | yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 113a/ 113b | 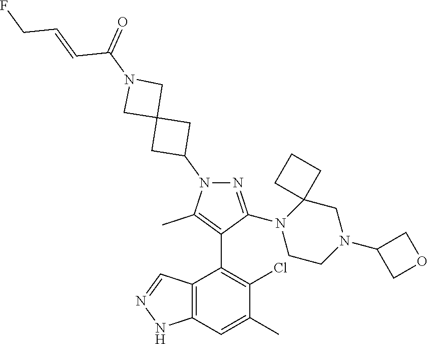 [SMILES: Cc1cc2[nH]ncc2c(-c2c(N3CCN(C4COC4)CC34CCC4)nn(C3CC4(C3)CN(C(=O)/C=C/CF)C4)c2C)c1Cl] | Using Method-4j from Intermediate A1 and (E)-4- fluorobut-2-enoic acid (CAS [37759- 72-1]) (Step 5) and C-SFC-1 (mobile phase: CO₂/[IPA + 0.1% NH₃] 72/28); Example 113a = 1^{st} eluting isomer, Example 113b = 2^{nd} eluting isomer | Example 113b: UPLC-MS-2d: Rt = 3.60 min; MS m/z [M + H]⁺ 608.4/610.4; C-SFC-3 (mobile phase: CO₂/[IPA + 0.1%NH₃] 70/30): Rt = 2.27 min, Example 113a: C-SFC-3 (mobile phase: CO₂/[IPA + 0.1%NH₃] 70/30): Rt = 1.61 min. |
| | (E)-1-(6-(4-(5-chloro-6- | | |
| | methyl-1H-indazol-4-yl)-5- | | |
| | methyl-3-(8-(oxetan-3-yl)- | | |
| | 5,8-diazaspiro[3.5]nonan- | | |
| | 5-yl)-1H-pyrazol-1-yl)-2- | | |
| | azaspiro[3.3]heptan-2-yl)- | | |
| | 4-fluorobut-2-en-1-one | | |
| 114a/ 114b | 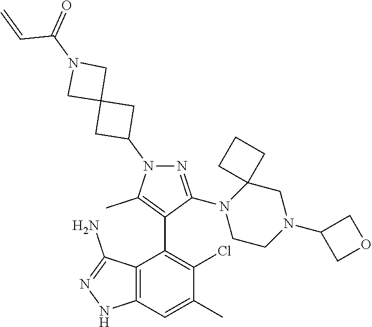 [SMILES: C=CC(=O)N1CC2(CC(n3nc(N4CCN(C5COC5)CC45CCC5)c(-c4c(Cl)c(C)cc5[nH]nc(N)c45)c3C)C2)C1] | Using Method-4 from Intermediate A1 (Step 1) and Intermediate D11 (Step 3) and C- SFC-7 (mobile phase: CO₂/[MeOH + 0.1%Et₃N] 55/45); Example 114a = 1^{st} eluting isomer, Example 114b = 2^{nd} eluting isomer | Example 114a: UPLC-MS-2e: Rt = 3.34 min; MS m/z [M + H]⁺ 591.3/593.3; C-SFC-8 (mobile phase: CO₂/[MeOH + 0.1%NH₃] 55/45): Rt = 2.64 min, Example 114b: C-SFC-8 (mobile phase: CO₂/[MeOH + 0.1%NH₃] 55/45): Rt = 3.72 min. |
| | 1-(6-(4-(3-amino-5-chloro- | | |
| | 6-methyl-1H-indazol-4-yl)- | | |
| | 5-methyl-3-(8-(oxetan-3- | | |
| | yl)-5,8- | | |
| | diazaspiro[3.5]nonan-5-yl)- | | |
| | 1H-pyrazol-1-yl)-2- | | |
| | azaspiro[3.3]heptan-2- | | |
| | yl)prop-2-en-1-one | | |
| 115a/ 115b | 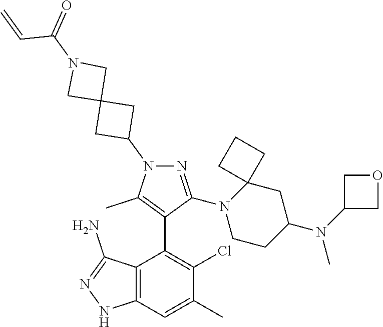 [SMILES: C=CC(=O)N1CC2(CC(n3nc(N4CCC(N(C)C5COC5)CC45CCC5)c(-c4c(Cl)c(C)cc5[nH]nc(N)c45)c3C)C2)C1] | Using Method-4 from Intermediate C101a + b (Step 3) and Intermediate D11 (Step 3) and normal phase chromatography to separate (eluent; MeOH in CH₂Cl₂ 0 to 20%) to give a fraction A as the first eluting mixture of isomer and fraction B as the second eluting mixture of isomers. Fraction A was further purified by C- SFC-4 (mobile phase: | Example 115a: UPLC-MS-4: Rt = 0.66 min; MS m/z [M + H]⁺ 619.5/621.5; C-SFC-3 (mobile phase: CO₂/[IPA + 0.1% NH₃] 65/35): Rt = 2.30 min, Example 115b: C-SFC-3 (mobile phase: CO₂/[IPA + 0.1% NH₃] 65/35): Rt = 3.92 min. |
| | | CO₂/[IPA + 0.1% | |
| | 1-(6-(4-(3-amino-5-chloro- | Et₃N] 60/40); | |
| | 6-methyl-1H-indazol-4-yl)- | Example 115a = | |
| | 5-methyl-3-(8- | 1^{st} eluting isomer, | |
| | (methyl(oxetan-3- | Example 115b = | |
| | yl)amino)-5- | 2^{nd} eluting isomer | |
| | azaspiro[3.5]nonan-5-yl)- | | |
| | 1H-pyrazol-1-yl)-2- | | |
| | azaspiro[3.3]heptan-2- | | |
| | yl)prop-2-en-1-one | | |
| 116a/ 116b | 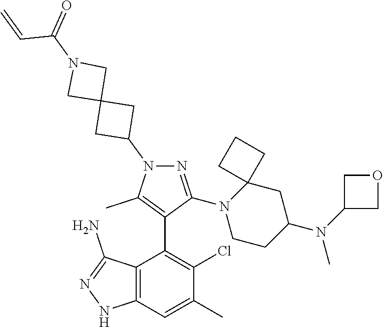 [SMILES: C=CC(=O)N1CC2(CC(n3nc(N4CCC(N(C)C5COC5)CC45CCC5)c(-c4c(Cl)c(C)cc5[nH]nc(N)c45)c3C)C2)C1] | Using Method-4 from Intermediate C101a + b (Step 3) and Intermediate D11 (Step 3) and normal phase chromatography to separate (eluent; MeOH in CH₂Cl₂ 0 to 20%) to give a fraction A as the first eluting mixture of isomers and fraction B as the second eluting mixture of isomers. Fraction B was further purified by C- SFC-7 (mobile | Example 116a: UPLC-MS-2e: Rt = 3.68 min; MS m/z [M + H]⁺ 619.5/621.4; C-SFC-8 (mobile phase: CO₂/[MeOH + 0.1% NH₃] 50/50): Rt = 2.75 min, Example 116b: C-SFC-8 (mobile phase: CO₂/[MeOH + 0.1% NH₃] 50/50): Rt = 3.97 min. |
| | | phase: | |
| | 1-(6-(4-(3-amino-5-chloro- | CO₂/[MeOH + | |
| | 6-methyl-1H-indazol-4-yl)- | 0.1% Et₃N] 50/50); | |
| | 5-methyl-3-(8- | Example 116a = | |
| | (methyl(oxetan-3- | 1^{st} eluting isomer, | |
| | yl)amino)-5- | Example 116b = | |
| | azaspiro[3.5]nonan-5-yl)- | 2^{nd} eluting isomer | |
| | 1H-pyrazol-1-yl)-2- | | |
| | azaspiro[3.3]heptan-2- | | |
| | yl)prop-2-en-1-one | | |
| 117a/ 117b | 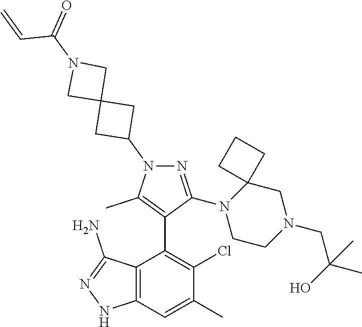 [SMILES: C=CC(=O)N1CC2(CC(n3nc(N4CCN(CC(C)(C)O)CC45CCC5)c(-c4c(Cl)c(C)cc5[nH]nc(N)c45)c3C)C2)C1] | Using Method-4 from Intermediate C29 (Step 2) and Intermediate D11 (Step 3) and C- SFC-1 (mobile phase: CO₂/[IPA + 0.1% Et₃N] 65/35); Example 117a = 1^{st} eluting isomer, Example 117b = 2^{nd} eluting isomer | Example 117a: UPLC-MS-4: Rt = 0.69 min; MS m/z [M + H]⁺ 607.6/609.6; C-SFC-3 (mobile phase: CO₂/[IPA + 0.1% NH₃] 65/35): Rt = 2.36 min, Example 117b: C-SFC-3 (mobile phase: CO₂/[IPA + 0.1% NH₃] 65/35): Rt = 3.53 min. |
| | 1-(6-(4-(3-amino-5-chloro- | | |
| | 6-methyl-1H-indazol-4-yl)- | | |
| | 3-(8-(2-hydroxy-2- | | |
| | methylpropyl)-5,8- | | |
| | diazaspiro[3.5]nonan-5-yl)- | | |
| | 5-methyl-1H-pyrazol-1-yl)- | | |
| | 2-azaspiro[3.3]heptan-2- | | |
| | yl)prop-2-en-1-one | | |
| 118a/ 118b | 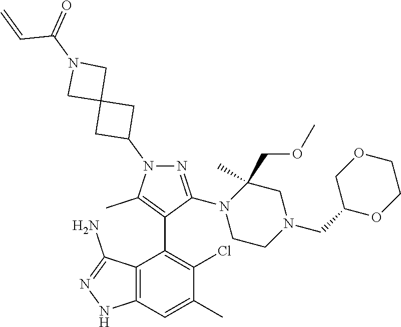 [SMILES: C=CC(=O)N1CC2(CC(n3nc(N4CCN(C[C@@H]5COCCO5)C[C@]4(C)COC)c(-c4c(Cl)c(C)cc5[nH]nc(N)c45)c3C)C2)C1] | Using Method-4 from Intermediate C66 and Intermediate D11 (Step 3) and C- SFC-5 (mobile phase: CO₂/[MeOH + 0.1% Et₃N] 70/30); Example 118a = 1^{st} eluting isomer, Example 118b = 2^{nd} eluting isomer | Example 118a: ¹H NMR (400 MHz, DMSO-d₆) δ 11.5 (s, 1H), 7.19 (s, 1H), 6.30 (m, 1H), 6.09 (m, 1H), 5.67 (m, 1H), 4.72 (m, 1H), 4.40 (s, 2H), 4.32 (s, 1H), 4.29 (m, 1H), 4.04 (s, 1H), 4.00 (m, 1H), 3.80 (m, 1H), 3.56- 3.72 (m, 3H), 3.44-3.55 (m, 3H), 3.38 (m, 1H), 3.15 (m, 1H), 3.14 (s, 3H), 2.83 (m, 2H), 2.73 (m, 3H), 2.61 (m, 1H), 2.41 (s, 3H), 2.27 (m, 1H), 2.05-2.20 (m, 4H), 1.90 (m, 1H), 1.88 (s, 3H), 1.19 (s, 3H); UPLC-MS- 2e: Rt = 3.04 min; MS m/z [M + H]⁺ 653.5/655.5; C-SFC-6 (mobile phase: CO₂/[MeOH + 0.1% NH₃] 70/30): Rt = 2.71 min, Example 118b: C-SFC-6 (mobile phase: CO₂/[MeOH + 0.1% NH₃] 70/30): Rt = 3.40 min. |
| | 1-(6-(3-((R)-4-(((R)-1,4- | | |
| | dioxan-2-yl)methyl)-2- | | |
| | (methoxymethyl)-2- | | |
| | methylpiperazin-1-yl)-4-(3- | | |
| | amino-5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 119a/ 119b | 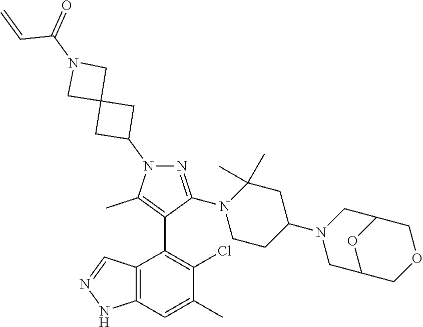 [SMILES: C=CC(=O)N1CC2(CC(n3nc(N4CCC(N5CC6COCC(C5)O6)CC4(C)C)c(-c4c(Cl)c(C)cc5[nH]ncc45)c3C)C2)C1] | Using Method-4 from Intermediate C98b (Step 3) and C-HPLC-35 (mobile phase: [Heptane + 0.05% Et₃N]/[EtOH + 0.05% Et₃N] 20/80); Example 119a = 1^{st} eluting isomer, Example 119b = 2^{nd} eluting isomer | Example 119b: UPLC-MS-2e: Rt = 3.39 min; MS m/z [M + H]⁺ 634.5/636.5; C-HPLC-22 (mobile phase: [Heptane + 0.05% DEA]/[EtOH + 0.05% DEA] 20/80): Rt = 10.06 min, Example 119a: C-HPLC-22 (mobile phase: [Heptane + 0.05%DEA]/[EtOH + 0.05% DEA] 20/80): Rt = 4.90 min. |
| | 1-(6-(3-(4-(3,9-dioxa-7- | | |
| | azabicyclo[3.3.1]nonan-7- | | |
| | yl)-2,2-dimethylpiperidin-1- | | |
| | yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2- | | |
| | yl)prop-2-en-1-one | | |
| 120a/ 120b | 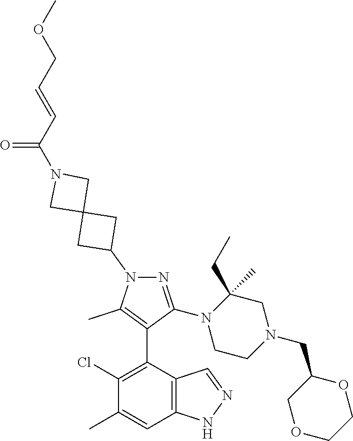 [SMILES: CC[C@@]1(C)CN(C[C@@H]2COCCO2)CCN1c1nn(C2CC3(C2)CN(C(=O)/C=C/COC)C3)c(C)c1-c1c(Cl)c(C)cc2[nH]ncc12] | Using Method-4d from Intermediate C65 (Step 3) and (E)-4- methoxybut-2- enoic acid (CAS [75933-65-2]) (Step 5) and C-SFC-4 (mobile phase: CO₂/[IPA + 0.1% Et₃N] 70/30); Example 120a = 1^{st} eluting atropisomer Example 120b = 2^{nd} eluting atropisomer | Example 120b: UPLC-MS-4: Rt = 0.68 min; MS m/z [M + H]⁺ 666.5/668.5; C-SFC-3 (mobile phase: CO₂/[IPA + 0.1% NH₃] 70/30): Rt = 2.46 min, Example 120a: C-SFC-3 (mobile phase: CO₂/[IPA + 0.1% NH₃] 70/30): Rt = 1.76 min. |
| | (E)-1-(6-(3-((S)-4-(((R)- | | |
| | 1,4-dioxan-2-yl)methyl)-2- | | |
| | ethyl-2-methylpiperazin-1- | | |
| | yl)-4-(5-chloro-6-methyl- | | |
| | 1H-indazol-4-yl)-5-methyl- | | |
| | 1H-pyrazol-1-yl)-2- | | |
| | azaspiro[3.3]heptan-2-yl)- | | |
| | 4-methoxybut-2-en-1-one | | |

### Method-5 for the Preparation of Example 121a and 121b: (S)-1-(6-(3-(4-Acetyl-2-ethyl-2-methylpiperazin-1-yl)-4-(5-chloro-6-methyl-1H-indazol-4-yl)-5-methyl-1H-pyrazol-1-yl)-2-azaspiro[3.3]heptan-2-yl)prop-2-en-1-one

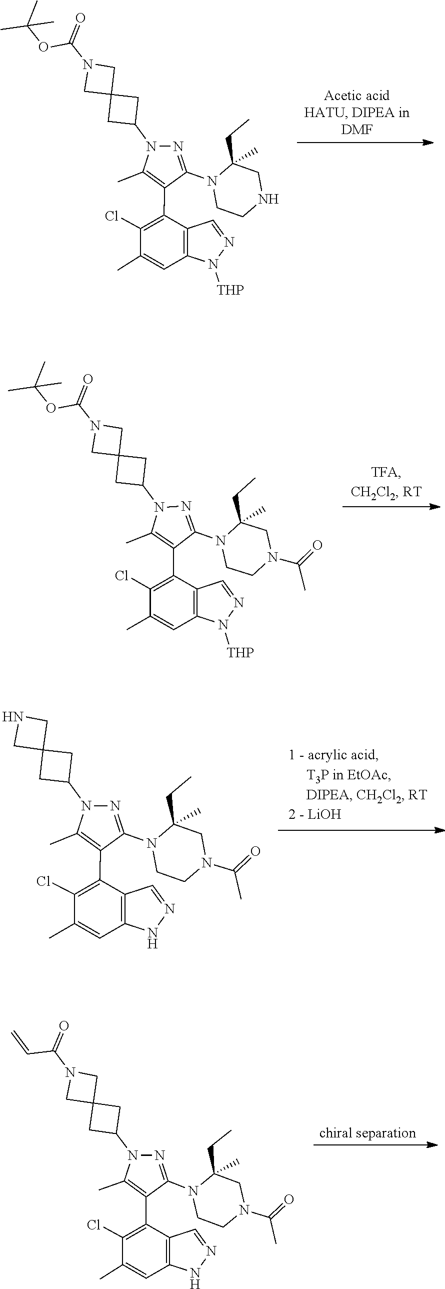

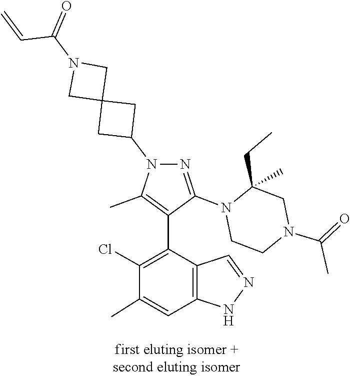 [SMILES: C=CC(=O)N1CC2(CC(n3nc(N4CCN(C(C)=O)C[C@]4(C)CC)c(-c4c(Cl)c(C)cc5[nH]ncc45)c3C)C2)C1]

### Step 1: Tert-butyl 6-(3-((S)-4-acetyl-2-ethyl-2-methylpiperazin-1-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate

To a solution of acetic acid (0.035 mL, 0.61 mmol) in DMF (4 mL) was added at room temperature DIPEA (0.22 mL, 1.23 mmol) followed by a solution of HATU (466 mg, 1.23 mmol) in DMF (4 mL). After 5 min at RT, a solution of (S)-1-(6-(4-(5-chloro-6-methyl-1H-indazol-4-yl)-3-(2-ethyl-2-methylpiperazin-1-yl)-5-methyl-1H-pyrazol-1-yl)-2-azaspiro[3.3]heptan-2-yl)prop-2-en-1-one (Intermediate C39, 400 mg, 0.61 mmol) in DMF (4 mL) was added and the reaction mixture was stirred at RT for 30 min. CH₂Cl₂ (30 mL) and K₂CO₃ (10% in water, 10 mL) were added, the layers were separated and the aqueous layer was extracted with CH₂Cl₂. The combined organic extracts were washed with brine, dried (MgSO₄), filtered and evaporated. The crude residue was purified by normal phase chromatography (eluent: EtOAc in heptane 0 to 50%). UPLC-MS-2a: Rt=1.27 min; MS m/z [M+H]⁺ 694.5/696.7.

### Step 2: (S)-1-(4-(4-(5-chloro-6-methyl-1H-indazol-4-yl)-5-methyl-1-(2-azaspiro[3.3]heptan-6-yl)-1H-pyrazol-3-yl)-3-ethyl-3-methylpiperazin-1-yl)ethan-1-one

To a solution of tert-butyl 6-(3-((S)-4-acetyl-2-ethyl-2-methylpiperazin-1-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Step 1, 420 mg, 0.61 mmol) in CH₂Cl₂ (2 mL) was added TFA (1.40 mL, 18.1 mmol). The reaction mixture was stirred at RT for 2 h, then was evaporated to dryness to give the title compound as a trifluoroacetate salt which was used in the next step without purification. UPLC-MS-2a: Rt=0.79 min; MS m/z [M+H]⁺ 510.3/512.3.

### Step 3: (S)-1-(6-(3-(4-acetyl-2-ethyl-2-methylpiperazin-1-yl)-4-(5-chloro-6-methyl-1H-indazol-4-yl)-5-methyl-1H-pyrazol-1-yl)-2-azaspiro[3.3]heptan-2-yl)prop-2-en-1-one

To a stirred solution of (S)-1-(4-(4-(5-chloro-6-methyl-1H-indazol-4-yl)-5-methyl-1-(2-azaspiro[3.3]heptan-6-yl)-1H-pyrazol-3-yl)-3-ethyl-3-methylpiperazin-1-yl)ethan-1-one trifluoroacetate (Step 2, 0.59 mmol) in CH₂Cl₂ (3 mL) was added at 0° C. under Argon atmosphere a solution of acrylic acid (0.12 mL, 1.76 mmol), T₃P (50% in EtOAc, 1.05 mL, 1.76 mmol) and DIPEA (2.56 mL, 14.65 mmol) in CH₂Cl₂ (3 mL). The reaction mixture was stirred for 30 min at 0° C. Then, the RM was quenched by addition of a sat. aq. solution of NaHCO₃ (50 mL) and extracted with CH₂Cl₂ (2×100 mL). The combined organic extracts were washed with a sat. aq. solution of NaHCO₃, brine, dried (MgSO₄), filtered and evaporated. The crude residue was diluted in THF (20 mL) and LiOH (2 M, 2.93 mL, 5.86 mmol) was added. The mixture was stirred at RT for 30 min, then extracted with CH₂Cl₂, washed with brine, dried (MgSO₄), filtered and evaporated. The crude residue was purified by normal phase chromatography (eluent: MeOH/CH₂Cl₂ 10/90 in CH₂Cl₂ from 0 to 100%) to give the title compound. The isomers were separated by chiral SFC (C-SFC-1; mobile phase: CO₂/[IPA+0.1% Et₃N] 65/35) to give the title compound Example 121b as the second eluting peak: ¹H NMR (600 MHz, DMSO-d₆) δ 13.0 (br. s, 1H), 7.59 (m, 1H), 7.47 (s, 1H), 6.30 (m, 1H), 6.09 (m, 1H), 5.67 (m, 1H), 4.75 (m, 1H), 4.33 (s, 1H), 4.28 (s, 1H), 4.05 (s, 1H), 3.99 (s, 1H), 3.47-3.15 (m, 3H, overlapping with DMSO peak), 3.08 (m, 1H), 2.93-2.81 (m, 2H), 2.76-2.63 (m, 4H), 2.48 (s, 3H), 1.98 (s, 3H), 1.94 (s, 1.5H), 1.91 (s, 1.5H), 1.70-1.56 (m, 1H), 1.49-1.34 (m, 1H), 0.83-080 (m, 3H), 0.60-0.52 (m, 3H); UPLC-MS-2a: Rt=0.97 min; MS m/z [M+H]⁺ 564.3/566.3; C-SFC-3 (mobile phase: CO₂/[EtOH+0.1% Et₃N]: 65/35): Rt=2.51 min. The other isomer Example 121a was obtained as the first eluting peak: C-SFC-3 (mobile phase: CO₂/[EtOH+0.1% Et₃N]: 65/35): Rt=1.00 min.

Method-5a: similar to Method-5 except that Step d was performed using RCOCl and Et₃N in CH₂Cl₂ instead of RCOOH, HATU, DIPEA in DMF.

Method-5b: similar to Method-5 except Step 3 was performed using acryloyl chloride and NaHCO₃ followed by a treatment with LiOH as described in Method-8 Step 3.

Method-5c: similar to Method-5 except that Step 1 was performed using RCOOH, T₃P and DIPEA in CH₂Cl₂ as described in Step 3 instead of RCOOH, HATU, DIPEA in DMF.

The following examples 122a to 129b were prepared using analogous methods to Method-5 from intermediates (in Step 1) described in the intermediates synthesis section or commercially available. When the final separation of isomers is performed by preparative HPLC using acidic conditions the purified fractions were extracted with a sat. aq. NaHCO₃ solution before lyophilization from a mixture of CH₃CN/H₂O to give the title compound as a free base.

| | | Method, | |
|---|---|---|---|
| | | intermediates (Step | |
| | | 1) and chiral | |
| | | separation | |
| | | conditions used and | |
| Example | Structure | order of elution | Characterizing data |
| 122a/ 122b | 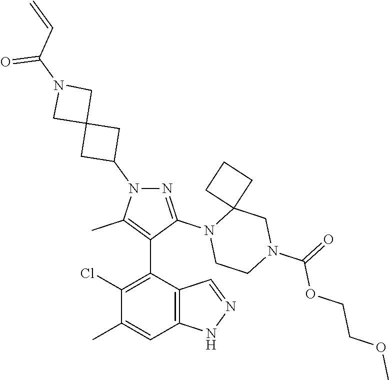 [SMILES: C=CC(=O)N1CC2(CC(n3nc(N4CCN(C(=O)OCCOC)CC45CCC5)c(-c4c(Cl)c(C)cc5[nH]ncc45)c3C)C2)C1] | Using Method-5a from Intermediate C28 and CAS: [628- 12-6] and C-SFC-1 (mobile phase: 35:65 [IPA + 0.1% Et₃N]/CO₂); Example 122a = 1^{st} eluting isomer, Example 122b = 2^{nd} eluting isomer | Example 122b: ¹H NMR (600 MHz, DMSO-d₆) δ 13.00 (s, 1H), 7.60 (s, 1H), 7.46 (s, 1H), 6.30 (m, 1H), 6.10 (m, 1H), 5.66 (m, 1H), 4.74 (m, 1H), 4.33 (s, 1H), 4.28 (s, 1H), 4.08 (m, 2H), 4.05 (s, 1H), 3.99 (s, 1H), 3.54 (m, 1H), 3.47 (m, 2H), 3.25 (m, 1H), 3.23 (s, 3H), 2.96 (m, 1H), 2.62-2.79 (m, 6H), 2.48 (s, 3H), 2.10 (m, 1H), 1.98 (s, 3H), 1.94 (m, 1H), 1.53-1.73 (m, 5H). UPLC-MS-4: Rt = 1.04 min, MS m/z [M + H]⁺ 622.5/624.6; C-SFC-3 (mobile phase: 35:65 [IPA + 0.025% NH₃]/CO₂): Rt = 2.69 min. Example 122a: C-SFC-3 (mobile phase: 35:65 [IPA + 0.025% NH₃]/CO₂): Rt = 1.32 min. |
| | 2-methoxyethyl 5-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-5,8- | | |
| | diazaspiro[3.5]nonane-8- | | |
| | carboxylate | | |
| 123a/ 123b | 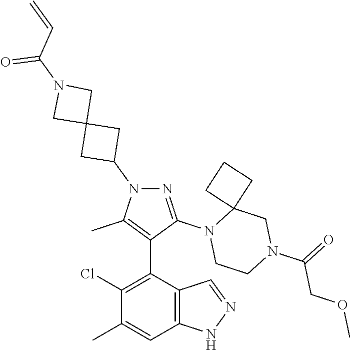 [SMILES: C=CC(=O)N1CC2(CC(n3nc(N4CCN(C(=O)COC)CC45CCC5)c(-c4c(Cl)c(C)cc5[nH]ncc45)c3C)C2)C1] 1-(6-(4-(5-chloro-6-methyl- 1H-indazol-4-yl)-3-(8-(2- | Using Method-5a from Intermediate C28 and CAS: [38870-89-2] and C- SFC-1 (mobile phase: 35:65 [IPA + 0.1% Et₃N]/CO₂); Example 123a = 1^{st} eluting isomer, Example 123b = 2^{nd} eluting isomer | Example 123b: ¹H NMR (600 MHz, DMSO-d₆) δ 13.00 (s, 1H), 7.61 (s, 1H), 7.46 (s, 1H), 6.30 (m, 1H), 6.09 (m, 1H), 5.67 (m, 1H), 4.75 (m, 1H), 4.34 (s, 1H), 4.27 (s, 1H), 4.05 (s, 1H), 4.04 (m, 1H), 4.00 (m, 1H), 3.99 (s, 1H), 3.76 (m, 0.5H), 3.46 (m, 0.5H), 3.26 (s, 1.5H), 3.22 (s, 1.5H), 3.30- 3.13 (m, 2H), 2.86-3.05 (m, 1H), 2.61-2.76 (m, 6H), 2.48 (s, 3H), 2.10 (m, 1H), 1.99 (s, 3H), 1.92 (m, 1H), 1.54-1.77 (m, 4H). UPLC-MS-2a: Rt = 0.97 min, MS m/z [M + H]⁺ 592.3/594.3; C-SFC-3 (mobile phase: 35:65 [IPA + 0.1% Et₃N]/CO₂): Rt = 2.44 min. Example 123a: C- SFC-3 (mobile phase: 35:65 [IPA + 0.1% Et₃N]/CO₂): Rt = 1.37 min. |
| | methoxyacetyl)-5,8- | | |
| | diazaspiro[3.5]nonan-5-yl)- | | |
| | 5-methyl-1H-pyrazol-1-yl)- | | |
| | 2-azaspiro [3.3]heptan-2- | | |
| | yl)prop-2-en-1-one | | |
| 124a/ 124b | 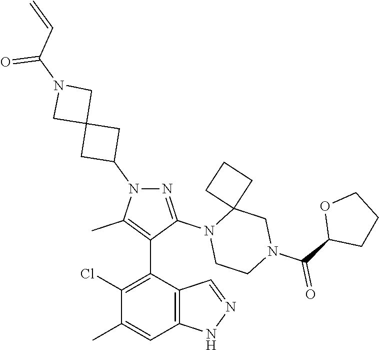 [SMILES: C=CC(=O)N1CC2(CC(n3nc(N4CCN(C(=O)[C@@H]5CCCO5)CC45CCC5)c(-c4c(Cl)c(C)cc5[nH]ncc45)c3C)C2)C1] (S)-1-(6-(4-(5-chloro-6- | Using Method-5 from Intermediate C28 and (S)-tetrahydrofuran-2- carboxylic acid and C- SFC-1 (mobile phase: CO₂/IPA 65/35); Example 124a = 1^{st} eluting isomer, Example 124b = 2^{nd} eluting isomer | Example 124b: ¹H NMR (400 MHz, DMSO-d₆) δ 13.00 (s, 1H), 7.62 (s, 1H), 7.46 (s, 1H), 6.30 (m, 1H), 6.10 (m, 1H), 5.67 (m, 1H), 4.75 (m, 1H), 4.60 (q, 1H), 4.34 (s, 1H), 4.27 (s, 1H), 4.05 (s, 1H), 3.99 (s, 1H), 3.80-3.66 (m, 3H), 3.50 (m, 0.5H), 3.38 (m, 0.5H), 3.25 (m, 0.5H), 3.11 (m, 0.5H), 2.87 (m, 0.5H), 2.78-2.61 (m, 6.5H), 2.49 (s, 3H), 2.09 (m, 1H), 1.99 (s, 3H), 1.97-1.88 (m, 3H), 1.86-1.71 (m, 2H), 1.65-1.55 (m, 4H). UPLC- MS-2a: Rt = 0.99 min, MS m/z [M + H]⁺ 618.3/620.3; C- SFC-3 (mobile phase: CO₂/IPA 65/35): Rt = 3.08 min. Example 124a: C-SFC- 3 (mobile phase: CO₂/IPA 65/35): Rt = 2.00 min. |
| | methyl-1H-indazol-4-yl)-5- | | |
| | methyl-3-(8- | | |
| | (tetrahydrofuran-2- | | |
| | carbonyl)-5,8- | | |
| | diazaspiro[3.5]nonan-5-yl)- | | |
| | 1H-pyrazol-1-yl)-2- | | |
| | azaspiro[3.3]heptan-2- | | |
| | yl)prop-2-en-1-one | | |
| 125a/ 125b | 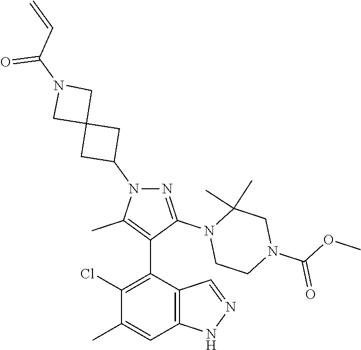 [SMILES: C=CC(=O)N1CC2(CC(n3nc(N4CCN(C(=O)OC)CC4(C)C)c(-c4c(Cl)c(C)cc5[nH]ncc45)c3C)C2)C1] | Using Method-5a from Intermediate C38 and chloroformate and C- SFC-4 (mobile phase: CO₂/EtOH 70/30); Example 125a = 1^{st} eluting isomer, Example 125b = 2^{nd} eluting isomer | Example 125b: ¹H NMR (400 MHz, DMSO-d₆) δ 13.00 (s, 1H), 7.59 (s, 1H), 7.48 (s, 1H), 6.31 (m, 1H), 6.10 (m, 1H), 5.67 (m, 1H), 4.75 (m, 1H), 4.34 (s, 1H), 4.29 (s, 1H), 4.05 (s, 1H), 3.99 (s, 1H), 3.55 (s, 3H), 3.13-3.04 (m, 2H), 2.98-2.85 (m, 4H), 2.76-2.66 (m, 4H), 2.48 (s, 3H), 1.99 (s, 3H), 1.00 (s, 1.5H), 0.99 (s, 1.5H), 0.94 (s, 1.5H), 0.92 (s, 1.5H). UPLC-MS-2a: Rt = 1.01 min, MS m/z [M + H]⁺ 566.3/ 568.3; C-SFC-3 (mobile phase: CO₂/EtOH 70/30): Rt = 2.85 min. Example 125a: C-SFC-3 (mobile phase: CO₂/EtOH 70/30): Rt = 1.14 min. |
| | methyl 4-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-3,3- | | |
| | dimethylpiperazine-1- | | |
| | carboxylate | | |
| 126a/ 126b | 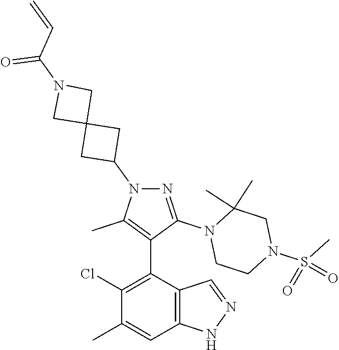 [SMILES: C=CC(=O)N1CC2(CC(n3nc(N4CCN(S(C)(=O)=O)CC4(C)C)c(-c4c(Cl)c(C)cc5[nH]ncc45)c3C)C2)C1] | Using Method-5ab from Intermediate C38 and mesyl chloride and C-SFC-2 (mobile phase: CO₂/iPrOH 70/30); Example 126a = 1^{st} eluting isomer, Example 126b = 2^{nd} eluting isomer | Example 126a: ¹H NMR (600 MHz, DMSO-d₆) δ 13.00 (s, 1H), 7.59 (s, 1H), 7.48 (s, 1H), 6.31 (m, 1H), 6.11 (m, 1H), 5.68 (m, 1H), 4.75 (m, 1H), 4.35 (s, 1H), 4.30 (s, 1H), 4.06 (s, 1H), 4.01 (s, 1H), 3.09-2.99 (m, 2H), 2.86-2.81 (m, 2H), 2.77 (s, 1.5H), 2.76 (s, 1.5H), 2.75-2.60 (m, 5H), 2.64-2.61 (m, 1H), 2.49 (s, 3H), 1.99 (s, 3H), 1.06 (s, 1.5H), 1.05 (s, 1.5H), 1.02 (s, 1.5H), 1.01 (s, 1.5H). UPLC-MS-2a: Rt = 0.94 min, MS m/z [M + H]⁺ 586.3/588.3; C-SFC-3 (mobile phase: CO₂/iPrOH 70/30): Rt = 2.28 min. Example 126b: C-SFC-3 (mobile phase: CO₂/iPrOH 70/30): Rt = 1.52 min. |
| | 1-(6-(4-(5-chloro-6-methyl- | | |
| | 1H-indazol-4-yl)-3-(2,2- | | |
| | dimethyl-4- | | |
| | (methylsulfonyl)piperazin- | | |
| | 1-yl)-5-methyl-1H-pyrazol- | | |
| | 1-yl)-2- | | |
| | azaspiro[3.3]heptan-2- | | |
| | yl)prop-2-en-1-one | | |
| 127a/ 127b | 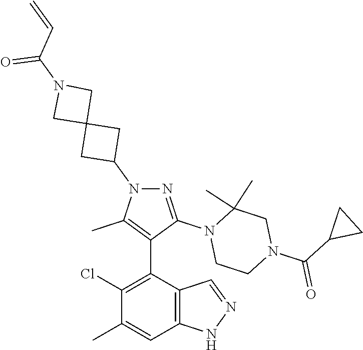 [SMILES: C=CC(=O)N1CC2(CC(n3nc(N4CCN(C(=O)C5CC5)CC4(C)C)c(-c4c(Cl)c(C)cc5[nH]ncc45)c3C)C2)C1] | Using Method-5ab from Intermediate C38 and cyclopropanecarbonyl chloride and C-SFC-2 (mobile phase: CO₂/iPrOH 55/45); Example 127a = 1^{st} eluting isomer, Example 127b = 2^{nd} eluting isomer | Example 127b: ¹H NMR (600 MHz, DMSO-d₆) δ 13.02 (s, 1H), 7.61 (s, 1H), 7.47 (s, 1H), 6.31 (m, 1H), 6.10 (m, 1H), 5.69 (m, 1H), 4.76 (m, 1H), 4.34 (s, 1H), 4.29 (s, 1H), 4.07 (s, 1H), 4.00 (s, 1H), 3.34-3.12 (m, 3H), 3.10-2.80 (m, 3H), 2.77- 2.67 (m, 4H), 2.49 (s, 3H), 1.99 (s, 3H), 1.90 (m, 1H), 1.04-0.93 (m, 6H), 0.72-0.62 (m, 4H); UPLC-MS-2e: Rt = 4.74 min, MS m/z [M + H]⁺ 576.3/578.3; C-SFC-3 (mobile phase: CO₂/iPrOH 55/45): Rt = 2.48 min. Example 127a: C-SFC-3 (mobile phase: CO₂/iPrOH 55/45): Rt = 0.81 min. |
| | 1-(6-(4-(5-chloro-6-methyl- | | |
| | 1H-indazol-4-yl)-3-(4- | | |
| | (cyclopropanecarbonyl)- | | |
| | 2,2-dimethylpiperazin-1- | | |
| | yl)-5-methyl-1H-pyrazol-1- | | |
| | yl)-2-azaspiro[3.3]heptan- | | |
| | 2-yl)prop-2-en-1-one | | |
| 128a/ 128b | 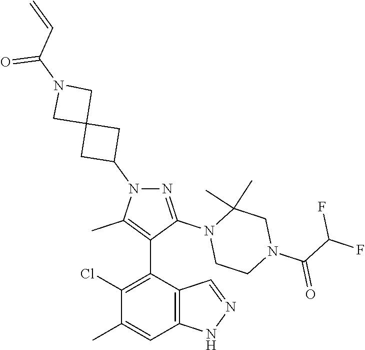 [SMILES: C=CC(=O)N1CC2(CC(n3nc(N4CCN(C(=O)C(F)F)CC4(C)C)c(-c4c(Cl)c(C)cc5[nH]ncc45)c3C)C2)C1] | Using Method-5bc from Intermediate C38 and 2,2- difluoroacetic acid and C-SFC-26 (mobile phase: CO₂/iPrOH 60/40); Example 128a = 1^{st} eluting isomer, Example 128b = 2^{nd} eluting isomer | Example 128b: ¹H NMR (600 MHz, DMSO-d₆) δ 13.04 (s, 1H), 7.62 (s, 1H), 7.49 (s, 1H), 6.66 (qq, 1H), 6.31 (m, 1H), 6.10 (m, 1H), 5.69 (m, 1H), 4.77 (m, 1H), 4.34 (s, 1H), 4.28 (s, 1H), 4.06 (s, 1H), 4.00 (m, 1H), 3.26 (−3.17 (m, 2H), 3.12 (t, 1H), 3.05-2.96 (m, 3H), 2.77- 2.68 (m, 4H), 2.49 (s, 3H), 2.01 (s, 3H), 1.01-0.93 (m, 6H). UPLC-MS-2e: Rt = 4.69 min, MS m/z [M + H]⁺ 586.3/ 588.3; C-SFC-8 (mobile phase: CO₂/iPrOH 60/40): Rt = 2.24 min. Example 128a: C-SFC-3 (mobile phase: CO₂/iPrOH 60/40): Rt = 1.51 min. |
| | 1-(6-(4-(5-chloro-6-methyl- | | |
| | 1H-indazol-4-yl)-3-(4-(2,2- | | |
| | difluoroacetyl)-2,2- | | |
| | dimethylpiperazin-1-yl)-5- | | |
| | methyl-1H-pyrazol-1-yl)-2- | | |
| | azaspiro[3.3]heptan-2- | | |
| | yl)prop-2-en-1-one | | |
| 129a/ 129b | 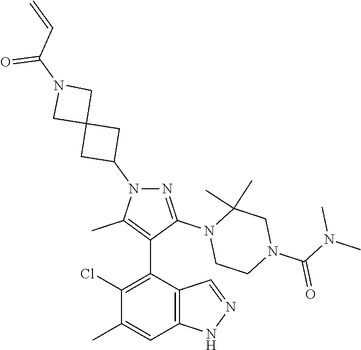 [SMILES: C=CC(=O)N1CC2(CC(n3nc(N4CCN(C(=O)N(C)C)CC4(C)C)c(-c4c(Cl)c(C)cc5[nH]ncc45)c3C)C2)C1] | Using Method-5ab from C38 and dimethylcarbamic chloride and C-SFC- 26 (mobile phase: CO₂/[iPrOH + 0.1% Et₃N]57/43); Example 129a = 1^{st} eluting isomer, Example 129b = 2^{nd} eluting isomer | Example 129a: ¹H NMR (600 MHz, DMSO-d₆) δ 13.01 (s, 1H), 7.59 (s, 1H), 7.47 (s, 1H), 6.30 (m, 1H), 6.10 (m, 1H), 5.67 (m, 1H), 4.76 (m, 1H), 4.34 (s, 1H), 4.29 (s, 1H), 4.06 (s, 1H), 4.00 (s, 1H), 2.96-2.87 (m, 2H), 2.82-22.76 (m, 1H), 2.76-2.63 (m, 13H), 2.49 (s, 3H), 2.00 (s, 3H), 1.08 (s, 1.5H), 1.07 (s, 1.5H), 1.00 (s, 1.5H), 0.99(s, 1.5H). UPLC- MS-2e: Rt = 4.67 min, MS m/z [M + H]⁺ 579.4/581 .; C- SFC-8 (mobile phase: CO₂/ [iPrOH + 0.1% Et₃N] 55/45): Rt = 2.40 min. Example 129b: C-SFC-8 (mobile phase: CO₂/[iPrOH + 0.1% Et₃N] 55/45): Rt = 2.43 min. |
| | 4-(1-(2-acryloyl-2- | | |
| | azaspiro[3.3]heptan-6-yl)- | | |
| | 4-(5-chloro-6-methyl-1H- | | |
| | indazol-4-yl)-5-methyl-1H- | | |
| | pyrazol-3-yl)-N,N,3,3- | | |
| | tetramethylpiperazine-1- | | |
| | carboxamide | | |

### Method-6 for the Preparation of Example 130a and 130b: 1-(6-(4-(5-Chloro-6-methyl-1H-indazol-4-yl)-5-methyl-3-(8-(tetrahydro-2H-pyran-4-yl)-5,8-diazaspiro[3.5]nonan-5-yl)-1H-pyrazol-1-yl)-2-azaspiro[3.3]heptan-2-yl)prop-2-en-1-one

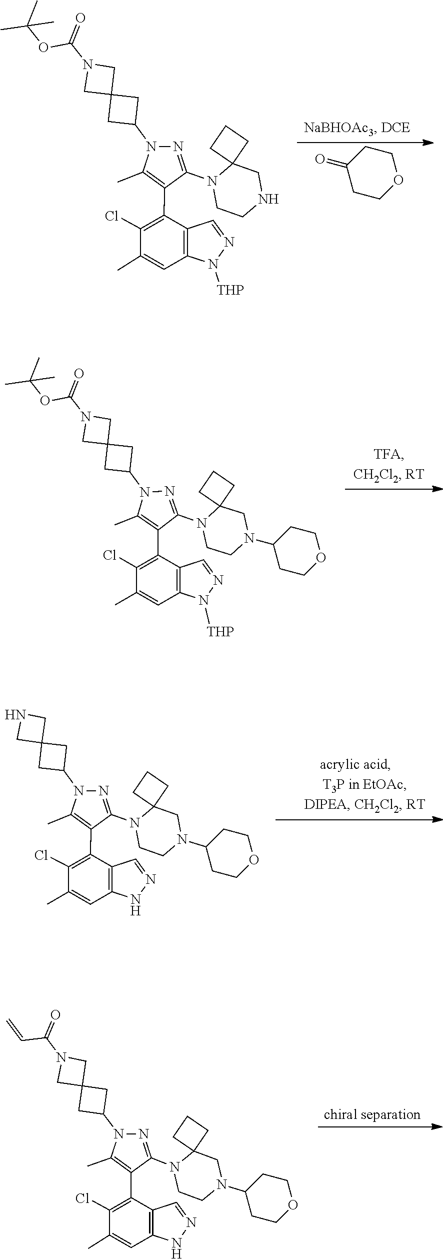

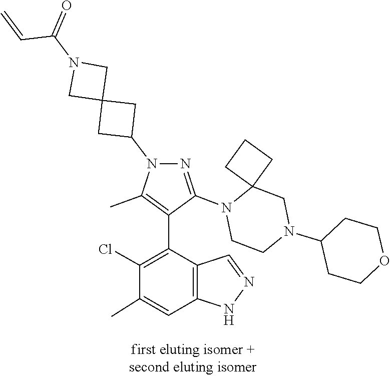 [SMILES: C=CC(=O)N1CC2(CC(n3nc(N4CCN(C5CCOCC5)CC45CCC5)c(-c4c(Cl)c(C)cc5[nH]ncc45)c3C)C2)C1]

### Step 1: Tert-butyl 6-(4-(5-chloro-6-methyl-1-(tetrahydro-2H-pyran-2-yl)-1H-indazol-4-yl)-5-methyl-3-(8-(tetrahydro-2H-pyran-4-yl)-5,8-diazaspiro[3.5]nonan-5-yl)-1H-pyrazol-1-yl)-2-azaspiro[3.3]heptane-2-carboxylate

To a stirred solution of tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate (intermediate C28, 400 mg, 0.62 mmol) in dichloroethane (3 mL) were added under argon atmosphere tetrahydro-4H-pyran-4-one (123 mg, 1.23 mmol) and NaBH(OAc)₃ (391 mg, 1.85 mmol). The reaction mixture was stirred for 16 h at RT. The reaction mixture was quenched by addition of a sat. aq. NaHCO₃ solution and extracted CH₂Cl₂ (2×). The combined organic layers were washed with a sat. aq. NaHCO₃ solution, dried (Na₂SO₄) and evaporated. The crude residue was purified by normal phase chromatography (eluent: MeOH in CH₂Cl₂ from 0 to 10%). UPLC-MS-2a: Rt=1.06 min; MS m/z [M+H]⁺ 734.7/736.5.

### Step 2: 5-(4-(5-Chloro-6-methyl-1H-indazol-4-yl)-5-methyl-1-(2-azaspiro[3.3]heptan-6-yl)-1H-pyrazol-3-yl)-8-(tetrahydro-2H-pyran-4-yl)-5,8-diazaspiro[3.5]nonane

To a stirred solution of tert-butyl 6-(4-(5-chloro-6-methyl-1-(tetrahydro-2H-pyran-2-yl)-1H-indazol-4-yl)-5-methyl-3-(8-(tetrahydro-2H-pyran-4-yl)-5,8-diazaspiro[3.5]nonan-5-yl)-1H-pyrazol-1-yl)-2-azaspiro[3.3]heptane-2-carboxylate (Step 1, 448 mg, 0.61 mmol) in CH₂Cl₂ (4 mL) was added TFA (1.41 mL, 18.3 mmol) and the reaction mixture was stirred for 16 h at RT. The RM was concentrated to give the title compound as a trifluoroacetate salt, which was used in the next step without purification. UPLC-MS-2a: Rt=0.64 min; MS m/z [M+H]⁺ 550.3/552.3.

### Step 3: 1-(6-(4-(5-Chloro-6-methyl-1H-indazol-4-yl)-5-methyl-3-(8-(tetrahydro-2H-pyran-4-yl)-5,8-diazaspiro[3.5]nonan-5-yl)-1H-pyrazol-1-yl)-2-azaspiro[3.3]heptan-2-yl)prop-2-en-1-one

To a stirred solution of 5-(4-(5-chloro-6-methyl-1H-indazol-4-yl)-5-methyl-1-(2-azaspiro[3.3]heptan-6-yl)-1H-pyrazol-3-yl)-8-(tetrahydro-2H-pyran-4-yl)-5,8-diazaspiro[3.5]nonane trifluoroacteate (Step 2, 0.61 mmol) in CH₂Cl₂ (4 mL) was added under argon atmosphere a solution of acrylic acid (0.13 mL, 1.83 mmol), T₃P (50% in EtOAc, 1.09 mL, 1.83 mmol) and DIEA (2.67 mL, 15.3 mmol) at 0° C. and the reaction mixture was stirred for 30 min at 0° C. The RM was quenched by addition of a sat. aq. NaHCO₃ solution, then extracted with CH₂Cl₂ (2×). The combined organic extracts were washed with a sat. aq. NaHCO₃ solution, dried (Na₂SO₄) and evaporated. The crude residue was purified by normal phase chromatography (eluent: MeOH in CH₂Cl₂ from 0 to 10%) to give the title compound. The isomers were separated by chiral SFC (C-SFC-1; mobile phase: CO₂/[IPA+0.1% Et₃N] 70/30) to give the title compound Example 130b as the second eluting peak (white powder): ¹H NMR (400 MHz, DMSO-d₆) δ 12.95 (s, 1H), 7.57 (s, 1H), 7.44 (s, 1H), 6.31 (m, 1H), 6.10 (m, 1H), 5.67 (m, 1H), 4.75 (m, 1H), 4.34 (s, 1H), 4.29 (s, 1H), 4.06 (s, 1H), 4.00 (s, 1H), 3.81 (m, 2H), 3.21 (t, 2H), 2.79-2.63 (m, 6H), 2.47 (s, 3H), 2.44 (m, 1H), 2.36 (m, 1H), 2.25 (m, 2H), 2.16 (m, 3H), 1.99 (s, 3H), 1.76 (m, 1H), 1.62-1.52 (m, 5H), 1.38-1.26 (m, 2H); UPLC-MS-2a: Rt=0.79 min; MS m/z [M+H]⁺ 604.3/606.3; C-SFC-3 (mobile phase: CO₂/[IPA+0.1% Et₃N] 70/30): Rt=2.70 min. The other isomer Example 130a was obtained as the first eluting peak: C-SFC-3 (mobile phase: CO₂/[IPA+0.1% Et₃N]70/30): Rt=1.65 min.

Method-6a: similar to Method-6 except that Step 3 was performed using acryloyl chloride and NaHCO₃ as described in Method-8 Step 3.

The following examples 131a to 159b were prepared using analogous methods to Method-6 from intermediates (in Step 1) described in the intermediates synthesis section or commercially available.

In Step 3, If observed the side product resulting from reaction of the acryloyl chloride with the indazole NH was hydrolyzed by treatment with LiOH as described in Method-5 or in Method-12.

When the final separation of isomers is performed by preparative HPLC using acidic conditions the purified fractions were extracted with a sat. aq. NaHCO₃ solution before lyophilization from a mixture of CH₃CN/H₂O to give the title compound as a free base.

| | | Method, | |
|---|---|---|---|
| | | intermediates (in | |
| | | Step 1) and chiral | |
| | | separation | |
| | | conditions used | |
| Example | Structure | and order of elution | Characterizing data |
| 131a/ 131b | 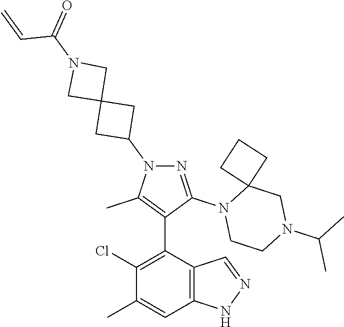 [SMILES: C=CC(=O)N1CC2(CC(n3nc(N4CCN(C(C)C)CC45CCC5)c(-c4c(Cl)c(C)cc5[nH]ncc45)c3C)C2)C1] 1-(6-(4-(5-chloro-6- methyl-1H-indazol-4-yl)- | Using Method-6 from Intermediate C28 and acetone (Step 1) and C-HPLC-1 (mobile phase: n- heptane/CH₂Cl₂/ EtOH 65/20/15); Example 131a = 1^{st} eluting isomer, Example 131b = 2^{nd} eluting isomer | Example 131b: ¹H NMR (400 MHz, DMSO-d₆) δ 12.9 (s, 1H), 7.57 (s, 1H), 7.43 (s, 1H), 6.31 (m, 1H), 6.10 (m, 1H), 5.67 (m, 1H), 4.74 (m, 1H), 4.34 (s, 1H), 4.28 (s, 1H), 4.05 (s, 1H), 4.00 (s, 1H), 2.80-2.63 (m, 6H), 2.50 (m, 1H), 2.47 (s, 3H), 2.39-2.30 (m, 2H), 2.25 (m, 1H), 2.17-2.07 (m, 3H), 1.98 (s, 3H), 1.77 (m, 1H), 1.64-1.53 (m, 3H), 0.89 (m, 6H). UPLC-MS-2a: Rt = 0.81 min; MS m/z [M + H]⁺ 562.4/ 564.4; C-HPLC-2 (mobile phase: n- heptane/CH₂Cl₂/EtOH 70/20/10): Rt = 7.28 min, Example 131a: C-HPLC-2 (mobile phase: n- heptane/CH₂Cl₂/EtOH 70/20/10): Rt = 5.17 min. |
| | 3-(8-isopropyl-5,8- | | |
| | diazaspiro[3.5]nonan-5- | | |
| | yl)-5-methyl-1H-pyrazol- | | |
| | 1-yl)-2- | | |
| | azaspiro[3.3]heptan-2- | | |
| | yl)prop-2-en-1-one | | |
| 132a/ 132b/ 132c/ 132d | 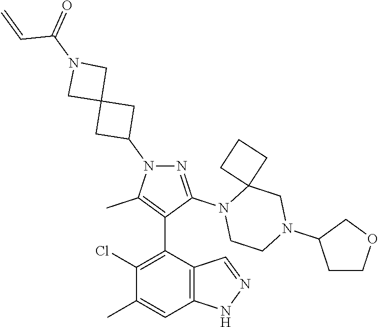 [SMILES: C=CC(=O)N1CC2(CC(n3nc(N4CCN(C5CCOC5)CC45CCC5)c(-c4c(Cl)c(C)cc5[nH]ncc45)c3C)C2)C1] 1-(6-(4-(5-chloro-6- methyl-1H-indazol-4-yl)-5-methyl-3-(8- (tetrahydrofuran-3-yl)-5,8- diazaspiro[3.5]nonan-5-yl)-1H-pyrazol-1-yl)-2- azaspiro[3.3]heptan-2-yl)prop-2-en-1-one | Using Method-6 from Intermediate C28 and dihydrofuran-3- one (Step 1) and C- SFC-4 (mobile phase: 30:70 [IPA + 0.1% Et₃N]/CO₂) to give Example 132c = 3^{rd} eluting isomer and Example 132d = 4^{th} eluting isomer and a mixture of the first and second eluting isomers Example 132a and Example 132b which were further separated by chiral HPLC C- HPLC-12 (mobile phase: 55:22.5:22.5 heptane/MTBE/EtO H + 0.1% Et₃N) to give Example 132a = 1^{st} eluting isomer, | Example 132c: ¹H NMR (600 MHz, DMSO-d₆) δ 12.95 (s, 1H), 7.57 (s, 1H), 7.43 (s, 1H), 6.30 (m, 1H), 6.10 (m, 1H), 5.67 (m, 1H), 4.75 (m, 1H), 4.34 (s, 1H), 4.28 (s, 1H), 4.05 (s, 1H), 4.00 (s, 1H), 3.86-4.03 (m, 1H), 3.65-3.78 (m, 2H), 3.58 (m, 1H), 3.40 (m, 1H), 3.10 (m, 1H), 2.62-2.84 (m, 7H), 2.47 (s, 3H), 2.23 (m, 2H), 2.10 (m, 2H), 1.98 (s, 3H), 1.88 (m, 1H), 1.76 (m, 1H), 1.49-1.66 (m, 4H). UPLC- MS-2a: Rt = 0.78 min, MS m/z [M + H]⁺ 590.5/592.5; C-SFC- 3 (mobile phase: 30:70 [IPA + 0.1% Et₃N]/CO₂): Rt = 2.25 min. Example 132d: ¹H NMR (400 MHz, DMSO-d₆) δ 12.95 (s, 1H), 7.56 (s, 1H), 7.43 (s, 1H), 6.30 (m, 1H), 6.09 (m, 1H), 5.67 (m, 1H), 4.75 (m, 1H), 4.34 (s, 1H), 4.28 (s, 1H), 4.05 (s, 1H), 4.00 (s, 1H), 3.64-3.78 (m, 2H), |
| | | Example 132b = 2^{nd} | 3.57 (m, 1H), 3.38 (m, 1H), |
| | | eluting isomer | 2.61-2.81 (m, 6H), 2.47 (m, |
| | | | 3H), 2.33 (m, 1H), 2.19-2.31 |
| | | | (m, 3H), 2.13 (m, 2H), 2.00 (m, |
| | | | 1H), 1.98 (s, 3H), 1.89 (m, |
| | | | 1H), 1.76 (m, 1H), 1.48-1.68 |
| | | | (m, 4H). UPLC-MS-X: Rt = |
| | | | 0.78 min, MS m/z [M + H]⁺ |
| | | | 590.5 − 592.5; C-SFC-3 |
| | | | (mobile phase: 30:70 |
| | | | [IPA + 0.1% Et₃N]/CO₂): Rt = |
| | | | 2.84 min. Example 132a: C- |
| | | | SFC-3 (mobile phase: 30:70 |
| | | | [IPA + 0.1% Et₃N]/CO₂): Rt = |
| | | | 1.75 min, Example 132b: C- |
| | | | SFC-3 (mobile phase: 30:70 |
| | | | [IPA + 0.1%Et₃N]/CO₂): Rt = |
| | | | 1.82 min. |
| 133a/ 133b | 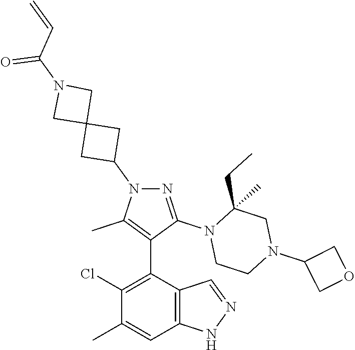 [SMILES: C=CC(=O)N1CC2(CC(n3nc(N4CCN(C5COC5)C[C@]4(C)CC)c(-c4c(Cl)c(C)cc5[nH]ncc45)c3C)C2)C1] (S)-1-(6-(4-(5-chloro-6- | Using Method-6 from Intermediate C39 and oxetanone (Step 1) and C-SFC-1 (mobile phase: CO₂/[IPA + 0.1% Et₃N] 74/26); Example 133a = 1^{st} eluting isomer, Example 133b = 2^{nd} eluting isomer | Example 133b: ¹H NMR (600 MHz, DMSO-d₆) δ 12.97 (s, 1H), 7.57 (m, 1H), 7.44 (s, 1H), 6.30 (m, 1H), 6.10 (m, 1H), 5.67 (m, 1H), 4.73 (m, 1H), 4.44 (m, 2H), 4.33 (s, 1H), 4.29 (s, 1H), 4.25-4.33 (m, 2H), 4.04 (s, 1H), 4.01 (s, 1H), 3.15 (m, 1H), 3.03 (m, 1H), 2.84 (m, 1H), 2.68-2.77 (m, 4H), 2.65 (m, 1H), 2.47 (s, 3H), 1.96 (s, 3H), 1.79-2.03 (m, 3H), 1.67 (m, 2H), 0.93 (m, 3H), 0.65 (m, 3H). UPLC-MS- 4: Rt = 0.79 min, MS m/z [M+ H]⁺ 578.5/580.5; C-SFC- 3 ( mobile phase: CO₂/[IPA + 0.025% NH₃] 74/26): Rt = 3.51 min. (600\ Example 133a : C-SFC-3 (mobile phase: CO₂/[IPA + 0.025% NH₃] 74/26): Rt = 2.23 min. |
| | methyl-1H-indazol-4-yl)- | | |
| | 3-(2-ethyl-2-methyl-4- | | |
| | (oxetan-3-yl)piperazin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 134a/ 134b | 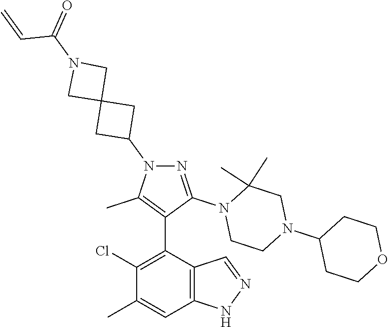 [SMILES: C=CC(=O)N1CC2(CC(n3nc(N4CCN(C5CCOCC5)CC4(C)C)c(-c4c(Cl)c(C)cc5[nH]ncc45)c3C)C2)C1] 1-(6-(4-(5-chloro-6- methyl-1H-indazol-4-yl)- | Using Method-6 from Intermediate C38 and tetrahydro-4H- pyran-4-one (Step 1) and C-SFC-1 (mobile phase: CO₂/[IPA + 0.1% Et₃N] 70/30); Example 134a = 1^{st} eluting isomer, Example 134b = 2^{nd} eluting isomer | Example 134b: ¹H NMR (400 MHz, DMSO-d₆) δ 12.9 (s, 1H), 7.57 (s, 1H), 7.44 (s, 1H), 6.30 (m, 1H), 6.09 (m, 1H), 5.67 (m, 1H), 4.72 (m, 1H), 4.33 (s, 1H), 4.27 (s, 1H), 4.05 (s, 1H), 4.00 (s, 1H), 3.79 (m, 2H), 3.18 (m, 2H), 2.80 (m, 2H), 2.74-2.68 (m, 4H), 2.47 (s, 3H), 2.19-2.12 (m, 2H), 2.07-2.00 (m, 2H), 1.97 (s, 3H), 1.57-1.50 (m, 2H), 1.30- 1.20 (m, 3H), 1.15 (m, 3H), 1.05 (m, 3H). UPLC-MS-2e: Rt = 3.12 min; MS m/z [M + H]⁺ 592.3/594.4; C-SFC-3 (mobile phase: CO₂/[IPA + 0.1% Et₃N] 70/30): Rt = 2.32 min, Example 134a: C-SFC-3 (mobile phase: CO₂/[IPA + 0.1% Et₃N] 70/30): Rt = 1.72 min. |
| | 3-(2,2-dimethyl-4- | | |
| | (tetrahydro-2H-pyran-4- | | |
| | yl)piperazin-1-yl)-5- | | |
| | methyl-1H-pyrazol-1-yl)- | | |
| | 2-azaspiro[3.3]heptan-2- | | |
| | yl)prop-2-en-1-one | | |
| 135a/ 135b | 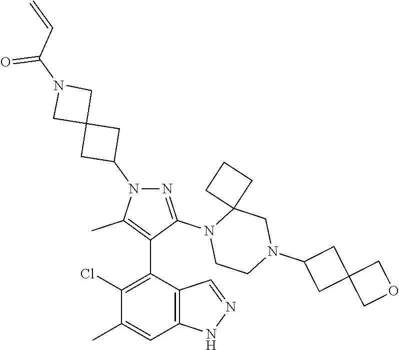 [SMILES: C=CC(=O)N1CC2(CC(n3nc(N4CCN(C5CC6(COC6)C5)CC45CCC5)c(-c4c(Cl)c(C)cc5[nH]ncc45)c3C)C2)C1] 1-(6-(3-(8-(2-oxaspiro[3.3]heptan-6- yl)-5,8-diazaspiro[3.5]nonan-5- | Using Method-6 from Intermediate C28 and 2- oxaspiro[3,3]heptan- 6-one (Step 1) and C-SFC-1 (mobile phase: CO₂/[IPA + 0.1% Et₃N] 72/28); Example 135a = 1^{st} eluting isomer, Example 135b = 2^{nd} eluting isomer | Example 135b: ¹H NMR (600 MHz, DMSO-d₆) δ 12.9 (s, 1H), 7.55 (s, 1H), 7.43 (s, 1H), 6.30 (m, 1H), 6.10 (m, 1H), 5.67 (m, 1H), 4.77-4.70 (m, 1H), 4.52 (m, 2H), 4.41 (m, 2H), 4.33 (s, 1H), 4.28 (m, 1H), 4.05 (s, 1H), 3.99 (m, 1H), 2.78-2.62 (m, 6H), 2.47 (s, 3H), 2.37-2.33 (m, 1H), 2.26-2.18 (m, 4H), 2.15-2.05 (m, 2H), 2.01-1.90 (m, 1H), 1.98 (s, 3H), 1.87-1.80 (m, 3H), 1.80-1.71 (m, 1H), 1.63- 1.51 (m, 3H). UPLC-MS-2e: Rt = 3.29 min, MS m/z [M + H]⁺ 616.3/618.3; C-SFC-3 (mobile phase: CO₂/[IPA + 0.025% NH₃] 72/28): Rt 2.82 min. Example 135a: C-SFC-3 (mobile phase: CO₂/[IPA + 0.025% NH₃] 72/28): Rt = 1.81 min. |
| | yl)-4-(5-chloro-6-methyl- | | |
| | 1H-indazol-4-yl)-5- | | |
| | methyl-1H-pyrazol-1-yl)- | | |
| | 2-azaspiro[3.3]heptan-2- | | |
| | yl)prop-2-en-1-one | | |
| 136a/ 136b | 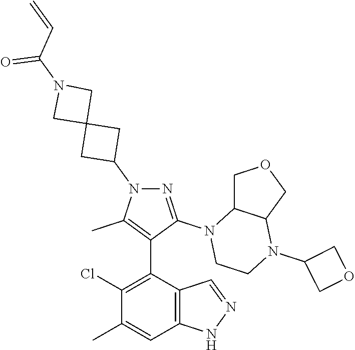 [SMILES: C=CC(=O)N1CC2(CC(n3nc(N4CCN(C5COC5)C5COCC54)c(-c4c(Cl)c(C)cc5[nH]ncc45)c3C)C2)C1] | Using Method-6 from Intermediate C36a and oxetanone (Step 1) and C-HPLC-15 (mobile phase: n- heptane/CH₂Cl₂/EtO H 60/25/15 + 0.1% Et₃N); Example 136a = 1^{st} eluting isomer, Example 136b = 2^{nd} eluting isomer | Example 136a: ¹H NMR (600 MHz, DMSO-d₆) δ 13.1 (s, 1H), 7.63 (s, 1H), 7.48 (s, 1H), 6.31 (m, 1H), 6.10 (m, 1H), 5.67 (m, 1H), 4.70 (m, 1H), 4.41 (t, 1H), 4.38-4.32 (m, 4H), 4.28 (m, 1H), 4.21 (t, 1H), 4.06 (m, 1H), 3.99 (m, 1H), 3.61 (t, 1H), 3.48-3.39 (m, 2H), 3.35-3.33 (m, 1H), 3.05 (m, 1H), 2.81-2.75 (m, 1H), 2.73-2.63 (m, 3H), 2.54-2.49 (m, 3H overlapping with DMSO peak), 2.48 (s, 3H), 2.14 (m, 1H), 1.90 (s, 3H), 1.69-1.64 (m, 1H). UPLC-MS- 2e: Rt = 3.66 min, MS m/z [M + H]⁺ 578.3/580.3; C- HPLC-16 (mobile phase: n- heptane/CH₂Cl₂/EtOH 60/25/1 + 0.05% Et₃N): Rt = 13.4 min. Example 136b: C- |
| | 1-(6-(4-(5-chloro-6- | | HPLC-16 (mobile phase: n- |
| | methyl-1H-indazol-4-yl)- | | heptane/CH₂Cl₂/EtOH |
| | 5-methyl-3-((4aS*,7aS*)- | | 60/25/15 + 0.05% Et₃N): Rt = |
| | 4-(oxetan-3- | | 19.4 min. |
| | yl)hexahydrofuro[3,4- | | |
| | b]pyrazin-1(2H)-yl)-1H- | | |
| | pyrazol-1-yl)-2- | | |
| | azaspiro[3.3]heptan-2- | | |
| | yl)prop-2-en-1-one | | |
| 137a/ 137b | 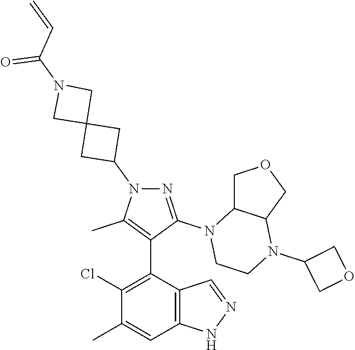 [SMILES: C=CC(=O)N1CC2(CC(n3nc(N4CCN(C5COC5)C5COCC54)c(-c4c(Cl)c(C)cc5[nH]ncc45)c3C)C2)C1] 1-(6-(4-(5-chloro-6- methyl-1H-indazol-4-yl)- 5-methyl-3-((4aS*,7aS*)- | Using Method-6 from Intermediate C36b and oxetanone (Step 1) and C-SFC-1 (mobile phase: CO₂/[IPA + 0.1% Et₃N] 72/28); Example 137a = 1^{st} eluting isomer, Example 137b = 2^{nd} eluting isomer | Example 137b: ¹H NMR (600 MHz, DMSO-d₆) δ 13.0 (s, 1H), 7.62 (s, 1H), 7.47 (s, 1H), 6.30 (m, 1H), 6.10 (m, 1H), 5.67 (m, 1H), 4.70 (m, 1H), 4.55 (t, 1H), 4.41 (t, 1H), 4.35- 4.26 (m, 4H), 4.20 (t, 1H), 4.05 (m, 1H), 3.99 (m, 1H), 3.64 (t, 1H), 3.56-3.47 (m, 2H), 3.35- 3.30 (m, 1H), 3.11 (m, 1H), 2.79-2.73 (m, 1H), 2.71-2.64 (m, 3H), 2.56-2.50 (m, 1H overlapping with DMSO peak), 2.49 (s, 3H), 2.46-2.39 (m, 2H), 2.10 (m, 1H), 1.98 (m, 3H), 1.24 (m, 1H). UPLC-MS- 2e: Rt = 3.78 min, MS m/z [M + H]⁺ 578.3/580.3; C-SFC- 3 (mobile phase: CO₂/[IPA + 0.025% NH₃] 72/28): Rt = 3.77 min. Example 137a: C-SFC-3 (mobile phase: CO₂/[IPA + 0.025% NH₃] 72/28): Rt = 2.78 min. |
| | 4-(oxetan-3- | | |
| | yl)hexahydrofuro[3,4- | | |
| | b]pyrazin-1(2H)-yl)-1H- | | |
| | pyrazol-1-yl)-2- | | |
| | azaspiro[3.3]heptan-2- | | |
| | yl)prop-2-en-1-one | | |
| 138a/ 138b | 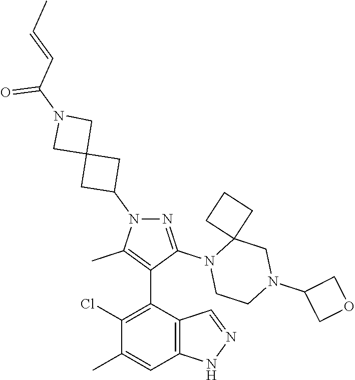 [SMILES: C/C=C/C(=O)N1CC2(CC(n3nc(N4CCN(C5COC5)CC45CCC5)c(-c4c(Cl)c(C)cc5[nH]ncc45)c3C)C2)C1] | Using Method-6 from Intermediate C28 and oxetanone (Step 1), crotonic acid (Step 3) and C-SFC- 1 (mobile phase: CO₂/[IPA + 0.1% Et₃N] 70/30); Example 138a = 1^{st} eluting isomer, Example 138b = 2^{nd} eluting isomer | Example 138b: ¹H NMR (600 MHz, DMSO-d₆) δ 12.9 (s, 1H), 7.56 (s, 1H), 7.43 (s, 1H), 6.68-6.61 (m, 1H), 5.98 (m, 1H), 4.77-4.70 (m, 1H), 4.4- 4.44 (m, 2H), 4.37-4.33 (m, 2H), 4.28 (s, 1H), 4.23 (m, 1H), 4.01 (s, 1H), 3.95 (m, 1H), 3.26 (m, 1H), 2.78-2.65 (m, 6H), 2.47 (s, 3H), 2.31- 2.19 (m, 2H), 2.19-2.08 (m, 2H), 2.03-1.93 (m, 4H), 1.93- 1-86 (m, 1H), 1.86-1.79 (m, 4H), 1.67-1.56 (m, 3H). UPLC- MS-2e: Rt = 3.81 min, MS m/z [M + H]⁺ 590.3/592.4; C-SFC- 3 (mobile phase: CO₂/[IPA + 0.025% NH₃ v/v] 70/30): Rt = 2.45 min. Example 138a: C-SFC-3 (mobile phase: CO₂/[IPA + 0.025% NH₃] 70/30): Rt = 1.73 min. |
| | (E)-1-(6-(4-(5-chloro-6- | | |
| | methyl-1H-indazol-4-yl)- | | |
| | 5-methyl-3-(8-(oxetan-3- | | |
| | yl)-5,8- | | |
| | diazaspiro[3.5]nonan-5- | | |
| | yl)-1H-pyrazol-1-yl)-2- | | |
| | azaspiro[3.3]heptan-2- | | |
| | yl)but-2-en-1-one | | |
| 139 | 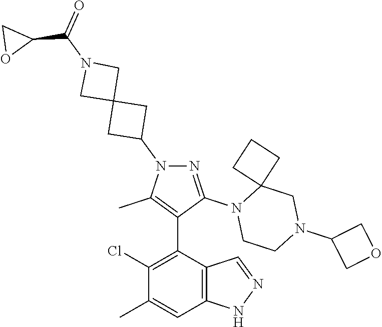 [SMILES: Cc1cc2[nH]ncc2c(-c2c(N3CCN(C4COC4)CC34CCC4)nn(C3CC4(C3)CN(C(=O)[C@@H]3CO3)C4)c2C)c1Cl] | Using Method-6 from Intermediate C28 and oxetanone (Step 1) and (2S)-oxirane- 2-carboxylic acid (Step 3) | Example 139: ¹H NMR (400 MHz, DMSO-d₆) δ 12.9 (s, 1H), 7.56 (s, 1H), 7.43 (s, 1H), 4.74 (m, 1H), 4.49-4.31 (m, 6H), 4.04 (m, 1H), 3.99 (m, 1H), 3.44 (m, 1H), 3.29-3.22 (m, 1H), 2.90-2.86 (m, 1H), 2.81-2.69 (m, 7H), 2.47 (s, 3H), 2.29-2.20 (m, 2H), 2.17- 2.08 (m, 2H), 2.01-1.77 (m, 6H), 1.67-1.54 (m, 3H). UPLC- MS-2e: Rt = 3.12 min, MS m/z [M + H]⁺ 592.3/594.3. |
| | (S)-(6-(4-(5-chloro-6- | | |
| | methyl-1H-indazol-4-yl)- | | |
| | 5-methyl-3-(8-(oxetan-3- | | |
| | yl)-5,8- | | |
| | diazaspiro[3.5]nonan-5- | | |
| | yl)-1H-pyrazol-1-yl)-2- | | |
| | azaspiro[3.3]heptan-2- | | |
| | yl)(oxiran-2- | | |
| | yl)methanone | | |
| 140a/ 140b | 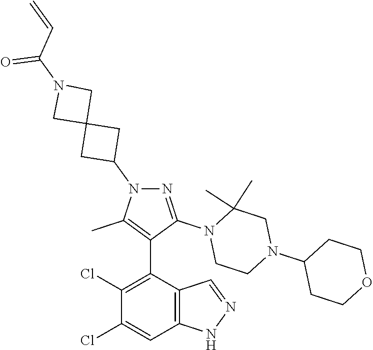 [SMILES: C=CC(=O)N1CC2(CC(n3nc(N4CCN(C5CCOCC5)CC4(C)C)c(-c4c(Cl)c(Cl)cc5[nH]ncc45)c3C)C2)C1] | Using Method-6 from Intermediate C34 and tetrahydro-4H- pyran-4-one (Step 1) and C-SFC-1 (mobile phase: CO₂/[IPA + .1% Et₃N] 73/27); Example 140a = 1^{st} eluting isomer, Example 140b = 2^{nd} eluting isomer | Example 140b: ¹H NMR (600 MHz, DMSO-d₆) δ 13.2 (s, 1H), 7.79 (s, 1H), 7.68 (s, 1H), 6.30 (m, 1H), 6.10 (m, 1H), 5.67 (m, 1H), 4.74 (m, 1H), 4.33 (s, 1H), 4.29 (s, 1H), 4.05 (s, 1H), 4.00 (s, 1H), 3.83-3.78 (m, 2H), 3.22-3.16 (m, 2H), 2.87-2.79 (m, 2H), 2.77-2.66 (m, 4H), 2.20-2.14 (m, 2H), 2.07-1.98 (m, 6H), 1.58-1.51 (m, 2H), 1.31-1.21 (m, 2H), 1.12 (s, 1.5H), 1.10 (s, 1.5H), 1.05-0.97 (m, 3H). UPLC-MS- 2e: Rt = 3.26 min, MS m/z [M + H]⁺ 612.3/614.3; C-SFC- 3 (mobile phase: CO₂/[IPA + 0.1% Et₃N] 73/27): Rt = 3.00 min. Example 140a: C-SFC-3 (mobile phase: CO₂/[IPA 0.1% Et₃N] 73/27): Rt = 2.13 min. |
| | 1-(6-(4-(5,6-dichloro-1H- | | |
| | indazol-4-yl)-3-(2,2- | | |
| | dimethyl-4-(tetrahydro- | | |
| | 2H-pyran-4-yl)piperazin- | | |
| | 1-yl)-5-methyl-1H- | | |
| | pyrazol-1-yl)-2- | | |
| | azaspiro[3.3]heptan-2- | | |
| | yl)prop-2-en-1-one | | |
| 141a/ 141b | 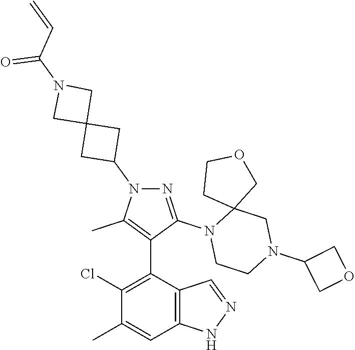 [SMILES: C=CC(=O)N1CC2(CC(n3nc(N4CCN(C5COC5)CC45CCOC5)c(-c4c(Cl)c(C)cc5[nH]ncc45)c3C)C2)C1] | Using Method-6 from Intermediate C44 and oxetanone (Step 1) and C-SFC-1 (mobile phase: CO₂/[IPA + 0.1% Et₃N] 65/35); Example 141a = 1^{st} eluting isomer, Example 141b = 2^{nd} eluting isomer | Example 141b: ¹H NMR (400 MHz, DMSO-d₆) δ 13.0 (s, 1H), 7.53 (s, 1H), 7.46 (s, 1H), 6.31 (m, 1H), 6.10 (m, 1H), 5.67 (m, 1H), 4.76 (m, 1H), 4.47-4.43 (m, 2H), 4.37-4.27 (m, 4H), 4.05 (s, 1H), 4.00 (s, 1H), 3.94-3.88 (m, 1H), 3.74- 3.60 (m, 3H), 3.30-3.24 (m, 1H), 2.85-2.68 (m, 6H), 2.47 (s, 3H), 2.22-2.05 (m, 3H), 2.00-1.91 (m, 2H), 1.97 (s, 3H), 1.74-1.63 (m, 1H). UPLC- MS-2e: Rt = 3.58 min, MS m/z [M + H]⁺ 592.3/594.3; C-SFC- 3 (mobile phase: CO₂/[IPA + 0.025% NH₃] 65/35): Rt = 2.57 min. Example 141a: C-SFC-3 (mobile phase: CO₂/[IPA + 0.025% NH₃] 65/35): Rt = 1.16 min. |
| | 1-(6-(4-(5-chloro-6- | | |
| | methyl-1H-indazol-4-yl)- | | |
| | 5-methyl-3-(9-(oxetan-3- | | |
| | yl)-2-oxa-6,9- | | |
| | diazaspiro[4.5]decan-6- | | |
| | yl)-1H-pyrazol-1-yl)-2- | | |
| | azaspiro[3.3]heptan-2- | | |
| | yl)prop-2-en-1-one | | |
| 142a/ 142b | 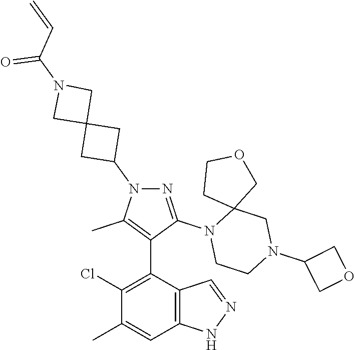 [SMILES: C=CC(=O)N1CC2(CC(n3nc(N4CCN(C5COC5)CC45CCOC5)c(-c4c(Cl)c(C)cc5[nH]ncc45)c3C)C2)C1] 1-(6-(4-(5-chloro-6- | Using Method-6 from Intermediate C45 and oxetanone (Step 1) and C-SFC-1 (mobile phase: CO₂/[IPA + 0.1% Et₃N] 70/30); Example 142a = 1^{st} eluting isomer, Example 142b = 2^{nd} eluting isomer | Example 142b: ¹H NMR (600 MHz, DMSO-d₆) δ 13.0 (s, 1H), 7.55 (s, 1H), 7.46 (s, 1H), 6.31 (m, 1H), 6.10 (m, 1H), 5.67 (m, 1H), 4.76 (m, 1H), 4.47-4.42 (m, 2H), 4.35-4.26 (m, 4H), 4.05 (m, 1H), 3.99 (m, 1H), 3.95-3.87 (m, 1H), 3.79- 3.64 (m, 3H), 3.26 (m, 1H), 2.78-2.68 (m, 5H), 2.67-2.62 (m, 1H), 2.48 (s, 3H), 2.27- 2.22 (m, 1H), 2.19-2.13 (m, 1H), 2.06-1.96 (m, 5H), 1.85- 1.71 (m, 2H). UPLC-MS-2e: Rt = 3.54 min, MS m/z [M + H]⁺ 592.3/594.3; C-SFC-3 (mobile phase: CO₂/[IPA + 0.025% NH₃] 70/30): Rt = 3.44 min. Example 142a: C-SFC-3 (mobile phase: CO₂/[IPA + 0.025% NH₃] 70/30): Rt = 1.89 min. |
| | methyl-1H-indazol-4-yl)- | | |
| | 5-methyl-3-(9-(oxetan-3- | | |
| | yl)-2-oxa-6,9- | | |
| | diazaspiro[4.5]decan-6- | | |
| | yl)-1H-pyrazol-1-yl)-2- | | |
| | azaspiro[3.3]heptan-2- | | |
| | yl)prop-2-en-1-one | | |
| 143a/ 143b | 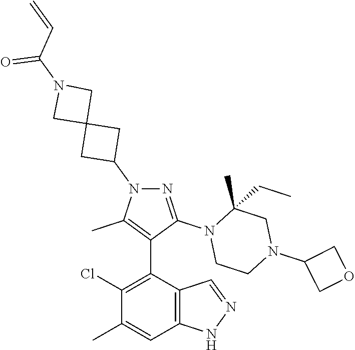 [SMILES: C=CC(=O)N1CC2(CC(n3nc(N4CCN(C5COC5)C[C@@]4(C)CC)c(-c4c(Cl)c(C)cc5[nH]ncc45)c3C)C2)C1] (R)-1-(6-(4-(5-chloro-6- methyl-1H-indazol-4-yl)- 3-(2-ethyl-2-methyl-4- | Using Method-6 from intermediate C40 and oxetanone (Step 1) and C-SFC-1 (mobile phase: CO₂/[IPA + 0.1% Et₃N] 75/25); Example 143a = 1^{st} eluting isomer, Example 143b = 2^{nd} eluting isomer | Example 143b: ¹H NMR (600 MHz, DMSO-d₆) δ 13.0 (s, 1H), 7.57 (m, 1H), 7.44 (s, 1H), 6.30 (m, 1H), 6.10 (m, 1H), 5.67 (m, 1H), 4.72 (m, 1H), 4.46 (t, 1H), 4.41 (t, 1H), 4.35 − 4.26 (m, 4H), 4.05 (s, 1H), 4.01 (m, 1H), 3.13 (m, 1H), 2.99-2.93 (m, 1H), 2.85- 2.77 (m, 1H), 2.77-2.66 (m, 4H), 2.47 (s, 3H), 2.06-1.95 (m, 2H), 1.96 (s, 3H), 1.84- 1.77 (m, 1H), 1.76-1.63 (m, 3H), 1.07-1.02 (m, 3H), 0.64- 0.58 (m, 3H). UPLC-MS-2e: Rt = 3.58 min, MS m/z [M + H]⁺ 578.3/580.3; C-SFC-3 (mobile phase: CO₂/[IPA + 0.025% NH₃] 75/25): Rt = 3.44 min. Example 143a: C-SFC-3 (mobile phase: CO₂/[IPA + 0.025% NH₃] 75/25): Rt = 2.45 min. |
| | (oxetan-3-yl)piperazin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 144a/ 144b | 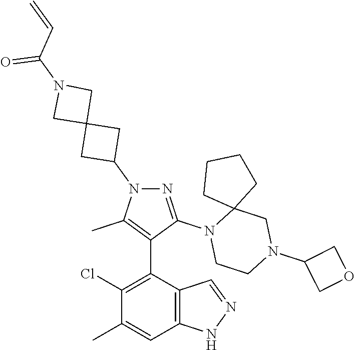 [SMILES: C=CC(=O)N1CC2(CC(n3nc(N4CCN(C5COC5)CC45CCCC5)c(-c4c(Cl)c(C)cc5[nH]ncc45)c3C)C2)C1] | Using Method-6 from C31 and oxetanone (Step 1) and C-SFC- 1 (mobile phase: 26:74 [IPA + 0.1% Et₃N]/CO₂); Example 144a = 1^{st} eluting isomer, Example 144b = 2^{nd} eluting isomer | Example 144b: ¹H NMR (600 MHz, DMSO-d₆) δ 12.9 (s, 1H), 7.53 (m, 1H), 7.44 (s, 1H), 6.30 (m, 1H), 6.11 (m, 1H), 5.67 (m, 1H), 4.75 (m, 1H), 4.44 (t, 1H), 4.41 (t, 1H), 4.33 (s, 1H), 4.33-4.28 (m, 2H), 4.27 (s, 1H), 4.05 (s, 1H), 3.98 (s, 1H), 3.18 (m, 1H), 2.81-2.65 (m, 6H), 2.52 (m, 2H), 2.48 (s, 3H), 2.06-1.93 (m, 4H), 1.98 (s, 3H), 1.87 (m, 2H), 1.62 (m, 2H), 1.48-1.38 (m, 2H). UPLC-MS-4: Rt = 0.79 min, MS m/z [M + H]⁺ 590.5/592.5; C-SFC-3 (mobile phase: 26:74 [IPA + 0.025% NH₃]/CO₂): Rt = 3.69 min. Example 144a: C- SFC-3 (mobile phase: 26:74 [IPA + 0.025% NH₃]/CO₂): Rt = 2.59 min. |
| | 1-(6-(4-(5-chloro-6- | | |
| | methyl-1H-indazol-4-yl)- | | |
| | 5-methyl-3-(9-(oxetan-3- | | |
| | yl)-6,9- | | |
| | diazaspiro[4.5]decan-6- | | |
| | yl)-1H-pyrazol-1-yl)-2- | | |
| | azaspiro[3.3]heptan-2- | | |
| | yl)prop-2-en-1-one | | |
| 145a/ 145b | 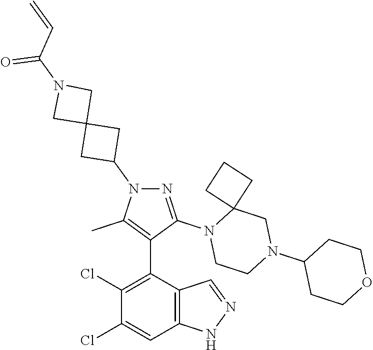 [SMILES: C=CC(=O)N1CC2(CC(n3nc(N4CCN(C5CCOCC5)CC45CCC5)c(-c4c(Cl)c(Cl)cc5[nH]ncc45)c3C)C2)C1] | Using Method-6a from Intermediate C33 and tetrahydro- 4H-pyran-4-one (Step 1) and C-SFC- 4 (mobile phase: CO₂/[IPA + 0.1% Et₃N] 70/30); Example 145a = 1^{st} eluting isomer, Example 145b = 2^{nd} eluting isomer | Example 145b: ¹H NMR (600 MHz, DMSO-d₆) δ 13.2 (s, 1H), 7.76 (s, 1H), 7.65 (s, 1H), 6.30 (m, 1H), 6.09 (m, 1H), 5.67 (m, 1H), 4.76 (m, 1H), 4.33 (s, 1H), 4.28 (s, 1H), 4.05 (s, 1H), 4.00 (s, 1H), 3.82 (m, 2H), 3.21 (m, 2H), 2.78-2.76 (m, 6H), 2.48-2.41 (m, 1H), 2.32-2.06 (m, 6H), 2.01 (s, 3H), 1.72 (m, 1H), 1.62-1.50 (m, 5H), 1.35-1.26 (m, 2H). UPLC-MS-4: Rt = 3.26 min, MS m/z [M + H]⁺ 624.3/626.3/ 628.3; C-SFC-3 (mobile phase: CO₂/[IPA + 0.1% Et₃N] 70/30): Rt = 2.95 min. Example 145a: C-SFC-3 (mobile phase: CO₂/[IPA + 0.1% Et₃N] 70/30): Rt = 1.71 min. |
| | 1-(6-(4-(5,6-dichloro-1H- | | |
| | indazol-4-yl)-5-methyl-3- | | |
| | (8-(tetrahydro-2H-pyran- | | |
| | 4-yl)-5,8- | | |
| | diazaspiro[3.5]nonan-5- | | |
| | yl)-1H-pyrazol-1-yl)-2- | | |
| | azaspiro[3.3]heptan-2- | | |
| | yl)prop-2-en-1-one | | |
| 146a/ 146b | 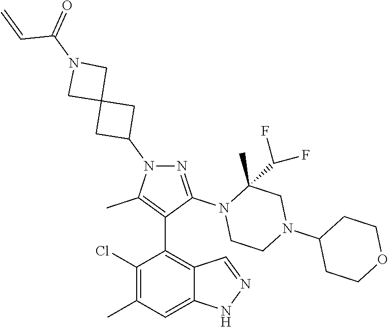 [SMILES: C=CC(=O)N1CC2(CC(n3nc(N4CCN(C5CCOCC5)C[C@@]4(C)C(F)F)c(-c4c(Cl)c(C)cc5[nH]ncc45)c3C)C2)C1] (S)-1-(6-(4-(5-chloro-6- methyl-1H-indazol-4-yl)-3-(2-(difluoromethyl)-2- methyl-4-(tetrahydro-2H-pyran-4-yl)piperazin-1- yl)-5-methyl-1H-pyrazol-1-yl)-2- azaspiro[3.3]heptan-2-yl)prop-2-en-1-one | Using Method-6a from Intermediate C43 and tetrahydro- 4H-pyran-4-one (Step 1) and C-SFC- 35 (mobile phase: CO₂/[IPA + 0.1% Et₃N] 70/30); Example 146a = 1^{st} eluting isomer, Example 146b = 2^{nd} eluting isomer | Example 146b: ¹H NMR (400 MHz, DMSO-d₆) δ 13.02 (s, 1H), 7.58 (s, 1H), 7.47 (s, 1H), 6.67-6.32 (m, 1H), 6.38-6.24 (m, 1H), 6.15-6.03 (m, 1H), 5.72-5.62 (m, 1H), 4.81-4.68 (m, 1H), 4.33 (s, 1H), 4.29 (s, 1H), 4.05 (s, 1H), 4.00 (s, 1H), 3.87-3.77 (m, 2H), 3.25-3.15 (m, 2H), 3.15-3.05 (m, 1H), 2.88-2.77 (m, 1H), 2.77-2.68 (m, 4H), 2.48 (s, 3H), 2.42- 2.29 (m, 1H), 2.29-2.19 (m, 1H), 2.10-2.00 (m, 1H), 1.96 (s, 3H), 1.60-1.51 (m, 2H), 1.36-1.19 (m, 4H), 1.07-0.99 (m, 3H); UPLC-MS-4: Rt = 0.73 min, MS m/z [M + H]⁺ 628.5/630.5 ; C-SFC-3 (mobile phase: CO₂/[IPA + 0.025% NH₃] 70/30): Rt 2.07 min. Example 146a: C-SFC-3 (mobile phase: CO₂/[IPA + 0.025% NH₃] 70/30): Rt = 1.30 min. |
| 147a/ 147b | 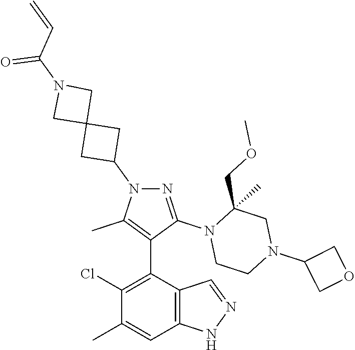 [SMILES: C=CC(=O)N1CC2(CC(n3nc(N4CCN(C5COC5)C[C@]4(C)COC)c(-c4c(Cl)c(C)cc5[nH]ncc45)c3C)C2)C1] (R)-1-(6-(4-(5-chloro-6- methyl-1H-indazol-4-yl)- | Using Method-6 from Intermediate C47 and oxetanone (Step 1) and C-SFC-2 (mobile phase: CO₂/[MeOH + 0.025% NH₃] 74/26); Example 147a = 1^{st} eluting isomer, Example 147b = 2^{nd} eluting isomer | Example 147b: ¹H NMR (400 MHz, DMSO-d₆) δ 12.9 (s, 1H), 7.74 (m, 1H), 7.44 (s, 1H), 6.30 (m, 1H), 6.11 (m, 1H), 5.67 (m, 1H), 4.74 (m, 1H), 4.43 (m, 2H), 4.34 (s, 1H), 4.32 (s, 1H), 4.32-4.24 (m, 2H), 4.06 (s, 1H), 4.03 (s, 1H), 3.72 (m, 1H), 3.44 (dd, 1H), 3.20-3.11 (m, 4H), 2.89 (m, 2H), 2.78-2.65 (m, 4H), 2.48 (s, 3H), 2.10 (m, 1H), 1.98 (s, 3H), 1.94 (m, 1H), 1.81 (m, 1H), 1.71 (m, 1H), 1.09 (m, 3H). UPLC-MS-4: Rt = 0.78 min, MS m/z [M + H]⁺ 594.4/596.4; C-SFC-3 (mobile phase: CO₂/[MeOH + 0.025% NH₃] 72/28): Rt = 1.99 min. Example 147a: C-SFC-3 (mobile phase: CO₂/[MeOH + 0.025% NH₃] 72/28): Rt = 1.58 min. |
| | 3-(2-(methoxymethyl)-2- | | |
| | methyl-4-(oxetan-3- | | |
| | yl)piperazin-1-yl)-5- | | |
| | methyl-1H-pyrazol-1-yl)- | | |
| | 2-azaspiro[3.3]heptan-2- | | |
| | yl)prop-2-en-1-one | | |
| 148a/ 148b | 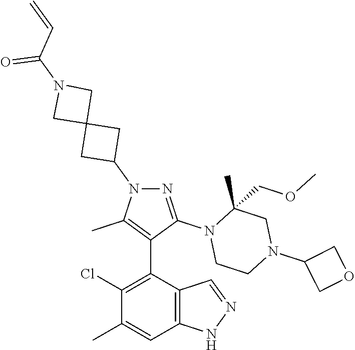 [SMILES: C=CC(=O)N1CC2(CC(n3nc(N4CCN(C5COC5)C[C@@]4(C)COC)c(-c4c(Cl)c(C)cc5[nH]ncc45)c3C)C2)C1] | Using Method-6 from Intermediate C46 and oxetanone (Step 1) and C-SFC-4 (mobile phase: CO₂/[IPA + 0.025% NH₃] 75/25); Example 148a = 1^{st} eluting isomer, Example 148b = 2^{nd} eluting isomer | Example 148b: ¹H NMR (400 MHz, DMSO-d₆) δ 13.0 (s, 1H), 7.57 (m, 1H), 7.46 (s, 1H), 6.30 (m, 1H), 6.10 (m, 1H), 5.68 (m, 1H), 4.76 (m, 1H), 4.46 (t, 1H), 4.41 (t, 1H), 4.34 (s, 1H), 4.32 (s, 1H), 4.32-4.24 (m, 2H), 4.06 (s, 1H), 4.04 (s, 1H), 3.61-3.55 (m, 1H), 3.29 (m, 1H), 3.19- 3.12 (m, 1H), 3.17 (s, 3H), 2.99-2.86 (m, 2H), 2.79-2.65 (m, 4H), 2.48 (s, 3H), 2.10- 2.04 (m, 1H), 2.03-1.96 (m, 1H), 1.98 (s, 3H), 1.77-1.69 (m, 2H), 1.13 (s, 1.5H), 1.12 (s, 1.5H). UPLC-MS-4: Rt = 0.77 min, MS m/z [M + H]⁺ 594.5/596.5; C-SFC-3 (mobile phase: CO₂/[IPA + 0.1% Et₃N] 75/25): Rt = 3.35 min. Example 148a: |
| | (S)-1-(6-(4-(5-chloro-6- | | C-SFC-3 (mobile phase: |
| | methyl-1H-indazol-4-yl)- | | CO₂/[IPA + 0.1% Et₃N] 75/25): |
| | 3-(2-(methoxymethyl)-2- | | Rt = 2.35 min. |
| | methyl-4-(oxetan-3- | | |
| | yl)piperazin-1-yl)-5- | | |
| | methyl-1H-pyrazol-1-yl)- | | |
| | 2-azaspiro[3.3]heptan-2- | | |
| | yl)prop-2-en-1-one | | |
| 149a/ 149b | 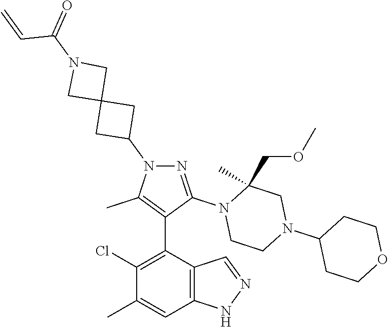 [SMILES: C=CC(=O)N1CC2(CC(n3nc(N4CCN(C5CCOCC5)C[C@]4(C)COC)c(-c4c(Cl)c(C)cc5[nH]ncc45)c3C)C2)C1] (R)-1-(6-(4-(5-chloro-6- methyl-1H-indazol-4-yl)- 3-(2-(methoxymethyl)-2- | Using Method-6 from Intermediate C47 and tetrahydro-4H- pyran-4-one (Step 1) and C-SFC-20 (mobile phase: CO₂/[IPA + 0.1% Et₃N] 70/30); Example 149a = 1^{st} eluting isomer, Example 149b = 2^{nd} eluting isomer | Example 149b: ¹H NMR (400 MHz, DMSO-d₆) δ 12.9 (s, 1H), 7.75 (s, 1H), 7.44 (s, 1H), 6.30 (m, 1H), 6.10 (m, 1H), 5.67 (m, 1H), 4.73 (m, 1H), 4.33 (s, 1H), 4.30 (s, 1H), 4.05 (s, 1H), 4.02 (s, 1H), 3.80 (m, 2H), 3.72 (m, 1H), 3.44 (t, 1H), 3.19 (m, 2H), 3.13 (s, 3H), 2.89-2.63 (m, 6H), 2.47 (s, 3H), 2.35 (m, 1H), 2.22-2.10 (m, 2H), 2.06-1.93 (m, 2H), 1.97 (s, 3H), 1.54 (m, 2H), 1.30-1.18 (m, 2H), 1.03 (m, 3H). UPLC-MS-4: Rt = 0.74 min; MS m/z [M + H]⁺ 622.5/ 624.5; C-SFC-3 (mobile phase: CO₂/[IPA + 0.025% NH₃] 76/26): Rt = 3.80 min, Example 149a: C-SFC-3 (mobile phase: CO₂/[IPA + 0.025% NH₃] 76/26): Rt = 3.19 min. |
| | methyl-4-(tetrahydro-2H- | | |
| | pyran-4-yl)piperazin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 150a/ 150b | 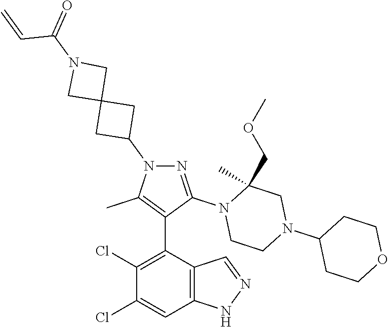 [SMILES: C=CC(=O)N1CC2(CC(n3nc(N4CCN(C5CCOCC5)C[C@]4(C)COC)c(-c4c(Cl)c(Cl)cc5[nH]ncc45)c3C)C2)C1] (R)-1-(6-(4-(5,6-dichloro- 1H-indazol-4-yl)-3-(2- (methoxymethyl)-2- | | Example 150b: ¹H NMR (400 MHz, DMSO-d₆) δ 13.2 (s, 1H), 7.87 (s, 1H), 7.79 (s, 1H), 6.30 (m, 1H), 6.10 (m, 1H), 5.67 (m, 1H), 4.75 (m, 1H), 4.33 (s, 1H), 4.30 (s, 1H), 4.05 (s, 1H), 4.02 (s, 1H), 3.80 (m, 2H), 3.72 (m, 1H), 3.39 (t, 1H), 3.19 (m, 2H), 3.12 (s, 3H), 2.85 (m, 2H), 2.79-2.63 (m, 4H), 2.37 (m, 1H), 2.23-2.13 (m, 2H), 2.06-1.91 (m, 2H), 1.99 (s, 3H), 1.55 (m, 2H), 1.32-1.17 (m, 2H), 1.02 (m, 3H). UPLC-MS-4: Rt = 0.69 min; MS m/z [M + H]⁺ 642.5/ 644.6; C-SFC-3 (mobile phase: CO₂/[IPA + 0.1% Et₃N] 75/25): Rt = 3.20 min, Example 150a: C-SFC-3 (mobile phase: CO₂/[IPA + 0.1% Et₃N] 75/25): Rt = 2.61 min. |
| | methyl-4-(tetrahydro-2H- | | |
| | pyran-4-yl)piperazin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 151a/ 151b | 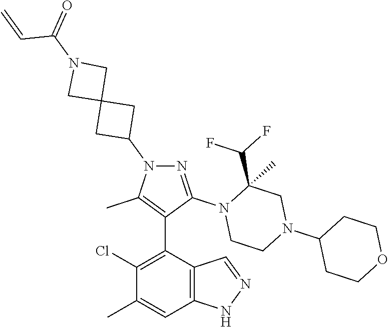 [SMILES: C=CC(=O)N1CC2(CC(n3nc(N4CCN(C5CCOCC5)C[C@]4(C)C(F)F)c(-c4c(Cl)c(C)cc5[nH]ncc45)c3C)C2)C1] (R)-1-(6-(4-(5-chloro-6- methyl-1H-indazol-4-yl)- 3-(2-(difluoromethyl)-2- methyl-4-(tetrahydro-2H- | Using Method-6a from Intermediate C42 and tetrahydro- 4H-pyran-4-one (Step 1) and C-SFC- 4 (mobile phase: CO₂/[IPA + 0.1% Et₃N] 73/27); Example 151a = 1^{st} eluting isomer, Example 151b = 2^{nd} eluting isomer | Example 151b: ¹H NMR (400 MHz, DMSO-d₆) δ 12.98 (s, 1H), 7.64 (s, 1H), 7.47 (s, 1H), 6.69 (td, 1H), 6.30 (m, 1H), 6.09 (m, 1H), 5.67 (m, 1H), 4.73 (m, 1H), 4.33 (s, 1H), 4.29 (s, 1H), 4.05 (s, 1H), 4.00 (s, 1H), 3.84-3.74 (m, 2H), 3.25-3.13 (m, 2H), 2.99-2.80 (m, 2H), 2.79-2.63 (m, 4H), 2.48 (s, 3H), 2.49-2.45 (m, 1H), 2.28-2.18 (m, 3H), 1.97 (s, 3H), 2.00-1.90 (m, 1H), 1.60-1.47 (m, 2H), 1.32-1.21 (m, 2H), 1.15 (s, 1.5H), 1.13 (s, 1.5H). UPLC-MS-4: Rt = 0.76 min, MS m/z [M + H]⁺ 628.5/630.5; C-SFC-3 (mobile phase: CO₂/[IPA + 0.1% NH₃] 73/27): Rt = 3.02 min. Example 151a: C-SFC-3 (mobile phase: CO₂/[IPA + 0.1% NH₃] 73/27): Rt = 1.89 min. |
| | pyran-4-yl)piperazin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 152a/ 152b | 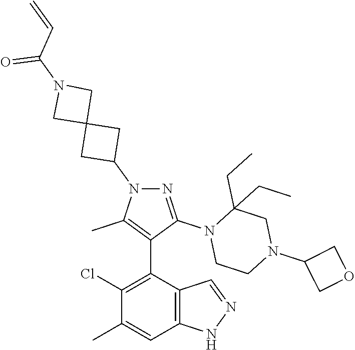 [SMILES: C=CC(=O)N1CC2(CC(n3nc(N4CCN(C5COC5)CC4(CC)CC)c(-c4c(Cl)c(C)cc5[nH]ncc45)c3C)C2)C1] | Using Method-6 from C30 and oxetanone (Step 1) and C-SFC- 1 (mobile phase: CO₂/[IPA + 0.1% Et₃N] 75/25); Example 152a = 1^{st} eluting isomer, Example 152b = 2^{nd} eluting isomer | Example 152b: ¹H NMR (400 MHz, DMSO-d₆) δ 12.97 (s, 1H), 7.57 (m, 1H), 7.44 (s, 1H), 6.30 (m, 1H), 6.10 (m, 1H), 5.67 (m, 1H), 4.73 (m, 1H), 4.50-4.43 (m, 2H), 4.33 (s, 1H), 4.29 (s, 1H), 4.33-4.28 (m, 2H), 4.04 (s, 1H), 4.01 (s, 1H), 3.15 (m, 1H), 2.94-2.87 (m, 2H), 2.77-2.69 (m, 4H), 2.47 (s, 3H), 2.07-1.98 (m, 2H), 1.93 (s, 3H), 1.90-1.49 (m, 6H), 0.62 (m, 3H), 0.53 (m, 3H). UPLC-MS-3: Rt = 0.79 min, MS m/z [M + H]⁺ 592.5/ 594.4; C-SFC-3 (mobile phase: CO₂/[IPA + 0.1% Et₃N] 75/25): Rt = 3.27 min. Example 152a: C-SFC-3 (mobile phase: CO₂/[IPA + 0.1% Et₃N] 75/25): Rt = 2.50 min. |
| | 1-(6-(4-(5-chloro-6- | | |
| | methyl-1H-indazol-4-yl)- | | |
| | 3-(2,2-diethyl-4-(oxetan- | | |
| | 3-yl)piperazin-1-yl)-5- | | |
| | methyl-1H-pyrazol-1-yl)- | | |
| | 2-azaspiro[3.3]heptan-2- | | |
| | yl)prop-2-en-1-one | | |
| 153a/ 153b | 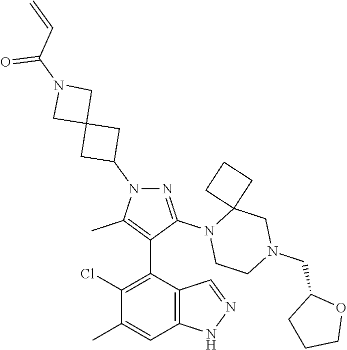 [SMILES: C=CC(=O)N1CC2(CC(n3nc(N4CCN(C[C@H]5CCCO5)CC45CCC5)c(-c4c(Cl)c(C)cc5[nH]ncc45)c3C)C2)C1] (R)-1-(6-(4-(5-chloro-6- | Using Method-6 from Intermediate C28 and Intermediate B5 (Step 1) and C-SFC- 4 (mobile phase: CO₂/[IPA + 0.025% NH₃]70/30); Example 153a = 1^{st} eluting isomer, Example 153b = 2^{nd} eluting isomer | Example 153b: ¹H NMR (400 MHz, DMSO-d₆) δ 12.97 (s, 1H), 7.57 (m, 1H), 7.44 (s, 1H), 6.30 (m, 1H), 6.10 (m, 1H), 5.67 (m, 1H), 4.74 (m, 1H), 4.35 (s, 1H), 4.29 (s, 1H), 4.06 (s, 1H), 4.00 (s, 1H), 3.86 (p, 1H), 3.69 (m, 1H), 3.56 (m, 1H), 2.81-2.61 (m, 6H), 2.48 (s, 3H), 2.35-2.19 (m, 4H), 2.18-2.07 (m, 3H), 1.99 (s, 3H), 1.92-1.84 (m, 1H), 1.81- 1.69 (m, 3H), 1.64-1.54 (m, 3H), 1.50-1.42 (m, 1H). UPLC- MS-4: Rt = 0.76 min, MS m/z [M + H]⁺ 604.5/606.5; C-SFC- 3 (mobile phase: CO₂/[IPA + 0.025% NH₃] 70/30): Rt = 2.36 min. Example 153a: C-SFC-3 (mobile phase: CO₂/[IPA + 0.025% NH₃] 70/30): Rt = 1.66 min. |
| | methyl-1H-indazol-4-yl)- | | |
| | 5-methyl-3-(8- | | |
| | ((tetrahydrofuran-2- | | |
| | yl)methyl)-5,8- | | |
| | diazaspiro[3.5]nonan-5- | | |
| | yl)-1H-pyrazol-1-yl)-2- | | |
| | azaspiro[3.3]heptan-2- | | |
| | yl)prop-2-en-1-one | | |
| 154a/ 154b | 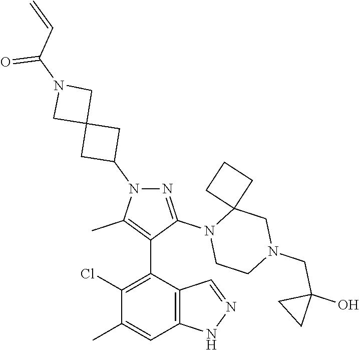 [SMILES: C=CC(=O)N1CC2(CC(n3nc(N4CCN(CC5(O)CC5)CC45CCC5)c(-c4c(Cl)c(C)cc5[nH]ncc45)c3C)C2)C1] 1-(6-(4-(5-chloro-6- | Using Method-6 from Intermediate C28 and B6 (Step 1) and C-SFC-1 (mobile phase: CO₂/[IPA + 0.1% Et₃N] 65/35); Example 154a = 1^{st} eluting isomer, Example 154b = 2^{nd} eluting isomer | Example 154b: ¹H NMR (600 MHz, DMSO-d₆) δ 12.9 (s, 1H), 7.55 (s, 1H), 7.43 (s, 1H), 6.30 (m, 1H), 6.10 (m, 1H), 5.67 (m, 1H), 4.80-4.69 (m, 2H), 4.33 (s, 1H), 4.29 (s, 1H), 4.06 (s, 1H), 4.00 (s, 1H), 2.82-2.64 (m, 6H), 2.47 (s, 3H), 2.42-2.30 (m, 2H), 2.37- 2.33 (m, 1H), 2.31-2.18 (m, 4H), 2.17-2.05 (m, 2H), 1.98 (s, 3H), 1.88-1.78 (m, 1H), 1.66-1.53 (m, 3H), 0.51 (m, 2H), 0.33 (m, 2H). UPLC-MS- 2e: Rt = 3.37 min, MS m/z [M + H]⁺ 590.3/592.3; C-SFC- 3 (mobile phase: CO₂/[IPA + 0.025% NH₃] 65/35): Rt = 2.87 min. Example 154a: C-SFC-3 (mobile phase: CO₂/[IPA + 0.025% NH₃ v/v] 65/35): Rt = 1.15 min. |
| | methyl-1H-indazol-4-yl)- | | |
| | 3-(8-((1- | | |
| | hydroxycyclopropyl)meth | | |
| | yl)-5,8- | | |
| | diazaspiro[3.5]nonan-5- | | |
| | yl)-5-methyl-1H-pyrazol- | | |
| | 1-yl)-2- | | |
| | azaspiro[3.3]heptan-2- | | |
| | yl)prop-2-en-1-one | | |
| 155a/ 155b | 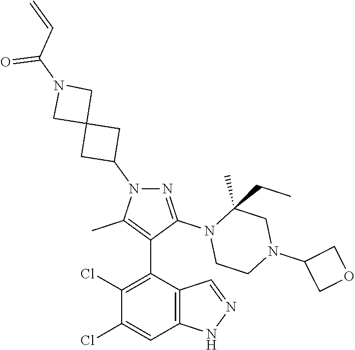 [SMILES: C=CC(=O)N1CC2(CC(n3nc(N4CCN(C5COC5)C[C@]4(C)CC)c(-c4c(Cl)c(Cl)cc5[nH]ncc45)c3C)C2)C1] | Using Method-6 from Intermediate C35 and oxetanone (Step 1) and C-SFC-1 (mobile phase: CO₂/IPA 70/30); Example 155a = 1^{st} eluting isomer, Example 155b = 2^{nd} eluting isomer | Example 155b: ¹H NMR (600 MHz, DMSO-d₆) δ 13.25 (s, 1H), 7.79 (s, 1H), 7.67 (s, 1H), 6.30 (m, 1H), 6.10 (m, 1H), 5.67 (m, 1H), 4.75 (m, 1H), 4.51-4.40 (m, 2H), 4.35-4.26 (m, 2H), 4.33 (s, 1H), 4.29 (s, 1H), 4.05 (s, 1H), 4.01 (s, 1H), 3.16 (m, 1H), 3.07 (m, 1H), 2.87 (m, 1H), 2.80-2.63 (m, 4H), 2.07-1.90 (m, 6H), 1.79 (m, 1H), 1.64 (m, 2H), 0.88 (s, 3H), 0.62 (m, 3H). UPLC-MS- 4: Rt = 0.84 min, MS m/z [M + H]⁺ 598.4/600.4/602.4; C-SFC-3 (mobile phase: CO₂/IPA 70/30): Rt = 2.28 min. Example 155a: C-SFC-3 (mobile phase: CO₂/IPA 70/30): Rt = 1.43 min. |
| | (S)-1-(6-(4-(5,6-dichloro- | | |
| | 1H-indazol-4-yl)-3-(2- | | |
| | ethyl-2-methyl-4-(oxetan- | | |
| | 3-yl)piperazin-1-yl)-5- | | |
| | methyl-1H-pyrazol-1-yl)- | | |
| | 2-azaspiro[3.3]heptan-2- | | |
| | yl)prop-2-en-1-one | | |
| 156a/ 156b | 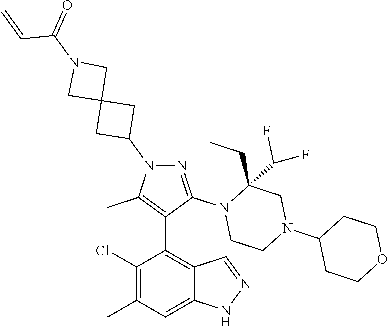 [SMILES: C=CC(=O)N1CC2(CC(n3nc(N4CCN(C5CCOCC5)C[C@@]4(CC)C(F)F)c(-c4c(Cl)c(C)cc5[nH]ncc45)c3C)C2)C1] (S)-1-(6-(4-(5-chloro-6- methyl-1H-indazol-4-yl)- 3-(2-(difluoromethyl)-2- ethyl-4-(tetrahydro-2H- | Using Method-6 from Intermediate C48 and tetrahydro-4H- pyran-4-one (Step 1) and C-SFC-4 (mobile phase: CO₂/[IPA + 0.1% Et₃N] 67/33); Example 156a = 1^{st} eluting isomer, Example 156b = 2^{nd} eluting isomer | Example 156b: ¹H NMR (400 MHz, DMSO-d₆) δ 13.04 (s, 1H), 7.59 (s, 1H), 7.48 (s, 1H), 6.54 (m, 1H), 6.30 (m, 1H), 6.09 (m, 1H), 5.67 (m, 1H), 4.73 (m, 1H), 4.33 (s, 1H), 4.28 (s, 1H), 4.04 (s, 1H), 4.00 (s, 1H), 3.81 (d, 2H), 3.20 (t, 2H), 3.11 (m, 1H), 2.87 (m, 1H), 2.80-2.59 (m, 5H), 2.48 (s, 3H), 2.39 (m, 1H), 2.24 (m, 1H), 2.12 (m, 1H), 1.97 (m, 1H), 1.92 (s, 3H), 1.68 (m, 2H), 1.62-1.54 (m, 2H), 1.35- 1.23 (m, 2H), 0.62 (q, 3H). UPLC-MS-4: Rt = 0.75 min, MS m/z [M + H]⁺ 642.4/644.4; C-SFC-3 (mobile phase: CO₂/[IPA + 0.025% NH₃] 67/33): Rt = 2.74 min. Example 156a: C-SFC-3 (mobile phase: CO₂/[IPA + 0.025% NH₃] 67/33): Rt = 0.93 min. |
| | pyran-4-yl)piperazin-1- | | |
| | yl)-5-methyl-1H-pyrazol- | | |
| | 1-yl)-2- | | |
| | azaspiro[3.3]heptan-2- | | |
| | yl)prop-2-en-1-one | | |
| 157a/ 157b/ 157c/ 157d | 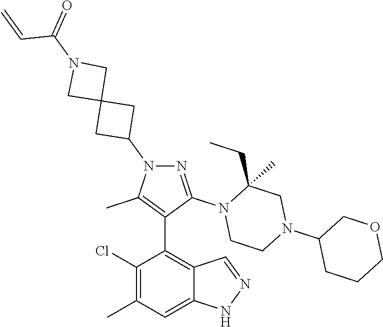 [SMILES: C=CC(=O)N1CC2(CC(n3nc(N4CCN(C5CCCOC5)C[C@]4(C)CC)c(-c4c(Cl)c(C)cc5[nH]ncc45)c3C)C2)C1] 1-(6-(4-(5-chloro-6- methyl-1H-indazol-4-yl)- 3-((2S)-2-ethyl-2-methyl- 4-(tetrahydro-2H-pyran- 3-yl)piperazin-1-yl)-5- methyl-1H-pyrazol-1-yl)- 2-azaspiro[3.3]heptan-2- yl)prop-2-en-1-one | Using Method-6 from Intermediate C39 and dihydro-2H- pyran-3(4H)-one (Step 1) and C- HPLC-8 (mobile phase: [n- heptane/IPA 75/25] + 0.1%Et₃N); Example 157a = 1^{st} eluting isomer, Example 157b = 2^{nd} eluting isomer, Example 157c = 3^{rd} eluting isomer, Example 157d = 4^{th} eluting isomer | Example 157c: ¹H NMR (400 MHz, DMSO-d₆) δ 12.96 (s, 1H), 7.57 (s, 1H), 7.44 (s, 1H), 6.30 (m, 1H), 6.09 (m, 1H), 5.67 (m, 1H), 4.72 (m, 1H), 4.32 (s, 1H), 4.28 (s, 1H), 4.04 (s, 1H), 4.00 (s, 1H), 3.76 (d, 1H), 3.66 (d, 1H), 3.12 (m, 1H), 3.09-2.91 (m, 2H), 2.83- 2.59 (m, 5H), 2.47 (s, 3H), 2.25 (m, 1H), 2.22-2.14 (m, 2H), 2.08 (m, 1H), 1.95 (s, 3H), 1.92 (m, 1H), 1.87-1.74 (m, 2H), 1.68-1.56 (m, 2H), 1.40 (m, 1H), 1.27 (m, 1H), 0.89 (m, 3H), 0.64 (m, 3H). UPLC-MS-4: Rt = 0.72 min, MS m/z [M + H]⁺ 606.5/608.5; C-SFC-3 (mobile phase: CO₂/[IPA + 0.025% NH₃] 70/30): Rt = 2.70 min. Example 157d: ¹H NMR (400 MHz, DMSO-d₆) δ 12.97 (s, 1H), 7.57 (s, 1H), 7.44 (s, 1H), 6.30 (m, 1H), 6.09 (m, 1H), 5.67 (m, 1H), 4.72 (m, 1H), 4.32 (s, 1H), 4.28 (s, 1H), 4.04 (s, 1H), 4.00 (s, 1H), 3.75 (d, 1H), 3.66 (d, 1H), 3.13 (m, 1H), 3.06 (m, 1H), 2.98 (m, |
| | | | 1H), 2.80-2.63 (m, 5H), 2.47 |
| | | | (s, 3H), 2.26 (m, 1H), 2.19 (m, |
| | | | 1H), 2.14-2.07 (m, 2H), 1.95 |
| | | | (s, 3H), 1.90 (m, 1H), 1.86- |
| | | | 1.76 (m, 2H), 1.67-1.53 (m, |
| | | | 2H), 1.39 (m, 1H), 1.22 (m, |
| | | | 1H), 0.90 (m, 3H), 0.64 (m, |
| | | | 3H). UPLC-MS-4: Rt = 0.76 |
| | | | min, MS m/z [M + H]⁺ 606.5/ |
| | | | 608.5; C-SFC-3 (mobile |
| | | | phase: CO₂/[IPA + 0.025% |
| | | | NH₃] 70/30): Rt = 3.10 min. |
| | | | Example 157a: C-SFC-3 |
| | | | (mobile phase: |
| | | | CO₂/[IPA + 0.025% NH₃] |
| | | | 70/30): Rt = 1.60 min. |
| | | | Example 157b: C-SFC-3 |
| | | | (mobile phase: |
| | | | CO₂/[IPA + 0.025% NH₃] |
| | | | 70/30): Rt = 1.80 min. |
| 158a/ 158b | 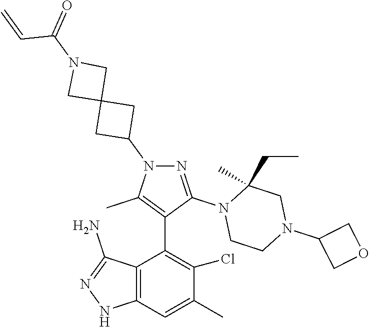 [SMILES: C=CC(=O)N1CC2(CC(n3nc(N4CCN(C5COC5)C[C@]4(C)CC)c(-c4c(Cl)c(C)cc5[nH]nc(N)c45)c3C)C2)C1] | Using Method-6 from Intermediate C41 and oxetan-3-one (Step 1) and C-SFC- 7 (mobile phase: CO₂/[MeOH + 0.1% NH₃] 55/45); Example 158a = 1^{st} eluting isomer, Example 158b = 2^{nd} eluting isomer | Example 158a: UPLC-MS-2e: Rt = 3.42 min; MS m/z [M + H]⁺ 593.5 − 595.4; C-SFC-8 (mobile phase: CO₂/[MeOH + 0.1% NH₃] 55/45): Rt = 2.34 min, Example 158b: C-SFC-8 (mobile phase: CO₂/[MeOH + 0.1% NH₃] 55/45): Rt = 3.66 min. |
| | (S)-1-(6-(4-(3-amino-5- | | |
| | chloro-6-methyl-1H- | | |
| | indazol-4-yl)-3-(2-ethyl-2- | | |
| | methyl-4-(oxetan-3- | | |
| | yl)piperazin-1-yl)-5- | | |
| | methyl-1H-pyrazol-1-yl)- | | |
| | 2-azaspiro[3.3]heptan-2- | | |
| | yl)prop-2-en-1-one | | |
| 159a/ 159b | 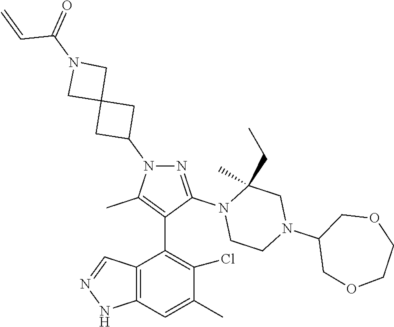 [SMILES: C=CC(=O)N1CC2(CC(n3nc(N4CCN(C5COCCOC5)C[C@]4(C)CC)c(-c4c(Cl)c(C)cc5[nH]ncc45)c3C)C2)C1] | Using Method-6 from Intermediate C39 and 1,4-dioxepan-6- one [28544-93-6] (Step 1) and C-SFC- 34 (mobile phase: CO₂/[IPA + 0.1% Et₃N] 70/30); Example 159a = 1^{st} eluting isomer, Example 159b = 2^{nd} eluting isomer | Example 159b: UPLC-MS-2e: Rt = 3.42 min; MS m/z [M + H]⁺ 622.5/624.5; C-SFC-3 (mobile phase: CO₂/[IPA + 0.1% NH₃] 75/25): Rt = 3.60 min, Example 159a: C-SFC-3 (mobile phase: CO₂/[IPA + 0.1% NH₃] 75/25): Rt = 2.97 min. |
| | (S)-1-(6-(3-(4-(1,4- | | |
| | dioxepan-6-yl)-2-ethyl-2- | | |
| | methylpiperazin-1-yl)-4- | | |
| | (5-chloro-6-methyl-1H- | | |
| | indazol-4-yl)-5-methyl- | | |
| | 1H-pyrazol-1-yl)-2- | | |
| | azaspiro[3.3]heptan-2- | | |
| | yl)prop-2-en-1-one | | |

### Method-7 for the Preparation of Example 160a and 160b: 1-(6-(4-(5-Chloro-6-methyl-1H-indazol-4-yl)-3-(8-(2-hydroxy-2-methylpropyl)-5,8-diazaspiro[3.5]nonan-5-yl)-5-methyl-1H-pyrazol-1-yl)-2-azaspiro[3.3]heptan-2-yl)prop-2-en-1-one

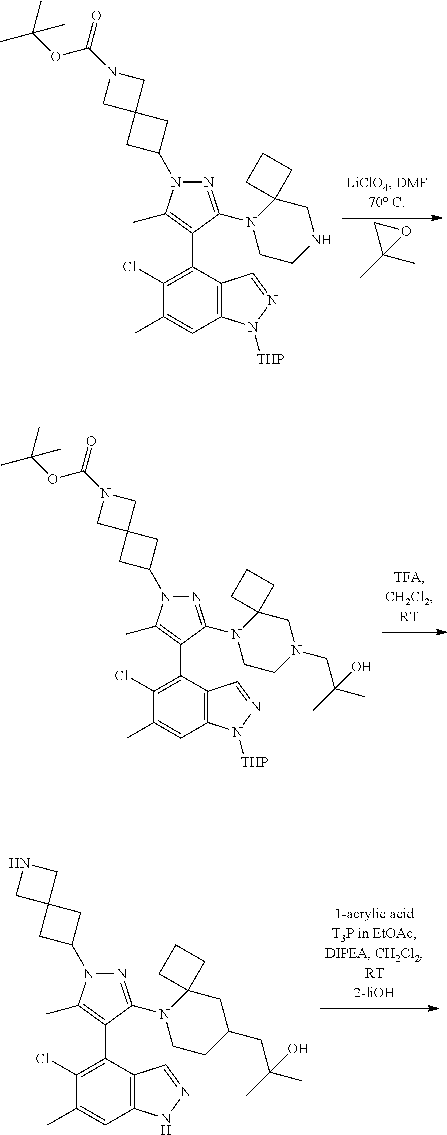

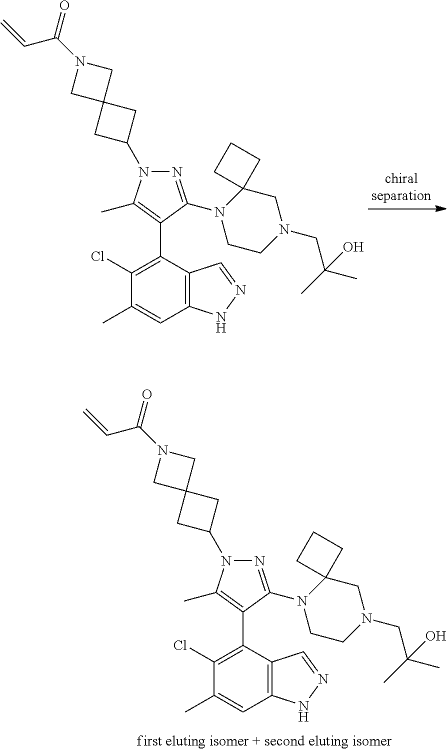

### Step 1: Tert-butyl 6-(4-(5-chloro-6-methyl-1-(tetrahydro-2H-pyran-2-yl)-1H-indazol-4-yl)-3-(8-(2-hydroxy-2-methylpropyl)-5,8-diazaspiro[3.5]nonan-5-yl)-5-methyl-1H-pyrazol-1-yl)-2-azaspiro[3.3]heptane-2-carboxylate

To a solution of tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate (Intermediate C28, 11.8 g, 18.2 mmol) in DMF (90 mL) under an argon atmosphere was added portionwise while cooling LiClO₄ (19.4 g, 182 mmol) (exothermic) followed by 2,2-dimethyloxirane (32.5 mL, 364 mmol). The reaction mixture was stirred 2 h at 70° C. The RM was cooled to RT, poured into a sat. aq. solution of NaHCO₃ and extracted with CH₂Cl₂ (×3). The combined organic extracts were washed with brine, dried (MgSO₄) and concentrated. The crude residue was purified by normal chromatography (eluent: hexane/(CH₂Cl₂/MeOH 9/1) in hexane 1/9 to 1/1) to give the title compound as a beige foam. UPLC-MS-3: Rt=1.10 min; MS m/z [M+H]⁺ 722.6/724.6.

### Step 2: 1-(5-(4-(5-chloro-6-methyl-1H-indazol-4-yl)-5-methyl-1-(2-azaspiro[3.3]heptan-6-yl)-1H-pyrazol-3-yl)-5,8-diazaspiro[3.5]nonan-8-yl)-2-methylpropan-2-ol

To a solution of tert-butyl 6-(4-(5-chloro-6-methyl-1-(tetrahydro-2H-pyran-2-yl)-1H-indazol-4-yl)-3-(8-(2-hydroxy-2-methylpropyl)-5,8-diazaspiro[3.5]nonan-5-yl)-5-methyl-1H-pyrazol-1-yl)-2-azaspiro[3.3]heptane-2-carboxylate (Step 1, 11.7 g, 16.2 mmol) in CH₂Cl₂ (70 mL) was added TFA (37.4 mL, 486 mmol). The resulting solution was stirred for 2 h at RT. The reaction mixture was concentrated and dried overnight under high vacuum to give the title compound as a trifluoroacetate salt which was used without purification in the next step. UPLC-MS-3: Rt=0.41 min; MS m/z [M+H]⁺ 538.5/540.5.

### Step 3: 1-(6-(4-(5-chloro-6-methyl-1H-indazol-4-yl)-3-(8-(2-hydroxy-2-methylpropyl)-5,8-diazaspiro[3.5]nonan-5-yl)-5-methyl-1H-pyrazol-1-yl)-2-azaspiro[3.3]heptan-2-yl)prop-2-en-1-one

To a solution of acrylic acid (1.38 mL, 20.0 mmol) and T₃P (50% in EtOAc, 11.9 mL, 20.0 mmol) in CH₂Cl₂ (38 mL) under argon atmosphere was added DIPEA (17.5 mL, 100 mmol) at 5° C. The cooling bath was removed and the resulting solution was stirred for 10 min, then was added dropwise (over 30 min) to a cooled solution (5° C.) of 1-(5-(4-(5-chloro-6-methyl-1H-indazol-4-yl)-5-methyl-1-(2-azaspiro[3.3]heptan-6-yl)-1H-pyrazol-3-yl)-5,8-diazaspiro[3.5]nonan-8-yl)-2-methylpropan-2-ol (Step 2, 10.0 mmol) in CH₂Cl₂ (38 mL). The reaction mixture was allowed to reach 15° C. and was stirred for 20 min. The RM was cooled to 5° C., THF (10 mL) and LiOH (2M, 75 mL, 150 mmol) were added. The RM was stirred for 30 min at RT, then was poured into a sat. aq. NaHCO₃ solution and extracted with CH₂Cl₂ (3×). The combined organic extracts were washed with a sat. aq. NaHCO₃ solution, dried (MgSO₄) and concentrated. The crude residue was purified by normal phase chromatography (eluent: (EtOAc/MeOH 9/1) in hexane from 10 to 100%) to give the title product. The isomers were separated by chiral SFC (C-SFC-4; mobile phase: CO₂/IPA 65/35) to give the second eluting peak which was further purified by normal phase chromatography (eluent: (CH₂Cl₂/MeOH 9/1) in hexane from 0 to 100%) to give the title compound Example 160b (white powder): ¹H NMR (600 MHz, DMSO-d₆) δ 12.9 (s, 1H), 7.56 (d, 1H), 7.44 (s, 1H), 6.31 (m, 1H), 6.10 (m, 1H), 5.67 (m, 1H), 4.75 (m, 1H), 4.34 (s, 1H), 4.29 (s, 1H), 4.06 (s, 1H), 4.00 (s, 1H), 2.82-2.65 (m, 6H), 2.51 (m, 2H, overlapping with DMSO peak), 2.48 (s, 3H), 2.37 (m, 1H), 2.25-2.11 (m, 4H), 2.06 (d, 2H), 1.99 (s, 3H), 1.81 (m, 1H), 1.62-1.48 (m, 3H), 1.06 (s, 6H); UPLC-MS-3: Rt=0.68 min; MS m/z [M+H]⁺ 592.5/594.5; C-SFC-3 (mobile phase: CO₂/[IPA+0.1% Et₃N]: 70/30): Rt=2.71 min. The other isomer Example 160a was obtained as the first eluting peak: C-SFC-3 (mobile phase: CO₂/[IPA+0.1% Et₃N]: 70/30): Rt=1.69 min.

The following examples 161a to 168 were prepared using analogous methods to Method-7 from intermediates (in Step 1) described in the intermediates synthesis section or commercially available.

When the final separation of isomers is performed by preparative HPLC using acidic conditions the purified fractions were extracted with a sat. aq. NaHCO₃ solution before lyophilization from CH₃CN/H₂O to give the title compound as a free base.

| | | Method, | |
|---|---|---|---|
| | | intermediates | |
| | | (Step 1) and | |
| | | chiral | |
| | | separation | |
| | | conditions | |
| | | used and | |
| | | order of | |
| Example | Structure | elution | Characterizing data |
| 161a/ 161b | 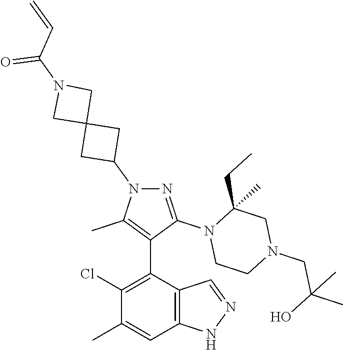 [SMILES: C=CC(=O)N1CC2(CC(n3nc(N4CCN(CC(C)(C)O)C[C@]4(C)CC)c(-c4c(Cl)c(C)cc5[nH]ncc45)c3C)C2)C1] | Using Method- 7 from Intermediate C39 and C- SFC-1 (mobile phase: [IPA + 0.1% Et₃N]/CO₂ 25:75); Example 161a = 1^{st} eluting isomer, Example 161b = 2^{nd} eluting isomer | Example 161b: ¹H NMR (600 MHz, DMSO-d₆) δ 12.96 (s, 1H), 7.57 (m, 1H), 7.44 (s, 1H), 6.30 (m, 1H), 6.10 (m, 1H), 5.67 (m, 1H), 4.72 (m, 1H), 4.33 (s, 1H), 4.29 (m, 1H), 4.04 (s, 1H), 4.00 (m, 1H), 3.94 (m, 1H), 2.99 (m, 1H), 2.81 (m, 1H), 2.68-2.76 (m, 3H), 2.65 (m, 1H), 2.47 (s, 3H), 2.06-2.32 (m, 3H), 1.95 (s, 3H), 1.94-2.04 (m, 3H), 1.84 (m, 1H), 1.67 (m, 1H), 1.03 (s, 6H), 0.92 (m, 3H), 0.64 (m, 3H). UPLC- MS-3: Rt = 0.75 min, MS m/z [M + H]⁺ 594.6/596.6; C-SFC-3 (mobile phase: [IPA + 0.1% NH₃]/CO₂ 25:75): Rt = 3.37 min. Example 161a: C-SFC-3 (mobile phase: [IPA + 0.1% NH₃]/CO₂ 25:75): Rt = 2.65 min. |
| | (S)-1-(6-(4-(5-chloro-6- | | |
| | methyl-1H-indazol-4-yl)-3- | | |
| | (2-ethyl-4-(2-hydroxy-2- | | |
| | methylpropyl)-2- | | |
| | methylpiperazin-1-yl)-5- | | |
| | methyl-1H-pyrazol-1-yl)-2- | | |
| | azaspiro[3.3]heptan-2- | | |
| | yl)prop-2-en-1-one | | |
| 162a/ 162b | 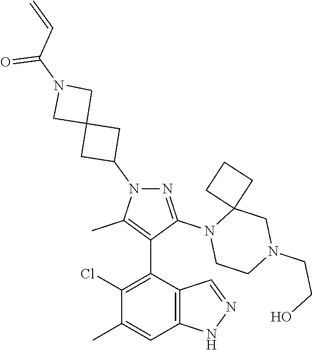 [SMILES: C=CC(=O)N1CC2(CC(n3nc(N4CCN(CCO)CC45CCC5)c(-c4c(Cl)c(C)cc5[nH]ncc45)c3C)C2)C1] | Using Method- 7 from Intermediate C28 and oxirane and C- SFC-1 (mobile phase: [IPA + 0.1% Et₃N]/CO₂ 32:68); Example 162a = 1^{st} eluting isomer, Example 162b = 2^{nd} eluting isomer | Example 162b: ¹H NMR (400 MHz, DMSO-d₆) δ 12.94 (s, 1H), 7.56 (s, 1H), 7.43 (s, 1H), 6.30 (m, 1H), 6.09 (m, 1H), 5.67 (m, 1H), 4.74 (m, 1H), 4.33 (s, 1H), 4.28 (s, 1H), 4.26 (m, 1H), 4.05 (s, 1H), 4.00 (s, 1H), 3.43 (m, 2H), 2.58-2.82 (m, 6H), 2.47 (s, 3H), 2.40 (m, 1H), 2.17-2.35 (m, 4H), 2.00-2.17 (m, 3H), 1.98 (s, 3H), 1.78 (m, 1H), 1.51-1.67 (m, 3H). UPLC-MS-2a: Rt = 0.81 min, MS m/z [M + H]⁺ 564.5/ 566.5; C-SFC-3 (mobile phase: [IPA + 0.1% NH₃]/CO₂ 35:65): Rt = 3.06 min. Example 162a: C- SFC-3 (mobile phase: [IPA + 0.1% NH₃]/CO₂ 35:65): Rt = 1.91 min. |
| | 1-(6-(4-(5-chloro-6-methyl- | | |
| | 1H-indazol-4-yl)-3-(8-(2- | | |
| | hydroxyethyl)-5,8- | | |
| | diazaspiro[3.5]nonan-5-yl)- | | |
| | 5-methyl-1H-pyrazol-1-yl)-2- | | |
| | azaspiro[3.3]heptan-2- | | |
| | yl)prop-2-en-1-one | | |
| 163a/ 163b | 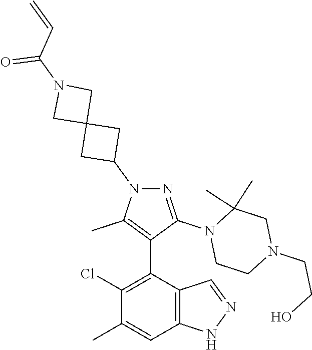 [SMILES: C=CC(=O)N1CC2(CC(n3nc(N4CCN(CCO)CC4(C)C)c(-c4c(Cl)c(C)cc5[nH]ncc45)c3C)C2)C1] | Using Method- 7 from Intermediate C38 and oxirane and C- SFC-4 (mobile phase: [IPA + 0.1% Et₃N]/CO₂ 30:70); Example 163a = 1^{st} eluting isomer, Example 163b = 2^{nd} eluting isomer | Example 163b: ¹H NMR (400 MHz, DMSO-d₆) δ 12.95 (s, 1H), 7.57 (s, 1H), 7.44 (s, 1H), 6.30 (m, 1H), 6.09 (m, 1H), 5.67 (m, 1H), 4.73 (m, 1H), 4.33 (s, 1H), 4.29 (s, 1H), 4.23 (m, 1H), 4.05 (s, 1H), 4.00 (s, 1H), 3.39 (m, 2H), 2.62-2.87 (m, 6H), 2.47 (s, 3H), 2.17 (m, 2H), 2.07 (m, 1H), 1.97 (s, 3H), 1.77-2.03 (m, 3H), 1.15 (m, 3H), 1.06 (m, 3H). UPLC-MS-2a: Rt = 0.78 min, MS m/z [M + H]⁺ 552.3/554.3; C- SFC-3 (mobile phase: 30:70 [IPA + 0.1% NH₃]/CO₂ 30:70): Rt = 2.23 min. Example 163a: C- SFC-3 (mobile phase: [PA + 0.1% NH₃]/CO₂ 30:70): Rt = 1.54 min |
| | 1-(6-(4-(5-chloro-6-methyl- | | |
| | 1H-indazol-4-yl)-3-(4-(2- | | |
| | hydroxyethyl)-2,2- | | |
| | dimethylpiperazin-1-yl)-5- | | |
| | methyl-1H-pyrazol-1-yl)-2- | | |
| | azaspiro[3.3]heptan-2- | | |
| | yl)prop-2-en-1-one | | |
| 164a/ 164b | 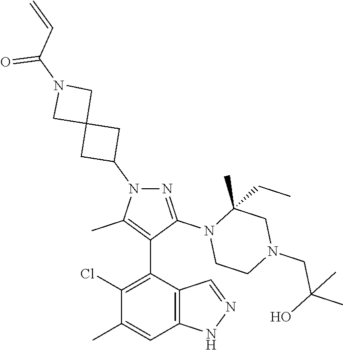 [SMILES: C=CC(=O)N1CC2(CC(n3nc(N4CCN(CC(C)(C)O)C[C@@]4(C)CC)c(-c4c(Cl)c(C)cc5[nH]ncc45)c3C)C2)C1] | Using Method- 7 from intermediate C40 and C- SFC-1 (mobile phase: CO₂/[IPA + 0.1% Et₃N] 75/25); Example 164a = 1^{st} eluting isomer, Example 164b = 2^{nd} eluting isomer | Example 164b: ¹H NMR (400 MHz, DMSO-d₆) δ 12.9 (s, 1H), 7.57 (s, 1H), 7.44 (s, 1H), 6.30 (m, 1H), 6.09 (m, 1H), 5.67 (m, 1H), 4.72 (m, 1H), 4.33 (m, 1H), 4.29 (s, 1H), 4.04 (m, 1H), 4.00 (m, 1H), 3.94 (s, 1H), 2.96-2.87 (m, 1H), 2.87-2.77 (m, 1H), 2.76- 2.65 (m, 4H), 2.47 (s, 3H), 2.33- 2.16 (m, 2H), 2.06-1.94 (m, 4H), 1.95 (s, 3H), 1.83-1.71 (m, 1H), 1.71-1.57 (m, 1H), 1.07-1.00 (m, 9H), 0.63-0.56 (m, 3H). UPLC- MS-2e: Rt = 3.55 min, MS m/z [M + H]⁺ 594.4/596.3; C-SFC-3 (mobile phase: CO₂/[IPA + 0.025% NH₃] 75/25): Rt = 3.22 min. Example 164a: C-SFC-3 (mobile phase: CO₂/[IPA + 0.025% NH₃] 75/25): Rt = 2.21 min. |
| | (R)-1-(6-(4-(5-chloro-6- | | |
| | methyl-1H-indazol-4-yl)-3- | | |
| | (2-ethyl-4-(2-hydroxy-2- | | |
| | methylpropyl)-2- | | |
| | methylpiperazin-1-yl)-5- | | |
| | methyl-1H-pyrazol-1-yl)-2- | | |
| | azaspiro[3.3]heptan-2- | | |
| | yl)prop-2-en-1-one | | |
| 165a/ 165b | 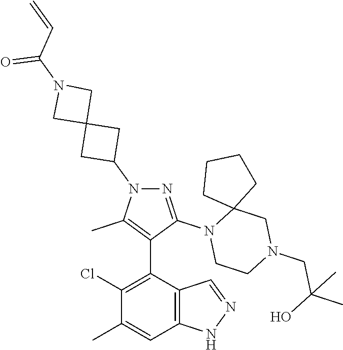 [SMILES: C=CC(=O)N1CC2(CC(n3nc(N4CCN(CC(C)(C)O)CC45CCCC5)c(-c4c(Cl)c(C)cc5[nH]ncc45)c3C)C2)C1] | Using Method- 7 from intermediate C31 and C- SFC-1 (mobile phase: CO₂/[IPA + 0.1% Et₃N] 72/28); Example 165a = 1^{st} eluting isomer, Example 165b = 2^{nd} eluting isomer | Example 165b: ¹H NMR (400 MHz, DMSO-d₆) δ 12.9 (s, 1H), 7.54 (s, 1H), 7.44 (s, 1H), 6.31 (m, 1H), 6.09 (m, 1H), 5.67 (m, 1H), 4.74 (m, 1H), 4.33 (s, 1H), 4.26 (m, 1H), 4.05 (s, 1H), 3.97 (s, 1H), 3.95 (s, 1H), 2.81-2.65 (m, 6H), 2.48 (s, 3H), 2.28-2.17 (m, 2H), 2.15-2.09 (m, 1H), 2.06- 1.94 (m, 5H), 1.97 (s, 3H), 1.70- 1.31 (m, 6H), 1.04-1.01 (m, 6H). UPLC-MS-2e: Rt = 3.58 min, MS m/z [M + H]⁺ 606.4/608.4; C- SFC-3 (mobile phase: CO₂/[IPA + 0.025% NH₃] 72/28): Rt = 3.13 min. Example 165a: C-SFC-3 (mobile phase: CO₂/[IPA + 0.025% NH₃] 75/25): Rt = 1.99 min. |
| | 1-(6-(4-(5-chloro-6-methyl- | | |
| | 1H-indazol-4-yl)-3-(9-(2- | | |
| | hydroxy-2-methylpropyl)- | | |
| | 6,9-diazaspiro[4.5]decan-6- | | |
| | yl)-5-methyl-1H-pyrazol-1- | | |
| | yl)-2-azaspiro[3.3]heptan-2- | | |
| | yl)prop-2-en-1-one | | |
| 166a/ 166b | 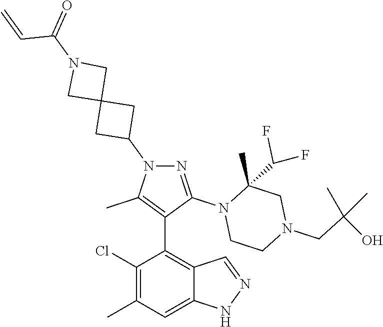 [SMILES: C=CC(=O)N1CC2(CC(n3nc(N4CCN(CC(C)(C)O)C[C@@]4(C)C(F)F)c(-c4c(Cl)c(C)cc5[nH]ncc45)c3C)C2)C1] | Using Method- 7 from intermediate Intermediate C43 and C- SFC-35 (mobile phase: CO₂/[IPA + 0.1% Et₃N] 70/30); Example 166a = 1^{st} eluting isomer, Example 166b = 2^{nd} eluting isomer | Example 166b: ¹H NMR (400 MHz, DMSO-d₆) δ 13.02 (s, 1H), 7.58 (s, 1H), 7.47 (s, 1H), 6.72- 6.37 (m, 1H), 6.35-6.23 (m, 1H), 6.16-6.04 (m, 1H), 5.73-5.63 (m, 1H), 4.84-4.69 (m, 1H), 4.34 (s, 1H), 4.29 (s, 1H), 4.05 (s, 1H), 4.00 (s, 1H), 3.98 (s, 1H), 3.22- 3.09 (m, 1H), 2.85-2.63 (m, 6H), 2.48 (s, 3H), 2.42-2.27 (m, 1H), 2.17-2.07 (m, 1H), 2.03-1.94 (m, 1H), 2.02 (s, 2H), 1.96 (s, 3H), 1.05-0.97 (m, 9H). UPLC-MS-4: Rt = 0.82 min, MS m/z [M + H]⁺ 616.5/618.5; C-SFC-3 (mobile phase: CO₂/[IPA + 0.025% NH₃] 70/30): Rt = 1.67 min. Example 166a: C-SFC-3 (mobile phase: CO₂/[IPA + 0.025% NH₃] 70/30): Rt = 1.23 min. |
| | (S)-1-(6-(4-(5-chloro-6- | | |
| | methyl-1H-indazol-4-yl)-3- | | |
| | (2-(difluoromethyl)-4-(2- | | |
| | hydroxy-2-methylpropyl)-2- | | |
| | methylpiperazin-1-yl)-5- | | |
| | methyl-1H-pyrazol-1-yl)-2- | | |
| | azaspiro[3.3]heptan-2- | | |
| | yl)prop-2-en-1-one | | |
| 167a/ 167b | 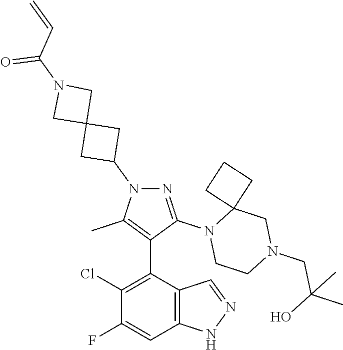 [SMILES: C=CC(=O)N1CC2(CC(n3nc(N4CCN(CC(C)(C)O)CC45CCC5)c(-c4c(Cl)c(F)cc5[nH]ncc45)c3C)C2)C1] | Using Method- 7 from intermediate C32 and C- SFC-4 (mobile phase: CO₂/IPA 70/30); Example 167a = 1^{st} eluting isomer, Example 167b = 2^{nd} eluting isomer | Example 167b: ¹H NMR (400 MHz, DMSO-d₆) δ 13.1 (s, 1H), 7.68 (s, 1H), 7.49 (d, 1H), 6.31 (m, 1H), 6.09 (m, 1H), 5.67 (m, 1H), 4.74 (m, 1H), 4.34 (s, 1H), 4.29 (m, 1H), 4.06 (s, 1H), 3.98 (s, 1H), 2.78-2.65 (m, 6H), 2.60- 2.52 (m, 1H), 2.35-2.24 (m, 2H), 2.23-2.12 (m, 2H), 2.10-2.05 (m, 1H), 2.08 (d, 1H), 2.05 (d, 1H), 2.03 (s, 3H), 1.78-1.71 (m, 1H), 1.57-1.44 (m, 3H), 1.06-1.02 (m, 6H). UPLC-MS-2e: Rt = 3.33 min, MS m/z [M + H]⁺ 596.4/ 598.4; C-SFC-3 (mobile phase: CO₂/IPA 70/30): Rt = 2.01 min. Example 167a: C-SFC-3 (mobile phase: CO₂/IPA 70/30): Rt = 1.30 min. |
| | 1-(6-(4-(5-chloro-6-fluoro- | | |
| | 1H-indazol-4-yl)-3-(8-(2- | | |
| | hydroxy-2-methylpropyl)- | | |
| | 5,8-diazaspiro[3.5]nonan-5- | | |
| | yl)-5-methyl-1H-pyrazol-1- | | |
| | yl)-2-azaspiro[3.3]heptan-2- | | |
| | yl)prop-2-en-1-one | | |
| 168 | 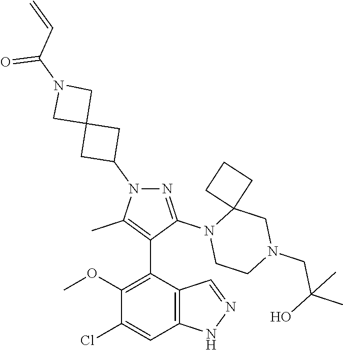 [SMILES: C=CC(=O)N1CC2(CC(n3nc(N4CCN(CC(C)(C)O)CC45CCC5)c(-c4c(OC)c(Cl)cc5[nH]ncc45)c3C)C2)C1] | Using Method- 7 and Intermediate C37 | Example 168: UPLC-MS-4: Rt = 0.67 min; MS m/z [M + H]⁺ 608.4/ 610.4. |
| | 1-(6-(4-(6-chloro-5-methoxy- | | |
| | 1H-indazol-4-yl)-3-(8-(2- | | |
| | hydroxy-2-methylpropyl)- | | |
| | 5,8-diazaspiro[3.5]nonan-5- | | |
| | yl)-5-methyl-1H-pyrazol-1- | | |
| | yl)-2-azaspiro[3.3]heptan-2- | | |
| | yl)prop-2-en-1-one | | |

### Method-8 for the Synthesis of Examples 169a and 169b: (R)-1-(6-(3-(8-((1,4-Dioxan-2-yl)methyl)-5,8-diazaspiro[3.5]nonan-5-yl)-4-(5-chloro-6-methyl-1H-indazol-4-yl)-5-methyl-1H-pyrazol-1-yl)-2-azaspiro[3.3]heptan-2-yl)prop-2-en-1-one

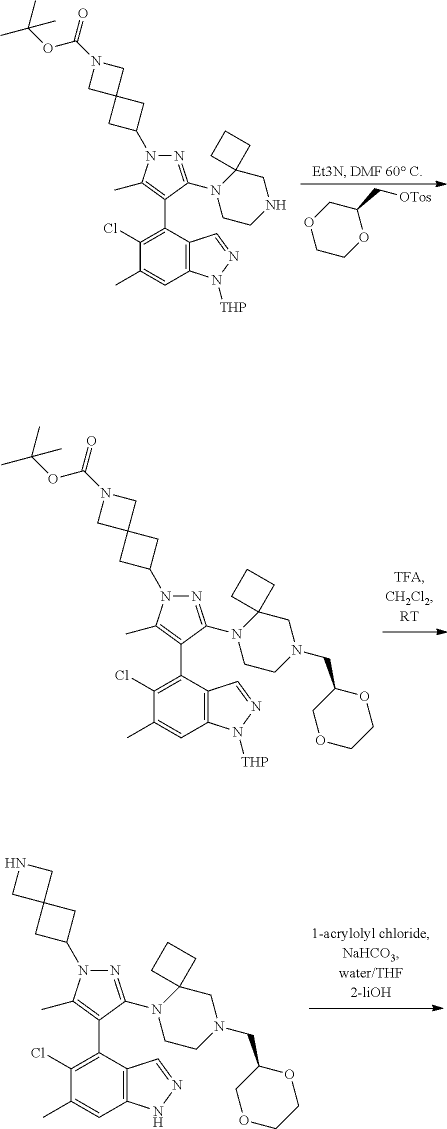

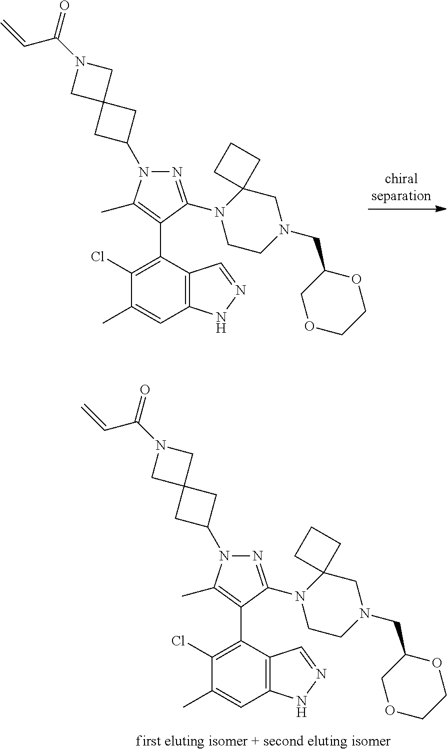 [SMILES: C=CC(=O)N1CC2(CC(n3nc(N4CCN(C[C@@H]5COCCO5)CC45CCC5)c(-c4c(Cl)c(C)cc5[nH]ncc45)c3C)C2)C1 ; C=CC(=O)N1CC2(CC(n3nc(N4CCN(C[C@@H]5COCCO5)CC45CCC5)c(-c4c(Cl)c(C)cc5[nH]ncc45)c3C)C2)C1]

### Step 1: Tert-butyl 6-(3-(8-(((R)-1,4-dioxan-2-yl)methyl)-5,8-diazaspiro[3.5]nonan-5-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate

To tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate (intermediate C28, 0.51 mmol, 350 mg) in DMF (3 mL) was added under argon a solution of (S)-(1,4-dioxan-2-yl)methyl 4-methylbenzenesulfonate (Intermediate B1, 184 mg, 0.61 mmol) and triethylamine (0.21 mL, 1.52 mmol). The reaction mixture was stirred at 60° C. for 11 h. The reaction mixture was diluted with a sat. aq. NaHCO₃ solution, extracted with EtOAc and the combined organic extracts were washed with brine, dried (Na₂SO₄), filtered and evaporated. The crude residue was purified by normal phase chromatography (eluent: EtOAc in n-heptane 0 to 100%). UPLC-MS-3: Rt=1.10 min; MS m/z [M+H]⁺ 750.5/752.5.

### Step 2: (R)-8-((1,4-Dioxan-2-yl)methyl)-5-(4-(5-chloro-6-methyl-1H-indazol-4-yl)-5-methyl-1-(2-azaspiro[3.3]heptan-6-yl)-1H-pyrazol-3-yl)-5,8-diazaspiro[3.5]nonane

To a solution of tert-butyl 6-(3-(8-(((R)-1,4-dioxan-2-yl)methyl)-5,8-diazaspiro[3.5]nonan-5-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Step 1, 320 mg, 0.39 mmol) in CH₂Cl₂ (8 mL) was added under argon TFA (0.60 mL, 7.79 mmol) and the reaction mixture was stirred at RT for 2 days. TFA (0.30 mL, 3.89 mmol) was added again and the reaction mixture was stirred for another 24 h to complete the reaction. The reaction mixture was evaporated to dryness to give the title compound as a trifluoroacetate salt which was used without purification in the next step. UPLC-MS-3: Rt=0.45 min; MS m/z [M+H]⁺ 566.5/568.5.

### Step 3: (R)-1-(6-(3-(8-((1,4-Dioxan-2-yl)methyl)-5,8-diazaspiro[3.5]nonan-5-yl)-4-(5-chloro-6-methyl-1H-indazol-4-yl)-5-methyl-1H-pyrazol-1-yl)-2-azaspiro[3.3]heptan-2-yl)prop-2-en-1-one

To a solution of (R)-8-((1,4-dioxan-2-yl)methyl)-5-(4-(5-chloro-6-methyl-1H-indazol-4-yl)-5-methyl-1-(2-azaspiro[3.3]heptan-6-yl)-1H-pyrazol-3-yl)-5,8-diazaspiro[3.5]nonane (Step 2, 0.40 mmol) in THF (8 mL) under argon were added water (0.21 mL) and NaHCO₃ (670 mg, 7.97 mmol) followed by acryloyl chloride (0.04 mL, 0.48 mmol). The reaction mixture was stirred at RT for 30 min. Acryloyl chloride (0.01 mL, 0.12 mmol) was added again and the RM was stirred for 30 min to complete the reaction. Then, LiOH (2 M, 2 mL, 4 mmol) was added and the RM was stirred at RT for 30 min until disappearance (UPLC) of the side product resulting from reaction of the acryloyl chloride with the indazole NH. The RM was diluted with water, extracted with EtOAc (×2) and the combined organic extracts were washed with brine, dried (Na₂SO₄), filtered and evaporated. The crude residue was purified by preparative HPLC (RP-HPLC-1; mobile phase: A: water+0.1% TFA, B: acetonitrile; gradient: 10 to 95% B in 25 min), the purified fractions were neutralized by addition of a sat. aq. NaHCO₃ solution and extracted with EtOAc (×2) to give after evaporation under reduced pressure the title compound. The isomers were separated by chiral SFC (C-SFC-4; mobile phase: CO₂/[IPA+0.1% Et₃N] 70/30) to give the title compound as the second eluting peak Example 169b (white powder): ¹H NMR (600 MHz, DMSO-d₆) δ 12.9 (s, 1H), 7.56 (d, 1H), 7.43 (s, 1H), 6.31 (m, 1H), 6.10 (m, 1H), 5.67 (m, 1H), 4.74 (m, 1H), 4.33 (s, 1H), 4.28 (m, 1H), 4.05 (s, 1H), 3.99 (m, 1H), 3.70-3.63 (m, 2H), 3.61-3.56 (m, 2H), 3.51 (td, 1H), 3.39 (td, 1H), 3.16 (m, 1H), 2.80-2.62 (m, 6H), 2.47 (s, 3H), 2.34 (m, 1H), 2.26-2.07 (m, 6H), 2.02 (m, 1H), 1.98 (s, 3H), 1.76 (m, 1H), 1.62-1.52 (m, 3H); UPLC-MS-3: Rt=0.68 min; MS m/z [M+H]⁺ 620.4/622.5; C-SFC-3 (mobile phase: CO₂/[IPA+0.1% Et₃N]: 70/30): Rt=2.51 min. The other isomer Example 169a was obtained as the first eluting peak: C-SFC-3 (mobile phase: CO₂/[IPA+0.1% Et₃N]: 70/30): Rt=1.34 min.

Method-8a: similar to Method-8 except that Step 1 was performed in CH₃CN at 80° C. instead of DMF at 60° C.

Method-8b: similar to Method-8 except that Step 3 was performed using Et₃N, acrylic acid or a substituted acrylic acid and T₃P in CH₂Cl₂ as described in Step 3 in Method-7.

Method-8c: similar to Method-8 except that NaI (1 eq.) was added in Step 1.

Method-8d: similar to Method-8 except that Step 1 was performed in CH₃CN at 80° instead of DMF at 60° C. and that NaI (1 eq.) was added.

The following examples 170a to 189b were prepared using analogous methods to Method-8 from intermediates (in Step 1, 2 or 3) described in the intermediates synthesis section or commercially available.

When the final separation of isomers is performed by preparative HPLC using acidic conditions the purified fractions were extracted with a sat. aq. NaHCO₃ solution before lyophilization from a mixture of CH₃CN and H₂O to give the title compound as a free base.

| | | Method, intermediates | |
|---|---|---|---|
| | | (Step 1, 2 or 3) and | |
| | | chiral separation | |
| | | conditions used and | |
| Example | Structure | order of elution | Characterizing data |
| 170a/ 170b | 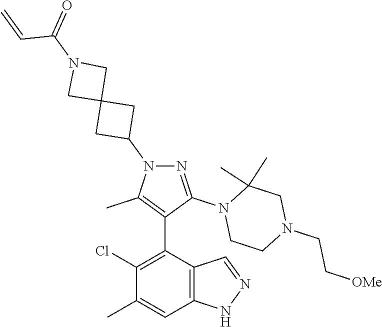 [SMILES: C=CC(=O)N1CC2(CC(n3nc(N4CCN(CCOC)CC4(C)C)c(-c4c(Cl)c(C)cc5[nH]ncc45)c3C)C2)C1] 1-(6-(4-(5-chloro-6-methyl- 1H-indazol-4-yl)-3-(4-(2- methoxyethyl)-2,2- dimethylpiperazin-1-yl)-5- methyl-1H-pyrazol-1-yl)-2- | Using Method-8ab from Intermediate C38 and 1- bromo-2- methoxyethane (Step 1) and C-SFC-2 (mobile phase: CO₂/[IPA + 0.1% Et₃N] 75/25); Example 170a = 1^{st} eluting isomer, Example 170b = 2^{nd} eluting isomer. | Example 170b: ¹H NMR (600 MHz, DMSO-d₆) δ 12.9 (s, 1H), 7.57 (s, 1H), 7.44 (s, 1H), 6.31 (m, 1H), 6.10 (m, 1H), 5.67 (m, 1H), 4.73 (m, 1H), 4.33 (s, 1H), 4.29 (s, 1H), 4.05 (s, 1H), 4.00 (s, 1H), 3.36 (m, 2H), 3.17 (s, 3H), 2.82-2.76 (m, 2H), 2.74-2.66 (m, 4H), 2.48 (s, 3H), 2.29-2.21 (m, 2H), 2.10-2-06 (m, 1H), 2.02-1.90 (m, 6H), 1.15 (m, 3H), 1.05 (m, 3H). UPLC-MS-2e: Rt = 3.24 min; MS m/z [M + H]⁺ 566.3/568.3; C-SFC-3 (mobile phase: CO₂/[IPA + 0.1% Et₃N] 75/25): Rt = 2.86 min, Example 170a: C-SFC-3 (mobile phase: CO₂/[IPA + 0.1% Et₃N] 75/25): Rt = 2.23 min. |
| | azaspiro[3.3]heptan-2- | | |
| | yl)prop-2-en-1-one | | |
| 171a/ 171b | 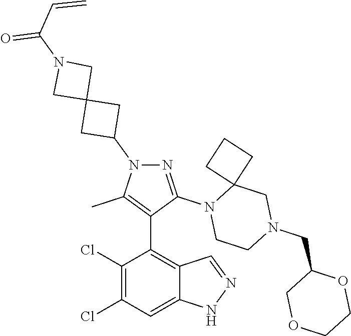 [SMILES: C=CC(=O)N1CC2(CC(n3nc(N4CCN(C[C@@H]5COCCO5)CC45CCC5)c(-c4c(Cl)c(Cl)cc5[nH]ncc45)c3C)C2)C1] (R)-1-(6-(3-(8-((1,4-dioxan- 2-yl)methyl)-5,8- diazaspiro[3.5]nonan-5-yl)- 4-(5,6-dichloro-1H-indazol- | | Example 171b: ¹H NMR (400 MHz, DMSO-d₆) δ 13.2 (s, 1H), 7.78 (m, 1H), 7.67 (s, 1H), 6.30 (m, 1H), 6.09 (m, 1H), 5.67 (m, 1H), 4.76 (m, 1H), 4.34 (s, 1H), 4.28 (m, 1H), 4.05 (s, 1H), 4.00 (m, 1H), 3.71-3.47 (m, 5H), 3.43-3.35 (m, 1H), 3.16 (m, 1H), 2.79- 2.64 (m, 6H), 2.39-2.24 (m, 2H), 2.24-2.02 (m, 6H), 2.01 (s, 3H), 1.79- 1.68 (m, 1H), 1.62-1.47 (m, 3H). UPLC-MS-2e: Rt = 3.51 min; MS m/z [M + H]⁺ 640.3/642.3; C- SFC-3 (mobile phase: CO₂/[IPA + 0.025% NH₃] 70/30): Rt = 2.45 min, Example 171a: C-SFC-3 (mobile phase: CO₂/[IPA + 0.025% NH₃] 70/30): Rt = 1.38 min. |
| | 4-yl)-5-methyl-1H-pyrazol- | | |
| | 1-yl)-2-azaspiro[3.3]heptan- | | |
| | 2-yl)prop-2-en-1-one | | |
| 172a/ 172b | 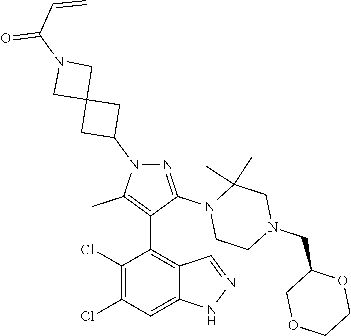 [SMILES: C=CC(=O)N1CC2(CC(n3nc(N4CCN(C[C@@H]5COCCO5)CC4(C)C)c(-c4c(Cl)c(Cl)cc5[nH]ncc45)c3C)C2)C1] (R)-1-(6-(3-(4-((1,4-dioxan- 2-yl)methyl)-2,2- dimethylpiperazin-1-yl)-4- | Using Method-8b from Intermediate C34 and intermediate B1 (Step 1) and C-SFC-2 (mobile phase: CO₂/[IPA + 0.1% Et₃N] 70/30); Example 172a = 1^{st} eluting isomer, Example 172b = 2^{nd} eluting isomer. | Example 172b: ¹H NMR (400 MHz, DMSO-d₆) δ 13.2 (s, 1H), 7.79 (s, 1H), 7.67 (s, 1H), 6.30 (m, 1H), 6.09 (m, 1H), 5.67 (m, 1H), 4.74 (m, 1H), 4.33 (s, 1H), 4.29 (s, 1H), 4.05 (s, 1H), 4.00 (s, 1H), 3.72-3.45 (m, 5H), 3.42- 3.34 (m, 1H), 3.18-3.11 (m, 1H), 2.89-2.78 (m, 2H), 2.77-2.68 (m, 4H), 2.14-1.87 (m, 9H), 1.12- 1.06 (m, 3H), 1.06-0.96 (m, 3H). UPLC-MS-2e: Rt = 3.44 min; MS m/z [M + H]⁺ 628.4/630.4; C- SFC-3 (mobile phase: CO₂/[IPA + 0.025% NH₃] 73/27): Rt = 2.74 min, Example 172a: C-SFC-3 (mobile phase: CO₂/[IPA + 0.025% NH₃] 73/27): Rt = 1.78 min. |
| | (5,6-dichloro-1H-indazol-4- | | |
| | yl)-5-methyl-1H-pyrazol-1- | | |
| | yl)-2-azaspiro[3.3]heptan-2- | | |
| | yl)prop-2-en-1-one | | |
| 173a/ 173b | 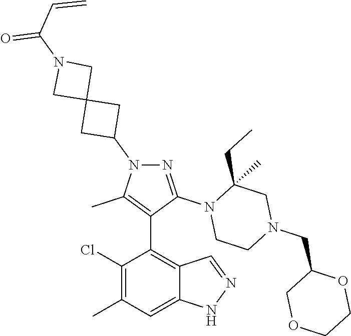 [SMILES: C=CC(=O)N1CC2(CC(n3nc(N4CCN(C[C@@H]5COCCO5)C[C@]4(C)CC)c(-c4c(Cl)c(C)cc5[nH]ncc45)c3C)C2)C1] 1-(6-(3-((S)-4-(((R)-1,4- dioxan-2-yl)methyl)-2-ethyl- 2-methylpiperazin-1-yl)-4-(5-chloro-6-methyl-1H- indazol-4-yl)-5-methyl-1H-pyrazol-1-yl)-2- azaspiro[3.3]heptan-2-yl)prop-2-en-1-one | Using Method Method- 8abc from Intermediate C39 and intermediate B1 (Step 1) and C-SFC- 2 (mobile phase: CO₂/[IPA + 0.1% Et₃N] 72/28); Example 173a = 1^{st} eluting isomer, Example 173b = 2^{nd} eluting isomer. | Example 173b: ¹H NMR (400 MHz, DMSO-d₆) δ 13.0 (s, 1H), 7.58 (s, 1H), 7.45 (s, 1H), 6.31 (m, 1H), 6.10 (m, 1H), 5.67 (m, 1H), 4.73 (m, 1H), 4.33 (s, 1H), 4.29 (m, 1H), 4.05 (s, 1H), 4.01 (m, 1H), 3.70-3.46 (m, 5H), 3.42-3.36 (m, 1H), 3.16 (t, 1H), 3.02-2.92 (m, 1H), 2.86-2.62 (m, 5H), 2.48 (s, 3H), 2.17- 2.02 (m, 5H), 1.96 (s, 3H), 1.96-1.88 (m, 1H), 1.85-1.75 (m, 1H), 1.71- 1.61 (m, 1H), 0.96-0.89 (m, 3H), 0.68-0.62 (m, 3H). UPLC-MS-2e: Rt = 3.44 min; MS m/z [M + H]⁺ 622.5/624.4; C-SFC-3 (mobile phase: CO₂/[IPA + 0.025% NH₃ v/v] 70/30): Rt = 1.75 min, Example 173a: C-SFC-3 (mobile phase: |
| 174a/ 174b | 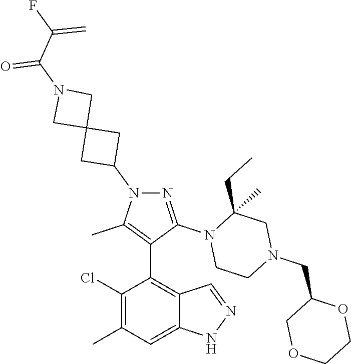 [SMILES: C=C(F)C(=O)N1CC2(CC(n3nc(N4CCN(C[C@@H]5COCCO5)C[C@]4(C)CC)c(-c4c(Cl)c(C)cc5[nH]ncc45)c3C)C2)C1] 1-(6-(3-((S)-4-(((R)-1,4-dioxan-2-yl)methyl)-2-ethyl- 2-methylpiperazin-1-yl)-4-(5-chloro-6-methyl-1H- indazol-4-yl)-5-methyl-1H-pyrazol-1-yl)-2- azaspiro[3.3]heptan-2-yl)-2- fluoroprop-2-en-1-one | Using Method-8abc from Intermediate C39 and intermediate B1 (Step 1) and 2- fluoropropenoic acid (Step 3) and C-SFC-34 (mobile phase: CO₂/[IPA + 0.1% Et₃N] 70/30); Example 174a = 1^{st} eluting isomer, Example 174b = 2^{nd} eluting isomer. | CO₂/[IPA + 0.025% NH₃] 70/30): Rt = 1.18 min. Example 174b: ¹H NMR (400 MHz, DMSO-d₆) ? 13.0 (s, 1H), 7.57 (s, 1H), 7.44 (s, 1H), 5.53-5.37 (m, 1H), 5.28 (m, 1H), 4.71 (m, 1H), 4.50-4.43 (m, 2H), 4.11-4.04 (m, 2H), 3.70-3.46 (m, 5H), 3.42-3.35 (m, 1H), 3.15 (t, 1H), 3.00-2.91 (m, 1H), 2.84-2.61 (m, 5H), 2.47 (s, 3H), 2.16-2.01 (m, 5H), 1.98-1.87 (m, 4H), 1.83-1.76 (m, 1H), 1.71- 1.62 (m, 1H), 0.96-0.89 (m, 3H), 0.68-0.61 (m, 3H). UPLC-MS-2e: Rt = 3.78 min; MS m/z [M + H]⁺ 640.5/642.4; C-SFC-3 (mobile phase: CO₂/[IPA + 0.025% NH₃] 75/25): Rt = 2.22 min, Example 174a: C-SFC-3 (mobile phase: CO₂/[IPA + 0.025% NH₃ v/v] 75/25): Rt = 1.45 min. |
| 175a/ 175b | 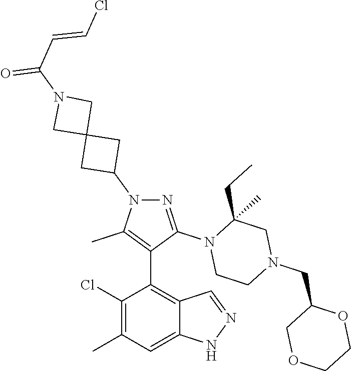 [SMILES: CC[C@@]1(C)CN(C[C@@H]2COCCO2)CCN1c1nn(C2CC3(C2)CN(C(=O)/C=C/Cl)C3)c(C)c1-c1c(Cl)c(C)cc2[nH]ncc12] (E)-1-(6-(3-((S)-4-(((R)-1,4- dioxan-2-yl)methyl)-2-ethyl- 2-methylpiperazin-1-yl)-4- | Using Method-8abc from Intermediate C39 and intermediate B1 (Step 1) and trans-3- chloroacrylic acid and C- SFC-2 (mobile phase: CO₂/[IPA + 0.1% Et₃N] 70/30); Example 175a = 1^{st} eluting isomer, Example 175b = 2^{nd} eluting isomer. | Example 175b: ¹H NMR (400 MHz, DMSO-d₆) δ 12.9 (s, 1H), 7.58 (s, 1H), 7.45 (s, 1H), 7.25 (d, 1H), 6.52 (m, 1H), 4.72 (m, 1H), 4.34 (s, 1H), 4.29 (s, 1H), 4.04(s, 1H), 3.99 (s, 1H), 3.70-3.46 (m, 5H), 3.42-3.35 (m, 1H), 3.15 (t, 1H), 3.00-2.91 (m, 1H), 2.84-2.59 (m, 5H), 2.47 (s, 3H), 2.17-2.01 (m, 5H), 1.98-1.87 (m, 1H), 1.95 (s, 3H), 1.85-1.74 (m, 1H), 1.70-1.60 (m, 1H), 0.96-0.88 (m, 3H), 0.67-0.62 (m, 3H). UPLC- MS-2e: Rt = 3.98 min; MS m/z [M + H]⁺ 656.1/658.0; C-SFC-3 (mobile phase: CO₂/[IPA + 0.1% Et₃N] 70/30): Rt = 2.21 min, Example 175a: C-SFC-3 (mobile phase: CO₂/[IPA + 0.025% NH₃ v/v] 75/25): Rt = 1.51 min. |
| | (5-chloro-6-methyl-1H- | | |
| | indazol-4-yl)-5-methyl-1H- | | |
| | pyrazol-1-yl)-2- | | |
| | azaspiro[3.3]heptan-2-yl)-3- | | |
| | chloroprop-2-en-1-one | | |
| 176 | 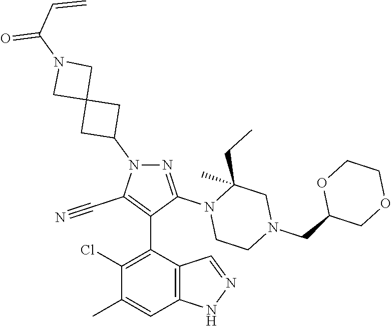 [SMILES: C=CC(=O)N1CC2(CC(n3nc(N4CCN(C[C@@H]5COCCO5)C[C@]4(C)CC)c(-c4c(Cl)c(C)cc5[nH]ncc45)c3C#N)C2)C1] | Using Method-8d from Intermediate C69 and intermediate B1 (Step 1) | Example 176: UPLC- MS-4: Rt = 0.69 min; MS m/z [M + H]⁺ 633.3/635.3. |
| | 3-((S)-4-(((R)-1,4-dioxan-2- | | |
| | yl)methyl)-2-ethyl-2- | | |
| | methylpiperazin-1-yl)-1-(2- | | |
| | acryloyl-2- | | |
| | azaspiro[3.3]heptan-6-yl)-4- | | |
| | (5-chloro-6-methyl-1H- | | |
| | indazol-4-yl)-1H-pyrazole-5- | | |
| | carbonitrile | | |
| 177a/ 177b | 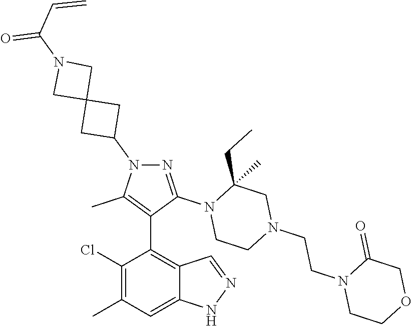 [SMILES: C=CC(=O)N1CC2(CC(n3nc(N4CCN(CCN5CCOCC5=O)C[C@]4(C)CC)c(-c4c(Cl)c(C)cc5[nH]ncc45)c3C)C2)C1] (S)-4-(2-(4-(1-(2-acryloyl-2-azaspiro[3.3]heptan-6-yl)-4- (5-chloro-6-methyl-1H-indazol-4-yl)-5-methyl-1H- pyrazol-3-yl)-3-ethyl-3-methylpiperazin-1- yl)ethyl)morpholin-3-one | Using Method-8ab from Intermediate C39 and 4- (2-bromoethyl)morpholin- 3-one (Step 1) and C- HPLC-21 (mobile phase: n- heptane/EtOH/MeOH 50/25/25 + 0.05% Et₃N); Example 177a = 1^{st} eluting isomer, Example 177b = 2^{nd} eluting isomer. | Example 177a: ¹H NMR (400 MHz, DMSO-d₆) δ 13.0 (s, 1H), 7.58 (s, 1H), 7.45 (s, 1H), 6.30 (m, 1H), 6.09 (m, 1H), 5.67 (m, 1H), 4.72 (m, 1H), 4.33 (s, 1H), 4.29 (m, 1H), 4.04 (s, 1H), 4.02- 3.95 (m, 3H), 3.79-3.74 (m, 2H), 3.39-3.33 (m, 4H), 3.02-2.91 (m, 1H), 2.86-2.79 (m, 1H), 2.76- 2.68 (m, 3H), 2.68-2.62 (m, 1H), 2.47 (s, 3H), 2.26 (m, 2H), 2.16-1.86 (m, 7H), 1.80-1.72 (m, 1H), 1.64 (m, 1H), 0.96- 0.87 (m, 3H), 0.63 (m, 3H). UPLC-MS-2e: Rt = 3.22 min; MS m/z [M + H]⁺ 649.4/651.4; C-HPLC- 22 (mobile phase: [n- heptane + 0.05% DEA]/ [EtOH/MeOH (50/50) + 0.05% DEA] |
| | | | 50/50): Rt = 5.33 min, |
| | | | Example 177b: C-HPLC- |
| | | | 22 (mobile phase: [n- |
| | | | heptane + 0.05% DEA]/ |
| | | | [EtOH/MeOH |
| | | | (50/50) + 0.05% DEA] |
| | | | 50/50): Rt = 7.45 min. |
| 178a/ 178b | 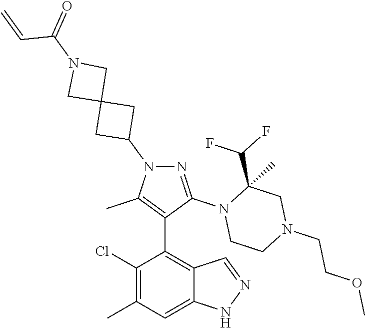 [SMILES: C=CC(=O)N1CC2(CC(n3nc(N4CCN(CCOC)C[C@]4(C)C(F)F)c(-c4c(Cl)c(C)cc5[nH]ncc45)c3C)C2)C1] (R)-1-(6-(4-(5-chloro-6-methyl-1H-indazol-4-yl)-3- (2-(difluoromethyl)-4-(2-methoxyethyl)-2- methylpiperazin-1-yl)-5-methyl-1H-pyrazol-1-yl)-2- azaspiro[3.3]heptan-2-yl)prop-2-en-1-one | Using Method-8ab from Intermediate C42 and 1- bromo-2- methoxyethane (Step 1) and C-HPLC-3 (mobile phase: n- heptane/CH₂Cl₂/EtOH 80/15/5 + 0.05% Et₃N); Example 178a = 1^{st} eluting isomer, Example 178b = 2^{nd} eluting isomer. | Example 178b ¹H NMR (400 MHz, DMSO-d₆) δ 12.99 (s, 1H), 7.64 (s, 1H), 7.47 (s, 1H), 6.90- 6.50 (m, 1H), 6.38-6.22 (m, 1H), 6.16-6.03 (m, 1H), 5.75-5.60 (m, 1H), 4.85-4.64 (m, 1H), 4.33 (s, 1H), 4.29 (s, 1H), 4.05 (s, 1H), 4.00 (s, 1H), 3.36-3.32 (m, 2H), 3.17 (s, 3H), 2.90-2.83 (m, 2H), 2.76-2.65 (m, 4H), 2.48 (s, 3H), 2.39-2.11 (m, 5H), 1.97 (s, 3H), 1.87-1.75 (m, 1H), 1.19 (s, 3H); UPLC-MS-4: Rt = 0.74 min; MS m/z [M + H]⁺ 602.7/604.1; C-HPLC-2 (mobile phase: n- heptane/CH₂Cl₂/EtOH 80/15/5 + 0.1% DEA): Rt = 20.1 min, Example 178a: C-HPLC-2 (mobile phase: n- |
| | | | heptane/CH₂Cl₂/EtOH |
| | | | 80/15/5 + 0.1% DEA): |
| | | | Rt = 14.7 min. |
| 179a/ 179b | 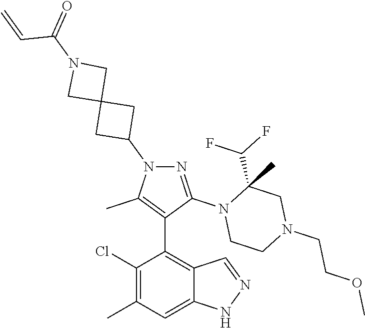 [SMILES: C=CC(=O)N1CC2(CC(n3nc(N4CCN(CCOC)C[C@@]4(C)C(F)F)c(-c4c(Cl)c(C)cc5[nH]ncc45)c3C)C2)C1] (S)-1-(6-(4-(5-chloro-6- methyl-1H-indazol-4-yl)-3- (2-(difluoromethyl)-4-(2- methoxyethyl)-2- methylpiperazin-1-yl)-5- methyl-1H-pyrazol-1-yl)-2- azaspiro[3.3]heptan-2- yl)prop-2-en-1-one | Using Method-8ab from Intermediate C43 and 1- bromo-2- methoxyethane (Step 1) and C-SFC-4 (mobile phase: CO₂/[IPA + 0.1% Et₃N] 78/22); Example 179a = 1^{st} eluting isomer, Example 179b = 2^{nd} eluting isomer. | Example 179b: ¹H NMR (600 MHz, DMSO-d₆) δ 13.04 (s, 1H), 7.59 (s, 1H), 7.48 (s, 1H), 6.61- 6.36 (m, 1H), 6.36-6.26 (m, 1H), 6.15-6.06 (m, 1H), 5.71-5.62 (m, 1H), 4.814.69 (m, 1H), 4.34 (s, 1H), 4.30 (s, 1H), 4.06 (s, 1H), 4.01 (s, 1H), 3.37- 3.33 (m, 2H), 3.19 (s, 3H), 3.14-3.06 (m, 1H), 2.83-2.75 (m, 1H), 2.75- 2.65 (m, 5H), 2.49 (s, 3H), 2.34-2.26 (m, 3H), 2.02-1.97 (m, 1H), 1.97 (s, 3H), 1.94-1.84 (m, 1H), 1.09-1.01 (m, 3H); UPLC-MS-2a: Rt = 0.78 min; MS m/z [M + H]⁺ 602.5/604.5; C-SFC-3 (mobile phase: CO₂/[IPA + 0.1% Et₃N] 78/22): Rt = 3.15 min, Example 179a: C-SFC-3 (mobile phase: CO₂/[IPA + 0.1% Et₃N] 78/22): Rt = 2.43 min. |
| 180a/ 180b | 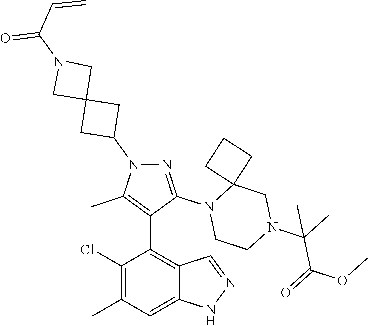 methyl 2-(5-(1-(2-acryloyl-2-azaspiro[3.3]heptan-6-yl)-4- (5-chloro-6-methyl-1H-indazol-4-yl)-5-methyl-1H- pyrazol-3-yl)-5,8-diazaspiro[3.5]nonan-8-yl)- 2-methylpropanoate | Using Method-8 from tert-butyl 6-(4-(5-chloro- 6-methyl-1-(tetrahydro- 2H-pyran-2-yl)-1H- indazol-4-yl)-3-(8-(1- methoxy-2-methyl-1- oxopropan-2-yl)-5,8- diazaspiro[3.5]nonan-5- yl)-5-methyl-1H-pyrazol- 1-yl)-2- azaspiro[3.3]heptane-2- carboxylate (described in the synthesis of Example 190a, Step1)(Step 2) and C- SFC-15 (mobile phase: CO₂/[IPA + 0.025% NH₃] 50/50); Example 180a = 1^{st} eluting isomer, Example 180b = 2^{nd} eluting isomer. | Example 180b: ¹H NMR (600 MHz, DMSO-d₆) δ 12.9 (s, 1H), 7.55 (m, 1H), 7.43 (s, 1H), 6.31 (m, 1H), 6.09 (m, 1H), 5.67 (m, 1H), 4.74 (m, 1H), 4.34 (s, 1H), 4.28 (s, 1H), 4.05 (s, 1H), 3.99 (s, 1H), 3.54 (s, 3H), 3.79- 2.61 (m, 6H), 2.47 (s, 3H), 2.40-2.29 (m, 2H), 2.24-2.16 (m, 3H), 2.11 (m, 1H), 1.98 (s, 3H), 1.75 (m, 1H), 1.61-1.49 (m, 3H), 1.15 (s, 3H), 1.14 (s, 3H); UPLC-MS- 4: Rt = 0.87 min; MS m/z [M + H]⁺ 620.6/622.7; C- SFC-10 (mobile phase: CO₂/[IPA + 0.025% NH₃] 50/50): Rt = 3.43 min, Example 180a: C-SFC- 10 (mobile phase: CO₂/[IPA + 0.025% NH₃] 50/50): Rt = 2.56 min. |
| 181a/ 181b | 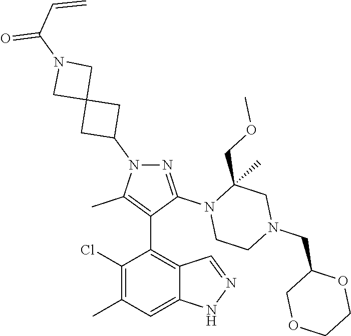 [SMILES: C=CC(=O)N1CC2(CC(n3nc(N4CCN(C[C@@H]5COCCO5)C[C@]4(C)COC)c(-c4c(Cl)c(C)cc5[nH]ncc45)c3C)C2)C1] 1-(6-(3-((R)-4-(((R)-1,4- dioxan-2-yl)methyl)-2- (methoxymethyl)-2- | Using Method-8ab from Intermediate Intermediate C47 (Step 1) and intermediate B1 and C-SFC-4 (mobile phase: CO₂/[IPA + 0.1% Et₃N] 77/23); Example 181a = 1^{st} eluting isomer, Example 181b = 2^{nd} eluting isomer. | Example 181b: ¹H NMR (400 MHz, DMSO-d₆) δ 12.9 (s, 1H), 7.74 (s, 1H), 7.45 (s, 1H), 6.30 (m, 1H), 6.10 (m, 1H), 5.67 (m, 1H), 4.73 (m, 1H), 4.33 (s, 1H), 4.30 (s, 1H), 4.05 (s, 1H), 4.02 (s, 1H), 3.74-3.35 (m, 8H), 3.18- 3.11 (m, 1H), 3.13 (s, 3H), 2.82 (m, 2H), 2.78- 2.61 (m, 4H), 2.47 (s, 3H), 2.25 (m,1H), 2.17- 1.85 (m, 5H), 1.97 (s, 3H), 1.03 (m, 3H). UPLC- MS-4: Rt = 0.68 min; MS m/z [M + H]⁺ 638.6/640.6; C-SFC-3 (mobile phase: CO₂/[IPA + 0.025% NH₃] 76/24): Rt = 3.43 min, Example 181a: C-SFC-3 (mobile phase: CO₂/ [IPA + 0.025% NH₃] 76/24): Rt = 2.42 min. |
| | methylpiperazin-1-yl)-4-(5- | | |
| | chloro-6-methyl-1H-indazol- | | |
| | 4-yl)-5-methyl-1H-pyrazol- | | |
| | 1-yl)-2-azaspiro[3.3]heptan- | | |
| | 2-yl)prop-2-en-1-one | | |
| 182a/ 182b | 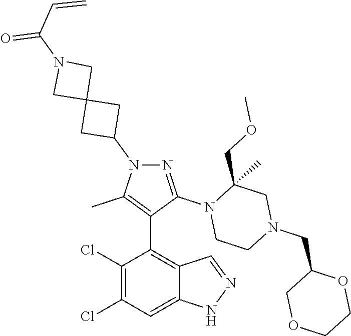 [SMILES: C=CC(=O)N1CC2(CC(n3nc(N4CCN(C[C@@H]5COCCO5)C[C@]4(C)COC)c(-c4c(Cl)c(Cl)cc5[nH]ncc45)c3C)C2)C1] 1-(6-(3-((R)-4-(((R)-1,4-dioxan-2-yl)methyl)-2- (methoxymethyl)-2-methylpiperazin-1-yl)-4- (5,6-dichloro-1H-indazol-4-yl)-5-methyl-1H-pyrazol-1- yl)-2-azaspiro[3.3]heptan-2-yl)prop-2-en-1-one | Using Method-8ab from from Intermediate C68 (Step 1) and intermediate B1 and C- SFC-2 (mobile phase: CO₂/[IPA + 0.1% Et₃N] 75/25); Example 182a = 1^{st} eluting isomer, Example 182b = 2^{nd} eluting isomer. | Example 182b: ¹H NMR (400 MHz, DMSO-d₆) δ 13.2 (s, 1H), 7.87 (s, 1H), 7.79 (s, 1H), 6.30 (m, 1H), 6.10 (m, 1H), 5.67 (m, 1H), 4.75 (m, 1H), 4.33 (s, 1H), 4.30 (s, 1H), 4.05 (s, 1H), 4.02 (s, 1H), 3.73-3.45 (m, 6H), 3.43- 3.34 (m, 2H), 3.19-3.09 (m, 1H), 3.12 (s, 3H), 2.90-2.81 (m, 2H), 2.78- 2.63 (m, 4H), 2.27 (m, 1H), 2.17-1.91 (m, 5H), 1.99 (s, 3H), 1.01 (m, 3H). UPLC-MS-4: Rt = 0.78 min; MS m/z [M + H]⁺ 658.3/660.3/662.3; C- SFC-3 (mobile phase: CO₂/[IPA + 0.025% NH₃] 75/25): Rt = 2.04 min, Example 182a: C-SFC-3 (mobile phase: CO₂/ [IPA + 0.025% NH₃] 75/25): Rt = 1.80 min. |
| 183a/ 183b | 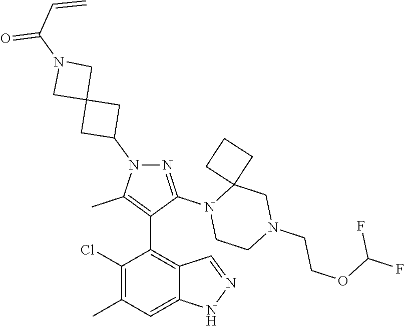 [SMILES: C=CC(=O)N1CC2(CC(n3nc(N4CCN(CCOC(F)F)CC45CCC5)c(-c4c(Cl)c(C)cc5[nH]ncc45)c3C)C2)C1] | Using Method-8 from 1- bromo-2- (difluoromethoxy)ethane (Step 1) and C-SFC-4 (mobile phase: CO₂/ [IPA + 0.025% NH₃] 76/24); Example 183a = 1^{st} eluting isomer, Example 183b = 2^{nd} eluting isomer. | Example 183b: UPLC- MS-2a: Rt = 0.83 min; MS m/z [M + H]⁺ 614.2/616.2; C-SFC-3 (mobile phase: CO₂/[IPA + 0.1% Et₃N] 76/24): Rt = 2.69 min, Example 183a: C-SFC-3 (mobile phase: CO₂/[IPA + 0.1% Et₃N] 76/24): Rt = 1.87 min. |
| | 1-(6-(4-(5-chloro-6-methyl- | | |
| | 1H-indazol-4-yl)-3-(8-(2- | | |
| | (difluoromethoxy)ethyl)-5,8- | | |
| | diazaspiro[3.5]nonan-5-yl)- | | |
| | 5-methyl-1H-pyrazol-1-yl)- | | |
| | 2-azaspiro[3.3]heptan-2- | | |
| | yl)prop-2-en-1-one | | |
| 184a/ 184b | 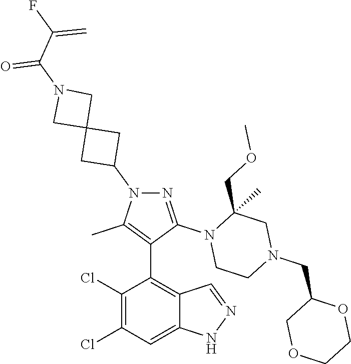 [SMILES: C=C(F)C(=O)N1CC2(CC(n3nc(N4CCN(C[C@@H]5COCCO5)C[C@]4(C)COC)c(-c4c(Cl)c(Cl)cc5[nH]ncc45)c3C)C2)C1] | Using Method-8ab from from Intermediate C68, Intermediate B1 (Step 1) and 2-fluoropropenoic acid (Step 3) and C- SFC-4 (mobile phase: CO₂/[IPA + 0.1% Et₃N] 77/23); Example 184a = 1^{st} eluting isomer, Example 184b = 2^{nd} eluting isomer. | Example 184b: ¹H NMR (400 MHz, DMSO-d₆) δ 13.2 (s, 1H), 7.87 (s, 1H), 7.79 (s, 1H), 5.46 (m, 1H), 5.28 (m, 1H), 4.74 (m, 1H), 4.48 (m, 2H), 4.08 (m, 2H), 3.72-3.34 (m, 8H), 3.16 (m, 1H), 3.13 (s, 3H), 2.88-2.79 (m, 2H), 2.80-2.61 (m, 4H), 2.34-2.23 (m, 1H), 2.18-1.88 (m, 5H), 1.99 (s, 3H), 1.06-0.95 (m, 3H). UPLC-MS-4: Rt = 0.80 min; MS m/z [M + H]⁺ 676.3/678.3/662.3; C- SFC-3 (mobile phase: CO₂/[IPA + 0.025% NH₃] 75/25): Rt = 1.91 min, Example 184a: C-SFC-3 (mobile phase: CO₂/ [IPA + 0.025% NH₃] 75/25): Rt = 1.39 min. |
| | 1-(6-(3-((R)-4-(((R)-1,4- | | |
| | dioxan-2-yl)methyl)-2- | | |
| | (methoxymethyl)-2- | | |
| | methylpiperazin-1-yl)-4- | | |
| | (5,6-dichloro-1H-indazol-4- | | |
| | yl)-5-methyl-1H-pyrazol-1- | | |
| | yl)-2-azaspiro[3.3]heptan-2- | | |
| | yl)-2-fluoroprop-2-en-1-one | | |
| 185a/ 185b | 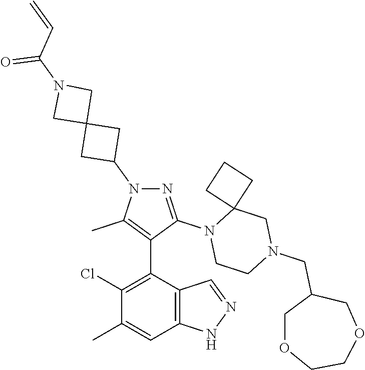 [SMILES: C=CC(=O)N1CC2(CC(n3nc(N4CCN(CC5COCCOC5)CC45CCC5)c(-c4c(Cl)c(C)cc5[nH]ncc45)c3C)C2)C1] | Using Method-8b from Intermediate B3 (Step 1) and C-SFC-4 (mobile phase: CO₂/[IPA + 0.1% Et₃N] 66/34); Example 185a = 1^{st} eluting isomer, Example 185b = 2^{nd} eluting isomer | Example 185b: UPLC- MS-4: Rt = 0.77 min; MS m/z [M + H]⁺ 634.5/636.5; C-SFC-3 (mobile phase: CO₂/[IPA + 0.1% NH₃] 65/35): Rt = 2.08 min, Example 185a: C-SFC-3 (mobile phase: CO₂/[IPA + 0.1% NH₃] 65/35): Rt = 1.09 min. |
| | 1-(6-(3-(8-((1,4-dioxepan-6- | | |
| | yl)methyl)-5,8- | | |
| | diazaspiro[3.5]nonan-5-yl)- | | |
| | 4-(5-chloro-6-methyl-1H- | | |
| | indazol-4-yl)-5-methyl-1H- | | |
| | pyrazol-1-yl)-2- | | |
| | azaspiro [3.3]heptan-2- | | |
| | yl)prop-2-en-1-one | | |
| 186a/ 186b | 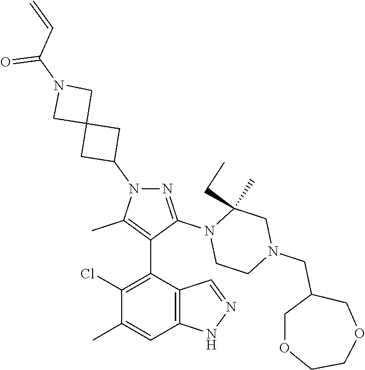 [SMILES: C=CC(=O)N1CC2(CC(n3nc(N4CCN(CC5COCCOC5)C[C@]4(C)CC)c(-c4c(Cl)c(C)cc5[nH]ncc45)c3C)C2)C1] (S)-1-(6-(3-(4-((1,4-dioxepan-6-yl)methyl)-2- ethyl-2-methylpiperazin-1-yl)-4-(5-chloro-6-methyl-1H- indazol-4-yl)-5-methyl-1H-pyrazol-1-yl)-2- azaspiro[3.3]heptan-2-yl)prop-2-en-1-one | Using Method-8b from Intermediate C39 and intermediate B3 (Step 1) and C-SFC-34 (mobile phase: CO₂/[IPA + 0.1% Et₃N] 65/35); Example 186a = 1^{st} eluting isomer, Example 186b = 2^{nd} eluting isomer | Example 186b: ¹H NMR (400 MHz, DMSO-d₆) δ 12.95 (s, 1H), 7.57 (s, 1H), 7.44 (s, 1H), 6.30 (m, 1H), 6.09 (m, 1H), 5.67 (m, 1H), 4.72 (m, 1H), 4.33 (s, 1H), 4.28 (s, 1H), 4.04 (s, 1H), 4.00 (s, 1H), 3.71 (m, 2H), 3.50- 3.66 (m, 4H), 3.41 (m, 2H), 2.98 (m, 1H), 2.80 (m, 1H), 2.59-2.77 (m, 4H), 2.47 (s, 3H), 1.88- 2.19 (m, 6H), 1.95 (s, 3H), 1.79 (m, 2H), 1.66 (m, 1H), 0.91 (m, 3H), 0.64 (m, 3H). UPLC-MS- 4: Rt = 0.74 min; MS m/z [M + H]⁺ 636.5/638.5; C- SFC-3 (mobile phase: CO₂/[IPA + 0.1% NH₃] 75/25): Rt = 4.12 min, Example 186a: C-SFC-3 (mobile phase: CO₂/[IPA + 0.1% NH₃] 75/25): Rt = 2.43 min. |
| 187a/ 187b | 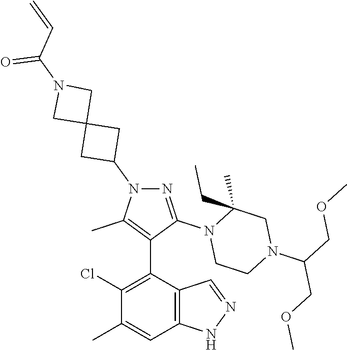 [SMILES: C=CC(=O)N1CC2(CC(n3nc(N4CCN(C(COC)COC)C[C@]4(C)CC)c(-c4c(Cl)c(C)cc5[nH]ncc45)c3C)C2)C1] | Using Method-8b from Intermediate C39 and 2- bromo-1,3- dimethoxypropane (CAS [90321-39-4]) (Step 1) and C-HPLC-10 (mobile phase: [Heptane + 0.1% Et₃N]/ [IPA + 0.1% Et₃N] 88/12); Example 187a = 1^{st} eluting isomer, Example 187b = 2^{nd} eluting isomer | Example 187b: UPLC- MS-4: Rt = 0.78 min; MS m/z [M + H]⁺ 624.5/626.5; C-HPLC-9 (mobile phase: [Heptane + 0.1% DEA]/[IPA + 0.1% DEA] 88/12): Rt = 8.82 min, Example 187a: C-HPLC- 9 (mobile phase: [Heptane + 0.1% DEA]/ [IPA + 0.1% DEA] 88/12): Rt = 6.61 min. |
| | (S)-1-(6-(4-(5-chloro-6- | | |
| | methyl-1H-indazol-4-yl)-3- | | |
| | (4-(1,3-dimethoxypropan-2- | | |
| | yl)-2-ethyl-2- | | |
| | methylpiperazin-1-yl)-5- | | |
| | methyl-1H-pyrazol-1-yl)-2- | | |
| | azaspiro[3.3]heptan-2- | | |
| | yl)prop-2-en-1-one | | |
| 188a/ 188b | 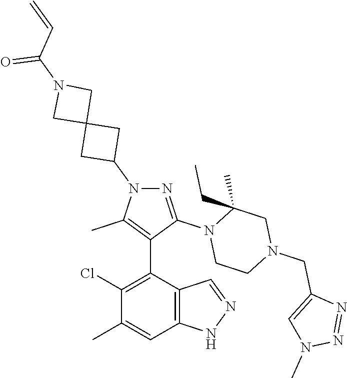 [SMILES: C=CC(=O)N1CC2(CC(n3nc(N4CCN(Cc5cn(C)nn5)C[C@]4(C)CC)c(-c4c(Cl)c(C)cc5[nH]ncc45)c3C)C2)C1] | Using Method-8b from Intermediate C39 and 4- (chloromethyl)-1- methyl-1H-1,2,3-triazole hydrochloride (CAS [327985-63-7]) (Step 1) and C-SFC-30: (mobile phase: CO₂/[IPA + 0.1% Et₃N] 70/30); Example 188a = 1^{st} eluting atropisomer Example 188b = 2^{nd} eluting atropisomer | Example 188b: UPLC- MS-4: Rt = 0.71 min; MS m/z [M + H]⁺ 617.5/619.4; C-SFC-3 (mobile phase: CO₂/[IPA + 0.1% NH₃] 72/28): Rt = 4.04 min, Example 188a: C-SFC-3 (mobile phase: CO₂/[IPA + 0.1% NH₃] 72/28): Rt = 3.21 min. |
| | (S)-1-(6-(4-(5-chloro-6- | | |
| | methyl-1H-indazol-4-yl)-3- | | |
| | (2-ethyl-2-methyl-4-((1- | | |
| | methyl-1H-1,2,3-triazol-4- | | |
| | yl)methyl)piperazin-1-yl)-5- | | |
| | methyl-1H-pyrazol-1-yl)-2- | | |
| | azaspiro[3.3]heptan-2- | | |
| | yl)prop-2-en-1-one | | |
| 189a/ 189b | 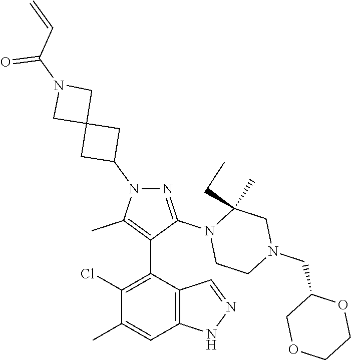 [SMILES: C=CC(=O)N1CC2(CC(n3nc(N4CCN(C[C@H]5COCCO5)C[C@]4(C)CC)c(-c4c(Cl)c(C)cc5[nH]ncc45)c3C)C2)C1] | Using Method-8 from Intermediate C39 and Intermediate B2 (Step 1) and C-SFC-1 (mobile phase: CO₂/IPA 70/30); Example 189a = 1^{st} eluting atropisomer Example 189b = 2^{nd} eluting atropisomer | Example 189b: UPLC- MS-4: Rt = 0.75 min; MS m/z [M + H]⁺ 620.3/622.3; C-SFC-3 (mobile phase: CO₂/IPA 70/30): Rt = 3.38 min, Example 189a: C-SFC-3 (mobile phase: CO₂/IPA 70/30): Rt = 1.96 min. |
| | (S)-1-(6-(3-(8-((1,4-dioxan- | | |
| | 2-yl)methyl)-5,8- | | |
| | diazaspiro[3.5]nonan-5-yl)- | | |
| | 4-(5-chloro-6-methyl-1H- | | |
| | indazol-4-yl)-5-methyl-1H- | | |
| | pyrazol-1-yl)-2- | | |
| | azaspiro[3.3]heptan-2- | | |
| | yl)prop-2-en-1-one | | |

### Examples 190a and 190b: (1-(6-(4-(5-Chloro-6-methyl-1H-indazol-4-yl)-3-(8-(1-hydroxy-2-methylpropan-2-yl)-5,8-diazaspiro[3.5]nonan-5-yl)-5-methyl-1H-pyrazol-1-yl)-2-azaspiro[3.3]heptan-2-yl)prop-2-en-1-one

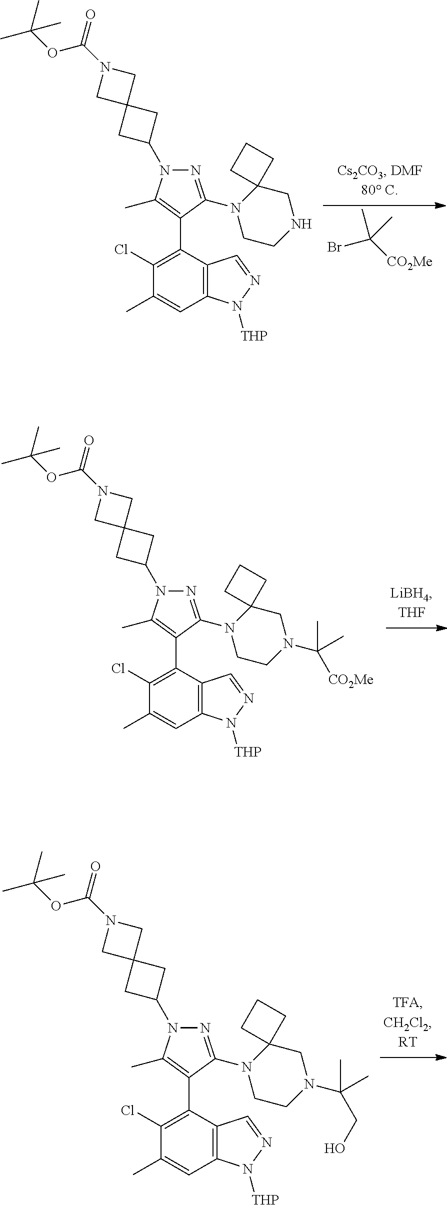

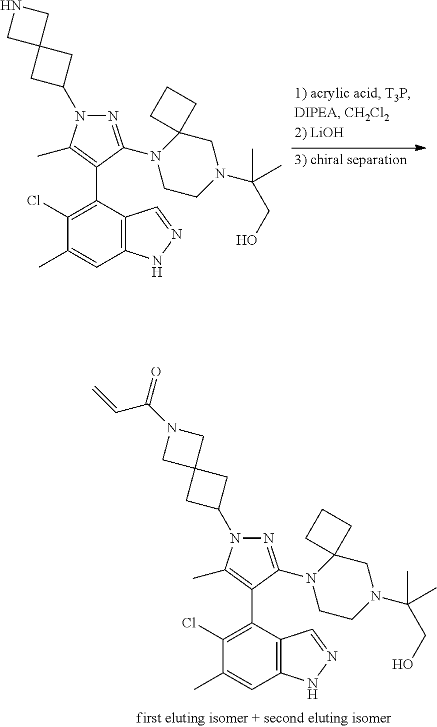

### Step 1: Tert-butyl 6-(4-(5-chloro-6-methyl-1-(tetrahydro-2H-pyran-2-yl)-1H-indazol-4-yl)-3-(8-(1-methoxy-2-methyl-1-oxopropan-2-yl)-5,8-diazaspiro[3.5]nonan-5-yl)-5-methyl-1H-pyrazol-1-yl)-2-azaspiro[3.3]heptane-2-carboxylate

To a suspension of tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate (intermediate C28, 1.52 mmol, 1.00 g) and Cs₂CO₃ (1.49 g, 4.57 mmol) in DMF (10 mL) was added under nitrogen atmosphere methyl alpha-dromoisobutyrate (0.30 mL, 2.30 mmol) and the reaction mixture was stirred at 80° C. for 2 h. Methyl 2-bromo-2-methylpropanoate (0.15 mL, 1.15 mmol) was added again and the RM was further stirred for 1 h at 80° C. (this operation was repeated 3 times) until completion of the reaction. The RM was poured into a sat. aq. NaHCO₃ solution and extracted with EtOAc (×2). The combined organic extracts were washed with brine, dried (Na₂SO₄), filtered and concentrated. The crude residue was purified by normal phase chromatography (eluent: EtOAc in n-heptane 0 to 50%) to give the title compound. UPLC-MS-4: Rt=1.44 min; MS m/z [M+H]⁺ 750.5/752.5.

### Step 2: Tert-butyl 6-(4-(5-chloro-6-methyl-1-(tetrahydro-2H-pyran-2-yl)-1H-indazol-4-yl)-3-(8-(1-hydroxy-2-methylpropan-2-yl)-5,8-diazaspiro[3.5]nonan-5-yl)-5-methyl-1H-pyrazol-1-yl)-2-azaspiro[3.3]heptane-2-carboxylate

To a solution of tert-butyl 6-(4-(5-chloro-6-methyl-1-(tetrahydro-2H-pyran-2-yl)-1H-indazol-4-yl)-3-(8-(1-methoxy-2-methyl-1-oxopropan-2-yl)-5,8-diazaspiro[3.5]nonan-5-yl)-5-methyl-1H-pyrazol-1-yl)-2-azaspiro[3.3]heptane-2-carboxylate (Step 1, 280 mg, 0.37 mmol) in THF (0.50 mL) was added LiBH₄ (2 M in THF, 0.56 mL, 1.12 mmol) and the reaction mixture was stirred at 60° C. for 15 h. LiBH₄ (2 M in THF, 0.56 mL, 1.12 mmol) was added again and the RM further stirred 7 h at 60° C. The RM was carefully poured into an aq. sat. NaHCO₃ solution and extracted with CH₂Cl₂ (×3). The combined organic extracts were dried (phase separator) and concentrated in vacuo. The crude residue was purified by normal phase chromatography (eluent: MeOH in CH₂Cl₂ 0 to 8%) to give the title compound as a white foam. UPLC-MS-4: Rt=1.13 and 1.15 min; MS m/z [M+H]⁺ 722.5/724.5.

### Step 3: 2-(5-(4-(5-Chloro-6-methyl-1H-indazol-4-yl)-5-methyl-1-(2-azaspiro[3.3]heptan-6-yl)-1H-pyrazol-3-yl)-5,8-diazaspiro[3.5]nonan-8-yl)-2-methylpropan-1-ol Isomer 1 and Isomer 2

To a solution of tert-butyl 6-(4-(5-chloro-6-methyl-1-(tetrahydro-2H-pyran-2-yl)-1H-indazol-4-yl)-3-(8-(1-hydroxy-2-methylpropan-2-yl)-5,8-diazaspiro[3.5]nonan-5-yl)-5-methyl-1H-pyrazol-1-yl)-2-azaspiro[3.3]heptane-2-carboxylate (Step 2, 220 mg, 0.31 mmol) in CH₂Cl₂ (1 mL) was added TFA (0.71 mL, 9.14 mmol) and the reaction mixture was stirred at RT for 1.5 h. The reaction mixture was evaporated to dryness to give the title compound as a trifluoroacetate salt which was used without purification in the next step. UPLC-MS-4: Rt=0.47 min; MS m/z [M+H]⁺ 538.3/540.2.

### Step 4: 1-(6-(4-(5-Chloro-6-methyl-1H-indazol-4-yl)-3-(8-(1-hydroxy-2-methylpropan-2-yl)-5,8-diazaspiro[3.5]nonan-5-yl)-5-methyl-1H-pyrazol-1-yl)-2-azaspiro[3.3]heptan-2-yl)prop-2-en-1-one

A solution of acrylic acid (0.045 mL, 0.65 mmol), propylphosphonic anhydride (50% in EtOAc, 385 μL, 0.65 mmol) and DIPEA (0.76 mL, 4.46 mmol) in CH₂Cl₂ (3.90 mL) was stirred at RT for 20 min. This solution was then added to an ice-cooled solution of 2-(5-(4-(5-chloro-6-methyl-1H-indazol-4-yl)-5-methyl-1-(2-azaspiro[3.3]heptan-6-yl)-1H-pyrazol-3-yl)-5,8-diazaspiro[3.5]nonan-8-yl)-2-methylpropan-1-ol (Step 3, 0.30 mmol) in CH₂Cl₂ (1.90 mL) and the reaction mixture was stirred at RT for 15 min. The RM was poured into a sat. aq. NaHCO₃ solution and extracted with CH₂Cl₂ (×3). The combined organic extracts were dried (phase separator) and concentrated in vacuo. This material was dissolved in THF (2.90 mL), LiOH (2 N aqueous, 1.49 mL, 2.97 mmol) was added and the mixture was stirred at RT for 1 h. The RM was poured into a sat. aq. NaHCO₃ solution and extracted with CH₂Cl₂ (×3), the combined organic layers were dried (phase separator) and concentrated in vacuo. The crude residue was purified by normal phase chromatography (eluent: MeOH in CH₂Cl₂ 0 to 15%) to give the title compound. The isomers were separated by chiral SFC (C-SFC-4; mobile phase: CO₂/[IPA+0.1% Et₃N]: 70/30) to give the title compound Example 190b as the second eluting peak: ¹H NMR (600 MHz, DMSO-d₆) δ 12.9 (s, 1H), 7.56 (s, 1H), 7.43 (s, 1H), 6.30 (m, 1H), 6.10 (m, 1H), 5.67 (m, 1H), 4.75 (m, 1H), 4.34 (s, 1H), 4.28 (s, 1H), 4.09-4.05 (m, 2H), 4.00 (m, 1H), 3.19 (s, 2H), 2.81-2.63 (m, 6H), 2.47 (s, 3H), 2.47-2.34 (m, 2H), 2.30-2.11 (m, 4H), 1.99 (s, 3H), 1.73 (m, 1H), 1.62-1.49 (m, 3H), 0.85 (s, 6H); UPLC-MS-2e: Rt=3.35 min; MS m/z [M+H]⁺ 592.3/594.3; C-SFC-3 (mobile phase: CO₂/[IPA+0.025% NH₃]: 70/30): Rt=2.72 min. Example 190a was obtained as the first eluting peak: C-SFC-3 (mobile phase: CO₂/[IPA+0.025% NH₃]: 70/30): Rt=2.07 min.

### Examples 191a and 191b: (1-(6-(4-(5-Chloro-6-methyl-1H-indazol-4-yl)-5-methyl-3-(8-(3-methyloxetan-3-yl)-5,8-diazaspiro[3.5]nonan-5-yl)-1H-pyrazol-1-yl)-2-azaspiro[3.3]heptan-2-yl)prop-2-en-1-one

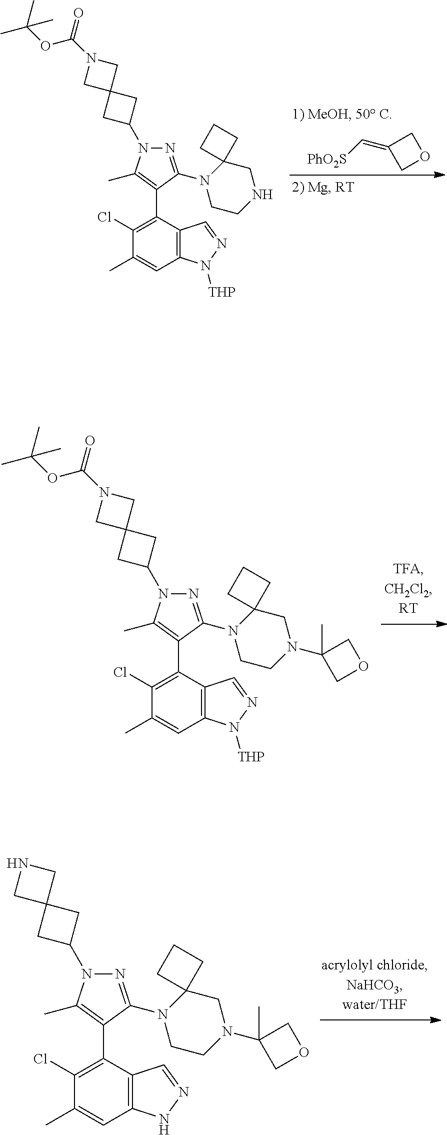

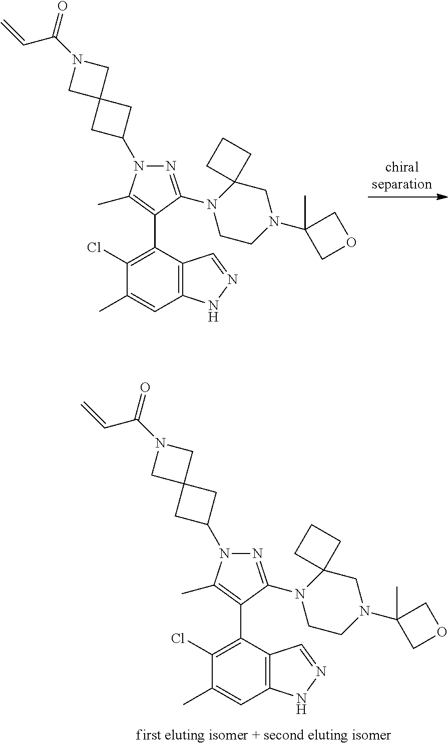 [SMILES: C=CC(=O)N1CC2(CC(n3nc(N4CCN(C5(C)COC5)CC45CCC5)c(-c4c(Cl)c(C)cc5[nH]ncc45)c3C)C2)C1 ; C=CC(=O)N1CC2(CC(n3nc(N4CCN(C5(C)COC5)CC45CCC5)c(-c4c(Cl)c(C)cc5[nH]ncc45)c3C)C2)C1]

### Step 1: Tert-butyl 6-(4-(5-chloro-6-methyl-1-(tetrahydro-2H-pyran-2-yl)-1H-indazol-4-yl)-5-methyl-3-(8-(3-methyloxetan-3-yl)-5,8-diazaspiro[3.5]nonan-5-yl)-1H-pyrazol-1-yl)-2-azaspiro[3.3]heptane-2-carboxylate

To a solution of 3-((phenylsulfonyl)methylene)oxetane (Intermediate B4, 353 mg, 1.68 mmol) in MeOH (10 mL) under argon was added tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate (intermediate C28, 1.34 mmol, 900 mg) and the the reaction mixture was stirred at 50° C. for 72 h. Magnesium (196 mg, 8.06 mmol) was added and the RM was stirred for 20 h. Magnesium (50 mg, 2.06 mmol) was added again and the RM wa further stirred at RT for 16 h to complete the reaction. The RM was diluted with a sat. aq. NaHCO₃ solution, extracted with EtOAc and the combined organic extracts were washed with brine, dried (Na₂SO₄), filtered and evaporated. The crude residue was purified by normal phase chromatography (eluent: MeOH in CH₂Cl₂ 0 to 12%). UPLC-MS-4: Rt=1.35 min; MS m/z [M+H]⁺ 720.3/722.3.

### Step 2: 5-(4-(5-Chloro-6-methyl-1H-indazol-4-yl)-5-methyl-1-(2-azaspiro[3.3]heptan-6-yl)-1H-pyrazol-3-yl)-8-(3-methyloxetan-3-yl)-5,8-diazaspiro[3.5]nonane

To a solution of tert-butyl 6-(4-(5-chloro-6-methyl-1-(tetrahydro-2H-pyran-2-yl)-1H-indazol-4-yl)-5-methyl-3-(8-(3-methyloxetan-3-yl)-5,8-diazaspiro[3.5]nonan-5-yl)-1H-pyrazol-1-yl)-2-azaspiro[3.3]heptane-2-carboxylate (Step 1, 400 mg, 0.47 mmol) in CH₂Cl₂ (10 mL) was added TFA (1.00 mL, 13.0 mmol) and the reaction mixture was stirred at RT for 72 h. The RM was evaporated to dryness to give the title compound as a trifluoroacetate salt which was used without purification in the next step. UPLC-MS-4: Rt=0.49 min min; MS m/z [M+H]⁺ 536.4/538.4.

### Step 3: (1-(6-(4-(5-Chloro-6-methyl-1H-indazol-4-yl)-5-methyl-3-(8-(3-methyloxetan-3-yl)-5,8-diazaspiro[3.5]nonan-5-yl)-1H-pyrazol-1-yl)-2-azaspiro[3.3]heptan-2-yl)prop-2-en-1-one

To a solution of 5-(4-(5-chloro-6-methyl-1H-indazol-4-yl)-5-methyl-1-(2-azaspiro[3.3]heptan-6-yl)-1H-pyrazol-3-yl)-8-(3-methyloxetan-3-yl)-5,8-diazaspiro[3.5]nonane (Step 2, 0.44 mmol) in THF (10 mL) were added water (0.26 mL), NaHCO₃ (0.73 g, 8.73 mmol) followed by acryloyl chloride (0.045 mL, 0.54 mmol). The reaction mixture was stirred at RT for 1 h. After completion of the reaction, LiOH (2M, 2.18 mL, 4.36 mmol) was added and the mixture was stirred at RT for 30 min. The RM was diluted with water, extracted with EtOAc (×2) and the combined organic extracts were washed with brine, dried (Na₂SO₄), filtered and evaporated. The crude residue was purified by reverse phase HPLC (RP-HPLC-1) to give the title compound. The isomers were separated by chiral SFC (C-SFC-4; mobile phase: CO₂/[IPA+0.1% Et₃N]: 72/28) to give the title compound Example 191b as the second eluting peak: ¹H NMR (400 MHz, DMSO-d₆) δ 12.9 (s, 1H), 7.57 (s, 1H), 7.44 (s, 1H), 6.30 (m, 1H), 6.10 (m, 1H), 5.67 (m, 1H), 4.74 (m, 1H), 4.35-4.27 (m, 4H), 4.05 (m, 3H), 3.99 (m, 1H), 2.80-2.65 (m, 6H), 2.47 (s, 3H), 2.27-2.08 (m, 4H), 2.04-1.92 (m, 2H), 1.98 (s, 3H), 1.82 (m, 1H), 1.62-1.54 (m, 3H), 1.14 (s, 3H); UPLC-MS-4: Rt=0.76 min; MS m/z [M+H]⁺ 590.3/592.3; C-SFC-3 (mobile phase: CO₂/[IPA+0.1% Et₃N]: 70/30): Rt=1.97 min. The other isomer Example 191a was obtained as the first eluting peak: C-SFC-3 (mobile phase: CO₂/[IPA+0.1% Et₃N]: 70/30): Rt=1.47 min.

### Examples 192a and 192b: (S)-1-(6-(4-(5-chloro-6-methyl-1H-indazol-4-yl)-3-(2-ethyl-2-methyl-4-(pyridazin-4-yl)piperazin-1-yl)-5-methyl-1H-pyrazol-1-yl)-2-azaspiro[3.3]heptan-2-yl)prop-2-en-1-one

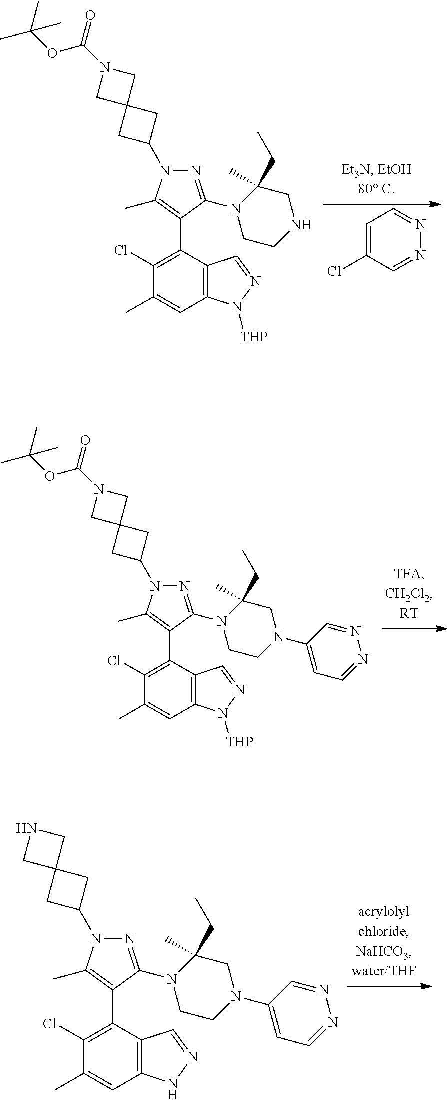

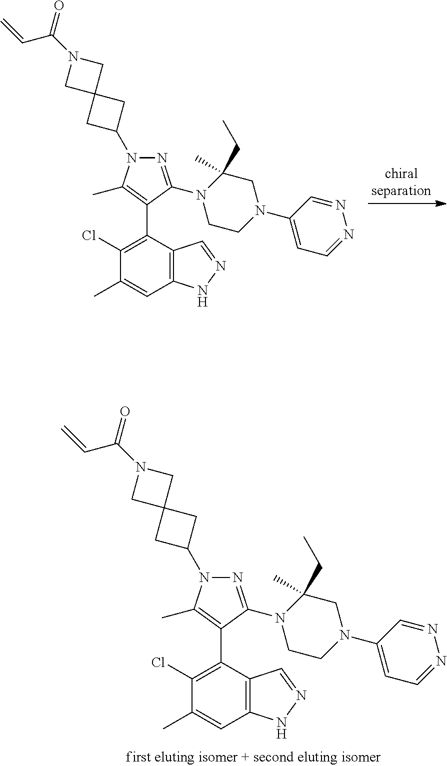 [SMILES: C=CC(=O)N1CC2(CC(n3nc(N4CCN(c5ccnnc5)C[C@]4(C)CC)c(-c4c(Cl)c(C)cc5[nH]ncc45)c3C)C2)C1 ; C=CC(=O)N1CC2(CC(n3nc(N4CCN(c5ccnnc5)C[C@]4(C)CC)c(-c4c(Cl)c(C)cc5[nH]ncc45)c3C)C2)C1]

### Step 1: Tert-butyl 6-(4-(5-chloro-6-methyl-1-(tetrahydro-2H-pyran-2-yl)-1H-indazol-4-yl)-3-((S)-2-ethyl-2-methyl-4-(pyridazin-4-yl)piperazin-1-yl)-5-methyl-1H-pyrazol-1-yl)-2-azaspiro[3.3]heptane-2-carboxylate

To a solution of tert-butyl 6-(4-(5-chloro-6-methyl-1-(tetrahydro-2H-pyran-2-yl)-1H-indazol-4-yl)-3-((S)-2-ethyl-2-methylpiperazin-1-yl)-5-methyl-1H-pyrazol-1-yl)-2-azaspiro[3.3]heptane-2-carboxylate (Intermediate C39, 0.91 mmol, 600 mg) in MeOH (8 mL) were added under argon 4-chloropyridazine hydrochloride (217 mg, 1.37 mmol) and Et₃N (0.70 mL, 5.02 mmol). The reaction mixture was stirred at 80° C. for 18 h. The RM was diluted with a sat. aq. NaHCO₃ solution, extracted with EtOAc and the combined organic extracts were washed with brine, dried (Na₂SO₄), filtered and evaporated. The crude residue was purified by normal phase chromatography (eluent: MeOH in CH₂Cl₂ 0 to 7%). UPLC-MS-4: Rt=1.14 min; MS m/z [M+H]⁺ 730.4/732.4.

### Step 2: (S)-5-chloro-4-(3-(2-ethyl-2-methyl-4-(pyridazin-4-yl)piperazin-1-yl)-5-methyl-1-(2-azaspiro[3.3]heptan-6-yl)-1H-pyrazol-4-yl)-6-methyl-1H-indazole

To a solution of tert-butyl 6-(4-(5-chloro-6-methyl-1-(tetrahydro-2H-pyran-2-yl)-1H-indazol-4-yl)-3-((S)-2-ethyl-2-methyl-4-(pyridazin-4-yl)piperazin-1-yl)-5-methyl-1H-pyrazol-1-yl)-2-azaspiro[3.3]heptane-2-carboxylate (Step 1, 540 mg, 0.72 mmol) in CH₂Cl₂ (15 mL) was added under argon TFA (1.60 mL, 20.8 mmol) and the reaction mixture was stirred at RT for 4 h. The RM was evaporated to dryness to give the title compound as a trifluoroacetate salt which was used without purification in the next step. UPLC-MS-4: Rt=0.49 and 0.51 min min; MS m/z [M+H]⁺ 546.5/548.5.

### Step 3: (S)-1-(6-(4-(5-chloro-6-methyl-1H-indazol-4-yl)-3-(2-ethyl-2-methyl-4-(pyridazin-4-yl)piperazin-1-yl)-5-methyl-1H-pyrazol-1-yl)-2-azaspiro[3.3]heptan-2-yl)prop-2-en-1-one

To a solution of ((S)-5-chloro-4-(3-(2-ethyl-2-methyl-4-(pyridazin-4-yl)piperazin-1-yl)-5-methyl-1-(2-azaspiro[3.3]heptan-6-yl)-1H-pyrazol-4-yl)-6-methyl-1H-indazole (Step 2, 0.71 mmol) in THF (16 mL) were added water (0.41 mL), NaHCO₃ (1.19 g, 14.2 mmol) followed by acryloyl chloride (0.07 mL, 0.84 mmol). The reaction mixture was stirred at RT for 1 h. The RM was diluted with water, extracted with EtOAc (×2) and the combined organic extracts were washed with brine, dried (Na₂SO₄), filtered and evaporated. The crude residue was purified by normal phase chromatography (eluent: MeOH in CH₂Cl₂ 0 to 10%) to give the title compound. The isomers were separated by chiral SFC (C-SFC-4; mobile phase: CO₂/[IPA+0.025% NH₃]: 50/50) to give the title compound Example 192b as the second eluting peak: ¹H NMR (400 MHz, DMSO-d₆) δ 13.0 (s, 1H), 8.88 (br. s, 1H), 8.52 (m, 1H), 7.63 (s, 1H), 7.48 (s, 1H), 6.84 (m, 1H), 6.27 (m, 1H), 6.08 (m, 1H), 5.65 (m, 1H), 4.74 (m, 1H), 4.34 (s, 1H), 4.27 (s, 1H), 4.04 (s, 1H), 3.99 (m, 1H), 3.28-3.15 (m, 3H), 3.13-3.96 (m, 3H), 3.78-2.62 (m, 4H), 2.49 (s, 3H), 1.99 (s, 3H), 1.71 (m, 1H), 1.55 (m, 1H), 0.93 (br s, 3H), 0.60 (t, 3H); UPLC-MS-4: Rt=0.79 min; MS m/z [M+H]⁺ 600.4/602.5; C-SFC-3 (mobile phase: CO₂/[IPA+0.1% Et₃N]: 70/30): Rt=2.48 min. The other isomer Example 192a was obtained as the first eluting peak: C-SFC-3 (mobile phase: CO₂/[IPA+0.1% Et₃N]: 70/30): Rt=0.81 min.

### Method-9 for the Preparation of Examples 193a and 193b: 1-(6-(4-(5-Chloro-6-methyl-1H-indazol-4-yl)-3-(2,2-dimethyl-4-(1-methyl-1H-pyrazol-4-yl)piperazin-1-yl)-5-methyl-1H-pyrazol-1-yl)-2-azaspiro[3.3]heptan-2-yl)prop-2-en-1-one

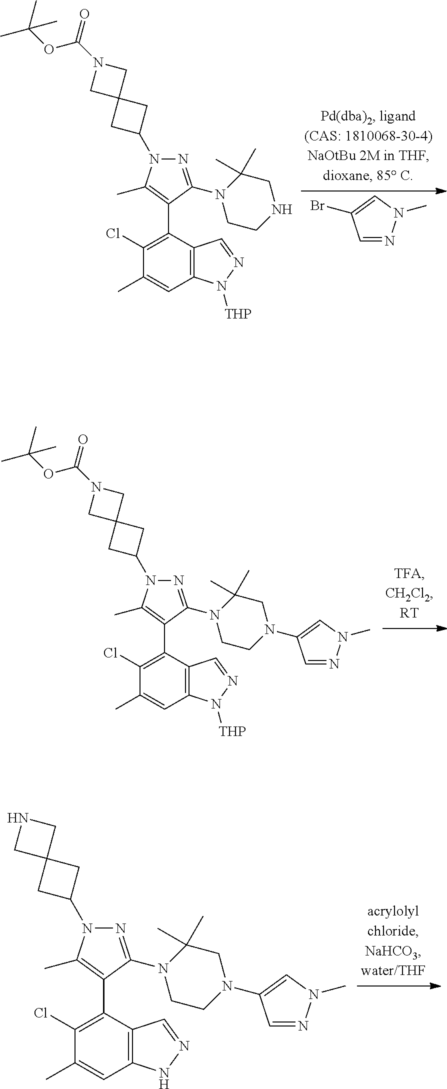

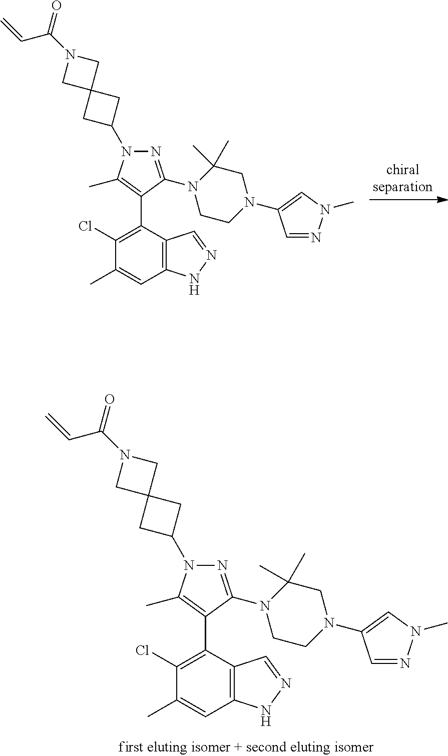

### Step 1: Tert-butyl 6-(4-(5-chloro-6-methyl-1-(tetrahydro-2H-pyran-2-yl)-1H-indazol-4-yl)-3-(2,2-dimethyl-4-(1-methyl-1H-pyrazol-4-yl)piperazin-1-yl)-5-methyl-1H-pyrazol-1-yl)-2-azaspiro[3.3]heptane-2-carboxylate

To a mixture of tert-butyl 6-(4-(5-chloro-6-methyl-1-(tetrahydro-2H-pyran-2-yl)-1H-indazol-4-yl)-3-(2,2-dimethylpiperazin-1-yl)-5-methyl-1H-pyrazol-1-yl)-2-azaspiro[3.3]heptane-2-carboxylate (Intermediate C38, 400 mg, 0.63 mmol), 4-bromo-1-methyl-1H-pyrazole (131 mg, 0.81 mmol), Pd(dba)₂ (36.0 mg, 0.063 mmol) and bis(3,5-bis(trifluoromethyl)phenyl)(2′,6′-bis(dimethylamino)-3,6-dimethoxybiphenyl-2-yl)phosphine (CAS: 1810068-30-4, 52.2 mg, 0.069 mmol) in 1,4-dioxane (3.13 mL) was added NaOtBu (2M in THF, 439 μL, 0.88 mmol) and the reaction mixture was stirred at 85° C. for 16 h. After cooling to RT, the RM was poured into an aq. sat. NaHCO₃ solution and extracted with CH₂Cl₂ (×3). The combined organic layers were dried (phase separator) and concentrated under reduced pressure. The crude residue was diluted with THF (10 mL), SiliaMetS®Thiol (0.26 mmol) was added and the mixture swirled for 1 h at 40° C. The mixture was filtered, the filtrate was concentrated and the crude residue was purified by normal phase chromatography (eluent: MeOH in CH₂Cl₂ 0 to 5%) to give the title compound as a orange foam. UPLC-MS-2a: Rt=1.28 min; MS m/z [M+H]⁺ 718.4/720.4.

### Step 2: 5-Chloro-4-(3-(2,2-dimethyl-4-(1-methyl-1H-pyrazol-4-yl)piperazin-1-yl)-5-methyl-1-(2-azaspiro[3.3]heptan-6-yl)-1H-pyrazol-4-yl)-6-methyl-1H-indazole

To tert-butyl 6-(4-(5-chloro-6-methyl-1-(tetrahydro-2H-pyran-2-yl)-1H-indazol-4-yl)-3-(2,2-dimethyl-4-(1-methyl-1H-pyrazol-4-yl)piperazin-1-yl)-5-methyl-1H-pyrazol-1-yl)-2-azaspiro[3.3]heptane-2-carboxylate (Step 1, 162 mg, 0.23 mmol) in CH₂Cl₂ (1.10 mL) was added TFA (521 μL, 6.77 mmol) and the reaction mixture was stirred at RT for 1 h. The RM was concentrated, dioxane was added and the mixture was frozen and lyophilised to give the title compound as a trifluoroacetate salt which was used in the next step without further purification. UPLC-MS-2a: Rt=0.78 min; MS m/z [M+H]⁺ 534.3/536.3.

### Step 3: 1-(6-(4-(5-Chloro-6-methyl-1H-indazol-4-yl)-3-(2,2-dimethyl-4-(1-methyl-1H-pyrazol-4-yl)piperazin-1-yl)-5-methyl-1H-pyrazol-1-yl)-2-azaspiro[3.3]heptan-2-yl)prop-2-en-1-one

To a solution of 5-chloro-4-(3-(2,2-dimethyl-4-(1-methyl-1H-pyrazol-4-yl)piperazin-1-yl)-5-methyl-1-(2-azaspiro[3.3]heptan-6-yl)-1H-pyrazol-4-yl)-6-methyl-1H-indazole trifluoroacetate in THF (9.0 mL) at 0° C. were added slowly NaHCO₃ (0.5 M aqueous, 4.51 mL, 2.26 mmol) and a solution of acryloyl chloride (19.2 μL, 0.24 mmol) in THF (100 μL). The reaction mixture was stirred at 0° C. for 1 h, then diluted with CH₂Cl₂ and quenched with a sat. aq. NaHCO₃ solution. The combined organic layers were dried and concentrated. The crude residue was purified by normal phase chromatography (eluent: MeOH in CH₂Cl₂ 0 to 10%) to give the title compound as a yellow foam. The isomers were separated by chiral SFC (C-SFC-7: mobile phase: CO₂/IPA: 50/50). The purified fractions were concentrated to give the title compound Example 193b as the second eluting peak: ¹H NMR (400 MHz, DMSO-d₆) δ 12.95 (s, 1H), 7.59 (s, 1H), 7.44 (s, 1H), 7.10 (s, 1H), 7.01 (s, 1H), 6.40-6.22 (m, 1H), 6.15-6.02 (m, 1H), 5.73-5.57 (m, 1H), 4.88-4.62 (m, 1H), 4.34 (s, 1H), 4.30 (s, 1H), 4.06 (s, 1H), 4.01 (s, 1H), 3.65 (s, 3H), 2.98-2.89 (m, 2H), 2.80-2.69 (m, 4H), 2.47 (s, 3H), 2.42-2.38 (m, 2H), 2.35-2.29 (m, 2H), 2.00 (s, 3H), 1.20 (s, 3H), 1.12 (s, 3H); UPLC-MS-4: Rt=0.98 min; MS m/z [M+H]⁺ 588.5 m 590.5; C-SFC-8 (mobile phase: CO₂/PA: 50/50): Rt=2.36 m. The other isomer Example 193a was obtained as the first eluting peak: C-SFC-8 (mobile phase: CO₂/IPA: 50/50): Rt=1.65 m.

Method-9a: similar to Method-9 except that Step 3 was performed using acrylic acid and T₃P in CH₂Cl₂ as described in Step 3 in Method-7.

The examples 194a and 194b were prepared using analogous methods to Method-9 from intermediates (in Step 1) described in the intermediates synthesis section.

| | | Method, | |
|---|---|---|---|
| | | intermediates | |
| | | (Step 1) and | |
| | | achiral or | |
| | | chiral | |
| | | separation | |
| | | conditions | |
| | | used and order | |
| Example | Structure | of elution | Characterizing data |
| 194a/ 194b | 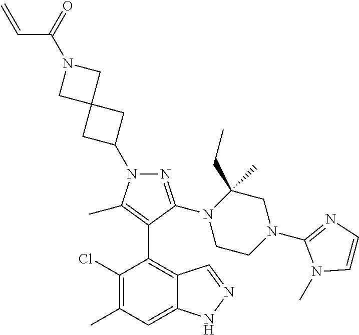 [SMILES: C=CC(=O)N1CC2(CC(n3nc(N4CCN(c5nccn5C)C[C@]4(C)CC)c(-c4c(Cl)c(C)cc5[nH]ncc45)c3C)C2)C1] (S)-1-(6-(4-(5-chloro-6- methyl-1H-indazol-4-yl)- 3-(2-ethyl-2-methyl-4-(1- methyl-1H-imidazol-2- yl)piperazin-1-yl)-5- methyl-1H-pyrazol-1-yl)- 2-azaspiro[3.3]heptan-2- yl)prop-2-en-1-one | Using Method- 9a from Intermediate from Intermediate C39 and 2- bromo-1- methyl-1H- imidazole (CAS [16681-59-7]) (Step 1) and C- SFC-5 (mobile phase: CO₂/[IPA + 0.1% Et₃N] 65/35): Example 194a = 1st eluting isomer, Example 194b = 2nd eluting isomer | Example 194a: ¹H NMR (400 MHz, DMSO-d₆) δ 13.00 (s, 1H), 7.62 (s, 1H), 7.46 (s, 1H), 6.80 (s, 1H), 6.52 (m, 1H), 6.31 (m, 1H), 6.10 (m, 1H), 5.67 (m, 1H), 4.75 (m, 1H), 4.34 (s, 1H), 4.31 (s, 1H), 4.05 (s, 1H), 4.01 (s, 1H), 3.40 (s, 3H), 3.14-2.99 (m, 2H), 2.80-2.65 (m, 6H), 2.61-2.55 (m, 2H), 2.47 (s, 3H), 1.98 (s, 3H), 1.80 (m, 2H), 1.04 (d, 3H), 0.62 (m, 3H). UPLC-MS-4: Rt = 0.77 min; MS m/z [M + H]⁺ 602.5/604.5; C-SFC-6 (mobile phase: CO₂/[IPA + 0.025% NH₃] 65/35): Rt = 3.00 min. Example 194b: C-SFC-6 (mobile phase: CO₂/[IPA + 0.025% NH₃] 65/35): Rt = 3.94 min. |

### Method-10 for the Preparation of Examples 195a and 195b: 1-(6-(3-(4-Acetylpiperazin-1-yl)-4-(5-chloro-1H-indazol-4-yl)-5-methyl-1H-pyrazol-1-yl)-1-methyl-2-azaspiro[3.3]heptan-2-yl)prop-2-en-1-one

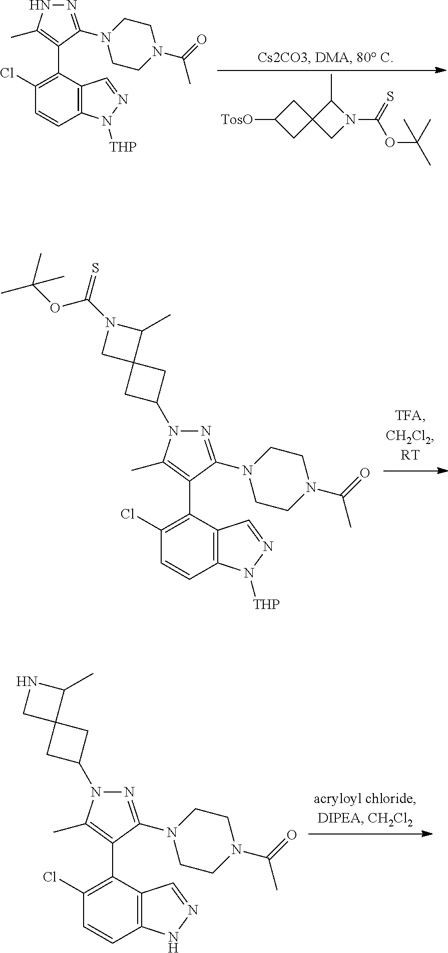

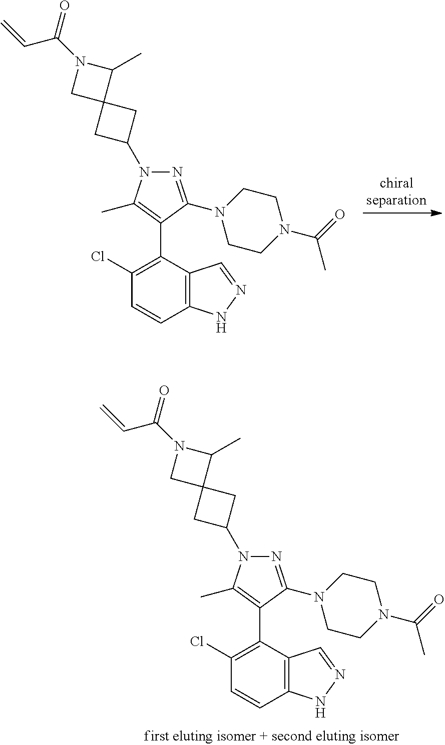 [SMILES: C=CC(=O)N1CC2(CC(n3nc(N4CCN(C(C)=O)CC4)c(-c4c(Cl)ccc5[nH]ncc45)c3C)C2)C1C ; C=CC(=O)N1CC2(CC(n3nc(N4CCN(C(C)=O)CC4)c(-c4c(Cl)ccc5[nH]ncc45)c3C)C2)C1C]

### Step 1: O-(Tert-butyl) 6-(3-(4-acetylpiperazin-1-yl)-4-(5-chloro-1-(tetrahydro-2H-pyran-2-yl)-1H-indazol-4-yl)-5-methyl-1H-pyrazol-1-yl)-1-methyl-2-azaspiro[3.3]heptane-2-carbothioate

To a solution of 1-(4-(4-(5-chloro-1-(tetrahydro-2H-pyran-2-yl)-1H-indazol-4-yl)-5-methyl-1H-pyrazol-3-yl)piperazin-1-yl)ethan-1-one (intermediate C15, 170 mg, 0.38 mmol) and O-(tert-butyl) 1-methyl-6-(tosyloxy)-2-azaspiro[3.3]heptane-2-carbothioate (intermediate C6a, 160 mg, 0.40 mmol) in anhydrous DMA (2.65 mL) was added cesium carbonate (250 mg, 0.77 mmol) and the reaction mixture was heated at 80° C. under nitrogen atmosphere for 16 h. The RM was poured into a sat. aq. NaHCO₃ solution and extracted with EtOAc (×3). The combined organic extracts were washed with water (×2), dried (phase separator), evaporated and the crude residue was purified by normal phase chromatography (eluent: MeOH in CH₂Cl₂ 0 to 10%) to give the title compound as a yellow oil and as the major regioisomer. UPLC-MS-1a: Rt=1.41 and 1.43 min; MS m/z [M+H]⁺ 668.5/670.5.

### Step 2: 1-(4-(4-(5-Chloro-1H-indazol-4-yl)-5-methyl-1-(1-methyl-2-azaspiro[3.3]heptan-6-yl)-1H-pyrazol-3-yl)piperazin-1-yl)ethan-1-one

To a solution of O-(tert-butyl) 6-(3-(4-acetylpiperazin-1-yl)-4-(5-chloro-1-(tetrahydro-2H-pyran-2-yl)-1H-indazol-4-yl)-5-methyl-1H-pyrazol-1-yl)-1-methyl-2-azaspiro[3.3]heptane-2-carbothioate (Step 1, 68 mg, 0.10 mmol) in CH₂Cl₂ (0.48 mL) was added TFA (223 μL, 2.90 mmol) and the reaction mixture was stirred at RT for 2 h. The reaction mixture was poured into water and extracted twice with CH₂Cl₂. The aqueous layer was freezed and lyophilized to give the TFA salt of the title compound which was used directly in the next step. UPLC-MS-1a: Rt=0.61 min; MS m/z [M+H]⁺ 468.4/470.5.

### Step 3: 1-(6-(3-(4-Acetylpiperazin-1-yl)-4-(5-chloro-1H-indazol-4-yl)-5-methyl-1H-pyrazol-1-yl)-1-methyl-2-azaspiro[3.3]heptan-2-yl)prop-2-en-1-one

To an ice-cooled cloudy solution of 1-(4-(4-(5-Chloro-1H-indazol-4-yl)-5-methyl-1-(1-methyl-2-azaspiro[3.3]heptan-6-yl)-1H-pyrazol-3-yl)piperazin-1-yl)ethan-1-one trifluoroacetate (Step 2, 0.07 mmol) in CH₂Cl₂ (890 μL), was added DIPEA (46 μL, 0.26 mmol) followed by a solution of acryloyl chloride (6.10 μL, 0.07 mmol) in CH₂Cl₂ (0.21 mL). The reaction mixture was stirred at 0-5° C. for 20 min. The RM was quenched at 0-5° C. with MeOH (few drops), and stirred at that temperature for 30 min. A sat. aq. NaHCO₃ solution and CH₂Cl₂ were added, the layers were extracted and the aqueous layer was back-extracted with CH₂Cl₂ (×2). The combined organic extracts were dried (phase separator), evaporated and the crude residue was purified by normal phase chromatography (eluent: MeOH in CH₂Cl₂ 0 to 10%) to give the title compound. The isomers were separated by chiral HPLC (C-HPLC-13: mobile phase: n-heptane/CH₂Cl₂/IPA 60:20:20). The purified fractions were concentrated, dioxane was added and the the material was lyophilized to give the title compound Example 195a as the first eluting peak (white powder): ¹H NMR (400 MHz, DMSO-d₆) δ mixture of rotamers: 13.2 (s, 1H), 7.73 (s, 1H), 7.53 (d, 1H), 7.45 (d, 1H), 6.36-6.27 (m, 1H), 6.11 (m, 1H), 5.67 (dd, 1H), 4.73 (m, 1H), 4.46 (m, 0.5H), 4.32-4.24 (m, 1.5H), 3.99 (m, 1H), 3.31-3.12 (m, 4H), 2.89-2.63 (m, 7H), 2.45 (m, 1H), 1.95 (s, 3H), 1.89 (s, 3H), 1.40 (d, 1.5H), 1.35 (d, 1.5H); UPLC-MS-1a: Rt=0.85 min; MS m/z [M+H]⁺ 522.3/524.3; C-HPLC-16 (mobile phase: n-heptane/CH₂Cl₂/IPA 60:20:20): Rt=8.49 min. The other isomer Example 195b was obtained as the second eluting peak: UPLC-MS-1a: Rt=0.86 min; MS m/z [M+H]⁺ 522.3/524.3, C-HPLC-16 (mobile phase: n-heptane/CH₂Cl₂/IPA 60:20:20): Rt=10.94 min.

Method-10a: similar to Method-10 except that Step 1 was performed in DMF at 100° C. instead of DMA.

Method-10b: similar to Method-10 except that Step 3 was performed using Et₃N, acrylic acid and T₃P in CH₂Cl₂ as described in Step 5 in Method-12.

The following examples 196a to 201b were prepared using analogous methods to Method-0 from intermediates (in Step 1) described in the intermediates synthesis section.

In Step 3, If observed the side product resulting from reaction of the acryloyl chloride with the indazole NH was hydrolyzed by treatment with LiOH as described in Method-5 or in Method-12.

When the final separation of isomers is performed by preparative HPLC using acidic conditions the purified fractions were extracted with a sat. aq. NaHCO₃ solution before lyophilization from a mixture of CH₃CN/H₂O to give the title compound as a free base.

| | | Method, | |
|---|---|---|---|
| | | intermediates | |
| | | (Step 1) and | |
| | | achiral or chiral | |
| | | separation | |
| | | conditions used | |
| | | and order of | |
| Example | Structure | elution | Characterizing data |
| 196a/196b | 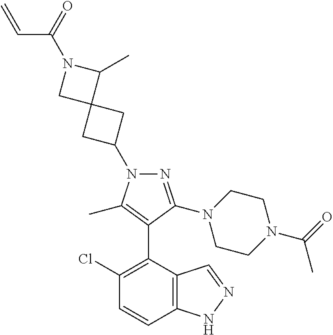 [SMILES: C=CC(=O)N1CC2(CC(n3nc(N4CCN(C(C)=O)CC4)c(-c4c(Cl)ccc5[nH]ncc45)c3C)C2)C1C] 1-(6-(3-(4-acetylpiperazin- 1-yl)-4-(5-chloro-1H- indazol-4-yl)-5-methyl-1H- pyrazol-1-yl)-1-methyl-2- azaspiro[3.3]heptan-2- yl)prop-2-en-1-one | Using Method-10 from Intermediate C6c (Step 1) and C- SFC-5 (mobile phase: CO₂/IPA 60/40): Example 196a = 1st eluting isomer, Example 196b = 2nd eluting isomer | Example 196b: ¹H NMR (600 MHz, DMSO-d₆) δ mixture of rotamers: 13.2 (s, 1H), 7.73 (s, 1H), 7.52 (d, 1H), 7.45 (d, 1H), 6.36 (dd, 0.5H), 6.26 (dd, 0.5H), 6.11 (m, 1H), 5.66 (dd, 1H), 4.72 (m, 1H), 4.46 (m, 0.5H), 4.31- 4.23 (m, 1.5H), 3.99 (m, 1H), 3.28-3.22 (m, 3.5H), 3.15 (m, 0.5H), 2.88-2.79 (m, 2H), 2.76-2.63 (m, 5H), 2.45 (m, 1H), 1.95 (s, 3H), 1.89 (s, 3H), 1.39 (d, 1.5H), 1.35 (d, 1.5H); UPLC-MS-1a: Rt = 0.87 min; MS m/z [M + H]⁺ 522.4/524.4; C-SFC-6 (mobile phase: CO₂/IPA 60/40): Rt = 2.39 min. Example 196a: C-SFC-6 (mobile phase: CO₂/IPA 60/40): Rt = 1.84 min. |
| 197a/197 | 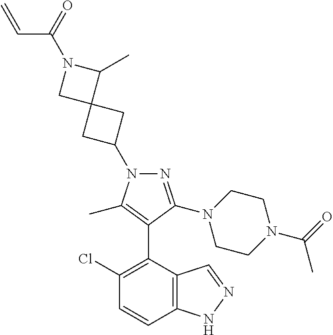 [SMILES: C=CC(=O)N1CC2(CC(n3nc(N4CCN(C(C)=O)CC4)c(-c4c(Cl)ccc5[nH]ncc45)c3C)C2)C1C] 1-(6-(3-(4-acetylpiperazin- 1-yl)-4-(5-chloro-1H- indazol-4-yl)-5-methyl-1H- pyrazol-1-yl)-1-methyl-2- azaspiro[3.3]heptan-2- yl)prop-2-en-1-one | Using Method-10 from Intermediate C6b (Step 1) and C- SFC-2 (mobile phase: CO₂/MeOH 80/20): Example 197a = 1st eluting isomer, Example 197b = 2nd eluting isomer | Example 197a: UPLC-MS- 1a: Rt = 0.86 min; MS m/z [M + H]⁺ 522.4/524.4; C-SFC- 3 (mobile phase: CO₂/MeOH 75/25): Rt = 1.68 min. Example 197b: C-SFC-3 (mobile phase: CO₂/MeOH 75/25): Rt = 2.42 min. |
| 198a/198b | 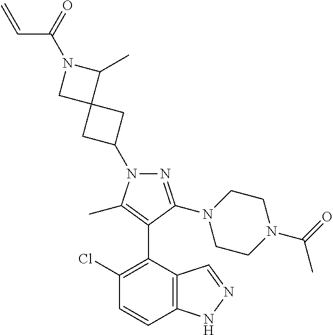 [SMILES: C=CC(=O)N1CC2(CC(n3nc(N4CCN(C(C)=O)CC4)c(-c4c(Cl)ccc5[nH]ncc45)c3C)C2)C1C] 1-(6-(3-(4-acetylpiperazin- 1-yl)-4-(5-chloro-1H- indazol-4-yl)-5-methyl-1H- pyrazol-1-yl)-1-methyl-2- azaspiro[3.3]heptan-2- yl)prop-2-en-1-one | Using Method-10 from Intermediate C6d (Step 1) and C- HPLC-13 (mobile phase: n- heptane/CH₂Cl₂/IPA 60/20/20): Example 198a = 1st eluting isomer, Example 198b = 2nd eluting isomer | Example 198a: UPLC-MS- 1a: Rt = 0.86 min; MS m/z [M + H]⁺ 522.4/524.4; C- HPLC-20 (mobile phase: n- heptane/CH₂Cl₂/IPA 60/20/20): Rt = 8.24 min. Example 198b: C-HPLC-20 (mobile phase: n- heptane/CH₂Cl₂/IPA 60/20/20): Rt = 10.44 min. |
| 199a/199b | 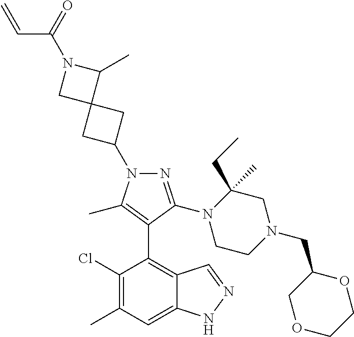 [SMILES: C=CC(=O)N1CC2(CC(n3nc(N4CCN(C[C@@H]5COCCO5)C[C@]4(C)CC)c(-c4c(Cl)c(C)cc5[nH]ncc45)c3C)C2)C1C] 1-(6-(3-((S)-4-(((R)-1,4- dioxan-2-yl)methyl)-2- ethyl-2-methylpiperazin-1- yl)-4-(5-chloro-6-methyl- 1H-indazol-4-yl)-5-methyl- 1H-pyrazol-1-yl)-1-methyl- 2-azaspiro[3.3]heptan-2- yl)prop-2-en-1-one | Using Method-10ab from Intermediate C16 and C8 (Step 1) and C-SFC-4 (mobile phase: CO₂/[IPA + 0.1% Et₃N] 75/25): Example 199a = 1st eluting isomer, Example 199b = 2nd eluting isomer. | Example 199b: UPLC-MS- 2e: Rt = 3.60 min; MS m/z [M+H]+ 636.3/638.3; C-SFC- 3 (mobile phase: CO₂/[IPA + 0.025% NH₃] 75/25): Rt = 2.63 min, Example 199a: C-SFC-3 (mobile phase: CO₂/[IPA + 0.025% NH₃] 75/25): Rt = 1.81 min. |
| 200a/200b | 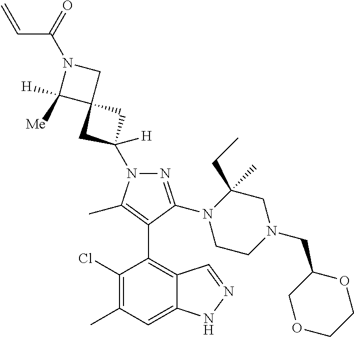 [SMILES: C=CC(=O)N1C[C@]2(C[C@H](n3nc(N4CCN(C[C@@H]5COCCO5)C[C@]4(C)CC)c(-c4c(Cl)c(C)cc5[nH]ncc45)c3C)C2)[C@H]1C] 1-(6-(3-((S)-4-(((R)-1,4- dioxan-2-yl)methyl)-2- ethyl-2-methylpiperazin-1- yl)-4-(5-chloro-6-methyl- 1H-indazol-4-yl)-5-methyl- 1H-pyrazol-1-yl)-1-methyl- 2-azaspiro[3.3]heptan-2- yl)prop-2-en-1-one | Using Method-10ab from Intermediate C16 and C7 (Step 1) and C-SFC-4 (mobile phase: CO₂/[IPA + 0.1% Et₃N] 75/25); Example 200a = 1st eluting isomer, Example 200b = 2nd eluting isomer. | Example 200b: ¹H NMR (400 MHz, DMSO-d₆) δ 12.9 (s, 1H), 7.57 (s, 1H), 7.44 (s, 1H), 6.39-6.23 (m, 1H), 6.11 (m, 1H), 5.66 (m, 1H), 4.74 (m, 1H), 4.45 (m, 0.5H), 4.31- 4.20 (m, 1.5H), 3.99 (s, 1H), 3.72-3.43 (m, 5H), 3.43-3.33 (m, 1H), 3.15 (t, 1H), 2.99- 2.87 (m, 1H), 2.84-2.63 (m, 3H), 2.62-2.50 (m, 2H), 2.48 (s, 3H), 2.17-1.88 (m, 6H), 1.97 (s, 3H), 1.87-1.75 (m, 1H), 1.73-1.62 (m, 1H), 1.40 (d, 1.5H), 1.36 (d, 1.5H), 0.95 (s, 3H), 0.66 (t, 3H). UPLC- MS-2e: Rt = 3.58 min; MS m/z [M + H]⁺ 636.1/638.3; C- SFC-3 (mobile phase: CO₂/[IPA + 0.025% NH₃] 75/25): Rt = 2.85 min, Example 200a: C-SFC-3 (mobile phase: CO₂/[IPA + 0.025% NH₃] 75/25): Rt = 1.88 min. |
| 201a/201b | 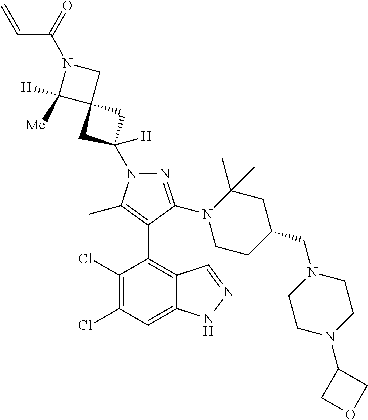 [SMILES: C=CC(=O)N1C[C@]2(C[C@H](n3nc(N4CC[C@@H](CN5CCN(C6COC6)CC5)CC4(C)C)c(-c4c(Cl)c(Cl)cc5[nH]ncc45)c3C)C2)[C@H]1C] 1-(6-(4-(5,6-dichloro-1H- indazol-4-yl)-3-((R)-2,2- dimethyl-4-((4-(oxetan-3- yl)piperazin-1- yl)methyl)piperidin-1-yl)-5- methyl-1H-pyrazol-1-yl)-1- methyl-2- azaspiro[3.3]heptan-2- yl)prop-2-en-1-one | Using Method-10ab from Intermediate C17 and C7 (Step 1) and RP-HPLC-2 (mobile phase: CH₃CN in [H₂O + 0.1% TFA] 10 to 40%); Example 201a = 1st eluting isomer, Example 201b = 2nd eluting isomer. | Example 201b: ¹H NMR (400 MHz, DMSO-d₆) δ 13.2 (s, 1H), 7.78 (s, 1H), 7.64 (s, 1H), 6.39-6.22 (m, 1H), 6.11 (m, 1H), 5.67 (m, 1H), 4.78 (m, 1H), 4.54-4.21 (m, 6H), 3.99 (s, 1H), 3.34 (m, 1H), 3.15 (m, 1H), 2.87-2.61 (m, 5H), 2.39-2.10 (m, 8H), 2.00 (s, 3H), 1.96-1.87 (m, 2H), 1.76 (m, 1H), 1.51 (m, 1H), 1.39 (d, 1.5H), 1.35 (d, 1.5H), 1.42-1.29 (m, 1H), 1.13 (s, 3H), 0.74 (s, 3H), 0.77-0.59 (m, 2H). UPLC-MS-2e: Rt = 4.06 min; MS m/z [M + H]⁺ 695.3/697.3/699.3, Example 201a: UPLC-MS- 2e: Rt = 3.68 min; MS m/z [M + H]⁺ 695.3/697.3/699.3. |

### Method-11 for the Preparation of Examples 202a and 202b: 1-(6-(4-(5-Chloro-6-methyl-1H-indazol-4-yl)-3-(4-(3-methoxyazetidin-1-yl)-2,2-dimethylpiperidin-1-yl)-5-methyl-1H-pyrazol-1-yl)-2-azaspiro[3.3]heptan-2-yl)prop-2-en-1-one

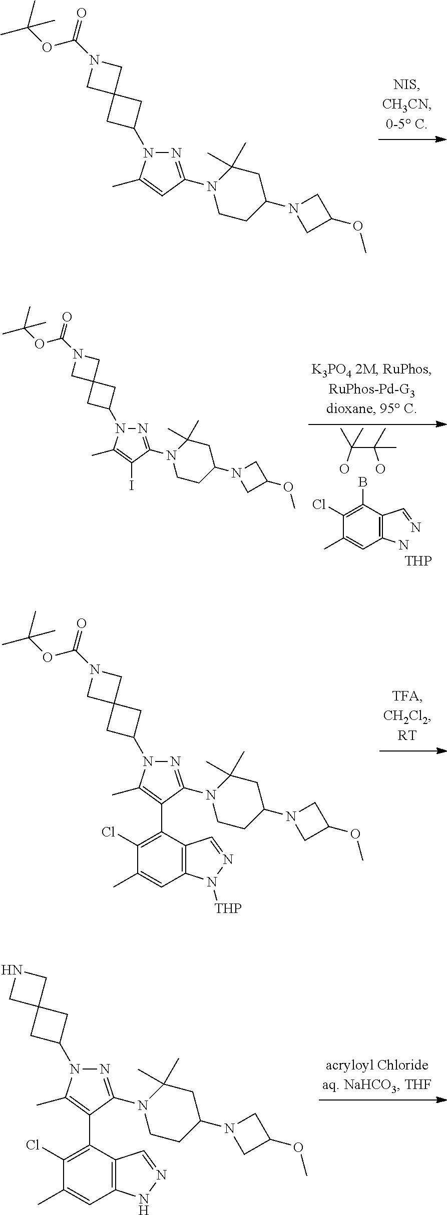

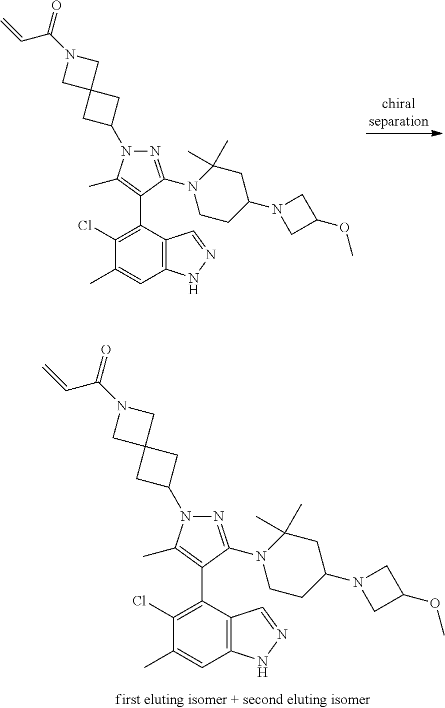

### Step 1: Tert-butyl 6-(4-iodo-3-(4-(3-methoxyazetidin-1-yl)-2,2-dimethylpiperidin-1-yl)-5-methyl-1H-pyrazol-1-yl)-2-azaspiro[3.3]heptane-2-carboxylate

To a solution of tert-butyl 6-(3-(4-(3-methoxyazetidin-1-yl)-2,2-dimethylpiperidin-1-yl)-5-methyl-1H-pyrazol-1-yl)-2-azaspiro[3.3]heptane-2-carboxylate isomer 1 (Intermediate C78a, 1.25 g, 2.64 mmol) in acetonitrile (10 mL) was added NIS (0.66 g, 2.78 mmol) and the reaction mixture was stirred at RT for 30 min. Water was added and the mixture was extracted with EtOAc (×2). The combined organic extracts were dried (Na₂SO₄), filtered and concentrated. The crude residue was purified by normal phase chromatography (eluent: (CH₂Cl₂/10% MeOH) in CH₂Cl₂, 0 to 30%) to give the title compound. UPLC-MS-4: Rt=0.97 min; MS m/z [M+H]⁺ 600.4.

### Step 2: Tert-butyl 6-(4-(5-chloro-6-methyl-1-(tetrahydro-2H-pyran-2-yl)-1H-indazol-4-yl)-3-(4-(3-methoxyazetidin-1-yl)-2,2-dimethylpiperidin-1-yl)-5-methyl-1H-pyrazol-1-yl)-2-azaspiro[3.3]heptane-2-carboxylate

To a mixture of tert-butyl 6-(4-iodo-3-(4-(3-methoxyazetidin-1-yl)-2,2-dimethylpiperidin-1-yl)-5-methyl-1H-pyrazol-1-yl)-2-azaspiro[3.3]heptane-2-carboxylate (Step 1, 1.23 g, 2.05 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 1.00 g, 2.67 mmol), RuPhos (96.0 mg, 0.21 mmol) and RuPhos-Pd-G3 (137 mg, 0.16 mmol) in dioxane (10 mL) was added K₃PO₄ (2 M in water, 3.80 mL, 6.15 mmol) and the reaction mixture was stirred at 95° C. for 1 h under nitrogen atmosphere. The reaction mixture was poured into water and extracted with EtOAc (×2). The combined organic extracts were dried (Na₂SO₄) and concentrated. The crude residue was diluted with THF (2 mL), SiliaMetS®Thiol (0.15 mmol) was added and the mixture swirled for 1 h at 40° C. The mixture was filtered, the filtrate was concentrated and the crude residue was purified by normal phase chromatography (eluent: (CH₂Cl₂/10% MeOH) in CH₂Cl₂, 0 to 35%) to give the title compound. UPLC-MS-4: Rt=1.17 min; MS m/z [M+H]⁺ 722.5/724.5.

### Step 3: 5-Chloro-4-(3-(4-(3-methoxyazetidin-1-yl)-2,2-dimethylpiperidin-1-yl)-5-methyl-1-(2-azaspiro[3.3]heptan-6-yl)-1H-pyrazol-4-yl)-6-methyl-1H-indazole

To a solution of tert-butyl 6-(4-(5-chloro-6-methyl-1-(tetrahydro-2H-pyran-2-yl)-1H-indazol-4-yl)-3-(4-(3-methoxyazetidin-1-yl)-2,2-dimethylpiperidin-1-yl)-5-methyl-1H-pyrazol-1-yl)-2-azaspiro[3.3]heptane-2-carboxylate (Step 2, 1.17 g, 1.62 mmol) in CH₂Cl₂ (5 mL) was added under argon TFA (1.25 mL, 16.2 mmol) and the reaction mixture was stirred at RT for 1 h. The reaction mixture was neutralized by addition of a sat. aq. NaHCO₃ solution and extracted with EtOAc, then with nbuOH. The combined organic extracts were washed with water, dried (Na₂S₂O₄) and concentrated to dryness. The compound was used without purification in the next step. UPLC-MS-4: Rt=0.43/0.48 min; MS m/z [M+H]⁺ 538.4/540.3.

### Step 4: 1-(6-(4-(5-Chloro-6-methyl-1H-indazol-4-yl)-3-(4-(3-methoxyazetidin-1-yl)-2,2-dimethylpiperidin-1-yl)-5-methyl-1H-pyrazol-1-yl)-2-azaspiro[3.3]heptan-2-yl)prop-2-en-1-one

To a stirred solution of 5-chloro-4-(3-(4-(3-methoxyazetidin-1-yl)-2,2-dimethylpiperidin-1-yl)-5-methyl-1-(2-azaspiro[3.3]heptan-6-yl)-1H-pyrazol-4-yl)-6-methyl-1H-indazole (Step 3, 1.62 mmol) in THF (6 mL) at 0° C. was added NaHCO₃ (0.5M in water, 19.5 mL, 9.73 mmol) and acryloyl chloride (0.20 mL, 2.43 mmol). The reacture mixture was stirred at 0° C. for 15 min. The reaction mixture was diluted with EtOAc and extracted with water (×2). The organic layers was dried (Na₂SO₄) and concentrated. The isomers were separated by chiral SFC (C-SFC-4: mobile phase: CO₂/[IPA+0.1% NH₃]: 65/35). The purified fractions were concentrated, dioxane was added and the material was lyophilized to give the title compound Example 202b as the second eluting peak (white powder): ¹H NMR (400 MHz, DMSO-d₆) δ 12.95 (s, 1H), 7.53 (s, 1H), 7.44 (s, 1H), 6.30 (m, 1H), 6.10 (m, 1H), 5.67 (m, 1H), 4.73 (m, 1H), 4.33 (s, 1H), 4.28 (s, 1H), 4.05 (s, 1H), 3.99 (s, 1H), 3.40-3.32 (m, 2H), 3.10 (s, 3H), 3.05 (m, 1H), 2.81 (m, 1H), 2.74-2.58 (m, 6H), 2.47 (s, 3H), 2.14 (m, 1H), 1.99 (s, 3H), 1.45 (m, 1H), 1.30 (m, 1H), 1.11 (s, 3H), 0.84-0.70 (m, 4H), 0.64 (m, 1H); UPLC-MS-4: Rt=0.79 min; MS m/z [M+H]⁺ 592.5/594.5; C-SFC-3 (mobile phase: CO₂/[IPA+0.1% Et₃N]: 65/35): Rt=1.20 min. The other isomer Example 202a was obtained as the first eluting peak: C-SFC-3 (mobile phase: CO₂/[IPA+0.1% Et₃N]: 65/35): Rt=0.82 min.

Method-11a: similar to Method-11 except that in Step 1 NBS was used instead of NIS to give the corresponding 4-bromo analog.

Method-11b: similar to Method-11 except that in Step 3, after completion of the reaction, the reaction mixture was concentrated and the resulting trifluoroacetate salt was used directly in the next step as described in Step 2 Method-8.

Method-11c: similar to Method-11 except that Step 4 was performed using E₃N acrylic acid and T₃P in CH₂Cl₂ as described in Step 3 in Method-7.

Method-11d: similar to Method-11 except that in Step 2 toluene was used as a solvent instead of dioxane.

The following examples 203a to 121 b were prepared using analogous methods to Method-11 from intermediates (in Step 1, 2 or 3) described in the intermediates synthesis section.

In Step 4, If observed the side product resulting from reaction of the acryloyl chloride with the indazole NH was hydrolyzed by treatment with LiOH as described in Method-5 or in Method-12.

When the final separation of isomers is performed by preparative HPLC using acidic conditions the purified fractions were extracted with a sat. aq. NaHCO₃ solution before lyophilization from CH₃CN/H₂O to give the title compound as a free base.

| | | Method, | |
|---|---|---|---|
| | | intermediates | |
| | | (Step 1) and | |
| | | achiral or chiral | |
| | | separation | |
| | | conditions | |
| | | used and order | |
| Example | Structure | of elution | Characterizing data |
| 203a/ 203b | 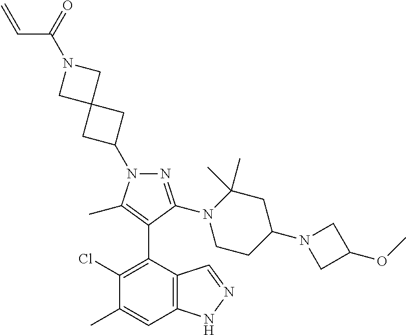 [SMILES: C=CC(=O)N1CC2(CC(n3nc(N4CCC(N5CC(OC)C5)CC4(C)C)c(-c4c(Cl)c(C)cc5[nH]ncc45)c3C)C2)C1] 1-(6-(4-(5-Chloro-6-methyl- 1H-indazol-4-yl)-3-(4-(3- methoxyazetidin-1-yl)-2,2- dimethylpiperidin-1-yl)-5- methyl-1H-pyrazol-1-yl)-2- azaspiro[3.3]heptan-2- yl)prop-2-en-1-one | Using Method- 11a from Intermediate C78b (Step 1) and Intermediate D1 (Step 2) and RP- HPLC-1 (mobile phase: CH₃CN in H₂O + 5% TFA, 5 to 60%); Example 203a = 1st eluting isomer, Example 203b = 2nd eluting isomer | Example 203a: UPLC-MS-2a: Rt = 0.77 min; MS m/z [M + H]⁺ 592.2/593.8. Example 203b: UPLC-MS-2a: Rt = 0.80 min. |
| 204a/ 204b | 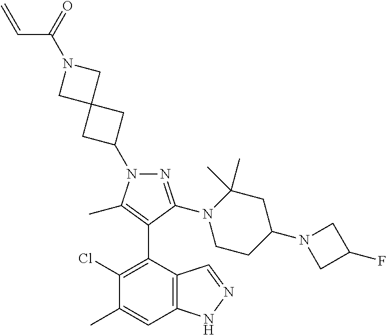 [SMILES: C=CC(=O)N1CC2(CC(n3nc(N4CCC(N5CC(F)C5)CC4(C)C)c(-c4c(Cl)c(C)cc5[nH]ncc45)c3C)C2)C1] 1-(6-(4-(5-chloro-6-methyl- 1H-indazol-4-yl)-3-(4-(3- fluoroazetidin-1-yl)-2,2- dimethylpiperidin-1-yl)-5- methyl-1H-pyrazol-1-yl)-2- azaspiro[3.3]heptan-2- yl)prop-2-en-1-one | Using Method- 11a from Intermediate C79a (Step 1) and Intermediate D1 (Step 2) and RP- HPLC-1 (mobile phase: CH₃CN in H₂O + 5% TFA, 5 to 50%); Example 204a = 1st eluting isomer, Example 204b = 2nd eluting isomer | Example 204b: ¹H NMR (400 MHz, DMSO-d₆) δ 12.95 (s, 1H), 7.53 (s, 1H), 7.44 (s, 1H), 6.30 (m, 1H), 6.10 (m, 1H), 5.67 (m, 1H), 5.02 (m, 1H), 4.74 (m, 1H), 4.33 (s, 1H), 4.28 (s, 1H), 4.05 (s, 1H), 3.99 (s, 1H), 3.46-3.36 (m, 2H), 3.06 (m, 1H), 2.99-2.78 (m, 3H), 2.75-2.66 (m, 4H), 2.47 (s, 3H), 2.21 (m, 1H), 1.99 (s, 3H), 1.45 (m, 1H), 1.33-1.23 (m, 1H), 1.12 (s, 3H), 0.76 (s, 1.5H), 0.72 (s, 1.5H), 0.70 (m, 1H), 0.66 (m, 1H). UPLC-MS- 4: Rt = 0.73 min; MS m/z [M + H]⁺ 580.5/582.5. Example 204a: UPLC-MS-4: Rt = 0.69 min. |
| 205a/ 205b | 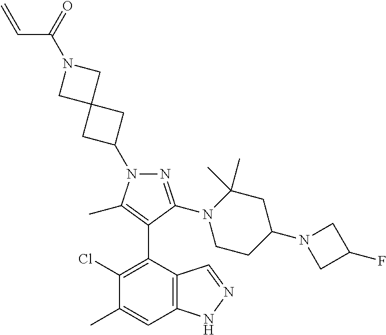 [SMILES: C=CC(=O)N1CC2(CC(n3nc(N4CCC(N5CC(F)C5)CC4(C)C)c(-c4c(Cl)c(C)cc5[nH]ncc45)c3C)C2)C1] 1-(6-(4-(5-chloro-6-methyl- 1H-indazol-4-yl)-3-(4-(3- fluoroazetidin-1-yl)-2,2- dimethylpiperidin-1-yl)-5- methyl-1H-pyrazol-1-yl)-2- azaspiro[3.3]heptan-2- yl)prop-2-en-1-one | Using Method- 11a from Intermediate C79b (Step 1) and Intermediate D1 (Step 2) and RP- HPLC-1 (CH₃CN in H₂O + 5% TFA, 5 to 50%); Example 205a = 1st eluting isomer, Example 205b = 2nd eluting isomer | Example 205a: UPLC-MS-4: Rt = 0.69 min; MS m/z [M + H]⁺ 580.5/582.5. Example 205b: UPLC-MS-4: Rt = 0.73 min. |
| 206a/ 206b | 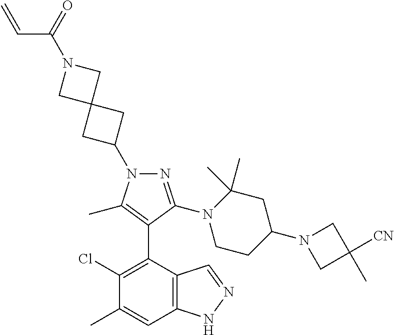 [SMILES: C=CC(=O)N1CC2(CC(n3nc(N4CCC(N5CC(C)(C#N)C5)CC4(C)C)c(-c4c(Cl)c(C)cc5[nH]ncc45)c3C)C2)C1] 1-(1-(1-(2-acryloyl-2- azaspiro[3.3]heptan-6-yl)-4- (5-chloro-6-methyl-1H- indazol-4-yl)-5-methyl-1H- pyrazol-3-yl)-2,2- dimethylpiperidin-4-yl)-3- methylazetidine-3-carbonitrile | Using Method- 11a from Intermediate C80a (Step 1) and Intermediate D1 (Step 2) and RP- HPLC-1 (mobile phase: CH₃CN in H₂O + 5% TFA, 5 to 50%); Example 206a = 1st eluting isomer, Example 206b = 2nd eluting isomer | Example 206b: ¹H NMR (400 MHz, DMSO-d₆) δ 12.96 (s, 1H), 7.53 (s, 1H), 7.44 (s, 1H), 6.30 (m, 1H), 6.10 (m, 1H), 5.67 (m, 1H), 4.74 (m, 1H), 4.33 (s, 1H), 4.28 (s, 1H), 4.05 (s, 1H), 4.00 (s, 1H), 3.30-3.27 (m, 2H), 3.06 (m, 1H), 3.01- 2.94 (m, 2H), 2.82 (m, 1H), 2.75-2.65 (m, 4H), 2.47 (s, 3H), 2.26 (m, 1H), 1.99 (s, 3H), 1.47 (s, 3H), 1.49-1.40 (m, 1H), 1.28-1.22 (m, 1H), 1.11 (s, 3H), 0.76 (s, 1.5H), 0.74 (s, 1.5H), 0.80-0.70 (m, 1H), 0.67-0.57 (m, 1H). UPLC- MS-4: Rt = 0.78 min; MS m/z [M + H]⁺ 601.5/603.5. Example 206a: UPLC-MS-4: Rt = 0.76 min. |
| 207a/ 207b | 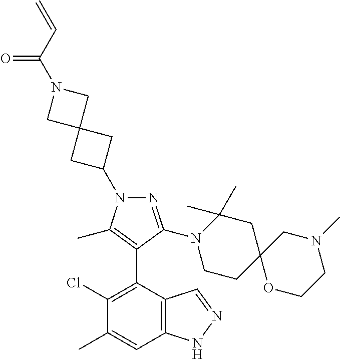 [SMILES: C=CC(=O)N1CC2(CC(n3nc(N4CCC5(CN(C)CCO5)CC4(C)C)c(-c4c(Cl)c(C)cc5[nH]ncc45)c3C)C2)C1] 1-(6-(4-(5-chloro-6-methyl- 1H-indazol-4-yl)-5-methyl-3- (4,8,8-trimethyl-1-oxa-4,9- diazaspiro[5.5]undecan-9-yl)- 1H-pyrazol-1-yl)-2- azaspiro[3.3]heptan-2- yl)prop-2-en-1-one | Using Method- 11b from Intermediate C27a (Step 2) and Intermediate D1 (Step 2) and C- SFC-1 (mobile phase: 25:75 [IPA + 0.1% Et₃N]/CO₂); Example 207a = 1st eluting isomer, Example 207b = 2nd eluting isomer | Example 207b: ¹H NMR (600 MHz, DMSO-d₆) δ 12.94 (s, 1H), 7.54 (s, 1H), 7.43 (s, 1H), 6.29 (m, 1H), 6.09 (m, 1H), 5.67 (m, 1H), 4.74 (m, 1H), 4.33 (s, 1H), 4.28 (s, 1H), 4.05 (s, 1H), 3.99 (s, 1H), 3.49-3.61 (m, 2H), 2.64-2.77 (m, 5H), 2.47 (s, 3H), 2.23 (m, 1H), 2.08 (m, 1H), 2.02 (s, 3H), 1.99 (s, 3H), 1.97 (m, 1H), 1.77-1.87 (m, 2H), 1.55 (m, 1H), 1.23 (m, 1H), 1.19 (s, 3H), 1.11 (m, 1H), 0.87 (m, 1H), 0.69-0.76 (m, 3H). UPLC- MS-3: Rt = 0.73 min, MS m/z [M + H]⁺ 592.5/594.5; C-SFC- 3 (mobile phase: 25:75 [IPA + 0.025% NH₃]/CO₂): Rt = 3.33 min. Example 207a: C- SFC-3 (mobile phase: 25:75 [IPA + 0.025% NH₃]/CO₂): Rt = 2.56 min. |
| 208a/ 208b | 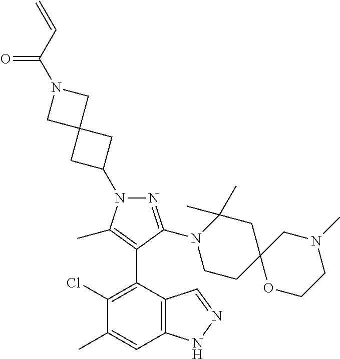 [SMILES: C=CC(=O)N1CC2(CC(n3nc(N4CCC5(CN(C)CCO5)CC4(C)C)c(-c4c(Cl)c(C)cc5[nH]ncc45)c3C)C2)C1] 1-(6-(4-(5-chloro-6-methyl- 1H-indazol-4-yl)-5-methyl-3- (4,8,8-trimethyl-1-oxa-4,9- diazaspiro[5.5]undecan-9-yl)- 1H-pyrazol-1-yl)-2- azaspiro[3.3]heptan-2- yl)prop-2-en-1-one | Using Method- 11b from Intermediate C27b (Step 2) and Intermediate D1 (Step 2) and C- SFC-5 (mobile phase: 25:75 [MeOH + 0.1% Et₃N]/CO₂); Example 208a = 1st eluting isomer, Example 208b = 2nd eluting isomer | Example 208a: UPLC-MS-2a: Rt = 0.80 min, MS m/z [M + H]⁺ 592.5/594.5; C-SFC-6 (mobile phase: 25:75 [MeOH + 0.1% Et₃N]/CO₂): Rt = 2.92 min. Example 208b: C- SFC-6 (mobile phase: 25:75 [MeOH + 0.1% Et₃N]/CO₂): Rt = 3.40 min. |
| 209a/ 209b | 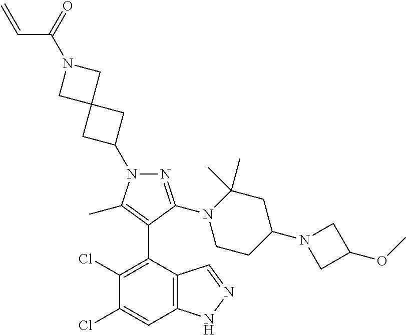 [SMILES: C=CC(=O)N1CC2(CC(n3nc(N4CCC(N5CC(OC)C5)CC4(C)C)c(-c4c(Cl)c(Cl)cc5[nH]ncc45)c3C)C2)C1] 1-(6-(4-(5,6-dichloro-1H- indazol-4-yl)-3-(4-(3- methoxyazetidin-1-yl)-2,2- dimethylpiperidin-1-yl)-5- methyl-1H-pyrazol-1-yl)-2- azaspiro[3.3]heptan-2- yl)prop-2-en-1-one | Using Method- 11a from Intermediate C78a (Step 1) and D6 (Step 2) and normal phase chormatography (eluent: MeOH/CH₂Cl₂ (90/10) in CH₂Cl₂ 0% to 40%); Example 209a = 1st eluting isomer, Example 209b = 2nd eluting isomer | Example 209b: UPLC-MS-4: Rt = 0.78 min; MS m/z [M + H]⁺ 612.4/614.4/616.4. Example 209a: UPLC-MS-4: Rt = 0.74 min; MS m/z [M + H]⁺ 612.4/614.4/616.4. |
| 210a/ 210b | 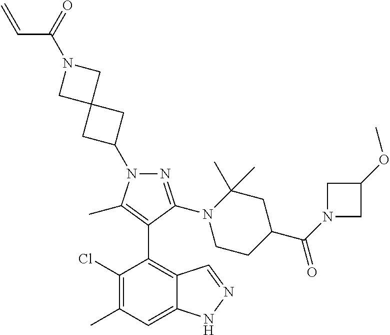 [SMILES: C=CC(=O)N1CC2(CC(n3nc(N4CCC(C(=O)N5CC(OC)C5)CC4(C)C)c(-c4c(Cl)c(C)cc5[nH]ncc45)c3C)C2)C1] 1-(6-(4-(5-chloro-6-methyl- 1H-indazol-4-yl)-3-(4-(3- methoxyazetidine-1- carbonyl)-2,2- dimethylpiperidin-1-yl)-5- methyl-1H-pyrazol-1-yl)-2- azaspiro[3.3]heptan-2- yl)prop-2-en-1-one | Using Method- 11bc from Intermediate C81a (Step 1) and Intermediate D1 (Step 2) and C- SFC-4 (mobile phase: (CO₂/[IPA + 0.1% Et₃N]: 75/35); Example 210a = 1st eluting isomer, Example 210b = 2nd eluting isomer | Example 210b: ¹H NMR (400 MHz, DMSO-d₆) δ 12.97 (s, 1H), 7.53 (s, 1H), 7.44 (s, 1H), 6.36-6.24 (m, 1H), 6.16-6.03 (m, 1H), 5.72-5.61 (m, 1H), 4.82-4.67 (m, 1H), 4.33 (s, 1H), 4.28 (s, 1H), 4.27-4.21 (m, 1H), 4.18-4.10 (m, 1H), 4.05 (s, 1H), 4.00 (s, 1H), 3.98-3.86 (m, 2H), 3.63-3.47 (m, 1H), 3.21-3.16 (m, 1H), 3.18 (s, 3H), 2.88-2.75 (m, 1H), 2.76-2.63 (m, 4H), 2.47 (s, 3H), 1.99 (s, 3H), 1.48-1.33 (m, 1H), 1.28-1.17 (m, 3H), 1.14 (s, 3H), 1.11-0.88 (m, 1H), 0.80-0.68 (m, 3H); UPLC- MS-1a: Rt = 0.97 min; MS m/z [M + H]⁺ 620.5/622.5; C-SFC- 3 (mobile phase: CO₂/IPA 75/35): Rt = 2.00 min, Example 210a: C-SFC-3 (mobile phase: CO₂/IPA 75/35): Rt = 1.24 min. |
| 211a/ 211b | 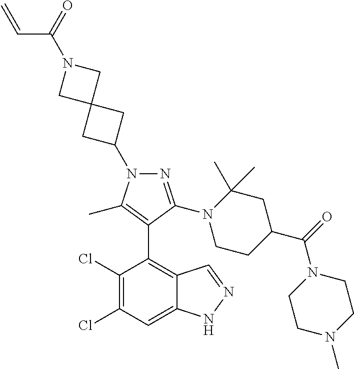 [SMILES: C=CC(=O)N1CC2(CC(n3nc(N4CCC(C(=O)N5CCN(C)CC5)CC4(C)C)c(-c4c(Cl)c(Cl)cc5[nH]ncc45)c3C)C2)C1] 1-(6-(4-(5,6-dichloro-1H- indazol-4-yl)-3-(2,2-dimethyl- 4-(4-methylpiperazine-1- carbonyl)piperidin-1-yl)-5- methyl-1H-pyrazol-1-yl)-2- azaspiro[3.3]heptan-2- yl)prop-2-en-1-one | Using Method- 11bd from Intermediate C91a (Step 1) and D6 (Step 2) and RP-HPLC-5 mobile phase: A: [5 mM ammonium bicarbonate + 0.1% NH₃ in Water]/ B: CH₃CN, gradient: 20 to 45% B in 32 min, 45% for 5 min, 45 to 100% B in 2 min); Example 211a = 1st eluting isomer, Example 211b = 2nd eluting isomer | Example 211b: UPLC-MS-4: Rt = 0.79 min; MS m/z [M + H]⁺ 653.3/655.3/657.3, Example 211a: UPLC-MS-4: Rt = 0.74 min; MS m/z [M + H]⁺ 653.3/655.3/657.3. |
| 212a/ 212b | 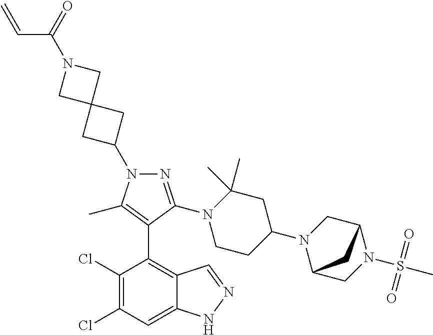 [SMILES: C=CC(=O)N1CC2(CC(n3nc(N4CCC(N5C[C@H]6C[C@@H]5CN6S(C)(=O)=O)CC4(C)C)c(-c4c(Cl)c(Cl)cc5[nH]ncc45)c3C)C2)C1] 1-(6-(4-(5,6-dichloro-1H- indazol-4-yl)-3-(2,2-dimethyl- 4-((1R,4R)-5- (methylsulfonyl)-2,5- diazabicyclo[2.2.1]heptan-2- yl)piperidin-1-yl)-5-methyl- 1H-pyrazol-1-yl)-2- azaspiro[3.3]heptan-2- yl)prop-2-en-1-one | Using Method- 11bc from Intermediate C99a (Step 1) and Intermediate D6 (Step 2) and C- SFC-35 (mobile phase: (CO₂/[IPA + 0.1% Et₃N]: 75/35); Example 212a = 1st eluting isomer, Example 212b = 2nd eluting isomer | Example 212b: UPLC-MS-4: Rt = 0.75 min; MS m/z [M + H]⁺ 701.4/703.3/705.3; C-SFC- 3 (mobile phase: CO₂/[IPA + 0.025% NH₃] 70/30): Rt = 3.35 min, Example 212a: UPLC- MS-4: Rt = 0.71 min; MS m/z [M + H]⁺ 701.2/703.2/705.2; C-SFC-3 (mobile phase: CO₂/[IPA + 0.025% NH₃] 70/30): Rt = 2.20 min. |

### Method-12 for the Preparation of Example 213a and 213b: (R)-1-(6-(4-(5,6-Dichloro-1H-indazol-4-yl)-3-(2,2-dimethyl-4-(morpholinomethyl)piperidin-1-yl)-5-methyl-1H-pyrazol-1-yl)-2-azaspiro[3.3]heptan-2-yl)prop-2-en-1-one Isomer 1 and Isomer 2

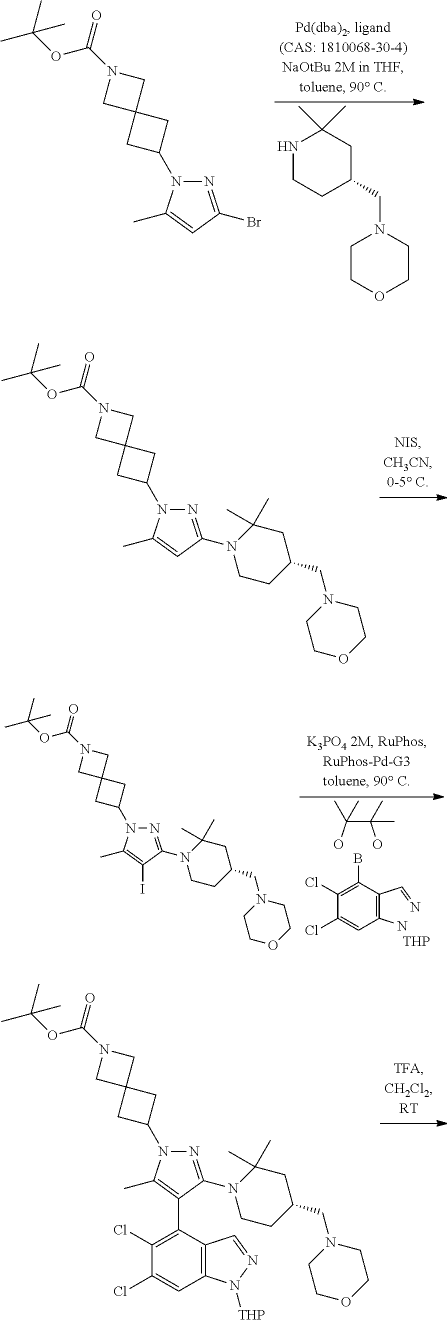

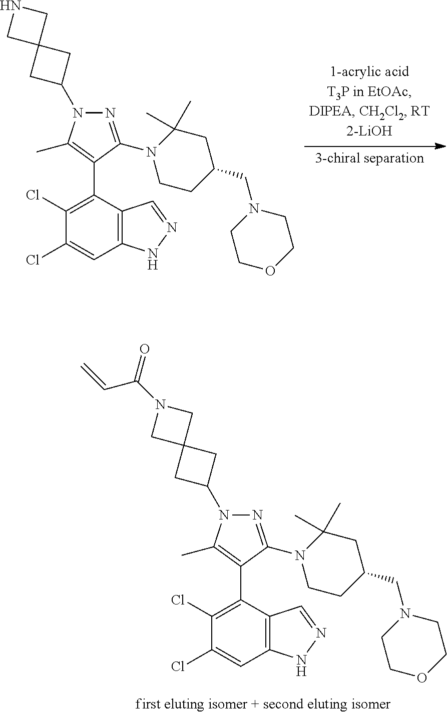 [SMILES: Cc1c(-c2c(Cl)c(Cl)cc3[nH]ncc23)c(N2CC[C@@H](CN3CCOCC3)CC2(C)C)nn1C1CC2(CNC2)C1 ; C=CC(=O)N1CC2(CC(n3nc(N4CC[C@@H](CN5CCOCC5)CC4(C)C)c(-c4c(Cl)c(Cl)cc5[nH]ncc45)c3C)C2)C1]

### Step 1: Tert-butyl (R)-6-(3-(2,2-dimethyl-4-(morpholinomethyl)piperidin-1-yl)-5-methyl-1H-pyrazol-1-yl)-2-azaspiro[3.3]heptane-2-carboxylate

In an ace tube, tert-butyl 6-(3-bromo-5-methyl-1H-pyrazol-1-yl)-2-azaspiro[3.3]heptane-2-carboxylate (Intermediate C1, 6.00 g, 16.8 mmol), (R)-4-((2,2-dimethylpiperidin-4-yl)methyl)morpholine (Intermediate A70, 4.29 g, 20.2 mmol), Pd(dba)₂ (968 mg, 1.68 mmol) and bis(3,5-bis(trifluoromethyl)phenyl)(2′,6′-bis(dimethylamino)-3,6-dimethoxybiphenyl-2-yl)phosphine (CAS: 1810068-30-4, 1.02 g, 1.35 mmol) were suspended in toluene (75 mL). NaOtBu (2 M in THF, 25.3 mL, 55.0 mmol) was added, the vial was flushed with N₂ and the reaction mixture was placed in a pre-heated bath at 90° C. and stirred for 16 h. Then the RM was poured into a sat. aq. NaHCO₃ solution and extracted with EtOAc (×3). The combined organic layers were dried (Na₂SO₄), filtered and concentrated under reduced pressure and the crude residue was purified by normal phase chromatography (eluent: MeOH in CH₂Cl₂ 0 to 10%) to give the title compound as a brown foam. UPLC-MS-4: Rt=0.62 min; MS m/z [M+H]⁺ 488.5.

Alternatively Tert-butyl (R)-6-(3-(2,2-dimethyl-4-(morpholinomethyl)piperidin-1-yl)-5-methyl-1H-pyrazol-1-yl)-2-azaspiro[3.3]heptane-2-carboxylate can be prepare as described for C116.

### Step 2: Tert-butyl (R)-6-(3-(2,2-dimethyl-4-(morpholinomethyl)piperidin-1-yl)-4-iodo-5-methyl-1H-pyrazol-1-yl)-2-azaspiro[3.3]heptane-2-carboxylate

To an ice-cooled solution of tert-butyl (R)-6-(3-(2,2-dimethyl-4-(morpholinomethyl)piperidin-1-yl)-5-methyl-1H-pyrazol-1-yl)-2-azaspiro[3.3]heptane-2-carboxylate (Step 1, 930 mg, 1.91 mmol) in CH₃CN (20 mL) was added NIS (429 mg, 1.91 mmol) and the mixture was stirred under N₂ atmosphere at 0° C. After completion (1 h), the reaction mixture was poured into 10% Na₂S₂O₃ aq. solution and extracted with EtOAc (×2). The combined organic layers were washed with an aq. sat. NaHCO₃ solution, dried (Na₂SO₄), filtered and concentrated to give the title product which was used without further purification in the next step. UPLC-MS-4: Rt=0.99 min; MS m/z [M+H]⁺ 614.5.

### Step 3: Tert-butyl 6-(4-(5,6-dichloro-1-(tetrahydro-2H-pyran-2-yl)-1H-indazol-4-yl)-3-((R)-2,2-dimethyl-4-(morpholinomethyl)piperidin-1-yl)-5-methyl-1H-pyrazol-1-yl)-2-azaspiro[3.3]heptane-2-carboxylate

To a mixture of tert-butyl (R)-6-(3-(2,2-dimethyl-4-(morpholinomethyl)piperidin-1-yl)-4-iodo-5-methyl-1H-pyrazol-1-yl)-2-azaspiro[3.3]heptane-2-carboxylate (Step 2, 1.05 g, 1.71 mmol), 5,6-dichloro-1-(tetrahydro-2H-pyran-2-yl)-4-(4,4,5,5-tetramethyl-1,3,2-dioxaborolan-2-yl)-1H-indazole (Intermediate D6, 815 mg, 2.05 mmol), RuPhos (80.0 mg, 0.17 mmol) and RuPhos-Pd-G3 (143 mg, 0.17 mmol) in toluene (20 mL) was added K₃PO₄ (2 M in water, 2.57 mL, 5.13 mmol) and the reaction mixture was stirred at 90° C. for 2 h under nitrogen atmosphere. The reaction mixture was poured into a sat. aq. NaHCO₃ solution and extracted with EtOAc (×3). The combined organic extracts were dried (Na₂SO₄), filtered and concentrated. The crude residue was diluted with THF (100 mL), SiliaMetS®Thiol (21.3 mmol) was added and the mixture swirled for 1 h at 40° C. The mixture was filtered, the filtrate was concentrated and the crude residue was purified by normal phase chromatography (eluent: EtOAc in n-heptane 0 to 100%) to give the title compound as a beige foam. UPLC-MS-2a: Rt=1.19 min; MS m/z [M+H]⁺ 756.6/758.6/760.6.

### Step 4: (R)-4-((1-(4-(5,6-dichloro-1H-indazol-4-yl)-5-methyl-1-(2-azaspiro[3.3]heptan-6-yl)-1H-pyrazol-3-yl)-2,2-dimethylpiperidin-4-yl)methyl)morpholine

To a solution of tert-butyl 6-(4-(5,6-dichloro-1-(tetrahydro-2H-pyran-2-yl)-1H-indazol-4-yl)-3-((R)-2,2-dimethyl-4-(morpholinomethyl)piperidin-1-yl)-5-methyl-1H-pyrazol-1-yl)-2-azaspiro[3.3]heptane-2-carboxylate (Step 3, 4.94 g, 6.20 mmol) in CH₂Cl₂ (50 mL) was added TFA (14.3 mL, 186 mmol) and the solution was stirred at RT for 16 h. The RM was concentrated, co-evaporated with CH₂Cl₂ (×2) and dried under high vacuum to give the title compound as a trifluoroacetate salt which was used in the next step without further purification. UPLC-MS-4: Rt=0.43 and 0.49 min; MS m/z [M+H]⁺ 572.5/574.5/576.5 .

### Step 5: (R)-1-(6-(4-(5,6-dichloro-1H-indazol-4-yl)-3-(2,2-dimethyl-4-(morpholinomethyl)piperidin-1-yl)-5-methyl-1H-pyrazol-1-yl)-2-azaspiro[3.3]heptan-2-yl)prop-2-en-1-one

A mixture of acrylic acid (0.65 mL, 9.43 mmol), propylphosphonic anhydride (50% in EtOAc, 5.56 mL, 9.43 mmol) and DIPEA (17.6 mL, 101 mmol) in CH₂Cl₂ (80 mL) was stirred for 15 min and then added under a nitrogen atmosphere to a solution of (R)-4-((1-(4-(5,6-dichloro-1H-indazol-4-yl)-5-methyl-1-(2-azaspiro[3.3]heptan-6-yl)-1H-pyrazol-3-yl)-2,2-dimethylpiperidin-4-yl)methyl)morpholine (Step 4, 6.29 mmol) in CH₂Cl₂ (40 mL). The reaction mixture was stirred at RT for 30 min. After completion of the reaction, the reaction mixture was poured into a sat. aq. NaHCO₃ solution and extracted with CH₂Cl₂ (×2). The combined organic extracts were dried (phase separator) and evaporated. The crude residue was diluted in THF, LiOH (2 M, 31.4 mL, 62.9 mmol) was added and the mixture was vigorously stirred at RT for 1 h. EtOAc was added and the layers were separated. The aqueous layer was back-extracted with EtOAc, the combined organic extracts were dried (phase separator) and evaporated. The crude residue was purified by normal phase chromatography (eluent: MeOH in CH₂Cl₂ 0 to 10%) to give the title compound. The isomers were separated by chiral SFC (C-SFC-4; mobile phase: CO₂/[IPA+0.1% Et₃N] 72/28) to give after lyophilization (CH₃CN/water) the title compound Example 213b as the second eluting peak (white solid): ¹H NMR (400 MHz, DMSO-d₆) δ 13.2 (s, 1H), 7.78 (s, 1H), 7.63 (s, 1H), 6.30 (m, 1H), 6.09 (m, 1H), 5.67 (m, 1H), 4.75 (m, 1H), 4.32 (s, 1H), 4.28 (s, 1H), 4.05 (s, 1H), 4.00 (s, 1H), 3.51 (m, 4H), 3.23-3.12 (m, 1H), 2.84-2.65 (m, 5H), 2.28-2.19 (m, 4H), 2.00 (s, 3H), 1.92 (m, 2H), 1.78 (m, 1H), 1.54 (m, 1H), 1.35 (m, 1H), 1.12 (s, 3H), 0.77-0.56 (m, 5H). UPLC-MS-4: Rt=0.78 min; MS m/z [M+H]⁺ 626.6/628.6/630.6. C-SFC-3 (mobile phase: CO₂/[IPA+0.025% NH₃]: 75/25): Rt=2.40 min. The other isomer Example 213a was obtained as the first eluting peak: C-SFC-3 (mobile phase: CO₂/[IPA+0.025% NH₃]: 75/25): Rt=1.52 min.

Method-12a: similar to Method-12 except that Step 5 was performed using acryloyl chloride and NaHCO₃ as described in Method-8 Step 3.

Method-12b: similar to Method-12 except that Step 2 was performed in THF instead of CH₃CN.

Method-12c: similar to Method-12 except that Step 3 was performed using chloro(crotyl)(tri-tert-butylphosphine)Pd(II) (CAS [1334497-00-5]) as a catalyst in dioxane as solvent instead of RuPhos, RuPhos-Pd-G3 in toluene.

The following examples 214a to 242b were prepared using analogous methods to Method-12 from intermediates (in Step 1, 2 and 3) described in the intermediates synthesis section or commercially available.

When the final separation of isomers is performed by preparative HPLC using acidic conditions the purified fractions were extracted with a sat. aq. NaHCO₃ solution before lyophilization from CH₃CN/H₂O to give the title compound as a free base.

| | | Method, | |
|---|---|---|---|
| | | intermediates (Step 1, | |
| | | 2 or 3) and chiral | |
| | | separation conditions | |
| | | used and order of | |
| Example | Structure | elution | Characterizing data |
| 214a/ 214b | 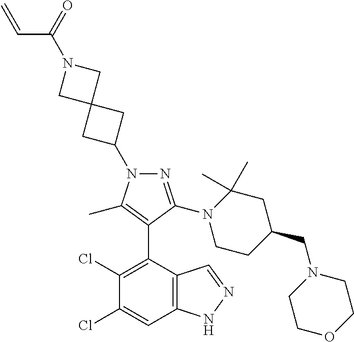 (S)-1-(6-(4-(5,6-dichloro- 1H-indazol-4-yl)-3-(2,2- dimethyl-4- (morpholinomethyl) piperidin-1-yl)-5-methyl-1H- pyrazol-1-yl)-2- azaspiro[3.3]heptan-2- yl)prop-2-en-1-one | Using Method-12 from Intermediate A71 (Step 1), Intermediate D6 (Step 3) and RP-HPLC- 5 (mobile phase: A: [5 mM ammonium bicarbonate + 0.1% NH₃ in Water]/ B: CH₃CN, gradient: 35 to 50% B in 40 min, 50% for 2 min, then 50 to 100% in 2 min) Example 214a = 1st eluting isomer, Example 214b = 2nd eluting isomer. | Example 214a: ¹H NMR (400 MHz, DMSO-d₆) δ 13.2 (s, 1H), 7.81 (s, 1H), 7.71 (s, 1H), 6.34 (m, 1H), 6.13 (d, 1H), 5.70 (d, 1H), 4.78 (m, 1H), 4.35 (s, 1H), 4.29 (s, 1H), 4.07 (s, 1H), 4.01 (s, 1H), 3.52 (m, 4H), 2.93 (m, 1H), 2.78 (m, 4H), 2.65 (m, 1H), 2.23 (m, 4H), 1.99 (s, 3H), 1.91 (m, 2H), 1.76 (m, 1H), 1.45 (d, 1H), 1.37 (m, 1H), 1.29 (s, 3H), 1.07 (s, 1.5H), 1.05 (s, 1.5H), 0.81 (t, 1H), 0.34 (m, 1H). UPLC-MS-14 (mobile phase (5 mM ammonium bicarbonate + 0.1% NH₃ in water/CH₃CN + 0.1% NH₃, gradient: 90/10 at 0.01 min, 50/50 at 10.0 min, 20/80 at 20.0 min, 0/100 at 25 min), Rt = 17.2 min, MS m/z [M + H]⁺ 626.2/628.2/ 630.2, Example 214b: UPLC-MS-14 (mobile phase (5 mM ammonium bicarbonate + 0.1% NH₃ in water/CH₃CN + 0.1% NH₃, gradient: 90/10 at 0.01 min, 50/50 at 10.0 min, 20/80 at 20.0 min, 0/100 at 25 min), Rt = 17.5 min, MS m/z [M + H]⁺ 626.2/628.2/ 630.2. |
| 215a/ 215b | 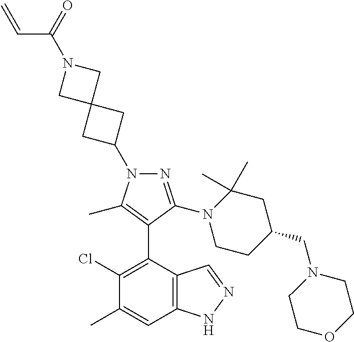 [SMILES: C=CC(=O)N1CC2(CC(n3nc(N4CC[C@@H](CN5CCOCC5)CC4(C)C)c(-c4c(Cl)c(C)cc5[nH]ncc45)c3C)C2)C1] (R)-1-(6-(4-(5-chloro-6- methyl-1H-indazol-4-yl)- 3-(2,2-dimethyl-4- (morpholinomethyl) piperidin-1-yl)-5-methyl-1H- pyrazol-1-yl)-2- azaspiro[3.3]heptan-2- yl)prop-2-en-1-one | Using Method-12 from Intermediate A70 (Step 1), Intermediate D1 (Step 3) and C-SFC-2 (mobile phase: CO₂/[IPA + 0.1% Et₃N] 72/28); Example 215a = 1st eluting isomer, Example 215b = 2nd eluting isomer. | Example 215b: ¹H NMR (400 MHz, DMSO-d₆) δ 12.9 (s, 1H), 7.53 (s, 1H), 7.43 (s, 1H), 6.30 (m, 1H), 6.09 (m, 1H), 5.67 (m, 1H), 4.73 (m, 1H), 4.32 (s, 1H), 4.28 (s, 1H), 4.05 (s, 1H), 4.00 (s, 1H), 3.51 (m, 4H), 3.20-3.10 (m, 1H), 2.82- 2.65 (m, 5H),2.47 (s, 3H), 2.28-2.19 (m, 4H), 1.98 (s, 3H), 1.92 (m, 2H), 1.77 (m, 1H), 1.51 (m, 1H), 1.34 (m, 1H), 1.11 (s, 3H), 0.76-0.58 (m, 5H). UPLC-MS-4: Rt = 0.78 min; MS m/z [M + H]⁺ 606.3/608.3; C-SFC-3 (mobile phase: CO₂/[IPA + 0.025% NH₃] 75/25): Rt = 3.79 min, Example 215a: C-SFC-3 (mobile phase: CO₂/[IPA + 0.025% NH₃] 75/25): Rt = 2.15 min. |
| 216a/ 216b | 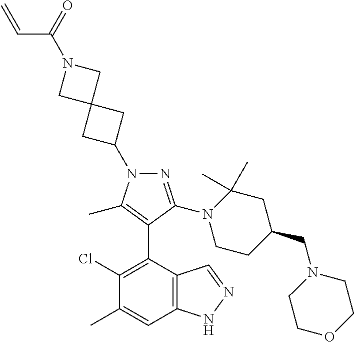 [SMILES: C=CC(=O)N1CC2(CC(n3nc(N4CC[C@H](CN5CCOCC5)CC4(C)C)c(-c4c(Cl)c(C)cc5[nH]ncc45)c3C)C2)C1] (S)-1-(6-(4-(5-chloro-6- methyl-1H-indazol-4-yl)- 3-(2,2-dimethyl-4- (morpholinomethyl) piperidin-1-yl)-5-methyl-1H- pyrazol-1-yl)-2- azaspiro[3.3]heptan-2- yl)prop-2-en-1-one | Using Method-12 from Intermediate A71 (Step 1), Intermediate D1 (Step 3) and RP-HPLC- 5 (mobile phase: A: [5 mM ammonium bicarbonate + 0.1% NH₃ in Water]/ B: CH₃CN, gradient: 35 to 43% B in 20 min, 43% for 19 min, then 50 to 100% in 3 min); Example 216a = 1st eluting isomer, Example 216b = 2nd eluting isomer. | Example 216a: ¹H NMR (600 MHz, DMSO-d₆) δ 12.9 (s, 1H), 7.68 (s, 1H), 7.44 (s, 1H), 6.39 (m, 1H), 6.28 (d, 1H), 5.78 (d, 1H), 4.78 (m, 1H), 4.45 (s, 1H), 4.41 (s, 1H), 4.21 (s, 1H), 4.17 (s, 1H), 3.65 (m, 4H), 2.98-2.81 (m, 6H), 2.67 (s, 3H), 2.35 (m, 4H), 2.05 (m, 5H), 1.83 (m, 1H), 1.47 (m, 1H), 1.36 (m, 4H), 1.15 (s, 3H), 0.96 (m, 1H), 0.55 (m, 1H). UPLC-MS-5: Rt = 1.50 min, MS m/z [M + H]⁺ 606.8/ 608.8; RP-HPLC-8: Rt = 6.51 min, Example 216b: RP-HPLC-8: Rt = 6.60 min. |
| 217a/ 217b | 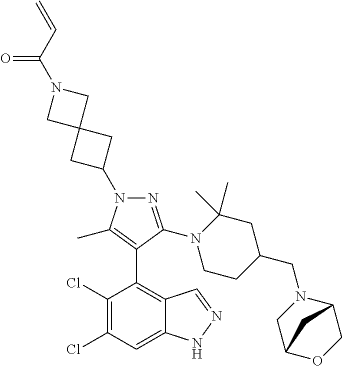 [SMILES: C=CC(=O)N1CC2(CC(n3nc(N4CCC(CN5C[C@@H]6C[C@H]5CO6)CC4(C)C)c(-c4c(Cl)c(Cl)cc5[nH]ncc45)c3C)C2)C1] 1-(6-(3-(4-(((1S,4S)-2- oxa-5- azabicyclo[2.2.1]heptan- 5-yl)methyl)-2,2- dimethylpiperidin-1-yl)-4- (5,6-dichloro-1H-indazol- 4-yl)-5-methyl-1H- pyrazol-1-yl)-2- azaspiro[3.3]heptan-2- yl)prop-2-en-1-one | Using Method-12 from Intermediate C86a (Step 2), Intermediate D6 (Step 3) and RP- HPLC-5 (mobile phase: A: [5 mM ammonium bicarbonate + 0.1% NH₃ in Water]/ B: CH₃CN, gradient: 28 to 42% B in 36 min, 42% for 17 min, then 42 to 100% in 2 min) Example 217a = 1st eluting isomer, Example 217b = 2nd eluting isomer. | Example 217b: ¹H NMR (400 MHz, MeOD) δ 7.74 (s, 1H), 7.68 (s, 1H), 6.38 (m, 1H), 6.29 (d, 1H), 5.78 (d, 1H), 4.78 (m, 1H), 4.45- 4.38 (m, 3H), 4.22 (s, 1H), 4.20 (s, 1H), 4.01 (d, 1H), 3.60 (d, 1H), 3.48 (m, 1H), 3.42 (m, 1H), 2.99-2.82 (m, 6H), 2.51 (d, 1H), 2.39 (m, 1H), 2.28 (m, 1H), 2.11 (s, 3H), 1.88 (d, 1H), 1.85-1.73 (m, 2H), 1.45 (d, 1H), 1.32 (m, 1H), 1.19 (s, 3H), 0.97 (m, 1H), 0.77 (m, 4H). UPLC-MS-10: Rt = 5.33 min, MS m/z [M + H]⁺ 638.2/ 640.2/642.2, Example 217a: UPLC-MS-10: Rt = 5.23 min, MS m/z [M + H]⁺ 638.2/640.2/642.2. |
| 218a/ 218b | 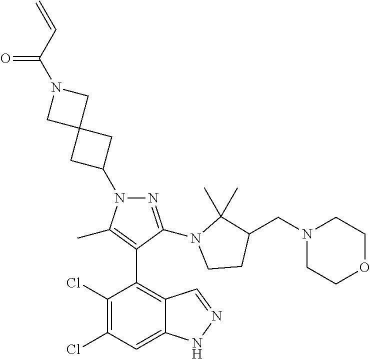 [SMILES: C=CC(=O)N1CC2(CC(n3nc(N4CCC(CN5CCOCC5)C4(C)C)c(-c4c(Cl)c(Cl)cc5[nH]ncc45)c3C)C2)C1] 1-(6-(4-(5,6-dichloro-1H- indazol-4-yl)-3-(2,2- dimethyl-3- (morpholinomethyl) pyrrolidin-1-yl)-5-methyl-1H- pyrazol-1-yl)-2- azaspiro[3.3]heptan-2- yl)prop-2-en-1-one | Using Method-12a from Intermediate C128a (Step 2), Intermediate D6 (Step 3) and RP- HPLC-5 (mobile phase: A: [5 mM ammonium bicarbonate + 0.1% NH₃ in Water]/ B: CH₃CN, gradient: 30 to 50% B in 32 min, 50% for 7 min, then 50% to 100% in 2 min) Example 218a = 1st eluting isomer, Example 218b = 2nd eluting isomer. | Example 218a: UPLC-MS- 14 (mobile phase: A: [5 mM ammonium bicarbonate + 0.1% NH₃ in water]/ B: [CH₃CN + 0.1% NH₃] gradient: 90:10 at 0.01 min, 60:40 at 10.0 min, 30:70 at 20.0 min, 0:100 at 30 min): Rt = 19.7 min, MS m/z [M + H]⁺ = 612.2/614.2/ 616.2, Example 218b: UPLC-MS-14 (mobile phase: A: [5 mM ammonium bicarbonate + 0.1 % NH₃ in water]/ B: [CH₃CN + 0.1% NH₃] gradient: 90:10 at 0.01 min, 60:40 at 10.0 min, 30:70 at 20.0 min, 0:100 at 30 min): Rt = 19.9 min, MS m/z [M + H]⁺ = 612.2/614.2/ 616.2. |
| 219a/ 219b | 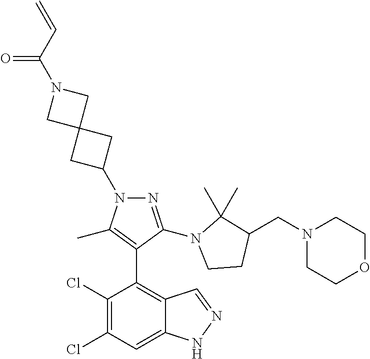 [SMILES: C=CC(=O)N1CC2(CC(n3nc(N4CCC(CN5CCOCC5)C4(C)C)c(-c4c(Cl)c(Cl)cc5[nH]ncc45)c3C)C2)C1] 1-(6-(4-(5,6-dichloro-1H- indazol-4-yl)-3-(2,2- dimethyl-3- (morpholinomethyl) pyrrolidin-1-yl)-5-methyl-1H- pyrazol-1-yl)-2- azaspiro[3.3]heptan-2- yl)prop-2-en-1-one | Using Method-12a from Intermediate C128b (Step 2), Intermediate D6 (Step 3) and RP- HPLC-5 (mobile phase: A: [5 mM ammonium bicarbonate + 0.1% NH₃ in Water]/ B: CH₃CN, gradient: 30 to 50% B in 32 min, 50% for 7 min, then 50% to 100% in 2 min) Example 219a = 1st eluting isomer, Example 219b = 2nd eluting isomer. | Example 219b: UPLC-MS- 14 (mobile phase: A: [5 mM ammonium bicarbonate + 0.1% NH₃ in water]/ B: [CH₃CN + 0.1% NH₃] gradient: 90:10 at 0.01 min, 60:40 at 10.0 min, 30:70 at 20.0 min, 0:100 at 30 min): Rt = 19.9 min, MS m/z [M + H]⁺ = 612.2/614.2/ 616.2, Example 219a: UPLC-MS-14 (mobile phase: A: [5 mM ammonium bicarbonate + 0.1% NH₃ in water]/ B: [CH₃CN + 0.1% NH₃] gradient: 90:10 at 0.01 min, 60:40 at 10.0 min, 30:70 at 20.0 min, 0:100 at 30 min): Rt = 19.7 min, MS m/z [M + H]⁺ = 612.2/614.2/ 616.2. |
| 220a/ 220b | 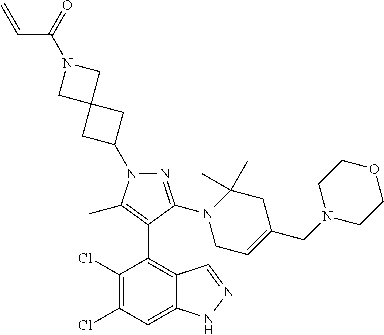 [SMILES: C=CC(=O)N1CC2(CC(n3nc(N4CC=C(CN5CCOCC5)CC4(C)C)c(-c4c(Cl)c(Cl)cc5[nH]ncc45)c3C)C2)C1] 1-(6-(4-(5,6-dichloro-1H- indazol-4-yl)-3-(2,2- dimethyl-4- (morpholinomethyl)-3,6- dihydropyridin-1(2H)-yl)- 5-methyl-1H-pyrazol-1- yl)-2- azaspiro[3.3]heptan-2- yl)prop-2-en-1-one | Using Method-12b from Intermediate A88 (Step 1) and D6 (Step 3) and C-SFC-4 (mobile phase: CO₂/[IPA +0.1% Et₃N] 68/32); Example 220a = 1st eluting isomer, Example 220b = 2nd eluting isomer | Example 220b: UPLC-MS- 4: Rt = 0.77 min; MS m/z [M + H]⁺ 624.6/626.6/ 628.6; C-SFC-3 (mobile phase: CO₂/[IPA + 0.025% NH₃] 68/32): Rt = 2.84 min, Example 220a: UPLC-MS- 4: Rt = 0.76 min; MS m/z [M + H]⁺ 624.4/626.4/ 628.4; C-SFC-3 (mobile phase: CO₂/[IPA + 0.025% NH₃] 68/32): Rt = 1.74 min. |
| 221a/ 221b | 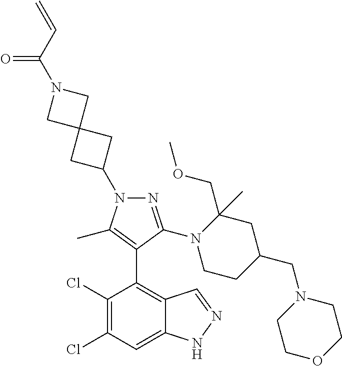 [SMILES: C=CC(=O)N1CC2(CC(n3nc(N4CCC(CN5CCOCC5)CC4(C)COC)c(-c4c(Cl)c(Cl)cc5[nH]ncc45)c3C)C2)C1] 1-(6-(4-(5,6-dichloro-1H- indazol-4-yl)-3-(2- (methoxymethyl)-2- methyl-4- (morpholinomethyl) piperidin-1-yl)-5-methyl-1H- pyrazol-1-yl)-2- azaspiro[3.3]heptan-2- yl)prop-2-en-1-one | Using Method-12 from Intermediate C130a (Step 2), D6 (Step 3) and RP-HPLC-5 (mobile phase (A: [5 mM ammonium bicarbonate + 0.1% NH₃ in Water]/ B: CH₃CN, gradient: 25 to 55% B in 32 min, 55% B for 3 min, 55 to 100% B in 2 min), flow rate: 12 mL/min); Example 221a = 1st eluting isomer, Example 221b = 2nd eluting isomer. | Example 221a: ¹H NMR (400 MHz, MeOD) δ 7.74 (s, 1H), 7.66 (s, 1H), 6.38 (m, 1H), 6.29 (d, 1H), 5.78 (d, 1H), 4.81 (m, 1H), 4.44 (s, 1H), 4.43 (s, 1H), 4.21 (s, 1H), 4.20 (s, 1H), 3.94 (m, 1H), 3.68 (m, 4H), 3.51 (m, 2H), 3.36 (s, 3H), 2.97- 2.83 (m, 5H), 2.38 (m, 4H), 2.08 (s, 3H), 2.06 (m, 2H), 1.85 (m, 2H), 1.60 (m, 1H), 0.93 (m, 1H), 0.79 (s, 1.5H), 0.77 (s, 1.5H), 0.55 (m, 1H); UPLC-MS-15: Rt = 3.10 min, MS m/z [M + H]⁺ 656.4/658.4/660.4, Example 221b: UPLC-MS- 15: Rt = 3.14 min, MS m/z [M + H]⁺ 656.4/658.4/ 660.4. |
| 222a/ 222b | 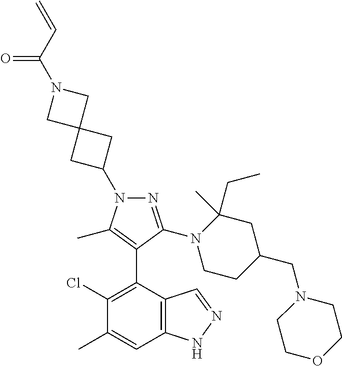 1-(6-(4-(5-chloro-6- methyl-1H-indazol-4-yl)- 3-(2-ethyl-2-methyl-4- (morpholinomethyl) piperidin-1-yl)-5-methyl-1H- pyrazol-1-yl)-2- azaspiro[3.3]heptan-2- yl)prop-2-en-1-one | Using Method-12 from Intermediate C131a (Step 2), Intermediate D1 (Step 3) and RP- HPLC-5 (mobile phase: A: [5 mM ammonium bicarbonate + 0.1% NH₃ in water]/ B: CH₃CN, gradient 45% B for 39 min, 45 to 100% B in 2 min); flow rate: 12 mL/min). Example 222a = 1st eluting isomer, Example 222b = 2nd eluting isomer. | Example 222b: ¹H NMR (400 MHz, MeOD) δ 7.68 (s, 1H), 7.44 (s, 1H), 6.37 (m, 1H), 6.28 (d, 1H), 5.78 (d, 1H), 4.79 (m, 1H), 4.44 (s, 1H), 4.42 (s, 1H), 4.20 (s, 1H), 4.19 (s, 1H), 3.68 (m, 4H), 3.38 (m, 1H), 2.96- 2.80 (m, 6H), 2.54 (s, 3H), 2.38 (m, 4H), 2.06 (m, 5H), 1.85 (m, 2H), 1.71 (m, 1H), 1.62 (m, 2H), 1.05 (m, 1H), 0.73 (m, 6H); UPLC-MS-14 (mobile phase [5 mM ammonium bicarbonate + 0.1% NH₃ in water]/ [CH₃CN + 0.1% NH₃], gradient: 90/10 at 0.01 min, 60/40 at 10 min, 30/70 at 20.0 min, 0/100 at 30 min), Rt = 21.5 min, MS m/z [M + H]⁺ 620.2/622.2/ 624.2, Example 222a: UPLC-MS-14 (mobile phase [5 mM ammonium bicarbonate + 0.1% NH₃ in water]/[CH₃CN + 0.1% NH₃], gradient: 90/10 at 0.01 min, 60/40 at 10 min, 30/70 at 20.0 min, 0/100 at 30 min), Rt = 21.2 min, MS m/z [M + H]⁺ 620.2/622.2/ 624.2. |
| 223 | 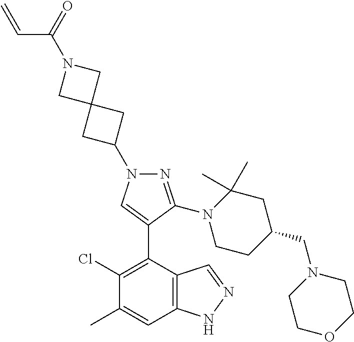 [SMILES: C=CC(=O)N1CC2(CC(n3cc(-c4c(Cl)c(C)cc5[nH]ncc45)c(N4CC[C@@H](CN5CCOCC5)CC4(C)C)n3)C2)C1] (R)-1-(6-(4-(5-chloro-6- methyl-1H-indazol-4-yl)- 3-(2,2-dimethyl-4- (morpholinomethyl) piperidin-1-yl)-1H-pyrazol-1- yl)-2- azaspiro[3.3]heptan-2- yl)prop-2-en-1-one | Using Method-12 from Intermediate C70 (Step 3) and Intermediate D1 (Step 3). | Example 223: UPLC-MS- 4: Rt = 0.67 min; MS m/z [M + H]⁺ 592.4/594.4. |
| 224a/ 224b | 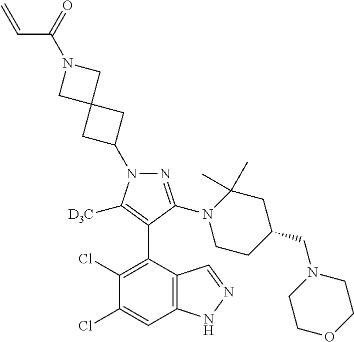 [SMILES: [2H]C([2H])([2H])c1c(-c2c(Cl)c(Cl)cc3[nH]ncc23)c(N2CC[C@@H](CN3CCOCC3)CC2(C)C)nn1C1CC2(C1)CN(C(=O)C=C)C2] (R)-1-(6-(4-(5,6-dichloro- 1H-indazol-4-yl)-3-(2,2- dimethyl-4- (morpholinomethyl)piperidin- 1-yl)-5-(methyl-d₃)- 1H-pyrazol-1-yl)-2- azaspiro[3.3]heptan-2- yl)prop-2-en-1-one | Using Method-12c from Intermediate C71 (Step 3), Intermediate D6 (Step 3) and RP-HPLC- 2 (mobile phase: 5 to 100% CH₃CN in H₂O containing 0.1% TFA); Example 224a = 1st eluting isomer, Example 224b = 2nd eluting isomer. | Example 224b: UPLC-MS- 4: Rt = 0.76 min; MS m/z [M + H]⁺ 629.4/631.4/ 633.4. Example 224a: UPLC-MS- 4: Rt = 0.73 min; MS m/z [M + H]⁺ 629.5/631.5/ 633.5. |
| 225a/ 225b | 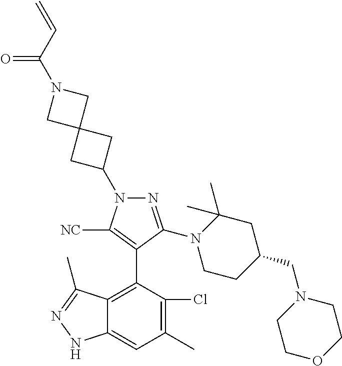 [SMILES: C=CC(=O)N1CC2(CC(n3nc(N4CC[C@@H](CN5CCOCC5)CC4(C)C)c(-c4c(Cl)c(C)cc5[nH]nc(C)c45)c3C#N)C2)C1] (R)-1-(2-acryloyl-2- azaspiro[3.3]heptan-6- yl)-4-(5-chloro-3,6- dimethyl-1H-indazol-4- yl)-3-(2,2-dimethyl-4- (morpholinomethyl)piperidin- 1-yl)-1H-pyrazole-5- carbonitrile | Using Method-12c from Intermediate C72 (Step 3), Intermediate D12 (Step 3) and C-SFC-7 (mobile phase: CO₂/[MeOH + 0.025% NH₃] 55/45); Example 225a = 1st eluting isomer, Example 225b = 2nd eluting isomer. | Example 225a: UPLC-MS- 4: Rt = 0.71 min; MS m/z [M + H]⁺ 631.5/633.5; C- SFC-8 (mobile phase: CO₂/[IPA + 0.025% NH₃] 65/35): Rt = 2.04 min, Example 225b: C-SFC-8 (mobile phase: CO₂/[IPA + 0.025% NH₃] 65/35): Rt = 2.89 min |
| 226a/ 226b | 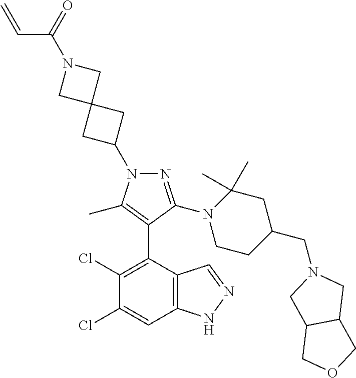 [SMILES: C=CC(=O)N1CC2(CC(n3nc(N4CCC(CN5CC6COCC6C5)CC4(C)C)c(-c4c(Cl)c(Cl)cc5[nH]ncc45)c3C)C2)C1] 1-(6-(4-(5,6-dichloro-1H- indazol-4-yl)-3-(2,2- dimethyl-4-((tetrahydro- 1H-furo[3,4-c]pyrrol- 5(3H)-yl)methyl)piperidin- 1-yl)-5-methyl-1H- pyrazol-1-yl)-2- azaspiro[3.3]heptan-2- yl)prop-2-en-1-one | Using Method-12 from Intermediate C87a (Step 2), Intermediate D6 (Step 3) and RP- HPLC-2 (mobile phase: 15 to 45% CH₃CN in H₂O containing 0.1% TFA); Example 226a = 1st eluting isomer, Example 226b = 2nd eluting isomer. | Example 226a: UPLC-MS- 2e: Rt = 3.58 min; MS m/z [M + H]⁺ 652.3/654.3/ 656.3, Example 226b: UPLC-MS-2e: Rt = 3.88 min; MS m/z [M + H]⁺ 652.3/ 654.3/656.3. |
| 227a/ 227b | 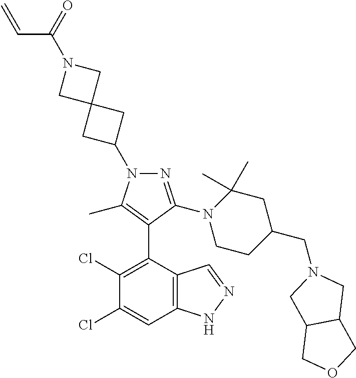 [SMILES: C=CC(=O)N1CC2(CC(n3nc(N4CCC(CN5CC6COCC6C5)CC4(C)C)c(-c4c(Cl)c(Cl)cc5[nH]ncc45)c3C)C2)C1] 1-(6-(4-(5,6-dichloro-1H- indazol-4-yl)-3-(2,2- dimethyl-4-((tetrahydro- 1H-furo[3,4-c]pyrrol- 5(3H)-yl)methyl)piperidin- 1-yl)-5-methyl-1H- pyrazol-1-yl)-2- azaspiro[3.3]heptan-2- yl)prop-2-en-1-one | Using Method-12 from Intermediate C87b (Step 2), Intermediate D6 (Step 3) and RP- HPLC-2 (mobile phase: 15 to 45% CH₃CN in H₂O containing 0.1% TFA); Example 227a = 1st eluting isomer, Example 227b = 2nd eluting isomer. | Example 227b: UPLC-MS- 2e: Rt = 3.83 min; MS m/z [M + H]⁺ 652.3/654.3/ 656.3, Example 227a: UPLC-MS-2e: Rt = 3.54 min; MS m/z [M + H]⁺ 652.3/ 654.3/656.3. |
| 228a/ 228b | 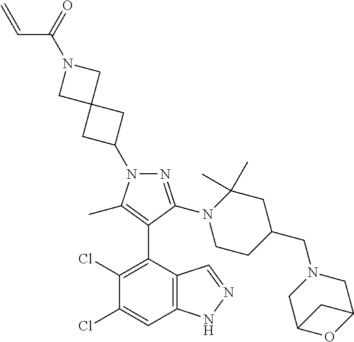 [SMILES: C=CC(=O)N1CC2(CC(n3nc(N4CCC(CN5CC6CC(C5)O6)CC4(C)C)c(-c4c(Cl)c(Cl)cc5[nH]ncc45)c3C)C2)C1] 1-(6-(3-(4-((6-oxa-3- azabicyclo[3.1.1]heptan- 3-yl)methyl)-2,2- dimethylpiperidin-1-yl)-4- (5,6-dichloro-1H-indazol- 4-yl)-5-methyl-1H- pyrazol-1-yl)-2- azaspiro[3.3]heptan-2- yl)prop-2-en-1-one | Using Method-12 from Intermediate A72 (Step 1), Intermediate D6 (Step 3). The mixture of 4 isomers was purified by RP-HPLC-3 (mobile phase: CH₃CN in [H₂O + 0.1% TFA] from 2% to 38%); to give fraction-A (first eluting racemic mixture) and fraction-B (second eluting racemic mixture). The 2 enantiomers in fraction- A were separated using C-SFC-2 (mobile phase: CO₂/IPA + 0.1% Et₃N]: 73/27); Example 228a = 1st eluting isomer, Example 228b = 2nd eluting isomer. | Example 228b: UPLC-MS- 2e: Rt = 3.54 min; MS m/z [M + H]⁺ 638.3/640.3/ 642.3; C-SFC-3 (mobile phase: CO₂/[IPA + 0.025% NH₃] 75/25): Rt = 3.56 min, Example 228a: UPLC-MS- 2e: Rt = 3.59 min; MS m/z [M + H]⁺ 638.3/640.3/ 642.3; C-SFC-3 (mobile phase: CO₂/[IPA + 0.025% NH₃] 75/25): Rt = 2.26 min. |
| 229a/ 229b | 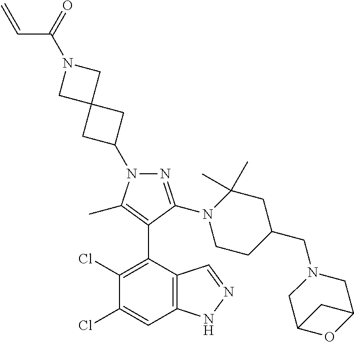 [SMILES: C=CC(=O)N1CC2(CC(n3nc(N4CCC(CN5CC6CC(C5)O6)CC4(C)C)c(-c4c(Cl)c(Cl)cc5[nH]ncc45)c3C)C2)C1] 1-(6-(3-(4-((6-oxa-3- azabicyclo[3.1.1]heptan- 3-yl)methyl)-2,2- dimethylpiperidin-1-yl)-4- (5,6-dichloro-1H-indazol- 4-yl)-5-methyl-1H- pyrazol-1-yl)-2- azaspiro[3.3]heptan-2- yl)prop-2-en-1-one | Using Method-12 from Intermediate A72 (Step 1), Intermediate D6 (Step 3). The mixture of 4 isomers was purified by RP-HPLC-3 (mobile phase: CH₃CN in [H₂O + 0.1% TFA] from 2% to 38%); to give fraction-A (first eluting racemic mixture) and fraction-B (second eluting racemic mixture). The 2 enantiomers in fraction- B were separated using C-SFC-2 (mobile phase: CO₂/IPA + 0.1% Et₃N]: 73/27); Example 229a = 1st eluting isomer, Example 229b = 2nd eluting isomer. | Example 229b: UPLC-MS- 2e: Rt = 3.81 min; MS m/z [M + H]⁺ 638.3/640.3 642.3; C-SFC-3 (mobile phase: CO₂/[IPA + 0.025% NH₃] 75/25): Rt = 3.02 min, Example 229a: UPLC-MS- 2e: Rt = 3.88 min; MS m/z [M + H]⁺ 638.3/640.3/ 642.3; C-SFC-3 (mobile phase: CO₂/[IPA + 0.025% NH₃] 75/25): Rt = 1.97 min. |
| 230a/ 230b | 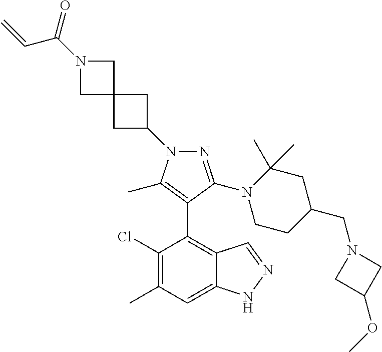 [SMILES: C=CC(=O)N1CC2(CC(n3nc(N4CCC(CN5CC(OC)C5)CC4(C)C)c(-c4c(Cl)c(C)cc5[nH]ncc45)c3C)C2)C1] 1-(6-(4-(5-chloro-6- methyl-1H-indazol-4-yl)- 3-(4-((3-methoxyazetidin- 1-yl)methyl)-2,2- dimethylpiperidin-1-yl)-5- methyl-1H-pyrazol-1-yl)- 2-azaspiro[3.3]heptan-2- yl)prop-2-en-1-one | Using Method-12 from Intermediate C84a (Step 2), Intermediate D1 (Step 3) and C-SFC- 4 (mobile phase: (CO₂/IPA + 0.1% Et₃N]: 75/25); Example 230a = 1st eluting isomer, Example 230b = 2nd eluting isomer | Example 230b: 1H NMR (400 MHz, DMSO-d₆) δ 12.93 (s, 1H), 7.51 (s, 1H), 7.43 (s, 1H), 6.38-6.22 (m, 1H), 6.16-6.04 (m, 1H), 5.74-5.63 (m, 1H), 4.81- 4.67 (m, 1H), 4.33 (s, 1H), 4.28 (s, 1H), 4.05 (s, 1H), 4.00 (s, 1H), 3.92-3.82 (m, 1H), 3.43-3.36 (m, 2H), 3.17-3.11 (m, 1H), 3.10 (s, 3H), 2.81-2.62 (m, 7H), 2.47 (s, 3H), 2.10-2.03 (m, 2H), 1.98 (s, 3H), 1.59-1.39 (m, 2H), 1.33-1.21 (m, 2H), 1.10 (s, 3H), 0.75-0.70 (m, 3H), 0.67-0.57 (m, 1H); UPLC-MS-4: Rt = 0.80 min; MS m/z [M + H]⁺ 606.5/ 608.5; C-SFC-3 (mobile phase: CO₂/[IPA + 0.1% Et₃N] 75/25): Rt = 3.16 min. Example 230a: C-SFC-3 (mobile phase: CO₂/[IPA + 0.1% Et₃N] 75/25): Rt = 2.47 min. |
| 231a/ 231b | 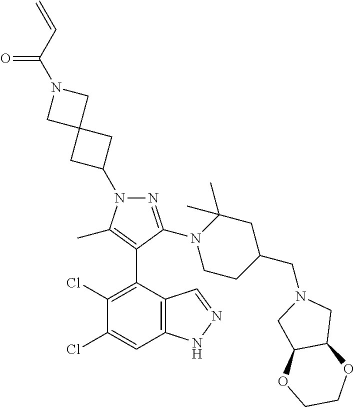 [SMILES: C=CC(=O)N1CC2(CC(n3nc(N4CCC(CN5C[C@@H]6OCCO[C@@H]6C5)CC4(C)C)c(-c4c(Cl)c(Cl)cc5[nH]ncc45)c3C)C2)C1] 1-(6-(4-(5,6-dichloro-1H- indazol-4-yl)-3-(4- (((4aR,7aS)-hexahydro- 6H-[1,4]dioxino[2,3- c]pyrrol-6-yl)methyl)-2,2- dimethylpiperidin-1-yl)-5- methyl-1H-pyrazol-1-yl)- 2-azaspiro[3.3]heptan-2- yl)prop-2-en-1-one | Using Method-12a starting from Intermediate C88a (Step 2), Intermediate D6 (Step 3) and C-SFC- 4 (mobile phase: CO₂/[IPA + 0.1% Et₃N] 70/30); Example 231a = 1st eluting isomer, Example 231b = 2nd eluting isomer. | Example 231b: C-SFC-3 (mobile phase: CO₂/[IPA + 0.025% NH₃] 70/30): Rt = 3.27 min, UPLC-MS-4: Rt = 0.81 min; MS m/z [M + H]⁺ 668.7/670.7/672.7, Example 231a: C-SFC-3 (mobile phase: CO₂/[IPA + 0.025% NH₃] 70/30): Rt = 1.73 min. |
| 232a/ 232b | 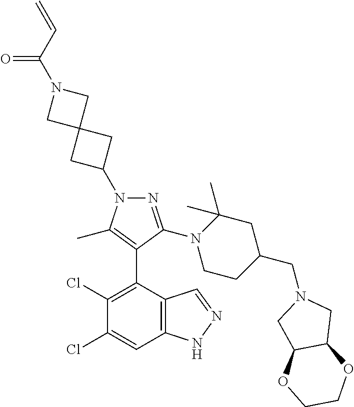 [SMILES: C=CC(=O)N1CC2(CC(n3nc(N4CCC(CN5C[C@@H]6OCCO[C@@H]6C5)CC4(C)C)c(-c4c(Cl)c(Cl)cc5[nH]ncc45)c3C)C2)C1] 1-(6-(4-(5,6-dichloro-1H- indazol-4-yl)-3-(4- (((4aR,7aS)-hexahydro- 6H-[1,4]dioxino[2,3- c]pyrrol-6-yl)methyl)-2,2- dimethylpiperidin-1-yl)-5- methyl-1H-pyrazol-1-yl)- 2-azaspiro[3.3]heptan-2- yl)prop-2-en-1-one | Using Method-12a starting from Intermediate C88b (Step 2), Intermediate D6 (Step 3) and C-SFC- 4 (mobile phase: CO₂/[IPA + 0.1% Et₃N] 75/25); Example 232a = 1st eluting isomer, Example 232b = 2nd eluting isomer. | Example 232b: C-SFC-3 (mobile phase: CO₂/[IPA + 0.025% NH₃] 75/25): Rt = 4.60 min, UPLC-MS-4: Rt = 0.77 min; MS m/z [M + H]⁺ 668.7/670.7/672.7, Example 232a: C-SFC-3 (mobile phase: CO₂/[IPA + 0.025% NH₃] 75/25): Rt = 3.81 min. |
| 233a/ 233b | 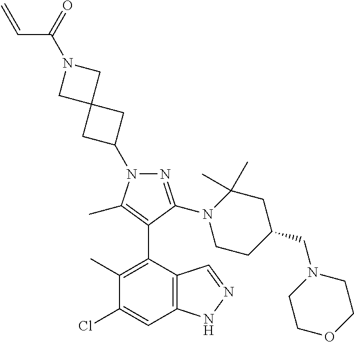 [SMILES: C=CC(=O)N1CC2(CC(n3nc(N4CC[C@@H](CN5CCOCC5)CC4(C)C)c(-c4c(C)c(Cl)cc5[nH]ncc45)c3C)C2)C1] (R)-1-(6-(4-(6-chloro-5- methyl-1H-indazol-4-yl)- 3-(2,2-dimethyl-4- (morpholinomethyl)piperidin- 1-yl)-5-methyl-1H- pyrazol-1-yl)-2- azaspiro[3.3]heptan-2- yl)prop-2-en-1-one | Using Method-12 from Intermediate A70 (Step 1), Intermediate D10 (Step 3) and C-SFC-4 (mobile phase: CO₂/[IPA + 0.1% Et₃N] 75/25); Example 233a = 1st eluting isomer, Example 233b = 2nd eluting isomer. | Example 233b: ¹H NMR (400 MHz, DMSO-d₆) δ 12.9 (s, 1H), 7.57 (s, 1H), 7.55 (s, 1H), 6.30 (m, 1H), 6.09 (m, 1H), 5.67 (m, 1H), 4.72 (m, 1H), 4.34 (s, 1H), 4.28 (s, 1H), 4.05 (s, 1H), 3.99 (s, 1H), 3.51 (m, 4H), 2.90-2.57 (m, 5H), 2.46 (m, 1H), 2.29-2.16 (m, 7H), 1.93 (s, 3H), 1.98-1.89 (m, 2H), 1.74 (m, 1H), 1.45 (m, 1H), 1.32 (m, 1H), 1.29 (s, 3H), 1.09 (s, 1.5H), 1.07 (s, 1.5H), 0.83 (m, 1H), 0.54 (m, 1H). UPLC-MS-4: Rt = 0.78 min; MS m/z [M + H]⁺ 606.4/608.4/610.4; C- SFC-3 (mobile phase: CO₂/[IPA + 0.025% NH₃] 75/25): Rt = 2.92 min, Example 233a: C-SFC-3 (mobile phase: CO₂/[IPA + 0.025% NH₃] 75/25): Rt = 2.21 min. |
| 234a/ 234b | 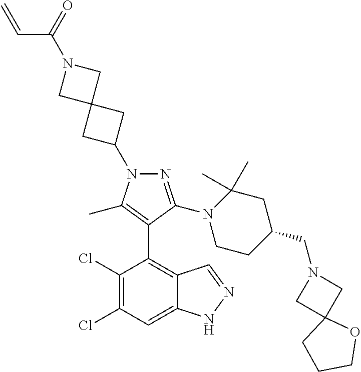 [SMILES: C=CC(=O)N1CC2(CC(n3nc(N4CC[C@@H](CN5CC6(CCCO6)C5)CC4(C)C)c(-c4c(Cl)c(Cl)cc5[nH]ncc45)c3C)C2)C1] (R)-1-(6-(3-(4-((5-oxa-2- azaspiro[3.4]octan-2- yl)methyl)-2,2- dimethylpiperidin-1-yl)-4- (5,6-dichloro-1H-indazol- 4-yl)-5-methyl-1H- pyrazol-1-yl)-2- azaspiro[3.3]heptan-2- yl)prop-2-en-1-one | Using Method-12b starting from Intermediate C89a (Step 2), D6 (Step 3) and C-SFC-20 (mobile phase: CO₂/[IPA + 0.1% Et₃N] 65/35); Example 234a = 1st eluting isomer, Example 234b = 2nd eluting isomer. | Example 234b: UPLC-MS- 4: Rt = 0.82 min; MS m/z [M + H]⁺ 652.3/654.4/ 656.4; C-SFC-3 (mobile phase: CO₂/[IPA + 0.025% NH₃] 70/30): Rt = 2.60 min; Example 234a: C-SFC-3 (mobile phase: CO₂/[IPA + 0.025% NH₃] 70/30): Rt = 1.80 min. |
| 235a/ 235b | 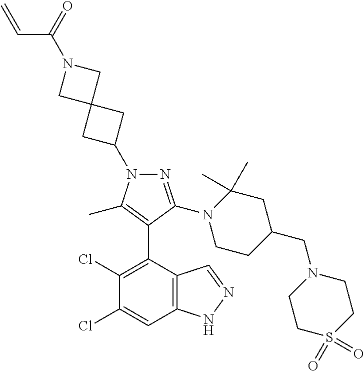 [SMILES: C=CC(=O)N1CC2(CC(n3nc(N4CCC(CN5CCS(=O)(=O)CC5)CC4(C)C)c(-c4c(Cl)c(Cl)cc5[nH]ncc45)c3C)C2)C1] 1-(6-(4-(5,6-dichloro-1H- indazol-4-yl)-3-(4-((1,1- dioxidothiomorpholino) methyl)-2,2- dimethylpiperidin-1-yl)-5- methyl-1H-pyrazol-1-yl)- 2-azaspiro[3.3]heptan-2- yl)prop-2-en-1-one | Using Method-12bc from Intermediate C90b (Step 2), Intermediate D6 (Step 3) and reverse phase chromatography (mobile phase: 20 to 70% CH₃CN in H₂O containing 0.1% TFA); Example 235a = 1st eluting isomer, Example 235b = 2nd eluting isomer. | Example 235b: UPLC-MS- 4: Rt = 0.98 min; MS m/z [M + H]⁺ 674.5/676.5/ 678.5, Example 235a: UPLC-MS-4: Rt = 0.95 min; MS m/z [M + H]⁺ 674.5/ 676.5/678.5. |
| 236a/ 236b | 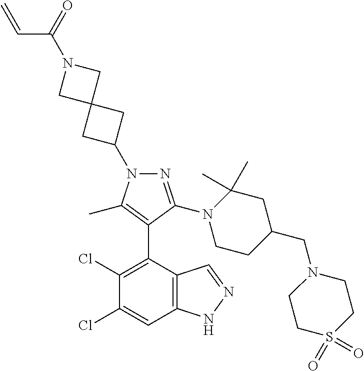 [SMILES: C=CC(=O)N1CC2(CC(n3nc(N4CCC(CN5CCS(=O)(=O)CC5)CC4(C)C)c(-c4c(Cl)c(Cl)cc5[nH]ncc45)c3C)C2)C1] 1-(6-(4-(5,6-dichloro-1H- indazol-4-yl)-3-(4-((1,1- dioxidothiomorpholino) methyl)-2,2- dimethylpiperidin-1-yl)-5- methyl-1H-pyrazol-1-yl)- 2-azaspiro[3.3]heptan-2- yl)prop-2-en-1-one | Using Method-12bc from Intermediate C90a (Step 2), Intermediate D6 (Step 3) and reverse phase chromatography (mobile phase: 20 to 70% CH₃CN in H₂O containing 0.1% TFA); Example 236a = 1st eluting isomer, Example 236b = 2nd eluting isomer. | Example 236a: UPLC-MS- 4: Rt = 0.95 min; MS m/z [M + H]⁺ 674.2/676.2/ 678.2, Example 236b: UPLC-MS-4: Rt = 1.00 min; MS m/z [M + H]⁺ 674.6/ 676.6/678.6. |
| 237a/ 237b | 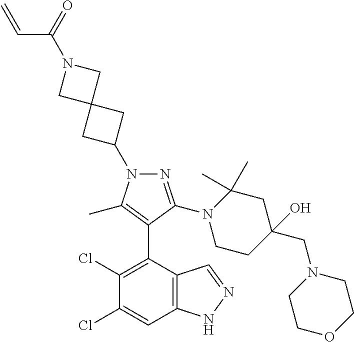 [SMILES: C=CC(=O)N1CC2(CC(n3nc(N4CCC(O)(CN5CCOCC5)CC4(C)C)c(-c4c(Cl)c(Cl)cc5[nH]ncc45)c3C)C2)C1] 1-(6-(4-(5,6-dichloro-1H- indazol-4-yl)-3-(4- hydroxy-2,2-dimethyl-4- (morpholinomethyl)piperidin- 1-yl)-5-methyl-1H- pyrazol-1-yl)-2- azaspiro[3.3]heptan-2- yl)prop-2-en-1-one | Using Method-12a from Intermediate C129b (Step 2), Intermediate D6 (Step 3) and C-SFC- 2 (mobile phase: CO₂/[IPA + 0.1% Et₃N] 65/35); Example 237a = 1st eluting isomer, Example 237b = 2nd eluting isomer. | Example 237b: UPLC-MS- 4: Rt = 0.71 min; MS m/z [M + H]⁺ 642.4/646.4/ 646.4; C-SFC-3 (mobile phase: CO₂/[IPA + 0.025% NH₃] 65/35): Rt = 1.69 min, Example 237a: C-SFC-3 (mobile phase: CO₂/[IPA + 0.025% NH₃] 65/35): Rt = 1.20 min. |
| 238a/ 238b | 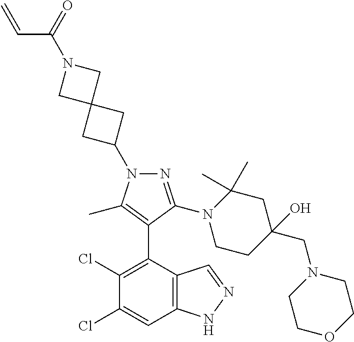 [SMILES: C=CC(=O)N1CC2(CC(n3nc(N4CCC(O)(CN5CCOCC5)CC4(C)C)c(-c4c(Cl)c(Cl)cc5[nH]ncc45)c3C)C2)C1] 1-(6-(4-(5,6-dichloro-1H- indazol-4-yl)-3-(4- hydroxy-2,2-dimethyl-4- (morpholinomethyl)piperidin- 1-yl)-5-methyl-1H- pyrazol-1-yl)-2- azaspiro[3.3]heptan-2- yl)prop-2-en-1-one | Using Method-12a from Intermediate C129a (Step 2), Intermediate D6 (Step 3) and C- HPLC-7 (mobile phase: n-heptane/ CH₂Cl₂/MeOH 65/20/15 + 0.05% Et₂NH); Example 238a = 1st eluting isomer, Example 238b = 2nd eluting isomer. | Example 238a: UPLC-MS- 4: Rt = 0.75 min; MS m/z [M + H]⁺ 642.5/646.5/ 648.5; C-HPLC-6 (mobile phase: n-heptane/ CH₂Cl₂/MeOH 65/20/15 + 0.05% Et₂NH): Rt = 15.2 min, Example 238b: C-HPLC-6 (mobile phase: n-heptane/ CH₂Cl₂/MeOH 65/20/15 + 0.05% Et₂NH) 65/35): Rt = 18.2 min. |
| 239a/ 239b | 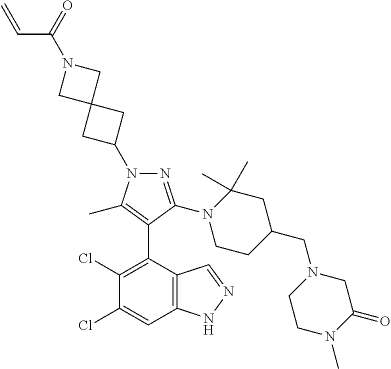 [SMILES: C=CC(=O)N1CC2(CC(n3nc(N4CCC(CN5CCN(C)C(=O)C5)CC4(C)C)c(-c4c(Cl)c(Cl)cc5[nH]ncc45)c3C)C2)C1] 4-((1-(1-(2-acryloyl-2- azaspiro[3.3]heptan-6- yl)-4-(5,6-dichloro-1H- indazol-4-yl)-5-methyl- 1H-pyrazol-3-yl)-2,2- dimethylpiperidin-4- yl)methyl)-1- methylpiperazin-2-one | Using Method Method- 12bc from Intermediate C92b (Step 2) using Intermediate D6 (Step 2) and reverse phase chromatography: (mobile phase: [H₂O + 0.1% TFA]/CH₃CN 90:10 to 0:100 in 21 min); Example 239a = 1st eluting atropisomer Example 239b = 2nd eluting atropisomer | Example 239b: UPLC-MS- 4: Rt = 0.91 min; MS m/z [M + H]⁺ 653.4/655.3/ 657.3; Example 239a: UPLC-MS-4: Rt = 0.87 min; MS m/z [M + H]⁺ 653.4/ 655.3/657.3. |
| 240a/ 240b | 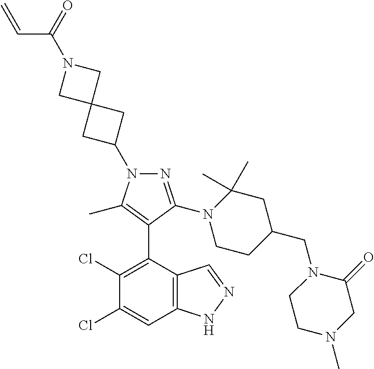 [SMILES: C=CC(=O)N1CC2(CC(n3nc(N4CCC(CN5CCN(C)CC5=O)CC4(C)C)c(-c4c(Cl)c(Cl)cc5[nH]ncc45)c3C)C2)C1] 1-((1-(1-(2-acryloyl-2- azaspiro[3.3]heptan-6- yl)-4-(5,6-dichloro-1H- indazol-4-yl)-5-methyl- 1H-pyrazol-3-yl)-2,2- dimethylpiperidin-4- yl)methyl)-4- methylpiperazin-2-one | Using Method-12bc from Intermediate C93a and Intermediate D6 (Step 2) and RP-HPLC- 4: (mobile phase: [H₂O + 0.1% TFA]/CH₃CN 95:5 to 5:95 in 35 min); Example 240a = 1st eluting atropisomer Example 240b = 2nd eluting atropisomer | Example 240b: UPLC-MS- 4: Rt = 0.85 min; MS m/z [M + H]⁺ 653.3/655.4/ 657.4; RP-HPLC-4: (mobile phase: [H₂O + 0.1% TFA]/CH₃CN 95:5 to 5:95 in 35 min): Rt = 12.5 min, Example 240a: RP-HPLC- 4: (mobile phase: [H₂O + 0.1% TFA]/CH₃CN 95:5 to 5:95 in 35 min): Rt = 11.5 min. |
| 241a/ 241b | 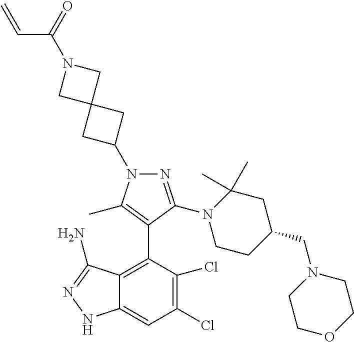 [SMILES: C=CC(=O)N1CC2(CC(n3nc(N4CC[C@@H](CN5CCOCC5)CC4(C)C)c(-c4c(Cl)c(Cl)cc5[nH]nc(N)c45)c3C)C2)C1] (R)-1-(6-(4-(3-amino-5,6- dichloro-1H-indazol-4-yl)- 3-(2,2-dimethyl-4- (morpholinomethyl)piperidin- 1-yl)-5-methyl-1H- pyrazol-1-yl)-2- azaspiro[3.3]heptan-2- yl)prop-2-en-1-one | Using Method-12 and Intermediate D17 (Step 3) and C-SFC-7: (mobile phase: CO₂/[IPA + 0.1% Et₃N] 50/50); Example 241a = 1st eluting atropisomer Example 241b = 2nd eluting atropisomer | Example 241a: UPLC-MS- 4: Rt = 0.76 min; MS m/z [M + H]⁺ 641.2/643.6; C- SFC-8 (mobile phase: CO₂/IPA 50/50): Rt = 1.45 min, Example 241b: C- SFC-8 (mobile phase: CO₂/IPA 50/50): Rt = 2.36 min. |
| 242a/ 242b | 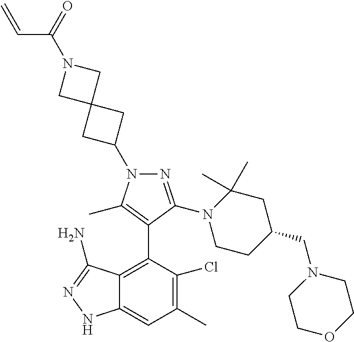 [SMILES: C=CC(=O)N1CC2(CC(n3nc(N4CC[C@@H](CN5CCOCC5)CC4(C)C)c(-c4c(Cl)c(C)cc5[nH]nc(N)c45)c3C)C2)C1] (R)-1-(6-(4-(3-amino-5- chloro-6-methyl-1H- indazol-4-yl)-3-(2,2- dimethyl-4- (morpholinomethyl)piperidin- 1-yl)-5-methyl-1H- pyrazol-1-yl)-2- azaspiro[3.3]heptan-2- yl)prop-2-en-1-one | Using Method-12 from Intermediate D11 (Step 3) and C-SFC-7 (mobile phase: CO₂/[IPA + 0.1% Et₃N] 50/50); Example 242a = 1st eluting isomer, Example 242b = 2nd eluting isomer | Example 242a: ¹H NMR (400 MHz, DMSO-d₆) δ 11.50 (s, 1H), 7.18 (s, 1H), 6.29 (m, 1H), 6.09 (m, 1H), 5.66 (m, 1H), 4.73 (m, 1H), 4.32 (s, 1H), 4.27 (m, 3H), 4.04 (s, 1H), 3.98 (s, 1H), 3.44-3.58 (m, 4H), 2.97 (m, 1H), 2.58-2.81 (m, 5H), 2.40 (s, 3H), 2.16-2.31 (m, 4H), 1.95 (m, 2H), 1.90 (s, 3H), 1.76 (m, 1H), 1.32- 1.45 (m, 2H), 1.23 (m, 3H), 1.04 (m, 3H), 0.88 (m, 1H), 0.62 (m, 1H). UPLC-MS- 2e: Rt = 3.31 min; MS m/z [M + H]⁺ 621.5/623.5; C- SFC-8 (mobile phase: CO₂/[IPA + 0.1% Et₃N] 50/50): Rt = 1.85 min, Example 242b: C-SFC-8 (mobile phase: CO₂/[IPA + 0.1% Et₃N] 50/50): Rt = 3.47 min. |

### Method-13 for the Preparation of Example 243a and 243b: (R)-1-(6-(4-(5,6-dichloro-1H-indazol-4-yl)-3-(2,2-dimethyl-4-((4-(oxetan-3-yl)piperazin-1-yl)methyl)piperidin-1-yl)-5-methyl-1H-pyrazol-1-yl)-2-azaspiro[3.3]heptan-2-yl)prop-2-en-1-one

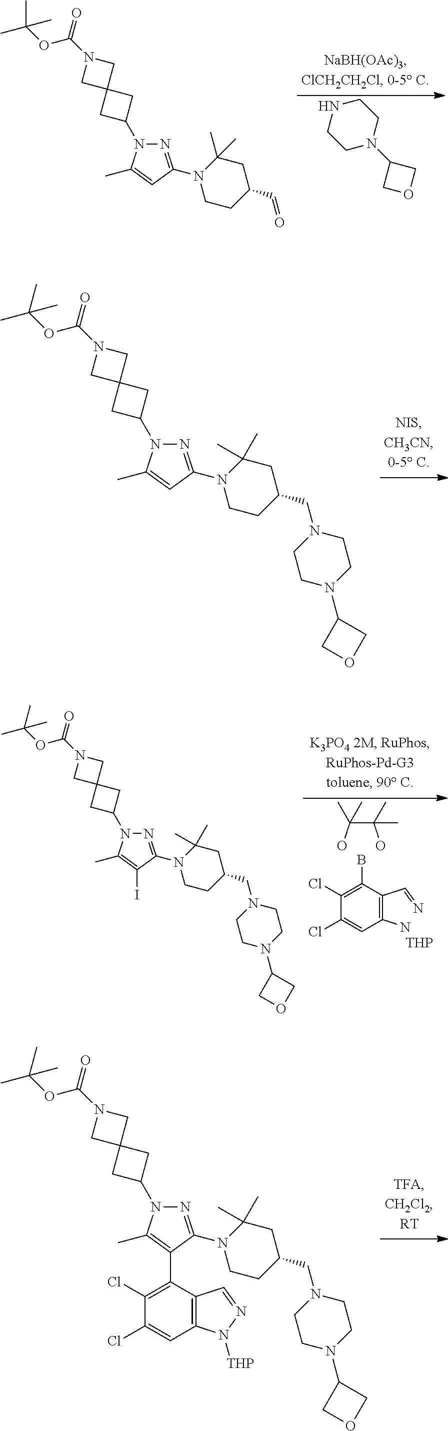

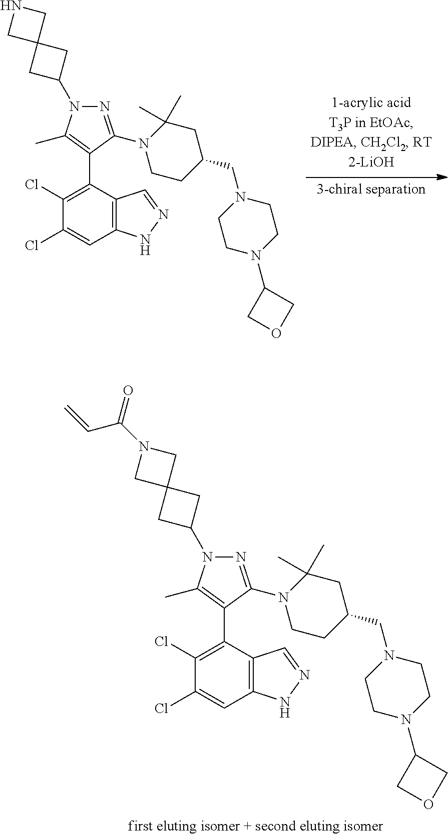 [SMILES: Cc1c(-c2c(Cl)c(Cl)cc3[nH]ncc23)c(N2CC[C@@H](CN3CCN(C4COC4)CC3)CC2(C)C)nn1C1CC2(CNC2)C1 ; C=CC(=O)N1CC2(CC(n3nc(N4CC[C@@H](CN5CCN(C6COC6)CC5)CC4(C)C)c(-c4c(Cl)c(Cl)cc5[nH]ncc45)c3C)C2)C1]

### Step 1: Tert-butyl (R)-6-(3-(2,2-dimethyl-4-((4-(oxetan-3-yl)piperazin-1-yl)methyl)piperidin-1-yl)-5-methyl-1H-pyrazol-1-yl)-2-azaspiro[3.3]heptane-2-carboxylate

A solution of tert-butyl (R)-6-(3-(4-formyl-2,2-dimethylpiperidin-1-yl)-5-methyl-1H-pyrazol-1-yl)-2-azaspiro[3.3]heptane-2-carboxylate (described in the synthesis of Intermediate C116 (Step 1), 6.83 g, 16.4 mmol) and 1-(oxetan-3-yl)piperazine (CAS [1254115-23-5], 2.56 g, 18.0 mmol) in dichloroethane (80 mL) was stirred under a nitrogen atmosphere at 0-5° C. for 10 min. Sodium triacetoxyborohydride (5.20 g, 24.6 mmol) was added and the reaction mixture was stirred at 0-5° C. for 30 min. The RM was poured into a sat. aq. NaHCO₃ solution and extracted with CH₂Cl₂ (×3). The combined organic layers were washed with a sat. aq. NaHCO₃ solution, dried (phase separator) and concentrated. The crude residue was purified by normal phase chromatography (eluent: MeOH in CH₂Cl₂ 0 to 14%) to give the title compound. UPLC-MS-4: Rt=0.64 min; MS m/z [M+H]⁺ 543.6.

### Step 2: Tert-butyl (R)-6-(3-(2,2-dimethyl-4-((4-(oxetan-3-yl)piperazin-1-yl)methyl)piperidin-1-yl)-4-iodo-5-methyl-1H-pyrazol-1-yl)-2-azaspiro[3.3]heptane-2-carboxylate

To an ice-cooled solution of tert-butyl (R)-6-(3-(2,2-dimethyl-4-((4-(oxetan-3-yl)piperazin-1-yl)methyl)piperidin-1-yl)-5-methyl-1H-pyrazol-1-yl)-2-azaspiro[3.3]heptane-2-carboxylate (Step 1, 6.81 g, 12.6 mmol) in CH₃CN (120 mL) was added NIS (2.97 g, 13.2 mmol) and the mixture was stirred under N₂ atmosphere at 0° C. After completion (10 min), the reaction mixture was poured into 10% Na₂S₂O₃ aq. solution and extracted with CH₂Cl₂ (×2). The combined organic layers were washed with an aq. sat. NaHCO₃ solution, dried (phase separator) and concentrated to give the title product which was used without further purification in the next step. UPLC-MS-4: Rt=0.96 min; MS m/z [M+H]⁺ 669.5.

### Step 3: Tert-butyl 6-(4-(5,6-dichloro-1-(tetrahydro-2H-pyran-2-yl)-1H-indazol-4-yl)-3-((R)-2,2-dimethyl-4-((4-(oxetan-3-yl)piperazin-1-yl)methyl)piperidin-1-yl)-5-methyl-1H-pyrazol-1-yl)-2-azaspiro[3.3]heptane-2-carboxylate

To a mixture of tert-butyl (R)-6-(3-(2,2-dimethyl-4-((4-(oxetan-3-yl)piperazin-1-yl)methyl)piperidin-1-yl)-4-iodo-5-methyl-1H-pyrazol-1-yl)-2-azaspiro[3.3]heptane-2-carboxylate (Step 2, 8.41 g, 12.6 mmol), 5,6-dichloro-1-(tetrahydro-2H-pyran-2-yl)-4-(4,4,5,5-tetramethyl-1,3,2-dioxaborolan-2-yl)-1H-indazole (Intermediate D6, 6.49 g, 16.35 mmol), RuPhos (599 mg, 1.26 mmol) and RuPhos-Pd-G3 (1.07 g, 1.26 mmol) in toluene (120 mL) was added K₃PO₄ (2 M in water, 18.9 mL, 37.8 mmol) and the reaction mixture was stirred at 85° C. for 1.5 h under nitrogen atmosphere. The reaction mixture was poured into a sat. aq. NaHCO₃ solution and extracted with EtOAc (×3). The combined organic extracts were dried (phase separator) and concentrated. The crude residue was diluted with THF (100 mL), SiliaMetS®Thiol (5.03 mmol) was added and the mixture swirled for 1 h at 40° C. The mixture was filtered, the filtrate was concentrated and the crude residue was purified by normal phase chromatography (eluent: EtOAc in n-heptane 0 to 100% then 10% MeOH in CH₂Cl₂) to give the title compound as a brown foam. UPLC-MS-4: Rt=1.18 and 1.20 min; MS m/z [M+H]⁺ 811.4/813.4/815.4.

### Step 4: (R)-5,6-dichloro-4-(3-(2,2-dimethyl-4-((4-(oxetan-3-yl)piperazin-1-yl)methyl)piperidin-1-yl)-5-methyl-1-(2-azaspiro[3.3]heptan-6-yl)-1H-pyrazol-4-yl)-1H-indazole

To a solution of tert-butyl 6-(4-(5,6-dichloro-1-(tetrahydro-2H-pyran-2-yl)-1H-indazol-4-yl)-3-((R)-2,2-dimethyl-4-((4-(oxetan-3-yl)piperazin-1-yl)methyl)piperidin-1-yl)-5-methyl-1H-pyrazol-1-yl)-2-azaspiro[3.3]heptane-2-carboxylate (Step 3, 7.59 g, 9.37 mmol) in CH₂Cl₂ (31 mL) was added TFA (22.6 mL, 281 mmol) and the solution was stirred at RT for 1.5 h. The RM was concentrated, co-evaporated with CH₂Cl₂ (×2) and dried under high vacuum to give the title compound as a trifluoroacetate salt which was used in the next step without further purification. UPLC-MS-4: Rt=0.47 and 0.53 min; MS m/z [M+H]⁺ 627.4/629.4/631.4.

### Step 5: (R)-1-(6-(4-(5,6-dichloro-1H-indazol-4-yl)-3-(2,2-dimethyl-4-((4-(oxetan-3-yl)piperazin-1-yl)methyl)piperidin-1-yl)-5-methyl-1H-pyrazol-1-yl)-2-azaspiro[3.3]heptan-2-yl)prop-2-en-1-one

A mixture of acrylic acid (0.77 mL, 11.2 mmol), propylphosphonic anhydride (50% in EtOAc, 8.27 mL, 14.0 mmol) and DIPEA (32.7 mL, 187 mmol) in CH₂Cl₂ (125 mL) was stirred for 15 min under argon atmosphere and added to an ice-cooled solution of (R)-5,6-dichloro-4-(3-(2,2-dimethyl-4-((4-(oxetan-3-yl)piperazin-1-yl)methyl)piperidin-1-yl)-5-methyl-1-(2-azaspiro[3.3]heptan-6-yl)-1H-pyrazol-4-yl)-1H-indazole (Step 4, 9.36 mmol) in CH₂Cl₂ (62.5 mL). The reaction mixture was stirred at RT under argon for 10 min and poured into a sat. aq. NaHCO₃ solution. The layers were separated and the aqueous layer was back-extracted with CH₂Cl₂ (×3). The combined organic layers were washed with a sat. aq. NaHCO₃ solution, dried (phase separator) and concentrated under reduced pressure. The crude residue was dissolved in THF (90 mL) and LiOH (2 M, 23.4 mL, 46.8 mmol) was added. The mixture was stirred at RT for 1 h, then was poured into a sat. aq. NaHCO₃ solution and extracted with EtOAc. The aqueous layer was then extracted with CH₂Cl₂ (×2) and the combined organic layers were dried (phase separator) and concentrated. The residue was purified by normal phase chromatography (eluent: MeOH in CH₂Cl₂ from 0 to 15%) to give fraction A containing the first eluting isomer and fraction B containing the second eluting isomer. Fraction B was purified again by reverse phase chromatography (eluent A: H₂O+0.1% TFA, B: CH₃CN, gradient: 10% to 100%, flow: 150 mL/min) to give after extraction with a sat. aq. solution of NaHCO₃ and CH₂Cl₂, evaporation of volatils and lyophilization from a mixture of CH₃CN and water, Example 243b as the second eluting isomer (white solid): ¹H NMR (400 MHz, DMSO-d₆) δ 13.2 (s, 1H), 7.79 (s, 1H), 7.63 (s, 1H), 6.31 (m, 1H), 6.10 (m, 1H), 5.67 (m, 1H), 4.76 (m, 1H), 4.50 (t, 2H), 4.39 (t, 2H), 4.34 (s, 1H), 4.29 (s, 1H), 4.05 (s, 1H), 4.00 (s, 1H), 3.34 (m, 1H), 3.17 (m, 1H), 2.79 (m, 1H), 2.78-2.63 (m, 4H), 2.36-2.12 (m, 8H), 2.01 (s, 3H), 1.96-1.89 (m, 2H), 1.76 (m, 1H), 1.53 (m, 1H), 1.34 (m, 1H), 1.12 (s, 3H), 0.72 (s, 1.5H), 0.70 (s, 1.5H), 0.72-0.66 (m, 1H), 0.60 (m, 1H). UPLC-MS-2e: Rt=3.86 min; MS m/z [M+H]⁺ 681.3/683.3/685.3. Fraction A was purified again by normal phase chromatography (eluent: MeOH in CH₂Cl₂ from 0 to 16%) to give after lyophilization (CH₃CN/water), Example 243a as the first eluting isomer (white solid): UPLC-MS-2e: Rt=3.54 min; MS m/z [M+H]⁺ 681.3/683.3/685.3.

Method-13a: similar to Method-13 except that Step 1 was performed in MeOH instead of ClCH₂CH₂Cl.

Method-13b: similar to Method-13 except that Step 3 was performed using chloro(crotyl)(tri-tert-butylphosphine)Pd(II) (CAS [1334497-00-5]) as a catalyst in dioxane as solvent.

Method-13c: similar to Method-13 except that Step 3 was performed using 0.2 eq. of cataCXium-A-Pd-G3 (CAS [1651823-59-4]) as catalyst, cyclopentylmethyl ether (138 mg, 0,190 mmol) as solvent and 3 eq. of aq. KOH (1 M) as base at 60° C.

Method-13d: similar to Method-13 except that Step 5 was performed using acryloyl chloride and NaHCO₃ as described in Method-8 Step 3.

Method-13e: similar to Method-13 except that Step 2 was performed in THF instead of CH₃CN.

Method-13f: similar to Method-13 except that Step 3 was performed in dioxane instead of toluene.

The following examples 244a to 320b were prepared using analogous methods to Method-13 from intermediates (in Step 1, 2 or 3) described in the intermediates synthesis section or commercially available. When the final separation of isomers is performed by preparative HPLC using acidic conditions the purified fractions were extracted with a sat. aq. NaHCO₃ solution before lyophilization from a mixture of CH₃CN/H₂O to give the title compound as a free base.

| | | Method, intermediates | |
|---|---|---|---|
| | | (Step 1, 3 or 3) and chiral | |
| | | separation conditions | |
| Example | Structure | used and order of elution | Characterizing data |
| 244a/ 244b | 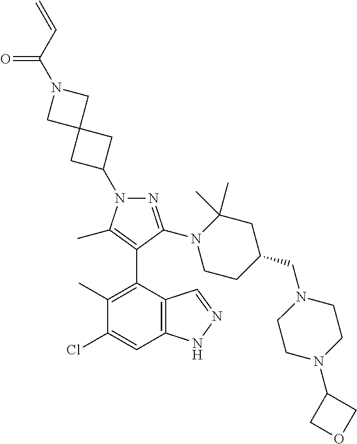 [SMILES: C=CC(=O)N1CC2(CC(n3nc(N4CC[C@@H](CN5CCN(C6COC6)CC5)CC4(C)C)c(-c4c(C)c(Cl)cc5[nH]ncc45)c3C)C2)C1] (R)-1-(6-(4-(6-chloro-5- methyl-1H-indazol-4-yl)-3- (2,2-dimethyl-4-((4-(oxetan- 3-yl)piperazin-1- yl)methyl)piperidin-1-yl)-5- methyl-1H-pyrazol-1-yl)-2- azaspiro[3.3]heptan-2- yl)prop-2-en-1-one | Using Method-13c and CAS [1254115-23-5] (Step 1), Intermediate D10 (Step 3) and RP-HPLC-2 (mobile phase: 5 to 50% CH₃CN in H₂O containing 0.1% TFA); Example 244a = 1st eluting isomer, Example 244b = 2nd eluting isomer. | Example 244b: ¹H NMR (400 MHz, DMSO-d₆) δ 13.0 (s, 1H), 7.58 (s, 1H), 7.56 (s, 1H), 6.30 (m, 1H), 6.10 (m, 1H), 5.67 (m, 1H), 4.72 (m, 1H), 4.49 (m, 2H), 4.37 (m, 2H), 4.34 (s, 1H), 4.29 (m, 1H), 4.06 (s, 1H), 3.99 (m, 1H), 2.89- 2.66 (m, 5H), 2.65- 2.57 (m, 1H), 2.51- 2.42 (m, 1H), 2.36- 2.11 (m, 7H), 2.24 (s, 3H), 1.98-1.91 (m, 2H), 1.94 (s, 3H), 1.74 (m, 1H), 1.65 (m, 1H), 1.36-1.24 (m, 2H), 1.30 (s, 3H), 1.09 (s, 1.5H), 1.07 (s, 1.5H), 0.81 (m, 1H), 0.53 (m, 1H). UPLC-MS-2e: Rt = 3.76 min; MS m/z [M + H]⁺ 661.3/663.3, Example 244a: UPLC-MS-2e: Rt = 3.48 min; MS m/z [M + H]⁺ 661.3/663.3. |
| 245a/ 245b | 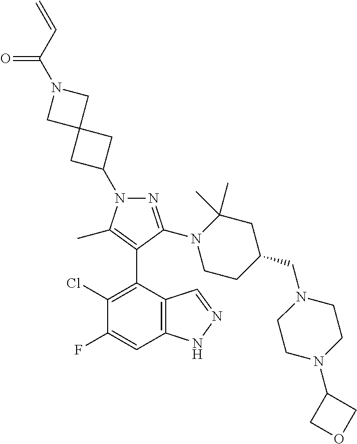 [SMILES: C=CC(=O)N1CC2(CC(n3nc(N4CC[C@@H](CN5CCN(C6COC6)CC5)CC4(C)C)c(-c4c(Cl)c(F)cc5[nH]ncc45)c3C)C2)C1] (R)-1-(6-(4-(5-chloro-6- fluoro-1H-indazol-4-yl)-3- (2,2-dimethyl-4-((4-(oxetan- 3-yl)piperazin-1- yl)methyl)piperidin-1-yl)-5- methyl-1H-pyrazol-1-yl)-2- azaspiro[3.3]heptan-2- yl)prop-2-en-1-one | Using Method-13 and CAS [1254115-23-5] (Step 1), Intermediate D9 (Step 3) and RP-HPLC-2 (mobile phase: 7 to 37% CH₃CN in H₂O containing 0.1% TFA); Example 245a = 1st eluting isomer, Example 245b = 2nd eluting isomer. | Example 245b: ¹H NMR (400 MHz, DMSO-d₆) δ 13.2 (s, 1H), 7.64 (s, 1H), 7.50 (d, 1H), 6.32 (m, 1H), 6.10 (m, 1H), 5.68 (m, 1H), 4.76 (m, 1H), 4.49 (m, 2H), 4.39 (m, 2H), 4.34 (s, 1H), 4.29 (s, 1H), 4.06 (s, 1H), 4.00 (s, 1H), 3.39- 3.31 (m, 1H), 3.22- 3.12 (m, 1H), 2.83- 2.65 (m, 5H), 2.36- 2.11 (m, 8H), 2.03 (s, 3H), 1.94 (m, 2H), 1.77 (m, 1H), 1.54 (m, 1H), 1.35 (m, 1H), 1.12 (s, 3H), 0.77- 0.66 (m, 1H), 0.72 (s, 1.5H), 0.70 (s, 1.5H), 0.60 (m, 1H). UPLC- MS-2e: Rt = 3.55 min; MS m/z [M + H]⁺ 665.4/ 667.4, Example 245a: UPLC-MS-2e: Rt = 3.23 min; MS m/z [M + H]⁺ 665.4/667.4. |
| 246a/ 246b | 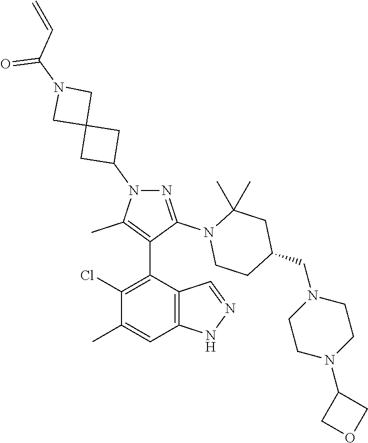 [SMILES: C=CC(=O)N1CC2(CC(n3nc(N4CC[C@@H](CN5CCN(C6COC6)CC5)CC4(C)C)c(-c4c(Cl)c(C)cc5[nH]ncc45)c3C)C2)C1] (R)-1-(6-(4-(5-chloro-6- methyl-1H-indazol-4-yl)-3- (2,2-dimethyl-4-((4-(oxetan- 3-yl)piperazin-1- yl)methyl)piperidin-1-yl)-5- methyl-1H-pyrazol-1-yl)-2- azaspiro[3.3]heptan-2- yl)prop-2-en-1-one | Using Method-13 and CAS [1254115-23-5] (Step 1), Intermediate D1 (Step 3) and RP-HPLC-2 (mobile phase: 5 to 40% CH₃CN in H₂O containing 0.1% TFA); Example 246a = 1st eluting isomer, Example 246b = 2nd eluting isomer. | Example 246b: ¹H NMR (400 MHz, DMSO-d₆) δ 12.9 (s, 1H), 7.53 (s, 1H), 7.43 (s, 1H), 6.30 (m, 1H), 6.09 (m, 1H), 5.66 (m, 1H), 4.73 (m, 1H), 4.49 (m, 2H), 4.38 (m, 2H), 4.34 (s, 1H), 4.28 (s, 1H), 4.05 (s, 1H), 3.99 (s, 1H), 3.39- 3.31 (m, 1H), 3.22- 3.12 (m, 1H), 2.83- 2.62 (m, 5H), 2.46 (s, 3H), 2.37-2.10 (m, 8H), 1.98 (s, 3H), 1.95-1.86 (m, 2H), 1.76 (m, 1H), 1.50 (m, 1H), 1.31 (m, 1H), 1.11 (s, 3H), 0.77- 0.55 (m, 2H), 0.73 (s, 1.5H), 0.72 (s, 1.5H). UPLC-MS-2e: Rt = 3.59 min; MS m/z [M + H]⁺ 661.4/663.4, Example 246a: UPLC-MS-2e: Rt = 3.35 min; MS m/z [M + H]⁺ 661.4/663.4. |
| 247a/ 247b | 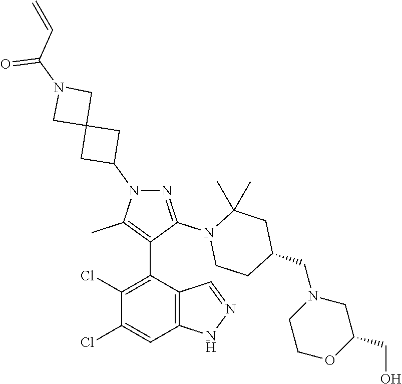 [SMILES: C=CC(=O)N1CC2(CC(n3nc(N4CC[C@@H](CN5CCO[C@@H](CO)C5)CC4(C)C)c(-c4c(Cl)c(Cl)cc5[nH]ncc45)c3C)C2)C1] 1-(6-(4-(5,6-dichloro-1H- indazol-4-yl)-3-((R)-4-(((R)-2- (hydroxymethyl)morpholino) methyl)-2,2- dimethylpiperidin-1-yl)-5- methyl-1H-pyrazol-1-yl)-2- azaspiro[3.3]heptan-2- yl)prop-2-en-1-one | Using Method-13 and CAS [156925-22-3] (Step 1), Intermediate D6 (Step 3) and RP-HPLC-2 (mobile phase: 10 to 45% CH₃CN in H₂O containing 0.1% TFA); Example 247a = 1st eluting isomer, Example 247b = 2nd eluting isomer. | Example 247b: UPLC-MS-2e: Rt = 3.82 min; MS m/z [M + H]⁺ 656.3/658.3/ 660.3, Example 247a: UPLC-MS-2e: Rt = 3.55 min; MS m/z [M + H]⁺ 656.3/658.3/ 660.3. |
| 248a/ 248b | 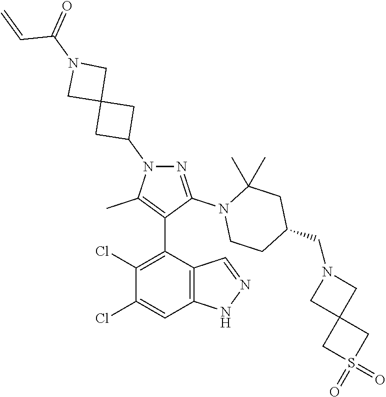 [SMILES: C=CC(=O)N1CC2(CC(n3nc(N4CC[C@@H](CN5CC6(C5)CS(=O)(=O)C6)CC4(C)C)c(-c4c(Cl)c(Cl)cc5[nH]ncc45)c3C)C2)C1] (R)-1-(6-(4-(5,6-dichloro-1H- indazol-4-yl)-3-(4-((2,2- dioxido-2-thia-6- azaspiro[3.3]heptan-6- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and CAS [1427388-39-3] (Step 1), Intermediate D6 (Step 3) and RP-HPLC-2 (mobile phase: 10 to 40% CH₃CN in H₂O containing 0.1% TFA); Example 248a = 1st eluting isomer, Example 248b = 2nd eluting isomer. | Example 248b: UPLC-MS-2e: Rt = 3.80 min; MS m/z [M + H]⁺ 686.2/688.2/ 690.2, Example 248a: UPLC-MS-2e: Rt = 3.42 min; MS m/z [M + H]⁺ 686.2/688.2/ 690.2. |
| 249a/ 249b | 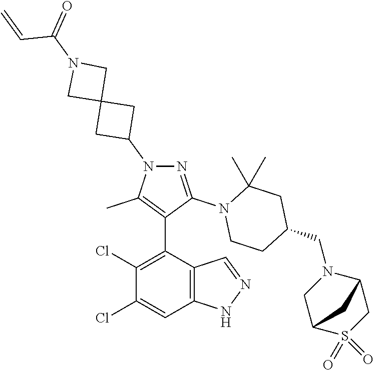 [SMILES: C=CC(=O)N1CC2(CC(n3nc(N4CC[C@@H](CN5C[C@@H]6C[C@H]5CS6(=O)=O)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((1S,4S)-2,2-dioxido-2-thia- 5-azabicyclo[2.2.1]heptan-5- yl)methyl)-2,2- dimethylpiperidin-1-yl)-5- methyl-1H-pyrazol-1-yl)-2- azaspiro [3.3]heptan-2- yl)prop-2-en-1-one | Using Method-13 and CAS [1481613-21-1] (Step 1), Intermediate D6 (Step 3) and RP-HPLC-2 (mobile phase: 10 to 40% CH₃CN in H₂O containing 0.1% TFA); Example 249a = 1st eluting isomer, Example 249b = 2nd eluting isomer. | Example 249b: UPLC-MS-2e: Rt = 4.46 min; MS m/z [M + H]⁺ 686.2/688.2/ 690.2, Example 249a: UPLC-MS-2e: Rt = 4.27 min; MS m/z [M + H]⁺ 686.2/688.2/ 690.2. |
| 250a/ 250b | 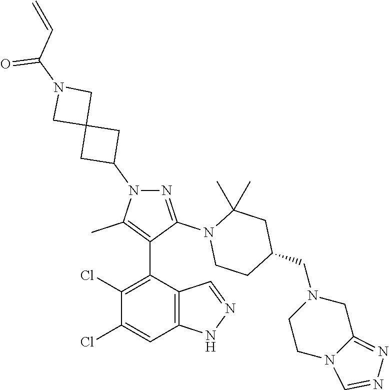 [SMILES: C=CC(=O)N1CC2(CC(n3nc(N4CC[C@@H](CN5CCn6cnnc6C5)CC4(C)C)c(-c4c(Cl)c(Cl)cc5[nH]ncc45)c3C)C2)C1] (R)-1-(6-(4-(5,6-dichloro-1H- indazol-4-yl)-3-(4-((5,6- dihydro-[1,2,4]triazolo[4,3- alpyrazin-7(8H)-yl)methyl)- 2,2-dimethylpiperidin-1-yl)-5- methyl-1H-pyrazol-1-yl)-2- azaspiro[3.3]heptan-2- yl)prop-2-en-1-one | Using Method-13b and CAS [345311-09-3] (Step 1), Intermediate D6 (Step 3) and C-SFC-4 (mobile phase: (CO₂/[IPA + 0.1% Et₃N]: 60/40); Example 250a = 1st eluting isomer, , Example 250b = 2nd eluting isomer. | , Example 250b: UPLC-MS-2e: Rt = 4.55 min; MS m/z [M + H]⁺ 663.3/665.3/ 667.3, Example 250a: UPLC-MS-2e: Rt = 4.26 min; MS m/z [M + H]⁺ 663.3/665.3/ 667.3. |
| 251a/ 251b | 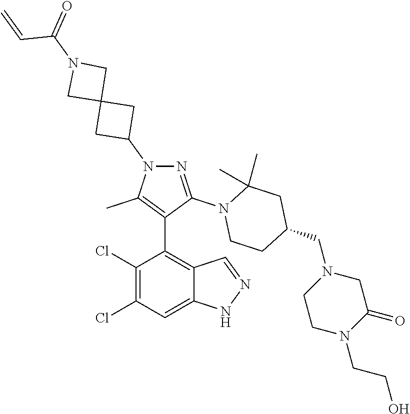 [SMILES: C=CC(=O)N1CC2(CC(n3nc(N4CC[C@@H](CN5CCN(CCO)C(=O)C5)CC4(C)C)c(-c4c(Cl)c(Cl)cc5[nH]ncc45)c3C)C2)C1] (R)-4-((1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-1-(2- hydroxyethyl)piperazin-2-one | Using Method-13 and CAS [59702-23-7] (Step 1), Intermediate D6 (Step 3) and RP-HPLC-2 (mobile phase: 8 to 38% CH₃CN in H₂O containing 0.1% TFA); Example 251a = 1st eluting isomer, Example 251b = 2nd eluting isomer. | Example 251b: UPLC-MS-2e: Rt = 4.19 min; MS m/z [M + H]⁺ 683.3/685.3/ 687.3, Example 251a: UPLC-MS-2e: Rt = 3.90 min; MS m/z [M + H]⁺ 683/685.3/ 687.3. |
| 252a/ 252b | 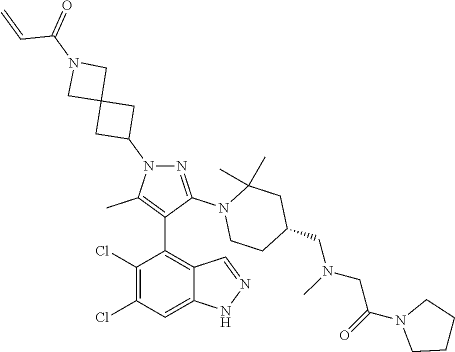 [SMILES: C=CC(=O)N1CC2(CC(n3nc(N4CC[C@@H](CN(C)CC(=O)N5CCCC5)CC4(C)C)c(-c4c(Cl)c(Cl)cc5[nH]ncc45)c3C)C2)C1] (R)-1-(6-(4-(5,6-dichloro-1H- indazol-4-yl)-3-(2,2-dimethyl- 4-((methyl(2-oxo-2- (pyrrolidin-1- yl)ethyl)amino)methyl)piperidin- 1-yl)-5-methyl-1H-pyrazol- 1-yl)-2-azaspiro[3.3]heptan- 2-yl)prop-2-en-1-one | Using Method-13 and CAS [144685-61-0] (Step 1), Intermediate D6 (Step 3) and RP-HPLC-2 (mobile phase: 10 to 45% CH₃CN in H₂O containing 0.1% TFA); Example 252a = 1st eluting isomer, Example 252b = 2nd eluting isomer. | Example 252b: UPLC-MS-2e: Rt = 3.99 min; MS m/z [M + H]⁺ 679.4/681.4/ 683.4, Example 252a: UPLC-MS-2e: Rt = 3.71 min; MS m/z [M + H]⁺ 679.4/681.4/ 683.4. |
| 253a/ 253b | 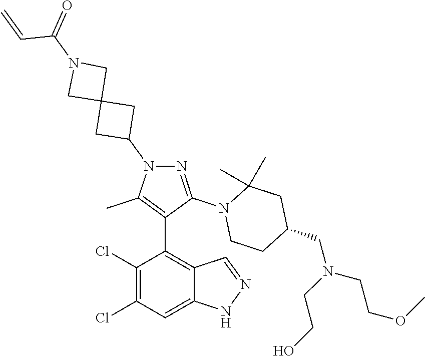 [SMILES: C=CC(=O)N1CC2(CC(n3nc(N4CC[C@@H](CN(CCO)CCOC)CC4(C)C)c(-c4c(Cl)c(Cl)cc5[nH]ncc45)c3C)C2)C1] (R)-1-(6-(4-(5,6-dichloro-1H- indazol-4-yl)-3-(4-(((2- hydroxyethyl)(2- methoxyethyl)amino)methyl)- 2,2-dimethylpiperidin-1-yl)-5- methyl-1H-pyrazol-1-yl)-2- azaspiro[3.3]heptan-2- yl)prop-2-en-1-one | Using Method-13 and CAS [3485-05-0] (Step 1), Intermediate D6 (Step 3) and RP-HPLC-2 (mobile phase: 10 to 40% CH₃CN in H₂O containing 0.1% TFA); Example 253a = 1st eluting isomer, Example 253b = 2nd eluting isomer. | Example 253b: UPLC-MS-2e: Rt = 3.96 min; MS m/z [M + H]⁺ 658.3/660.3/ 662.3, Example 253a: UPLC-MS-2e: Rt = 3.67 min; MS m/z [M + H]⁺ 658.3/660.3/ 662.3,. |
| 254a/ 254b | 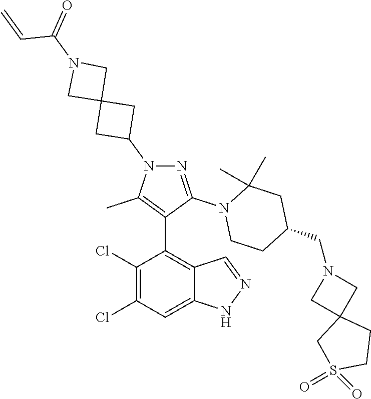 [SMILES: C=CC(=O)N1CC2(CC(n3nc(N4CC[C@@H](CN5CC6(CCS(=O)(=O)C6)C5)CC4(C)C)c(-c4c(Cl)c(Cl)cc5[nH]ncc45)c3C)C2)C1] (R)-1-(6-(4-(5,6-dichloro-1H- indazol-4-yl)-3-(4-((6,6- dioxido-6-thia-2- azaspiro[3.4]octan-2- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and 6- thia-2-azaspiro[3.4] octane 6,6-dioxide hydrochloride salt (Step 1), Intermediate D6 (Step 3) and RP-HPLC- 2 (mobile phase: 10 to 40% CH₃CN in H₂O containing 0.1% TFA); Example 254a = 1st eluting isomer, Example 254b = 2nd eluting isomer. | Example 254b: UPLC-MS-2e: Rt = 3.83 min; MS m/z [M + H]⁺ 700.3/702.3/ 704.3, Example 254a: UPLC-MS-2e: Rt = 3.49 min; MS m/z [M + H]⁺ 700.3/702.3/ 704.3. |
| 255a/ 255b | 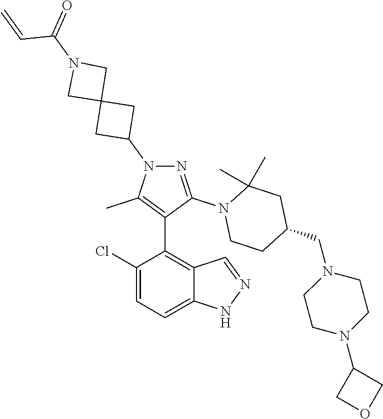 [SMILES: C=CC(=O)N1CC2(CC(n3nc(N4CC[C@@H](CN5CCN(C6COC6)CC5)CC4(C)C)c(-c4c(Cl)ccc5[nH]ncc45)c3C)C2)C1] (R)-1-(6-(4-(5-chloro-1H- indazol-4-yl)-3-(2,2-dimethyl- 4-((4-(oxetan-3-yl)piperazin- 1-yl)methyl)piperidin-1-yl)-5- methyl-1H-pyrazol-1-yl)-2- azaspiro[3.3]heptan-2- yl)prop-2-en-1-one | Using Method-13 and CAS [1254115-23-5] (Step 1), Intermediate D7 (Step 3) and RP-HPLC-2 (mobile phase: 5 to 35% CH₃CN in H₂O containing 0.1% TFA); Example 255a = 1st eluting isomer, Example 255b = 2nd eluting isomer. | Example 255b: UPLC-MS-2e: Rt = 3.38 min; MS m/z [M + H]⁺ 647.4/649.4, Example 255a: UPLC-MS-2e: Rt = 3.08 min; MS m/z [M + H]⁺ 647.4/649.4. |
| 256a/ 256b | 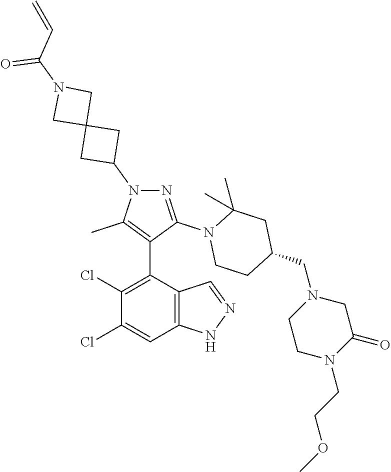 [SMILES: C=CC(=O)N1CC2(CC(n3nc(N4CC[C@@H](CN5CCN(CCOC)C(=O)C5)CC4(C)C)c(-c4c(Cl)c(Cl)cc5[nH]ncc45)c3C)C2)C1] (R)-4-((1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-1-(2- methoxyethyl)piperazin-2- one | Using Method-13e and 1- (2-methoxyethyl)piperazin- 2-one hydrochloride (Step 1), Intermediate D6 (Step 3) and normal phase chromatography (eluent: MeOH in CH₂Cl₂ from 0 to 10%); Example 256a = 1st eluting isomer, Example 256b = 2nd eluting isomer. | Example 256b: UPLC-MS-4: Rt = 0.92 min; MS m/z [M + H]⁺ 697.4/699.4/ 701.4, Example 256a: UPLC-MS-4: Rt = 0.90 min; MS m/z [M + H]⁺ 697.4/699.4/ 701.4. |
| 257a/ 257b | 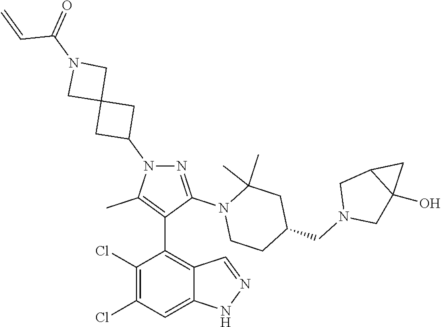 [SMILES: C=CC(=O)N1CC2(CC(n3nc(N4CC[C@@H](CN5CC6CC6(O)C5)CC4(C)C)c(-c4c(Cl)c(Cl)cc5[nH]ncc45)c3C)C2)C1] 1-(6-(4-(5,6-dichloro-1H- indazol-4-yl)-3-((4R)-4-((1- hydroxy-3- azabicyclo[3.1.0]hexan-3- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and Intermediate C120a (Step 2), Intermediate D6 (Step 3) and RP-HPLC-2 (mobile phase: 10 to 40% CH₃CN in H₂O containing 0.1% TFA); Example 257a = 1st eluting isomer, Example 257b = 2nd eluting isomer. | Example 257b: UPLC-MS-2e: Rt = 3.83 min; MS m/z [M + H]⁺ 638.3/640.3/ 642.3, Example 257a: UPLC-MS-2e: Rt = 3.54 min; MS m/z [M + H]⁺ 638.3/640.3/ 642.3. |
| 258a/ 258b | 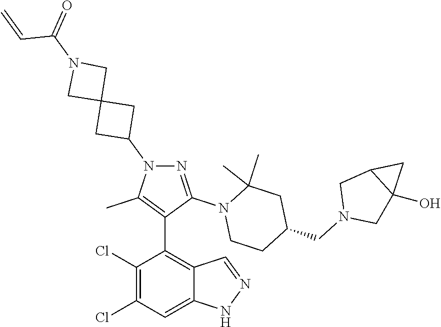 [SMILES: C=CC(=O)N1CC2(CC(n3nc(N4CC[C@@H](CN5CC6CC6(O)C5)CC4(C)C)c(-c4c(Cl)c(Cl)cc5[nH]ncc45)c3C)C2)C1] 1-(6-(4-(5,6-dichloro-1H- indazol-4-yl)-3-((4R)-4-((1- hydroxy-3- azabicyclo[3.1.0]hexan-3- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and Intermediate C120b (Step 2), Intermediate D6 (Step 3) and C-SFC-4 (mobile phase: (CO₂/[IPA + 0.05% Et₃N]: 70/30); Example 258a = 1st eluting isomer, Example 258b = 2nd eluting isomer. | Example 258b: UPLC-MS-2e: Rt = 3.83 min; MS m/z [M + H]⁺ 638.3/640.3/ 642.3, Example 258a: UPLC-MS-2e: Rt = 3.54 min; MS m/z [M + H]⁺ 638.3/640.3/ 642.3. |
| 259a/ 259b | 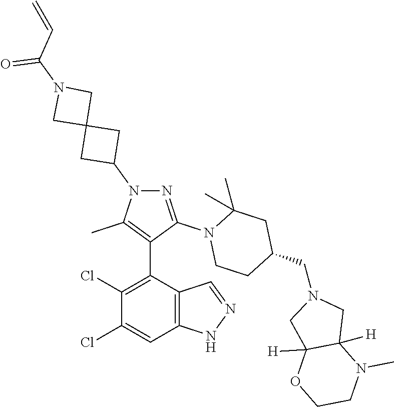 [SMILES: C=CC(=O)N1CC2(CC(n3nc(N4CC[C@@H](CN5CC6OCCN(C)C6C5)CC4(C)C)c(-c4c(Cl)c(Cl)cc5[nH]ncc45)c3C)C2)C1] 1-(6-(4-(5,6-dichloro-1H- indazol-4-yl)-3-((R)-2,2- dimethyl-4-(((4aR*,7aS*)-4- methylhexahydropyrrolo[3,4- b][1,4]oxazin-6(2H)- yl)methyl)piperidin-1-yl)-5- methyl-1H-pyrazol-1-yl)-2- azaspiro[3.3]heptan-2- yl)prop-2-en-1-one | Using Method-13e and Intermediate C118a (Step 2), using Intermediate D6 (Step 3) and reverse phase chromatography (mobile phase: 5 to 50% CH₃CN in H₂O containing 0.1% TFA); Example 259a = 1st eluting isomer, Example 259b = 2nd eluting isomer. | Example 259b: UPLC-MS-4: Rt = 0.78 min; MS m/z [M + H]⁺ 681.4/683.4/ 685.4, Example 259a: UPLC-MS-4: Rt = 0.73 min; MS m/z [M + H]⁺ 681.4/683.4/ 685.4. |
| 260a/ 260b | 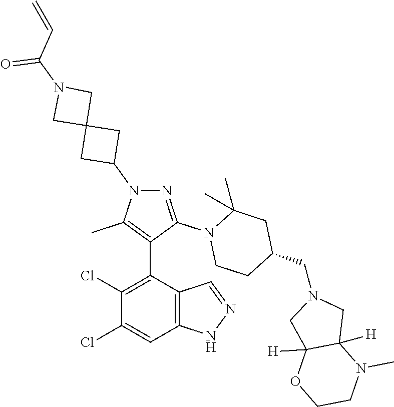 [SMILES: C=CC(=O)N1CC2(CC(n3nc(N4CC[C@@H](CN5CC6OCCN(C)C6C5)CC4(C)C)c(-c4c(Cl)c(Cl)cc5[nH]ncc45)c3C)C2)C1] 1-(6-(4-(5,6-dichloro-1H- indazol-4-yl)-3-((R)-2,2- dimethyl-4-(((4aR*,7aS*)-4- methylhexahydropyrrolo[3,4- b][1,4]oxazin-6(2H)- yl)methyl)piperidin-1-yl)-5- methyl-1H-pyrazol-1-yl)-2- azaspiro[3.3]heptan-2- yl)prop-2-en-1-one | Using Method-13e and Intermediate C118b (Step 2), using Intermediate D6 (Step 3) and RP-HPLC-2 (mobile phase: 5 to 50% CH₃CN in H₂O containing 0.1% TFA); Example 260a = 1st eluting isomer, Example 260b = 2nd eluting isomer. | Example 260b: UPLC-MS-4: Rt = 0.79 min; MS m/z [M + H]⁺ 681.6/683.6/ 685.6, Example 260a: UPLC-MS-4: Rt = 0.73 min; MS m/z [M + H]⁺ 681.6/683.6/ 685.6. |
| 261a/ 261b | 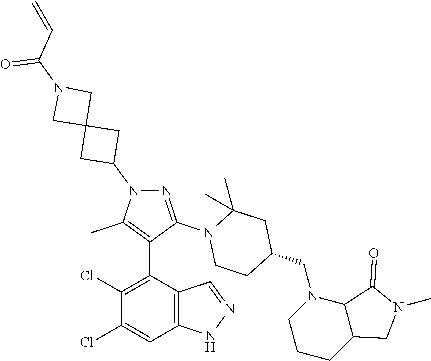 [SMILES: C=CC(=O)N1CC2(CC(n3nc(N4CC[C@@H](CN5CCCC6CN(C)C(=O)C65)CC4(C)C)c(-c4c(Cl)c(Cl)cc5[nH]ncc45)c3C)C2)C1] (4aR*,7aR*)-1-(((R)-1-(1-(2- acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-6- methyloctahydro-7H- pyrrolo[3,4-b]pyridin-7-one | Using Method-13 and Intermediate C123a (Step 3), using Intermediate D6 (Step 3) and RP-HPLC-2 (mobile phase: 5 to 50% CH₃CN in H₂O containing 0.1% TFA); Example 261a = 1st eluting isomer, Example 261b = 2nd eluting isomer. | Example 261b: UPLC-MS-4: Rt = 0.83 min; MS m/z [M + H]⁺ 693.3/695.3/ 697.3, Example 261a: UPLC-MS-4: Rt = 0.78 min; MS m/z [M + H]⁺ 693.3/695.3/ 697.3. |
| 262a/ 262b | 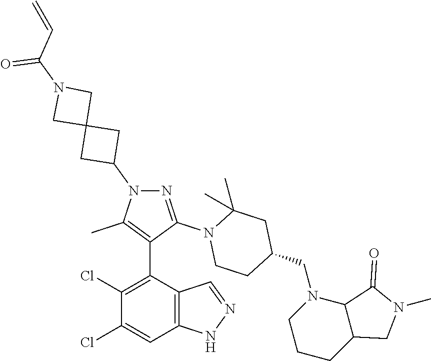 [SMILES: C=CC(=O)N1CC2(CC(n3nc(N4CC[C@@H](CN5CCCC6CN(C)C(=O)C65)CC4(C)C)c(-c4c(Cl)c(Cl)cc5[nH]ncc45)c3C)C2)C1] (4aR*,7aR*)-1-(((R)-1-(1-(2- acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-6- methyloctahydro-7H- pyrrolo[3,4-b]pyridin-7-one | Using Method-13 and Intermediate C123b (Step 3), using Intermediate D6 (Step 3) and reverse phase chromatography (mobile phase: 5 to 50% CH₃CN in H₂O containing 0.1% TFA); Example 262a = 1st eluting isomer, Example 262b = 2nd eluting isomer. | Example 262b: UPLC-MS-4: Rt = 0.81 min; MS m/z [M + H]⁺ 693.3/695.3/ 697.3, Example 262a: UPLC-MS-4: Rt = 0.80 min; MS m/z [M + H]⁺ 693.3/695.3/ 697.3. |
| 263a/ 263b | 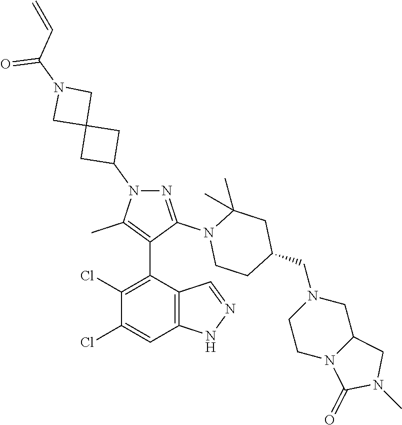 [SMILES: C=CC(=O)N1CC2(CC(n3nc(N4CC[C@@H](CN5CCN6C(=O)N(C)CC6C5)CC4(C)C)c(-c4c(Cl)c(Cl)cc5[nH]ncc45)c3C)C2)C1] 7-(((R)-1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-2- methylhexahydroimidazo[1,5- a]pyrazin-3(2H)-one | Using Method-13 and Intermediate C124a (Step 3), using Intermediate D6 (Step 3) and reverse phase chromatography (mobile phase: 5 to 50% CH₃CN in H₂O containing 0.1% TFA); Example 263a = 1st eluting isomer, Example 263b = 2nd eluting isomer. | Example 263b: UPLC-MS-4: Rt = 0.81 min; MS m/z [M + H]⁺ 694.6/696.6/ 698.6, Example 263a: UPLC-MS-4: Rt = 0.78 min; MS m/z [M + H]⁺ 694.6/696.6/ 698.6. |
| 264a/ 264b | 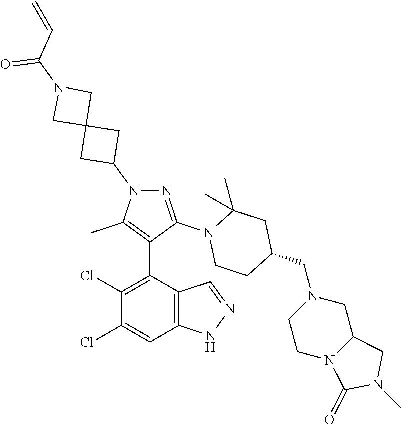 [SMILES: C=CC(=O)N1CC2(CC(n3nc(N4CC[C@@H](CN5CCN6C(=O)N(C)CC6C5)CC4(C)C)c(-c4c(Cl)c(Cl)cc5[nH]ncc45)c3C)C2)C1] 7-(((R)-1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-2- methylhexahydroimidazo[1,5- a]pyrazin-3(2H)-one | Using Method-13 and Intermediate C124b (Step 3), using Intermediate D6 (Step 3) and RP-HPLC-2 (mobile phase: 5 to 50% CH₃CN in H₂O containing 0.1% TFA); Example 264a = 1st eluting isomer, Example 264b = 2nd eluting isomer. | Example 264b: UPLC-MS-4: Rt = 0.81 min; MS m/z [M + H]⁺ 694.6/696.6/ 698.6, Example 264a: UPLC-MS-4: Rt = 0.78 min; MS m/z [M + H]⁺ 694.5/696.5/ 698.5. |
| 265a/ 265b | 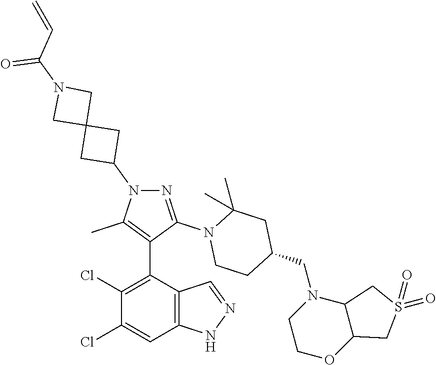 [SMILES: C=CC(=O)N1CC2(CC(n3nc(N4CC[C@@H](CN5CCOC6CS(=O)(=O)CC65)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((4aR*,7aS*)-6,6- dioxidohexahydro-4H- thieno[3,4-b][1,4]oxazin-4- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and Intermediate C125a (Step 3), using Intermediate D6 (Step 3) and reverse phase chromatography (mobile phase: 5 to 50% CH₃CN in H₂O containing 0.1% TFA); Example 265a = 1st eluting isomer, Example 265b = 2nd eluting isomer. | Example 265b: UPLC-MS-4: Rt = 1.01 min; MS m/z [M + H]⁺ 714.5/716.5/ 718.5, Example 265a: UPLC-MS-4: Rt = 0.99 min; MS m/z [M + H]⁺ 714.5/716.5/ 718.5. |
| 266a/ 266b | 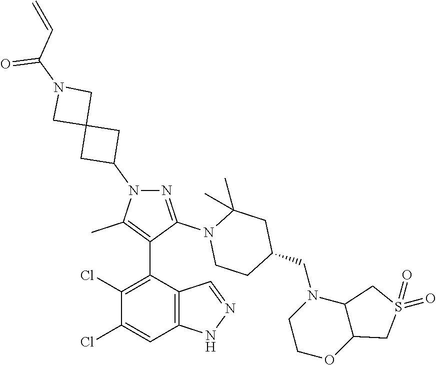 [SMILES: C=CC(=O)N1CC2(CC(n3nc(N4CC[C@@H](CN5CCOC6CS(=O)(=O)CC65)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((4aR*,7aS*)-6,6- dioxidohexahydro-4H- thieno[3,4-b][1,4]oxazin-4- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and Intermediate C125b (Step 3), using Intermediate D6 (Step 3) and reverse phase chromatography (mobile phase: 5 to 50% CH₃CN in H₂O containing 0.1% TFA); Example 266a = 1st eluting isomer, Example 266b = 2nd eluting isomer. | Example 266b: UPLC-MS-4: Rt = 1.01 min; MS m/z [M + H]⁺ 714.5/716.5/ 718.5, Example 266a: UPLC-MS-4: Rt = 0.99 min; MS m/z [M + H]⁺ 714.5/716.5/ 718.5. |
| 267a/ 267b | 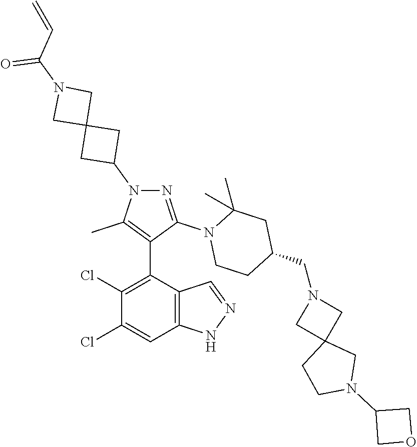 [SMILES: C=CC(=O)N1CC2(CC(n3nc(N4CC[C@@H](CN5CC6(CCN(C7COC7)C6)C5)CC4(C)C)c(-c4c(Cl)c(Cl)cc5[nH]ncc45)c3C)C2)C1] (R)-1-(6-(4-(5,6-dichloro-1H- indazol-4-yl)-3-(2,2-dimethyl- 4-((6-(oxetan-3-yl)-2,6- diazaspiro[3.4]octan-2- yl)methyl)piperidin-1-yl)-5- methyl-1H-pyrazol-1-yl)-2- azaspiro[3.3]heptan-2- yl)prop-2-en-1-one | Using Method-13e and Intermediate A3 (Step 1), using Intermediate D6 (Step 3) and reverse phase chromatography (mobile phase: 5 to 50% CH₃CN in H₂O containing 0.1% TFA); Example 267a = 1st eluting isomer, Example 267b = 2nd eluting isomer. | Example 267b: UPLC-MS-4: Rt = 0.75 min; MS m/z [M + H]⁺ 707.4/709.4/ 711.4, Example 267a: UPLC-MS-4: Rt = 0.65 min; MS m/z [M + H]⁺ 707.4/709.4/ 711.4. |
| 268a/ 268b | 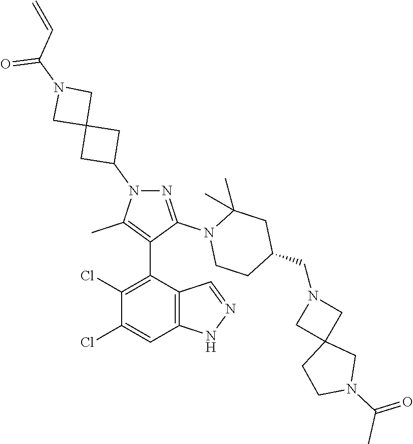 [SMILES: C=CC(=O)N1CC2(CC(n3nc(N4CC[C@@H](CN5CC6(CCN(C(C)=O)C6)C5)CC4(C)C)c(-c4c(Cl)c(Cl)cc5[nH]ncc45)c3C)C2)C1] (R)-1-(6-(3-(4-((6-acetyl-2,6- diazaspiro[3.4]octan-2- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13e and Intermediate A20 (Step 1), using Intermediate D6 (Step 3) and normal phase chromatography (eluent: MeOH in CH₂Cl₂ 0 to 10%) followed by reverse phase chromatography (mobile phase: 5 to 45% CH₃CN in H₂O containing 0.1% TFA); Example 268a = 1st eluting isomer, Example 268b = 2nd eluting isomer. | Example 268b: UPLC-MS-4: Rt = 0.77 min; MS m/z [M + H]⁺ 693.6/695.6/ 697.6, Example 268a: UPLC-MS-4: Rt = 0.73 min; MS m/z [M + H]⁺ 693.6/695.6/ 697.6. |
| 269a/ 269b | 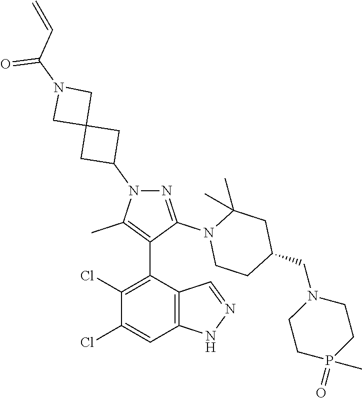 [SMILES: C=CC(=O)N1CC2(CC(n3nc(N4CC[C@@H](CN5CCP(C)(=O)CC5)CC4(C)C)c(-c4c(Cl)c(Cl)cc5[nH]ncc45)c3C)C2)C1] (R)-1-(6-(4-(5,6-dichloro-1H- indazol-4-yl)-3-(2,2-dimethyl- 4-((4-methyl-4-oxido-1,4- azaphosphinan-1- yl)methyl)piperidin-1-yl)-5- methyl-1H-pyrazol-1-yl)-2- azaspiro[3.3]heptan-2- yl)prop-2-en-1-one | Using Method-13d and CAS [945460-43-5] (Step 1), using Intermediate D6 (Step 3) and RP-HPLC-2 (mobile phase: 5 to 80% CH₃CN in H₂O containing 0.1% TFA); Example 269a = 1st eluting isomer, Example 269b = 2nd eluting isomer. | Example 269b: UPLC-MS-4: Rt = 0.78 min; MS m/z [M + H]⁺ 672.3/674.3/ 676.3, Example 269a: UPLC-MS-4: Rt = 0.73 min; MS m/z [M + H]⁺ 672.3/674.3/ 676.3. |
| 270a/ 270b | 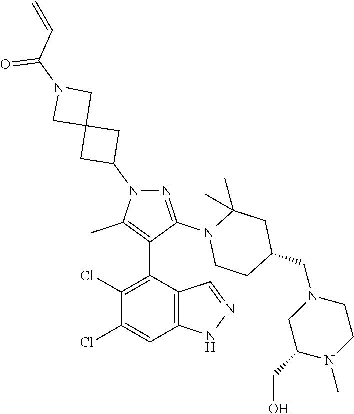 [SMILES: C=CC(=O)N1CC2(CC(n3nc(N4CC[C@@H](CN5CCN(C)[C@H](CO)C5)CC4(C)C)c(-c4c(Cl)c(Cl)cc5[nH]ncc45)c3C)C2)C1] 1-(6-(4-(5,6-dichloro-1H- indazol-4-yl)-3-((R)-4-(((S)-3- (hydroxymethyl)-4- methylpiperazin-1-yl)methyl)- 2,2-dimethylpiperidin-1-yl)-5- methyl-1H-pyrazol-1-yl)-2- azaspiro[3.3]heptan-2- yl)prop-2-en-1-one | Using Method-13ad and (S)-(1-methylpiperazin-2- yl)methanol (Step 1), using Intermediate D6 (Step 3) and RP-HPLC-2 (mobile phase: 10 to 50% CH₃CN in H₂O containing 0.1% TFA); Example 270a = 1st eluting isomer, Example 270b = 2nd eluting isomer. | Example 270b: UPLC-MS-4: Rt = 0.81 min; MS m/z [M + H]⁺ 669.6/671.6/ 673.6, Example 270a: UPLC-MS-4: Rt = 0.75 min; MS m/z [M + H]⁺ 669.6/671.6/ 673.6. |
| 271a/ 271b | 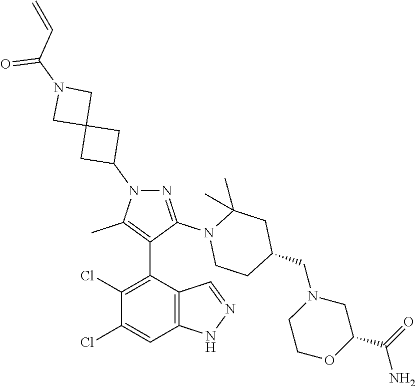 [SMILES: C=CC(=O)N1CC2(CC(n3nc(N4CC[C@@H](CN5CCO[C@@H](C(N)=O)C5)CC4(C)C)c(-c4c(Cl)c(Cl)cc5[nH]ncc45)c3C)C2)C1] (R)-4-(((R)-1-(1-(2-acryloyl- 2-azaspiro[3.3]heptan-6-yl)- 4-(5,6-dichloro-1H-indazol-4- yl)-5-methyl-1H-pyrazol-3- yl)-2,2-dimethylpiperidin-4- yl)methyl)morpholine-2- carboxamide | Using Method-13ad and Intermediate A21 (Step 1), using Intermediate D6 (Step 3) and RP-HPLC-2 (mobile phase: 10 to 40% CH₃CN in H₂O containing 0.1% TFA); Example 271a = 1st eluting isomer, Example 271b = 2nd eluting isomer. | Example 271b: UPLC-MS-4: Rt = 0.82 min; MS m/z [M + H]⁺ 669.4/671.4/ 673.4, Example 271a: UPLC-MS-4: Rt = 0.76 min; MS m/z [M + H]⁺ 669.4/671.4/ 673.4. |
| 272a/ 272b | 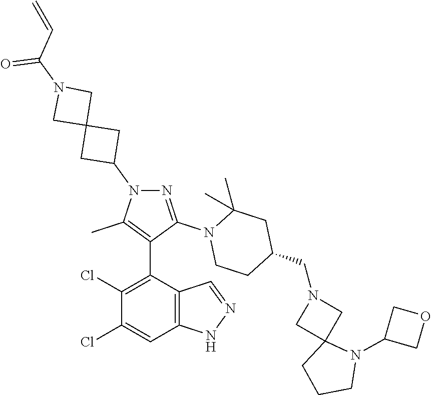 [SMILES: C=CC(=O)N1CC2(CC(n3nc(N4CC[C@@H](CN5CC6(CCCN6C6COC6)C5)CC4(C)C)c(-c4c(Cl)c(Cl)cc5[nH]ncc45)c3C)C2)C1] (R)-1-(6-(4-(5,6-dichloro-1H- indazol-4-yl)-3-(2,2-dimethyl- 4-((5-(oxetan-3-yl)-2,5- diazaspiro[3.4]octan-2- yl)methyl)piperidin-1-yl)-5- methyl-1H-pyrazol-1-yl)-2- azaspiro[3.3]heptan-2- yl)prop-2-en-1-one | Using Method-13ad and Intermediate A4 (Step 1), using Intermediate D6 (Step 3) and RP-HPLC-2 (mobile phase: 5 to 45% CH₃CN in H₂O containing 0.1% TFA); Example 2702 = 1st eluting isomer, Example 272b = 2nd eluting isomer. | Example 272b: UPLC-MS-4: Rt = 0.82 min; MS m/z [M + H]⁺ 707.3/705.3/ 707.3, Example 272a: UPLC-MS-4: Rt = 0.78 min; MS m/z [M + H]⁺ 707.3/705.3/ 707.3. |
| 273a/ 273b | 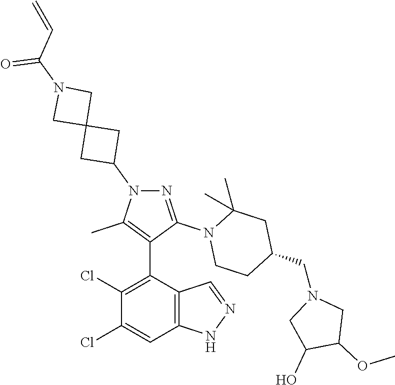 [SMILES: C=CC(=O)N1CC2(CC(n3nc(N4CC[C@@H](CN5CC(O)C(OC)C5)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((3S*,4S*)-3-hydroxy-4- methoxypyrrolidin-1- yl)methyl)-2,2- dimethylpiperidin-1-yl)-5- methyl-1H-pyrazol-1-yl)-2- azaspiro[3.3]heptan-2- yl)prop-2-en-1-one | Using Method-13d and Intermediate C126a (Step 3) and Intermediate D6 (Step 3) and RP-HPLC-2 (mobile phase: 10 to 40% CH₃CN in H₂O containing 0.1% TFA); Example 273a = 1st eluting isomer, Example 273b = 2nd eluting isomer. | Example 273b: UPLC-MS-4: Rt = 0.82 min; MS m/z [M + H]⁺ 656.3/658.3/ 660.3, Example 273a: UPLC-MS-4: Rt = 0.76 min; MS m/z [M + H]⁺ 656.3/658.3/ 660.3. |
| 274a/ 274b | 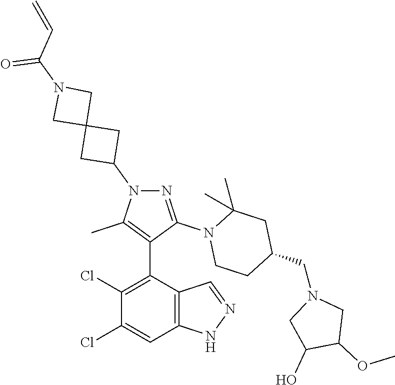 [SMILES: C=CC(=O)N1CC2(CC(n3nc(N4CC[C@@H](CN5CC(O)C(OC)C5)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((3S*,4S*)-3-hydroxy-4- methoxypyrrolidin-1- yl)methyl)-2,2- dimethylpiperidin-1-yl)-5- methyl-1H-pyrazol-1-yl)-2- azaspiro[3.3]heptan-2- yl)prop-2-en-1-one | Using Method-13d and Intermediate C126b (Step 3) and Intermediate D6 (Step 3) and RP-HPLC-2 (mobile phase: 10 to 40% CH₃CN in H₂O containing 0.1% TFA); Example 274a = 1st eluting isomer, Example 274b = 2nd eluting isomer. | Example 274b: UPLC-MS-4: Rt = 0.79 min; MS m/z [M + H]⁺ 656.3/658.3/ 660.3, Example 274a: UPLC-MS-4: Rt = 0.76 min; MS m/z [M + H]⁺ 656.3/658.3/ 660.3. |
| 275a/ 275b | 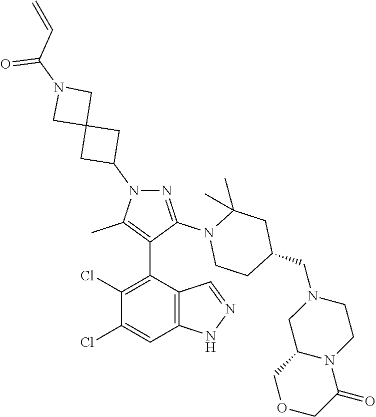 [SMILES: C=CC(=O)N1CC2(CC(n3nc(N4CC[C@@H](CN5CCN6C(=O)COC[C@@H]6C5)CC4(C)C)c(-c4c(Cl)c(Cl)cc5[nH]ncc45)c3C)C2)C1] (S)-8-(((R)-1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)hexahydropyrazino [2, 1-c][1,4]oxazin-4(3H)-one | Using Method-13ad and (S)- hexahydropyrazino[2,1- C][1,4]oxazin-4(3H)-one hydrochloride (CAS [1383427-98-2]) (Step 1), using Intermediate D6 (Step 3) and RP-HPLC-2 (mobile phase: 10 to 40% CH₃CN in H₂O containing 0.1% TFA); Example 275a = 1st eluting isomer, Example 275b = 2nd eluting isomer. | Example 275b: ¹H NMR (400 MHz, DMSO-d₆) δ 13.2 (s, 1H), 7.78 (s, 1H), 7.63 (s, 1H), 6.30 (m, 1H), 6.09 (m, 1H), 5.67 (m, 1H), 4.76 (m, 1H), 4.34 (s, 1H), 4.28 (s, 1H), 4.27 (m, 1H), 4.05 (s, 1H), 4.00 (s, 1H), 3.98 (s, 2H), 3.93 (dd, 1H), 3.53-3.41 (m, 2H), 3.17 (m, 1H), 2.84-2.57 (m, 8H), 2.01 (s, 3H), 1.96 (m, 2H), 1.82-1.69 (m, 2H), 1.65 (t, 1H), 1.57 (m, 1H), 1.34 (m, 1H), 1.14 (s, 3H), 0.73 (m, 1.5H), 0.71 (s, 1.5H), 0.74-0.57 (m, 2H); UPLC-MS-4: Rt 0.83 min; MS m/z [M + H]⁺ 695.5/697.5/ 699.5, Example 275a: UPLC-MS-4: Rt = 0.80 min; MS m/z [M + H]⁺ 695.5/697.5/ 699.5. |
| 276a/ 276b | 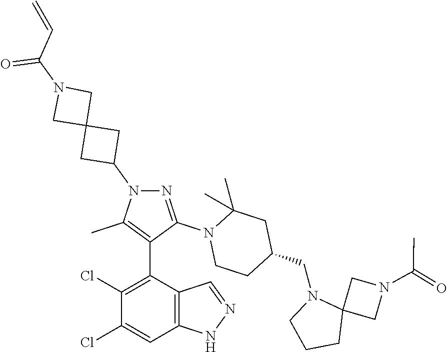 [SMILES: C=CC(=O)N1CC2(CC(n3nc(N4CC[C@@H](CN5CCCC56CN(C(C)=O)C6)CC4(C)C)c(-c4c(Cl)c(Cl)cc5[nH]ncc45)c3C)C2)C1] (R)-1-(6-(3-(4-((2-acetyl-2,5- diazaspiro[3.4]octan-5- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13ad and Intermediate A22 (Step 1), using Intermediate D6 (Step 3) and RP-HPLC-2 (mobile phase: 10 to 40% CH₃CN in H₂O containing 0.1% TFA); Example 276a = 1st eluting isomer, Example 276b = 2nd eluting isomer. | Example 276b: UPLC-MS-2e: Rt = 4.07 min; MS m/z [M + H]⁺ 693.3/695.3/ 697.3, Example 276a: UPLC-MS-2e: Rt = 3.92 min; MS m/z [M + H]⁺ 693.3/695.3/ 697.3. |
| 277a/ 277b | 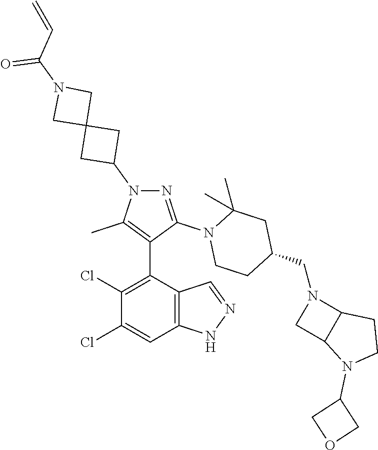 [SMILES: C=CC(=O)N1CC2(CC(n3nc(N4CC[C@@H](CN5CC6C5CCN6C5COC5)CC4(C)C)c(-c4c(Cl)c(Cl)cc5[nH]ncc45)c3C)C2)C1] 1-(6-(4-(5,6-dichloro-1H- indazol-4-yl)-3-((R)-2,2- dimethyl-4-(((1S*,5S*)-2- (oxetan-3-yl)-2,6- diazabicyclo[3.2.0]heptan-6- yl)methyl)piperidin-1-yl)-5- methyl-1H-pyrazol-1-yl)-2- azaspiro[3.3]heptan-2- yl)prop-2-en-1-one | Using Method-13d and Intermediate C127a (Step 3), using Intermediate D6 (Step 3) and RP-HPLC-2 (mobile phase: 10 to 40% CH₃CN in H₂O containing 0.1% TFA); Example 277a = 1st eluting isomer, Example 277b = 2nd eluting isomer. | Example 277b: UPLC-MS-4: Rt = 0.81 min; MS m/z [M + H]⁺ 693.5/695.5/ 697.5, Example 277a: UPLC-MS-4: Rt = 0.76 min; MS m/z [M + H]⁺ 693.5/695.5/ 697.5. |
| 278a/ 278b | 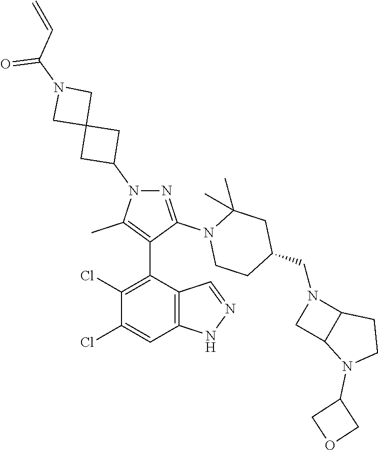 [SMILES: C=CC(=O)N1CC2(CC(n3nc(N4CC[C@@H](CN5CC6C5CCN6C5COC5)CC4(C)C)c(-c4c(Cl)c(Cl)cc5[nH]ncc45)c3C)C2)C1] 1-(6-(4-(5,6-dichloro-1H- indazol-4-yl)-3-((R)-2,2- dimethyl-4-(((1S*,5S*)-2- (oxetan-3-yl)-2,6- diazabicyclo[3.2.0]heptan-6- yl)methyl)piperidin-1-yl)-5- methyl-1H-pyrazol-1-yl)-2- azaspiro[3.3]heptan-2- yl)prop-2-en-1-one | Using Method-13d and Intermediate C127b (Step 3), using Intermediate D6 (Step 3) and RP-HPLC-2 (mobile phase: 10 to 40% CH₃CN in H₂O containing 0.1% TFA); Example 278a = 1st eluting isomer, Example 278b = 2nd eluting isomer. | Example 278b: UPLC-MS-4: Rt = 0.78 min; MS m/z [M + H]⁺ 693.6/695.6/ 697.6, Example 278a: UPLC-MS-4: Rt = 0.74 min; MS m/z [M + H]⁺ 693.6/695.6/ 697.6. |
| 279a/ 279b | 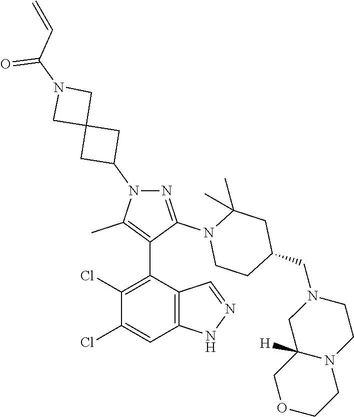 1-(6-(4-(5,6-dichloro-1H- indazol-4-yl)-3-((R)-4-(((S)- hexahydropyrazino[2,1- c][1,4]oxazin-8(1H)- yl)methyl)-2,2- dimethylpiperidin-1-yl)-5- methyl-1H-pyrazol-1-yl)-2- azaspiro[3.3]heptan-2- yl)prop-2-en-1-one | Using Method-13ad and Intermediate (S)- octahydropyrazino[2,1- c][1,4]oxazine (Step 1), using Intermediate D6 (Step 3) and RP-HPLC-2 (mobile phase: 5 to 40% CH₃CN in H₂O containing 0.1% TFA); Example 279a = 1st eluting isomer, Example 279b = 2nd eluting isomer. | Example 279b: ¹H NMR (400 MHz, DMSO-d₆) δ 13.1 (s, 1H), 7.77 (s, 1H), 7.63 (s, 1H), 6.30 (m, 1H), 6.09 (m, 1H), 5.67 (m, 1H), 4.75 (m, 1H), 4.33 (s, 1H), 4.28 (s, 1H), 4.05 (s, 1H), 4.00 (s, 1H), 3.69 (m, 1H), 3.54 (m, 1H), 3.44 (m, 1H), 3.15 (m, 2H), 3.02 (t, 1H), 2.83-2.62 (m, 6H), 2.61-2.44 (m, 2H), 2.16-1.99 (m, 3H), 2.01 (s, 3H), 1.96-1.87 (m, 3H), 1.74 (m, 1H), 1.57- 1.46 (m, 2H), 1.31 (m, 1H), 1.12 (s, 3H), 0.71 (m, 1.5H), 0.69 (s, 1.5H), 0.71-0.63 (m, 1H), 0.58 (m, 1H); UPLC-MS-4: Rt = 0.81 min; MS m/z [M + H]⁺ 681.6/683.6/ 685.6, Example 279a: UPLC-MS-4: Rt = 0.76 min; MS m/z [M + H]⁺ 681.6/683.6/ 685.6. |
| 280a/ 280b | 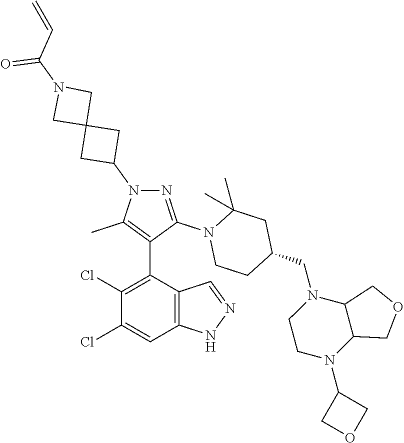 [SMILES: C=CC(=O)N1CC2(CC(n3nc(N4CC[C@@H](CN5CCN(C6COC6)C6COCC65)CC4(C)C)c(-c4c(Cl)c(Cl)cc5[nH]ncc45)c3C)C2)C1] 1-(6-(4-(5,6-dichloro-1H- indazol-4-yl)-3-((R)-2,2- dimethyl-4-(((4aS*,7aR*)-4- (oxetan-3- yl)hexahydrofuro[3,4- b]pyrazin-1(2H)- yl)methyl)piperidin-1-yl)-5- methyl-1H-pyrazol-1-yl)-2- azaspiro[3.3]heptan-2- yl)prop-2-en-1-one | Using Method-13ad and Intermediate C122 (Step 2), using Intermediate D6 (Step 3) and RP-HPLC-2 (mobile phase: 5 to 40% CH₃CN in H₂O containing 0.1% TFA); Example 280a = 1st eluting isomer, Example 280b = 2nd eluting isomer. | Example 280b: UPLC-MS-4: Rt = 0.89 min; MS m/z [M + H]⁺ 723.7/725.7/ 727.7, Example 280a: UPLC-MS-4: Rt = 0.85 min; MS m/z [M + H]⁺ 723.7/725.7/ 727.7. |
| 281a/ 281b | 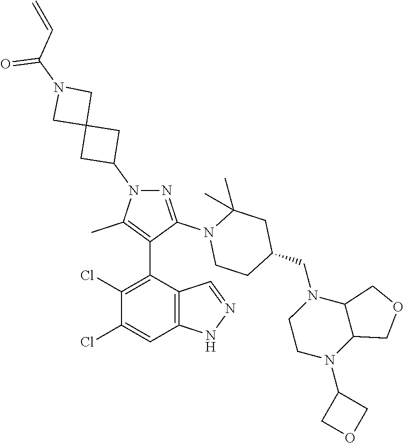 [SMILES: C=CC(=O)N1CC2(CC(n3nc(N4CC[C@@H](CN5CCN(C6COC6)C6COCC65)CC4(C)C)c(-c4c(Cl)c(Cl)cc5[nH]ncc45)c3C)C2)C1] 1-(6-(4-(5,6-dichloro-1H- indazol-4-yl)-3-((R)-2,2- dimethyl-4-(((4aS*,7aR*)-4- (oxetan-3- yl)hexahydrofuro[3,4- b]pyrazin-1(2H)- yl)methyl)piperidin-1-yl)-5- methyl-1H-pyrazol-1-yl)-2- azaspiro[3.3]heptan-2- yl)prop-2-en-1-one | Using Method-13ad and Intermediate C121 (Step 2), using Intermediate D6 (Step 3) and RP-HPLC-2 (mobile phase: 10 to 45% CH₃CN in H₂O containing 0.1% TFA); Example 281a = 1st eluting isomer, Example 281b = 2nd eluting isomer. | Example 281b: UPLC-MS-4: Rt = 0.87 min; MS m/z [M + H]⁺ 723.8/725.8/ 727.8, Example 281a: UPLC-MS-4: Rt = 0.86 min; MS m/z [M + H]⁺ 723.8/725.8/ 727.8. |
| 282a/ 282b | 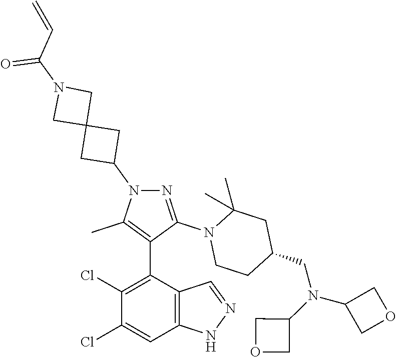 [SMILES: C=CC(=O)N1CC2(CC(n3nc(N4CC[C@@H](CN(C5COC5)C5COC5)CC4(C)C)c(-c4c(Cl)c(Cl)cc5[nH]ncc45)c3C)C2)C1] (R)-1-(6-(3-(4-((di(oxetan-3- yl)amino)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13 and di(oxetan-3-yl)amine (Step 1), using Intermediate D6 (Step 3) and C-SFC-4 (mobile phase: CO₂/[IPA + 0.1% Et₃N 70/30); Example 282a = 1st eluting isomer, Example 282b = 2nd eluting isomer. | Example 282b: UPLC-MS-2e: Rt = 4.96 min; MS m/z [M + H]⁺ 668.3/670.3/ 672.3; C-SFC-3 (mobile phase: CO₂/[IPA + 0.025% NH₃] 70/30): Rt = 2.49 min, Example 282a: C-SFC-3 (mobile phase: CO₂/[IPA + 0.025% NH₃] 70/30): Rt = 1.75 min. |
| 283a/ 283b | 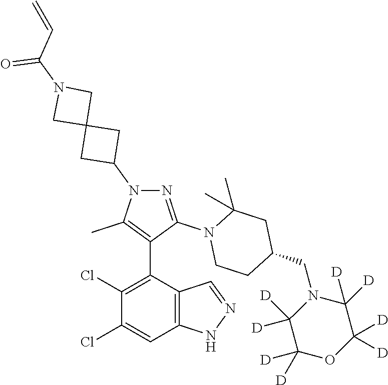 [SMILES: [2H]C1([2H])OC([2H])([2H])C([2H])([2H])N(C[C@@H]2CCN(c3nn(C4CC5(C4)CN(C(=O)C=C)C5)c(C)c3-c3c(Cl)c(Cl)cc4[nH]ncc34)C(C)(C)C2)C1([2H])[2H]] (R)-1-(6-(4-(5,6-dichloro-1H- indazol-4-yl)-3-(2,2-dimethyl- 4-((morpholino- d8)methyl)piperidin-1-yl)-5- methyl-1H-pyrazol-1-yl)-2- azaspiro[3.3]heptan-2- yl)prop-2-en-1-one | Using Method-13 and Morpholine-2,2,3,3,5,5,6,6- d₈ (Step 1), using Intermediate D6 (Step 3) and RP-HPLC-2 (mobile phase: 10 to 40% CH₃CN in H₂O containing 0.1% TFA); Example 283a = 1st eluting isomer, Example 283b = 2nd eluting isomer. | Example 283b: UPLC-MS-2e: Rt = 3.84 min; MS m/z [M + H]⁺ 634.3/636.3/ 638.3; C-SFC-3 (mobile phase: CO₂/[IPA + 0.012% NH₃] 70/30): Rt = 1.91 min, Example 283a: C-SFC-3 (mobile phase: CO₂/[IPA + 0.012% NH₃] 70/30): Rt = 1.24 min. |
| 284a/ 284b | 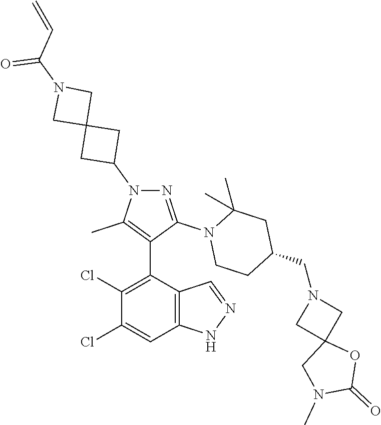 [SMILES: C=CC(=O)N1CC2(CC(n3nc(N4CC[C@@H](CN5CC6(C5)CN(C)C(=O)O6)CC4(C)C)c(-c4c(Cl)c(Cl)cc5[nH]ncc45)c3C)C2)C1] (R)-2-((1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-7-methyl-5-oxa- 2,7-diazaspiro[3.4]octan-6- one | Using Method-13 and 7- methyl-5-oxa-2,7- diazaspiro[3.4]octan-6-one hydrochloride (Step 1), using Intermediate D6 (Step 3) and RP-HPLC-2 (mobile phase: 10 to 40% CH₃CN in H₂O containing 0.1% TFA); Example 284a = 1st eluting isomer, Example 284b = 2nd eluting isomer. | Example 284b: UPLC-MS-2e: Rt = 3.83 min; MS m/z [M + H]⁺ 681.3/683.3/ 685.3, Example 284a: UPLC-MS-2e: Rt = 3.53 min; MS m/z [M + H]⁺ 681.3/683.3/ 685.3. |
| 285a/ 285b | 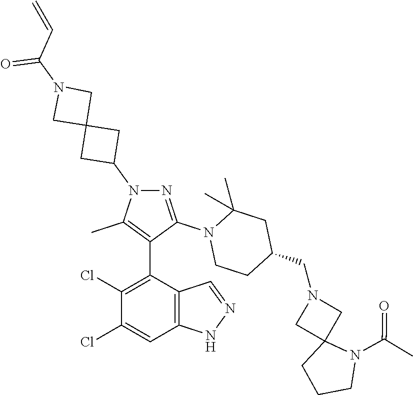 [SMILES: C=CC(=O)N1CC2(CC(n3nc(N4CC[C@@H](CN5CC6(CCCN6C(C)=O)C5)CC4(C)C)c(-c4c(Cl)c(Cl)cc5[nH]ncc45)c3C)C2)C1] (R)-1-(6-(3-(4-((5-acetyl-2,5- diazaspiro[3.4]octan-2- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13ad and Intermediate A23 (Step 1), using Intermediate D6 (Step 3) and RP-HPLC-2 (mobile phase: 10 to 40% CH₃CN in H₂O containing 0.1% TFA); Example 285a = 1st eluting isomer, Example 285b = 2nd eluting isomer. | Example 285b: UPLC-MS-2e: Rt = 3.94 min; MS m/z [M + H]⁺ 693.3/695.3/ 697.3, Example 285a: UPLC-MS-2e: Rt = 3.62 min; MS m/z [M + H]⁺ 693.3/695.3/ 697.3. |
| 286a/ 286b | 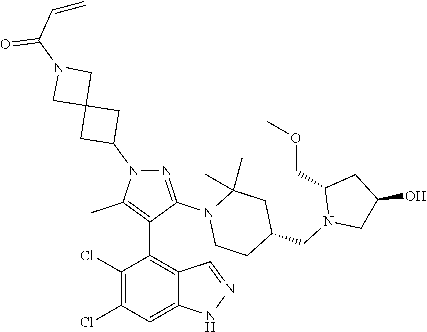 [SMILES: C=CC(=O)N1CC2(CC(n3nc(N4CC[C@@H](CN5C[C@H](O)C[C@H]5COC)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((2S,4R)-4-hydroxy-2- (methoxymethyl)pyrrolidin-1- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and (3R,5S)-5- (methoxymethyl)pyrrolidin- 3-ol (Step 1), using Intermediate D6 (Step 3) and normal phase chromatography (eluent: MeOH in CH₂Cl₂ from 0 to 20%); Example 286a = 1st eluting isomer, Example 286b = 2nd eluting isomer. | Example 286b: UPLC-MS-2e: Rt = 3.98 min; MS m/z [M + H]⁺ 670.4/672.4/ 674.4, Example 286a: UPLC-MS-2e: Rt = 3.66 min; MS m/z [M + H]⁺ 670.4/672.4/ 674.4. |
| 287a/ 287b | 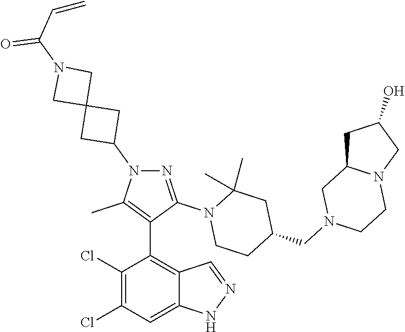 [SMILES: C=CC(=O)N1CC2(CC(n3nc(N4CC[C@@H](CN5CCN6C[C@@H](O)C[C@@H]6C5)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((7S,8aR)-7- hydroxyhexahydropyrrolo[1,2- a]pyrazin-2(1H)-yl)methyl)- 2,2-dimethylpiperidin-1-yl)-5- methyl-1H-pyrazol-1-yl)-2- azaspiro[3.3]heptan-2- yl)prop-2-en-1-one | Using Method-13 and (7S,8aR)- octahydropyrrolo[1,2- a]piperazin-7-ol oxalic acid (Step 1), using Intermediate D6 (Step 3) and C-SFC-4 (mobile phase: CO₂/[IPA + 0.05% Et₃N] 68/32); Example 287a = 1st eluting isomer, Example 287b = 2nd eluting isomer. | Example 287b: UPLC-MS-2e: Rt = 3.96 min; MS m/z [M + H]⁺ 681.3/683.3/ 685.3; C-SFC-3 (mobile phase: CO₂/[IPA + 0.012% NH₃] 68/32): Rt = 3.07 min, Example 287a: C-SFC-3 (mobile phase: CO₂/[IPA + 0.012% NH₃] 68/32): Rt = 1.71 min. |
| 288a/ 288b | 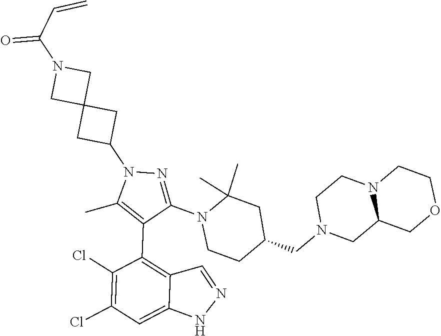 [SMILES: C=CC(=O)N1CC2(CC(n3nc(N4CC[C@@H](CN5CCN6CCOC[C@H]6C5)CC4(C)C)c(-c4c(Cl)c(Cl)cc5[nH]ncc45)c3C)C2)C1] 1-(6-(4-(5,6-dichloro-1H- indazol-4-yl)-3-((R)-4-(((R)- hexahydropyrazino[2,1- c][1,4]oxazin-8(1H)- yl)methyl)-2,2- dimethylpiperidin-1-yl)-5- methyl-1H-pyrazol-1-yl)-2- azaspiro[3.3]heptan-2- yl)prop-2-en-1-one | Using Method-13d and (R)- octahydropyrazino[2,1- c][1,4]oxazine (Step 1), using Intermediate D6 (Step 3) and normal phase chromatography (eluent: MeOH in CH₂Cl₂ from 0 to 10%); Example 288a = 1st eluting isomer, Example 288b = 2nd eluting isomer. | Example 288b: UPLC-MS-4: Rt = 0.78 min; MS m/z [M + H]⁺ 681.5/683.5/ 685.5, Example 288a: UPLC-MS-4: Rt = 0.75 min; MS m/z [M + H]⁺ 681.5/683.5/ 685.5. |
| 289a/ 289b | 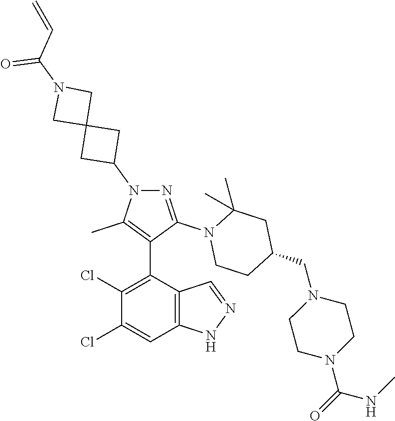 [SMILES: C=CC(=O)N1CC2(CC(n3nc(N4CC[C@@H](CN5CCN(C(=O)NC)CC5)CC4(C)C)c(-c4c(Cl)c(Cl)cc5[nH]ncc45)c3C)C2)C1] (R)-4-((1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-N- methylpiperazine-1- carboxamide | Using Method-13ad and CAS [163361-25-9] (Step 1), using Intermediate D6 (Step 3) and RP-HPLC-2 (mobile phase: 5 to 40% CH₃CN in H₂O containing 0.1% TFA); Example 289a = 1st eluting isomer, Example 289b = 2nd eluting isomer. | Example 289b: UPLC-MS-4: Rt = 0.80 min; MS m/z [M + H]⁺ 682.6/684.6/ 686.6, Example 289a: UPLC-MS-4: Rt = 0.76 min; MS m/z [M + H]⁺ 682.6/684.6/ 686.6. |
| 290a/ 290b | 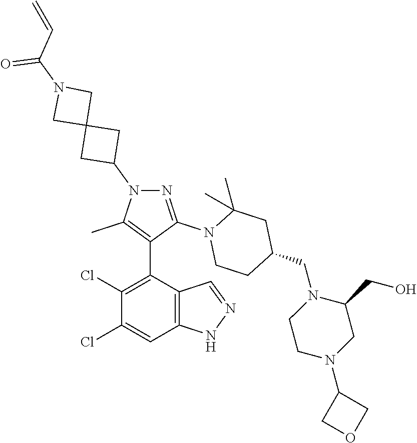 [SMILES: C=CC(=O)N1CC2(CC(n3nc(N4CC[C@@H](CN5CCN(C6COC6)C[C@@H]5CO)CC4(C)C)c(-c4c(Cl)c(Cl)cc5[nH]ncc45)c3C)C2)C1] 1-(6-(4-(5,6-dichloro-1H- indazol-4-yl)-3-((R)-4-(((R)-2- (hydroxymethyl)-4-(oxetan-3- yl)piperazin-1-yl)methyl)-2,2- dimethylpiperidin-1-yl)-5- methyl-1H-pyrazol-1-yl)-2- azaspiro[3.3]heptan-2- yl)prop-2-en-1-one | Using Method-13 and Intermediate A7 (Step 1), using Intermediate D6 (Step 3) and RP-HPLC-2 (mobile phase: 5 to 35% CH₃CN in H₂O containing 0.1% TFA); Example 290a = 1st eluting isomer, Example 290b = 2nd eluting isomer. | Example 290b: UPLC-MS-4: Rt = 0.78 min; MS m/z [M + H]⁺ 711.4/713.4/ 715.4, Example 290a: UPLC-MS-4: Rt = 0.75 min; MS m/z [M + H]⁺ 711.4/713.4/ 715.4. |
| 291a/ 291b | 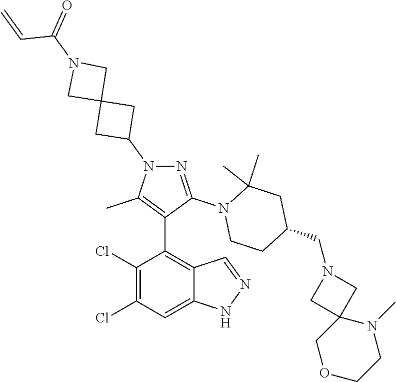 [SMILES: C=CC(=O)N1CC2(CC(n3nc(N4CC[C@@H](CN5CC6(COCCN6C)C5)CC4(C)C)c(-c4c(Cl)c(Cl)cc5[nH]ncc45)c3C)C2)C1] (R)-1-(6-(4-(5,6-dichloro-1H- indazol-4-yl)-3-(2,2-dimethyl- 4-((5-methyl-8-oxa-2,5- diazaspiro[3.5]nonan-2- yl)methyl)piperidin-1-yl)-5- methyl-1H-pyrazol-1-yl)-2- azaspiro[3.3]heptan-2- yl)prop-2-en-1-one | Using Method-13 and Intermediate A8 (Step 1), using Intermediate D6 (Step 3) and RP-HPLC-2 (mobile phase: 5 to 40% CH₃CN in H₂O containing 0.1% TFA); Example 291a = 1st eluting isomer, Example 291b = 2nd eluting isomer. | Example 291b: UPLC-MS-2e: Rt = 3.80 min; MS m/z [M + H]⁺ 681.3/683.3/ 685.3, Example 291a: UPLC-MS-2e: Rt = 3.50 min; MS m/z [M + H]⁺ 681.3/683.3/ 685.3. |
| 292a/ 292b | 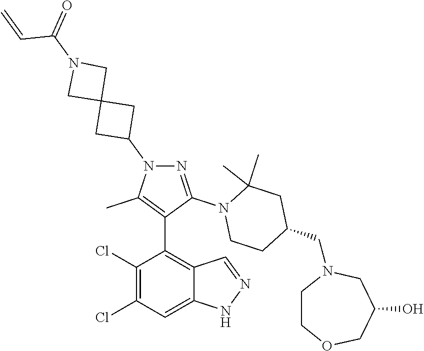 [SMILES: C=CC(=O)N1CC2(CC(n3nc(N4CC[C@@H](CN5CCOC[C@@H](O)C5)CC4(C)C)c(-c4c(Cl)c(Cl)cc5[nH]ncc45)c3C)C2)C1] 1-(6-(4-(5,6-dichloro-1H- indazol-4-yl)-3-((R)-4-(((S)-6- hydroxy-1,4-oxazepan-4- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and (S)- 1,4-oxazepan-6-ol (CAS [1373232-31-5]) (Step 1), using Intermediate D6 (Step 3) and RP-HPLC-2 (mobile phase: 8 to 48% CH₃CN in H₂O containing 0.1% TFA); Example 292a = 1st eluting isomer, Example 292b = 2nd eluting isomer. | Example 292b: UPLC-MS-4: Rt = 0.79 min; MS m/z [M + H]⁺ 656.3/658.4/ 660.3, Example 292a: UPLC-MS-4: Rt = 0.72 min; MS m/z [M + H]⁺ 656.3/658.2/ 660.3. |
| 293a/ 293b | 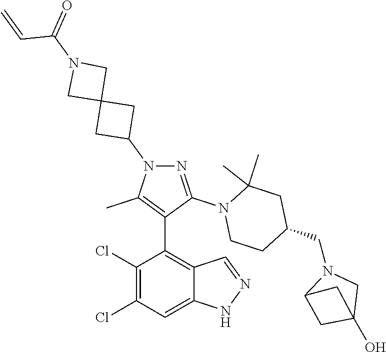 [SMILES: C=CC(=O)N1CC2(CC(n3nc(N4CC[C@@H](CN5CC6(O)CC5C6)CC4(C)C)c(-c4c(Cl)c(Cl)cc5[nH]ncc45)c3C)C2)C1] (R)-1-(6-(4-(5,6-dichloro-1H- indazol-4-yl)-3-(4-((4- hydroxy-2- azabicyclo[2.1.1]hexan-2- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and Intermediate A24 (Step 1), using Intermediate D6 (Step 3) and RP-HPLC-2 (mobile phase: 10 to 40% CH₃CN in H₂O containing 0.1% TFA); Example 293a = 1st eluting isomer, Example 293b = 2nd eluting isomer. | Example 293b: UPLC-MS-4: Rt = 0.78 min; MS m/z [M + H]⁺ 638.4/640.4/ 642.4, Example 293a: UPLC-MS-4: Rt = 0.73 min; MS m/z [M + H]⁺ 638.4/640.4/ 642.4. |
| 294a/ 294b | 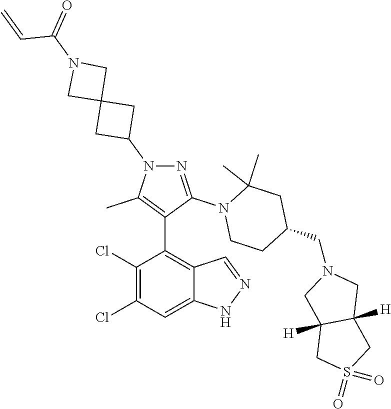 [SMILES: C=CC(=O)N1CC2(CC(n3nc(N4CC[C@@H](CN5C[C@@H]6CS(=O)(=O)C[C@@H]6C5)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((3aR,6aS)-2,2- dioxidotetrahydro-1H- thieno[3,4-c]pyrrol-5(3H)- yl)methyl)-2,2- dimethylpiperidin-1-yl)-5- methyl-1H-pyrazol-1-yl)-2- azaspiro[3.3]heptan-2- yl)prop-2-en-1-one | Using Method-13 and (3aR,6aS)-hexahydro-1H- thieno[3,4-c]pyrrole 2,2- dioxide hydrochloride (CAS [2137033-01-1]) (Step 1), using Intermediate D6 (Step 3) and RP-HPLC-2 (mobile phase: 10 to 40% CH₃CN in H₂O containing 0.1% TFA); Example 294a = 1st eluting isomer, Example 294b = 2nd eluting isomer. | Example 294b: UPLC-MS-2e: Rt = 3.84 min; MS m/z [M + H]⁺ 700.3/702.2/ 704.3, Example 294a: UPLC-MS-2e: Rt = 3.53 min; MS m/z [M + H]⁺ 700.3/702.2/ 704.6. |
| 295a/ 295b | 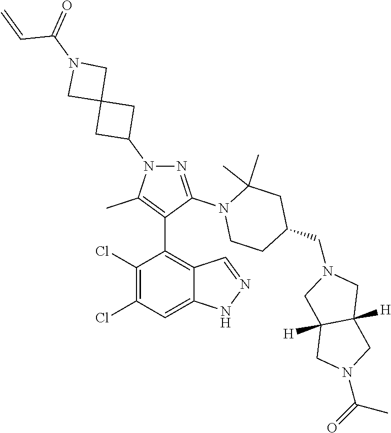 [SMILES: C=CC(=O)N1CC2(CC(n3nc(N4CC[C@@H](CN5C[C@@H]6CN(C(C)=O)C[C@@H]6C5)CC4(C)C)c(-c4c(Cl)c(Cl)cc5[nH]ncc45)c3C)C2)C1] 1-(6-(3-((R)-4-(((3aR,6aS)-5- acetylhexahydropyrrolo[3,4- c]pyrrol-2(1H)-yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13 and 1- ((3aR,6aS)- hexahydropyrrolo[3,4- c]pyrrol-2(1H)-yl)ethan-1- one hydrochloride (CAS [2126144-11-2]) (Step 1), using Intermediate D6 (Step 3) and and RP- HPLC-2 (mobile phase: 10 to 40% CH₃CN in H₂O containing 0.1% TFA) Example 295a = 1st eluting isomer, Example 295b = 2nd eluting isomer. | Example 295b: UPLC-MS-4: Rt = 0.77 min; MS m/z [M + H]⁺ 693.4/695.5/ 697.5, Example 295a: UPLC-MS-4: Rt = 0.74 min; MS m/z [M + H]⁺ 693.4/695.4/ 697.4. |
| 296a/ 296b | 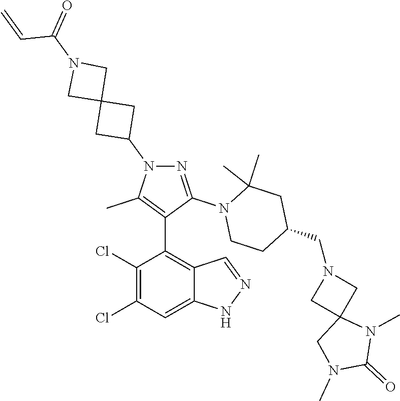 [SMILES: C=CC(=O)N1CC2(CC(n3nc(N4CC[C@@H](CN5CC6(C5)CN(C)C(=O)N6C)CC4(C)C)c(-c4c(Cl)c(Cl)cc5[nH]ncc45)c3C)C2)C1] (R)-2-((1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-5,7-dimethyl- 2,5,7-triazaspiro[3.4]octan-6- one | Using Method-13 and Intermediate A89 (Step 1), using Intermediate D6 (Step 3) and normal phase chromatography (eluent: (CH₂Cl₂/MeOH: 8/2) in CH₂Cl₂ 1 to 100%); Example 296a = 1st eluting isomer, Example 296b = 2nd eluting isomer. | Example 296b: UPLC-MS-2e: Rt = 3.82 min; MS m/z [M + H]⁺ 694.4/696.3/ 698.3, Example 296a: UPLC-MS-2e: Rt = 3.53 min; MS m/z [M + H]⁺ 694.4/696.3/ 698.3 |
| 297a/ 297b | 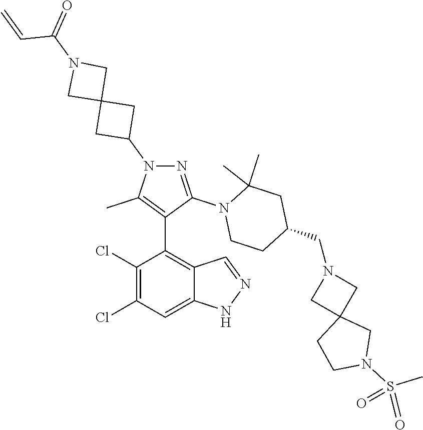 [SMILES: C=CC(=O)N1CC2(CC(n3nc(N4CC[C@@H](CN5CC6(CCN(S(C)(=O)=O)C6)C5)CC4(C)C)c(-c4c(Cl)c(Cl)cc5[nH]ncc45)c3C)C2)C1] (R)-1-(6-(4-(5,6-dichloro-1H- indazol-4-yl)-3-(2,2-dimethyl- 4-((6-(methylsulfonyl)-2,6- diazaspiro[3.4]octan-2- yl)methyl)piperidin-1-yl)-5- methyl-1H-pyrazol-1-yl)-2- azaspiro[3.3]heptan-2- yl)prop-2-en-1-one | Using Method-13 and Intermediate A90 (Step 1), using Intermediate D6 (Step 3) and normal phase chromatography (eluent: (CH₂Cl₂/MeOH: 8/2) in CH₂Cl₂ 1 to 100%) followed by reverse phase chromatography (mobile phase: 0 to 40% CH₃CN in H₂O containing 0.1% TFA); Example 297a = 1st eluting isomer, Example 297b = 2nd eluting isomer. | Example 297b: UPLC-MS-2e: Rt = 3.82 min; MS m/z [M + H]⁺ 729.3/731.3/ 733.3, Example 297a: UPLC-MS-2e: Rt = 3.53 min; MS m/z [M + H]⁺ 729.3/731.3/ 733.4 |
| 298a/ 298b | 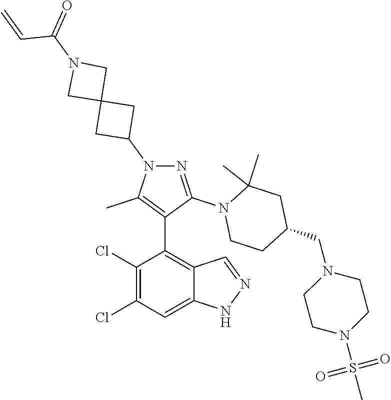 [SMILES: C=CC(=O)N1CC2(CC(n3nc(N4CC[C@@H](CN5CCN(S(C)(=O)=O)CC5)CC4(C)C)c(-c4c(Cl)c(Cl)cc5[nH]ncc45)c3C)C2)C1] (R)-1-(6-(4-(5,6-dichloro-1H- indazol-4-yl)-3-(2,2-dimethyl- 4-((4- (methylsulfonyl)piperazin-1- yl)methyl)piperidin-1-yl)-5- methyl-1H-pyrazol-1-yl)-2- azaspiro[3.3]heptan-2- yl)prop-2-en-1-one | Using Method-13 and 1- (methylsulfonyl)piperazine (Step 1), using Intermediate D6 (Step 3) and C-SFC-2 (mobile phase: CO₂/[IPA + 0.1% Et₃N 60/40); Example 298a = 1st eluting isomer, Example 298b = 2nd eluting isomer. | Example 298b: UPLC-MS-4: Rt = 0.81 min; MS m/z [M + H]⁺ 703.4/705.4/ 707.4; C-SFC-3 (mobile phase: CO₂/[IPA + 0.05% NH₃] 65/35): Rt = 2.40 min, Example 298a: C-SFC-3 (mobile phase: CO₂/[IPA + 0.05% NH₃] 65/35): Rt = 1.30 min |
| 299a/ 299b | 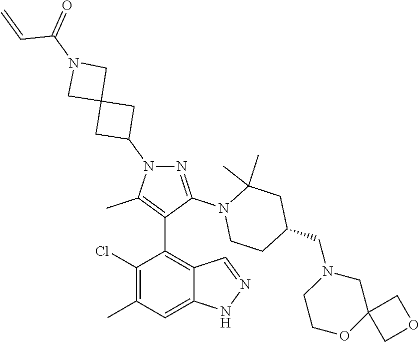 [SMILES: C=CC(=O)N1CC2(CC(n3nc(N4CC[C@@H](CN5CCOC6(COC6)C5)CC4(C)C)c(-c4c(Cl)c(C)cc5[nH]ncc45)c3C)C2)C1] (R)-1-(6-(3-(4-((2,5-dioxa-8- azaspiro[3.5]nonan-8- yl)methyl)-2,2- dimethylpiperidin-1-yl)-4-(5- chloro-6-methyl-1H-indazol- 4-yl)-5-methyl-1H-pyrazol-1- yl)-2-azaspiro[3.3]heptan-2- yl)prop-2-en-1-one | Using Method-13ef and 2,5-dioxa-8- azaspiro[3.5]nonane (Step 1), using Intermediate D1 (Step 3) and C-SFC-35 (mobile phase: CO₂/[IPA + 0.5% Et₃N 70/30); Example 299a = 1st eluting isomer, Example 299b = 2nd eluting isomer. | Example 299b: UPLC-MS-4: Rt = 0.88 min; MS m/z [M + H]⁺ 648.7/650.6; C-SFC-3 (mobile phase: CO₂/[IPA + 0.05% NH₃] 70/30): Rt = 2.40 min, Example 299a: UPLC-MS-4: Rt = 0.88 min; MS m/z [M + H]⁺ 648.4/650.4; C-SFC-3 (mobile phase: CO₂/[IPA + 0.05% NH₃] 70/30): Rt = 1.80 min. |
| 300a/ 300b | 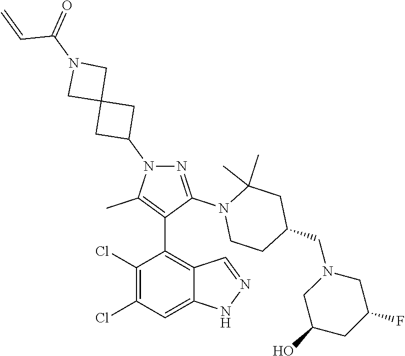 [SMILES: C=CC(=O)N1CC2(CC(n3nc(N4CC[C@@H](CN5C[C@H](O)C[C@@H](F)C5)CC4(C)C)c(-c4c(Cl)c(Cl)cc5[nH]ncc45)c3C)C2)C1] 1-(6-(4-(5,6-dichloro-1H- indazol-4-yl)-3-((R)-4- (((3R*,5R*)-3-fluoro-5- hydroxypiperidin-1- yl)methyl)-2,2- dimethylpiperidin-1-yl)-5- methyl-1H-pyrazol-1-yl)-2- azaspiro[3.3]heptan-2- yl)prop-2-en-1-one | Using Method-13e and (3R*,5R*)-5- fluoropiperidin-3-ol hydrochloride (Step 1), using Intermediate D6 (Step 3) and C-SFC-4 (mobile phase: CO₂/[IPA + 0.5% Et₃N 72/28); Example 300a = 1st eluting isomer, Example 300b = 2nd eluting isomer. | Example 300b: UPLC-MS-4: Rt = 0.81 min; MS m/z [M + H]⁺ 658.6/660.5/ 662.5; C-SFC-3 (mobile phase: CO₂/[IPA + 0.05% NH₃] 72/28): Rt = 2.70 min, Example 300a: UPLC-MS-4: Rt = 0.76 min; MS m/z [M + H]⁺ 658.4/660.4/ 662.4; C-SFC-3 (mobile phase: CO₂/[IPA + 0.05% NH₃] 72/28): Rt = 1.90 min |
| 301a/ 301b | 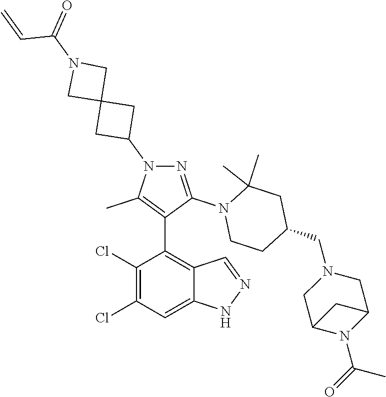 [SMILES: C=CC(=O)N1CC2(CC(n3nc(N4CC[C@@H](CN5CC6CC(C5)N6C(C)=O)CC4(C)C)c(-c4c(Cl)c(Cl)cc5[nH]ncc45)c3C)C2)C1] 1-(6-(3-((4R)-4-((6-acetyl- 3,6- diazabicyclo[3.1.1]heptan-3- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13e and 1- (3,6- diazabicyclo[3.1.1]heptan- 6-yl)ethan-1-one [CAS: 1474024-25-3] (Step 1) using Intermediate D6 (Step 3) and RP-HPLC-2 (mobile phase: 5 to 45% CH₃CN in H₂O containing 0.1% TFA); Example 301a = 1st eluting isomer, Example 301b = 2nd eluting isomer. | Example 301b: UPLC-MS-4: Rt = 0.80 min; MS m/z [M + H]⁺ 679.6/681.6/ 683.6; C-SFC-8 (mobile phase: CO₂/[IPA + 0.05% NH₃] 60/40): Rt = 3.75 min, Example 301a: UPLC-MS-4: Rt = 0.75 min; MS m/z [M + H]⁺ 679.5/681.4/ 683.4; C-SFC-8 (mobile phase: CO₂/[IPA + 0.05% NH₃] 60/40): Rt = 2.65 min. |
| 302a/ 302b | 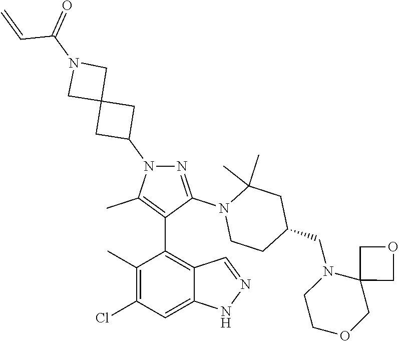 [SMILES: C=CC(=O)N1CC2(CC(n3nc(N4CC[C@@H](CN5CCOCC56COC6)CC4(C)C)c(-c4c(C)c(Cl)cc5[nH]ncc45)c3C)C2)C1] (R)-1-(6-(3-(4-((2,8-dioxa-5- azaspiro[3.5]nonan-5- yl)methyl)-2,2- dimethylpiperidin-1-yl)-4-(6- chloro-5-methyl-1H-indazol- 4-yl)-5-methyl-1H-pyrazol-1- yl)-2-azaspiro[3.3]heptan-2- yl)prop-2-en-1-one | Using Method-13ce and 2,8-dioxa-5- azaspiro[3.5]nonane (Step 1), using Intermediate D10 (Step 3) and RP-HPLC-2 (mobile phase: 5 to 45% CH₃CN in H₂O containing 0.1% TFA); Example 302a = 1st eluting isomer, Example 302b = 2nd eluting isomer. | Example 302b: UPLC-MS-4: Rt = 1.07 min; MS m/z [M + H]⁺ 648.5/650.5; C-SFC-3 (mobile phase: CO₂/[IPA + 0.05% NH₃] 75/25): Rt 3.55 min, Example 302a: UPLC-MS-4: Rt = 1.05 min; MS m/z [M + H]⁺ 648.5/650.5; C-SFC-3 (mobile phase: CO₂/[IPA + 0.05% NH₃] 75/25): Rt = 3.13 min. |
| 303a/ 303b | 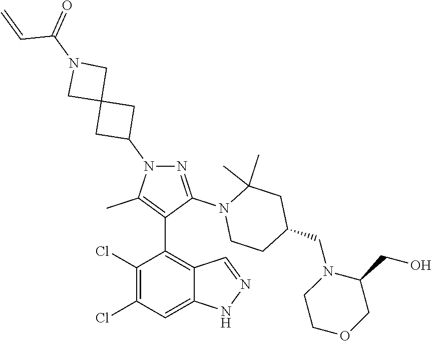 [SMILES: C=CC(=O)N1CC2(CC(n3nc(N4CC[C@@H](CN5CCOC[C@@H]5CO)CC4(C)C)c(-c4c(Cl)c(Cl)cc5[nH]ncc45)c3C)C2)C1] 1-(6-(4-(5,6-dichloro-1H- indazol-4-yl)-3-((R)-4-(((S)-3- (hydroxymethyl)morpholino) methyl)-2,2- dimethylpiperidin-1-yl)-5- methyl-1H-pyrazol-1-yl)-2- azaspiro[3.3]heptan-2- yl)prop-2-en-1-one | Using Method-13 and (S)- 3- hydroxymethylmorpholine (Step 1), using Intermediate D6 (Step 3) and C-SFC-4 (mobile phase: CO₂/[IPA + 0.1% Et₃N] 70/30); Example 303a = 1st eluting isomer, Example 303b = 2nd eluting isomer. | Example 303b: C- SFC-3 (mobile phase: CO₂/[IPA + 0.025% NH₃] 70/30): Rt = 2.91 min; UPLC-MS-2e: Rt = 3.70 min; MS m/z [M + H]⁺ 656.3/658.3/ 660.3, Example 303a: C-SFC-3 (mobile phase: CO₂/[IPA + 0.025% NH₃] 70/30): Rt = 1.71. min. |
| 304a/ 304b | 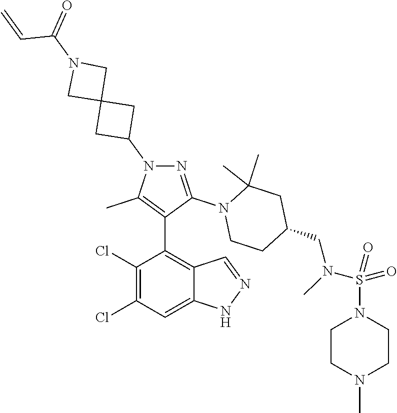 [SMILES: C=CC(=O)N1CC2(CC(n3nc(N4CC[C@@H](CN(C)S(=O)(=O)N5CCN(C)CC5)CC4(C)C)c(-c4c(Cl)c(Cl)cc5[nH]ncc45)c3C)C2)C1] (R)-N-((1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-N,4- dimethylpiperazine-1- sulfonamide | Using Method-13 and Intermediate C117 (Step 2) and using Intermediate D6 (Step 3) and RP-HPLC-2 (mobile phase: 10 to 40% CH₃CN in H₂O containing 0.1% TFA); Example 304a = 1st eluting isomer, Example 304b = 2nd eluting isomer. | Example 304b: UPLC-MS-2e: Rt = 4.22 min; MS m/z [M + H]⁺ 732.3/734.3/ 736.3, Example 304a: UPLC-MS-2e: Rt = 3.98 min; MS m/z [M + H]⁺ 732.3/734.3/ 736.3. min. |
| 305a/ 305b | 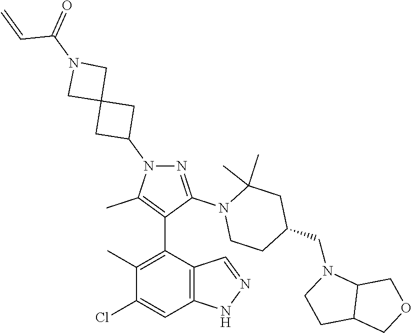 [SMILES: C=CC(=O)N1CC2(CC(n3nc(N4CC[C@@H](CN5CCC6COCC65)CC4(C)C)c(-c4c(C)c(Cl)cc5[nH]ncc45)c3C)C2)C1] 1-(6-(4-(6-chloro-5-methyl- 1H-indazol-4-yl)-3-((R)-4- (((3aR*,6aR*)-hexahydro- 1H-furo[3,4-b]pyrrol-1- yl)methyl)-2,2- dimethylpiperidin-1-yl)-5- methyl-1H-pyrazol-1-yl)-2- azaspiro[3.3]heptan-2- yl)prop-2-en-1-one | Using Method-13c and Intermediate C119a (Step 2), using Intermediate D10 (Step 3) and normal phase chromatography (eluent: MeOH in CH₂Cl₂ from 0 to 10%); Example 305a = 1st eluting isomer, Example 305b = 2nd eluting isomer. | Example 305b: UPLC-MS-2e: Rt = 3.76 min; MS m/z [M + H]⁺ 632.4/634.4, Example 305a: UPLC-MS-2e: Rt = 3.48 min; MS m/z [M + H]⁺ 632.4/634.4. |
| 306a/ 306b | 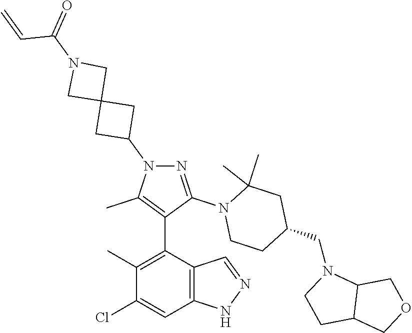 [SMILES: C=CC(=O)N1CC2(CC(n3nc(N4CC[C@@H](CN5CCC6COCC65)CC4(C)C)c(-c4c(C)c(Cl)cc5[nH]ncc45)c3C)C2)C1] 1-(6-(4-(6-chloro-5-methyl- 1H-indazol-4-yl)-3-((R)-4- (((3aR*,6aR*)-hexahydro- 1H-furo[3,4-b]pyrrol-1- yl)methyl)-2,2- dimethylpiperidin-1-yl)-5- methyl-1H-pyrazol-1-yl)-2- azaspiro[3.3]heptan-2- yl)prop-2-en-1-one | Using Method-13c and Intermediate C119b (Step 2), using Intermediate D10 (Step 3) and normal phase chromatography (eluent: MeOH in CH₂Cl₂ from 0 to 10%); Example 306a = 1st eluting isomer, Example 306b = 2nd eluting isomer. | Example 306b: UPLC-MS-4: Rt = 0.78 min; MS m/z [M + H]⁺ 632.4/634.4, Example 306a: UPLC-MS-4: Rt = 0.76 min; MS m/z [M + H]⁺ 632.4/634.4. |
| 307a/ 307b | 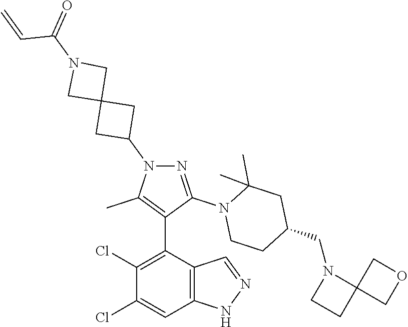 [SMILES: C=CC(=O)N1CC2(CC(n3nc(N4CC[C@@H](CN5CCC56COC6)CC4(C)C)c(-c4c(Cl)c(Cl)cc5[nH]ncc45)c3C)C2)C1] (R)-1-(6-(3-(4-((6-oxa-1- azaspiro[3.3]heptan-1- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13e and 6- oxa-1- azaspiro[3.3]heptane oxalate salt (Step 1), using Intermediate D6 (Step 3) and RP-HPLC-2 (mobile phase: 5 to 30% CH₃CN in H₂O containing 0.1% TFA); Example 307a = 1st eluting isomer, Example 307b = 2nd eluting isomer | Example 307b: UPLC-MS-4: Rt = 0.82 min; MS m/z [M + H]⁺ 638.5/640.5/ 642.5; C-SFC-3 (mobile phase: CO₂/[IPA + 0.05% NH₃] 70/30): Rt = 2.27 min; Example 307a: UPLC-MS-4: Rt = 0.78 min; MS m/z [M + H]⁺ 638.5/640.5/ 642.5; C-SFC-3 (mobile phase: CO₂/[IPA + 0.05% NH₃] 70/30): Rt = 1.44 min |
| 308a/ 308b | 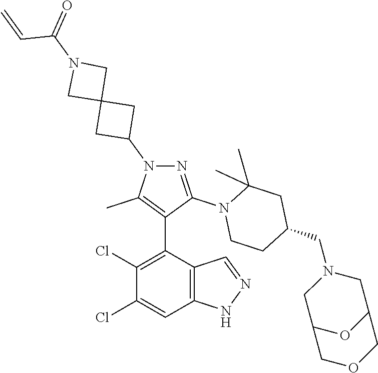 [SMILES: C=CC(=O)N1CC2(CC(n3nc(N4CC[C@@H](CN5CC6COCC(C5)O6)CC4(C)C)c(-c4c(Cl)c(Cl)cc5[nH]ncc45)c3C)C2)C1] 1-(6-(3-((4R)-4-((3,9-dioxa-7- azabicyclo[3.3.1]nonan-7- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13d and 3,9- dioxa-7- azabicyclo[3.3.1]nonane hydrochloride [1803587- 96-3] (Step 1), using Intermediate D6 (Step 3) and C-SFC-25: (mobile phase: CO₂/[IPA + 0.05% Et₃N] 70/30); Example 308a = 1st eluting atropisomer Example 308b = 2nd eluting atropisomer | Example 308b: ¹H NMR (400 MHz, DMSO-d₆) δ 13.2 (s, 1H), 7.77 (s, 1H), 7.65 (s, 1H), 6.30 (m, 1H), 6.09 (m, 1H), 5.67 (m, 1H), 4.76 (m, 1H), 4.33 (s, 1H), 4.28 (s, 1H), 4.05 (s, 1H), 4.00 (s, 1H), 3.70-3.78 (m, 2H), 3.60-3.69 (m, 2H), 3.57 (m, 2H), 3.17 (m, 1H), 2.62- 2.88 (m, 7H), 2.28 (m, 1H), 2.21 (m, 1H), 2.01 (s, 3H), 1.72- 1.89 (m, 3H), 1.63 (m, 1H), 1.34 (m, 1H), 1.13 (s, 3H), 0.71 (m, 3H), 0.67 (m, 1H), 0.62 (m, 1H); UPLC- MS-4: Rt = 0.80 min; MS m/z [M + H]⁺ 668.4/ 670.3/672.3; C- SFC-3 (mobile phase: CO₂/[IPA + 0.05% NH₃] 70/30): Rt = 2.31 min, Example 308a: C-SFC-3 (mobile phase: CO₂/[IPA + 0.05% NH₃] 70/30): Rt = 1.50 min. |
| 309a/ 309b | 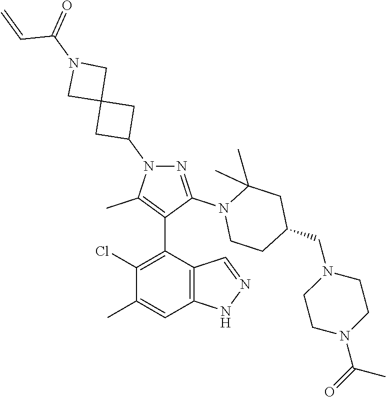 [SMILES: C=CC(=O)N1CC2(CC(n3nc(N4CC[C@@H](CN5CCN(C(C)=O)CC5)CC4(C)C)c(-c4c(Cl)c(C)cc5[nH]ncc45)c3C)C2)C1] (R)-1-(6-(3-(4-((4- acetylpiperazin-1-yl)methyl)- 2,2-dimethylpiperidin-1-yl)-4- (5-chloro-6-methyl-1H- indazol-4-yl)-5-methyl-1H- pyrazol-1-yl)-2- azaspiro[3.3]heptan-2- yl)prop-2-en-1-one | Using Method-13b and 1- (piperazin-1-yl)ethan-1-one (CAS [13889-98-0]) (Step 1), using Intermediate D1 (Step 3) and RP-HPLC-2: (mobile phase: [H₂O + 0.1% TFA]/CH₃CN 95:5 to 0:100 in 17 min); Example 309a = 1st eluting atropisomer Example 309b = 2nd eluting atropisomer | Example 309b: UPLC-MS-4: Rt = 0.77 min; MS m/z [M + H]⁺ 647.4/649.4; RP-HPLC-2: (mobile phase: [H₂O + 0.1% TFA]/CH₃CN 95:5 to 0:100 in 17 min): Rt = 8.47 min, Example 309a: RP-HPLC-2: (mobile phase: [H₂O + 0.1% TFA]/CH₃CN 95:5 to 0:100 in 17 min): Rt = 8.28 min. |
| 310a/ 310b | 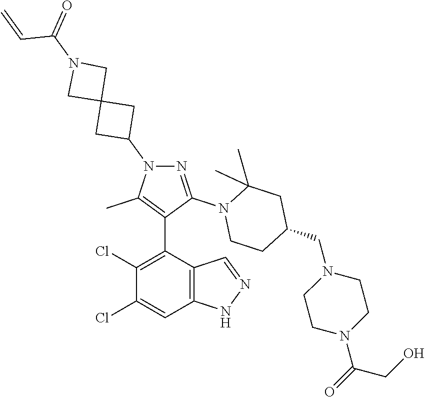 [SMILES: C=CC(=O)N1CC2(CC(n3nc(N4CC[C@@H](CN5CCN(C(=O)CO)CC5)CC4(C)C)c(-c4c(Cl)c(Cl)cc5[nH]ncc45)c3C)C2)C1] (R)-1-(6-(4-(5,6-dichloro-1H- indazol-4-yl)-3-(4-((4-(2- hydroxyacetyl)piperazin-1- yl)methyl)-2,2- dimethylpiperidin-1-yl)-5- methyl-1H-pyrazol-1-yl)-2- azaspiro[3.3]heptan-2- yl)prop-2-en-1-one | Using Method-13d and 2- hydroxy-1-(piperazin-1- yl)ethan-1-one (CAS [117701-75-4]) (Step 1), using Intermediate D6 (Step 3) and RP-HPLC-2: (mobile phase: [H₂O + 0.1% TFA]/CH₃CN 95:5 to 0:100 in 17 min); Example 310a = 1st eluting atropisomer Example 310b = 2nd eluting atropisomer | Example 310b: UPLC-MS-4: Rt = 0.76 min; MS m/z [M + H]⁺ 683.2/685.2/ 687.1; RP-HPLC-2 (mobile phase: [H₂O + 0.1% TFA]/CH₃CN 95:5 to 0:100 in 17 min): Rt = 7.30 min, Example 310a: RP- HPLC-2 (mobile phase: [H₂O + 0.1% TFA]/CH₃CN 95:5 to 0:100 in 17 min): Rt = 6.45 min. |
| 311a/ 311b | 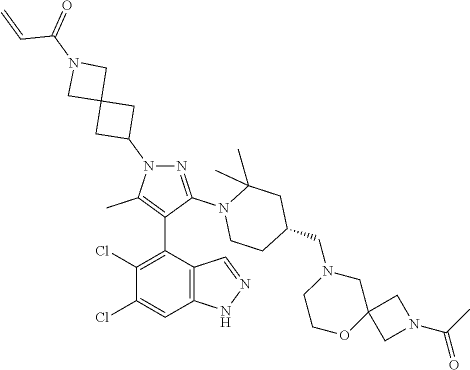 [SMILES: C=CC(=O)N1CC2(CC(n3nc(N4CC[C@@H](CN5CCOC6(C5)CN(C(C)=O)C6)CC4(C)C)c(-c4c(Cl)c(Cl)cc5[nH]ncc45)c3C)C2)C1] (R)-1-(6-(3-(4-((2-acetyl-5- oxa-2,8- diazaspiro[3.5]nonan-8- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13d and Intermediate A25 (Step 1), using Intermediate D6 (Step 3) and RP-HPLC-2: (mobile phase: [H₂O + 0.1% TFA]/CH₃CN 95:5 to 31:69 in 11.5 min); Example 311a = 1st eluting atropisomer Example 311b = 2nd eluting atropisomer | Example 311b: UPLC-MS-4: Rt = 0.92 min; MS m/z [M + H]⁺ 709.2/711.3/ 713.3; RP-HPLC-2 (mobile phase: [H₂O + 0.1% TFA]/CH₃CN 95:5 to 31:69 in 11.5 min): Rt = 7.25 min, Example 311a: RP- HPLC-2 (mobile phase: [H₂O + 0.1% TFA]/CH₃CN 95:5 to 31:69 in 11.5 min): Rt = 6.48 min. |
| 312a/ 312b | 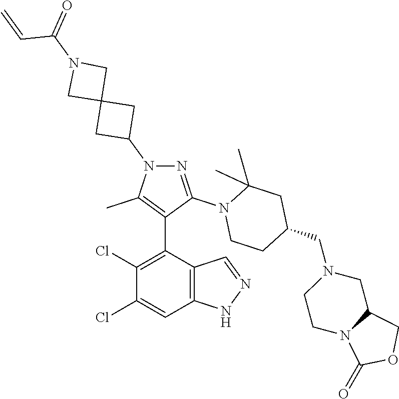 (S)-7-(((R)-1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)hexahydro-3H- oxazolo[3,4-a]pyrazin-3-one | Using Method-13be and (S)-hexahydro-3H- oxazolo[3,4-a]pyrazin-3- one hydrochloride (CAS [958635-15-9]) (Step 1), using Intermediate D6 (Step 3) and reverse phase chromatography (mobile phase: [H₂O + 0.1% TFA]/CH₃CN 90:10 to 68:32 to 50:50 in 15 min); Example 312a = 1st eluting atropisomer Example 312b = 2nd eluting atropisomer | Example 312b: UPLC-MS-4: Rt = 0.87 min; MS m/z [M + H]⁺ 681.3/683.3/ 685.3, Example 312a: UPLC-MS-4: Rt = 0.83 min; MS m/z [M + H]⁺ 681.3/683.3/ 685.3. |
| 313a/ 313b | 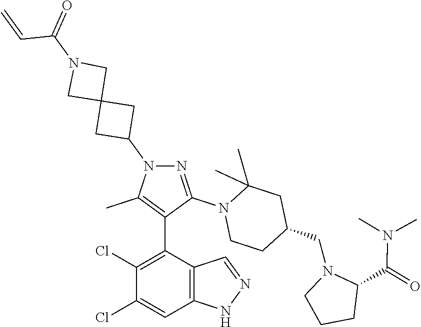 [SMILES: C=CC(=O)N1CC2(CC(n3nc(N4CC[C@@H](CN5CCC[C@H]5C(=O)N(C)C)CC4(C)C)c(-c4c(Cl)c(Cl)cc5[nH]ncc45)c3C)C2)C1] (S)-1-(((R)-1-(1-(2-acryloyl-2- azaspiro[3.3]heptan-6-yl)-4- (5,6-dichloro-1H-indazol-4- yl)-5-methyl-1H-pyrazol-3- yl)-2,2-dimethylpiperidin-4- yl)methyl)-N,N- dimethylpyrrolidine-2- carboxamide | Using Method-13be and (S)-N,N- dimethylpyrrolidine-2- carboxamide (CAS [29802- 22-0]) (Step 1), using Intermediate D6 (Step 3) and reverse phase chromatography (mobile phase: [H₂O + 0.1% TFA]/CH₃CN 90:10 to 68:32 to 60:40 in 12 min); Example 313a = 1st eluting atropisomer Example 313b = 2nd eluting atropisomer | Example 313b: UPLC-MS-4: Rt = 0.78 min; MS m/z [M + H]⁺ 681.7/683.6/ 685.6, Example 313a: UPLC-MS-4: Rt = 0.72 min; MS m/z [M + H]⁺ 681.7/683.6/ 685.6. |
| 314a/ 314b | 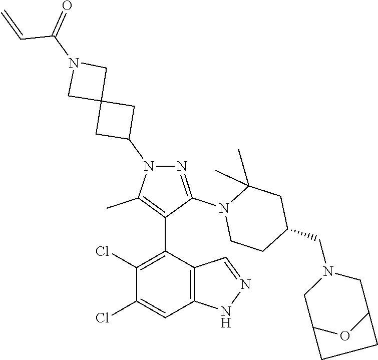 [SMILES: C=CC(=O)N1CC2(CC(n3nc(N4CC[C@@H](CN5CC6CCC(C5)O6)CC4(C)C)c(-c4c(Cl)c(Cl)cc5[nH]ncc45)c3C)C2)C1] 1-(6-(3-((4R)-4-((8-oxa-3- azabicyclo[3.2.1]octan-3- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13be and 8- oxa-3- azabicyclo[3.2.1]octane hydrochloride (CAS [54745-74-3]) (Step 1), using Intermediate D6 (Step 3) and C-SFC-4 (mobile phase: CO₂/[IPA + 0.1% Et₃N] 70/30); Example 314a = 1st eluting atropisomer Example 314b = 2nd eluting atropisomer | Example 314b: UPLC-MS-4: Rt = 0.86 min; MS m/z [M + H]⁺ 652.6/654.6/ 656.6; C-SFC-3 (mobile phase: CO₂/[IPA + 0.05% NH₃] 70/30): Rt = 2.17 min, Example 314a: C- SFC-3 (mobile phase: CO₂/[IPA + 0.05% NH₃] 70/30): Rt = 1.45 min. |
| 315a/ 315b | 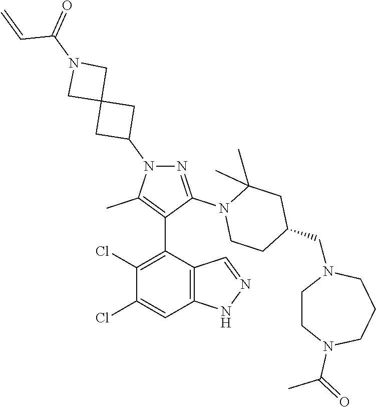 [SMILES: C=CC(=O)N1CC2(CC(n3nc(N4CC[C@@H](CN5CCCN(C(C)=O)CC5)CC4(C)C)c(-c4c(Cl)c(Cl)cc5[nH]ncc45)c3C)C2)C1] (R)-1-(6-(3-(4-((4-acetyl-1,4- diazepan-1-yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13be and 1- (1,4-diazepan-1-yl)ethan- 1-one (CAS [61903-11-5]) (Step 1), using Intermediate D6 (Step 3) and reverse phase chromatography (mobile phase: [H₂O + 0.1% TFA]/CH₃CN 90:10 to 55:45 in 14 min); Example 315a = 1st eluting atropisomer Example 315b = 2nd eluting atropisomer | Example 315b: UPLC-MS-4: Rt = 0.75 min; MS m/z [M + H]⁺ 681.6/683.6/ 685.6, Example 315a: UPLC-MS-4: Rt = 0.71 min; MS m/z [M + H]⁺ 681.6/683.6/ 685.6. |
| 316a/ 316b | 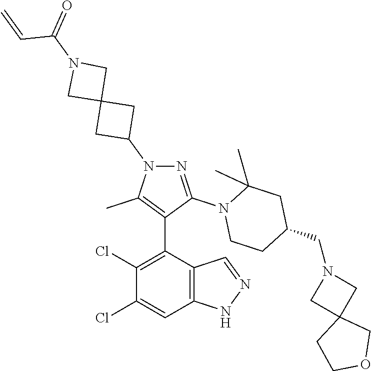 [SMILES: C=CC(=O)N1CC2(CC(n3nc(N4CC[C@@H](CN5CC6(CCOC6)C5)CC4(C)C)c(-c4c(Cl)c(Cl)cc5[nH]ncc45)c3C)C2)C1] (R)-1-(6-(3-(4-((6-oxa-2- azaspiro[3.4]octan-2- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13be and 6- oxa-2-azaspiro[3.4]octane hemi-oxalate (CAS [1523571-05-2]) (Step 1), using Intermediate D6 (Step 3) and reverse phase chromatography (mobile phase: [H₂O + 0.1% TFA]/CH₃CN 90:10 to 40:60 in 11 min); Example 316a = 1st eluting atropisomer Example 316b = 2nd eluting atropisomer | Example 316b: UPLC-MS-4: Rt = 0.80 min; MS m/z [M + H]⁺ 652.3/654.3/ 656.3, Example 316a: UPLC-MS-4: Rt = 0.75 min; MS m/z [M + H]⁺ 652.3/654.3/ 656.3. |
| 317a/ 317b | 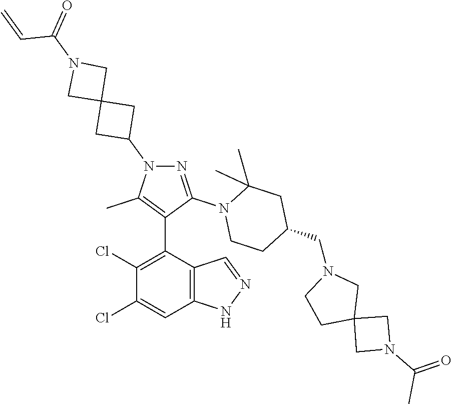 [SMILES: C=CC(=O)N1CC2(CC(n3nc(N4CC[C@@H](CN5CCC6(C5)CN(C(C)=O)C6)CC4(C)C)c(-c4c(Cl)c(Cl)cc5[nH]ncc45)c3C)C2)C1] (R)-1-(6-(3-(4-((2-acetyl-2,6- diazaspiro[3.4]octan-6- yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13be and Intermediate A26, using Intermediate D6 (Step 3) and reverse phase chromatography (mobile phase: [H₂O + 0.1% TFA]/CH₃CN 100:0 to 70:30 to 45:55 in 14.5 min); Example 317a = 1st eluting atropisomer Example 317b = 2nd eluting atropisomer | Example 317b: UPLC-MS-4: Rt = 0.75 min; MS m/z [M + H]⁺ 693.3/695.4/ 697.4, Example 317a: UPLC-MS-4: Rt = 0.70 min; MS m/z [M + H]⁺ 693.3/695.4/ 697.4. |
| 318a/ 318b | 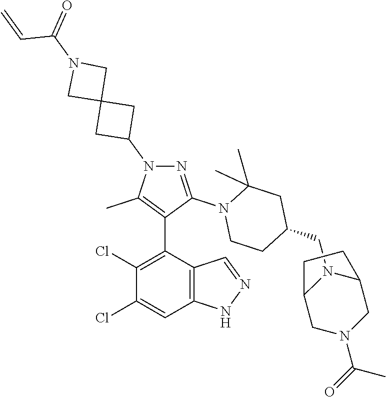 [SMILES: C=CC(=O)N1CC2(CC(n3nc(N4CC[C@@H](CN5C6CCC5CN(C(C)=O)C6)CC4(C)C)c(-c4c(Cl)c(Cl)cc5[nH]ncc45)c3C)C2)C1] 1-(6-(3-((4R)-4-((3-acetyl- 3,8-diazabicyclo[3.2.1]octan- 8-yl)methyl)-2,2- dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13be and Intermediate A12, using Intermediate D6 (Step 3) and reverse phase chromatography (mobile phase: [H₂O + 0.1% TFA]/CH₃CN 100:0 to 77:23 to 67:33 to 55:45 in 22 min); Example 318a = 1st eluting atropisomer Example 318b = 2nd eluting atropisomer | Example 318b: UPLC-MS-4: Rt = 0.77 min; MS m/z [M + H]⁺ 693.5/695.5/ 697.5, Example 318a: UPLC-MS-4: Rt = 0.73 min; MS m/z [M + H]⁺ 693.5/695.5/ 697.5. |
| 319a/ 319b | 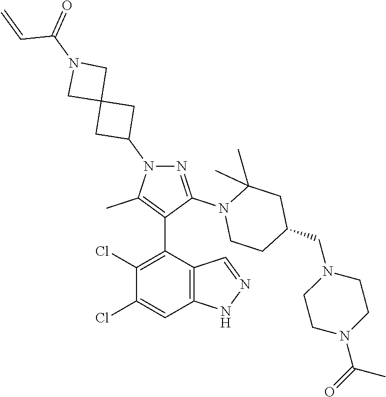 [SMILES: C=CC(=O)N1CC2(CC(n3nc(N4CC[C@@H](CN5CCN(C(C)=O)CC5)CC4(C)C)c(-c4c(Cl)c(Cl)cc5[nH]ncc45)c3C)C2)C1] (R)-1-(6-(3-(4-((4- acetylpiperazin-1-yl)methyl)- 2,2-dimethylpiperidin-1-yl)-4- (5,6-dichloro-1H-indazol-4- yl)-5-methyl-1H-pyrazol-1- yl)-2-azaspiro[3.3]heptan-2- yl)prop-2-en-1-one | Using Method-13b and 1- (piperazin-1-yl)ethan-1-one (CAS [13889-98-0]) (Step 1), using Intermediate D6 (Step 3) and RP-HPLC-2: (mobile phase: [H₂O + 0.1% TFA]/CH₃CN 95:5 to 0:100 in 17 min); Example 319a = 1st eluting atropisomer Example 319b = 2nd eluting atropisomer | Example 319b: ¹H NMR (400 MHz, DMSO-d₆) δ 13.2 (br s, 1H), 7.78 (s, 1H), 7.64 (s, 1H), 6.30 (m, 1H), 6.10 (m, 1H), 5.67 (m, 1H), 4.75 (m, 1H), 4.33 (s, 1H), 4.28 (s, 1H), 4.05 (s, 1H), 4.00 (s, 1H), 3.30- 3.40 (m, 4H), 3.17 (m, 1H), 2.79 (m, 1H), 2.64-2.76 (m, 4H), 2.12-2.32 (m, 4H), 2.01 (s, 3H), 1.95 (s, 3H), 1.94 (m, 2H), 1.78 (m, 1H), 1.54 (m, 1H), 1.36 (m, 1H), 1.13 (s, 3H), 0.72 (m, 3H), 0.72 (m, 1H), 0.61 (m, 1H); UPLC- MS-4: Rt = 0.79 min; MS m/z [M + H]⁺ 667.3/ 669.4/671.4; RP- HPLC-2 (mobile phase: [H₂O + 0.1% TFA]/CH₃CN 95:5 to 0:100 in 17 min): Rt = 7.41 min, Example 319a: RP-HPLC-2 (mobile phase: [H₂O + 0.1% TFA]/CH₃CN 95:5 to 0:100 in 17 min): Rt = 6.71 min. |
| 320a/ 320b | 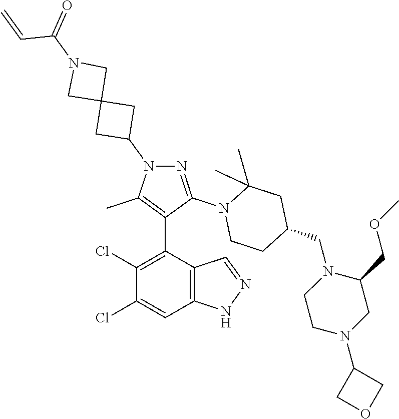 [SMILES: C=CC(=O)N1CC2(CC(n3nc(N4CC[C@@H](CN5CCN(C6COC6)C[C@@H]5COC)CC4(C)C)c(-c4c(Cl)c(Cl)cc5[nH]ncc45)c3C)C2)C1] 1-(6-(4-(5,6-dichloro-1H- indazol-4-yl)-3-((R)-4-(((R)-2- (methoxymethyl)-4-(oxetan- 3-yl)piperazin-1-yl)methyl)- 2,2-dimethylpiperidin-1-yl)-5- methyl-1H-pyrazol-1-yl)-2- azaspiro[3.3]heptan-2- yl)prop-2-en-1-one | Using Method-13d and Intermediate A6 (Step 1), using Intermediate D6 (Step 3) and RP-HPLC-2: (mobile phase: [H₂O + 0.1% TFA]/CH₃CN 95:5 to 0:100 in 17 min); Example 320a = 1st eluting atropisomer Example 320b = 2nd eluting atropisomer | Example 320b: ¹H NMR (600 MHz, DMSO-d₆) δ 13.22 (br. s, 1H), 7.78 (s, 1H), 7.63 (s, 1H), 6.30 (m, 1H), 6.09 (m, 1H), 5.67 (m, 1H), 4.75 (m, 1H), 4.49 (m, 2H), 4.36 (m, 2H), 4.33 (s, 1H), 4.28 (s, 1H), 4.05 (s, 1H), 4.00 (s, 1H), 3.38 (m, 1H), 3.31- 3.29 (m, 1H), 3.24- 3.14 (m, 2H), 3.18 (s, 3H), 2.80 (m, 1H), 2.76-2.63 (m, 5H), 2.41-2.36 (m, 2H), 2.33-2.26 (m, 2H), 2.17 (m, 1H), 2.08- 1.96 (m, 1H), 2.00 (s, 3H), 1.95-1.86 (m, 2H), 1.70 (m, 1H), 1.48 (m, 1H), 1.39 (m, 1H), 1.10 (s, 1.5H), 1.09 (s, 1.5H), 0.71 (m, 1.5H), 0.70 (s, 1.5H), 0.68 (m, 1H), 0.55 (m, 1H); UPLC- MS-4: Rt = 0.82 min; MS m/z [M + H]⁺ 725.3/ 727.3/729.3, Example 320a: UPLC-MS-4: Rt = 0.79 min; MS m/z [M + H]⁺ 725.3/727.3/ 729.3. |

### Method-A1 for the Synthesis of Intermediate A1: 8-(Oxetan-3-yl)-5,8-diazaspiro[3.5]nonane

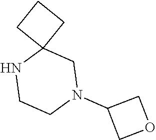 [SMILES: C1CC2(C1)CN(C1COC1)CCN2]

### Step 1: Tert-butyl 8-(oxetan-3-yl)-5,8-diazaspiro[3.5]nonane-5-carboxylate

In an ace tube, to a colorless solution of tert-butyl 5,8-diazaspiro[3.5]nonane-5-carboxylate (4 g, 17.67 mmol) and oxetan-3-one (1.70 mL, 26.5 mmol) in dichloethane (70 mL) was added sodium triacetoxyborohydride (5.99 g, 28.3 mmol). The reaction mixture was stirred at RT for 3.5 days. The reaction mixture was poured into an aq. sat. NaHCO₃ solution and extracted with CH₂Cl₂ (×2). The combined organic layers were dried (phase separator) and concentrated to give the title compound as a brown oil (5.19 g). MS m/z [M+H]⁺ 283.3; MS 1.

### Step 2: 8-(Oxetan-3-yl)-5,8-diazaspiro[3.5]nonane

Tert-butyl 8-(oxetan-3-yl)-5,8-diazaspiro[3.5]nonane-5-carboxylate (Step 1, 2.02 g, 6.87 mmol) was dissolved in CH₂Cl₂ (25 mL). TFA was added (5.29 mL, 68.7 mmol) and the solution was stirred at RT for 2.5 h. The reaction mixture was concentrated and to the crude residue dissolved in MeOH (50 mL) was added MP-carbonate (loading: 3.03 mmol/g, 10 eq, 68.7 mmol, 22.7 g). The mixture was shaked at RT for 1 h and filtered, washed with MeOH and the filtrate was concentrated under reduced pressure to give the title compound as a TFA salt as a light brown foam (1.97 g) which was used without further purification in the next step. MS m/z [M+H]⁺ 183.2; MS 1.

Method-A1a: Similar to Method-A1 except that the final compound was further purified on basic alumina (eluent: MeOH in CH₂Cl₂) to obtain the free base.

The following intermediates A2 to A6 were prepared using analogous methods from commercially available reagents.

| | | | Characterizing |
|---|---|---|---|
| Intermediate | Structure | Precursor | data |
| A2 | 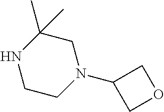 | From tert-butyl 2,2- dimethylpiperazine-1- carboxylate | MS-1; MS m/z [M + H]⁺ 171.1 |
| | 3,3-dimethyl-1-(oxetan-3- | | |
| | yl)piperazine | | |
| A3 | 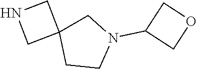 [SMILES: C1OCC1N1CCC2(CNC2)C1] | Using Method-A1a from tert-butyl 2,6- diazaspiro[3,4]octane- carboxylate | UPLC-MS-4: Rt = 0.23 min; MS m/z [M + H]⁺ 169.3 |
| | 6-(oxetan-3-yl)-2,6- | | |
| | diazaspiro[3.4]octane | | |
| A4 | 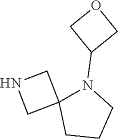 [SMILES: C1CN(C2COC2)C2(C1)CNC2] | Using Method-A1a from tert-butyl 2,5- diazaspiro[3.4]octane-2- carboxylate | UPLC-MS-4: Rt = 0.23 min; MS m/z [M + H]⁺ 169.2 |
| | 5-(oxetan-3-yl)-2,5- | | |
| | diazaspiro[3.4]octane | | |
| A5 | 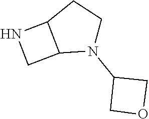 [SMILES: C1CN(C2COC2)C2CNC12] | Using Method-A1a from rac 2,6-diaza- bicyclo[3.2.0]heptane-6- carboxylic acid tert-butyl ester | UPLC-MS-4: Rt = 0.13 min; MS m/z [M + H]⁺ 155.2 |
| | Rac-(1S*,5S*)-2-(oxetan- | | |
| | 3-yl)-2,6- | | |
| | diazabicyclo[3.2.0]heptane | | |
| A6 | 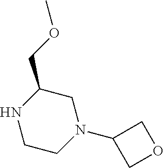 | Using Method-A1a from tert-butyl (R)-2- (methoxymethyl)piperazine- 1-carboxylate (CAS [1023301-73-6]) | MS-1: MS m/z [M + H]⁺ 187.2 |
| | (R)-3-(methoxymethyl)-1- | | |
| | (oxetan-3-yl)piperazine | | |

### Intermediate A7: (R)-(4-(Oxetan-3-yl)piperazin-2-yl)methanol

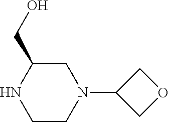 [SMILES: OC[C@H]1CN(C2COC2)CCN1]

### Step 1: (R)-(1-Benzyl-4-(oxetan-3-yl)piperazin-2-yl)methanol

NaBH(OAc)₃ (6.16 g, 29.1 mmol) was added under Ar atmosphere at 0° C. to a solution of (R)-(1-benzylpiperazin-2-yl)methanol (2.00 g, 9.70 mmol) and oxetan-3-one (0.93 mL, 14.5 mmol) in dichloroethane (40 mL). The reaction mixture was stirred at RT for 2 h. Then, the RM was poured into an aq. sat. NaHCO₃ solution and was extracted with CH₂Cl₂ (×3). The combined organic layer was dried (phase separator) and evaporated. The crude residue was purified by normal phase chromatography on basic alumina (eluent: MeOH in CH₂Cl₂ from 0 to 3%) to give the title product. UPLC-MS-4: Rt=0.12 min; MS m/z [M+H]⁺ 263.3.

### Step 2: (R)-(4-(Oxetan-3-yl)piperazin-2-yl)methanol

A solution of (R)-(1-benzyl-4-(oxetan-3-yl)piperazin-2-yl)methanol (2.20 g, 8.39 mmol), Pd/C (10%, 660 mg, 0.62 mmol) in EtOAc (84 mL) and AcOH (4.80 mL, 84 mmol) was placed under hydrogen atmosphere and stirred for 23 h. The reaction mixture was filtered over a pad of celite and washed with EtOAc. The filtrate was concentrated and the residue was diluted in MeOH (100 mL). MP-carbonate (100 mmol, 34.7 g) was added and the mixture was swirled at 40° C. for 1 h. The mixture was filtered, washed with MeOH and the filtrate was concentrated under reduced pressure. The crude residue was purified by normal phase chromatography on basic alumina (eluent: MeOH in CH₂Cl₂ from 0 to 10%). UPLC-MS-4: Rt=0.12 min; MS m/z [M+H]⁺ 173.2.

### Intermediate A8: 5-Methyl-8-oxa-2,5-diazaspiro[3.5]nonane

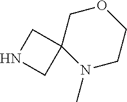

### Step 1: Tert-butyl 5-methyl-8-oxa-2,5-diazaspiro[3.5]nonane-2-carboxylate

A solution of formaldehyde (37-41% aqueous, 296 mg, 3.94 mmol) and tert-butyl-8-oxa-2,5-diazaspiro[3.5]nonane-2-carboxylate (CAS [1251002-01-3], 900 mg, 3.94 mmol) in dichloroethane (24.3 mL) was stirred at 0° C. for 10 min. Then, NaBH(OAc)₃ (1.25 g, 5.91 mmol) was added and the reaction mixture was stirred at 25° C. for 16 h. The RM was poured into a sat. aq. NaHCO₃ solution and extracted with CH₂Cl₂ (2×). The combined organic layer was dried (phase separator) and concentrated to give the title compound which was used without purification in the next step. UPLC-MS-4: Rt=0.44 min; MS m/z [M+H]⁺ 243.3.

### Step 2: 5-Methyl-8-oxa-2,5-diazaspiro[3.5]nonane

Tert-butyl 5-methyl-8-oxa-2,5-diazaspiro[3.5]nonane-2-carboxylate (Step 1, 607 mg, 2.51 mmol) was diluted in CH₂Cl₂ (15 mL) and TFA (1.93 mL, 25.1 mmol) was added. The reaction mixture was stirred at RT for 2 h. The RM was concentrated on the rotavap and under high vacuum. The crude residue was dissolved in MeOH (20 mL), MP-Carbonate (16.6 g, 50.1 mmol) was added and the mixture was stirred at 40° C. for 1 h, filtered, washed with MeOH and concentrated. The crude residue was purified by normal phase chromatography on basic alumina (eluent: MeOH in CH₂Cl₂ from 0 to 10%) to give the title compound. UPLC-MS-4: Rt=0.12 min; MS m/z [M+H]⁺ 143.3.

### Intermediate A9: Tert-butyl 2,2-dimethyl-4-(methylamino)piperidine-1-carboxylate

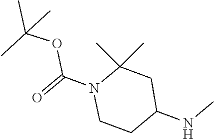 [SMILES: CNC1CCN(C(=O)OC(C)(C)C)C(C)(C)C1]

To a solution of tert-butyl 2,2-dimethyl-4-oxopiperidine-1-carboxylate (2.00 g, 8.80 mmol) in MeOH (20 mL) was added methanamine (2M in MeOH, 8.80 mL, 17.6 mmol) and acetic acid (0.50 mL, 8.80 mmol) and the reaction mixture was stirred at RT for 1 h under nitrogen atmosphere. Sodium triacetoxyborohydride (5.59 g, 26.4 mmol) was added and the RM was stirred at RT overnight. Water (30 mL) was added and the mixture was extracted with EtOAc (2×). The combined organic extracts were washed with water and the water layer was extracted with n-butanol (×2). The combined n-butanol layers were dried and concentrated under reduced pressure. The crude residue was used in the next step without purification. UPLC-MS-2a: Rt=0.66 min; MS m/z [M+H]⁺ 243.2.

### Method-A10 for the synthesis of Intermediate A10: 1-((3R,5S)-3,5-dimethylpiperazin-1-yl)ethan-1-one

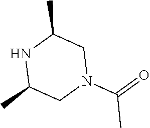

### Step 1: Tert-butyl (2R,6S)-4-acetyl-2,6-dimethylpiperazine-1-carboxylate

To a solution of tert-butyl cis-2,6-dimethylpiperazine-1-carboxylate (1.12 g, 5.23 mmol) in CH₂Cl₂ (52.3 mL) at 0° C. under N₂ atmosphere were added dropwise Et₃N (1.82 mL, 13.1 mmol) followed by acetyl chloride (0.56 mL, 7.84 mmol) in CH₂Cl₂ (100 □L). The reaction mixture was stirred at 0° C. for 1 h. Then HCl (1N) was added, the layers were separated and the organic layer was neutralized with a sat. aq. NaHCO₃ solution, dried (Na₂SO₄), filtered and evaporated to give the title compound as a yellow oil which was used without purification in the next step. UPLC-MS-2b: Rt=0.89 min; MS m/z [M+H]⁺ 257.

### Step 2: 1-((3R,5S)-3,5-dimethylpiperazin-1-yl)ethan-1-one

To a solution of tert-butyl (2R,6S)-4-acetyl-2,6-dimethylpiperazine-1-carboxylate (1.35 g, 5.27 mmol) in 1,4-dioxane (5.27 mL) was added HCl (4N in dioxane, 21 mL) and the reaction mixture was stirred at RT for 2 h. Then, the mixture was lyophilized, the residue was dissolved in MeOH (40 mL), MP-carbonate (21.1 mmol) was added and the mixture was swirled for 15 min, filtered and the residue was washed with MeOH. The filtrated was concentrated under reduced pressure to give the title compound as a yellow oil. UPLC-MS-2b: Rt=0.23 min; MS m/z [M+H]⁺ 157.

Method-A10a: similar to Method-A10 except that in Step 2 TFA was used in CH₂Cl₂ as described in Method-A1.

The following intermediates A1 to A26 were prepared using analogous methods to Method-10 from commercially available reagents.

| | | | Characterizing |
|---|---|---|---|
| Intermediate | Structure | Method and Precursor | data |
| A11 | 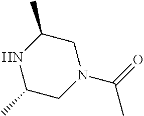 | Using Method-A10 from tert-butyl (2S,6S)-2,6- dimethylpiperazine-1- carboxylate | MS-1; MS m/z [M + H]⁺ 157.1 |
| | 1-((3S,5S)-3,5- | | |
| | dimethylpiperazin-1- | | |
| | yl)ethan-1-one | | |
| A12 | 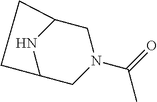 | Using Method-A10 from tert-butyl 3,8- diazabicyclo[3.2.1]octane- 8-carboxylate | MS-1; MS m/z [M + H]⁺ 155.1 |
| | 1-(3,8- | | |
| | diazabicyclo[3.2.1]octan- | | |
| | 3-yl)ethan-1-one | | |
| A13 | 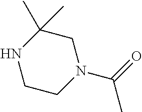 | Using Method-A10 from tert-butyl 2,2- dimethylpiperazine-1- carboxylate | UPLC-MS-2b: Rt = 0.24 min; MS m/z [M + H]⁺ 157.1. |
| | 1-(3,3-dimethylpiperazin- | | |
| | 1-yl)ethan-1-one | | |
| A14 | 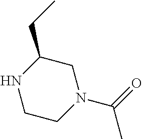 | Using Method-A10 from tert-butyl (S)-2- ethylpiperazine-1- carboxylate | UPLC-MS-2b: Rt = 0.25 min; MS m/z [M + H]⁺ 157.1. |
| | (S)-1-(3-ethylpiperazin-1- | | |
| | yl)ethan-1-one | | |
| A15 | 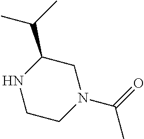 | Using Method-A10 from tert-butyl (S)-2- ethylpiperazine-1- carboxylate | UPLC-MS-2a: Rt = 0.25 min; MS m/z [M + H]⁺ 171.1. |
| | (S)-1-(3- | | |
| | isopropylpiperazin-1- | | |
| | yl)ethan-1-one | | |
| A16 | 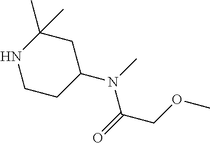 | Using Method-A10 from Intermediate A9 and 2- methoxyacetyl chloride | UPLC-MS-2a: Rt = 0.26 min; MS m/z [M + H]⁺ 215.2. |
| | N-(2,2-dimethylpiperidin- | | |
| | 4-yl)-2-methoxy-N- | | |
| | methylacetamide | | |
| A17 | 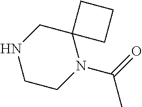 | Using Method-A10 from tert-butyl 5,8- diazaspiro[3.5]nonane-8- carboxylate | UPLC-MS-2f: Rt = 0.21-0.27 min; MS m/z [M + H]⁺ 169.1. |
| | 1-(5,8- | | |
| | diazaspiro[3.5]nonan-5- | | |
| | yl)ethan-1-one | | |
| A18 | 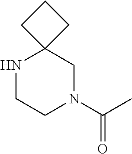 | Using Method-A10 from tert-butyl 5,8- diazaspiro[3.5] nonane-5-carboxylate | UPLC-MS-1e: Rt = 0.24 min; MS m/z [M + H]⁺ 169.1. |
| | 1-(5,8- | | |
| | diazaspiro[3.5]nonan-8- | | |
| | yl)ethan-1-one | | |
| A19 | 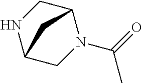 | Using Method-A10a from tert-butyl (1R,4R)-2,5- diazabicyclo[2.2.1]heptane- 2-carboxylate | UPLC-MS-1a: Rt = 0.25 min; MS m/z [M + H]⁺ 141.1. |
| | 1-((1R,4R)-2,5- | | |
| | diazabicyclo[2.2.1 ]heptan- | | |
| | 2-yl)ethan-1-one | | |
| A20 | 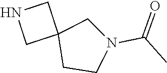 | Using Method-A10 from tert-butyl 2,6- diazaspiro[3,4]octane- carboxylate | UPLC-MS-1a: Rt = 0.25 min; MS m/z [M + H]⁺ 301. |
| | 1-(2,6- | | |
| | diazaspiro[3.4]octan-6- | | |
| | yl)ethan-1-one | | |
| A21 | 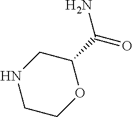 | Using Method-A10 step 2 from tert-butyl (R)-2- carbamoylmorpholine-4- carboxylate | ¹H NMR (400 MHZ, DMSO-d₆) δ 7.52 (br s, 1H), 7.44 (br s, 1H), 4.23 (dd, 1H), 4.14-3.94 (m, 1H), 4.01 (m, 1H), 3.84 (td, 1H), 3.55 (d, 1H), 3.17 (d, |
| | (R)-morpholine-2- | | 1H), 3.05-2.83 (m, |
| | carboxamide | | 2H). |
| A22 | 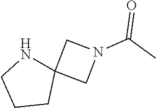 | Using Method-A10a from tert-butyl 2,5- diazaspiro[3.4]octane-5- carboxylate (CAS [1086398-04-0]) | UPLC-MS-1a: Rt = 0.17 min; MS m/z [M + H]⁺ 155.2. |
| | 1-(2,5- | | |
| | diazaspiro[3.4]octan-2- | | |
| | yl)ethan-1-one | | |
| A23 | 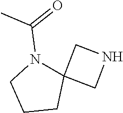 | Using Method-A10a from tert-butyl 2,5- diazaspiro[3.4]octane-2- carboxylate (CAS [1086398-02-8]) | UPLC-MS-4: Rt = 0.13 min; MS m/z [M + H]⁺ 155.2. |
| | 1-(2,5- | | |
| | diazaspiro[3.4]octan-5- | | |
| | yl)ethan-1-one | | |
| A24 | 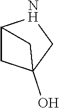 | Using Method-A10 step 2 from tert-butyl 4-hydroxy-2- azabicyclo[2.1.1 ]hexane-2- carboxylate | UPLC-MS-4: Rt = 0.12 min; MS m/z [M + H]⁺ 100.6. |
| | 2-azabicyclo[2.1.1]hexan- | | |
| | 4-ol | | |
| A25 | 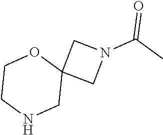 | Using Method-A10a from tert-butyl 5-oxa-2,8- diazaspiro[3.5]nonane-8- carboxylate (CAS: [1251005-61-4]) | MS-1: MS m/z [M + H]⁺ 171.4. |
| | 1-(5-oxa-2,8- | | |
| | diazaspiro[3.5]nonan-2- | | |
| | yl)ethan-1-one | | |
| A26 | 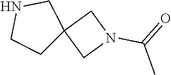 | Using Method-A10 from tert-butyl 2,6- diazaspiro[3.4]octane-6- carboxylate hydrochloride (CAS [1841081-35-3]) | UPLC-MS-4: Rt = 0.12 min; MS m/z [M + H]⁺ 155.2. |
| | 1-(2,6- | | |
| | diazaspiro[3.4]octan-2- | | |
| | yl)ethan-1-one | | |

### Intermediate A27: 8-(2-Methoxyethyl)-5,8-diazaspiro[3.5]nonane

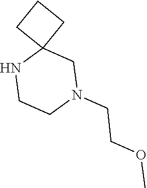

### Step 1: Tert-butyl 8-(2-methoxyethyl)-5,8-diazaspiro[3.5]nonane-5-carboxylate

In an ACE tube, to a solution of tert-butyl 5,8-diazaspiro[3.5]nonane-5-carboxylate (2.00 g, 8.84 mmol) in CH₃CN (60 mL) was added 2-bromoethyl methyl ether (1.33 mL, 14.1 mmol) and Et₃N (3.67 mL, 26.5 mmol) and the reaction mixture was stirred at reflux for 5.5 h. After completion of the reaction, the reaction mixture was poured into an aq. sat. NaHCO₃ solution and extracted with CH₂Cl₂ (×2). The combined organic extracts were dried (phase separator) and concentrated to give the title compound as a brown oil which was used without purification in the next step. MS-1: MS m/z [M+H]⁺ 285.4.

### Step 2: 8-(2-Methoxyethyl)-5,8-diazaspiro[3.5]nonane

To a solution of tert-butyl 8-(2-methoxyethyl)-5,8-diazaspiro[3.5]nonane-5-carboxylate (Step 1, 2.58 g, 8.82 mmol) in 1,4-dioxane (9.0 mL) was added HCL (4N in dioxane, 22.0 mL, 88 mmol) and the reaction mixture was stirred at RT for 2 h. The RM was frozen and lyophilized to give the title compound as a hydrochloride salt (white solid). The material was dissolved in MeOH (60 mL), MP-carbonate (35.3 mmol, 11.65 g) was added and the mixture was swirled at RT for 30 min. The mixture was filtered, the precipitate was washed with MeOH and the filtrate was concentrated under reduced pressure to give the title compound as a brown oil. MS-1: MS m/z [M+H]⁺ 185.2.

### Intermediate A28: 8-Methyl-5,8-diazaspiro[3.5]nonan-7-one

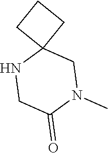

### Step 1: Benzyl 7-oxo-5,8-diazaspiro[3.5]nonane-5-carboxylate

Benzyl chloroformate (2.85 mL, 19.2 mmol) was added to a solution of 5,8-diazaspiro[3.5]nonan-7-one (CAS [1557629-00-1], 2.45 g, 17.5 mmol) and DIPEA (6.42 mL, 36.7 mmol) in CH₂Cl₂ (70 mL) at RT. After 2 h, the reaction mixture was diluted with a sat. aq. NaHCO₃ solution and extracted with CH₂Cl₂ (2×). The combined organic layers were dried (Na₂SO₄) and concentrated. The crude residue was purified by normal phase chromatography (eluent: heptane/(20:1 EtOAc/MeOH), 0 to 100%) to give the title compound as a colorless oil. UPLC-MS-2a: Rt=0.90 min; MS m/z [M+H]⁺ 275.2.

### Step 2: Benzyl 8-methyl-7-oxo-5,8-diazaspiro[3.5]nonane-5-carboxylate

NaH (95%, 83 mg, 3.30 mmol) was added to a solution of benzyl 7-oxo-5,8-diazaspiro[3.5]nonane-5-carboxylate (Step 1, 831 mg, 3.00 mmol) in DMF (15 mL) at 0° C. under nitrogen atmosphere. After stirring for 10 min, iodomethane (0.28 mL, 4.50 mmol) was added and the reaction was allowed to warm to RT. After 1 h, the mixture was diluted with a sat. aq. NaHCO₃ solution and extracted with EtOAc (2×). The combined organic layers were washed with H₂O/brine (9:1) and brine, dried (Na₂SO₄), filtered and concentrated. The crude residue was purified by normal phase chromatography (eluent: heptane/(20:1 EtOAc/MeOH), 0 to 100%) to give the title compound as a colorless oil. UPLC-MS-2a: Rt=0.93 min; MS m/z [M+H]⁺ 289.2.

### Step 3: 8-Methyl-5,8-diazaspiro[3.5]nonan-7-one

A mixture of benzyl 8-methyl-7-oxo-5,8-diazaspiro[3.5]nonane-5-carboxylate (Step 2, 1.17 g, 3.85 mmol) and 10% Pd/C (205 mg) in EtOH (19 mL) was placed under a hydrogen atmosphere (ambient pressure) and stirred at RT for 90 min. The reaction mixture was filtered through a celite pad and washed with EtOH. The filtrate was concentrated to give the title compound which was used without purification in the next step. NMR (400 MHz, DMSO-d₆) δ 3.23 (s, 2H), 3.18 (s, 2H), 2.80 (s, 3H), 2.66 (br s, 1H), 1.84-1.91 (m, 4H), 1.71-1.84 (m, 2H).

### Intermediate A29: 1-(2,5-Diazaspiro[3.5]nonan-2-yl)ethan-1-one

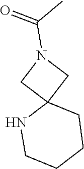

### Step 1: 5-Benzyl 2-(tert-butyl) 2,5-diazaspiro[3.5]nonane-2,5-dicarboxylate

Benzyl chloroformate (1.44 mL, 9.60 mmol) was added to a solution of 5,8-diazaspiro[3.5]nonan-7-one (CAS [1246034-93-4], 1.91 g, 8.00 mmol) and K₂CO₃ (3.32 g, 24.0 mmol) in THF (40 mL) at RT. After 18 h, the reaction mixture was diluted with a sat. aq. NaHCO₃ solution and extracted with EtOAc (2×). The combined organic layers were washed with brine, dried (Na₂SO₄), filtered and concentrated. The crude residue was purified by normal phase chromatography (eluent: heptane/(20:1 EtOAc/MeOH), 0 to 100%) to give the title compound as a colorless oil. UPLC-MS-2a: Rt=1.24 min; MS m/z [M+H]⁺ 361.2.

### Step 2: Benzyl 2-acetyl-2,5-diazaspiro[3.5]nonane-5-carboxylate

TFA (11.4 mL, 147 mmol) was added to a solution of 5-benzyl 2-(tert-butyl) 2,5-diazaspiro[3.5]nonane-2,5-dicarboxylate (Step 1, 2.95 g, 7.37 mmol) in CH₂Cl₂ (74 mL) at RT. After 2 h, the reaction mixture was concentrated. The residue was diluted with a sat. aq. NaHCO₃ and extracted with EtOAc (4×). The combined organic layers were washed with brine, dried (Na₂SO₄), filtered and concentrated. The crude intermediate was dissolved in dioxane (37 mL) and H₂O (37 mL), treated with K₂CO₃ (3.05 g, 22.1 mmol), stirred for 5 min and then acetic anhydride (0.70 mL, 7.37 mmol) was added to the reaction mixture. After 1 h, the mixture was diluted with a sat. aq. NaHCO₃ and extracted with EtOAc (2×). The combined organic layers were washed with brine, dried (Na₂SO₄), filtered and concentrated. The crude residue was purified by normal phase chromatography (eluent: heptane/(20:1 EtOAc/MeOH), 0 to 100%) to give the title compound as a colorless oil. UPLC-MS-2a: Rt=0.93 min; MS m/z [M+H]⁺ 303.2.

### Step 3: 1-(2,5-Diazaspiro[3.5]nonan-2-yl)ethan-1-one

A mixture of benzyl 2-acetyl-2,5-diazaspiro[3.5]nonane-5-carboxylate (Step 2, 350 mg, 1.04 mmol) and 10% Pd/C (111 mg) in EtOH (10 mL) was placed under a hydrogen atmosphere (ambient pressure) and stirred at RT for 90 min. The reaction mixture was filtered through a celite pad and washed with EtOH. The filtrate was concentrated to give the title compound which was used without purification in the next step. ¹H NMR (400 MHz, DMSO-d₆) δ 3.83 (m, 1H), 3.75 (m, 1H), 3.55 (m, 1H), 3.48 (m, 1H), 2.53-2.65 (m, 3H), 1.74 (s, 3H), 1.54 (m, 2H), 1.46 (m, 2H), 1.35 (m, 2H). c

### Intermediate A30: 2-(Trimethylsilyl)ethyl 5,8-diazaspiro[3.5]nonane-8-carboxylate

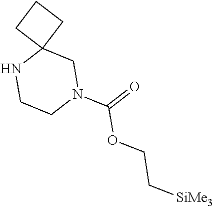 [SMILES: C[Si](C)(C)CCOC(=O)N1CCNC2(CCC2)C1]

### Step 1: 5,8-Diazaspiro[3.5]nonane

A mixture of tert-butyl 5,8-diazaspiro[3.5]nonane-5-carboxylate (23.0 g, 102 mmol) and HCl (4M in 1,4-dioxane, 254 mL, 1016 mmol) was stirred at RT for 16 h. The reaction mixture was concentrated under vacuum to give the title compound as a hydrochloride salt (white solid) which was used without purification in the next step. ¹H NMR (400 MHz, DMSO-d₆) δ 10.14 (d, 4H), 3.47 (s, 2H), 3.27-3.18 (m, 4H), 2.43-2.19 (m, 4H), 1.96-1.81 (m, 2H).

### Step 2: 2-(Trimethylsilyl)ethyl 5,8-diazaspiro[3.5]nonane-8-carboxylate

Under inert atmosphere, to a stirred solution of 5,8-diazaspiro[3.5]nonane hydrochloride (Step 1, 102 mmol) in CH₂Cl₂ (400 mL) were added DIPEA (142 mL, 816 mmol) and 2,5-dioxopyrrolidin-1-yl (2-(trimethylsilyl)ethyl) carbonate (26.4 g, 102 mmol). The reaction mixture was stirred at RT for 16 h. The RM was quenched with a sat. aq. NaHCO₃ solution and extracted with CH₂Cl₂ (×2). The combined organic layers were washed with a sat. aq. NaHCO₃ solution, dried (Na₂SO₄), filtered and concentrated under vacuum. The crude residue was purified by normal phase chromatography (eluent: MeOH in CH₂Cl₂ 0 to 10%) to give the title compound as a yellow oil. ¹H NMR (400 MHz, DMSO-d₆) δ 4.13-4.05 (m, 2H), 3.23 (s, 4H), 2.59-2.53 (m, 2H), 1.83-1.79 (m, 2H), 1.74-1.64 (m, 4H), 0.98-0.90 (m, 2H), 0.02 (s, 9H).

### Intermediate A31: 8-Benzyl-5,8-diazaspiro[3.5]nonane

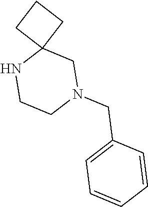 [SMILES: c1ccc(CN2CCNC3(CCC3)C2)cc1]

### Step 1: 8-Benzyl-5,8-diazaspiro[3.5]nonan-9-one

To a solution of benzyltriethylammonium chloride (212 mg, 0.93 mmol) in CH₂Cl₂ (50.0 mL) was added N-benzylethylenediamine (4.80 mL, 31.0 mmol) followed by chloroform (5.00 mL, 62.0 mmol) and cyclobutanone (4.68 mL, 62.0 mmol). The reaction mixture was cooled to 0° C. and 30% aqueous NaOH solution (25.0 mL, 313 mmol) was added dropwise. Then the reaction mixture was stirred at RT for 60 h. The RM was diluted with water and extracted twice with CH₂Cl₂. The combined organic layers were washed with brine, dried over Na₂SO₄, filtered, and concentrated under vacuum. The crude residue was purified by preparative HPLC (RP-HPLC-1: mobile phase: A: water+0.1% TFA, B: acetonitrile; gradient: 10 to 50% B in 25 min). Product containing fractions were combined, basified with a sat. aq. NaHCO₃ solution, and extracted twice with EtOAc. The combined organic layers were washed with brine, dried (Na₂SO₄), filtered, and concentrated under vacuum to give the title compound as an oil. UPLC-MS-2a: Rt=0.58 min; MS m/z [M+H]⁺ 231.2.

### Step 2: 8-Benzyl-5,8-diazaspiro[3.5]nonane

Under inert atmosphere, to a solution of LiAlH₄ (1M in THF, 19.0 mL, 19.0 mmol) in THF (60.0 mL) was added at RT a solution of 8-benzyl-5,8-diazaspiro[3.5]nonan-9-one (Step 1, 4.00 g, 16.5 mmol) in THF (40.0 mL) dropwise with stirring. The reaction mixture was stirred at RT for 20 h. The RM was diluted with THF (150 mL), water (0.72 mL) was added followed by NaOH (3.5M aq. solution, 0.72 mL) and then water (2.00 mL). The RM was stirred at RT for 15 min, dried (Na₂SO₄), filtered, and concentrated under vacuum. The crude residue was purified by normal phase chromatography (eluent: MeOH in CH₂Cl₂ 0 to 10%) to give the title compound as an oil. UPLC-MS-2a: Rt=0.65 min; MS m/z [M+H]⁺ 217.2.

### Intermediate A32: (S)-1-Benzyl-3-ethyl-3-methylpiperazine

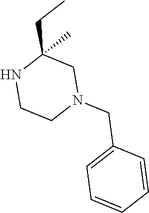 [SMILES: CC[C@@]1(C)CN(Cc2ccccc2)CCN1]

### Step 1: (S)-1-Benzyl-3-ethyl-3-methylpiperazin-2-one and (R)-1-benzyl-3-ethyl-3-methylpiperazin-2-one

To an ice-cooled solution of benzyltriethylammonium chloride (14.7 g, 64.6 mmol) in CH₂Cl₂ (2.00 L) was added N-benzylethylenediamine (200 mL, 1.29 mol) followed by chloroform (208 mL, 2.58 mol), butan-2-one (231 mL, 2.58 mol) and 30% aqueous NaOH solution (1.03 L, 12.9 mol). Then the reaction mixture was warmed up to RT and stirred for 3 days. The RM was diluted with water and extracted twice with CH₂Cl₂. The combined organic layers were washed with brine, dried (MgSO₄), filtered, and concentrated under vacuum. The residue was purified twice by normal phase chromatography (eluent: MeOH in CH₂Cl₂ 0 to 7%) to give the enantiomeric mixture of the title compound as a pale yellow oil. The enantiomers were separated by chiral C-SFC-12 (mobile phase: CO₂/[MeOH+0.025% NH₃] 75/25) to give (S)-1-benzyl-3-ethyl-3-methylpiperazin-2-one as the first eluting enantiomer: (C-SFC-40 (mobile phase: 5 to 40% [IPA+0.05% DEA] in CO₂): Rt=3.97 min); UPLC-MS-2a: Rt=0.55 min; MS m/z [M+H]⁺ 233.3 and (R)-1-benzyl-3-ethyl-3-methylpiperazin-2-one as the second eluting enantiomer: (C-SFC-40 (mobile phase: 5 to 40% [IPA+0.05% DEA] in CO₂): Rt=4.18 min); UPLC-MS-2a: Rt=0.54 min; MS m/z [M+H]⁺ 233.3.

### Step 2: (S)-1-Benzyl-3-ethyl-3-methylpiperazine

To LiAlH₄ (2M in THF, 50.2 mL, 100 mmol) under a nitrogen atmosphere was added a solution of (S)-1-benzyl-3-ethyl-3-methylpiperazin-2-one (Step 1 first eluting enantiomer, 15.7 g, 66.9 mmol) in THF (335 mL) dropwise with stirring at RT (slightly exothermic (˜35° C.)). The reaction mixture was stirred at 60° C. for 2 h. The RM was slowly quenched by careful addition of water (2.00 mL) at 0° C. followed by 15% aqueous NaOH solution (2.00 mL) then water (6.00 mL). To the suspension was added Na₂SO₄, the mixture was filtered and washed with EtOAc. The combined organic layers were concentrated under vacuum and the residue was diluted with EtOAc and water and extracted with EtOAc (2×250 mL). The combined organic layers were washed with 1.5M aq. potassium sodium tartrate (Rochelle salt) then brine (100 mL), dried (Na₂SO₄), filtered, and concentrated under vacuum. The residue was purified by normal phase chromatography (eluent: MeOH in CH₂Cl₂ 0 to 20%) to give the title compound as a yellow oil. UPLC-MS-2a: Rt=0.69 min; MS m/z [M+H]⁺ 219.3.

### Intermediate A33: (R)-1-Benzyl-3-ethyl-3-methylpiperazine

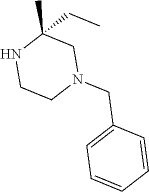 [SMILES: CC[C@]1(C)CN(Cc2ccccc2)CCN1]

The title compound was prepared by a method similar to that of (S)-1-benzyl-3-ethyl-3-methylpiperazine (Intermediate A32) using (R)-1-benzyl-3-ethyl-3-methylpiperazin-2-one (Intermediate A32, Step 1 second eluting enantiomer) instead of (S)-1-benzyl-3-ethyl-3-methylpiperazin-2-one. UPLC-MS-4: Rt=0.38 min; MS m/z [M+H]⁺ 219.3.

### Intermediate A34: (R)-1-Benzyl-3-(difluoromethyl)-3-methylpiperazine

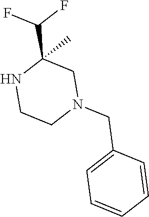 [SMILES: C[C@]1(C(F)F)CN(Cc2ccccc2)CCN1]

### Step 1: (R)-1-Benzyl-3-(difluoromethyl)-3-methylpiperazin-2-one

To an ice-cooled solution of benzyl(triethyl)ammonium chloride (152 g, 666 mmol) in CH₂Cl₂ (10 L) was added N-benzylethylenediamine (1000 g, 6.66 mol), 1,1-difluoropropan-2-one (1.25 kg, 13.2 mol) followed by NaOH (13 M, 5.12 L) and finally CHCl₃ (1.59 kg, 13.3 mol). The mixture was stirred at 15° C. for 18 h. The mixture was poured into water. The aqueous phase was extracted twice with CH₂Cl₂. The combined organic phases were washed with brine, dried (Na₂SO₄), filtered and concentrated in vacuum. The crude residue was purified by normal phase chromatography (eluent: Petroleum ether/Ethyl acetate; 100/1 to 10/1) to give the enantiomeric mixture of the title compound as a pale yellow gum. The enantiomers were separated by chiral C-SFC-41 (mobile phase: [MeOH+0.1% NH₄OH] in CO₂ 35%) to give (S)-1-benzyl-3-(difluoromethyl)-3-methylpiperazin-2-one as the first eluting enantiomer: C-SFC-42 (mobile phase: [MeOH+0.05% DEA] in CO₂ 5 to 40%): Rt=1.58 min, UPLC-MS-2a: Rt=0.73 min; MS m/z [M+H]⁺ 255.2 and (R)-1-benzyl-3-(difluoromethyl)-3-methylpiperazin-2-one as the second eluting enantiomer: C-SFC-42 (mobile phase: [MeOH+0.05% DEA] in CO₂ 5 to 40%): Rt=2.04 min, UPLC-MS-2a: Rt=0.40 min; MS m/z [M+H]⁺ 255.2.

### Step 2: (R)-1-Benzyl-3-(difluoromethyl)-3-methylpiperazine

To an ice-cooled solution of (R)-1-benzyl-3-(difluoromethyl)-3-methylpiperazin-2-one (Step 1 second eluting enantiomer, 151 g, 595 mmol) in THF (900 mL) was added dropwise under inert atmosphere at 0° C. BH₃-Me₂S (10 M in THF, 595 mL). The mixture was stirred at 75° C. for 20 h. After completion, the reaction mixture was cooled to 0° C. and quenched by adding MeOH (600 ml) and HCl (4N, 400 mL) until pH 3. The reaction mixture was stirred at 50° C. for 12 h and was concentrated under reduced pressure. The solution was poured into ice-cooled mixture of 15% NaOH (600 mL) and CH₂Cl₂/MeOH (10/1) to adjust the pH around 14. The layers were separated, the aqueous layer was bac-extracted with CH₂Cl₂ and the combined organic phases were dried (Na₂SO₄), filtered and evaporated. The crude residue was purified by normal phase chromatography (eluent: Petroleum ether/EtOAc 100/1 to 20/1) to give the title compound as a yellow oil. UPLC-MS-4: Rt=0.40 min; MS m/z [M+H]⁺ 241.3.

### Intermediate A35: (S)-1-benzyl-3-(difluoromethyl)-3-methylpiperazine

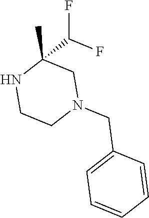 [SMILES: C[C@@]1(C(F)F)CN(Cc2ccccc2)CCN1]

The title compound was prepared by a method similar to that of (R)-1-benzyl-3-(difluoromethyl)-3-methylpiperazine (Intermediate A34) using (S)-1-benzyl-3-(difluoromethyl)-3-methylpiperazin-2-one (Intermediate A34, Step 1 first eluting enantiomer) instead of (R)-1-benzyl-3-(difluoromethyl)-3-methylpiperazin-2-one. UPLC-MS-4: Rt=0.42 min; MS m/z [M+H]⁺; 241.2.

### Intermediate A36: (S)-1-Benzyl-3-(difluoromethyl)-3-ethylpiperazine

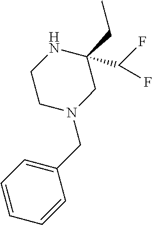 [SMILES: CC[C@@]1(C(F)F)CN(Cc2ccccc2)CCN1]

### Step 1: (S)-1-Benzyl-3-(difluoromethyl)-3-ethylpiperazin-2-one and (R)-1-benzyl-3-(difluoromethyl)-3-ethylpiperazin-2-one

To an ice-cooled solution of benzyltriethylammonium chloride (0.63 g, 2.74 mmol) in CH₂Cl₂ (85 mL) were added N-benzylethylenediamine (8.50 mL, 54.9 mmol), 1,1-difluorobutan-2-one (53% in THF, 22.4 g, 110 mmol) followed by sodium hydroxide 30% (43.9 mL, 549 mmol) and finally chloroform (8.85 mL, 110 mmol). The mixture was stirred and allowed to slowly reach RT for 16 h. The mixture was diluted with water and extracted twice with CH₂Cl₂. The combined organic phase was washed with brine, dried (MgSO₄), filtered and evaporated under reduced pressure to give the enantiomeric mixture of the title compound. The enantiomers were separated by chiral C-SFC-45 (mobile phase: 20% EtOH in CO₂) to give (S)-1-benzyl-3-(difluoromethyl)-3-ethylpiperazin-2-one as the first eluting enantiomer: C-SFC-8 (mobile phase: CO₂/[MeOH+0.025% NH₃] 80/20): Rt=1.25 min, UPLC-MS-4: Rt=0.73 min; MS m/z [M+H]⁺ 269.3 and (R)-1-benzyl-3-(difluoromethyl)-3-methylpiperazin-2-one as the second eluting enantiomer: C-SFC-8 (mobile phase: CO₂/[MeOH+0.025% NH₃] 80/20): Rt=1.71 min, UPLC-MS-4: Rt=0.74 min; MS m/z [M+H]⁺ 269.1.

### Step 2: (S)-1-benzyl-3-(difluoromethyl)-3-ethylpiperazine

To a solution of (S)-1-benzyl-3-(difluoromethyl)-3-ethylpiperazin-2-one (Step 1 first eluting enantiomer, 2.00 g, 7.45 mmol) in THF (3.30 mL) was added BH₃·THF (1M in THF, 74.5 mL, 74.5 mmol) at 0° C. The mixture was stirred at 0° C. for 15 min, at RT for 15 min and then at 75° C. for 48 h. After cooling to 0° C., the RM was quenched carefully with MeOH (30 mL) and HCl (4N, 20 mL) until pH 3. The mixture was stirred at 0° C. for 2 h and then was concentrated. The aqueous solution was poured into an ice-cooled mixture of 15% NaOH (16 mL) to reach pH 14. The aqueous phase was extracted with CH₂Cl₂/MeOH (9/1) (×3). The combined organic layer was dried (MgSO₄), filtered and evaporated. The crude residue was purified by normal phase chromatography (eluent: TBME in n-heptane, 0 to 100%) to give the title compound. UPLC-MS-4: Rt=0.49 min; MS m/z [M+H]⁺ 255.3.

### Intermediate A37: (R)-1-Benzyl-3-(methoxymethyl)-3-methylpiperazine

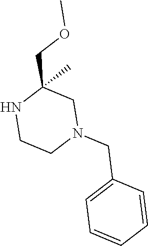 [SMILES: COC[C@@]1(C)CN(Cc2ccccc2)CCN1]

### Step 1: (S)-1-Benzyl-3-(methoxymethyl)-3-methylpiperazin-2-one

To an ice-cooled solution of benzyltriethylammonium chloride (7.76 g, 34.0 mmol) in CH₂Cl₂ (600 mL) was added N-benzylethylenediamine (102 mL, 0.68 mol), methoxyacetone (62.7 mL, 0.68 mol) followed by NaOH 30% (545 mL, 6.81 mol) and finally CHCl₃ (110 mL, 1.36 mol). The reaction mixture was warmed up to RT and stirred for 18 h. The reaction mixture was diluted with water and extracted twice with CH₂Cl₂. The combined organic layers were washed with brine, dried (Na₂SO₄), filtered and concentrated under vacuum. The crude residue was purified by normal phase chromatography (eluent: MeOH in CH₂Cl₂ 0 to 10%) to give the enantiomeric mixture of the title compound as a pale yellow oil. The enantiomers were separated by chiral C-HPLC-17 (mobile phase: CH₃CN+0.2% NH₃) to give (S)-1-benzyl-3-(methoxymethyl)-3-methylpiperazin-2-one as the first eluting enantiomer: C-HPLC-18 (mobile phase: CH₃CN+0.2% NH₃: Rt=4.49 min, UPLC-MS-4: Rt=0.25 min; MS m/z [M+H]⁺ 249.2 and (R)-1-benzyl-3-(methoxymethyl)-3-methylpiperazin-2-one as the second eluting enantiomer: C-HPLC-18 (mobile phase: CH₃CN+0.2% NH₃: Rt=6.18 min, UPLC-MS-4: Rt=0.25 min; MS m/z [M+H]⁺ 249.2.

### Step 2: (R)-1-benzyl-3-(methoxymethyl)-3-methylpiperazine

To a solution of (S)-1-benzyl-3-(methoxymethyl)-3-methylpiperazin-2-one (Step 1 first eluting enantiomer, 41.5 g, 167 mmol) in THF (500 mL) was added LiAlH₄ (2M in THF, 100 mL, 201 mmol) under a nitrogen atmosphere. The reaction mixture was stirred at 60° C. for 30 min. The reaction was slowly quenched by careful addition of Rochelle's salt (potassium sodium tartrate) and extracted with CH₂Cl₂ (×2). The combined organic extracts were washed with a sat. aq. NaHCO₃ solution, dried (Na₂SO₄), filtered and concentrated under vacuum. The crude residue was purified by normal phase chromatography (eluent: MeOH in CH₂Cl₂ 0 to 10%). The fractions containing the desired material were concentrated and the residue was purified again by normal phase chromatography (eluent: (MeOH/NH₄OH 80/20) in CH₂Cl₂ 0 to 10%) to give the title compound as a colorless oil. UPLC-MS-4: Rt=0.37 min; MS m/z [M+H]⁺ 235.3.

### Intermediate A37-rac: 1-Benzyl-3-(methoxymethyl)-3-methylpiperazine

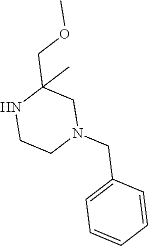 [SMILES: COCC1(C)CN(Cc2ccccc2)CCN1]

The title compound was prepared by a method similar to Step 2 of Intermediate A37 using racemic 1-benzyl-3-(methoxymethyl)-3-methylpiperazin-2-one (Intermediate A37 Step 1) instead of (S)-1-benzyl-3-(methoxymethyl)-3-methylpiperazin-2-one. UPLC-MS-4: Rt=0.37 min; MS m/z [M+H]⁺ 235.3.

### Intermediate A38: (S)-1-Benzyl-3-(2-methoxyethyl)-3-methylpiperazine

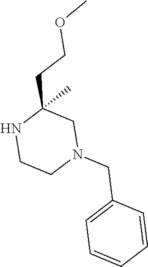 [SMILES: COCC[C@@]1(C)CN(Cc2ccccc2)CCN1]

### Step 1: (S)-1-Benzyl-3-(2-methoxyethyl)-3-methylpiperazin-2-one

To an ice-cooled solution of benzyltriethylammonium chloride (0.58 g, 2.53 mmol) in CH₂Cl₂ (50 mL) was added N-benzylethylenediamine (7.60 mL, 50.6 mmol), NaOH 30% (40.5 mL, 506 mmol) and 4-methoxy-2-butanone (5.17 g, 50.6 mmol) followed by CHCl₃ (8.16 mL, 101 mmol). The reaction mixture was warmed up to RT and stirred for 20 h. The RM was diluted with water and extracted twice with CH₂Cl₂. The combined organic layers were washed with brine, dried (Na₂SO₄), filtered and concentrated under vacuum. The crude residue was purified by normal phase chromatography (eluent: MeOH in CH₂Cl₂ 0 to 8.3%) to give the enantiomeric mixture of the title compound as a pale yellow oil. The enantiomers were separated by chiral C-SFC-13 (mobile phase: [IPA+0.1% NH₄OH] in CO₂ 35%) to give (S)-1-benzyl-3-(2-methoxyethyl)-3-methylpiperazin-2-one as the first eluting enantiomer: C-SFC-14 (mobile phase: [MeOH+0.05% DEA] in CO₂ 5 to 40%): Rt=5.17 min, UPLC-MS-4: Rt=0.25 min; MS m/z [M+H]⁺ 263.1 and (R)-1-benzyl-3-(2-methoxyethyl)-3-methylpiperazin-2-one as the second eluting enantiomer: C-SFC-14 (mobile phase: [MeOH+0.05% DEA] in CO₂ 5 to 40%): Rt=5.55 min, UPLC-MS-4: Rt=0.25 min; MS m/z [M+H]⁺ 263.1.

### Step 2: (S)-1-Benzyl-3-(2-methoxyethyl)-3-methylpiperazine

To a solution of (S)-1-benzyl-3-(2-methoxyethyl)-3-methylpiperazin-2-one (Step 1 first eluting enantiomer, 3.10 g, 11.8 mmol) in THF (50 mL) was added LiAlH₄ (2M in THF, 11.8 mL, 23.6 mmol) under a nitrogen atmosphere. The reaction mixture was stirred at 60° C. for 1 h. The reaction was slowly quenched by careful addition of Rochelle's salt (potassium sodium tartrate) and extracted with CH₂Cl₂ (×2). The combined organic extracts were washed with a sat. aq. NaHCO₃ solution, dried (Na₂SO₄), filtered and concentrated under vacuum. The crude residue was purified by normal phase chromatography (eluent: MeOH in CH₂Cl₂ 0 to 10%) to give the title compound as a yellow oil. UPLC-MS-4: Rt=0.32 min; MS m/z [M+H]⁺ 249.9.

### Intermediate A39: (S)-1-Benzyl-2,5,5-trimethylpiperazine

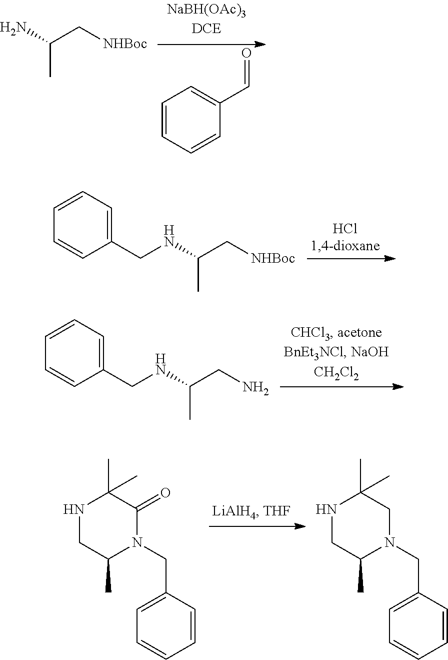 [SMILES: C[C@H](N)CNC(=O)OC(C)(C)C ; O=Cc1ccccc1 ; C[C@@H](CNC(=O)OC(C)(C)C)NCc1ccccc1 ; C[C@@H](CN)NCc1ccccc1 ; C[C@H]1CNC(C)(C)C(=O)N1Cc1ccccc1 ; C[C@H]1CNC(C)(C)CN1Cc1ccccc1]

### Step 1: Tert-butyl (S)-(2-(benzylamino)propyl)carbamate

To a solution of tert-butyl (S)-(2-aminopropyl)carbamate hydrochloride (3.58 g, 17.0 mmol) in MeOH (150 mL) was added MP-carbonate (22.4 g, 17.0 mmol) and the mixture was stirred at 40° C. for 1 h. The mixture was filtered and the filtrate was concentrated in vacuo. The residue was dissolved in DCE (300 mL), benzaldehyde (1.66 mL, 16.4 mmol) was added and the mixture was stirred at RT for 1 h prior to the addition of sodium triacetoxyborohydride (6.93 g, 32.7 mmol) under nitrogen atmosphere. The reaction mixture was then stirred at RT overnight. The RM was quenched with a sat. aq. NaHCO₃ solution and extracted with EtOAc. The combined organic layers were washed with brine, dried (Na₂SO₄), filtered and concentrated in vacuo. The crude residue was purified by normal phase chromatography (eluent: MeOH in CH₂Cl₂ 0 to 20%) to give the title compound as a yellow oil. UPLC-MS-4: Rt=0.41 min; MS m/z [M+H]⁺ 265.2.

### Step 2: (S)—N²-Benzylpropane-1,2-diamine

To a solution of tert-butyl (S)-(2-(benzylamino)propyl)carbamate (Step 1, 3.15 g, 8.35 mmol) was added HCl (4M in 1,4-dioxane, 12.5 mL, 50.1 mmol) at 0° C. and the reaction mixture was stirred at RT overnight. The solvent was evaporated and co-evaporated with CH₂Cl₂ (×2). The residue was dissolved in MeOH (100 mL) and MP-carbonate (25.0 g, 8.58 mmol) was added. The mixture was stirred at 40° C. for 1 h, filtered and the filtrate was concentrated in vacuo to give the title compound as a yellow oil. ¹H NMR (400 MHz, DMSO-d₆) δ 7.39-7.15 (m, 5H), 3.81-3.59 (m, 2H), 3.30 (br s, 1H), 3.17 (s, 1H), 2.58-2.36 (m, 2H), 2.01 (br s, 2H), 0.95 (d, 3H).

### Step 3: (S)-1-Benzyl-3,3,6-trimethylpiperazin-2-one

To a solution of N-benzyl-N,N-diethylethanaminium chloride (90.0 mg, 0.39 mmol) in CH₂Cl₂ (80 mL) were added (S)—N²-benzylpropane-1,2-diamine (Step 2, 1.30 g, 7.91 mmol), chloroform (1.28 mL, 15.8 mmol) and propan-2-one (1.17 mL, 15.8 mmol) followed by NaOH (30% aq. solution, 6.33 mL, 79 mmol) at 0° C. The reaction mixture was stirred at RT overnight. The RM was diluted with water, extracted with CH₂Cl₂ (×2) and the combined organic layers were washed with brine, dried (MgSO₄), filtered and concentrated in vacuo. The crude residue was purified by normal phase chromatography (eluent: MeOH in CH₂Cl₂ 0 to 20%) to give the title compound as a beige solid. UPLC-MS-4: Rt=0.28 min; MS m/z [M+H]⁺ 233.1.

### Step 4: (S)-1-Benzyl-2,5,5-trimethylpiperazine

To a solution of (S)-1-benzyl-3,3,6-trimethylpiperazin-2-one (Step 3, 760 mg, 2.94 mmol) in THF (15.0 mL) under a nitrogen atmosphere was added LiAlH₄ (2M in THF, 4.42 mL, 8.83 mmol) dropwise at 0° C. The reaction mixture was slowly allowed to reach RT and stirred at RT for 3.5 h. The RM was diluted with CH₂Cl₂ and was quenched carefully by addition of NaOH (1M aqueous solution) at 0° C. The mixture was stirred until the organic layer became clear and the white precipitate was filtered off. The filtrate was extracted with CH₂Cl₂ and the combined organic layers were dried (Na₂SO₄), filtered and concentrated in vacuo to give the title compound as a colorless oil. UPLC-MS-4: Rt=0.45 min; MS m/z [M+H]⁺ 219.3.

### Intermediate A40: 2-(Trimethylsilyl)ethyl 3,3-diethylpiperazine-1-carboxylate

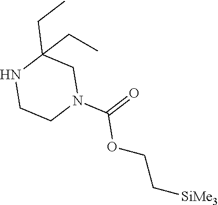 [SMILES: CCC1(CC)CN(C(=O)OCC[Si](C)(C)C)CCN1]

To a stirred solution of 2,2-diethyl-piperazine dihydrochloride (1.00 g, 4.65 mmol) in CH₂Cl₂ (20 mL) under argon atmosphere at 0° C. was added DIPEA (4.06 mL, 23.2 mmol) and 1-[2-(trimethylsilyl)ethoxycarbonyl)pyrrolidin-2,5-dione (1.20 g, 4.65 mmol). The reaction mixture was stirred for 16 h at RT. The RM was quenched with a sat. aq. NaHCO₃ solution, then extracted with CH₂Cl₂ (×2). The combined organic extracts were washed with brine, dried (Na₂SO₄), filtered and evaporated. The crude residue was purified by normal phase chromatography (eluent: MeOH in CH₂Cl₂ 0 to 10%) to give the title compound as a colorless oil. ¹H NMR (600 MHz, DMSO-d₆) δ 4.08 (t, 2H), 3.24 (m, 2H), 3.08 (s, 2H), 2.62 (m, 2H), 1.85 (m, 1H), 1.37-1.18 (m, 4H), 0.93 (t, 2H), 0.74 (t, 6H), 0.02 (s, 9H).

### Intermediate A41: 2-(Trimethylsilyl)ethyl 6,9-diazaspiro[4.5]decane-9-carboxylate

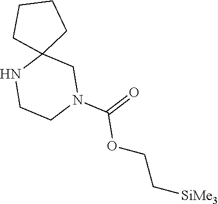 [SMILES: C[Si](C)(C)CCOC(=O)N1CCNC2(CCCC2)C1]

The title compound was prepared by a method similar to that of 2-(trimethylsilyl)ethyl 3,3-diethylpiperazine-1-carboxylate (Intermediate A40) using 6,9-diazaspiro[4.5]decane instead of 2,2-diethyl-piperazine dihydrochloride. ¹H NMR (600 MHz, DMSO-d₆) δ 4.08 (t, 2H), 3.25 (m, 2H), 3.12 (s, 2H), 2.63 (m, 2H), 2.15 (m, 1H), 1.65-1.60 (m, 2H), 1.53-1.44 (m, 4H), 1.42-1.36 (m, 2H), 0.92 (t, 2H), 0.02 (s, 9H).

### Intermediate A42: 2-(Trimethylsilyl)ethyl (4aS*,7aS*)-hexahydrofuro[3,4-b]pyrazine-1(2H)-carboxylate

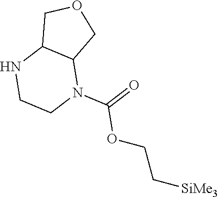 [SMILES: C[Si](C)(C)CCOC(=O)N1CCNC2COCC21]

The title compound was prepared by a method similar to that of 2-(trimethylsilyl)ethyl 3,3-diethylpiperazine-1-carboxylate (Intermediate A40) using (4aS*,7aS*)-octahydrofuro[3,4-b]pyrazine (Princeton Biomolecular Research) instead of 2,2-diethyl-piperazine dihydrochloride. MS-1: MS m/z [M+H]⁺ 273.2.

### Intermediate A43: 2-(Trimethylsilyl)ethyl 3,3-dimethylpiperazine-1-carboxylate

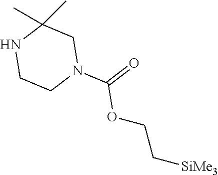 [SMILES: CC1(C)CN(C(=O)OCC[Si](C)(C)C)CCN1]

The title compound was prepared by a method similar to that of 2-(trimethylsilyl)ethyl 3,3-diethylpiperazine-1-carboxylate (Intermediate A40) using 2,2-dimethyl-piperazine dihydrochloride. ¹H NMR (600 MHz, DMSO-d₆) δ 4.08 (t, 2H), 3.23 (m, 2H), 3.05 (s, 2H), 2.67 (m, 2H), 1.89 (br. s, 1H), 0.97 (s, 6H), 0.93 (t, 2H), 0.02 (s, 9H).

### Intermediate A44: 9-Benzyl-2-oxa-6,9-diazaspiro[4.5]decane

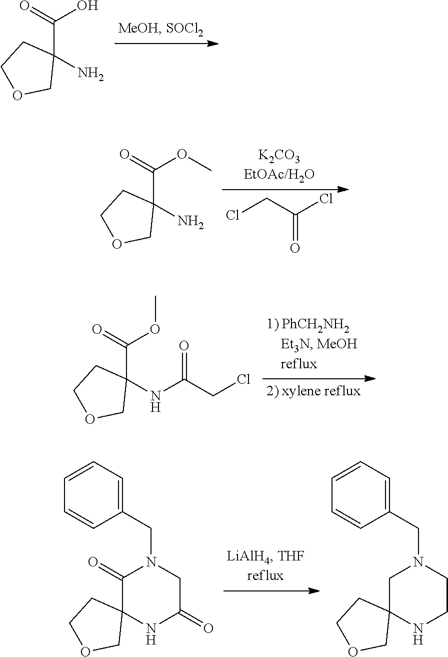

### Step 1: Methyl 3-aminotetrahydrofuran-3-carboxylate

To an ice-cooled suspension of 3-aminotetrahydro-furan-3-carboxylic acid (4.75 g, 36.2 mmol) in MeOH (47.5 mL) under nitrogen atmosphere was added dropwise thionyl chloride (7.93 mL, 109 mmol) (EXOTHERMIC!) and the reaction mixture was stirred at RT for 16 h. The RM was concentrated under vacuum, co-evaporated with toluene (×2) and dried under high vacuum to give the title compound as an hydrochloride salt (beige solid). MS-1: MS m/z [M+H]⁺ 146.1.

### Step 2: Methyl 3-(2-chloroacetamido)tetrahydrofuran-3-carboxylate

To an ice-cooled, vigorously stirred biphasic solution of methyl 3-aminotetrahydrofuran-3-carboxylate (Step 1, 6.80 g, 36.1 mmol) in EtOAc (45 mL) and water (30 mL) was added potassium carbonate (15.0 g, 108 mmol), followed by 2-chloroacetyl chloride (3.74 mL, 47.0 mmol). The reaction mixture was stirred at RT for 17 h. The reaction mixture was poured into 10% citric acid, the layers were separated and the aqueous layer was back extracted with EtOAc. The combined organic extracts were washed with a sat. aq. NaHCO₃ solution, dried (phase separator) and evaporated to give the title compound (beige solid). MS-1: MS m/z [M+H]⁺ 222.1/224.1.

### Step 3: 9-Benzyl-2-oxa-6,9-diazaspiro[4.5]decane-7,10-dione

A solution of methyl 3-(2-chloroacetamido)tetrahydrofuran-3-carboxylate (Step 2, 4.90 g, 21.5 mmol), benzylamine (7.03 mL, 64.3 mmol) and Et₃N (5.95 mL, 42.9 mmol) in MeOH (22 mL) was heated at reflux for 2.5 h. The reaction mixture was concentrated under reduced pressure to give methyl 3-(2-(benzylamino)acetamido)tetrahydrofuran-3-carboxylate which was solubilized in xylenes (22 mL) and stirred at reflux for 17 h. The reaction mixture was poured into a sat. aq. NaHCO₃ solution and extracted with CH₂Cl₂ (×2). The combined organic extracts were dried (phase separator), concentrated under reduced pressure and the crude product was purified by normal phase chromatography (eluent: MeOH in CH₂Cl₂ 0 to 5%) to give the title compound. UPLC-MS-2a: Rt=0.64 min; MS m/z [M+H]⁺ 261.2.

### Step 4: 9-Benzyl-2-oxa-6,9-diazaspiro[4.5]decane

To an ice-cooled suspension of 9-benzyl-2-oxa-6,9-diazaspiro[4.5]decane-7,10-dione (Step 3, 4.37 g, 16.8 mmol) in THF (22 mL) was slowly added LiAlH₄ (1M in THF, 33.6 mL, 33.6 mmol) and the reaction mixture was stirred at reflux for 1 h. After completion of the reaction, the RM was cooled to 0-5° C. and water (1.28 mL) was carefully added, followed by addition of NaOH (15% aq., 1.28 mL) and then water (3.83 mL). The white suspension was stirred at 0-5° C. for 30 min and Na₂SO₄ was added. The mixture was filtered and washed with EtOAc. The filtrate was concentrated under reduced pressure and the crude residue was purified by normal phase chromatography (eluent: MeOH in CH₂Cl₂ 0 to 20%) to give the title compound as a yellow oil. UPLC-MS-3: Rt=0.32 min; MS m/z [M+H]⁺ 233.3.

### Intermediate A45: 3,3-Dimethyl-1-(2,2,2-trifluoroethyl)piperazine

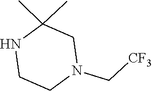

### Step 1: Tert-butyl 2,2-dimethyl-4-(2,2,2-trifluoroethyl)piperazine-1-carboxylate

To 1-Boc-2,2-dimethylpiperazine (1.00 g, 4.67 mmol), 2,2,2-trifluoroethyl trifluoromethanesulfonate (1.62 g, 7.00 mmol) and Cs₂CO₃ (3.04 g, 9.33 mmol) was added acetonitrile (23 mL) and the resulting suspension was stirred at RT under N₂ atmosphere for 64 h. The reaction mixture was filtered through a pad of celite and the filtrate was concentrated. The crude residue was purified by normal phase chromatography (eluent: MeOH in CH₂Cl₂ from 0 to 10%) to give the title compound as a clear oil. MS-1; MS m/z [M+H]⁺ 297.2.

### Step 2: 3,3-Dimethyl-1-(2,2,2-trifluoroethyl)piperazine

To a solution of tert-butyl 2,2-dimethyl-4-(2,2,2-trifluoroethyl)piperazine-1-carboxylate (1.20 g, 4.05 mmol) in 1,4-dioxane (4.1 mL) was added HCl (4N in dioxane, 12.1 mL, 48.6 mmol) and the reaction mixture was stirred at RT for 1.5 h. After completion of the reaction, the mixture was frozen and lyophilized to give the title compound as a hydrochloride salt as a white solid. MS-1; MS m/z [M+H]⁺ 197.1.

### Intermediate A46: (1R,4R)-2-(Methylsulfonyl)-2,5-diazabicyclo[2.2.1]heptane

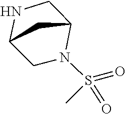

### Step 1: Tert-butyl (1R,4R)-5-(methylsulfonyl)-2,5-diazabicyclo[2.2.1]heptane-2-carboxylate

To a solution of tert-butyl (1R,4R)-2,5-diazabicyclo[2.2.1]heptane-2-carboxylate (1.50 g, 7.57 mmol) in CH₂Cl₂ (15 mL) were added triethylamine (2.11 mL, 15.1 mmol) and methanesulfonyl chloride (0.59 mL, 7.57 mmol) at 0° C. The mixture was stirred for 1 h at RT. The mixture was washed with HCl (1N in H₂O, 15 mL), sat. aq. NaHCO₃ solution (15 mL), twice with water (15 mL) and brine (15 mL), dried (MgSO₄), filtered and concentrated under vacuum to give the title compound as a white solid. UPLC-MS-4: Rt=0.57 min; MS m/z [M+H-Boc]⁺177.1.

### Step 2: (1R,4R)-2-(Methylsulfonyl)-2,5-diazabicyclo[2.2.1]heptane

To a stirred solution of tert-butyl (1R,4R)-5-(methylsulfonyl)-2,5-diazabicyclo[2.2.1]heptane-2-carboxylate (Step 1, 2.10 g, 7.60 mmol) in CH₂Cl₂ (15 mL) was added TFA (1.76 mL, 22.8 mmol) at RT and the reaction mixture was stirred at RT for 24 h. The RM was evaporated to dryness and the crude residue was dissolved in methanol (15 mL), MP-Carbonate (10 g, 7.60 mmol) was added and the mixture was swirled at 40° C. for 1 h. The mixture was filtered and the filtrate was concentrated under vacuum to give the title compound. UPLC-MS-4: Rt=0.13 min; MS m/z [M+H]⁺ 177.1.

### Intermediate A47: 4,4-Diethoxy-2,2-dimethylpiperidine

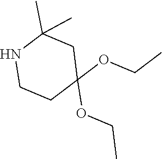

### Step 1: 2,2-Dimethylpiperidin-4-one

To a solution of tert-butyl 2,2-dimethyl-4-oxopiperidine-1-carboxylate (44.6 g, 186 mmol) in CH₂Cl₂ (220 mL) at 0° C. was added HCl (4M in 1,4-dioxane, 195 mL, 781 mmol). The reaction mixture was allowed to reach RT and stirredt at RT for 3 h. The reaction mixture was evaporated to dryness and dried under vacuum overnight to give the tithe compound as a beige solid whcih was used without purification in the next step. MS-1: MS m/z [M+H]⁺ 128.1.

### Step 2: 4,4-Diethoxy-2,2-dimethylpiperidine

To a solution of 2,2-dimethylpiperidin-4-one (34.3 g, 199 mmol) in ethanol (350 mL) cooled at 0° C. were added triethyl orthoformate (33.2 mL, 199 mmol) and PTSA (3.79 g, 19.9 mmol). The reaction mixture was slowly allowed to return to RT and was stirred overnight at RT. The reaction mixture was diluted with AcOEt and washed with a sat. aq. Na₂CO₃ solution (×2) then with water and brine. The combined aqueous layers were extracted again with AcOEt. The combined organic extracts were dried (Na₂SO₄), filtered and concentrated. ¹H NMR (400 MHz, DMSO-d₆) δ 3.36 (m, 4H), 2.67 (m, 2H), 1.52 (m, 2H), 1.45 (s, 2H), 1.07 (t, 6H), 1.03 (s, 6H).

### Intermediate A48: 8,8-Diethoxy-5-azaspiro[3.5]nonane

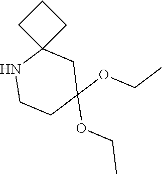

The title compound was prepared by a method similar to that of 4,4-diethoxy-2,2-dimethylpiperidine (Intermediate A47) using tert-butyl 8-oxo-5-azaspiro[3.5]nonane-5-carboxylate instead of tert-butyl 2,2-dimethyl-4-oxopiperidine-1-carboxylate. ¹H NMR (400 MHz, DMSO-d₆) δ 3.36 (q, 4H), 2.57 (m, 2H), 1.94 (m, 2H), 1.69 (m, 4H), 1.62 (s, 2H), 1.52 (m, 2H), 1.07 (t, 6H).

### Method-A49 for the preparation of A49: 4-(3-Methoxyazetidin-1-yl)-2,2-dimethylpiperidine

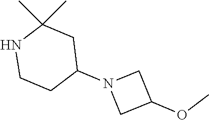

### Step 1: Tert-butyl 4-(3-methoxyazetidin-1-yl)-2,2-dimethylpiperidine-1-carboxylate

To a solution of tert-butyl 2,2-dimethyl-4-oxopiperidine-1-carboxylate (1.00 g, 4.18 mmol) in methanol (10 mL) was added 3-methoxyazetidine (0.77 g, 6.27 mmol) and acetic acid (0.24 mL, 4.18 mmol). The mixture was stirred at RT 1 h and sodium triacetoxyborohydride (2.66 g, 12.5 mmol) was added. The reaction mixture was stirred at RT for 16 h. Water was added and the mixture was extracted EtOAc (×2). The combined organic extracts were washed with water. The combined water layers were extracted with nBuOH, dried (Na₂SO₄), filtered and concentrated to give the title compound which was used without purification in the next step. UPLC-MS-2a: Rt=0.66 min; MS m/z [M+H]⁺ 300.4.

### Step 2: 4-(3-Methoxyazetidin-1-yl)-2,2-dimethylpiperidine

To a stirred solution of tert-butyl 4-(3-methoxyazetidin-1-yl)-2,2-dimethylpiperidine-1-carboxylate (Step 1, 700 mg, 2.35 mmol) in dioxane (4 mL) was added HCL (4M in dioxane, 5.86 mL, 23.5 mmol) and the reaction mixture was stirred at RT for 16 h. The reaction mixture was evaporated to dryness to give the title compound which was used without purification in the next step. UPLC-MS-2a: Rt=0.30 min; MS m/z [M+H]⁺ 199.3.

The following examples A50 to A52 were prepared using analogous methods to method-A49 from commercially available precursors (in Step 1).

| | | Method and | |
|---|---|---|---|
| Intermediate | Structure | precursors | Characterizing data |
| A50 | 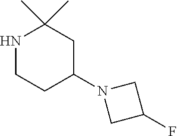 | from tert-butyl 2,2- dimethyl-4- oxopiperidine-1- carboxylate and 3- fluoroazetidine | UPLC-MS-2a: Rt = 0.62 min; MS m/z [M + H]⁺ 187.3. |
| | 4-(3-fluoroazetidin-1- | | |
| | yl)-2,2- | | |
| | dimethylpiperidine | | |
| A51 | 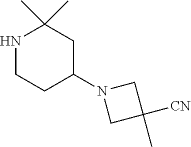 | from tert-butyl 2,2- dimethyl-4- oxopiperidine-1- carboxylate and 3- methylazetidine-3- carboxylate | UPLC-MS-2a: Rt = 0.22 min; MS m/z [M + H]⁺ 208.4. |
| | 1-(2,2- | | |
| | dimethylpiperidin-4-yl)- | | |
| | 3-methylazetidine-3- | | |
| | carbonitrile | | |
| A52 | 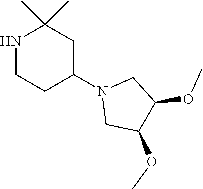 [SMILES: CO[C@H]1CN(C2CCNC(C)(C)C2)C[C@H]1OC] | from tert-butyl 2,2- dimethyl-4- oxopiperidine-1- carboxylate and cis- 3,4- dimethoxypyrrolidine | UPLC-MS-4: Rt = 0.14 min; MS m/z [M + H]⁺ 243.3. |
| | 4-((3S,4R)-3,4- | | |
| | dimethoxypyrrolidin-1- | | |
| | yl)-2,2- | | |
| | dimethylpiperidine | | |

### Intermediate A53: 4-(2,2-Dimethylpiperidin-4-yl)morpholine

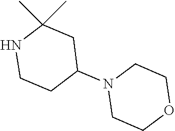

### Step 1: Tert-butyl 2,2-dimethyl-4-morpholinopiperidine-1-carboxylate

Molecular sieves (4° A) were added to a mixture of tert-butyl 2,2-dimethyl-4-oxopiperidine-1-carboxylate (5.00 g, 22 mmol) and morpholine (2.27 mL, 22.0 mmol) in DCE (150 mL) under N₂ atmosphere and the mixture was stirred for 30 min. Titanium (IV) isopropoxide (6.51 mL, 22.0 mmol) was added dropwise and the reaction mixture was heated to 60° C. for 16 h. The RM was then cooled to 0° C., sodium cyanoborohydride (1.66 g, 26.4 mmol) was added portionwise and the RM was heated again at 70° C. for 8 h. After completion of the reaction, the RM was concentrated under vacuum. The crude residue was dissolved in EtOAc and the insoluble precipitate was filtered through a pad of celite and washed with EtOAc. The filtrate was washed with a sat. aq. sol. of sodium bicarbonate, with brine, dried (Na₂SO₄), filtered and concentrated under vacuum. The crude product was purified by normal phase chromatography (eluent: 0 to 5% MeOH in CH₂Cl₂) to give the title compound. UPLC-MS-5: Rt=1.34 min, MS m/z [M+H]⁺ 299.3.

### Step 2: 4-(2,2-Dimethylpiperidin-4-yl)morpholine

To a solution of tert-butyl 2,2-dimethyl-4-morpholinopiperidine-1-carboxylate (3.82 g, 12.7 mmol) in CH₂Cl₂ (15 mL) at 0° C. was added HCl (4M in dioxane, 12.8 mL) and the reaction mixture was stirred at RT for 2 h. The RM was concentrated under vacuum and co-distilled with toluene to afford the title product as a HCl salt. The salt was dissolved in MeOH (15 mL), tetra alkylammonium carbonate polymer bound resin (Sigma Aldrich cat. 540293, 16 g) was added and the round bottom flask was swirled at 40° C. until the pH of the solution became basic. The mixture was filtered through Millipore and washed with MeOH. The filtrate was concentrated under vacuum and the crude residue was purified by normal phase chromatography on basic alumina (eluent: 0 to 5% MeOH in CH₂Cl₂) to give the title compound. UPLC-MS-5: Rt=0.28 min, MS m/z [M+H]⁺ 199.3.

### Intermediate A54: (R)—N-(2,2-Dimethylpiperidin-4-yl)-N-methylacetamide

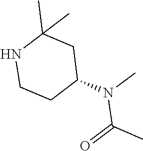

### Step 1: Tert-butyl (R)-2,2-dimethyl-4-(methylamino)piperidine-1-carboxylate

To a stirred solution of tert-butyl 2,2-dimethyl-4-oxopiperidine-1-carboxylate (19.6 g, 86 mmol) in MeOH (200 mL) was added under argon MeNH₂ (2M in MeOH, 86 mL, 172 mmol) and AcOH (4.94 mL, 86 mmol). Then, the reaction mixture was stirred for 1 h at RT and NaBH(OAc)₃ (54.8 g, 259 mmol) was added. After completion, the reaction mixture was quenched with 250 mL of H₂O and extracted with CH₂Cl₂. The aqueous layer was basified with NaOH (1N) to pH 9-10 and was extracted then extracted with CH₂Cl₂ (×2). The combined organic extracts were dried (Na₂SO₄), filtered and evaporated. The crude residue was purified by normal phase chromatography (eluent: MeOH in CH₂Cl₂ from 0% to 10%) to give the title compound as a white solid. UPLC-MS-2a: Rt=0.62 min; MS m/z [M+H]⁺ 243.3.

### Step 2: Tert-butyl-2,2-dimethyl-4-(N-methylacetamido)piperidine-1-carboxylate

To a stirred solution of tert-butyl (R)-2,2-dimethyl-4-(methylamino)piperidine-1-carboxylate (Step 1, 18.7 g, 77 mmol) in CH₂Cl₂ (250 mL) under argon were added acetyl chloride (8.23 mL, 116 mmol) and NEt₃ (26.9 mL, 193 mmol) and the reaction mixture was stirred for 30 min at 0° C. The reaction mixture was quenched with 250 mL of sat. aq. NaHCO₃, extracted with CH₂Cl₂ (×2), the combined organic extracts were washed with a sat. aq. NaHCO₃ solution, dried (Na₂SO₄), filtered and evaporated. The crude residue was purified by normal phase chromatography (eluent: MeOH in CH₂Cl₂ from 0% to 8%) to give the title compound as a yellow oil. The enantiomers were separated by chiral SFC (C-SFC-13; mobile phase: CO₂/IPA: 80/20) to give tert-butyl (R)-2,2-dimethyl-4-(N-methylacetamido)piperidine-1-carboxylate as the first eluting enantiomer; C-SFC-17 (mobile phase: CO₂/[IPA+0.05% DEA] 95/5 to 60/40) Rt=5.67 min, UPLC-MS-2a: Rt=0.6 min; MS m/z [M+H]⁺ 285.2 and tert-butyl (S)-2,2-dimethyl-4-(N-methylacetamido)piperidine-1-carboxylate as the second eluting enantiomer: C-SFC-17 (mobile phase: CO₂/[IPA+0.05% DEA] 95/5 to 60/40) Rt=5.90 min, UPLC-MS-2a: Rt=0.6 min; MS m/z [M+H]⁺ 285.2.

### Step 3: (R)—N-(2,2-Dimethylpiperidin-4-yl)-N-methylacetamide

To a stirred solution of tert-butyl (R)-2,2-dimethyl-4-(N-methylacetamido)piperidine-1-carboxylate (Step 2, 10.7 g, 37.6 mmol) in dioxane (100 mL) was added under argon HCl (4N in dioxane, 94 mL, 376 mmol) and the reaction mixture was stirred for 16 h at RT. The reaction mixture was concentrated to afford the title compound as a hydrochloride salt as white solid. UPLC-MS-2a: Rt=0.22 min; MS m/z [M+H]⁺ 185.3.

### Intermediate A55: (S)—N-(2,2-Dimethylpiperidin-4-yl)-N-methylacetamide

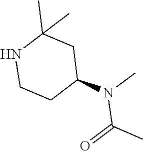

To a stirred solution of tert-butyl (S)-2,2-dimethyl-4-(N-methylacetamido)piperidine-1-carboxylate (Step 2 in the synthesis of Intermediate A54, 10.0 g, 35.2 mmol) in dioxane (100 mL) was added under argon HCl (4N in dioxane, 88 mL, 352 mmol) and the reaction mixture was stirred for 20 h at RT. The RM was concentrated to afford the title compound as a hydrochloride salt as white solid. UPLC-MS-2a: Rt=0.22 min; MS m/z [M+H]⁺ 185.2.

### Intermediate A56: 4-((Tert-butyldiphenylsilyl)oxy)-2,2-dimethylpiperidine

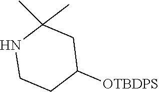

### Step 1: Tert-butyl 4-hydroxy-2,2-dimethylpiperidine-1-carboxylate

To a solution of tert-butyl 2,2-dimethyl-4-oxopiperidine-1-carboxylate (2.00 g, 8.81 mmol) in MeOH (20 mL) at 0° C. was added under nitrogen NaBH₄ (0.50 g, 13.2 mmol) portion wise over a period of 10 min and the reaction mixture was stirred at 0° C. for 40 min. The reaction mixture was quenched with cold water and extracted with EtOAc. The combined organic extracts were washed with brine, dried (Na₂SO₄), filtered and concentrated under vacuum to give the title compound which was used directly in the next step without further purification. UPLC-MS-5: Rt=1.55 min; MS m/z [M−56]⁺ 174.3.

### Step 2: Tert-butyl 4-((tert-butyldiphenylsilyl)oxy)-2,2-dimethylpiperidine-1-carboxylate

To a solution of tert-butyl 4-hydroxy-2,2-dimethylpiperidine-1-carboxylate (Step 1, 1.50 g, 6.55 mmol) in CH₂Cl₂ (20 mL) was added at 0° C. under nitrogen atmosphere imidazole (0.89 g, 13.1 mmol) and the mixture was stirred for 10 min at 0° C. Tert-butyldiphenylsilyl chloride (2.16 g, 7.86 mmol) was added slowly and the RM was stirred at RT for 16 h. The reaction mixture was quenched with cold water and extracted with CH₂Cl₂. The combined organic extracts were washed with brine, dried (Na₂SO₄), filtered and concentrated under vacuum to give the title compound which was used directly in the next step without further purification. ¹H NMR (400 MHz, CDCl₃) δ 7.74-7.66 (m, 6H), 7.46-7.37 (m, 4H), 3.91-3.89 (m, 1H), 3.72-3.66 (m, 1H), 3.14-3.07 (m, 1H), 1.81-1.80 (m, 1H), 1.68-1.62 (m, 1H), 1.59-1.56 (m, 5H) 1.54-1.53 (m, 3H), 1.50 (s, 9H), 1.15-1.01 (m, 9H).

### Step 3: 4-((Tert-butyldiphenylsilyl)oxy)-2,2-dimethylpiperidine

To a solution of tert-butyl 4-((tert-butyldiphenylsilyl)oxy)-2,2-dimethylpiperidine-1-carboxylate (Step 2, 3.00 g, 6.41 mmol) in CH₂Cl₂ (15 mL) at 0° C. under nitrogen atmosphere was added TFA (30 mL) and reaction mixture was stirred at RT for 2 h. The RM was concentrated under reduced pressure and co distilled with CH₂Cl₂ several times. The crude residue was purified by reverse phase chromatography (eluent: 0 to 45% CH₃CN in H₂O containing 0.1% NH₃) to afford the title product. UPLC-MS-5: Rt=1.76 min; MS m/z [M+H]⁺ 368.7.

### Intermediate A57: 1-Methyl-1,4,5,6-tetrahydropyrrolo[3,4-c]pyrazole

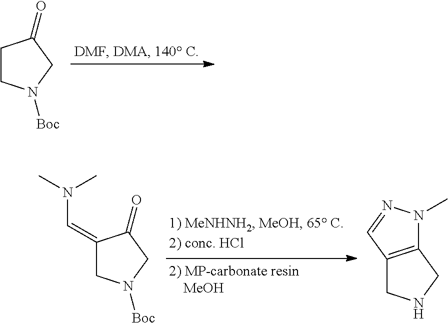

### Step 1: Tert-butyl-3-((dimethylamino)methylene)-4-oxopyrrolidine-1-carboxylate

A solution of tert-butyl 3-oxopyrrolidine-1-carboxylate (2.50 g, 13.4 mmol) in DMF-DMA (1/1, 18 mL) was stirred at 140° C. for 1 h. After completion of the reaction, the reaction mixture was concentrated to dryness, dissolved in a minimum amount of CH₂Cl₂ and triturated with hexane to afford the title product. ¹H NMR (400 MHz, DMSO-d₆) δ 7.22 (s, 1H), 4.50 (m, 2H), 3.59 (m, 2H), 3.07 (s, 6H), 1.50 (s, 9H).

### Step 2: 1-Methyl-1,4,5,6-tetrahydropyrrolo[3,4-c]pyrazole hydrochloride

To a solution of tert-butyl-3-((dimethylamino)methylene)-4-oxopyrrolidine-1-carboxylate (Step 1, 2.00 g, 8.33 mmol) in MeOH (24 mL) was added methyl hydrazine (0.47 g, 10.3 mmol) and the reaction mixture was heated under reflux for 2.5 h in a sealed tube. After completion of reaction, the RM was concentrated to dryness and the crude product was triturated with pentane and Et₂O to give a yellow solid. The solid was dissolved in TFA (40 mL) at 0° C. and the solution was stirred for 1 h at RT and then concentrated. The residue was dissolved in ethanol (10 mL) and treated with conc. aq. HCl (2.40 mL). After removing the solvent under vacuum, the resulting solid was triturated with 2-propanol and Et₂O to afford the title product as a hydrochloride salt. UPLC-MS-5=Rt=0.26 min, MS m/z [M+H]⁺ 124.15, ¹H NMR (400 MHz, DMSO-d₆) δ 10.72 (s, 1H), 7.26 (s, 1H), 4.21 (m, 2H), 4.04 (m, 2H), 3.78 (s, 3H).

### Step 3: 1-Methyl-1,4,5,6-tetrahydropyrrolo[3,4-c]pyrazole

To a solution of 1-methyl-1,4,5,6-tetrahydropyrrolo[3,4-c]pyrazole hydrochloride (0.20 g, 1.25 mmol) in MeOH (4 mL) was added tetra alkyl ammonium carbonate polymer bound (0.50 g) and the mixture was stirred for 15 min at RT. The pH of the reaction mixture changed to basic and the reaction mixture was filtered through Millipore and concentrated under vacuum to afford the title product. ¹H NMR (400 MHz, DMSO-d₆) δ 7.21 (s, 1H), 4.26 (m, 2H), 4.10 (m, 2H), 3.75 (s, 3H).

### Intermediate A58: (3aS*,7aS*)-2-Methyloctahydro-1H-pyrrolo[3,4-c]pyridin-1-one

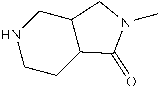

### Step 1: (3aS*,7aS*)-5-Benzyl-2-methyloctahydro-1H-pyrrolo[3,4-c]pyridin-1-one

A mixture of (3aR*,7aS*)-5-benzyloctahydro-1H-pyrrolo[3,4-c]pyridin-1-one (950 mg, 4.12 mmol) and NaH (60% in mineral oil, 330 mg, 8.25 mmol) in THF (20 mL) was stirred at 0° C. under a nitrogen atmosphere for 5 min, before addition of iodomethane (270 μL, 4.33 mmol). The reaction mixture was stirred at RT for 4 h. NaH (60% in mineral oil, 495 mg, 12.4 mmol) and iodomethane (405 μL, 6.50 mmol) were added and the RM was further stirred for 6 h. The reaction mixture was carefully poured into a sat. aq. NaHCO₃ solution and extracted with CH₂Cl₂ (×3). The combined organic extracts were dried (phase separator) and evaporated. The crude residue was purified by normal phase chromatography (eluent: MeOH in CH₂Cl₂ from 0 to to 10%) to give the title compound. NMR (600 MHz, DMSO-d₆) δ 7.33-7.23 (m, 5H), 4.41 (d, 1H), 4.38 (d, 1H), 3.32 (m, 1H), 2.84 (d, 1H), 2.72 (s, 3H), 2.66 (m, 1H), 2.45 (m, 1H), 2.40 (m, 2H), 1.83-1.69 (m, 4H).

### Step 2: (3aS*,7aS*)-2-Methyloctahydro-1H-pyrrolo[3,4-c]pyridin-1-one

In an autoclave, to a solution of (3aS*,7aS*)-5-benzyl-2-methyloctahydro-1H-pyrrolo[3,4-c]pyridin-1-one (Step 1, 705 mg, 2.89 mmol) in MeOH (40 mL) was added Pd(OH)₂/C 20% (50% wet, 0.10 g). The mixture was placed under a hydrogen atmosphere (4 bars) and stirred for 14 h. Pd(OH)₂/C 20% (50% wet, 0.11 g) was added again and the mixture further stirred under hydrogen atmosphere for 15 h. Pd/C 10% (50% wet, 0.05 g) was added and the mixture further stirred under hydrogen atmosphere for 14 h. The reaction mixture was filtered over a pad of celite and washed with MeOH and CH₂Cl₂. The filtrate was evaporated to give the title product which was used without purification in the next step. NMR (600 MHz, DMSO-d₆) δ 3.29 (m, 1H), 2.87 (m, 1H), 2.84 (m, 1H), 2.72 (s, 3H), 2.64 (m, 1H), 2.41 (m, 1H), 2.28-2.20 (m, 2H), 1.78 (m, 1H), 1.70 (m, 1H), 1.57 (m, 1H).

### Intermediate A59: (3aR*,7aR*)-1-methyloctahydro-2H-pyrrolo[3,2-c]pyridin-2-one

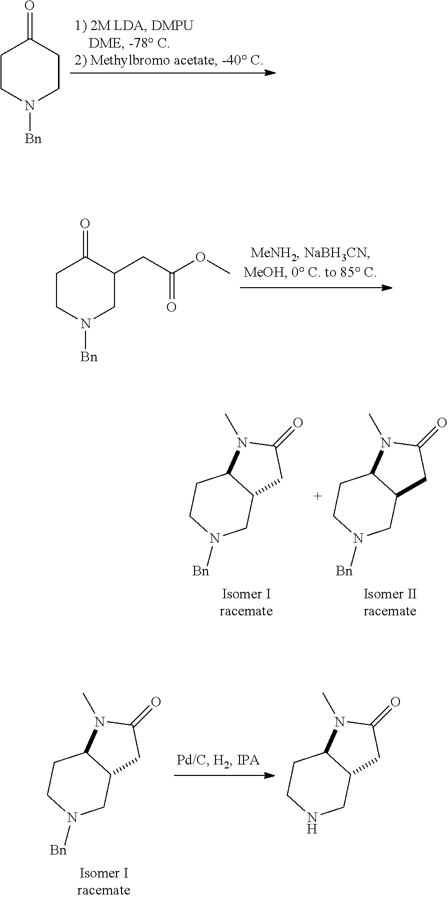

### Step 1: Methyl 2-(1-benzyl-4-oxopiperidin-3-yl)acetate

Under nitrogen atmosphere, to anhydrous DME (350 mL) were successively added LDA (2M in THF, 34.3 mL, 68.6 mmol) at −78° C. and a solution of N-benzyl piperidine-4-one (10.0 g, 52.8 mmol) in anhydrous DME (10 mL). The mixture was stirred for 5 min and 1,3-dimethyl-3,4,5,6-tetrahydro-2(1H)-pyrimidinone (10.0 g, 52.8 mmol) was added. The mixture was stirred at −78° C. for 20 min and methylbromo acetate (10.5 g, 68.6 mmol) was added over a period of 7 min. The reaction mixture was stirred for 20 min at −78° C. and was allowed to gradually warm to −40° C. and was further stirred for 45 min. After completion of reaction, the reaction mixture was quenched by adding a sat. aq. NaHCO₃ solution at −40° C. and was extracted with EtOAc (×2). The combined organic layer was washed with brine, dried (Na₂SO₄), filtered and concentrated under vacuum. The crude residue was purified by normal phase chromatography (eluent: 0 to 5% MeOH in CH₂Cl₂) to give the title product. UPLC-MS-5: Rt=1.34 min, MS [M+H]⁺ 262.2.

### Step 2: (3aR*,7aR*)-5-Benzyl-1-methyloctahydro-2H-pyrrolo[3,2-c]pyridin-2-one isomer-I and (3aS*,7aR*)-5-benzyl-1-methyloctahydro-2H-pyrrolo[3,2-c]pyridin-2-one isomer-II

To a solution of methyl 2-(1-benzyl-4-oxopiperidin-3-yl)acetate (Step 1, 5.33 g, 20.4 mmol) in MeOH (150 mL) at 0° C. were added methylamine hydrochloride (20.7 g, 306.3 mmol) and sodium cyanoborohydride (2.56 g, 40.9 mmol) and the mixture was heated in a screw capped vial at 85° C. for 4 days. After completion of the reaction, the reaction mixture was basified by addition of a sat. aq. NaHCO₃ solution (pH 8) and extracted with EtOAc (×3). The combined organic layer was washed with brine, dried (Na₂SO₄), filtered and concentrated under vacuum. The crude residue was purified by normal phase chromatography (eluent: MeOH:hexane:ethyl acetate, 0.7:3:6.3) to give the first eluting peak of the title product: Isomer I: UPLC-MS-10: Rt=4.91 min, MS m/z [M+H]⁺ 245.0; ¹H NMR (400 MHz, Methanol-d₄) δ 7.36-7.27 (m, 5H), 3.69-3.61 (m, 2H), 3.15 (m, 1H), 3.08 (m, 1H), 3.00-2.97 (m, 1H), 2.76 (s, 3H), 2.32-2.25 (m, 2H), 2.20-2.0 (m, 4H), 1.68-1.60 (m, 1H), and the second eluting peak of the title product: Isomer II: UPLC-MS-5: Rt=1.19 min, MS m/z [M+H]⁺ 245.0; ¹H NMR (400 MHz, Methanol-d₄) δ 7.35-7.27 (m, 5H), 3.65-3.61 (m, 2H), 3.56-3.48 (m, 2H), 2.80 (s, 3H), 2.68-2.59 (m, 1H), 2.58-2.37 (m, 3H), 2.27-2.20 (m, 1H), 2.18-2.09 (m, 1H), 2.08-1.98 (m, 1H), 1.96-1.87 (m, 1H).

### Step 3: (3aR*,7aR*)-1-Methyloctahydro-2H-pyrrolo[3,2-c]pyridin-2-one

A solution of (3aR*,7aR*)-5-benzyl-1-methyloctahydro-2H-pyrrolo[3,2-c]pyridin-2-one isomer-I (Step 2 Isomer I, 2.10 g, 8.60 mmol) in isopropyl alcohol (20 mL) was purged with nitrogen gas for 10 min under stirring, Pd/C 10% (1.30 g, 1.2 mmol) was added and the reaction mixture was stirred under the 1 atmosphere of hydrogen at RT for 20 h. After completion of the reaction, the reaction mixture was filtered through a pad of celite and washed with isopropyl alcohol. The filtrate was concentrated under vacuum to afford the title product, which was used in the next step without further purification. UPLC-MS-11: Rt=3.60 min, MS m/z [M+H]⁺ 155.0.

### Intermediate A60: 2,7,7-Trimethyl-2,8-diazaspiro[4.5]decan-3-one

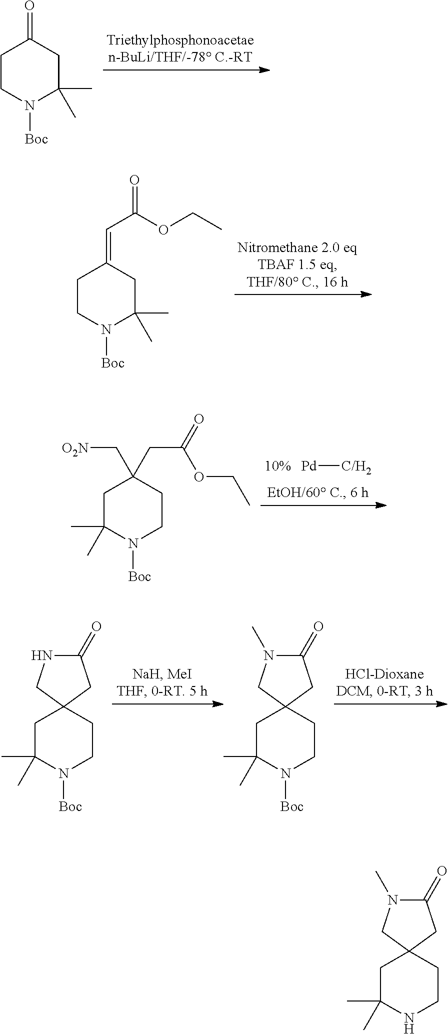

### Step 1: Tert-butyl-4-(2-ethoxy-2-oxoethylidene)-2,2-dimethylpiperidine-1-carboxylate

Triethylphosphonoacetate (5.90 g, 26.4 mmol) was dissolved in dry THF (40 mL) and cooled to −78° C. under nitrogen atmosphere. n-BuLi (3.60 M in hexane, 7.00 mL, 26.4 mmol) was added and the mixture was stirred for 30 min at −78° C. Tert-butyl 2,2-dimethyl-4-oxopiperidine-1-carboxylate (2.00 g, 8.80 mmol) was then added at −78° C. and the reaction mixture was stirred at RT for 16 h. After completion of the reaction, the RM was diluted with water and extracted with EtOAc (×3). The combined organic extracts were washed with brine, dried (Na₂SO₄), filtered and concentrated under reduced pressure. The crude residue was purified by normal phase chromatography (eluent: 5% EtOAc in hexane) to give the title compound. UPLC-MS-5: Rt=2.03 min, MS m/z [M-Boc]⁺198.4.

### Step 2: Tert-butyl 4-(2-ethoxy-2-oxoethyl)-2,2-dimethyl-4-(nitromethyl)piperidine-1-carboxylate

To a stirred solution of tert-butyl-4-(2-ethoxy-2-oxoethylidene)-2,2-dimethylpiperidine-1-carboxylate (Step 1, 2.78 g, 9.36 mmol) in THF (30 mL) under argon atmosphere, TBAF (4.43 g, 14.0 mmol) was added and the mixture was stirred at RT for 5 min. Nitromethane (1.14 g, 18.7 mmol) was then added and the reaction mixture was heated at 80° C. for 16 h. The reaction mixture was diluted with water and extracted with EtOAc (×2). The combined organic later was washed with brine, dried (Na₂SO₄), filtered and concentrated under reduced pressure. The crude residue was purified by normal phase chromatography (eluent: 5% EtOAc in hexane) to give the title product. UPLC-MS-5: Rt=2.01 min, MS m/z [M+H]⁺ 359.35.

### Step 3: Tert-butyl 7,7-dimethyl-3-oxo-2,8-diazaspiro[4.5]decane-8-carboxylate

To a solution of tert-butyl 4-(2-ethoxy-2-oxoethyl)-2,2-dimethyl-4-(nitromethyl)piperidine-1-carboxylate (Step 2, 2.80 g, 7.82 mmol) in EtOH (30 mL) was added Pd/C 10% (1.40 g) and the reaction mixture was stirred under hydrogen pressure (5 atm.) at 60° C. for 6 h. After completion of the reaction, the reaction mixture was filtered through a pad of celite and washed with EtOAc. The filtrate was concentrated under reduced pressure to give the title compound which was used in the next step without further purification. UPLC-MS-5: Rt=1.57 min, MS m/z [M+H]⁺ 283.3.

### Step 4: Tert-butyl 2,7,7-trimethyl-3-oxo-2,8-diazaspiro[4.5]decane-8-carboxylate

To a solution of tert-butyl 7,7-dimethyl-3-oxo-2,8-diazaspiro[4.5]decane-8-carboxylate (Step 3, 2.30 g, 7.90 mmol) in dry THF (20 mL) at 0° C. under nitrogen atmosphere was added sodium hydride (55% in oil, 0.69 g, 15.8 mmol) and the mixture was stirred at RT for 30 min. Methyl iodide (5.60 g, 39.5 mmol, 5.0 equiv) was then added slowly at 0° C. and the reaction mixture was allowed to reach RT and stirred for 5 h. The RM was diluted with water and extracted with EtOAc (×2). The combined organic layer was washed with brine, dried (Na₂SO₄), filtered and concentrated under reduced pressure. The crude residue was purified by normal phase chromatography (eluent: 0 to 2% MeOH in CH₂Cl₂) to give the title compound. UPLC-MS-5: Rt=1.58 min, MS m/z [M+H]⁺ 297.6.

### Step 5: 2,7,7-Trimethyl-2,8-diazaspiro[4.5]decan-3-one

To a solution of tert-butyl 2,7,7-trimethyl-3-oxo-2,8-diazaspiro[4.5]decane-8-carboxylate (Step 4, 1.50 g, 5.06 mmol) in CH₂Cl₂ (15 mL) at 0° C. under nitrogen atmosphere was added dropwise HCl (4M in dioxane, 8.00 mL, 32 mmol) and the reaction mixture was stirred at RT for 3 h. The RM was concentrated under reduced pressure and co-distilled with CH₂Cl₂ several times to give a crude residue which was dissolved in MeOH (10 mL). Tetra alkylammonium carbonate resin bound (Sigma Aldrich cat. 540293, 2.50 g) was added and the mixture was swirled at 40° C. for 2 h, then was filtered through a pad of celite and the filtrate was concentrated to give the title compound. UPLC-MS-5: Rt=0.30 min, MS m/z [M+H]⁺ 197.2.

### Intermediate A61: 8,8-Dimethyl-5,6,7,8-tetrahydroimidazo[1,2-a]pyrazine

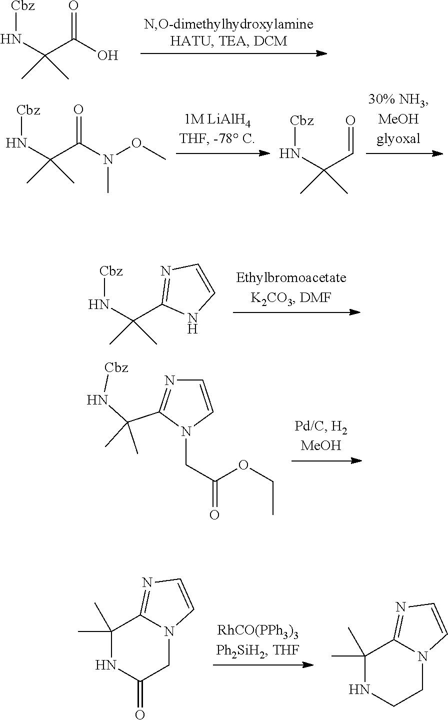

### Step 1: Benzyl (1-(methoxy(methyl)amino)-2-methyl-1-oxopropan-2-yl)carbamate

Z-2-Methylalanine (20.0 g, 84.0 mmol) was dissolved in CH₂Cl₂ (400 mL) and cooled to 0° C. under nitrogen atmosphere. N,O-dimethylhydroxyl amine (9.60 g, 101 mmol), HATU (48.0 g, 127 mmol) and Et₃N (46 mL, 337 mmol) were added and the reaction mixture was stirred at RT for 16 h. After completion of the reaction, the RM was quenched with water and extracted with EtOAc. The combined organic layer was washed with water, brine, dried (Na₂SO₄), filtered and concentrated under vacuum. The crude residue was purified normal phase chromatography (eluent: 10 to 50% EtOAc in hexane) to give the title product. UPLC-MS-5: Rt=1.51 min, MS m/z [M+H]⁺ 281.6.

### Step 2: Benzyl (2-methyl-1-oxopropan-2-yl)carbamate

Benzyl (1-(methoxy(methyl)amino)-2-methyl-1-oxopropan-2-yl)carbamate (Step 1, 13.0 g, 46.4 mmol) was dissolved in THF (260 mL) and cooled to −78° C. under nitrogen atmosphere. LiAlH₄ (1M in THF, 46.4 mL, 46.4 mmol) was added dropwise and the reaction mixture was stirred at −78° C. for 40 min. After completion of the reaction, the RM was quenched by dropwise addition of EtOAc at −78° C. A saturated solution of sodium sulphate was then added very slowly and the mixture was stirred at RT for 15 min. The mixture was filtered through a pad of celite, washed with EtOAc and the filtrate was concentrated under vacuum to afford the title product which was used in the next step without further purification. ¹H NMR (400 MHz, CDCl₃) δ 9.47 (s, 1H), 7.43-7.36 (m, 5H), 5.29 (s, 1H), 5.13 (s, 2H), 1.42 (s, 6H).

### Step 3: Benzyl (2-(1H-imidazol-2-yl)propan-2-yl)carbamate

To a solution of benzyl (2-methyl-1-oxopropan-2-yl)carbamate (Step 2, 10.5 g, 47.7 mmol) in MeOH (100 mL) cooled to 0° C. was added NH₃ (30% aq. solution, 100 mL) followed by glyoxal (40% in water, 55.4 g, 955 mmol) and the reaction mixture was stirred at RT for 48 h. After completion of the reaction, the RM was concentrated under vacuum, diluted with water and extracted with EtOAc. The combined organic layer was washed with brine, dried (Na₂SO₄), filtered and concentrated under vacuum. The crude residue was purified by normal phase chromatography (eluent: 0 to 60% EtOAc in hexane) to give the title product. ¹H NMR (400 MHz, DMSO-d₆) δ 11.67 (s, 1H), 7.48 (s, 1H), 7.36-7.32 (m, 5H), 6.80 (s, 1H), 4.96 (s, 2H), 1.55 (s, 6H). UPLC-MS-5: Rt=1.28 min, MS m/z [M+H]⁺ 260.6.

### Step 4: Ethyl 2-(2-(2-(((benzyloxy)carbonyl)amino)propan-2-yl)-1H-imidazol-1-yl)acetate

To a solution of benzyl (2-(1H-imidazol-2-yl)propan-2-yl)carbamate (Step 3, 5.00 g, 19.3 mmol) dissolved in DMF (100 mL) were added K₂CO₃ (5.32 g, 38.6 mmol) and ethylbromoacetate (4.80 g, 29.0 mmol) and the reaction mixture was stirred at RT for 15 h. After completion of the reaction, the RM was quenched with water and extracted with EtOAc. The combined organic layer was washed with cold water, brine, dried (Na₂SO₄), filtered and concentrated under vacuum to afford the title product. UPLC-MS-5: Rt=1.34 min, MS m/z [M+H]⁺ 346.6.

### Step 5: 8,8-Dimethyl-7,8-dihydroimidazo[1,2-a]pyrazin-6(5H)-one

To a solution of ethyl 2-(2-(2-(((benzyloxy)carbonyl)amino)propan-2-yl)-1H-imidazol-1-yl)acetate (Step 4, 7.00 g, 20.3 mmol) in MeOH (250 mL) was added Pd/C 10% (2.40 g, 2.20 mmol) and the reaction mixture was stirred at RT under 1 atmosphere of hydrogen pressure for 15 h. After completion of the reaction, the RM was filtered through a pad of celite and washed with MeOH. The filtrate was concentrated under vacuum and the crude residue was purified by normal phase chromatography on basic alumina (eluent: 0 to 5% MeOH in CH₂Cl₂) to obtain the title product. 1H NMR (400 MHz, DMSO-d₆) δ 8.62 (s, 1H), 7.05 (s, 1H), 6.90 (s, 1H), 4.67 (s, 2H), 1.50 (s, 6H).

### Step 6: 8,8-Dimethyl-5,6,7,8-tetrahydroimidazo[1,2-a]pyrazine

To a solution of 8,8-dimethyl-7,8-dihydroimidazo[1,2-a]pyrazin-6(5H)-one (Step 5, 1.00 g, 6.06 mmol) in dry THF (6 mL) was added diphenylsilane (10 mL) and the reaction mixture was degassed with nitrogen for 10 min. RhCO(PPh₃)₃ (0.28 g, 0.30 mmol) was added and the reaction mixture was stirred at RT for 16 h. After completion of the reaction, the RM was diluted with EtOAc and extracted with cold 1N HCl aq. solution. The aqueous layer was then basified with a sat. aq. NaHCO₃ solution and extracted with EtOAc (multiple times). The combined organic layer was concentrated under vacuum and the crude residue was purified by reverse phase chromatography (eluent: 0 to 30% CH₃CN in water containing 0.1% NH₃) to give the title product. 1H NMR (400 MHz, DMSO-d₆) δ 6.92 (s, 1H), 6.70 (s, 1H), 4.60 (m, 2H), 3.07 (m, 2H), 2.24 (m 1H), 1.34 (s, 6H).

### Intermediate A62a and A62b: 4-Methyl-4,5,6,7-tetrahydropyrazolo[1,5-a]pyrazine

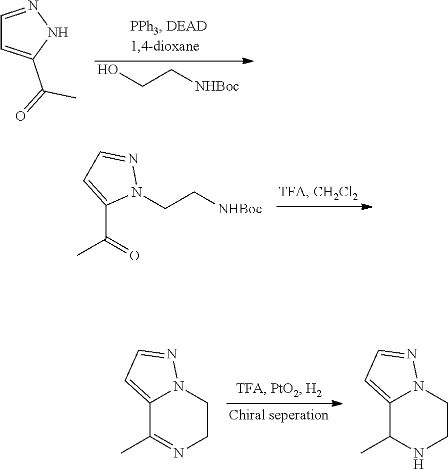

### Step 1: Tert-butyl (2-(5-acetyl-1H-pyrazol-1-yl)ethyl)carbamate

Under argon atmosphere, to a solution of 1-(1H-pyrazol-5-yl)ethan-1-one (10.0 g, 91.0 mmol) and tert-butyl (2-hydroxyethyl)carbamate (18.3 mL, 118 mmol) in 1,4-dioxane (100 mL) were added PPh₃ (35.7 g, 136 mmol) and DEAD (21.6 mL, 136 mmol) at 0° C. and the reaction mixture was stirred at RT for 16 h. The reaction mixture was quenched with a sat. aq. NaHCO₃ solution and extracted with EtOAc (×2). The combined organic layers were dried (Na₂SO₄), filtered and concentrated in vacuo and the crude residue was purified by normal phase chromatography (eluent: EtOAc in c-hexane 25 to 50%) to give the title compound as a colorless oil. UPLC-MS-2b: Rt=0.84 min; MS m/z [M+H]⁺ 254.2.

### Step 2: 4-Methyl-6,7-dihydropyrazolo[1,5-a]pyrazine

To a solution of tert-butyl (2-(5-acetyl-1H-pyrazol-1-yl)ethyl)carbamate (Step 1, 12.8 g, 50.5 mmol) in CH₂Cl₂ (200 mL) was added TFA (19.5 mL, 253 mmol) and the reaction mixture was stirred at RT for 2 h. The reaction mixture was quenched with a sat. aq. NaHCO₃ solution and extracted with CH₂Cl₂ (×2). The combined organic layers were dried (Na₂SO₄), filtered and concentrated in vacuo to give the title compound as a colorless oil. UPLC-MS-2b: Rt=0.24 min; MS m/z [M+H]⁺ 136.0.

### Step 3: 4-Methyl-4,5,6,7-tetrahydropyrazolo[1,5-a]pyrazine

To a solution of 4-methyl-6,7-dihydropyrazolo[1,5-a]pyrazine (Step 2, 3.00 g, 22.2 mmol) in THF (120 mL) was added PtO₂ (504 mg, 2.22 mmol) and the reaction mixture was stirred at RT under H₂ atmosphere for 2 h. The reaction mixture was filtered through a celite pad and the filtrate was concentrated in vacuo. The crude residue was purified by normal phase chromatography (eluent: MeOH in CH₂Cl₂ 0 to 10%) to give the title compound as a colorless oil. The enantiomers were separated by chiral SFC (C-SFC-25; mobile phase: CO₂/MeOH: 90/10) to give 4-methyl-4,5,6,7-tetrahydropyrazolo[1,5-a]pyrazine Intermediate A62a as the first eluting enantiomer: ¹H NMR (400 MHz, DMSO-d₆) δ 7.33 (d, 1H), 6.01 (dd, 1H), 3.98 (ddd, 1H), 3.94-3.85 (m, 2H), 3.23 (dd, 1H), 2.99 (m, 1H), 2.59 (br s, 1H), 1.31 (d, 3H) and 4-methyl-4,5,6,7-tetrahydropyrazolo[1,5-a]pyrazine Intermediate A62b as the second eluting enantiomer: ¹H NMR (400 MHz, DMSO-d₆) δ 7.33 (d, 1H), 6.01 (dd, 1H), 3.98 (ddd, 1H), 3.94-3.85 (m, 2H), 3.23 (dd, 1H), 2.99 (m, 1H), 2.59 (br s, 1H), 1.31 (d, 3H).

### Intermediate A63: 4,4-Dimethyl-4,5,6,7-tetrahydropyrazolo[1,5-a]pyrazine

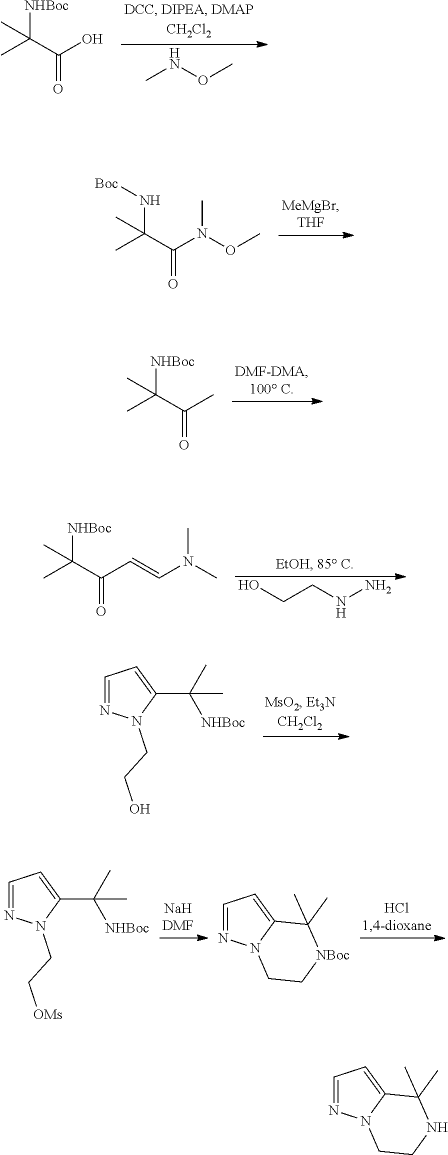

### Step 1: Tert-butyl (1-(methoxy(methyl)amino)-2-methyl-1-oxopropan-2-yl)carbamate

Under Ar, to a solution of 2-((tert-butoxycarbonyl)amino)-2-methylpropanoic acid (10.0 g, 49.2 mmol), N,O-dimethylhydroxylamine hydrochloride (5.76 g, 59.0 mmol), DMAP (7.21 g, 59.0 mmol) and DIPEA (10.3 mL, 59.0 mmol) in CH₂Cl₂ (200 mL) was added DCC (12.2 g, 59.0 mmol). The reaction mixture was stirred at RT for 40 h. The RM was quenched with a sat. aq. NaHCO₃ solution and extracted with EtOAc (×2). The combined organic layers were washed with a sat. aq. NaHCO₃ solution then dried over (Na₂SO₄), filtered, and concentrated in vacuo. The crude residue was purified by normal phase chromatography (eluent: EtOAc in c-hexane 0 to 45%) to give the title compound as a white solid. UPLC-MS-2b: Rt=0.83 min; MS m/z [M+H]⁺ 246.8.

### Step 2: Tert-butyl (2-methyl-3-oxobutan-2-yl)carbamate

Under Ar, to a solution of tert-butyl (1-(methoxy(methyl)amino)-2-methyl-1-oxopropan-2-yl)carbamate (Step 1, 6.23 g, 25.3 mmol) in THF (100 mL) was added a solution of MeMgBr (3M in Et₂O, 25.3 mL, 76.0 mmol) at 0° C. The reaction mixture was stirred at RT for 3 h. The RM was quenched with a sat. aq. NH₄Cl solution and extracted with EtOAc (×2). The combined organic layers were washed with a sat. aq. NH₄Cl solution then dried (Na₂SO₄), filtered, and concentrated in vacuo. The crude residue was purified by normal phase chromatography (eluent: EtOAc in c-hexane 0 to 59%) to give the title compound as a white solid. UPLC-MS-2b: Rt=0.87 min; MS m/z [M+H]⁺ 202.1.

### Step 3: Tert-butyl (E)-(5-(dimethylamino)-2-methyl-3-oxopent-4-en-2-yl)carbamate

A mixture of tert-butyl (2-methyl-3-oxobutan-2-yl)carbamate (Step 2, 3.34 g, 16.6 mmol) in DMF-DMA (1-1, 44.4 mL, 332 mmol) was stirred at 100° C. for 16 h. The reaction mixture was cooled to RT then concentrated in vacuo. And the crude residue was purified by normal phase chromatography (eluent: EtOAc in c-hexane 0 to 100%) to give the title compound as a brown oil. UPLC-MS-2b: Rt=0.86 min; MS m/z [M+H]⁺ 257.2.

### Step 4: Tert-butyl (2-(1-(2-hydroxyethyl)-1H-pyrazol-5-yl)propan-2-yl)carbamate

To a solution of tert-butyl (E)-(5-(dimethylamino)-2-methyl-3-oxopent-4-en-2-yl)carbamate (Step 3, 1.30 g, 5.07 mmol) in EtOH (10 mL) was added 2-hydrazinoethan-1-ol (1.03 mL, 15.2 mmol). The reaction mixture was stirred at 80° C. for 20 h. The RM was cooled to RT, concentrated in vacuo and the crude residue was purified by preparative HPLC (RP-HPLC-3: mobile phase: A: water+7.3 mM NH₄OH, B: acetonitrile; gradient: 5 to 100% B in 20 min) to give the title compound as a regioisomeric mixture as a yellow oil. UPLC-MS-2a: Rt=0.86 min; MS m/z [M+H]⁺ 270.2.

### Step 5: 2-(5-(2-((Tert-butoxycarbonyl)amino)propan-2-yl)-1H-pyrazol-1-yl)ethyl methanesulfonate

Under Ar, to a solution of tert-butyl (2-(1-(2-hydroxyethyl)-1H-pyrazol-5-yl)propan-2-yl)carbamate (Step 4, 885 mg, 3.29 mmol) in CH₂Cl₂ (20 mL) were added methanesulfonic anhydride (1.15 g, 6.57 mmol) and Et₃N (2.29 mL, 16.4 mmol) at 0° C. The reaction mixture was stirred at RT for 1 h before concentrating in vacuo. The crude residue was purified by normal phase chromatography (eluent: MeOH in CH₂Cl₂ 0 to 7.7%) to give the title compound as a regioisomeric mixture as a yellow oil. UPLC-MS-2a: Rt=0.87/0.88 min; MS m/z [M+H]⁺ 348.1.

### Step 6: Tert-butyl 4,4-dimethyl-6,7-dihydropyrazolo[1,5-a]pyrazine-5(4H)-carboxylate

Under Ar, to a solution of 2-(5-(2-((tert-butoxycarbonyl)amino)propan-2-yl)-1H-pyrazol-1-yl)ethyl methanesulfonate (Step 5, 1.11 g, 3.19 mmol) in DMF (20 mL) was added NaH (50% in mineral oil, 166 mg, 4.15 mmol) and the reaction mixture was stirred at RT for 2 h. The reaction was quenched with a sat. aq. NaHCO₃ solution and extracted with EtOAc (×2). The combined organic layers were washed with a sat. aq. NaHCO₃ solution, dried (Na₂SO₄), filtered and concentrated in vacuo. The crude residue was purified by preparative HPLC (RP-HPLC-3: mobile phase: A: water+7.3 mM NH₄OH, B: acetonitrile; gradient: 5 to 100% B in 20 min) to give the title compound as a yellow oil. UPLC-MS-2a: Rt=1.06 min; MS m/z [M+H]⁺ 252.1.

### Step 7: 4,4-Dimethyl-4,5,6,7-tetrahydropyrazolo[1,5-a]pyrazine dihydrochloride

A mixture of tert-butyl 4,4-dimethyl-6,7-dihydropyrazolo[1,5-a]pyrazine-5(4H)-carboxylate (Step 6, 250 mg, 1.00 mmol) and HCl (4M in 1,4-dioxane, 2.49 mL, 9.95 mmol) was stirred at RT for 16 h. The reaction mixture was concentrated in vacuo to give the title compound as a white solid. ¹H NMR (400 MHz, DMSO-d₆) δ 10.38 (br s, 2H), 8.65 (br s, 1H), 7.48 (d, 1H), 6.33 (d, 1H), 4.33 (t, 2H), 3.75-3.65 (m, 2H), 1.69 (s, 6H).

### Intermediate A64: Benzyl 8,8-dimethyl-1-oxa-4,9-diazaspiro[5.5]undecane-4-carboxylate

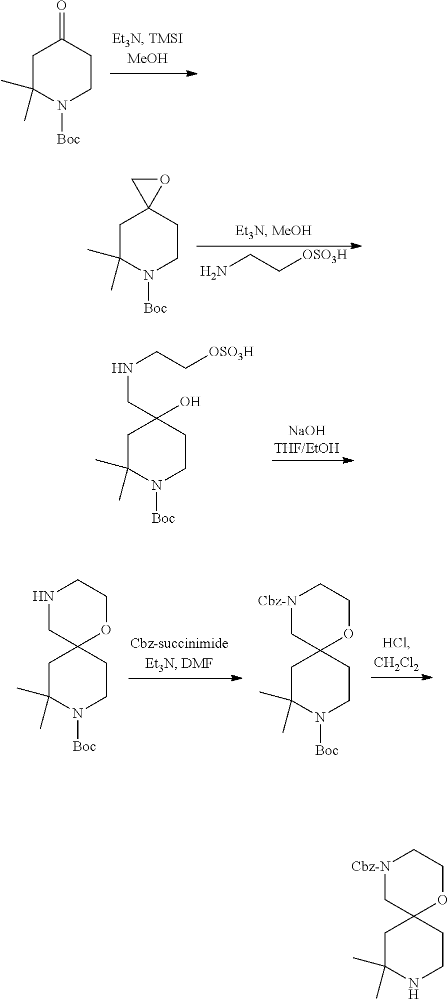

### Step 1: Tert-butyl 5,5-dimethyl-1-oxa-6-azaspiro[2.5]octane-6-carboxylate

To a suspension of sodium hydride (1.32 g, 32.9 mmol) in DMF (30 mL) at 0° C. under N₂ atmosphere was added trimethylsulfoxonium iodide (7.26 g, 32.9 mmol) and the mixture was stirred at RT for 45 min. The reaction mixture was cooled to 0° C. and a solution of 1-boc-2,2-dimethylpiperidin-4-one (5.00 g, 21.9 mmol) in DMF (20 mL) was added dropwise and the RM was stirred at RT for 1 h. After completion of the reaction, the reaction mixture was quenched with cold water and extracted with EtOAc. The combined organic extracts were washed with water, brine, dried (Na₂SO₄), filtered and concentrated under vacuum to afford the title compound as orange oil which was directly used in the next step without further purification. UPLC-MS-5: Rt=1.67 min; MS m/z [M+H]⁺ 242.4.

### Step 2: Tert-butyl 4-hydroxy-2,2-dimethyl-4-(((2-(sulfooxy)ethyl)amino)methyl)piperidine-1-carboxylate

To a solution of tert-butyl 5,5-dimethyl-1-oxa-6-azaspiro[2.5]octane-6-carboxylate (step 1, 5.00 g, 20.7 mmol) in MeOH:Water (5:1) (60 mL) at RT was added 2-aminoethylhydrogensulfate (5.84 g, 41.4 mmol) followed by Et₃N (5.76 g, 41.4 mmol) and the reaction mixture was stirred at 50° C. for 16 h. After completion of reaction, the precipitated white solid was filtered and the filtrate was concentrated under reduced pressure to afford a crude residue which was triturated in 10% MeOH in CH₂Cl₂ and filtered through a pad of celite. The filtrate was then concentrated under reduced pressure to give the title compound as colourless oil which was directly used in the next step without further purification. UPLC-MS-5: Rt=1.31 min; MS m/z [M−H]⁻ 381.5.

### Step 3: Tert-butyl 8,8-dimethyl-1-oxa-4,9-diazaspiro[5.5]undecane-9-carboxylate

To a solution of tert-butyl 4-hydroxy-2,2-dimethyl-4-(((2-(sulfooxy)ethyl)amino)methyl)piperidine-1-carboxylate (step 2, 7.90 g, 16.8 mmol) in THF (70 mL) and EtOH (2.5 mL) was added NaOH (2.47 g, 61.9 mmol) and the reaction mixture was stirred under reflux for 15 h. After completion of the reaction, the mixture was cooled to RT, filtered through a pad of celite and the filtrate was concentrated under vacuum. The residue was diluted with EtOAc and washed with water, brine dried (Na₂SO₄), filtered and concentrated under reduced pressure. The crude residue was purified by normal phase chromatography on neutral alumina (eluent: MeOH in CH₂Cl₂ 2 to 3%) to give the title compound as orange gum. UPLC-MS-5: Rt=1.44 min; MS m/z [M+H]⁺ 285.4.

### Step 4: 4-Benzyl 9-(tert-butyl) 8,8-dimethyl-1-oxa-4,9-diazaspiro[5.5]undecane-4,9-dicarboxylate

To a solution of tert-butyl 8,8-dimethyl-1-oxa-4,9-diazaspiro[5.5]undecane-9-carboxylate (step 3, 5.80 g, 22.8 mmol) and Et₃N (10.5 mL, 75.2 mmol) in dry DMF (70 mL) was added at 0° C. under nitrogen atmosphere N-(benzyloxycarbonyloxy)succinimide (6.20 g, 25.1 mmol) and the reaction mixture was stirred at RT for 15 h. The reaction mixture was poured into ice-cold water and extracted with EtOAc. The combined organic extracts were washed with cold water, brine, dried (Na₂SO₄), filtered and concentrated. The crude residue was purified by normal phase chromatography (eluent: EtOAc in hexane 20 to 25%) to give the title compound. UPLC-MS-5: Rt=2.16 min; MS m/z [M+H]⁺ 419.5.

### Step 5: Benzyl 8,8-dimethyl-1-oxa-4,9-diazaspiro[5.5]undecane-4-carboxylate

To a solution od 4-benzyl 9-(tert-butyl) 8,8-dimethyl-1-oxa-4,9-diazaspiro[5.5]undecane-4,9-dicarboxylate (step 4, 4.50 g, 10.7 mmol) in CH₂Cl₂ (50 mL) at 0° C. under nitrogen atmosphere was added HCl (4M in dioxane, 20 mL) and the reaction mixture was stirred at RT for 3 h. The reaction mixture was concentrated under reduced pressure and co-distilled with CH₂Cl₂ to obtain a crude residue which was purified by reverse phase chromatography (eluent: CH₃CN in H₂O containing 0.1% NH₃ 40 to 45%) to give the title compound. UPLC-MS-5: Rt=1.34 min; MS m/z [M+H]⁺ 319.3.

### Intermediate A65: 1-(1-Oxa-4,9-diazaspiro[5.5]undecan-4-yl)ethan-1-one

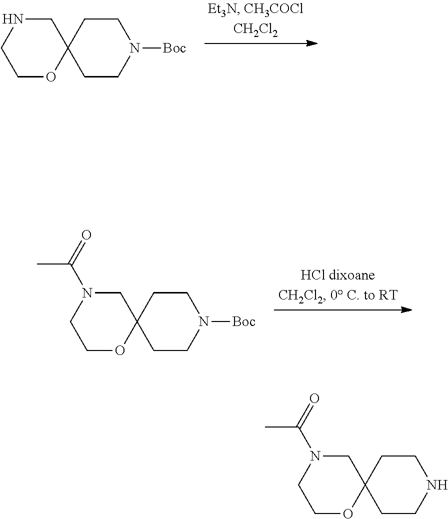 [SMILES: CC(C)(C)OC(=O)N1CCC2(CC1)CNCCO2 ; CC(=O)N1CCOC2(CCN(C(=O)OC(C)(C)C)CC2)C1 ; CC(=O)N1CCOC2(CCNCC2)C1]

### Step 1: Tert-butyl 4-acetyl-1-oxa-4,9-diazaspiro[5.5]undecane-9-carboxylate

To a solution of tert-butyl 1-oxa-4,9-diazaspiro[5.5]undecane-9-carboxylate (CAS [930785-40-3], 1.50 g, 5.80 mmol) in CH₂Cl₂ (15 mL) at 0° C. under nitrogen atmosphere were added Et₃N (0.97 mL, 7.00 mmol) followed by acetyl chloride (0.50 mL, 7.02 mmol) and the reaction mixture was stirred at RT for 5 h. After completion of the reaction, the reaction mixture was quenched with water, extracted with EtOAc. The combined organic layer was dried (Na₂SO₄), filtered and concentrated under vacuum. The crude residue was purified by normal phase chromatography (eluent: MeOH in CH₂Cl₂ 0 to 5%) to give the title compound. UPLC-MS-5: Rt=1.47 min, MS m/z [M+H]⁺ 299.4.

### Step 2: 1-(1-Oxa-4,9-diazaspiro[5.5]undecan-4-yl)ethan-1-one

To a solution of tert-butyl 4-acetyl-1-oxa-4,9-diazaspiro[5.5]undecane-9-carboxylate (Step 1, 1.50 g, 5.36 mmol) in CH₂Cl₂ (10 mL) at 0° C. was added HCl (4M in dioxane, 5 mL) and the reaction mixture was stirred at RT for 2 h. The RM was then concentrated under vacuum to afford the title product as a HCl salt. The salt was dissolved in MeOH (10 mL), tetraalkyl ammonium carbonate resin (Sigma Aldrich cat. 540293, 1.00 g) was added and the mixture was stirred at RT for 2 h. The mixture was then filtered through Millipore and the filtrate was concentrated under reduced pressure to give the title compound. 1H NMR (400 MHz, CDCl₃) δ 3.73-3.66 (m, 4H), 3.55 (m, 2H), 3.36 (m, 4H), 3.23 (m, 2H), 2.09 (s, 3H), 1.98 (m, 2H).

### Intermediate A66: 3,7,7-Trimethyl-1-oxa-3,8-diazaspiro[4.5]decan-2-one

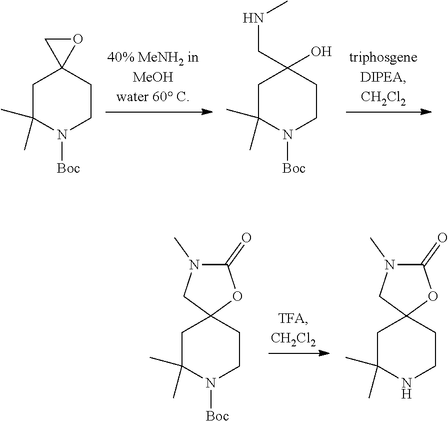 [SMILES: CC(C)(C)OC(=O)N1CCC2(CO2)CC1(C)C ; CNCC1(O)CCN(C(=O)OC(C)(C)C)C(C)(C)C1 ; CN1CC2(CCN(C(=O)OC(C)(C)C)C(C)(C)C2)OC1=O ; CN1CC2(CCNC(C)(C)C2)OC1=O]

### Step 1: Tert-butyl 4-hydroxy-2,2-dimethyl-4-((methylamino)methyl)piperidine-1-carboxylate

Tert-butyl 5,5-dimethyl-1-oxa-6-azaspiro[2.5]octane-6-carboxylate (Step 1 in the synthesis of Intermediate A64, 5.10 g, 21.1 mmol) was dissolved in water (50 mL) and methyl amine (40% in MeOH, 50 mL) was added at 0° C. The reaction mixture was stirred at 60° C. for 4 h in a sealed tube. Water was added and the mixture was extracted with EtOAc. The combined organic extracts were washed with brine, dried (Na₂SO₄), filtered and concentrated under vacuum to afford the title compound which was directly used in the next step without further purification. UPLC-MS-5: Rt=1.27 min; MS m/z [M+H]⁺ 273.

### Step 2: Tert-butyl 3,7,7-trimethyl-2-oxo-1-oxa-3,8-diazaspiro[4.5]decane-8-carboxylate

To a solution of tert-butyl 4-hydroxy-2,2-dimethyl-4-((methylamino)methyl)piperidine-1-carboxylate (Step 1, 4.80 g, 17.6 mmol) CH₂Cl₂ (50 mL) at 0° C. under nitrogen atmosphere was added DIPEA (6.82 g, 52.9 mmol) and triphosgene (2.61 g, 8.81 mmol) and the reaction mixture was stirred at RT for 3 h. Water was added and the mixture was extracted with CH₂Cl₂. The combined organic extracts were washed with brine, dried (Na₂SO₄), filtered and concentrated under vacuum. The crude residue was purified by normal phase chromatography (eluent: 50 to 60% EtOAc in n-Hexane) to give the title compound as colourless gum. UPLC-MS-5: Rt=1.56 min; MS m/z [M+H]⁺ 299.2.

### Step 3: 3,7,7-Trimethyl-1-oxa-3,8-diazaspiro[4.5]decan-2-one

To a solution of tert-butyl 3,7,7-trimethyl-2-oxo-1-oxa-3,8-diazaspiro[4.5]decane-8-carboxylate (Step 2, 3.20 g, 10.7 mmol) in CH₂Cl₂ (30 mL) cooled at 0° C. was added HCl (4M in dioxane, 32 mL) and the reaction mixture was stirred at RT for 2 h. After completion of the reaction, the RM was concentrated under vacuum and co-distilled with CH₂Cl₂. The crude residue was dissolved in MeOH (10 mL), tetralkyl ammonium carbonate (MP-carbonate, 1.50 g) was added and the mixture was swirled at 30° C. for 15 min. The mixture was filtered through Millipore, washed with MeOH and the filtrate was concentrated under reduced pressure to give the title product as a white solid which was directly used in the next step without further purification. UPLC-MS-5: Rt=1.24 min; MS m/z [M+H]⁺ 199.1.

### Intermediate A67: 6,6-Dimethyl-2-(oxetan-3-yl)-2,7-diazaspiro[3.5]nonane

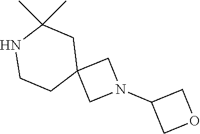

### Step 1: Tert-butyl 4-cyano-2,2-dimethylpiperidine-1-carboxylate

To a solution of tert-butyl 2,2-dimethyl-4-oxopiperidine-1-carboxylate (500 mg, 2.20 mmol) in DME (15.5 mL) under inert atmosphere at RT were added (p-tolylsulfonyl)methyl isocyanide (537 mg, 2.75 mmol) and EtOH (0.22 mL, 3.74 mmol). To the reaction mixture cooled at 0° C. was added dropwise tert-BuOK (1M in THF, 5.65 mL, 5.65 mmol) over a period of 5 min. Then the reaction mixture was stirred for 10 min at 0° C., warmed to RT and stirred at 50° C. overnight. After completion, the reaction was cooled to RT, water was added and the aqueous layer was extracted twice with Et₂O and twice with EtOAc. The combined organic layers were dried (Na₂SO₄), filtered and concentrated under reduced pressure. The crude residue was purified by normal phase chromatography (eluent: 0 to 25% EtOAc in c-hexane) to give the title compound as a light yellow gum. UPLC-MS-2a: Rt=1.01 min; MS m/z [M-Boc+H]⁺139.2.

### Step 2: 1-(Tert-butyl) 4-ethyl 4-cyano-2,2-dimethylpiperidine-1,4-dicarboxylate

To tert-butyl 4-cyano-2,2-dimethylpiperidine-1-carboxylate (Step 1, 2.00 g, 8.39 mmol) in THF (25.2 mL) at −78° C. under inert atmosphere was added dropwise LiHMDS (1M in THF, 16.8 mL, 16.8 mmol) followed after 1 h at −78° C. by a dropwise addition of a solution of ethyl chloroformate (1.61 mL, 16.8 mmol) in THF (1 mL). The reaction mixture was stirred for 1 h at −78° C. After complete conversion, the reaction mixture was poured into a sat. aq. NaHCO₃ solution and extracted twice with EtOAc. The combined organic layers were washed with brine, dried (Na₂SO₄), filtered and concentrated under reduced pressure. The crude residue was purified by normal phase chromatography (eluent: 0 to 25% EtOAc in c-hexane) to give the title compound as a light yellow oil. UPLC-MS-2a: Rt=1.11 min; MS m/z [M-Boc+H]⁺211.3.

### Step 3: Tert-butyl 4-cyano-4-(hydroxymethyl)-2,2-dimethylpiperidine-1-carboxylate

To an ice-cooled solution of 1-(tert-butyl) 4-ethyl 4-cyano-2,2-dimethylpiperidine-1,4-dicarboxylate (Step 2, 1.99 g, 6.40 mmol) in MeOH (10 mL) under inert atmosphere was added portionwise NaBH₄ (0.29 g, 7.68 mmol). The reaction mixture was stirred at 0° C. for 30 min and at RT for 1 h. After complete conversion, the reaction mixture was cooled at 0° C. then diluted with TBME and water followed by addition of 4N HCl to adjust the pH around 2. The aqueous layer was extracted with TBME. The combined organic layers were washed with a sat. aq. solution of NaHCO₃ then brine, dried (Na₂SO₄), filtered and concentrated under reduced pressure. The crude residue was purified by normal phase chromatography (eluent: 0 to 25% EtOAc in c-hexane) to give the title compound as a colorless oil. UPLC-MS-2a: Rt=0.92 min; MS m/z [M-Boc+H]⁺169.2.

### Step 4: Tert-butyl 4-cyano-2,2-dimethyl-4-((tosyloxy)methyl)piperidine-1-carboxylate

To a solution of tert-butyl 4-cyano-4-(hydroxymethyl)-2,2-dimethylpiperidine-1-carboxylate (Step 3, 5.00 g, 18.6 mmol) in CH₂Cl₂ (30.2 mL) at 0° C. under inert atmosphere were added Et₃N (3.10 mL, 22.4 mmol) and DMAP (0.11 g, 0.93 mmol), followed by a solution of p-toluenesulfonyl chloride (4.26 g, 22.4 mmol) in CH₂Cl₂ (20 mL). The reaction mixture was stirred at 0° C. for 1 h and at RT for 16 h then diluted with water and extracted with CH₂Cl₂. The organic layer was dried (Na₂SO₄), filtered and concentrated under reduced pressure. The crude residue was purified by normal phase chromatography (eluent: 0 to 40% EtOAc in c-hexane) to give the title compound as a colorless oil. UPLC-MS-4: Rt=1.22 min; MS m/z [M-Boc+H]⁺323.3.

### Step 5: Tert-butyl 6,6-dimethyl-2,7-diazaspiro[3.5]nonane-7-carboxylate

To a solution of lithium aluminium hydride (1M in THF, 26.2 mL, 26.2 mmol) in THF (25.7 mL) at 0° C. under inert atmosphere was added dropwise a solution of tert-butyl 4-cyano-2,2-dimethyl-4-((tosyloxy)methyl)piperidine-1-carboxylate (Step 4, 7.39 g, 17.5 mmol) in THF (18.4 mL) and the reaction mixture was stirred at RT for 16 h. The reaction mixture was cooled at 0° C. and quenched carefully with water (1.80 mL), NaOH (15%, 1.80 mL), water (5.0 mL) and THF (30 mL). The mixture was stirred at 0° C. for 30 min, filtered, the filtered cake was washed with CH₂Cl₂. Then the filtrate was concentrated under reduced pressure. The residue was dissolved with CH₂Cl₂, dried (Na₂SO₄), filtered and concentrated under reduced pressure to give the title compound which was used without purification in the next step. UPLC-MS-4: Rt=0.43 min; MS m/z [M-Boc+H]⁺155.3.

### Step 6: Tert-butyl 6,6-dimethyl-2-(oxetan-3-yl)-2,7-diazaspiro[3.5]nonane-7-carboxylate

To a solution of tert-butyl 6,6-dimethyl-2,7-diazaspiro[3.5]nonane-7-carboxylate (Step 5, 4.37 g, 17.0 mmol) in DCE (68.8 mL) was added oxetan-3-one (1.68 mL, 26.2 mmol) and the reaction mixture was stirred for 3 h. Then sodium triacetoxyborohydride (5.93 g, 28.0 mmol) was added and the reaction mixture was stirred at RT for 16 h. The RM was quenched with a sat. aq. NaHCO₃ solution and extracted with CH₂Cl₂. The organic layer was dried (Na₂SO₄), filtered and concentrated under reduced pressure. The crude residue was purified by normal phase chromatography (eluent: 0 to 10% CH₃OH in CH₂Cl₂) to give the title compound as a white solid. UPLC-MS-4: Rt=0.44 min; MS m/z [M-Boc+H]⁺211.3.

### Step 7: 6,6-Dimethyl-2-(oxetan-3-yl)-2,7-diazaspiro[3.5]nonane

To a solution of tert-butyl 6,6-dimethyl-2-(oxetan-3-yl)-2,7-diazaspiro[3.5]nonane-7-carboxylate (Step 6, 3.65 g, 11.8 mmol) in CH₂Cl₂ (42.8 mL) was added TFA (13.6 mL, 176 mmol) and the reaction mixture was stirred at RT for 2 h. The RM was concentrated under reduced pressure, the residue was dissolved in dioxane, frozen and lyophilized to give the title compound as a TFA salt. The material was dissolved in MeOH (500 mL) and MP-carbonate (235 mmol, 80 g) was added. The mixture was swirled at RT for 1 h then was filtered, washed with MeOH and the filtrate was concentrated under reduced pressure to give the title compound as a colorless oil. UPLC-MS-4: Rt=0.13 min; MS m/z [M+H]⁺ 211.3.

### Intermediate A68: 2-(Oxetan-3-yl)-2,10-diazadispiro[3.1.3⁶.3⁴]dodecane

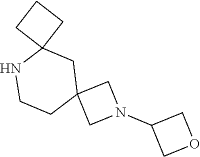 [SMILES: C1CC2(C1)CC1(CCN2)CN(C2COC2)C1]

The title compound was prepared in 7 steps by a method similar to that of 6,6-dimethyl-2-(oxetan-3-yl)-2,7-diazaspiro[3.5]nonane (Intermediate A67) using tert-butyl 8-oxo-5-azaspiro[3.5]nonane-5-carboxylate (Step 1) instead of tert-butyl 2,2-dimethyl-4-oxopiperidine-1-carboxylate. UPLC-MS-4: Rt=0.13 min; MS m/z [M+H]⁺ 223.3.

### Intermediate A69: 4-(((Tert-butyldiphenylsilyl)oxy)methyl)-2,2-dimethylpiperidine

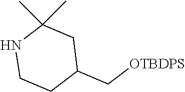

### Step 1: Tert-butyl 4-(hydroxymethyl)-2,2-dimethylpiperidine-1-carboxylate

To a solution of 1-(tert-butoxycarbonyl)-2,2-dimethylpiperidine-4-carboxylic acid (20.0 g, 78.0 mmol) in THF (200 mL) was added CDI (15.1 g, 93.0 mmol) and the mixture was stirred at RT for 1 h under nitrogen atmosphere. The mixture was then cooled to 0° C., a solution of NaBH₄ (5.29 g, 140 mmol) in Water (100 mL) was added and the reaction mixture was stirred at RT for 2 h. The reaction mixture was cooled to 0° C., AcOEt was added and a solution of aqueous HCl (0.5N) was added. The layers were separated and the organic layer was neutralized with a solution of aqueous NaOH (1N), extracted with EtOAc and the combined organic extracts were dried (phase separator) and concentrated under vacuum to give the title compound which was used without further purification in the next step. UPLC-MS-4: Rt=1.55 min; MS m/z [M−100]⁺ 144.1.

### Step 2: Tert-butyl 4-(((tert-butyldiphenylsilyl)oxy)methyl)-2,2-dimethylpiperidine-1-carboxylate

To a solution of tert-butyl 4-(hydroxymethyl)-2,2-dimethylpiperidine-1-carboxylate (Step 1, 1.20 g, 10.3 mmol) in CH₂Cl₂ (12 mL) at 0° C. under nitrogen atmosphere were added imidazole (0.67 g, 20.6 mmol) and tert-butyldiphenylsilyl chloride (1.50 mL, 12.3 mmol) and the reaction mixture was allowed to reach RT and stirred for 1.5 h. The reaction mixture was poured in water and extracted with EtOAc. The combined organic extracts were washed with water, brine, dried (Na₂SO₄), filtered and concentrated under vacuum to give the title compound which was used in the next step without further purification. ¹H NMR (400 MHz, Chloroform-d) δ 7.67 (d, 4H), 7.51-7.32 (m, 6H), 3.66 (m, 1H), 3.51 (d, 2H), 3.26 (m, 1H), 1.98-1.80 (m, 2H), 1.70-1.59 (m, 2H), 1.48 (s, 9H), 1.4 (m 1H), 1.22 (s, 3H), 1.09 (s, 12H).

### Step 3: 4-(((Tert-butyldiphenylsilyl)oxy)methyl)-2,2-dimethylpiperidine

To a solution of tert-butyl 4-(((tert-butyldiphenylsilyl)oxy)methyl)-2,2-dimethylpiperidine-1-carboxylate (Step 2, 2.70 g, 5.61 mmol) in CH₂Cl₂ (27 mL) at 0° C. and under nitrogen atmosphere was added TFA (20 mL) and the reaction mixture was stirred at RT for 2 h. The RM was concentrated under vacuum, quenched with a sat. aq. solution of NaHCO₃ and extracted with EtOAc. The combined organic extracts were washed with water, brine, dried (Na₂SO₄), filtered and concentrated under vacuum. The crude residue was purified by reverse phase chromatography (eluent: 55 to 65% CH₃CN in H₂O containing 0.025% NH₃) to give the title product. UPLC-MS-5: Rt=1.89 min; MS m/z [M+H]⁺ 382.4.

### Method-A70 for the preparation of A70: (R)-4-((2,2-Dimethylpiperidin-4-yl)methyl)morpholine

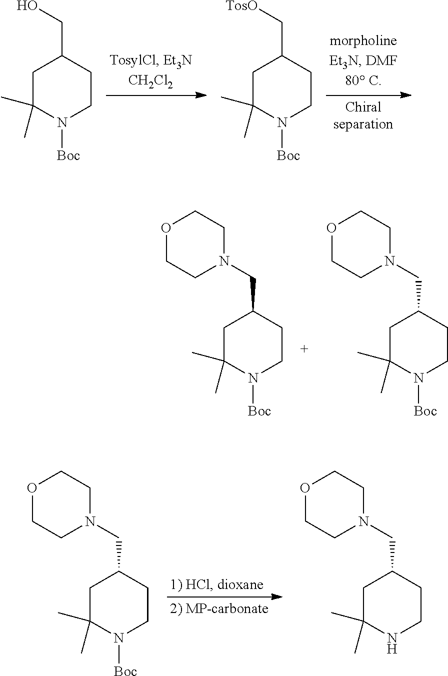

### Step 1: Tert-butyl 2,2-dimethyl-4-((tosyloxy)methyl)piperidine-1-carboxylate

To a solution of tert-butyl 4-(hydroxymethyl)-2,2-dimethylpiperidine-1-carboxylate (Step 1 in the synthesis of Intermediate A69, 17.2 g, 70.5 mmol) in CH₂Cl₂ (380 mL) under argon atmosphere was added at 0° C. Et₃N (19.5 mL, 141 mmol) followed by Tosyl-Cl (14.1 g, 74.0 mmol). The reaction mixture was stirred at RT for 16 h. The reaction was poured in a sat. aq. NaHCO₃ solution and extracted with CH₂Cl₂ (×2). The combined organic extracts were washed with brine, dried (phase separator) and concentrated. The crude residue was purified by normal phase chromatography (eluent: EtOAc in n-heptane 0 to 30%) to give the title compound. UPLC-MS-4: Rt=1.32 min; MS m/z [M−100+H]⁺298.2.

### Step 2: Tert-butyl (S)-2,2-dimethyl-4-(morpholinomethyl)piperidine-1-carboxylate and Tert-butyl (R)-2,2-dimethyl-4-(morpholinomethyl)piperidine-1-carboxylate

To morpholine (7.28 g, 84 mmol) were added a solution of tert-butyl 2,2-dimethyl-4-((tosyloxy)methyl)piperidine-1-carboxylate (Step 1, 23.7 g, 59.7 mmol) in DMF (250 mL) and Et₃N (24.8 mL, 179 mmol) and the reaction mixture was stirred at 80° C. for 16 h. Water and EtOAc were added and the layers were separated. The organic layer was washed with brine and dried (Na₂SO₄), filtered and concentrated. The enantiomers were separated by chiral SFC (C-SFC-21; mobile phase: CO₂/[MeOH+0.025% NH₃]: 85/15) to give tert-butyl (S)-2,2-dimethyl-4-(morpholinomethyl)piperidine-1-carboxylate as the first eluting enantiomer; C-SFC-22 (mobile phase: CO₂/[MeOH+0.05% DEA]95/5) Rt=2.14 min, UPLC-MS-4: Rt=0.46 min; MS m/z [M+H]⁺ 313.4 and tert-butyl (R)-2,2-dimethyl-4-(morpholinomethyl)piperidine-1-carboxylate as the second eluting enantiomer: C-SFC-22 (mobile phase: CO₂/[MeOH+0.05% DEA] 95/5) Rt=2.77 min, UPLC-MS-4: Rt=0.46 min; MS m/z [M+H]⁺ 313.4.

### Step 3: (R)-4-((2,2-dimethylpiperidin-4-yl)methyl)morpholine

To a solution of tert-butyl (R)-2,2-dimethyl-4-(morpholinomethyl)piperidine-1-carboxylate (Step 2 second eluting enantiomer, 6.89 g, 22.0 mmol) in dioxane (75 mL) was added HCl (4N in dioxane, 55 mL, 221 mmol) and the reaction mixture was stirred at RT for 16 h. After completion of the reaction, volatils were evaporated. The crude residue was dissolved in Methanol (150 mL), MP-Carbonate (26.8 g, 28.9 mmol) was added and the mixture was swirled at RT for 1 h. The mixture was filtered and the filtrate was concentrated and dried under high vacuum to give the title compound which was used in the next step without purification. ¹H NMR (400 MHz, DMSO-d₆) δ 3.55 (t, 4H), 2.80-2.74 (m, 2H), 2.33-2.24 (m, 4H), 2.06-2.01 (m, 2H), 1.04 (m, 1H), 1.65 (m, 1H), 1.53 (m, 1H), 1.09 (s, 6H), 0.95-0.83 (m, 2H).

Method-A70a: similar to Method-A70 except that NaI (0.5 eq.) was added in Step 2.

Method-A70b: similar to Method-A70 except that Step 2 was performed in CH₃CN at 100° C. under microwave irradiations as described in the synthesis of Intermediate A78.

Method-A70c: similar to Method-A70 except that Step 3 was performed using TFA (10 eq.) in CH₂Cl₂ as described in the synthesis of Intermediate A78.

Method-A70d: similar to Method-A70 except that Step 2 was performed in CH₃CN.

Method-A70e: similar to Method-A70 except that Step 2 was performed using NaH (60% in mineral oil) instead of Et₃N.

The following examples A71 to A77 were prepared using analogous methods to method-A70 from commercially available precursors (in Step 2).

| | | Method, precursors (Step) | |
|---|---|---|---|
| | | and chiral separation | |
| Intermediate | Structure | conditions | Characterizing data |
| A71 | 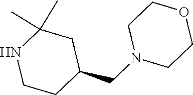 | Using Method-A70 from tert- butyl (S)-2,2-dimethyl-4- (morpholinomethyl)piperidine- 1-carboxylate (first eluting enantiomer in Step 2) (Step 3) | ¹H NMR (400 MHz, DMSO-d₆) δ 3.55 (t, 4H), 2.80-2.74 (m, 2H), 2.33-2.24 (m, 4H), 2.06-2.01 (m, 2H), 1.04 (m, 1H), 1.65 (m, 1H), 1.53 (m, 1H), 1.09 (s, 6H), 0.95-0.83 (m, 2H). |
| | (S)-4-((2,2- | | |
| | dimethylpiperidin-4- | | |
| | yl)methyl)morpholine | | |
| A72 | 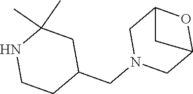 [SMILES: CC1(C)CC(CN2CC3CC(C2)O3)CCN1] | Using Method-A70ac from 6- oxa-3- azabicyclo[3.1.1]heptane (Step 2) without chiral separation in Step 2 | UPLC-MS-4: Rt = 0.12 min; MS m/z [M + H]⁺ 225.3. |
| | 3-((2,2- | | |
| | dimethylpiperidin-4- | | |
| | yl)methyl)-6-oxa-3- | | |
| | azabicyclo[3.1.1]heptane | | |
| A73 | 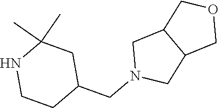 [SMILES: CC1(C)CC(CN2CC3COCC3C2)CCN1] | Using Method-A70b from hexahydro-1H-furo[3,4- c]pyrrole (Step 2) without chiral separation in Step 2 | UPLC-MS-4: Rt = 0.12 min; MS m/z [M + H]⁺ 239.3. |
| | 5-((2,2- | | |
| | dimethylpiperidin-4- | | |
| | yl)methyl)hexahydro-1H- | | |
| | furo[3,4-c]pyrrole | | |
| A74 | 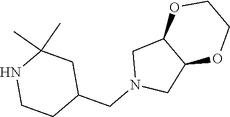 [SMILES: CC1(C)CC(CN2C[C@@H]3OCCO[C@@H]3C2)CCN1] | Using Method-A70c from cis- (4aR,7aS)-hexahydro-2H- [1,4]dioxino[2,3-C]pyrrole hydrochloride (Step 2) without chiral separation in Step 2 | UPLC-MS-4: Rt = 0.12 min; MS m/z [M + H]⁺ 255.0. |
| | (4aR,7aS)-6-((2,2- | | |
| | dimethylpiperidin-4- | | |
| | yl)methyl)hexahydro-5H- | | |
| | [1,4]dioxino[2,3-c]pyrrole | | |
| A75 | 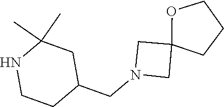 [SMILES: CC1(C)CC(CN2CC3(CCCO3)C2)CCN1] | Using Method-A70acd from 5- oxa-2-azaspiro[3.4]octane (Step 2) without chiral separation in Step 2 | UPLC-MS-4: Rt = 0.13 min; MS m/z [M + H]⁺ 239.3. |
| | 2-((2,2- | | |
| | dimethylpiperidin-4- | | |
| | yl)methyl)-5-oxa-2- | | |
| | azaspiro[3.4]octane | | |
| A76 | 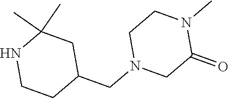 [SMILES: CN1CCN(CC2CCNC(C)(C)C2)CC1=O] | Using Method-A70a from 1- methylpiperazin-2-one hydrochloride (CAS [109384- 27-2]) without chiral separation in Step 2 | UPLC-MS-4: Rt = 0.15 min; MS m/z [M + H]⁺ 240.4. |
| | 4-((2,2- | | |
| | dimethylpiperidin-4- | | |
| | yl)methyl)-1- | | |
| | methylpiperazin-2-one | | |
| A77 | 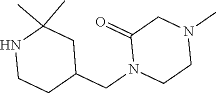 [SMILES: CN1CCN(CC2CCNC(C)(C)C2)C(=O)C1] | Using Method-A70e from 4- methylpiperazin-2-one (CAS [34770-60-0]) without chiral separation in Step 2 | UPLC-MS-4: Rt = 0.14 min; MS m/z [M + H]⁺ 240.3. |
| | 1-((2,2- | | |
| | dimethylpiperidin-4- | | |
| | yl)methyl)-4- | | |
| | methylpiperazin-2-one | | |

### Intermediate A78: (R)-1-((2,2-dimethylpiperidin-4-yl)methyl)-4-(oxetan-3-yl)piperazine

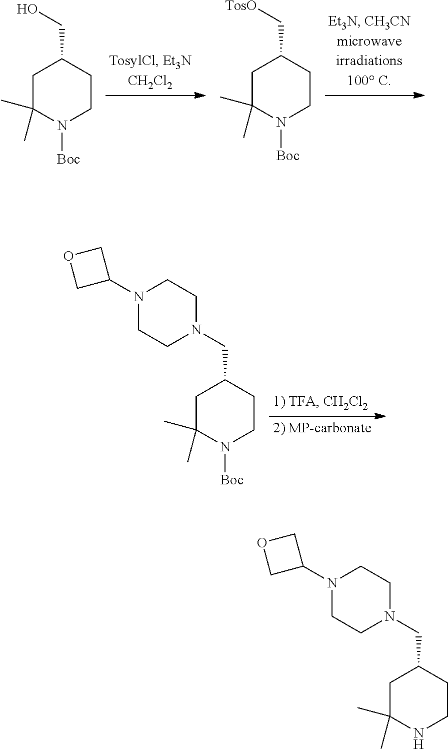

### Step 1: Tert-butyl (R)-2,2-dimethyl-4-((tosyloxy)methyl)piperidine-1-carboxylate

To a solution of tert-butyl (R)-4-(hydroxymethyl)-2,2-dimethylpiperidine-1-carboxylate (Intermediate described In the synthesis of Intermediate A85 (Step 2), 26.8 g, 105 mmol) in CH₂Cl₂ (450 mL) under argon atmosphere was added at 0° C. Et₃N (30.0 mL, 215 mmol) followed by Tosyl-Cl (21.0 g, 110 mmol). The reaction mixture was stirred at RT for 16 h. The reaction was poured in a sat. aq. NaHCO₃ solution and extracted with CH₂Cl₂ (×2). The combined organic extracts were washed with brine, dried (phase separator) and concentrated. The crude residue was purified by normal phase chromatography (eluent: EtOAc in n-heptane 0 to 20%) to give the title compound. C-SFC-19 (mobile phase: CO₂/[IPA+0.025% NH₃] 85/15): Rt=2.56 min (note: Rt: 2.94 min for tert-butyl (S)-2,2-dimethyl-4-((tosyloxy)methyl)piperidine-1-carboxylate); UPLC-MS-4: Rt=1.32 min; MS m/z [M+H]⁺ 298.2.

### Step 2: Tert-butyl (R)-2,2-dimethyl-4-((4-(oxetan-3-yl)piperazin-1-yl)methyl)piperidine-1-carboxylate

To a solution of 1-(oxetan-3-yl)piperazine (0.76 g, 5.32 mmol) in CH₃CN (16 mL) were added tert-butyl (R)-2,2-dimethyl-4-((tosyloxy)methyl)piperidine-1-carboxylate (Step 1, 2.10 g, 5.07 mmol) and Et₃N (2.12 mL, 15.2 mmol). The reaction mixture was stirred at 100° C. under microwave irradiations for 15 h. The RM was poured into a sat. aq. NaHCO₃ solution and extracted with CH₂Cl₂ (×3). The combined organic extracts were dried (phase separator) and concentrated. The crude residue was purified by normal phase chromatography (eluent: MeOH in CH₂Cl₂ from 0 to 15%) to give the title compound. UPLC-MS-4: Rt=0.48 min; MS m/z [M+H]⁺ 368.4.

### Step 3: (R)-1-((2,2-dimethylpiperidin-4-yl)methyl)-4-(oxetan-3-yl)piperazine

To a solution of tert-butyl (R)-2,2-dimethyl-4-((4-(oxetan-3-yl)piperazin-1-yl)methyl)piperidine-1-carboxylate (Step 2, 1.52 g, 4.05 mmol) in CH₂Cl₂ (15 mL) was added TFA (3.12 mL, 40.5 mmol) and the reaction mixture was stirred at RT for 2 h. The RM was concentrated under reduced pressure. The crude residue was dissolved in MeOH (60 mL), MP-Carbonate (21 g, 60.7 mmol) was added and the mixture was swirled at RT for 1 h. The mixture was filtered and the filtrate was concentrated and dried under high vacuum to give the title compound which was used in the next step without purification. UPLC-MS-4: Rt=0.12 min; MS m/z [M+H]⁺ 268.4.

### Intermediate A79: (4-((2,2-dimethylpiperidin-4-yl)methyl)thiomorpholine 1,1-dioxide

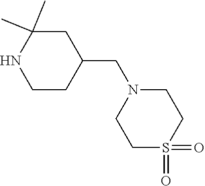 [SMILES: CC1(C)CC(CN2CCS(=O)(=O)CC2)CCN1]

### Step 1: Tert-butyl 4-(aminomethyl)-2,2-dimethylpiperidine-1-carboxylate

In a 200 mL autoclave was introduced tert-butyl 2,2-dimethyl-4-((tosyloxy)methyl)piperidine-1-carboxylate (Step 1 in the synthesis of Intermediate A70, 3.00 g, 7.56 mmol) and NH₃ (7 M in MeOH, 27.0 mL, 189 mmol) and the reaction mixture at 85° C. for 48 h. The RM was concentrated to give the title compound as a white solid. UPLC-MS-4: Rt=0.52 min; MS m/z [M+H]⁺ 243.3.

### Step 2: Tert-butyl 4-((1,1-dioxidothiomorpholino)methyl)-2,2-dimethylpiperidine-1-carboxylate

To a stirred solution of tert-butyl 4-(aminomethyl)-2,2-dimethylpiperidine-1-carboxylate (Step 1, 3.27 g, 13.5 mmol) in EtOH (75 mL) was added under Argon divinyl sulfone (2.70 mL, 27.0 mmol) and NEt₃ (3.76 mL, 27.0 mmol). The reaction mixture was stirred at 80° C. for 16 h. A sat. aq. NaHCO₃ solution was added and the mixture was extracted with EtOAc (×2). The combined organic extracts were washed with a sat. aq. NaHCO₃ solution, dried (Na₂SO₄), filtered and evaporated. The crude residue was purified by normal phase chromatography (eluent: EtOAc in c-hexane from 0 to 100%) to give the title compound as a white solid. UPLC-MS-4: Rt=0.91 min; MS m/z [M+H]⁺ 361.3.

### Step 3: (4-((2,2-Dimethylpiperidin-4-yl)methyl)thiomorpholine 1,1-dioxide

To a stirred solution of tert-butyl 4-((1,1-dioxidothiomorpholino)methyl)-2,2-dimethylpiperidine-1-carboxylate (Step 2, 2.22 g, 6.16 mmol) in dioxane (25 mL) was added HCl (4N in dioxane, 15.4 mL, 61.6 mmol) and the reaction mixture was stirred at RT for 20 h. The RM was concentrated to dryness, dissolved in MeOH (30 mL), MP-carbonate (25.0 g, 18.6 mmol) was added and the mixture was stirred at 40° C. for 1 h. The mixture was filtered over a pad of celite and the filtrate was concentrated. The crude residue was purified by normal phase chromatography on basic Alumina (eluent: MeOH in CH₂Cl₂ from 0 to 10%) to give the title compound as a yellow oil. UPLC-MS-4: Rt=0.12 min; MS m/z [M+H]⁺ 261.3.

### Intermediate A80: 4-((3-Methoxyazetidin-1-yl)methyl)-2,2-dimethylpiperidine

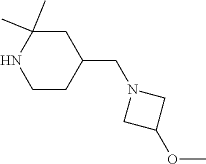

### Step 1: Tert-butyl 4-formyl-2,2-dimethylpiperidine-1-carboxylate

DMSO (620 μL, 8.73 mmol) was added dropwise under an inert atmosphere to a solution of oxalyl chloride (229 μL, 2.62 mmol) in CH₂Cl₂ (3.88 mL) at −78° C. and stirred for 10 mn, then tert-butyl 4-(hydroxymethyl)-2,2-dimethylpiperidine-1-carboxylate (Step 1 in the synthesis of Intermediate A69, 425 mg, 1.75 mmol) in CH₂Cl₂ (1.94 mL) was added and the mixture was stirred at −78° C. for 30 min, then triethylamine (1.46 mL, 10. mmol) was added dropwise and the reaction mixture was stirred at −78° C. for 1 h. The RM was quenched with water and extracted with CH₂Cl₂. The combined organic layers were dried (Na₂SO₄), filtered and concentrated under reduced pressure. The crude residue was purified by normal phase chromatography (eluent: 0 to 50% EtOAc in c-hexane) to give the title compound as a white solid. ¹H NMR (400 MHz, DMSO-d₆) δ 9.60 (d, 1H), 3.50-3.39 (m, 1H), 3.30-3.23 (m, 1H), 2.73-2.59 (m, 1H), 1.81-1.68 (m, 2H), 1.67-1.55 (m, 2H), 1.40 (s, 3H), 1.39 (s, 9H), 1.32 (s, 3H).

### Step 2: Tert-butyl 4-((3-methoxyazetidin-1-yl)methyl)-2,2-dimethylpiperidine-1-carboxylate

To a solution of 3-methoxyazetidine hydrochloride (151 mg, 1.22 mmol) in DCE (3 mL) under inert atmosphere was added triethylamine (0.17 mL, 1.22 mmol). After stirring for 30 min, tert-butyl 4-formyl-2,2-dimethylpiperidine-1-carboxylate (Step 2, 268 mg, 1.11 mmol) in DCE (1 mL) and MgSO₄ (267 mg, 2.22 mmol) were added and the mixture was stirred at RT for 16 h. Sodium triacetoxyborohydride (377 mg, 1.78 mmol) was added and the reaction mixture was stirred at RT for 24 h. The RM was quenched with a sat. aq. NaHCO₃ solution and extracted with CH₂Cl₂. The combined organic layer was dried (Na₂SO₄), filtered and evaporated. The crude residue was purified by normal phase chromatography (eluent: 0 to 15% CH₃OH in CH₂Cl₂) to give the title compound as a colorless oil. UPLC-MS-4: Rt=0.43 min; MS m/z [M+H]⁺ 313.4.

### Step 3: 4-((3-Methoxyazetidin-1-yl)methyl)-2,2-dimethylpiperidine

To a solution of tert-butyl 4-((3-methoxyazetidin-1-yl)methyl)-2,2-dimethylpiperidine-1-carboxylate (Step 3, 312 mg, 1.00 mmol) in CH₂Cl₂ (3.70 mL) was added TFA (1.15 mL, 15.0 mmol) and the reaction mixture was stirred at RT for 5 h. The mixture was concentrated, the residue was dissolved in dioxane, frozen and lyophilized to give the title compound as a TFA salt. The material was dissolved in MeOH (40 mL), MP-carbonate (8 mmol, 2.8 g) was added and the mixture was swirled at RT for 1 h then was filtered, washed with MeOH and the filtrate was concentrated under reduced pressure to give the title compound as a colorless oil. UPLC-MS-4: Rt=0.22 min; MS m/z [M+H]⁺ 213.4.

### Intermediate A81: (1 S,4S)-5-((2,2-Dimethylpiperidin-4-yl)methyl)-2-oxa-5-azabicyclo[2.2.1]heptane

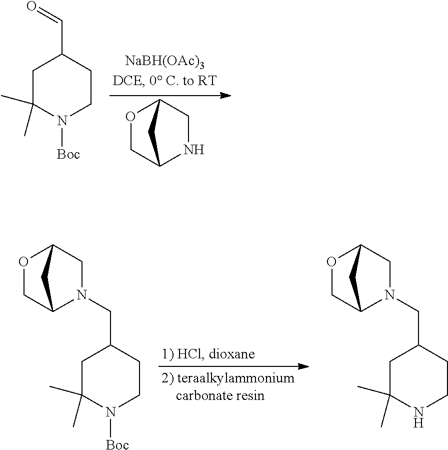

### Step 1: Tert-butyl 4-(((1S,4S)-2-oxa-5-azabicyclo[2.2.1]heptan-5-yl)methyl)-2,2-dimethylpiperidine-1-carboxylate

To a solution of tert-butyl 4-formyl-2,2-dimethylpiperidine-1-carboxylate (described in the synthesis of Intermediate A80 (Step 1), 2.18 g, 8.70 mmol) in dichloroethane (21 mL) was added (1S,4S)-2-oxa-5-azabicyclo[2.2.1]heptane hydrochloride (1.53 g, 11.3 mmol) and the mixture was stirred at RT for 2 h. The mixture was then cooled to 0° C., NaBH(OAc)₃ (2.77 g, 13.05 mmol) was added and RM was allowed to reach RT and stirred for 16 h. The RM was quenched with water, solid NaHCO₃ was added and the mixture was extracted with EtOAc. The combined organic extracts were washed with brine, dried (Na₂SO₄), filtered and concentrated under vacuum to give the title product which was used in the next step without purification. UPLC-MS-4: Rt=0.40 min; MS m/z [M+H]⁺ 325.4.

### Step 2: (1 S,4S)-5-((2,2-Dimethylpiperidin-4-yl)methyl)-2-oxa-5-azabicyclo[2.2.1]heptane

To a solution of tert-butyl 4-(((1S,4S)-2-oxa-5-azabicyclo[2.2.1]heptan-5-yl)methyl)-2,2-dimethylpiperidine-1-carboxylate (Step 1, 2.30 g, 7.08 mmol) in CH₂Cl₂ (21 mL) at 0° C. was added HCl (4M in dioxane, 25 mL) and the reaction mixture was allowed to stir at RT for 3 h. Then the RM was concentrated under reduced pressure and the residue was dissolved in MeOH, tetraalkylammonium carbonate, polymer-bound (2 g) was added at 0° C. and the mixture was swirled at RT for 30 min, the filtered through Millipore and the filtrate was concentrated under vacuum. The crude residue was by normal phase chromatography on basic Alumina (eluent: 0 to 5% MeOH in CH₂Cl₂) to give the title product as yellow oil. UPLC-MS-4: Rt=0.21 min; MS m/z [M+H]⁺ 225.4.

### Intermediate A82: (2,2-Dimethylpiperidin-4-yl)(3-methoxyazetidin-1-yl)methanone

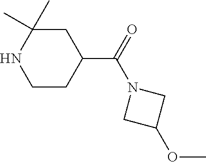

### Step 1: Tert-butyl 4-(3-methoxyazetidine-1-carbonyl)-2,2-dimethylpiperidine-1-carboxylate

To an ice-cooled solution of 1-(tert-butoxycarbonyl)-2,2-dimethylpiperidine-4-carboxylic acid (10.0 g, 38.9 mmol) in CH₂Cl₂ (194 mL) was added under inert atmosphere propylphosphonic anhydride (50% in EtOAc, 22.9 mL, 38.9 mmol) and DIPEA (26.6 mL, 155 mmol). After stirring at for 30 min, the mixture was added to a solution of 3-methoxyazetidine hydrochloride (5.76 g, 46.6 mmol) in CH₂Cl₂ (194 mL) at 0° C. The reaction mixture was stirred and warmed-up to RT over 20 h. The RM was quenched by addition of 1N HCl, then the organic phase was washed with 1N NaOH, dried (Na₂SO₄), filtered and evaporated under reduced pressure. The crude residue was purified by normal phase chromatography (eluent: 0 to 10% CH₃OH in CH₂Cl₂) to give the title compound as a colorless oil. UPLC-MS-4: Rt=0.89 min; MS m/z [M-Boc+H]⁺227.3

### Step 2: (2,2-Dimethylpiperidin-4-yl)(3-methoxyazetidin-1-yl)methanone

To tert-butyl 4-(3-methoxyazetidine-1-carbonyl)-2,2-dimethylpiperidine-1-carboxylate (Step 1, 11.1 g, 33.9 mmol) in CH₂Cl₂ (126 mL) was added TFA (39.2 mL, 509 mmol) and the reaction mixture was stirred at RT for 5 h. The mixture was concentrated and the residue was dissolved with dioxane, frozen and lyophilized to give the title compound as a TFA salt. The material was dissolved in MeOH (1000 mL) and MP-carbonate (346 mmol, 110 g) was added. The mixture was swirled at RT for 1 h then was filtered and the filtrate was concentrated under reduced pressure to give the title compound as a light yellow oil. UPLC-MS-4: Rt=0.13 min; MS m/z [M+H]⁺ 227.3.

### Intermediate A83: N,N,2,2-Tetramethylpiperidine-4-carboxamide

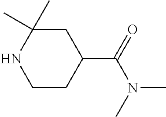

### Step 1: Tert-butyl 4-(dimethylcarbamoyl)-2,2-dimethylpiperidine-1-carboxylate

To a mixture of 1-(tert-butoxycarbonyl)-2,2-dimethylpiperidine-4-carboxylic acid (2.00 g, 7.77 mmol), and dimethylamine (2M in THF, 5.83 mL, 11.7 mmol), in DMA (20 mL) were added HATU (5.91 g, 15.5 mmol) and DIEA (3.33 mL, 19.4 mmol) and the reaction mixture was stirred at 25° C. for 1 h. Water (30 mL) was added and the RM was extracted EtOAc (×2). The combined organic extracts were washed with water, dried (Na₂SO₄), filtered and concentrated. The crude residue was purified by normal phase chromatography (eluent: MeOH in CH₂Cl₂ 0 to 10%) to give the title compound. UPLC-MS-4: Rt=0.12 min; MS m/z [M+H]⁺ 285.3.

### Step 2: N,N,2,2-Tetramethylpiperidine-4-carboxamide

To a solution of tert-butyl 4-(dimethylcarbamoyl)-2,2-dimethylpiperidine-1-carboxylate (315 mg, 1.11 mmol) in dioxane (4 mL) was added HCl (4N in dioxane, 2.77 mL, 11.1 mmol) and the reaction mixture was stirred at RT. After completion of the reaction, the RM was evaporated to give the title compound as a hydrochloride salt which was used without purification in the next step. UPLC-MS-4: Rt=0.12 min; MS m/z [M+H]⁺ 185.3.

### Intermediate A84: (2,2-Dimethylpiperidin-4-yl)(4-methylpiperazin-1-yl)methanone

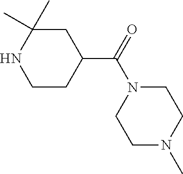

### Step 1: Tert-butyl 2,2-dimethyl-4-(4-methylpiperazine-1-carbonyl)piperidine-1-carboxylate

To a solution of 1-(tert-butoxycarbonyl)-2,2-dimethylpiperidine-4-carboxylic acid (2.50 g, 11.7 mmol) and (N)-methyl piperazine (1.75 g, 17.5 mmol) in DMF (25 mL) at RT were added sequentially EDC·HCl (3.30 g, 17.5 mmol) and HOBt (2.00 g, 15 mmol). The reaction mixture was stirred for 5 min, DIPEA (6.10 mL, 35.0 mmol) was added dropwise and the reaction mixture was stirred at RT for 16 h. The RM was quenched with a sat. aq. NaHCO₃ solution and extracted with EtOAc. The combined organic extracts were washed with brine and dried (Na₂SO₄), filtered and concentrated under vacuum. The crude residue was purified by reverse phase chromatography (eluent: 0 to 50% CH₃CN in water containing 0.1% NH₃) to give the title compound. UPLC-MS-15: Rt=2.72 min, MS m/z [M+H]⁺ 340.2.

### Step 2: (2,2-Dimethylpiperidin-4-yl)(4-methylpiperazin-1-yl)methanone

To a solution of tert-butyl 2,2-dimethyl-4-(4-methylpiperazine-1-carbonyl)piperidine-1-carboxylate (Step 1, 2.60 g, 7.70 mmol) in CH₂Cl₂ (25 mL) was added HCl (4M in dioxane, 10 mL) at 0° C. and the reaction mixture was allowed to reach RT and was stirred for 1 h. The RM was concentrated under vacuum, the crude residue was dissolved in MeOH (10 mL) and tetraalkylammonium carbonate resin (1.50 g) was added. The mixture was stirred for 15 min until reaction pH became basic and the mixture was filtered through a celite bed and the filtrate was concentrated to give the title compound. UPLC-MS-15: Rt=1.35 min, MS m/z [M+H]⁺ 240.2.

### Intermediate A85: (R)-4-(((Tert-butyldiphenylsilyl)oxy)methyl)-2,2-dimethylpiperidine

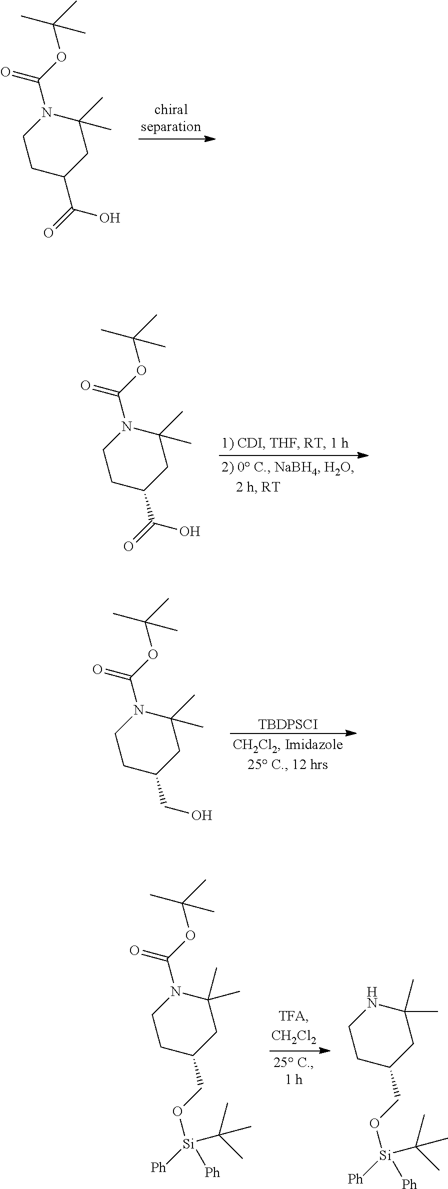 [SMILES: CC(C)(C)OC(=O)N1CCC(C(=O)O)CC1(C)C ; "TBDPSCI" ; CC(C)(C)OC(=O)N1CC[C@@H](C(=O)O)CC1(C)C ; CC(C)(C)OC(=O)N1CC[C@@H](CO)CC1(C)C ; CC(C)(C)OC(=O)N1CC[C@@H](CO[Si](c2ccccc2)(c2ccccc2)C(C)(C)C)CC1(C)C ; CC1(C)C[C@H](CO[Si](c2ccccc2)(c2ccccc2)C(C)(C)C)CCN1]

### Step 1: (R)-1-(Tert-butoxycarbonyl)-2,2-dimethylpiperidine-4-carboxylic acid

The enantiomers of 1-(tert-butoxycarbonyl)-2,2-dimethylpiperidine-4-carboxylic acid (500 g) were separated by chiral SFC to give the title compound as the second eluting enantiomer: C-SFC-43 (mobile phase: CO₂/MeOH 85/20): Rt=3.17 min (note: Rt: 2.38 min for (S)-1-(tert-butoxycarbonyl)-2,2-dimethylpiperidine-4-carboxylic acid); UPLC-MS-4: Rt=0.94 min; MS m/z [M+H]⁻ 256.2.

### Step 2: Tert-butyl (R)-4-(hydroxymethyl)-2,2-dimethylpiperidine-1-carboxylate

To a solution of (R)-1-(tert-butoxycarbonyl)-2,2-dimethylpiperidine-4-carboxylic acid (Step 1, 50.0 g, 194 mmol) in THF (500 mL) was added CDI (37.8 g, 233 mmol) and the mixture was stirred at 25° C. for 1 h. Then a solution of NaBH₄ (13.2 g, 349 mmol) in H₂O (250 mL) was added to the mixture at 0° C. and the reaction mixture was stirred at 25° C. for 16 h. The RM was cooled to 0° C. and then extracted twice with EtOAc. The organic layer was washed with HCl (1N, 200 mL), then washed with NaOH (1N, 200 mL), dried (Na₂SO₄), filtered and concentrated under reduced pressure. The crude residue was purified by normal phase chromatography (eluent: Petroleum ether/EtOAc from 1/0 to 0/1) to give the title compound as a colorless oil. UPLC-MS-4: Rt=0.94 min; MS m/z [M+H-Boc]⁺ 144.3.

### Step 3: Tert-butyl (R)-4-(((tert-butyldiphenylsilyl)oxy)methyl)-2,2-dimethylpiperidine-1-carboxylate

To a solution of tert-butyl (R)-4-(hydroxymethyl)-2,2-dimethylpiperidine-1-carboxylate (Step 2, 130 g, 534 mmol) in CH₂Cl₂ (1.3 L) was added imidazole (72.7 g, 1.07 mol) and TBDPSCI (176 g, 641 mmol). The mixture was stirred at 25° C. for 12 h. After completion of the reaction, the reaction mixture was diluted with CH₂Cl₂ and washed with a sat. aq. NaHCO₃ solution. The combined organic layers were dried (Na₂SO₄), filtered and concentrated under reduced pressure. The crude residue was purified by normal phase chromatography (eluent: Petroleum ether/EtOAc from 1/0 to 0/1) to give the title compound as a colorless oil. LCMS-19: Rt=1.26 min; MS m/z [M+H]⁺ 482.2; [M-Boc+H]⁺382.2.

### Step 4: (R)-4-(((tert-butyldiphenylsilyl)oxy)methyl)-2,2-dimethylpiperidine

To a solution of tert-butyl (R)-4-(((tert-butyldiphenylsilyl)oxy)methyl)-2,2-dimethylpiperidine-1-carboxylate (Step 3, 154 g, 319 mmol) in CH₂Cl₂ (1.54 L) was added TFA (307 mL, 4.16 mol). The reaction mixture was stirred at 25° C. for 1 h and then was concentrated under reduced pressure. The residue was triturated with MBTE (500 mL) and filtered to give a cake. The cake was dissolved in CH₂Cl₂ (500 mL), NaOH (2M, 250 mL) was added and the mixture was stirred at 25° C. for 5 h. The aqueous layer was separated and the organic layer was washed with H₂O (500 mL) then dried (Na₂SO₄), filtered and concentrated under reduced pressure. The crude product was purified by normal phase chromatography (eluent: Petroleum ether/EtOAc from 1/1 to 0/1) to give the title compound as a colorless oil. LCMS-19: Rt=0.853 min; MS m/z [M+H]⁺ 382.2.

### Intermediate A86: 4-((2-(Methoxymethyl)-2-methylpiperidin-4-yl)methyl)morpholine

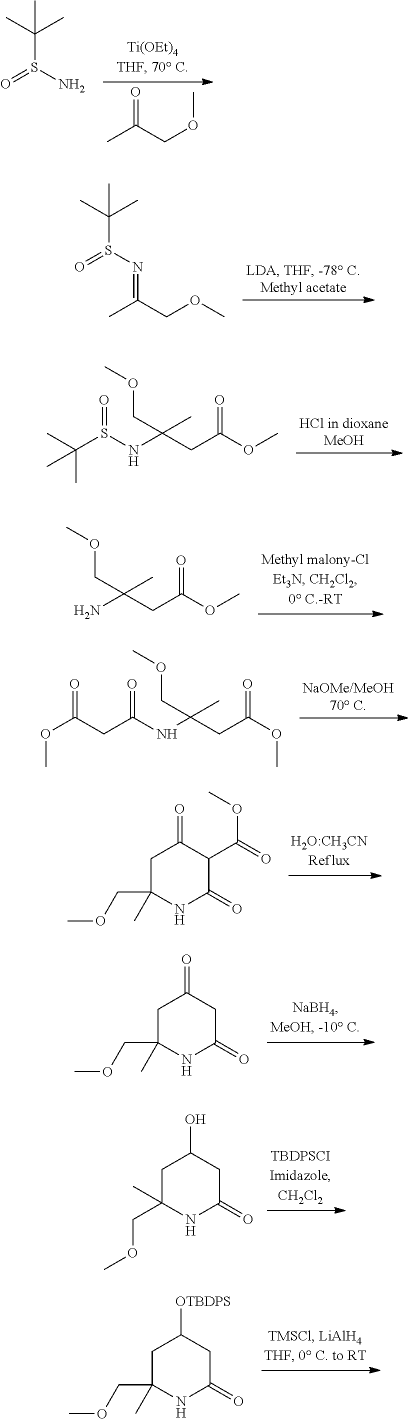

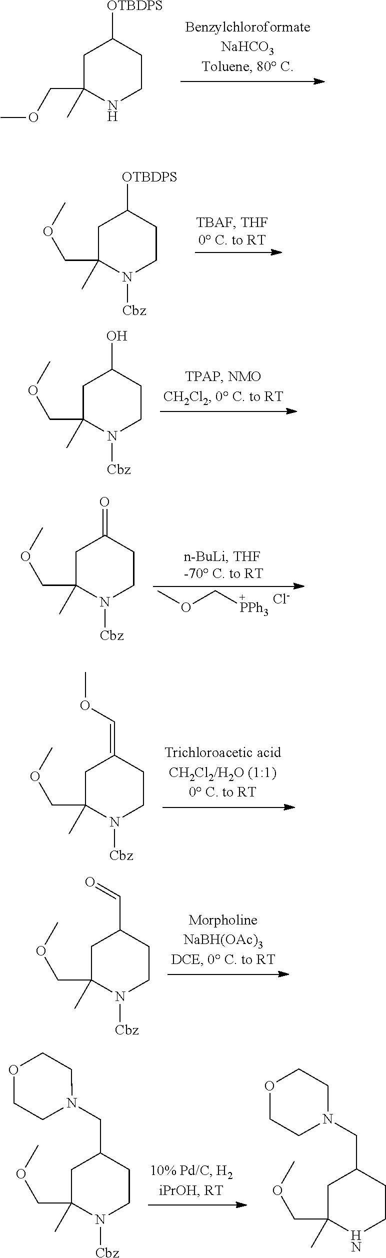

### Step 1: N-(1-Methoxypropan-2-ylidene)-2-methylpropane-2-sulfinamide

To a solution of 2-methylpropane-2-sulfinamide (55.0 g, 453.8 mmol) and 1-methoxypropan-2-one (47.98 g, 544.5 mmol) in THF (500 mL) was added dropwise titanium tetraethoxide (124.2 g, 544.5 mmol) at 0° C. and the reaction mixture was heated to 70° C. and stirred for 16 h. After completion of the reaction, the RM was diluted with EtOAc and poured into a sat. aq. NaHCO₃ solution (800 mL) and stirred for 30 min. The mixture was filtered through a pad of celite and washed with hot EtOAc. The filtrate was dried (Na₂SO₄), filtered and concentrated under reduced pressure. The crude residue was purified by normal phase chromatography (eluent: 25 to 40% EtOAc in hexane) to give the title product. ¹H NMR (400 MHz, CDCl₃) δ 4.07 (s, 2H), 3.43 (s, 3H), 2.37 (s, 3H), 1.29 (s, 9H).

### Step 2: Methyl 3-((tert-butylsulfinyl)amino)-4-methoxy-3-methylbutanoate

To a solution of LDA (2.0 M in THF/hexane, 102 mL, 203.9 mmol) at −78° C. was added dropwise under nitrogen atmosphere a solution of methyl acetate (15.1 g, 204 mmol) in THF (100 mL) and the reaction mixture was allowed to stir at −78° C. for 2 h. Then a solution of N-(1-methoxypropan-2-ylidene)-2-methylpropane-2-sulfinamide (Step 1, 19.5 g, 101.9 mmol) in THF (100 mL) was added dropwise and the RM was stirred for 1 h at −78° C. After completion of the reaction, the RM was quenched by addition at −78° C. of a sat. aq. NH₄Cl solution and the resulting mixture was poured into ice-cold water and extracted with EtOAc. The combined organic extracts were washed with brine, dried (Na₂SO₄), filtered and concentrated under vacuum. The crude residue was purified by normal phase chromatography (eluent: 40 to 60% EtOAc in hexane) to give the title product as an orange Liquid. UPLC-MS-16: Rt=2.51 min, MS m/z [M+H]⁺ 266.

### Step 3: Methyl 3-amino-4-methoxy-3-methylbutanoate

To a solution of methyl 3-((tert-butylsulfinyl)amino)-4-methoxy-3-methylbutanoate (Step 2, 29.0 g, 109.4 mmol) in MeOH (100 mL) at 0° C. was added HCl (2M in MeOH, 60 mL) and the reaction mixture was stirred at RT for 4 h. The solvent was evaporated, co-distilled with CH₂Cl₂ to afford a crude residue which was purified by Dowex resin (eluent: 2% methanolic ammonia in CH₂Cl₂) to give the title product as an orange liquid. UPLC-MS-16: Rt=1.61 min, MS m/z [M+H]⁺ 162.3.

### Step 4: Methyl 4-methoxy-3-(3-methoxy-3-oxopropanamido)-3-methylbutanoate

To a solution of methyl 3-amino-4-methoxy-3-methylbutanoate (Step 3, 14.0 g, 86.9 mmol) in CH₂Cl₂ (140 mL) cooled to 0° C. was added dropwise Et₃N (36.3 g, 261 mmol) followed by the addition dropwise of methyl malonyl chloride (23.6 g, 174 mmol). The reaction mixture was stirred at RT for 4 h. After completion of the reaction, volatils were evaporated and the residue was diluted with water and extracted with CH₂Cl₂. The combined organic extracts were washed with water, brine, dried (Na₂SO₄), filtered and concentrated under vacuum. The crude residue was purified by normal phase chromatography (eluent: 20 to 40% EtOAc in hexane) to give the title product as an orange oil. UPLC-MS-16: Rt=2.02 min, MS m/z [M+H]⁺ 262.2.

### Step 5: Methyl 6-(methoxymethyl)-6-methyl-2,4-dioxopiperidine-3-carboxylate

To a solution of methyl 4-methoxy-3-(3-methoxy-3-oxopropanamido)-3-methylbutanoate (Step 4, 16.0 g, 61.2 mmol) in MeOH (120 mL) at 0° C. was added a solution of NaOMe (25% in MeOH, 16.0 mL, 73.5 mmol) and the reaction mixture was slowly heated to 70° C. and stirred for 15 h. After completion of the reaction, the solvent was evaporated and the residue was diluted with water, acidified with aq. HCl to pH 3 and extracted with 10% iPrOH in CHCl₃. The combined organic extracts were dried (Na₂SO₄), filtered and concentrated under vacuum to give the title product which was used in the next step without purification. UPLC-MS-12: Rt=6.82 min, MS m/z [M+H]⁺ 230.0.

### Step 6: 6-(Methoxymethyl)-6-methylpiperidine-2,4-dione

A solution of methyl 6-(methoxymethyl)-6-methyl-2,4-dioxopiperidine-3-carboxylate (Step 5, 13.5 g, 58.9 mmol) in CH₃CN (containing 1% water, 120 mL) was stirred at 70° C. for 24 h. After completion of the reaction, the solvent was evaporated, co-distilled with toluene and the resulting crude residue was purified by trituration in Et₂O to give the title product as creamy solid. UPLC-MS-12: Rt=4.95 min, MS m/z [M+H]⁺ 172.2.

### Step 7: 4-Hydroxy-6-(methoxymethyl)-6-methylpiperidin-2-one

To a solution of 6-(methoxymethyl)-6-methylpiperidine-2,4-dione (6.60 g, 38.5 mmol) in MeOH (70 mL) at 0° C. was added portion wise NaBH₄ (2.93 g, 77.1 mmol) and the reaction mixture was stirred at 0° C. for 1 h. After completion of the reaction, the RM was quenched by addition of a sat. aq. NH₄Cl solution (10 mL) at 0° C., concentrated under vacuum and co-distilled with MeOH to afford a crude residue. The residue was diluted and stirred in CH₂Cl₂ (containing 5% MeOH) for 20 min and then filtered through a pad of celite. The filtrate was dried (Na₂SO₄), filtered and concentrated under vacuum to give the title product as a white solid. UPLC-MS-16: Rt=1.27 min, MS m/z [M+H]⁺ 174.0.

### Step 8: 4-((Tert-butyldiphenylsilyl)oxy)-6-(methoxymethyl)-6-methylpiperidin-2-one

To a solution of 4-hydroxy-6-(methoxymethyl)-6-methylpiperidin-2-one (Step 7, 6.40 g, 36.9 mmol) in CH₂Cl₂ (150 mL) was added at 0° C. imidazole (7.54 g, 111 mmol) followed by portion wise addition of TBDPS-Chloride (17.2 g, 62.8 mmol). The reaction mixture was stirred at RT for 16 h. The white solid was filtered, washed with CH₂Cl₂ and the filtrate was washed with water, brine, dried (Na₂SO₄), filtered and concentrated. The crude residue was purified by normal phase chromatography (eluent: 20 to 50% EtOAc in hexane) to give the title product. UPLC-MS-9: Rt=1.59 min, [M+H]⁺=412.4.

### Step 9: 4-((Tert-butyldiphenylsilyl)oxy)-2-(methoxymethyl)-2-methylpiperidine

To a solution of 4-((tert-butyldiphenylsilyl)oxy)-6-(methoxymethyl)-6-methylpiperidin-2-one (Step 8, 15.0 g, 36.4 mmol) in THF (150 mL) was added dropwise at −10° C. TMS-Chloride (18.6 g, 109.3 mmol) and reaction mixture was stirred for 1 h at −10° C. under a nitrogen atmosphere. Then, LiALH₄ (1M in THF, 145 mL, 145 mmol) was added dropwise and the reaction mixture was stirred at −10° C. for 8 h. The RM was quenched by addition of aq. NaOH (2N) solution at 0° C. and extracted with EtOAc. The combined organic layers were dried (Na₂SO₄), filtered and concentrated under vacuum to give the title product, which was used in the next step without purification. UPLC-MS-6: Rt=1.71 min, MS m/z [M+H]⁺ 397.7.

### Step 10: Benzyl 4-((tert-butyldiphenylsilyl)oxy)-2-(methoxymethyl)-2-methylpiperidine-1-carboxylate

To a solution of 4-((tert-butyldiphenylsilyl)oxy)-2-(methoxymethyl)-2-methylpiperidine (Step 9, 12.0 g, 30.2 mmol) in toluene (120 mL) was added NaHCO₃ (8.87 g, 105.6 mmol) followed by the dropwise addition of benzylchloroformate (50% in toluene, 25.7 g, 75.5 mmol). The reaction mixture was stirred at 80° C. for 3 h. After completion of the reaction, the RM was poured into ice-water and extracted with EtOAc. The combined organic layers were washed with a sat. aq. NaHCO₃ solution, brine, dried (Na₂SO₄), filtered and concentrated under vacuum. The crude residue was purified by normal phase chromatography (eluent: 0 to 2% EtOAc in hexane) to give the title product as an orange oil. UPLC-MS-13: Rt=3.14 & 3.22 min, MS m/z [M+H]⁺ 533.4.

### Step 11: Benzyl 4-hydroxy-2-(methoxymethyl)-2-methylpiperidine-1-carboxylate

To a solution of benzyl 4-((tert-butyldiphenylsilyl)oxy)-2-(methoxymethyl)-2-methylpiperidine-1-carboxylate (Step 10, 11.5 g, 22.3 mmol) in THF (150 mL) was added at 0° C. TBAF (1M in THF, 44.6 mL, 44.6 mmol) dropwise and the reaction mixture was stirred at RT for 8 h. After completion of the reaction, the RM was poured into ice-water and extracted with EtOAc. The combined organic layers were washed with water, brine, dried (Na₂SO₄), filtered and concentrated under vacuum to afford the title product which was used in the next step without purification. UPLC-MS-5: Rt=1.61 min & 1.63 min, MS m/z [M+H]⁺ 294.1.

### Step 12: Benzyl 2-(methoxymethyl)-2-methyl-4-oxopiperidine-1-carboxylate

To a solution of benzyl 4-hydroxy-2-(methoxymethyl)-2-methylpiperidine-1-carboxylate (Step 11, 6.00 g, 20.4 mmol) in CH₂Cl₂ (60 mL) was added molecular sieves. The mixture was cooled to 0° C. and N-methyl morpholine oxide (3.60 g, 30.7 mmol) was added followed by the portion wise addition of tetrapropylammonium perruthenate (0.35 g, 1.02 mmol) and the reaction mixture was stirred at RT for 15 min. After completion of the reaction, the RM was poured into ice-water and extracted with CH₂Cl₂. The combined organic layers were washed with water, brine, dried (Na₂SO₄), filtered and concentrated under vacuum. The crude residue was purified by normal phase chromatography (eluent: 15 to 25% EtOAc in hexane) to give the title product as an orange oil. UPLC-MS-9: Rt=1.25 min, [M+H]⁺ 292.4.

### Step 13: Benzyl-2-(methoxymethyl)-4-(methoxymethylene)-2-methylpiperidine-1-carboxylate

To a suspension of (methoxymethyl)triphenylphosphonium chloride (15.9 g, 46.3 mmol) in THF (50 mL) at −78° C. was added dropwise under a nitrogen atmosphere n-BuLi (2.5M in hexane, 21.7 mL, 54.1 mmol) and the mixture was stirred at −78° C. for 1 h. A solution of benzyl 2-(methoxymethyl)-2-methyl-4-oxopiperidine-1-carboxylate (Step 12, 4.50 g, 15.4 mmol) in THF (5 mL) was then added dropwise at −78° C. and the reaction mixture was allowed to reach RT and was stirred for 16 h. After completion of the reaction, the RM was poured onto ice-water and extracted with EtOAc. The combined organic layers were washed with water, brine, dried (Na₂SO₄), filtered and concentrated under vacuum. The crude residue was purified by normal phase chromatography (eluent: 6 to 8% EtOAc in hexane) to give the title product as an orange oil. UPLC-MS-9: Rt=1.43 min, [M+H]⁺ 320.2.

### Step 14: Benzyl 4-formyl-2-(methoxymethyl)-2-methylpiperidine-1-carboxylate

To a solution of benzyl-2-(methoxymethyl)-4-(methoxymethylene)-2-methylpiperidine-1-carboxylate (Step 13, 1.60 g, 5.0 mmol) in CH₂Cl₂/Water (2/1, 30 mL) was added trichloroacetic acid (8.20 g, 50.2 mmol) and reaction mixture was stirred at RT for 16 h. After completion of the reaction, the RM was poured into ice-water, neutralized at 0° C. with a sat. aq. NaHCO₃ solution and extracted with EtOAc. The combined organic layers were washed with a sat. aq. NaHCO₃ solution, brine, dried (Na₂SO₄), filtered and concentrated under vacuum. The crude residue was purified by normal phase chromatography (eluent: 8 to 15% EtOAc in hexane) to give the title product as an orange oil. UPLC-MS-16: Rt=3.39 min, 3.46 min, MS m/z [M+H]⁺ 306.0.

### Step 15: Benzyl 2-(methoxymethyl)-2-methyl-4-(morpholinomethyl)piperidine-1-carboxylate

To a solution of benzyl 4-formyl-2-(methoxymethyl)-2-methylpiperidine-1-carboxylate (Step 14, 1.00 g, 3.28 mmol) in dichloroethane (10 mL) was added morpholine (0.37 g, 4.26 mmol) and mixture was allowed to stirr at RT for 2 h. The mixture was cooled to 0° C., NaBH(OAc)₃ (1.73 g, 8.18 mmol) was added and the reaction mixture was stirred at RT for 6 h. After completion of the reaction, the RM was poured into ice-water, neutralized by addition of a sat. aq. NaHCO₃ solution and extracted with EtOAc. The combined organic layers were washed with brine, dried (Na₂SO₄), filtered and concentrated under vacuum. The crude residue was purified by reverse phase chromatography (eluent: 55% CH₃CN in water containing 0.1% NH₃) to give the title product. UPLC-MS-16: Rt=3.52 min & 3.63 min, MS m/z [M+H]⁺ 377.3.

### Step 16: 4-((2-(Methoxymethyl)-2-methylpiperidin-4-yl)methyl)morpholine

To a solution of benzyl 2-(methoxymethyl)-2-methyl-4-(morpholinomethyl)piperidine-1-carboxylate (Step 15, 1.10 g, 2.92 mmol) in iPrOH (11 mL) was added 10% Pd/C (0.3 g) and the reaction mixture was stirred at RT for 6 h under hydrogen atmosphere. The reaction mixture was filtered through a celite bed, washed with excess of EtOAc and the filtrate was concentrated under vacuum and co-distilled with toluene to give the title product. UPLC-MS-16: Rt=1.55 min, [M+H]⁺ 243.3.

### Intermediate A87: 4-((2-Ethyl-2-methylpiperidin-4-yl)methyl)morpholine

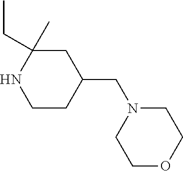 [SMILES: CCC1(C)CC(CN2CCOCC2)CCN1]

The title compound was prepared in 16 steps by a method similar to that of (4-((2-(methoxymethyl)-2-methylpiperidin-4-yl)methyl)morpholine (Intermediate A86) using butan-2-one (Step 1) instead of 1-methoxypropan-2-one. UPLC-MS-16: Rt=1.63 min, MS m/z [M+H]⁺ 227.3.

### Intermediate A88: 4-((2,2-Dimethyl-1,2,3,6-tetrahydropyridin-4-yl)methyl)morpholine

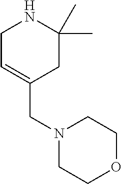

### Step 1: Tert-butyl 2,2-dimethyl-4-(((trifluoromethyl)sulfonyl)oxy)-3,6-dihydropyridine-1(2H)-carboxylate

To a solution of tert-butyl 2,2-dimethyl-4-oxopiperidine-1-carboxylate (1.50 g, 6.60 mmol) in THF (10 mL) under Argon at −78° C. was added LiHMDS (1M in THF, 6.60 mL, 6.60 mmol) under inert atmosphere and the reaction mixture was stirred at −78° C. for 20 min. Then a solution of 1,1,1-trifluoro-N-phenyl-N-((trifluoromethyl)sulfonyl)methanesulfonamide (2.36 g, 6.60 mmol) in THF (10 mL) was added at −78° C., and the RM was slowly warmed to RT and stirred for 16 h. The RM was quenched by addition of a sat. aq. NH₄Cl solution and extracted with EtOAc. The combined organic extracts were washed with brine, dried (MgSO₄), filtered and concentrated under vacuum. The crude residue was purified by normal phase chromatography (eluent: EtOAc in n-heptane 0 to 100%) to give the title compound. UPLC-MS-4: Rt=1.44 min; MS m/z [M+H-Boc]⁺260.1.

### Step 2: Tert-butyl 2,2-dimethyl-4-(morpholinomethyl)-3,6-dihydropyridine-1(2H)-carboxylate

Tert-butyl 2,2-dimethyl-4-(((trifluoromethyl)sulfonyl)oxy)-3,6-dihydropyridine-1(2H)-carboxylate (Step 1, 1.28 g, 3.56 mmol), potassium trifluoro(morpholinomethyl)borate (1.11 g, 5.34 mmol), X-Phos (0.34 g, 0.71 mmol), Pd(OAc)₂ (0.08 g, 0.36 mmol) and Cs₂CO₃ (3.48 g, 10.7 mmol) were suspended in dry dioxane (16 mL) under Argon. Then water (1.78 mL) was added and the reaction mixture was heated at 80° C. for 2.5 h. The reaction was quenched by addition of a sat. aq. NaHCO₃ solution and extracted with EtOAc. The combined organic extracts were washed with brine and dried (MgSO₄), filtered and concentrated under vacuum. The crude residue was purified by reverse phase chromatography (mobile phase: 0 to 70% [H₂O containing 0.1% TFA] in CH₃CN); to give the title compound. UPLC-MS-4: Rt=0.52 min; MS m/z [M+H]⁺ 311.4.

### Step 3: 4-((2,2-Dimethyl-1,2,3,6-tetrahydropyridin-4-yl)methyl)morpholine

To a solution of tert-butyl 2,2-dimethyl-4-(morpholinomethyl)-3,6-dihydropyridine-1(2H)-carboxylate (Step 2, 600 mg, 1.93 mmol) in dioxane (9.65 mL) was added TFA (298 μl, 3.87 mmol) at RT and the reaction mixture was stirred at RT for 16 h. The mixture was evaporated to dryness, the crude residue was dissolved in methanol (10 mL), MP-carbonate (2.56 g, 7.73 mmol) was added and the mixture was swirled at 40° C. for 1 h. The mixture was filtered, the filtrate was concentrated under vacuum and the crude residue was purified by normal phase chromatography (basic alumina column, eluent: [CH₂Cl₂/MeOH: 80/20] in CH₂Cl₂ from 0 to 100%) to give the title compound. UPLC-MS-4: Rt=0.13 min; MS m/z [M+H]⁺ 211.3.

### Intermediate A89: 5,7-Dimethyl-2,5,7-triazaspiro[3.4]octan-6-one

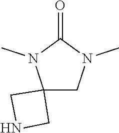

### Step 1: Tert-butyl 5-methyl-6-oxo-2,5,7-triazaspiro[3.4]octane-2-carboxylate

Di-tert-butyl dicarbonate (1.44 mL, 6.19 mmol) and triethylamine (2.35 mL, 16.9 mmol) were added to a solution of 5-methyl-2,5,7-triazaspiro[3.4]octan-6-one (1.00 g, 5.63 mmol) in THF (50 mL) and the reaction mixture was stirred at RT for 1 h. The reaction mixture was diluted with CH₂Cl₂, washed with a sat. aq. NaHCO₃ solution and with brine. The organic layer was dried (Na₂SO₄), filtered and concentrated in vacuum. The crude residue was purified by normal phase chromatography (eluent: [CH₂Cl₂/MeOH: 80/20] in CH₂Cl₂ from 1 to 100%) to give the title compound. UPLC-MS-4: Rt=0.54 min; MS m/z [M+H]⁺ 242.2.

### Step 2: Tert-butyl 5,7-dimethyl-6-oxo-2,5,7-triazaspiro[3.4]octane-2-carboxylate

To a solution of tert-butyl 5-methyl-6-oxo-2,5,7-triazaspiro[3.4]octane-2-carboxylate (1.24 g, 5.15 mmol) in DMF (50 mL) at 0° C. under inert atmosphere, was added sodium hydride (50% in mineral oil, 412 mg, 10.3 mmol) and the reaction mixture was stirred at 0° C. for 10 min. Then methyl iodide (0.42 mL, 6.69 mmol) was added. After stirring for 1 h at 0° C., the reaction was complete. The RM was quenched with water, diluted with dichloromethane and washed with a sat. aq. NaHCO₃ solution, water and brine. The organic layer was dried (Na₂SO₄), filtered and concentrated in vacuum. The crude residue was purified by normal phase chromatography (eluent: [CH₂Cl₂/MeOH: 80/20] in CH₂Cl₂ from 5 to 100%) to give the title compound. UPLC-MS-4: Rt=0.62 min; MS m/z [M+H]⁺ 256.2.

### Step 3: 5,7-Dimethyl-2,5,7-triazaspiro[3.4]octan-6-one

To a solution of tert-butyl 5,7-dimethyl-6-oxo-2,5,7-triazaspiro[3.4]octane-2-carboxylate (Step 2, 2.06 g, 8.07 mmol) in CH₂Cl₂ (20 mL) was added TFA (19.4 mL, 242 mmol) and the solution was stirred at RT for 15 min. The RM was evaporated to dryness, the crude was dissolved in methanol (40 mL), MP-Carbonate (7.0 g, 22.6 mmol) was added and the mixture was swirled at 40° C. for 1 h, then was filtered and the filtrate was concentrated under vacuum to give the title compound. UPLC-MS-4: Rt=0.12 min; MS m/z [M+H]⁺ 156.2.

### Intermediate A90: 6-(Methylsulfonyl)-2,6-diazaspiro[3.4]octane

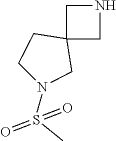

### Step 1: Tert-butyl 6-(methylsulfonyl)-2,6-diazaspiro[3.4]octane-2-carboxylate

To a solution of tert-Butyl 2,6-diazaspiro[3.4]octane-2-carboxylate (1.00 g, 4.71 mmol) in CH₂Cl₂ (47 mL) was added triethylamine (1.38 mL, 9.89 mmol). The solution was cooled to 0° C., methanesulfonyl chloride (0.40 mL, 5.18 mmol) was added dropwise at 0° C. and the reaction was allowed to reach RT and stirred at RT for 2.5 h. The RM was quenched with water and extracted with CH₂Cl₂. The organic layer was dried (Na₂SO₄), filtered and concentrated in vacuum. The crude residue was purified by normal phase chromatography (eluent: [CH₂Cl₂/MeOH: 80/20] in CH₂Cl₂ from 1 to 30%) to give the title compound. UPLC-MS-4: Rt=0.62 min; MS m/z [M+H-Boc]⁺191.1.

### Step 2: 6-(Methylsulfonyl)-2,6-diazaspiro[3.4]octane

To a solution of tert-butyl 6-(methylsulfonyl)-2,6-diazaspiro[3.4]octane-2-carboxylate (Step 1, 1.50 g, 4.65 mmol) in CH₂Cl₂ (46 mL) was added TFA (11.2 mL, 139 mmol) and the solution was stirred at RT for 15 min. The RM was evaporated to dryness. The crude residue was suspended in diethylether (20 mL) and the solid was washed with diethylether and dried under vacuum. The residue was purified by normal phase chromatography (alumina column, eluent: [CH₂Cl₂/MeOH: 80/20] in CH₂Cl₂ from 5 to 100%) to give the title compound. UPLC-MS-4: Rt=0.13 min; MS m/z [M+H]⁺ 191.2.

### Intermediate B1: 8 (S)-(1,4-Dioxan-2-yl)methyl 4-methylbenzenesulfonate

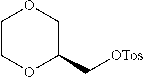

To a solution of (R)-(1,4-dioxan-2-yl)methanol (87.0 mg, 0.70 mmol) in CH₂Cl₂ (3 mL) at 0° C. was added under argon triethylamine (0.20 mL, 1.40 mmol) followed by toluene-4-sulfonyl chloride (160 mg, 0.84 mmol) and the reaction mixture was stirred at RT for 16 h. The reaction mixture was quenched by addition of a sat. aq. NaHCO₃ solution and extracted with EtOAc. The organic layer was washed with brine, dried (Na₂SO₄), filtered and evaporated to give the title compound which was used in the next step without purification. UPLC-MS-3: Rt=0.66 min; MS m/z [M+H]⁺ 273.2.

### Intermediate B2: (R)-(1,4-Dioxan-2-yl)methyl 4-methylbenzenesulfonate

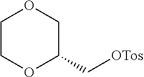

The title compound was prepared by a method similar to that of Intermediate B1 by replacing (R)-(1,4-dioxan-2-yl)methanol with (S)-(1,4-dioxan-2-yl)methanol (CAS [406913-93-7]). UPLC-MS-4: Rt=0.69 min; MS m/z [M+H]⁺ 273.1.

### Intermediate B3: (1,4-Dioxepan-6-yl)methyl 4-methylbenzenesulfonate

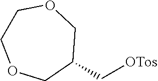

The title compound was prepared by a method similar to that of Intermediate B1 by replacing (R)-(1,4-dioxan-2-yl)methanol with (1,4-dioxepan-6-yl)methanol (CAS [1010836-47-1]). UPLC-MS-4: Rt=0.71 min; MS m/z [M+H]⁺ 287.2.

### Intermediate B4: 3-((Phenylsulfonyl)methylene)oxetane

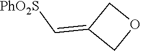

Methyl phenyl sulfone (1.00 g, 6.27 mmol) was dissolved in THF (8 mL) under Argon and cooled to 0° C. n-Butyllithium (1.6M in hexane, 8.20 mL, 13.2 mmol) was added dropwise and the reaction mixture was stirred for 30 min at 0° C. Diethyl chlorophosphate (1.40 mL, 9.41 mmol) was then added and the mixture was stirred for 30 min at 0° C. The RM was then cooled down to −78° C. and oxetan-3-one (0.42 mL, 6.27 mmol) was added. The RM was slowly allowed to reach RT and stirred for 1 h. The RM was quenched with water, diluted with a sat. aq. NaHCO₃ solution and extracted with EtOAc (×2). The combined organic extracts were washed with brine, dried (Na₂SO₄), filtered and evaporated. The crude residue was purified normal phase chromatography (eluent: EtOAc in Heptane 0 to 60%). UPLC-MS-4: Rt=0.42 min; MS m/z [M+H]⁺ 211.1.

### Intermediate B5: (R)-Tetrahydrofuran-2-carbaldehyde

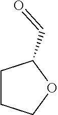

To a stirred solution of Dess-Martin Periodinane (623 mg, 1.47 mmol) and NaHCO₃ (247 mg, 2.94 mmol) in CH₂Cl₂ (5 mL) was added under Argon (R)-tetrahydrofurfuryl alcohol (0.14 mL, 1.47 mmol) at 0° C. and the reaction mixture was stirred at RT for 1 h. The reaction mixture was filtered over celite and concentrated (water bath: 20° C.) to give the title compound which was used in the next step without purification. MS-1: MS m/z [M+H]⁺ 101.2.

### Intermediate B6: 1-((Tetrahydro-2H-pyran-2-yl)oxy)cyclopropane-1-carbaldehyde

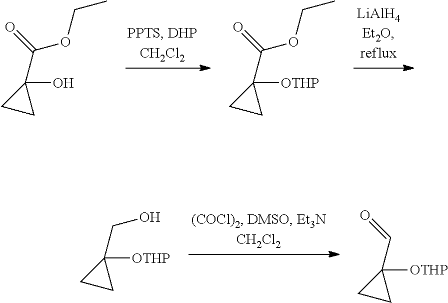

### Step 1: Ethyl 1-((tetrahydro-2H-pyran-2-yl)oxy)cyclopropane-1-carboxylate

To a solution of ethyl 1-hydroxycyclopropane-1-carboxylate (1.00 g, 6.92 mmol) and DHP (0.63 mL, 6.92 mmol) in CH₂Cl₂ (13 mL) was added pyridine 4-methylbenzenesulfonate (174 mg, 0.69 mmol) and the reaction mixture was stirred at RT for 3 h. The reaction mixture was evaporated and the residue was partitioned between Et₂O and brine. The organic layer was dried (Na₂SO₄), filtered and concentrated in vacuo to give the title compound as a yellow oil. MS-1: MS m/z [M+H]⁺ 215.2.

### Step 2: (1-((Tetrahydro-2H-pyran-2-yl)oxy)cyclopropyl)methanol

To a solution of ethyl 1-((tetrahydro-2H-pyran-2-yl)oxy)cyclopropane-1-carboxylate (Step 1, 1.58 g, 7.01 mmol) in Et₂O (35 mL) under a nitrogen atmosphere was added LiAlH₄ (1M in Et₂O, 7.01 mL, 7.01 mmol) at RT and the reaction mixture was refluxed for 1 h. The reaction mixture was cooled to RT and was quenched carefully with ice. The organic layer was separated and washed with water (×2), dried (Na₂SO₄), filtered and concentrated in vacuo to give the title compound as a yellow oil. MS-1: MS m/z [M+H]⁺ 173.2.

### Step 3: 1-((Tetrahydro-2H-pyran-2-yl)oxy)cyclopropane-1-carbaldehyde

To a solution of oxalyl dichloride (0.38 mL, 4.35 mmol) in CH₂Cl₂ (18 mL) was added a solution of DMSO (1.44 mL. 20.3 mmol) in CH₂Cl₂ (3 mL) dropwise at −60° C. After 10 min, a solution of (1-((tetrahydro-2H-pyran-2-yl)oxy)cyclopropyl)methanol (Step 2, 500 mg, 2.90 mmol) in CH₂Cl₂ (10 mL) was added and the reaction mixture was stirred for 30 min. Then Et₃N (2.02 mL, 14.5 mmol) was added and the reaction mixture was stirred for 2 h while slowly warming up to RT over time. The RM was diluted with CH₂Cl₂ and washed with water then brine. The organic layer was dried (Na₂SO₄), filtered and concentrated in vacuo to give the title compound as a yellow oil. ¹H NMR (600 MHz, DMSO-d₆) δ 9.42 (s, 1H), 4.69 (m, 1H), 3.86 (m, 1H), 3.44 (m, 1H), 1.77-1.65 (m, 2H), 1.55-1.43 (m, 4H), 1.40 (m, 1H), 1.33-1.27 (m, 2H), 1.20 (m, 1H).

### Intermediate C1: tert-butyl 6-(3-bromo-5-methyl-1H-pyrazol-1-yl)-2-azaspiro[3.3]heptane-2-carboxylate

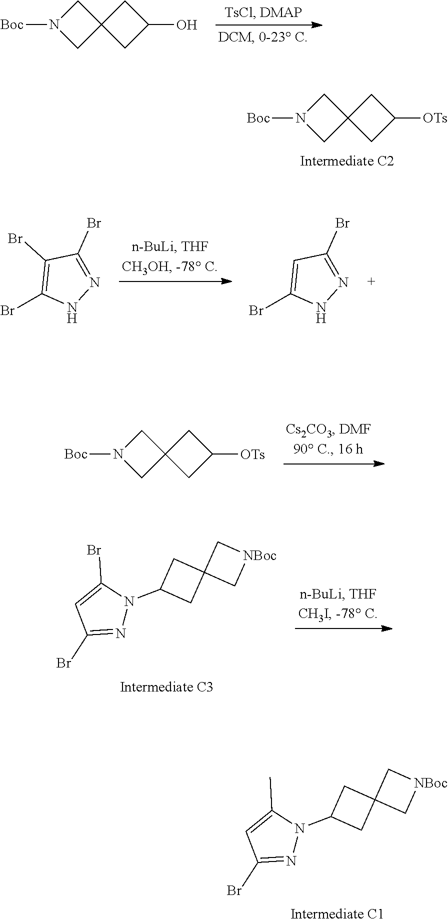

### Step 1: Intermediate C2: Tert-butyl 6-(tosyloxy)-2-azaspiro[3.3]heptane-2-carboxylate

To a solution of tert-butyl 6-hydroxy-2-azaspiro[3.3]heptane-2-carboxylate (CAS [1147557-97-8], 2.92 kg, 12.9 mmol) in CH₂Cl₂ (16.5 L) were added DMAP (316.1 g, 2.59 mol) and Tosyl-Cl (2.96 kg, 15.5 mol) at 20° C.-25° C. To the reaction mixture was added dropwise Et₃N (2.62 kg, 25.9 mol) at 10° C.-20° C. The reaction mixture was stirred 0.5 h at 5° C.-15° C. and then was stirred 1.5 h at RT. After completion of the reaction, the reaction mixture was concentrated under vacuum. To the residue was added NaCl (5% in water, 23 L) followed by extraction with EtOAc (23 L). The combined aqueous layers were extracted with EtOAc (10 L×2). The combined organic layers were washed with NaHCO₃ (3% in water, 10 L×2)) and concentrated under vacuum to give the title compound. ¹H NMR (400 MHz, DMSO-d₆) δ 7.81-7.70 (m, 2H), 7.53-7.36 (m, 2H), 4.79-4.62 (m, 1H), 3.84-3.68 (m, 4H), 2.46-2.38 (m, 5H), 2.26-2.16 (m, 2H), 1.33 (s, 9H). UPLC-MS-1a: Rt=1.18 min; MS m/z [M+H]⁺ 368.2.

### Step 2: 3,5-Dibromo-1H-pyrazole

To a solution of 3,4,5-tribromo-1H-pyrazole (55.0 g, 182.2 mmol) in anhydrous THF (550 mL) was added at −78° C. n-BuLi (145.8 mL, 364.5 mmol) dropwise over 20 min maintaining the internal temperature at −78° C./−60° C. The RM was stirred at this temperature for 45 min. Then the reaction mixture was carefully quenched with MeOH (109 mL) at −78° C. and stirred at this temperature for 30 min. The mixture was allowed to reach to 0° C. and stirred for 1 h. Then, the mixture was diluted with EtOAc (750 mL) and HCl (0.5 N, 300 mL) was added. The layers were separated and the organic layer was washed with brine (350 mL), dried (Na₂SO₄), filtered, and concentrated under vacuum. The crude residue was dissolved in CH₂Cl₂ (100 mL), cooled to −50° C. and petroleum ether (400 mL) was added. The precipitated solid was filtered and washed with n-hexane (250 mL×2) and dried under vacuum to give the title compound. ¹H NMR (400 MHz, DMSO-d₆) δ 13.5 (br s, 1H), 6.58 (s, 1H).

### Step 3: Intermediate C3: Tert-butyl 6-(3,5-dibromo-1H-pyrazol-1-yl)-2-azaspiro[3.3]heptane-2-carboxylate

To a solution of tert-butyl 6-(tosyloxy)-2-azaspiro[3.3]heptane-2-carboxylate (Step 1, 900 g, 2.40 mol) in DMF (10.8 L) was added Cs₂CO₃ (1988 g, 6.10 mol) and 3,5-dibromo-1H-pyrazole (Step 2, 606 g, 2.68 mol) at 15° C. The reaction mixture was stirred at 90° C. for 16 h. The reaction mixture was poured into ice-water/brine (80 L) and extracted with EtOAc (20 L). The aqueous layer was re-extracted with EtOAc (10 L×2). The combined organic layers were washed with brine (10 L), dried (Na₂SO₄), filtered, and concentrated under vacuum. The residue was triturated with dioxane (1.8 L) and dissolved at 60° C. To the light yellow solution was slowly added water (2.2 L), and recrystallization started after addition of 900 mL of water. The resulting suspension was cooled down to 0° C., filtered, and washed with cold water. The filtered cake was triturated with n-heptane, filtered, then dried under vacuum at 40° C. to give the title compound. ¹H NMR (400 MHz, DMSO-d₆) δ 6.66 (s, 1H), 4.86-4.82 (m, 1H), 3.96-3.85 (m, 4H), 2.69-2.62 (m, 4H), 1.37 (s, 9H); UPLC-MS-2a: Rt=1.19 min; MS m/z [M+H]⁺ 420.0/422.0/424.0.

### Step 4: Intermediate C1: Tert-butyl 6-(3-bromo-5-methyl-1H-pyrazol-1-yl)-2-azaspiro[3.3]heptane-2-carboxylate

To a solution of tert-butyl 6-(3,5-dibromo-1H-pyrazol-1-yl)-2-azaspiro[3.3]heptane-2-carboxylate (Intermediate C3, 960 g, 2.30 mol) in THF (9.60 L) was added n-BuLi (1.2 L, 2.50 mol) dropwise at −80° C. under an inert atmosphere. The reaction mixture was stirred 10 min at −80° C. To the reaction mixture was then added dropwise iodomethane (1633 g, 11.5 mol) at −80° C. After stirring for 5 min at −80° C., the reaction mixture was allowed to warm up to 18° C. The reaction mixture was poured into a sat. aq. NH₄Cl solution (4 L) and extracted with CH₂Cl₂ (10 L). The separated aqueous layer was re-extracted with CH₂Cl₂ (5 L) and the combined organic layers were concentrated under vacuum. The crude product was dissolved in 1,4-dioxane (4.8 L) at 60° C., then water (8.00 L) was added dropwise slowly. The resulting suspension was cooled to 17° C. and stirred for 30 min. The solid was filtered, washed with water, and dried under vacuum to give the title compound. ¹H NMR (400 MHz, DMSO-d₆) δ 6.14 (s, 1H), 4.74-4.66 (m, 1H), 3.95-3.84 (m, 4H), 2.61-2.58 (m, 4H), 2.20 (s, 3H), 1.37 (s, 9H); UPLC-MS-1a: Rt=1.18 min; MS m/z [M+H]⁺ 356.1/358.1.

### Intermediate C4: Tert-butyl 6-(3-bromo-1H-pyrazol-1-yl)-2-azaspiro[3.3]heptane-2-carboxylate

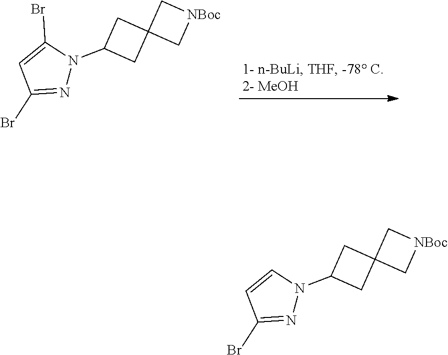 [SMILES: CC(C)(C)OC(=O)N1CC2(CC(n3nc(Br)cc3Br)C2)C1 ; CC(C)(C)OC(=O)N1CC2(CC(n3ccc(Br)n3)C2)C1]

To a stirred solution of tert-butyl 6-(3,5-dibromo-1H-pyrazol-1-yl)-2-azaspiro[3.3]heptane-2-carboxylate (Intermediate C3, 3.80 g, 80.0 mmol) at −78° C. in THF (500 mL) under Ar was added n-BuLi (1.6 M in hexane, 50.2 mL, 80.0 mmol). After 10 min at −78° C., the reaction mixture was quenched by addition of MeOH (30 mL), then was diluted with a sat. aq. NH₄Cl solution and extracted with EtOAc (×2). The combined organic extracts were with a sat. aq. NaHCO₃ solution, dried (Na₂SO₄), filtered and evaporated. The crude residue was purified by normal phase chromatography (eluent: EtOAc in c-hexane from 0 to 50%) to afford the title compound as a white solid. UPLC-MS-4: Rt=1.09 min; MS m/z [M+H]⁺ 342.1/344.1.

### Intermediate C5: Tert-butyl 6-(4-bromo-3-iodo-5-methyl-1H-pyrazol-1-yl)-2-azaspiro[3.3]heptane-2-carboxylate

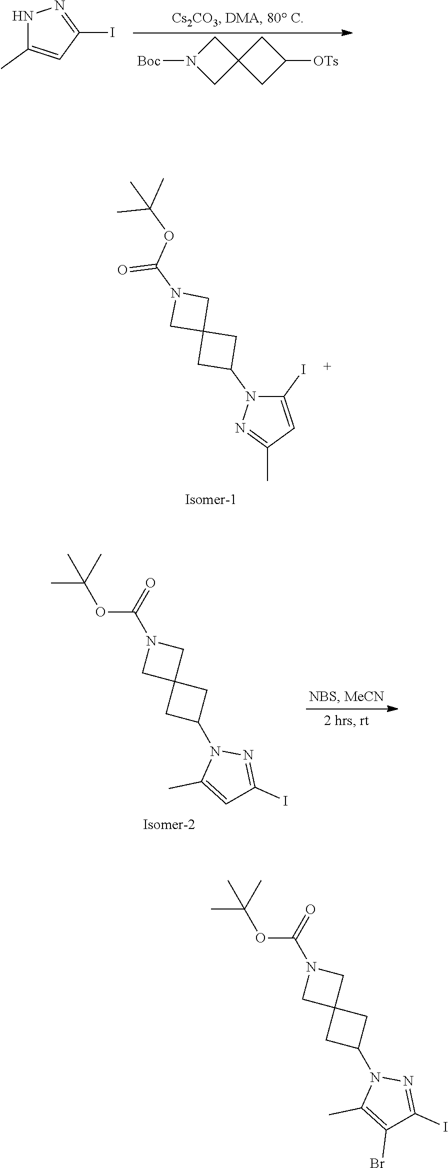

### Step 1: Tert-butyl 6-(3-iodo-5-methyl-1H-pyrazol-1-yl)-2-azaspiro[3.3]heptane-2-carboxylate

To a solution of 3-iodo-5-methyl-1H-pyrazole (340 g, 925 mmol) in DMA (3.4 L) was added Cs₂CO₃ (754 g, 2.31 mol) followed by tert-butyl 6-(tosyloxy)-2-azaspiro[3.3]heptane-2-carboxylate (Intermediate C2, 193 g, 925 mmol). The reaction mixture was stirred at 80° C. for 16 h. The reaction mixture was poured into water (3000 mL) and extracted with EtOAc (1000 mL×3). The combined organic layers were washed with brine (1 L×3), dried (Na₂SO₄), filtered and concentrated under vacuum to give a crude product as mixture of 2 regioisomers. The regioisomers were separated by normal phase chromatography (eluent: heptane/EtOAc from 8/1 to 5/1) to give isomer-1 as a white solid: UPLC-MS-1a: Rt=1.23 min; MS m/z [M+H]⁺ 404.1, and the title compound isomer-2 as a white solid. ¹H NMR (400 MHz, DMSO-d₆) δ 6.24-6.22 (m, 1H), 4.82-4.60 (m, 1H), 4.01-3.92 (m, 2H), 3.88-3.81 (m, 2H), 2.66-2.57 (m, 4H), 2.18 (s, 3H), 1.37 (s, 9H). UPLC-MS-1a: Rt=1.20 min; MS m/z [M+H]⁺ 404.1.

### Step 2: Tert-butyl 6-(4-bromo-3-iodo-5-methyl-1H-pyrazol-1-yl)-2-azaspiro[3.3]heptane-2-carboxylate

To a solution of tert-butyl 6-(3-iodo-5-methyl-1H-pyrazol-1-yl)-2-azaspiro[3.3]heptane-2-carboxylate (Step 1, 100 g, 248 mmol) in acetonitrile (1 L) was added NBS (53.0 g, 298 mmol) and the reaction mixture was stirred at RT for 3 h. The RM was diluted with EtOAc (1.5 L), washed with a sat. aq. NaHCO₃ solution (1 L×3) then with brine (1 L), dried (Na₂SO₄), filtered and concentrated under vacuum. The residue was triturated with MTBE (200 mL), the solid was filtered, and dried under vacuum to give the title compound as a white solid. ¹H NMR (400 MHz, CDCl₃) δ 4.83-4.73 (m, 1H), 4.01-3.91 (m, 2H), 3.90-3.80 (m, 2H), 2.87-2.82 (m, 2H), 2.66-2.57 (m, 2H), 2.27 (s, 3H), 1.44 (s, 9H). UPLC-MS-1a: Rt=1.31 min; MS m/z [M+H]⁺ 482.1/484.1.

### Intermediates C6a, C6b, C6c and C6d: O-(Tert-butyl) 1-methyl-6-(tosyloxy)-2-azaspiro[3.3]heptane-2-carbothioate

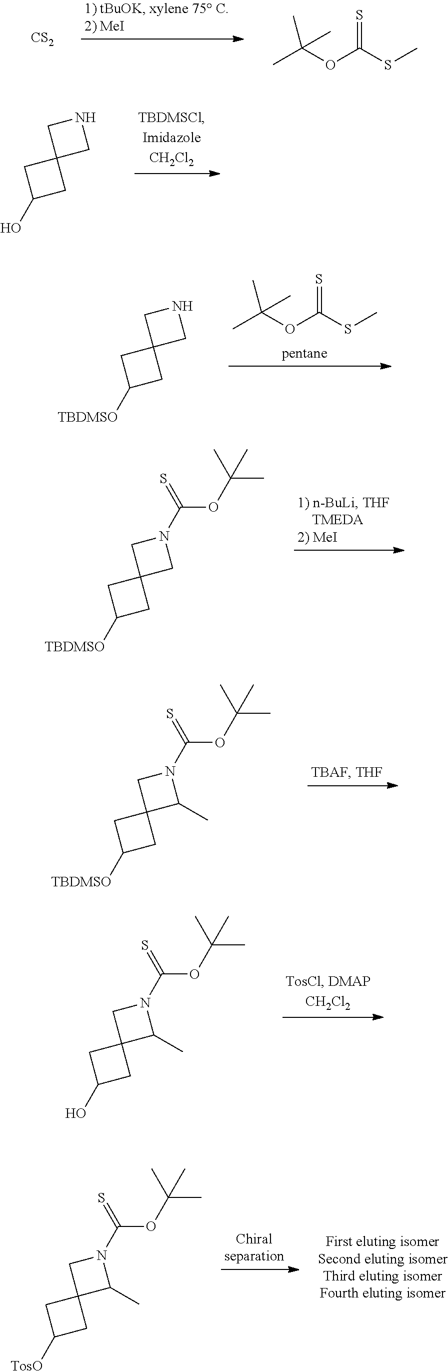

### Step 1: O-(Tert-butyl) S-methyl carbonodithioate

The title compound was prepared according to Mott, A. W.; barany, G. J. Chem. Soc. Perkin Trans. 1, 1984, 2615. Carbon disulfide (1.00 mL, 16.6 mmol) was added slowly to a solution of potassium t-butoxide (1.86 g, 16.6 mmol) in p-xylene (36 mL) at 75° C. The resulting yellow solid was collected by filtration, extensively washed with benzene and dried under vacuum to give a beige solid. This solid was suspended in Et₂O (22 mL) and iodomethane (1.55 mL, 24.9 mmol) was added slowly. The reaction mixture was stirred for 18 h. Then was filtered and the precipitate was washed with Et₂O. The filtrate was concentrated in a well ventilated hood (very unpleasant smelling) to give the title compound as a yellow oil. The compound was stored in the freezer to avoid decomposition. ¹H NMR (400 MHz, CDCl₃) δ 2.46 (s, 3H), 1.70 (s, 9H).

### Step 2: 6-((Tert-butyldimethylsilyl)oxy)-2-azaspiro[3.3]heptane-2-azaspiro[3.3]heptan-6-ol

To a solution of 2-azaspiro[3.3]heptan-6-ol hydrochloride (2.05 g, 13.7 mmol) and imidazole (1.96 g, 28.8 mmol) in CH₂Cl₂ (30 mL) cooled to 0-5° C. was added portionwise tert-butyldimethylsilyl chloride (2.48 g, 16.5 mmol) under a nitrogen atmosphere. The reaction mixture was stirred at RT for 2 d. Then the RM was poured into a sat. aq. NaHCO₃ solution and extracted with CH₂Cl₂ (×2). The combined organic extracts were washed with a sat. aq. NaHCO₃ solution (×2), dried (phase separator) and concentrated in vacuo to give the title compound. ¹H NMR (600 MHz, DMSO-d₆) δ 4.06 (m, 1H), 3.48-3.30 (m, 5H), 2.40 (m, 2H), 1.86 (m, 2H), 0.83 (s, 9H), 0.00 (s, 6H).

### Step 3: O-(tert-butyl) 6-((tert-butyldimethylsilyl)oxy)-2-azaspiro[3.3]heptane-2-carbothioate

To a solution of 6-((Tert-butyldimethylsilyl)oxy)-2-azaspiro[3.3]heptane-2-azaspiro[3.3] heptan-6-ol (Step 2, 1.60 g, 9.74 mmol) in pentane (6.5 mL) at 0-5° C. (ice-bath) was added O-(tert-butyl) S-methyl carbonodithioate (Step 1, 2.07 g, 8.19 mmol) and the reaction mixture was stirred at 0-5° C. for 1 h and then stirred at RT for 1 h. The reaction mixture was concentrated under reduced pressure and the crude residue was purified by normal phase chromatography (eluent: EtOAc in c-hexane 0 to 3%) to give the title compound as a beige solid. UPLC-MS-1a: Rt=1.63 min; MS m/z [M-tBu]⁺188.2.

### Step 4: O-(tert-butyl) 6-((tert-butyldimethylsilyl)oxy)-1-methyl-2-azaspiro[3.3]heptane-2-carbothioate

The title compound was prepared according to David M. Hodgson, D.; Mortimer, C. L.; McKenna, J. M. Org. Lett. 2015, 17, 330. To a solution of O-(tert-butyl) 6-((tert-butyldimethylsilyl)oxy)-2-azaspiro[3.3]heptane-2-carbothioate (Step 3, 1.90 g, 5.53 mmol) in dry THF (28 mL) was added under argon at −75° C. TMEDA (2.00 mL, 13.3 mmol) followed by the dropwise addition of s-BuLi (1.4M in c-hexane, 6.71 mL, 9.40 mmol). The reaction mixture was stirred for 30 min at −75° C., iodomethane (1.04 mL, 16.6 mmol) was slowly added and the reaction mixture was stirred for 20 min at −75° C., then warmed to RT and further stirred for 1 h. The reaction mixture was poured into a sat. aq. NH₄Cl solution, and extracted with EtOAc (×3). The combined organic extracts were washed with brine, dried (phase separator) and concentrated under reduced pressure to give the title compound which was used without further purification in the next step. UPLC-MS-1a: Rt=1.65 min; MS m/z [M-tBu]⁺302.3.

### Step 5: O-(tert-butyl) 6-hydroxy-1-methyl-2-azaspiro[3.3]heptane-2-carbothioate

To a solution of O-(tert-butyl) 6-((tert-butyldimethylsilyl)oxy)-1-methyl-2-azaspiro[3.3]heptane-2-carbothioate (Step 4, 2.00 g, 5.59 mmol) in THF (35 mL) was added TBAF (1M in THF, 11.2 mL, 11.2 mmol) and the reaction mixture was stirred under nitrogen for 2 min. The reaction mixture was poured into a sat. aq. NaHCO₃ solution and extracted with CH₂Cl₂ (×3). The combined organic extracts were dried (phase separator), concentrated in vacuo and the crude residue was purified by normal phase chromatography (eluent: EtOAc in c-hexane 20%) to give the title compound as a yellow oil. UPLC-MS-1a: Rt=0.96 min; MS m/z [M-tBu]⁺188.1.

### Step 6: O-(tert-butyl) 1-methyl-6-(tosyloxy)-2-azaspiro[3.3]heptane-2-carbothioate

To a solution of O-(tert-butyl) 6-hydroxy-1-methyl-2-azaspiro[3.3]heptane-2-carbothioate (Step 5, 1.12 g, 4.60 mmol) in CH₂Cl₂ (23 mL) under nitrogen atmosphere were added DMAP (0.73 g, 5.98 mmol) and Tosyl-Cl (1.05 g, 5.52 mmol) and the reaction mixture was stirred at RT for 14 h. The reaction mixture was poured into a sat. aq. NaHCO₃ solution and extracted with CH₂Cl₂ (×2). The combined organic extracts were dried (phase separator) and evaporated. The crude residue was purified by normal phase chromatography (eluent: EtOAc in c-hexane 0 to 15%) to give the title compound as a white foam. The isomers were separated by chiral C-HPLC-4 (mobile phase: n-heptane/EtOH 70/30+0.05% DEA) to give the title compound Intermediate C6a as the first eluting peak: (C-HPLC-5 (mobile phase: n-heptane/EtOH 70/30+0.05% DEA) Rt=5.00 min), UPLC-MS-1a: Rt=1.34 min; MS m/z [M+H]⁺; 342.2, the title compound Intermediate C6b as the second eluting peak: (C-HPLC-5 (mobile phase: n-heptane/EtOH 70/30+0.05% DEA): Rt=5.52 min), UPLC-MS-1a: Rt=1.33 min; MS m/z [M+H]⁺ 342.2, the title compound Intermediate C6c as the third eluting peak: (C-HPLC-5 (mobile phase: n-heptane/EtOH 70/30+0.05% DEA): Rt=6.40 min), UPLC-MS-1a: Rt=1.33 min; MS m/z [M+H]⁺ 342.2 and the title compound Intermediate C6d as the fourth eluting peak: (C-HPLC-5 (mobile phase: n-heptane/EtOH 70/30+0.05% DEA): Rt=11.65 min), UPLC-MS-1a: Rt=1.34 min; MS m/z [M+H]⁺ 342.2.

### Intermediate C7: Tert-butyl 1-methyl-6-(tosyloxy)-2-azaspiro[3.3]heptane-2-carboxylate

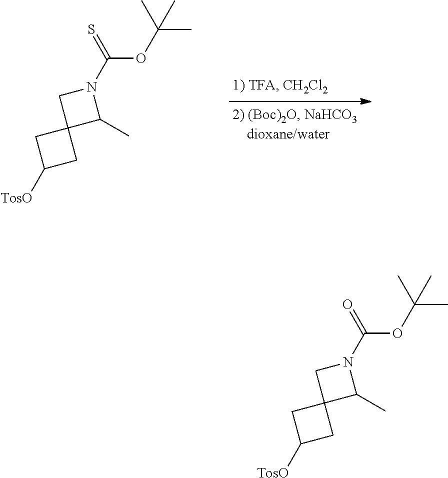

To a solution of o-(tert-butyl) 1-methyl-6-(tosyloxy)-2-azaspiro[3.3]heptane-2-carbothioate (Intermediate C6c, 1.65 g, 4.15 mmol) in CH₂Cl₂ (20 mL) was added TFA (0.64 mL, 8.30 mmol) and the reaction mixture was stirred at RT for 2 h. TFA (0.64 mL, 8.30 mmol) was added and the RM was further stirred at RT for 1.5 h. The RM was concentrated under reduced pressure. The crude residue was dissolved in dioxane/water (ratio 1/1, 28 mL) and cooled to 0-5° C., NaHCO₃ (2.09 g, 24.9 mmol) followed by (Boc)₂O (4.82 mL, 20.75 mmol) were added and the RM was allowed to reach RT and stirred at RT for 1 h. The RM was poured into water and extracted with CH₂Cl₂ (×3). The combined organic layers were dried (phase separator) and concentrated. The crude residue was purified by normal phase chromatography (eluent: EtOAc in heptane from 0 to 60%) to give the title compound as a colorless oil. UPLC-MS-4: Rt=1.18 min; MS m/z [M+H-Boc]⁺282.2.

### Intermediate C8: Tert-butyl 1-methyl-6-(tosyloxy)-2-azaspiro[3.3]heptane-2-carboxylate

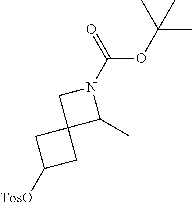

The title compound was prepared by a method similar to that of tert-butyl 1-methyl-6-(tosyloxy)-2-azaspiro[3.3]heptane-2-carboxylate (Intermediate C7) using o-(tert-butyl) 1-methyl-6-(tosyloxy)-2-azaspiro[3.3]heptane-2-carbothioate (Intermediate C6a) instead of Intermediate C6c. UPLC-MS-4: Rt=1.18 min; MS m/z [M+H-Boc]⁺282.2.

### Intermediate C9: Tert-butyl 6-(tosyloxy)-2-azaspiro[3.4]octane-2-carboxylate

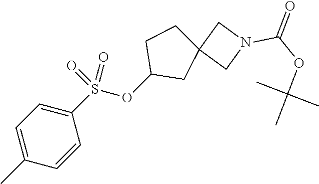 [SMILES: Cc1ccc(S(=O)(=O)OC2CCC3(C2)CN(C(=O)OC(C)(C)C)C3)cc1]

To tert-butyl 6-hydroxy-2-azaspiro[3.4]octane-2-carboxylate (1.00 g, 4.40 mmol) in CH₂Cl₂ (20 mL) under inert atmosphere were added DMAP (0.70 g, 5.72 mmol) and 4-toluenesulfonyl chloride (1.01 g, 5.28 mmol). The reaction mixture was stirred at RT for 16 h. The RM was poured into water and extracted twice with CH₂Cl₂. The combined organic phases were dried (Na₂SO₄), filtered and evaporated. The crude residue was purified by normal phase chromatography (eluent: EtOAc in c-hexane from 0 to 50%) to give the title compound as a colorless oil. UPLC-MS-1a: Rt=1.18 min; MS m/z [M+H]⁺ 382.4; [M+H-Boc]⁺282.3.

### Method-C10 for the Preparation of Intermediate C10: Tert-butyl 6-(3-(4-acetyl-2,2-dimethylpiperazin-1-yl)-4-bromo-5-methyl-1H-pyrazol-1-yl)-2-azaspiro[3.3]heptane-2-carboxylate

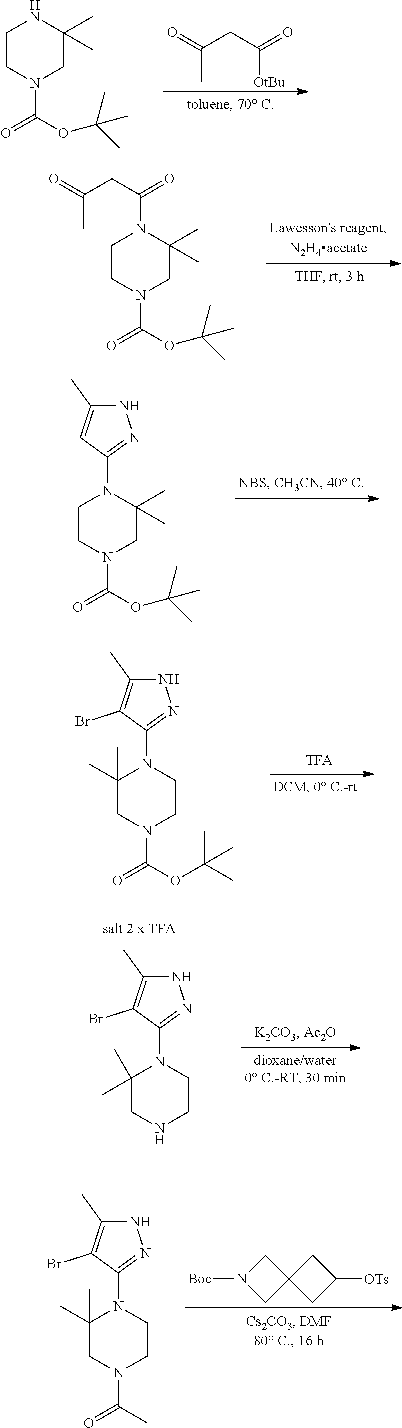

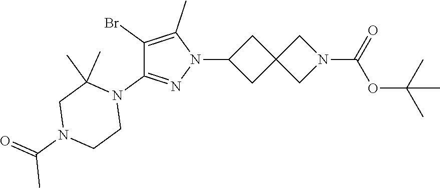 [SMILES: CC(=O)N1CCN(c2nn(C3CC4(C3)CN(C(=O)OC(C)(C)C)C4)c(C)c2Br)C(C)(C)C1]

### Step 1: Tert-butyl 3,3-dimethyl-4-(3-oxobutanoyl)piperazine-1-carboxylate

A solution of tert-butyl 3,3-dimethylpiperazine-1-carboxylate (CAS 259808-67-8, 2.70 kg, 10.7 mol) and tert-butyl 3-oxobutanoate (3.39 kg, 21.4 mol) in toluene (16.4 L) was stirred at 70° C. for 15 h. The reaction mixture was concentrated under vacuum to give a dark yellow oil. The crude product was purified by normal phase chromatography (eluent: EtOAc in n-heptane 0 to 50%) to give the title compound as a yellow oil. UPLC-MS-1a: Rt=0.85 min; MS m/z [M+H]⁺ 299.2.

### Step 2: Tert-butyl 3,3-dimethyl-4-(5-methyl-1H-pyrazol-3-yl)piperazine-1-carboxylate

A mixture of tert-butyl 3,3-dimethyl-4-(3-oxobutanoyl)piperazine-1-carboxylate (Step 1, 2.46 kg, 7.25 mol) and Lawesson's reagent (2,4-bis(4-methoxyphenyl)-1,3,2,4-dithiadiphosphetane-2,4-disulfide) (5.87 kg, 14.5 mol) in THF (61.5 L) was treated with hydrazine acetate (20.5 g, 14.5 mol) at 25-30° C. The reaction mixture was stirred at RT for 3 h. The reaction mixture was poured into ice-water (160 L). The separated aqueous layer was extracted with EtOAc (20.0 L×2). The combined organic layers were washed with saturated aqueous NaHCO₃ solution (98.0 L×2) then with brine (98.0 L×2), dried (Na₂SO₄), filtered and concentrated under vacuum. The crude product was purified by normal phase chromatography (eluent: EtOAc in n-heptane 0 to 50%) to give the title compound as a gray solid. UPLC-MS-1a: Rt=0.93 min; MS m/z [M+H]⁺ 295.2.

### Step 3: Intermediate C11: Tert-butyl 4-(4-bromo-5-methyl-1H-pyrazol-3-yl)-3,3-dimethylpiperazine-1-carboxylate

To an ice-cooled solution of tert-butyl 3,3-dimethyl-4-(5-methyl-1H-pyrazol-3-yl)piperazine-1-carboxylate (Step 2, 340 g, 1.13 mol) in acetonitrile (6.80 L) was added NBS (230 g, 1.27 mol) portion wise at 0° C. After 5 min, the reaction was complete. The reaction mixture was diluted with EtOAc (13.6 L) and washed with ice water (13.6 L), 0.4M aqueous NaS₂O₃ solution (22.0 L), saturated aqueous NaHCO₃ solution (22.0 L), then with brine (22.0 L×2). The combined aqueous layers were re-extracted with EtOAc (10.0 L). The combined organic layers were dried (Na₂SO₄), filtered, and concentrated under vacuum to give the title compound as a white solid which was used without purification in the next step. UPLC-MS-1a: Rt=1.18 min; MS m/z [M+H]⁺ 373.1/375.1.

### Step 4: 1-(4-Bromo-5-methyl-1H-pyrazol-3-yl)-2,2-dimethylpiperazine

To a solution of tert-butyl 4-(4-bromo-5-methyl-1H-pyrazol-3-yl)-3,3-dimethylpiperazine-1-carboxylate (Step 3, 324 g, 832 mmol) in CH₂Cl₂ (7.77 L) was added TFA (950 g, 8.30 mol) at 0° C. After completion of the reaction, the reaction mixture was concentrated under vacuum to give the title compound as a trifluoroacetate salt which was used without purification in the next step. UPLC-MS-1a: Rt=0.50 min; MS m/z [M+H]⁺ 273.0/275.0.

### Step 5: 1-(4-(4-Bromo-5-methyl-1H-pyrazol-3-yl)-3,3-dimethylpiperazin-1-yl)ethan-1-one

To a solution of 1-(4-bromo-5-methyl-1H-pyrazol-3-yl)-2,2-dimethylpiperazine as a trifluoroacetate salt (Step 4, 550 g, 832 mmol) in 1,4-dioxane (4.67 L)/water (4.67 L) was added K₂CO₃ (345 g, 2.50 mol) at 0° C. The reaction mixture was stirred at 0° C. for 30 min then acetic anhydride (128 g, 1.25 mol) was added. The resulting mixture was warmed up to 25° C. After completion of the reaction, the reaction mixture was quenched with cold water (1.00 L) and extracted with EtOAc (3.00 L×3). The combined organic layers were washed with saturated aqueous NaHCO₃ solution (500 mL) then with brine (500 mL), dried (Na₂SO₄), filtered and concentrated under vacuum to give the title compound as a white solid. UPLC-MS-1a: Rt=0.78 min; MS m/z [M+H]⁺ 315.1/317.1.

### Step 6: Tert-butyl 6-(3-(4-acetyl-2,2-dimethylpiperazin-1-yl)-4-bromo-5-methyl-1H-pyrazol-1-yl)-2-azaspiro[3.3]heptane-2-carboxylate

To a solution of 1-(4-(4-bromo-5-methyl-1H-pyrazol-3-yl)-3,3-dimethylpiperazin-1-yl)ethan-1-one (Step 5, 230 g, 670 mmol) in dry DMF (3.20 L) was added tert-butyl 6-(tosyloxy)-2-azaspiro[3.3]heptane-2-carboxylate (Intermediate C2, 246 g, 670 mmol) and Cs₂CO₃ (540 g, 1.67 mol) at 25° C. then the reaction mixture was stirred at 80° C. under inert atmosphere for 16 h. The reaction mixture was cooled to 25° C. and diluted with water (12.8 L), then extracted with MTBE (2.00 L×4). The combined organic layers were washed with brine, dried (Na₂SO₄), filtered, and concentrated under vacuum to give a yellow oil. The crude product was purified by normal phase chromatography (eluent: EtOAc in n-heptane 0 to 50%) to give the title compound as a white solid. ¹H NMR (600 MHz, DMSO-d₆) δ 4.75-4.64 (m, 1H), 3.99-3.75 (m, 4H), 3.58-3.47 (m, 2H), 3.32-3.27 (m, 2H), 3.02-2.88 (m, 2H), 2.65-2.57 (m, 2H), 2.40-2.60 (m, 2H), 2.16 (s, 3H), 2.05 (s, 1.5H), 2.00 (s, 1.5H), 1.36 (s, 9H), 1.16 (s, 3H), 1.13 (s, 3H); UPLC-MS-1a: Rt=1.23 min; MS m/z [M+H]⁺ 510.2/512.2 .

Method-C10a: similar to Method-C10 except that in Step 3 AIBN (0.1 eq) was added to the reaction.

Method-C10b: similar to Method-C10 except that Step 6 was performed in DMA instead of DMF.

The following intermediates C12 and C13 were prepared using analogous methods to Method-C10 from intermediates commercially available (in Step 1).

| Intermediate | Structure | Precursor | Characterization data |
|---|---|---|---|
| C12 | 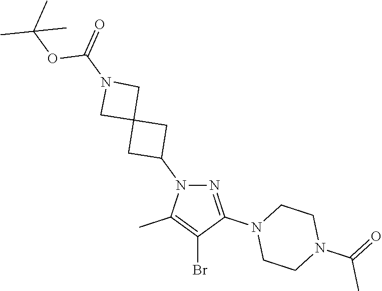 [SMILES: CC(=O)N1CCN(c2nn(C3CC4(C3)CN(C(=O)OC(C)(C)C)C4)c(C)c2Br)CC1] | Using Method- C10a from tert- butyl piperazine-1- carboxylate (Step 1) | UPLC-MS-2a: Rt = 1.01 min; MS m/z [M + H]⁺ 482.1/484.1. |
| | Tert-butyl 6-(3-(4- | | |
| | acetylpiperazin-1-yl)-4- | | |
| | bromo-5-methyl-1H-pyrazol- | | |
| | 1-yl)-2-azaspiro[3.3]heptane- | | |
| | 2-carboxylate | | |
| C13 | 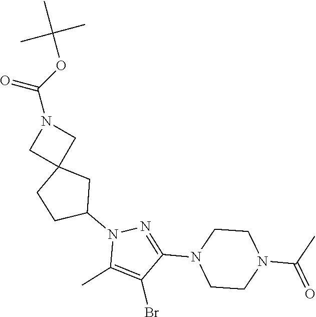 [SMILES: CC(=O)N1CCN(c2nn(C3CCC4(C3)CN(C(=O)OC(C)(C)C)C4)c(C)c2Br)CC1] | Using Method- C10b starting from the tert- butyl piperazine-1- carboxylate and using Intermediate C9 | UPLC-MS-1a: Rt = 1.19 min; MS m/z [M + H]⁺ 496.4/498.4. |
| | Tert-butyl 6-(3-(4- | | |
| | acetylpiperazin-1-yl)-4- | | |
| | bromo-5-methyl-1H-pyrazol- | | |
| | 1-yl)-2-azaspiro[3.4]octane- | | |
| | 2-carboxylate | | |

### Intermediate C14: 1-(4-(4-Bromo-3-methyl-1H-pyrazol-5-yl)piperazin-1-yl)ethan-1-one

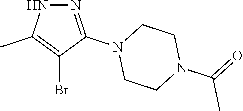 [SMILES: CC(=O)N1CCN(c2n[nH]c(C)c2Br)CC1]

The title compound was prepared using analogous methods to method-C10a Step 1-6 starting form tert-butyl piperazine-1-carboxylate. UPLC-MS-1a: Rt=0.64 min; MS m/z [M+H]⁺ 287.1/289.1.

### Intermediate C15: 1-(4-(4-(5-chloro-1-(tetrahydro-2H-pyran-2-yl)-1H-indazol-4-yl)-5-methyl-1H-pyrazol-3-yl)piperazin-1-yl)ethan-1-one

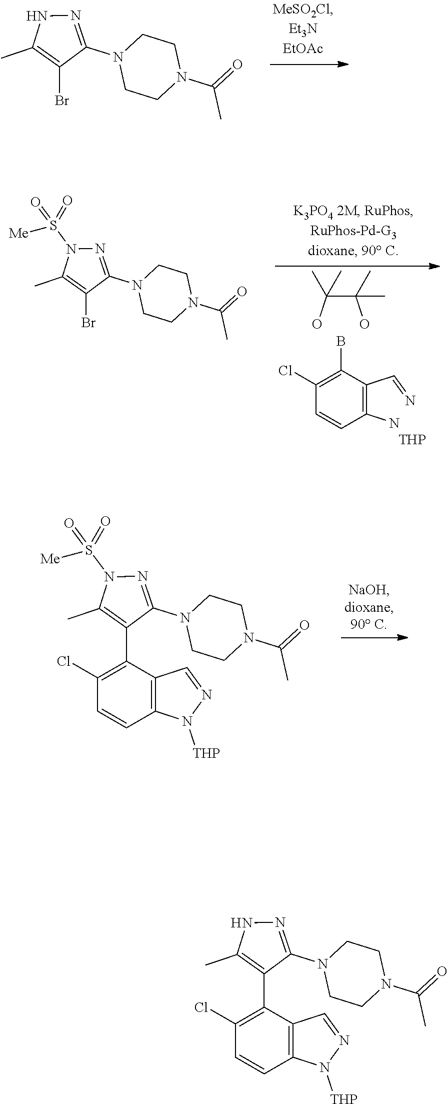

### Step 1: 1-(4-(4-Bromo-5-methyl-1-(methylsulfonyl)-1H-pyrazol-3-yl)piperazin-1-yl)ethan-1-one

To a stirred solution of 1-(4-(4-bromo-3-methyl-1H-pyrazol-5-yl)piperazin-1-yl)ethan-1-one (Intermediate C14, 2.40 g, 8.36 mmol) in ethylacetate (10 mL) were successively added Et₃N (1.86 mL, 13.4 mmol) and methanesulfonyl chloride (0.98 mL, 12.5 mmol). The reaction mixture was stirred at Rt for 2 h. The RM was diluted with water (50 mL), extracted with EtOAc (×2) and the combined organic extracts were washed with water (×2). The water layers were extracted again with nBuOH and the combined organic extracts were dried and concentrated. The crude reside was purified by normal phase chromatography (eluent: (CH₂Cl₂+10% MeOH) in CH₂Cl₂ from 0 to 20%) to give the title compound. UPLC-MS-1a: Rt=0.78 min; MS m/z [M+H]⁺ 365.1/367.1.

### Step 2: 1-(4-(4-(5-Chloro-1-(tetrahydro-2H-pyran-2-yl)-1H-indazol-4-yl)-5-methyl-1-(methylsulfonyl)-1H-pyrazol-3-yl)piperazin-1-yl)ethan-1-one

To a mixture of 1-(4-(4-bromo-5-methyl-1-(methylsulfonyl)-1H-pyrazol-3-yl)piperazin-1-yl)ethan-1-one (1.64 g, 4.49 mmol), 5-chloro-1-(tetrahydro-2H-pyran-2-yl)-4-(4,4,5,5-tetramethyl-1,3,2-dioxaborolan-2-yl)-1H-indazole (Intermediate D7, 1.95 g, 5.39 mmol), potassiumcarbonate (6.74 ml, 13.47 mmol) in dioxane (15 mL) was added RuPhos (0.21 g, 0.45 mmol) and Ruphos-Pd-G3 (0.39 g, 0.45 mmol). The reaction mixture was stirred under an argon atmosphere at 90° C. for 30 min. Water was added and the RM was extracted with EtOAc (×2). The combined organic extracts were washed with water, dried and concentrated. The crude residue was purified by normal phase chromatography (eluent: (CH₂Cl₂+10% MeOH) in CH₂Cl₂ from 0 to 20%) to give the title compound. UPLC-MS-1a: Rt=1.01, 1.02 min; MS m/z [M+H]⁺ 521.5/523.5.

### Step 3: 1-(4-(4-(5-Chloro-1-(tetrahydro-2H-pyran-2-yl)-1H-indazol-4-yl)-5-methyl-1H-pyrazol-3-yl)piperazin-1-yl)ethan-1-onem

A solution of 1-(4-(4-(5-chloro-1-(tetrahydro-2H-pyran-2-yl)-1H-indazol-4-yl)-5-methyl-1-(methylsulfonyl)-1H-pyrazol-3-yl)piperazin-1-yl)ethan-1-one (Step 2, 1.57 g, 3.01 mmol) in dioxane (15 mL) and sodium hydroxide (2M, 4.52 mL, 9.04 mmol) was stirred at 90° C. for 45 min. The RM was diluted with EtOAc (10 mL) and washed a 5% bicarbonate solution and with water (×2). The combined water layers were extracted with nBuOH and the combined organic layers were dried (Na₂SO₄), filtered and evaporated to give the title compound which was used without further purification in the nest step. UPLC-MS-1a: Rt=0.89, 0.90 min; MS m/z [M+H]⁺ 443.5/445.5.

### Method-C16 for the preparation of Intermediate C16: 4-(3-((S)-4-(((R)-1,4-Dioxan-2-yl)methyl)-2-ethyl-2-methylpiperazin-1-yl)-5-methyl-1H-pyrazol-4-yl)-5-chloro-6-methyl-1-(tetrahydro-2H-pyran-2-yl)-1H-indazole

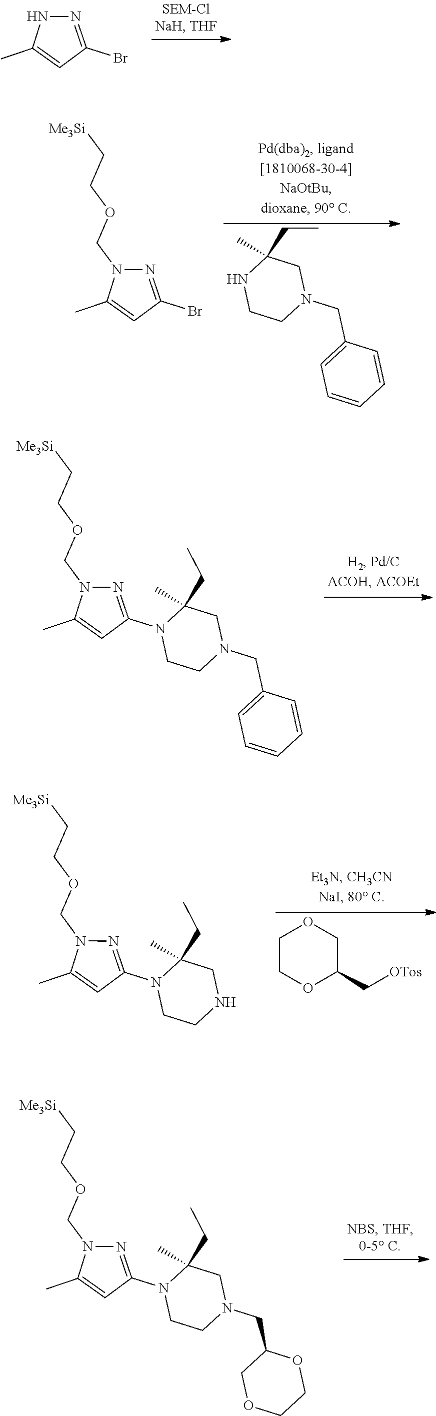

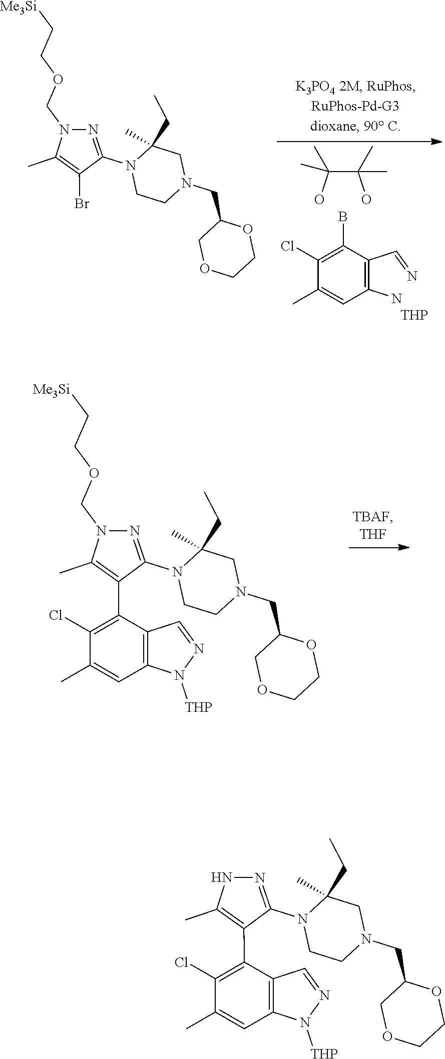

### Step 1: 3-Bromo-5-methyl-1-((2-(trimethylsilyl)ethoxy)methyl)-1H-pyrazole

To a solution of 3-bromo-5-methyl-1H-pyrazole (6.00 g, 37.3 mmol) in THF (180 ml) under nitrogen atmosphere was added NaH (60% in grease, 3.73 g, 93.0 mmol) at 0° C. and the mixture was stirred for 1 h. SEMCI (9.25 mL, 52.2 mmol) was added dropwise and the RM was stirred at 0° C. for 30 min, then allowed to reach RT and was further stirred for 16 h. The reaction mixture was quenched with water and extracted with EtOAc (3×). The combined organic layers were dried (phase separator) and concentrated under reduced pressure. The crude residue was purified by normal phase chromatography (eluent: EtOAc in heptane from 0 to 10%) to give the title compound as a mixture of 2 regioisomers (yellow oil). UPLC-MS-4: Rt=1.36 and 1.40 min; MS m/z [M+H]⁺ 291.1/293.1.

### Step 2: (S)-4-Benzyl-2-ethyl-2-methyl-1-(5-methyl-1-((2-(trimethylsilyl)ethoxy)methyl)-1H-pyrazol-3-yl)piperazine

In an ace tube were added 3-bromo-5-methyl-1-((2-(trimethylsilyl)ethoxy)methyl)-1H-pyrazole (Step 1 mixture of 2 regioisomers, 9.38 g, 32.2 mmol), (S)-1-benzyl-3-ethyl-3-methylpiperazine (Intermediate A32, 4.69 g, 21.5 mmol), Pd(dba)₂ (0.93 g, 1.61 mmol), bis(3,5-bis(trifluoromethyl)phenyl)(2′,6′-bis(dimethylamino)-3,6-dimethoxybiphenyl-2-yl)phosphine (CAS [1810068-30-41], 1.22 g, 1.61 mmol). The tube was placed under nitrogen atmosphere and dioxane (100 mL) followed by NaOtBu (2M in THF, 16.1 mL, 32.2 mmol) were added. The reaction mixture was heated at 85° C. for 16 h. To complete the reaction, Pd(dba)₂ (0.93 g, 1.61 mmol), bis(3,5-bis(trifluoromethyl)phenyl)(2′,6′-bis(dimethylamino)-3,6-dimethoxybiphenyl-2-yl)phosphine (1.22 g, 1.61 mmol) and NaOtBu (2M in THF, 16.1 mL, 32.2 mmol) were added again and the RM was further stirred at RT for 16 h. The RM was poured into an aq. sat. NaHCO₃ solution and extracted with CH₂Cl₂ (×3). The combined organic layers were dried (phase separator) and concentrated under reduced pressure. The crude residue was purified twice by normal phase chromatography (1-using a RediSep cartridge and eluting with: EtOAc in heptane from 0 to 50%, 2-using a RediSep GOLD cartridge and eluting with: EtOAc in heptane from 0 to 20) to give the title compound as a single regioisomer (orange oil). ¹H NMR (400 MHz, DMSO-d₆) δ 7.32-7.34 (m, 5H), 5.66 (s, 1H), 5.19 (s, 2H), 3.50-3.38 (m, 4H), 3.15-2.99 (m, 2H), 2.42 (m, 2H), 2.31 (m, 1H), 2.19 (s, 3H), 2.07-1.95 (m, 2H), 1.42-1.33 (m, 1H), 1.13 (s, 3H), 0.79 (t, 2H), 0.66 (t, 3H), 0.07 (s, 9H); UPLC-MS-4: Rt=1.16 min; MS m/z [M+H]⁺ 229.1.

### Step 3: (S)-2-Ethyl-2-methyl-1-(5-methyl-1-((2-(trimethylsilyl)ethoxy)methyl)-1H-pyrazol-3-yl)piperazine

A solution of (S)-4-benzyl-2-ethyl-2-methyl-1-(5-methyl-1-((2-(trimethylsilyl)ethoxy)methyl)-1H-pyrazol-3-yl)piperazine (Step 2, 3.59 g, 8.38 mmol) in EtOAc (100 mL) and AcOH (4.80 mL, 84 mmol) was evacuated and back-filled with nitrogen (×3) before the addition of Pd/C (10%, 1.47 g, 1.38 mmol). The mixture was evacuated and back-filled again with nitrogen (×2) then evacuated and back-filled with hydrogen (×3) and the RM was stirred at RT under an atmosphere of hydrogen (ballon) for 24 h. The RM was filtered over a pad of celite, washed with EtOAc and the filtrate was concentrated. The crude residue was dissolved in CH₂Cl₂, a sat. aq. NaHCO₃ solution was added and the 2 layers were separated. The aqueous layer was back-extracted with CH₂Cl₂ and the combined organic extracts were dried (phase separator) and concentrated. The crude residue was purified by normal phase chromatography (eluent: CH₂Cl₂/MeOH 8/2 in CH₂Cl₂ from 0 to 100%) to give the title compound. UPLC-MS-4: Rt=0.81 min; MS m/z [M+H]⁺ 339.1.

### Step 4: (S)-4-(((R)-1,4-Dioxan-2-yl)methyl)-2-ethyl-2-methyl-1-(5-methyl-1-((2-(trimethylsilyl)ethoxy)methyl)-1H-pyrazol-3-yl)piperazine

To a solution of (S)-2-ethyl-2-methyl-1-(5-methyl-1-((2-(trimethylsilyl)ethoxy)methyl)-1H-pyrazol-3-yl)piperazine (Step 3, 3.68 g, 10.9 mmol) in CH₃CN (90 mL) was added under argon (S)-(1,4-dioxan-2-yl)methyl 4-methylbenzenesulfonate (Intermediate B1, 4.44 g, 16.3 mmol), triethylamine (4.54 mL, 32.6 mmol) and NaI (1.63 g, 10.9 mmol) and the reaction mixture was stirred at 80° C. for 48 h. The reaction mixture was diluted with a sat. aq. NaHCO₃ solution, extracted with EtOAc (×3) and the combined organic extracts were washed with brine, dried (Na₂SO₄), filtered and evaporated. The crude residue was purified by normal phase chromatography (eluent: EtOAc in n-heptane 0 to 100%). UPLC-MS-4: Rt=0.87 min; MS m/z [M+H]⁺ 439.6.

### Step 5: (S)-4-(((R)-1,4-Dioxan-2-yl)methyl)-1-(4-bromo-5-methyl-1-((2-(trimethylsilyl)ethoxy)methyl)-1H-pyrazol-3-yl)-2-ethyl-2-methylpiperazine

To an ice-cooled solution of (S)-4-(((R)-1,4-dioxan-2-yl)methyl)-2-ethyl-2-methyl-1-(5-methyl-1-((2-(trimethylsilyl)ethoxy)methyl)-1H-pyrazol-3-yl)piperazine (Step 4, 3.65 g, 8.33 mmol) in THF (80 mL) was added NBS (1.63 g, 9.16 mmol) and the mixture was stirred under N₂ atmosphere at 0° C. After completion (30 min), the reaction mixture was concentrated and the crude residue was purified by normal phase chromatography (eluent: EtOAc in n-heptane 0 to 50%). UPLC-MS-4: Rt=1.05 min; MS m/z [M+H]⁺ 517.4/519.4.

### Step 6: 4-(3-((S)-4-(((R)-1,4-Dioxan-2-yl)methyl)-2-ethyl-2-methylpiperazin-1-yl)-5-methyl-1-((2-(trimethylsilyl)ethoxy)methyl)-1H-pyrazol-4-yl)-5-chloro-6-methyl-1-(tetrahydro-2H-pyran-2-yl)-1H-indazole

To a mixture of (S)-4-(((R)-1,4-dioxan-2-yl)methyl)-1-(4-bromo-5-methyl-1-((2-(trimethylsilyl)ethoxy)methyl)-1H-pyrazol-3-yl)-2-ethyl-2-methylpiperazine (Step 5, 6.72 g, 3.48 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 3.80 g, 10.1 mmol), RuPhos (0.31 g, 0.72 mmol) and RuPhos-Pd-G3 (0.56 g, 0.67 mmol) in dioxane (65 mL) was added K₃PO₄ (2 M in water, 10.1 mL, 20.2 mmol) and the reaction mixture was stirred at 85° C. for 2 h under nitrogen atmosphere. The reaction mixture was poured into a sat. aq. NaHCO₃ solution and extracted with EtOAc (×3). The combined organic extracts were dried (Na₂SO₄), filtered and concentrated. The crude residue was diluted with THF (25 mL), SiliaMetS®Thiol (2.67 mmol) was added and the mixture swirled for 1 h at 40° C. The mixture was filtered, the filtrate was concentrated and the crude residue was purified by normal phase chromatography (eluent: EtOAc in n-heptane 0 to 100%) to give the title compound as a yellow foam. UPLC-MS-4: Rt=1.21 min; MS m/z [M+H]⁺ 687.8/689.8.

### Step 7: 4-(3-((S)-4-(((R)-1,4-Dioxan-2-yl)methyl)-2-ethyl-2-methylpiperazin-1-yl)-5-methyl-1H-pyrazol-4-yl)-5-chloro-6-methyl-1-(tetrahydro-2H-pyran-2-yl)-1H-indazole

To a solution of 4-(3-((S)-4-(((R)-1,4-dioxan-2-yl)methyl)-2-ethyl-2-methylpiperazin-1-yl)-5-methyl-1-((2-(trimethylsilyl)ethoxy)methyl)-1H-pyrazol-4-yl)-5-chloro-6-methyl-1-(tetrahydro-2H-pyran-2-yl)-1H-indazole (Step 6, 4.52 g, 5.79 mmol) in THF (58 mL) was added TBAF (1 M in THF, 57.9 mL, 57.9 mmol) and the reaction mixture was stirred at 60° C. for 72 h. The reaction mixture was poured into a sat. aq. NH₄Cl solution and extracted with EtOAc (×3). The combined organic extracts were washed with a sat. aq. NaHCO₃ solution (×2), dried (phase separator) and concentrated. The crude residue was purified by normal phase chromatography (eluent: (CH₂Cl₂/MeOH 8/2) in CH₂Cl₂ from 0 to 10%) to give the title compound. UPLC-MS-4: Rt=0.82 and 0.83 min; MS m/z [M+H]⁺ 557.5/559.5.

### Intermediate C17: 5,6-Dichloro-4-(3-((R)-2,2-dimethyl-4-((4-(oxetan-3-yl)piperazin-1-yl)methyl)piperidin-1-yl)-5-methyl-1H-pyrazol-4-yl)-1-(tetrahydro-2H-pyran-2-yl)-1H-indazole

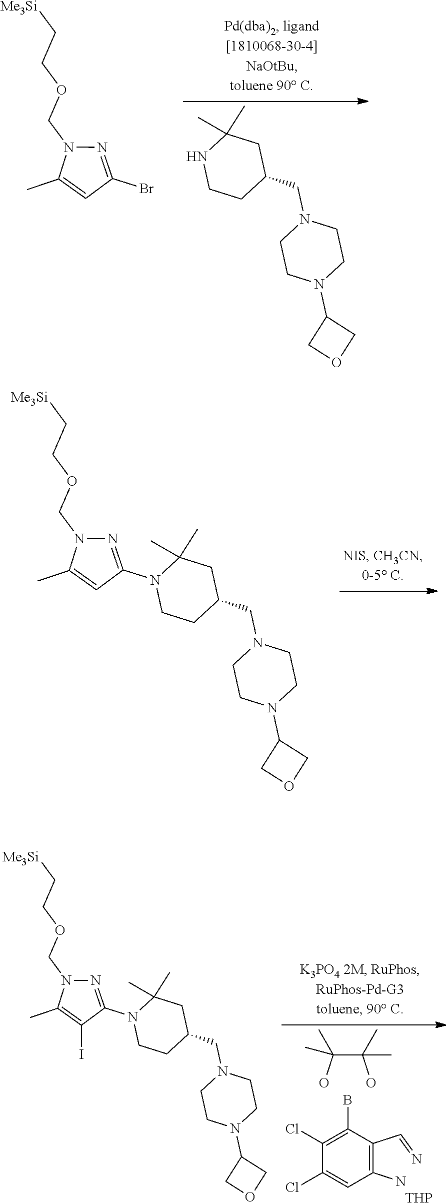

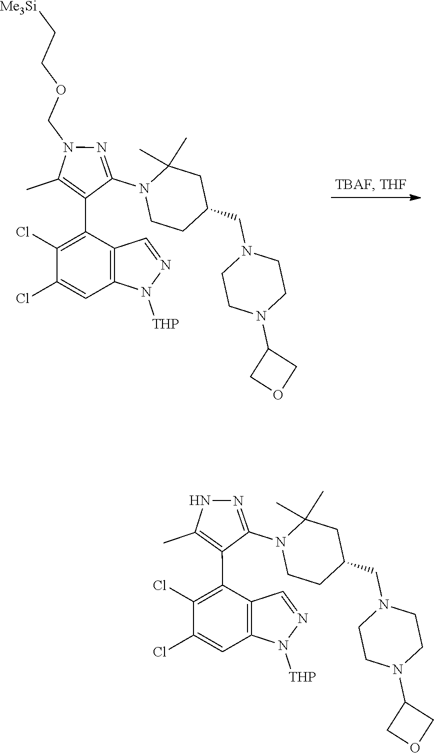

### Step 1: (R)-1-((2,2-Dimethyl-1-(5-methyl-1-((2-(trimethylsilyl)ethoxy)methyl)-1H-pyrazol-3-yl)piperidin-4-yl)methyl)-4-(oxetan-3-yl)piperazine

The title compound was prepared using analogous method to method-C16 (Step 2) using (R)-1-((2,2-dimethylpiperidin-4-yl)methyl)-4-(oxetan-3-yl)piperazine (Intermediate A78) and toluene as the solvent. UPLC-MS-4: Rt=0.78 min; MS m/z [M+H]⁺ 478.6.

### Step 2: (R)-1-((1-(4-lodo-5-methyl-1-((2-(trimethylsilyl)ethoxy)methyl)-1H-pyrazol-3-yl)-2,2-dimethylpiperidin-4-yl)methyl)-4-(oxetan-3-yl)piperazine

The title compound was prepared using analogous method to method-C16 (Step 5) starting from (R)-1-((2,2-dimethyl-1-(5-methyl-1-((2-(trimethylsilyl)ethoxy)methyl)-1H-pyrazol-3-yl)piperidin-4-yl)methyl)-4-(oxetan-3-yl)piperazine (Step 1) and using NIS in CH₃CN instead of NBS in THF. UPLC-MS-4: Rt=1.05 min; MS m/z [M+H]⁺ 604.3.

### Step 3: 5,6-Dichloro-4-(3-((R)-2,2-dimethyl-4-((4-(oxetan-3-yl)piperazin-1-yl)methyl)piperidin-1-yl)-5-methyl-1-((2-(trimethylsilyl)ethoxy)methyl)-1H-pyrazol-4-yl)-1-(tetrahydro-2H-pyran-2-yl)-1H-indazole

The title compound was prepared using analogous method to method-C16 (Step 6) starting from (R)-1-((1-(4-iodo-5-methyl-1-((2-(trimethylsilyl)ethoxy)methyl)-1H-pyrazol-3-yl)-2,2-dimethylpiperidin-4-yl)methyl)-4-(oxetan-3-yl)piperazine (Step 2) and 5,6-dichloro-1-(tetrahydro-2H-pyran-2-yl)-4-(4,4,5,5-tetramethyl-1,3,2-dioxaborolan-2-yl)-1H-indazole (intermediate D6) using toluene as the solvent. UPLC-MS-4: Rt=1.22 min; MS m/z [M+H]⁺ 746.6/748.6.

### Step 4: 5,6-Dichloro-4-(3-((R)-2,2-dimethyl-4-((4-(oxetan-3-yl)piperazin-1-yl)methyl)piperidin-1-yl)-5-methyl-1H-pyrazol-4-yl)-1-(tetrahydro-2H-pyran-2-yl)-1H-indazole

The title compound was prepared using analogous method to method-C16 (Step 7) starting from 5,6-dichloro-4-(3-((R)-2,2-dimethyl-4-((4-(oxetan-3-yl)piperazin-1-yl)methyl)piperidin-1-yl)-5-methyl-1-((2-(trimethylsilyl)ethoxy)methyl)-1H-pyrazol-4-yl)-1-(tetrahydro-2H-pyran-2-yl)-1H-indazole (Step 3). UPLC-MS-4: Rt=0.81, 0.82 and 0.86 min; MS m/z [M+H]⁺ 616.4/618.4.

### Intermediate C18: Tert-butyl 6-(3-(4-acetyl-2,2-dimethylpiperazin-1-yl)-5-cyclopropyl-1H-pyrazol-1-yl)-2-azaspiro[3.3]heptane-2-carboxylate

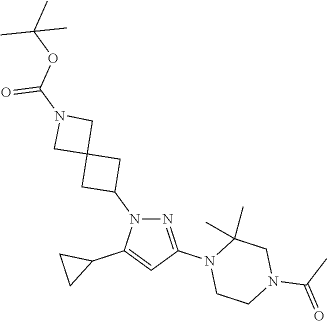 [SMILES: CC(=O)N1CCN(c2cc(C3CC3)n(C3CC4(C3)CN(C(=O)OC(C)(C)C)C4)n2)C(C)(C)C1]

### Step 1: Tert-butyl 6-(3-bromo-5-cyclopropyl-1H-pyrazol-1-yl)-2-azaspiro[3.3]heptane-2-carboxylate

To tert-butyl 6-(3,5-dibromo-1H-pyrazol-1-yl)-2-azaspiro[3.3]heptane-2-carboxylate (Intermediate C3, 100 mg, 0.24 mmol) and potassium cyclopropyltrifluoroborate (42.2 mg, 0.28 mmol) in dioxane (1.0 mL) were added under inert atmopshere Pd(Ph₃P)₄ (27.4 mg, 0.024 mmol) and Na₂CO₃ (2 M in H₂O, 249 μl, 0.50 mmol). The reaction mixture was degassed with N₂ and stirred at 95° C. for 16 h. More potassium cyclopropyltrifluoroborate (42.2 mg, 0.285 mmol), Pd(Ph₃P)₄ (27.4 mg, 0.024 mmol) and Na₂CO₃ (2 M in H₂O, 249 μl, 0.499 mmol) were added. The reaction mixture was stirred at 95° C. for 4 h. After cooling at RT, the reaction mixture was treated with sat. aq. NaHCO₃ solution, extracted twice with EtOAc and the combined organic phases were dried (Na₂SO₄), filtered and concentrated in vacuo. The crude residue was purified by normal phase chromatography (eluent: EtOAc in c-hexane from 0 to 40%) to give the title compound as a white gum. UPLC-MS-2a: Rt=1.25 min; MS m/z [M+H]⁺ 382.1/384.1.

### Step 2: Tert-butyl 6-(3-(4-acetyl-2,2-dimethylpiperazin-1-yl)-5-cyclopropyl-1H-pyrazol-1-yl)-2-azaspiro[3.3]heptane-2-carboxylate

To a mixture of tert-butyl 6-(3-bromo-5-cyclopropyl-1H-pyrazol-1-yl)-2-azaspiro[3.3]heptane-2-carboxylate (Step 1, 307 mg, 0.80 mmol), 1-(3,3-dimethylpiperazin-1-yl)ethan-1-one (Intermediate A13, 163 mg, 1.04 mmol), Pd(dba)₂ (46.2 mg, 0.08 mmol) and bis(3,5-bis(trifluoromethyl)phenyl)(2′,6′-bis(dimethylamino)-3,6-dimethoxybiphenyl-2-yl)phosphine (66.8 mg, 0.09 mmol) suspended in 1,4-dioxane (4 mL) was added NaOtBu (2 M in THF, 562 μl, 1.12 mmol). The reaction mixture was stirred at 85° C. for 16 h. The RM was treated with a sat. aq. NaHCO₃ solution, extracted with CH₂Cl₂ and the organic layers were dried (Na₂SO₄), filtered and concentrated in vacuo. The crude residue was purified by normal phase chromatography (eluent: MeOH in CH₂Cl₂ from 0 to 5%) to give the title compound as a yellow gum. UPLC-MS-2a: Rt=1.11 min; MS m/z [M+H]⁺ 458.3.

### Method-C19 for the preparation of Intermediate C19: Tert-butyl 6-(3-(4-acetylpiperazin-1-yl)-4-iodo-5-methyl-1H-pyrazol-1-yl)-2-azaspiro[3.3]heptane-2-carboxylate

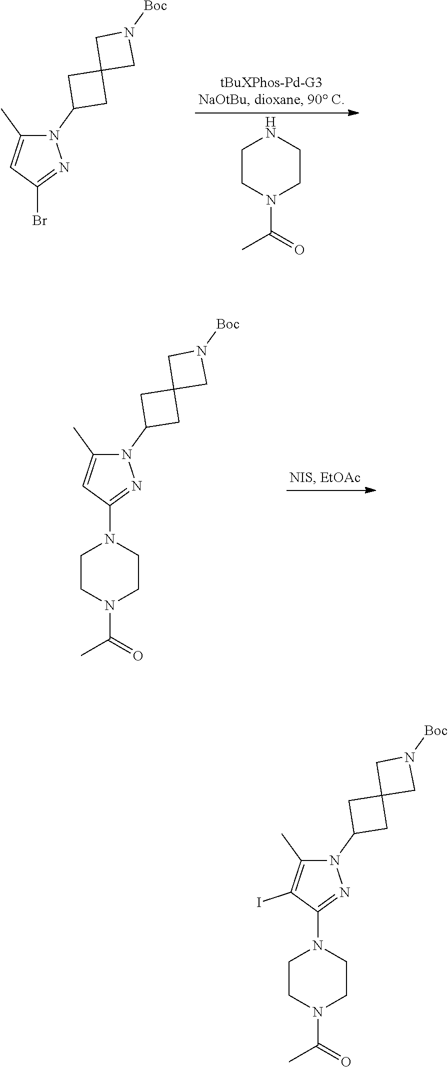

### Step 1: Tert-butyl 6-(3-(4-acetylpiperazin-1-yl)-5-methyl-1H-pyrazol-1-yl)-2-azaspiro[3.3]heptane-2-carboxylate

6-(3-Bromo-5-methyl-1H-pyrazol-1-yl)-2-azaspiro[3.3]heptane-2-carboxylate (Intermediate C1, 720 mg, 1.92 mmol) and 1-(piperazin-1-yl)ethan-1-one (497 mg, 3.84 mmol) were dissolved in 1,4-dioxane (12.0 mL) under Ar and degassed. NaOtBu (2M in THF, 2.88 mL, 5.76 mmol) followed by tBuXPhos-Pd-G3 (157 mg, 0.19 mmol) were added and the reaction mixture was stirred at 90° C. for 17 h. The reaction mixture was diluted with a sat. aq. NaHCO₃ solution and extracted with EtOAc. The combined organic layers were washed with brine, dried (Na₂SO₄), filtered and concentrated in vacuo. The crude residue was purified by normal phase chromatography (eluent: EtOAc in n-heptane 50 to 100%) to give the title compound as a foam. UPLC-MS-1a: Rt=0.98 min; MS m/z [M+H]⁺ 404.3.

### Step 2: Tert-butyl 6-(3-(4-acetylpiperazin-1-yl)-4-iodo-5-methyl-1H-pyrazol-1-yl)-2-azaspiro[3.3]heptane-2-carboxylate

Under Ar, to a solution of tert-butyl 6-(3-(4-acetylpiperazin-1-yl)-5-methyl-1H-pyrazol-1-yl)-2-azaspiro[3.3]heptane-2-carboxylate (Step 1, 380 mg, 0.93 mmol) in EtOAc (10.0 mL) was added NIS (281 mg, 1.21 mmol) and the reaction mixture was stirred at RT for 30 mi. The reaction mixture was diluted with a 10% aq. sodium thiosulfate solution and extracted with EtOAc. The combined organic layers were washed with brine, dried (Na₂SO₄), filtered and concentrated in vacuo to give the title compound as an off white solid. UPLC-MS-1a: Rt=1.16 min; MS m/z [M+H]⁺ 530.2.

Method-C19a: similar to Method-C19 except that in Step 3 NBS was used instead of NIS to give the corresponding 4.bromo pyrrazole.

The following intermediates C20 to C22 were prepared using analogous methods to method-19 from intermediates described in the intermediates synthesis section or commercially available (in Step 1).

| | | | Characterizing |
|---|---|---|---|
| Intermediate | Structure | Precursor | data |
| C20 | 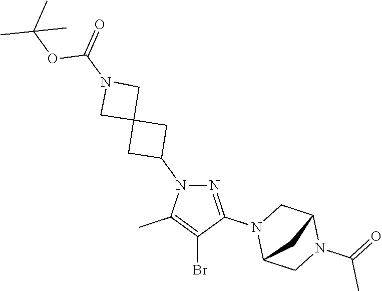 [SMILES: CC(=O)N1C[C@H]2C[C@@H]1CN2c1nn(C2CC3(C2)CN(C(=O)OC(C)(C)C)C3)c(C)c1Br] | Using Method-C19a from Intermediate A19 (Step 1) | UPLC-MS-1a: Rt = 1.01 min; MS m/z [M + H]+ 494.2/496.6. |
| | Tert-butyl 6-(3-((1S,4S)-5- | | |
| | acetyl-2,5- | | |
| | diazabicyclo[2.2.1]heptan-2- | | |
| | yl)-4-bromo-5-methyl-1H- | | |
| | pyrazol-1-yl)-2- | | |
| | azaspiro[3.3]heptane-2- | | |
| | carboxylate | | |
| C21 | 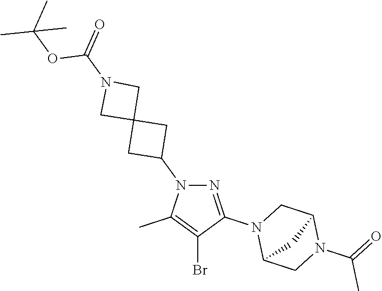 [SMILES: CC(=O)N1C[C@@H]2C[C@H]1CN2c1nn(C2CC3(C2)CN(C(=O)OC(C)(C)C)C3)c(C)c1Br] | Using Method-C19a from 1-((1S,4S)- 2,5-diazabicyclo [2.2.1]heptan-2-yl) ethan-1-one hydrochloride (CAS 1190927-48- 00) (Step 1) | UPLC-MS-1a: Rt = 1.01 min; MS m/z [M + H]+ 494.2/496.6 |
| | Tert-butyl 6-(3-((1S,4S)-5- | | |
| | acetyl-2,5- | | |
| | diazabicyclo[2.2.1]heptan-2- | | |
| | yl)-4-bromo-5-methyl-1H- | | |
| | pyrazol-1-yl)-2- | | |
| | azaspiro[3.3]heptane-2-carboxylate | | |
| C22 | 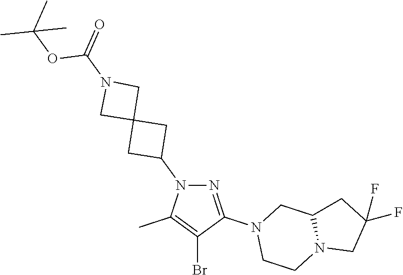 | Using Method-C19a from (8aS)-7,7- difluoro- octahydropyrrolo [1,2-a] pyrazine dihydrochloride (CAS 1305712-21-3) | UPLC-MS-1g: Rt = 1.21 min; MS m/z [M + H]+ 516.1/518.1. |
| | Tert-butyl (S)-6-(4-bromo-3- | | |
| | (7,7- | | |
| | difluorohexahydropyrrolo[1,2- | | |
| | a]pyrazin-2(1H)-yl)-5-methyl- | | |
| | 1H-pyrazol-1-yl)-2- | | |
| | azaspiro[3.3]heptane-2- | | |
| | carboxylate | | |

### Intermediates C23a and C23b: 1-(6-(3-(4-Acetyl-2-methylpiperazin-1-yl)-4-bromo-5-methyl-1H-pyrazol-1-yl)-2-azaspiro[3.3]heptan-2-yl)prop-2-en-1-one

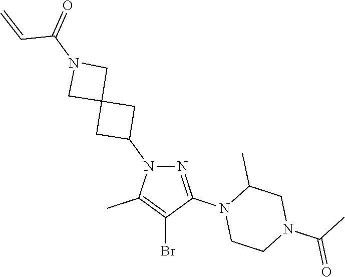 [SMILES: C=CC(=O)N1CC2(CC(n3nc(N4CCN(C(C)=O)CC4C)c(Br)c3C)C2)C1]

The title compound was prepared using analogous method to method-C19a (Step 1) and 2 starting form 1-(3-methylpiperazin-1-yl)ethan-1-one. The enantiomers were separated by chiral SFC (C-HPLC-19; mobile phase: n-heptane/EtOH/MeOH (85/7.5/7.5)+0.05% DEA) to give the first eluting enantiomer of the title compound: Intermediate C23a; C-HPLC-20 (mobile phase: heptane/[EtOH:MeOH (50:50)+0.05% DEA]: 85/15): Rt=2.84 min, UPLC-MS-1a: Rt=1.17 min; MS m/z [M+H]⁺ 496.3/498.3 and the second eluting isomer of the title compound: Intermediate C23b: C-HPLC-20 (mobile phase: heptane/[EtOH:MeOH (50:50)+0.05% DEA]: 85/15): Rt=4.59 min, UPLC-MS-1a: Rt=1.17 min; MS m/z [M+H]⁺ 496.3/498.3.

### Intermediates C24a and C24b: Tert-butyl 6-(4-bromo-5-methyl-3-(3-(pyridin-3-yl)pyrrolidin-1-yl)-1H-pyrazol-1-yl)-2-azaspiro[3.3]heptane-2-carboxylate

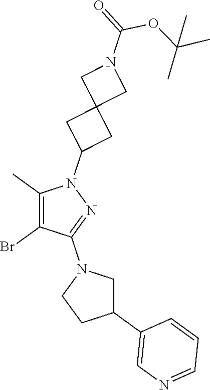 [SMILES: Cc1c(Br)c(N2CCC(c3cccnc3)C2)nn1C1CC2(C1)CN(C(=O)OC(C)(C)C)C2]

To a solution of tert-butyl 6-(4-bromo-3-iodo-5-methyl-1H-pyrazol-1-yl)-2-azaspiro[3.3]heptane-2-carboxylate (Intermediate C5, 500 mg, 1.04 mmol) in THF (6.90 mL) was added 3-(pyrrolidin-3-yl)pyridine (184 mg, 1.24 mmol), tBuXPhos-Pd-G3 (82 mg, 0.104 mmol) and then phosphazene base P₂Et [CAS [165535-45-5], 0.69 mL, 2.07 mmol). The reaction mixture was stirred at RT for 64 h. The RM was quenched with water, extracted twice with EtOAc. The combined organic layers were dried (Na₂SO₄), filtered and evaporated. The crude residue was purified by normal phase chromatography (eluent: MeOH in CH₂Cl₂ from 0 to 5%) and the enantiomers were separated by chiral SFC (C-SFC-2; mobile phase: CO₂/[CH₃OH+0.025% NH₃]: 70/30) to give the first eluting enantiomer of the title compound: Intermediate C24a; C-SFC-11 (mobile phase: CO₂/[CH₃OH+0.025% NH₃]: 70/30): Rt=2.34 min, UPLC-MS-1a: Rt=1.28 min; MS m/z [M+H]⁺; 502.2/504.2 and the second eluting enantiomer of the title compound: Intermediate C24b: C-SFC-11 (mobile phase: CO₂/[CH₃OH+0.025% NH₃]: 70/30): Rt=3.04 min, UPLC-MS-1a: Rt=1.28 min; MS m/z [M+H]⁺ 502.2/504.2.

### Intermediates C25a and C25b: Tert-butyl 6-(5-methyl-3-((3aS*,7aS*)-2-methyl-1-oxooctahydro-5H-pyrrolo[3,4-c]pyridin-5-yl)-1H-pyrazol-1-yl)-2-azaspiro[3.3]heptane-2-carboxylate

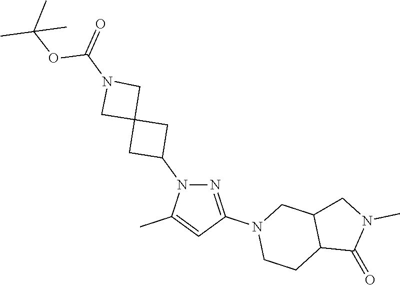 [SMILES: Cc1cc(N2CCC3C(=O)N(C)CC3C2)nn1C1CC2(C1)CN(C(=O)OC(C)(C)C)C2]

In an ACE tube, to a mixture of tert-butyl 6-(3-bromo-5-methyl-1H-pyrazol-1-yl)-2-azaspiro[3.3]heptane-2-carboxylate (Intermediate C1, 950 mg, 2.67 mmol), (3aS*,7aS*)-2-methyloctahydro-1H-pyrrolo[3,4-c]pyridin-1-one (Intermediate A58 racemic, 432 mg, 2.80 mmol), tBuXPhos-Pd-G3 (212 mg, 0.27 mmol) in THF (19 mL) was added Phosphazene P₂-Et (CAS [165535-45-5], 2.66 mL, 8.00 mmol). The reaction mixture was flushed with argon and stirred at 85° C. for 18 h. The reaction mixture was poured into water and extracted with CH₂Cl₂ (×3). The combined organic extracts were dried over (phase separator) and concentrated in vacuo. The crude residue was purified by normal phase chromatography (eluent: MeOH in CH₂Cl₂ from 0 to 5%) to give the title compound. The isomers were separated by chiral SFC (C-SFC-2; mobile phase: CO₂/[MeOH+0.025% NH₃] 85/15) to give Intermediate C25a as the first eluting peak: UPLC-MS-2a: Rt=0.98 min; MS m/z [M+H]⁺ 430.3, C-SFC-3 (mobile phase: CO₂/[MeOH+0.025% NH₃] 85/15): Rt=1.66 min and Intermediate C25b as the second eluting peak: UPLC-MS-2a: Rt=0.98 min; MS m/z [M+H]⁺ 430.3, C-SFC-3 (mobile phase: CO₂/[MeOH+0.025% NH₃] 85/15): Rt=2.54 min.

### Intermediates C26a and C26b: Tert-butyl 6-(4-iodo-5-methyl-3-((trans)-1-methyl-2-oxooctahydro-5H-pyrrolo[3,2-c]pyridin-5-yl)-1H-pyrazol-1-yl)-2-azaspiro[3.3]heptane-2-carboxylate

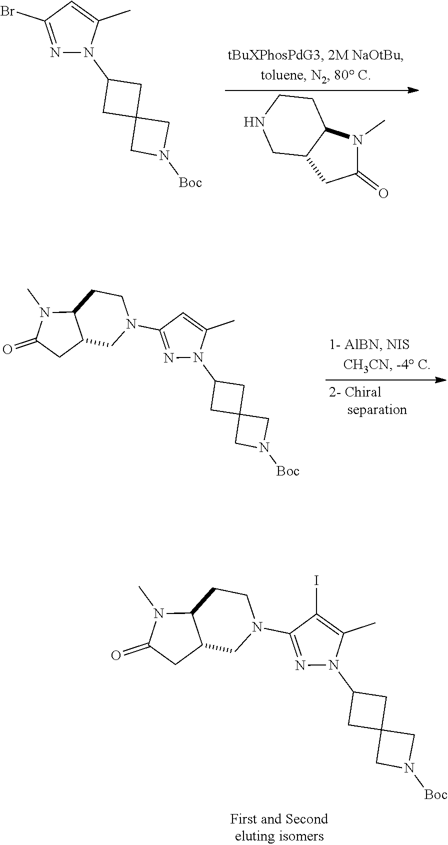 [SMILES: Cc1cc(Br)nn1C1CC2(C1)CN(C(=O)OC(C)(C)C)C2 ; CN1C(=O)C[C@@H]2CNCC[C@H]21 ; Cc1cc(N2CC[C@H]3[C@@H](CC(=O)N3C)C2)nn1C1CC2(C1)CN(C(=O)OC(C)(C)C)C2 ; Cc1c(I)c(N2CC[C@H]3[C@@H](CC(=O)N3C)C2)nn1C1CC2(C1)CN(C(=O)OC(C)(C)C)C2]

### Step 1: Tert-butyl 6-(5-methyl-3-((trans)-1-methyl-2-oxooctahydro-5H-pyrrolo[3,2-c]pyridin-5-yl)-1H-pyrazol-1-yl)-2-azaspiro[3.3]heptane-2-carboxylate

To a solution of (trans)-1-methyloctahydro-2H-pyrrolo[3,2-c]pyridin-2-one (Intermediate A59, 1.10 g, 6.80 mmol) and tert-butyl 6-(3-bromo-5-methyl-1H-pyrazol-1-yl)-2-azaspiro[3.3]heptane-2-carboxylate (Intermediate C1, 2.42 g, 6.80 mmol) in anhydrous toluene (190 mL) under nitrogen atmosphere was t-BuXPhos-Pd-G3 (0.81 g, 1.02 mmol) and the mixture was degassed with nitrogen for 5 min before the addition of NaOtBu (2M in THF, 10.7 mL, 21.4 mmol). The reaction mixture was stirred at 80° C. in a screw capped vial for 16 h. After completion of the reaction, the RM was diluted with EtOAc and filtered thorough a pad of celite, the filtrate was concentrated under vacuum and the crude residue was purified normal phase chromatography (eluent: 0 to 7% MeOH in CH₂Cl₂) to give the title product. UPLC-MS-5: Rt=1.62 min, MS m/z [M+H]⁺ 430.4.

### Step 2: Tert-butyl 6-(4-iodo-5-methyl-3-((trans)-1-methyl-2-oxooctahydro-5H-pyrrolo[3,2-c]pyridin-5-yl)-1H-pyrazol-1-yl)-2-azaspiro[3.3]heptane-2-carboxylate

To a solution of tert-butyl 6-(5-methyl-3-((trans)-1-methyl-2-oxooctahydro-5H-pyrrolo[3,2-c]pyridin-5-yl)-1H-pyrazol-1-yl)-2-azaspiro[3.3]heptane-2-carboxylate (Step 1, 1.97 g, 4.60 mmol) in CH₃CN (40 mL) was added at −4° C., AIBN (0.08 g, 0.460 mmol) followed by NIS (1.14 g, 5.05 mmol) and the reaction mixture was stirred at −4° C. for 2 h. After completion of reaction, the RM was quenched by adding a sat. aq. NaHCO₃ solution at −40° C. and the mixture was extracted with EtOAc. The combined organic layer was washed with brine, dried (Na₂SO₄), filtered and concentrated under vacuum. The crude residue was purified by normal phase chromatography (eluent: 0 to 7% MeOH in CH₂Cl₂) to give the title product. The isomers were separated by chiral SFC (C-SFC-2; mobile phase: CO₂/IPA 65/35) to give Intermediate C26a as the first eluting peak: UPLC-MS-2a: Rt=1.14 min; MS m/z [M+H]⁺ 556.1, C-SFC-3 (mobile phase: CO₂/IPA 65/35): Rt=1.00 min and Intermediate C26b and as the second eluting peak: UPLC-MS-2a: Rt=1.14 min; MS m/z [M+H]⁺ 556.1, C-SFC-3 (mobile phase: CO₂/IPA 65/35): Rt=2.34 min.

### Intermediates C27a and C27b: Tert-butyl 6-(4-iodo-5-methyl-3-(4,8,8-trimethyl-1-oxa-4,9-diazaspiro[5.5]undecan-9-yl)-1H-pyrazol-1-yl)-2-azaspiro[3.3]heptane-2-carboxylate

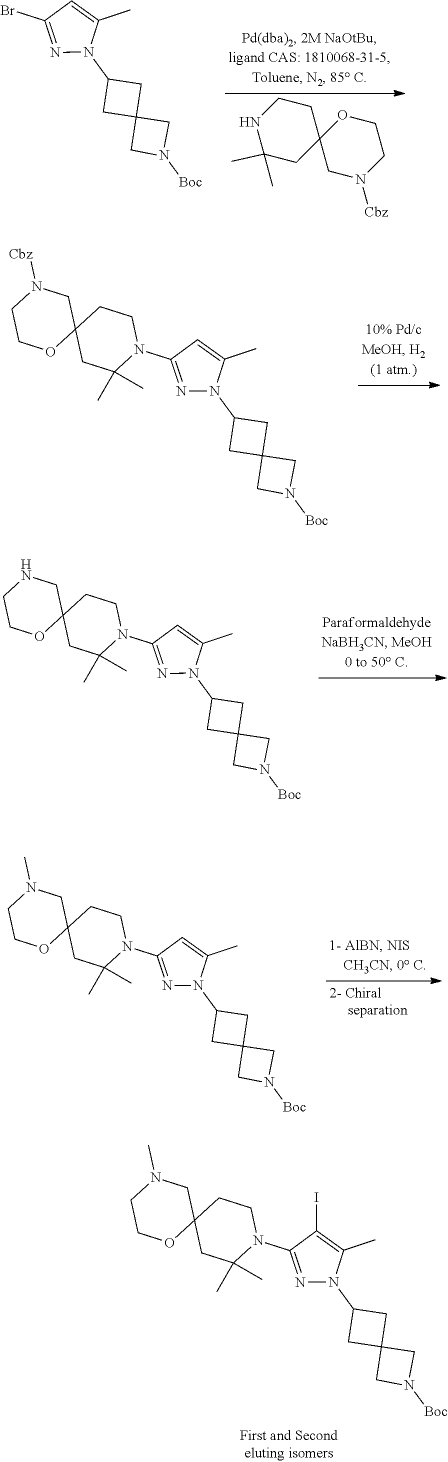 [SMILES: Cc1cc(Br)nn1C1CC2(C1)CN(C(=O)OC(C)(C)C)C2 ; CC1(C)CC2(CCN1)CN(C(=O)OCc1ccccc1)CCO2 ; Cc1cc(N2CCC3(CN(C(=O)OCc4ccccc4)CCO3)CC2(C)C)nn1C1CC2(C1)CN(C(=O)OC(C)(C)C)C2 ; Cc1cc(N2CCC3(CNCCO3)CC2(C)C)nn1C1CC2(C1)CN(C(=O)OC(C)(C)C)C2 ; Cc1cc(N2CCC3(CN(C)CCO3)CC2(C)C)nn1C1CC2(C1)CN(C(=O)OC(C)(C)C)C2 ; Cc1c(I)c(N2CCC3(CN(C)CCO3)CC2(C)C)nn1C1CC2(C1)CN(C(=O)OC(C)(C)C)C2]

### Step 1: Benzyl 9-(1-(2-(tert-butoxycarbonyl)-2-azaspiro[3.3]heptan-6-yl)-5-methyl-1H-pyrazol-3-yl)-8,8-dimethyl-1-oxa-4,9-diazaspiro[5.5]undecane-4-carboxylate

A solution of tert-butyl 6-(3-bromo-5-methyl-1H-pyrazol-1-yl)-2-azaspiro[3.3]heptane-2-carboxylate (Intermediate C1, 2.00 g, 5.61 mmol) and benzyl 8,8-dimethyl-1-oxa-4,9-diazaspiro[5.5]undecane-4-carboxylate (Intermediate A64, 2.32 g, 7.30 mmol) in toluene (50 mL) was degassed with nitrogen for 10 min and 2-[bis(3,5-trifluoromethylphenylphosphino)-3,6-dimethoxy]-2′,6′-di-i-propoxy-1,1′-biphenyl (CAS [1810068-31-5], 0.47 g, 0.61 mmol), Pd(dba)₂ (0.26 g, 0.46 mmol) followed by NaOtBu (2 M in THF, 5.05 mL, 10.1 mmol) were added sequentially and the reaction mixture was stirred at 85° C. for 3 h in a sealed tube. After completion of reaction, the RM was quenched with water, extracted with EtOAc (×2), the combined organic layer was washed with a sat. aq. NaHCO₃ solution, with brine, dried (Na₂SO₄), filtered and concentrated. The crude residue was purified by normal phase chromatography on neutral aluminum oxide (eluent: 0.5-1% MeOH in CH₂Cl₂) followed by reverse phase chromatography (eluent: 45% CH₃CN in H₂O containing 0.1% NH₃) to give the title compound. UPLC-MS-5: Rt=1.70 min; MS m/z [M+H]⁺ 594.7.

### Step 2: Tert-butyl 6-(3-(8,8-dimethyl-1-oxa-4,9-diazaspiro[5.5]undecan-9-yl)-5-methyl-1H-pyrazol-1-yl)-2-azaspiro[3.3]heptane-2-carboxylate

To a slurry of 10% Pd/C (1.20 g) in MeOH (20 mL) was added a solution of benzyl 9-(1-(2-(tert-butoxycarbonyl)-2-azaspiro[3.3]heptan-6-yl)-5-methyl-1H-pyrazol-3-yl)-8,8-dimethyl-1-oxa-4,9-diazaspiro[5.5]undecane-4-carboxylate (Step 1, 2.00 g, 3.36 mmol) in MeOH (40 mL) and the mixture was stirred at RT under hydrogen atmosphere for 3 h. After completion of the reaction, the RM was filtered through a pad of celite and washed with Methanol. The filtrate was concentrated under reduced pressure and the crude residue was purified by reverse phase chromatography (eluent: 60% CH₃CN in H₂O containing 0.025% NH₃) to give the title compound. UPLC-MS-5: Rt=1.38 min; MS m/z [M+H]⁺ 460.5.

### Step 3: Tert-butyl 6-(5-methyl-3-(4,8,8-trimethyl-1-oxa-4,9-diazaspiro[5.5]undecan-9-yl)-1H-pyrazol-1-yl)-2-azaspiro[3.3]heptane-2-carboxylate

To a solution of tert-butyl 6-(3-(8,8-dimethyl-1-oxa-4,9-diazaspiro[5.5]undecan-9-yl)-5-methyl-1H-pyrazol-1-yl)-2-azaspiro[3.3]heptane-2-carboxylate (Step 2, 1.35 g, 2.93 mmol) in MeOH (30 mL) was added paraformaldehyde (0.18 g, 5.87 mmol) and mixture was cooled to 0° C. NaBH₃CN (0.18 g, 2.93 mmol) was added portion wise and resulting reaction mixture was stirred at 50° C. for 1 h. After completion of the reaction, the RM was cooled to RT, filtered through a pad of celite and washed with EtOAc. The filtrate was concentrated under reduced pressure and the crude residue was purified by reverse phase chromatography (eluent: 60% CH₃CN in H₂O containing 0.025% NH₃) to give the title compound. UPLC-MS-5: Rt=1.43 min; MS m/z [M+H]⁺ 474.87.

### Step 4: Tert-butyl 6-(4-iodo-5-methyl-3-(4,8,8-trimethyl-1-oxa-4,9-diazaspiro[5.5]undecan-9-yl)-1H-pyrazol-1-yl)-2-azaspiro[3.3]heptane-2-carboxylate

To a solution of tert-butyl 6-(5-methyl-3-(4,8,8-trimethyl-1-oxa-4,9-diazaspiro[5.5]undecan-9-yl)-1H-pyrazol-1-yl)-2-azaspiro[3.3]heptane-2-carboxylate (Step 3, 1.20 g, 2.61 mmol) in CH₃CN (24 mL) at 0° C. under nitrogen atmosphere was added NIS (0.62 g, 2.74 mmol) portion wise and the reaction mixture was stirred at 0° C. for 10 min. After completion of the reaction, the RM was quenched with cold water and extracted with CH₂Cl₂. The combined organic layer was washed with brine, dried (Na₂SO₄), filtered and concentrated. The crude residue was purified by reverse phase chromatography (eluent: 80% CH₃CN in H₂O containing 0.025% NH₃) to give the title compound. The enantiomers were separated by chiral SFC (C-SFC-47; mobile phase: CO₂/[IPA/CH₃CN (50/50)+0.1% Et₂NH] 85/15) to give Intermediate C27a as the first eluting enantiomer: UPLC-MS-5: Rt=1.69 min; MS m/z [M+H]⁺ 600.3, C-SFC-48 (mobile phase: CO₂/[IPA/CH₃CN (50/50)+0.1% Et₂NH]85/15): Rt=2.57 min and Intermediate C27b as the second eluting enantiomer: UPLC-MS-5: Rt=1.69 min; MS m/z [M+H]⁺ 600.3, C-SFC-48 (mobile phase: CO₂/[IPA/CH₃CN (50/50)+0.1% Et₂NH]85/15): Rt=3.46 min.

### Intermediate C28: Tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate

### Method-C28-A

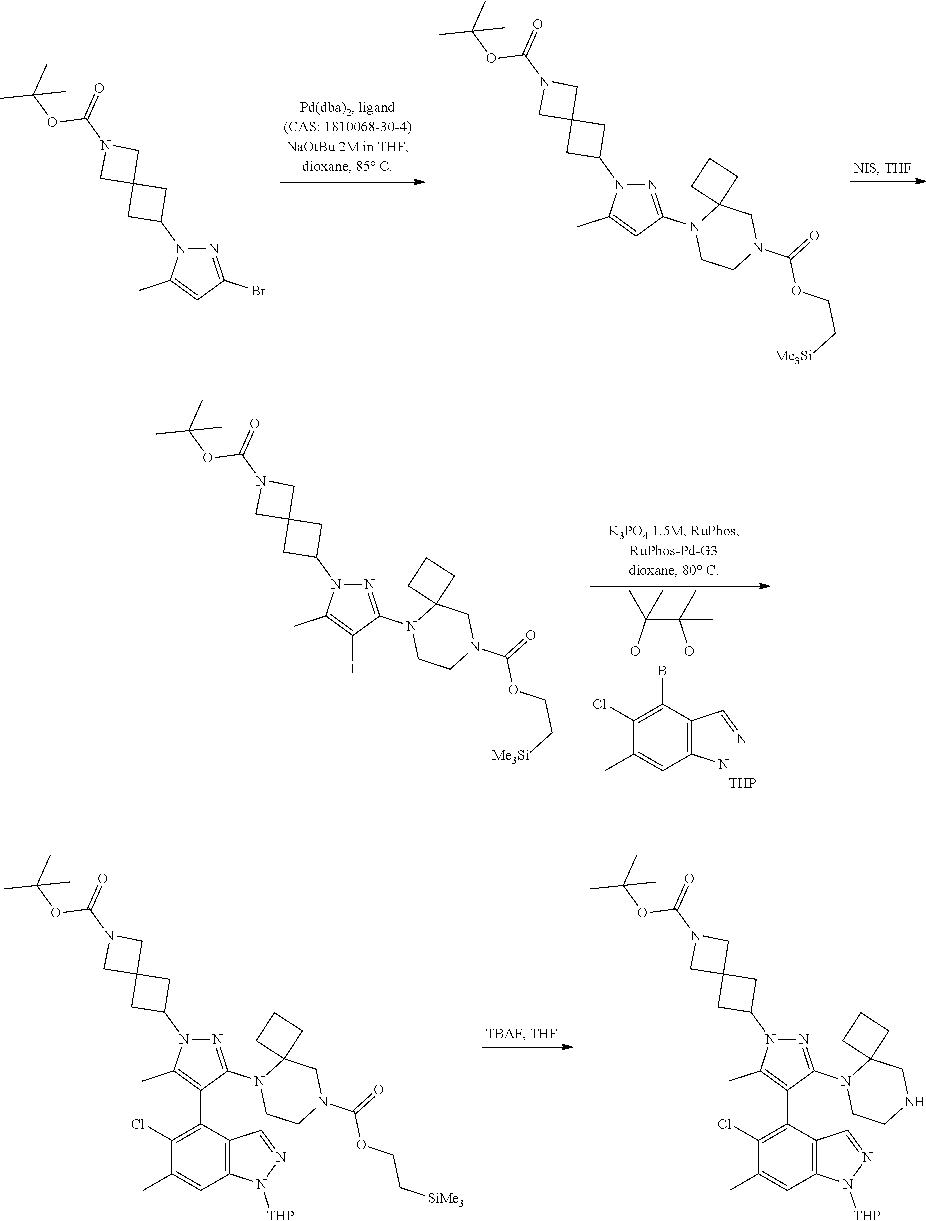

### Step 1: 2-(Trimethylsilyl)ethyl 5-(1-(2-(tert-butoxycarbonyl)-2-azaspiro[3.3]heptan-6-yl)-5-methyl-1H-pyrazol-3-yl)-5,8-diazaspiro[3.5]nonane-8-carboxylate

Under inert atmosphere, to a stirred solution of tert-butyl 6-(3-bromo-5-methyl-1H-pyrazol-1-yl)-2-azaspiro[3.3]heptane-2-carboxylate (Intermediate C1, 11.0 g, 30.9 mmol), 2-(trimethylsilyl)ethyl 5,8-diazaspiro[3.5]nonane-8-carboxylate (Intermediate A30, 9.19 g, 34.0 mmol), bis(3,5-bis(trifluoromethyl)phenyl)(2′,6′-bis(dimethylamino)-3,6-dimethoxybiphenyl-2-yl)phosphine (CAS: [1810068-30-4], 1.19 g, 1.58 mmol), and Pd(dba)₂ (888 mg, 1.54 mmol) in 1,4-dioxane (100 mL) was added NaOtBu (2M solution in THF, 21.6 mL, 43.2 mmol). The reaction mixture was placed in a preheated oil bath (85° C.) and stirred at 85° C. for 2 h. The RM was quenched with a sat. aq. NaHCO₃ solution and extracted with EtOAc (×2). The combined organic layers were washed with a sat. aq. NaHCO₃ solution, dried (Na₂SO₄), filtered and concentrated under vacuum. The crude residue was purified by normal phase chromatography (eluent: EtOAc in cyclohexane 0 to 47%) to give the title compound as a brown solid. UPLC-MS-2a: Rt=1.40 min; MS m/z [M+H]⁺ 546.3.

### Step 2: 2-(Trimethylsilyl)ethyl 5-(1-(2-(tert-butoxycarbonyl)-2-azaspiro[3.3]heptan-6-yl)-4-iodo-5-methyl-1H-pyrazol-3-yl)-5,8-diazaspiro[3.5]nonane-8-carboxylate

Under inert atmosphere, to a stirred solution of 2-(trimethylsilyl)ethyl 5-(1-(2-(tert-butoxycarbonyl)-2-azaspiro[3.3]heptan-6-yl)-5-methyl-1H-pyrazol-3-yl)-5,8-diazaspiro[3.5]nonane-8-carboxylate (Step 1, 17.0 g, 31.1 mmol) in THF (150 mL) was added NIS (7.36 g, 32.7 mmol) at 0° C. and the reaction mixture was stirred at RT for 16 h. The reaction was quenched with a sat. aq. NaHCO₃ solution and extracted with EtOAc (×2). The combined organic layers were washed with a sat. aq. NaHCO₃ solution, dried (Na₂SO₄), filtered and concentrated under vacuum. The crude residue was purified by normal phase chromatography (eluent: EtOAc in c-hexane 0 to 37%) to give the title compound as a white solid. UPLC-MS-2a: Rt=1.49 min; MS m/z [M+H]⁺ 672.3.

### Step 3: 2-(Trimethylsilyl)ethyl 5-(1-(2-(tert-butoxycarbonyl)-2-azaspiro[3.3]heptan-6-yl)-4-(5-chloro-6-methyl-1-(tetrahydro-2H-pyran-2-yl)-1H-indazol-4-yl)-5-methyl-1H-pyrazol-3-yl)-5,8-diazaspiro[3.5]nonane-8-carboxylate

Under inert atmosphere, to a stirred solution of 2-(trimethylsilyl)ethyl 5-(1-(2-(tert-butoxycarbonyl)-2-azaspiro[3.3]heptan-6-yl)-4-iodo-5-methyl-1H-pyrazol-3-yl)-5,8-diazaspiro[3.5]nonane-8-carboxylate (Step 2, 20.5 g, 30.5 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 13.8 g, 36.6 mmol) and K₃PO₄ (19.4 g, 91.0 mmol) in a mixture of 1,4-dioxane (100 mL) and water (25.0 mL) were added RuPhos (1.42 g, 3.05 mmol) and RuPhos-Pd-G3 (2.55 g, 3.05 mmol). The reaction mixture placed in a preheated oil batch (80° C.) and was stirred at 80° C. for 1 h. The RM was quenched with a sat. aq. NaHCO₃ solution and extracted with EtOAc (×2). The combined organic layers were washed with a sat. aq. NaHCO₃ solution, dried (Na₂SO₄), filtered and concentrated under vacuum. The residue was taken up in CH₂Cl₂, Si-TMT (CAS [1226494-16-1], 3.50 mmol) was added and the mixture was swirled at 40° C. for 1 h. The mixture was filtered and the filtrate was concentrated under vacuum. The crude residue was purified by normal phase chromatography (eluent: EtOAc in c-hexane 0 to 47%) to give the title compound as a yellow solid. UPLC-MS-2a: Rt=1.48 min; MS m/z [M+H]⁺ 794.6.

### Step 4: Tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate

Under inert atmosphere, to a stirred solution of 2-(trimethylsilyl)ethyl 5-(1-(2-(tert-butoxycarbonyl)-2-azaspiro[3.3]heptan-6-yl)-4-(5-chloro-6-methyl-1-(tetrahydro-2H-pyran-2-yl)-1H-indazol-4-yl)-5-methyl-1H-pyrazol-3-yl)-5,8-diazaspiro[3.5]nonane-8-carboxylate (Step 3, 26.8 g, 33.7 mmol) in THF (150 mL) was added TBAF (1M in THF, 84.0 mL, 84.0 mmol). The reaction mixture was stirred at RT for 20 h. The RM was quenched with a sat. aq. NH₄Cl solution and extracted with EtOAc (×2). The combined organic layers were washed with a sat. aq. NaHCO₃ solution, dried (Na₂SO₄), filtered and concentrated under vacuum. The crude residue was purified by normal phase chromatography (eluent: EtOAc 100% then MeOH in CH₂Cl₂ 0 to 10%) to give the title compound as a yellow solid. UPLC-MS-2a: Rt=1.08 min; MS m/z [M+H]⁺ 650.5/652.5.

### Method-C28-B

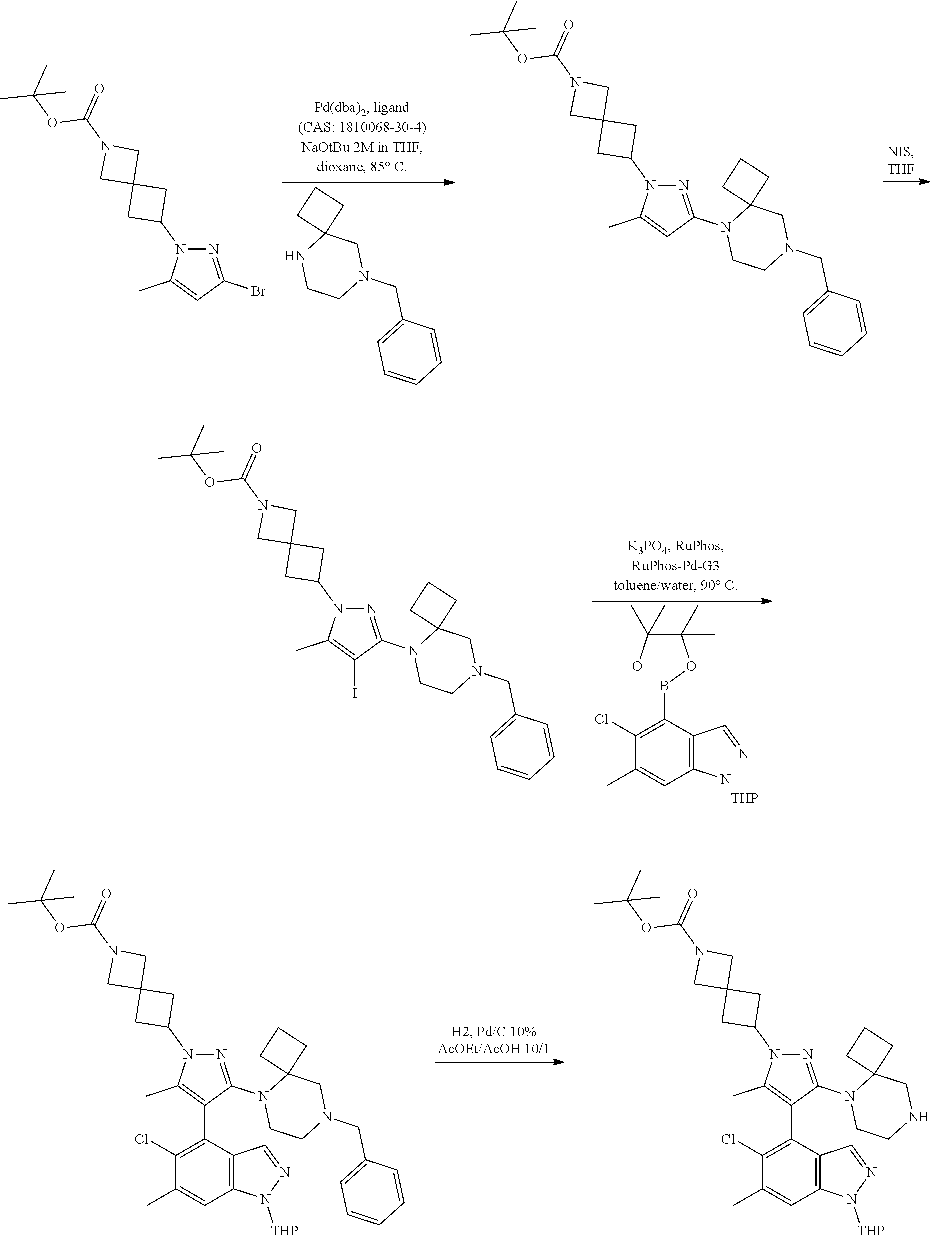

### Step 1: Tert-butyl 6-(3-(8-benzyl-5,8-diazaspiro[3.5]nonan-5-yl)-5-methyl-1H-pyrazol-1-yl)-2-azaspiro[3.3]heptane-2-carboxylate

The title compound was prepared by a method similar to Method-C28-A (Step 1) using 8-benzyl-5,8-diazaspiro[3.5]nonane (Intermediate A31) instead of 2-(trimethylsilyl)ethyl 5,8-diazaspiro[3.5]nonane-8-carboxylate (Intermediate A30). The title compound was obtained as a brown gum after normal phase chromatography (eluent: EtOAc in n-heptane 0 to 60%). UPLC-MS-2a: Rt=0.96 min; MS m/z [M+H]⁺ 492.4.

### Step 2: Tert-butyl 6-(3-(8-benzyl-5,8-diazaspiro[3.5]nonan-5-yl)-4-iodo-5-methyl-1H-pyrazol-1-yl)-2-azaspiro[3.3]heptane-2-carboxylate

The title compound was prepared by a method similar to Method-C28-A (Step 2) by replacing 2-(trimethylsilyl)ethyl 5-(1-(2-(tert-butoxycarbonyl)-2-azaspiro[3.3]heptan-6-yl)-5-methyl-1H-pyrazol-3-yl)-5,8-diazaspiro[3.5]nonane-8-carboxylate with tert-butyl 6-(3-(8-benzyl-5,8-diazaspiro[3.5]nonan-5-yl)-5-methyl-1H-pyrazol-1-yl)-2-azaspiro[3.3]heptane-2-carboxylate (Protocol B, Step 1). The title compound was obtained as a white foam after normal phase chromatography (eluent: EtOAc in n-heptane 0 to 60%). UPLC-MS-2a: Rt 1.06 min; MS m/z [M+H]⁺ 618.3.

### Step 3: Tert-butyl 6-(3-(8-benzyl-5,8-diazaspiro[3.5]nonan-5-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate

The title compound was prepared by a method similar to Method-C28-A (Step 3) by replacing 2-(trimethylsilyl)ethyl 5-(1-(2-(tert-butoxycarbonyl)-2-azaspiro[3.3]heptan-6-yl)-4-iodo-5-methyl-1H-pyrazol-3-yl)-5,8-diazaspiro[3.5]nonane-8-carboxylate with tert-butyl 6-(3-(8-benzyl-5,8-diazaspiro[3.5]nonan-5-yl)-4-iodo-5-methyl-1H-pyrazol-1-yl)-2-azaspiro[3.3]heptane-2-carboxylate (Protocol B, Step 2). The title compound was obtained as a white foam after normal phase chromatography (eluent: EtOAc in n-heptane 0 to 50%). UPLC-MS-2a: Rt 1.16 min; MS m/z [M+H]⁺ 740.5/742.5.

### Step 4: Tert-butyl 6-(4-(5-chloro-6-methyl-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate

Tert-butyl 6-(3-(8-benzyl-5,8-diazaspiro[3.5]nonan-5-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Step 3, 100 mg, 0.11 mmol) was dissolved in EtOAc:AcOH 10:1 (4.40 mL) and 10% Pd/C (23 mg) was added. The reaction mixture was placed under a pressure of hydrogen (5 bars) and stirred at RT for 20 h. The RM was filtered through a celite pad, washed with EtOAc, and the filtrate was washed twice with a sat. aq. NaHCO₃ solution then with brine. The separated organic layer was dried (Na₂SO₄), filtered, and concentrated under vacuum. The crude residue was purified by normal phase chromatography (eluent: MeOH in CH₂Cl₂ 0 to 12%) to give the title compound. UPLC-MS-2a: Rt=1.08 min; MS m/z [M+H]⁺ 650.5/652.5.

### Intermediate C29: Tert-butyl 6-(3-(8-(2-hydroxy-2-methylpropyl)-5,8-diazaspiro[3.5]nonan-5-yl)-5-methyl-1H-pyrazol-1-yl)-2-azaspiro[3.3]heptane-2-carboxylate

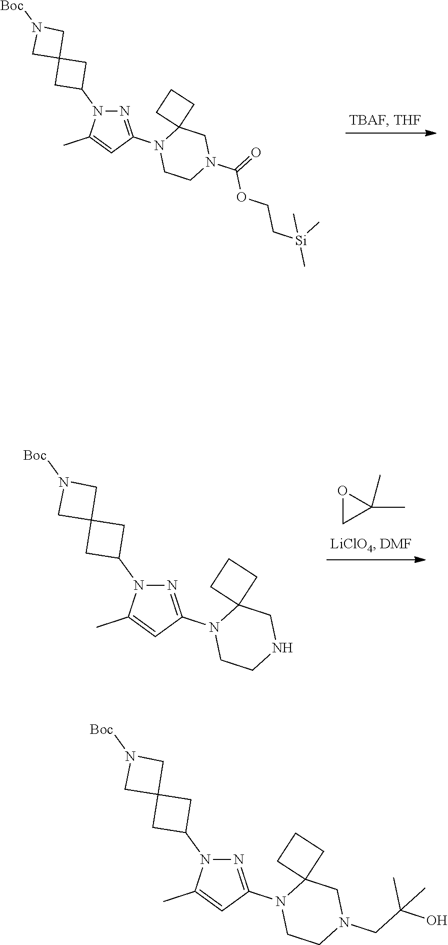

### Step 1: Tert-butyl 6-(5-methyl-3-(5,8-diazaspiro[3.5]nonan-5-yl)-1H-pyrazol-1-yl)-2-azaspiro[3.3]heptane-2-carboxylate

TBAF (8.54 mL, 8.54 mmol) was added to a solution of 2-(trimethylsilyl)ethyl 5-(1-(2-(tert-butoxycarbonyl)-2-azaspiro[3.3]heptan-6-yl)-5-methyl-1H-pyrazol-3-yl)-5,8-diazaspiro[3.5]nonane-8-carboxylate (Intermediate prepared in Method-C28-A (Step 1), 2.05 g, 3.42 mmol) in THF (11 mL). After stirring at RT overnight, the reaction mixture was filtered and concentrated under reduced pressure. The residue was diluted with EtOAc, washed with a sat. aq. NaHCO₃ solution and brine, dried (Na₂SO₄) and concentrated under reduced pressure. The crude residue was purified by normal phase chromatography (eluent: EtOAc in c-hexane 0 to 30%) to give the title compound. UPLC-MS-3: Rt=0.71 min; MS m/z [M+H]⁺ 402.4.

### Step 2: Tert-butyl 6-(3-(8-(2-hydroxy-2-methylpropyl)-5,8-diazaspiro[3.5]nonan-5-yl)-5-methyl-1H-pyrazol-1-yl)-2-azaspiro[3.3]heptane-2-carboxylate

LiClO₄ (1.76 g, 16.6 mmol) was added to a solution of tert-butyl 6-(5-methyl-3-(5,8-diazaspiro[3.5]nonan-5-yl)-1H-pyrazol-1-yl)-2-azaspiro[3.3]heptane-2-carboxylate (Step 1, 900 mg, 1.66 mmol) and 2,2-dimethyloxirane (2.96 mL, 33.2 mmol) in DMF (12 mL). After stirring for 1 h at 60° C., the reaction mixture was filtered and concentrated under reduced pressure. The residue was diluted with EtOAc, washed with a sat. aq. NaHCO₃ solution and brine, dried (Na₂SO₄), filtered and concentrated under reduced pressure. The crude residue was purified by normal phase chromatography (eluent: EtOAc in c-hexane 0 to 30%) to give the title compound. UPLC-MS-3: Rt=0.75 min; MS m/z [M+H]⁺ 474.4.

### Intermediate C30: Tert-butyl 6-(4-(5-chloro-6-methyl-1-(tetrahydro-2H-pyran-2-yl)-1H-indazol-4-yl)-3-(2,2-diethylpiperazin-1-yl)-5-methyl-1H-pyrazol-1-yl)-2-azaspiro[3.3]heptane-2-carboxylate

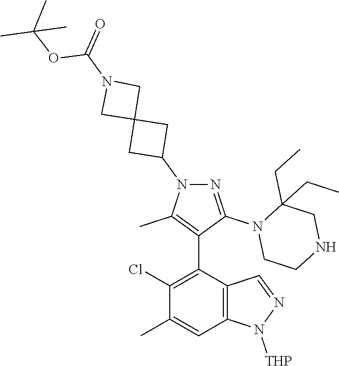

The title compound was prepared by a method similar to Method-C28-A using 2-(trimethylsilyl)ethyl 3,3-diethylpiperazine-1-carboxylate (intermediate A40) instead of 2-(trimethylsilyl)ethyl 5,8-diazaspiro[3.5]nonane-8-carboxylate (intermediate A30) in Step 1. UPLC-MS-2a: Rt=1.08 min; MS m/z [M+H]⁺ 666.6/668.6.

### Intermediate C31: Tert-butyl 6-(4-(5-chloro-6-methyl-1-(tetrahydro-2H-pyran-2-yl)-1H-indazol-4-yl)-5-methyl-3-(6,9-diazaspiro[4.5]decan-6-yl)-1H-pyrazol-1-yl)-2-azaspiro[3.3]heptane-2-carboxylate

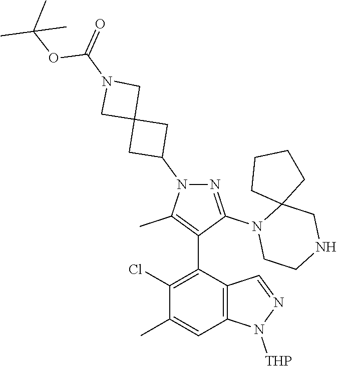

The title compound was prepared by a method similar to Method-C28-A except that Step 3 was performed in toluene instead of dioxane using 2-(trimethylsilyl)ethyl 6,9-diazaspiro[4.5]decane-9-carboxylate (intermediate A41) instead of 2-(trimethylsilyl)ethyl 5,8-diazaspiro[3.5]nonane-8-carboxylate (intermediate A30) in Step 1. UPLC-MS-3: Rt=1.07 min; MS m/z [M+H]⁺ 664.4/666.5.

### Intermediate C32: Tert-butyl 6-(4-(5-chloro-6-fluoro-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate

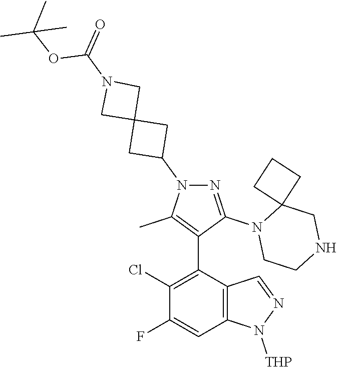

The title compound was prepared by a method similar to Method-C28-A using 5-chloro-6-fluoro-1-(tetrahydro-2H-pyran-2-yl)-4-(4,4,5,5-tetramethyl-1,3,2-dioxaborolan-2-yl)-1H-indazole (Intermediate D9) instead of 5-chloro-6-methyl-1-(tetrahydro-2H-pyran-2-yl)-4-(4,4,5,5-tetramethyl-1,3,2-dioxaborolan-2-yl)-1H-indazole (Intermediate D1) in Step 3. UPLC-MS-2a: Rt=1.07 min; MS m/z [M+H]⁺: 654.4/656.5.

### Intermediate C33: Tert-butyl 6-(4-(5,6-dichloro-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate

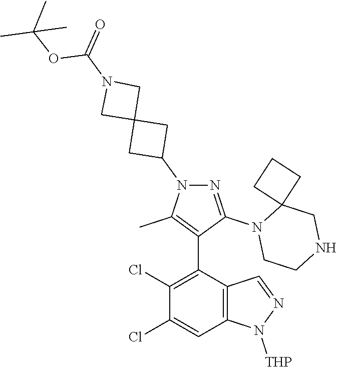

The title compound was prepared by a method similar to Method-C28-A using 5,6-dichloro-1-(tetrahydro-2H-pyran-2-yl)-4-(4,4,5,5-tetramethyl-1,3,2-dioxaborolan-2-yl)-1H-indazole (intermediate D6) instead of 5-chloro-6-methyl-1-(tetrahydro-2H-pyran-2-yl)-4-(4,4,5,5-tetramethyl-1,3,2-dioxaborolan-2-yl)-1H-indazole (Intermediate D1) in Step 3. UPLC-MS-2a: Rt=1.11 min; MS m/z [M+H]⁺: 670.5/672.4/674.5.

### Intermediate C34: Tert-butyl 6-(4-(5,6-dichloro-1-(tetrahydro-2H-pyran-2-yl)-1H-indazol-4-yl)-3-(2,2-dimethylpiperazin-1-yl)-5-methyl-1H-pyrazol-1-yl)-2-azaspiro[3.3]heptane-2-carboxylate

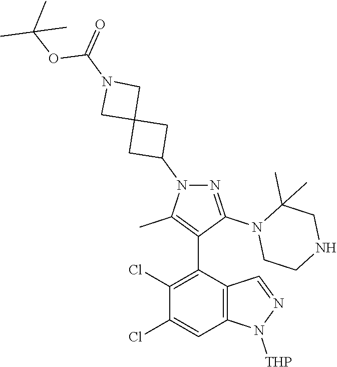

The title compound was prepared by a method similar to Method-C28-A from 2-(trimethylsilyl)ethyl 3,3-dimethylpiperazine-1-carboxylate (intermediate A43) and using 5,6-dichloro-1-(tetrahydro-2H-pyran-2-yl)-4-(4,4,5,5-tetramethyl-1,3,2-dioxaborolan-2-yl)-1H-indazole (intermediate D6) instead of intermediate D1 in Step 3 and heating the reaction mixture at 40° C. in Step 4. UPLC-MS-4: Rt=1.13 min; MS m/z [M+H]⁺ 658.3/660.3/662.3.

### Intermediate C35: Tert-butyl 6-(4-(5,6-dichloro-1-(tetrahydro-2H-pyran-2-yl)-1H-indazol-4-yl)-3-((S)-2-ethyl-2-methylpiperazin-1-yl)-5-methyl-1H-pyrazol-1-yl)-2-azaspiro[3.3]heptane-2-carboxylate

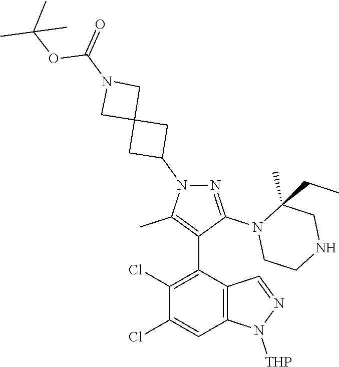

The title compound was prepared by a method similar to Method-C28-A (Step 2-4) from tert-butyl (S)-6-(3-(2-ethyl-2-methyl-4-((2-(trimethylsilyl)ethoxy)carbonyl)piperazin-1-yl)-5-methyl-1H-pyrazol-1-yl)-2-azaspiro[3.3]heptane-2-carboxylate (intermediate C67) and using 5,6-dichloro-1-(tetrahydro-2H-pyran-2-yl)-4-(4,4,5,5-tetramethyl-1,3,2-dioxaborolan-2-yl)-1H-indazole (intermediate D6) instead of intermediate D1 in Step 3. Step 3 was carried out using tetrakis(triphenylphosphine)palladium and the reaction mixture was stirred at 80° C. for 4 h. UPLC-MS-4: Rt=1.12 min; MS m/z [M+H]⁺ 672.5/674.4/676.5.

### Intermediate C36a and C36b: Tert-butyl 6-(4-(5-chloro-6-methyl-1-(tetrahydro-2H-pyran-2-yl)-1H-indazol-4-yl)-3-((4aS*,7aS*)-hexahydrofuro[3,4-b]pyrazin-1(2H)-yl)-5-methyl-1H-pyrazol-1-yl)-2-azaspiro[3.3]heptane-2-carboxylate

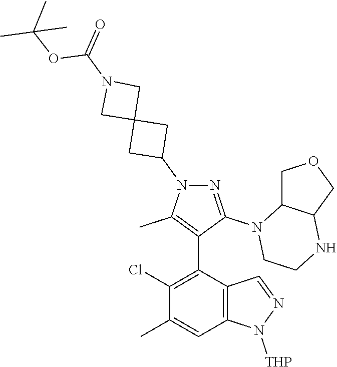

Intermediate C36a: Tert-butyl 6-(4-(5-chloro-6-methyl-1-(tetrahydro-2H-pyran-2-yl)-1H-indazol-4-yl)-3-((4aS*,7aS*)-hexahydrofuro[3,4-b]pyrazin-1(2H)-yl)-5-methyl-1H-pyrazol-1-yl)-2-azaspiro[3.3]heptane-2-carboxylate Isomer 1 The title compound was prepared by a method similar to Method-C28-A (Step 2-4) using 2-(trimethylsilyl)ethyl (4aS*,7aS*)-4-(1-(2-(tert-butoxycarbonyl)-2-azaspiro[3.3]heptan-6-yl)-5-methyl-1H-pyrazol-3-yl)hexahydrofuro[3,4-b]pyrazine-1 (2H)-carboxylate Isomer 1 (described below). UPLC-MS-2a: Rt=1.04 min; MS m/z [M+H]⁺ 652.5/654.5.

### Intermediate C36b: Tert-butyl 6-(4-(5-chloro-6-methyl-1-(tetrahydro-2H-pyran-2-yl)-1H-indazol-4-yl)-3-((4aS*,7aS*)-hexahydrofuro[3,4-b]pyrazin-1(2H)-yl)-5-methyl-1H-pyrazol-1-yl)-2-azaspiro[3.3]heptane-2-carboxylate Isomer 2

The title compound was prepared by a method similar to Method-C28-A (Step 2-4) using 2-(trimethylsilyl)ethyl (4aS*,7aS*)-4-(1-(2-(tert-butoxycarbonyl)-2-azaspiro[3.3]heptan-6-yl)-5-methyl-1H-pyrazol-3-yl)hexahydrofuro[3,4-b]pyrazine-1(2H)-carboxylate Isomer 2 (described below). UPLC-MS-3: Rt=1.04 min; MS m/z [M+H]⁺ 652.2/654.4.

### 2-(Trimethylsilyl)ethyl (4aS*,7aS*)-4-(1-(2-(tert-butoxycarbonyl)-2-azaspiro[3.3]heptan-6-yl)-5-methyl-1H-pyrazol-3-yl)hexahydrofuro[3,4-b]pyrazine-1(2H)-carboxylate Isomer 1 and Isomer 2

The title compound was prepared by a method similar to Method-C28-A (Step 1) using 2-(trimethylsilyl)ethyl (4aS*,7aS*)-hexahydrofuro[3,4-b]pyrazine-1(2H)-carboxylate (Intermediate A42) instead of 2-(trimethylsilyl)ethyl 5,8-diazaspiro[3.5]nonane-8-carboxylate (Intermediate A30). The isomers were separated by chiral SFC (C-SFC-5; mobile phase: CO₂/MeOH: 85/15) to give the first eluting isomer of the title compound: Isomer 1: C-SFC-6 (mobile phase: CO₂/MeOH: 85/15): Rt=1.20 min, UPLC-MS-2a: Rt=1.31 min; MS m/z [M+H]⁺ 548.9 and the second eluting isomer of the title compound: Isomer 2: C-SFC-6 (mobile phase: CO₂/MeOH: 85/15): Rt=1.69 min, UPLC-MS-2a: Rt=1.31 min; MS m/z [M+H]⁺ 548.4.

### Intermediate C37: Tert-Butyl 6-(4-(6-chloro-5-methoxy-1-(tetrahydro-2H-pyran-2-yl)-1H-indazol-4-yl)-5-methyl-3-(5,8-diazaspiro[3.5]nonan-5-yl)-1H-pyrazol-1-yl)-2-azaspiro[3.3]heptane-2-carboxylate

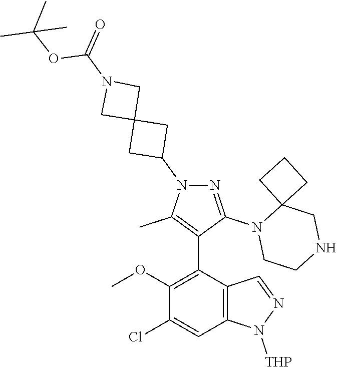

The title compound was prepared by a method similar to Method-C28-A using 6-chloro-5-methoxy-1-(tetrahydro-2H-pyran-2-yl)-4-(4,4,5,5-tetramethyl-1,3,2-dioxaborolan-2-yl)-1H-indazole (Intermediate D8) instead of 5-chloro-6-methyl-1-(tetrahydro-2H-pyran-2-yl)-4-(4,4,5,5-tetramethyl-1,3,2-dioxaborolan-2-yl)-1H-indazole (Intermediate D1) in Step 3. UPLC-MS-2a: Rt=1.07/1.08 min; MS m/z [M+H]⁺ 666.4/668.4.

### Intermediate C38: Tert-butyl 6-(4-(5-chloro-6-methyl-1-(tetrahydro-2H-pyran-2-yl)-1H-indazol-4-yl)-3-(2,2-dimethylpiperazin-1-yl)-5-methyl-1H-pyrazol-1-yl)-2-azaspiro[3.3]heptane-2-carboxylate

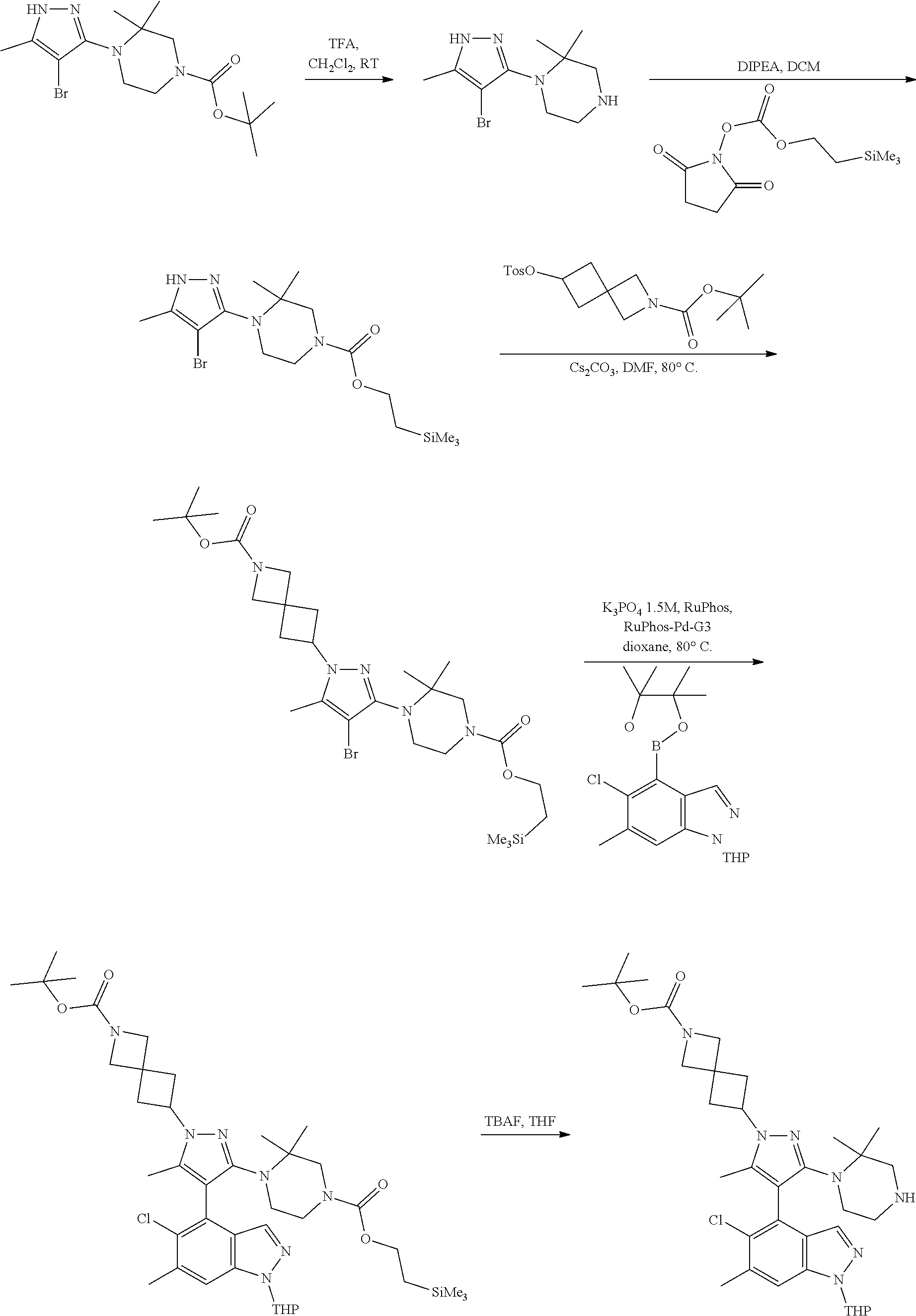

### Step 1: 1-(4-Bromo-5-methyl-1H-pyrazol-3-yl)-2,2-dimethylpiperazine

To a solution of tert-butyl 4-(4-bromo-5-methyl-1H-pyrazol-3-yl)-3,3-dimethylpiperazine-1-carboxylate (Intermediate C11, 324 g, 832 mmol) in CH₂Cl₂ (7.77 L) was added TFA (950 g, 8.30 mol) at 0° C. After completion of the reaction, the reaction mixture was concentrated under vacuum to give the title compound as a trifluoroacetate salt which was used without purification in the next step. UPLC-MS-1a: Rt=0.50 min; MS m/z [M+H]⁺ 273.0/275.0.

### Step 2: 2-(Trimethylsilyl)ethyl 4-(4-bromo-5-methyl-1H-pyrazol-3-yl)-3,3-dimethylpiperazine-1-carboxylate

To a solution of 1-(4-bromo-5-methyl-1H-pyrazol-3-yl)-2,2-dimethylpiperazine trifluoroacetate salt (Step 1, 9.02 mmol) in CH₂Cl₂ (20.0 mL) were added DIPEA (9.46 mL, 54.1 mmol) and 2,5-dioxopyrrolidin-1-yl (2-(trimethylsilyl)ethyl) carbonate (2.81 g, 10.8 mmol) and the reaction mixture was stirred at RT overnight. The RM was partitioned between brine and EtOAc and the aqueous layer was extracted with EtOAc (×2). The combined organic layers were dried (phase separator) and the filtrate was concentrated under vacuum. The residue was purified by normal phase chromatography (eluent: MeOH in CH₂Cl₂ 0 to 20%) to give the title compound as a white foam. UPLC-MS-2a: Rt=1.32 min; MS m/z [M+H]⁺ 417.1/419.1.

### Step 3: Tert-butyl 6-(4-bromo-3-(2,2-dimethyl-4-((2-(trimethylsilyl)ethoxy)carbonyl)piperazin-1-yl)-5-methyl-1H-pyrazol-1-yl)-2-azaspiro[3.3]heptane-2-carboxylate

To a solution of 2-(trimethylsilyl)ethyl 4-(4-bromo-5-methyl-1H-pyrazol-3-yl)-3,3-dimethylpiperazine-1-carboxylate (Step 2, 6.50 g, 14.0 mmol) in dry DMF (70.0 mL) were added tert-butyl 6-(tosyloxy)-2-azaspiro[3.3]heptane-2-carboxylate (Intermediate C2, 5.15 g, 14.0 mmol) and Cs₂CO₃ (11.4 g, 35 mmol) at RT. The reaction mixture was stirred at 80° C. overnight. The RM was diluted with water and extracted twice with EtOAc. The combined organic layers were washed with brine, dried (Na₂SO₄), and the filtrate was concentrated under vacuum. The crude residue was purified by normal phase chromatography (eluent: EtOAc in c-hexane 0 to 100%) to give the title compound as a white foam. UPLC-MS-2a: Rt=1.47 min; MS m/z [M+H]⁺ 612.2/614.2.

### Step 4: Tert-butyl 6-(4-(5-chloro-6-methyl-1-(tetrahydro-2H-pyran-2-yl)-1H-indazol-4-yl)-3-(2,2-dimethyl-4-((2-(trimethylsilyl)ethoxy)carbonyl)piperazin-1-yl)-5-methyl-1H-pyrazol-1-yl)-2-azaspiro[3.3]heptane-2-carboxylate

The title compound was prepared by a method similar to Method-C28-A (Step 3) by replacing 2-(trimethylsilyl)ethyl 5-(1-(2-(tert-butoxycarbonyl)-2-azaspiro[3.3]heptan-6-yl)-4-iodo-5-methyl-1H-pyrazol-3-yl)-5,8-diazaspiro[3.5]nonane-8-carboxylate with tert-butyl 6-(4-bromo-3-(2,2-dimethyl-4-((2-(trimethylsilyl)ethoxy)carbonyl)piperazin-1-yl)-5-methyl-1H-pyrazol-1-yl)-2-azaspiro[3.3]heptane-2-carboxylate (Step 3). The title compound was obtained as a yellow foam after normal phase chromatography (eluent: EtOAc in c-hexane 0 to 100%). UPLC-MS-2a: Rt=1.46 min; MS m/z [M+H]⁺ 782.5/784.5.

### Step 5: Tert-butyl 6-(4-(5-chloro-6-methyl-1-(tetrahydro-2H-pyran-2-yl)-1H-indazol-4-yl)-3-(2,2-dimethylpiperazin-1-yl)-5-methyl-1H-pyrazol-1-yl)-2-azaspiro[3.3]heptane-2-carboxylate

The title compound was prepared by a method similar to Method-C28-A (Step 4) by replacing 2-(trimethylsilyl)ethyl 5-(1-(2-(tert-butoxycarbonyl)-2-azaspiro[3.3]heptan-6-yl)-4-(5-chloro-6-methyl-1-(tetrahydro-2H-pyran-2-yl)-1H-indazol-4-yl)-5-methyl-1H-pyrazol-3-yl)-5,8-diazaspiro[3.5]nonane-8-carboxylate with tert-butyl 6-(4-(5-chloro-6-methyl-1-(tetrahydro-2H-pyran-2-yl)-1H-indazol-4-yl)-3-(2,2-dimethyl-4-((2-(trimethylsilyl)ethoxy)carbonyl)piperazin-1-yl)-5-methyl-1H-pyrazol-1-yl)-2-azaspiro[3.3]heptane-2-carboxylate (Step 4). The title compound obtained as a yellow foam and was used without purification in the next step. ¹H NMR (600 MHz, DMSO-d₆) δ 7.68 (s, 1H), 7.62 (d, 1H), 5.85-5.75 (m, 1H), 4.78-4.67 (m, 1H), 4.01-3.85 (m, 5H), 3.80-3.70 (m, 1H), 3.63-3.58 (m, 2H), 2.93-2.86 (m, 2H), 2.73-2.61 (m, 4H), 2.51 (s, 3H), 2.45 (t, 2H), 2.40-2.37 (m, 1H), 2.07-1.91 (m, 5H), 1.77-1.72 (m, 2H), 1.62-1.55 (m, 2H), 1.38 (s, 9H), 1.07 (d, 3H), 0.99 (d, 3H). UPLC-MS-2a: Rt=1.06 min; MS m/z [M+H]⁺ 638.3.

### Method-C39 for the preparation of Intermediate C39: Tert-butyl 6-(4-(5-chloro-6-methyl-1-(tetrahydro-2H-pyran-2-yl)-1H-indazol-4-yl)-3-((S)-2-ethyl-2-methylpiperazin-1-yl)-5-methyl-1H-pyrazol-1-yl)-2-azaspiro[3.3]heptane-2-carboxylate

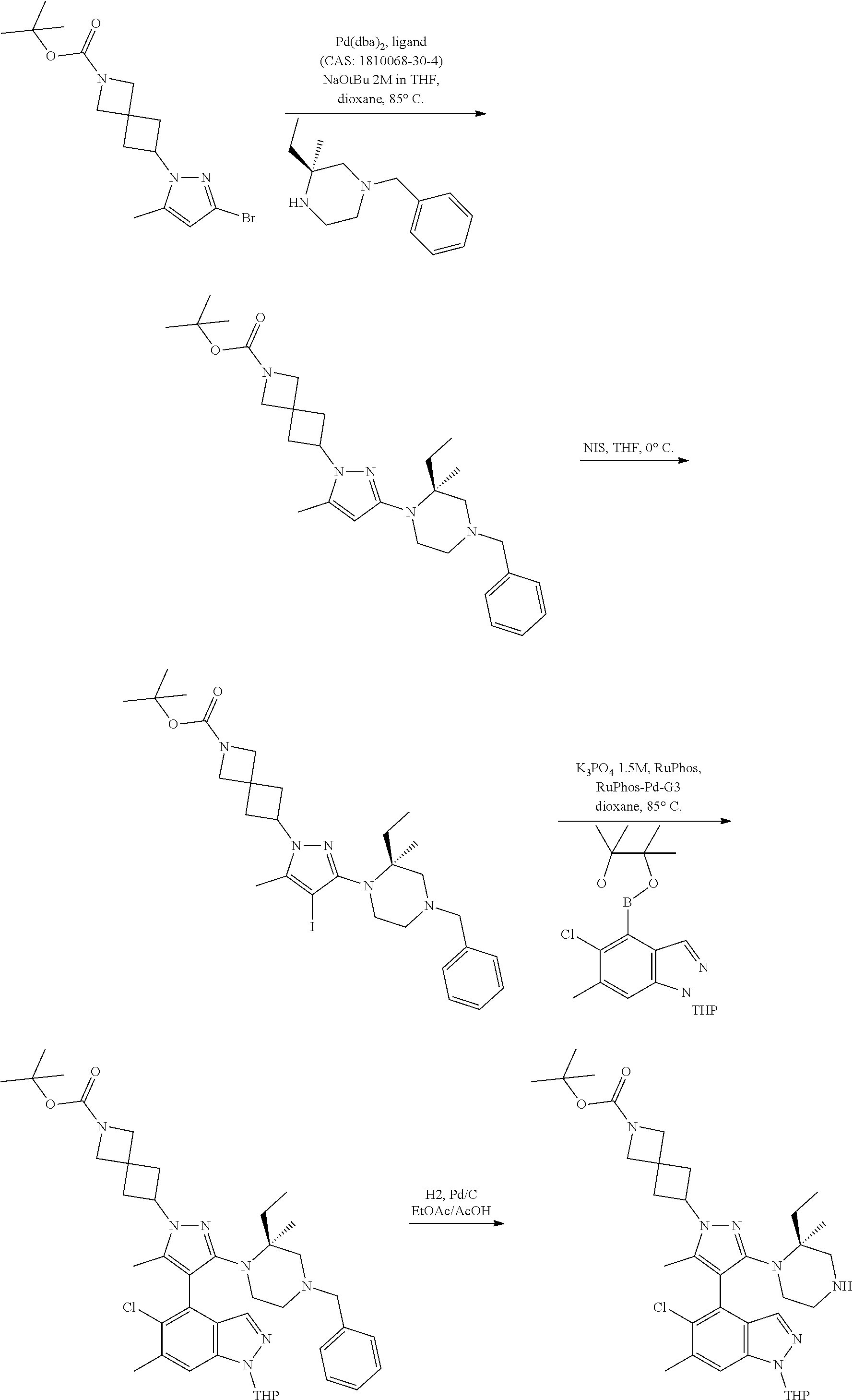

### Step 1: Tert-butyl (S)-6-(3-(4-benzyl-2-ethyl-2-methylpiperazin-1-yl)-5-methyl-1H-pyrazol-1-yl)-2-azaspiro[3.3]heptane-2-carboxylate

Under inert atmosphere, to a stirred solution of tert-butyl 6-(3-bromo-5-methyl-1H-pyrazol-1-yl)-2-azaspiro[3.3]heptane-2-carboxylate (Intermediate C1, 20.0 g, 56.1 mmol), (S)-1-benzyl-3-ethyl-3-methylpiperazine (Intermediate A32, 15.3 g, 70.2 mmol), bis(3,5-bis(trifluoromethyl)phenyl)(2′,6′-bis(dimethylamino)-3,6-dimethoxybiphenyl-2-yl)phosphine (CAS: 1810068-30-4, 3.19 g, 4.21 mmol), and Pd(dba)₂ (2.41 g, 4.21 mmol) in 1,4-dioxane (300 mL) was added NaOtBu (2M in THF, 42.1 mL, 84.0 mmol). The reaction mixture was placed in a preheated bath (85° C.) and stirred at 85° C. for 16 h. After cooling to RT, the reaction mixture was poured into saturated aqueous NaHCO₃ solution and extracted with EtOAc. The combined organic layers were washed with brine, dried (Na₂SO₄), filtered and concentrated. The crude residue was purified by normal phase chromatography (eluent: [EtOAc/MeOH 20/1] in n-heptane 0 to 100%) to give the title compound as an orange oil. UPLC-MS-4: Rt=1.01 min; MS m/z [M+H]⁺ 494.4.

### Step 2: Tert-butyl (S)-6-(3-(4-benzyl-2-ethyl-2-methylpiperazin-1-yl)-4-iodo-5-methyl-1H-pyrazol-1-yl)-2-azaspiro[3.3]heptane-2-carboxylate

To an ice-cooled solution of tert-butyl (S)-6-(3-(4-benzyl-2-ethyl-2-methylpiperazin-1-yl)-5-methyl-1H-pyrazol-1-yl)-2-azaspiro[3.3]heptane-2-carboxylate (Step 1, 19.2 g, 36.9 mmol) in THF (185 mL) was added NIS (9.14 mg, 40.6 mmol) at 0° C. and the reaction mixture was stirred at RT for 80 min. The RM was poured into EtOAc/water and extracted with EtOAc. The combined organic layers were washed with a sat. aq. Na₂S₂O₃ solution then brine, dried (Na₂SO₄), filtered, and concentrated under vacuum. The crude residue was purified by normal phase chromatography (eluent: [EtOAc/MeOH 20/1] in n-heptane 0 to 50%) to give the title compound as a yellow foam. UPLC-MS-2a: Rt=1.11 min; MS m/z [M+H]⁺ 620.4.

### Step 3: Tert-butyl 6-(3-((S)-4-benzyl-2-ethyl-2-methylpiperazin-1-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate

Under inert atmosphere, to a stirred solution of tert-butyl (S)-6-(3-(4-benzyl-2-ethyl-2-methylpiperazin-1-yl)-4-iodo-5-methyl-1H-pyrazol-1-yl)-2-azaspiro[3.3]heptane-2-carboxylate (Step 2, 19.2 g, 30.0 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 16.9 g, 45.0 mmol), and K₃PO₄ (2M aq. Solution, 45.0 mL, 90.0 mmol) in 1,4-dioxane (300 mL) was added RuPhos (1.47 g, 3.00 mmol) and RuPhos-Pd-G3 (2.56 g, 3.00 mmol) and the reaction mixture was stirred at 85° C. for 1 h. The RM was quenched with NaHCO₃ (1M aq. Solution) and extracted with EtOAc (×2). The combined organic layers were washed with brine, dried (Na₂SO₄) and filtered. To the filtrate was added SiliaMetS®Thiol (4.8 mmol) and the mixture was swirled at 40° C. for 10 min. The mixture was filtered and concentrated under vacuum. The crude residue was purified by normal phase chromatography (eluent: [EtOAc/MeOH 20/1] in n-heptane 0 to 40%) to give the title compound as an orange foam. UPLC-MS-2a: Rt=1.15 min; MS m/z [M+H]⁺ 742.6.

### Step 4: Tert-butyl 6-(4-(5-chloro-6-methyl-1-(tetrahydro-2H-pyran-2-yl)-1H-indazol-4-yl)-3-((S)-2-ethyl-2-methylpiperazin-1-yl)-5-methyl-1H-pyrazol-1-yl)-2-azaspiro[3.3]heptane-2-carboxylate

Tert-butyl 6-(3-((S)-4-benzyl-2-ethyl-2-methylpiperazin-1-yl)-4-(5-chloro-6-methyl-1-(tetrahydro-2H-pyran-2-yl)-1H-indazol-4-yl)-5-methyl-1H-pyrazol-1-yl)-2-azaspiro[3.3]heptane-2-carboxylate (Step 3, 19.8 g, 25.3 mmol) was dissolved in a mixture of EtOAc (253 mL) and AcOH (14.5 mL) and 10% Pd/C (2.70 g) was added. The reaction mixture was placed under a pressure of hydrogen (3 bars) and stirred at RT for 20 h. The RM was filtered through a celite pad and the filtrate was poured into NaHCO₃ (1M aq. Solution), then extracted with EtOAc (×2). The combined organic layers were washed with NaHCO₃ (1M aq. Solution), dried (Na₂SO₄), filtered, and concentrated under vacuum. The crude residue was purified by normal phase chromatography (eluent: [CH₂Cl₂/MeOH/Et₃N 200/20/2] in CH₂Cl₂ 0 to 100%) to give the title compound as off-white foam. ¹H NMR (400 MHz, DMSO-d₆) δ 8.65 (br, 1H), 7.71 (s, 0.8H), 7.65 (s, 0.2H), 7.60-7.64 (m, 0.8H), 7.56-7.60 (m, 0.2H), 5.74-5.85 (m, 1H), 4.62-4.79 (m, 1H), 3.81-4.04 (m, 5H), 3.74 (m, 1H), 3.00-3.21 (m, 2H), 2.85 (m, 1H), 2.55-2.78 (m, 6H), 2.49 (s, 3H), 2.30-2.44 (m, 1H), 1.88-2.07 (m, 5H), 1.62-1.80 (m, 2H), 1.53-1.61 (m, 2H), 1.38 (s, 6.9H), 1.37 (m, 2.1H), 0.90 (m, 1.6H), 0.83 (m, 1.4H), 0.49 (m, 1.6H), 0.39 (m, 1.4H). UPLC-MS-2a: Rt=1.07 ml; MS m/z [M+H]⁺ 652.5/654.5.

Method-C39a: similar to Method-C39 except that Step 4 was performed with 2 equivalents of DIPEA in hexafluoroisopropanol as solvent instead of EtOAc/AcOH.

The following intermediates C40 to C48 were prepared using analogous methods to Method-C29 from intermediates described in the intermediates synthesis section or commercially available (in Step 1, 2 or 3).

| Intermediate | Structure | Precursor | Characterizing data |
|---|---|---|---|
| C40 | 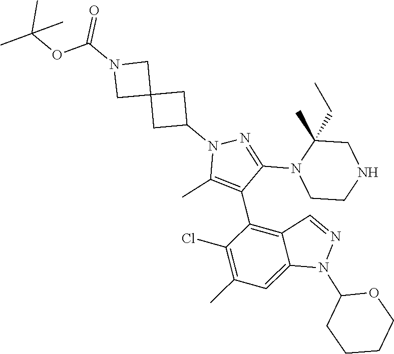 [SMILES: CC[C@]1(C)CNCCN1c1nn(C2CC3(C2)CN(C(=O)OC(C)(C)C)C3)c(C)c1-c1c(Cl)c(C)cc2c1cnn2C1CCCCO1] | Using Method-C39 from Intermediate A33 (Step 1) | UPLC-MS-2a: Rt = 1.13 min; MS m/z [M + H]⁺ 652.5/654.5 |
| | Tert-butyl 6-(4-(5-chloro-6- | | |
| | methyl-1-(tetrahydro-2H- | | |
| | pyran-2-yl)-1H-indazol-4-yl)-3- | | |
| | ((R)-2-ethyl-2-methylpiperazin- | | |
| | 1-yl)-5-methyl-1H-pyrazol-1- | | |
| | yl)-2-azaspiro[3.3]heptane-2- | | |
| | carboxylate | | |
| C41 | 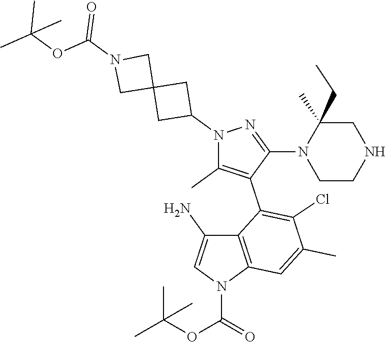 [SMILES: CC[C@@]1(C)CNCCN1c1nn(C2CC3(C2)CN(C(=O)OC(C)(C)C)C3)c(C)c1-c1c(Cl)c(C)cc2c1c(N)cn2C(=O)OC(C)(C)C] | Using Method-C39 from Intermediate A32 (Step 1) and Intermediate D11 (Step 3) | tert-butyl 3-amino-5- chloro-6-methyl-4- (4,4,5,5-tetramethyl- 1,3,2-dioxaborolan-2- yl)-1H-indazole-1- carboxylate instead of Intermediate D1. UPLC-MS-4: Rt = 1.09/1.12 min; MS m/z [M + H]+ 683.6/685.6. |
| | Tert-Butyl (S)-3-amino-4-(1-(2- | | |
| | (tert-butoxycarbonyl)-2- | | |
| | azaspiro[3.3]heptan-6-yl)-3-(2- | | |
| | ethyl-2-methylpiperazin-1-yl)- | | |
| | 5-methyl-1H-pyrazol-4-yl)-5- | | |
| | chloro-6-methyl-1H-indazole- | | |
| | 1-carboxylate | | |
| C42 | 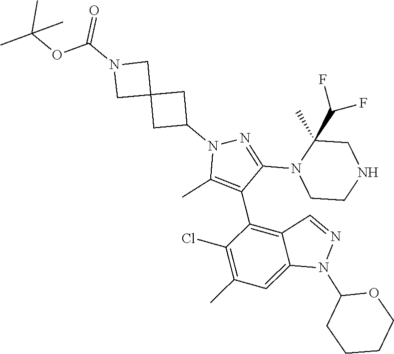 [SMILES: Cc1cc2c(cnn2C2CCCCO2)c(-c2c(N3CCNC[C@]3(C)C(F)F)nn(C3CC4(C3)CN(C(=O)OC(C)(C)C)C4)c2C)c1Cl] | Using Method- C39a from Intermediate A34 (Step 2) | UPLC-MS-2a: Rt = 1.04 min; MS m/z [M + H]⁺ 674.5/676.4 |
| | Tert-butyl 6-(4-(5-chloro-6- | | |
| | methyl-1-(tetrahydro-2H- | | |
| | pyran-2-yl)-1H-indazol-4-yl)-3- | | |
| | ((R)-2-(difluoromethyl)-2- | | |
| | methylpiperazin-1-yl)-5- | | |
| | methyl-1H-pyrazol-1-yl)-2- | | |
| | azaspiro[3.3]heptane-2- | | |
| | carboxylate | | |
| C43 | 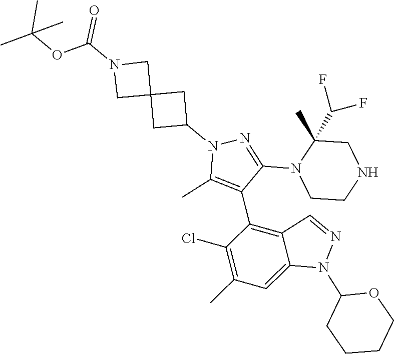 [SMILES: Cc1cc2c(cnn2C2CCCCO2)c(-c2c(N3CCNC[C@@]3(C)C(F)F)nn(C3CC4(C3)CN(C(=O)OC(C)(C)C)C4)c2C)c1Cl] | Using Method-C39 a from Intermediate A35 (Step 2) | UPLC-MS-2a: Rt = 1.04 min; MS m/z [M + H]⁺ 674.5/676.4. |
| | Tert-butyl 6-(4-(5-chloro-6- | | |
| | methyl-1-(tetrahydro-2H- | | |
| | pyran-2-yl)-1H-indazol-4-yl)-3- | | |
| | ((S)-2-(difluoromethyl)-2- | | |
| | methylpiperazin-1-yl)-5- | | |
| | methyl-1H-pyrazol-1-yl)-2- | | |
| | azaspiro[3.3]heptane-2- | | |
| | carboxylate | | |
| C44 | 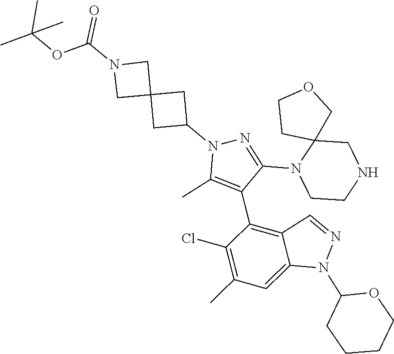 [SMILES: Cc1cc2c(cnn2C2CCCCO2)c(-c2c(N3CCNCC34CCOC4)nn(C3CC4(C3)CN(C(=O)OC(C)(C)C)C4)c2C)c1Cl] | Using Method-C39 from Intermediate C49a (Step 2) | UPLC-MS-4: Rt = 1.06 and 1.07 min; MS m/z [M + H]⁺ 666.4/668.4. |
| | Tert-butyl 6-(4-(5-chloro-6- | | |
| | methyl-1-(tetrahydro-2H- | | |
| | pyran-2-yl)-1H-indazol-4-yl)-5- | | |
| | methyl-3-(2-oxa-6,9- | | |
| | diazaspiro[4.5]decan-6-yl)-1H- | | |
| | pyrazol-1-yl)-2- | | |
| | azaspiro[3.3]heptane-2- | | |
| | carboxylate | | |
| C45 | 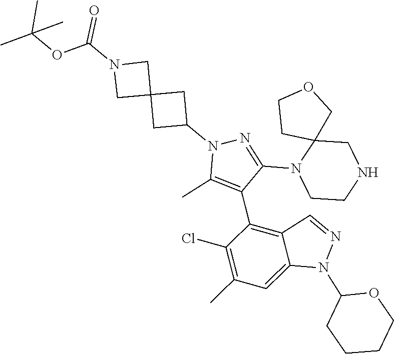 [SMILES: Cc1cc2c(cnn2C2CCCCO2)c(-c2c(N3CCNCC34CCOC4)nn(C3CC4(C3)CN(C(=O)OC(C)(C)C)C4)c2C)c1Cl] | Using Method-C39 from Intermediate C49b (Step 2) | UPLC-MS-4: Rt = 1.05 and 1.06 min; MS m/z [M + H]⁺ 666.4/668.4. |
| | Tert-butyl 6-(4-(5-chloro-6- | | |
| | methyl-1-(tetrahydro-2H- | | |
| | pyran-2-yl)-1H-indazol-4-yl)-5- | | |
| | methyl-3-(2-oxa-6,9- | | |
| | diazaspiro[4.5]decan-6-yl)-1H- | | |
| | pyrazol-1-yl)-2- | | |
| | azaspiro[3.3]heptane-2- | | |
| | carboxylate | | |
| C46 | 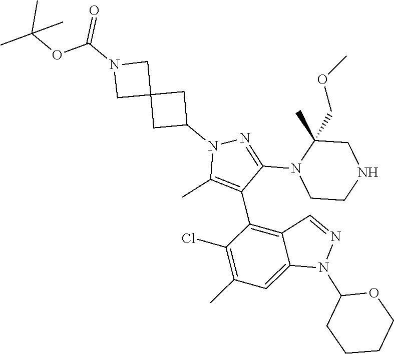 [SMILES: COC[C@]1(C)CNCCN1c1nn(C2CC3(C2)CN(C(=O)OC(C)(C)C)C3)c(C)c1-c1c(Cl)c(C)cc2c1cnn2C1CCCCO1] | Using Method-C39 from Intermediate C50a (Step 3) | UPLC-MS-4: Rt = 1.10 min; MS m/z [M + H]⁺ 668.6/670.7. |
| | Tert-butyl 6-(4-(5-chloro-6- | | |
| | methyl-1-(tetrahydro-2H- | | |
| | pyran-2-yl)-1H-indazol-4-yl)-3- | | |
| | ((S)-2-(methoxymethyl)-2- | | |
| | methylpiperazin-1-yl)-5- | | |
| | methyl-1H-pyrazol-1-yl)-2- | | |
| | azaspiro[3.3]heptane-2- | | |
| | carboxylate | | |
| C47 | 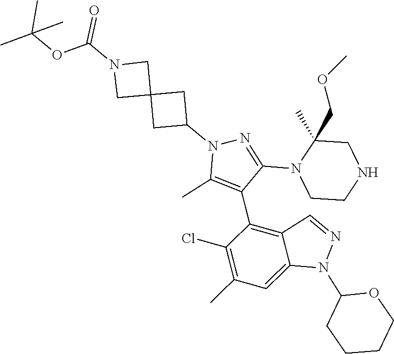 [SMILES: COC[C@@]1(C)CNCCN1c1nn(C2CC3(C2)CN(C(=O)OC(C)(C)C)C3)c(C)c1-c1c(Cl)c(C)cc2c1cnn2C1CCCCO1] | Using Method-C39 from Intermediate A37 (Step 1) or from Intermediate C50b (Step 3) | UPLC-MS-4: Rt = 1.10 min; MS m/z [M + H]⁺ 668.6/670.7. |
| | Tert-butyl 6-(4-(5-chloro-6- | | |
| | methyl-1-(tetrahydro-2H- | | |
| | pyran-2-yl)-1H-indazol-4-yl)-3- | | |
| | ((R)-2-(methoxymethyl)-2- | | |
| | methylpiperazin-1-yl)-5- | | |
| | methyl-1H-pyrazol-1-yl)-2- | | |
| | azaspiro[3.3]heptane-2- | | |
| | carboxylate | | |
| C48 | 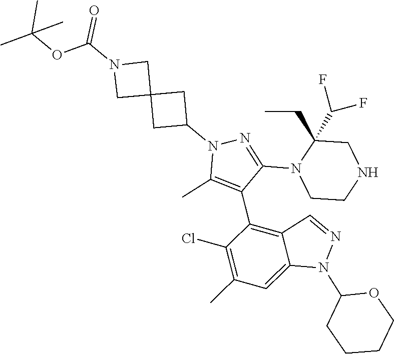 [SMILES: CC[C@@]1(C(F)F)CNCCN1c1nn(C2CC3(C2)CN(C(=O)OC(C)(C)C)C3)c(C)c1-c1c(Cl)c(C)cc2c1cnn2C1CCCCO1] | Using Method-C39 from Intermediate A36 (Step 1) | UPLC-MS-4: Rt = 1.11 min; MS m/z [M + H]⁺ 688.5/690.5. |
| | Tert-butyl 6-(4-(5-chloro-6- | | |
| | methyl-1-(tetrahydro-2H- | | |
| | pyran-2-yl)-1H-indazol-4-yl)-3- | | |
| | ((S)-2-(difluoromethyl)-2- | | |
| | ethylpiperazin-1-yl)-5-methyl- | | |
| | 1H-pyrazol-1-yl)-2- | | |
| | azaspiro[3.3]heptane-2- | | |
| | carboxylate | | |

### Intermediate C49a and C49b: Tert-butyl 6-(3-(9-benzyl-2-oxa-6,9-diazaspiro[4.5]decan-6-yl)-5-methyl-1H-pyrazol-1-yl)-2-azaspiro[3.3]heptane-2-carboxylate Isomer 1 and Isomer 2

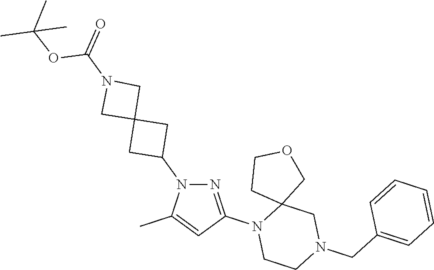 [SMILES: Cc1cc(N2CCN(Cc3ccccc3)CC23CCOC3)nn1C1CC2(C1)CN(C(=O)OC(C)(C)C)C2]

The title compound was prepared by a method similar to Step 1 of Method-C39 using 9-benzyl-2-oxa-6,9-diazaspiro[4.5]decane (Intermediate A44) instead of (S)-1-benzyl-3-ethyl-3-methylpiperazine (Intermediate A32). The enantiomers were separated by chiral SFC (C-SFC-2: mobile phase: CO₂/[IPA+0.025% NH₃]: 82/18) to give the first eluting enantiomer of the title compound: Intermediate C49a; C-SFC-3 (mobile phase: CO₂/[IPA+0.025% NH₃]: 83/17): Rt=2.31 min, UPLC-MS-3: Rt=0.96 min; MS m/z [M+H]⁺ 508.4 and the second eluting enantiomer of the title compound: Intermediate C49b: C-SFC-3 (mobile phase: CO₂/[IPA+0.025% NH₃]: 83/17): Rt=2.84 min, UPLC-MS-3: Rt=0.96 min; MS m/z [M+H]⁺ 508.4.

### Intermediates C50a and C50b: Tert-butyl (S)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate (C50a) and Tert-butyl (R)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate (C50b)

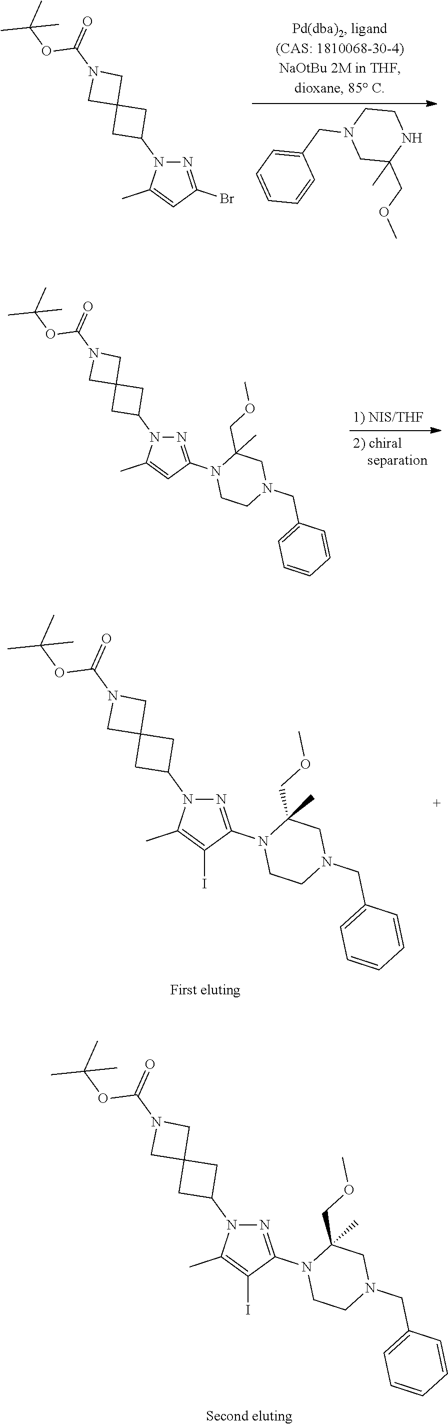

### Step 1: Tert-butyl 6-(3-(4-benzyl-2-(methoxymethyl)-2-methylpiperazin-1-yl)-5-methyl-1H-pyrazol-1-yl)-2-azaspiro[3.3]heptane-2-carboxylate

The title compound was prepared by a method similar to Method-C39 (Step 1) using 1-benzyl-3-(methoxymethyl)-3-methylpiperazine (Intermediate A37-rac) instead of (S)-1-benzyl-3-ethyl-3-methylpiperazine (Intermediate A32). UPLC-MS-4: Rt=0.91 min; MS m/z [M+H]⁺ 510.4.

### Step 2: Tert-butyl (R)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate and Tert-butyl (S)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate

The title compound was prepared by a method similar to Method-C39 (Step 2) using tert-butyl 6-(3-(4-benzyl-2-(methoxymethyl)-2-methylpiperazin-1-yl)-5-methyl-1H-pyrazol-1-yl)-2-azaspiro[3.3]heptane-2-carboxylate (Step 1) instead of tert-butyl (S)-6-(3-(4-benzyl-2-ethyl-2-methylpiperazin-1-yl)-5-methyl-1H-pyrazol-1-yl)-2-azaspiro[3.3]heptane-2-carboxylate. The isomers were separated by chiral SFC (C-SFC-7; mobile phase: CO₂/MeOH: 85/15) to give tert-butyl (S)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate Intermediate C50a as the first eluting enantiomer; C-SFC-8 (mobile phase: CO₂/[MeOH+0.025% NH₃]: 85/15): Rt=2.72 min, UPLC-MS-4: Rt=1.07 min; MS m/z [M+H]⁺ 636.5 and tert-butyl (R)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate Intermediate C50b as the second eluting enantiomer: C-SFC-8 (mobile phase: CO₂/[MeOH+0.025% NH₃]: 85/15): Rt=3.12 min, UPLC-MS-4: Rt=1.07 min; MS m/z [M+H]⁺ 636.4.

### Method-C51 for the preparation of Intermediate C51: Tert-butyl (S)-6-(3-(2-(2-methoxyethyl)-2-methylpiperazin-1-yl)-5-methyl-1H-pyrazol-1-yl)-2-azaspiro[3.3]heptane-2-carboxylate

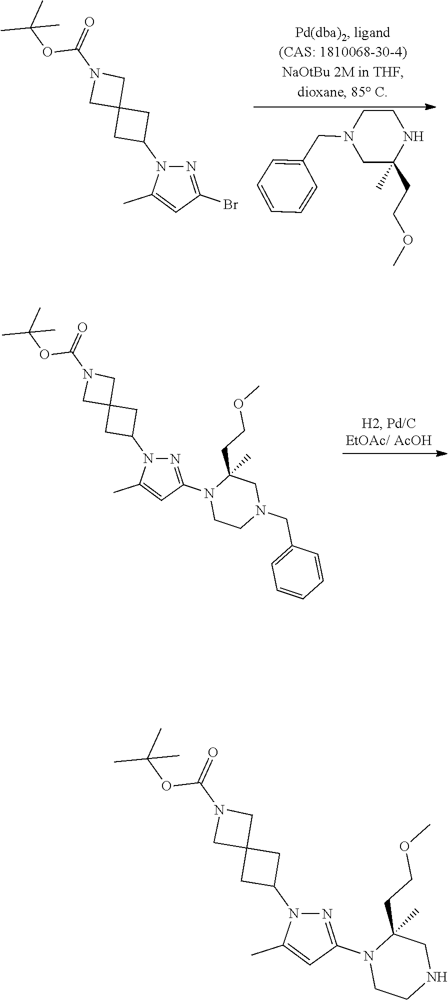

### Step 1: Tert-butyl (S)-6-(3-(4-benzyl-2-(2-methoxyethyl)-2-methylpiperazin-1-yl)-5-methyl-1H-pyrazol-1-yl)-2-azaspiro[3.3]heptane-2-carboxylate

Under inert atmosphere, to a stirred solution of tert-butyl 6-(3-bromo-5-methyl-1H-pyrazol-1-yl)-2-azaspiro[3.3]heptane-2-carboxylate (Intermediate C1, 3.00 g, 8.42 mmol), (S)-1-benzyl-3-(2-methoxyethyl)-3-methylpiperazine (Intermediate A38, 2.20 g, 8.84 mmol), bis(3,5-bis(trifluoromethyl)phenyl)(2′,6′-bis(dimethylamino)-3,6-dimethoxybiphenyl-2-yl)phosphine (CAS [1810068-30-4], 0.33 g, 0.43 mmol) and Pd(dba)₂ (0.24 g, 0.42 mmol) in 1,4-dioxane (40 mL) was added NaOtBu (2M in THF, 5.89 mL, 11.8 mmol). The reaction mixture was placed in a preheated bath (85° C.) and stirred at 85° C. for 6 h. After cooling to RT, the RM was poured into a sat. aq. NaHCO₃ solution and extracted with EtOAc. The combined organic layers were washed with brine, dried (Na₂SO₄), filtered and concentrated. The crude residue was purified by normal phase chromatography (eluent: EtOAc in c-hexane 0 to 75%) to give the title compound as a brown oil. UPLC-MS-4: Rt=0.91 min; MS m/z [M+H]⁺ 524.5.

### Step 2: Tert-butyl (S)-6-(3-(2-(2-methoxyethyl)-2-methylpiperazin-1-yl)-5-methyl-1H-pyrazol-1-yl)-2-azaspiro[3.3]heptane-2-carboxylate

Tert-butyl (S)-6-(3-(4-benzyl-2-(2-methoxyethyl)-2-methylpiperazin-1-yl)-5-methyl-1H-pyrazol-1-yl)-2-azaspiro[3.3]heptane-2-carboxylate (Step 1, 3.47 g, 6.63 mmol) was dissolved in a mixture of EtOAc (60 mL) and AcOH (3.79 mL) and 10% Pd/C (0.71 g, 0.66 mmol) was added. The reaction mixture was placed under hydrogen atmosphere and stirred at RT for 16 h. The RM was filtered through a celite pad and the filtrate was poured into a sat. aq. NaHCO₃ solution, then extracted with EtOAc (2×). The combined organic layers were washed with a sat. aq. NaHCO₃ solution, dried (Na₂SO₄), filtered and concentrated under vacuum. The crude residue was purified by normal phase chromatography (eluent: MeOH in CH₂Cl₂ 0 to 10%) to give the title compound as white solid. UPLC-MS-4: Rt=0.69 min; MS m/z [M+H]⁺ 434.5.

The following intermediates C52 to C54 were prepared using analogous methods to Method-C51 from intermediates described in the intermediates synthesis section or commercially available (in Step 1, 2 or 3).

| Intermediate | Structure | Precursor | Characterizing data |
|---|---|---|---|
| C52 | 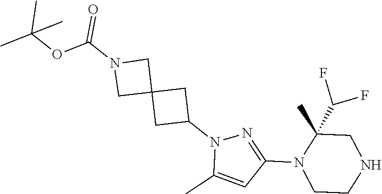 [SMILES: Cc1cc(N2CCNC[C@@]2(C)C(F)F)nn1C1CC2(C1)CN(C(=O)OC(C)(C)C)C2] | Using Method-C51 from Intermediate A35 (Step 1) | UPLC-MS-4: Rt = 0.74 min; MS m/z [M + H]⁺ 426.4. |
| | Tert-butyl (S)-6-(3-(2- | | |
| | (difluoromethyl)-2-methylpiperazin- | | |
| | 1-yl)-5-methyl-1H-pyrazol-1-yl)-2- | | |
| | azaspiro[3.3]heptane-2- | | |
| | carboxylate | | |
| C53 | 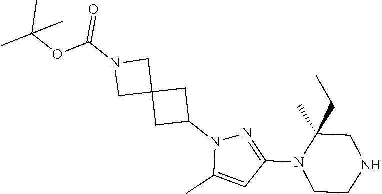 [SMILES: CC[C@@]1(C)CNCCN1c1cc(C)n(C2CC3(C2)CN(C(=O)OC(C)(C)C)C3)n1] | Using Method-C51 from Intermediate A32 (Step 1) | UPLC-MS-4: Rt = 0.67 min; MS m/z [M + H]⁺ 404.5. |
| | Tert-butyl (S)-6-(3-(2-ethyl-2- | | |
| | methylpiperazin-1-yl)-5-methyl-1H- | | |
| | pyrazol-1-yl)-2- | | |
| | azaspiro[3.3]heptane-2- | | |
| | carboxylate | | |
| C54 | 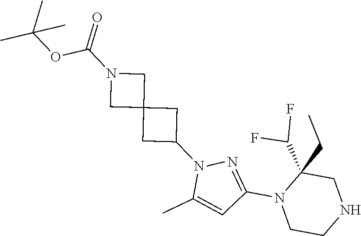 [SMILES: CC[C@@]1(C(F)F)CNCCN1c1cc(C)n(C2CC3(C2)CN(C(=O)OC(C)(C)C)C3)n1] | Using Method-C51 from Intermediate A36 (Step 1) | UPLC-MS: Rt = 0.71 min; MS m/z [M + H]⁺ 440.4. |
| | Tert-butyl (S)-6-(3-(2- | | |
| | (difluoromethyl)-2-ethylpiperazin-1- | | |
| | yl)-5-methyl-1H-pyrazol-1-yl)-2- | | |
| | azaspiro[3.3]heptane-2- | | |
| | carboxylate | | |

### Intermediate 055: Tert-butyl (S)-6-(3-(2-ethyl-2-methylpiperazin-1-yl)-4-iodo-5-methyl-1H-pyrazol-1-yl)-2-azaspiro[3.3]heptane-2-carboxylate

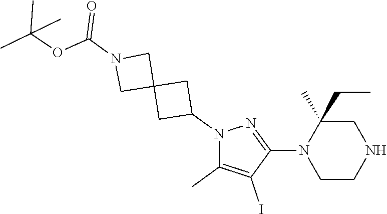 [SMILES: CC[C@@]1(C)CNCCN1c1nn(C2CC3(C2)CN(C(=O)OC(C)(C)C)C3)c(C)c1I]

To a solution of tert-butyl (S)-6-(3-(4-benzyl-2-ethyl-2-methylpiperazin-1-yl)-4-iodo-5-methyl-1H-pyrazol-1-yl)-2-azaspiro[3.3]heptane-2-carboxylate (prepared in Method-C39 (Step 2), 15.0 g, 22.0 mmol) in CH₂Cl₂ (150 mL) was added 1-chloroethyl chloroformate (4.75 mL, 44.1 mmol) and the reaction mixture was stirred for 1.5 h at 40° C. MeOH (50 mL) and a sat. aq. NaHCO₃ solution were added (100 mL) and the mixture was vigorously stirred for 30 min until disappearance of the carbamate intermediate. CH₂Cl₂ was added and the layers were separated, the organic layer was washed with a sat. aq. NaHCO₃, dried (phase separator) and concentrated. The crude residue was purified by normal phase chromatography (eluent: MeOH in CH₂Cl₂ 0 to 10%) to give the title product. UPLC-MS-4: Rt=0.90 min; MS m/z [M+H]⁺ 530.3.

### Intermediate C56: Tert-butyl (R)-6-(4-iodo-3-(2-(methoxymethyl)-2-methylpiperazin-1-yl)-5-methyl-1H-pyrazol-1-yl)-2-azaspiro[3.3]heptane-2-carboxylate

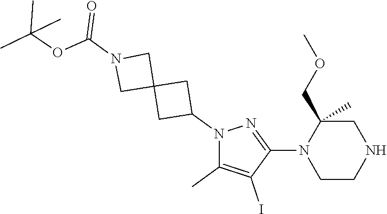 [SMILES: COC[C@@]1(C)CNCCN1c1nn(C2CC3(C2)CN(C(=O)OC(C)(C)C)C3)c(C)c1I]

The title compound was prepared by a method similar to Intermediate C55 starting from tert-butyl (R)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate (Intermediate C50b) instead of Tert-butyl (S)-6-(3-(4-benzyl-2-ethyl-2-methylpiperazin-1-yl)-4-iodo-5-methyl-1H-pyrazol-1-yl)-2-azaspiro[3.3]heptane-2-carboxylate. UPLC-MS-4: Rt=0.80 min; MS m/z [M+H]⁺ 546.3.

### Method-C57 for the preparation of Intermediate C57: Tert-butyl (S)-6-(3-(2-(2-methoxyethyl)-2-methyl-4-(oxetan-3-yl)piperazin-1-yl)-5-methyl-1H-pyrazol-1-yl)-2-azaspiro[3.3]heptane-2-carboxylate

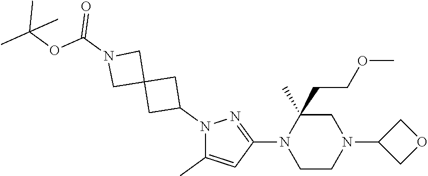 [SMILES: COCC[C@@]1(C)CN(C2COC2)CCN1c1cc(C)n(C2CC3(C2)CN(C(=O)OC(C)(C)C)C3)n1]

To a stirred solution of tert-butyl (S)-6-(3-(2-(2-methoxyethyl)-2-methylpiperazin-1-yl)-5-methyl-1H-pyrazol-1-yl)-2-azaspiro[3.3]heptane-2-carboxylate (intermediate C51, 1.08 g, 2.49 mmol) in CH₂Cl₂ (20 mL) were added under argon atmosphere 2-oxetanone (0.27 g, 3.74 mmol) and NaBH(OAc)₃ (1.58 g, 7.47 mmol). The reaction mixture was stirred for 1 h at RT. The RM was quenched by addition of a sat. aq. NaHCO₃ solution and extracted CH₂Cl₂ (2×). The combined organic layers were washed with a sat. aq. NaHCO₃ solution, dried (Na₂SO₄), filtered and evaporated. The crude residue was purified by normal phase chromatography (eluent: EtOAc in c-hexane from 0 to 100%). UPLC-MS-4: Rt=0.87 min; MS m/z [M+H]⁺ 490.5.

### Intermediate C58: Tert-butyl (S)-6-(3-(2-(2-methoxyethyl)-2-methyl-4-(oxetan-3-yl)piperazin-1-yl)-5-methyl-1H-pyrazol-1-yl)-2-azaspiro[3.3]heptane-2-carboxylate

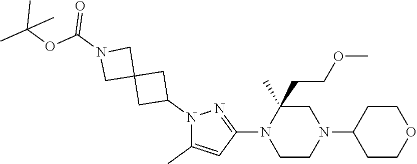 [SMILES: COCC[C@@]1(C)CN(C2CCOCC2)CCN1c1cc(C)n(C2CC3(C2)CN(C(=O)OC(C)(C)C)C3)n1]

The title compound was prepared by a method similar to Method-C57 using tetrahydro-4H-pyran-4-one instead of 2-oxetanone. UPLC-MS-4: Rt=0.73 min; MS m/z [M+H]⁺ 518.5.

### Intermediate C59: Tert-butyl (R)-6-(4-iodo-3-(2-(methoxymethyl)-2-methyl-4-(tetrahydro-2H-pyran-4-yl)piperazin-1-yl)-5-methyl-1H-pyrazol-1-yl)-2-azaspiro[3.3]heptane-2-carboxylate

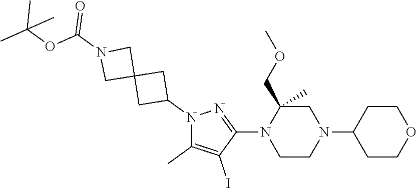 [SMILES: COC[C@@]1(C)CN(C2CCOCC2)CCN1c1nn(C2CC3(C2)CN(C(=O)OC(C)(C)C)C3)c(C)c1I]

The title compound was prepared by a method similar to Method-C57 starting from tert-butyl (R)-6-(4-iodo-3-(2-(methoxymethyl)-2-methylpiperazin-1-yl)-5-methyl-1H-pyrazol-1-yl)-2-azaspiro[3.3]heptane-2-carboxylate (Intermediate C56) and using tetrahydro-4H-pyran-4-one instead of 2-oxetanone. UPLC-MS-4: Rt=0.81 min; MS m/z [M+H]⁺ 630.5.

### Intermediate C60: Tert-butyl (S)-6-(3-(2-(difluoromethyl)-2-methyl-4-(tetrahydro-2H-pyran-4-yl)piperazin-1-yl)-5-methyl-1H-pyrazol-1-yl)-2-azaspiro[3.3]heptane-2-carboxylate

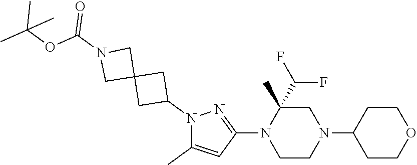 [SMILES: Cc1cc(N2CCN(C3CCOCC3)C[C@@]2(C)C(F)F)nn1C1CC2(C1)CN(C(=O)OC(C)(C)C)C2]

The title compound was prepared by a method similar to Method-C57 starting from tert-butyl (S)-6-(3-(2-(difluoromethyl)-2-methylpiperazin-1-yl)-5-methyl-1H-pyrazol-1-yl)-2-azaspiro[3.3]heptane-2-carboxylate (Intermediate C52) and using tetrahydro-4H-pyran-4-one instead of 2-oxetanone. UPLC-MS-4: Rt=0.87 min; MS m/z [M+H]⁺ 510.4.

### Intermediate C61: Tert-butyl (S)-6-(5-methyl-3-(2,2,5-trimethyl-4-(oxetan-3-yl)piperazin-1-yl)-1H-pyrazol-1-yl)-2-azaspiro[3.3]heptane-2-carboxylate

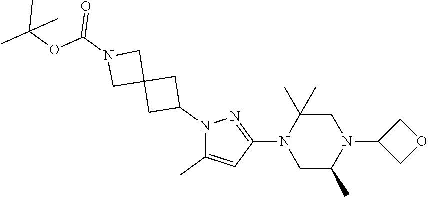 [SMILES: Cc1cc(N2C[C@H](C)N(C3COC3)CC2(C)C)nn1C1CC2(C1)CN(C(=O)OC(C)(C)C)C2]

The title compound was prepared by a method similar to Method-C57 starting from tert-butyl (S)-6-(5-methyl-3-(2,2,5-trimethylpiperazin-1-yl)-1H-pyrazol-1-yl)-2-azaspiro[3.3]heptane-2-carboxylate instead of intermediate C51. UPLC-MS-4: Rt=0.89 min; MS m/z [M+H]⁺ 460.4.

### Tert-butyl (S)-6-(5-methyl-3-(2,2,5-trimethylpiperazin-1-yl)-1H-pyrazol-1-yl)-2-azaspiro[3.3]heptane-2-carboxylate

The title compound was prepared by a method similar to Method-C51 starting from (S)-1-benzyl-2,5,5-trimethylpiperazine (Intermediate A39) instead Intermediate A38. UPLC-MS-4: Rt=0.73 min; MS m/z [M+H]⁺ 405.4.

### Intermediate C62: Tert-butyl 6-(3-((S)-4-(((R)-1,4-dioxan-2-yl)methyl)-2-(2-methoxyethyl)-2-methylpiperazin-1-yl)-5-methyl-1H-pyrazol-1-yl)-2-azaspiro[3.3]heptane-2-carboxylate

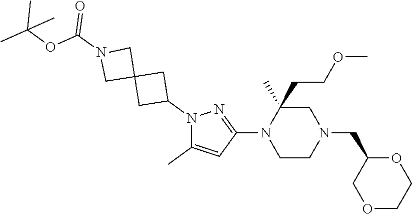 [SMILES: COCC[C@@]1(C)CN(C[C@@H]2COCCO2)CCN1c1cc(C)n(C2CC3(C2)CN(C(=O)OC(C)(C)C)C3)n1]

To a stirred solution of tert-butyl (S)-6-(3-(2-(2-methoxyethyl)-2-methylpiperazin-1-yl)-5-methyl-1H-pyrazol-1-yl)-2-azaspiro[3.3]heptane-2-carboxylate (intermediate C51, 1.18 g, 2.64 mmol) and (S)-(1,4-dioxan-2-yl)methyl 4-methylbenzenesulfonate (Intermediate B1, 1.09 g, 3.96 mmol) in CH₃CN (20 mL) were added under nitrogen atmosphere triethylamine (1.10 mL, 7.92 mmol) and sodium iodide (396 mg, 2.64 mmol) and the reaction mixture was stirred for 48 h at 80° C. The RM was poured into a sat. aq. NaHCO₃ solution, extracted with EtOAc (×3), the combined organic extracts were washed with brine, dried (Na₂SO₄), filtered and concentrated. The crude residue was purified by normal phase chromatography (eluent: EtOAc in n-heptane 0 to 100%) to give the title product. UPLC-MS-4: Rt=0.75 min; MS m/z [M+H]⁺ 534.5.

### Intermediate C63: Tert-butyl 6-(3-((S)-4-(((R)-1,4-dioxan-2-yl)methyl)-2-(difluoromethyl)-2-methylpiperazin-1-yl)-5-methyl-1H-pyrazol-1-yl)-2-azaspiro[3.3]heptane-2-carboxylate

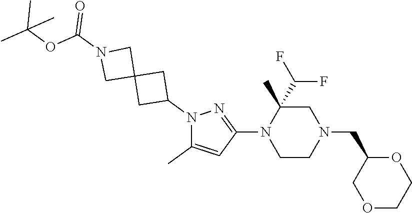 [SMILES: Cc1cc(N2CCN(C[C@@H]3COCCO3)C[C@@]2(C)C(F)F)nn1C1CC2(C1)CN(C(=O)OC(C)(C)C)C2]

The title compound was prepared by a method similar to Intermediate C62 starting from tert-butyl (S)-6-(3-(2-(difluoromethyl)-2-methylpiperazin-1-yl)-5-methyl-1H-pyrazol-1-yl)-2-azaspiro[3.3]heptane-2-carboxylate (Intermediate C52) instead of Intermediate C51. UPLC-MS-4: Rt=1.00 min; MS m/z [M+H]⁺ 526.5.

### Intermediate C64: Tert-butyl 6-(3-((S)-4-(((R)-1,4-dioxan-2-yl)methyl)-2-(difluoromethyl)-2-ethylpiperazin-1-yl)-5-methyl-1H-pyrazol-1-yl)-2-azaspiro[3.3]heptane-2-carboxylate

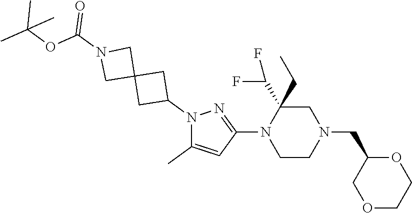 [SMILES: CC[C@@]1(C(F)F)CN(C[C@@H]2COCCO2)CCN1c1cc(C)n(C2CC3(C2)CN(C(=O)OC(C)(C)C)C3)n1]

The title compound was prepared by a method similar to Intermediate C62 starting from tert-butyl (S)-6-(3-(2-(difluoromethyl)-2-ethylpiperazin-1-yl)-5-methyl-1H-pyrazol-1-yl)-2-azaspiro[3.3]heptane-2-carboxylate (intermediate C54) instead of Intermediate C51. UPLC-MS-4: Rt=1.09 min; MS m/z [M+H]⁺ 540.4.

### Intermediate C65: Tert-butyl 6-(3-((S)-4-(((R)-1,4-dioxan-2-yl)methyl)-2-ethyl-2-methylpiperazin-1-yl)-4-iodo-5-methyl-1H-pyrazol-1-yl)-2-azaspiro[3.3]heptane-2-carboxylate

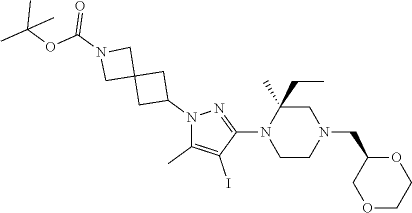 [SMILES: CC[C@@]1(C)CN(C[C@@H]2COCCO2)CCN1c1nn(C2CC3(C2)CN(C(=O)OC(C)(C)C)C3)c(C)c1I]

To a solution of tert-butyl (S)-6-(3-(2-ethyl-2-methylpiperazin-1-yl)-4-iodo-5-methyl-1H-pyrazol-1-yl)-2-azaspiro[3.3]heptane-2-carboxylate (Intermediate C55, 10.6 g, 17.2 mmol) and triethylamine (12.0 mL, 86 mmol) in CH₃CN (120 mL) was added 8 (S)-(1,4-dioxan-2-yl)methyl 4-methylbenzenesulfonate (Intermediate B1, 5.63 g, 20.7 mmol) and the reaction mixture was stirred for 7 d at 80° C. in an Ace-tube under nitrogen atmosphere. The reaction mixture was poured into a sat. aq. NaHCO₃ solution, extracted with EtOAc (×3), the combined organic extracts were washed with brine, dried (phase peparator) and concentrated. The crude residue was purified by normal phase chromatography (eluent: EtOAc in c-hexane 0 to 40%) to give the title product. UPLC-MS-4: Rt=0.96 min; MS m/z [M+H]⁺ 630.3.

### Intermediate C66: Tert-butyl 6-(3-((R)-4-(((R)-1,4-dioxan-2-yl)methyl)-2-(methoxymethyl)-2-methylpiperazin-1-yl)-4-iodo-5-methyl-1H-pyrazol-1-yl)-2-azaspiro[3.3]heptane-2-carboxylate

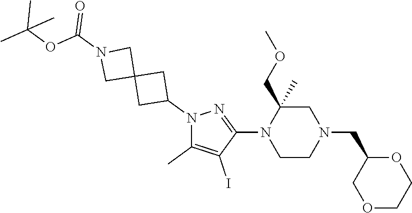 [SMILES: COC[C@@]1(C)CN(C[C@@H]2COCCO2)CCN1c1nn(C2CC3(C2)CN(C(=O)OC(C)(C)C)C3)c(C)c1I]

The title compound was prepared by a method similar to Intermediate C65 starting from tert-butyl (R)-6-(4-iodo-3-(2-(methoxymethyl)-2-methylpiperazin-1-yl)-5-methyl-1H-pyrazol-1-yl)-2-azaspiro[3.3]heptane-2-carboxylate (Intermediate C56). UPLC-MS-4: Rt=0.90 min; MS m/z [M+H]⁺ 646.4.

### Intermediate C67: Tert-butyl (S)-6-(3-(2-ethyl-2-methyl-4-((2-(trimethylsilyl)ethoxy)carbonyl)piperazin-1-yl)-5-methyl-1H-pyrazol-1-yl)-2-azaspiro[3.3]heptane-2-carboxylate

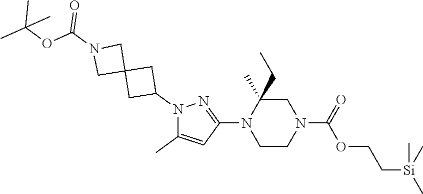 [SMILES: CC[C@@]1(C)CN(C(=O)OCC[Si](C)(C)C)CCN1c1cc(C)n(C2CC3(C2)CN(C(=O)OC(C)(C)C)C3)n1]

To an ice-cooled solution of tert-butyl (S)-6-(3-(2-ethyl-2-methylpiperazin-1-yl)-5-methyl-1H-pyrazol-1-yl)-2-azaspiro[3.3]heptane-2-carboxylate (Intermediate C53, 7.51 g, 18.6 mmol) in CH₂Cl₂ (93 mL) under inert atmosphere were added DIPEA (9.75 mL, 55.8 mmol) and 2,5-dioxopyrrolidin-1-yl (2-(trimethylsilyl)ethyl) carbonate (4.83 g, 18.6 mmol). The reaction mixture was stirred at RT for 1 h. The RM was quenched with a sat. aq. NaHCO₃ solution and extracted with CH₂Cl₂. The combined organic layer was washed with brine, then dried (MgSO₄), filtered and evaporated. The crude residue was purified by normal phase chromatography (eluent: TBME in n-heptane 0 to 100%) to give the title compound as a white solid. UPLC-MS-4: Rt=1.62 min; MS m/z [M+H]⁺ 548.5.

### Intermediate C68: Tert-butyl 6-(4-(5,6-dichloro-1-(tetrahydro-2H-pyran-2-yl)-1H-indazol-4-yl)-3-((R)-2-(methoxymethyl)-2-methylpiperazin-1-yl)-5-methyl-1H-pyrazol-1-yl)-2-azaspiro[3.3]heptane-2-carboxylate

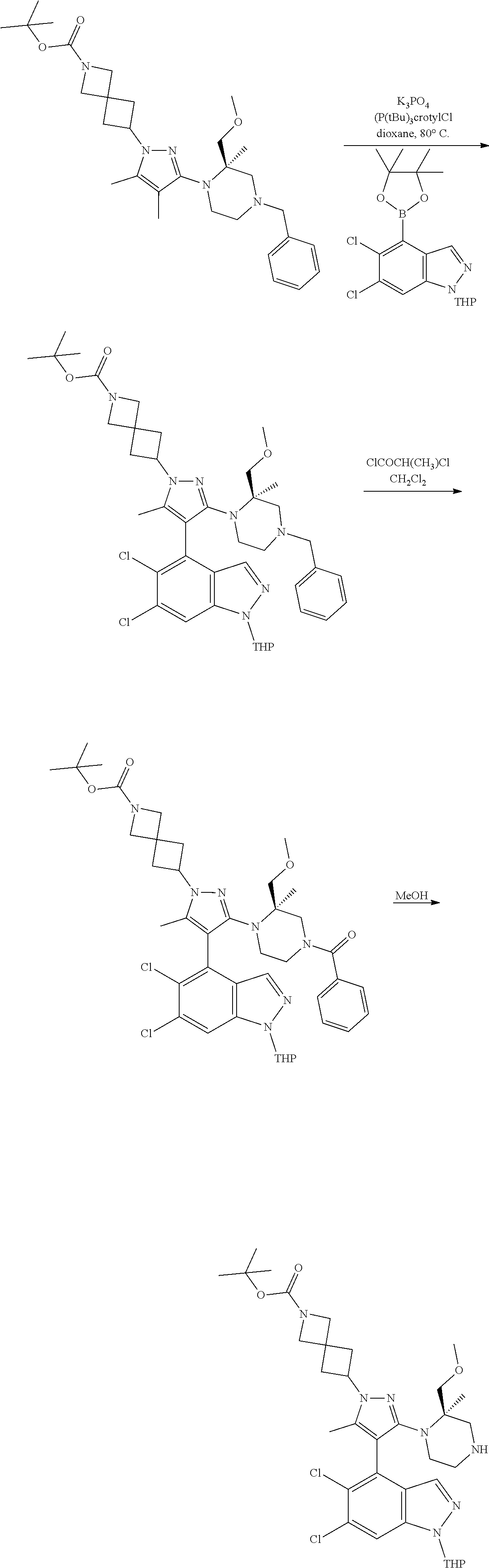

### Step 1: Tert-butyl 6-(3-((R)-4-benzyl-2-(methoxymethyl)-2-methylpiperazin-1-yl)-4-(5,6-dichloro-1-(tetrahydro-2H-pyran-2-yl)-1H-indazol-4-yl)-5-methyl-1H-pyrazol-1-yl)-2-azaspiro[3.3]heptane-2-carboxylate

To a solution of tert-butyl (R)-6-(3-(4-benzyl-2-(methoxymethyl)-2-methylpiperazin-1-yl)-4-iodo-5-methyl-1H-pyrazol-1-yl)-2-azaspiro[3.3]heptane-2-carboxylate (Intermediate C50b, 1.78 g, 2.80 mmol), 5,6-dichloro-1-(tetrahydro-2H-pyran-2-yl)-4-(4,4,5,5-tetramethyl-1,3,2-dioxaborolan-2-yl)-1H-indazole (Intermediate D6, 1.33 g, 3.36 mmol) and [P(tBu)₃] Pd(crotyl)Cl (0.11 g, 0.28 mmol) in dioxane (25 mL) was added under Argon atmosphere K₃PO₄ (1M in water, 3.64 mL, 3.64 mmol) and the reaction mixture was stirred at 80° C. for 18 h. The RM was poured into a sat. aq. NaHCO₃ solution and extracted with EtOAc (×2). The combined organic extracts were washed with a sat. aq. NaHCO₃, dried (Na₂SO₄), filtered and concentrated. The residue was swirled in CH₂Cl₂ with Si-TMT (Cas [1226494-16-1], 0.50 g, loading 0.5 mmol/g) for 1 h at 40° C., concentrated and purified by normal phase chromatography (eluent: EtOAc in c-hexane 0 to 48%) to give the title compound as a white solid. UPLC-MS-4: Rt=1.30 min; MS m/z [M+H]⁺ 778.4/780.4/782.4.

### Step 2: Tert-butyl 6-(3-((2R)-4-(2-chloropropanoyl)-2-(methoxymethyl)-2-methylpiperazin-1-yl)-4-(5,6-dichloro-1-(tetrahydro-2H-pyran-2-yl)-1H-indazol-4-yl)-5-methyl-1H-pyrazol-1-yl)-2-azaspiro[3.3]heptane-2-carboxylate

To a solution of tert-butyl 6-(3-((R)-4-benzyl-2-(methoxymethyl)-2-methylpiperazin-1-yl)-4-(5,6-dichloro-1-(tetrahydro-2H-pyran-2-yl)-1H-indazol-4-yl)-5-methyl-1H-pyrazol-1-yl)-2-azaspiro[3.3]heptane-2-carboxylate (Step 1, 1.59 g, 2.04 mmol) in CH₂Cl₂ (20 mL) was added 1-chloroethyl chloroformate (0.45 mL, 4.08 mmol) and the reaction mixture was stirred for 1 h at RT. The RM was poured into a sat. aq. NaHCO₃ solution and extracted with CH₂Cl₂. The combined organic extracts were washed with a sat. aq. NaHCO₃, dried (Na₂SO₄), filtered and concentrated. The crude residue was purified by normal phase chromatography (eluent: AcOEt in c-hexane 0 to 60%) to give the title product. UPLC-MS-4: Rt=1.13 min; MS m/z [M−COCH(CH₃)Cl]⁺ 688.5/690.5/692.5 .

### Step 3: Tert-butyl 6-(4-(5,6-dichloro-1-(tetrahydro-2H-pyran-2-yl)-1H-indazol-4-yl)-3-((R)-2-(methoxymethyl)-2-methylpiperazin-1-yl)-5-methyl-1H-pyrazol-1-yl)-2-azaspiro[3.3]heptane-2-carboxylate

A solution of tert-butyl 6-(3-((2R)-4-(2-chloropropanoyl)-2-(methoxymethyl)-2-methylpiperazin-1-yl)-4-(5,6-dichloro-1-(tetrahydro-2H-pyran-2-yl)-1H-indazol-4-yl)-5-methyl-1H-pyrazol-1-yl)-2-azaspiro[3.3]heptane-2-carboxylate (Step 2, 1.11 g, 1.42 mmol) in MeOH (20 mL) was stirred for 16 h at RT. Then the RM was evaporated and the crude residue was purified by normal phase chromatography (eluent: MeOH in CH₂Cl₂ 0 to 10%) to give the title product to give the title compound. Rt=1.13 min; MS m/z [M+H]⁺ 688.6/690.6/692.6.

### Intermediate C69: Tert-butyl 6-(4-(5-chloro-6-methyl-1-(tetrahydro-2H-pyran-2-yl)-1H-indazol-4-yl)-5-cyano-3-((S)-2-ethyl-2-methylpiperazin-1-yl)-1H-pyrazol-1-yl)-2-azaspiro[3.3]heptane-2-carboxylate

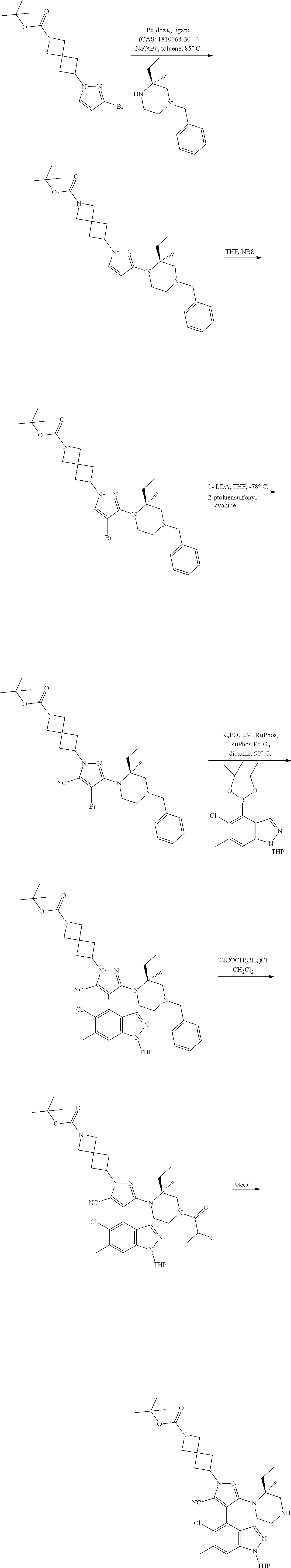

### Step 1: Tert-butyl (S)-6-(3-(4-benzyl-2-ethyl-2-methylpiperazin-1-yl)-1H-pyrazol-1-yl)-2-azaspiro[3.3]heptane-2-carboxylate

To a stirred solution of tert-butyl 6-(3-bromo-1H-pyrazol-1-yl)-2-azaspiro[3.3]heptane-2-carboxylate (Intermediate C4, 10.0 g, 29.2 mmol), (S)-1-benzyl-3-ethyl-3-methylpiperazine (Intermediate A32, 7.66 g, 35.1 mmol), bis(3,5-bis(trifluoromethyl)phenyl)(2′,6′-bis(dimethylamino)-3,6-dimethoxybiphenyl-2-yl)phosphine (CAS [1810068-30-4], 1.66 g, 2.19 mmol) and Pd(dba)₂ (1.26 g, 2.19 mmol) in toluene (250 mL) was added under Argon NaOtBu (2 M in THF, 21.9 mL, 43.8 mmol) and the reaction mixture was stirred for 16 h at 85° C. The RM was quenched with a sat. aq. NaHCO₃ solution and extracted with EtOAc (×2). The combined organic layer was washed with a sat. aq. NaHCO₃ solution, dried (Na₂SO₄), filtered and evaporated. The crude residue was purified by normal phase chromatography (eluent: EtOAc in c-hexane from 0% to 45%) to give the title compound as a brown oil. UPLC-MS-4: Rt=0.99 min; MS m/z [M+H]⁺ 480.4.

### Step 2: Tert-butyl (S)-6-(3-(4-benzyl-2-ethyl-2-methylpiperazin-1-yl)-4-bromo-1H-pyrazol-1-yl)-2-azaspiro[3.3]heptane-2-carboxylate

To a stirred solution of tert-butyl (S)-6-(3-(4-benzyl-2-ethyl-2-methylpiperazin-1-yl)-1H-pyrazol-1-yl)-2-azaspiro[3.3]heptane-2-carboxylate (Step 1, 4.79 g, 9.99 mmol) in THF (100 mL) under Ar was added NBS (1.95 g, 11.0 mmol) at RT and the reaction mixture was stirred for 30 min. The RM was quenched by addition of a sat. aq. NaHCO₃ solution and extracted with EtOAc (×2). The combined organic layer was washed with a sat. aq. NaHCO₃ solution, dried (Na₂SO₄), filtered and evaporated. The crude residue was purified by normal phase chromatography (eluent: EtOAc in c-hexane from 0 to 34%) to afford the title compound as a white solid. UPLC-MS-4: Rt=1.11 min; MS m/z [M+H]⁺ 558.4/560.4.

### Step 3: Tert-butyl (S)-6-(3-(4-benzyl-2-ethyl-2-methylpiperazin-1-yl)-4-bromo-5-cyano-1H-pyrazol-1-yl)-2-azaspiro[3.3]heptane-2-carboxylate

To a stirred solution of tert-butyl (S)-6-(3-(4-benzyl-2-ethyl-2-methylpiperazin-1-yl)-4-bromo-1H-pyrazol-1-yl)-2-azaspiro[3.3]heptane-2-carboxylate (Step 2, 1.66 g, 2.97 mmol) in THF (25 mL) under Ar was added LDA (1M in THF, 3.86 mL, 3.86 mmol) at −78° C. The mixture was stirred for 60 min at −78° C. and p-toluenesulfonyl cyanide (0.70 g, 3.86 mmol) was added. The reaction mixture was stirred for 30 min at −78° C. and was quenched with a sat. aq. NaHCO₃ solution. EtOAc was added, the layers were separated, and the aqueous layer was extracted with EtOAc (×2). The combined organic extracts were washed with a sat. aq. NaHCO₃ solution, dried (Na₂SO₄), filtered and evaporated. The crude residue was purified by normal phase chromatography (eluent: EtOAc in c-hexane from 0 to 35%) to afford the title compound as a white solid. UPLC-MS-4: Rt=1.36 min; MS m/z [M+H]⁺ 583.3/585.3.

### Step 4: Tert-butyl 6-(3-((S)-4-benzyl-2-ethyl-2-methylpiperazin-1-yl)-4-(5-chloro-6-methyl-1-(tetrahydro-2H-pyran-2-yl)-1H-indazol-4-yl)-5-cyano-1H-pyrazol-1-yl)-2-azaspiro[3.3]heptane-2-carboxylate

To a stirred solution of tert-butyl (S)-6-(3-(4-benzyl-2-ethyl-2-methylpiperazin-1-yl)-4-bromo-5-cyano-1H-pyrazol-1-yl)-2-azaspiro[3.3]heptane-2-carboxylate (Step 3, 1.46 g, 2.50 mmol), 5-chloro-6-methyl-1-(tetrahydro-2H-pyran-2-yl)-4-(4,4,5,5-tetramethyl-1,3,2-dioxaborolan-2-yl)-1H-indazole (Intermediate D1, 1.13 g, 3.00 mmol) and K₃PO₄ (1.59 g, 7.51 mmol) in dioxane (20 mL) and H₂O (4 mL) was added under Ar RuPhos (0.12 g, 0.25 mmol) and RuPhos-Pd-G3 (0.21 g, 0.25 mmol) and the reaction mixture was stirred for 1 h at 100° C. The RM was quenched with a sat. aq. NaHCO₃ solution and extracted with EtOAc (×2). The combined organic extracts were washed a sat. aq. NaHCO₃ solution, dried (Na₂SO₄), filtered and evaporated. The crude residue was purified by normal phase chromatography (eluent: EtOAc in c-hexane from 0 to 50%) to afford the title product as a white solid. UPLC-MS-4: Rt=1.45, 1.48 min; MS m/z [M+H]⁺ 753.8/755.8.

### Step 5: Tert-butyl 6-(4-(5-chloro-6-methyl-1-(tetrahydro-2H-pyran-2-yl)-1H-indazol-4-yl)-3-((2S)-4-(2-chloropropanoyl)-2-ethyl-2-methylpiperazin-1-yl)-5-cyano-1H-pyrazol-1-yl)-2-azaspiro[3.3]heptane-2-carboxylate

To a stirred solution of tert-butyl 6-(3-((S)-4-benzyl-2-ethyl-2-methylpiperazin-1-yl)-4-(5-chloro-6-methyl-1-(tetrahydro-2H-pyran-2-yl)-1H-indazol-4-yl)-5-cyano-1H-pyrazol-1-yl)-2-azaspiro[3.3]heptane-2-carboxylate (Step 4, 1.96 g, 2.60 mmol) in CH₂Cl₂ (40 mL) was added under Argon 1-chloroethyl chloroformate (0.57 mL, 5.20 mmol) and the reaction mixture was stirred for 16 h at RT. The RM was quenched with a sat. aq. NaHCO₃ solution, extracted with CH₂Cl₂ (×2) and the combined organic layers were washed with a sat. aq. NaHCO₃ solution, dried (Na₂SO₄), filtered and evaporated. The crude residue was purified by normal phase chromatography (eluent: EtOAc in c-hexane from 0 to 50%) to afford the title compound as a white solid. UPLC-MS-4: Rt=1.03 min; MS m/z [M−COCH(Cl)CH₃+H]⁺663.6/665.6.

### Step 6: Tert-butyl 6-(4-(5-chloro-6-methyl-1-(tetrahydro-2H-pyran-2-yl)-1H-indazol-4-yl)-5-cyano-3-((S)-2-ethyl-2-methylpiperazin-1-yl)-1H-pyrazol-1-yl)-2-azaspiro[3.3]heptane-2-carboxylate

Tert-butyl 6-(4-(5-chloro-6-methyl-1-(tetrahydro-2H-pyran-2-yl)-1H-indazol-4-yl)-3-((2S)-4-(2-chloropropanoyl)-2-ethyl-2-methylpiperazin-1-yl)-5-cyano-1H-pyrazol-1-yl)-2-azaspiro[3.3]heptane-2-carboxylate (Step 5, 1.11 g, 1.47 mmol) was stirred in MeOH (10 mL) for 16 h at RT. After completion of the reaction, the RM was concentrated and the crude residue was purified by normal phase chromatography (eluent: (MeOH/NH₄OH:80/20) in CH₂Cl₂ from 0 to 10%) to give the title product as a white solid. UPLC-MS-4: Rt=1.02 min; MS m/z [M−COCH(Cl)CH₃+H]⁺663.6/665.6.

### Intermediate C70: Tert-butyl (R)-6-(4-bromo-3-(2,2-dimethyl-4-(morpholinomethyl)piperidin-1-yl)-1H-pyrazol-1-yl)-2-azaspiro[3.3]heptane-2-carboxylate

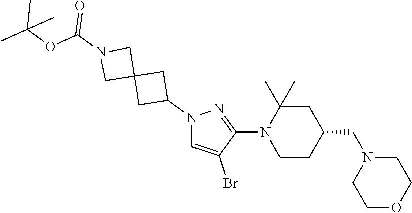 [SMILES: CC(C)(C)OC(=O)N1CC2(CC(n3cc(Br)c(N4CC[C@@H](CN5CCOCC5)CC4(C)C)n3)C2)C1]

The title compound was prepared by a method similar to Intermediate C69 (Step 1 and 2) starting from (R)-4-((2,2-dimethylpiperidin-4-yl)methyl)morpholine tert-butyl (Intermediate A70) intead of (S)-1-benzyl-3-ethyl-3-methylpiperazine (Intermediate A32) in Step 1. UPLC-MS-4: Rt=0.84 min; MS m/z [M+H]⁺ 552.4/554.4.

### Intermediate C71: Tert-butyl (R)-6-(4-bromo-3-(2,2-dimethyl-4-(morpholinomethyl)piperidin-1-yl)-5-(methyl-d₃)-1H-pyrazol-1-yl)-2-azaspiro[3.3]heptane-2-carboxylate

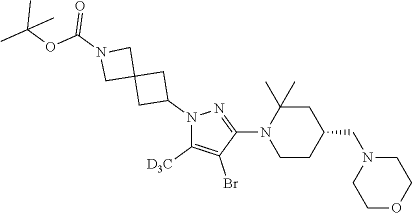 [SMILES: [2H]C([2H])([2H])c1c(Br)c(N2CC[C@@H](CN3CCOCC3)CC2(C)C)nn1C1CC2(C1)CN(C(=O)OC(C)(C)C)C2]

To a stirred solution of tert-butyl (R)-6-(4-bromo-3-(2,2-dimethyl-4-(morpholinomethyl)piperidin-1-yl)-1H-pyrazol-1-yl)-2-azaspiro[3.3]heptane-2-carboxylate (Intermediate C70, 1.40 g, 2.53 mmol) in THF (25 mL) under Ar was added LDA (1M in THF, 3.80 mL, 3.80 mmol) at −78° C. The reaction mixture was stirred for 60 min at −78° C. and iodomethane-d₃ (0.24 mL, 3.80 mmol) was added. The RM was stirred for 30 min at −78° C., then allowed to reach RT and further stirred for 2 h. The RM was quenched with a sat. aq. NaHCO₃ solution. EtOAc was added, the layers were separated, and the aqueous layer was extracted with EtOAc (×2). The combined organic extracts were washed with a sat. aq. NaHCO₃ solution, dried (Na₂SO₄), filtered and evaporated. The crude residue was purified by normal phase chromatography (eluent: EtOAc in c-hexane from 0 to 50%) to afford the title compound as a white solid. UPLC-MS-4: Rt=0.96 min; MS m/z [M+H]⁺ 569.8/571.8.

### Intermediate C72: Tert-butyl (R)-6-(4-bromo-5-cyano-3-(2,2-dimethyl-4-(morpholinomethyl)piperidin-1-yl)-1H-pyrazol-1-yl)-2-azaspiro[3.3]heptane-2-carboxylate

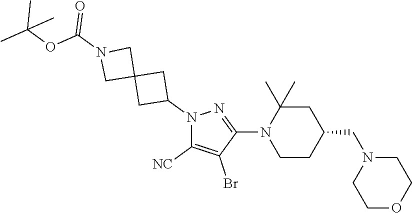 [SMILES: CC(C)(C)OC(=O)N1CC2(CC(n3nc(N4CC[C@@H](CN5CCOCC5)CC4(C)C)c(Br)c3C#N)C2)C1]

The title compound was prepared by a method similar to Intermediate C69 (Step 1, 2 and 3) starting from (R)-4-((2,2-dimethylpiperidin-4-yl)methyl)morpholine tert-butyl (Intermediate A70) instead of (S)-1-benzyl-3-ethyl-3-methylpiperazine (Intermediate A32) in Step 1. UPLC-MS-4: Rt=0.89 min; MS m/z [M+H]⁺ 577.2/579.2.

### Intermediate C73: Tert-butyl 6-(4-(2-chloro-5-(methoxymethoxy)-3,6-dimethylphenyl)-5-methyl-3-(8-(oxetan-3-yl)-5,8-diazaspiro[3.5]nonan-5-yl)-1H-pyrazol-1-yl)-2-azaspiro[3.3]heptane-2-carboxylate

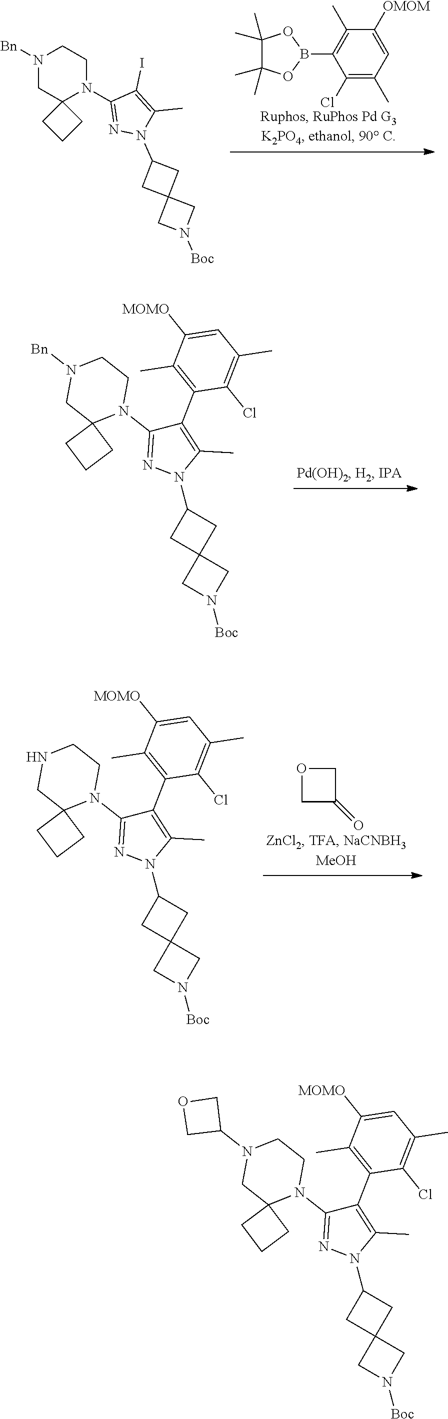

### Step 1: Tert-butyl 6-(3-(8-benzyl-5,8-diazaspiro[3.5]nonan-5-yl)-4-(2-chloro-5-(methoxymethoxy)-3,6-dimethylphenyl)-5-methyl-1H-pyrazol-1-yl)-2-azaspiro[3.3]heptane-2-carboxylate

Tert-butyl 6-(3-(8-benzyl-5,8-diazaspiro[3.5]nonan-5-yl)-4-iodo-5-methyl-1H-pyrazol-1-yl)-2-azaspiro[3.3]heptane-2-carboxylate (Prepared in Method-C28-B (Step 2), 0.50 g, 0.81 mmol), 2-(2-chloro-5-(methoxymethoxy)-3,6-dimethylphenyl)-4,4,5,5-tetramethyl-1,3,2-dioxaborolane (Intermediate D15, 0.53 g, 1.62 mmol) and K₃PO₄ (2M in water, 1.21 mL, 2.43 mmol) were added in EtOH (15 mL). The reaction mixture was degassed with nitrogen for 5 min. RuPhos (0.075 g, 0.16 mmol) and RuPhos-Pd-G3 (0.135 g, 0.16 mmol) were added and the RM was stirred at 90° C. for 30 min. After completion of the reaction, the RM was quenched with water and extracted with EtOAc (×2). The combined organic layer was washed with brine, dried (Na₂SO₄), filtered and concentrated under vacuum. The crude residue was purified by reverse phase chromatography (eluent: 0 to 48% CH₃CN in water containing 0.1% HCOOH) to obtain the title product. UPLC-MS-5: Rt=2.26 min, MS m/z [M+H]⁺ 690.4/692.3.

### Step 2: Tert-butyl 6-(4-(2-chloro-5-(methoxymethoxy)-3,6-dimethylphenyl)-5-methyl-3-(5,8-diazaspiro[3.5]nonan-5-yl)-1H-pyrazol-1-yl)-2-azaspiro[3.3]heptane-2-carboxyate

Tert-butyl 6-(3-(8-benzyl-5,8-diazaspiro[3.5]nonan-5-yl)-4-(2-chloro-5-(methoxymethoxy)-3,6-dimethylphenyl)-5-methyl-1H-pyrazol-1-yl)-2-azaspiro[3.3]heptane-2-carboxylate (Step 1, 0.36 g, 0.52 mmol) was dissolved in isopropanol (10 mL). Pd(OH)₂ (20%) (0.36 g, 0.31 mmol) was added and the reaction mixture was stirred at RT under hydrogen pressure (1 atmosphere) for 6 h. The RM was filtered through a pad of celite. The filtrate was concentrated under vacuum and the residue was purified by reverse phase combiflash (eluent: 0 to 60% CH₃CN in water containing in 0.025% NH₃) to give the title product. UPLC-MS-9: Rt=1.39 min, MS m/z [M+H]⁺ 600.6/602.5.

### Step 3: Tert-butyl 6-(4-(2-chloro-5-(methoxymethoxy)-3,6-dimethylphenyl)-5-methyl-3-(8-(oxetan-3-yl)-5,8-diazaspiro[3.5]nonan-5-yl)-1H-pyrazol-1-yl)-2-azaspiro[3.3]heptane-2-carboxyate

Tert-butyl 6-(4-(2-chloro-5-(methoxymethoxy)-3,6-dimethylphenyl)-5-methyl-3-(5,8-diazaspiro[3.5]nonan-5-yl)-1H-pyrazol-1-yl)-2-azaspiro[3.3]heptane-2-carboxylate (Step 2, 0.29 g, 0.48 mmol) and 3-oxetanone (0.07 g, 0.97 mmol) were dissolved in methanol (4 mL). The mixture was cooled to 0° C. under a nitrogen atmosphere and ZnCl₂ (0.19 g, 1.45 mmol) and TFA (0.11 g, 2.90 mmol) were added. The mixture was stirred for 10 min at 0° C. before the addition of NaBH₃CN (0.10 g, 1.50 mmol). The reaction mixture was allowed to reach RT and stirred at RT for 16 h. After completion of the reaction, the RM was diluted with water and extracted with EtOAc (×2). The combined organic layer was washed with brine, dried (Na₂SO₄), filtered and concentrated under vacuum to give the title compound which was used in the next step without further purification. UPLC-MS-9: Rt=1.42 min, MS m/z [M+H]⁺ 656.4/658.4.

### Intermediate C74: Tert-butyl 6-(3-(2,2-dimethyl-4-oxopiperidin-1-yl)-5-methyl-1H-pyrazol-1-yl)-2-azaspiro[3.3]heptane-2-carboxylate

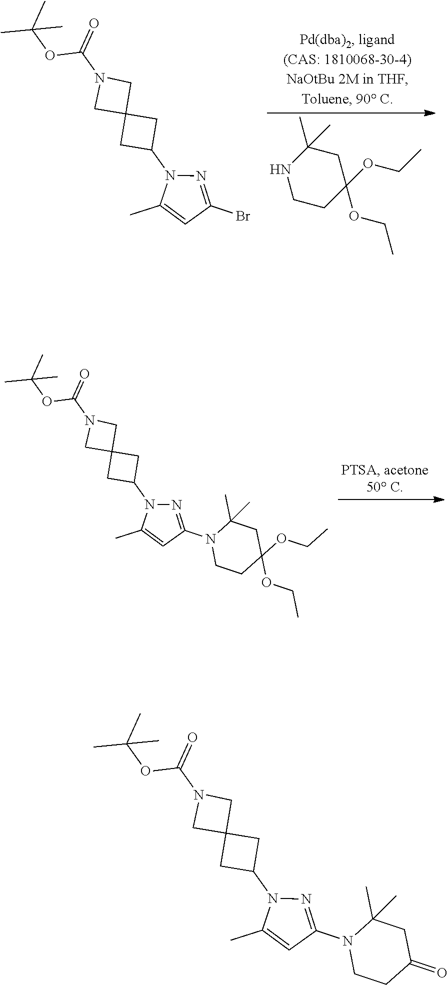

### Step 1: Tert-butyl 6-(3-(4,4-diethoxy-2,2-dimethylpiperidin-1-yl)-5-methyl-1H-pyrazol-1-yl)-2-azaspiro[3.3]heptane-2-carboxylate

A stirred solution of tert-butyl 6-(3-bromo-5-methyl-1H-pyrazol-1-yl)-2-azaspiro[3.3]heptane-2-carboxylate (Intermediate C1, 15.8 g, 44.3 mmol) and 4,4-diethoxy-2,2-dimethylpiperidine (Intermediate A47, 11.6 g, 57.7 mmol) in toluene (320 mL) was degassed with argon and Pd(dba)₂ (2.04 g, 3.55 mmol), bis(3,5-bis(trifluoromethyl)phenyl)(2′,6′-bis(dimethylamino)-3,6-dimethoxybiphenyl-2-yl)phosphine (CAS: 1810068-30-4, 2.68 g, 3.55 mmol) were added followed by the addition of NaOtBu (2M in THF, 66.5 mL, 133 mmol). The reaction mixture was placed in a preheated bath (90° C.) and stirred at 90° C. for 5 h. After cooling to RT, the RM was poured into a mixture of ice and water and extracted with EtOAc (×2). The combined organic layers were washed with brine, dried (Na₂SO₄), filtered and concentrated. The crude residue was purified by normal phase chromatography (eluent: [EtOAc+1% Et₃N] in n-heptane 0 to 40%) to give the title compound as brown oil. ¹H NMR (400 MHz, DMSO-d₆) δ 5.56 (s, 1H), 4.53 (m, 1H), 3.91 (m, 2H), 3.83 (m, 2H), 3.43-3.36 (m, 4H), 3.01 (m, 2H), 2.54 (m, 4H), 2.11 (s, 3H), 1.72 (m, 2H), 1.37 (s, 9H), 1.13 (s, 6H), 1.10 (t, 6H).

### Step 2: Tert-butyl 6-(3-(2,2-dimethyl-4-oxopiperidin-1-yl)-5-methyl-1H-pyrazol-1-yl)-2-azaspiro[3.3]heptane-2-carboxylate

A solution of tert-butyl 6-(3-(4,4-diethoxy-2,2-dimethylpiperidin-1-yl)-5-methyl-1H-pyrazol-1-yl)-2-azaspiro[3.3]heptane-2-carboxylate (9.54 g, 20.0 mmol) and PTSA (1.52 g, 8.01 mmol) in acetone (160 mL) was stirred overnight at 50° C. The reaction mixture was evaporated to ¼ of its volume and the residue was diluted with AcOEt. The organic layer was washed with a sat. aq. Na₂CO₃ solution and with brine. The organic layer was dried (Na₂SO₄), filtered and concentrated. The crude residue was purified by normal phase chromatography (eluent: EtOAc+1% Et₃N in heptane 0 to 100%) to give the title compound as a yellow oil. UPLC-MS-4: Rt=1.11 min; MS m/z [M+H]⁺ 403.4.

### Intermediate C75: Tert-butyl 6-(5-methyl-3-(8-oxo-5-azaspiro[3.5]nonan-5-yl)-1H-pyrazol-1-yl)-2-azaspiro[3.3]heptane-2-carboxylate

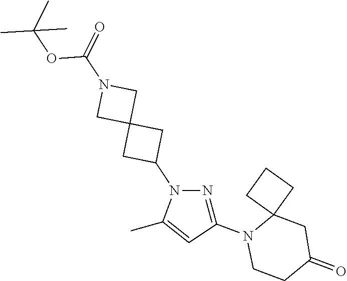 [SMILES: Cc1cc(N2CCC(=O)CC23CCC3)nn1C1CC2(C1)CN(C(=O)OC(C)(C)C)C2]

The title compound was prepared by a method similar to that of Intermediate C74 using 8,8-diethoxy-5-azaspiro[3.5]nonane (intermediate A48) instead of 4,4-diethoxy-2,2-dimethylpiperidine (intermediate A47) and 0.1 equivalent of PTSA instead of 0.4. UPLC-MS-4: Rt=1.24 min; MS m/z [M+H]⁺ 415.3.

### Intermediates C76a and C76b: Tert-butyl 6-(3-(4-hydroxy-2,2-dimethylpiperidin-1-yl)-5-methyl-1H-pyrazol-1-yl)-2-azaspiro[3.3]heptane-2-carboxylate

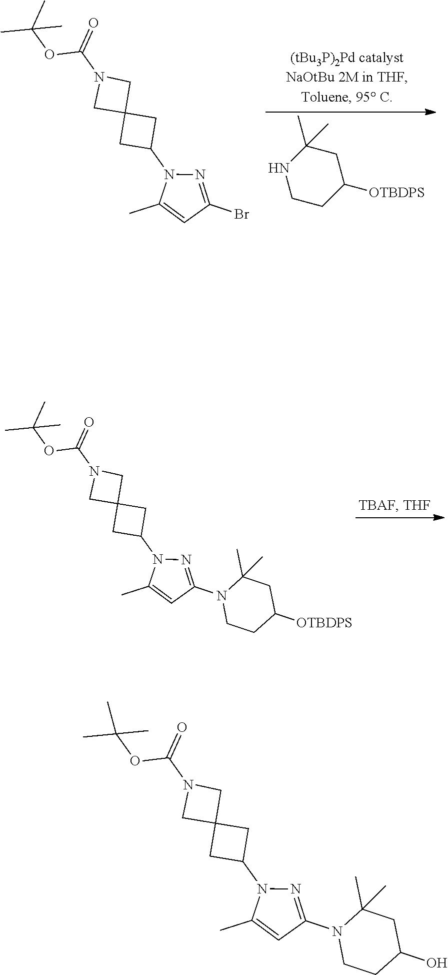

### Step 1: Tert-butyl 6-(3-(4-((tert-butyldiphenylsilyl)oxy)-2,2-dimethylpiperidin-1-yl)-5-methyl-1H-pyrazol-1-yl)-2-azaspiro[3.3]heptane-2-carboxylate

A solution of tert-butyl 6-(3-bromo-5-methyl-1H-pyrazol-1-yl)-2-azaspiro[3.3]heptane-2-carboxylate (Intermediate C1, 1.62 g, 4.56 mmol) and 4-((tert-butyldiphenylsilyl)oxy)-2,2-dimethylpiperidine (Intermediate A56, 1.40 g, 3.81 mmol) in toluene (20 mL) was purged with nitrogen gas for about 10 min. NaOtBu (2M in THF, 5.70 mL, 13.7 mmol) and bis(tri-tert-butylphosphine)palladium (CAS [53199-31-8], 0.19 g, 0.38 mmol) were added and the reaction mixture was stirred at 95° C. for 6 h in a sealed tube. After completion of the reaction, the RM was filtered through a pad of celite and the filtrate was concentrated under vacuum. The crude residue was purified by reverse phase chromatography (eluent: 0 to 60% CH₃CN in H₂O containing 0.1% NH₃) to give the title compound. LCMS-2: Rt=2.94 min; MS m/z [M+H]⁺ 643.7.

### Step 2: Tert-butyl 6-(3-(4-hydroxy-2,2-dimethylpiperidin-1-yl)-5-methyl-1H-pyrazol-1-yl)-2-azaspiro[3.3]heptane-2-carboxylate

To a solution of tert-butyl 6-(3-(4-((tert-butyldiphenylsilyl)oxy)-2,2-dimethylpiperidin-1-yl)-5-methyl-1H-pyrazol-1-yl)-2-azaspiro[3.3]heptane-2-carboxylate (Step 1, 1.10 g, 1.71 mmol) in dry THF (20 mL) under nitrogen atmosphere was added at 0° C. TBAF (1.0 M in THF, 5.13 mL, 5.13 mmol). The RM was slowly allowed to reach RT and stirred at RT for 14 h. The reaction mixture was diluted with water and extracted with EtOAc (×2). The combined organic extracts were dried (Na₂SO₄), filtered and concentrated under vacuum. The crude residue was purified by reverse phase chromatography (eluent: 0 to 100% CH₃CN in H₂O containing 0.1% NH₃) to give the title compound. The enantiomers were separated by chiral preparative SFC (C-SFC-47; mobile phase: CO₂/[CH₃CN/IPA 50/50+0.1% DEA]: 85/15) to give the first eluting enantiomer of the title compound: Intermediate C76a 1; C-SFC-48 (mobile phase: CO₂/[IPA/CH₃CN 50/50+0.1% DEA]: 95/5 to 50/50): Rt=4.67 min, UPLC-MS-5: Rt=1.45 min; MS m/z [M+H]⁺ 405.7 and the second eluting enantiomer of the title compound: Intermediate C76b: C-SFC-48 (mobile phase: CO₂/[IPA/CH₃CN 50/50+0.1% DEA]: 95/5 to 50/50): Rt=5.12 min, UPLC-MS-5: Rt=1.45 min; MS m/z [M+H]⁺ 405.7.

### Intermediate C77: Tert-butyl (R) and (S)-6-(3-(2,2-dimethyl-4-(2-morpholinoethoxy)piperidin-1-yl)-5-methyl-1H-pyrazol-1-yl)-2-azaspiro[3.3]heptane-2-carboxylate

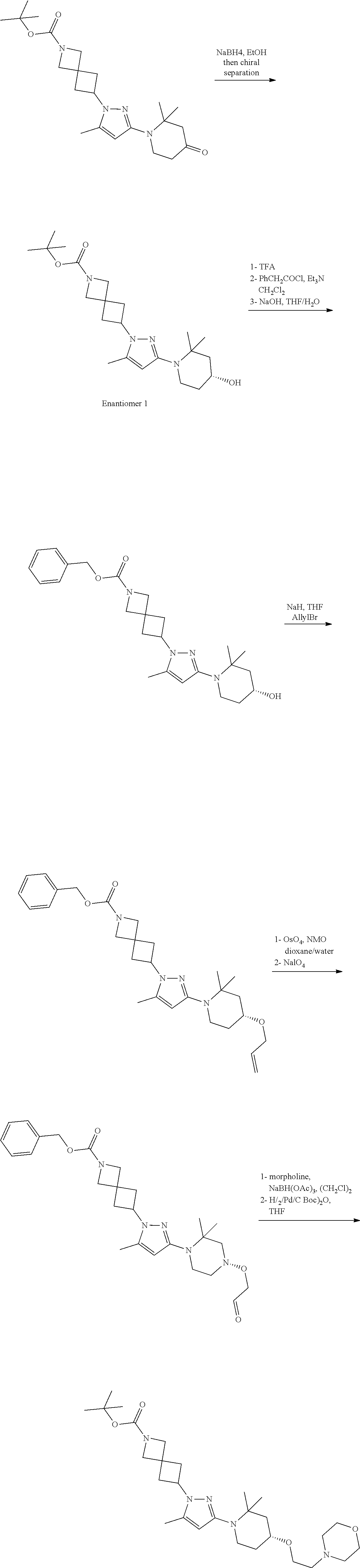

### Step 1: Tert-butyl (R) and (S)-6-(3-(4-hydroxy-2,2-dimethylpiperidin-1-yl)-5-methyl-1H-pyrazol-1-yl)-2-azaspiro[3.3]heptane-2-carboxylate

To a solution of tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 4.00 g, 9.95 mmol) in EtOH (40 mL) at 0° C. was added portion-wise added NaBH₄ (1.51 g, 39.8 mmol) and the reaction mixture was allowed to reach RT and was stirred at RT for 2 h. After completion of reaction, the RM was quenched by addition of a sat. aq. NaHCO₃ solution and extracted with EtOAc. The combined organic extracts were washed with brine, dried (Na₂SO₄), filtered and concentrated under vacuum. The crude residue was purified by reverse phase chormatography (eluent: 0 to 35% CH₃CN in H₂O (containg 0.1% HCO₂H)), to give the title compound as oil after basic work-up. The enantiomers were separated by chiral SFC C-SFC-47 (mobile phase: CO₂/[CH₃CN/IPA+0.1% Et₂NH (70/30)]: 85/15) to give the first eluting enantiomer of the title compound: Tert-butyl (R)-6-(3-(2,2-dimethyl-4-(2-morpholinoethoxy)piperidin-1-yl)-5-methyl-1H-pyrazol-1-yl)-2-azaspiro[3.3]heptane-2-carboxylate: C-SFC-31 (mobile phase: CO₂/[CH₃CN/IPA+0.1% Et₂NH (70/30)]: 85/15): Rt=3.95 min, UPLC-MS-4: Rt=0.73 min; MS m/z [M+H]⁺ 405.4 and the second eluting enantiomer of the title compound: Tert-butyl (S)-6-(3-(2,2-dimethyl-4-(2-morpholinoethoxy)piperidin-1-yl)-5-methyl-1H-pyrazol-1-yl)-2-azaspiro[3.3]heptane-2-carboxylate: C-SFC-31 (mobile phase: CO₂/[CH₃CN/IPA+0.1% Et₂NH (70/30)]: 85/15): Rt=6.40 min, UPLC-MS-4: Rt=0.73 min; MS m/z [M+H]⁺ 405.4.

### Step 2: (R)-2,2-dimethyl-1-(5-methyl-1-(2-azaspiro[3.3]heptan-6-yl)-1H-pyrazol-3-yl)piperidin-4-yl 2,2,2-trifluoroacetate

To a solution of tert-butyl (R)-6-(3-(4-hydroxy-2,2-dimethylpiperidin-1-yl)-5-methyl-1H-pyrazol-1-yl)-2-azaspiro[3.3]heptane-2-carboxylate (Step 1 first eluting enantiomer, 1.95 g, 4.82 mmol) in CH₂Cl₂ (25 mL) was added TFA (11.1 mL, 145 mmol) and the reaction mixture was stirred at RT for 16 h. Volatiles were removed in vacuo, and the residue was co-evaporated with CH₂Cl₂ (×2) to give the title compound as a trifluoroacetate salt (brown oil). UPLC-MS-4: Rt=0.79 min; MS m/z [M+H]⁺ 401.3.

### Step 3: Benzyl (R)-6-(3-(4-hydroxy-2,2-dimethylpiperidin-1-yl)-5-methyl-1H-pyrazol-1-yl)-2-azaspiro[3.3]heptane-2-carboxylate

To a solution of (R)-2,2-dimethyl-1-(5-methyl-1-(2-azaspiro[3.3]heptan-6-yl)-1H-pyrazol-3-yl)piperidin-4-yl 2,2,2-trifluoroacetate (Step 2, 4.78 g, 4.93 mmol) in CH₂Cl₂ (50 mL) was added at 0° C. triethylamine (7.55 mL, 54.2 mmol) followed by benzyl chloroformate (0.77 mL, 5.42 mmol). The reaction mixture was allowed to slowly reach RT and was stirred for 16 h at RT. The RM was poured into water and extracted CH₂Cl₂ (×2). The combined organic extracts were dried (phase separator) and evaporated. The crude residue was dissolved in THF (25 mL) and water (25 mL), NaOH (0.98 g, 24.6 mmol) was added and the RM was stirred at RT for 16 h. The reaction mixture was poured into water and extracted CH₂Cl₂ (×2). The combined organic extracts were dried (phase separator) and evaporated. The crude residue was purified by normal phase chromatography (eluent: MeOH in CH₂Cl₂ from 0 to 20%) to give the title compound as a white foam. UPLC-MS-4: Rt=0.76 min; MS m/z [M+H]⁺ 439.4.

### Step 4: Benzyl (R)-6-(3-(4-(allyloxy)-2,2-dimethylpiperidin-1-yl)-5-methyl-1H-pyrazol-1-yl)-2-azaspiro[3.3]heptane-2-carboxylate

To a solution of benzyl (R)-6-(3-(4-hydroxy-2,2-dimethylpiperidin-1-yl)-5-methyl-1H-pyrazol-1-yl)-2-azaspiro[3.3]heptane-2-carboxylate (Step 3, 1.10 g, 2.51 mmol) in THF (25 mL) was added at 0° C. NaH (0.30 g, 7.52 mmol) under a nitrogen atmsophere. The reaction mixture was stirred 1 h at 0° C. before addition of allyl bromide (0.32 mL, 3.76 mmol). The RM was slowly allowed to reach RT and was stirred for 16 h. Allyl bromide (0.32 mL, 3.76 mmol) was added again and the RM was further stirred at RT for 16 h. The RM was quenched by addition of ice and extracted with EtOAc (×2). The combined organic extracts were washed with brine, dried (Na₂SO₄), filtered and concentrated. The crude residue was purified by normal phase chromatography (eluent: EtOAc in n-heptane from 0 to 100%, then 20% MeOH in CH₂Cl₂) to give the title compound as a yellow oil. UPLC-MS-4: Rt=1.15 min; MS m/z [M+H]⁺ 479.6.

### Step 5: Benzyl (R)-6-(3-(2,2-dimethyl-4-(2-oxoethoxy)piperidin-1-yl)-5-methyl-1H-pyrazol-1-yl)-2-azaspiro[3.3]heptane-2-carboxylate

A solution of benzyl (R)-6-(3-(4-(allyloxy)-2,2-dimethylpiperidin-1-yl)-5-methyl-1H-pyrazol-1-yl)-2-azaspiro[3.3]heptane-2-carboxylate (Step 4, 1.15 g, 2.40 mmol), osmium tetroxide (2.5% in water, 6.03 mL, 0.48 mmol) and N-methylmorpholine oxide hydrate (0.36 g, 2.64 mmol) in dioxane (18.0 mL) and water (6.00 mL) was stirred 2 h at RT. After completion of the reaction, NaIO₄ (5.14 g, 24.0 mmol) was added and the RM was stirred at RT for 1 h. The reaction mixture was quenched by addition of a sat. aq. NaHCO₃ solution and extracted with EtOAc (×2). The combined organic extracts were washed with a sat. aq. NaHCO₃ solution, dried (Na₂SO₄) and evaporated. The crude residue was purified by normal phase chromatography (eluent: (CH₂Cl₂/MeOH 8/2) in CH₂Cl₂ from 0 to 50%) to give the title compound as a grey foam. UPLC-MS-4: Rt=0.76 min; MS m/z [M+H]⁺ 481.4.

### Step 6: Benzyl (R)-6-(3-(2,2-dimethyl-4-(2-morpholinoethoxy)piperidin-1-yl)-5-methyl-1H-pyrazol-1-yl)-2-azaspiro[3.3]heptane-2-carboxylate

A solution of benzyl (R)-6-(3-(2,2-dimethyl-4-(2-oxoethoxy)piperidin-1-yl)-5-methyl-1H-pyrazol-1-yl)-2-azaspiro[3.3]heptane-2-carboxylate (Step 5, 1.00 g, 2.08 mmol) and morpholine (0.22 mL, 2.50 mmol) in dichloroethane (11 mL) was stirred at 0-5° C. for 10 min, then sodium triacetoxyborohydride (0.66 g, 3.12 mmol) was added and the reaction mixture was stirred at 0-5° C. for 30 min. The reaction mixture was poured into an aq. sat. NaHCO₃ solution and extracted with CH₂Cl₂ (×2). The combined organic extracts were dried (phase separator) and concentrated. The crude residue was purified by normal phase chromatography (eluent: MeOH in CH₂Cl₂ from 0 to 8%) to give the title compound as a brownish oil. UPLC-MS-4: Rt=0.66 min; MS m/z [M+H]⁺ 552.6.

### Step 7: Tert-butyl (R)-6-(3-(2,2-dimethyl-4-(2-morpholinoethoxy)piperidin-1-yl)-5-methyl-1H-pyrazol-1-yl)-2-azaspiro[3.3]heptane-2-carboxylate

To a solution of benzyl (R)-6-(3-(2,2-dimethyl-4-(2-morpholinoethoxy)piperidin-1-yl)-5-methyl-1H-pyrazol-1-yl)-2-azaspiro[3.3]heptane-2-carboxylate (Step 6, 1.00 g, 1.81 mmol) in THF (20 mL) was added (Boc)₂O (0.82 g, 3.80 mmol) and the mixture was evacuated and back-filled with nitrogen (×3). Pd-C 10% (0.20 g, 0.20 mmol) was added and the mixture was evacuated and back-filled with nitrogen (×2) then was evacuated and back-filled with hydrogen (×3). The reaction mixture was stirred at RT under hydrogen atmosphere (balloon) for 16 h. The RM was filtered over a pad of celite and washed with MeOH. The filtrate was concentrated and the crude residue was purified normal phase chromatography (eluent: (MeOH/CH₂Cl₂ 8/2) in CH₂Cl₂ from 0 to 100%) to give the title compound as a white foam. UPLC-MS-4: Rt=0.65 min; MS m/z [M+H]⁺ 518.5.

### Method-C78 for the preparation of Intermediate C78a and C78b: Tert-butyl 6-(3-(4-(3-methoxyazetidin-1-yl)-2,2-dimethylpiperidin-1-yl)-5-methyl-1H-pyrazol-1-yl)-2-azaspiro[3.3]heptane-2-carboxylate

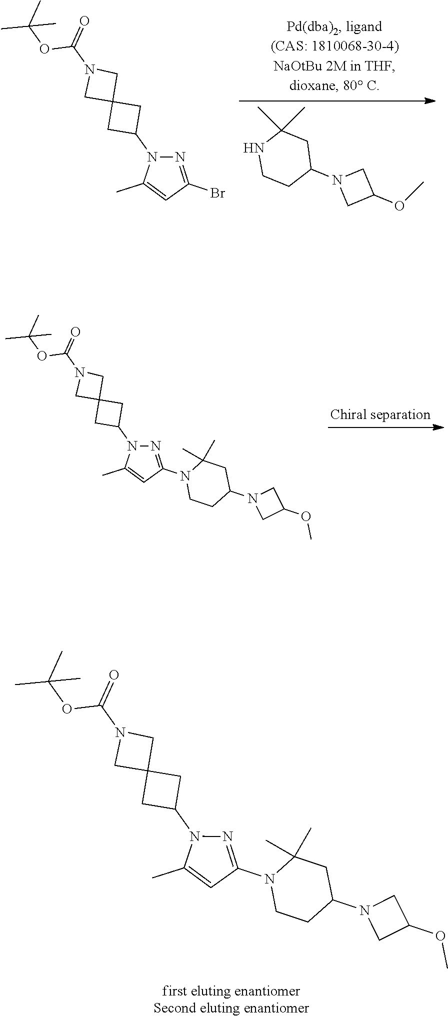

Tert-butyl 6-(3-bromo-5-methyl-1H-pyrazol-1-yl)-2-azaspiro[3.3]heptane-2-carboxylate (Intermediate C1, 680 mg, 1.91 mmol), 4-(3-methoxyazetidin-1-yl)-2,2-dimethylpiperidine (Intermediate A49, 672 mg, 2.86 mmol), Pd(dba)₂ (110 mg, 0.19 mmol) and bis(3,5-bis(trifluoromethyl)phenyl)(2′,6′-bis(dimethylamino)-3,6-dimethoxybiphenyl-2-yl)phosphine (CAS [1810068-30-4], 0.16 g, 0.21 mmol) were suspended in 1,4-dioxane (10 mL). NaOtBu (2M in THF, 3.82 mL, 7.63 mmol) was added, the vial was flushed with N₂ and the reaction mixture placed in a pre-heated oil bath at 80° C. for 1 h. Water was added and the mixture was extracted with EtOAc (×2). The combined organic extracts were washed with brine, dried (Na₂SO₄), filtered and concentrated. The crude residue was purified by normal phase chromatography (eluent: (CH₂Cl₂/10% MeOH) in CH₂Cl₂, 0 to 30%) and the purified fractions were neutralized by addition of a sat. aq. NaHCO₃ solution and extracted with EtOAc (×2) to give after evaporation under reduced pressure the title compound. The isomers were separated by chiral SFC (C-SFC-15; mobile phase: CO₂/[IPA+0.025% NH₃]: 60/40) to give the first eluting enantiomer of the title compound: Intermediate C78a; C-SFC-10 (mobile phase: CO₂/[IPA+0.1% NH₃]: 60/40): Rt=2.05 min, UPLC-MS-2a: Rt=0.84 min; MS m/z [M+H]⁺; 474.5 and the second eluting enantiomer of the title compound: Intermediate C78b: C-SFC-10 (mobile phase: CO₂/[IPA+0.1% NH₃]: 60/40): Rt=3.12 min, UPLC-MS-2a: Rt=0.84 min; MS m/z [M+H]⁺ 474.5.

Method-C78a: similar to Method-C78 except that Step 1 was performed in toluene instead of dioxane.

The following examples C79 to C83 were prepared using analogous methods to method-C78 from intermediates (in Step 1) described in the intermediates synthesis section A or commercially available.

| | | Method, | |
|---|---|---|---|
| | | intermediates and | |
| | | chiral separation | |
| Inter- | | conditions used and | |
| mediate | Structure | order of elution | Characterizing data |
| C79a/b | 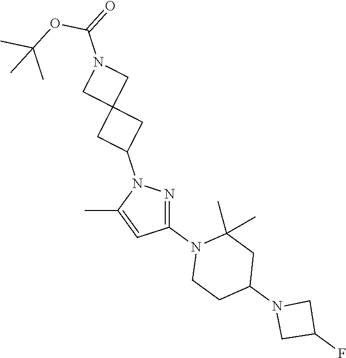 [SMILES: Cc1cc(N2CCC(N3CC(F)C3)CC2(C)C)nn1C1CC2(C1)CN(C(=O)OC(C)(C)C)C2] | Using Method-C78 from intermediate A50 and C-SFC-15 (mobile phase: CO₂/[IPA + 0.1% NH₂]: 75/25); Intermediate C79a = 1^{st} eluting isomer, Intermediate C79b = 2^{nd} eluting isomer | Intermediate C79a: UPLC-MS-2a: Rt = 0.84 min; MS m/z [M + H]⁺ 462.1; C-SFC-10 (mobile phase: CO₂/[IPA + 0.1% NH₃]: 75/25): Rt = 2.49 min, Intermediate C79b: C- SFC-10 (mobile phase: CO₂/[IPA + 0.1% NH₃]: 75/25): Rt = 3.18 min. |
| | Tert-butyl 6-(3-(4-(3- | | |
| | fluoroazetidin-1-yl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C80a/b | 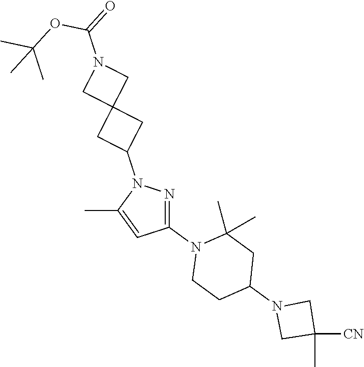 [SMILES: Cc1cc(N2CCC(N3CC(C)(C#N)C3)CC2(C)C)nn1C1CC2(C1)CN(C(=O)OC(C)(C)C)C2] | Using Method-C78 from intermediate A51 and C-SFC-16 (mobile phase: CO₂/[IPA + 0.1% NH₃]: 80/20); Intermediate C80a = 1^{st} eluting isomer, Intermediate C80b = 2^{nd} eluting isomer | Intermediate C80a: UPLC-MS-3: Rt = 0.81 min; MS m/z [M + H]⁺ 483.5; C-SFC-8 (mobile phase: CO₂ /[MeOH + 0.1% NH₃]: 80/20): Rt = 1.47 min, Intermediate C80b: C-SFC-8 (mobile phase: CO₂/[MeOH = 0.1% NH₃]: 80/20): Rt = 2.44 min. |
| | Tert-butyl 6-(3-(4-(3- | | |
| | cyano-3-methylazetidin-1- | | |
| | yl)-2,2-dimethylpiperidin- | | |
| | 1-yl)-5-methyl-1H-pyrazol- | | |
| | 1-yl)-2- | | |
| | azaspiro[3.3]heptane-2- | | |
| | carboxylate | | |
| C81a/b | 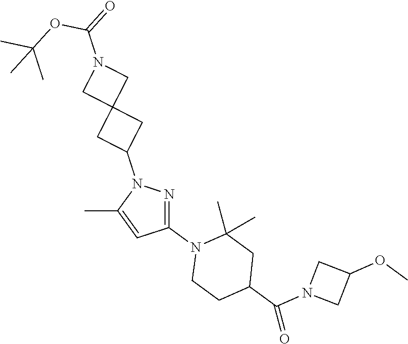 [SMILES: COC1CN(C(=O)C2CCN(c3cc(C)n(C4CC5(C4)CN(C(=O)OC(C)(C)C)C5)n3)C(C)(C)C2)C1] | Using Method Method-C78 from intermediate A82 and C-SFC-44 (mobile phase: CO₂/[CH₃OH + 0.025% NH₃]: 60/40); Intermediate C81a = 1^{st} eluting isomer, Intermediate C81b = 2^{nd} eluting isomer | Intermediate C81a: UPLC-MS-3: Rt = 0.87 min; MS m/z [M + H]⁺ 502.4; C-SFC-37 (mobile phase: CO₂/[MeOH + 0.025% NH₃]: 60/40): Rt = 1.43 min, Intermediate C81b: C-SFC-37 (mobile phase: CO₂/[MeOH + 0.025% NH₃]: 60/40): Rt = 2.93 min. |
| | Tert-butyl 6-(3-(4-(3- | | |
| | methoxyazetidine-1- | | |
| | carbonyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C82a/b | 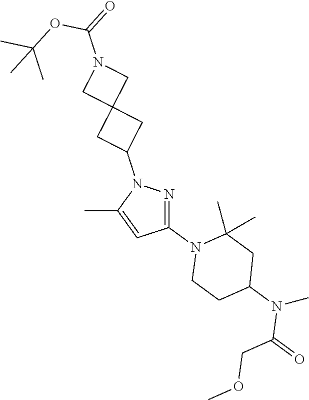 [SMILES: COCC(=O)N(C)C1CCN(c2cc(C)n(C3CC4(C3)CN(C(=O)OC(C)(C)C)C4)n2)C(C)(C)C1] | Using Method-C78 from intermediate A16 and C-SFC-23 (mobile phase: CO₂/IPA: 82/18); Intermediate C82a = 1^{st} eluting isomer, Intermediate C82b = 2^{nd} eluting isomer | Intermediate C82a: UPLC-MS-2a: Rt = 0.96 min; MS m/z [M + H]⁺ 490.4; C-SFC-3 (mobile phase: CO₂/IPA: 80/20): Rt = 1.10 min, Intermediate C82b: C- SFC-3 (mobile phase: CO₂/IPA: 80/20): Rt = 1.39 min. |
| | Tert-butyl 6-(3-(4-(2- | | |
| | methoxy-N- | | |
| | methylacetamido)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C83a/b | 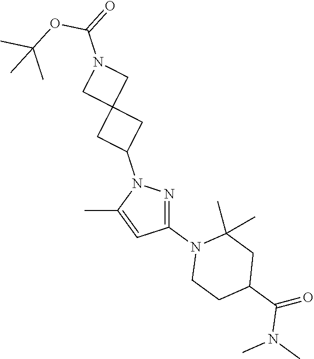 [SMILES: Cc1cc(N2CCC(C(=O)N(C)C)CC2(C)C)nn1C1CC2(C1)CN(C(=O)OC(C)(C)C)C2] | Using Method-C78 from intermediate A83 and C-SFC-18 (mobile phase: CO₂/[MeOH + 0.025% NH₃]: 60/40); Intermediate C83a = 1^{st} eluting isomer, Intermediate C83b = 2^{nd} eluting isomer | Intermediate C83a: UPLC-MS-4: Rt = 0.87 min; MS m/z [M + H]⁺ 460.5; C-SFC-19 (mobile phase: CO₂/[MeOH + 0.025% NH₃]: 60/40): Rt = 1.25 min, Intermediate C83b: C-SFC-19 (mobile phase: CO₂/[MeOH + 0.025% NH₃]: 60/40): Rt = 2.58 min. |
| | Tert-butyl 6-(3-(4- | | |
| | (dimethylcarbamoyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C84a/b | 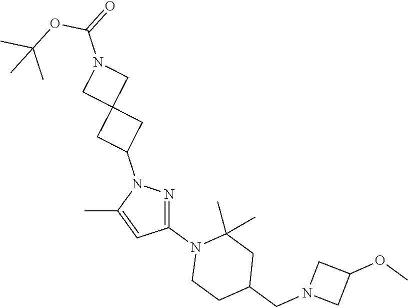 [SMILES: COC1CN(CC2CCN(c3cc(C)n(C4CC5(C4)CN(C(=O)OC(C)(C)C)C5)n3)C(C)(C)C2)C1] | Using Method-C78 from intermediate A80 and C-SFC-7 (mobile phase: CO₂/[IPA + 0.025% NH₃]: 82/18); Intermediate C84a = 1^{st} eluting isomer, Intermediate C84b = 2^{nd} eluting isomer | Intermediate C84a: UPLC-MS-4: Rt = 0.63 min; MS m/z [M + H]⁺ 488.5; C-SFC-8 (mobile phase: CO₂/[MeOH + 0.025% NH₃]: 82/18): Rt = 2.81 min, Intermediate C84b: C- SFC-8 (mobile phase: CO₂/[MeOH + 0.025% NH₃]: 82/18): Rt = 3.47 min. |
| | Tert-butyl 6-(3-(4-((3- | | |
| | methoxyazetidin-1- | | |
| | yl)methyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C85a/b | 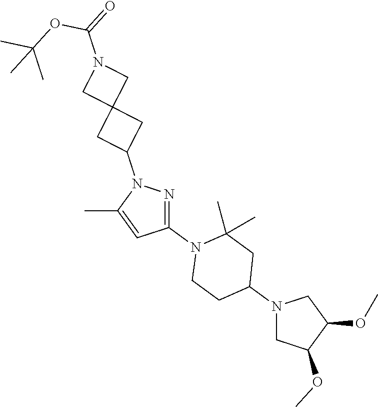 [SMILES: CO[C@H]1CN(C2CCN(c3cc(C)n(C4CC5(C4)CN(C(=O)OC(C)(C)C)C5)n3)C(C)(C)C2)C[C@H]1OC] | Using Method-C78 from intermediate A52 and C-SFC-6 (mobile phase: n- heptane/EtOH/MeOH: 95/2.5/2.5 + 0.01% DEA); Intermediate C85a = 1^{st} eluting isomer, Intermediate C85b = 2^{nd} eluting isomer | Intermediate C85a: UPLC-MS-4: Rt = 0.72 min; MS m/z [M + H]⁺ 518.5; C-SFC-13 (mobile phase: n- heptane/EtOH/MeOH/ DEA: 95/2.5/0.05): Rt = 11.6 min, Intermediate C85b: C- SFC-13 (mobile phase: n-heptane/EtOH/MeOH/ DEA: 95/2.5/2.5/0.05): Rt = 14.3 min. |
| | Tert-butyl 6-(3-(4- | | |
| | ((3S,4R)-3,4- | | |
| | dimethoxypyrrolidin-1-yl)- | | |
| | 2,2-dimethylpiperidin-1- | | |
| | yl)-5-methyl-1H-pyrazol-1- | | |
| | yl)-2- | | |
| | azaspiro[3.3]heptane-2- | | |
| | carboxylate | | |
| C86a/b | 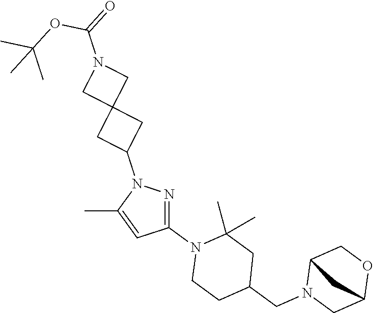 [SMILES: Cc1cc(N2CCC(CN3C[C@@H]4C[C@H]3CO4)CC2(C)C)nn1C1CC2(C1)CN(C(=O)OC(C)(C)C)C2] | Using Method-C78 from intermediate A81 and C-SFC-47 (mobile phase (CO₂/[MeOH + 0.1% DEA] 75:25); Intermediate C86a = 1^{st} eluting isomer, Intermediate C86b = 2^{nd} eluting isomer | Intermediate C86a: UPLC-MS-15: Rt = 3.76 min, MS m/z [M + H]⁺ 500.4; C-SFC-48 (mobile phase: CO₂/[MeOH + 0.1% DEA], gradient: 5 to 50%), Rt = 6.26 min, Intermediate C86b: C- SFC-48 (mobile phase: CO₂/[MeOH + 0.1% DEA], gradient: 5 to 50%), Rt = 6.87 min. |
| | Tert-butyl 6-(3-(4- | | |
| | (((1S,4S)-2-oxa-5- | | |
| | azabicyclo[2.2.1]heptan- | | |
| | 5-yl)methyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C87a/b | 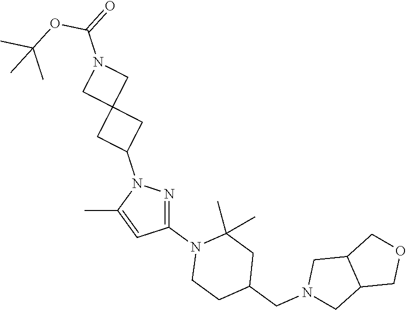 [SMILES: Cc1cc(N2CCC(CN3CC4COCC4C3)CC2(C)C)nn1C1CC2(C1)CN(C(=O)OC(C)(C)C)C2] | Using Method-C78 from intermediate A73 and C-SFC-27 (mobile phase (CO₂/[MeOH + 0.025% NH₃]: 87/13); Intermediate C87a = 1^{st} eluting isomer, Intermediate C87b = 2^{nd} eluting isomer | Intermediate 87a: UPLC-MS-4: Rt = 0.68 min, MS m/z [M + H]⁺ 514.4; C-SFC-6 (mobile phase: CO₂/[MeOH + 0.025% NH₃]: 85/15), Rt = 1.85 min, Intermediate C87b: UPLC-MS-4: Rt = 0.68 min, MS m/z [M + H]⁺ 514.4; C-SFC-6 (mobile phase: CO₂/[MeOH + 0.025% NH₃]: 85/15), Rt = 2.26 min. |
| | Tert-butyl 6-(3-(2,2- | | |
| | dimethyl-4-((tetrahydro- | | |
| | 1H-furo[3,4-c]pyrrol- | | |
| | 5(3H)-yl)methyl)piperidin- | | |
| | 1-yl)-5-methyl-1H-pyrazol- | | |
| | 1-yl)-2- | | |
| | azaspiro[3.3]heptane-2- | | |
| | carboxylate | | |
| C88a/b | 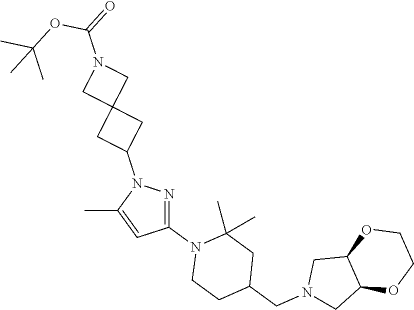 [SMILES: Cc1cc(N2CCC(CN3C[C@@H]4OCCO[C@@H]4C3)CC2(C)C)nn1C1CC2(C1)CN(C(=O)OC(C)(C)C)C2] | Using Method-C78a from intermediate A74 and C-SFC-7 (mobile phase (CO₂/[MeOH + 0.025% NH₃]: 73/27); Intermediate C88a = 1^{st} eluting isomer, Intermediate C88b = 2^{nd} eluting isomer | Intermediate C88a: UPLC-MS-4: Rt = 0.64 min, MS mz [M + H]⁺ 530.5; C-SFC-8 (mobile phase: CO₂/MeOH + 0.025% NH₃]: 73/27), Rt = 2.38 min, Intermediate C88b: UPLC-MS-4: Rt = 0.63 min, MS m/z [M + H]⁺ 530.4; C-SFC-8 (mobile phase: CO₂/[MeOH + 0.025% NH₃]: 73/27), Rt = 2.88 min. |
| | Tert-butyl 6-(3-(4- | | |
| | (((4aR,7aS)-hexahydro- | | |
| | 6H-[1,4]dioxino[2,3- | | |
| | c]pyrrol-6-yl)methyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C89a/b | 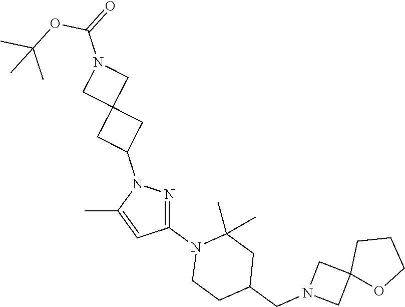 [SMILES: Cc1cc(N2CCC(CN3CC4(CCCO4)C3)CC2(C)C)nn1C1CC2(C1)CN(C(=O)OC(C)(C)C)C2] | Using Method-C78a from intermediate A75 and C-SFC- 18 (mobile phase (CO₂/[MeOH + 0.025% NH₃]: 65/35); tert-butyl (R)-6-(3-(4-((5-oxa-2-azaspiro [3.4]octan-2-yl)methyl)-2,2- dimethylpiperidin-1-yl)-5-methyl- 1H-pyrazol-1-yl)-2-azaspiro[3.3] heptane-2-carboxylate Int. C89a = 1^{st} eluting isomer, tert-butyl (S)-6-(3-(4-((5-oxa-2-azaspiro[3.4] octan-2-yl)methyl)-2,2- dimethylpiperidin-1-yl)-5-methyl- 1H-pyrazol-1-yl)-2-azaspiro[3.3] heptane-2-carboxylate Int. C89b = 2^{nd} eluting isomer | Intermediate C89a: UPLC-MS-4: Rt = 0.62 min, MS m/z [M + H]⁺ 514.5; C-SFC-46 (mobile phase: CO₂/[MeOH + 0.025% NH₃], 70/30): Rt = 2.59 min, Intermediate C90b: UPLC-MS-4: Rt = 0.62 min, MS m/z [M + H]⁺ 514.7; C-SFC-46 (mobile phase: CO₂/[MeOH + 0.025% NH₃], 70/30): Rt = 3.28 min. |
| | Tert-butyl 6-(3-(4-((5-oxa- | | |
| | 2-azaspiro[3.4]octan-2- | | |
| | yl)methyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C90a/b | 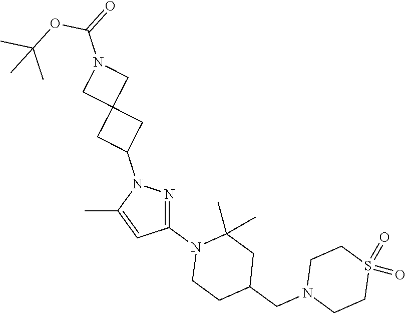 [SMILES: Cc1cc(N2CCC(CN3CCS(=O)(=O)CC3)CC2(C)C)nn1C1CC2(C1)CN(C(=O)OC(C)(C)C)C2] | Using Method-C78a from intermediate A79 and C-SFC-5 (mobile phase (CO₂/[IPA + 0.025% NH₃]: 70/30); Intermediate C90a = 1^{st} eluting isomer Intermediate C90b = 2^{nd} eluting isomer | Intermediate C90a: UPLC-MS-4: Rt = 0.75 min, MS m/z [M + H]⁺ 536.3; C-SFC-6 (mobile phase: CO₂/[MeOH + 0.025% NH₃]: 70/30), Rt = 1.08 min, Intermediate C90b: UPLC-MS-4: Rt = 0.75 min, MS m/z [M + H]⁺ 536.6; C-SFC-6 (mobile phase: CO₂/[MeOH + 0.025% NH₃]: 70/30), Rt = 1.36 min. |
| | Tert-butyl 6-(3-(4-((1,1- | | |
| | dioxidothiomorpholino) | | |
| | methyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |
| C91a/b | 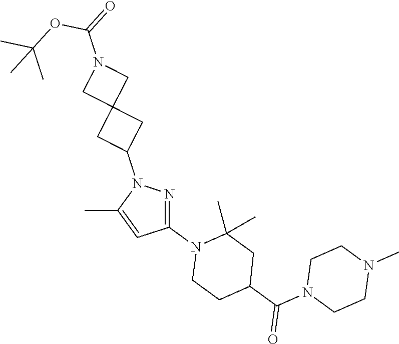 [SMILES: Cc1cc(N2CCC(C(=O)N3CCN(C)CC3)CC2(C)C)nn1C1CC2(C1)CN(C(=O)OC(C)(C)C)C2] | Using Method-C78a from intermediate A84 and C-SFC-52 (mobile phase (CO₂/[0.1% EtNH₂ in IPA/CH₃CN (50:50)]: gradient 5 to 50%): Intermediate C91a = 1^{st} eluting isomer, Intermediate C91b = 2^{nd} eluting isomer | Intermediate C91a: UPLC-MS-9: Rt = 1.02 min, MS m/z [M + H]⁺ 515.9, C-SFC-53 (mobile phase (CO₂/[0.1% EtNH₂ in IPA/CH₃CN (50:50)]: gradient 5 to 50%): Rt = 6.59 min, Intermediate C91b: UPLC-MS-9: Rt = 1.03 min, MS m/z [M + H]⁺ 515.9, C-SFC- 53 (mobile phase (CO₂/[0.1% EtNH₂ in IPA/CH₃CN (50:50)]: gradient 5 to 50%): Rt = 7.21 min. |
| | Tert-butyl 6-(3-(2,2- | | |
| | dimethyl-4-(4- | | |
| | methylpiperazine-1- | | |
| | carbonyl)piperidin-1-yl)-5- | | |
| | methyl-1H-pyrazol-1-yl)-2- | | |
| | azaspiro[3.3]heptane-2- | | |
| | carboxylate | | |
| C92a/b | 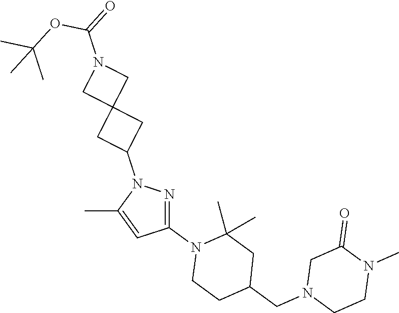 [SMILES: Cc1cc(N2CCC(CN3CCN(C)C(=O)C3)CC2(C)C)nn1C1CC2(C1)CN(C(=O)OC(C)(C)C)C2] | Using Method-C78a from Intermediate A76 and C-SFC-4 (mobile phase (CO₂/[MeOH + 0.1% NH₃] 80/20); Intermediate C92a = 1^{st} eluting isomer, Intermediate C92b = 2^{nd} eluting isomer | Intermediate C92a: UPLC-MS-4: Rt = 0.72 min; MS m/z [M + H]⁺ 515.4; C-SFC-3 (mobile phase: CO₂/[MeOH + 0.1% NH₃] 80/20): Rt = 1.49 min, Intermediate C92b: C- SFC-3 (mobile phase: CO₂/[MeOH + 0.1% NH₃] 80/20): Rt = 2.14 min. |
| | Tert-butyl 6-(3-(2,2- | | |
| | dimethyl-4-((4-methyl-3- | | |
| | oxopiperazin-1- | | |
| | yl)methyl)piperidin-1-yl)-5- | | |
| | methyl-1H-pyrazol-1-yl)-2- | | |
| | azaspiro[3.3]heptane-2- | | |
| | carboxylate | | |
| C93a/b | 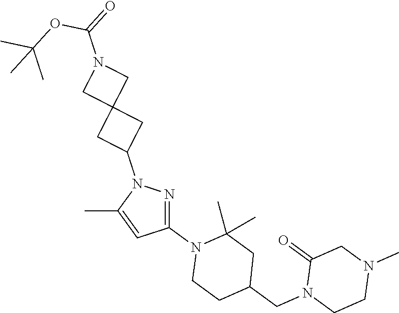 [SMILES: Cc1cc(N2CCC(CN3CCN(C)CC3=O)CC2(C)C)nn1C1CC2(C1)CN(C(=O)OC(C)(C)C)C2] | Using Method C-78a from Intermediate A77 and C-SFC-32 (mobile phase (CO₂/[MeOH + 0.1% NH₃] 75/25); Intermediate C93a = 1^{st} eluting isomer, Intermediate C93b = 2^{nd} eluting isomer | Intermediate C93a: UPLC-MS-4: Rt = 0.62 min; MS m/z [M + H]⁺ 515.4; C-SFC-19 (mobile phase: CO₂/[MeOH + 0.1% NH₃] 73/27): Rt = 3.70 min, Intermediate C93b: C- SFC-19 (mobile phase: CO₂/[MeOH + 0.1% NH₃] 73/27): Rt = 4.30 min. |
| | tert-butyl 6-(3-(2,2- | | |
| | dimethyl-4-((4-methyl-2- | | |
| | oxopiperazin-1- | | |
| | yl)methyl)piperidin-1-yl)-5- | | |
| | methyl-1H-pyrazol-1-yl)-2- | | |
| | azaspiro[3.3]heptane-2- | | |
| | carboxylate | | |

### Intermediate C94a and C94b: Tert-butyl 6-(3-(2,2-dimethyl-4-morpholinopiperidin-1-yl)-5-methyl-1H-pyrazol-1-yl)-2-azaspiro[3.3]heptane-2-carboxylate

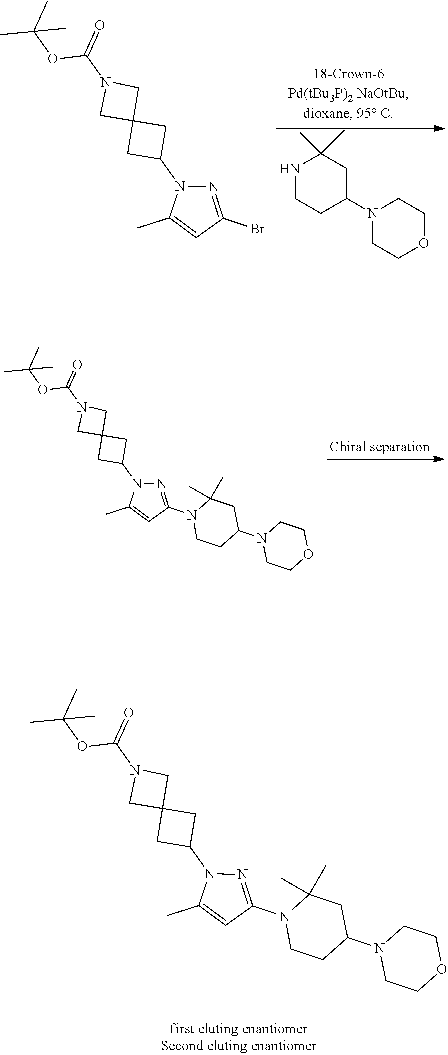 [SMILES: Cc1cc(Br)nn1C1CC2(C1)CN(C(=O)OC(C)(C)C)C2 ; CC1(C)CC(N2CCOCC2)CCN1 ; Cc1cc(N2CCC(N3CCOCC3)CC2(C)C)nn1C1CC2(C1)CN(C(=O)OC(C)(C)C)C2 ; Cc1cc(N2CCC(N3CCOCC3)CC2(C)C)nn1C1CC2(C1)CN(C(=O)OC(C)(C)C)C2]

Tert-butyl 6-(3-bromo-5-methyl-1H-pyrazol-1-yl)-2-azaspiro[3.3]heptane-2-carboxylate (Intermediate C1, 1.50 g, 4.20 mmol), 4-(2,2-dimethylpiperidin-4-yl)morpholine (Intermediate A53, 1.20 g, 5.46 mmol), 18-crown-6 (1.10 g, 4.20 mmol), NaOtBu (2M in THF, 2.40 g, 5.89 mmol) were suspended in 1,4-dioxane (40 mL) and degassed with nitrogen for 5 min. Pd(tBu₃P)₂ (0.21 g, 0.42 mmol) was added and the reaction mixture was heated to 95° C. in a screw capped vial for 2 h. After completion of the reaction, the reaction mixture was filtered through a pad of celite and the filtrate was concentrated under vacuum. The crude residue was purified by reverse phase chromatography (eluent: 0 to 100% CH₃CN in water containing 0.1% formic acid) to give the title product. The two enantiomers were separated by chiral HPLC (C-HPLC-25: mobile phase ([hexane+0.1% DEA]/[IPA:MeOH (50:50)]80:20); flow rate: 18 mL/min) to afford the first eluting isomer of the title compound: Intermediate C94a: UPLC-MS-5: Rt=1.50 min, MS m/z [M+H]⁺ 474.5, C-SFC-48 (mobile phase: CO₂/[MeOH+0.1% DEA], gradient 5 to 50%), Rt=5.79 min, and the second eluting isomer of the title compound: Intermediate C94b: C-SFC-48: (mobile phase: (CO₂/[MeOH+0.1% DEA], gradient 5 to 50%), Rt=6.10 min.

### Intermediates C95a and C95b: Tert-butyl 6-(4-iodo-5-methyl-3-(2,7,7-trimethyl-3-oxo-2,8-diazaspiro[4.5]decan-8-yl)-1H-pyrazol-1-yl)-2-azaspiro[3.3]heptane-2-carboxylate Isomer I and Isomer II

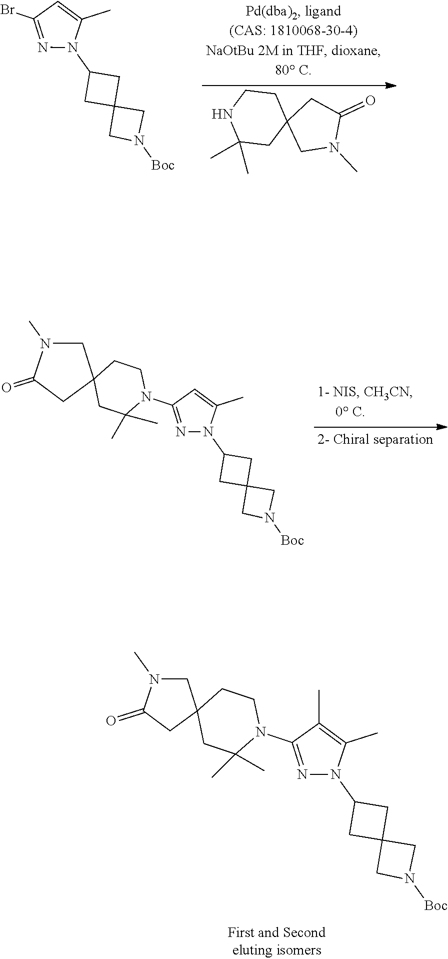 [SMILES: Cc1cc(Br)nn1C1CC2(C1)CN(C(=O)OC(C)(C)C)C2 ; CN1CC2(CCNC(C)(C)C2)CC1=O ; Cc1cc(N2CCC3(CC(=O)N(C)C3)CC2(C)C)nn1C1CC2(C1)CN(C(=O)OC(C)(C)C)C2 ; Cc1c(N2CCC3(CC(=O)N(C)C3)CC2(C)C)nn(C2CC3(C2)CN(C(=O)OC(C)(C)C)C3)c1C]

### Step: 1: Tert-butyl 6-(5-methyl-3-(2,7,7-trimethyl-3-oxo-2,8-diazaspiro[4.5]decan-8-yl)-1H-pyrazol-1-yl)-2-azaspiro[3.3]heptane-2-carboxylate

Tert-butyl 6-(3-bromo-5-methyl-1H-pyrazol-1-yl)-2-azaspiro[3.3]heptane-2-carboxylate (Intermediate C1, 1.25 g, 3.53 mmol) and 2,7,7-trimethyl-2,8-diazaspiro[4.5]decan-3-one (Intermediate A60, 0.90 g, 4.59 mmol) were dissolved in toluene (25 mL) and purged with nitrogen. NaOtBu (2.0M in THF, 8.80 mL, 17.7 mmol), bis(3,5-bis(trifluoromethyl)phenyl)(2′,6′-bis(dimethylamino)-3,6-dimethoxybiphenyl-2-yl)phosphine (CAS [1810068-30-4], 0.16 g, 0.21 mmol) and Pd(dba)₂ (0.12 g, 0.21 mmol) were added and the reaction mixture was heated to 90° C. for 24 h. After completion of the reaction, the RM was diluted with EtOAc and filtered through a pad of celite. The filtrate was concentrated under reduced pressure and the crude residue was purified by normal phase chromatography on neutral alumina (eluent: 0 to 2% MeOH in CH₂Cl₂) to give the title product. UPLC-MS-5: Rt=1.48 min, MS m/z [M+H]⁺ 472.6.

### Step: 2: Tert-butyl 6-(4-iodo-5-methyl-3-(2,7,7-trimethyl-3-oxo-2,8-diazaspiro[4.5]decan-8-yl)-1H-pyrazol-1-yl)-2-azaspiro[3.3]heptane-2-carboxylate Isomer I and Isomer II

To a solution of tert-butyl 6-(5-methyl-3-(2,7,7-trimethyl-3-oxo-2,8-diazaspiro[4.5]decan-8-yl)-1H-pyrazol-1-yl)-2-azaspiro[3.3]heptane-2-carboxylate (Step 1, 0.78 g, 1.66 mmol) in CH₃CN (5 mL) cooled at 0° C. was added NIS (0.39 g, 1.73 mmol) and the reaction mixture was stirred for 20 min. The RM was diluted with water and extracted with CH₂Cl₂. The combined organic layers were washed with water, brine, dried (Na₂SO₄), filtered and concentrated. The crude residue was purified reverse phase chromatography (eluent: 0 to 60% CH₃CN in water) to give the desired product. The enantiomers were separated by reverse phase chiral HPLC (C-HPLC-28 (mobile phase: MeOH+0.1% DEA isocratic 100%)) to give the first eluting isomer of the title compound: Intermediate C95a: UPLC-MS-5: Rt=2.11 min, MS m/z [M+H]⁺ 598.6; C-HPLC-29 (mobile phase: 0.1% DEA in MeOH, Isocratic 100%), Rt=7.86 min and the second eluting isomer of the title compound: Intermediate C95b: UPLC-MS-5: Rt=2.11 min, MS m/z [M+H]⁺ 598.6; C-HPLC-29 (mobile phase: 0.1% DEA in MeOH, Isocratic 100%), Rt=10.7 min.

### Intermediates C96a and C96b: Tert-butyl 6-(4-iodo-5-methyl-3-(4,8,8-trimethyl-1-oxa-4,9-diazaspiro[5.5]undecan-9-yl)-1H-pyrazol-1-yl)-2-azaspiro[3.3]heptane-2-carboxylate

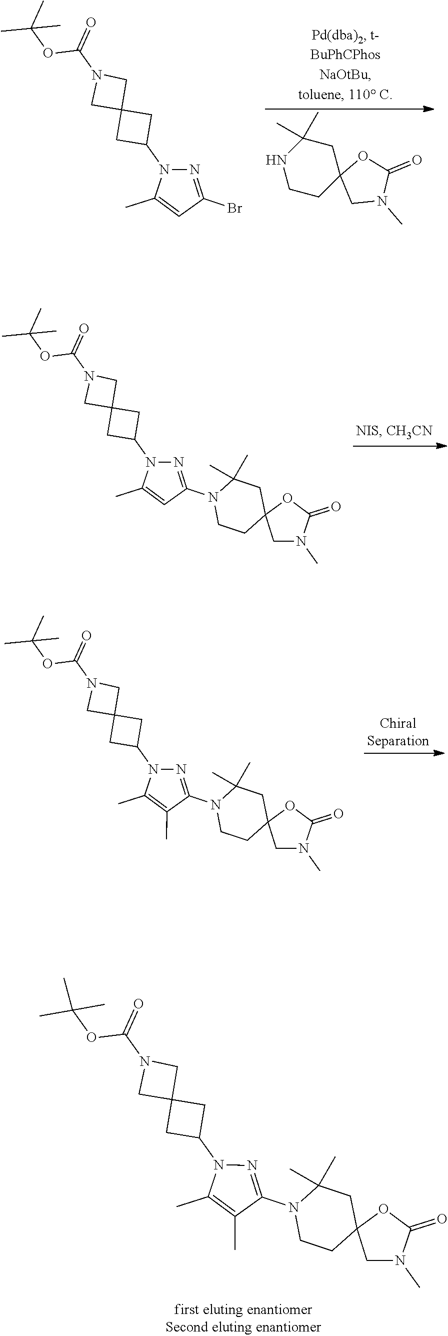 [SMILES: Cc1cc(Br)nn1C1CC2(C1)CN(C(=O)OC(C)(C)C)C2 ; CN1CC2(CCNC(C)(C)C2)OC1=O ; Cc1cc(N2CCC3(CN(C)C(=O)O3)CC2(C)C)nn1C1CC2(C1)CN(C(=O)OC(C)(C)C)C2 ; Cc1c(N2CCC3(CN(C)C(=O)O3)CC2(C)C)nn(C2CC3(C2)CN(C(=O)OC(C)(C)C)C3)c1C ; Cc1c(N2CCC3(CN(C)C(=O)O3)CC2(C)C)nn(C2CC3(C2)CN(C(=O)OC(C)(C)C)C3)c1C]

### Step 1: Tert-butyl 6-(5-methyl-3-(3,7,7-trimethyl-2-oxo-1-oxa-3,8-diazaspiro[4.5]decan-8-yl)-1H-pyrazol-1-yl)-2-azaspiro[3.3]heptane-2-carboxylate

A solution of tert-butyl 6-(3-bromo-5-methyl-1H-pyrazol-1-yl)-2-azaspiro[3.3]heptane-2-carboxylate (Intermediate C1, 1.20 g, 3.37) and 3,7,7-trimethyl-1-oxa-3,8-diazaspiro[4.5]decan-2-one (Intermediate A66, 0.86 g, 3.36 mmol) in toluene (48 mL) was degassed with argon for 10 min and NaOtBu (2M in THF, 0.97 g, 10.1 mmol), 18-crown 6 ether (0.89 g, 3.36 mmol), t-BuPhCPhos (0.20 g, 0.51 mmol) and Pd(dba)₂ (0.19 g, 0.34 mmol) were added. The reaction mixture was stirred at 110° C. for 4 h. Then the RM was quenched with water and extracted with EtOAc. The combined organic extracts were washed with a sat. aq. NaHCO₃ solution, brine, dried (Na₂SO₄), filtered and concentrated under vacuum. The crude residue was purified reverse phase chromatography (eluent: 70 to 80% CH₃CN in H₂O containing 0.025% NH₃) to give the title compound. UPLC-MS-5: Rt=1.56 min; MS m/z [M+H]⁺ 474.8.

### Step 2: Tert-butyl 6-(4-iodo-5-methyl-3-(3,7,7-trimethyl-2-oxo-1-oxa-3,8-diazaspiro[4.5]decan-8-yl)-1H-pyrazol-1-yl)-2-azaspiro[3.3]heptane-2-carboxylate

To a solution of tert-butyl 6-(5-methyl-3-(3,7,7-trimethyl-2-oxo-1-oxa-3,8-diazaspiro[4.5]decan-8-yl)-1H-pyrazol-1-yl)-2-azaspiro[3.3]heptane-2-carboxylate (Step 1, 1.10 g, 2.33 mmol) in CH₃CN (20 mL) at 0° C. under nitrogen atmosphere was added NIS (0.52 g, 2.33 mmol) and the reaction mixture was stirred at 0° C. for 30 min. The RM was quenched with water and extracted with CH₂Cl₂. The combined organic extracts were washed with a sat. aq. NaHCO₃ solution, brine, dried (Na₂SO₄), filtered and concentrated under vacuum. The crude residue was purified reverse phase chromatography (eluent: 90 to 100% CH₃CN in H₂O containing 0.1% NH₃) to give the title compound as pale yellow solid. The enantiomers were separated by chiral preparative HPLC (C-HPLC-27; mobile phase: [n-Hexane+0.1% DEA]/[MeOH/IPA+0.1% DEA 50/50]: 83/17) to give the first eluting isomer of the title compound: Intermediate C96a; C-HPLC-29 (mobile phase: [n-Hexane+0.1% DEA][MeOH/IPA 50/50]: 70/30): Rt=9.33 min, UPLC-MS-5: Rt=2.11 min; MS m/z [M+H]⁺ 600.8 and the second eluting isomer of the title compound: Intermediate C96b: C-HPLC-29 (mobile phase: [n-Hexane+0.1% DEA][MeOH/IPA 50/50]: 70/30): Rt=11.4 min, UPLC-MS-5: Rt=2.11 min; MS m/z [M+H]⁺ 600.8.

### Method-C97 for the preparation of C97a and C97b: Tert-butyl 6-(3-(4-((1R,5S)-3-oxa-8-azabicyclo[3.2.1]octan-8-yl)-2,2-dimethylpiperidin-1-yl)-5-methyl-1H-pyrazol-1-yl)-2-azaspiro[3.3]heptane-2-carboxylate

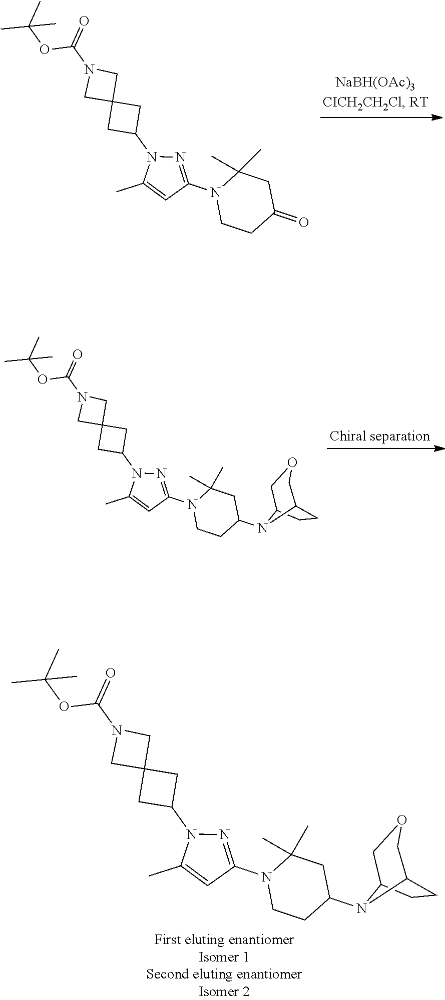 [SMILES: Cc1cc(N2CCC(=O)CC2(C)C)nn1C1CC2(C1)CN(C(=O)OC(C)(C)C)C2 ; Cc1cc(N2CCC(N3C4CCC3COC4)CC2(C)C)nn1C1CC2(C1)CN(C(=O)OC(C)(C)C)C2 ; Cc1cc(N2CCC(N3C4CCC3COC4)CC2(C)C)nn1C1CC2(C1)CN(C(=O)OC(C)(C)C)C2]

To a solution of (1R,5S)-3-oxa-8-azabicyclo[3.2.1]octane (1.01 g, 8.50 mmol) in DCE (60 mL) under argon atmosphere was added tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 2.40 g, 5.66 mmol) and the mixture was stirred at RT for 15 min. Then, sodium triacetoxyborohydride (3.60 g, 17.0 mmol) was added and the reaction mixture was stirred at RT for 20 h. The RM was then heated at 70° C. until completion. The RM was quenched by addition of a sat. aq. NaHCO₃ solution and extracted with EtOAc (×2). The combined organic extracts were washed with brine, dried (Na₂SO₄), filtered and evaporated. The crude residue was purified by normal phase chromatography (eluent: 0-10% MeOH in CH₂Cl₂) to give the title compound. The enantiomers were separated by chiral HPLC (C-HPLC-7; mobile phase: n-heptane/IPA 55/45+0.05% DEA) to give the first eluting isomer of the title compound: Intermediate C97a; C-HPLC-5 (mobile phase: n-heptane/IPA 55/45+0.05% DEA): Rt=5.85 min, UPLC-MS-4: Rt=0.75 min; MS m/z [M+H]⁺; 500.4 and the second eluting isomer of the title compound: Intermediate C97b: C-HPLC-5 (mobile phase: n-heptane/IPA 55/45+0.05% DEA): Rt=7.70 min, UPLC-MS-4: Rt=0.77 min; MS m/z [M+H]⁺ 500.4.

### Intermediates C98a and C98b: Tert-Butyl 6-(3-(4-(3,9-dioxa-7-azabicyclo[3.3.1]nonan-7-yl)-2,2-dimethylpiperidin-1-yl)-5-methyl-1H-pyrazol-1-yl)-2-azaspiro[3.3]heptane-2-carboxylate

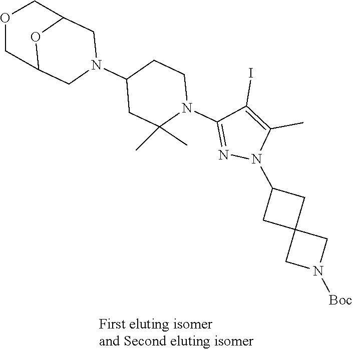 [SMILES: Cc1c(I)c(N2CCC(N3CC4COCC(C3)O4)CC2(C)C)nn1C1CC2(C1)CN(C(=O)OC(C)(C)C)C2]

### Step 1: Tert-Butyl 6-(3-(4-(3,9-dioxa-7-azabicyclo[3.3.1]nonan-7-yl)-2,2-dimethylpiperidin-1-yl)-5-methyl-1H-pyrazol-1-yl)-2-azaspiro[3.3]heptane-2-carboxylate

The title compound was prepared by a method similar to Method-C97 but using 3,9-dioxa-7-azabicyclo[3.3.1]nonane and CH₂Cl₂ instead of (1R,5S)-3-oxa-8-azabicyclo[3.2.1]octane and DCE respectively. UPLC-MS-4: Rt=0.73 min; MS m/z [M+H]⁺ 516.4.

### Step 2: Tert-butyl 6-(3-(4-(3,9-dioxa-7-azabicyclo[3.3.1]nonan-7-yl)-2,2-dimethylpiperidin-1-yl)-4-iodo-5-methyl-1H-pyrazol-1-yl)-2-azaspiro[3.3]heptane-2-carboxylate

The title compound was prepared by a method similar to Intermediates C101a and C101b Step 2 but using tert-butyl 6-(3-(4-(3,9-dioxa-7-azabicyclo[3.3.1]nonan-7-yl)-2,2-dimethylpiperidin-1-yl)-5-methyl-1H-pyrazol-1-yl)-2-azaspiro[3.3]heptane-2-carboxylate and THF instead of CH₃CN. The enantiomers were separated by chiral SFC (C-SFC-4 (mobile phase: CO₂/[IPA+0.025% NH₃] 85/15) to give the first eluting enantiomer of the title compound: Intermediate C98a: C-SFC-3 (mobile phase: CO₂/[MeOH+0.025% NH₃] 85/15), Rt=2.97 min; UPLC-MS-4: Rt=0.93 min, MS m/z [M+H]⁺ 642.5 and the second eluting enantiomer of the title compound: Intermediate C98b: C-SFC-3 (mobile phase: CO₂/[MeOH+0.025% NH₃] 85/15), Rt=4.10 min, UPLC-MS-4: Rt=0.93 min, MS m/z [M+H]⁺ 642.5.

### Intermediates C99a and C99b: Tert-butyl 6-(3-(2,2-dimethyl-4-((1R,4R)-5-(methylsulfonyl)-2,5-diazabicyclo[2.2.1]heptan-2-yl)piperidin-1-yl)-5-methyl-1H-pyrazol-1-yl)-2-azaspiro[3.3]heptane-2-carboxylate

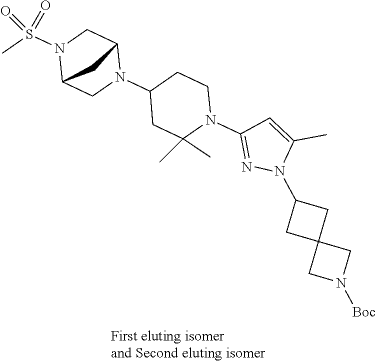 [SMILES: Cc1cc(N2CCC(N3C[C@H]4C[C@@H]3CN4S(C)(=O)=O)CC2(C)C)nn1C1CC2(C1)CN(C(=O)OC(C)(C)C)C2]

The title compound was prepared by a method similar to that of Intermediate Method-C97 using and (1R,4R)-2-(methylsulfonyl)-2,5-diazabicyclo[2.2.1]heptane (Intermediate A46) instead of (1R,5S)-3-oxa-8-azabicyclo[3.2.1]octane. The enantiomers were separated by chiral SFC (C-SFC-4 (mobile phase: CO₂/[IPA+0.1% Et₃N] 82/18) to give the first eluting isomer of the title compound: Intermediate C99a: C-SFC-3 (mobile phase: CO₂/[MeOH+0.025% NH₃] 85/15), Rt=1.66 min; UPLC-MS-4: Rt=0.68 min, MS m/z [M+H]⁺ 563.8 and the second eluting isomer of the title compound: Intermediate C99b: C-SFC-3 (mobile phase: CO₂/[MeOH+0.025% NH₃] 85/15), Rt=2.98 min, UPLC-MS-4: Rt=0.70 min, MS m/z [M+H]⁺ 563.6.

### Method-C100 for the preparation of Intermediates C100a and C100b: Tert-butyl 6-(3-(8-(bis(2-methoxyethyl)amino)-5-azaspiro[3.5]nonan-5-yl)-4-bromo-5-methyl-1H-pyrazol-1-yl)-2-azaspiro[3.3]heptane-2-carboxylate

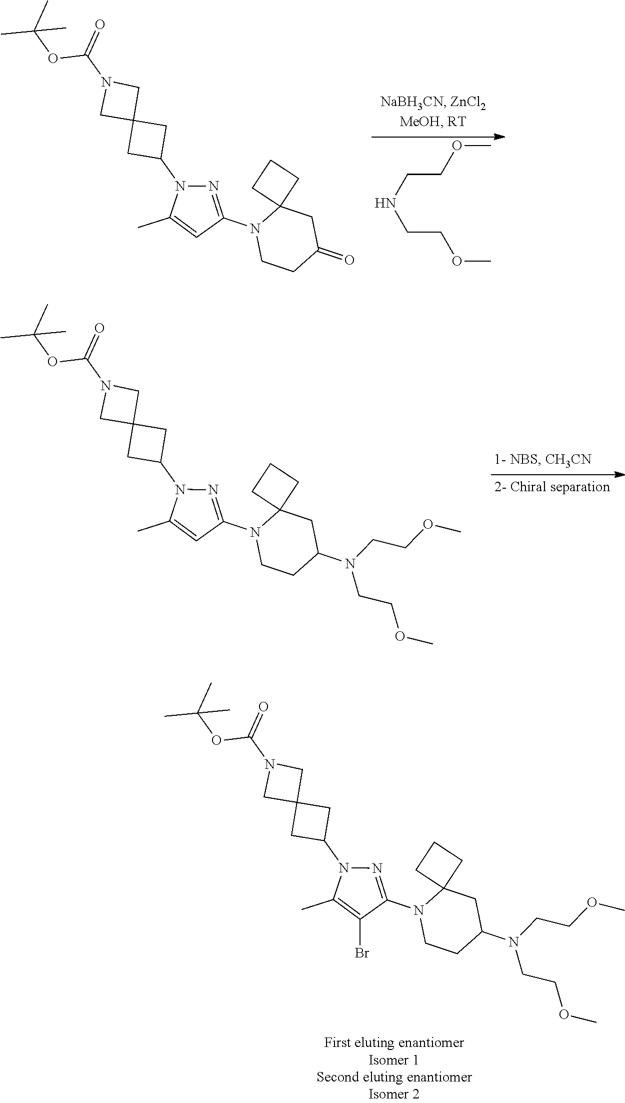

### Step 1: Tert-butyl 6-(3-(8-(bis(2-methoxyethyl)amino)-5-azaspiro[3.5]nonan-5-yl)-5-methyl-1H-pyrazol-1-yl)-2-azaspiro[3.3]heptane-2-carboxylate

In a Ace tube, to a solution of tert-butyl 6-(5-methyl-3-(8-oxo-5-azaspiro[3.5]nonan-5-yl)-1H-pyrazol-1-yl)-2-azaspiro[3.3]heptane-2-carboxylate (Intermediate C75, 1.00 g, 2.17 mmol) in MeOH (20 mL) was added under nitrogen atmosphere bis(2-methoxyethyl)amine (0.58 g, 4.34 mmol). The solution was cooled to 0° C., ZnCl₂ (0.03 g, 0.22 mmol) was added and the mixture was stirred for 10 min before addition of NaBH₃CN (0.55 g, 8.68 mmol). The reaction mixture was stirred at 50° C. for 16 h. Then was cooled to RT, poured into a sat. aq. NaHCO₃ solution and extracted with EtOAc (×2). The combined organic extracts were dried (phase separator) and concentrated. The crude residue was purified by normal phase chromatography (eluent: MeOH in CH₂Cl₂ from 0 to 5%) to give the title compound as a white foam. UPLC-MS-4: Rt=0.86 min; MS m/z [M+H]⁺ 532.4

### Step 2: Tert-butyl 6-(3-(8-(bis(2-methoxyethyl)amino)-5-azaspiro[3.5]nonan-5-yl)-4-bromo-5-methyl-1H-pyrazol-1-yl)-2-azaspiro[3.3]heptane-2-carboxylate

To an ice-cooled solution of tert-butyl 6-(3-(8-(bis(2-methoxyethyl)amino)-5-azaspiro[3.5]nonan-5-yl)-5-methyl-1H-pyrazol-1-yl)-2-azaspiro[3.3]heptane-2-carboxylate (Step 1, 920 mg, 1.73 mmol) in CH₃CN (12 mL) was added NBS (616 mg, 3.46 mmol) and the reaction mixture slowly allowed to react RT and stirred at RT for 16 h. The mixture reaction mixture was poured into a 10% Na₂S₂O₃ solution and extracted with EtOAc (2×). The combined organic extracts were washed with a sat. aq. NaHCO₃ solution, dried (phase separator) and concentrated. The crude residue was purified by normal phase chromatography (eluent: EtOAc in c-hexane 0 to 100%) to give the title compound. The enantiomers were separated by chiral HPLC (C-HPLC-7: mobile phase: n-heptane/EtOH 95/55+0.05% DEA) to give the first eluting isomer of the title compound: Intermediate C100a; C-HPLC-11 (mobile phase: n-heptane/EtOH 95/55+0.05% DEA): Rt=4.57 min, UPLC-MS-4: Rt=0.99 min; MS m/z [M+H]⁺; 610.4/612.4 and the second eluting isomer of the title compound: Intermediate C100b: C-HPLC-11 (mobile phase: n-heptane/EtOH 95/55+0.05% DEA): Rt=6.40 min, UPLC-MS-4: Rt=1.00 min; MS m/z [M+H]⁺ 610.3/612.3.

### Intermediates C101a and C101b: Tert-butyl 6-(4-iodo-5-methyl-3-(8-(methyl(oxetan-3-yl)amino)-5-azaspiro[3.5]nonan-5-yl)-1H-pyrazol-1-yl)-2-azaspiro[3.3]heptane-2-carboxylate

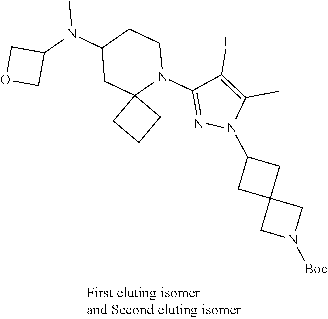 [SMILES: Cc1c(I)c(N2CCC(N(C)C3COC3)CC23CCC3)nn1C1CC2(C1)CN(C(=O)OC(C)(C)C)C2]

### Step 1: Tert-butyl 6-(5-methyl-3-(8-(methyl(oxetan-3-yl)amino)-5-azaspiro[3.5]nonan-5-yl)-1H-pyrazol-1-yl)-2-azaspiro[3.3]heptane-2-carboxyate

The title compound was prepared by a method similar to Method-C100 using N-methyloxetan-3-amine instead of bis(2-methoxyethyl)amine. UPLC-MS-5: Rt=1.50 min, MS m/z [M+H]⁺=486.3.

### Step 2: Tert-butyl 6-(4-iodo-5-methyl-3-(8-(methyl(oxetan-3-yl)amino)-5-azaspiro[3.5]nonan-5-yl)-1H-pyrazol-1-yl)-2-azaspiro[3.3]heptane-2-carboxyate

To a solution of tert-butyl 6-(5-methyl-3-(8-(methyl(oxetan-3-yl)amino)-5-azaspiro[3.5]nonan-5-yl)-1H-pyrazol-1-yl)-2-azaspiro[3.3]heptane-2-carboxylate (Step 1, 5.60 g, 12.0 mmol) in CH₃CN (280 mL) under nitrogen atmosphere was added NIS (2.59 g, 11.5 mmol) and the reaction mixture was stirred at 0° C. for 10 min. After completion of the reaction, the RM was diluted with water and extracted with EtOAc. The combined organic layer was washed with brine, dried (Na₂SO₄), filtered and concentrated under vacuum. The crude residue was purified by reverse phase chromatography (eluent: 40 to 50% CH₃CN in water containing 0.025% NH₃) to give the title product. The enantiomers were separated by chiral HPLC (C-HPLC-32: mobile phase: [hexane+0.1% DEA]/[IPA/MeOH 50/50]85/15) to give the first eluting isomer of the title compound: Intermediate C101a: C-SFC-53 (mobile phase: CO₂/[MeOH+0.1% DEA] 75:25), Rt=6.64 min; UPLC-MS-8: Rt=0.96 min, MS m/z [M+H]⁺ 612.4 and the second eluting isomer of the title compound: Intermediate C101b: C-SFC-53 (mobile phase: CO₂/[MeOH+0.1% DEA] 75:25), Rt=10.7 min, UPLC-MS-8: Rt=0.96 min, MS m/z [M+H]⁺ 612.4.

### Intermediates C102a and C102b: Tert-butyl 6-(3-(2,2-dimethyl-4-(methyl(oxetan-3-yl)amino)piperidin-1-yl)-5-methyl-1H-pyrazol-1-yl)-2-azaspiro[3.3]heptane-2-carboxylate

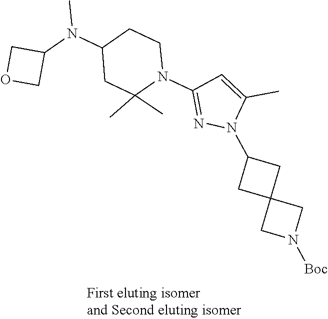 [SMILES: Cc1cc(N2CCC(N(C)C3COC3)CC2(C)C)nn1C1CC2(C1)CN(C(=O)OC(C)(C)C)C2]

The title compound was prepared by a method similar to Method-C100 Step 1 starting from tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74) and N-methyloxetan-3-amine. The enantiomers were separated by chiral SFC (C-SFC-47 (mobile phase: CO₂/[MeOH:CH₃CN (50:50)+0.1% DEA] 85/15) to give the first eluting isomer of the title compound: Intermediate C102a: C-SFC-48 (mobile phase: CO₂/[MeOH:CH₃CN (50:50)+0.1% DEA] 80/20), Rt=4.92 min; UPLC-MS-5: Rt=1.47 min, MS m/z [M+H]⁺ 474.5 and the second eluting isomer of the title compound: Intermediate C102b: C-SFC-48 (mobile phase: CO₂/[MeOH:CH₃CN (50:50)+0.1% DEA] 80/20), Rt=6.70 min, UPLC-MS-5: Rt=1.51 min, MS m/z [M+H]⁺ 474.9.

### Intermediates C103a and C103b: Tert-butyl 6-(5-methyl-3-(8-morpholino-5-azaspiro[3.5]nonan-5-yl)-1H-pyrazol-1-yl)-2-azaspiro[3.3]heptane-2-carboxyate

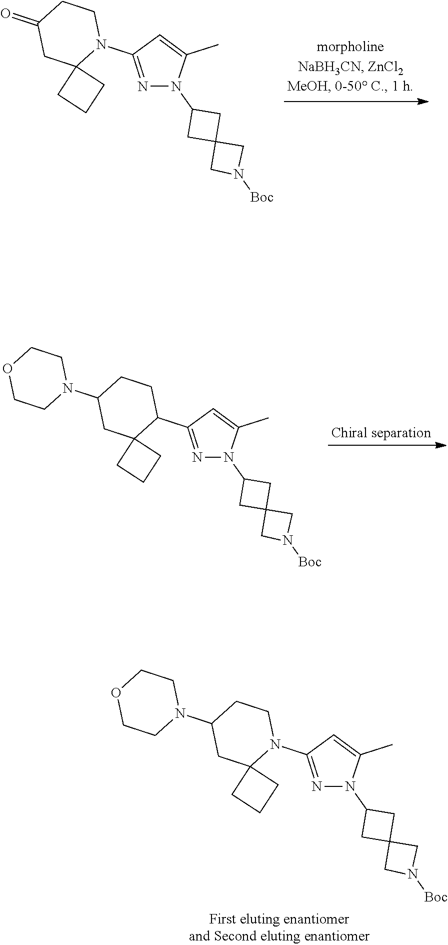 [SMILES: Cc1cc(N2CCC(=O)CC23CCC3)nn1C1CC2(C1)CN(C(=O)OC(C)(C)C)C2 ; Cc1cc(C2CCC(N3CCOCC3)CC23CCC3)nn1C1CC2(C1)CN(C(=O)OC(C)(C)C)C2 ; Cc1cc(N2CCC(N3CCOCC3)CC23CCC3)nn1C1CC2(C1)CN(C(=O)OC(C)(C)C)C2]

To a solution of tert-butyl 6-(5-methyl-3-(8-oxo-5-azaspiro[3.5]nonan-5-yl)-1H-pyrazol-1-yl)-2-azaspiro[3.3]heptane-2-carboxylate (Intermediate C75, 0.75 g, 1.80 mmol) in MeOH (5 mL) at 0° C. under nitrogen atmosphere, were added morpholine (0.31 g, 3.62 mmol) and ZnCl₂ (0.49 g, 3.62 mmol). The mixture was stirred for 5 min before the addition of NaBH₃CN (0.34 g, 5.41 mmol) and the reaction mixture was stirred at 50° C. for 1 h. After completion of the reaction, the RM was diluted with water and extracted with EtOAc (×2). The combined organic layer was washed with brine, dried (Na₂SO₄), filtered and concentrated under vacuum. The crude residue was purified by chromatography on neutral alumina (eluent: 0 to 5% MeOH in CH₂Cl₂) to afford the desired product. The enantiomers were separated by chiral SFC (C-SFC-54: mobile phase: CO₂/[MeOH+0.1% DEA]92:8) to give the first eluting isomer of the title compound: Intermediate C103a: C-SFC-55 (mobile phase: CO₂/[MeOH+0.1% DEA] gradient: 5 to 50%), Rt=3.83 min; UPLC-MS-5: Rt=1.57 min, MS m/z [M+H]⁺ 487.0 and the second eluting isomer of the title compound: Intermediate C103b: C-SFC-55 (mobile phase: CO₂/[MeOH+0.1% DEA] gradient: 5 to 50%), Rt=4.10 min; UPLC-MS-5: Rt=1.56 min, MS m/z [M+H]⁺ 487.0.

### Intermediates C104a and C104b: Tert-butyl 6-(3-(4-(3-hydroxy-3-methylazetidin-1-yl)-2,2-dimethylpiperidin-1-yl)-5-methyl-1H-pyrazol-1-yl)-2-azaspiro[3.3]heptane-2-carboxyate

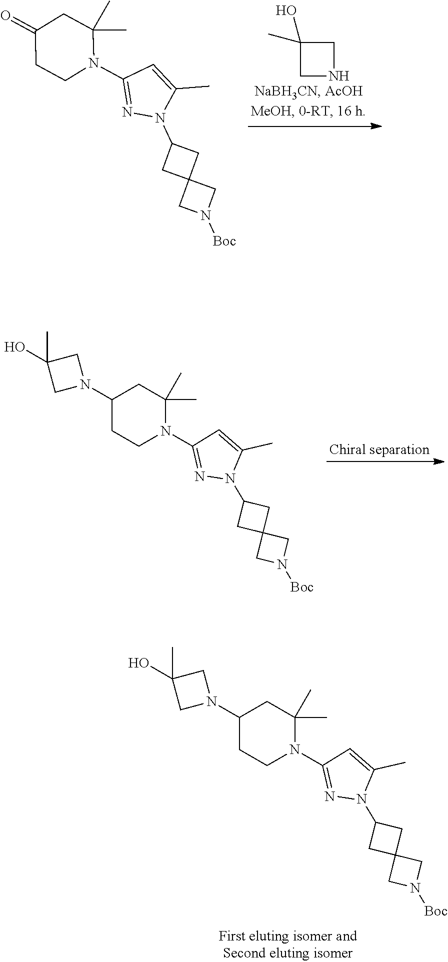

To a solution of tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 1.50 g, 3.72 mmol) and 3-methylazetidin-3-ol (0.92 g, 7.4 mmol) in methanol (30 mL) at 0° C. under nitrogen atmosphere was added acetic acid (0.022 g, 0.37 mmol). The mixture was stirred for 10 min before NaBH₃CN (2.30 g, 37.0 mmol) was added portionwise. The reaction mixture was stirred at RT for 16 h. After completion of the reaction, the RM was distilled and then was diluted with EtOAc, washed with a sat. solution of NaHCO₃, brine, dried (Na₂SO₄), filtered and concentrated under vacuum. The crude residue was purified chromatography on neutral alumina (eluent: 0 to 1% MeOH in CH₂Cl₂) to afford the title product. The enantiomers were separated by chiral SFC (C-SFC-47: mobile phase: CO₂/[CH₃CN/MeOH+0.1% DEA (50/50)] 85/15) to give the first eluting isomer of the title compound: Intermediate C104a: C-SFC-48 (mobile phase: CO₂/[CH₃CN/MeOH+0.1% DEA (50/50)] 70/30), Rt=6.63 min; UPLC-MS-5: Rt=1.52 min, MS m/z [M+H]⁺ 474.9 and the second eluting isomer of the title compound: Intermediate C104b: C-SFC-48 (mobile phase: CO₂/[CH₃CN/MeOH+0.1% DEA (50/50)] 70/30), Rt=9.01 min; UPLC-MS-5: Rt=1.45 min, MS m/z [M+H]⁺ 474.9.

### Intermediates C105a and C105b: Tert-butyl 6-(3-(4-((1S,4S)-2-oxa-5-azabicyclo[2.2.1]heptan-5-yl)-2,2-dimethylpiperidin-1-yl)-5-methyl-1H-pyrazol-1-yl)-2-azaspiro[3.3]heptane-2-carboxylate

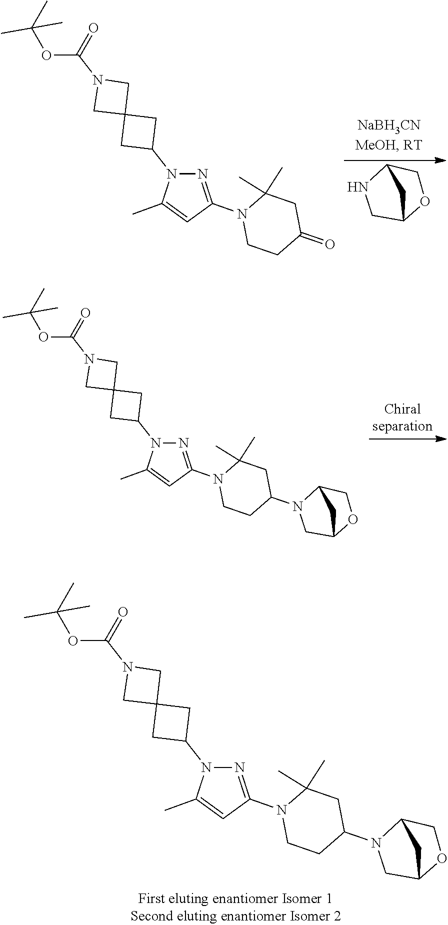 [SMILES: Cc1cc(N2CCC(=O)CC2(C)C)nn1C1CC2(C1)CN(C(=O)OC(C)(C)C)C2 ; C1O[C@@H]2CN[C@H]1C2 ; Cc1cc(N2CCC(N3C[C@@H]4C[C@H]3CO4)CC2(C)C)nn1C1CC2(C1)CN(C(=O)OC(C)(C)C)C2 ; Cc1cc(N2CCC(N3C[C@@H]4C[C@H]3CO4)CC2(C)C)nn1C1CC2(C1)CN(C(=O)OC(C)(C)C)C2]

To a solution of tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 1.70 g, 4.01 mmol) in MeOH (30 mL) was added under nitrogen atmosphere (1S,4S)-2-oxa-5-azabicyclo[2.2.1]heptane (746 mg, 6.02 mmol). The solution was stirred 15 min at RT and NaBH₃CN (756 mg, 12.0 mmol) was added. The RM was futher stirred at RT until completion of the reaction. The RM was poured into a sat. aq. NaHCO₃ solution and extracted with EtOAc (×2). The combined organic extracts were dried (Na₂SO₄), filtered and concentrated. The crude residue was purified by normal phase chromatography (eluent: MeOH in CH₂Cl₂ from 0 to 10%) to give the title compound as a white foam. The enantiomers were separated by chiral SFC (C-SFC-2: mobile phase: CO₂/[MeOH+0.025% NH₃] 85/15) to give the first eluting isomer of the title compound: Intermediate C105a; C-SFC-3 (mobile phase: CO₂/[MeOH+0.025% NH₃] 85/15): Rt=1.00 min, UPLC-MS-4: Rt=0.71 min; MS m/z [M+H]⁺ 486.4 and the second eluting isomer of the title compound: Intermediate C105b: C-SFC-3 (mobile phase: CO₂/[MeOH+0.025% NH₃] 85/15): Rt=1.56 min, UPLC-MS-4: Rt=0.71 min; MS m/z [M+H]⁺ 486.4.

### Intermediates C106a and C106b: Tert-butyl 6-(3-(2,2-dimethyl-4-(methyl((R)-tetrahydrofuran-3-yl)amino)piperidin-1-yl)-5-methyl-1H-pyrazol-1-yl)-2-azaspiro[3.3]heptane-2-carboxylate

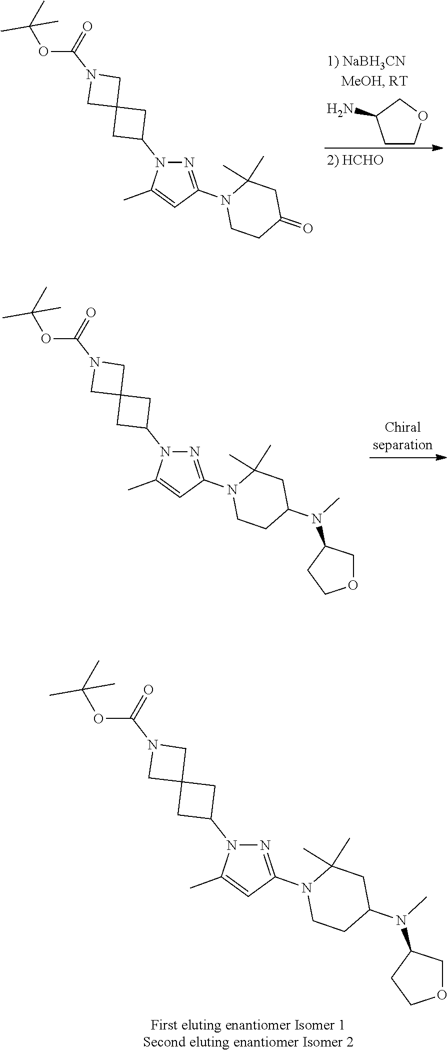 [SMILES: Cc1cc(N2CCC(=O)CC2(C)C)nn1C1CC2(C1)CN(C(=O)OC(C)(C)C)C2 ; N[C@@H]1CCOC1 ; Cc1cc(N2CCC(N(C)[C@@H]3CCOC3)CC2(C)C)nn1C1CC2(C1)CN(C(=O)OC(C)(C)C)C2 ; Cc1cc(N2CCC(N(C)[C@@H]3CCOC3)CC2(C)C)nn1C1CC2(C1)CN(C(=O)OC(C)(C)C)C2]

To a solution of tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 1.50 g, 3.65 mmol) in MeOH (25 mL) was added under nitrogen atmosphere (R)-3-aminotetrahydrofuran (0.48 g, 5.48 mmol). The solution was stirred 30 min at RT and NaBH₃CN (0.46 g, 7.30 mmol) was added. The RM was futher stirred at RT overnight and formaldehyde (25% in water, 1.50 mL, 20.2 mmol) was added. The RM was further stirred at RT for 64 h. Then was poured into a sat. aq. NaHCO₃ solution and extracted with EtOAc (×2). The combined organic extracts were dried (Na₂SO₄), filtered and concentrated. The crude residue was purified by normal phase chromatography (eluent: MeOH in CH₂Cl₂ from 0 to 10%) to give the title compound. The enantiomers were separated by chiral SFC (C-SFC-7: mobile phase: CO₂/[MeOH+0.025% NH₃] 70/30) to give the first eluting isomer of the title compound: Intermediate C106a; C-SFC-8 (mobile phase: CO₂/[MeOH+0.025% NH₃] 85/15): Rt=1.59 min, UPLC-MS-4: Rt=0.78 min; MS m/z [M+H]⁺ 488.4 and the second eluting isomer of the title compound: Intermediate C106b: C-SFC-8 (mobile phase: CO₂/[MeOH+0.025% NH₃] 85/15): Rt=1.97 min, UPLC-MS-4: Rt=0.78 min; MS m/z [M+H]⁺ 488.4.

### Intermediates C107a and C107b: Tert-butyl 6-(3-(4-(ethyl(oxetan-3-yl)amino)-2,2-dimethylpiperidin-1-yl)-5-methyl-1H-pyrazol-1-yl)-2-azaspiro[3.3]heptane-2-carboxylate

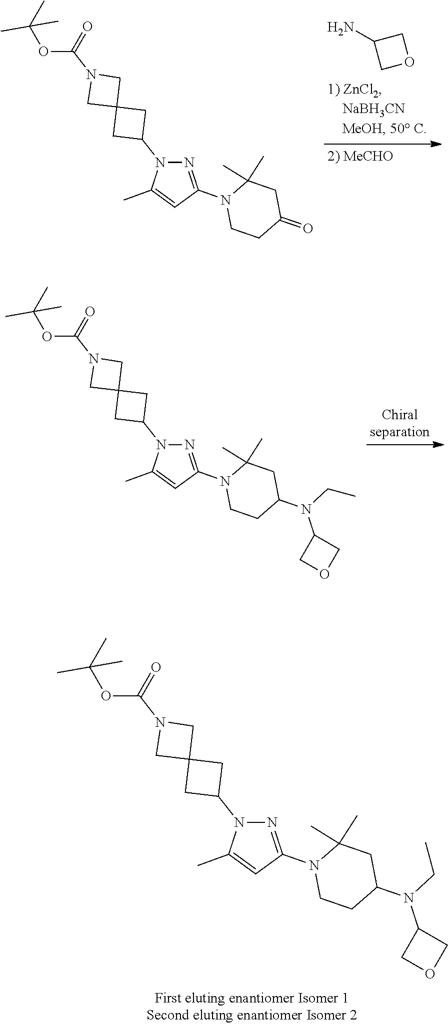

N-Oxetan-3-amine hydrochloride (1.39 g, 12.7 mmol) was added at 0° C. under nitrogen atmosphere to a solution of tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 2.56 g, 6.36 mmol) in MeOH (50 mL). ZnCl₂ (2.59 g, 19.1 mmol) was added and reaction mixture was stirred for 5 min prior to the addition of NaBH₃CN (1.2 g, 19.1 mmol). The reaction mixture was stirred at 50° C. for 16 h. Acetaldehyde (0.56 g, 12.7 mmol) was then added and the reaction mixture was further stirred at 50° C. for 1 h. After completion of the reaction, the RM was diluted with water and passed through celite bed. The filtrate was extracted with EtOAc (×3) and the combined organic extracts were washed with brine, dried (Na₂SO₄), filtered and concentrated under vacuum. The crude residue was purified by normal phase chromatography on neutral alumina (eluent: 0 to 5% MeOH in CH₂Cl₂) to give the title product. The enantiomers were separated by chiral SFC (C-SFC-47 (mobile phase: CO₂/[IPA:CH₃CN+0.1% DEA (50:50)] 85/15) to give the first eluting isomer of the title compound: Intermediate C107a; C-SFC-48 (mobile phase: CO₂/[IPA:CH₃CN+0.1% DEA (50:50)] 75/25), Rt=4.33 min, UPLC-MS-8: Rt=0.87 min, MS m/z [M+H]⁺ 488.6 and the second eluting isomer of the title compound: Intermediate C107b: C-SFC-48 (mobile phase: CO₂/[IPA:CH₃CN+0.1% DEA (50:50)] 75/25), Rt=5.35 min, UPLC-MS-8: Rt=0.88 min, MS m/z [M+H]⁺ 488.6.

### Intermediates C108a and C108b: Tert-butyl 6-(3-(4-hydroxy-2,2,4-trimethylpiperidin-1-yl)-5-methyl-1H-pyrazol-1-yl)-2-azaspiro[3.3]heptane-2-carboxylate

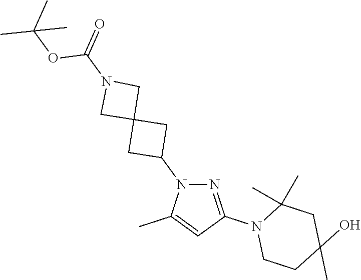 [SMILES: Cc1cc(N2CCC(C)(O)CC2(C)C)nn1C1CC2(C1)CN(C(=O)OC(C)(C)C)C2]

To a solution of tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 0.65 g, 1.61 mmol) in THF (8.10 mL) at 0° C. was added under nitrogen atmosphere methyl magnesium bromide (3M in Et₂O, 1.07 mL, 3.22 mmol). The reaction mixture was stirred at 0° C. for 1 h. The RM was quenched with cold water and extracted with CH₂Cl₂. The combined organic extracts were washed with brine, dried (Na₂SO₄), filtered and concentrated under vacuum. The crude residue was purified by reverse phase chromatography (eluent: from 45 to 50% CH₃CN in H₂O containing 0.1% Formic acid) to give the title compound. The enantiomers were separated by chiral preparative HPLC (C-HPLC-25; mobile phase: [n-Hexane+0.1% DEA]/IPA/MeOH 78/11/11); flow rate: 18 mL/min) to give the first eluting isomer of the title compound: Intermediate C108a; C-HPLC-26 (mobile phase: [n-Hexane+0.1% DEA]/[IPA:MeOH 50/50] 90/10): Rt=9.45 min, UPLC-MS-5: Rt=1.46 min; MS m/z [M+H]⁺ 419.5 and the second eluting isomer of the title compound: Intermediate C108b: C-HPLC-26 (mobile phase: [n-Hexane+0.1% DEA]/[IPA:MeOH 50/50] 90/10): Rt=11.4 min, UPLC-MS-5: Rt=1.46 min; MS m/z [M+H]⁺ 419.5.

### Intermediates C109a and C109b: Tert-butyl 6-(3-(4-(1H-imidazol-1-yl)-2,2-dimethylpiperidin-1-yl)-4-iodo-5-methyl-1H-pyrazol-1-yl)-2-azaspiro[3.3]heptane-2-carboxylate

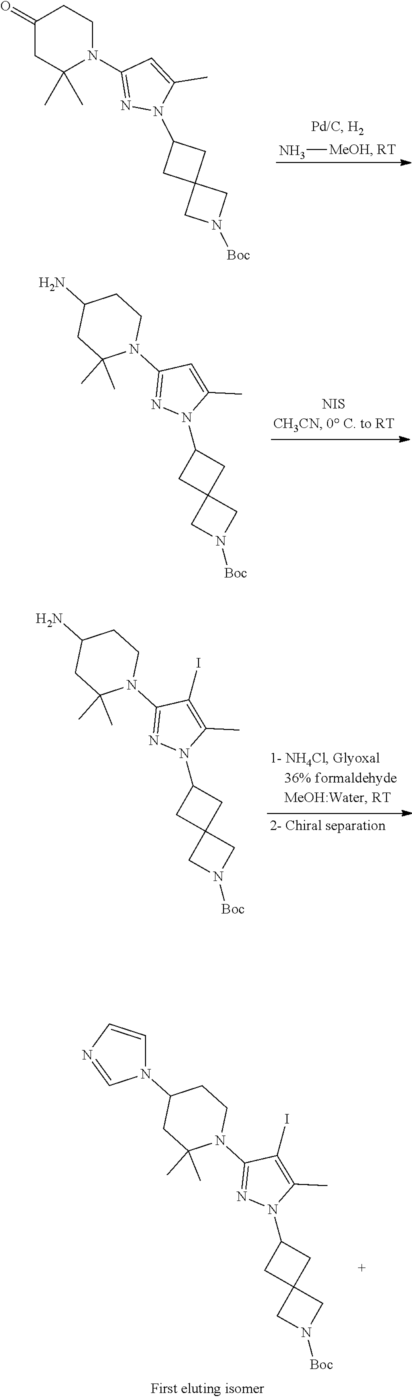

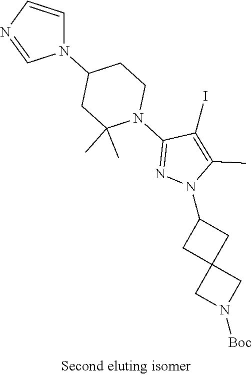 [SMILES: Cc1c(I)c(N2CCC(n3ccnc3)CC2(C)C)nn1C1CC2(C1)CN(C(=O)OC(C)(C)C)C2]

### Step 1: Tert-butyl 6-(3-(4-amino-2,2-dimethylpiperidin-1-yl)-5-methyl-1H-pyrazol-1-yl)-2-azaspiro[3.3]heptane-2-carboxylate

Tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 1.50 g, 3.70 mmol) was dissolved in a solution of NH₃ (4M in MeOH, 15 mL, 60 mmol). Pd/C 10% (0.80 g, 0.80 mmol) was added and the reaction mixture was stirred at RT under hydrogen (1 atmosphere) for 16 h. After completion of the reaction, the RM was filtered through a pad of celite and washed with MeOH. The filtrate was concentrated under vacuum to give the title product which was used in the next step without further purification. UPLC-MS-9: Rt=1.09 min, MS m/z [M+H]⁺ 404.3.

### Step 2: Tert-butyl 6-(3-(4-amino-2,2-dimethylpiperidin-1-yl)-4-iodo-5-methyl-1H-pyrazol-1-yl)-2-azaspiro[3.3]heptane-2-carboxylate

Tert-butyl 6-(3-(4-amino-2,2-dimethylpiperidin-1-yl)-5-methyl-1H-pyrazol-1-yl)-2-azaspiro[3.3]heptane-2-carboxylate (Step 1, 1.40 g, 3.50 mmol) was dissolved in CH₃CN (20 mL) and cooled to 0° C. under nitrogen atmosphere. NIS (0.78 g, 3.46 mmol) was added portionwise and the reaction mixture was stirred at RT for 1 h. After completion of the reaction, the RM was quenched with water and extracted with EtOAc. The combined organic layer was dried (Na₂SO₄), filtered and concentrated under vacuum. The crude residue was purified by reverse phase chromatography (eluent: 0 to 70% CH₃CN in water containing NH₃) to give the title product. UPLC-MS-9: Rt=1.26 min, MS m/z [M+H]⁺ 530.5.

### Step 3: Tert-butyl 6-(3-(4-(1H-imidazol-1-yl)-2,2-dimethylpiperidin-1-yl)-4-iodo-5-methyl-1H-pyrazol-1-yl)-2-azaspiro[3.3]heptane-2-carboxylate

Tert-butyl 6-(3-(4-amino-2,2-dimethylpiperidin-1-yl)-4-iodo-5-methyl-1H-pyrazol-1-yl)-2-azaspiro[3.3]heptane-2-carboxylate (Step 2, 0.20 g, 0.40 mmol) and NH₃ (25% in water, 0.03 mL, 0.40 mmol) were added in MeOH (2 mL) and the mixture was cooled to 0° C. Glyoxal (40% in water, 0.054 mL, 0.38 mmol) and formaldehyde (38% in water, 0.03 mL, 0.38 mmol) were added and the reaction mixture was stirred at 60° C. for 5 h. After completion of the reaction, water was added and the mixture was extracted with EtOAc. The combined organic layer was dried (Na₂SO₄), filtered and concentrated under vacuum. The crude residue was purified by reverse phase chromatography (eluent: 0 to 70% CH₃CN in water containing 0.1% NH₃) to give the title product. The enantiomers were separated by chiral preparative HPLC (C-HPLC-32 (mobile phase: [n-Hexane+0.1% DEA]/[IPA:MeOH (50:50)] 85/15) to give the first eluting isomer of the title compound: Intermediate C109a:

C-HPLC-33 (mobile phase: [n-Hexane+0.1% DEA]/[IPA:MeOH 50/50] gradient 20 to 70%): Rt=7.72 min, UPLC-MS-9: Rt=1.31 min, MS m/z [M+H]⁺ 581.6 and the second eluting isomer of the title compound: Intermediate C109b: C-HPLC-33 (mobile phase: [n-Hexane+0.1% DEA]/[IPA:MeOH 50/50] gradient 20 to 70%): Rt=9.85 min, UPLC-MS-9: Rt=1.31 min, MS m/z [M+H]⁺ 581.6.

### Method-C110 for the preparation of Intermediates C110a and C110b: Tert-butyl 6-(4-(5-chloro-6-methyl-1H-indazol-4-yl)-5-methyl-3-(8-oxo-5-azaspiro[3.5]nonan-5-yl)-1H-pyrazol-1-yl)-2-azaspiro[3.3]heptane-2-carboxylate

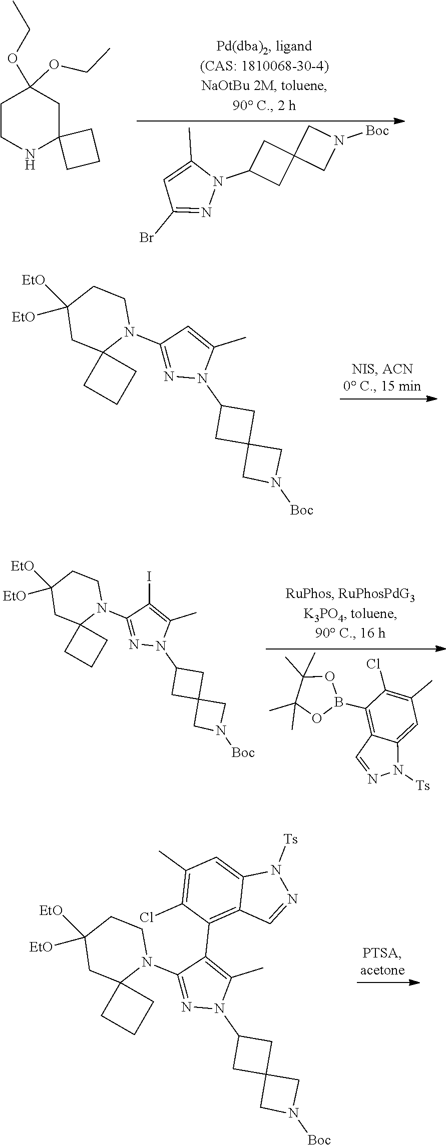

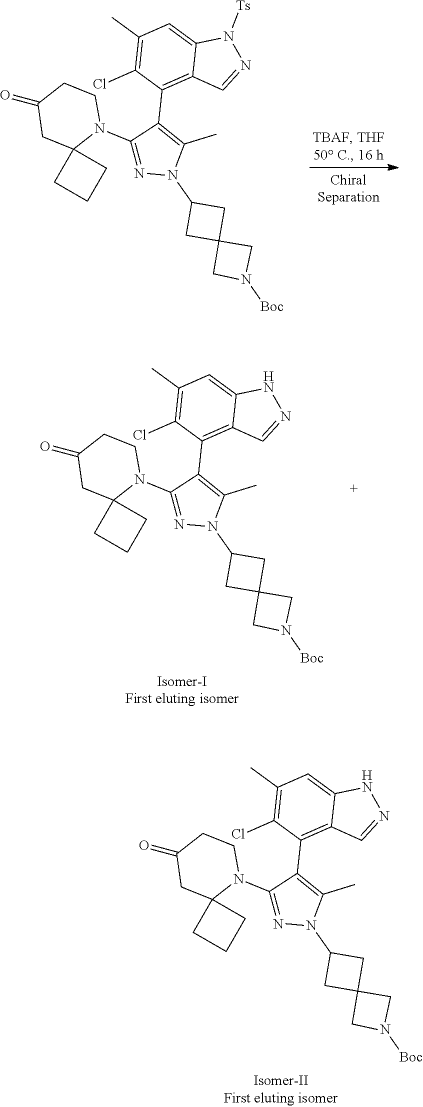 [SMILES: Cc1ccc(S(=O)(=O)n2ncc3c(-c4c(N5CCC(=O)CC56CCC6)nn(C5CC6(C5)CN(C(=O)OC(C)(C)C)C6)c4C)c(Cl)c(C)cc32)cc1 ; Cc1cc2[nH]ncc2c(-c2c(N3CCC(=O)CC34CCC4)nn(C3CC4(C3)CN(C(=O)OC(C)(C)C)C4)c2C)c1Cl ; Cc1cc2[nH]ncc2c(-c2c(N3CCC(=O)CC34CCC4)nn(C3CC4(C3)CN(C(=O)OC(C)(C)C)C4)c2C)c1Cl]

### Step 1: Tert-butyl 6-(3-(8,8-diethoxy-5-azaspiro[3.5]nonan-5-yl)-5-methyl-1H-pyrazol-1-yl)-2-azaspiro[3.3]heptane-2-carboxylate

A solution of tert-butyl 6-(3-bromo-5-methyl-1H-pyrazol-1-yl)-2-azaspiro[3.3]heptane-2-carboxylate (Intermediate C1, 5.00 g, 14.1 mmol) and 8,8-diethoxy-5-azaspiro[3.5]nonane (A48, 2.99 g, 14.1 mmol) in dry toluene (120 mL) was degassed for 5 min. Pd(dba)₂ (0.81 g, 1.40 mmol), bis(3,5-bis(trifluoromethyl)phenyl)(2′,6′-bis(dimethylamino)-3,6-dimethoxybiphenyl-2-yl)phosphine (CAS [1810068-30-4], 1.17 g, 1.55 mmol) and NaOtBu (2.0 M in THF, 56 mL, 112.4 mmol) was added and the reaction mixture and was heated to 90° C. for 2 h in a screw capped vessel. After completion of the reaction, the RM was diluted with water and extracted by EtOAc (×2). The combined organic layer was washed with brine, dried (Na₂SO₄), filtered and concentrated. The crude residue was purified by chromatography on neutral alumina (eluent: 0.5 to 1% MeOH in CH₂Cl₂) to give the title compound as a pale yellow oil. UPLC-MS-5: Rt=2.40 min, MS m/z [M+H]⁺ 489.5.

### Step 2: Tert-butyl 6-(3-(8,8-diethoxy-5-azaspiro[3.5]nonan-5-yl)-4-iodo-5-methyl-1H-pyrazol-1-yl)-2-azaspiro[3.3]heptane-2-carboxylate

To a solution of tert-butyl 6-(3-(8,8-diethoxy-5-azaspiro[3.5]nonan-5-yl)-5-methyl-1H-pyrazol-1-yl)-2-azaspiro[3.3]heptane-2-carboxylate (Step 1, 1.10 g, 2.30 mmol) in CH₃CN (15 mL) at 0° C. under nitrogen atmosphere was added portionwise NIS (0.51 g, 2.25 mmol) and the reaction mixture was stirred at 0° C. for 15 min. After completion of the reaction, the RM was diluted water and extracted with EtOAc. The combined organic layer was washed with water, sodium thiosulphate 10% solution, dried (Na₂SO₄), filtered and concentrated under vacuum to give the title compound which was used in the next step without further purification. UPLC-MS-6: Rt=3.06 min, MS m/z [M−OEt+H]⁺569.5.

### Step 3: Tert-butyl 6-(4-(5-chloro-6-methyl-1-tosyl-1H-indazol-4-yl)-3-(8,8-diethoxy-5-azaspiro[3.5]nonan-5-yl)-5-methyl-1H-pyrazol-1-yl)-2-azaspiro[3.3]heptane-2-carboxylate

Tert-butyl 6-(3-(8,8-diethoxy-5-azaspiro[3.5]nonan-5-yl)-4-iodo-5-methyl-1H-pyrazol-1-yl)-2-azaspiro[3.3]heptane-2-carboxylate (Step 2, 8.62 g, 14.0 mmol), 5-chloro-6-methyl-4-(4,4,5,5-tetramethyl-1,3,2-dioxaborolan-2-yl)-1-tosyl-1H-indazole (Intermediate D4, 8.14 g, 18.2 mmol) and K₃PO₄ (2.0 M in water, 21 mL, 42 mmol) were added in toluene (43 mL). The reaction mixture was degassed with nitrogen for 5 min. Ruphos (1.00 g, 2.10 mmol) and RuPhos-Pd-G3 (1.20 g, 1.4 mmol) were added and the reaction mixture was stirred at 90° C. for 16 h. After completion of the reaction, the RM was diluted with water and extracted with EtOAc. The combined organic layer was washed with brine, dried (Na₂SO₄), filtered and concentrated to afford the desired product which was used in the next step without further purification. UPLC-MS-13: Rt=5.91 min, MS m/z [M-OEt]⁺761/763, [M+H]⁺ 807/809.

### Step 4: Tert-butyl 6-(4-(5-chloro-6-methyl-1-tosyl-1H-indazol-4-yl)-5-methyl-3-(8-oxo-5-azaspiro[3.5]nonan-5-yl)-1H-pyrazol-1-yl)-2-azaspiro[3.3]heptane-2-carboxylate

Tert-butyl 6-(4-(5-chloro-6-methyl-1-tosyl-1H-indazol-4-yl)-3-(8,8-diethoxy-5-azaspiro[3.5]nonan-5-yl)-5-methyl-1H-pyrazol-1-yl)-2-azaspiro[3.3]heptane-2-carboxylate (Step 3, 16.0 g, 20 mmol) was dissolved in acetone (100 mL) and cooled to 0° C. p-Toluene sulfonic acid (0.94 g, 1.98 mmol) was added and the reaction mixture was allowed to stir at RT for 16 h. After completion of the reaction, the RM was quenched with ice cold water and extracted with EtOAc. The combined organic layer was washed with brine, dried (Na₂SO₄), filtered and concentrated. The crude residue was further purified by chromatography on neutral alumina (eluent: 45 to 55% EtOAc in hexane) to obtain the desired product. UPLC-MS-5: Rt=2.47 min, MS m/z [M+H]⁺ 734.6.

### Step 5: Tert-butyl 6-(4-(5-chloro-6-methyl-1H-indazol-4-yl)-5-methyl-3-(8-oxo-5-azaspiro[3.5]nonan-5-yl)-1H-pyrazol-1-yl)-2-azaspiro[3.3]heptane-2-carboxylate

Tert-butyl 6-(4-(5-chloro-6-methyl-1-tosyl-1H-indazol-4-yl)-5-methyl-3-(8-oxo-5-azaspiro[3.5]nonan-5-yl)-1H-pyrazol-1-yl)-2-azaspiro[3.3]heptane-2-carboxylate (Step 4, 5.80 g, 7.90 mmol) was dissolved in THF (200 mL). Tetrabutyl ammonium fluoride (1M in THF, 16.0 mL, 16.0 mmol) was added and the reaction mixture was heated at 55° C. for 16 h. After completion of the reaction, the RM was quenched with ice cold water and extracted with EtOAc. The combined organic layer was washed with brine, dried (Na₂SO₄), filtered and concentrated. The crude residue was purified by chromatography on neutral alumina (eluent: 0 to 4% MeOH in CH₂Cl₂) to give the title product. The two enantiomers were separated by chiral SFC C-SFC-50 (mobile phase: CO₂/MeOH 87/13) to give the first eluting isomer of the title compound: Intermediate C110a; C-SFC-51 (mobile phase: CO₂/MeOH, gradient 5 to 50%), Rt=5.0 min, UPLC-MS-5: Rt=2.21 min, MS m/z [M+H]⁺ 579.6/581.6 and the second eluting isomer of the title compound: Intermediate C110b: C-SFC-51: Mobile Phase: (CO₂/MeOH, gradient 5 to 50%), Rt=5.4 min, UPLC-MS-6: Rt=2.20 min, MS m/z [M+H]⁺ 579.6/581.6.

### Intermediates C111a and C111b: Tert-butyl 6-(4-(5-chloro-6-methyl-1H-indazol-4-yl)-3-(2,2-dimethyl-4-oxopiperidin-1-yl)-5-methyl-1H-pyrazol-1-yl)-2-azaspiro[3.3]heptane-2-carboxylate

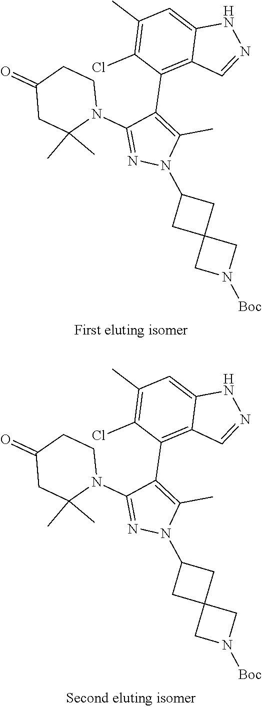 [SMILES: Cc1cc2[nH]ncc2c(-c2c(N3CCC(=O)CC3(C)C)nn(C3CC4(C3)CN(C(=O)OC(C)(C)C)C4)c2C)c1Cl ; Cc1cc2[nH]ncc2c(-c2c(N3CCC(=O)CC3(C)C)nn(C3CC4(C3)CN(C(=O)OC(C)(C)C)C4)c2C)c1Cl]

The title compounds were prepared by a method similar to Method-C110 using 4,4-diethoxy-2,2-dimethylpiperidine (intermediate A47) instead of 8,8-diethoxy-5-azaspiro[3.5]nonane (intermediate A48) in Step 1 and THF instead of CH₃CN in step 2. The two enantiomers were separated by chiral SFC C-SFC-4 (mobile phase: CO₂/[IPA+0.1% Et₃N] 75/25) to give the first eluting isomer of the title compound: Intermediate C111a; C-SFC-3 (mobile phase: CO₂/[IPA+0.1% Et₃N] 75/25): Rt=1.39 min; UPLC-MS-4: Rt=1.31 min; MS m/z [M+H]⁺ 567.3/569.3 and the second eluting isomer of the title compound: Intermediate C111b: C-SFC-3 (mobile phase: CO₂/[IPA+0.1% Et₃N] 75/25): Rt=2.24 min; UPLC-MS-4: Rt=1.31 min; MS m/z [M+H]⁺ 567.3/569.3.

### Intermediate C112: Tert-butyl 6-(4-(5-chloro-6-methyl-1H-indazol-4-yl)-3-(4-(ethyl(oxetan-3-yl)amino)-2,2-dimethylpiperidin-1-yl)-5-methyl-1H-pyrazol-1-yl)-2-azaspiro[3.3]heptane-2-carboxylate

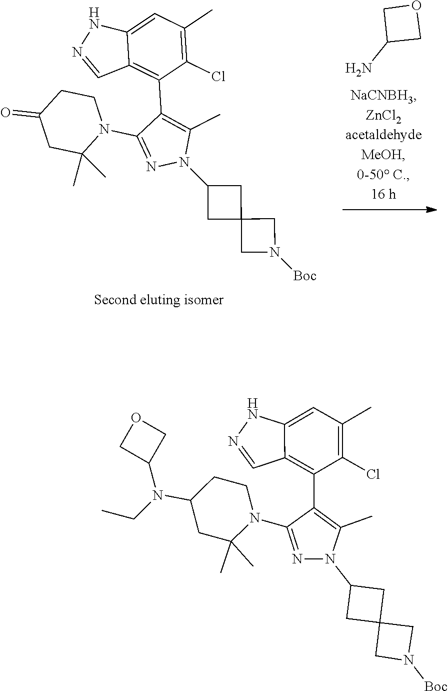 [SMILES: Cc1cc2[nH]ncc2c(-c2c(N3CCC(=O)CC3(C)C)nn(C3CC4(C3)CN(C(=O)OC(C)(C)C)C4)c2C)c1Cl ; NC1COC1 ; CCN(C1COC1)C1CCN(c2nn(C3CC4(C3)CN(C(=O)OC(C)(C)C)C4)c(C)c2-c2c(Cl)c(C)cc3[nH]ncc23)C(C)(C)C1]

Tert-butyl 6-(4-(5-chloro-6-methyl-1H-indazol-4-yl)-3-(2,2-dimethyl-4-oxopiperidin-1-yl)-5-methyl-1H-pyrazol-1-yl)-2-azaspiro[3.3]heptane-2-carboxylate (Intermediate C111b, 1.50 g, 2.70 mmol) and oxetane-3-amine·HCl (0.58 g, 5.29 mmol) were dissolved in methanol (25 mL) and cooled to 0° C. under nitrogen atmosphere. Zinc chloride (1.08 g, 7.93 mmol) was added and the mixture stirred for 10 min. NaBH₃CN (0.50 g, 8.00 mmol) was then added portionwise and the RM was stirred at RT for 16 h. The RM was then cooled to 0° C., acetaldehyde (0.20 g, 5.30 mmol) was added and the reaction mixture was stirred at 50° C. for 1.5 h. After completion of the reaction, the RM was diluted with EtOAc, washed with water, brine, dried (Na₂SO₄), filtered and concentrated under vacuum. The crude residue was purified by chromatography on neutral alumina (eluent: 0 to 5.5% MeOH in CH₂Cl₂) to obtain the title product. UPLC-MS-7: Rt=4.55 min, 4.65 min, MS m/z [M+H]⁺ 652.4/653.4.

### Intermediate C113: Tert-butyl 6-(4-(5-chloro-6-methyl-1H-indazol-4-yl)-3-(4-(methyl(oxetan-3-yl)amino)-2,2-dimethylpiperidin-1-yl)-5-methyl-1H-pyrazol-1-yl)-2-azaspiro[3.3]heptane-2-carboxylate

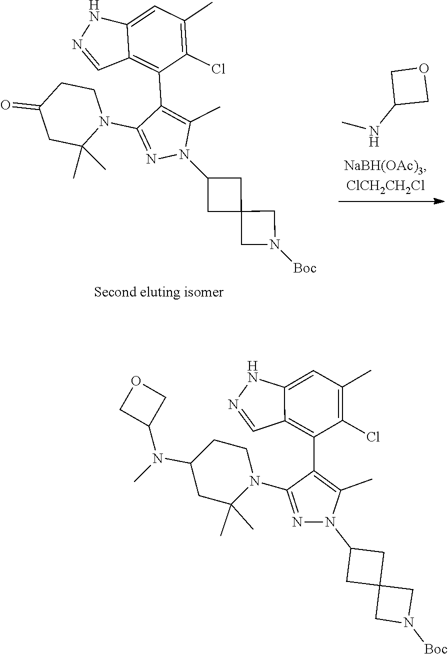

Tert-butyl 6-(4-(5-chloro-6-methyl-1H-indazol-4-yl)-3-(2,2-dimethyl-4-oxopiperidin-1-yl)-5-methyl-1H-pyrazol-1-yl)-2-azaspiro[3.3]heptane-2-carboxylate (Intermediate C111b, 384 mg, 0.68 mmol) and N-methyloxetan-3-amine (88.0 mg, 1.02 mmol) were dissolved in dichloroethane (5 mL) and cooled to 0° C. under nitrogen atmosphere. Then Sodium triacetoxyborohydride (421 mg, 2.03 mmol) was added and the reaction mixture was stirred at RT for 24 h. After completion of the reaction, the RM was poured into a sat. aq. NaHCO₃ solution and extracted with EtOAc (×2). The combined organic extracts were washed, dried (Na₂SO₄), filtered and concentrated under vacuum. The crude residue was purified by normal phase chromatography (eluent: 0 to 10% MeOH in CH₂Cl₂) to obtain the title product. UPLC-MS-7: Rt=1.01 min, 1.04 min, MS m/z [M+H]⁺ 638.4.

### Intermediates C114a and C114b: Tert-butyl 6-(3-(4-(hydroxymethyl)-2,2-dimethylpiperidin-1-yl)-5-methyl-1H-pyrazol-1-yl)-2-azaspiro[3.3]heptane-2-carboxylate

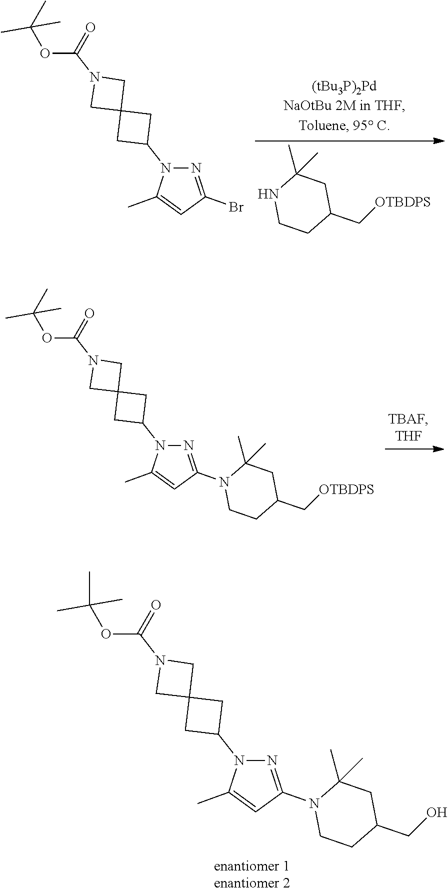

### Step 1: Tert-butyl 6-(3-(4-(((tert-butyldiphenylsilyl)oxy)methyl)-2,2-dimethylpiperidin-1-yl)-5-methyl-1H-pyrazol-1-yl)-2-azaspiro[3.3]heptane-2-carboxylate

A solution of tert-butyl 6-(3-bromo-5-methyl-1H-pyrazol-1-yl)-2-azaspiro[3.3]heptane-2-carboxylate (Intermediate C1, 2.00 g, 5.62 mmol) and 4-(((tert-butyldiphenylsilyl)oxy)methyl)-2,2-dimethylpiperidine (Intermediate A69, 2.78 g, 7.29 mmol) in 1,4-dioxane (30 mL) was degassed with nitrogen for 5 min and Pd(dba)₂ (0.32 g, 0.56 mmol) and 2-[bis(3,5-trifluoromethylphenylphosphino)-3,6-dimethoxy]-2′,6′-di-i-propoxy-1,1′-biphenyl (CAS [1810068-31-5], 0.47 g, 0.62 mmol) were added followed by NaOtBu (2.0 M in THF, 3.93 mL, 7.85 mmol). The reaction mixture was heated to 90° C. and stirred for 3 h in a sealed tube. The RM was filtered through a pad of celite and the filtrate was concentrated under vacuum. The crude residue was purified by column chromatography (neutral allumina, eluent: 100% CH₂Cl₂) to give the title product. UPLC-MS-5: Rt=2.75 min, MS m/z [M+H]⁺ 657.7.

### Step 2: Tert-butyl 6-(3-(4-(hydroxymethyl)-2,2-dimethylpiperidin-1-yl)-5-methyl-1H-pyrazol-1-yl)-2-azaspiro[3.3]heptane-2-carboxylate

To a solution of tert-butyl 6-(3-(4-(((tert-butyldiphenylsilyl)oxy)methyl)-2,2-dimethylpiperidin-1-yl)-5-methyl-1H-pyrazol-1-yl)-2-azaspiro[3.3]heptane-2-carboxylate (Step 1, 2.10 g, 3.19 mmol) in dry THF (20 mL) under nitrogen atmosphere at 0° C. was added TBAF (1M in THF, 6.40 mL, 6.40 mmol) and the reaction mixture was stirred at RT for 2 h. After completion of the reaction, the RM was concentrated under reduced pressure, quenched with a sat. aq. NaHCO₃ solution and extracted with EtOAc. The combined organic extracts were washed with water, brine, dried (Na₂SO₄), filtered and concentrated under vacuum. The crude residue purified by reverse phase chromatography (eluent: 40 to 50% CH₃CN in H₂O containing 0.025% NH₃) to give the title compound Intermediate C114-rac. The enantiomers were separated by chiral preparative HPLC (C-HPLC-25; mobile phase: [n-Hexane+0.1% DEA]/[MeOH/IPA 50/50]: 90/10); flow rate: 18 mL/min) to give the first eluting enantiomer of the title compound: Intermediate C114a; C-HPLC-26 (mobile phase: [n-Hexane+0.1% DEA]/[MeOH/IPA 50/50]: 90/10): Rt=15.3 min, UPLC-MS-5: Rt=1.40 min; MS m/z [M+H]⁺ 419.4 and the second eluting enantiomer of the title compound: Intermediate C114b: C-HPLC-26 (mobile phase: [n-Hexane+0.1% DEA]/[MeOH/IPA 50/50]: 90/10): Rt=18.4 min, UPLC-MS-5: Rt=1.40 min; MS m/z [M+H]⁺ 419.4.

### Intermediate C115: Tert-butyl (R)-6-(3-(4-(hydroxymethyl)-2,2-dimethylpiperidin-1-yl)-5-methyl-1H-pyrazol-1-yl)-2-azaspiro[3.3]heptane-2-carboxylate

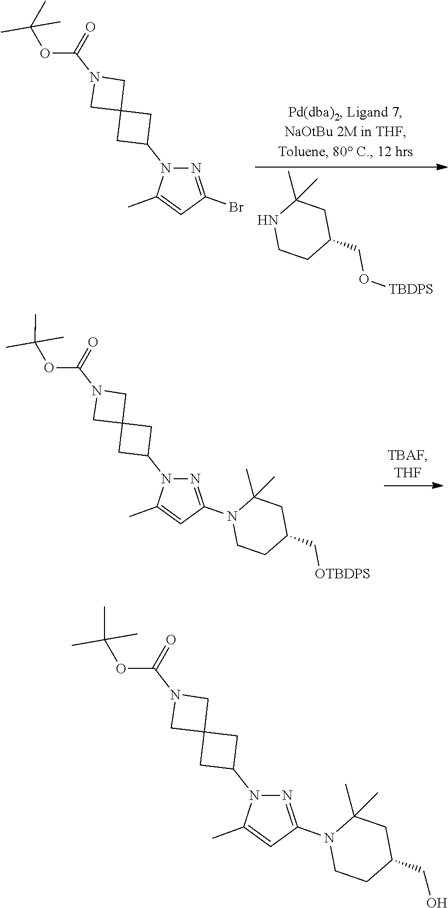

### Step 1: Tert-butyl (R)-6-(3-(4-(((tert-butyldiphenylsilyl)oxy)methyl)-2,2-dimethylpiperidin-1-yl)-5-methyl-1H-pyrazol-1-yl)-2-azaspiro[3.3]heptane-2-carboxylate

To a solution of (R)-4-(((tert-butyldiphenylsilyl)oxy)methyl)-2,2-dimethylpiperidine (Intermediate A85, 60 g, 157 mmol), tert-butyl 6-(3-bromo-5-methyl-1H-pyrazol-1-yl)-2-azaspiro[3.3]heptane-2-carboxylate (Intermediate C1, 53.3 g, 149 mmol), bis(3,5-bis(trifluoromethyl)phenyl)(2′,6′-bis(dimethylamino)-3,6-dimethoxybiphenyl-2-yl)phosphine (CAS [1810068-30-4], 6.00 g) in toluene (1.20 L) was added NaOtBu (2.0M in THF, 149 mL) and Pd(dba)₂ (8.61 g, 14.9 mmol). The reaction mixture was stirred at 80° C. for 12 h. The RM was quenched by addition of an aq. solution of NaHCO₃ (15.0%, 800 mL) and extracted with EtOAc (800 mL). The combined organic layers were washed three times with an aq. solution NaHCO₃ (15.0%, 500 mL), dried (Na₂SO₄), filtered and concentrated under reduced pressure. The crude residue was purified by normal phase column chromatography (eluent: Petroleum ether/Ethyl acetate I/O to 0/1) to give the title compound as a yellow oil. LCMS-19: Rt=0.99 min, MS m/z [M+H]⁺ 657.2.

### Step 2: Tert-butyl (R)-6-(3-(4-(hydroxymethyl)-2,2-dimethylpiperidin-1-yl)-5-methyl-1H-pyrazol-1-yl)-2-azaspiro[3.3]heptane-2-carboxylate

To a solution of tert-butyl (R)-6-(3-(4-(((tert-butyldiphenylsilyl)oxy)methyl)-2,2-dimethylpiperidin-1-yl)-5-methyl-1H-pyrazol-1-yl)-2-azaspiro[3.3]heptane-2-carboxylate (Step 1, 152 g, 231 mmol) in THF (1.52 L) was added TBAF (1 M, 462 mL, 462 mmol). The reaction mixture was stirred at 25° C. for 2 h. The RM was quenched by addition of an aq. Solution of NaHCO₃ (15.0%, 800 mL) and extracted with EtOAc (1000 mL). The combined organic layers were washed three times with an aq. Solution of NaHCO₃ (15.0%, 500 mL), dried (Na₂SO₄), filtered and concentrated under reduced pressure. The crude residue was purified by normal phase chromatography (eluent: Petroleum ether/Ethyl acetate=1/0 to 0/1) to give the title compound as a yellow foam. UPLC-MS-4: Rt=0.70 min, MS m/z [M+H]⁺ 419.6.

### Intermediate C116: Tert-butyl (R)-6-(3-(2,2-dimethyl-4-(morpholinomethyl)piperidin-1-yl)-5-methyl-1H-pyrazol-1-yl)-2-azaspiro[3.3]heptane-2-carboxylate

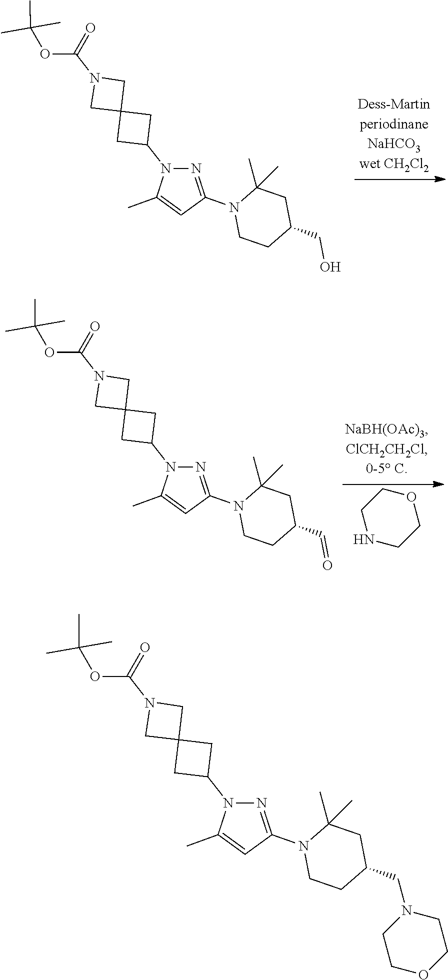 [SMILES: Cc1cc(N2CC[C@@H](CO)CC2(C)C)nn1C1CC2(C1)CN(C(=O)OC(C)(C)C)C2 ; Cc1cc(N2CC[C@@H](C=O)CC2(C)C)nn1C1CC2(C1)CN(C(=O)OC(C)(C)C)C2 ; C1COCCN1 ; Cc1cc(N2CC[C@@H](CN3CCOCC3)CC2(C)C)nn1C1CC2(C1)CN(C(=O)OC(C)(C)C)C2]

### Step 1: Tert-butyl (R)-6-(3-(4-formyl-2,2-dimethylpiperidin-1-yl)-5-methyl-1H-pyrazol-1-yl)-2-azaspiro[3.3]heptane-2-carboxylate

An ice-cooled mixture of tert-butyl (R)-6-(3-(4-(hydroxymethyl)-2,2-dimethylpiperidin-1-yl)-5-methyl-1H-pyrazol-1-yl)-2-azaspiro[3.3]heptane-2-carboxylate (Intermediate C115, 8.50 g, 20.3 mmol), NaHCO₃ (3.41 g, 40.6 mmol) and Dess-Martin periodinane (11.2 g, 26.4 mmol) was vigorously stirred in CH₂Cl₂ (196 mL) and wet CH₂Cl₂ (476 μL of water in 4 mL of CH₂Cl₂) was added. The reaction mixture was stirred at RT for 40 min, Dess-Martin periodinane (4.31 g, 10.15 mmol) was added again and the RM further stirred for 50 min to complete the reaction. The mixture was diluted with Et₂O (50 mL) and concentrated to a few mL of solvent. Et₂O (150 mL) was added and the mixture was filtered over a pad of celite and washed with Et₂O. The filtrate was poured into a 1/1 mixture of sat. aq. NaHCO₃/aq. Na₂S₂O₃ (10%), the layers were separated and the aqueous layer was back-extracted with Et₂O (×2). The combined organic layers were washed with a sat. aq. NaHCO₃ solution, dried (Na₂SO₄), filtered and concentrated under reduced pressure to give the title compound as a hygroscopic slightly brown foam which was used without purification in the next step. UPLC-MS-4: Rt=0.88 min; MS m/z [M+H]⁺ 417.5.

### Step 2: Tert-butyl (R)-6-(3-(2,2-dimethyl-4-(morpholinomethyl)piperidin-1-yl)-5-methyl-1H-pyrazol-1-yl)-2-azaspiro[3.3]heptane-2-carboxylate

A solution of tert-butyl (R)-6-(3-(4-formyl-2,2-dimethylpiperidin-1-yl)-5-methyl-1H-pyrazol-1-yl)-2-azaspiro[3.3]heptane-2-carboxylate (Step 1, 780 mg, 1.78 mmol) and morpholine (0.19 mL, 2.13 mmol) in dichloroethane (9 mL) was stirred under a nitrogen atmosphere at 0-5° C. for 10 min. Sodium triacetoxyborohydride (566 mg, 2.67 mmol) was added and the reaction mixture was stirred at 0-5° C. for 10 min. The RM was poured into a sat. aq. NaHCO₃ solution and extracted with CH₂Cl₂ (×3). The combined organic layers were dried (phase separator) and concentrated. The crude residue was purified by normal phase chromatography (eluent: MeOH in CH₂Cl₂ 0 to 8%) to give the title compound. UPLC-MS-4: Rt=0.62 min; MS m/z [M+H]⁺ 488.6.

### Intermediate C117: Tert-butyl (R)-6-(3-(4-(((N,4-dimethylpiperazine)-1-sulfonamido)methyl)-2,2-dimethylpiperidin-1-yl)-5-methyl-1H-pyrazol-1-yl)-2-azaspiro[3.3]heptane-2-carboxylate

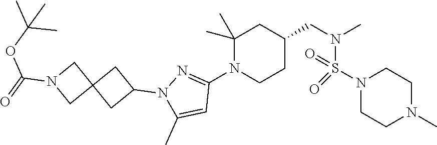 [SMILES: Cc1cc(N2CC[C@@H](CN(C)S(=O)(=O)N3CCN(C)CC3)CC2(C)C)nn1C1CC2(C1)CN(C(=O)OC(C)(C)C)C2]

### Step 1: Tert-butyl (R)-6-(3-(2,2-dimethyl-4-((methylamino)methyl)piperidin-1-yl)-5-methyl-1H-pyrazol-1-yl)-2-azaspiro[3.3]heptane-2-carboxylate

A solution of tert-butyl (R)-6-(3-(4-formyl-2,2-dimethylpiperidin-1-yl)-5-methyl-1H-pyrazol-1-yl)-2-azaspiro[3.3]heptane-2-carboxylate (described in the synthesis of Intermediate C116 (Step 1), 1.21 mg, 2.90 mmol) and methylamine (2M in MeOH, 7.24 mL, 14.5 mmol) in dichloroethane (14.5 mL) was stirred under a nitrogen atmosphere at 0-5° C. for 10 min. Sodium triacetoxyborohydride (921 mg, 4.34 mmol) was added and the reaction mixture was stirred at RT for 14 h. Methylamine (2M in MeOH, 7.24 mL, 14.5 mmol) was added again followed by sodium triacetoxyborohydride (921 mg, 4.34 mmol) and the RM was stirred at RT for 5 h (this operation was repeated again). The RM was then poured into a sat. aq. NaHCO₃ solution and extracted with CH₂Cl₂ (×3). The combined organic layers were dried (phase separator) and concentrated. The crude residue was used without purification in the next step. UPLC-MS-4: Rt=0.62 min; MS m/z [M+H]⁺ 432.6.

### Step 2: Tert-butyl (R)-6-(3-(4-(((N,4-dimethylpiperazine)-1-sulfonamido)methyl)-2,2-dimethylpiperidin-1-yl)-5-methyl-1H-pyrazol-1-yl)-2-azaspiro[3.3]heptane-2-carboxylate

A solution of tert-butyl (R)-6-(3-(2,2-dimethyl-4-((methylamino)methyl)piperidin-1-yl)-5-methyl-1H-pyrazol-1-yl)-2-azaspiro[3.3]heptane-2-carboxylate (Step 1, 1.25 g, 2.89 mmol), 4-methylpiperazine-1-sulfonyl chloride hydrochloride (0.74 g, 3.17 mmol) and DIPEA (1.51 mL, 8.66 mmol) in CH₂Cl₂ (14 mL) was stirred at RT for 5 h under nitrogen atmosphere. The reaction mixture was poured into an aq. sat. NaHCO₃ solution and extracted with CH₂Cl₂ (×2). The combined organic extracts were dried (phase separator) and concentrated in vacuo. The crude residue was purified by normal phase chromatography (eluent: MeOH in CH₂Cl₂ from 0 to 10%) to give the title compound as a beige foam. UPLC-MS-4: Rt=0.66 min; MS m/z [M+H]⁺ 594.7.

### Method-C118 for the Preparation of Intermediates C118a and C118b: Tert-butyl 6-(3-((R)-2,2-dimethyl-4-(((4aR*,7aS*)-4-methylhexahydropyrrolo[3,4-b][1,4]oxazin-6(2H)-yl)methyl)piperidin-1-yl)-5-methyl-1H-pyrazol-1-yl)-2-azaspiro[3.3]heptane-2-carboxylate isomer 1 and isomer 2

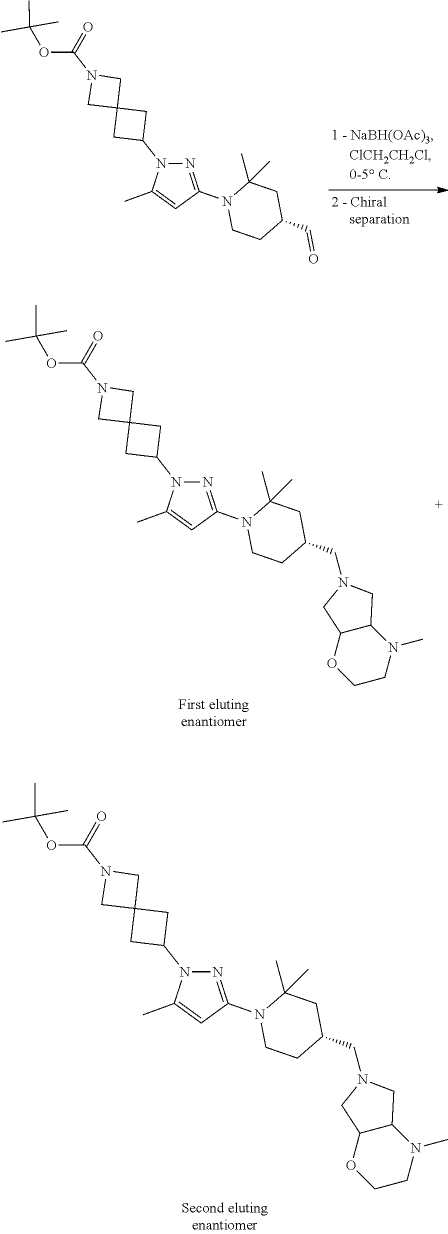

A solution of tert-butyl (R)-6-(3-(4-formyl-2,2-dimethylpiperidin-1-yl)-5-methyl-1H-pyrazol-1-yl)-2-azaspiro[3.3]heptane-2-carboxylate (described in the synthesis of Intermediate C116 (Step 1), 2.50 g, 6.00 mmol) and rac-(4aR*,7aS)-4-methyl-octahydropyrrolo[3,4-b]morpholine (0.85 g, 6.00 mmol) in dichloroethane (20 mL) was stirred under a nitrogen atmosphere at 0-5° C. for 10 min. Sodium triacetoxyborohydride (3.82 g, 18.0 mmol) was added and the reaction mixture was stirred at 0-5° C. for 1 h. The RM was poured into a sat. aq. NaHCO₃ solution and extracted with CH₂Cl₂ (×3). The combined organic layers were dried (phase separator) and concentrated. The crude residue was purified by normal phase chromatography (eluent: MeOH in CH₂Cl₂ 0 to 10%) to give the title compound. The enantiomers were separated by chiral preparative SFC (C-SFC-7; mobile phase: CO₂/[MeOH+0.025% NH₃]: 60/40) to give the first eluting isomer of the title compound: Intermediate C118a: C-SFC-8 (mobile phase: CO₂/[MeOH+0.025% NH₃]: 70/30): Rt=2.05 min, UPLC-MS-4: Rt=0.60 min; MS m/z [M+H]⁺ 543.4 and the second eluting isomer of the title compound: Intermediate C118b: C-SFC-8 (mobile phase: CO₂/[MeOH+0.025% NH₃]: 70/30): Rt=2.73 min; UPLC-MS-4: Rt=0.60 min; MS m/z [M+H]⁺ 543.4 min.

The following examples C119 to C120 were prepared using analogous methods to the method-C118 from commercially available reagents.

| | | Method, | |
|---|---|---|---|
| | | intermediates and | |
| | | chiral separation | |
| | | conditions used and | Characterization |
| Intermediate | Structure | order of elution | data |
| C119a/b | 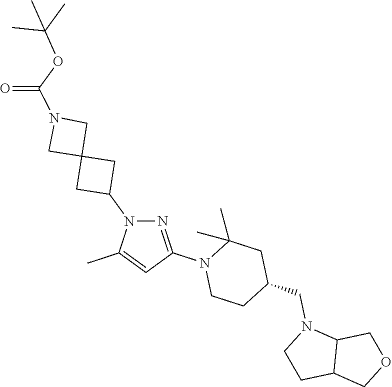 [SMILES: Cc1cc(N2CC[C@@H](CN3CCC4COCC43)CC2(C)C)nn1C1CC2(C1)CN(C(=O)OC(C)(C)C)C2] | Using Method-C118 from rac-(3aR,6aR)- hexahydro-1H-furo[3,4- B]pyrrole and C-SFC- 28 (mobile phase: CO₂/[IPA + 0.025% NH₃]: 75/25); Intermediate C119a = 1^{st} eluting isomer, Intermediate C119b = 2^{nd} eluting isomer | Intermediate C119a: UPLC- MS-4: Rt = 0.68 min; MS m/z [M + H]⁺ 514.4; C- SFC-29 (mobile phase: CO₂/[IPA + 0.05% Et₂NH]: from 95/5 to 60/40): Rt = 4.21 min, Intermediate C119b: UPLC- MS-4: Rt = 0.69 min; MS m/z [M + H]⁺ 514.4; C- SFC-29 (mobile phase: CO₂/[IPA + 0.05% Et₂NH]: from 95/5 to 60/40): Rt = 4.52 min. |
| | Tert-butyl 6-(3-((R)-4- | | |
| | (((3aR*,6aR*)-hexahydro-1H- | | |
| | furo[3,4-b]pyrrol-1-yl)methyl)- | | |
| | 2,2-dimethylpiperidin-1-yl)-5- | | |
| | methyl-1H-pyrazol-1-yl)-2- | | |
| | azaspiro[3.3]heptane-2- | | |
| | carboxylate | | |
| C120a/b | 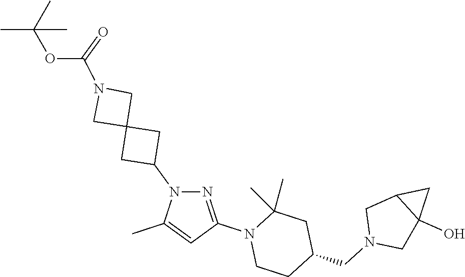 [SMILES: Cc1cc(N2CC[C@@H](CN3CC4CC4(O)C3)CC2(C)C)nn1C1CC2(C1)CN(C(=O)OC(C)(C)C)C2] | Using Method-C118 from 3- azabicyclo[3.1.0]hexan- 1-ol Hydrochloride and C-SFC-18 (mobile phase: CO₂/[IPA + 0.025% NH₃]: 60/40); Intermediate C120a = 1^{st} eluting isomer, Intermediate C120b = 2^{nd} eluting isomer | Intermediate C120a: UPLC- MS-4: Rt = 0.65 min; MS m/z [M + H]⁺ 500.5; C-SFC-19 (mobile phase: CO₂/[IPA + 0.012% NH₃]: 75/25): Rt = 2.78 min, Intermediate C120b: UPLC- MS-4: Rt = 0.65 min; MS m/z [M + H]⁺ 500.5; C-SFC- 19 (mobile phase: CO₂/[IPA + 0.012% NH₃]: 75/25): Rt = 3.56 min. |
| | Tert-butyl 6-(3-((4R)-4-((1- | | |
| | hydroxy-3- | | |
| | azabicyclo[3.1.0]hexan-3- | | |
| | yl)methyl)-2,2- | | |
| | dimethylpiperidin-1-yl)-5- | | |
| | methyl-1H-pyrazol-1-yl)-2- | | |
| | azaspiro[3.3]heptane-2- | | |
| | carboxylate | | |

### Intermediates 0121: Tert-butyl 6-(3-((R)-2,2-dimethyl-4-(((4aS*,7aR*)-4-(oxetan-3-yl)hexahydrofuro[3,4-b]pyrazin-1(2H)-yl)methyl) piperidin-1-yl)-5-methyl-1H-pyrazol-1-yl)-2-azaspiro[3.3]heptane-2-carboxylate Isomer 2

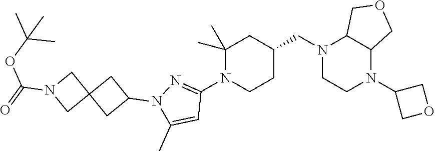 [SMILES: Cc1cc(N2CC[C@@H](CN3CCN(C4COC4)C4COCC43)CC2(C)C)nn1C1CC2(C1)CN(C(=O)OC(C)(C)C)C2]

### Step 1: rac-2-(trimethylsilyl)ethyl (4aS*,7aR*)-hexahydrofuro[3,4-b]pyrazine-1(2H)-carboxylate

To an ice-cooled stirred solution of (4aR*,7aS*)-octahydrofuro[3,4-b]pyrazine (500 mg, 3.71 mmol) in CH₂Cl₂ (15 mL) was added under argon DIPEA (5.18 mL, 29.6 mmol) and 1-[2-(trimethylsilyl)ethoxycarbonyl)pyrrolidin-2,5-dione (941 mg, 3.56 mmol) in CH₂Cl₂ (15 mL). The reaction mixture was stirred at RT for 17 h. The RM was poured into an aq. sat. NaHCO₃ solution and extracted with CH₂Cl₂ (×3). The combined organic layers were dried (phase separator), concentrated and the crude residue was purified by normal phase chromatography (eluent: MeOH in CH₂Cl₂ from 0 to 6%) to give the title product as a colorless oil. UPLC-MS-4: Rt=0.58 min; MS m/z [M+H]⁺ 273.3.

### Step 2: 2-(Trimethylsilyl)ethyl (4aR*,7aS*)-4-(((R)-1-(1-(2-(tert-butoxycarbonyl)-2-azaspiro[3.3]heptan-6-yl)-5-methyl-1H-pyrazol-3-yl)-2,2-dimethylpiperidin-4-yl)methyl)hexahydrofuro[3,4-b]pyrazine-1(2H)-carboxylate Isomer 1 and Isomer 2

To a solution of tert-butyl (R)-6-(3-(4-formyl-2,2-dimethylpiperidin-1-yl)-5-methyl-1H-pyrazol-1-yl)-2-azaspiro[3.3]heptane-2-carboxylate (described in the synthesis of Intermediate C116 (Step 1), 4.30 g, 9.81 mmol) in MeOH (60 mL) cooled at 0° C. was added under Ar rac-2-(trimethylsilyl)ethyl (4aS*,7aR*)-hexahydrofuro[3,4-b]pyrazine-1(2H)-carboxylate (Step 1, 2.85 g, 9.94 mmol) in MeOH (20 mL) and the reaction mixture was stirred at 0° C. for 30 min. Then, sodium triacetoxyborohydride (3.12 g, 14.7 mmol) was added and the RM was slowly allowed to warm to RT and stirred overnight. The RM was quenched with a sat. aq. NaHCO₃ solution and extracted with EtOAc (×3). The combined organic layer was dried (phase separator), evaporated and the crude residue was purified by normal phase chromatography (eluent: 0 to 80% EtOAc in heptane). The diastereoisomers were separated by chiral preparative SFC (C-SFC-4; mobile phase: CO₂/[IPA+0.025% NH₃]: 78/22) to give the first eluting isomer of the title compound: Isomer 1: C-SFC-3 (mobile phase: CO₂/[IPA+0.012% NH₃]: 80/20): Rt=1.10 min, UPLC-MS-4: Rt=1.44 min; MS m/z [M+H]⁺ 673.6 and the second eluting isomer of the title compound: Isomer 2: C-SFC-3 (mobile phase: CO₂/[IPA+0.012% NH₃]: 70/30): Rt=2.81 min; UPLC-MS-4: Rt=1.42 min; MS m/z [M+H]⁺ 673.3.

### Step 3: Tert-butyl 6-(3-((R)-4-(((4aS*,7aR*)-hexahydrofuro[3,4-b]pyrazin-1(2H)-yl)methyl)-2,2-dimethylpiperidin-1-yl)-5-methyl-1H-pyrazol-1-yl)-2-azaspiro[3.3]heptane-2-carboxylate Isomer 2

To a solution of 2-(trimethylsilyl)ethyl (4aR*,7aS)-4-(((R)-1-(1-(2-(tert-butoxycarbonyl)-2-azaspiro[3.3]heptan-6-yl)-5-methyl-1H-pyrazol-3-yl)-2,2-dimethylpiperidin-4-yl)methyl)hexahydrofuro[3,4-b]pyrazine-1(2H)-carboxylate Isomer 2 (Step 2, 1.73 g, 2.44 mmol) in THF (25 mL) was added TBAF (1M in THF, 7.00 mL, 7.00 mmol) and the reaction mixture was stirred at RT for 1.5 h. The RM was diluted with 10% aq. NH₄Cl solution and extracted with EtOAc (×2). The combined organic layer was washed with a sat. aq. NaHCO₃ solution, with brine, dried (phase separator) and evaporated. The crude residue was purified by normal phase chromatography (eluent: MeOH in CH₂Cl₂ from 0 to 15%) to give the title product as a white foam. UPLC-MS-4: Rt=0.58 min; MS m/z [M+H]⁺ 529.6.

### Step 4: Tert-butyl 6-(3-((R)-2,2-dimethyl-4-(((4aS*,7aR*-4-(oxetan-3-yl)hexahydrofuro[3,4-b]pyrazin-1(2H)-yl)methyl)piperidin-1-yl)-5-methyl-1H-pyrazol-1-yl)-2-azaspiro[3.3]heptane-2-carboxylate Isomer 2

To a solution of tert-butyl 6-(3-((R)-4-(((4aS*,7aR*)-hexahydrofuro[3,4-b]pyrazin-1(2H)-yl)methyl)-2,2-dimethylpiperidin-1-yl)-5-methyl-1H-pyrazol-1-yl)-2-azaspiro[3.3]heptane-2-carboxylate Isomer 2 (Step 3, 1.20 g, 2.15 mmol) in dichloroethane (25 mL), was added under Ar atmosphere oxetan-3-one (0.41 mL, 6.47 mmol) and the reaction mixture was stirred at RT for 3 min. Then, sodium triacetoxyborohydride (1.83 g, 8.62 mmol) was added and the RM was stirred at RT for 35 min. The RM was quenched by addition of a sat. aq. NaHCO₃ solution and extracted with EtOAc (×2). The combined organic layer was washed with brine, dried (phase separator) and evaporated. The crude residue was purified by normal phase chromatography (eluent: MeOH in CH₂Cl₂ from 0 to 10%). UPLC-MS-4: Rt=0.73 min; MS m/z [M+H]⁺ 585.7.

### Intermediate C122: Tert-butyl 6-(3-((R)-2,2-dimethyl-4-(((4aS*,7aR*)-4-(oxetan-3-yl)hexahydrofuro[3,4-b]pyrazin-1(2H)-yl)methyl)piperidin-1-yl)-5-methyl-1H-pyrazol-1-yl)-2-azaspiro[3.3]heptane-2-carboxylate Isomer 1

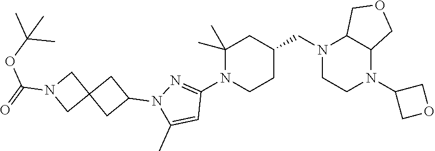 [SMILES: Cc1cc(N2CC[C@@H](CN3CCN(C4COC4)C4COCC43)CC2(C)C)nn1C1CC2(C1)CN(C(=O)OC(C)(C)C)C2]

The title compound was prepared by a method similar to that of tert-butyl 6-(3-((R)-2,2-dimethyl-4-(((4aS*,7aR*)-4-(oxetan-3-yl)hexahydrofuro[3,4-b]pyrazin-1(2H)-yl)methyl)piperidin-1-yl)-5-methyl-1H-pyrazol-1-yl)-2-azaspiro[3.3]heptane-2-carboxylate Isomer 2 (Intermediate C121) using 2-(trimethylsilyl)ethyl (4aR*,7aS*)-4-(((R)-1-(1-(2-(tert-butoxycarbonyl)-2-azaspiro[3.3]heptan-6-yl)-5-methyl-1H-pyrazol-3-yl)-2,2-dimethylpiperidin-4-yl)methyl)hexahydrofuro[3,4-b]pyrazine-1(2H)-carboxylate Isomer 1. UPLC-MS-4: Rt=0.74 min; MS m/z [M+H]⁺ 585.7.

### Method-C123 for the Preparation of Intermediates C123a and C123b: Tert-butyl 6-(3-((R)-2,2-dimethyl-4-(((4aR*,7aR*)-6-methyl-7-oxooctahydro-1H-pyrrolo[3,4-b]pyridin-1-yl)methyl)piperidin-1-yl)-4-iodo-5-methyl-1H-pyrazol-1-yl)-2-azaspiro[3.3]heptane-2-carboxylate isomer 1 and isomer 2

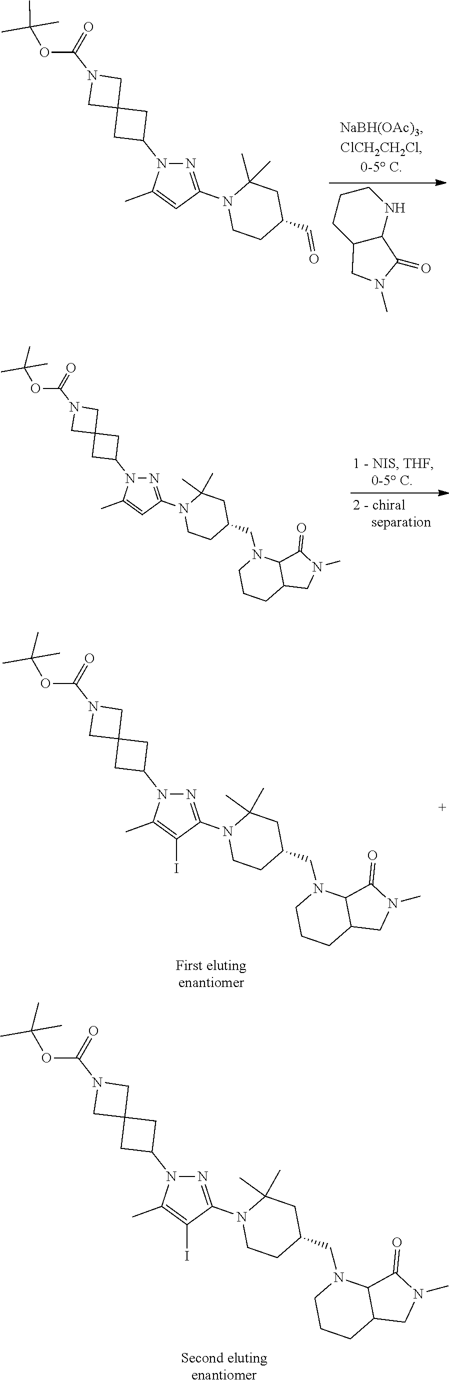

### Step 1: Tert-butyl 6-(3-((R)-2,2-dimethyl-4-(((4aR*,7aR*)-6-methyl-7-oxooctahydro-1H-pyrrolo[3,4-b]pyridin-1-yl)methyl)piperidin-1-yl)-5-methyl-1H-pyrazol-1-yl)-2-azaspiro[3.3]heptane-2-carboxylate

A solution of tert-butyl (R)-6-(3-(4-formyl-2,2-dimethylpiperidin-1-yl)-5-methyl-1H-pyrazol-1-yl)-2-azaspiro[3.3]heptane-2-carboxylate (described in the synthesis of Intermediate C116 (Step 1), 3.00 g, 7.20 mmol) and rac-(4aR*,7aS*-6-methyl-octahydro-1H-pyrrolo[3,4-B]pyridin-5-one (1.17 g, 7.56 mmol) in dichloroethane (20 mL) was stirred under a nitrogen atmosphere at 0-5° C. for 10 min. Sodium triacetoxyborohydride (4.58 g, 21.6 mmol) was added and the reaction mixture was stirred at 0-5° C. for 1 h. The RM was poured into a sat. aq. NaHCO₃ solution and extracted with CH₂Cl₂ (×3). The combined organic layers were washed with a sat. aq. NaHCO₃ solution, dried (phase separator) and concentrated. The crude residue was purified by normal phase chromatography (eluent: (EtOAc/EtOH 3/1) in c-Hexane 0 to 60%) to give the title compound. UPLC-MS-4: Rt=0.68 and 0.70 min; MS m/z [M+H]⁺ 555.7.

### Step 2: Tert-butyl 6-(3-((R)-2,2-dimethyl-4-(((4aR*,7aR*)-6-methyl-7-oxooctahydro-1H-pyrrolo[3,4-b]pyridin-1-yl)methyl)piperidin-1-yl)-4-iodo-5-methyl-1H-pyrazol-1-yl)-2-azaspiro[3.3]heptane-2-carboxylate isomer 1 and isomer 2

To an ice-cooled solution of tert-butyl 6-(3-((R)-2,2-dimethyl-4-(((4aR*,7aR*)-6-methyl-7-oxooctahydro-1H-pyrrolo[3,4-b]pyridin-1-yl)methyl)piperidin-1-yl)-5-methyl-1H-pyrazol-1-yl)-2-azaspiro[3.3]heptane-2-carboxylate (Step 1, 2.39 g, 4.31 mmol) in THF (20 mL) was added NIS (1.02 g, 4.52 mmol) and the mixture was stirred under N₂ atmosphere at 0° C. After completion (1 h), the reaction mixture was poured into 10% Na₂S₂O₃ aq. solution and extracted with CH₂Cl₂ (×2). The combined organic layers were washed with an aq. sat. NaHCO₃ solution, dried (phase separator) and concentrated. The crude residue was purified by normal phase chromatography (eluent: (EtOAc/EtOH 3/1) in c-Hexane 0 to 24%) to give the title product. The diastereoisomers were separated by chiral preparative SFC (C-SFC-5; mobile phase: CO₂/[MeOH+0.025% NH₃]: 80/20) to give the first eluting isomer of the title compound: Intermediate C123a: C-SFC-6 (mobile phase: CO₂/[MeOH+0.025% NH₃]: 80/20): Rt=2.31 min, UPLC-MS-4: Rt=1.08 min; MS m/z [M+H]⁺ 681.5 and the second eluting isomer of the title compound: Intermediate C123b: C-SFC-6 (mobile phase: CO₂/[MeOH+0.025% NH₃]: 70/30): Rt=1.08 min; UPLC-MS-4: Rt=2.74 min; MS m/z [M+H]⁺ 681.5 min.

Method-C123a: similar to Method-C123 except that Step 1 was performed in MeOH instead of Cl(CH₂)₂Cl.

The following examples C124 to C127 were prepared using analogous methods to Method-C123 from amines (in Step 1) described in the intermediates synthesis section A or commercially available.

| | | Method, intermediates | |
|---|---|---|---|
| | | and chiral separation | |
| | | conditions used to | Characterization |
| Intermediate | Structure | order of elution | data |
| C124a/b | 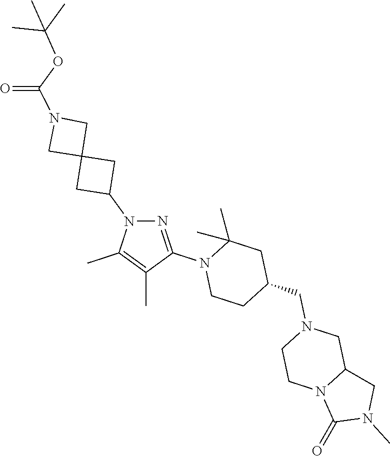 | Using Method-C123 from intermediate rac-2-methyl- octahydroimidazolidino[1,5- A]piperazin-3-one hydrochloride and C-SFC- 25 (mobile phase: CO₂/[IPA + 0.05% NH₃]: 70/30); Intermediate C124a = 1^{st} eluting isomer, Intermediate C124b = 2^{nd} eluting isomer | Intermediate C124a: UPLC-MS- 4: Rt = 1.06 min; MS m/z [M + H]⁺ 682.3; C-SFC-3 (mobile phase: CO₂/[IPA + 0.05% NH₃]: 70/30): Rt = 1.40 min, Intermediate C124b: UPLC-MS- 4: Rt = 1.06 min; MS m/z [M + H]⁺ 682.3; C-SFC-3 (mobile phase: CO₂/[IPA + 0.05% NH₃]: 70/30): Rt = 1.93 min. |
| | Tert-butyl 6-(3-((4R)-2,2- | | |
| | dimethyl-4-((2-methyl-3- | | |
| | oxohexahydroimidazo[1,5- | | |
| | a]pyrazin-7(1H)- | | |
| | yl)methyl)piperidin-1-yl)-4- | | |
| | iodo-5-methyl-1H-pyrazol-1- | | |
| | yl)-2-azaspiro[3.3]heptane- | | |
| | 2-carboxylate | | |
| C125a/b | 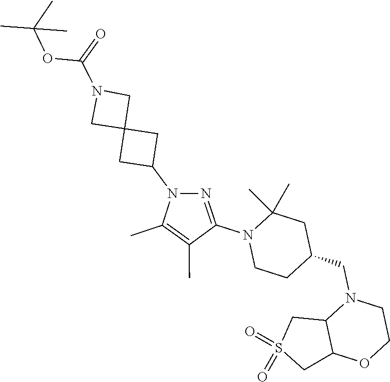 | Using Method-C123 from intermediate rac- (4aR*,7aS*)-hexahydro- 2H-6□6- thieno[3,4-b][1,4]oxazine- 6,6-dione hydrochloride and C-SFC-30 (mobile phase: CO₂/[IPA + 0.05% NH₃]: 65/35); Intermediate C125a = 1^{st} eluting isomer, Intermediate C125b = 2^{nd} eluting isomer | Intermediate C125a: UPLC-MS- 4: Rt = 1.33 min; MS m/z [M + H]⁺ 704.3; C-SFC-3 (mobile phase: CO₂/[IPA + 0.05% NH₃]: 70/30): Rt = 1.61 min, Intermediate C125b: UPLC-MS 4: Rt = 1.33 min; MS m/z [M + H]⁺ 704.3; C-SFC-3 (mobile phase: CO₂/[IPA + 0.05% NH₃]: 70/30): Rt = 2.93 min. |
| | Tert-butyl 6-(3-((R)-4- | | |
| | (((4aR*,7aS*)-6,6- | | |
| | dioxidohexahydro-4H- | | |
| | thieno[3,4-b][1,4]oxazin-4- | | |
| | yl)methyl)-2,2- | | |
| | dimethylpiperidin-1-yl)-4- | | |
| | iodo-5-methyl-1H-pyrazol-1- | | |
| | yl)-2-azaspiro[3.3]heptane- | | |
| | 2-carboxylate | | |
| C126a/b | 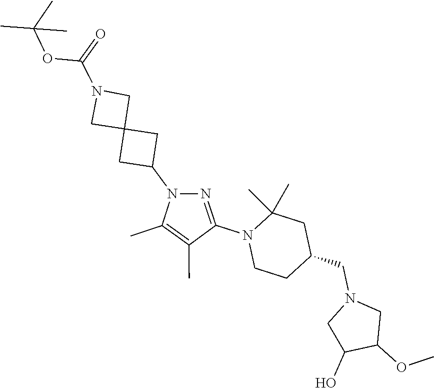 | Using Method-C123a from intermediate trans-4- methoxy-3-pyrrolidinol hydrochloride and C-SFC-4 (mobile phase: CO₂/[IPA + 0.05% Et₃N]: 70/30); Intermediate C126a = 1^{st} eluting isomer, Intermediate C126b = 2^{nd} eluting isomer | Intermediate C126a: UPLC-MS- 4: Rt = 0.94 min; MS m/z [M + H]⁺ 644.5; C-SFC-3 (mobile phase: CO₂/[IPA + 0.05% NH₃]: 80/20): Rt = 2.20 min, Intermediate C126b: UPLC-MS- 4: Rt = 0.97 min; MS m/z [M + H]⁺ 644.4; C-SFC-3 (mobile phase: CO₂/[IPA + 0.05% NH₃]: 80/20): Rt = 2.86 min. |
| | Tert-butyl 6-(3-((R)-4- | | |
| | (((3R*,4R*)-3-hydroxy-4- | | |
| | methoxypyrrolidin-1- | | |
| | yl)methyl)-2,2- | | |
| | dimethylpiperidin-1-yl)-4- | | |
| | iodo-5-methyl-1H-pyrazol-1- | | |
| | yl)-2-azaspiro[3.3]heptane-2- | | |
| | carboxylate | | |
| C127a/b | 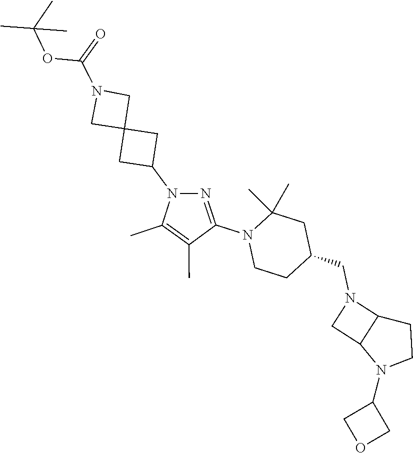 | Using Method-C123 from intermediate A5 and C- SFC-18 (mobile phase: CO₂/[EtOH + 0.025% NH₃]: 63/37); Intermediate C127a = 1^{st} eluting isomer, Intermediate C127b = 2^{nd} eluting isomer | Intermediate C127a: UPLC-MS- 4: Rt = 0.95 min; MS m/z [M + H]⁺ 681.5; C-SFC-19 (mobile phase: CO₂/[EtOH + 0.025% NH₃]: 63/37): Rt = 3.55 min, Intermediate C127b: UPLC-MS- 4: Rt = 0.93 min; MS m/z [M + H]⁺ 681.5; C-SFC-19 (mobile phase: CO₂/[EtOH + 0.025% NH₃]: 63/37): Rt = 4.21 min. |
| | Tert-butyl 6-(3-((R)-2,2- | | |
| | dimethyl-4-(((1S*,5S*)-2- | | |
| | (oxetan-3-yl)-2,6- | | |
| | diazabicyclo[3.2.0]heptan-6- | | |
| | yl)methyl)piperidin-1-yl)-4- | | |
| | iodo-5-methyl-1H-pyrazol-1- | | |
| | yl)-2-azaspiro[3.3]heptane- | | |
| | 2-carboxylate | | |

### Intermediates C128a and C128b: Tert-butyl 6-(3-(2,2-dimethyl-3-(morpholinomethyl)pyrrolidin-1-yl)-5-methyl-1H-pyrazol-1-yl)-2-azaspiro[3.3]heptane-2-carboxylate

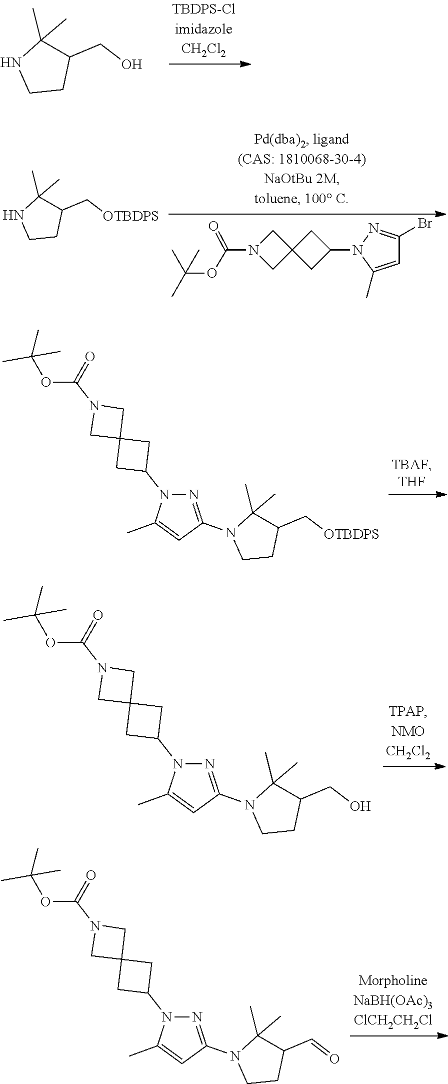

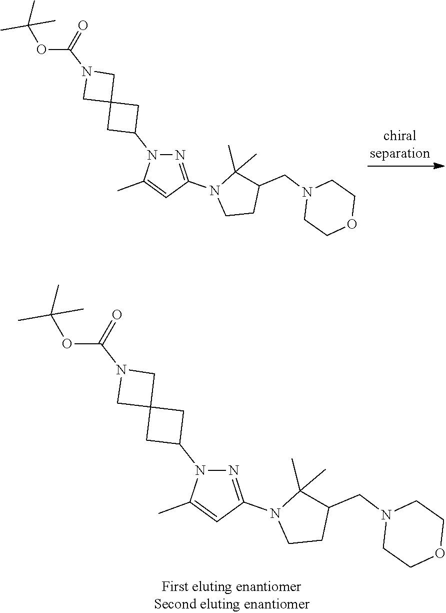 [SMILES: Cc1cc(N2CCC(CN3CCOCC3)C2(C)C)nn1C1CC2(C1)CN(C(=O)OC(C)(C)C)C2 ; Cc1cc(N2CCC(CN3CCOCC3)C2(C)C)nn1C1CC2(C1)CN(C(=O)OC(C)(C)C)C2]

### Step 1: 3-(((Tert-butyldiphenylsilyl)oxy)methyl)-2,2-dimethylpyrrolidine

To a solution of 2,2-dimethylpyrrolidin-3-yl)methanol (CAS [1538745-87-7], 2.20 g, 17.0 mmol) in CH₂Cl₂ (25 mL) at cooled to 0° C. was added imidazole (2.89 g, 42.6 mmol) followed by dropwise addition of TBDPS-chloride (7.00 g, 26.0 mmol) and the reaction mixture was stirred at RT for 16 h. A white solid precipitated and was filtered through a celite bed and washed with CH₂Cl₂. The filtrate was washed with water, brine, dried (Na₂SO₄), filtered and concentrated. The crude residue was purified by reverse phase chromatography (eluent: 35% CH₃CN in water containing 0.1% NH₃) to give the title as a pale yellow oil. UPLC-MS-17: Rt=4.68 min, MS m/z [M+H]⁺ 368.6.

### Step 2: Tert-butyl 6-(3-(3-(((tert-butyldiphenylsilyl)oxy)methyl)-2,2-dimethylpyrrolidin-1-yl)-5-methyl-1H-pyrazol-1-yl)-2-azaspiro[3.3]heptane-2-carboxylate

A solution of tert-butyl 6-(3-bromo-5-methyl-1H-pyrazol-1-yl)-2-azaspiro[3.3]heptane-2-carboxylate (Intermediate C1, 4.00 g, 11.0 mmol) and 3-(((tert-butyldiphenylsilyl)oxy)methyl)-2,2-dimethylpyrrolidine (Step 1, 4.30 g, 12.0 mmol) in toluene (40 mL) was degassed with nitrogen for 10 min while stirring. Bis(3,5-bis(trifluoromethyl)phenyl)(2′,6′-bis(dimethylamino)-3,6-dimethoxybiphenyl-2-yl)phosphine (CAS: [1810068-30-4], 0.67 g, 0.90 mmol) and Pd(dba)₂ (0.38 g, 0.67 mmol) were added followed by NaOtBu (2M in THF, 16.9 mL, 33.7 mmol) and the reaction mixture was stirred at 90° C. for 3 h. After completion of the reaction, the RM was diluted with EtOAc, filtered through a celite bed and washed with EtOAc. The filtrate was concentrated and the crude residue was purified by normal phase chromatography on neutral alumina (eluent: 3 to 7% EtOAc in hexane) to give the title compound. UPLC-MS-6: Rt=3.44 min, MS m/z [M+H]⁺ 643.8.

### Step 3: Tert-butyl 6-(3-(3-(hydroxymethyl)-2,2-dimethylpyrrolidin-1-yl)-5-methyl-1H-pyrazol-1-yl)-2-azaspiro[3.3]heptane-2-carboxylate

To a solution of tert-butyl 6-(3-(3-(((tert-butyldiphenylsilyl)oxy)methyl)-2,2-dimethylpyrrolidin-1-yl)-5-methyl-1H-pyrazol-1-yl)-2-azaspiro[3.3]heptane-2-carboxylate (Step 2, 2.80 g, 4.40 mmol) in THF (30 mL) at 0° C. was added dropwise TBAF (1M in THF, 8.70 mL, 8.70 mmol) and the reaction mixture was stirred at RT for 8 h. After completion of the reaction, the RM was poured in ice-cold water and extracted with EtOAc (×2). The combined organic extracts were washed with water, brine, dried (Na₂SO₄), filtered and concentrated under vacuum. The crude residue was purified by reverse phase chromatography (eluent: CH₃CN in water containing 0.1% of HCOOH from 40 to 60%) to give the title compound. UPLC-MS-9: Rt=1.14 min, MS m/z [M+H]⁺ 405.8.

### Step 4: Tert-butyl 6-(3-(3-formyl-2,2-dimethylpyrrolidin-1-yl)-5-methyl-1H-pyrazol-1-yl)-2-azaspiro[3.3]heptane-2-carboxylate

To a solution of tert-butyl 6-(3-(3-(hydroxymethyl)-2,2-dimethylpyrrolidin-1-yl)-5-methyl-1H-pyrazol-1-yl)-2-azaspiro[3.3]heptane-2-carboxylate (Step 3, 1.35 g, 3.34 mmol) in CH₂Cl₂ (15 mL) was added molecular sieves followed by NMO (0.97 g, 8.34 mmol). The reaction mixture was cooled to 0° C., TPAP (0.12 g, 0.33 mmol) was added and reaction mixture was allowed to reach RT and stirred at RT for 4 h. The RM was filtered through a pad of celite and washed with excess of CH₂Cl₂. The filtrate was washed with cold water, brine, dried (Na₂SO₄), filtered and concentrated under vacuum to give the title compound which was used without purification in the next step. UPLC-MS-9: Rt=1.41 min, MS m/z [M+H]⁺ 403.5.

### Step 5: Tert-butyl 6-(3-(2,2-dimethyl-3-(morpholinomethyl)pyrrolidin-1-yl)-5-methyl-1H-pyrazol-1-yl)-2-azaspiro[3.3]heptane-2-carboxylate

A solution of tert-butyl 6-(3-(3-formyl-2,2-dimethylpyrrolidin-1-yl)-5-methyl-1H-pyrazol-1-yl)-2-azaspiro[3.3]heptane-2-carboxylate (Step 4, 1.15 g, 2.86 mmol) and morpholine (0.37 g, 4.28 mmol) in dichloroethane (15 mL) was allowed to stirret at RT for 2 h. The reaction mixture was cooled to 0° C. and NaBH(OAc)₃ (1.51 g, 7.14 mmol) was added. The RM was then allowed to reached RT and was stirred for 16 h. After completion of the reaction, the RM was poured into ice-cold water, neutralized with NaHCO₃ and extracted with EtOAc. The combined organic extracts were was washed with water, brine, dried (Na₂SO₄), filtered and concentrated under vacuum. The crude residue was purified by reverse phase chromatography (eluent: CH₃CN in water+0.1% formic acid from 55 to 60%) to give the title compound. The enantiomers were separated by chiral preparative SFC C-SFC-52; mobile phase: CO₂/[IPA+0.1% Et₂NH]: 82/18) to give the first eluting isomer of the title compound: Intermediate C128a: C-SFC-53 (mobile phase: CO₂/[IPA+0.1% Et₂NH]: 75/25): Rt=5.77 min, UPLC-MS-9: Rt=1.10 min, MS m/z [M+H]⁺ 475.0 and the second eluting isomer of the title compound: Intermediate C128b: C-SFC-53 (mobile phase: CO₂/[IPA+0.1% Et₂NH]: 75/25): Rt=6.54 min; UPLC-MS-9: Rt=1.10 min, MS m/z [M+H]⁺ 475.0.

### Intermediates C129a and C129b: Tert-butyl 6-(3-(4-hydroxy-2,2-dimethyl-4-(morpholinomethyl)piperidin-1-yl)-5-methyl-1H-pyrazol-1-yl)-2-azaspiro[3.3]heptane-2-carboxylate Isomer 1 and isomer 2

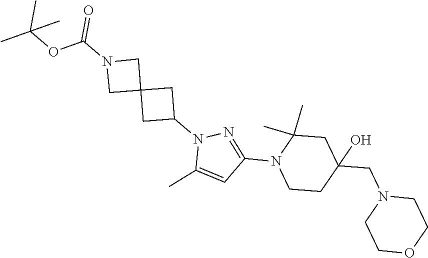 [SMILES: Cc1cc(N2CCC(O)(CN3CCOCC3)CC2(C)C)nn1C1CC2(C1)CN(C(=O)OC(C)(C)C)C2]

### Step 1: Tert-butyl 6-(3-(5,5-dimethyl-1-oxa-6-azaspiro[2.5]octan-6-yl)-5-methyl-1H-pyrazol-1-yl)-2-azaspiro[3.3]heptane-2-carboxylate

To a suspension of sodium hydride (60% in mineral oil, 862 mg, 21.5 mmol) in DMSO (60 mL) under Ar was added at 0° C. trimethylsulfoxonium iodide (4.84 g, 21.5 mmol). The reaction mixture was allowed to reach RT and stirred for 1 h. The reaction mixture was cooled to 0° C. and tert-butyl 6-(3-(2,2-dimethyl-4-oxopiperidin-1-yl)-5-methyl-1H-pyrazol-1-yl)-2-azaspiro[3.3]heptane-2-carboxylate (Intermediate C74, 5.90 g, 14.4 mmol) was added. The RM was allowed to reach RT and stirred for 3 h. The RM was quenched by addition of a sat. aq. NaHCO₃ solution and extracted with EtOAc (×2). The combined organic extracts were washed with brine, dried (phase separator) and evaporated. The crude residue was purified by normal phase chromatography (eluent: EtOAc in n-heptane from 0 to 100%) to give the title compound as a gum. UPLC-MS-4: Rt=1.06 min; MS m/z [M+H]⁺ 417.5.

### Step 2: Tert-butyl 6-(3-(4-hydroxy-2,2-dimethyl-4-(morpholinomethyl)piperidin-1-yl)-5-methyl-1H-pyrazol-1-yl)-2-azaspiro[3.3]heptane-2-carboxylate Isomer 1 and isomer 2

To a solution of tert-butyl 6-(3-(5,5-dimethyl-1-oxa-6-azaspiro[2.5]octan-6-yl)-5-methyl-1H-pyrazol-1-yl)-2-azaspiro[3.3]heptane-2-carboxylate (Step 1, 1.03 g, 2.22 mmol) in dimethylacetamide (10 mL) was added morpholine (0.45 mL, 5.10 mmol) and the reaction mixture was heated at 130° C. under microwave irradiations for 2 h. Morpholine (0.45 mL, 5.10 mmol) was added again and the mixture was further heated at 130° C. for 6 h. The RM was diluted with a sat. aq. NaHCO₃ solution and extracted with EtOAc (×2). The combined organic extracts were washed with brine, dried (phase separator) and evaporated. The crude residue was purified by normal phase chromatography (eluent: MeOH in CH₂Cl₂ from 0 to 4%) to give the title compound as a racemic mixture. The enantiomers were separated by chiral SFC (C-SFC-7; mobile phase: CO₂/[MeOH+0.025% NH₃]: 75/35) to give the first eluting enantiomer of the title compound: Intermediate C129a; C-SFC-8 (mobile phase: CO₂/[MeOH+0.025% NH₃]: 75/35): Rt=1.62 min, UPLC-MS-4: Rt=0.68 min; MS m/z [M+H]⁺ 504.6 and the second eluting enantiomer of the title compound: Intermediate C129b: C-SFC-8 (mobile phase: CO₂/[MeOH+0.025% NH₃]: 75/35): Rt=2.32 min, UPLC-MS-4: Rt=0.70 min; MS m/z [M+H]⁺ 504.5.

### Intermediates C130a, C130b, C130c and C130d: Tert-butyl 6-(3-(2-(methoxymethyl)-2-methyl-4-(morpholinomethyl)piperidin-1-yl)-5-methyl-1H-pyrazol-1-yl)-2-azaspiro[3.3]heptane-2-carboxylate Isomer-1.1, 1.2, 2.1, 2.2

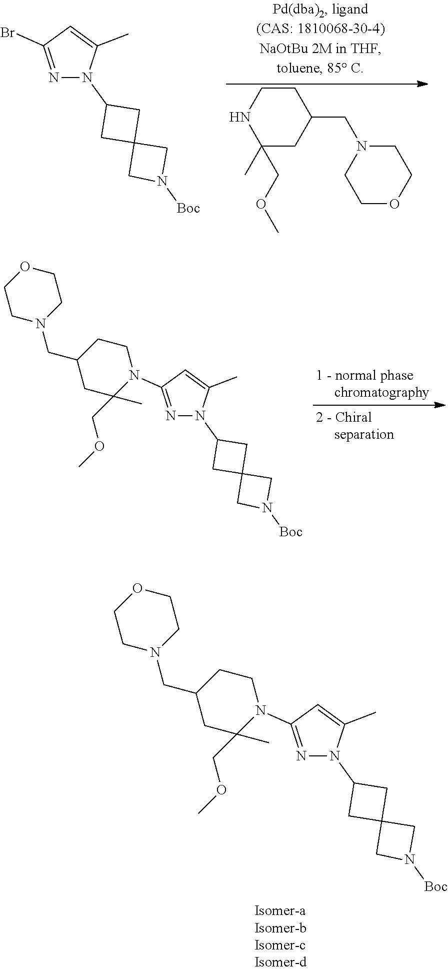 [SMILES: Cc1cc(Br)nn1C1CC2(C1)CN(C(=O)OC(C)(C)C)C2 ; COCC1(C)CC(CN2CCOCC2)CCN1 ; COCC1(C)CC(CN2CCOCC2)CCN1c1cc(C)n(C2CC3(C2)CN(C(=O)OC(C)(C)C)C3)n1 ; COCC1(C)CC(CN2CCOCC2)CCN1c1cc(C)n(C2CC3(C2)CN(C(=O)OC(C)(C)C)C3)n1]

A solution of tert-butyl 6-(3-bromo-5-methyl-1H-pyrazol-1-yl)-2-azaspiro[3.3]heptane-2-carboxylate (Intermediate C1, 0.90 g, 2.53 mmol) and 4-((2-(methoxymethyl)-2-methylpiperidin-4-yl)methyl)morpholine (Intermediate A86, 0.67 g, 2.78 mmol) in toluene (10 mL) was degassed with nitrogen for 10 min. Bis(3,5-bis(trifluoromethyl)phenyl)(2′,6′-bis(dimethylamino)-3,6-dimethoxybiphenyl-2-yl)phosphine (CAS [1810068-30-4], 0.15 g, 0.20 mmol) and Pd(dba)₂ (0.09 g, 0.15 mmol) were added followed by NaOtBu (2M in THF, 5.06 mL, 10.1 mmol) and the reaction mixture was stirred at 85° C. for 8 h. After completion of the reaction, the RM was diluted with EtOAc, filtered through a pad of celite and washed with EtOAc. The filtrate was concentrated under vacuum and the crude residue was purified by normal phase chromatography on neutral alumina (eluent: 25 to 30% EtOAc in hexane) to give the first eluting isomer of the title compound: Isomer-1 as a racemate: UPLC-MS-18 (mobile phase: A: 10 mM ammonium acetate in water/B: CH₃CN, gradient: 70/30 at 0.01 min, 50/50 at 10 min, 30/70 at 22 min, 0/100 at 26 min): Rt=15.80 min and the second eluting isomer of the title compound isomer-2 as a racemate: UPLC-MS-18 (mobile phase: A: 10 mM ammonium acetate in water/B: CH₃CN, gradient: 70/30 at 0.01 min, 50/50 at 10 min, 30/70 at 22 min, 0/100 at 26 min): Rt=15.40 min.

Tert-butyl 6-(3-(2-(methoxymethyl)-2-methyl-4-(morpholinomethyl)piperidin-1-yl)-5-methyl-1H-pyrazol-1-yl)-2-azaspiro[3.3]heptane-2-carboxylate Isomer-1 was further purified by preparative chiral HPLC (C-HPLC-28 (mobile Phase: MeOH containing 0.1% DEA) to give the first eluting enantiomer: Intermediate C130a: UPLC-MS-16: Rt=3.98 min, MS m/z [M+H]⁺ 518.4; C-HPLC-29 (mobile Phase: 100% MeOH), Rt=6.05 min and the second eluting enantiomer: Intermediate C130b: UPLC-MS-16: Rt=3.98 min, MS m/z [M+H]⁺ 518.4; C-HPLC-29 (mobile Phase: 100% MeOH), Rt=7.24 min.

Tert-butyl 6-(3-(2-(methoxymethyl)-2-methyl-4-(morpholinomethyl)piperidin-1-yl)-5-methyl-1H-pyrazol-1-yl)-2-azaspiro[3.3]heptane-2-carboxylate Isomer-2 was further purified by preparative chiral HPLC (C-HPLC-25 (mobile phase: [n-Hexane+0.1% DEA]/IPA:MeOH (50:50), isocratic 82/18), flow rate: 22 mL/min) to give the first eluting enantiomer: Intermediate C130c: UPLC-MS-9: Rt=1.07 min, MS m/z [M+H]⁺ 518.4; C-SFC-48 (mobile phase: CO₂/[MeOH+0.1% DEA], gradient: 5 to 50%), Rt=4.97 min and the second eluting enantiomer: Intermediate C130d: UPLC-MS-9: Rt=1.07 min, MS m/z [M+H]⁺ 518.4; C-SFC-48 (mobile phase: CO₂/[MeOH+0.1% DEA], gradient: 5 to 50%), Rt=5.23 min.

### Intermediates C131a, C131 b, C131c and C131d: Tert-butyl 6-(3-(2-ethyl-2-methyl-4-(morpholinomethyl)piperidin-1-yl)-5-methyl-1H-pyrazol-1-yl)-2-azaspiro[3.3]heptane-2-carboxylate Isomer-1.1, 1.2, 2.1, 2.2

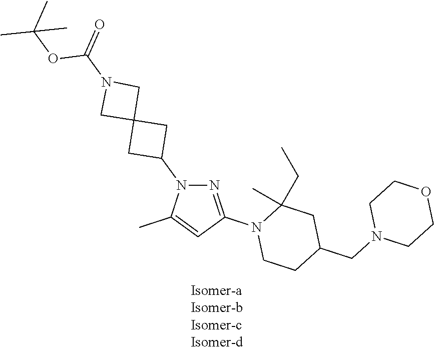 [SMILES: CCC1(C)CC(CN2CCOCC2)CCN1c1cc(C)n(C2CC3(C2)CN(C(=O)OC(C)(C)C)C3)n1]

The title compound was prepared by a method similar to that of tert-butyl 6-(3-(2-(methoxymethyl)-2-methyl-4-(morpholinomethyl)piperidin-1-yl)-5-methyl-1H-pyrazol-1-yl)-2-azaspiro[3.3]heptane-2-carboxylate (Intermediate C130) using 4-((2-ethyl-2-methylpiperidin-4-yl)methyl)morpholine (Intermediate A87) instead of 4-((2-(methoxymethyl)-2-methylpiperidin-4-yl)methyl)morpholine (Intermediate A86). The crude residue was purified by normal phase chromatography on neutral alumina (eluent: 50 to 80% EtOAc in hexane) to give the title compound as a mixture of 4 diastereosiomers. The diastereoisomers were separated by reverse phase preparative HPLC (RP-HPLC-5 (mobile phase: A: [5 mM ammonium bicarbonate+0.1% NH₃ in Water]/B: CH₃CN, gradient: 50 to 55% B in 34 min, 55 to 100% B in 2 min, 100 to 50% B in 6 min) to give the first eluting Isomer-1 as a racemate of the title compound: Isomer-1: UPLC-MS-16: Rt=4.40 min, MS m/z [M+H]⁺ 502.4 and the second eluting isomer of the title compound isomer-2 as a racemate: UPLC-MS-16: Rt=4.55 min, MS m/z [M+H]⁺ 502.4.

Tert-butyl 6-(3-(2-ethyl-2-methyl-4-(morpholinomethyl)piperidin-1-yl)-5-methyl-1H-pyrazol-1-yl)-2-azaspiro[3.3]heptane-2-carboxylate Isomer-1 was further purified by

preparative chiral HPLC (C-HPLC-28 (mobile phase: [MeOH+0.1% DEA]/CH₃CN 97/3; flow rate: 15 mL/min) to give the first eluting enantiomer: Intermediate C131a: UPLC-MS-9: Rt=1.07 min, MS m/z [M+H]⁺ 502.8; C-SFC-48 (mobile phase: CO₂/[MeOH+0.1% DEA], gradient: 5 to 50%), Rt=4.97 min and the second eluting enantiomer: Intermediate C131b: UPLC-MS-9: Rt=1.09 min, MS m/z [M+H]⁺ 502.8; C-SFC-48 (mobile phase: CO₂/[MeOH+0.1% DEA], gradient: 5 to 50%), Rt=5.23 min.

Tert-butyl 6-(3-(2-ethyl-2-methyl-4-(morpholinomethyl)piperidin-1-yl)-5-methyl-1H-pyrazol-1-yl)-2-azaspiro[3.3]heptane-2-carboxylate Isomer-2 was further purified by preparative chiral HPLC (C-SFC-52 (mobile phase: CO₂/[MeOH+0.1% DEA: CH₃CN (50:50)], isocratic: 85/15) to give the first eluting enantiomer: Intermediate C131c: UPLC-MS-9: Rt=1.08 min, MS m/z [M+H]⁺ 502.8; C-SFC-48 (mobile phase: CO₂/[MeOH+0.1% DEA], gradient: 5 to 50%), Rt=5.00 min and the second eluting enantiomer: Intermediate C131d: UPLC-MS-9: Rt=1.09 min, MS m/z [M+H]⁺ 502.8, C-SFC-48 (mobile phase: CO₂/[MeOH+0.1% DEA], gradient: 5 to 50%), Rt=5.22 min.

### Intermediate D1: 5-Chloro-6-methyl-1-(tetrahydro-2H-pyran-2-yl)-4-(4,4,5,5-tetramethyl-1,3,2-dioxaborolan-2-yl)-1H-indazole

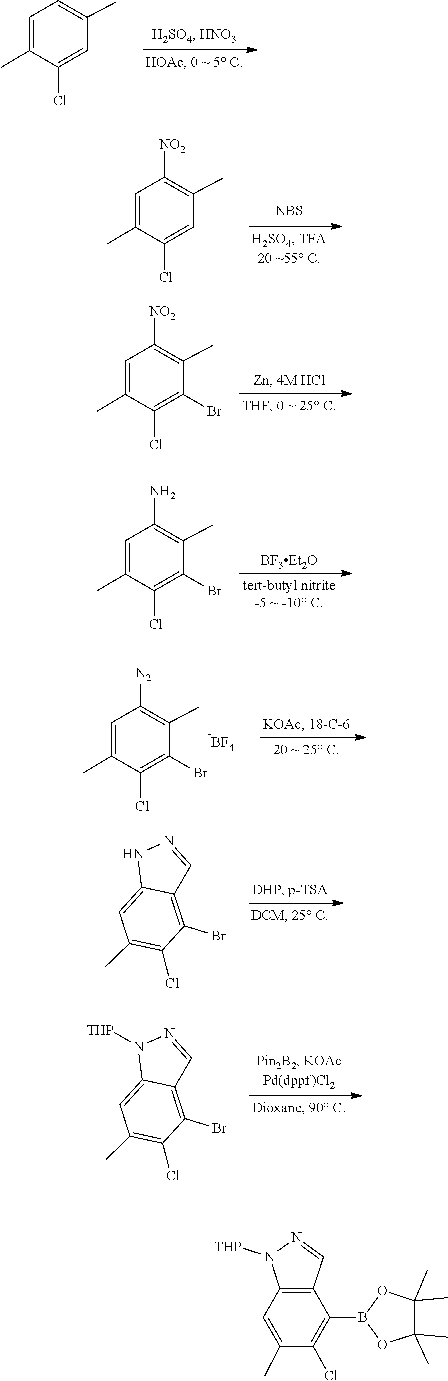

### Step 1: 1-Chloro-2,5-dimethyl-4-nitrobenzene

To an ice-cooled solution of 2-chloro-1,4-dimethylbenzene (3.40 kg, 24.2 mol) in AcOH (20.0 L) was added H₂SO₄ (4.74 kg, 48.4. mol, 2.58 L) followed by a dropwise addition (dropping funnel) of a cold solution of HNO₃ (3.41 kg, 36.3 mol, 2.44 L, 67.0% purity) in H₂SO₄ (19.0 kg, 193. mol, 10.3 L). The reaction mixture was then allowed to stir at 0-5° C. for 0.5 h. The reaction mixture was poured slowly into crushed ice (35.0 L) and the yellow solid precipitated out. The suspension was filtered and the cake was washed with water (5.00 L×5) to give a yellow solid which was suspended in MTBE (2.00 L) for 1 h, filtered, and dried to give the title compound as a yellow solid. ¹H NMR (400 MHz, CDCl₃) δ 7.90 (s, 1H), 7.34 (s, 1H), 2.57 (s, 3H), 2.42 (s, 3H).

### Step 2: 3-Bromo-2-chloro-1,4-dimethyl-5-nitrobenzene

To a cooled solution of 1-chloro-2,5-dimethyl-4-nitrobenzene (Step 1, 2.00 kg, 10.8 mol) in TFA (10.5 L) was slowly added concentrated H₂SO₄ (4.23 kg, 43.1 mol, 2.30 L) and the reaction mixture was stirred at 20° C. NBS (1.92 kg, 10.8 mol) was added in small portions and the reaction mixture was heated at 55° C. for 2 h. The reaction mixture was cooled to 25° C., then poured into crushed ice solution to obtain a pale white precipitate which was filtered through vacuum, washed with cold water and dried under vacuum to give the title compound (yellow solid) which was used without further purification in the next step. ¹H NMR (400 MHz, CDCl₃) δ 7.65 (s, 1H), 2.60 (s, 3H), 2.49 (s, 3H).

### Step 3: 3-Bromo-4-chloro-2,5-dimethylaniline

To a ice-cooled solution of 3-bromo-2-chloro-1,4-dimethyl-5-nitrobenzene (Step 2, 2.75 kg, 10.4 mol) in THF (27.5 L) was added HCl (4M, 15.6 L) then Zinc (2.72 kg, 41.6 mol) in small portions. The reaction mixture was allowed to stir at 25° C. for 2 h. The reaction mixture was basified by addition of a sat. aq. NaHCO₃ solution (until pH=8). The mixture was diluted with EtOAc (2.50 L) and stirred vigorously for 10 min and then filtered through a pad of celite. The organic layer was separated and the aqueous layer was re-extracted with EtOAc (3.00 L×4). The combined organic layers were washed with brine (10.0 L), dried (Na₂SO₄), filtered and concentrated under vacuum to give the title compound as a yellow solid which was used without further purification in the next step. ¹H NMR (400 MHz, DMSO-d₆) δ 6.59 (s, 1H), 5.23 (s, 2H), 2.22 (s, 3H), 2.18 (s, 3H).

### Step 4: 3-Bromo-4-chloro-2,5-dimethylbenzenediazonium tetrafluoroborate

BF₃·Et₂O (2.00 kg, 14.1 mol, 1.74 L) was dissolved in CH₂Cl₂ (20.0 L) and cooled to −5 to −10° C. under nitrogen atmosphere. A solution of 3-bromo-4-chloro-2,5-dimethylaniline (Step 3, 2.20 kg, 9.38 mol) in CH₂Cl₂ (5.00 L) was added to above reaction mixture and stirred for 0.5 h. Tert-butyl nitrite (1.16 kg, 11.3 mol, 1.34 L) was added dropwise and the reaction mixture was stirred at the same temperature for 1.5 h. TLC (petroleum ether:EtOAc=5:1) showed consumption of the starting material (R_{f}=0.45). MTBE (3.00 L) was added to the reaction mixture to give a yellow precipitate, which was filtered through vacuum and washed with cold MTBE (1.50 L×2) to give the title compound as a yellow solid which was used without further purification in the next step.

### Step 5: Intermediate D2: 4-Bromo-5-chloro-6-methyl-1H-indazole

To 18-Crown-6 ether (744 g, 2.82 mol) in chloroform (20.0 L) was added KOAc (1.29 kg, 13.2 mol) and the reaction mixture was cooled to 20° C. Then 3-bromo-4-chloro-2,5-dimethylbenzenediazonium tetrafluoroborate (Step 4, 3.13 kg, 9.39 mol) was added slowly. The reaction mixture was then allowed to stir at 25° C. for 5 h. After completion of the reaction, the reaction mixture was poured into ice cold water (10.0 L) and the aqueous layer was extracted with CH₂Cl₂ (5.00 L×3). The combined organic layers were washed with a sat. aq. NaHCO₃ solution (5.00 L), brine (5.00 L), dried (Na₂SO₄), filtered and concentrated under vacuum to give the title compound as a yellow solid. ¹H NMR (600 MHz, CDCl₃) δ 10.42 (br s, 1H), 8.04 (s, 1H), 7.35 (s, 1H), 2.58 (s, 3H). UPLC-MS-1a: Rt=1.02 min; MS m/z [M+H]⁺ 243/245/247.

### Step 6: Intermediate D3: 4-Bromo-5-chloro-6-methyl-1-(tetrahydro-2H-pyran-2-yl)-1H-indazole

To a solution of PTSA (89.8 g, 521 mmol) and 4-bromo-5-chloro-6-methyl-1H-indazole (Step 5, 1.28 kg, 5.21 mol) in CH₂Cl₂ (12.0 L) was added DHP (658 g, 7.82 mol, 715 mL) dropwise at 25° C. The mixture was stirred at 25° C. for 1 h. After completion of the reaction, the reaction mixture was diluted with water (5.00 L) and the organic layer was separated. The aqueous layer was re-extracted with CH₂Cl₂ (2.00 L). The combined organic layers were washed with a sat. aq. NaHCO₃ solution (1.50 L), brine (1.50 L), dried (Na₂SO₄), filtered and concentrated under vacuum. The crude residue was purified by normal phase chromatography (eluent: Petroleum ether/EtOAc from 100/1 to 10/1) to give the title compound as a yellow solid. ¹H NMR (600 MHz, DMSO-d₆) δ 8.04 (s, 1H), 7.81 (s, 1H), 5.88-5.79 (m, 1H), 3.92-3.83 (m, 1H), 3.80-3.68 (m, 1H), 2.53 (s, 3H), 2.40-2.32 (m, 1H), 2.06-1.99 (m, 1H), 1.99-1.93 (m, 1H), 1.77-1.69 (m, 1H), 1.60-1.56 (m, 2H). UPLC-MS-2b: Rt=1.32 min; MS m/z [M+H]⁺ 329.0/331.0/333.0.

### Step 7: Intermediate D1: 5-Chloro-6-methyl-1-(tetrahydro-2H-pyran-2-yl)-4-(4,4,5,5-tetramethyl-1,3,2-dioxaborolan-2-yl)-1H-indazole

A suspension of 4-bromo-5-chloro-6-methyl-1-(tetrahydro-2H-pyran-2-yl)-1H-indazole (Step 6, 450 g, 1.37 mol), KOAc (401 g, 4.10 mol) and B₂Pin₂ (520 g, 2.05 mol) in 1,4-dioxane (3.60 L) was degassed with nitrogen for 0.5 h. Pd(dppf)Cl₂·CH₂Cl₂ (55.7 g, 68.3 mmol) was added and the reaction mixture was stirred at 90° C. for 6 h. The reaction mixture was filtered through diatomite and the filtered cake was washed with EtOAc (1.50 L×3). The mixture was concentrated under vacuum to give a black oil which was purified by normal phase chromatography (eluent: Petroleum ether/EtOAc from 100/1 to 10/1) to give the desired product as brown oil. The residue was suspended in petroleum ether (250 mL) for 1 h to obtain a white precipitate. The suspension was filtered, dried under vacuum to give the title compound as a white solid. UPLC-MS-2a: Rt=1.27 min; MS m/z [M+H]⁺ 377.1/379.

### Intermediate D4: 5-Chloro-6-methyl-4-(4,4,5,5-tetramethyl-1,3,2-dioxaborolan-2-yl)-1-tosyl-1H-indazole

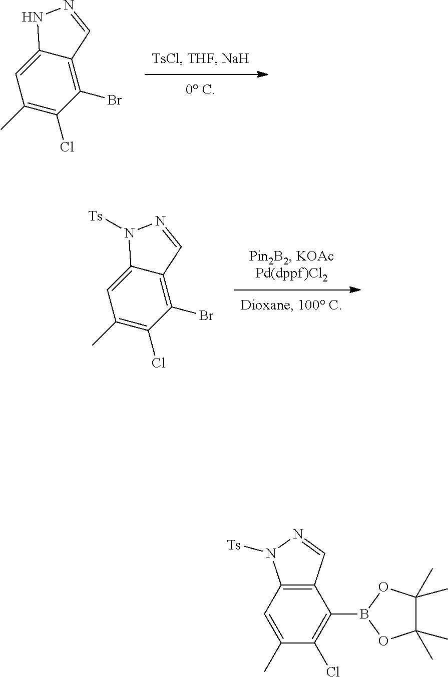 [SMILES: Cc1cc2[nH]ncc2c(Br)c1Cl ; Cc1ccc(S(=O)(=O)n2ncc3c(Br)c(Cl)c(C)cc32)cc1 ; Cc1ccc(S(=O)(=O)n2ncc3c(B4OC(C)(C)C(C)(C)O4)c(Cl)c(C)cc32)cc1]

### Step 1: 4-Bromo-5-chloro-6-methyl-1-tosyl-1H-indazole

A stirred solution of 4-bromo-5-chloro-6-methyl-1H-indazole (Intermediate D2, 240 g, 977 mmol) in THF (2.40 L) was treated with NaH (43.0 g, 1.08 mol, 60.0% in mineral oil) under nitrogen atmosphere and the reaction mixture was stirred at 0° C. for 30 min. Then the reaction mixture was treated with TsCl (195 g, 1.03 mol) and stirred at 0° C. for 1 h. The RM was quenched with water (1.00 L), diluted and extracted with EtOAc (1.00 L×3). The combined organic layers were washed with water, brine, dried (MgSO₄), filtered and concentrated under vacuum. The residue was suspended in MTBE (200 mL) for 20 min to give the title compound an off-white solid. ¹H NMR (400 MHz, DMSO-d₆) δ 8.49 (s, 1H), 8.16 (s, 1H), 7.91-7.83 (m, 2H), 7.47-7.37 (m, 2H), 2.61 (s, 3H), 2.35 (s, 3H). UPLC-MS-1a: Rt=1.42 min; MS m/z [M+H]⁺ 399.1/401.1/403.1.

### Step 2: 5-Chloro-6-methyl-4-(4,4,5,5-tetramethyl-1,3,2-dioxaborolan-2-yl)-1-tosyl-1H-indazole

A suspension of 4-bromo-5-chloro-6-methyl-1-tosyl-1H-indazole (Step 1, 370 g, 925 mmol), KOAc (272 g, 2.78 mol) and B₂Pin₂ (470 g, 1.85 mol) in 1,4-dioxane (3.00 L) was degassed with nitrogen for 30 min. Pd(dppf)Cl₂·CH₂Cl₂ (75.6 g, 92.6 mmol) was added and the reaction mixture was stirred at 100° C. for 4 h. The reaction mixture was filtered through diatomite and the filtered cake was washed with EtOAc (1.50 L). The filtrate was concentrated under vacuum to give a black oil which was filtered through silica gel and then the residue was suspended in EtOAc (500 mL) at 60° C. for 1 h. The mixture was cooled to 25° C. and a solid was precipitated out. The solid was filtered and dried under vacuum to give the title compound as a light yellow solid. ¹H NMR (600 MHz, DMSO-d₆) δ 8.45 (s, 1H), 8.21 (s, 1H), 7.81 (d, 2H), 7.39 (d, 2H), 2.53 (s, 3H), 2.33 (s, 3H), 1.34 (s, 12H); UPLC-MS-2c: Rt=1.27 min; MS m/z [M+H]⁺ 447.1/449.2.

### Intermediate D5: 5,6-Dimethyl-4-(4,4,5,5-tetramethyl-1,3,2-dioxaborolan-2-yl)-1-tosyl-1H-indazole

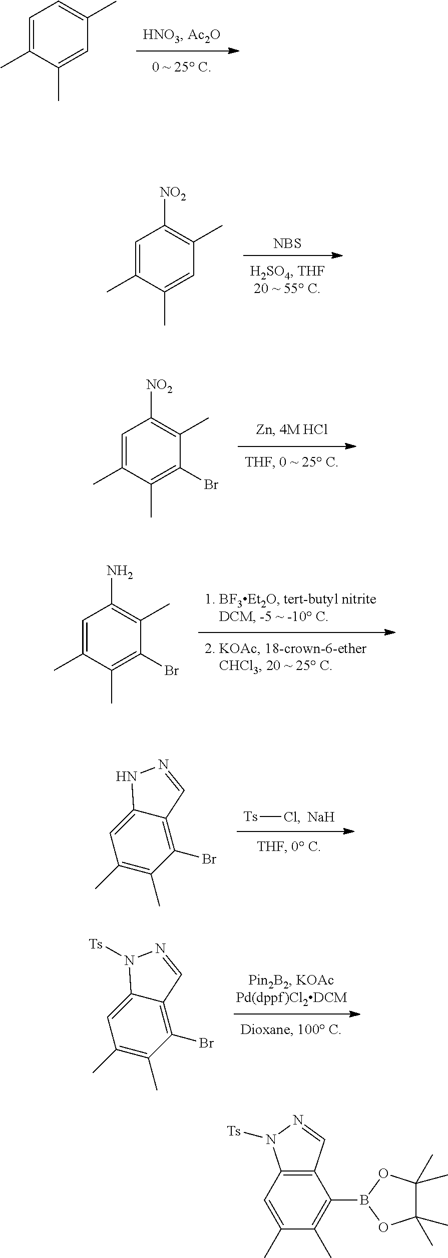

### Step 1: 1,2,4-Trimethyl-5-nitrobenzene

To an ice-cooled solution of 1,2,4-trimethylbenzene (1000 g, 8.32 mol) in Ac₂O (5.0 L) was added dropwise a cold solution of HNO₃ (847 g, 9.27 mol, 605 mL, 69.0% purity) in Ac₂O (1.00 L) by using a dropping funnel over a period of 30 min. The reaction mixture was then allowed to stir at 0° C. for 30 min. The RM was allowed to warm gradually to 25° C. and stirred for 2 h. The RM was poured slowly into H₂O (4.00 L) and stirred for 15 min. The aqueous phase was extracted with EtOAc (2.00 L×3). The combined organic phase was washed with brine (2.00 L×2), dried (Na₂SO₄), filtered and concentrated in vacuum. The crude product was purified by normal phase chromatography (eluent: Petroleum ether/EtOAc 100:1 to 100:5). ¹H NMR (400 MHz, CDCl₃) δ 7.80 (s, 1H), 7.08 (s, 1H), 2.55 (s, 3H), 2.30 (s, 3H), 2.29 (s, 3H).

### Step 2: 3-Bromo-1,2,4-trimethyl-5-nitrobenzene

To a cooled solution of 1,2,4-trimethyl-5-nitrobenzene (450 g, 2.72 mol) in TFA (1.58 L) was slowly added H₂SO₄ (450 mL) followed by portionwise addition of NBS (485 g, 2.72 mol) and the reaction mixture was heated at 55° C. for 2 h. The RM was quenched with crushed ice (4.00 kg) to obtain a brown solid which was filtered through vacuum, washed with cold water and dried under vacuum to give the title compound which was used into the next step without further purification. ¹H NMR (400 MHz, CDCl₃) δ 7.55 (s, 1H), 2.57 (s, 3H), 2.46 (s, 3H), 2.39 (s, 3H).

### Step 3: 3-Bromo-2,4,5-trimethylaniline

To an ice-cooled solution of 3-bromo-1,2,4-trimethyl-5-nitrobenzene (Step 2, 480 g, 1.97 mol) in THF (3.84 L) was added HCl (4M, 1.72 L) followed by Zinc (465 g, 7.10 mol) in small portions. The reaction mixture was allowed to warm slowly to 25° C. and stirred for 2 h. The RM was basified with an aq. sat NaHCO₃ solution until pH=8. The mixture was diluted with EtOAc (500 mL), stirred vigorously for 10 min and then filtered through a celite pad. The organic layer was separated and the aqueous layer was re-extracted with EtOAc (1.50 L×3). The combined organic layers were washed with brine, dried (Na₂SO₄), filtered and concentrated under reduced pressure to give the title compound which was used into the next step without further purification. ¹H NMR (400 MHz, CDCl₃) δ 6.49 (s, 1H), 3.71-3.40 (m, 2H), 2.31 (s, 3H), 2.29 (s, 3H), 2.24 (s, 3H).

### Step 4: 3-Bromo-2,4,5-trimethylbenzenediazonium tetrafluoroborate

The title compound was prepared by a method similar to that of Intermediate D1, Step 4 by replacing 3-bromo-4-chloro-2,5-dimethylaniline with 3-bromo-2,4,5-trimethylaniline (Step 3, 400 g, 1.87 mol). The title compound was used without further purification in the next step.

### Step 5: 4-Bromo-5,6-dimethyl-1H-indazole

The title compound was prepared by a method similar to that of Intermediate D1, Step 5 by replacing 3-bromo-4-chloro-2,5-dimethylbenzenediazonium tetrafluoroborate with 3-bromo-2,4,5-trimethylbenzenediazonium tetrafluoroborate (Step 4). The crude product was purified by normal phase chromatography (eluent: Petroleum ether/EtOAc=100/1 to 10/1). ¹H NMR (400 MHz, CDCl₃) δ 10.07 (br s, 1H), 8.00 (s, 1H), 7.24 (s, 1H), 2.46 (s, 6H).

### Step 6: 4-Bromo-5,6-dimethyl-1-tosyl-1H-indazole

To an ice-cooled solution of 4-bromo-5,6-dimethyl-1H-indazole (71.0 g, 315 mmol) in THF (710 mL) was added NaH (13.9 g, 347 mmol, 60% in mineral oil) under N₂ and the reaction mixture was stirred at 0° C. for 0.5 h followed by the addition of Ts-Cl (63.1 g, 331 mmol). The RM was stirred at 0° C. for 1 h, then poured into ice cold water (2.00 L), extracted with EtOAc (1.00 L×3). The combined organic layers were washed with brine (1.00 L), dried (Na₂SO₄), filtered and concentrated under reduced pressure. The crude residue was suspended in methyl t-butyl ether (500 mL) and stirred for 20 min to give the title after filtration the title compound as yellow solid. ¹H NMR (400 MHz, CDCl₃) δ 8.10 (s, 1H), 7.95 (m, 1H), 7.88-7.83 (m, 2H), 7.25 (m, 2H), 2.51 (s, 3H), 2.43 (s, 3H), 2.37 (s, 3H).

### Step 7: 5,6-Dimethyl-4-(4,4,5,5-tetramethyl-1,3,2-dioxaborolan-2-yl)-1-tosyl-1H-indazole

To a solution of 4-bromo-5,6-dimethyl-1-tosyl-1H-indazole (93.0 g, 245 mmol) in 1,4-dioxane (60.0 mL) was added KOAc (72.2 g, 736 mmol), Pin₂B₂ (74.7 g, 294 mmol) and Pd(dppf)Cl₂·CH₂Cl₂ (20.0 g, 24.5 mmol) under N₂. The reaction mixture was stirred at 100° C. for 12 h. The RM was filtered through diatomite and the filtered cake washed with EtOAc (1.50 L×3). The filtrate was concentrated under reduce pressure and the crude residue was purified by column chromatography (eluent: Petroleum ether/EtOAc=100/1 to 10/1). Then the solid was triturated with CH₂Cl₂ (100 mL) and MeOH (500 mL) at 35° C. for 30 min to give the title compound as a yellow solid. ¹H NMR (600 MHz, CDCl₃) δ. 8.51 (s, 1H), 8.09 (s, 1H), 7.89-7.76 (m, 2H), 7.27-7.17 (m, 2H), 2.55 (s, 3H), 2.47 (s, 3H), 2.36 (s, 3H), 1.40 (s, 12H). UPLC-MS-2b: Rt=1.30 min; MS m/z [M+H]⁺ 427.3.

### Intermediate D6: 5,6-Dichloro-1-(tetrahydro-2H-pyran-2-yl)-4-(4,4,5,5-tetramethyl-1,3,2-dioxaborolan-2-yl)-1H-indazole

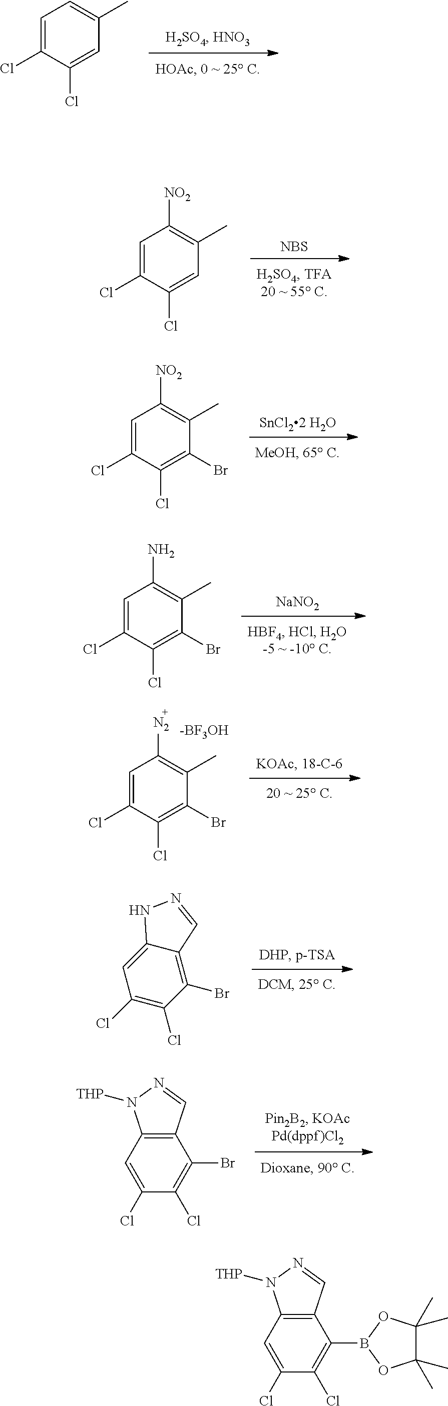

### Step 1: 1,2-Dichloro-4-methyl-5-nitrobenzene

The title compound was prepared by a method similar to Step 1 in the synthesis of Intermediate D1 by replacing 2-chloro-1,4-dimethylbenzene with 1,2-dichloro-4-methylbenzene. ¹H NMR (400 MHz, CDCl₃) δ 8.08 (s, 1H), 7.43 (s, 1H), 2.56 (s, 3H).

### Step 2: 3-Bromo-1,2-dichloro-4-methyl-5-nitrobenzene

The title compound was prepared by a method similar to Step 2 in the synthesis of Intermediate D1 by replacing 1-chloro-2,5-dimethyl-4-nitrobenzene with 1,2-dichloro-4-methyl-5-nitrobenzene. ¹H NMR (400 MHz, CDCl₃) δ 7.90 (s, 1H), 2.62 (s, 3H).

### Step 3: 3-Bromo-4,5-dichloro-2-methylaniline

To a solution of 3-bromo-1,2-dichloro-4-methyl-5-nitrobenzene (Step 2, 3.45 kg, 12.1 mol) in MeOH (27.0 L) was added SnCl₂·2H₂O (8.20 kg, 36.3 mol) and the reaction mixture was stirred at 65° C. for 10 h. TLC (petroleum ether:EtOAc=5:1) indicated consumption of the starting material (R_{f}=0.49). The pH of the mixture was adjusted to pH=8 by addition of 20.0% aq. NaOH solution (10.0 L) at 0-10° C. and the mixture was extracted with EtOAc (5.00 L×8). The combined organic layers were washed with brine (5.00 L×2), dried (Na₂SO₄), filtered and concentrated under vacuum. The crude residue was triturated with petroleum ether (2.00 L) at 25° C. for 12 h to give the title compound as a yellow solid. ¹H NMR (400 MHz, CDCl₃) δ 6.77 (s, 1H), 3.61-3.89 (m, 2H), 2.30 (s, 3H).

### Step 4: 3-Bromo-4,5-dichloro-2-methylbenzenediazonium trifluoro(hydroxy)borate

To an ice-cooled solution of 3-bromo-4,5-dichloro-2-methylaniline (Step 3, 1.70 kg, 6.67 mol) in HCl (6M, 17.4 L, 105 mmol) stirred for 30 min was added dropwise an ice-cooled solution of NaNO₂ (506 g, 7.34 mol) in H₂O (1.20 L) while maintaining a temperature at 0° C. The resulting mixture was stirred for 1 h. HBF₄ (9.22 kg, 42.0 mol, 6.54 L, 40.0% purity) was added dropwise and the reaction mixture was stirred at 0° C. for 30 min. TLC (petroleum ether:EtOAc=5:1) indicated consumption of the starting material (R_{f}=0.39). The resulting precipitate was filtered through vacuum, washed with cold water (2.00 L) and MTBE (2.00 L) and then dried under vacuum to obtain the diazonium salt as a pale yellow solid. The crude product was triturated with MTBE (1.00 L) at 25° C. for 30 min to give the title compound as a yellow solid which was used without further purification in the next step.

### Step 5: 4-Bromo-5,6-dichloro-1H-indazole

The title compound was prepared by a method similar to that of Intermediate D1, Step 5 by replacing 3-bromo-4-chloro-2,5-dimethylbenzenediazonium tetrafluoroborate with 3-bromo-4,5-dichloro-2-methylbenzenediazonium trifluoro(hydroxy)borate. ¹H NMR (400 MHz, DMSO-d₆) δ 13.71 (br s, 1H), 8.10 (s, 1H), 7.94 (s, 1H); UPLC-MS-1c: Rt=1.18 min; MS m/z [M+H]⁻ 262.9/264.9/266.8/268.8.

### Step 6: 4-Bromo-5,6-dichloro-1-(tetrahydro-2H-pyran-2-yl)-1H-indazole

The title compound was prepared by a method similar to Step 6 in the synthesis of Intermediate D1 by replacing 4-bromo-5-chloro-6-methyl-1H-indazole with 4-bromo-5,6-dichloro-1H-indazole. ¹H NMR (400 MHz, DMSO-d₆) δ 8.22 (s, 1H), 8.13 (s, 1H), 5.91-5.88 (m, 1H), 3.90-3.83 (m, 1H), 3.81-3.72 (m, 1H), 2.40-2.27 (m, 1H), 2.07-1.92 (m, 2H), 1.78-1.65 (m, 1H), 1.62-1.52 (m, 2H); UPLC-MS-1c: Rt=1.55 min; MS m/z [M+H]⁺ 349.1/351.0/353.0.

### Step 7: 5,6-Dichloro-1-(tetrahydro-2H-pyran-2-yl)-4-(4,4,5,5-tetramethyl-1,3,2-dioxaborolan-2-yl)-1H-indazole

The title compound was prepared by a method similar to Step 7 in the synthesis of Intermediate D1, Step 7 by replacing 4-bromo-5-chloro-6-methyl-1-(tetrahydro-2H-pyran-2-yl)-1H-indazole with 4-bromo-5,6-dichloro-1-(tetrahydro-2H-pyran-2-yl)-1H-indazole. ¹H NMR (400 MHz, DMSO-d₆) δ 8.23 (s, 1H), 8.16 (s, 1H), 5.90-5.87 (m, 1H), 3.89-3.71 (m, 2H), 2.41-2.28 (m, 1H), 2.07-1.90 (m, 2H), 1.78-1.65 (m, 1H), 1.62-1.52 (m, 2H), 1.38 (s, 12H); UPLC-MS-1c: Rt=1.53 min; MS m/z [M+H]⁺ 395.3/397.3/399.3.

### Intermediate D7: 5-Chloro-1-(tetrahydro-2H-pyran-2-yl)-4-(4,4,5,5-tetramethyl-1,3,2-dioxaborolan-2-yl)-1H-indazole

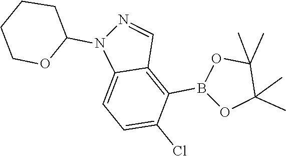 [SMILES: CC1(C)OB(c2c(Cl)ccc3c2cnn3C2CCCCO2)OC1(C)C]

### Step 1: 4-Bromo-5-chloro-1-(tetrahydro-2H-pyran-2-yl)-1H-indazole

To a solution of 4-bromo-5-chloro-1H-indazole (250 g, 1.08 mol) in CH₂Cl₂ (2.50 L) were added pTSA (9.30 g, 54.0 mmol) followed by DHP (273 g, 3.24 mol, 296 mL). The reaction mixture was stirred at 25° C. for 1 h. The RM was poured into a sat. aq. NaHCO₃ solution (1 L), washed with brine and dried (Na₂SO₄), filtered and concentrated in vacuum. The crude product was triturated in Petroleum ether (500 mL) and collected by filtration to give the title product. UPLC-MS-1a: Rt=1.28 min; MS m/z [M+H]⁺ 315/317/319.

### Step 2: 5-Chloro-1-(tetrahydro-2H-pyran-2-yl)-4-(4,4,5,5-tetramethyl-1,3,2-dioxaborolan-2-yl)-1H-indazole

A solution of 4-bromo-5-chloro-1-(tetrahydro-2H-pyran-2-yl)-1H-indazole (step 1, 320 g, 1.01 mol), B₂Pin₂ (283 g, 1.12 mol) and KOAc (299 g, 3.04 mol) in 1,4-dioxane (1.39 L) was degassed with N². Then Pd(dppf)Cl₂·CH₂Cl₂ (24.8 g, 30.4 mmol) was added and the reaction mixture was stirred at 110° C. for 18 h. The mixture was filtered, the cake was washed with EtOAc (10 L) and the filtrate was concentrated in vacuum. The crude product was purified by normal phase chromatography (eluent: EtOAc in petroleum ether from 0 to 50%) to give the title compound. UPLC-MS-1a: Rt=1.35 min; MS m/z [M+H]⁺ 363/365.

### Intermediate D8: 6-Chloro-5-methoxy-1-(tetrahydro-2H-pyran-2-yl)-4-(4,4,5,5-tetramethyl-1,3,2-dioxaborolan-2-yl)-1H-indazole

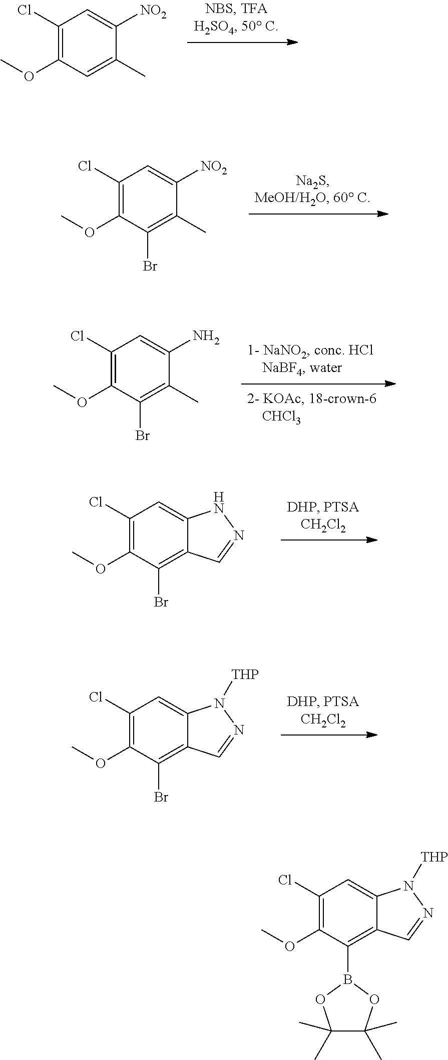

### Step 1: 3-Bromo-1-chloro-2-methoxy-4-methyl-5-nitrobenzene

To a suspension of 2-chloro-5-methyl-4-nitroanisole (10.0 g, 49.6 mmol) in TFA (30 mL) and conc. H₂SO₄ (10 mL) at 0° C., was added N-bromosuccinimide (8.76 g, 49.6 mmol) portion wise, under nitrogen atmosphere. The reaction mixture was stirred at 50° C. for 2 h. After completion of the reaction, the RM was quenched with a cold bicarbonate water solution to adjust pH 6-7 and extracted with Et₂O. The combined organic layer was washed by brine, dried (Na₂SO₄), filtered concentrated under reduced pressure. The crude residue was directly used in the next step without any further purification. ¹H NMR (400 MHz, CDCl₃) δ 7.91 (s, 1H), 3.96 (s, 3H), 2.61 (s, 3H).

### Step 2: 3-Bromo-5-chloro-4-methoxy-2-methylaniline

3-Bromo-1-chloro-2-methoxy-4-methyl-5-nitrobenzene (Step 1, 12.2 g, 43.57 mmol) was dissolved in (MeOH:H₂O) (70 mL: 30 mL), sodium sulphide (10.2 g, 130.7 mmol) was added portion wise at RT and the reaction mixture was stirred for 16 h at 60° C. After completion of the reaction, the RM was quenched with cold water, filtered through a celite pad and the filtrate was extracted by EtOAc. The combined organic layer was washed with brine, dried (Na₂SO₄), filtered and concentrated under reduced pressure. The crude residue was purified by reverse phase chromatography (eluent: 65 to 70% CH₃CN in H₂O containing 0.1% formic acid) to give the title product as yellow solid. ¹H NMR (400 MHz, DMSO) δ 6.72 (s, 1H), 5.24 (s, 2H), 3.65 (s, 3H), 2.14 (s, 3H).

### Step 3: 4-Bromo-6-chloro-5-methoxy-1H-indazole

3-Bromo-5-chloro-4-methoxy-2-methylaniline (Step 2, 6.80 g, 27.2 mmol) was dissolved in THF (60 mL) and cooled to 0° C., HCl (6N, 68 mL) was added drop wise and the mixture was stirred for 10 min. Then NaNO₂ (2.27 g, 32.6 mmol) in a minimum amount of water was added drop wise and the reaction mixture was stirred at 0° C. for 15 min. HBF₄ (50% in water, 21 mL) was added drop wise and the RM was stirred at 0° C. for 30 min. A precipitate formed and was filtered, washed with cold water, with hexane and then was dried under vacuum to give the diazonium salt intermediate as a yellow solid. The solid was dissolved in chloroform (100 mL), the solution was cooled to 0° C. and 18-crown-6 ether (7.26 g, 27.2 mmol) followed by potassium acetate (4.04 g, 41.3 mmol) were added portion wise. The reaction mixture was stirred at 0° C. for 30 min. After completion of the reaction, the RM was poured into CH₂Cl₂ and washed with water and brine. The organic layer was dried (Na₂SO₄), filtered and concentrated under vacuum. The crude residue was purified by normal phase chromatography (eluent: 20% EtOAc in hexane) to give the title product as light yellow solid. ¹H NMR (400 MHz, DMSO) δ 13.5 (s, 1H), 8.03 (s, 1H), 7.77 (s, 1H), 3.82 (s, 3H).

### Step 4: 4-Bromo-6-chloro-5-methoxy-1-(tetrahydro-2H-pyran-2-yl)-1H-indazole

4-Bromo-6-chloro-5-methoxy-1H-indazole (5.10 g, 19.5 mmol) was dissolved in CH₂Cl₂ (100 mL) and cooled to 0° C. pTSA (0.18 g, 0.98 mmol) was added, followed by DHP (4.92 g, 58.6 mmol) at 0° C. and the reaction mixture was stirred at RT for 2 h. After completion of the reaction, the RM was diluted with CH₂Cl₂, washed with water, brine and dried (Na₂SO₄), filtered and concentrated under vacuum. The crude residue was purified by normal phase chromatography (eluent: 5 to 7% EtOAc in hexane) to give the title product as orange oil. ¹H NMR (400 MHz, CDCl₃) δ 8.01 (s, 1H), 7.69 (s, 1H), 5.99-5.69 (m, 1H), 3.96 (s, 3H) 3.45-3.43 (m, 2H), 2.53-2.51 (m, 1H), 2.17-2.10 (m, 1H), 1.94-1.93 (m, 2H), 1.79-1.73 (m, 2H).

### Step 5: 6-Chloro-5-methoxy-1-(tetrahydro-2H-pyran-2-yl)-4-(4,4,5,5-tetramethyl-1,3,2-dioxaborolan-2-yl)-1H-indazole

4-Bromo-6-chloro-5-methoxy-1-(tetrahydro-2H-pyran-2-yl)-1H-indazole (3.60 g, 10.4 mmol), B₂Pin₂ (13.2 g, 52.2 mmol), KOAc (3.07 g, 31.3 mmol) and PdCl₂dppf·CH₂Cl₂ (0.11 g, 0.16 mmol) were dissolved in dry 1,4-dioxane pre-degassed with nitrogen gas at RT. The reaction mixture was stirred at 120° C. for 7 h. After completion of the reaction, the RM was diluted with EtOAc, filtered through a celite pad and the filtrate was concentrated under reduce pressure. The crude residue was purified by reverse phase chromatography (eluent: 65 to 76% CH₃CN in H₂O containing 0.025% NH₃) to give the title product as a mixture of boronate ester and boronic acid (1:1) as brown solid. ¹H NMR (400 MHz, CDCl₃) δ 8.51 (s, 1H), 8.15 (s, 0.5H), 8.09 (s, 0.5H), 7.97 (s, 0.5H), 7.92 (s, 0.5H), 5.86-5.81 (m, 1H), 3.87-3.72 (m, 2H), 3.72 (s, 1.5H), 3.77 (s, 1.5H), 2.03-2.00 (m, 1H), 1.94-1.91 (m, 2H), 1.72 (m, 1H), 1.57 (m, 2H), 1.41-1.37 (s, 6H).

### Intermediate D9: 5-Chloro-6-fluoro-1-(tetrahydro-2H-pyran-2-yl)-4-(4,4,5,5-tetramethyl-1,3,2-dioxaborolan-2-yl)-1H-indazole

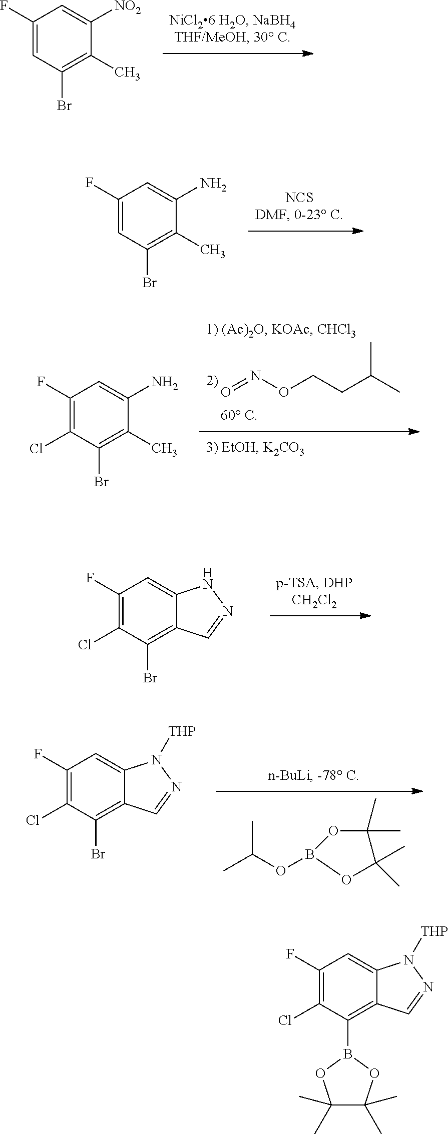

### Step 1: 3-Bromo-5-fluoro-2-methylaniline

To a solution of NiCl₂·6H₂O (12.2 kg, 51.0 mol) in THF (10.4 L) and MeOH (31.5 L) was added 1-bromo-5-fluoro-2-methyl-3-nitrobenzene (3.98 kg, 17.0 mol) and the mixture was cooled to 25-30° C. NaBH₄ (2.00 kg, 53.0 mol) was then added portion wise over 3 h and the reaction mixture was stirred at 30° C. for 30 min then at RT for another 30 min. After completion of the reaction, the RM was poured into ice-water filtered through celite and the filtrate was extracted with EtOAc. The combined organic layers were washed with brine, dried (Na₂SO₄), filtered and concentrated under vacuum to give the title compound which was used without purification in the next step. ¹H NMR (400 MHz, DMSO-d₆) δ 6.80-6.60 (m, 1H), 6.42-6.46 (m, 1H), 5.56 (br s, 2H), 2.10 (s, 3H).

### Step 2: 3-Bromo-4-chloro-5-fluoro-2-methylaniline

To an ice-cooled solution of 3-bromo-5-fluoro-2-methylaniline (Step 1, 3.17 kg, 15.5 mol) in DMF (32 L) was added NCS (2.67 kg, 15.5 mol) portionwise and the reaction mixture was stirred at RT for 48 h. After completion of the reaction, the RM was poured into ice cold water (100 L) and extracted with MTBE (3×20 L). The combined organic layers were washed with water then brine, dried (Na₂SO₄), filtered and concentrated under vacuum. The crude product was purified by normal phase chromatography (eluent: EtOAc in n-hexane 0 to 3%) to give the title compound. ¹H NMR (400 MHz, DMSO-d₆) δ 6.62 (d, 1H), 5.78 (br s, 2H), 2.18 (s, 3H).

### Step 3: 4-Bromo-5-chloro-6-fluoro-1H-indazole

A mixture of 3-bromo-4-chloro-5-fluoro-2-methylaniline (Step 2, 2.01 kg, 8.43 mol) and KOAc (836 g, 8.52 mol) in CHCl₃ (20 L) was treated with Ac₂O (2.51 kg, 24.6 mol) at 25-30° C. The reaction mixture was stirred at RT for 50 min. Isoamyl nitrite (1.48 kg, 12.6 mol) was added in portions over 12 h. After completion of the reaction, the RM was then refluxed at 62° C. The RM was poured into water (3.00 L) and extracted with CHCl₃ (3×1.00 L). The combined organic layers were washed with sodium carbonate (aq.) then brine, dried (Na₂SO₄), filtered and concentrated under vacuum. To the residue in EtOH (25.0 L) was added K₂CO₃ (3.01 g, 21.8 mol). The reaction mixture was stirred at RT for 12 h. The solid was filtered out and the filtrate was concentrated under vacuum. The mixture was poured into water (3.00 L) and the resulting solution was extracted with EtOAc (3×5.00 L). The combined organic layers were washed with sodium carbonate (aq.) and brine, dried and concentrated under vacuum to give a crude. The crude product was purified by normal phase chromatography (eluent: EtOAc/hexane 1/30) to give the title compound. ¹H NMR (400 MHz, DMSO-d₆) δ 13.7 (s, 1H), 8.10 (s, 1H), 7.70-7.70 (m, 1H).

### Step 4: 4-Bromo-5-chloro-6-fluoro-1-(tetrahydro-2H-pyran-2-yl)-1H-indazole

The title compound was prepared by a method similar to that of Intermediate D1 Step 6 by replacing 4-bromo-5-chloro-6-methyl-1H-indazole with 4-bromo-5-chloro-6-fluoro-1H-indazole. ¹H NMR (400 MHz, DMSO-d₆) δ 8.23-8.07 (m, 1H), 8.06-7.84 (m, 1H), 5.95-5.72 (m, 1H), 3.96-3.83 (m, 1H), 3.81-3.67 (m, 1H), 2.38-2.26 (m, 1H), 2.09-1.92 (m, 2H), 1.81-1.66 (m, 1H), 1.64-1.51 (m, 2H).

### Step 5: 5-Chloro-6-fluoro-1-(tetrahydro-2H-pyran-2-yl)-4-(4,4,5,5-tetramethyl-1,3,2-dioxaborolan-2-yl)-1H-indazole

To a well stirred solution of 4-bromo-5-chloro-6-fluoro-1-(tetrahydro-2H-pyran-2-yl)-1H-indazole (Step 4, 800 g, 2.40 mol) in THF (12.0 L) was added dropwise at −70° C./−78° C. n-BuLi (1.71 L, 4.28 mol). The reaction mixture was stirred at −78° C. for 1 h then 2-isopropoxy-4,4,5,5-tetramethyl-1,3,2-dioxaborolane (1.97 kg, 10.6 mol) was added dropwise at −78° C. with stirring. After the addition, the reaction mixture was stirred at −70° C./−60° C. for 1 h. The RM was quenched with 10% aq. citric acid solution (12.0 L) and extracted with EtOAc (2×5.00 L). The combined organic layers were washed with water then brine, dried and concentrated under vacuum. The crude product was recrystallized at −10° C./−20° C. with heptane. The solid was filtered and washed with heptane to give the title compound. ¹H NMR (400 MHz, CDCl₃) δ 8.24 (s, 1H), 7.48 (d, 1H), 5.70-5.65 (m, 1H), 4.02-3.95 (m, 1H), 3.76-3.72 (m, 1H), 2.52-2.47 (m, 1H), 2.17-2.08 (m, 2H), 1.77-1.69 (m, 3H), 1.43 (s, 12H).

### Intermediate D10: 6-Chloro-5-methyl-1-(tetrahydro-2H-pyran-2-yl)-4-(4,4,5,5-tetramethyl-1,3,2-dioxaborolan-2-yl)-1H-indazole

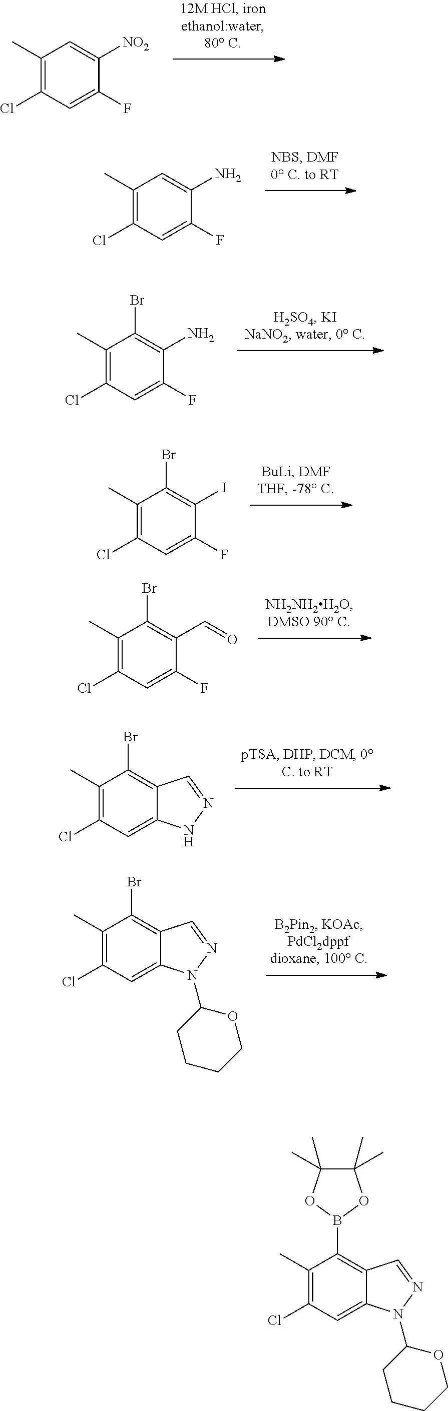

### Step 1: 4-Chloro-2-fluoro-5-methylaniline

To a solution of 1-chloro-5-fluoro-2-methyl-4-nitrobenzene (100 g, 527 mmol) in Ethanol:Water (1:1) (950 mL) was added dropwise HCl (12M, 41.8 mL, 527 mmol) at RT. The mixture was heated to 80° C. and Fe powder (83.9 g, 1.58 mol) was added slowly over a period of 30 min. The reaction mixture was stirred at 80° C. for 1 h. Then was cooled to RT, diluted with EtOAc and basified to pH=8-9 by addition of a sat. aq. NaHCO₃ solution. The layers were filtered over a pad of celite and the aqueous layer was extracted with EtOAc. The combined organic extracts were washed with brine, dried (Na₂SO₄) and concentrated under vacuum to give the title compound which was directly used in the next step without further purification. ¹H NMR (400 MHz, DMSO-d₆) δ 7.10 (d, 1H), 6.70 (d, 1H), 5.20 (s, 2H), 2.16 (s, 3H).

### Step 2: 2-Bromo-4-chloro-6-fluoro-3-methylaniline

4-Chloro-2-fluoro-5-methylaniline (Step 1, 78.0 g, 489 mmol) was dissolved in DMF (830 mL) and cooled to 0° C. NBS (36.2 g, 532 mmol) was added portion wise and the reaction mixture was stirred at RT for 1 h. The reaction mixture was poured into ice/water and extracted with EtOAc. The combined organic extracts were washed with brine, dried (Na₂SO₄), filtered concentrated under vacuum and the crude residue was purified by normal phase chromatography (eluent: EtOAc in hexane 0-2%) to give the title compound. ¹H NMR (400 MHz, DMSO-d₆) δ 7.32 (d, 1H), 5.46 (s, 2H), 2.37 (s, 3H).

### Step 3: 3-Bromo-1-chloro-5-fluoro-4-iodo-2-methylbenzene

To a solution of concentrated H₂SO₄ (200 mL) in water (700 mL) was added 2-bromo-4-chloro-6-fluoro-3-methylaniline (Step 2, 86.0 g, 356 mmol). The mixture was stirred at 25° C. for 10 min then cooled to 0° C. and NaNO₂ (26.4 g, 389 mmol) in water (100 mL) was added dropwise. The reaction mixture was stirred at 0-5° C. for 30 min. A solution of KI (231 g, 1.39 mol) in water (300 mL) was added dropwise to the reaction mixture which was stirred at 0° C. for another 20 min then allowed to reach RT and further stirred for 18 h. The RM was pourred into ice/water and extracted with EtOAc. The combined organic extracts were washed with a sat. aq. Na₂S₂O₃ solution, with a sat. aq. NaHCO₃ solution, brine, dried (Na₂SO₄), filtered and concentrated under vacuum. The crude residue was purified by normal phase chromatography (eluent: 100% hexane) to give the title compound as yellow solid. ¹H NMR (400 MHz, CDCl₃) δ 7.21 (d, 1H), 2.69 (s, 3H).

### Step 4: 2-Bromo-4-chloro-6-fluoro-3-methylbenzaldehyde

To a solution of 3-bromo-1-chloro-5-fluoro-4-iodo-2-methylbenzene (Step 3, 71.0 g, 206 mmol) in THF (710 mL) was added dropwise n-BuLi (23% in hexane, 84.8 mL, 305 mmol) at −78° C. over a period of 30 min and the mixture was stirred at −78° C. for another 30 min. Anhydrous DMF (22.3 g, 305 mmol) was added dropwise at −78° C. and the reaction mixture was stirred for 30 min. After completion of the reaction, the RM was quenched with HCl (1N, 55 mL). Water was added and the mixture was extracted with EtOAc. The combined organic extracts were washed with brine, dried (Na₂SO₄), filtered and concentrated under vacuum to give the title compound as an orange oil which was directly used in the next step without further purification. ¹H NMR (400 MHz, MeOD) δ 10.29 (s, 1H), 7.48 (d, 1H), 2.50 (s, 3H).

### Step 5: 4-Bromo-6-chloro-5-methyl-1H-indazole

2-Bromo-4-chloro-6-fluoro-3-methylbenzaldehyde (50.0 g, 199 mmol) was dissolved in DMSO (500 mL) and cooled to 0° C. NH₂NH₂·H₂O (119 g, 2.38 mmol) was added dropwise over a period of 30 min and the reaction mixture was stirred at 90° C. for 12 h. After completion of the reaction, the RM was diluted with water and extracted with EtOAc. The combined organic extracts were washed with brine, dried (Na₂SO₄), filtered, concentrated under reduced pressure and the crude residue was purified by trituration in n-pentane to give the title compound. ¹H NMR (400 MHz, CDCl₃) δ 8.07 (s, 1H), 7.56 (s, 1H), 2.63 (s, 3H). UPLC-MS-5: Rt=1.88 min; MS m/z [M+H]⁺ 245.2/247.2.

### Step 6: 4-Bromo-6-chloro-5-methyl-1-(tetrahydro-2H-pyran-2-yl)-1H-indazole

To a solution of 4-bromo-6-chloro-5-methyl-1H-indazole (Step 5, 31.0 g, 126 mmol) in CH₂Cl₂ cooled to 0° C. was slowly added pTSA (2.40 g, 12.6 mmol) followed by dropwise addition of DHP (31.8 g, 378 mmol) and the reaction mixture was stirred at RT for 3 h. After completion of the reaction, the RM was diluted with water and extracted with EtOAc. The combined organic extracts were washed with brine, dried (Na₂SO₄), filtered, concentrated under vacuum and the crude residue was purified by normal phase chromatography (eluent: 3-5% EtOAc in hexane) to give the title compound. ¹H NMR (400 MHz, CDCl₃) δ 7.98 (s, 1H), 7.67 (s, 1H), 5.68 (m, 1H), 4.05 (m, 1H), 3.89 (m, 1H), 2.62 (s, 3H), 2.53 (m, 1H), 2.17-2.09 (m, 2H), 1.93 (m, 1H), 1.70 (m, 2H). UPLC-MS-5: Rt=2.33 min, MS m/z [M-THP]⁺244/246.

### Step 6: 6-Chloro-5-methyl-1-(tetrahydro-2H-pyran-2-yl)-4-(4,4,5,5-tetramethyl-1,3,2-dioxaborolan-2-yl)-1H-indazole

6-Chloro-5-methyl-1-(tetrahydro-2H-pyran-2-yl)-4-(4,4,5,5-tetramethyl-1,3,2-dioxaborolan-2-yl)-1H-indazole (Step 5, 38.0 g, 115 mmol), B₂Pin₂ (32.2 g, 126 mmol) and KOAc (35.9 g, 367 mmol) were added in 1,4-dioxane (320 mL) and the reaction mixture was degassed with N₂ for 10 min. PdCl₂dppf (4.21 g, 5.76 mmol) was added and the RM was stirred at 100° C. for 20 h in a sealed flask. The RM was cooled to RT, diluted with EtOAc and filtered through celite. The filtrate was concentrated under vacuum and the crude residue was purified by normal phase chromatography (eluent: 3-5% EtOAc in hexane) followed by trituration in n-pentane to give the title compound as a white solid. ¹H NMR (400 MHz, DMSO-d₆) δ 8.18 (s, 1H), 8.01 (s, 1H), 5.84 (m, 1H), 3.90-3.82 (m, 1H), 3.79-3.71 (m, 1H), 2.59 (s, 3H), 2.41-2.31 (m, 1H), 2.06-1.98 (m, 1H), 1.96-1.89 (m, 1H), 1.77-1.67 (m, 1H), 1.61-1.53 (m, 2H), 1.37 (s, 12H). UPLC-MS-5: Rt=2.54 min, MS m/z [M+H]⁺ 377.4/379.4.

### Intermediate D11: Tert-butyl 3-amino-5-chloro-6-methyl-4-(4,4,5,5-tetramethyl-1,3,2-dioxaborolan-2-yl)-1H-indazole-1-carboxylate

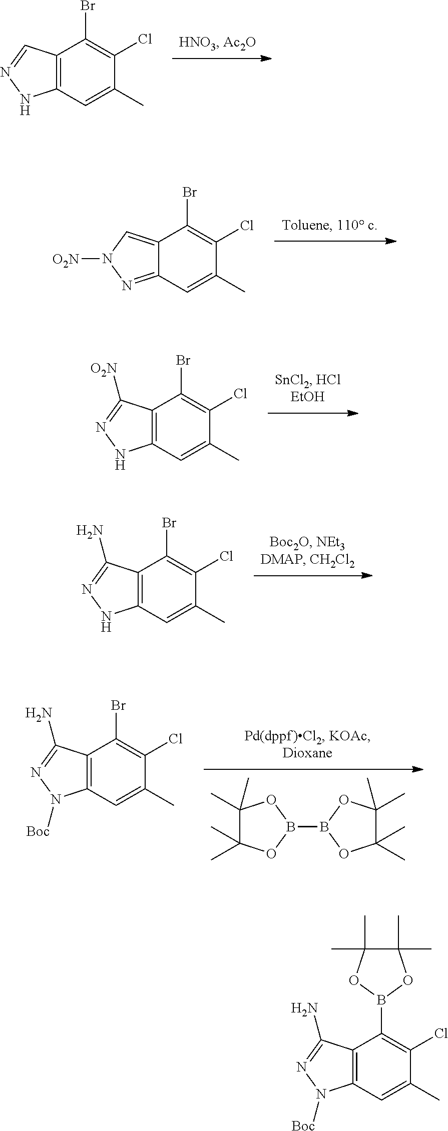

### Step 1: 4-Bromo-5-chloro-6-methyl-2-nitro-2H-indazole

To a solution of fuming nitric acid (3.64 mL, 81 mmol) in acetic anhydride (100 mL) was added at 0° C. 4-bromo-5-chloro-6-methyl-1H-indazole (Intermediate D2, 5.00 g, 20.4 mmol) and the reaction mixture was stirred at 0° C. for 30 min. The RM was poured into a mix of ice/water and the precipitate was filtered off, washed with water and dried under reduced pressure to give the title compound. ¹H NMR (400 MHz, DMSO-d₆) δ 9.35 (s, 1H), 7.78 (s, 1H); UPLC-MS-3: Rt=1.36 min; MS m/z [M−H]⁻ 288.0/290.0.

### Step 2: 4-Bromo-5-chloro-6-methyl-3-nitro-1H-indazole

A solution of 4-bromo-5-chloro-6-methyl-2-nitro-2H-indazole (Step 1, 5.55 g, 17.4 mmol) in toluene (100 mL) was warmed to 110° C. and stirred for 1 h. The reaction mixture was cooled to RT and the solid was filtered off, washed with toluene and dried under reduced pressure to give the title compound. ¹H NMR (400 MHz, DMSO-d₆) δ 14.6 (br s, 1H), 7.79 (s, 1H), 2.57 (s, 3H); UPLC-MS-3: Rt=1.13 min; MS m/z [M−H]⁻ 288.0/290.0.

### Step 3: 4-Bromo-5-chloro-6-methyl-1H-indazol-3-amine

To a suspension of 4-bromo-5-chloro-6-methyl-3-nitro-1H-indazole (Step 2, 4.19 g, 14.3 mmol) in EtOH (160 mL) and hydrochloric acid (10.5N, 27.1 mL, 286 mmol) was added tin(II) chloride (13.5 g, 71.4 mmol) and the reaction mixture was stirred at RT for 2 h. The RM was concentrated under reduced pressure, the white residue was diluted with CH₂Cl₂ (150 mL) and water (200 mL), cooled to 0° C. and basified with solid NaOH until pH 9. The cloudy mixture was extracted with CH₂Cl₂ (3×500 mL), the organic phase dried (phase separator) and concentrated under reduced pressure to give the title compound as a light pink cotton. ¹H NMR (400 MHz, DMSO-d₆) δ 11.9 (s, 1H), 7.26 (s, 1H), 5.19 (s, 2H), 2.44 (s, 3H); UPLC-MS-3: Rt=0.88 min; MS m/z [M+H]⁺ 260.1/262.0.

### Step 4: Tert-butyl 3-amino-4-bromo-5-chloro-6-methyl-1H-indazole-1-carboxylate

To a suspension of 4-bromo-5-chloro-6-methyl-1H-indazol-3-amine (Step 3, 3.25 g, 12.5 mmol), triethylamine (3.48 mL, 24.95 mmol) and DMAP (0.38 g, 3.12 mmol) in CH₂Cl₂ (70 mL) was added Boc-anhydride (3.13 g, 14.4 mmol) and the reaction mixture was stirred at RT for 2 h. A precipitate formed, the RM was filtered to give tert-butyl 3-amino-4-bromo-5-chloro-6-methyl-1H-indazole-1-carboxylate as the major product: ¹H NMR (400 MHz, DMSO-d6) δ 8.03 (s, 1H), 6.10 (s, 2H), 2.52 (s, 3H), 1.59 (s, 9H); UPLC-MS-3: Rt=1.30 min; MS m/z [M+H]⁺ 360.0/362.0. The filtrate was concentrated under reduced pressure, the residue dissolved in EtOAc, washed with a sat. aq. NaHCO₃ solution and brine. The organic phase was dried (phase separator), concentrated under reduced pressure and the crude residue was purified by normal phase chromatography (eluent: c-hexane/EtOAc 100/0 to 70/30) to give tert-butyl 3-amino-4-bromo-5-chloro-6-methyl-2H-indazole-2-carboxylate: ¹H NMR (400 MHz, DMSO-d₆) δ 7.21 (s, 1H), 7.03 (s, 2H), 2.36 (s, 3H), 1.60 (s, 9H); UPLC-MS-3: Rt=1.31 min; MS m/z [M+H]⁺ 360.1/362.1.

### Step 5: Tert-butyl 3-amino-5-chloro-6-methyl-4-(4,4,5,5-tetramethyl-1,3,2-dioxaborolan-2-yl)-1H-indazole-1-carboxylate

In a sealed tube, a solution of tert-butyl 3-amino-4-bromo-5-chloro-6-methyl-1H-indazole-1-carboxylate (Step 4, 1.00 g, 2.77 mmol), bis(pinacolato)diboron (2.82 g, 11.1 mmol), PdCl₂(dppf) (0.20 g, 0.28 mmol) and potassium acetate (0.68 g, 6.93 mmol) in 1,4-dioxane (24 mL) was stirred at 80° C. for 16 h. Bis(pinacolato)diboron (2.82 g, 11.1 mmol) and PdCl₂(dppf) (0.20 g, 0.28 mmol) were added and the reaction mixture was stirred for an additional 13.5 h. The RM was filtered, the filtrate concentrated under reduced pressure and the crude residue purified by normal phase chromatography (eluent: c-hexane/EtOAc 100/0 to 40/60) to give the title compound. 1H NMR (400 MHz, DMSO-d₆) δ 7.92 (s, 2H), 6.35 (s, 1H), 2.45 (s, 3H), 1.59 (s, 9H), 1.40 (s, 12H); UPLC-MS-3: Rt=1.43 min; MS m/z [M+H]⁺ 408.3/410.3.

### Intermediate D12: 5-Chloro-3,6-dimethyl-1-(tetrahydro-2H-pyran-2-yl)-4-(4,4,5,5-tetramethyl-1,3,2-dioxaborolan-2-yl)-1H-indazole

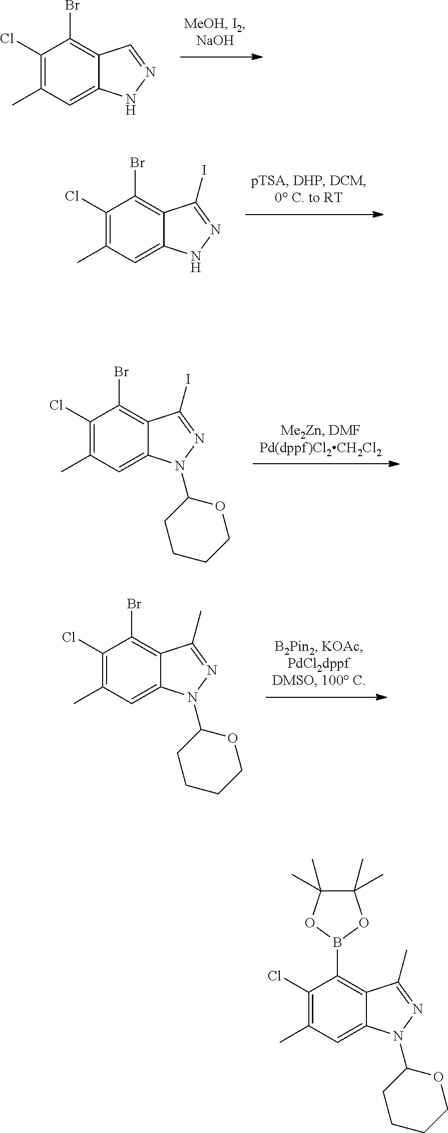 [SMILES: Cc1cc2[nH]ncc2c(Br)c1Cl ; Cc1cc2[nH]nc(I)c2c(Br)c1Cl ; Cc1cc2c(c(I)nn2C2CCCCO2)c(Br)c1Cl ; Cc1cc2c(c(C)nn2C2CCCCO2)c(Br)c1Cl ; Cc1cc2c(c(C)nn2C2CCCCO2)c(B2OC(C)(C)C(C)(C)O2)c1Cl]

### Step 1: 4-Bromo-5-chloro-3-iodo-6-methyl-1H-indazole

To a stirred solution of 4-bromo-5-chloro-6-methyl-1H-indazole (Intermediate D2, 20.0 g, 81.0 mmol) in MeOH (250 mL) was added NaOH (4 N, 132 mL, 530 mmol) and iodine (24.8 g, 98.0 mmol) at 0° C. and the reaction mixture was stirred at RT for 16 h. The RM was cooled down to 0° C. and acidified with HCl (4N), then Na₂S₂O₃ (10% solution) was added until a yellow suspension formed. EtOAc was added, the layers were separated (repeated twice) and the combined organic extracts were dried (Na₂SO₄), filtered and evaporated. The crude residue was purified by normal phase chromatography (eluent: EtOAc in c-hexane from 0 to 50%) to give the title compound as a yellow solid. UPLC-MS-4: Rt=1.23 min; MS m/z [M+H]⁺ 368.9/370.9/372.9.

### Step 2: 4-Bromo-5-chloro-3-iodo-6-methyl-1-(tetrahydro-2H-pyran-2-yl)-1H-indazole

To a suspension of 4-bromo-5-chloro-3-iodo-6-methyl-1H-indazole (Step 1, 23.7 g, 63.7 mmol) in CH₂Cl₂ (300 mL) was added under Ar p-toluenesulfonic acid monohydrate (0.61 g, 3.19 mmol) and dihydropyran (11.6 mL, 127 mmol) and the reaction mixture was stirred for RT for 2 h. The RM was quenched by addition of a sat. aq. NaHCO₃ solution, extracted with CH₂Cl₂ (×2) and the combined organic extracts were washed with a sat. aq. NaHCO₃ solution, dried (Na₂SO₄), filtered and evaporated. The crude residue was triturated in Et₂O and the precipitate was filtrated to give the title as a yellow solid. UPLC-MS-4: Rt=1.57 min; MS m/z [M+H]⁺ 455.1/457.1/459.1.

### Step 3: 4-Bromo-5-chloro-3,6-dimethyl-1-(tetrahydro-2H-pyran-2-yl)-1H-indazole

To a stirred solution of 4-bromo-5-chloro-3-iodo-6-methyl-1-(tetrahydro-2H-pyran-2-yl)-1H-indazole (Step 2, 12.4 g, 27.3 mmol) and PdCl₂(dppf). CH₂Cl₂ adduct (1.11 g, 1.36 mmol) in DMF (100 mL) was added under Ar Me₂Zn (2 M in toluene, 15.7 mL, 31.4 mmol) and the reaction mixture was stirred at 80° C. for 16 h. The RM was quenched with a sat. aq. NaHCO₃ solution, extracted with EtOAc (×2) and the combined organic extracts were washed with a sat. aq. NaHCO₃ solution, dried (Na₂SO₄), filtered and evaporated. The crude residue was purified by normal phase chromatography (eluent: EtOAc in c-hexane from 0 to 9%) to give the title compound as a yellow solid. UPLC-MS-4: Rt=1.48 min; MS m/z [M+H]⁺ 343.1/345.1/347.1.

### Step 4: 5-Chloro-3,6-dimethyl-1-(tetrahydro-2H-pyran-2-yl)-4-(4,4,5,5-tetramethyl-1,3,2-dioxaborolan-2-yl)-1H-indazole

A solution of 4-bromo-5-chloro-3,6-dimethyl-1-(tetrahydro-2H-pyran-2-yl)-1H-indazole (Step 3, 3.60 g, 10.5 mmol), bis(pinacolato)diboron (3.99 g, 15.7 mmol) and KOAc (3.08 g, 31.4 mmol) in DMSO (40 mL) was deoxygenated and refilled with argon (×3), then heated to 100° C. and PdCl₂(dppf). CH₂Cl₂ adduct (0.85 g, 1.05 mmol) was added. The reaction mixture was deoxygenated again (×3) and stirred for 16 h at 100° C. The RM was quenched with a sat. aq. NaHCO₃ solution, extracted with EtOAc (×2) and the combined organic extracts were washed with a sat. aq. NaHCO₃ solution, dried (Na₂SO₄), filtered and evaporated. The crude residue was purified by normal phase chromatography (eluent: EtOAc in c-hexane from 0 to 30%) to give the title compound as a white solid. UPLC-MS-4: Rt=1.38 min; MS m/z [M+H]⁺ 391.3/393.3.

### Intermediate D13: 5-chloro-1-(tetrahydro-2H-pyran-2-yl)-4-(4,4,5,5-tetramethyl-1,3,2-dioxaborolan-2-yl)-1H-pyrazolo[3,4-b]pyridine

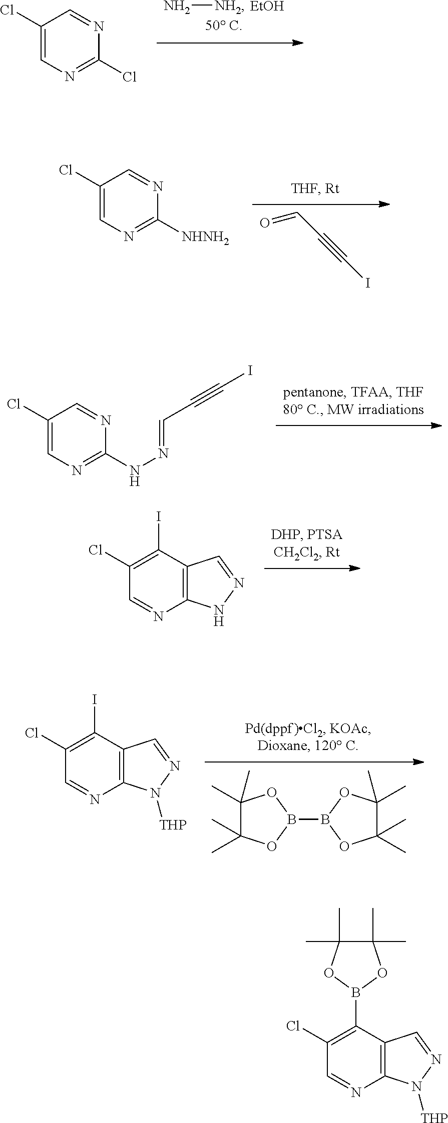

### Step 1: 5-Chloro-2-hydrazineylpyrimidine

In an Ace-tube, to a solution of 2,5-dichloropyrimidine (1.01 g, 6.78 mmol) in EtOH (7.5 mL) was added hydrazine monohydrate (0.66 mL, 13.6 mmol) and the resulting thick white suspension was vigorously stirred at 60° C. for 5.5 h. The precipitate was filtered-off, washed with EtOH and dried under HV. The white powder was taken up in EtOAc and a sat. aq. NaHCO₃ solution was added. The precipitate was filtered-off to give a first batch of the title compound. The 2 layers in the filtrate were separated and the aqueous layer was back extracted with EtOAc (×2). The combined organic extracts were dried (phase separator) and concentrated in vacuo to give a second batch of the title compound as a white solid. UPLC-MS-4: Rt=0.37 min; MS m/z [M+H]⁺ 145.0/147.0.

### Step 2: 5-Chloro-2-(2-(3-iodoprop-2-yn-1-ylidene)hydrazineyl)pyrimidine

In a microwave vial containing dry 3A° molecular sieves (810 mg) were added 5-chloro-2-hydrazineylpyrimidine (Step 1, 575 mg, 3.98 mmol), 3-iodoprop-2-ynal (CAS [20328-44-3], 753 mg, 3.98 mmol) and dry THF (15 mL) under argon. The vial was sealed and stirred at RT for 20 min. The reaction mixture was evaporated in vacuum to give the title compound as a mixture of cis/trans isomer as a brown solid which was used without further purification in the next step. UPLC-MS-4: Rt=0.67 and 0.80 min; MS m/z [M+H]⁺ 307.0/309.0.

### Step 3: 5-Chloro-4-iodo-1H-pyrazolo[3,4-b]pyridine

The title compound was prepared following procedures described in Le Fouler, V.; Chen, Y.; Gandon, V.; Bizet, V.; Salomé, C.; Fessard, T.; Liu, F.; Houk, K. N.; Blanchard, N. J. Am. Chem. Soc, 2019, 141, 15901. In a microwave vial under argon atmosphere, to a solution of 5-chloro-2-(2-(3-iodoprop-2-yn-1-ylidene)hydrazineyl)pyrimidine (Step 2, 1.26 g, 3.99 mmol) in dry THF (17 mL) were added 3-pentanone (1.27 mL, 12.0 mmol) and TFAA (0.84 mL, 5.98 mmol) dropwise. The vial was sealed and placed under microwave irradiations at 80° C. for 2 h. The reaction mixture was poured into an aq. sat. NaHCO₃ solution and extracted with EtOAc (×2). The combined organic extracts were dried (phase separator), concentrated in vacuo and the crude residue was purified by normal phase chromatography (eluent: MeOH in CH₂Cl₂ 0 to 3%) to give the title compound. UPLC-MS-4: Rt=1.23 min; MS m/z [M+H]⁺ 280.0/282.0.

### Step 4: 5-Chloro-4-iodo-1-(tetrahydro-2H-pyran-2-yl)-1H-pyrazolo[3,4-b]pyridine

To an ice-cooled suspension of 5-chloro-4-iodo-1H-pyrazolo[3,4-b]pyridine (Step 3, 690 mg, 2.35 mmol) in CH₂Cl₂ (5.50 mL) were added p-toluenesulfonic acid monohydrate (446 mg, 2.35 mmol) followed by 3,4-dihydro-2H-pyran (0.43 mL, 4.69 mmol). The reaction mixture was allowed to reach RT and stirred at RT for 23 h. p-Toluenesulfonic acid monohydrate (223 mg, 1.17 mmol) and 3,4-dihydro-2H-pyran (0.21 mL, 2.34 mmol) were added again and the reaction mixture was further stirred at RT for 16 h. The RM was poured into a sat. aq. solution of NaHCO₃ and extracted with CH₂Cl₂ (×3). The combined organic extracts were dried (phase separator), concentrated and the crude residue was purified by normal phase chromatography (eluent: EtOAc in n-heptane 0 to 40%) to give the title compound as a beige solid. UPLC-MS-4: Rt=1.23 min; MS m/z [M+H]⁺ 364.1/366.1.

### Step 5: 5-Chloro-1-(tetrahydro-2H-pyran-2-yl)-4-(4,4,5,5-tetramethyl-1,3,2-dioxaborolan-2-yl)-1H-pyrazolo[3,4-b]pyridine

A microwave vial was charged with 5-chloro-4-iodo-1-(tetrahydro-2H-pyran-2-yl)-1H-pyrazolo[3,4-b]pyridine (Step 4, 512 mg, 1.31 mmol), bis(pinacolato)diboron (665 mg, 2.62 mmol), PdCl₂(dppf)·CH₂Cl₂ adduct (107 mg, 0.13 mmol) and KOAc (386 mg, 3.93 mmol). 1,4-Dioxane (6.50 mL) was added and the mixture was degassed by sparging with argon and stirred at 120° C. (pre-heated oil bath) for 18 h. The reaction mixture was filtered through a pad of celite and washed with EtOAc. The filtrate was concentrated under reduced and the crude residue was purified by normal phase chromatography (eluent: MeOH in CH₂Cl₂ 0 to 5%) to give the title compound as a yellow oil. UPLC-MS-4: Rt=0.66 min; MS m/z [M−H]⁻ 280.2/282.2.

### Intermediate D14: 4-(4,4,5,5-tetramethyl-1,3,2-dioxaborolan-2-yl)naphthalen-2-ol

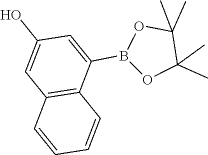 [SMILES: CC1(C)OB(c2cc(O)cc3ccccc23)OC1(C)C]

A mixture of 4-bromonaphtalen-2-ol (1.00 g, 4.48 mmol), bis(pinacolato)diboron (1.88 g, 7.40 mmol), KOAc (1.32 g, 13.45 mmol) and PdCl₂(dppf). CH₂Cl₂ adduct (0.37 g, 0.45 mmol) in dry dioxane (40 mL) under nitrogen was stirred at 90° C. for 2 h. The reaction mixture was diluted with EtOAc, filtered through a pad of celite and washed with EtOAc. The filtrate was concentrated and purified by normal phase chromatography (eluent: EtOAc in c-hexane from 0 to 50%) to give the title product as a beige solid. UPLC-MS-2a: Rt=1.16 min; MS m/z [M+H]⁺ 271.2.

### Intermediate D15: 2-(2-Chloro-5-(methoxymethoxy)-3,6-dimethylphenyl)-4,4,5,5-tetramethyl-1,3,2-dioxaborolane

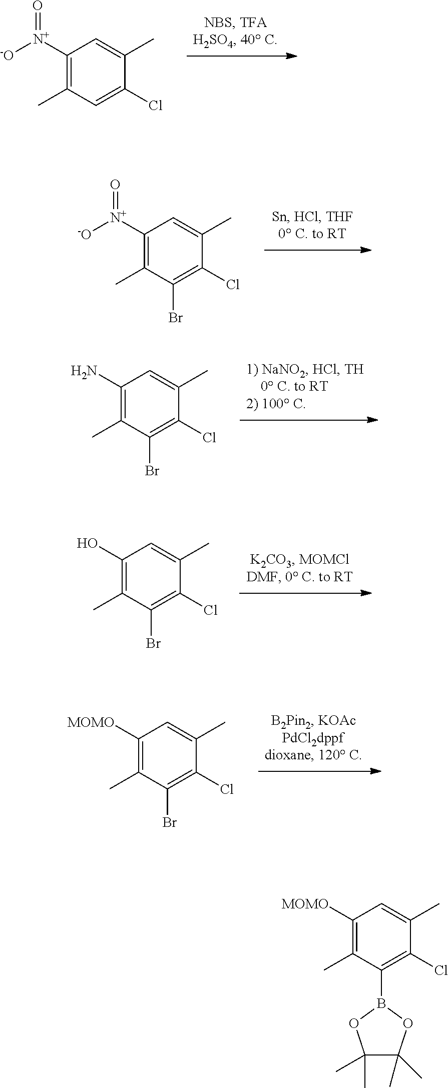

### Step 1: 3-Bromo-2-chloro-1,4-dimethyl-5-nitrobenzene

To a suspension of 1-chloro-2-5-dimethyl-4-nitrobenzene (24.9 g, 134 mmol) in TFA (75 mL) and conc. H₂SO₄ (25 mL) at 0° C. was added NBS (23.8 g, 134 mmol) portion wise under nitrogen atmosphere. The resulting solution was stirred at 40° C. for 5 h. After completion of the reaction, the RM was quenched with ice cold water and extracted with EtOAc. The combined organic extracts were washed with a sat. aq. NaHCO₃ solution, brine, dried (Na₂SO₄), filtered and concentrated under reduced pressure to give the title compound. ¹H NMR (400 MHz, CDCl₃) δ 7.66 (s, 1H), 2.57 (s, 3H), 2.34 (s, 3H).

### Step 2: 3-Bromo-4-chloro-2,5-dimethylaniline

To a solution of 3-bromo-2-chloro-1,4-dimethyl-5-nitrobenzene (33.0 g, 130 mmol) and Sn (46 g, 390 mmol) in THF (350 mL) cooled to 0° C. was added drop wise HCl (4M in water, 259 mL, 779.5 mmol) and the reaction mixture was stirred for 1 h at RT. After completion of the reaction, the RM was filtered through a celite pad and the filtrate was extracted with EtOAc. The combined organic extracts were washed with a sat. aq. NaHCO₃ solution, brine, dried (Na₂SO₄), filtered and concentrated under reduced pressure. The crude residue was purified by normal phase chromatography (eluent: 12-16% EtOAc in hexane) to give the title compound as pale brown solid flakes. ¹H NMR (400 MHz, DMSO-d₆) δ 6.60 (s, 1H), 5.24 (s, 2H), 2.27 (s, 3H), 2.23 (s, 3H).

### Step 3: 3-Bromo-4-chloro-2,5-dimethylphenol

To a solution of 3-bromo-4-chloro-2,5-dimethylaniline (10 g, 435 mmol) in THF (50 mL) at 0° C. was added drop wise HCl (6.0 N in water, 100 mL) and the mixture was stirred for 10 min. NaNO₂ (3.53 g, 522 mmol) in solution in a minimum amount of water was added drop wise into the above reaction mixture at 0° C., while maintaining the temperature at 0° C. and the mixture was stirred for 15 min and then 30 min at RT. This RM was added into another flask containing water (300 mL) and CPME (342 mL) with continuous stirring at RT and the reaction mixture was refluxed (100° C.) for 20 min. After completion of the reaction, the RM was allowed to cool to RT, was quenched with cold water and extracted by EtOAc. The combined organic extracts were washed with brine, dried (Na₂SO₄), filtered and concentrated under reduced pressure. The crude residue was purified by normal phase chormatography on neutral alumina oxide (eluent: 1.5-3.5% EtOAc in Hexane) to give the title compound as a light brown solid. ¹H NMR (400 MHz, DMSO-d₆) δ 9.95 (s, 1H), 6.80 (s, 1H), 2.33 (s, 3H), 2.29 (s, 3H).

### Step 4: 3-Bromo-2-chloro-5-(methoxymethoxy)-1,4-dimethylbenzene

3-Bromo-4-chloro-2,5-dimethylphenol (5.00 g, 21.5 mmol) was dissolved in DMF (50 mL). K₂CO₃ (8.88 g, 64.437 mmol) was added at 0° C. followed MOM-Cl (3.46 g, 42.9 mmol) drop wise under N₂ atmosphere and the reaction mixture was stirred at RT for 3 h. After completion of the reaction, the RM was quenched with water and extracted by EtOAc. The combined organic layers were washed with a sat. aq. NaHCO₃ solution, brine, dried (Na₂SO₄), filtered and concentrated under reduced pressure. The crude residue was purified normal phase chromatography on neutral alumina oxide (eluent: 1-1.8% EtOAc in Hexane) to give the title compound as a brown oil. ¹H NMR (400 MHz, CDCl₃) δ 6.97 (s, 1H), 5.19 (s, 2H), 3.51 (s, 3H), 2.42 (s, 3H), 2.34 (s, 3H).

### Step 5: 2-(2-Chloro-5-(methoxymethoxy)-3,6-dimethylphenyl)-4,4,5,5-tetramethyl-1,3,2-dioxaborolane

3-Bromo-2-chloro-5-(methoxymethoxy)-1,4-dimethylbenzene (2.04 g, 7.34 mmol), B₂Pin₂ (3.72 g, 14.7 mmol), KOAc (2.15 g, 22.0 mmol) and PdCl₂dppf (0.267 g, 0.37 mmol) were added to pre-degassed 1,4-dioxane (40 mL) at RT. The reaction mixture was stirred at 120° C. for 2 h in a sealed tube. After completion of the reaction, the reaction mixture was diluted with EtOAc, filtered through a celite pad and the filtrate was concentrated under reduce pressure. The crude residue was purified by normal phase chromatography (100% Hexane) to give the title compound as a pale yellow oil. ¹H NMR (400 MHz, CDCl₃) δ 6.97-6.93 (m, 1H), 5.15 (s, 2H), 3.50 (s, 3H), 2.38 (s, 3H), 2.30 (s, 3H), 1.44 (s, 12H).

### Intermediate D16: 5-Chloro-1,6-dimethyl-4-(4,4,5,5-tetramethyl-1,3,2-dioxaborolan-2-yl)-1H-indazole

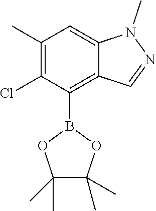 [SMILES: Cc1cc2c(cnn2C)c(B2OC(C)(C)C(C)(C)O2)c1Cl]

### Step 1: 4-Bromo-5-chloro-1,6-dimethyl-1H-indazole

To a solution of 4-bromo-5-chloro-6-methyl-1H-indazole (Intermediate D2, 2.00 g, 8.15 mmol) in DMF (27.2 mL) at 0° C. were added Cs₂CO₃ (5.31 g, 16.3 mmol) and MeI (0.51 mL, 8.15 mmol). The reaction mixture was stirred and warm to RT for 2 h. More MeI (0.051 mL, 0.815 mmol) was added to the reaction mixture to complete the reaction. The reaction mixture was quenched by addition of water and extracted with EtOAc (×2). The organic phase was washed with brine, dried (MgSO₄), filtered and concentrated in vacuo. The crude residue was purified by normal phase chromatography (eluent: EtOAc in heptane 0 to 70%) to give the title compound as the first eluting regiosiomer. UPLC-MS-4: Rt=1.25 min; MS m/z [M+H]⁺ 259.0/261.0.

### Step 2: 5-Chloro-1,6-dimethyl-4-(4,4,5,5-tetramethyl-1,3,2-dioxaborolan-2-yl)-1H-indazole

To a solution of 4-bromo-5-chloro-1,6-dimethyl-1H-indazole (step 1, 1.27 g, 4.89 mmol) in 1,4-dioxane (12.2 mL) were added B₂Pin₂ (2.48 g, 9.79 mmol), PdCl₂(dppf)·CH₂Cl₂ adduct (0.40 g, 0.49 mmol) and KOAc (1.44 g, 14.7 mmol). The reaction mixture was stirred at 110° C. for 5 h. The RM was quenched by addition of water. The solution was then extracted with CH₂Cl₂. The organic phase was washed with brine, dried (MgSO₄), filtered and concentrated in vacuo. The crude residue was purified by normal phase chromatography (eluent: EtOAc in heptane 0 to 10%) to give the title compound. UPLC-MS-4: Rt=1.26 min; MS m/z [M+H]⁺ 307.2/309.3.

### Intermediate D17: Tert-butyl 3-amino-5,6-dichloro-4-(4,4,5,5-tetramethyl-1,3,2-dioxaborolan-2-yl)-1H-indazole-1-carboxylate

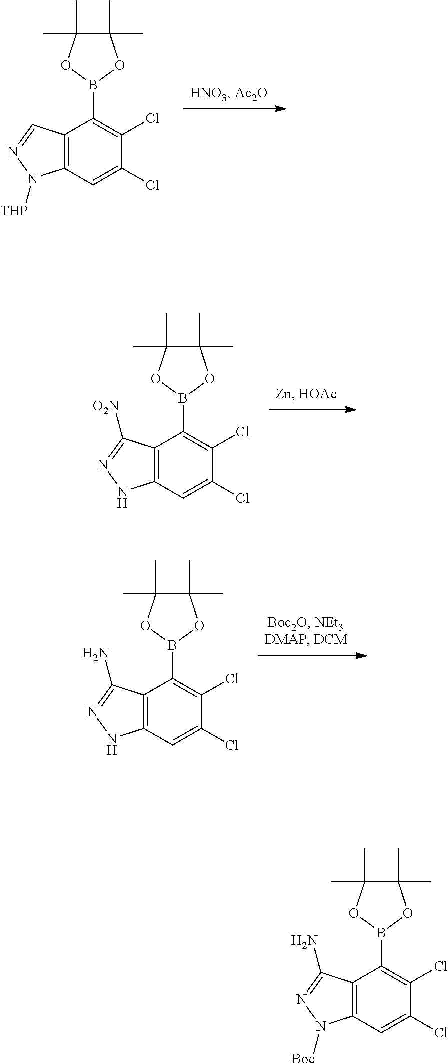

### Step 1: 5,6-Dichloro-3-nitro-4-(4,4,5,5-tetramethyl-1,3,2-dioxaborolan-2-yl)-1H-indazole

Fuming nitric acid (50% in TFA, 4.50 mL, 50.4 mmol) was slowly added to a solution of Intermediate D6, 5.00 g, 12.6 mmol) in HOAc (25 mL) at RT. Subsequently, acetic anhydride (2.40 mL, 25.6 mmol) was slowly added via a dropping funnel and the exothermic mixture was maintained at 60° C. After cooling slightly, the resulting suspension was quenched with ice-H₂O, sonicated and filtered to provide the title compound as a pale yellow solid. ¹H NMR (600 MHz, DMSO-d₆) δ 14.8 (br s, 1H), 8.19 (s, 1H), 1.42 (s, 12H); UPLC-MS-2e: Rt=5.95 min; MS m/z [M−H]⁻ 356.1/358.2/360.1.

### Step 2: 5,6-Dichloro-4-(4,4,5,5-tetramethyl-1,3,2-dioxaborolan-2-yl)-1H-indazol-3-amine

A mixture of 5,6-dichloro-3-nitro-4-(4,4,5,5-tetramethyl-1,3,2-dioxaborolan-2-yl)-1H-indazole (Step 1, 3.67 g, 9.30 mmol), Zn powder (6.10 g, 93.0 mmol) in HOAc (50 mL) was stirred at RT for 30 min. After filtering and washing the precipitate with EtOAc, the filtrate was concentrated under reduced pressure. The residue was diluted with a sat. aq. NaHCO₃ solution and extracted with EtOAc (2×). The combined organic layers were washed with brine, dried (phase separator) and concentrated under reduced pressure. The crude residue was purified by normal phase chromatography (eluent: MeOH in CH₂Cl₂ from 0 to 10% in 50 min) to provide the title compound. ¹H NMR (400 MHz, DMSO-d₆) δ 11.9 (br s, 1H), 7.62 (s, 1H), 4.99 (br s, 2H), 1.39 (s, 12H); UPLC-MS-4: Rt=0.45 min; MS m/z [M+H]⁺ 246.1/248.1/250.1 (hydrolysis to boronic acid under UPLC conditions).

### Step 3: Tert-Butyl 3-amino-5,6-dichloro-4-(4,4,5,5-tetramethyl-1,3,2-dioxaborolan-2-yl)-1H-indazole-1-carboxylate

DMAP (0.26 g, 2.14 mmol) was added to a solution of 5,6-dichloro-4-(4,4,5,5-tetramethyl-1,3,2-dioxaborolan-2-yl)-1H-indazol-3-amine (Step 2, 3.19 g, 8.56 mmol), Et₃N (2.39 mL, 17.12 mmol) and Boc-anhydride (1.96 g, 8.99 mmol) in CH₂Cl₂ (40 mL). After stirring overnight, the reaction mixture was diluted with CH₂Cl₂, washed with a sat. aq. NaHCO₃ solution, dried (phase separator) and concentrated under reduced pressure. The crude residue was purified by normal phase chromatography (eluent: MeOH in CH₂Cl₂ from 0 to 10% in 30 min) to provide the title compound. ¹H NMR (400 MHz, DMSO-d₆) δ 8.25 (s, 1H), 5.86 (s, 2H), 1.59 (s, 9H), 1.40 (s, 12H); UPLC-MS-4: Rt=1.02 min; MS m/z [M+H]⁻ 344.0/345.9/348.0 (hydrolysis to boronic acid under UPLC conditions).

### Intermediate D18: 5-Methyl-4-(4,4,5,5-tetramethyl-1,3,2-dioxaborolan-2-yl)-1-tosyl-1H-indazole

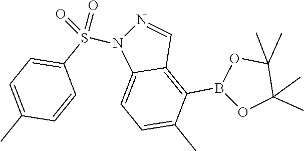

### Step 1: 4-Bromo-5-methyl-1-tosyl-1H-indazole

To an ice-cooled solution of 4-bromo-5-methyl-1H-indazole (5.00 g, 23.7 mmol) in THF (50 mL) under inert atmosphere was added NaH (1.90 g, 47.4 mmol), followed by toluene-4-sulfonyl chloride (4.97 g, 26.1 mmol) and the reaction mixture was stirred at RT for 1 h. The RM was quenched carefully at 0° C. with water and extracted with CH₂Cl₂. The organic phase was dried (Na₂SO₄), filtered and evaporated. The crude residue was triturated with Et₂O and the white precipitate was filtered, washed with cold Et₂O and dried under high vacuum to give the title compound as a white solid. UPLC-MS-1a: Rt=1.34 min; MS m/z [M+H]⁺ 365/367.

### Step 2: 5-Methyl-4-(4,4,5,5-tetramethyl-1,3,2-dioxaborolan-2-yl)-1-tosyl-1H-indazole

To a solution of 4-bromo-5-methyl-1-tosyl-1H-indazole (step 1, 7.94 g, 21.7 mmol) in 1,4-dioxane (80 mL) under inert atmosphere were added B₂Pin₂ (11.0 g, 43.5 mmol), PdCl₂(dppf)·CH₂Cl₂ adduct (0.89 g, 1.09 mmol) and KOAc (6.40 g, 65.2 mmol). The reaction mixture was degassed then stirred at 100° C. for 16 h. The RM was quenched with water and extracted with CH₂Cl₂. The organic phase was washed with brine, then dried (Na₂SO₄, filtered and evaporated. The crude residue was triturated with Et₂O, the white precipitate was filtered, washed with cold Et₂O and dried under high vacuum. The crude product was purified by normal phase chromatography (eluent: EtOAc in c-hexane from 0 to 40%) to give the title compound. UPLC-MS-1a: Rt=1.48 min; MS m/z [M+H]⁺ 413.

### Biological Assays and Biological Data

The activity of a compound according to the present invention can be assessed by the following in vitro methods.

### Purification of Human KRasG12C 1-169, Biotinylated on the N-Terminus

Human KRASG12C (UniProtKB: P01116) amino acids M1-K169 were expressed from plasmid pCZ239 (SEQ ID NO: 1) in E. coli BL21 (DE3) in the presence of a plasmid encoding biotin-[acetyl-CoA-carboxylase] ligase BirA (NCBI Reference Sequence: NP_418404.1, aa 1-321, full length) in Luria-Bertani (LB) medium-supplemented with 25 μg/ml kanamycin, 34 μg/ml chloramphenicol, 135 □M biotin and 1 mM isopropyl β-D-1-thiogalactopyranoside overnight at 18° C. Cells were harvested by centrifugation and frozen at −80° C. until further processing. Cells were thawed and resuspended in Buffer A (20 mM Tris-HCl pH8, 500 mM NaCl, 5 mM imidazole, 2 mM TCEP, 10% glycerol) supplemented with 1 tablet protease inhibitor/50 ml buffer (Complete EDTA free, Roche) and 15 μl Turbonuclease (90% purity, 50KUN, ≥200,000 units/mL, Sigma) and incubated for 20 min at 4° C. The cells were lysed by 4 passages through an Avestin Emulsiflex at about 20000 psi and insoluble debris removed by centrifugation and filtered through a 0.45 □m Durapore membrane (Millipore). Immobilized metal affinity chromatography was performed with a HisTrap HP 5 mL column (GE) with Buffer A and eluted with a linear gradient over 10 column volumes of Buffer A substituted with 200 mM imidazole. Eluted protein fractions were analyzed by Novex NuPage 4-12% PAGE. The affinity tag was removed by HRV3C protease cleavage (HRV3C protease produced in-house but also available commercially) during dialysis against Buffer A for 18 h at 4° C. and capturing by a reverse IMAC purification on a HisTrap HP 5 mL column. The protein in the flow through was subjected to a final polishing step on a HiLoad 16/60 Superdex 200 prep grade size-exclusion column pre-equilibrated with SEC-buffer (20 mM HEPES pH 7.4, 150 mM NaCl, 5 mM MgCl₂, 1 □M GDP). Positive fractions were determined by PAGE (Novex NuPage 4-12% BisTris) analysis. The correct mass was determined by RP (Reverse Phase) LC-MS, and indicated complete biotinylation of the protein.

DNA sequence of plasmid pCZ239, coding
sequence underlined
SEQ ID No: 1
ATGCGTCCGGCGTAGAGGATCGAGATCGATCTCGATCCCGCGAAA
TTAATACGACTCACTATAGGGGAATTGTGAGCGGATAACAATTCC
CCTCTAGAAATAATTTTGTTTAACTTTAAGAAGGAGATATACATA
TGAAAACACATCATCATCATCATCATGGTGGCGGCAGTGGCGGTG
GCTCAGGCGGTGGTTCTCTCGAGGTTCTGTTCCAGGGTCCGGGTT
TGAACGACATCTTCGAAGCTCAGAAGATCGAATGGCACGAGGGTG
GCGGTAGTGGTGGTGGCTCTATGACTGAATACAAGCTGGTTGTTG
TTGGTGCTTGTGGCGTTGGTAAGAGCGCACTGACCATCCAGCTCA
TTCAGAATCACTTCGTGGACGAGTACGACCCGACCATCGAAGATT
CTTACCGTAAACAGGTGGTTATTGATGGCGAAACCTGTCTGCTGG
ATATTCTGGACACTGCTGGTCAGGAAGAGTACTCCGCTATGCGTG
ATCAGTACATGCGTACTGGTGAAGGTTTCCTCTGCGTGTTCGCTA
TCAACAACACCAAGTCCTTCGAAGATATCCACCATTACCGTGAAC
AGATCAAACGTGTGAAGGACAGCGAAGACGTGCCAATGGTTCTGG
TGGGCAACAAATGTGATCTCCCGAGCCGTACCGTTGACACCAAAC
AGGCACAAGACCTGGCACGTTCCTACGGCATCCCATTCATTGAAA
CTAGCGCGAAGACTCGTCAGGGTGTGGACGACGCATTCTACACTC
TGGTGCGTGAAATTCGCAAGCACAAAGAGAAATAATGGTACCGAA
TTCGCGGCCGCCTGCAGCCTAGGCTGCTAAACAAAGCCCGAAAGG
AAGCTGAGTTGGCTGCTGCCACCGCTGAGCAATAACTAGCATAAC
CCCTTGGGGCCTCTAAACGGGTCTIGAGGGGTTTTTTGCTGAAAG
GAGGAACTATATCCGGATTGGCGAATGGGACGCGCCCTGTAGCGG
CGCATTAAGTGCAGCGTCAAAAGGGCGACACAAAATTTATTCTAA
ATGCATAATAAATACTGATAACATCTTATAGTTTGTATTATATTT
TGTATTATCGTTGACATGTATAATTTTGATATCAAAAACTGATTT
TCCCTTTATTATTTTCGAGATTTATTTTCTTAATTCTCTTTAACA
AACTAGAAATATTGTATATACAAAAAATCATAAATAATAGATGAA
TAGTTTAATTATAGGTGTTCATCAATCGAAAAAGCAACGTATCTT
ATTTAAAGTGCGTTGCTTTTTTCTCATTTATAAGGTTAAATAATT
CTCATATATCAAGCAAAGTGACAGGCGCCCTTAAATATTCTGACA
AATGCTCTTTCCCTAAACTCCCCCCATAAAAAAACCCGCCGAAGC
GGGTTTTTACGTTATTTGCGGATTAACGATTACTCGTTATCAGAA
CCGCCCAGGGGGCCCGAGCTTAAGACTGGCCGTCGTTTTACAACA
CAGAAAGAGTTTGTAGAAACGCAAAAAGGCCATCCGTCAGGGGCC
TTCTGCTTAGTTTGATGCCTGGCAGTTCCCTACTCTCGCCTTCCG
CTTCCTCGCTCACTGACTCGCTGCGCTCGGTCGTTCGGCTGCGGC
GAGCGGTATCAGCTCACTCAAAGGCGGTAATACGGTTATCCACAG
AATCAGGGGATAACGCAGGAAAGAACATGTGAGCAAAAGGCCAGC
AAAAGGCCAGGAACCGTAAAAAGGCCGCGTTGCTGGCGTTTTTCC
ATAGGCTCCGCCCCCCTGACGAGCATCACAAAAATCGACGCTCAA
GTCAGAGGTGGCGAAACCCGACAGGACTATAAAGATACCAGGCGT
TTCCCCCTGGAAGCTCCCTCGTGCGCTCTCCTGTTCCGACCCTGC
CGCTTACCGGATACCTGTCCGCCTTTCTCCCTTCGGGAAGCGTGG
CGCTTTCTCATAGCTCACGCTGTAGGTATCTCAGTTCGGTGTAGG
TCGTTCGCTCCAAGCTGGGCTGTGTGCACGAACCCCCCGTTCAGC
CCGACCGCTGCGCCTTATCCGGTAACTATCGTCTTGAGTCCAACC
CGGTAAGACACGACTTATCGCCACTGGCAGCAGCCACTGGTAACA
GGATTAGCAGAGCGAGGTATGTAGGCGGTGCTACAGAGTTCTTGA
AGTGGTGGGCTAACTACGGCTACACTAGAAGAACAGTATTTGGTA
TCTGCGCTCTGCTGAAGCCAGTTACCTTCGGAAAAAGAGTTGGTA
GCTCTTGATCCGGCAAACAAACCACCGCTGGTAGCGGTGGTTTTT
TTGTTTGCAAGCAGCAGATTACGCGCAGAAAAAAAGGATCTCAAG
AAGATCCTTTGATCTTTTCTACGGGGTCTGACGCTCAGTGGAACG
ACGCGCGCGTAACTCACGTTAAGGGATTTTGGTCATGAGCTTGCG
CCGTCCCGTCAAGTCAGCGTAATGCTCTGCTTTTAGAAAAACTCA
TCGAGCATCAAATGAAACTGCAATTTATTCATATCAGGATTATCA
ATACCATATTTTTGAAAAAGCCGTTTCTGTAATGAAGGAGAAAAC
TCACCGAGGCAGTTCCATAGGATGGCAAGATCCTGGTATCGGTCT
GCGATTCCGACTCGTCCAACATCAATACAACCTATTAATTTCCCC
TCGTCAAAAATAAGGTTATCAAGTGAGAAATCACCATGAGTGACG
ACTGAATCCGGTGAGAATGGCAAAAGTTTATGCATTTCTTTCCAG
ACTTGTTCAACAGGCCAGCCATTACGCTCGTCATCAAAATCACTC
GCATCAACCAAACCGTTATTCATTCGTGATTGCGCCTGAGCGAGG
CGAAATACGCGATCGCTGTTAAAAGGACAATTACAAACAGGAATC
GAGTGCAACCGGCGCAGGAACACTGCCAGCGCATCAACAATATTT
TCACCTGAATCAGGATATTCTTCTAATACCTGGAACGCTGTTTTT
CCGGGGATCGCAGTGGTGAGTAACCATGCATCATCAGGAGTACGG
ATAAAATGCTTGATGGTCGGAAGTGGCATAAATTCCGTCAGCCAG
TTTAGTCTGACCATCTCATCTGTAACATCATTGGCAACGCTACCT
TTGCCATGTTTCAGAAACAACTCTGGCGCATCGGGCTTCCCATAC
AAGCGATAGATTGTCGCACCTGATTGCCCGACATTATCGCGAGCC
CATTTATACCCATATAAATCAGCATCCATGTTGGAATTTAATCGC
GGCCTCGACGTTTCCCGTTGAATATGGCTCATATTCTTCCTTTTT
CAATATTATTGAAGCATTTATCAGGGTTATTGTCTCATGAGCGGA
TACATATTTGAATGTATTTAGAAAAATAAACAAATAGGGGTCAGT
GTTACAACCAATTAACCAATTCTGAACATTATCGCGAGCCCATTT
ATACCTGAATATGGCTCATAACACCCCTTGTTTGCCTGGCGGCAG
TAGCGCGGTGGTCCCACCTGACCCCATGCCGAACTCAGAAGTGAA
ACGCCGTAGCGCCGATGGTAGTGTGGGGACTCCCCATGCGAGAGT
AGGGAACTGCCAGGCATCAAATAAAACGAAAGGCTCAGTCGAAAG
ACTGGGCCTTTCGCCCGGGCTAATTAGGGGGTGTCGCCCTTCGCT
GAAGAATTGATCCCGGTGCCTAATGAGTGAGCTAACTTACATTAA
TTGCGTTGCGCTCACTGCCCGCTTTCCAGTCGGGAAACCTGTCGT
GCCAGCTGCATTAATGAATCGGCCAACGCGCGGGGAGAGGCGGTT
TGCGTATTGGGCGCCAGGGTGGTTTTTCTTTTCACCAGTGACACG
GGCAACAGCTGATTGCCCTTCACCGCCTGGCCCTGAGAGAGTTGC
AGCAAGCGGTCCACGCTGGTTTGCCCCAGCAGGCGAAAATCCTGT
TTGATGGTGGTTAACGGCGGGATATAACATGAGCTGTCTTCGGTA
TCGTCGTATCCCACTACCGAGATGTCCGCACCAACGCGCAGCCCG
GACTCGGTAATGGCGCGCATTGCGCCCAGCGCCATCTGATCGTTG
GCAACCAGCATCGCAGTGGGAACGATGCCCTCATTCAGCATTTGC
ATGGTTTGTTGAAAACCGGACATGGCACTCCAGTCGCCTTCCCGT
TCCGCTATCGGCTGAATTTGATTGCGAGTGAGATATTTATGCCAG
CCAGCCAGACGCAGACGCGCCGAGACAGAACTTAATGGGCCCGCT
AACAGCGCGATTTGCTGGTGACCCAATGCGACCAGATGCTCCACG
CCCAGTCGCGTACCGTCTTCATGGGAGAAAATAATACTGTTGATG
GGTGTCTGGTCAGAGACATCAAGAAATAACGCCGGAACATTAGTG
CAGGCAGCTTCCACAGCAATGGCATCCTGGTCATCCAGCGGATAG
TTAATGATCAGCCCACTGACGCGTTGCGCGAGAAGATTGTGCACC
GCCGCTTTACAGGCTTCGACGCCGCTTCGTTCTACCATCGACACC
ACCACGCTGGCACCCAGTTGATCGGCGCGAGATTTAATCGCCGCG
ACAATTTGCGACGGCGCGTGCAGGGCCAGACTGGAGGTGGCAACG
CCAATCAGCAACGACTGTTTGCCCGCCAGTTGTTGTGCCACGCGG
TTGGGAATGTAATTCAGCTCCGCCATCGCCGCTTCCACTTTTTCC
CGCGTTTTCGCAGAAACGTGGCTGGCCTGGTTCACCACGCGGGAA
ACGGTCTGATAAGAGACACCGGCATACTCTGCGACATCGTATAAC
GTTACTGGTTTCACATTCACCACCCTGAATTGACTCTCTTCCGGG
CGCTATCATGCCATACCGCGAAAGGTTTTGCGCCATTCGATGGTG
TCCGGGATCTCGACGCTCTCCCTTATGCGACTCCTGCATTAGGAA
GCAGCCCAGTAGTAGGTTGAGGCCGTTGAGCACCGCCGCCGCAAG
GAATGGTGCATGCAAGGAGATGGCGCCCAACAGTCCCCCGGCCAC
GGGGCCTGCCACCATACCCACGCCGAAACAAGCGCTCATGAGCCC
GAAGTGGCGAGCCCGATCTTCCCCATCGGTGATGTCGGCGATATA
GGCGCCAGCAACCGCACCTGTGGCGCCGGTGATGCCGGCCACG

### Preparation of: N-(3-fluoro-4-(2-methyl-3-(5-methyl-1H-indazol-4-yl)-1H-pyrrolo[2,3-b]pyridin-1-yl)phenyl)acrylamide (Compound A)

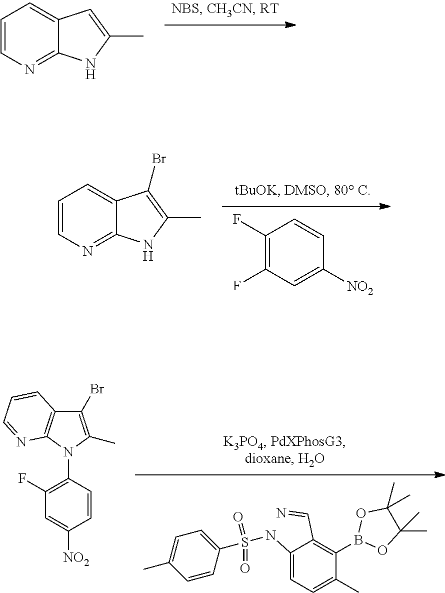

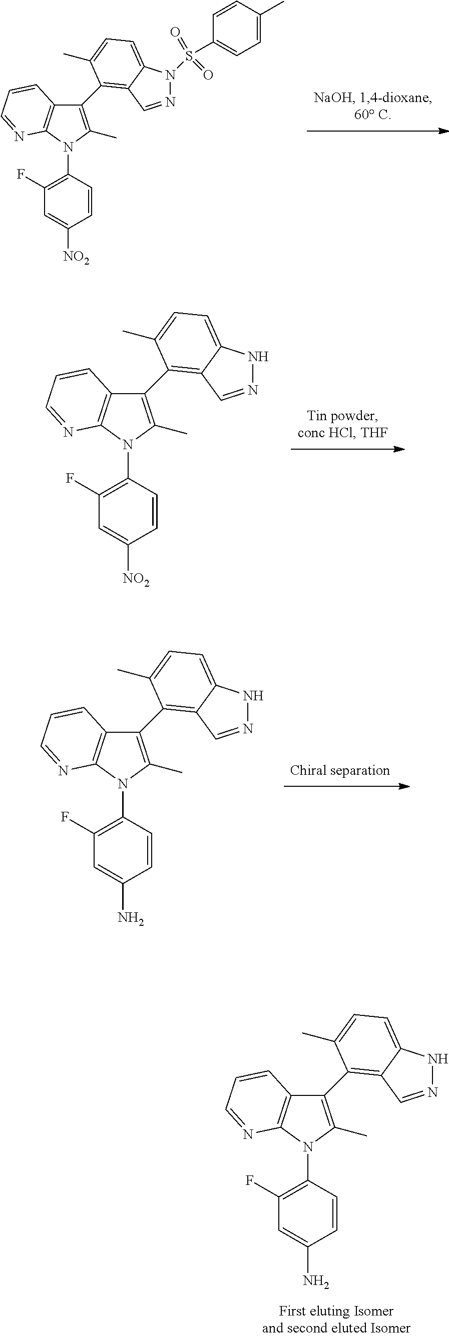 [SMILES: Cc1ccc(S(=O)(=O)n2ncc3c(-c4c(C)n(-c5ccc([N+](=O)[O-])cc5F)c5ncccc45)c(C)ccc32)cc1 ; Cc1ccc2[nH]ncc2c1-c1c(C)n(-c2ccc([N+](=O)[O-])cc2F)c2ncccc12 ; Cc1ccc2[nH]ncc2c1-c1c(C)n(-c2ccc(N)cc2F)c2ncccc12 ; Cc1ccc2[nH]ncc2c1-c1c(C)n(-c2ccc(N)cc2F)c2ncccc12]

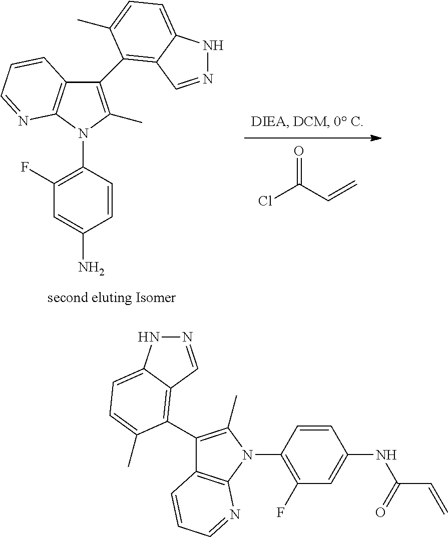

### Step 1: 3-Bromo-2-methyl-1H-pyrrolo[2,3-b]pyridine

To a solution of 2-methyl-1H-pyrrolo[2,3-b]pyridine (1.63 g, 12.3 mmol) in acetonitrile (50 mL) was added NBS (2.19 g, 12.3 mmol) at RT. The reaction mixture was stirred at RT for 1 h. The reaction was quenched by addition of a 10% solution of sodium thiosulfate (10 mL). The solution was then extracted with EtOAc (20 mL). The organic phase was washed with water (3×), brine, then dried (MgSO₄), filtered and evaporated. UPLC-MS-1: Rt=0.88 min; MS m/z [M+H]⁺ 211/213.

### Step 2: 3-Bromo-1-(2-fluoro-4-nitrophenyl)-2-methyl-1H-pyrrolo[2,3-b]pyridine

To a solution of 3-bromo-2-methyl-1H-pyrrolo[2,3-b]pyridine (Step 1, 2.4 g, 11.4 mmol) in dry DMSO (20 mL) was added under inert atmosphere potassium tert-butoxide (1.40 g, 12.5 mmol) and 1,2-difluoro-4-nitrobenzene (1.81 g, 11.4 mmol). The reaction mixture was stirred at 80° C. for 2 h. The reaction was quenched by addition of water. The solution was then extracted with EtOAc (3×). The organic phase was washed with brine, then dried (MgSO₄), filtered and evaporated. The crude product was purified by normal phase chromatography (eluent: EtOAc in Heptane 0 to 30%) to give the title compound. UPLC-MS-1: Rt=1.24 min; MS m/z [M+H]⁺ 350.0/352.1.

### Step 3: 4-(1-(2-Fluoro-4-nitrophenyl)-2-methyl-1H-pyrrolo[2,3-b]pyridin-3-yl)-5-methyl-1-tosyl-1H-indazole

To a solution of 3-bromo-1-(2-fluoro-4-nitrophenyl)-2-methyl-1H-pyrrolo[2,3-b]pyridine (Step 2, 1.00 g, 2.86 mmol) in 1,4-dioxane (10 mL)/water (2.8 mL) was added under inert atmosphere 5-methyl-4-(4,4,5,5-tetramethyl-1,3,2-dioxaborolan-2-yl)-1-tosyl-1H-indazole (Intermediate D18) (1.41 g, 3.43 mmol) followed by K₃PO₄ (1.21 g, 5.71 mmol) and then Pd-XPhos-G3 (0.24 g, 0.29 mmol). The reaction mixture was stirred 3 h at 80° C. The RM was quenched by addition of saturated solution of NaHCO₃. The solution was then extracted with EtOAc. The organic phase was washed with brine, then dried (MgSO₄), filtered and evaporated. The crude product was purified by normal phase chromatography (eluent: EtOAc in n-heptane 0 to 50%) to give the title compound. UPLC-MS-1: Rt=1.38 min; MS m/z [M+H]⁺ 556.2.

### Step 4: 4-(1-(2-Fluoro-4-nitrophenyl)-2-methyl-1H-pyrrolo[2,3-b]pyridin-3-yl)-5-methyl-1H-indazole

To a stirred solution of 4-(1-(2-fluoro-4-nitrophenyl)-2-methyl-1H-pyrrolo[2,3-b]pyridin-3-yl)-5-methyl-1-tosyl-1H-indazole (Step 3, 1.34 g, 2.41 mmol) in 1,4-dioxane (12 mL) was added NaOH (6.03 mL, 12.1 mmol) at RT. The reaction mixture was stirred at 60° C. for 3 h. The RM was quenched by addition of water. The solution was then extracted with EtOAc. The organic phase was washed with brine, dried over MgSO₄, filtered and evaporated. The crude product was purified by normal phase chromatography (eluent: EtOAc in Heptane 0 to 100%) to give the title compound. UPLC-MS-1: Rt=1.11 min; MS m/z [M+H]⁺ 402.2.

### Step 5: 3-Fluoro-4-(2-methyl-3-(5-methyl-1H-indazol-4-yl)-1H-pyrrolo[2,3-b]pyridin-1-yl)aniline

To a solution of 4-(1-(2-fluoro-4-nitrophenyl)-2-methyl-1H-pyrrolo[2,3-b]pyridin-3-yl)-5-methyl-1H-indazole (Step 4, 780 mg, 1.94 mmol) in THF (10 mL) were added Tin powder (807 mg, 6.80 mmol) and HCl conc (0.59 mL, 19.4 mmol). The solution was stirred at 70° C. for 4 h. The reaction was quenched by addition of sodium hydroxide and water. The solution was then extracted with EtOAc. The organic phase was washed with brine, dried over MgSO₄, filtered and evaporated to give the title compound. UPLC-MS-1: Rt=0.93 min; MS m/z [M+H]⁺ 372.4.

### Step 6: 3-Fluoro-4-(2-methyl-3-(5-methyl-1H-indazol-4-yl)-1H-pyrrolo[2,3-b]pyridin-1-yl)aniline

3-Fluoro-4-(2-methyl-3-(5-methyl-1H-indazol-4-yl)-1H-pyrrolo[2,3-b]pyridin-1-yl)aniline as mixture of isomers (454 mg) in IPA (30 mg/mL) were separated by chiral SFC (column: Lux IC 5 μm; 250×21.2 mm; mobile phase: CO₂/IPA 55/45; flow rate: 50 mL/min; column temperature: 40° C.; back pressure: 105 bars) to give 3-fluoro-4-(2-methyl-3-(5-methyl-1H-indazol-4-yl)-1H-pyrrolo[2,3-b]pyridin-1-yl)aniline as first eluting isomer (analytical chiral SFC; column: Chiralpak AD-H 5 μm; 250×4.6 mm; mobile phase: CO₂/[IPA+1% isopropylamine]: 50/50; flow rate: 3 mL/min; column temperature: 40° C.; back pressure: 120 bars): Rt=2.99 min and 3-fluoro-4-(2-methyl-3-(5-methyl-1H-indazol-4-yl)-1H-pyrrolo[2,3-b]pyridin-1-yl)aniline as the second eluting isomer (analytical chiral SFC; column: Chiralpak AD-H 5 μm; 250×4.6 mm; mobile phase: CO₂/[IPA+1% isopropylamine]: 50/50; flow rate: 3 mL/min; column temperature: 40° C.; back pressure: 120 bars): Rt=5.79 min. UPLC-MS-1: Rt=0.92 min; MS m/z [M+H]⁺ 372.2.

### Step 7: N-(3-Fluoro-4-(2-methyl-3-(5-methyl-1H-indazol-4-yl)-1H-pyrrolo[2,3-b]pyridin-1-yl)phenyl)acrylamide

To a solution of 3-fluoro-4-(2-methyl-3-(5-methyl-1H-indazol-4-yl)-1H-pyrrolo[2,3-b]pyridin-1-yl)aniline (Step 6, second eluting peak, 19 mg, 0.05 mmol) in CH₂Cl₂ (1.5 mL) were added DIPEA (0.03 mL, 0.15 mmol) and acryloyl chloride (4.57 μl, 0.06 mmol) at 0° C. The reaction mixture was stirred at 0° C. for 4 h. MeOH was added to the mixture and evaporated to dryness. The reaction was quenched by addition of saturated solution of NaHCO₃. The solution was then extracted with CH₂Cl₂. The organic phase was washed with brine, dried over MgSO₄, filtered and evaporated. The crude product was purified by normal phase chromatography (eluent: EtOAc in n-heptane 0 to 100%) to give the title compound. ¹H NMR (600 MHz, DMSO-d₆) δ 13.12-13.03 (m, 1H), 10.63 (s, 1H), 8.21-8.16 (m, 1H), 8.03-7.96 (m, 1H), 7.79-7.45 (m, 5H), 7.43-7.37 (m, 1H), 7.18-7.10 (m, 1H), 6.54-6.45 (m, 1H), 6.40-6.32 (m, 1H), 5.90-5.83 (m, 1H), 2.29-2.22 (m, 3H), 2.09 (s, 3H); UPLC-MS-1: Rt=0.98 min; MS m/z [M+H]⁺; 426.4.

### Preparation of [acrylamide-2,3-³H₂]—N-(3-fluoro-4-(2-methyl-3-(5-methyl-1H-indazol-4-yl)-1H-pyrrolo[2,3-b]pyridin-1-yl)phenyl)acrylamide (Compound B)

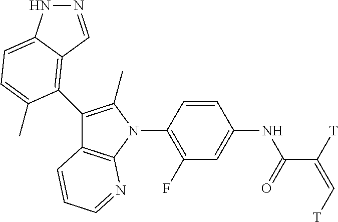

### Step 1: N-(3-Fluoro-4-(2-methyl-3-(5-methyl-1H-indazol-4-yl)-1H-pyrrolo[2,3-b]pyridin-1-yl)phenyl)propiolamide

To an ice-cooled solution of 3-fluoro-4-(2-methyl-3-(5-methyl-1H-indazol-4-yl)-1H-pyrrolo[2,3-b]pyridin-1-yl)aniline (Compound A, step 6 second eluting peak) (50 mg, 0.14 mmol) in DMF (1.5 mL) was added a mixture of DIPEA (0.09 mL, 0.54 mmol), propiolic acid (9.43 mg, 0.14 mmol) and propylphosphonic anhydride (50% in DMF, 0.16 mL, 0.27 mmol). The reaction mixture was stirred at RT under nitrogen for 15 min. The reaction mixture was poured into a sat. aq. NaHCO₃ solution and extracted with EtOAc (×3). The combined organic layers were dried over MgSO₄, and concentrated. The crude product was purified by achiral SFC (column: Princeton PPU 5 μm; 250×30 mm; mobile phase: CO₂/MeOH: gradient with 24-29% MeOH in CO₂ over 9.8 minutes; flow rate: 30 mL/min; column temperature: 36° C.; back pressure: 120 bars) and re-purified by achiral SFC (column: Princeton PPU 5 μm; 250×30 mm; mobile phase: CO₂/MeOH: gradient with 20-26% MeOH in CO₂ over 9.8 minutes; flow rate: 30 mL/min; column temperature: 36° C.; back pressure: 120 bars) to give the title compound. UPLC-MS-1: Rt=0.97 min; MS m/z [M+H]⁺ 424.4.

### Step 2: [Acrylamide-2,3-³H₂]—N-(3-fluoro-4-(2-methyl-3-(5-methyl-1H-indazol-4-yl)-1H-pyrrolo[2,3-b]pyridin-1-yl)phenyl)acrylamide

N-(3-Fluoro-4-(2-methyl-3-(5-methyl-1H-indazol-4-yl)-1H-pyrrolo[2,3-b]pyridin-1-yl)phenyl)propiolamide (step 1) (3.20 mg, 7.56 μmol), Lindlar catalyst (6.57 mg), quinoline (11.8 μL, 12.90 mg, 99.6 μmol) were suspended in DMF (0.60 mL). The suspension was degassed three times at the high vacuum manifold and stirred under an atmosphere of tritium gas (355 GBq, 508 mbar initial pressure) for 80 mins at room temperature (end pressure was 505 mbar, no more tritium gas consumption was observed). The solvent was removed in vacuo, and labile tritium was exchanged by adding methanol (0.70 mL), stirring the solution, and removing the solvent again under vacuo. This process was repeated two times. Finally, the well dried solid was extracted with 5 mL of ethanol and the suspension was filtered through a 0.2 μm nylon membrane obtaining a clear and colourless solution. The radiochemical purity was determined to 86% by HPLC (Waters Sunfire HPLC with UV detector; column: C18 5 μm; 250×4.6 mm; mobile phase: A: water/B: acetonitrile, 0 min 10% B, 10 min 95% B, 14.5 min 95% B, 15 min 10% B; flow rate: 1 mL/min; column temperature: 30° C.). Purification of the crude product was carried out by reverse phase HPLC (Waters Sunfire; column: C18 5 μm; 250×10 mm; detection UV 254 nM; mobile phase: A: water/B: MeOH, isocratic 62% B; flow rate: 4.7 mL/min; column temperature: 25° C.). The target compound eluted at 19.1 min. The combined HPLC fractions were partially reduced at the rotary evaporator at 40° C. Then, the product was extracted with a Phenomenex StrataX cartridge (33 μm Polymeric Reversed Phase, 100 mg, 3 mL; 8B-S100-EB) which was eluted with 5 mL of ethanol. The extracted product contained the title compound with an activity of 2.61 GBq and a radiochemical purity of >99%. The molar activity was determined to be 2.12 TBq/mmol.

### In Vitro Biochemical Quantification of Covalent Modification of KRASG12C

A scintillation proximity assay (SPA) was used to determine the potency of the compounds.

This assay measures the ability of the test compound to compete with the radiolabeled covalent probe for binding to and covalently modify KRASG12C.

The signal to be measured is generated by binding of an isotopic dilution of the covalent radio-ligand [[acrylamide-2,3-³H₂]—N-(3-fluoro-4-(2-methyl-3-(5-methyl-1H-indazol-4-yl)-1H-pyrrolo[2,3-b]pyridin-1-yl)phenyl)acrylamide (Compound B) (2.12 TBq/mmol) and its non-labeled analog [N-(3-fluoro-4-(2-methyl-3-(5-methyl-1H-indazol-4-yl)-1H-pyrrolo[2,3-b]pyridin-1-yl)phenyl)acrylamide (Compound A) to KRasG12C (M1-K169, biotinylated on the N-terminus) which is bound to SPA beads via biotin-streptavidin coupling. (see above for synthesis of Compound A and Compound B). A serial dilution of the compounds to be tested is mixed with a fixed concentration of the radiolabeled covalent probe before incubation with KRASG12C:GDP as described below. KRASG12C can either be bound by the radiolabeled covalent probe (Compound B), resulting in light being emitted from the beads, or bound by covalent test compound preventing signal generation.

Assays were run using 384-well plates (781207/Greiner) in which one column was designated as the high signal (no inhibition) control, and contained DMSO with no test compound, and another column was designated as the low signal control (maximal inhibition), and contained no protein. Serial dilutions of compounds to be tested were added to the assay plate (resulting in duplicate 11-point dose response with semi-log compound dilutions from 50 μM to 0.5 nM or from 5 μM to 0.05 nM for the most potent compounds). A 1/20 isotopic dilution of labeled (Compound B) and non-labeled covalent (Compound A) probe was prepared and added to all wells on the plate. The reaction was started by addition of KRasG12C (M1-K169, biotinylated on the N-terminus) to the compounds and incubated for 2 hours with continuous agitation allowing for full modification of KRasG12C with probe or test compounds. Final concentrations in an assay volume of 40 μL were 10 nM KRasG12C, 25 nM radio-ligand and 475 nM unlabelled ligand. The assay buffer contained 20 mM Tris-HCl pH 7.5 (Invitrogen), 150 mM NaCl (Sigma Aldrich), 0.1 mM MgCl₂ (Sigma Aldrich), and 0.01% Tween-20 (Sigma Aldrich). Following addition of 50 μL of a 400 μg/mL suspension of streptavidin-coated YSi beads (Perkin Elmer), plates were incubated for a further 30 min with continuous agitation before reading the plates on a scintillation counter (Topcount NXT 384 (Packard).

Evaluation was carried out using assay data analysis software (such as the standard Novartis in-house Helios software application, Novartis Institutes for BioMedical Research, unpublished) using the methods described in Formenko et al., Robust Regression for high-throughput screening, Computer Methods and Programs in Biomedicine, 2006, 82, 31-37. Following normalization of activity values for the wells to % inhibition (% inhibition=[(high control−sample)/(high control−low control)]×100), IC₅₀ fitting was carried out from the duplicate determinations present on each plate according to Formenko et al., 2006.

Evaluation can also be carried out using commercially available software which is designed to derive IC₅₀ values using 4-parameter fits (e.g. GraphPad Prism, XL fit).

Titration of the non-labelled version of the probe, Compound A, in this assay resulted in an IC₅₀ of 0.5 □M. IC₅₀ values for examples 1 to 94 are tabulated in Table 2.

Note: Endpoint IC₅₀ values generated in this way could in principle be used to derive second order rate constants for the covalent binders in agreement with a method described by Miyahisa et al. 2015, Rapid Determination of the Specificity Constant of Irreversible Inhibitors (k_{inact}/K_{I}) by Means of an Endpoint Competition Assay, Angew. Chem, Int, Ed. Engl, 2015 Nov. 16; 54(47):14099-14102).

Following this, rate constants can be derived applying the equation (kinact/KI)inhibitor=(kinact/KI)probe×[probe]/IC50 using 0.5 μM as [probe]. “(k_{inact}/K_{I})probe”, the second order rate constant for modification of KRasG12C by this probe was internally determined to ˜5,000 M-1*s-1 for the non-labeled ligand [N-(3-fluoro-4-(2-methyl-3-(5-methyl-1H-indazol-4-yl)-1H-pyrrolo[2,3-b]pyridin-1-yl)phenyl)acrylamide (Compound A) using an MS-based assay (assessing % modification for a range of compound concentrations and time points).

TABLE 2
Tabulated in vitro KRASG12C activities

| | Covalent Competition |
|---|---|
| Example | Assay IC₅₀ (μM) |
| 1a | 0.086 |
| 1b | 10.7 |
| 2a | >50 |
| 2b | 3.2 |
| 3a | 0.340 |
| 3b | 50 |
| 4a | 0.242 |
| 4b | >50 |
| 5 | 2.2 |
| 6 | 1.0 |
| 7a | 0.210 |
| 7b | >50 |
| 8a | 0.151 |
| 8b | 37.9 |
| 9a | 0.346 |
| 9b | 50 |
| 10a | 50 |
| 10b | 0.105 |
| 11a | >50 |
| 11b | 0.120 |
| 12 | 1.4 |
| 13a | >50 |
| 13b | 2.1 |
| 14a | >50 |
| 14b | 0.364 |
| 15a | 18.0 |
| 15b | 0.305 |
| 16 | 1.0 |
| 17 | 0.829 |
| 18a | 6.1 |
| 18b | 0.031 |
| 19a | 0.894 |
| 19b | 34.7 |
| 20a | 0.301 |
| 20b | 38.3 |
| 21a | 11.1 |
| 21b | 0.017 |
| 22a | >50 |
| 22b | 0.113 |
| 23a | >50 |
| 23b | 0.634 |
| 24a | >50 |
| 24b | 0.167 |
| 25a | 0.553 |
| 25b | 20.8 |
| 26a | 11.5 |
| 26b | 0.110 |
| 27a | 2.7 |
| 27b | 0.024 |
| 28a | 0.076 |
| 28b | 1.3 |
| 29a | >5 |
| 29b | 0.032 |
| 30a | 0.514 |
| 30b | 2.7 |
| 31a | 3.7 |
| 31b | 0.023 |
| 32a | 2.2 |
| 32b | 0.030 |
| 33a | 0.043 |
| 33b | 4.1 |
| 34a | 3.8 |
| 34b | 0.013 |
| 35a | 0.254 |
| 35b | 1.5 |
| 36a | 24.4 |
| 36b | 2.4 |
| 37a | >50 |
| 37b | 2.1 |
| 38a | >5 |
| 38b | 0.089 |
| 39a | 42.1 |
| 39b | 0.075 |
| 40a | >50 |
| 40b | 0.633 |
| 41a | 41.9 |
| 41b | 0.149 |
| 42a | >50 |
| 42b | 0.186 |
| 43a | >50 |
| 43b | 0.758 |
| 44a | 4.6 |
| 44b | 0.020 |
| 45a | 0.365 |
| 45b | 1.5 |
| 46 | 0.138 |
| 47 | >5 |
| 48a | 0.296 |
| 48b | 0.006 |
| 49a | 1.787 |
| 49b | >5 |
| 50a | 0.273 |
| 50b | 0.005 |
| 51a | 0.010 |
| 51b | 0.668 |
| 52a | >5 |
| 52b | 0.030 |
| 53a | 4.9 |
| 53b | >50 |
| 53c | >50 |
| 53d | 9.031 |
| 54a | 17.8 |
| 54b | 0.015 |
| 55a | 24.7 |
| 55b | 0.034 |
| 56a | >50 |
| 56b | 0.334 |
| 57a | 34.7 |
| 57b | 0.216 |
| 58a | 2.8 |
| 58b | 0.030 |
| 59a | 4.5 |
| 59b | 0.022 |
| 60 | 4.9 |
| 61a | 0.881 |
| 61b | 0.006 |
| 62a | 0.446 |
| 62b | 0.010 |
| 63a | 0.010 |
| 63b | 3.1 |
| 64 | 0.519 |
| 65a | >5 |
| 65b | 0.212 |
| 66a | >50 |
| 66b | 2.2 |
| 67a | >50 |
| 67b | 0.038 |
| 68a | >15 |
| 68b | 0.239 |
| 69a | 4.4 |
| 69b | 0.015 |
| 70a | 0.287 |
| 70b | 1.0 |
| 71a | 1.2 |
| 71b | 0.025 |
| 72a | 3.1 |
| 72b | 0.007 |
| 73a | >5 |
| 73b | 0.015 |
| 74a | >5 |
| 74b | 0.026 |
| 75a | >5 |
| 75b | 0.228 |
| 76a | 0.351 |
| 76b | 0.010 |
| 77 | 1.2 |
| 78a | 1.3 |
| 78b | >5 |
| 79a | >5 |
| 79b | 0.018 |
| 80a | >5 |
| 80b | 0.007 |
| 81a | >5 |
| 81b | 0.585 |
| 82a | 50 |
| 82b | 0.831 |
| 83a | >5 |
| 83b | 0.026 |
| 84a | >5 |
| 84b | 0.014 |
| 85a | 5 |
| 85b | 0.008 |
| 86a | 2.3 |
| 86b | 0.008 |
| 87a | 0.013 |
| 87b | 1.5 |
| 88a | >5 |
| 88b | 0.467 |
| 89a | 3.4 |
| 89b | <0.005 |
| 90a | 0.023 |
| 90b | 0.558 |
| 91a | 2.9 |
| 91b | 0.013 |
| 92a | 2.1 |
| 92b | 0.007 |
| 93a | >5 |
| 93b | 0.012 |
| 94a | >50 |
| 94b | 0.064 |
| 95a | >5 |
| 95b | 0.373 |
| 96a | >5 |
| 96b | 0.011 |
| 97a | 0.3 |
| 97b | 1.7 |
| 98a | >5 |
| 98b | 0.019 |
| 99a | 2.9 |
| 99b | >5 |
| 100a | >5 |
| 100b | 0.086 |
| 101a | >50 |
| 101b | 0.103 |
| 102a | >5 |
| 102b | 0.023 |
| 103a | >5 |
| 103b | 0.315 |
| 104a | 4.441 |
| 104b | 0.027 |
| 105a | >5 |
| 105b | 0.019 |
| 106a | >5 |
| 106b | 0.014 |
| 107a | >5 |
| 107b | 0.010 |
| 108a | >5 |
| 108b | 0.576 |
| 109a | 0.334 |
| 109b | >5 |
| 110a | >5 |
| 110b | 0.010 |
| 111 | 1.7 |
| 112a | >5 |
| 112b | 0.171 |
| 113a | >5 |
| 113b | 0.105 |
| 114a | 0.007 |
| 114b | 0.337 |
| 115a | 0.007 |
| 115b | 0.246 |
| 116a | 0.205 |
| 116b | 1.9 |
| 117a | 0.009 |
| 117b | 1.6 |
| 118a | 0.015 |
| 118b | 0.818 |
| 119a | 2.8 |
| 119b | 0.036 |
| 120a | >5 |
| 120b | 0.14 |
| 121a | >5 |
| 121b | 0.008 |
| 122a | >5 |
| 122b | 0.016 |
| 123a | >5 |
| 123b | 0.021 |
| 124a | >5 |
| 124b | 0.020 |
| 125a | 2.2 |
| 125b | 0.035 |
| 126a | >50 |
| 126b | 0.170 |
| 127a | 25.2 |
| 127b | 0.020 |
| 128a | >50 |
| 128b | 0.054 |
| 129a | 0.032 |
| 129b | 1.2 |
| 130a | >5 |
| 130b | 0.032 |
| 131a | >5 |
| 131b | 0.076 |
| 132a | 0.697 |
| 132b | 4.5 |
| 132c | 0.024 |
| 132d | 0.029 |
| 133a | 1.6 |
| 133b | 0.005 |
| 134a | >5 |
| 134b | 0.046 |
| 135a | >5 |
| 135b | 0.014 |
| 136a | 0.846 |
| 136b | >5 |
| 137a | >5 |
| 137b | 0.975 |
| 138a | >5 |
| 138b | 2.782 |
| 139 | 0.450 |
| 140a | 0.971 |
| 140b | 0.018 |
| 141a | >5 |
| 141b | 0.104 |
| 142a | 0.204 |
| 142b | >5 |
| 143a | >5 |
| 143b | 0.039 |
| 144a | >5 |
| 144b | 0.010 |
| 145a | 4.3 |
| 145b | 0.012 |
| 146a | >5 |
| 146b | 0.031 |
| 147a | 0.008 |
| 147b | 0.923 |
| 148a | >5 |
| 148b | 0.295 |
| 149a | 0.766 |
| 149b | 0.009 |
| 150a | 0.252 |
| 150b | <0.005 |
| 151a | 0.046 |
| 151b | >5 |
| 152a | >5 |
| 152b | 0.045 |
| 153a | >5 |
| 153b | 0.019 |
| 154a | >5 |
| 154b | 0.022 |
| 155a | 4.0 |
| 155b | 0.005 |
| 156a | >5 |
| 156b | 0.008 |
| 157a | >5 |
| 157b | >5 |
| 157c | 0.006 |
| 157d | 0.007 |
| 158a | 0.006 |
| 158b | 0.172 |
| 159a | 0.715 |
| 159b | 0.009 |
| 160a | >5 |
| 160b | 0.027 |
| 161a | 0.912 |
| 161b | 0.008 |
| 162a | >5 |
| 162b | 0.027 |
| 163a | >5 |
| 163b | 0.114 |
| 164a | >5 |
| 164b | 0.056 |
| 165a | >5 |
| 165b | 0.026 |
| 166a | >5 |
| 166b | 0.051 |
| 167a | >5 |
| 167b | 0.031 |
| 168 | 0.302 |
| 169a | >5 |
| 169b | 0.012 |
| 170a | >50 |
| 170b | 0.060 |
| 171a | >5 |
| 171b | 0.008 |
| 172a | 3.9 |
| 172b | 0.015 |
| 173a | 2.9 |
| 173b | 0.005 |
| 174a | >5 |
| 174b | 0.121 |
| 175a | 0.939 |
| 175b | 0.009 |
| 176 | <0.005 |
| 177a | 0.008 |
| 177b | 1.7 |
| 178a | 3.9 |
| 178b | 0.048 |
| 179a | >5 |
| 179b | 0.038 |
| 180a | >5 |
| 180b | 0.017 |
| 181a | 0.929 |
| 181b | 0.008 |
| 182a | 1.4 |
| 182b | 0.008 |
| 183a | >5 |
| 183b | 0.027 |
| 184a | >5 |
| 184b | 0.061 |
| 185a | 4.1 |
| 185b | 0.015 |
| 186a | 1.5 |
| 186b | 0.007 |
| 187a | 4.2 |
| 187b | 0.005 |
| 188a | 0.387 |
| 188b | 0.006 |
| 189a | >5 |
| 189b | 0.015 |
| 190a | 4.5 |
| 190b | 0.111 |
| 191a | >5 |
| 191b | 0.014 |
| 192a | >5 |
| 192b | 0.008 |
| 193a | 20.8 |
| 193b | 0.028 |
| 194a | 0.019 |
| 194b | 1.3 |
| 195a | 10.4 |
| 195b | >50 |
| 196a | >50 |
| 196b | 9.0 |
| 197a | >50 |
| 197b | 49.7 |
| 198a | >50 |
| 198b | >50 |
| 199a | >5 |
| 199b | 0.037 |
| 200a | >5 |
| 200b | 0.023 |
| 201a | 4.3 |
| 201b | 0.017 |
| 202a | 2.0 |
| 202b | 0.033 |
| 203a | 0.353 |
| 203b | 2.8 |
| 204a | 1.4 |
| 204b | 0.041 |
| 205a | 0.449 |
| 205b | 2.9 |
| 206a | 0.954 |
| 206b | 0.023 |
| 207a | >5 |
| 207b | 0.02 |
| 208a | 0.064 |
| 208b | 3.8 |
| 209a | 1.1 |
| 209b | 0.012 |
| 210a | 2.2 |
| 210b | 0.010 |
| 211a | 1.6 |
| 211b | 0.008 |
| 212a | 0.358 |
| 212b | 0.012 |
| 213a | 3.5 |
| 213b | 0.012 |
| 214a | 0290 |
| 214b | 0.679 |
| 215a | 2.2 |
| 215b | 0.016 |
| 216a | 0.443 |
| 216b | 1.3 |
| 217a | 0.928 |
| 217b | 0.008 |
| 218a | 0.081 |
| 218b | >5 |
| 219a | 1.6 |
| 219b | 0.042 |
| 220a | >5 |
| 220b | 0.036 |
| 221a | 0.005 |
| 221b | 0.405 |
| 222a | 1.1 |
| 222b | 0.007 |
| 223 | 0.600 |
| 224a | >5 |
| 224b | 0.012 |
| 225a | 0.341 |
| 225b | >5 |
| 226a | 0.119 |
| 226b | 1.1 |
| 227a | 4.9 |
| 227b | 0.008 |
| 228a | >5 |
| 228b | 0.281 |
| 229a | >5 |
| 229b | 0.011 |
| 230a | >5 |
| 230b | 0.017 |
| 231a | >5 |
| 231b | 0.011 |
| 232a | >5 |
| 232b | 0.137 |
| 233a | 4.0 |
| 233b | 0.020 |
| 234a | 1.0 |
| 234b | 0.009 |
| 235a | 0.932 |
| 235b | 0.010 |
| 236a | 0.168 |
| 236b | 2.5 |
| 237a | >5 |
| 237b | 0.031 |
| 238a | 0.036 |
| 238b | 0.503 |
| 239a | 1.6 |
| 239b | 0.009 |
| 240a | 0.390 |
| 240b | 0.008 |
| 241a | 0.008 |
| 241b | 0.536 |
| 242a | 0.013 |
| 242b | 1.4 |
| 243a | 3.8 |
| 243b | 0.006 |
| 244a | 1.4 |
| 244b | 0.006 |
| 245a | 1.8 |
| 245b | 0.014 |
| 246a | >5 |
| 246b | 0.015 |
| 247a | 1.4 |
| 247b | 0.009 |
| 248a | >5 |
| 248b | 0.009 |
| 249a | >5 |
| 249b | 0.013 |
| 250a | 0.405 |
| 250b | 0.008 |
| 251a | >5 |
| 251b | 0.009 |
| 252a | >5 |
| 252b | 0.006 |
| 253a | 1.5 |
| 253b | 0.008 |
| 254a | 2.6 |
| 254b | 0.008 |
| 255a | 3.3 |
| 255b | 0.084 |
| 256a | 1.3 |
| 256b | 0.010 |
| 257a | nd |
| 257b | 0.012 |
| 258a | nd |
| 258b | 0.022 |
| 259a | 0.779 |
| 259b | 0.010 |
| 260a | >5 |
| 260b | 0.009 |
| 261a | >5 |
| 261b | 0.015 |
| 262a | >5 |
| 262b | 0.007 |
| 263a | >5 |
| 263b | 0.008 |
| 264a | 0.351 |
| 264b | 0.033 |
| 265a | 2.9 |
| 265b | 0.009 |
| 266a | 3.1 |
| 266b | 0.007 |
| 267a | 0.720 |
| 267b | 0.007 |
| 268a | 0.789 |
| 268b | 0.007 |
| 269a | 4.3 |
| 269b | 0.006 |
| 270a | 2.4 |
| 270b | 0.005 |
| 271a | >5 |
| 271b | 0.014 |
| 272a | >5 |
| 272b | 0.008 |
| 273a | 1.1 |
| 273b | 0.010 |
| 274a | 0.795 |
| 274b | 0.008 |
| 275a | >5 |
| 275b | 0.007 |
| 276a | 3.4 |
| 276b | <0.005 |
| 277a | 4.5 |
| 277b | <0.005 |
| 278a | >5 |
| 278b | 0.008 |
| 279a | 3.8 |
| 279b | 0.006 |
| 280a | >5 |
| 280b | 0.007 |
| 281a | 1.6675859 |
| 281b | <0.005 |
| 282a | 1.5 |
| 282b | 0.007 |
| 283a | 3.0 |
| 283b | 0.008 |
| 284a | 1.6 |
| 284b | 0.008 |
| 285a | 4.6 |
| 285b | 0.007 |
| 286a | 1.1 |
| 286b | 0.007 |
| 287a | 0.307 |
| 287b | 0.009 |
| 288a | 4.9 |
| 288b | 0.010 |
| 289a | 3.3 |
| 289b | 0.010 |
| 290a | 3.4 |
| 290b | <0.005 |
| 291a | >5 |
| 291b | 0.008 |
| 292a | 1.9 |
| 292b | 0.027 |
| 293a | 4.0 |
| 293b | 0.010 |
| 294a | 4.7 |
| 294b | 0.010 |
| 295a | 2.0 |
| 295b | 0.006 |
| 296a | 0.392 |
| 296b | 0.006 |
| 297a | >5 |
| 297b | 0.009 |
| 298a | 1.0 |
| 298b | 0.027 |
| 299a | 0.793 |
| 299b | 0.023 |
| 300a | >5 |
| 300b | 0.013 |
| 301a | 1.9 |
| 301b | <0.005 |
| 302a | >5 |
| 302b | 0.010 |
| 303a | 5 |
| 303b | 0.005 |
| 304a | >5 |
| 304b | 0.010 |
| 305a | 2.5 |
| 305b | 0.019 |
| 306a | >5 |
| 306b | 0.005 |
| 307a | >5 |
| 307b | 0.008 |
| 308a | 0.747 |
| 308b | 0.010 |
| 309a | 1.1 |
| 309b | 0.015 |
| 310a | 2.9 |
| 310b | 0.007 |
| 311a | 1.3 |
| 311b | 0.018 |
| 312a | 0.831 |
| 312b | 0.009 |
| 313a | 0.410 |
| 313b | 0.006 |
| 314a | 2.1 |
| 314b | 0.052 |
| 315a | 2.0 |
| 315b | 0.007 |
| 316a | 1.2 |
| 316b | 0.009 |
| 317a | 0.888 |
| 317b | 0.007 |
| 318a | 4.0 |
| 318b | 0.007 |
| 319a | 2.7 |
| 319b | 0.009 |
| 320a | 3.3 |
| 320b | <0.005 |

1. A compound of formula (I), or a pharmaceutically acceptable salt thereof: 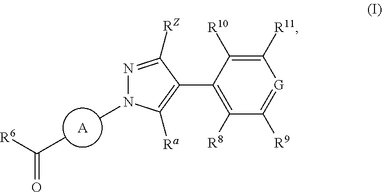
wherein:
Ring A is a 6 to 10 membered spirocyclic-heterocyclylene comprising 1 to 3 heteroatoms independently selected from N, O and S, wherein said 6 to 10 membered spirocyclic-heterocyclylene is substituted with 0 to 3 substituents R¹⁶;
G is N or CR¹²;
R^{Z} is 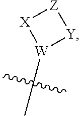
wherein
W is N;
i) X is **—CR²₂—(CR³₂)ₙ—* or **—CR²═CR³—*, Y is **—CR⁴₂—(CR⁵₂)ₘ—*, and Z is selected from the group consisting of S(O)₂, S, S(O), O, P(O)—C₁-C₃alkyl, NR^{1N} and C(R^{1C})₂, where the * of X indicates the point of attachment to Z and the ** of X indicates the point of attachment to W, and where the * of Y indicates point of attachment to Z and the ** of Y indicates point of attachment to W, n is 0, 1 or 2 and m is 0, 1 or 2; or
ii) X is **—CR²₂—CR³═*, Y is **—CR⁴₂—(CR⁵₂)ₘ—*, and Z is selected from the group consisting of N and CR^{1C}, where the * of X indicates the point of attachment to Z and the ** of X indicates the point of attachment to W, and where the * of Y indicates point of attachment to Z and the ** of Y indicates point of attachment to W, and m is 0, 1 or 2;
R^{1N} is selected from the group consisting of H and -L^{N}-R^{2N};
an R^{1N} group and one or two R³ groups, in combination with the atoms to which they are mutually attached, form a saturated or unsaturated 5 or 6 membered ring containing one to three heteroatoms selected from the group consisting of N, O, S and P, wherein said saturated or unsaturated 5 or 6 membered ring containing one to three heteroatoms is substituted with 0 to 3 substituents R^{x}; or
an R^{1N} group and one or two R⁵ groups, in combination with the atoms to which they are mutually attached, form a saturated or unsaturated 5 or 6 membered ring containing one to three heteroatoms selected from the group consisting of N, O, S and P, wherein said saturated or unsaturated 5 or 6 membered ring containing one to three heteroatoms is substituted with 0 to 3 substituents R^{x};
R^{1C}, where present, is at each occurrence independently selected from the group consisting of H and -L^{C}-R^{2C};
one or two R^{1C} group(s), and one or two R³ groups, in combination with the carbon atoms to which they are mutually attached, form a saturated or unsaturated 5 or 6 membered ring containing zero to three heteroatoms selected from the group consisting of N, O, S and P, wherein said saturated or unsaturated 5 or 6 membered ring containing zero to three heteroatoms is substituted with 0 to 3 substituents R^{x};
one or two R^{1C} groups, and one or two R⁵ groups, in combination with the carbon atoms to which they are mutually attached, form a saturated or unsaturated 5 or 6 membered ring containing zero to three heteroatoms selected from the group consisting of N, O, S and P, wherein said saturated or unsaturated 5 or 6 membered ring containing zero to three heteroatoms is substituted with 0 to 3 substituents R^{x};
two R^{1C} groups together form oxo; or
two R^{1C} groups together with the carbon atom to which they are mutually attached form a C₄-C₈cycloalkyl or a 4 to 6 membered heterocyclyl comprising 1 to 3 heteroatoms independently selected from N, O, S and P, said C₄-C₆cycloalkyl or 4 to 6 membered heterocyclyl being substituted with 0 to 2 substituents R^{x};
L^{N} is selected from the group consisting of a bond, C═O, C₁-C₆alkylene, SO₂, C(═O)—O*, C(═O)—C₁-C₆alkylene*, C₁-C₆alkylene-C(═O)* and C(═O)—O—C₁-C₆alkylene*, wherein * indicates the point of attachment to R^{2N},
R^{2N} is selected from the group consisting of:
i) C₁-C₆alkyl substituted with 0 to 3 substituents R^{x},
ii) 3-10 membered heterocyclyl comprising 1 to 3 heteroatoms independently selected from N, O, S and P substituted with 0 to 3 substituents R^{x},
iii) 6 to 10 membered spirocyclic-heterocyclyl comprising 1 to 3 heteroatoms independently selected from N, O, S and P substituted with 0 to 3 substituents R^{x},
iv) hydroxyl,
v) C₁-C₆haloalkyl,
vi) aryl substituted with 0 to 2 substituents R^{x},
vii) O—C₁-C₆haloalkyl,
viii) O—C₁-C₆alkyl,
ix) 5-6 membered heteroaryl comprising 1 to 3 heteroatoms independently selected from N, O and S substituted with 0 to 2 substituents R^{x},
x) C₃-C₅cycloalkyl substituted with 0 to 2 substituents R^{x},
xi) N(C₁-C₆alkyl)₂ or NH(C₁-C₆alkyl),
xii) CH(C₁-C₆alkylene-O—C₁-C₆alkyl)₂, and
xiii) CN;
L^{C} is selected from the group consisting of a bond, C═O, C₁-C₆alkylene or O—C₁-C₆alkylene*, wherein * indicates the point of attachment to R^{2C},
wherein R^{2C} is at each occurrence independently selected from the group consisting of
i) C₁-C₆ alkyl substituted by 0 to 3 substituents R^{x},
ii) hydroxyl,
iii) 6 to 10 membered spirocyclic-heterocyclyl comprising 1 to 3 heteroatoms independently selected from N, O, S and P substituted by 0 to 3 substituents R^{x},
iv) 5-6 membered heteroaryl comprising 1 to 3 heteroatoms independently selected from N, O and S substituted by 0 to 2 substituents R^{x},
v) 3-10 membered heterocyclyl comprising 1 to 3 heteroatoms independently selected from N, O, S and P, substituted by 0 to 3 substituents R^{x} or wherein the 3-10 membered heterocyclyl is perdeuterated,
vi) NR^{1A}R^{1B}, and
vii) 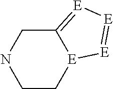
wherein E at each occasion is independently selected from CH and N substituted by 0 to 2 substituents R^{x},
R^{1A} and R^{1B} are each independently selected from the group consisting of H, C₁-C₆alkyl, C₁-C₆hydroxyalkyl, C₁-C₆haloalkyl, C₁-C₆alkylene-O—C₁-C₆alkyl, C₃-C₅cycloalkyl substituted by 0 to 2 substituents R^{x}, 3-10 membered heterocyclyl comprising 1 to 3 heteroatoms independently selected from N, O, S and P substituted by 0 to 2 substituents R^{x}, C₁-C₆alkylene-C₃-C₈cycloalkyl substituted by 0 to 2 substituents R^{x}, C₁-C₆alkylene-3-10 membered heterocyclyl comprising 1 to 3 heteroatoms independently selected from N, O, S and P substituted by 0 to 2 substituents R^{x}, SO₂-3-10 membered heterocyclyl comprising 1 to 3 heteroatoms independently selected from N, O, S and P substituted by 0 to 2 substituents R^{x}, 6 to 10 membered spirocyclic-heterocyclyl comprising 1 to 3 heteroatoms independently selected from N, O, S and P substituted by 0 to 2 substituents R^{x}, aryl substituted by 0 to 2 substituents R^{x}, 5-6 membered heteroaryl comprising 1 or 2 heteroatoms independently selected from N, O and S substituted by 0 to 2 substituents R^{x}, C₁-C₆alkylene-aryl substituted by 0 to 2 substituents R^{x}, C₁-C₆alkylene-5-6 membered heteroaryl comprising 1 or 2 heteroatoms independently selected from N, O and S substituted by 0 to 2 substituents R^{x}, C(═O)—C₁-C₆alkyl, C(═O)—C₁-C₆alkylene-O—C₁-C₆alkyl, and C₁-C₆alkylene-C(═O)-3-10 membered heterocyclyl comprising 1 to 3 heteroatoms independently selected from N, O, S and P substituted by 0 to 2 substituents R^{x};
R², R³, R⁴ and R⁵ are each independently selected from the group consisting of H, C₁-C₆alkyl, C₃-C₈cycloalkyl, halo, C₁-C₆alkylene-O—C₁-C₆alkyl, C(═O)—C₁-C₅alkyl, C₁-C₆haloalkyl, hydroxyl, C₁-C₆hydroxyalkyl, NR^{1P}R^{1Q}, C₁-C₆alkylene-NR^{1P}R^{1Q}, cyano, C₁-C₆cyanoalkyl, C₁-C₆alkylene-O—C₁-C₆haloalkyl, C(═O)—NHC₁-C₅alkyl, C(═O)—N(C₁-C₅alkyl)₂, and C(═O)—O—C₁-C₅alkyl,
wherein R^{1P} and R^{1Q} are each independently selected from the group consisting of H, C(═O)—C₁-C₆alkyl, C₁-C₆alkyl, C₁-C₆alkylene-O—C₁-C₆alkyl, C₁-C₈hydroxyalkyl or wherein R^{1P} and R^{1Q} together with the nitrogen atom to which they are mutually attached form a 4 to 6 membered heterocyclyl comprising 1 or 2 heteroatoms independently selected from N, O, S;
i) an R² group and an R⁴ group in combination form a bridging group;
ii) an R² group and an R⁵ group in combination form a bridging group;
iii) an R³ group and an R⁴ group in combination form a bridging group; or
iv) an R³ group and an R⁵ group in combination form a bridging group;
wherein the bridging group forms a C₄-C₈cycloalkyl, or a 4 to 6 membered heterocyclyl comprising 1 to 3 heteroatoms independently selected from the group consisting of N, O, S and P, wherein the C₄-C₆cycloalkyl or 4 to 6 membered heterocyclyl are each substituted with 0 to 3 substituents R^{x}; or
i) an R² group and an R³ group in combination with the carbon atoms to which they are mutually attached form a ring; and/or
ii) an R⁴ group and an R⁵ group in combination with the carbon atoms to which they are mutually attached form a ring;
wherein the ring is a C₄-C₆cycloalkyl, or a 4 to 6 membered heterocyclyl comprising 1 to 3 heteroatom independently selected from the group consisting of N, O, S and P, wherein the C₄-C₆cycloalkyl or 4 to 6 membered heterocyclyl are each substituted with 0 to 3 substituents R^{x}; or
i) two R² groups in combination form an oxo or in combination with the carbon atom to which they are mutually attached form a ring;
ii) two R³ groups in combination form an oxo or in combination with the carbon atom to which they are mutually attached form a ring;
iii) two R⁴ groups in combination form an oxo or in combination with the carbon atom to which they are mutually attached form a ring; or
iv) two R⁵ groups in combination form an oxo or in combination with the carbon atom to which they are mutually attached form a ring;
wherein the ring is a C₃-C₆cycloalklyl or a 3 to 6 membered heterocyclyl comprising 1 or 2 heteroatoms independently selected from the group consisting of N, O, S and P, wherein the C₃-C₆cycloalkyl or 3 to 6 membered heterocyclyl is substituted with 0 to 3 substituents R^{x};
each R^{x} is independently selected from a) C₁-C₃alkyl, b) halo, c) C(═O)—C₁-C₃alkyl, d) C(═O)—C₁-C₃hydroxyalkyl, e) cyano, f) hydroxyl, g) amino, h) oxo, i) O—C₁-C₃alkyl, j) C₁-C₃hydroxyalkyl, k) C₁-C₃haloalkyl, l) O—C₁-C₃haloalkyl, m) COOH, n) SO₂—C₁-C₃alkyl, o) C₁-C₃alkylene-O—C₁-C₃alkyl, p) C₃-C₈cycloalkyl substituted by 0 to 2 substituents selected from the group consisting of CH₃, OH, OMe, F and CN, q) 3 to 6 membered heterocyclyl comprising 1 to 3 heteroatoms independently selected from the group consisting of N, O and S substituted by 0 to 2 substituents selected from the group consisting of CH₃, OH, OMe, F and CN, r) NR^{Xa}R^{Xb}, s) C(═O)—NR^{Xa}R^{Xb}, and t) deuterium;
wherein R^{Xa} and R^{Xb} are independently selected from the group consisting of H, C(═O)—C₁-C₆alkyl, SO₂—C₁-C₃alkyl, C₂-C₄haloalkyl, C₂-C₄alkylene-O—C₁-C₃alkyl, C₁-C₃alkyl and 3 to 6 membered heterocyclyl comprising 1 to 3 heteroatoms independently selected from the group consisting of N, O and S;
R⁶ is CR^{7a}═CR^{7b}₂, C≡CR^{7b}, or CR^{7c}₃;
R^{7a}, where present, is H or fluoro;
each R^{7b} is independently selected from the group consisting of H, halo and C(R^{7d})₃ wherein each R^{7d} is independently selected from the group consisting of H, halo, O—C₁-C₆alkyl, C₁-C₆alkyl, hydroxyl, and NR^{7e}R^{7f}, wherein R^{7e} and R^{7f} are each H or C₁-C₆alkyl, or wherein R^{7e} and R^{7f} together with the nitrogen atom to which they are mutually attached form a 3 to 8 membered heterocyclyl comprising 1 to 3 heteroatoms each independently selected from the group consisting of N, O, S and P, with at least one heteroatom being nitrogen, with the proviso that if one R^{7d} substituent is selected from the group consisting of O-C₁-C₆ alkyl, hydroxyl and NR^{7e}R^{7f}, the other two R^{7d} substituents are both H;
one R^{7c} is selected from the group consisting of H, halo and C₁-C₆alkyl and the other two R^{7c} groups in combination with the carbon atom to which they are mutually attached form a 3 membered heterocyclyl comprising 1 heteroatom selected from the group consisting of N and O;
R⁸ is H, halo, O—C₁-C₃alkyl, C₃-C₄cycloalkyl, 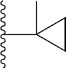 [SMILES: *C1(C)CC1]
or C(R^{8a})₃, wherein each R^{8a} is independently selected from the group consisting of H, C₁-C₃alkyl, and halo,
R⁹ is H, halo, NH₂, hydroxyl, C₃-C₄cycloalkyl or C(R^{9a})₃, wherein each R^{9a} is independently selected from the group consisting of H, C₁-C₃alkyl, and halo, or R⁸ and R⁹ together with the aryl ring to which they are mutually attached form 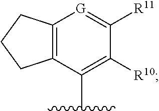
R¹⁰ is selected from the group consisting of H, halo, NH₂, C₁-C₃alkyl, and hydroxyl;
R¹¹ is selected from the group consisting of H, halo, NH₂, hydroxyl and C₁-C₃alkyl, or
R¹⁰ and R¹¹ are joined together to form, in combination with the 6 membered aryl or heteroaryl to which they are mutually attached, a 9 or 10 membered fused bicyclic aryl or heteroaryl group containing 1 to 3 heteroatoms independently selected from the group consisting of N, O, and S, wherein said fused bicyclic heteroaryl group is substituted with 0 to 3 substituents independently selected from the group consisting of C₁-C₆alkyl, NH₂, R¹⁴, R¹⁵, R¹⁷, R¹⁸, R¹⁹ and R²⁰;
R¹² is H, halo or methyl;
R^{a} is H, CN or C(R¹³)₃,
each R¹³ is independently selected from the group consisting of H, deuterium, halo, C₁-C₃alkyl and hydroxyl, provided that no more than one R¹³ is hydroxyl,
or two R¹³ substituents in combination with the carbon atom to which they are mutually attached form a C₃-C₅cycloalkyl or a 3 to 5 membered heterocyclyl comprising 1 to 3 heteroatoms each independently selected from the group consisting of N, O, S and P and the third R₁₃ substituent is H, halo, C₁-C₃alkyl or hydroxyl,
R¹⁴ is selected from the group consisting of H and C₁-C₃alkyl;
R¹⁵, R¹⁷, R¹⁸, R¹⁹ and R²⁰ are each independently selected from the group consisting of H, halo, C₁-C₃alkyl and NH₂; and
each R¹⁶ group is independently selected from the group consisting of C₁-C₃alkyl, cyano, halo, hydroxyl, O—C₁-C₃alkyl, C₁-C₃haloalkyl, C₁-C₃hydroxyalkyl, and C₁-C₃cyanoalkyl.

2-4. (canceled)

5. The compound according to claim 1, wherein Ring A is 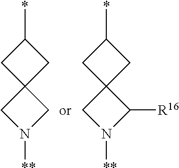
wherein * denotes the point of attachment to the pyrazole ring and ** denotes the point of attachment to —C(═O)R⁶, and wherein R¹⁶ is C₁-C₃alkyl, or a pharmaceutically acceptable salt thereof.

6-16. (canceled)

17. The compound according claim 1, wherein 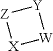
is selected from the group consisting of: 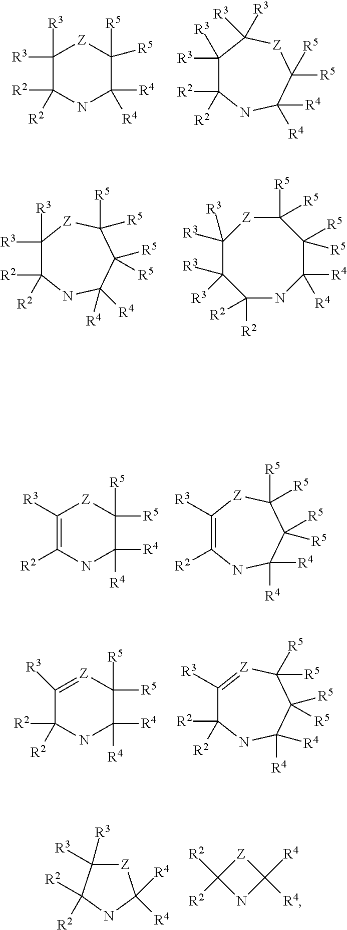
or a pharmaceutically acceptable salt thereof.

18. The compound according to claim 17, wherein 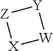
is selected from the group consisting of: 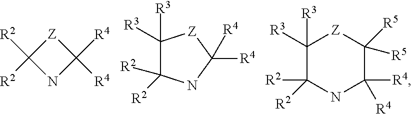
or a pharmaceutically acceptable salt thereof.

19. The compound according to claim 1, wherein R⁶ is CR^{7a}═C(R^{7b})₂, or a pharmaceutically acceptable salt thereof.

20-23. (canceled)

24. The compound according to claim 1, wherein R¹⁰ and R¹¹ are joined together, in combination with the 6 membered aryl or heteroaryl to which they are mutually attached, to form a fused bicyclic aryl or heteroaryl group selected from the group consisting of: 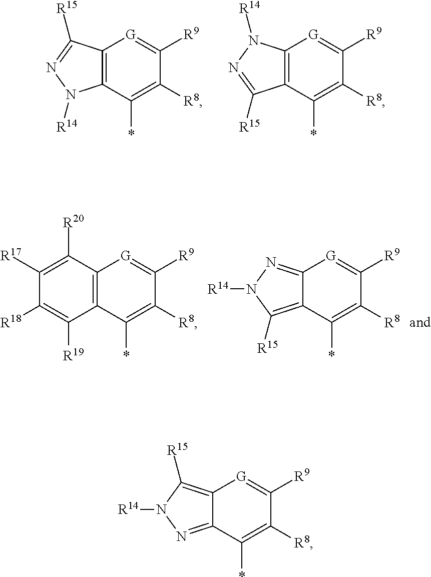
and wherein * denotes where the fused bicyclic heteroaryl group is attached to the remainder of the molecule, or a pharmaceutically acceptable salt thereof.

25. The compound according to claim 24 wherein R¹⁰ and R¹¹ are joined together with the 6 membered aryl or heteroaryl to which they are attached to form a fused bicyclic aryl or heteroaryl group selected from the group consisting of 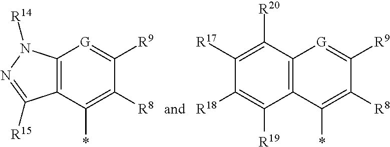
or a pharmaceutically acceptable salt thereof.

26. The compound according to claim 25 wherein R¹⁰ and R¹¹ are joined together with the 6 membered aryl or heteroaryl to which they are attached to form the fused bicyclic heteroaryl group 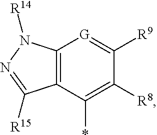
or a pharmaceutically acceptable salt thereof.

27-47. (canceled)

48. The compound according to claim 1 wherein the compound is a compound of formula (II) or (IIa) 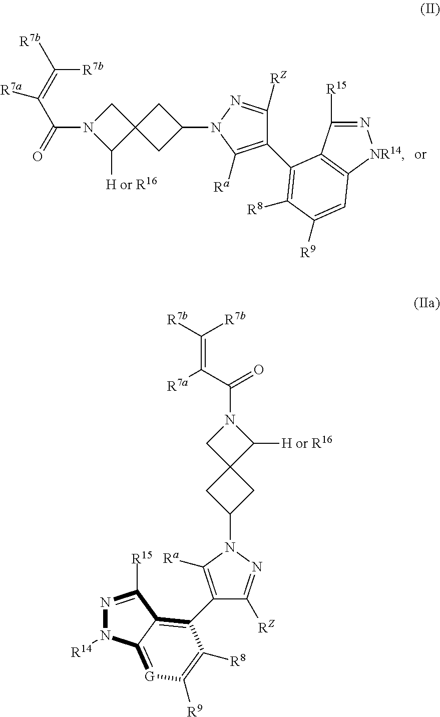
wherein R^{a} is C(R¹³)₃, and
R^{Z} is selected from the group consisting of: 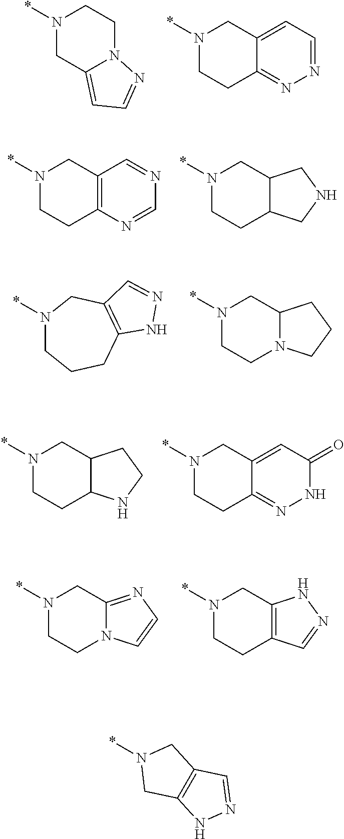
wherein * indicates the point of attachment to the remainder of the molecule,
and wherein any of the above R^{Z} groups are substituted with 0 to 3 substituents independently selected from the group consisting of C₁-C₃alkyl, C₁-C₃haloalkyl, oxo (═O), C(═O)—C₁-C₃alkyl, cyano, and halo,
or R^{Z} is selected from 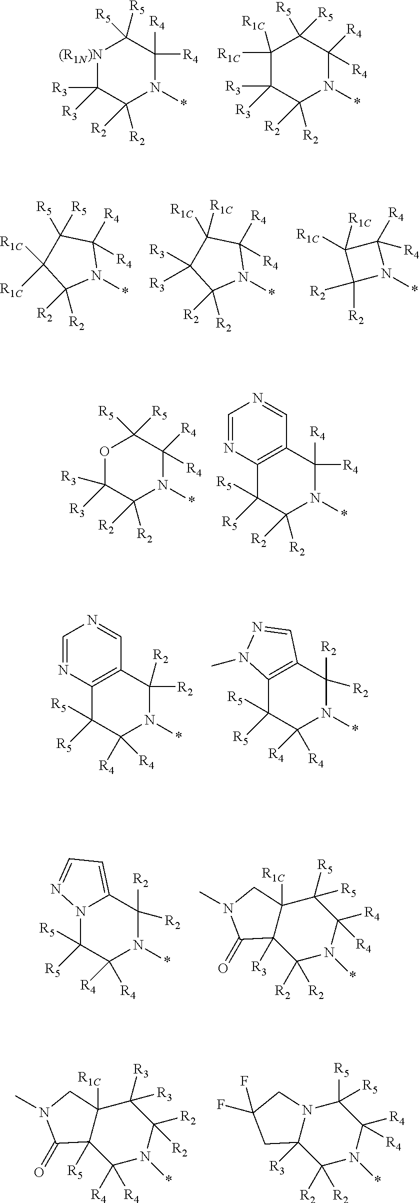 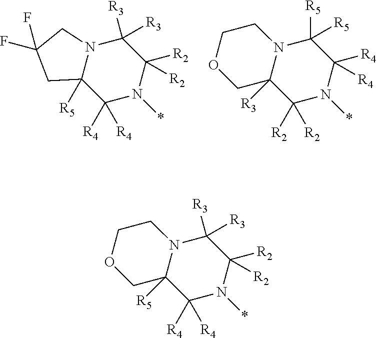
or a pharmaceutically acceptable salt thereof.

49. The compound according to claim 1 wherein the compound according to formula (I) is a compound according to formula (III) or (IIIa) 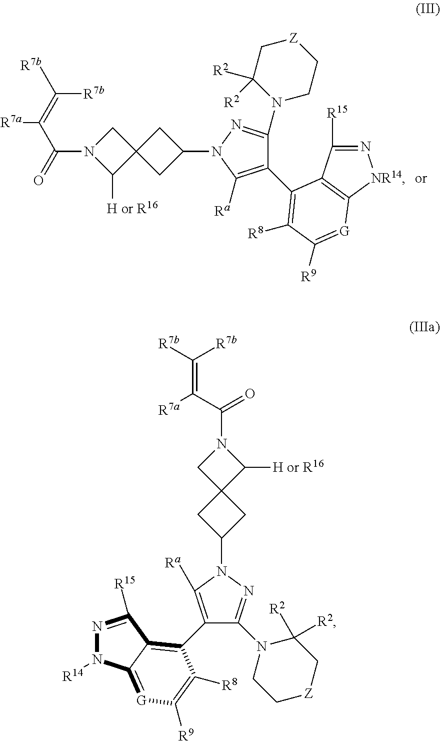
Z is selected from the group consisting of S, S(O), S(O)₂, NR^{1N} and C(R^{1C})₂, and
each R² is independently selected from the group consisting of C₁-C₃ alkyl, C₁-C₃fluoroakyl or C₁-C₃alkylene-O—C₁-C₃alkyl or wherein, where present, two R² groups in combination with the carbon atom to which they are mutually attached form a C₄-C₅cycloalkyl or 4 to 6 membered heterocyclyl comprising 1 heteroatom which is N or O, wherein the C₄-C₅cycloalkyl or 4 to 6 membered heterocyclyl is unsubstituted or substituted with C(═O)—CH₃,
or a pharmaceutically acceptable salt thereof.

50-51. (canceled)

52. The compound according to claim 1, wherein R^{1N} is selected from the group consisting of C(═O)—CH₃, 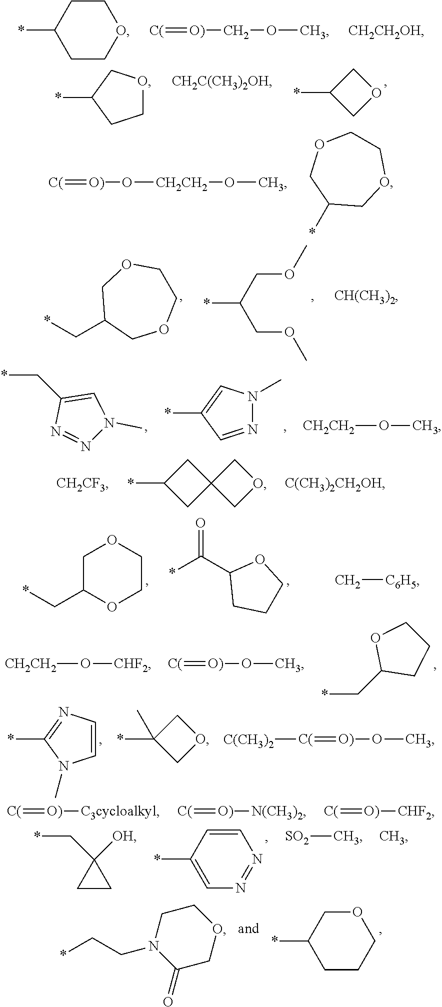
wherein * indicates the point of attachment to the remainder of the molecule, or a pharmaceutically acceptable salt thereof.

53-56. (canceled)

57. The compound according to claim 1 selected from the group consisting of: 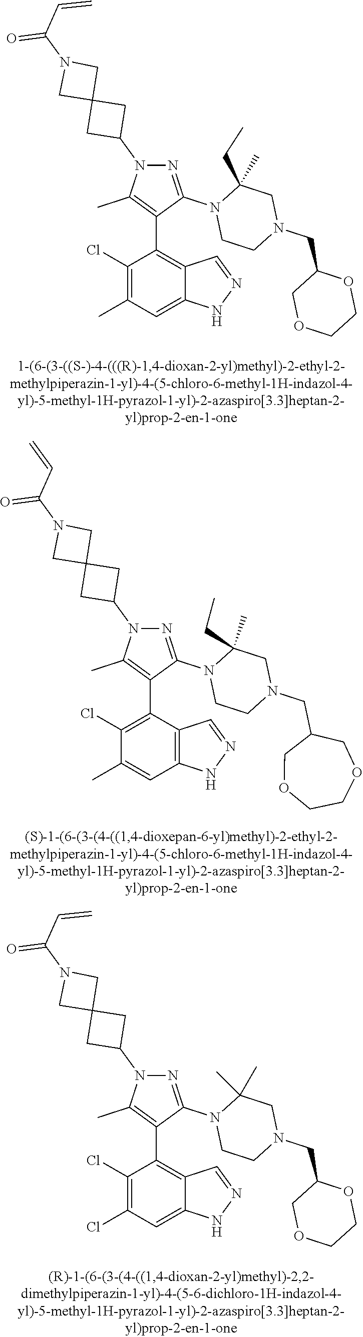 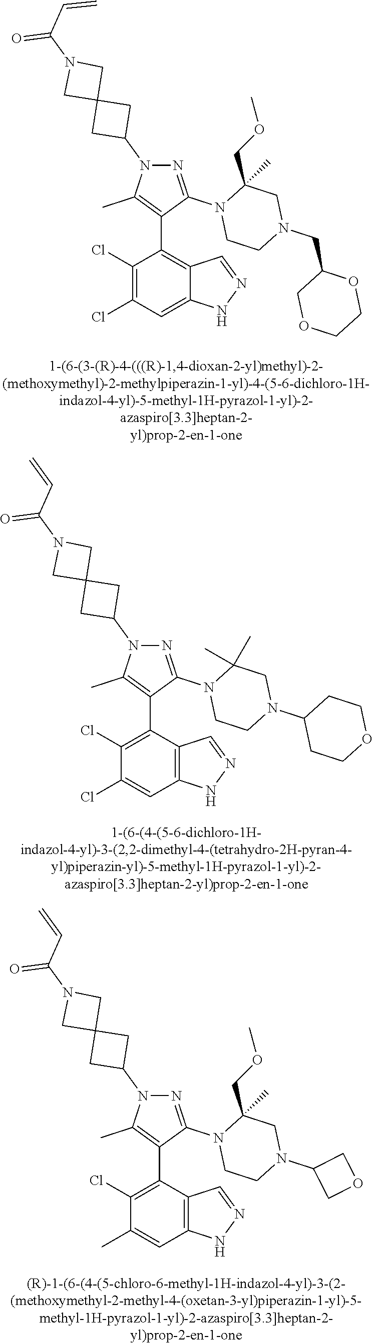 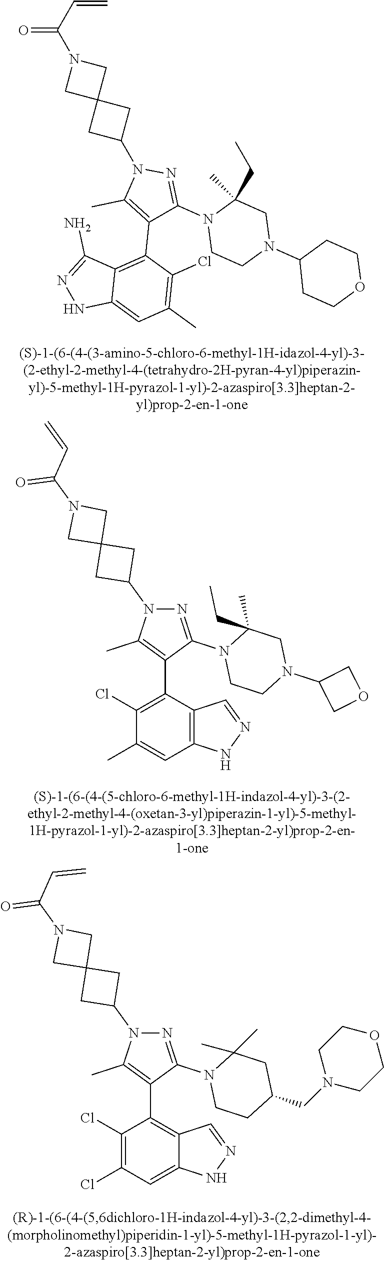 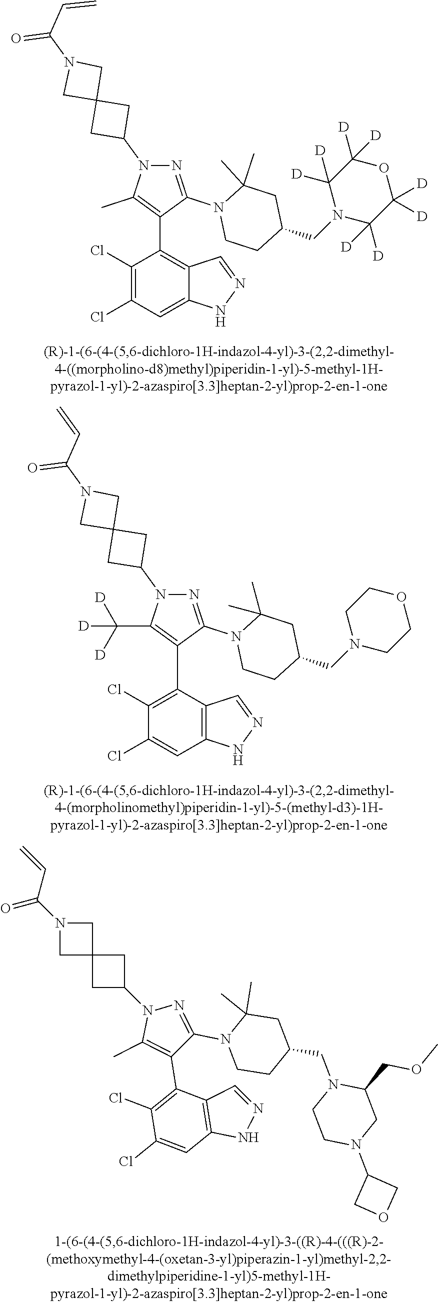 [SMILES: [2H]C1([2H])OC([2H])([2H])C([2H])([2H])N(C[C@@H]2CCN(c3nn(C4CC5(C4)CN(C(=O)C=C)C5)c(C)c3-c3c(Cl)c(Cl)cc4[nH]ncc34)C(C)(C)C2)C1([2H])[2H] ; [2H]C([2H])([2H])c1c(-c2c(Cl)c(Cl)cc3[nH]ncc23)c(N2CC[C@@H](CN3CCOCC3)CC2(C)C)nn1C1CC2(C1)CN(C(=O)C=C)C2 ; C=CC(=O)N1CC2(CC(n3nc(N4CC[C@@H](CN5CCN(C6COC6)C[C@@H]5COC)CC4(C)C)c(-c4c(Cl)c(Cl)cc5[nH]ncc45)c3C)C2)C1] 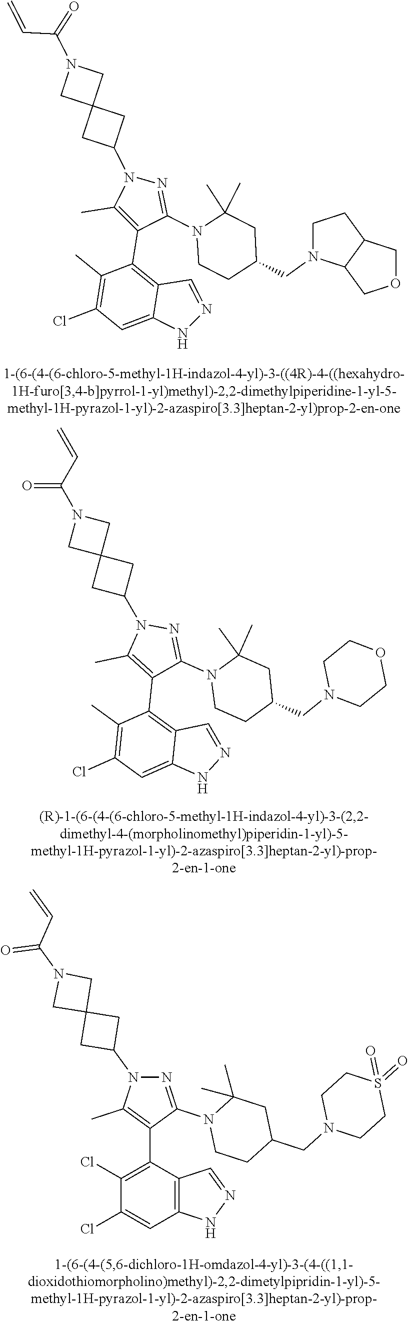 [SMILES: C=CC(=O)N1CC2(CC(n3nc(N4CC[C@@H](CN5CCC6COCC65)CC4(C)C)c(-c4c(C)c(Cl)cc5[nH]ncc45)c3C)C2)C1 ; C=CC(=O)N1CC2(CC(n3nc(N4CC[C@@H](CN5CCOCC5)CC4(C)C)c(-c4c(C)c(Cl)cc5[nH]ncc45)c3C)C2)C1 ; C=CC(=O)N1CC2(CC(n3nc(N4CCC(CN5CCS(=O)(=O)CC5)CC4(C)C)c(-c4c(Cl)c(Cl)cc5[nH]ncc45)c3C)C2)C1] 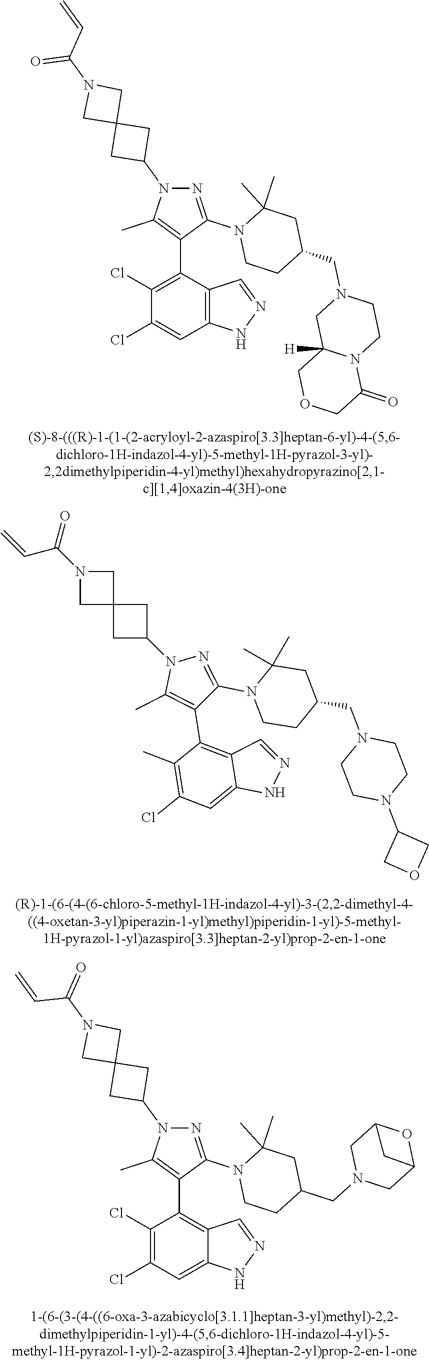 [SMILES: C=CC(=O)N1CC2(CC(n3nc(N4CC[C@@H](CN5CCN6C(=O)COC[C@@H]6C5)CC4(C)C)c(-c4c(Cl)c(Cl)cc5[nH]ncc45)c3C)C2)C1 ; C=CC(=O)N1CC2(CC(n3nc(N4CC[C@@H](CN5CCN(C6COC6)CC5)CC4(C)C)c(-c4c(C)c(Cl)cc5[nH]ncc45)c3C)C2)C1 ; C=CC(=O)N1CC2(CC(n3nc(N4CCC(CN5CC6CC(C5)O6)CC4(C)C)c(-c4c(Cl)c(Cl)cc5[nH]ncc45)c3C)C2)C1] 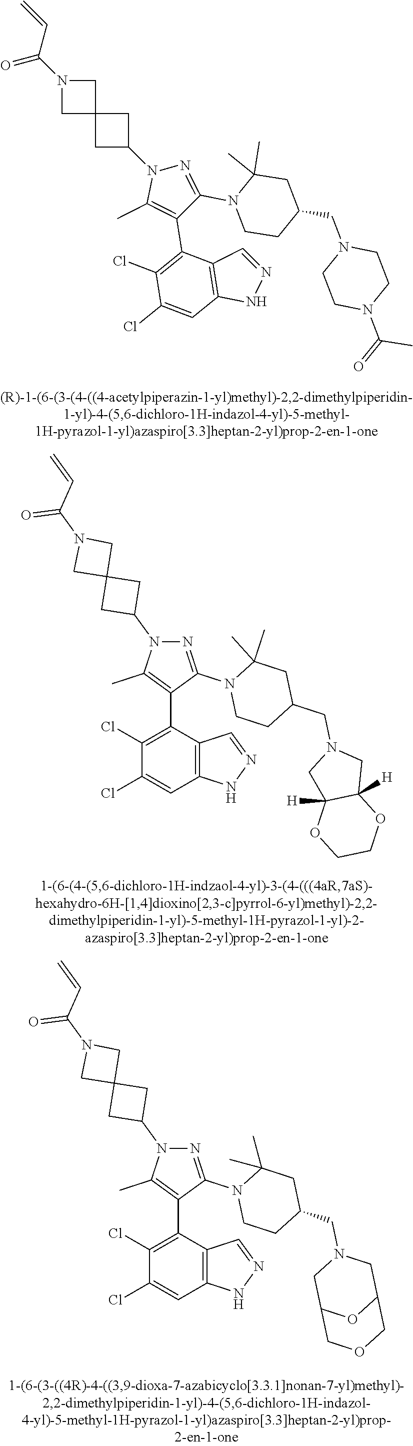 [SMILES: C=CC(=O)N1CC2(CC(n3nc(N4CC[C@@H](CN5CCN(C(C)=O)CC5)CC4(C)C)c(-c4c(Cl)c(Cl)cc5[nH]ncc45)c3C)C2)C1 ; C=CC(=O)N1CC2(CC(n3nc(N4CCC(CN5C[C@@H]6OCCO[C@@H]6C5)CC4(C)C)c(-c4c(Cl)c(Cl)cc5[nH]ncc45)c3C)C2)C1 ; C=CC(=O)N1CC2(CC(n3nc(N4CC[C@@H](CN5CC6COCC(C5)O6)CC4(C)C)c(-c4c(Cl)c(Cl)cc5[nH]ncc45)c3C)C2)C1] 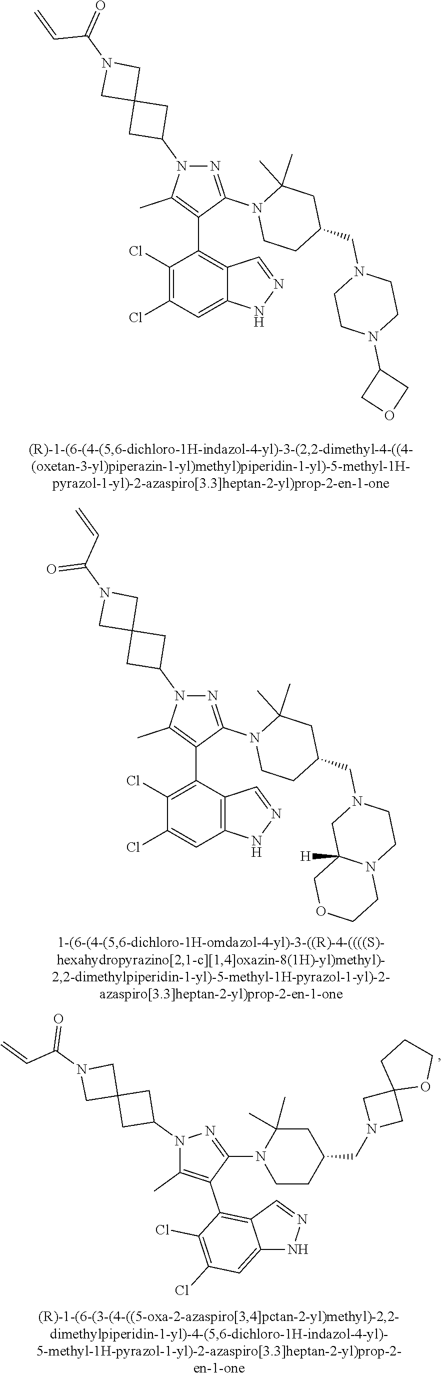 [SMILES: C=CC(=O)N1CC2(CC(n3nc(N4CC[C@@H](CN5CCN(C6COC6)CC5)CC4(C)C)c(-c4c(Cl)c(Cl)cc5[nH]ncc45)c3C)C2)C1 ; C=CC(=O)N1CC2(CC(n3nc(N4CC[C@@H](CN5CCN6CCOC[C@@H]6C5)CC4(C)C)c(-c4c(Cl)c(Cl)cc5[nH]ncc45)c3C)C2)C1 ; C=CC(=O)N1CC2(CC(n3nc(N4CC[C@@H](CN5CC6(CCCO6)C5)CC4(C)C)c(-c4c(Cl)c(Cl)cc5[nH]ncc45)c3C)C2)C1]
or a pharmaceutically acceptable salt thereof.

58. A pharmaceutical composition comprising a compound according to claim 1, or a pharmaceutically acceptable salt thereof and at least one pharmaceutically acceptable carrier.

59-60. (canceled)

61. A method of treating cancer, the method comprising administering a therapeutically effective amount of a compound or a pharmaceutically acceptable salt thereof according to claim 1 to a patient in need thereof.

62-63. (canceled)

64. The method of claim 61, wherein the cancer is selected from the group consisting of lung cancer, colorectal cancer, pancreatic cancer, uterine cancer and rectal cancer.

65. The method of claim 64, wherein the cancer is mediated by a KRAS, NRAS or GRAS G12C mutation.

66. A combination comprising a compound or pharmaceutically acceptable salt thereof according to claim 1 and one or more therapeutically active agents.

67. (canceled)


SEQUENCE LISTING (1 sequence)

SEQ ID NO: 1 - DNA, 5128 bp, Artificial Sequence
  Synthetic polynucleotide
atgcgtccggcgtagaggatcgagatcgatctcgatcccgcgaaattaatacgactcactataggggaattgtgagcggataacaattcccctctagaaataattttgtttaactttaagaaggagatatacatatgaaaacacatcatcatcatcatcatggtggcggcagtggcggtggctcaggcggtggttctctcgaggttctgttccagggtccgggtttgaacgacatcttcgaagctcagaagatcgaatggcacgagggtggcggtagtggtggtggctctatgactgaatacaagctggttgttgttggtgcttgtggcgttggtaagagcgcactgaccatccagctcattcagaatcacttcgtggacgagtacgacccgaccatcgaagattcttaccgtaaacaggtggttattgatggcgaaacctgtctgctggatattctggacactgctggtcaggaagagtactccgctatgcgtgatcagtacatgcgtactggtgaaggtttcctctgcgtgttcgctatcaacaacaccaagtccttcgaagatatccaccattaccgtgaacagatcaaacgtgtgaaggacagcgaagacgtgccaatggttctggtgggcaacaaatgtgatctcccgagccgtaccgttgacaccaaacaggcacaagacctggcacgttcctacggcatcccattcattgaaactagcgcgaagactcgtcagggtgtggacgacgcattctacactctggtgcgtgaaattcgcaagcacaaagagaaataatggtaccgaattcgcggccgcctgcagcctaggctgctaaacaaagcccgaaaggaagctgagttggctgctgccaccgctgagcaataactagcataaccccttggggcctctaaacgggtcttgaggggttttttgctgaaaggaggaactatatccggattggcgaatgggacgcgccctgtagcggcgcattaagtgcagcgtcaaaagggcgacacaaaatttattctaaatgcataataaatactgataacatcttatagtttgtattatattttgtattatcgttgacatgtataattttgatatcaaaaactgattttccctttattattttcgagatttattttcttaattctctttaacaaactagaaatattgtatatacaaaaaatcataaataatagatgaatagtttaattataggtgttcatcaatcgaaaaagcaacgtatcttatttaaagtgcgttgcttttttctcatttataaggttaaataattctcatatatcaagcaaagtgacaggcgcccttaaatattctgacaaatgctctttccctaaactccccccataaaaaaacccgccgaagcgggtttttacgttatttgcggattaacgattactcgttatcagaaccgcccagggggcccgagcttaagactggccgtcgttttacaacacagaaagagtttgtagaaacgcaaaaaggccatccgtcaggggccttctgcttagtttgatgcctggcagttccctactctcgccttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtgggctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgacgcgcgcgtaactcacgttaagggattttggtcatgagcttgcgccgtcccgtcaagtcagcgtaatgctctgcttttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgaggcgaaatacgcgatcgctgttaaaaggacaattacaaacaggaatcgagtgcaaccggcgcaggaacactgccagcgcatcaacaatattttcacctgaatcaggatattcttctaatacctggaacgctgtttttccggggatcgcagtggtgagtaaccatgcatcatcaggagtacggataaaatgcttgatggtcggaagtggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatattcttcctttttcaatattattgaagcatttatcagggttattgtctcatgagcggatacatatttgaatgtatttagaaaaataaacaaataggggtcagtgttacaaccaattaaccaattctgaacattatcgcgagcccatttatacctgaatatggctcataacaccccttgtttgcctggcggcagtagcgcggtggtcccacctgaccccatgccgaactcagaagtgaaacgccgtagcgccgatggtagtgtggggactccccatgcgagagtagggaactgccaggcatcaaataaaacgaaaggctcagtcgaaagactgggcctttcgcccgggctaattagggggtgtcgcccttcgctgaagaattgatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgacacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacg